(19) United States
(12) Patent Application Publication
(10) Pub. No.: US 2025/0368647 A1
(43) Pub. Date: Dec. 4, 2025

(54) NITROGEN CONTAINING CONDENSED 2,3-DIHYDROQUINAZOLINONE COMPOUNDS AS NAV1.8 INHIBITORS

(71) Applicant: GLAXOSMITHKLINE INTELLECTUAL PROPERTY DEVELOPMENT LIMITED, Stevenage (GB)

(72) Inventors: Jie GUANG, Collegeville, PA (US); Ming-Hsun HO, Collegeville, PA (US); Jay M. MATTHEWS, Collegeville, PA (US); Alan T. PRICE, Collegeville, PA (US); Robert SANCHEZ, Collegeville, PA (US); Jared Troy SPLETSTOSER, Collegeville, PA (US); David Glenn WASHBURN, Collegeville, PA (US); Stephen Marion BIERSCHENK, Collegeville, PA (US); Roderick S. DAVIS, Collegeville, PA (US); Marcus FARMER, Collegeville, PA (US); Mei LI, Collegeville, PA (US); Xiangmin LIAO, Collegeville, PA (US); Joseph J. ROMANO, Collegeville, PA (US); Mark J. SCHULZ, Collegeville, PA (US)

(21) Appl. No.: 18/872,308

(22) PCT Filed: Jun. 7, 2023
(86) PCT No.: PCT/IB2023/055889

§ 371 Date: Dec. 6, 2024

Related U.S. Application Data
(60) Provisional application No. 63/350,624, filed on Jun. 9, 2022.

(51) Int. Cl. C07D 471/16; A61K 31/519; C07D 498/16

(52) U.S. Cl. CPC C07D 471/16; A61K 31/519; C07D 498/16

(57) ABSTRACT

Small molecule inhibitors of Naᵥ1.8 voltage-gated sodium ion channel, including compounds of formula (I), (II), (III), (IV), and (V) are described. Also described are pharmaceutical compositions containing a compound of formula (I), (II), (III), (IV), and (V) and uses of the compounds and pharmaceutical compositions for inhibiting Naᵥ1.8 voltage-gated sodium channels and treating Naᵥ1.8 mediated diseases, such as pain and pain-associated diseases and cardiovascular diseases, such as atrial fibrillation.

### FIELD OF THE INVENTION

The invention relates to Naᵥ1.8 inhibitor compounds or pharmaceutically acceptable salts or tautomer forms thereof, corresponding pharmaceutical compositions or formulations, methods or processes of compound preparation, methods, compounds for use in, uses for and/or combination therapies for treating pain and pain-associated diseases and cardiovascular diseases.

### BACKGROUND OF THE INVENTION

Pain is a protective mechanism by which animals avoid potential tissue damage, however there are numerous disease indications in which pain outlives its usefulness and becomes a disabling burden. Indications in which pain outlives its usefulness can be broadly categorized as those in which nerve damage or injury is the trigger (neuropathic pain), those in which an inflammatory response or metabolic dysregulation sensitizes the pain response (inflammatory pain) and those in which an injury or surgical procedure results in a short-term elevation of pain response (post-operative/ambulatory pain).

Voltage-gated sodium channels underlie electrical signaling in all excitable tissues by setting the threshold and underlying the upstroke of action potentials. There are nine distinct isoforms of voltage-gated sodium channels. Those designated Naᵥ1.1, Naᵥ1.7, Naᵥ1.8 and Naᵥ1.9 are principally expressed on peripheral nerves where they control neuronal excitability. Naᵥ1.5 is the main sodium channel isoform expressed in cardiac myocytes, Naᵥ1.4 is expressed and functions in skeletal muscle, whereas Naᵥ1. 1, Naᵥ1.2, Naᵥ1.3 and Naᵥ1.6 are widely expressed in the central nervous system (CNS) and to an extent in the peripheral nervous system. The principal role of these nine voltage-gated sodium channels is comparable in that they control sodium influx into cells, but their biophysical properties varies which greatly influences the physiological profile of their respective cell type (Catterall, 2012).

Currently, non-selective sodium channel inhibitors are utilized clinically as anti-arrhythmic and anti-seizure therapies, these include lidocaine, carbamazepine, amitriptyline and mexiletine. However, as these agents exhibit a lack of selectivity between the different sodium channel isoforms, their therapeutic utility is greatly reduced due to adverse side effects, largely mediated by activity in the CNS and heart. This has stimulated efforts to develop novel medicines which are selective for specific sodium channel isoforms in order to avoid side effects in the CNS and cardiovascular system.

The Naᵥ1.8 channel is expressed in neurons of the dorsal root ganglia (DRG) and highly expressed in the small diameter neurons of this tissue which form pain sensing C- and Aδ-nerve fibers (Abrahamsen, 2008; Amaya, 2000; Novakovic, 1998). The channel was proposed as a therapeutic target for analgesia as soon as it was originally cloned from rat DRG (Akopian, 1996) due to its prominent physiological role in this tissue type and restricted expression profile. Naᵥ1.8 was subsequently identified, cloned and characterized from human DRG tissue (Rabart 1998). The closest molecular relative of Naᵥ1.8 is Naᵥ1.5 which shares a sequence homology of ˜60%. Naᵥ1.8 was previously known as SNS (sensory neuron sodium channel), PN3 (peripheral nerve sodium channel type 3), and as it exhibits characteristic pharmacological properties in its resistance to block by tetrodotoxin, it is also described as a TTX-resistant sodium channel.

Support for Naᵥ1.8 as a therapeutic target for pain indications comes from several sources. Naᵥ1.8 has been shown to conduct the majority of current during upstroke of the action potential in DRG neurons (Blair & Bean, 2002) and due to its rate of re-priming is also critical for the ability of these neurons to fire repetitively (Blair and Bean, 2003). Increased expression and function of Naᵥ1.8 has been reported in response to painful stimuli such as inflammatory mediators (England 1996 & Gold 1996), nerve damage (Roza 2003 & Ruangsri 2011), and within painful neuromas (Black 2008 & Coward 2000). Knockout of the gene encoding Nav1.8 in mice resulted in a reduced pain phenotype in particular to inflammatory challenges (Akopian 1999). Knockdown of the mRNA encoding Naᵥ1.8 also resulted in reduced painful phenotypes in rodent models, particularly in neuropathic models (Lai 2002). Pharmacological intervention via selective small molecule inhibitors has demonstrated efficacy in rodent models of inflammatory pain as well as neuropathic pain (Jarvis 2007 & Payne 2015). Supporting genetic evidence for Naᵥ1.8 is also present in patients with chronic neuropathic pain where multiple gain of function mutations has been reported to be causative in episodic painful neuropathies and small fiber neuropathies (Faber 2012, Han 2014 & Eijkenboom 2018).

### SUMMARY OF THE INVENTION

Accordingly, there is a need for the development of novel compounds, particularly Naᵥ1.8 inhibitor compounds for use in the treatment of pain and pain associated diseases, and cardiovascular diseases. The invention satisfies this need by providing compounds with Naᵥ1.8 inhibitory activity and uses of such compounds in the treatment of pain and pain associated diseases, and cardiovascular diseases.

In one aspect, provided is a compound of formula (I-a):

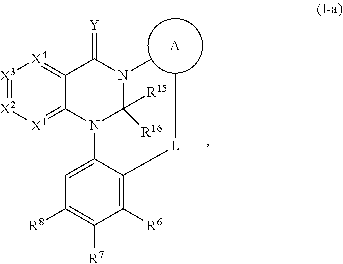

or a tautomer thereof, or a pharmaceutically acceptable salt thereof,
wherein:
   Y is O or S;
   X¹ is nitrogen or CR¹,
   X² is nitrogen or CR²,
   X³ is nitrogen or CR³, and
   X⁴ is nitrogen or CR⁴,
   provided no more than two of X¹, X², X³, and X⁴ are nitrogen;
   ring A is:

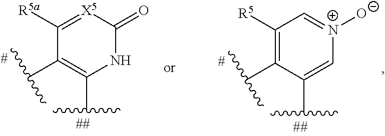

wherein [symbol] represents a covalent bond to the nitrogen atom of the bicyclic ring core of formula I and [symbol] represents a covalent bond to L of formula (I); [symbol]
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-;
each of R¹⁵ and R¹⁶ is independently hydrogen or deuterium; and
L is (C₃-C₆)alkenylene, —NHCH₂CH₂NHCH₂—, —NHCH₂CH₂OCH₂—, a divalent linker of formula (L-ia), or a divalent linker of formula (L-iia):

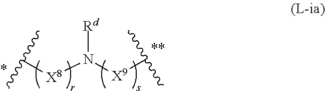

wherein:
   each X⁸ and X⁹ is independently —CR⁹R¹⁰—, wherein each of R⁹ and R¹⁰ is independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to ring A of formula (I-a) and [symbol] represents a covalent bond to the phenyl ring of formula (I-a);

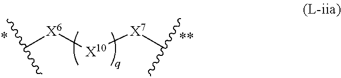

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴—, wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to ring A of formula (I-a) and [symbol] represents a covalent bond to the phenyl ring of formula (I-a).

In another aspect, provided is a pharmaceutical composition comprising a compound, or tautomer thereof, or pharmaceutically acceptable salt thereof of the invention, and a pharmaceutically acceptable excipient.

In another aspect, provided is a method of treatment of pain or a pain-associated disease in a human in need thereof, the method comprising administering to the human a compound, or tautomer thereof, or pharmaceutically acceptable salt thereof of the invention, or a pharmaceutical composition of the invention.

In another aspect, provided is a method of treatment of atrial fibrillation in a human in need thereof, the method comprising administering to the human a compound, or tautomer thereof, or pharmaceutically acceptable salt thereof of the invention, or a pharmaceutical composition of the invention.

In another aspect, provided is a compound, or tautomer thereof, or pharmaceutically acceptable salt thereof of the invention, or a pharmaceutical composition of the invention for use in therapy.

In another aspect, provided is a compound, or tautomer thereof, or pharmaceutically acceptable salt thereof of the invention, or a pharmaceutical composition of the invention for use in treatment of pain or a pain-associated disease.

In another aspect, provided is a compound, or tautomer thereof, or pharmaceutically acceptable salt thereof of the invention, or a pharmaceutical composition of the invention for use in treatment of atrial fibrillation.

In another aspect, provided is use of a compound, or tautomer thereof, or pharmaceutically acceptable salt thereof of the invention, or a pharmaceutical composition of the invention in the manufacture of a medicament for treatment of pain or a pain-associated disease.

In another aspect, provided is use of a compound, or tautomer thereof, or pharmaceutically acceptable salt thereof of the invention, or a pharmaceutical composition of the invention in the manufacture of a medicament for treatment of atrial fibrillation.

### DETAILED DESCRIPTION OF THE INVENTION

Various publications, articles and patents are cited or described in the background and throughout the specification. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification.

As used herein and in the appended claims, the singular forms “a,” “an,” and “the” include plural reference unless the context clearly dictates otherwise.

The definitions for the various groups and substituent groups of any of the Formulas disclosed herein, or a tautomer or a pharmaceutically acceptable salt thereof provided throughout the specification are intended to particularly describe each compound species disclosed herein, individually, as well as groups of one or more compound species.

The term “alkyl” refers to a saturated hydrocarbon radical, straight or branched, having the specified number of carbon atoms. For example, the term “(C₁-C₆)alkyl” refers to an alkyl group having 1 to 6 carbon atoms and the term “(C₁-C₃)alkyl” refers to an alkyl group having 1 to 3 carbon atoms. Exemplary alkyls include, but are not limited to, methyl, ethyl, n-propyl, isopropyl, n-butyl, isobutyl, s-butyl, t-butyl, pentyl, and hexyl. In some embodiments, “Me” refers to a methyl group.

When the term “alkyl” is used in combination with other substituent groups, such as “halo(C₁-C₆)alkyl” and “hydroxy(C₁-C₆)alkyl”, the term “alkyl” is intended to encompass a divalent straight or branched chain hydrocarbon radical, wherein the point of attachment is through the alkyl moiety.

The term “halo(C₁-C₆)alkyl” refers to a radical having one or more halogen atoms, which may be the same or different, at one or more carbon atoms of an alkyl moiety having 1 to 6 carbon atoms, which is a straight or branched chain carbon radical. Examples of “halo(C₁-C₆)alkyl” groups include, but are not limited to, —CH₂F (fluoromethyl), —CHF₂ (difluoromethyl), —CF₃ (trifluoromethyl), —CCl3 (trichloromethyl), 1,1-difluoroethyl, 2-fluoro-2-methylpropyl, 2,2-difluoropropyl, 2,2,2-trifluoroethyl, and hexafluoroisopropyl.

The term “alkenyl” refers to a straight or branched hydrocarbon radical containing the specified number of carbon atoms and at least 1 double bond. For example, “(C₂-C₆)alkenyl” has 2 to 6 carbon atoms. Exemplary groups include, but are not limited to, ethenyl and propenyl.

The term “alkylene” refers to a divalent radical derived from a straight chain, saturated hydrocarbon group having the specified number of carbon atoms. For example, the term “(C₃-C₆)alkylene” refers to an alkylene group having 3 to 6 carbon atoms and the term “(C₄-C₅)alkylene” refers to an alkylene group having 4 to 5 carbon atoms. Exemplary alkylene groups include, but are not limited to —CH₂CH₂CH₂—, —CH₂CH₂CH₂CH₂—, —CH₂CH₂CH₂CH₂CH₂—, and —CH₂CH₂CH₂CH₂CH₂CH₂—.

The term “alkenylene” refers to a divalent radical derived from a straight chain, unsaturated hydrocarbon group containing at least one carbon-carbon double bond and having the specified number of carbon atoms. A carbon-carbon double bond of an alkylene group can be in the cis configuration or the trans configuration, or a mixture thereof. In certain instances throughout this disclosure, an alkenylene group present as a mixture of the cis configuration and the trans configuration may be represented as

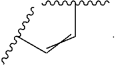 [SMILES: *C=C*]

For example, the term “(C₃-C₆)alkenylene” refers to an alkenylene group having 3 to 6 carbon atoms and at least one carbon-carbon double bond. The term “(C₄-C₅)alkenylene” refers to an alkenylene group having 4 to 5 carbon atoms and at least one carbon-carbon double bond. Exemplary alkenylene groups include, but are not limited to: —CH═CH—CH₂—, —CH₂—CH═CH—CH₂—, —CH₂CH₂—CH═CH—CH₂—, and —CH₂—CH═CH—CH₂CH₂CH₂—.

The term “alkoxy” refers to an —O-alkyl group, i.e., an alkyl group which is attached through an oxygen linking atom, wherein “alkyl” is defined above. For example, the term “(C₁-C₆)alkoxy” refers to a straight or branched chain carbon radical having 1 to 6 carbon atoms attached through an oxygen linking atom. Exemplary “(C₁-C₆)alkoxy” groups include, but are not limited to, methoxy, ethoxy, n-propoxy, isopropoxy, n-butoxy, s-butoxy, isobutoxy, and t-butoxy.

The term “halo(C₁-C₆)alkoxy” refers to a straight or branched chain hydrocarbon radical, having at least 1 and up to 6 carbon atoms with one or more halogen atoms, which may be the same or different, attached to one or more carbon atoms, which radical is attached through an oxygen linking atom. Exemplary groups include, but are not limited to, —OCHF₂ (difluoromethoxy), —OCF₃ (trifluoromethoxy), and OCH(CF₃)₂ (hexafluoroisopropoxy).

The terms “halogen” and “halo” represent chloro (—C₁), fluoro (—F), bromo (—Br), or iodo (—I) substituents.

The term “cyano” refers to the group —CN.

The term “independently selected” means that where more than one substituent is selected from a number of possible substituents, those substituents may be the same or different.

Thus, each substituent is separately selected from the entire group of recited possible substituents.

As used herein, the term “optionally” means that the subsequently described event(s) may or may not occur, and includes both event(s) that occur and event(s) that do not occur.

The term “optionally substituted” indicates that a group may be unsubstituted or substituted with one or more of the defined substituents. The term “substituted” in reference to a group indicates that a hydrogen atom attached to a member atom within a group is replaced by one of the defined substituents. In the case where groups may be selected from a number of alternative groups, the selected groups may be the same or different.

### Compounds

In one aspect, the invention relates to a compound of formula (I-a):

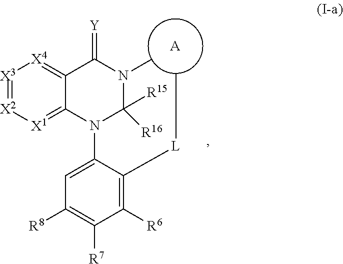

or a tautomer thereof, or a pharmaceutically acceptable salt thereof,
wherein:
   Y is O or S;
   X¹ is nitrogen or CR¹,
   X² is nitrogen or CR²,
   X³ is nitrogen or CR³, and
   X⁴ is nitrogen or CR⁴,
   provided no more than two of X¹, X², X³, and X⁴ are nitrogen;
   ring A is:

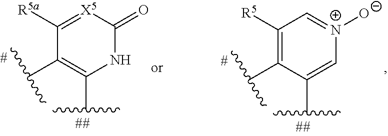

wherein [symbol] represents a covalent bond to the nitrogen atom of the bicyclic ring core of formula (I-a) and [symbol] represents a covalent bond to L of formula (I-a);
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-;
each of R¹⁵ and R¹⁶ is independently hydrogen or deuterium;
and
   L is (C₃-C₆)alkenylene, —NHCH₂CH₂NHCH₂—, —NHCH₂CH₂OCH₂—, a divalent linker of formula (L-ia), or a divalent linker of formula (L-iia):

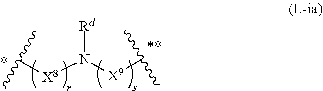

wherein:
   each X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to ring A of formula (I-a) and [symbol] represents a covalent bond to the phenyl ring of formula (I-a);

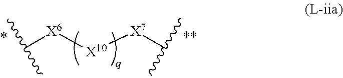

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to ring A of formula (I-a) and [symbol] represents a covalent bond to the phenyl ring of formula (I-a).

In another aspect, the invention relates to a compound of Formula (I):

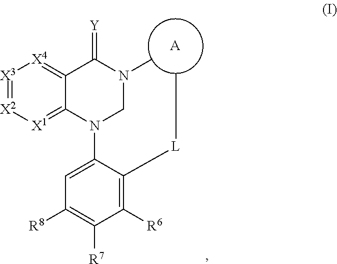

or a tautomer thereof, or a pharmaceutically acceptable salt thereof,
wherein:
   Y is O or S;
   X¹ is nitrogen or CR¹,
   X² is nitrogen or CR²,
   X³ is nitrogen or CR³, and
   X⁴ is nitrogen or CR⁴,
   provided no more than two of X¹, X², X³, and X⁴ are nitrogen;
   ring A is:

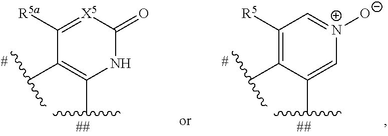

wherein [symbol] represents a covalent bond to the nitrogen atom of the bicyclic ring core of formula (I) and [symbol] represents a covalent bond to L of formula (I);
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-;
and
   L is (C₃-C₆)alkenylene, —NHCH₂CH₂NHCH₂—, —NHCH₂CH₂OCH₂—, a divalent linker of formula (L-ia), or a divalent linker of formula (L-iia):

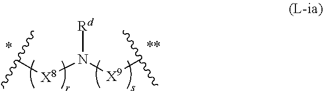

wherein:
   each X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to ring A of formula (I) and [symbol] represents a covalent bond to the phenyl ring of formula (I);

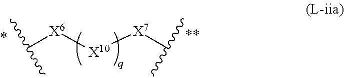

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to ring A of formula (I) and [symbol] represents a covalent bond to the phenyl ring of formula (I).

In another aspect, the invention relates to a compound of Formula (I):

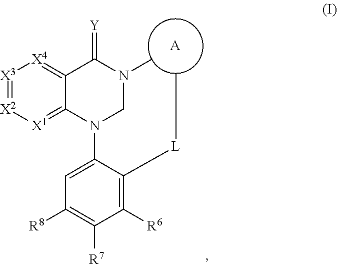

or a tautomer thereof, or a pharmaceutically acceptable salt thereof,
wherein:
   Y is O or S;
   X¹ is nitrogen or CR¹,
   X² is nitrogen or CR²,
   X³ is nitrogen or CR³, and
   X⁴ is nitrogen or CR⁴,
   provided no more than two of X¹, X², X³, and X⁴ are nitrogen;
   ring A is:

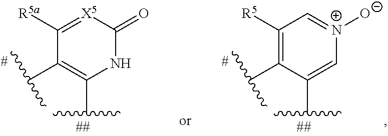

wherein [symbol] represents a covalent bond to the nitrogen atom of the bicyclic ring core of formula (I) and [symbol] represents a covalent bond to L of formula (I);
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-; and
L is (C₃-C₆)alkenylene, a divalent linker of formula (L-i), or a divalent linker of formula (L-ii):

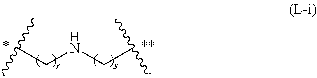

wherein:
   r is 1, 2, 3, or 4;
   s is 1, 2, 3, or 4;
   the sum of r and s is 2, 3, 4, or 5; and
   [symbol] represents a covalent bond to ring A of formula (I) and [symbol] represents a covalent bond to the phenyl ring of formula (I);

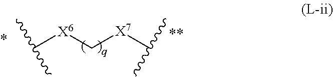

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to ring A of formula (I) and [symbol] represents a covalent bond to the phenyl ring of formula (I).

In an embodiment of a compound of formula (I-a) or formula (I), or a tautomer thereof, or a pharmaceutically acceptable salt thereof, Y is O. In another embodiment, Y is S.

In an embodiment of a compound of formula (I-a) or formula (I), or a tautomer thereof, or a pharmaceutically acceptable salt thereof, ring A is

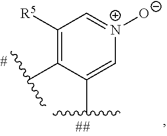

wherein R⁵ is hydrogen, halo, or —(C₁-C₆)alkyl; and [symbol] represents a covalent bond to the nitrogen atom of the bicyclic ring core of formula (I-a) or formula (I) and [symbol] represents a covalent bond to L of formula (I-a) or formula (I). In another embodiment, ring A is

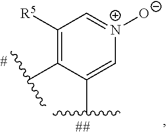

wherein R⁵ is hydrogen, —F, or —CH₃; and [symbol] represents a covalent bond to the nitrogen atom of the bicyclic ring core of formula (I-a) or formula (I) and [symbol] represents a covalent bond to L of formula (I-a) or formula (I). In another embodiment, ring A is

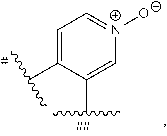

wherein [symbol] represents a covalent bond to the nitrogen atom of the bicyclic ring core of formula (I-a) or formula (I) and [symbol] represents a covalent bond to L of formula (I-a) or formula (I).

In an embodiment of a compound of formula (I-a) or formula (I), or a tautomer thereof, or a pharmaceutically acceptable salt thereof, ring A is

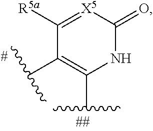

wherein X⁵ is N or CR⁵; each of R⁵ and R^{5a} is independently hydrogen, halo, or —(C₁-C₆)alkyl; and [symbol] represents a covalent bond to the nitrogen atom of the bicyclic ring core of formula (I-a) or formula (I) and [symbol] represents a covalent bond to L of formula (I-a) or formula (I). In another embodiment, ring A

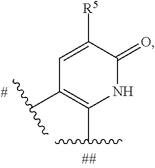

wherein R⁵ is hydrogen, halo, or —(C₁-C₆)alkyl; and [symbol] represents a covalent bond to the nitrogen atom of the bicyclic ring core of formula (I-a) or formula (I) and [symbol] represents a covalent bond to L of formula (I-a) or formula (I). In another embodiment, ring A is

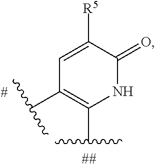

wherein R⁵ is hydrogen or —(C₁-C₆)alkyl; and [symbol] represents a covalent bond to the nitrogen atom of the bicyclic ring core of formula (I-a) or formula (I) and [symbol] represents a covalent bond to L of formula (I-a) or formula (I). In another embodiment, ring A is

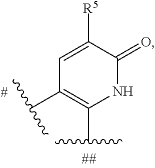

wherein R⁵ is hydrogen, —I, —Cl, or —CH₃; and [symbol] represents a covalent bond to the nitrogen atom of the bicyclic ring core of formula (I-a) or formula (I) and [symbol] represents a covalent bond to L of formula (I-a) or formula (I). In another embodiment, ring A is

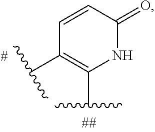

wherein [symbol] represents a covalent bond to the nitrogen atom of the bicyclic ring core of formula (I-a) or formula (I) and [symbol] represents a covalent bond to L of formula (I-a) or formula (I).

In an embodiment of a compound of formula (I-a) or formula (I), or a tautomer thereof, or a pharmaceutically acceptable salt thereof, only one of X¹, X², X³, and X⁴ is nitrogen.

In an embodiment of a compound of formula (I-a) or formula (I), or a tautomer thereof, or a pharmaceutically acceptable salt thereof, each of R¹, R², R³, and R⁴ is independently hydrogen, halo, cyano, —(C₁-C₆)alkyl, —(C₁-C₆)alkoxy, halo(C₁-C₆)alkyl, or halo(C₁-C₆)alkoxy-.

In another embodiment, each of R¹, R², R³, and R⁴ is independently hydrogen, halo, cyano, halo(C₁-C₆)alkyl, or halo(C₁-C₆)alkoxy-. In another embodiment, each of R¹, R², R³, and R⁴ is independently —F, —Cl, cyano, —CF₃, or —OCF₃. In another embodiment, each of R¹, R², R³, and R⁴ is independently hydrogen, —F, or —CF₃. In a further embodiment, R¹ and R⁴ are hydrogen, R² is —CF₃, and R³ is —F.

In an embodiment of a compound of formula (I-a) or formula (I), or a tautomer thereof, or a pharmaceutically acceptable salt thereof, each of R^{a} and R^{b} is independently hydrogen or —(C₁-C₆)alkyl. In another embodiment, each of R and R is independently hydrogen or —CH₃.

In an embodiment of a compound of formula (I-a) or formula (I), or a tautomer thereof, or a pharmaceutically acceptable salt thereof, each of R⁶, R⁷ and R⁸ is independently hydrogen, halo, halo(C₁-C₆)alkyl-, or halo(C₁-C₆)alkoxy-. In another embodiment, each of R⁶, R⁷ and R⁸ is independently hydrogen, —F, —CF₃, or —OCF₃.

In an embodiment of a compound of formula (I-a) or formula (I), or a tautomer thereof, or a pharmaceutically acceptable salt thereof, R⁸ is hydrogen and each of R⁶ and R⁷ is independently hydrogen, halo, —(C₁-C₆)alkyl, —(C₁-C₆)alkoxy, halo(C₁-C₆)alkyl-, or halo(C₁-C₆)alkoxy-. In another embodiment, R⁸ is hydrogen and each of R⁶ and R⁷ is independently hydrogen, halo, halo(C₁-C₆)alkyl-, or halo(C₁-C₆)alkoxy-. In another embodiment, R⁸ is hydrogen and each of R⁶ and R⁷ is independently hydrogen, —F, —CF₃, or —OCF₃. In a further embodiment, R⁸ is hydrogen and each of R⁶ and R⁷ is —F.

In an embodiment of a compound of formula (I-a) or formula (I), or a tautomer thereof, or a pharmaceutically acceptable salt thereof, each of R⁶ and R⁸ is hydrogen, and R⁷ is hydrogen, halo, —(C₁-C₆)alkyl, —(C₁-C₆)alkoxy, halo(C₁-C₆)alkyl-, or halo(C₁-C₆)alkoxy-. In another embodiment, each of R⁶ and R⁸ is hydrogen, and R⁷ is hydrogen, halo, halo(C₁-C₆)alkyl-, or halo(C₁-C₆)alkoxy-. In another embodiment, each of R⁶ and R⁸ is hydrogen, and R⁷ is hydrogen, —F, —CF₃, or —OCF₃. In another embodiment, each of R⁶ and R⁸ is hydrogen, and R⁷ is —F, —CF₃, or —OCF₃. In another embodiment, each of R⁶ and R⁸ is hydrogen, and R⁷ is —F.

In an embodiment of a compound of formula (I-a) or formula (I), or a tautomer thereof, or a pharmaceutically acceptable salt thereof, L is a divalent linker of 3 to 6 atoms in length, such as 3, 4, 5, or 6 atoms in length. In some embodiments, L is a divalent linker of 4 to 5 atoms in length. In some embodiments, L is a divalent linker of 4 atoms in length. In some embodiments, L is a divalent linker of 5 atoms in length.

In an embodiment of a compound of formula (I-a) or formula (I), or a tautomer thereof, or a pharmaceutically acceptable salt thereof, L is (C₃-C₆)alkenylene, such as a C₃-alkenylene, C₄-alkenylene, C₅-alkenylene or C₆-alkenylene. In another embodiment, L is (C₃-C₆)alkenylene having one carbon-carbon double bond. In another embodiment, L is (C₄-C₆)alkenylene. In another embodiment, L is (C₄-C₆)alkenylene having one carbon-carbon double bond. In another embodiment, L is (C₄-C₅)alkenylene. In another embodiment, L is (C₄-C₅)alkenylene having one carbon-carbon double bond. In some embodiments, when L is a (C₃-C₆)alkenylene having one carbon-carbon double bond, the carbon-carbon double bond is in the cis configuration, trans configuration, or a mixture thereof, such as a mixture of cis:trans of 2:1 to 1:2, e.g., 2:1, 1:1, or 1:2. In another embodiment, L is (C₃-C₆)alkenylene selected from the group consisting of *—CH═CH—CH₂—**, *—CH₂—CH═CH—CH₂—**, *—CH₂CH₂—CH═CH—CH₂—**, and *—CH₂—CH═CH—CH₂CH₂CH₂—**, wherein “*” represents a covalent bond to the ring A of formula (I) and “**” represents a covalent bond to the phenyl ring of formula (I). In another embodiment, L is (C₃-C₆)alkenylene selected from the group consisting of:

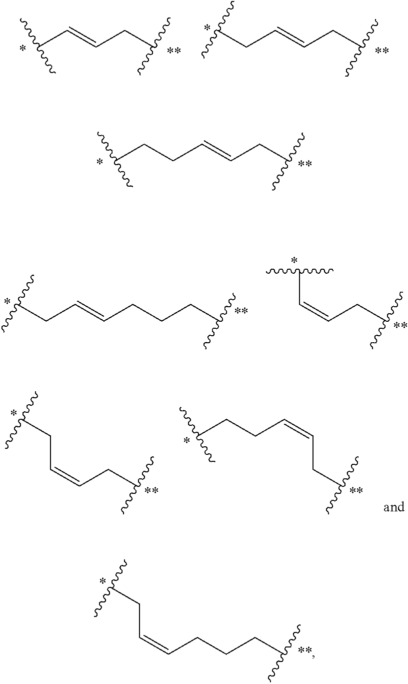

wherein [symbol] represents a covalent bond to the ring A of formula (I) and [symbol] represents a covalent bond to the phenyl ring of formula (I).

In an embodiment of a compound of formula (I-a) or formula (I), or a tautomer thereof, or a pharmaceutically acceptable salt thereof, L is a divalent linker of formula (L-ia):

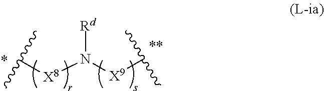

wherein:
   each X⁸ and X⁹ is independently —CR⁹R¹⁰—, wherein R⁹ and R¹⁰ are each independently hydrogen or —(C₁-C₃)alkyl;
      R^{d} is hydrogen or —(C₁-C₃)alkyl;
      r is 1, 2, 3, or 4;
      s is 1, 2, 3, or 4;
      the sum of r and s is 2, 3, 4, or 5; and
      [symbol] represents a covalent bond to ring A of formula (I-a) or formula (I) and [symbol] represents a covalent bond to the phenyl ring of formula (I-a) or formula (I).
         In another embodiment, L is a divalent linker of formula (L-ia), wherein the sum of r and s is 3 or 4. In another embodiment, L is a divalent linker of formula (L-ia), wherein R⁹ and R¹⁰ are each independently hydrogen, —CH₃, or —CH₂CH₃; and R^{d} is hydrogen or —CH₃. In another embodiment, L is a divalent linker of formula (L-ia) selected from the group consisting of:

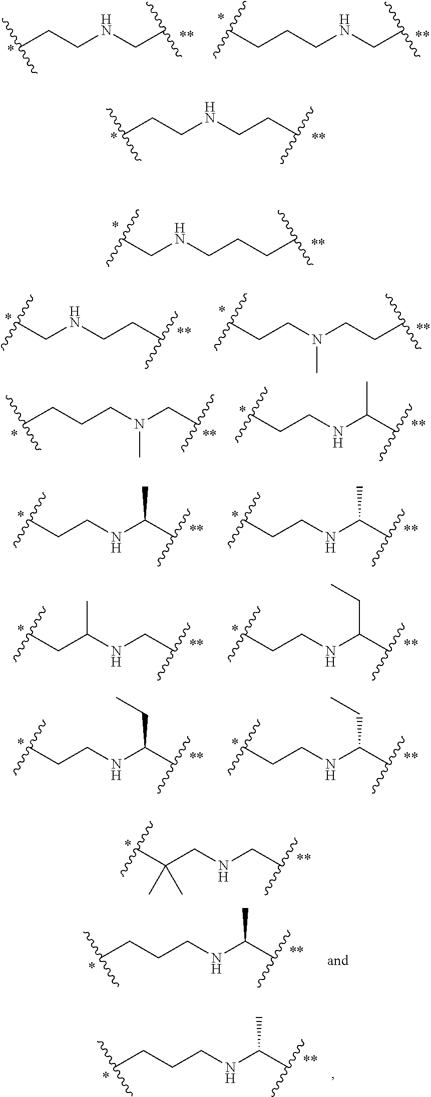

wherein [symbol] represents a covalent bond to ring A of formula (I-a) or formula (I) and [symbol] represents a covalent bond to the phenyl ring of formula (I-a) or formula (I).

In an embodiment of a compound of formula (I-a) or formula (I), or a tautomer thereof, or a pharmaceutically acceptable salt thereof, L is a divalent linker of formula (L-i):

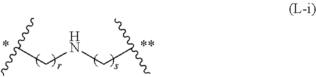

wherein:
   r is 1, 2, 3, or 4;
   s is 1, 2, 3, or 4;
   the sum of r and s is 2, 3, 4 or 5; and
   [symbol] represents a covalent bond to ring A of formula (I-a) or formula (I) and [symbol] represents a covalent bond to the phenyl ring of formula (I-a) or formula (I).
      In another embodiment, L is a divalent linker of formula (L-i), wherein the sum of r and s is 3 or 4. In another embodiment, L is a divalent linker of formula (L-i) selected from the group consisting of:

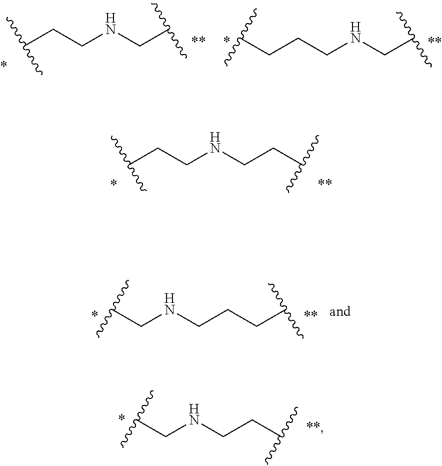

wherein [symbol] represents a covalent bond to ring A of formula (I-a) or formula (I) and [symbol] represents a covalent bond to the phenyl ring of formula (I-a) or formula (I).

In an embodiment of a compound of formula (I-a) or formula (I), or a tautomer thereof, or a pharmaceutically acceptable salt thereof, L is a divalent linker of formula (L-iia):

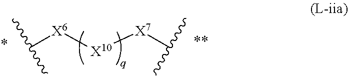

wherein:
   X⁶ is —NR— or —CH₂—;
   X⁷ is —CR¹¹R¹²—, —O— or —NH₂—, wherein R¹¹ and R¹² are each independently hydrogen or —(C₁-C₃)alkyl, provided that when X⁷ is —NH₂—, X⁶ is —CH₂—;
   each X¹⁰ is independently —CR¹³R¹⁴— wherein R¹³ and R¹⁴ are each independently hydrogen or —(C₁-C₃)alkyl;
   R^{c} is hydrogen or —(C₁-C₃)alkyl;
   q is 1, 2, 3, or 4; and
   [symbol] represents a covalent bond to ring A of formula (I-a) or formula (I) and [symbol] represents a covalent bond to the phenyl ring of formula (I-a) or formula (I).
      In another embodiment, L is a divalent linker of formula (L-iia), wherein X⁶ is —NR^{c} X⁷ is —CR¹¹R¹²—; R is hydrogen or —CH₃; and R¹¹ and R¹² are each independently hydrogen or —CH₃. In another embodiment, L is a divalent linker of formula (L-iia) selected from the group consisting of:

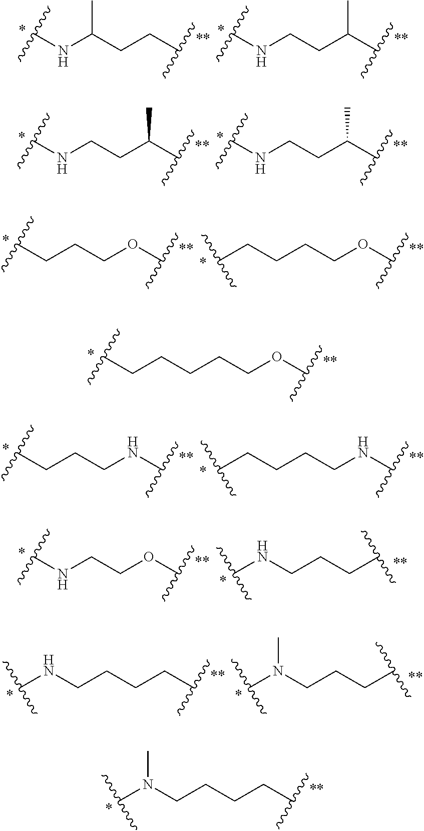

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to ring A of formula (I-a) or formula (I) and [symbol] represents a covalent bond to the phenyl ring of formula (I-a) or formula (I).

In an embodiment of a compound of formula (I-a) or formula (I), or a tautomer thereof, or a pharmaceutically acceptable salt thereof, L is a divalent linker of formula (L-ii):

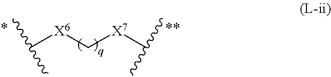

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to ring A of formula (I-a) or formula (I) and [symbol] represents a covalent bond to the phenyl ring of formula (I-a) or formula (I).
      In another embodiment, L is a divalent linker of formula (L-ii), wherein R^{c} is hydrogen or —CH₃. In another embodiment, L is a divalent linker of formula (L-ii), wherein X⁶ is —NR^{c}— and X⁷ is —CH₂—. In another embodiment, L is a divalent linker of formula (L-ii), wherein X⁶ is —NR— and X⁷ is —O—. In another embodiment, L is a divalent linker of formula (L-ii), wherein X⁶ is —CH₂— and X⁷ is —O—. In another embodiment, L is a divalent linker of formula (L-ii), wherein X⁶ is —CH₂— and X⁷ is —NH₂—. In another embodiment, L is a divalent linker of formula (L-ii), wherein X⁶ is —CH₂— and X⁷ is —CH₂—. In another embodiment, L is a divalent linker of formula (L-ii), wherein q is 2 or 3. In another embodiment, L is a divalent linker of formula (L-ii), wherein q is 2. In another embodiment, L is a divalent linker of formula (L-ii) selected from the group consisting of:

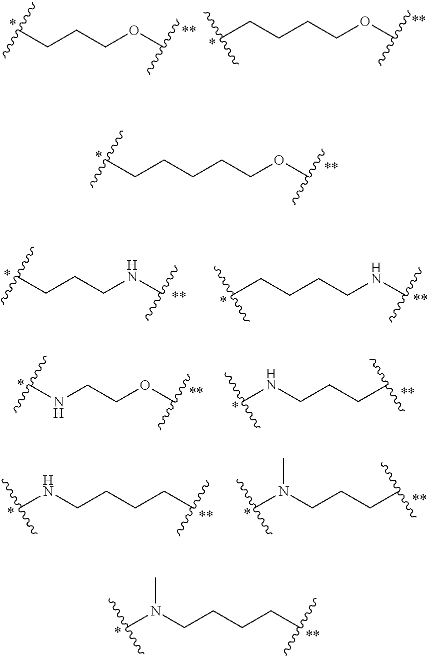

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to ring A of formula (I-a) or formula (I) and [symbol] represents a covalent bond to the phenyl ring of formula (I-a) or formula (I). In another embodiment, L is a divalent linker of formula (L-ii) selected from the group consisting of: —CH₂CH₂CH₂—, —CH₂CH₂CH₂CH₂—, —CH₂CH₂CH₂CH₂CH₂—, and —CH₂CH₂CH₂CH₂CH₂CH₂—. In another embodiment, L is a divalent linker of formula (L-ii) selected from the group consisting of:

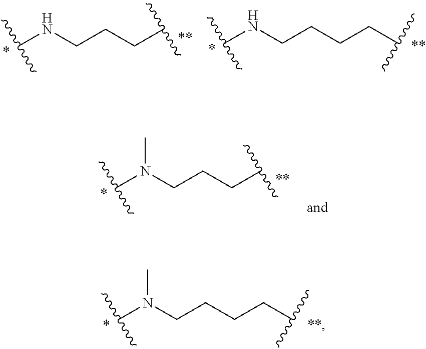

wherein [symbol] represents a covalent bond to ring A of formula (I-a) or formula (I) and [symbol] represents a covalent bond to the phenyl ring of formula (I-a) or formula (I). In another embodiment, L is a divalent linker of formula (L-ii) selected from the group consisting of;

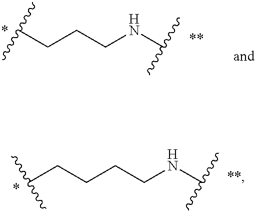

wherein [symbol] represents a covalent bond to ring A of formula (I-a) or formula (I) and [symbol] represents a covalent bond to the phenyl ring of formula (I-a) or formula (I).

In an embodiment of a compound of formula (I-a) or formula (I), or a tautomer thereof, or a pharmaceutically acceptable salt thereof, L is a divalent linker selected from the group consisting of

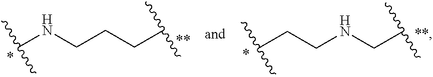

wherein [symbol] represents a covalent bond to ring A of formula (I-a) or formula (I) and [symbol] represents a covalent bond to the phenyl ring of formula (I-a) or formula (I). In another embodiment, L is the divalent linker

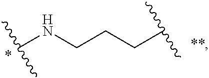

wherein [symbol] represents a covalent bond to ring A of formula (I-a) or formula (I) and [symbol] represents a covalent bond to the phenyl ring of formula (I-a) or formula (I). In another embodiment, L is the divalent linker

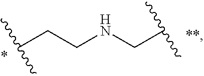

wherein [symbol] represents a covalent bond to ring A of formula (I-a) or formula (I) and [symbol] represents a covalent bond to the phenyl ring of formula (I-a) or formula (I).

In an embodiment of a compound of formula (I), or a tautomer thereof, or a pharmaceutically acceptable salt thereof:
Y is O;
X¹ is nitrogen or CR¹,
X² is nitrogen or CR²,
X³ is nitrogen or CR³, and
X⁴ is nitrogen or CR⁴,
provided no more than two of X¹, X², X³, and X⁴ are nitrogen;
ring A is

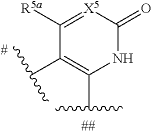

wherein [symbol] represents a covalent bond to the nitrogen atom of the bicyclic ring core of formula (I) and [symbol] represents a covalent bond to L of formula (I);
each of R¹, R², R³, and R⁴ is independently hydrogen, halo, cyano, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶ and R⁷ is independently hydrogen, halo, halo(C₁-C₆)alkyl-, or halo(C₁-C₆)alkoxy-;
R⁸ is hydrogen; and
L is a divalent linker of formula (L-i) or a divalent linker of formula (L-ii):

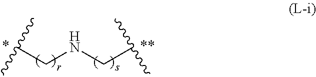

wherein:
   r is 1, 2, 3, or 4;
   s is 1, 2, 3, or 4;
   the sum of r and s is 2, 3, 4, or 5; and
   [symbol] represents a covalent bond to ring A of formula (I) and [symbol] represents a covalent bond to the phenyl ring of formula (I);

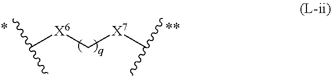

wherein:
   X⁶ is —NR—;
   X⁷ is —CH₂—;
   R^{c} is hydrogen or —(C₁-C₃)alkyl;
   q is 1, 2, 3, or 4; and
   [symbol] represents a covalent bond to ring A of formula (I) and [symbol] represents a covalent bond to the phenyl ring of formula (I).

In an embodiment of a compound of formula (I-a), or a tautomer thereof, or a pharmaceutically acceptable salt thereof, one of R¹⁵ and R¹⁶ is hydrogen and the other of R¹⁵ and R¹⁶ is deuterium. In another embodiment, each of R¹⁵ and R¹⁶ is deuterium.

The invention also relates to a compound of Formula (II):

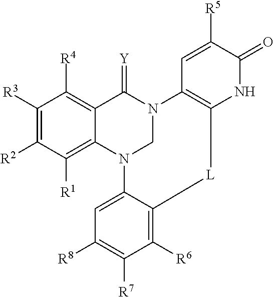

or a tautomer thereof, or a pharmaceutically acceptable salt thereof,
wherein:
   Y is O or S;
   each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, —NHCH₂CH₂NHCH₂—, —NHCH₂CH₂OCH₂—, a divalent linker of formula (L-ia), or a divalent linker of formula (L-iia):

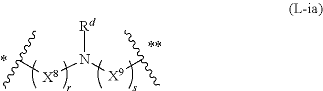

wherein:
   each of X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to the pyridone ring of formula (II) and [symbol] represents a covalent bond to the phenyl ring of formula (II);

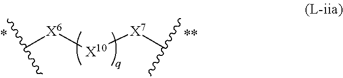

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to pyridone ring of formula (II) and [symbol] represents a covalent bond to the phenyl ring of formula (II).

The invention also relates to a compound of Formula (II):

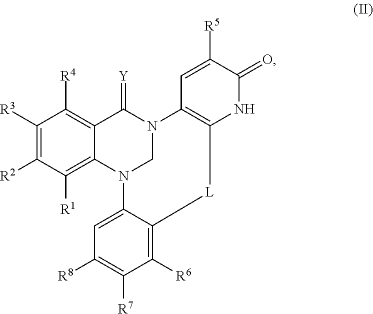

or a tautomer thereof, or a pharmaceutically acceptable salt thereof,
wherein:
   Y is O or S;
   each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, a divalent linker of formula (L-i), or a divalent linker of formula (L-ii):

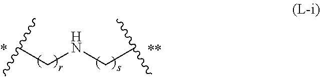

wherein:
   r is 1, 2, 3, or 4;
   s is 1, 2, 3, or 4;
   the sum of r and s is 2, 3, 4, or 5; and
   [symbol] represents a covalent bond to the pyridone ring of formula (II) and [symbol] represents a covalent bond to the phenyl ring of formula (II);

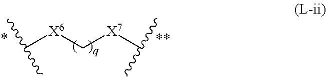

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, Y is O. In another embodiment, Y is S.

In an embodiment of a compound of formula (II), or a tautomer thereof, or a pharmaceutically acceptable salt thereof, each of R¹, R², R³, and R⁴ is independently hydrogen, halo, cyano, —(C₁-C₆)alkyl, —(C₁-C₆)alkoxy, halo(C₁-C₆)alkyl, and halo(C₁-C₆)alkoxy-. In another embodiment, each of R¹, R², R³, and R⁴ is independently hydrogen, halo, cyano, halo(C₁-C₆)alkyl, and halo(C₁-C₆)alkoxy-. In another embodiment, each of R¹, R², R³, and R⁴ is independently —F, —Cl, cyano, —CF₃, and —OCF₃. In another embodiment, each of R¹, R², R³, and R⁴ is independently hydrogen, —F, or —CF₃. In a further embodiment, R¹ and R⁴ are hydrogen, R² is —CF₃, and R³ is —F.

In an embodiment of a compound of formula (II), or a tautomer thereof, or a pharmaceutically acceptable salt thereof, R¹ is hydrogen and each of R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-. In another embodiment, R¹ is hydrogen and each of R², R³, and R⁴ is independently hydrogen, halo, cyano, halo(C₁-C₆)alkyl, or halo(C₁-C₆)alkoxy-. In another embodiment, R¹ is hydrogen and each of R², R³, and R⁴ is independently —F, —Cl, cyano, —CF₃, or —OCF_{3.}

In an embodiment of a compound of formula (II), or a tautomer thereof, or a pharmaceutically acceptable salt thereof, each of R^{a} and R^{b} is independently hydrogen or —(C₁-C₆)alkyl. In another embodiment, each of R and R is independently hydrogen or —CH₃.

In an embodiment of a compound of formula (II), or a tautomer thereof, or a pharmaceutically acceptable salt thereof, R⁵ is hydrogen or —(C₁-C₆)alkyl. In another embodiment, R⁵ is hydrogen, —I, Cl, or —CH₃. In another embodiment, R⁵ is hydrogen.

In an embodiment of a compound of formula (II), or a tautomer thereof, or a pharmaceutically acceptable salt thereof, each of R⁶, R⁷ and R⁸ is independently hydrogen, halo, halo(C₁-C₆)alkyl-, or halo(C₁-C₆)alkoxy-. In another embodiment, each of R⁶, R⁷ and R⁸ is independently hydrogen, —F, —CF₃, or —OCF₃.

In an embodiment of a compound of formula (II), or a tautomer thereof, or a pharmaceutically acceptable salt thereof, R⁸ is hydrogen and each of R⁶ and R⁷ is independently hydrogen, halo, —(C₁-C₆)alkyl, —(C₁-C₆)alkoxy, halo(C₁-C₆)alkyl-, or halo(C₁-C₆)alkoxy-. In another embodiment, R⁸ is hydrogen and each of R⁶ and R⁷ is independently hydrogen, halo, halo(C₁-C₆)alkyl-, or halo(C₁-C₆)alkoxy-. In another embodiment, R⁸ is hydrogen and each of R⁶ and R⁷ is independently hydrogen, —F, —CF₃, or —OCF₃. In a further embodiment, R⁸ is hydrogen and each of R⁶ and R⁷ is —F.

In an embodiment of a compound of formula (II), or a tautomer thereof, or a pharmaceutically acceptable salt thereof, each of R⁶ and R⁸ is hydrogen, and R⁷ is hydrogen, halo, —(C₁-C₆)alkyl, —(C₁-C₆)alkoxy, halo(C₁-C₆)alkyl-, or halo(C₁-C₆)alkoxy-. In another embodiment, each of R⁶ and R⁸ is hydrogen, and R⁷ is hydrogen, halo, halo(C₁-C₆)alkyl-, or halo(C₁-C₆)alkoxy-. In another embodiment, each of R⁶ and R⁸ is hydrogen, and R⁷ is hydrogen, —F, —CF₃, or —OCF₃. In another embodiment, each of R⁶ and R⁸ is hydrogen, and R⁷ is —F, —CF₃, or —OCF₃. In another embodiment, each of R⁶ and R⁸ is hydrogen, and R⁷ is —F.

In an embodiment of a compound of formula (II), or a tautomer thereof, or a pharmaceutically acceptable salt thereof, L is a divalent linker of 3 to 6 atoms in length, such as 3, 4, 5, or 6 atoms in length. In some embodiments, L is a divalent linker of 4 to 5 atoms in length. In some embodiments, L is a divalent linker of 4 atoms in length. In some embodiments, L is a divalent linker of 5 atoms in length.

In an embodiment of a compound of formula (II), or a tautomer thereof, or a pharmaceutically acceptable salt thereof, L is (C₃-C₆)alkenylene, such as a C₃-alkenylene, C₄-alkenylene, C₅-alkenylene or C₆-alkenylene. In another embodiment, L is (C₃-C₆)alkenylene having one carbon-carbon double bond. In another embodiment, L is (C₄-C₆)alkenylene. In another embodiment, L is (C₄-C₆)alkenylene having one carbon-carbon double bond. In another embodiment, L is (C₄-C₅)alkenylene. In another embodiment, L is (C₄-C₅)alkenylene having one carbon-carbon double bond. In some embodiments, when L is a (C₃-C₆)alkenylene having one carbon-carbon double bond, the carbon-carbon double bond is in the cis configuration, trans configuration, or a mixture thereof, such as a mixture of cis:trans of 2:1 to 1:2, e.g., 2:1, 1:1, or 1:2. In another embodiment, L is (C₃-C₆)alkenylene selected from the group consisting of *—CH═CH—CH₂—**, *—CH₂—CH═CH—CH₂—**, *—CH₂CH₂—CH═CH—CH₂—**, and *—CH₂—CH═CH—CH₂CH₂CH₂—**, wherein “*” represents a covalent bond to the pyridone ring of formula (II) and “**” represents a covalent bond to the phenyl ring of formula (II). In another embodiment, L is (C₃-C₆)alkenylene selected from the group consisting of:

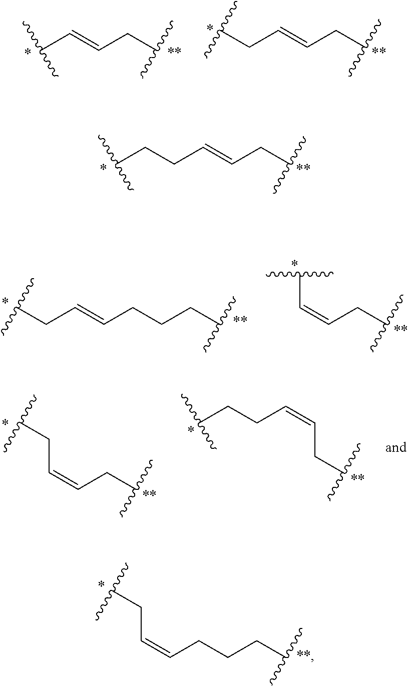

wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, L is a divalent linker of formula (L-ia):

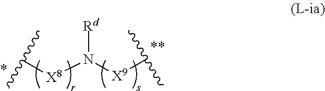

wherein:
   each of X⁸ and X⁹ is independently —CR⁹R¹⁰—, wherein each of R⁹ and R¹⁰ is independently hydrogen or —(C₁-C₃)alkyl;
   R^{d} is hydrogen or —(C₁-C₃)alkyl;
   r is 1, 2, 3, or 4;
   s is 1, 2, 3, or 4; [symbol]
   the sum of r and s is 2, 3, 4, or 5; and
   [symbol] represents a covalent bond to the pyridone ring of formula (II) and [symbol] represents a covalent bond to the phenyl ring of formula (II).
      In another embodiment, L is a divalent linker of formula (L-ia), wherein the sum of r and s is 3 or 4. In another embodiment, L is a divalent linker of formula (L-ia), wherein each of R⁹ and R¹⁰ is independently hydrogen, —CH₃, or —CH₂CH₃; and R^{d} is hydrogen or —CH₃. In another embodiment, L is a divalent linker of formula (L-ia) selected from the group consisting of:

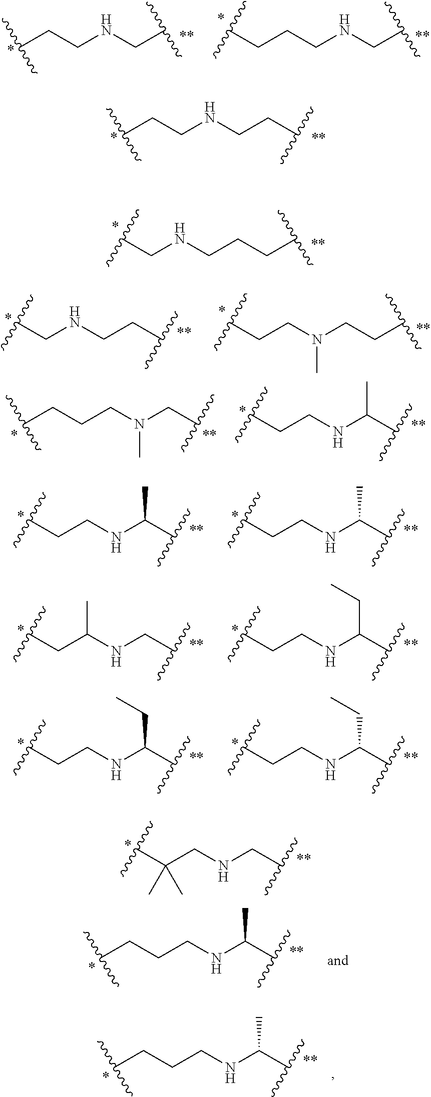

wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, L is a divalent linker of formula (L-i):

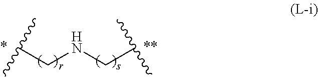

wherein:
   r is 1, 2, 3, or 4;
   s is 1, 2, 3, or 4;
   the sum of r and s is 2, 3, 4 or 5; and
   [symbol] represents a covalent bond to the pyridone ring of formula (II) and [symbol] represents a covalent bond to the phenyl ring of formula (II).
      In another embodiment, L is a divalent linker of formula (L-i), wherein the sum of r and s is 3 or 4. In another embodiment, L is a divalent linker of formula (L-i) selected from the group consisting of:

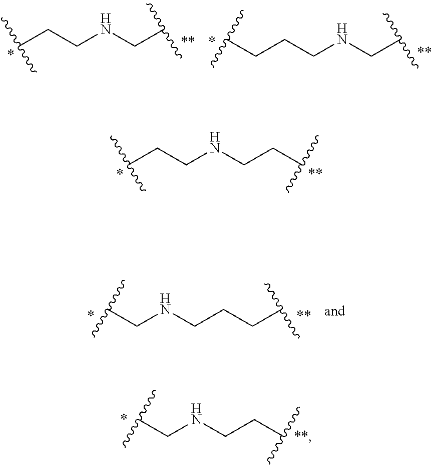

wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, L is a divalent linker of formula (L-iia):

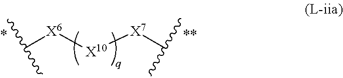

wherein:
   X⁶ is —NR— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴—, wherein each of R¹³ and R¹⁴ is independently hydrogen or —(C₁-C₃)alkyl;
   R^{c} is hydrogen or —(C₁-C₃)alkyl;
   q is 1, 2, 3, or 4; and [symbol]
   [symbol] represents a covalent bond to the pyridone ring of formula (II) and [symbol] represents a covalent bond to the phenyl ring of formula (II).
      In another embodiment, L is a divalent linker of formula (L-iia), wherein X⁶ is —NR^{c}—; X⁷ is —CR¹¹R¹²—; R^{c} is hydrogen or —CH₃; and each of R¹¹ and R¹² is independently hydrogen or —CH₃. In another embodiment, L is a divalent linker of formula (L-iia) selected from the group consisting of:

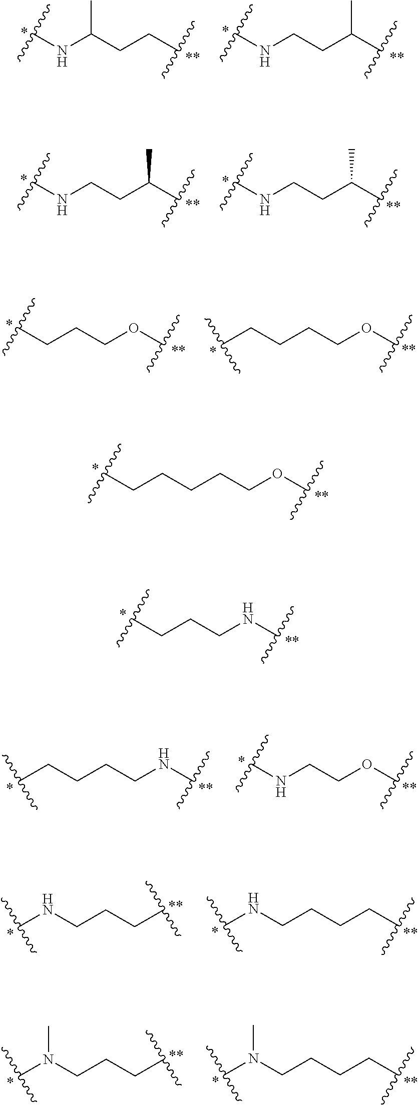

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, L is a divalent linker of formula (L-ii):

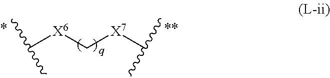

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (II) and [symbol] represents a covalent bond to the phenyl ring of formula (II).
      In another embodiment, L is a divalent linker of formula (L-ii), wherein X^{c} is hydrogen or —CH₃. In another embodiment, L is a divalent linker of formula (L-ii), wherein X⁶ is —NR^{c}— and X⁷ is —CH₂—. In another embodiment, L is a divalent linker of formula (L-ii), wherein X⁶ is —NR^{c}— and X⁷ is —O—. In another embodiment, L is a divalent linker of formula (L-ii), wherein X⁶ is —CH₂— and X⁷ is —O—. In another embodiment, L is a divalent linker of formula (L-ii), wherein X⁶ is —CH₂— and X⁷ is —NH₂—. In another embodiment, L is a divalent linker of formula (L-ii), wherein X⁶ is —CH₂— and X⁷ is —CH₂—. In another embodiment, L is a divalent linker of formula (L-ii), wherein q is 2 or 3. In another embodiment, L is a divalent linker of formula (L-ii), wherein q is 2. In another embodiment, L is a divalent linker of formula (L-ii) selected from the group consisting of:

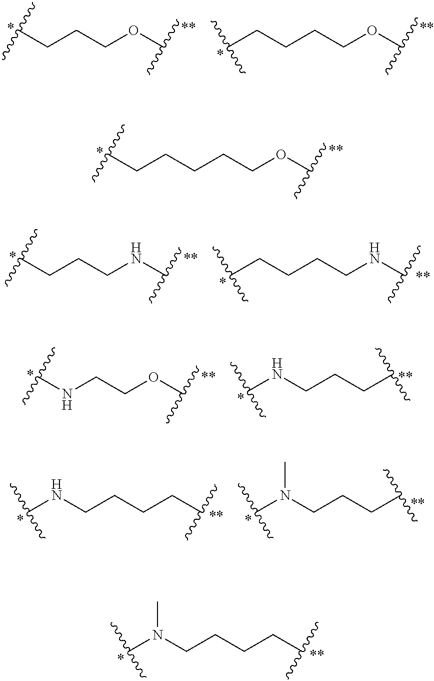

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (II) and [symbol] represents a covalent bond to the phenyl ring of formula (II). In another embodiment, L is a divalent linker of formula (L-ii) selected from the group consisting of: —CH₂CH₂CH₂—, —CH₂CH₂CH₂CH₂—, —CH₂CH₂CH₂CH₂CH₂—, and —CH₂CH₂CH₂CH₂CH₂CH₂—. In another embodiment, L is a divalent linker of formula (L-ii) selected from the group consisting of:

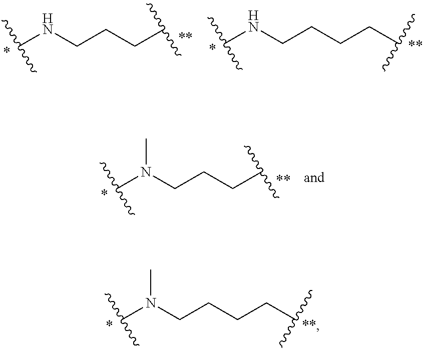

wherein [symbol] represents a covalent bond to the pyridone ring of formula (II) and [symbol] represents a covalent bond to the phenyl ring of formula (II). In another embodiment, L is a divalent linker of formula (L-ii) selected from the group consisting of:

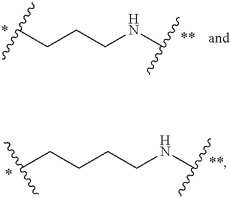

wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof, L is a divalent linker selected from the group consisting of

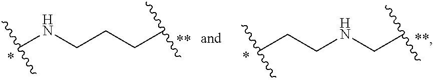

wherein [symbol] represents a covalent bond to the pyridone ring of formula (II) and [symbol] represents a covalent bond to the phenyl ring of formula (II). In another embodiment, L is the divalent linker

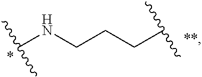

wherein [symbol] represents a covalent bond to the pyridone ring of formula (II) and [symbol] represents a covalent bond to the phenyl ring of formula (II). In another embodiment, L is the divalent linker

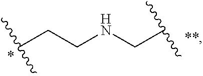

wherein [symbol] represents a covalent bond to the pyridone ring of formula (II) and [symbol] represents a covalent bond to the phenyl ring of formula (II).

In an embodiment of a compound of formula (II), or a tautomer thereof, or a pharmaceutically acceptable salt thereof:
Y is O;
R¹ is hydrogen and each of R², R³, and R⁴ is independently hydrogen, halo, cyano, halo(C₁-C₆)alkyl-, or halo(C₁-C₆)alkoxy-;
R⁵ is hydrogen;
each of R⁶ and R⁷ is independently hydrogen, halo, halo(C₁-C₆)alkyl-, or halo(C₁-C₆)alkoxy-;
R⁸ is hydrogen; and
L is a divalent linker of formula (L-i) or a divalent linker of formula (L-ii):

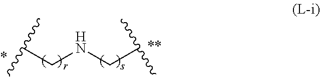

wherein:
   r is 1, 2, 3, or 4;
   s is 1, 2, 3, or 4;
   the sum of r and s is 2, 3, 4, or 5; and
   [symbol] represents a covalent bond to the pyridone ring of formula (II) and [symbol] represents a covalent bond to the phenyl ring of formula (II);

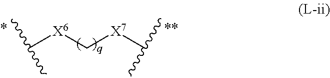

wherein:
   X⁶ is —NR—;
   X⁷ is —CH₂—;
   R^{c} is hydrogen or —(C₁-C₃)alkyl;
   q is 1, 2, 3, or 4; and
   [symbol] represents a covalent bond to the pyridone ring of formula (II) and [symbol] represents a covalent bond to the phenyl ring of formula (II).

The invention also relates to a compound of formula (III):

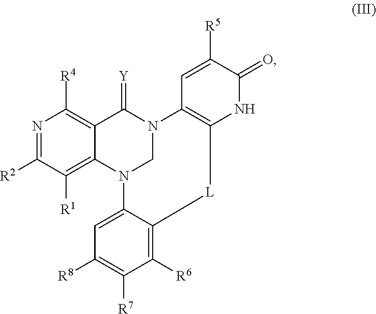

or a tautomer thereof, or a pharmaceutically acceptable salt thereof,
wherein:
   Y is O or S;
   each of R¹, R² and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, —NHCH₂CH₂NHCH₂—, —NHCH₂CH₂OCH₂—, a divalent linker of formula (L-ia), or a divalent linker of formula (L-iia):

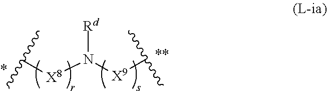

wherein:
   each of X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to the pyridone ring of formula (III) and [symbol] represents a covalent bond to the phenyl ring of formula (III);

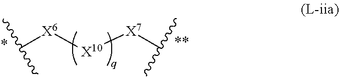

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to pyridone ring of formula (III) and [symbol] represents a covalent bond to the phenyl ring of formula (III).

The invention also relates to a compound of formula (III):

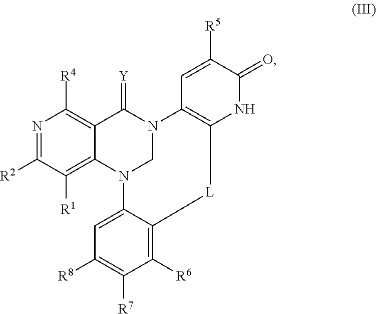

or a tautomer thereof, or a pharmaceutically acceptable salt thereof,
wherein:
   Y is O or S;
   each of R¹, R² and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, a divalent linker of formula (L-i), or a divalent linker of formula (L-ii):

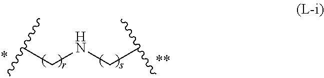

wherein:
   r is 1, 2, 3, or 4;
   s is 1, 2, 3, or 4;
   the sum of r and s is 2, 3, 4, or 5; and
   [symbol] represents a covalent bond to the pyridone ring of formula (III) and [symbol] represents a covalent bond to the phenyl ring of formula (III);

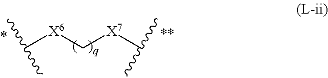

wherein:
   X⁶ is —NR^{c}— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, Y is O. In another embodiment, Y is S.

In an embodiment of a compound of formula (III), or a tautomer thereof, or a pharmaceutically acceptable salt thereof, each of R¹, R², and R⁴ is independently hydrogen, halo, cyano, —(C₁-C₆)alkyl, —(C₁-C₆)alkoxy, halo(C₁-C₆)alkyl, and halo(C₁-C₆)alkoxy-. In another embodiment, each of R¹, R², and R⁴ is independently hydrogen, halo, cyano, halo(C₁-C₆)alkyl, and halo(C₁-C₆)alkoxy-. In another embodiment, each of R¹, R², and R⁴ is independently —F, —Cl, cyano, —CF₃, and —OCF₃.

In an embodiment of a compound of formula (III), or a tautomer thereof, or a pharmaceutically acceptable salt thereof, R¹ is hydrogen and each of R² and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-. In another embodiment, R¹ is hydrogen and each of R² and R⁴ is independently hydrogen, halo, cyano, halo(C₁-C₆)alkyl, or halo(C₁-C₆)alkoxy-. In another embodiment, R¹ is hydrogen and each of R² and R⁴ is independently —F, —Cl, cyano, —CF₃, or —OCF₃.

In an embodiment of a compound of formula (III), or a tautomer thereof, or a pharmaceutically acceptable salt thereof, each of R^{a} and R^{b} is independently hydrogen or —(C₁-C₆)alkyl. In another embodiment, each of R and R is independently hydrogen or —CH₃.

In an embodiment of a compound of formula (III), or a tautomer thereof, or a pharmaceutically acceptable salt thereof, R⁵ is hydrogen or —(C₁-C₆)alkyl. In another embodiment, R⁵ is hydrogen, —I, Cl, or —CH₃. In another embodiment, R⁵ is hydrogen.

In an embodiment of a compound of formula (III), or a tautomer thereof, or a pharmaceutically acceptable salt thereof, each of R⁶, R⁷ and R⁸ is independently hydrogen, halo, halo(C₁-C₆)alkyl-, or halo(C₁-C₆)alkoxy-. In another embodiment, each of R⁶, R⁷ and R⁸ is independently hydrogen, —F, —CF₃, or —OCF₃.

In an embodiment of a compound of formula (III), or a tautomer thereof, or a pharmaceutically acceptable salt thereof, R⁸ is hydrogen and each of R⁶ and R⁷ is independently hydrogen, halo, —(C₁-C₆)alkyl, —(C₁-C₆)alkoxy, halo(C₁-C₆)alkyl-, or halo(C₁-C₆)alkoxy-. In another embodiment, R⁸ is hydrogen and each of R⁶ and R⁷ is independently hydrogen, halo, halo(C₁-C₆)alkyl-, or halo(C₁-C₆)alkoxy-. In another embodiment, R⁸ is hydrogen and each of R⁶ and R⁷ is independently hydrogen, —F, —CF₃, or —OCF₃.

In an embodiment of a compound of formula (III), or a tautomer thereof, or a pharmaceutically acceptable salt thereof, each of R⁶ and R⁸ is hydrogen, and R⁷ is hydrogen, halo, —(C₁-C₆)alkyl, —(C₁-C₆)alkoxy, halo(C₁-C₆)alkyl-, or halo(C₁-C₆)alkoxy-. In another embodiment, each of R⁶ and R⁸ is hydrogen, and R⁷ is hydrogen, halo, halo(C₁-C₆)alkyl-, or halo(C₁-C₆)alkoxy-. In another embodiment, each of R⁶ and R⁸ is hydrogen, and R⁷ is hydrogen, —F, —CF₃, or —OCF₃. In another embodiment, each of R⁶ and R⁸ is hydrogen, and R⁷ is —F, —CF₃, or —OCF₃. In another embodiment, each of R⁶ and R⁸ is hydrogen, and R⁷ is —F.

In an embodiment of a compound of formula (III), or a tautomer thereof, or a pharmaceutically acceptable salt thereof, L is a divalent linker of 3 to 6 atoms in length, such as 3, 4, 5, or 6 atoms in length. In some embodiments, L is a divalent linker of 4 to 5 atoms in length. In some embodiments, L is a divalent linker of 4 atoms in length. In some embodiments, L is a divalent linker of 5 atoms in length.

In an embodiment of a compound of formula (III), or a tautomer thereof, or a pharmaceutically acceptable salt thereof, L is (C₃-C₆)alkenylene, such as a C₃-alkenylene, C₄-alkenylene, C₅-alkenylene or C₆-alkenylene. In another embodiment, L is (C₃-C₆)alkenylene having one carbon-carbon double bond. In another embodiment, L is (C₄-C₆)alkenylene. In another embodiment, L is (C₄-C₆)alkenylene having one carbon-carbon double bond. In another embodiment, L is (C₄-C₅)alkenylene. In another embodiment, L is (C₄-C₅)alkenylene having one carbon-carbon double bond. In some embodiments, when L is a (C₃-C₆)alkenylene having one carbon-carbon double bond, the carbon-carbon double bond is in the cis configuration, trans configuration, or a mixture thereof, such as a mixture of cis:trans of 2:1 to 1:2, e.g., 2:1, 1:1, or 1:2. In another embodiment, L is (C₃-C₆)alkenylene selected from the group consisting of *—CH═CH—CH₂—**, *—CH₂—CH═CH—CH₂—**, *—CH₂CH₂—CH═CH—CH₂—**, and *—CH₂—CH═CH—CH₂CH₂CH₂—**, wherein “*” represents a covalent bond to the pyridone ring of formula (III) and “**” represents a covalent bond to the phenyl ring of formula (III). In another embodiment, L is (C₃-C₆)alkenylene selected from the group consisting of:

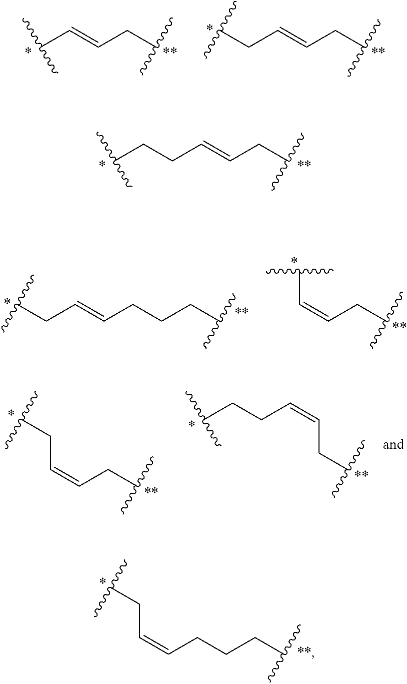

wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, L is a divalent linker of formula (L-ia):

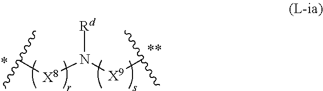

wherein:
   each of X⁸ and X⁹ is independently —CR⁹R¹⁰—, wherein each of R⁹ and R¹⁰ is independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
[symbol] represents a covalent bond to the pyridone ring of formula (III) and [symbol] represents a covalent bond to the phenyl ring of formula (III).
   In another embodiment, L is a divalent linker of formula (L-ia), wherein the sum of r and s is 3 or 4. In another embodiment, L is a divalent linker of formula (L-ia), wherein each of R⁹ and R¹⁰ is independently hydrogen, —CH₃, or —CH₂CH₃; and R^{d} is hydrogen or —CH₃. In another embodiment, L is a divalent linker of formula (L-ia) selected from the group consisting of:

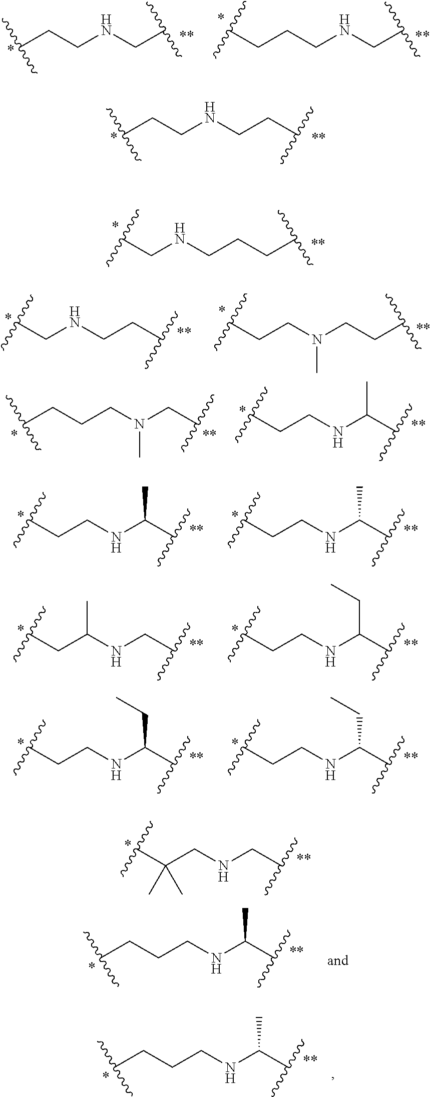

wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, L is a divalent linker of formula (L-i):

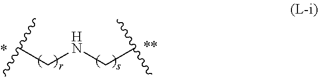

wherein:
   r is 1, 2, 3, or 4;
   s is 1, 2, 3, or 4;
   the sum of r and s is 2, 3, 4 or 5; and
   [symbol] represents a covalent bond to the pyridone ring of formula (III) and [symbol] represents a covalent bond to the phenyl ring of formula (III).
      In another embodiment, L is a divalent linker of formula (L-i), wherein the sum of r and s is 3 or 4. In another embodiment, L is a divalent linker of formula (L-i) selected from the group consisting of:

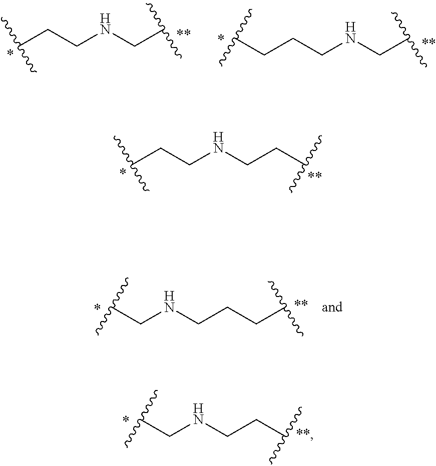

wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, L is a divalent linker of formula (L-iia):

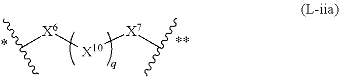

wherein:
   X⁶ is —NR— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴—, wherein each of R¹³ and R¹⁴ is independently hydrogen or —(C₁-C₃)alkyl;
   R^{c} is hydrogen or —(C₁-C₃)alkyl;
   q is 1, 2, 3, or 4; and
   [symbol] represents a covalent bond to the pyridone ring of formula (III) and [symbol] represents a covalent bond to the phenyl ring of formula (III).
      In another embodiment, L is a divalent linker of formula (L-iia), wherein X⁶ is —NR^{c}—; X⁷ is —CR¹¹R¹²—; R is hydrogen or —CH₃; and each of R¹¹ and R¹² is independently hydrogen or —CH₃.
      In another embodiment, L is a divalent linker of formula (L-iia) selected from the group consisting of:

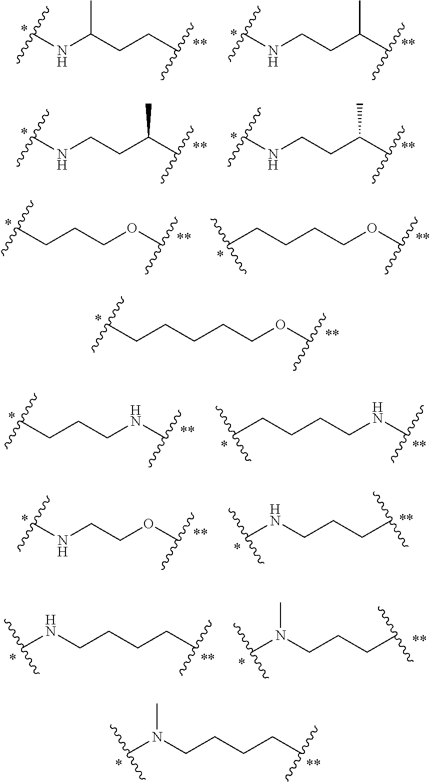

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, L is a divalent linker of formula (L-ii):

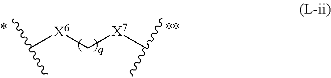

wherein:
   X⁶ is —NR^{c}— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (III) and [symbol] represents a covalent bond to the phenyl ring of formula (III).
      In another embodiment, L is a divalent linker of formula (L-ii), wherein R^{c} is hydrogen or —CH₃. In another embodiment, L is a divalent linker of formula (L-ii), wherein X⁶ is —NR^{c}— and X⁷ is —CH₂—. In another embodiment, L is a divalent linker of formula (L-ii), wherein X⁶ is —NR— and X⁷ is —O—. In another embodiment, L is a divalent linker of formula (L-ii), wherein X⁶ is —CH₂— and X⁷ is —O—. In another embodiment, L is a divalent linker of formula (L-ii), wherein X⁶ is —CH₂— and X⁷ is —NH₂—. In another embodiment, L is a divalent linker of formula (L-ii), wherein X⁶ is —CH₂— and X⁷ is —CH₂—. In another embodiment, L is a divalent linker of formula (L-ii), wherein q is 2 or 3. In another embodiment, L is a divalent linker of formula (L-ii), wherein q is 2. In another embodiment, L is a divalent linker of formula (L-ii) selected from the group consisting of:

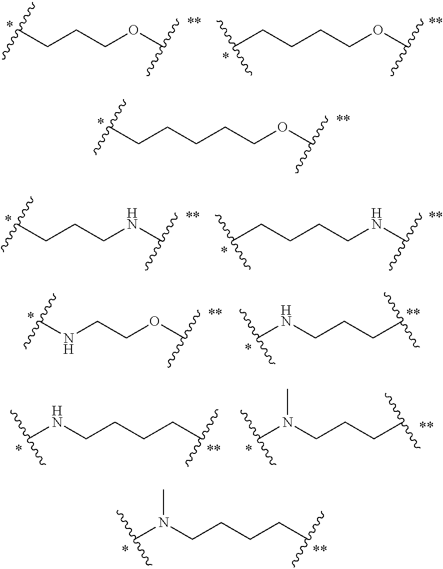

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (III) and [symbol] represents a covalent bond to the phenyl ring of formula (III). In another embodiment, L is a divalent linker of formula (L-ii) selected from the group consisting of: —CH₂CH₂CH₂—, —CH₂CH₂CH₂CH₂—, —CH₂CH₂CH₂CH₂CH₂—, and —CH₂CH₂CH₂CH₂CH₂CH₂—. In another embodiment, L is a divalent linker of formula (L-ii) selected from the group consisting of:

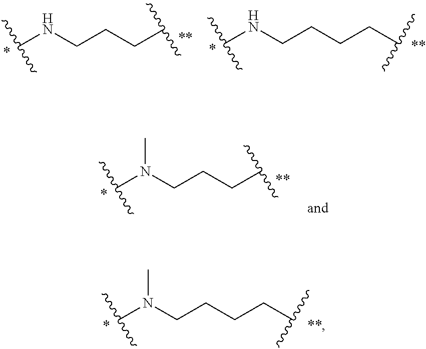

wherein [symbol] represents a covalent bond to the pyridone ring of formula (III) and [symbol] represents a covalent bond to the phenyl ring of formula (III). In another embodiment, L is a divalent linker of formula (L-ii) selected from the group consisting of:

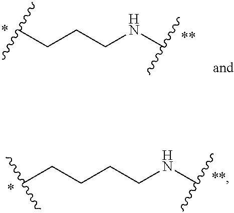

wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof, L is a divalent linker selected from the group consisting of

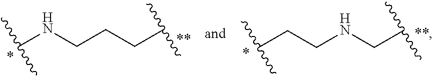

wherein [symbol] represents a covalent bond to the pyridone ring of formula (III) and [symbol] represents a covalent bond to the phenyl ring of formula (III). In another embodiment, L is the divalent linker

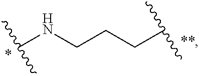

wherein [symbol] represents a covalent bond to the pyridone ring of formula (III) and [symbol] represents a covalent bond to the phenyl ring of formula (III). In another embodiment, L is the divalent linker

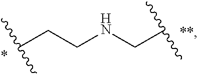

wherein [symbol] represents a covalent bond to the pyridone ring of formula (III) and [symbol] represents a covalent bond to the phenyl ring of formula (III).

In an embodiment of a compound of formula (III), or a tautomer thereof, or a pharmaceutically acceptable salt thereof:
Y is O;
R¹ is hydrogen and each of R² and R⁴ is independently hydrogen, halo, cyano, halo(C₁-C₆)alkyl-, or halo(C₁-C₆)alkoxy-;
R⁵ is hydrogen;
each of R⁶ and R⁷ is independently hydrogen, halo, halo(C₁-C₆)alkyl-, or halo(C₁-C₆)alkoxy-;
R⁸ is hydrogen; and
L is a divalent linker of formula (L-i) or a divalent linker of formula (L-ii):

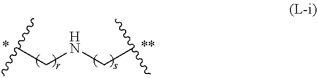

wherein:
   r is 1, 2, 3, or 4;
   s is 1, 2, 3, or 4;
   the sum of r and s is 2, 3, 4, or 5; and
   [symbol] represents a covalent bond to ring A of formula (III) and [symbol] represents a covalent bond to the phenyl ring of formula (III);

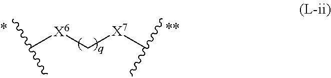

wherein:
   X⁶ is —NR—;
   X⁷ is —CH₂—;
   R^{c} is hydrogen or —(C₁-C₃)alkyl;
   q is 1, 2, 3, or 4; and
   [symbol] represents a covalent bond to the pyridone ring of formula (III) and [symbol] represents a covalent bond to the phenyl ring of formula (III).

The invention also relates to a compound of formula (IV):

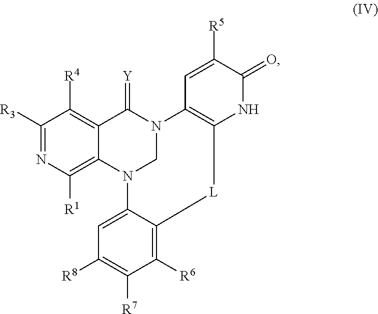

or a tautomer thereof, or a pharmaceutically acceptable salt thereof,
wherein:
   Y is O or S;
   each of R¹, R³ and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, —NHCH₂CH₂NHCH₂—, —NHCH₂CH₂OCH₂—, a divalent linker of formula (L-ia), or a divalent linker of formula (L-iia):

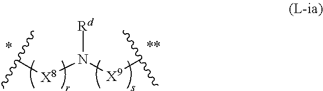

wherein:
   each of X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to the pyridone ring of formula (IV) and [symbol] represents a covalent bond to the phenyl ring of formula (IV);

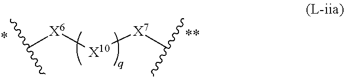

wherein:
   X⁶ is —NR— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl;
   R^{c} is hydrogen or —(C₁-C₃)alkyl;
   q is 1, 2, 3, or 4;
   and
   [symbol] represents a covalent bond to pyridone ring of formula (IV) and [symbol] represents a covalent bond to the phenyl ring of formula (IV).

The invention also relates to a compound of formula (IV):

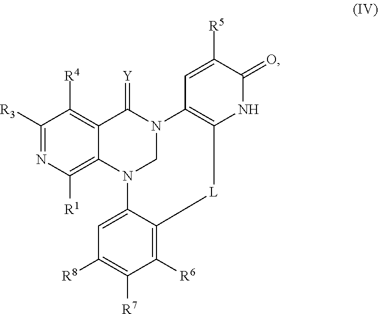

or a tautomer thereof, or a pharmaceutically acceptable salt thereof,
wherein:
   Y is O or S;
   each of R¹, R³ and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, a divalent linker of formula (L-i), or a divalent linker of formula (L-ii):

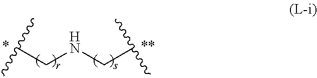

wherein:
   r is 1, 2, 3, or 4;
   s is 1, 2, 3, or 4;
   the sum of r and s is 2, 3, 4, or 5; and
   [symbol] represents a covalent bond to the pyridone ring of formula (IV) and [symbol] represents a covalent bond to the phenyl ring of formula (IV);

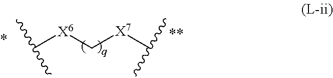

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof, Y is O. In another embodiment, Y is S.

In an embodiment of a compound of formula (IV), or a tautomer thereof, or a pharmaceutically acceptable salt thereof, each of R¹, R³, and R⁴ is independently hydrogen, halo, cyano, —(C₁-C₆)alkyl, —(C₁-C₆)alkoxy, halo(C₁-C₆)alkyl, or halo(C₁-C₆)alkoxy-. In another embodiment, each of R¹, R³, and R⁴ is independently hydrogen, halo, cyano, halo(C₁-C₆)alkyl, or halo(C₁-C₆)alkoxy-. In another embodiment, each of R¹, R³, and R⁴ is independently —F, —Cl, cyano, —CF₃, or —OCF₃.

In an embodiment of a compound of formula (IV), or a tautomer thereof, or a pharmaceutically acceptable salt thereof, R¹ is hydrogen and each of R³ and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-. In another embodiment, R¹ is hydrogen and each of R³ and R⁴ is independently hydrogen, halo, cyano, halo(C₁-C₆)alkyl, or halo(C₁-C₆)alkoxy-. In another embodiment, R¹ is hydrogen and each of R³ and R⁴ is independently —F, —Cl, cyano, —CF₃, or —OCF₃.

In an embodiment of a compound of formula (IV), or a tautomer thereof, or a pharmaceutically acceptable salt thereof, each of R^{a} and R^{b} is independently hydrogen or —(C₁-C₆)alkyl. In another embodiment, each of R and R is independently hydrogen or —CH₃.

In an embodiment of a compound of formula (IV), or a tautomer thereof, or a pharmaceutically acceptable salt thereof, R⁵ is hydrogen or —(C₁-C₆)alkyl. In another embodiment, R⁵ is hydrogen, —I, Cl, or —CH₃. In another embodiment, R⁵ is hydrogen.

In an embodiment of a compound of formula (IV), or a tautomer thereof, or a pharmaceutically acceptable salt thereof, each of R⁶, R⁷ and R⁸ is independently hydrogen, halo, halo(C₁-C₆)alkyl-, or halo(C₁-C₆)alkoxy-. In another embodiment, each of R⁶, R⁷ and R⁸ is independently hydrogen, —F, —CF₃, or —OCF₃.

In an embodiment of a compound of formula (IV), or a tautomer thereof, or a pharmaceutically acceptable salt thereof, R⁸ is hydrogen and each of R⁶ and R⁷ is independently hydrogen, halo, —(C₁-C₆)alkyl, —(C₁-C₆)alkoxy, halo(C₁-C₆)alkyl-, or halo(C₁-C₆)alkoxy-. In another embodiment, R⁸ is hydrogen and each of R⁶ and R⁷ is independently hydrogen, halo, halo(C₁-C₆)alkyl-, or halo(C₁-C₆)alkoxy-. In another embodiment, R⁸ is hydrogen and each of R⁶ and R⁷ is independently hydrogen, —F, —CF₃, or —OCF₃.

In an embodiment of a compound of formula (IV), or a tautomer thereof, or a pharmaceutically acceptable salt thereof, each of R⁶ and R⁸ is hydrogen, and R⁷ is hydrogen, halo, —(C₁-C₆)alkyl, —(C₁-C₆)alkoxy, halo(C₁-C₆)alkyl-, or halo(C₁-C₆)alkoxy-. In another embodiment, each of R⁶ and R⁸ is hydrogen, and R⁷ is hydrogen, halo, halo(C₁-C₆)alkyl-, or halo(C₁-C₆)alkoxy-. In another embodiment, each of R⁶ and R⁸ is hydrogen, and R⁷ is hydrogen, —F, —CF₃, or —OCF₃. In another embodiment, each of R⁶ and R⁸ is hydrogen, and R⁷ is —F, —CF₃, or —OCF₃. In another embodiment, each of R⁶ and R⁸ is hydrogen, and R⁷ is —F.

In an embodiment of a compound of formula (IV), or a tautomer thereof, or a pharmaceutically acceptable salt thereof, L is a divalent linker of 3 to 6 atoms in length, such as 3, 4, 5, or 6 atoms in length. In some embodiments, L is a divalent linker of 4 to 5 atoms in length. In some embodiments, L is a divalent linker of 4 atoms in length. In some embodiments, L is a divalent linker of 5 atoms in length.

In an embodiment of a compound of formula (IV), or a tautomer thereof, or a pharmaceutically acceptable salt thereof, L is (C₃-C₆)alkenylene, such as a C₃-alkenylene, C₄-alkenylene, C₅-alkenylene or C₆-alkenylene. In another embodiment, L is (C₃-C₆)alkenylene having one carbon-carbon double bond. In another embodiment, L is (C₄-C₆)alkenylene. In another embodiment, L is (C₄-C₆)alkenylene having one carbon-carbon double bond. In another embodiment, L is (C₄-C₅)alkenylene. In another embodiment, L is (C₄-C₅)alkenylene having one carbon-carbon double bond. In some embodiments, when L is a (C₃-C₆)alkenylene having one carbon-carbon double bond, the carbon-carbon double bond is in the cis configuration, trans configuration, or a mixture thereof, such as a mixture of cis:trans of 2:1 to 1:2, e.g., 2:1, 1:1, or 1:2. In another embodiment, L is (C₃-C₆)alkenylene selected from the group consisting of *—CH═CH—CH₂—**, *—CH₂—CH═CH—CH₂—**, *—CH₂CH₂—CH═CH—CH₂—**, and *—CH₂—CH═CH-CH₂CH₂CH₂—**, wherein “*” represents a covalent bond to the pyridone ring of formula (IV) and “**” represents a covalent bond to the phenyl ring of formula (IV). In another embodiment, L is (C₃-C₆)alkenylene selected from the group consisting of:

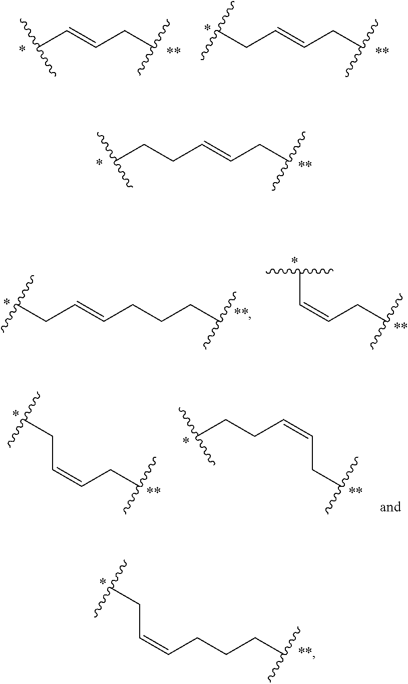

wherein [symbol] represents a covalent bond to the pyridone ring of formula (IV) and [symbol] represents a covalent bond to the phenyl ring of formula (IV).

In an embodiment of a compound of formula (I), or a tautomer thereof, or a pharmaceutically acceptable salt thereof, L is a divalent linker of formula (L-ia):

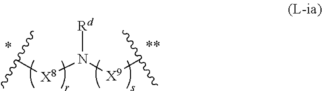

wherein:
   each of X⁸ and X⁹ is independently —CR⁹R¹⁰—, wherein each of R⁹ and R¹⁰ is independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
[symbol] represents a covalent bond to the pyridone ring of formula (IV) and [symbol] represents a covalent bond to the phenyl ring of formula (IV).
   In another embodiment, L is a divalent linker of formula (L-ia), wherein the sum of r and s is 3 or 4. In another embodiment, L is a divalent linker of formula (L-ia), wherein each of R⁹ and R¹⁰ is independently hydrogen, —CH₃, or —CH₂CH₃; and R^{d} is hydrogen or —CH₃. In another embodiment, L is a divalent linker of formula (L-ia) selected from the group consisting of:

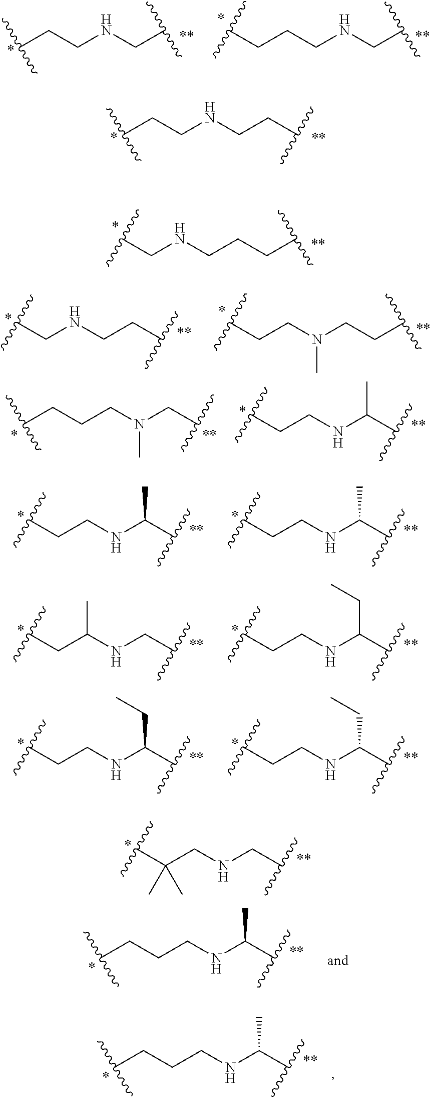

wherein [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof, L is a divalent linker of formula (L-i):

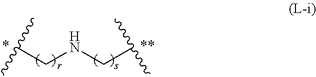

wherein:
   r is 1, 2, 3, or 4;
   s is 1, 2, 3, or 4;
   the sum of r and s is 2, 3, 4 or 5; and
   [symbol] represents a covalent bond to the pyridone ring of formula (IV) and [symbol] represents a covalent bond to the phenyl ring of formula (IV).
      In another embodiment, L is a divalent linker of formula (L-i), wherein the sum of r and s is 3 or 4. In another embodiment, L is a divalent linker of formula (L-i) selected from the group consisting of:

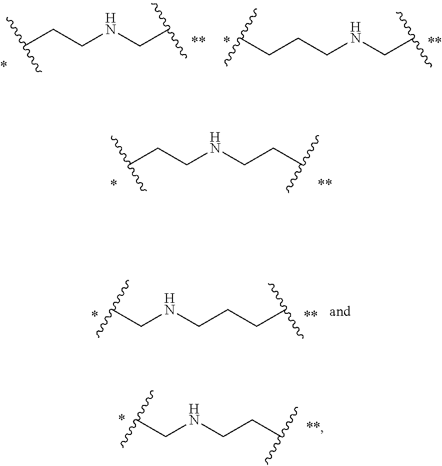

wherein [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof, L is a divalent linker of formula (L-iia):

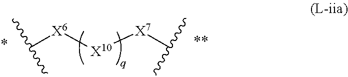

wherein:
   X⁶ is —NR— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴—, wherein each of R¹³ and R¹⁴ is independently hydrogen or —(C₁-C₃)alkyl;
   R^{c} is hydrogen or —(C₁-C₃)alkyl;
   q is 1, 2, 3, or 4; and
   [symbol] represents a covalent bond to the pyridone ring of formula (IV) and [symbol] represents a covalent bond to the phenyl ring of formula (IV).
      In another embodiment, L is a divalent linker of formula (L-iia), wherein X⁶ is —NR—; X⁷ is —CR¹¹R¹²—; R is hydrogen or —CH₃; and each of R¹¹ and R¹² is independently hydrogen or —CH₃.
      In another embodiment, L is a divalent linker of formula (L-iia) selected from the group consisting of:

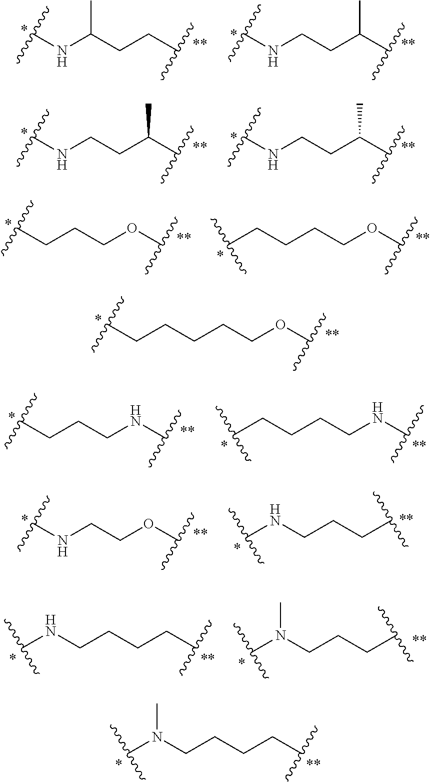

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof, L is a divalent linker of formula (L-ii):

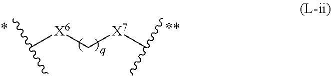

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (IV) and [symbol] represents a covalent bond to the phenyl ring of formula (IV).
      In another embodiment, L is a divalent linker of formula (L-ii), wherein R^{c} is hydrogen or —CH₃. In another embodiment, L is a divalent linker of formula (L-ii), wherein X⁶ is —NR^{c}— and X⁷ is —CH₂—. In another embodiment, L is a divalent linker of formula (L-ii), wherein X⁶ is —NR^{c} and X⁷ is —O—. In another embodiment, L is a divalent linker of formula (L-ii), wherein X⁶ is —CH₂— and X⁷ is —O—. In another embodiment, L is a divalent linker of formula (L-ii), wherein X⁶ is —CH₂— and X⁷ is —NH₂—. In another embodiment, L is a divalent linker of formula (L-ii), wherein X⁶ is —CH₂— and X⁷ is —CH₂—. In another embodiment, L is a divalent linker of formula (L-ii), wherein q is 2 or 3. In another embodiment, L is a divalent linker of formula (L-ii), wherein q is 2. In another embodiment, L is a divalent linker of formula (L-ii) selected from the group consisting of:

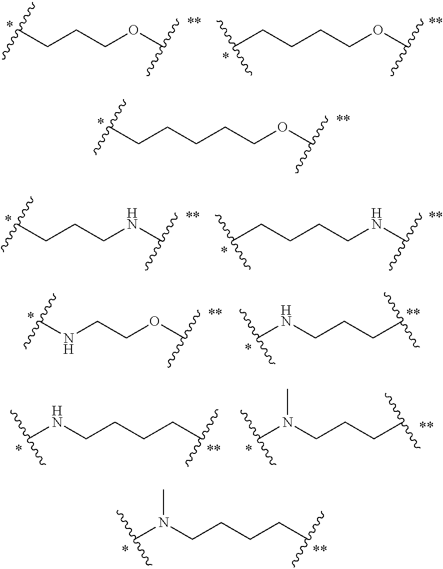

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (IV) and [symbol] represents a covalent bond to the phenyl ring of formula (IV). In another embodiment, L is a divalent linker of formula (L-ii) selected from the group consisting of: —CH₂CH₂CH₂—, —CH₂CH₂CH₂CH₂—, —CH₂CH₂CH₂CH₂CH₂—, and —CH₂CH₂CH₂CH₂CH₂CH₂—. In another embodiment, L is a divalent linker of formula (L-ii) selected from the group consisting of:

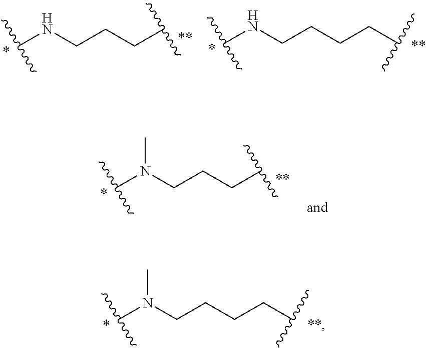

wherein [symbol] represents a covalent bond to the pyridone ring of formula (IV) and [symbol] represents a covalent bond to the phenyl ring of formula (IV). In another embodiment, L is a divalent linker of formula (L-ii) selected from the group consisting of:

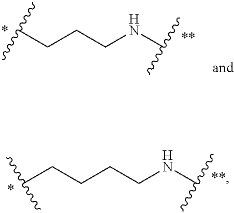

wherein [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof, L is a divalent linker selected from the group consisting of

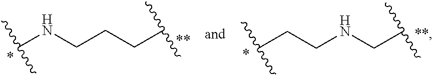

wherein [symbol] represents a covalent bond to the pyridone ring of formula (IV) and [symbol] represents a covalent bond to the phenyl ring of formula (IV). In another embodiment, L is the divalent linker

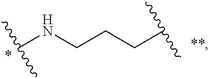

wherein [symbol] represents a covalent bond to the pyridone ring of formula (IV) and [symbol] represents a covalent bond to the phenyl ring of formula (IV). In another embodiment, L is the divalent linker

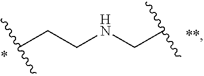

wherein [symbol] represents a covalent bond to the pyridone ring of formula (IV) and [symbol] represents a covalent bond to the phenyl ring of formula (IV).

In an embodiment of a compound of formula (IV), or a tautomer thereof, or a pharmaceutically acceptable salt thereof:
Y is O;
R¹ is hydrogen and each of R³ and R⁴ is independently hydrogen, halo, cyano, halo(C₁-C₆)alkyl-, or halo(C₁-C₆)alkoxy-;
R⁵ is hydrogen;
each of R⁶ and R⁷ is independently hydrogen, halo, halo(C₁-C₆)alkyl-, or halo(C₁-C₆)alkoxy-;
R⁸ is hydrogen; and
L is a divalent linker of formula (L-i) or a divalent linker of formula (L-ii):

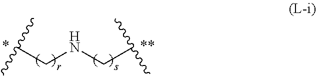

wherein:
   r is 1, 2, 3, or 4;
   s is 1, 2, 3, or 4;
   the sum of r and s is 2, 3, 4, or 5; and
   [symbol] represents a covalent bond to ring A of formula (IV) and [symbol] represents a covalent bond to the phenyl ring of formula (IV);

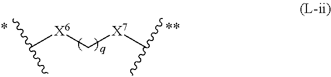

wherein:
   X⁶ is —NR—;
   X⁷ is —CH₂—;
   R^{c} is hydrogen or —(C₁-C₃)alkyl;
   q is 1, 2, 3, or 4; and
   [symbol] represents a covalent bond to the pyridone ring of formula (IV) and [symbol] represents a covalent bond to the phenyl ring of formula (IV).

The invention also relates to a compound of formula (V):

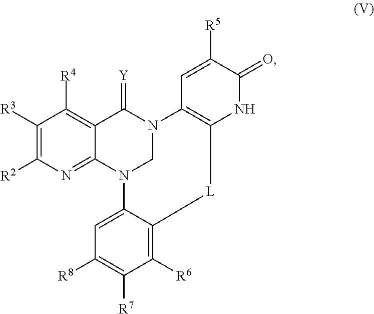

or a tautomer thereof, or a pharmaceutically acceptable salt thereof,
wherein:
   Y is O or S;
   each of R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, —NHCH₂CH₂NHCH₂—, —NHCH₂CH₂OCH₂—, a divalent linker of formula (L-ia), or a divalent linker of formula (L-iia):

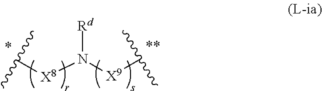

wherein:
   each of X⁸ and X⁹ is independently —CR⁹R¹⁰—, wherein each of R⁹ and R¹⁰ is each independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
   [symbol] represents a covalent bond to the pyridone ring of formula (V) and [symbol] represents a covalent bond to the phenyl ring of formula (V);

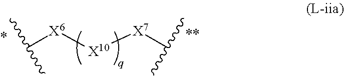

wherein:
   X⁶ is —NR^{c}— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴— wherein each of R¹³ and R¹⁴ is independently hydrogen or —(C₁-C₃)alkyl; R^{c} is hydrogen or —(C₁-C₃)alkyl;
   q is 1, 2, 3, or 4;
   and
   [symbol] represents a covalent bond to pyridone ring of formula (V) and [symbol] represents a covalent bond to the phenyl ring of formula (V).

The invention also relates to a compound of formula (V):

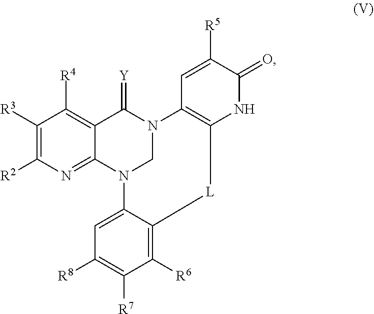

or a tautomer thereof, or a pharmaceutically acceptable salt thereof,
wherein:
   Y is O or S;
   each of R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
   R⁵ is hydrogen, halo, or —(C₁-C₆)alkyl;
   each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
   each of R^{a} and R^{b} is independently hydrogen, —(C₁-C₆)alkyl, or halo(C₁-C₆)alkyl-; and
   L is (C₃-C₆)alkenylene, a divalent linker of formula (L-i), or a divalent linker of formula (L-ii):

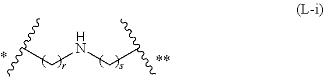

wherein:
   r is 1, 2, 3, or 4;
   s is 1, 2, 3, or 4;
   the sum of r and s is 2, 3, 4, or 5; and
   [symbol] represents a covalent bond to the pyridone ring of formula (V) and [symbol] represents a covalent bond to the phenyl ring of formula (V);

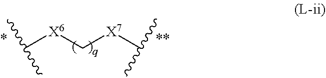

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof, Y is O. In another embodiment, Y is S.

In an embodiment of a compound of formula (V), or a tautomer thereof, or a pharmaceutically acceptable salt thereof, each of R², R³, and R⁴ is independently hydrogen, halo, cyano, —(C₁-C₆)alkyl, —(C₁-C₆)alkoxy, halo(C₁-C₆)alkyl, or halo(C₁-C₆)alkoxy-. In another embodiment, each of R², R³, and R⁴ is independently hydrogen, halo, cyano, halo(C₁-C₆)alkyl, or halo(C₁-C₆)alkoxy-. In another embodiment, each of R², R³, and R⁴ is independently —F, —Cl, cyano, —CF₃, or —OCF₃.

In an embodiment of a compound of formula (V), or a tautomer thereof, or a pharmaceutically acceptable salt thereof, each of R^{a} and R^{b} is independently hydrogen or —(C₁-C₆)alkyl. In another embodiment, each of R^{a} and R^{b} is independently hydrogen of —CH₃.

In an embodiment of a compound of formula (V), or a tautomer thereof, or a pharmaceutically acceptable salt thereof, R⁵ is hydrogen or —(C₁-C₆)alkyl. In another embodiment, R⁵ is hydrogen, —I, Cl, or —CH₃. In another embodiment, R⁵ is hydrogen.

In an embodiment of a compound of formula (V), or a tautomer thereof, or a pharmaceutically acceptable salt thereof, each of R⁶, R⁷ and R⁸ is independently hydrogen, halo, halo(C₁-C₆)alkyl-, or halo(C₁-C₆)alkoxy-. In another embodiment, each of R⁶, R⁷ and R⁸ is independently hydrogen, —F, —CF₃, or —OCF₃.

In an embodiment of a compound of formula (V), or a tautomer thereof, or a pharmaceutically acceptable salt thereof, R⁸ is hydrogen and each of R⁶ and R⁷ is independently hydrogen, halo, —(C₁-C₆)alkyl, —(C₁-C₆)alkoxy, halo(C₁-C₆)alkyl-, or halo(C₁-C₆)alkoxy-. In another embodiment, R⁸ is hydrogen and each of R⁶ and R⁷ is independently hydrogen, halo, halo(C₁-C₆)alkyl-, or halo(C₁-C₆)alkoxy-. In another embodiment, R⁸ is hydrogen and each of R⁶ and R⁷ is independently hydrogen, —F, —CF₃, or —OCF₃.

In an embodiment of a compound of formula (V), or a tautomer thereof, or a pharmaceutically acceptable salt thereof, each of R⁶ and R⁸ is hydrogen, and R⁷ is hydrogen, halo, —(C₁-C₆)alkyl, —(C₁-C₆)alkoxy, halo(C₁-C₆)alkyl-, or halo(C₁-C₆)alkoxy-. In another embodiment, each of R⁶ and R⁸ is hydrogen, and R⁷ is hydrogen, halo, halo(C₁-C₆)alkyl-, or halo(C₁-C₆)alkoxy-. In another embodiment, each of R⁶ and R⁸ is hydrogen, and R⁷ is hydrogen, —F, —CF₃, or —OCF₃. In another embodiment, each of R⁶ and R⁸ is hydrogen, and R⁷ is —F, —CF₃, or —OCF₃. In another embodiment, each of R⁶ and R⁸ is hydrogen, and R⁷ is —F.

In an embodiment of a compound of formula (V), or a tautomer thereof, or a pharmaceutically acceptable salt thereof, L is a divalent linker of 3 to 6 atoms in length, such as 3, 4, 5, or 6 atoms in length. In some embodiments, L is a divalent linker of 4 to 5 atoms in length. In some embodiments, L is a divalent linker of 4 atoms in length. In some embodiments, L is a divalent linker of 5 atoms in length.

In an embodiment of a compound of formula (V), or a tautomer thereof, or a pharmaceutically acceptable salt thereof, L is (C₃-C₆)alkenylene, such as a C₃-alkenylene, C₄-alkenylene, C₅-alkenylene or C₆-alkenylene. In another embodiment, L is (C₃-C₆)alkenylene having one carbon-carbon double bond. In another embodiment, L is (C₄-C₆)alkenylene. In another embodiment, L is (C₄-C₆)alkenylene having one carbon-carbon double bond. In another embodiment, L is (C₄-C₅)alkenylene. In another embodiment, L is (C₄-C₅)alkenylene having one carbon-carbon double bond. In some embodiments, when L is a (C₃-C₆)alkenylene having one carbon-carbon double bond, the carbon-carbon double bond is in the cis configuration, trans configuration, or a mixture thereof, such as a mixture of cis:trans of 2:1 to 1:2, e.g., 2:1, 1:1, or 1:2. In another embodiment, L is (C₃-C₆)alkenylene selected from the group consisting of *—CH═CH—CH₂—**, *—CH₂—CH═CH—CH₂—**, *—CH₂CH₂—CH═CH—CH₂—**, and *—CH₂—CH═CH—CH₂CH₂CH₂—**, wherein “*” represents a covalent bond to the pyridone ring of formula (V) and “**” represents a covalent bond to the phenyl ring of formula (V). In another embodiment, L is (C₃-C₆)alkenylene selected from the group consisting of:

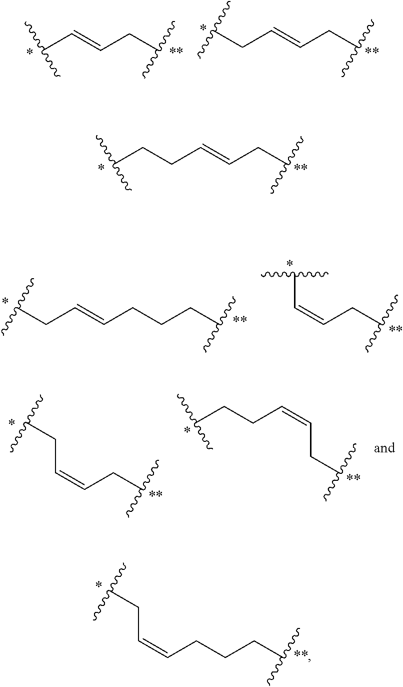

wherein [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof, L is a divalent linker of formula (L-ia):

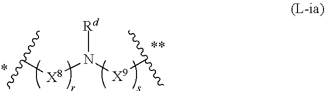

wherein:
   each of X⁸ and X⁹ is independently —CR⁹R¹⁰—, wherein each of R⁹ and R¹⁰ is independently hydrogen or —(C₁-C₃)alkyl;
   R^{d} is hydrogen or —(C₁-C₃)alkyl;
   r is 1, 2, 3, or 4;
   s is 1, 2, 3, or 4;
   the sum of r and s is 2, 3, 4, or 5; and
[symbol] represents a covalent bond to pyridone ring of formula (V) and [symbol] represents a covalent bond to the phenyl ring of formula (V).
   In another embodiment, L is a divalent linker of formula (L-ia), wherein the sum of r and s is 3 or 4. In another embodiment, L is a divalent linker of formula (L-ia), wherein each of R⁹ and R¹⁰ is independently hydrogen, —CH₃, or —CH₂CH₃; and R^{d} is hydrogen or —CH₃. In another embodiment, L is a divalent linker of formula (L-ia) selected from the group consisting of:

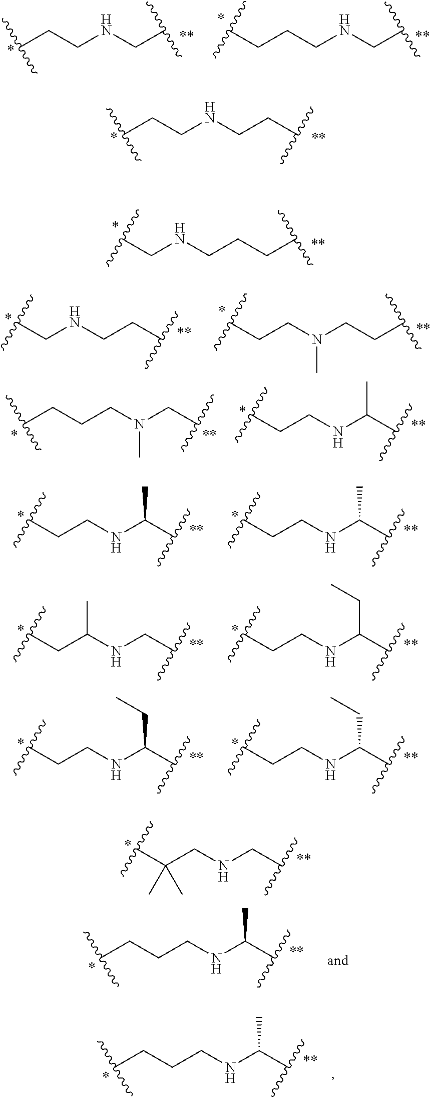

wherein [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof, L is a divalent linker of formula (L-i):

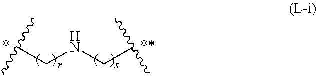

wherein:
   r is 1, 2, 3, or 4;
   s is 1, 2, 3, or 4;
   the sum of r and s is 2, 3, 4 or 5; and
[symbol] represents a covalent bond to the pyridone ring of formula (V) and [symbol] represents a covalent bond to the phenyl ring of formula (V).
   In another embodiment, L is a divalent linker of formula (L-i), wherein the sum of r and s is 3 or 4. In another embodiment, L is a divalent linker of formula (L-i) selected from the group consisting of:

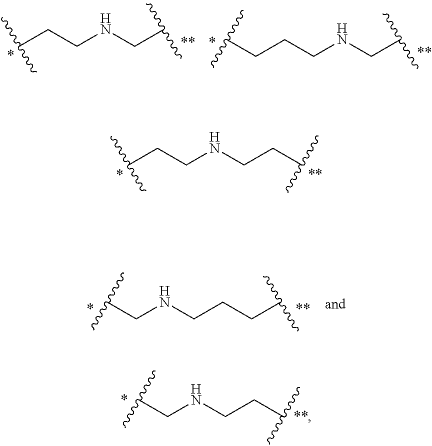

wherein [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof, L is a divalent linker of formula (L-iia):

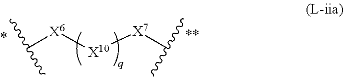

wherein:
   X⁶ is —NR— or —CH₂—;
   X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
   each X¹⁰ is independently —CR¹³R¹⁴—, wherein each of R¹³ and R¹⁴ is independently hydrogen or —(C₁-C₃)alkyl;
   R^{c} is hydrogen or —(C₁-C₃)alkyl;
   q is 1, 2, 3, or 4; and
   [symbol] represents a covalent bond to the pyridone ring of formula (V) and [symbol] represents a covalent bond to the phenyl ring of formula (V).
      In another embodiment, L is a divalent linker of formula (L-iia), wherein X⁶ is —NR—; X⁷ is —CR¹¹R¹²—; R is hydrogen or —CH₃; and each of R¹¹ and R¹² is independently hydrogen or —CH₃. In another embodiment, L is a divalent linker of formula (L-iia) selected from the group consisting of:

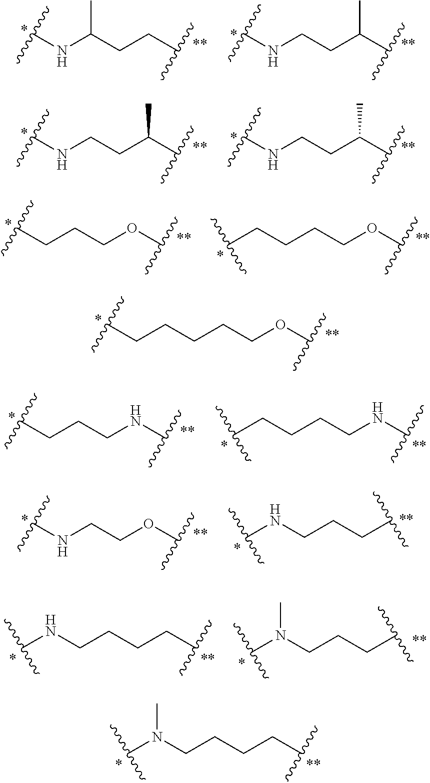

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the phenyl ring of formula (V) and [symbol] represents a covalent bond to the phenyl ring of formula (V).

In an embodiment of a compound of formula (V), or a tautomer thereof, or a pharmaceutically acceptable salt thereof, L is a divalent linker of formula (L-ii):

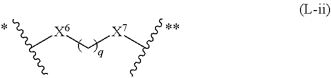

wherein:
   X⁶ is —NR— or —CH₂—;
   X⁷ is —CH₂—, —O— or —NH₂—, provided that when X⁷ is —NH₂—, X⁶ is —CH₂—;
   R^{c} is hydrogen or —(C₁-C₃)alkyl;
   q is 1, 2, 3, or 4; and
   [symbol] represents a covalent bond to the pyridone ring of formula (V) and [symbol] represents a covalent bond to the phenyl ring of formula (V).
      In another embodiment, L is a divalent linker of formula (L-ii), wherein R^{c} is hydrogen or —CH₃. In another embodiment, L is a divalent linker of formula (L-ii), wherein X⁶ is —NR^{c}— and X⁷ is —CH₂—. In another embodiment, L is a divalent linker of formula (L-ii), wherein X⁶ is —NR^{c} and X⁷ is —O—. In another embodiment, L is a divalent linker of formula (L-ii), wherein X⁶ is —CH₂— and X⁷ is —O—. In another embodiment, L is a divalent linker of formula (L-ii), wherein X⁶ is —CH₂— and X⁷ is —NH₂—. In another embodiment, L is a divalent linker of formula (L-ii), wherein X⁶ is —CH₂— and X⁷ is —CH₂—. In another embodiment, L is a divalent linker of formula (L-ii), wherein q is 2 or 3. In another embodiment, L is a divalent linker of formula (L-iii), wherein q is 2. In another embodiment, L is a divalent linker of formula (L-iii) selected from the group consisting of:

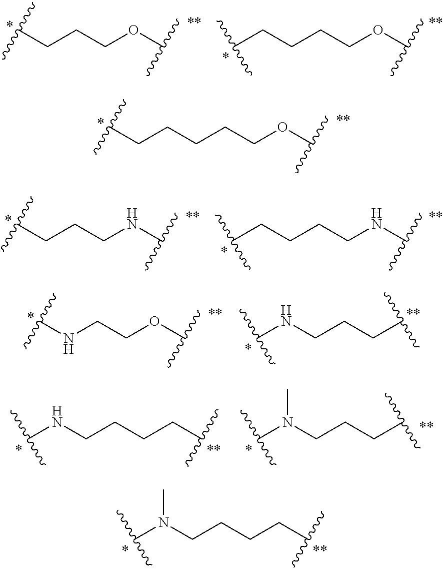

—CH₂CH₂CH₂—, —CH₂CH₂CH₂CH₂—, —CH₂CH₂CH₂CH₂CH₂—, and —CH₂CH₂CH₂CH₂CH₂CH₂—, wherein [symbol] represents a covalent bond to the pyridone ring of formula (V) and [symbol] represents a covalent bond to the phenyl ring of formula (V). In another embodiment, L is a divalent linker of formula (L-ii) selected from the group consisting of: —CH₂CH₂CH₂—, —CH₂CH₂CH₂CH₂—, —CH₂CH₂CH₂CH₂CH₂—, and —CH₂CH₂CH₂CH₂CH₂CH₂—. In another embodiment, L is a divalent linker of formula (L-ii) selected from the group consisting of:

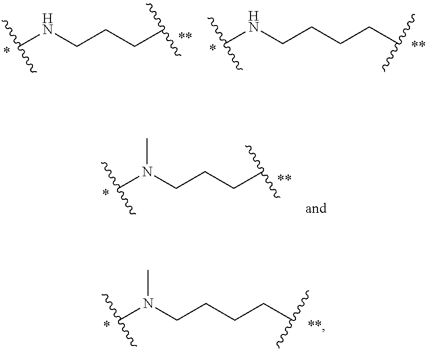

wherein [symbol] represents a covalent bond to the pyridone ring of formula (V) and [symbol] represents a covalent bond to the phenyl ring of formula (V). In another embodiment, L is a divalent linker of formula (L-ii) selected from the group consisting of:

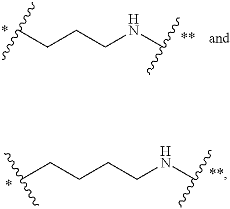

wherein [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof, L is a divalent linker selected from the group consisting of

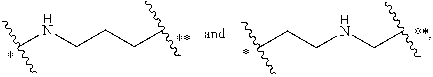

wherein [symbol] represents a covalent bond to the pyridone ring of formula (V) and [symbol] represents a covalent bond to the phenyl ring of formula (V). In another embodiment, L is the divalent linker

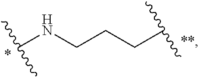

wherein [symbol] represents a covalent bond to the pyridone ring of formula (V) and [symbol] represents a covalent bond to the phenyl ring of formula (V). In another embodiment, L is the divalent linker

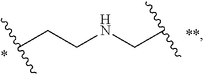

wherein [symbol] represents a covalent bond to the pyridone ring of formula (V) and [symbol] represents a covalent bond to the phenyl ring of formula (V).

In an embodiment of a compound of formula (V), or a tautomer thereof, or a pharmaceutically acceptable salt thereof:
Y is O;
each of R², R³ and R⁴ is independently hydrogen, halo, cyano, halo(C₁-C₆)alkyl-, or halo(C₁-C₆)alkoxy-;
R⁵ is hydrogen;
each of R⁶ and R⁷ is independently hydrogen, halo, halo(C₁-C₆)alkyl-, or halo(C₁-C₆)alkoxy-;
R⁵ is hydrogen; and
L is a divalent linker of formula (L-i) or a divalent linker of formula (L-ii):

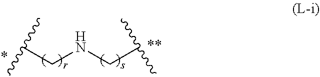

wherein:
   r is 1, 2, 3, or 4;
   s is 1, 2, 3, or 4;
   the sum of r and s is 2, 3, 4, or 5; and
   [symbol] represents a covalent bond to ring A of formula (V) and [symbol] represents a covalent bond to the phenyl ring of formula (V);

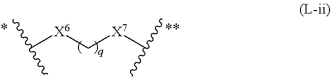

wherein:
   X⁶ is —NR^{c}—;
   X⁷ is —CH₂—;
   R^{c} is hydrogen or —(C₁-C₃)alkyl;
   q is 1, 2, 3, or 4; and
   [symbol] represents a covalent bond to the pyridone ring of formula (V) and [symbol] represents a covalent bond to the phenyl ring of formula (V).

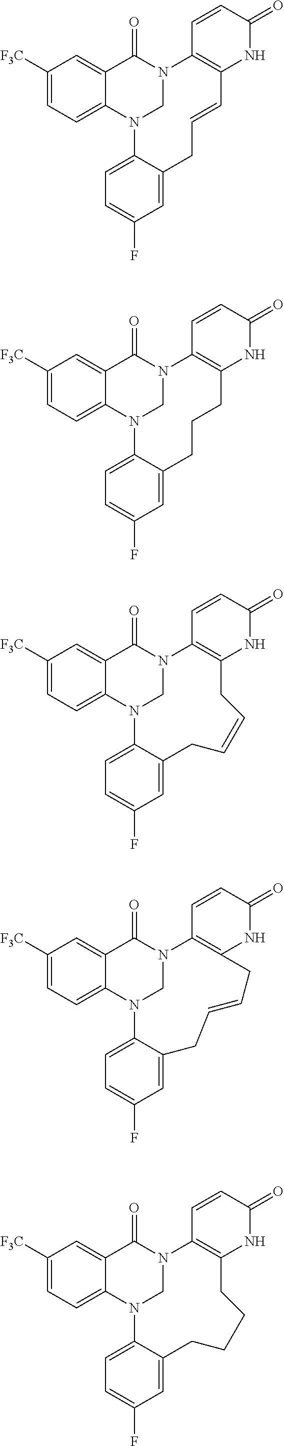 [SMILES: O=C1c2cc(C(F)(F)F)ccc2N2CN1c1ccc(=O)[nH]c1/C=C/Cc1cc(F)ccc12 ; O=C1c2cc(C(F)(F)F)ccc2N2CN1c1ccc(=O)[nH]c1CCCc1cc(F)ccc12 ; O=C1c2cc(C(F)(F)F)ccc2N2CN1c1ccc(=O)[nH]c1C/C=C\Cc1cc(F)ccc12 ; O=C1c2cc(C(F)(F)F)ccc2N2CN1c1ccc(=O)[nH]c1C/C=C/Cc1cc(F)ccc12 ; O=C1c2cc(C(F)(F)F)ccc2N2CN1c1ccc(=O)[nH]c1CCCCc1cc(F)ccc12]

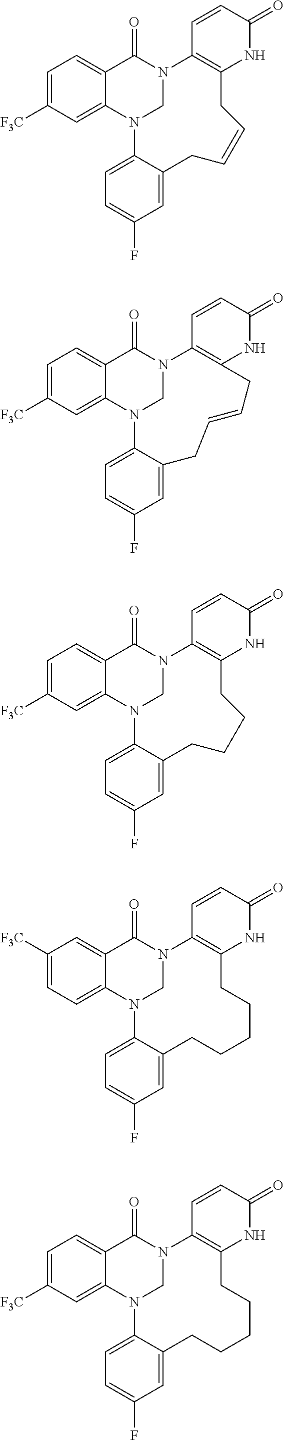 [SMILES: O=C1c2ccc(C(F)(F)F)cc2N2CN1c1ccc(=O)[nH]c1C/C=C\Cc1cc(F)ccc12 ; O=C1c2ccc(C(F)(F)F)cc2N2CN1c1ccc(=O)[nH]c1C/C=C/Cc1cc(F)ccc12 ; O=C1c2ccc(C(F)(F)F)cc2N2CN1c1ccc(=O)[nH]c1CCCCc1cc(F)ccc12 ; O=C1c2cc(C(F)(F)F)ccc2N2CN1c1ccc(=O)[nH]c1CCCCCc1cc(F)ccc12 ; O=C1c2ccc(C(F)(F)F)cc2N2CN1c1ccc(=O)[nH]c1CCCCCc1cc(F)ccc12]

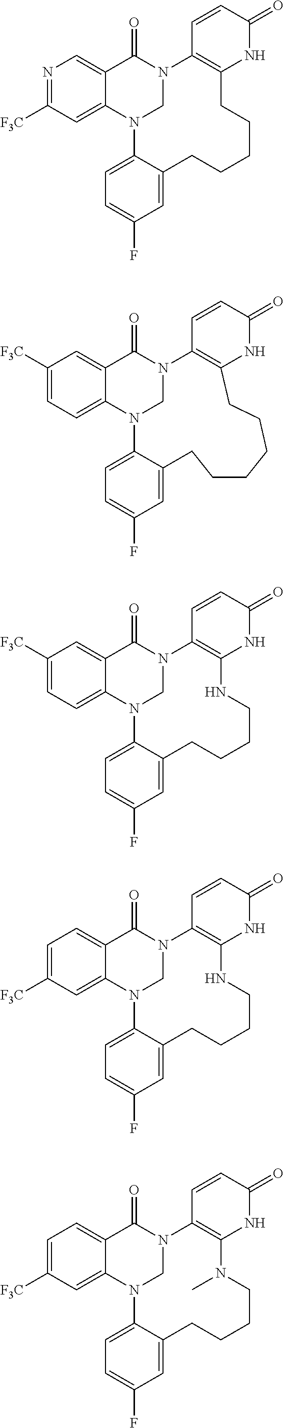 [SMILES: O=C1c2cnc(C(F)(F)F)cc2N2CN1c1ccc(=O)[nH]c1CCCCCc1cc(F)ccc12 ; O=C1c2cc(C(F)(F)F)ccc2N2CN1c1ccc(=O)[nH]c1CCCCCCc1cc(F)ccc12 ; O=C1c2cc(C(F)(F)F)ccc2N2CN1c1ccc(=O)[nH]c1NCCCCc1cc(F)ccc12 ; O=C1c2ccc(C(F)(F)F)cc2N2CN1c1ccc(=O)[nH]c1NCCCCc1cc(F)ccc12 ; CN1CCCCc2cc(F)ccc2N2CN(C(=O)c3ccc(C(F)(F)F)cc32)c2ccc(=O)[nH]c21]

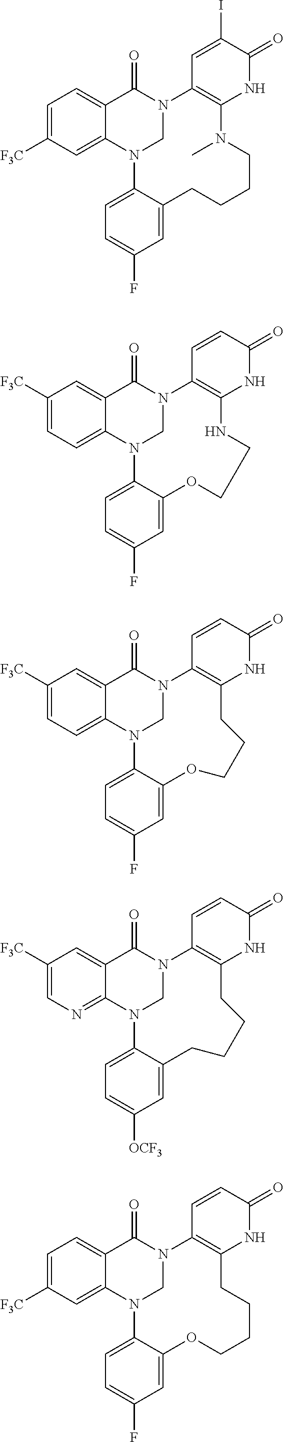

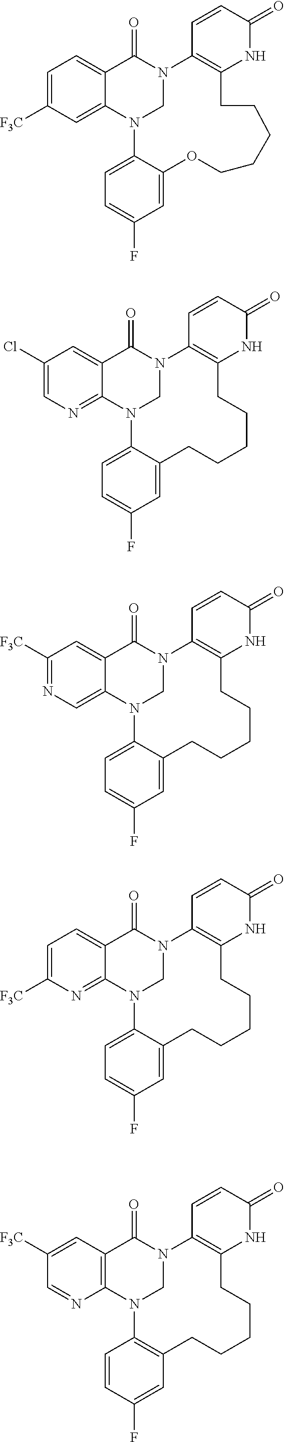 [SMILES: O=C1c2ccc(C(F)(F)F)cc2N2CN1c1ccc(=O)[nH]c1CCCCCOc1cc(F)ccc12 ; O=C1c2cc(Cl)cnc2N2CN1c1ccc(=O)[nH]c1CCCCCc1cc(F)ccc12 ; O=C1c2cc(C(F)(F)F)ncc2N2CN1c1ccc(=O)[nH]c1CCCCCc1cc(F)ccc12 ; O=C1c2ccc(C(F)(F)F)nc2N2CN1c1ccc(=O)[nH]c1CCCCCc1cc(F)ccc12 ; O=C1c2cc(C(F)(F)F)cnc2N2CN1c1ccc(=O)[nH]c1CCCCCc1cc(F)ccc12]

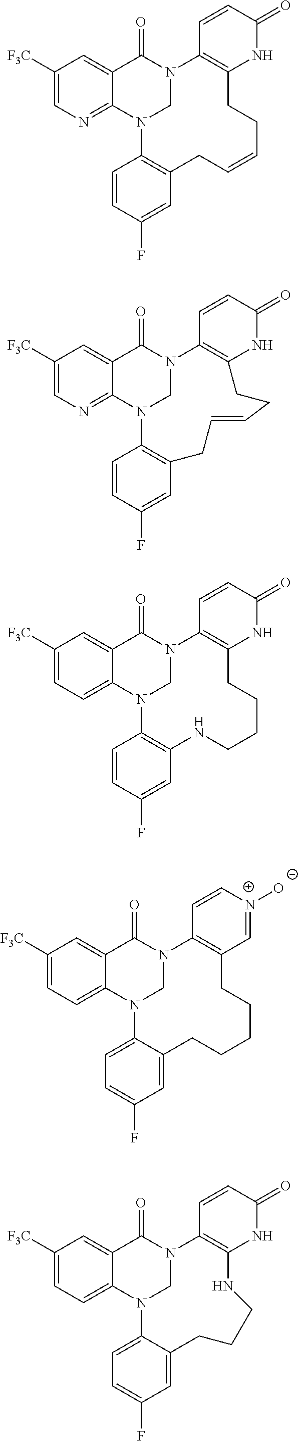 [SMILES: O=C1c2cc(C(F)(F)F)cnc2N2CN1c1ccc(=O)[nH]c1CC/C=C\Cc1cc(F)ccc12 ; O=C1c2cc(C(F)(F)F)cnc2N2CN1c1ccc(=O)[nH]c1CC/C=C/Cc1cc(F)ccc12 ; O=C1c2cc(C(F)(F)F)ccc2N2CN1c1ccc(=O)[nH]c1CCCCNc1cc(F)ccc12 ; O=C1c2cc(C(F)(F)F)ccc2N2CN1c1cc[n+]([O-])cc1CCCCCc1cc(F)ccc12 ; O=C1c2cc(C(F)(F)F)ccc2N2CN1c1ccc(=O)[nH]c1NCCCc1cc(F)ccc12]

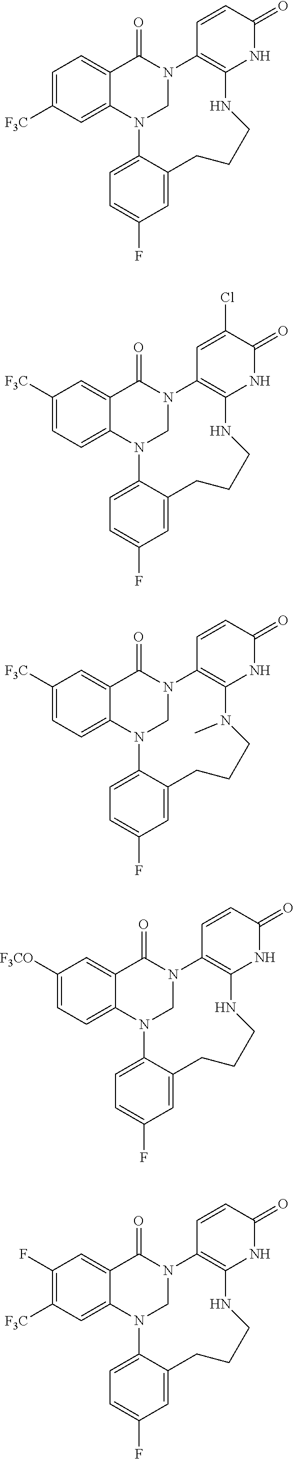 [SMILES: O=C1c2ccc(C(F)(F)F)cc2N2CN1c1ccc(=O)[nH]c1NCCCc1cc(F)ccc12 ; O=C1c2cc(C(F)(F)F)ccc2N2CN1c1cc(Cl)c(=O)[nH]c1NCCCc1cc(F)ccc12 ; CN1CCCc2cc(F)ccc2N2CN(C(=O)c3cc(C(F)(F)F)ccc32)c2ccc(=O)[nH]c21 ; O=C1c2cc(OC(F)(F)F)ccc2N2CN1c1ccc(=O)[nH]c1NCCCc1cc(F)ccc12 ; O=C1c2cc(F)c(C(F)(F)F)cc2N2CN1c1ccc(=O)[nH]c1NCCCc1cc(F)ccc12]

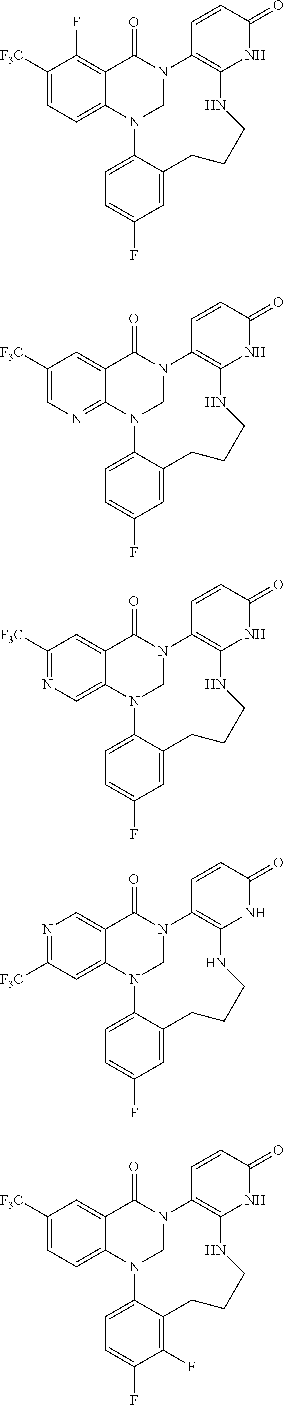

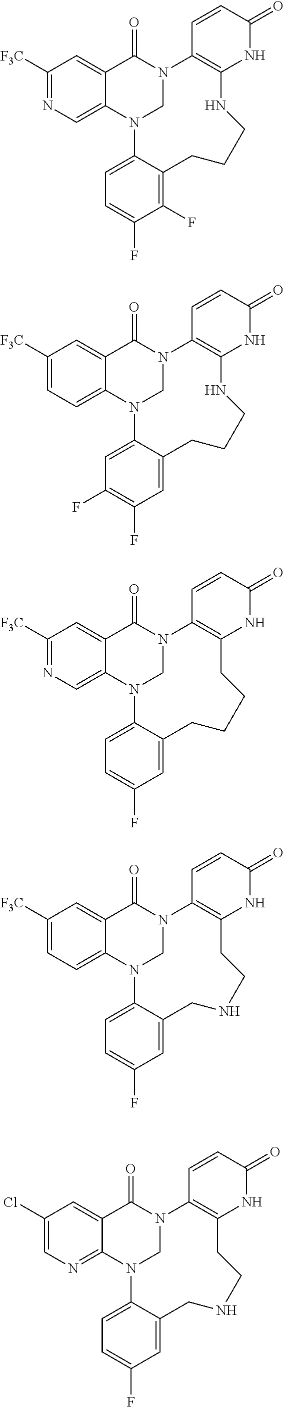 [SMILES: O=C1c2cc(C(F)(F)F)ncc2N2CN1c1ccc(=O)[nH]c1NCCCc1c2ccc(F)c1F ; O=C1c2cc(C(F)(F)F)ccc2N2CN1c1ccc(=O)[nH]c1NCCCc1cc(F)c(F)cc12 ; O=C1c2cc(C(F)(F)F)ncc2N2CN1c1ccc(=O)[nH]c1CCCCc1cc(F)ccc12 ; O=C1c2cc(C(F)(F)F)ccc2N2CN1c1ccc(=O)[nH]c1CCNCc1cc(F)ccc12 ; O=C1c2cc(Cl)cnc2N2CN1c1ccc(=O)[nH]c1CCNCc1cc(F)ccc12]

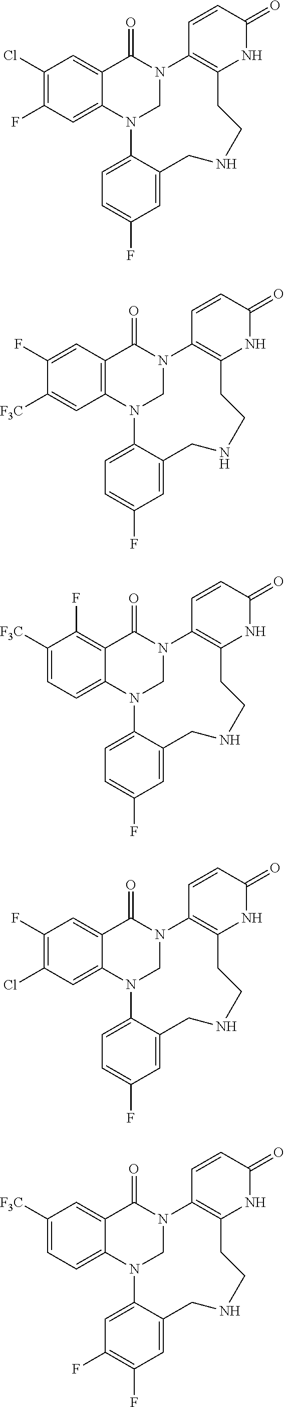 [SMILES: O=C1c2cc(Cl)c(F)cc2N2CN1c1ccc(=O)[nH]c1CCNCc1cc(F)ccc12 ; O=C1c2cc(F)c(C(F)(F)F)cc2N2CN1c1ccc(=O)[nH]c1CCNCc1cc(F)ccc12 ; O=C1c2c(ccc(C(F)(F)F)c2F)N2CN1c1ccc(=O)[nH]c1CCNCc1cc(F)ccc12 ; O=C1c2cc(F)c(Cl)cc2N2CN1c1ccc(=O)[nH]c1CCNCc1cc(F)ccc12 ; O=C1c2cc(C(F)(F)F)ccc2N2CN1c1ccc(=O)[nH]c1CCNCc1cc(F)c(F)cc12]

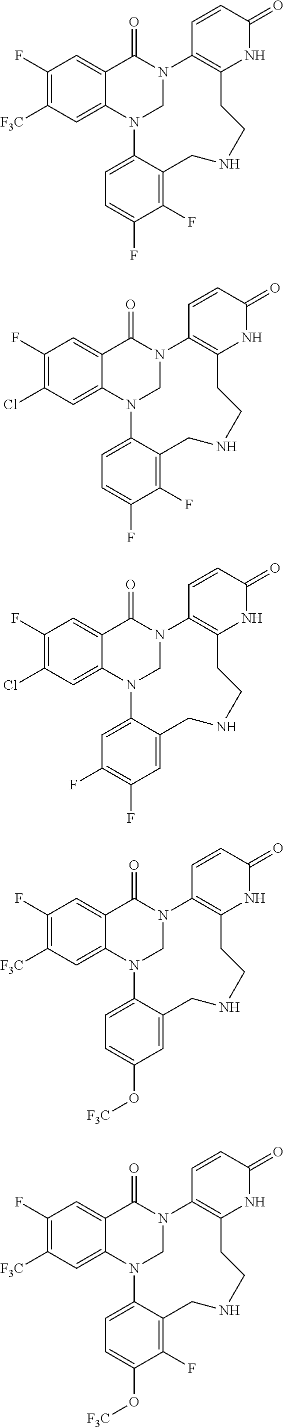 [SMILES: O=C1c2cc(F)c(C(F)(F)F)cc2N2CN1c1ccc(=O)[nH]c1CCNCc1c2ccc(F)c1F ; O=C1c2cc(F)c(Cl)cc2N2CN1c1ccc(=O)[nH]c1CCNCc1c2ccc(F)c1F ; O=C1c2cc(F)c(Cl)cc2N2CN1c1ccc(=O)[nH]c1CCNCc1cc(F)c(F)cc12 ; O=C1c2cc(F)c(C(F)(F)F)cc2N2CN1c1ccc(=O)[nH]c1CCNCc1cc(OC(F)(F)F)ccc12 ; O=C1c2cc(F)c(C(F)(F)F)cc2N2CN1c1ccc(=O)[nH]c1CCNCc1c2ccc(OC(F)(F)F)c1F]

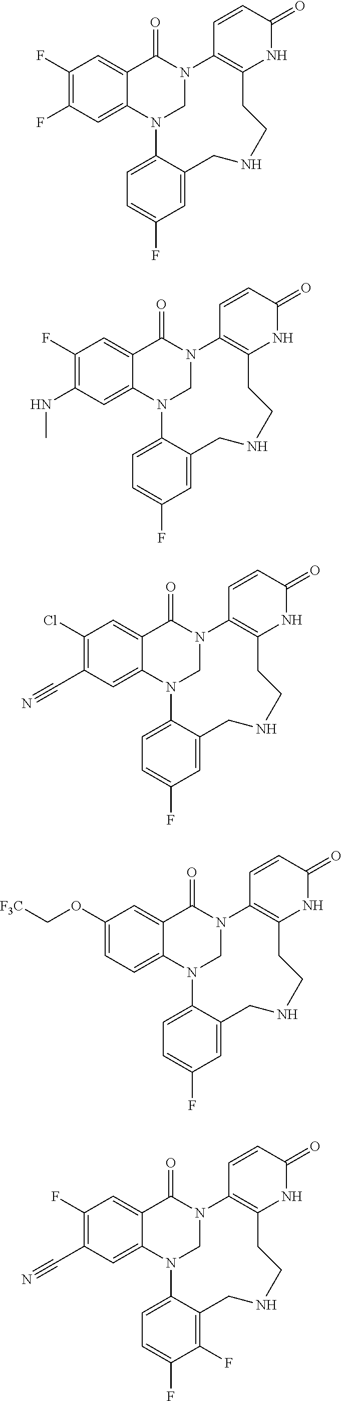 [SMILES: O=C1c2cc(F)c(F)cc2N2CN1c1ccc(=O)[nH]c1CCNCc1cc(F)ccc12 ; CNc1cc2c(cc1F)C(=O)N1CN2c2ccc(F)cc2CNCCc2[nH]c(=O)ccc21 ; N#Cc1cc2c(cc1Cl)C(=O)N1CN2c2ccc(F)cc2CNCCc2[nH]c(=O)ccc21 ; O=C1c2cc(OCC(F)(F)F)ccc2N2CN1c1ccc(=O)[nH]c1CCNCc1cc(F)ccc12 ; N#Cc1cc2c(cc1F)C(=O)N1CN2c2ccc(F)c(F)c2CNCCc2[nH]c(=O)ccc21]

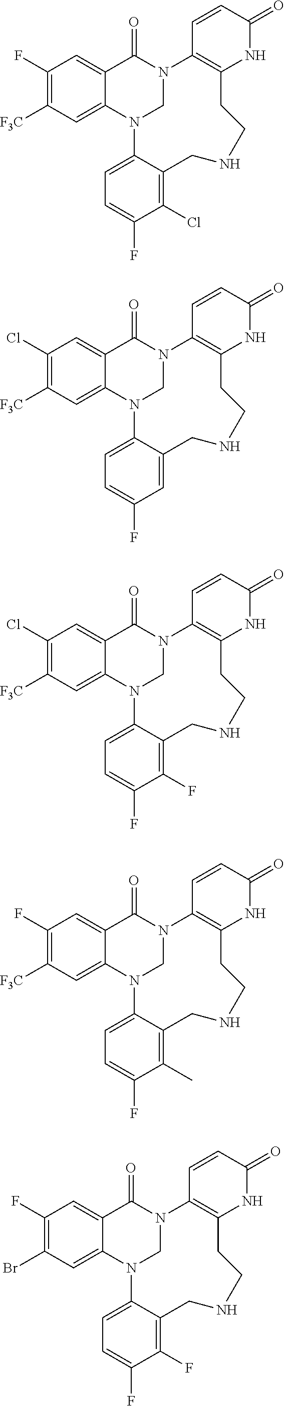 [SMILES: O=C1c2cc(F)c(C(F)(F)F)cc2N2CN1c1ccc(=O)[nH]c1CCNCc1c2ccc(F)c1Cl ; O=C1c2cc(Cl)c(C(F)(F)F)cc2N2CN1c1ccc(=O)[nH]c1CCNCc1cc(F)ccc12 ; O=C1c2cc(Cl)c(C(F)(F)F)cc2N2CN1c1ccc(=O)[nH]c1CCNCc1c2ccc(F)c1F ; Cc1c(F)ccc2c1CNCCc1[nH]c(=O)ccc1N1CN2c2cc(C(F)(F)F)c(F)cc2C1=O ; O=C1c2cc(F)c(Br)cc2N2CN1c1ccc(=O)[nH]c1CCNCc1c2ccc(F)c1F]

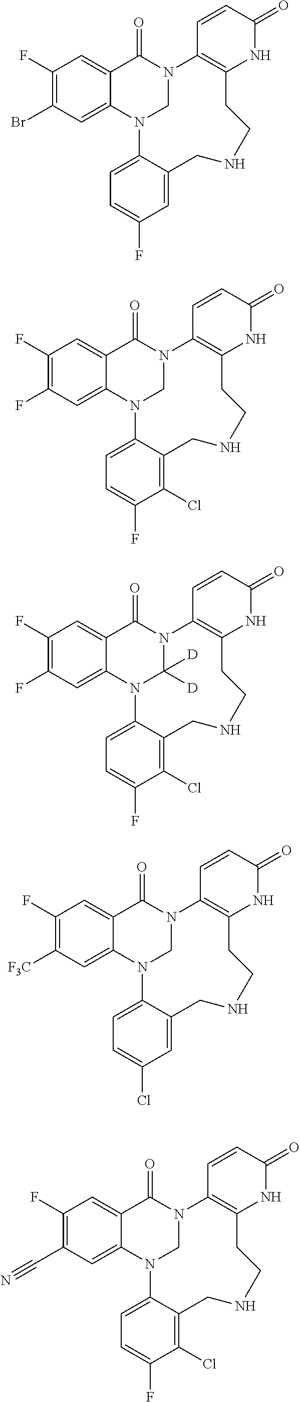

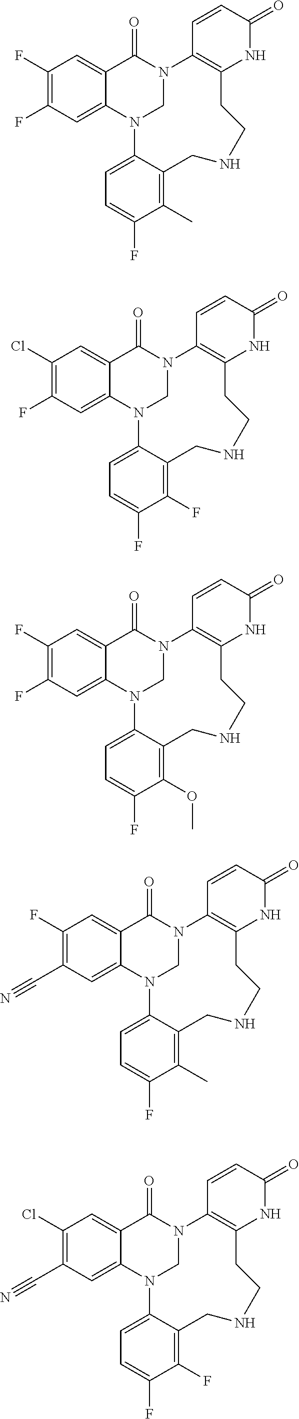 [SMILES: Cc1c(F)ccc2c1CNCCc1[nH]c(=O)ccc1N1CN2c2cc(F)c(F)cc2C1=O ; O=C1c2cc(Cl)c(F)cc2N2CN1c1ccc(=O)[nH]c1CCNCc1c2ccc(F)c1F ; COc1c(F)ccc2c1CNCCc1[nH]c(=O)ccc1N1CN2c2cc(F)c(F)cc2C1=O ; Cc1c(F)ccc2c1CNCCc1[nH]c(=O)ccc1N1CN2c2cc(C#N)c(F)cc2C1=O ; N#Cc1cc2c(cc1Cl)C(=O)N1CN2c2ccc(F)c(F)c2CNCCc2[nH]c(=O)ccc21]

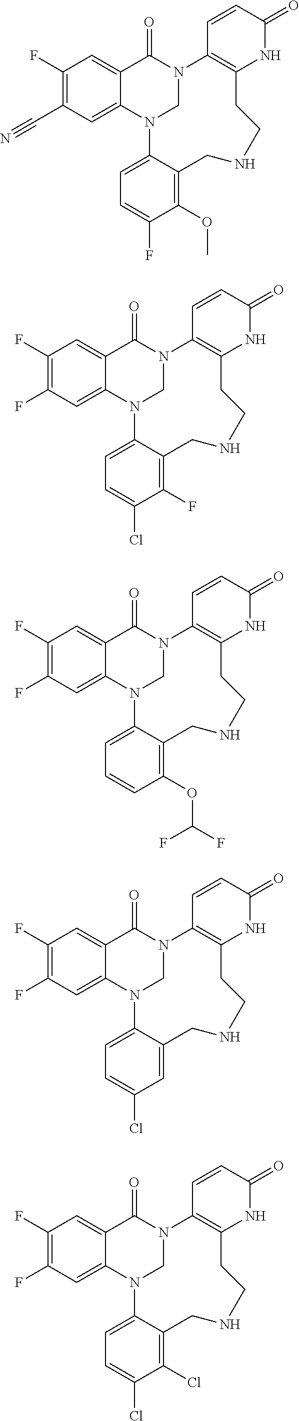

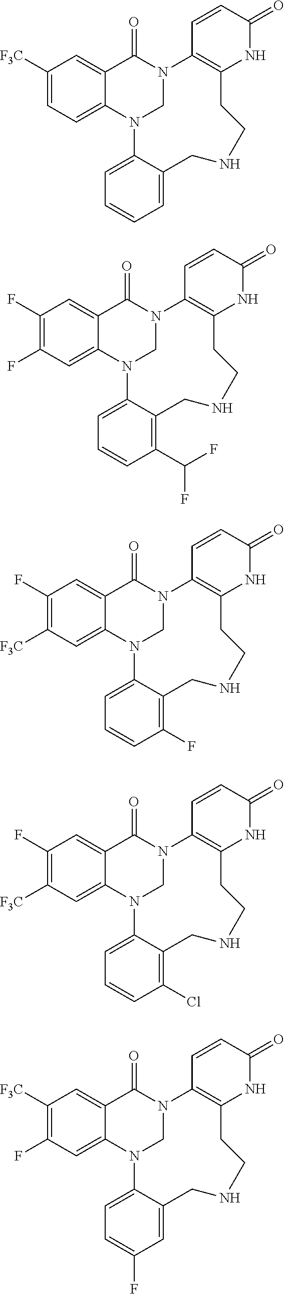

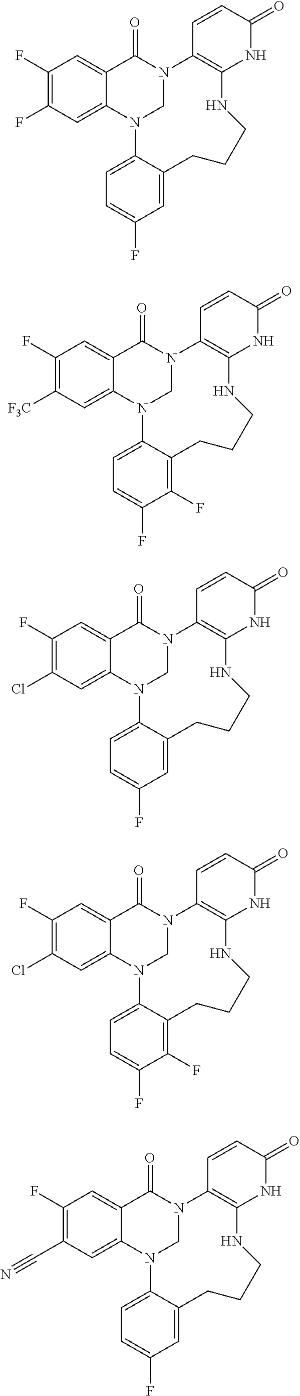 [SMILES: O=C1c2cc(F)c(F)cc2N2CN1c1ccc(=O)[nH]c1NCCCc1cc(F)ccc12 ; O=C1c2cc(F)c(C(F)(F)F)cc2N2CN1c1ccc(=O)[nH]c1NCCCc1c2ccc(F)c1F ; O=C1c2cc(F)c(Cl)cc2N2CN1c1ccc(=O)[nH]c1NCCCc1cc(F)ccc12 ; O=C1c2cc(F)c(Cl)cc2N2CN1c1ccc(=O)[nH]c1NCCCc1c2ccc(F)c1F ; N#Cc1cc2c(cc1F)C(=O)N1CN2c2ccc(F)cc2CCCNc2[nH]c(=O)ccc21]

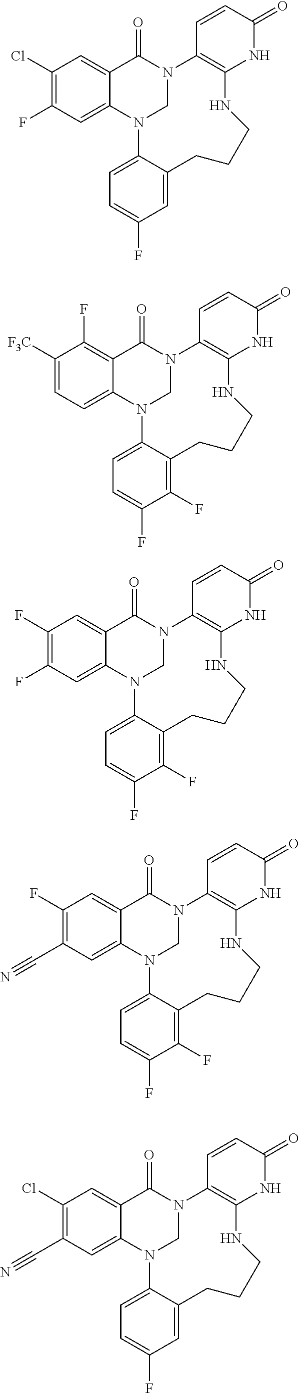

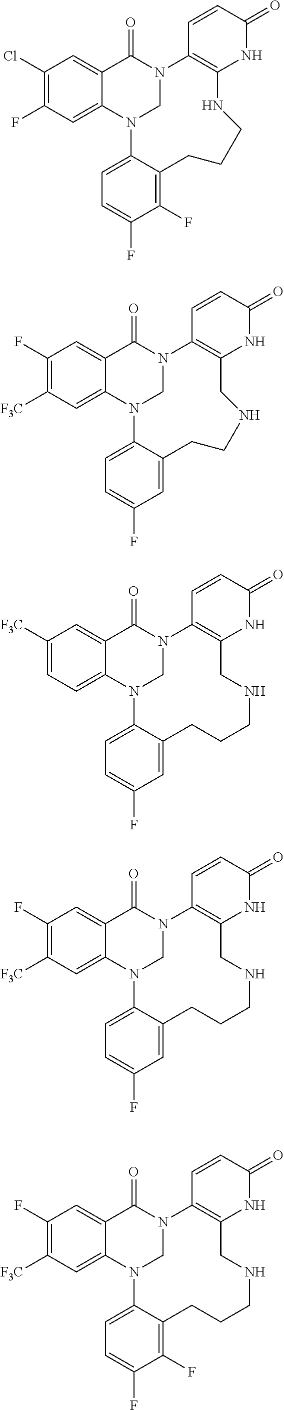 [SMILES: O=C1c2cc(Cl)c(F)cc2N2CN1c1ccc(=O)[nH]c1NCCCc1c2ccc(F)c1F ; O=C1c2cc(F)c(C(F)(F)F)cc2N2CN1c1ccc(=O)[nH]c1CNCCc1cc(F)ccc12 ; O=C1c2cc(C(F)(F)F)ccc2N2CN1c1ccc(=O)[nH]c1CNCCCc1cc(F)ccc12 ; O=C1c2cc(F)c(C(F)(F)F)cc2N2CN1c1ccc(=O)[nH]c1CNCCCc1cc(F)ccc12 ; O=C1c2cc(F)c(C(F)(F)F)cc2N2CN1c1ccc(=O)[nH]c1CNCCCc1c2ccc(F)c1F]

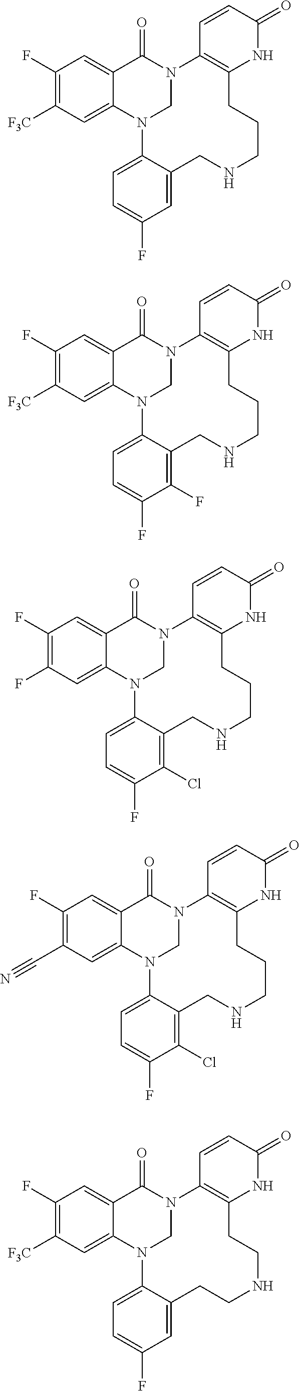

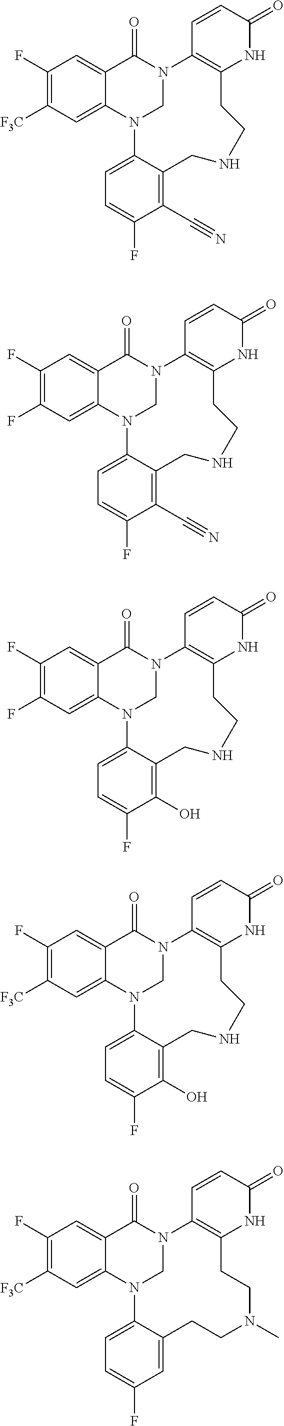 [SMILES: N#Cc1c(F)ccc2c1CNCCc1[nH]c(=O)ccc1N1CN2c2cc(C(F)(F)F)c(F)cc2C1=O ; N#Cc1c(F)ccc2c1CNCCc1[nH]c(=O)ccc1N1CN2c2cc(F)c(F)cc2C1=O ; O=C1c2cc(F)c(F)cc2N2CN1c1ccc(=O)[nH]c1CCNCc1c2ccc(F)c1O ; O=C1c2cc(F)c(C(F)(F)F)cc2N2CN1c1ccc(=O)[nH]c1CCNCc1c2ccc(F)c1O ; CN1CCc2cc(F)ccc2N2CN(C(=O)c3cc(F)c(C(F)(F)F)cc32)c2ccc(=O)[nH]c2CC1]

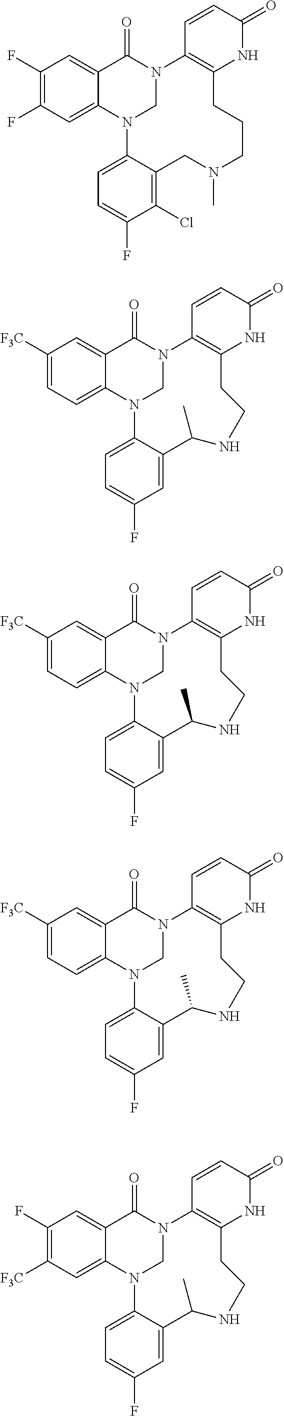 [SMILES: CN1CCCc2[nH]c(=O)ccc2N2CN(c3cc(F)c(F)cc3C2=O)c2ccc(F)c(Cl)c2C1 ; CC1NCCc2[nH]c(=O)ccc2N2CN(c3ccc(C(F)(F)F)cc3C2=O)c2ccc(F)cc21 ; C[C@H]1NCCc2[nH]c(=O)ccc2N2CN(c3ccc(C(F)(F)F)cc3C2=O)c2ccc(F)cc21 ; C[C@@H]1NCCc2[nH]c(=O)ccc2N2CN(c3ccc(C(F)(F)F)cc3C2=O)c2ccc(F)cc21 ; CC1NCCc2[nH]c(=O)ccc2N2CN(c3cc(C(F)(F)F)c(F)cc3C2=O)c2ccc(F)cc21]

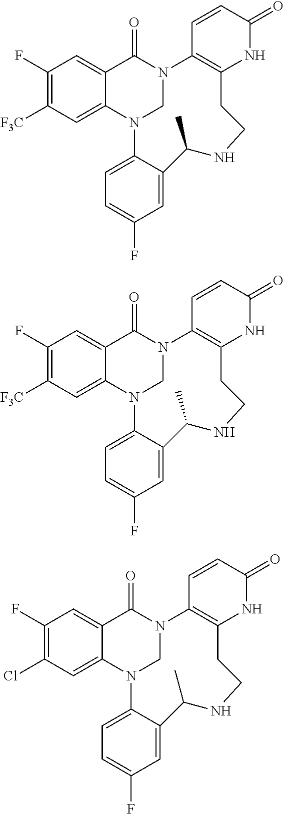

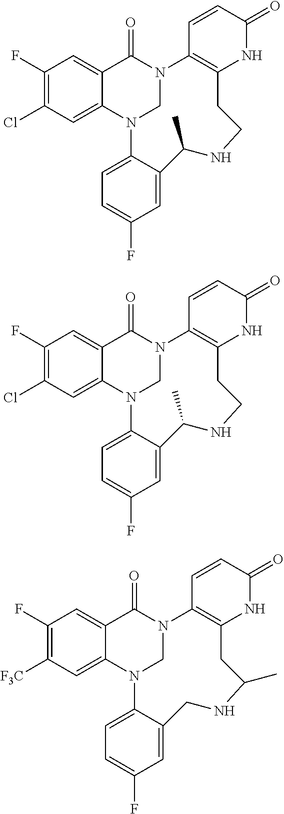

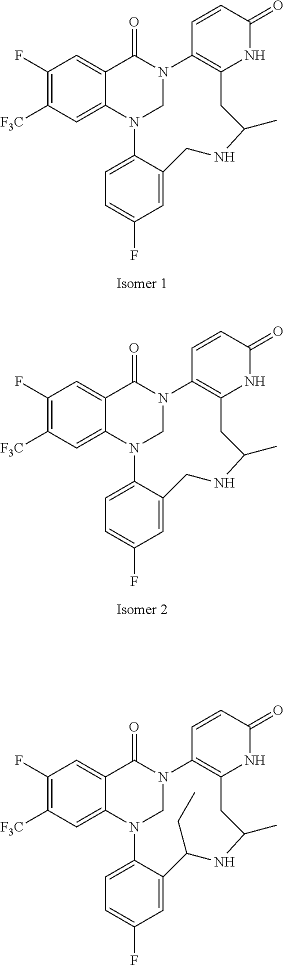 [SMILES: CC1Cc2[nH]c(=O)ccc2N2CN(c3ccc(F)cc3CN1)c1cc(C(F)(F)F)c(F)cc1C2=O ; CC1Cc2[nH]c(=O)ccc2N2CN(c3ccc(F)cc3CN1)c1cc(C(F)(F)F)c(F)cc1C2=O ; CCC1NC(C)Cc2[nH]c(=O)ccc2N2CN(c3cc(C(F)(F)F)c(F)cc3C2=O)c2ccc(F)cc21]

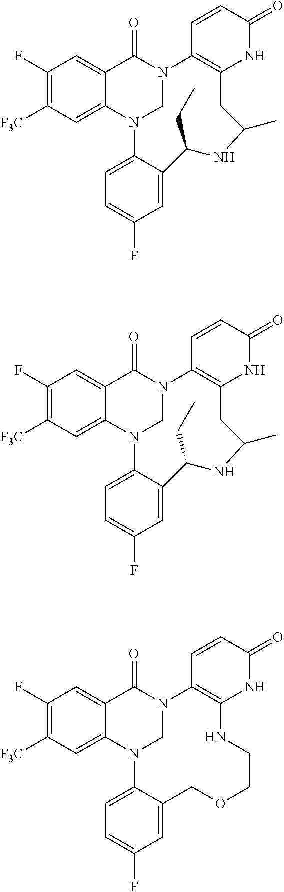 [SMILES: CC[C@H]1NC(C)Cc2[nH]c(=O)ccc2N2CN(c3cc(C(F)(F)F)c(F)cc3C2=O)c2ccc(F)cc21 ; CC[C@@H]1NC(C)Cc2[nH]c(=O)ccc2N2CN(c3cc(C(F)(F)F)c(F)cc3C2=O)c2ccc(F)cc21 ; O=C1c2cc(F)c(C(F)(F)F)cc2N2CN1c1ccc(=O)[nH]c1NCCOCc1cc(F)ccc12]

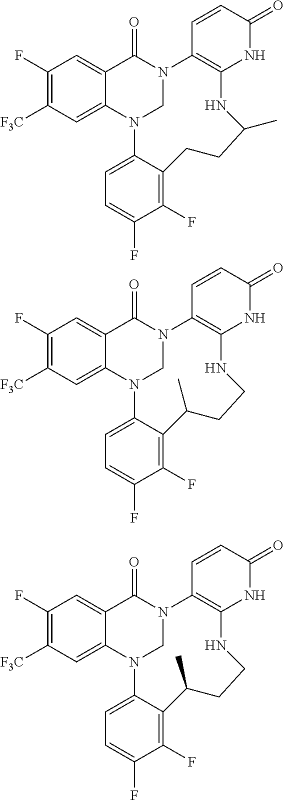

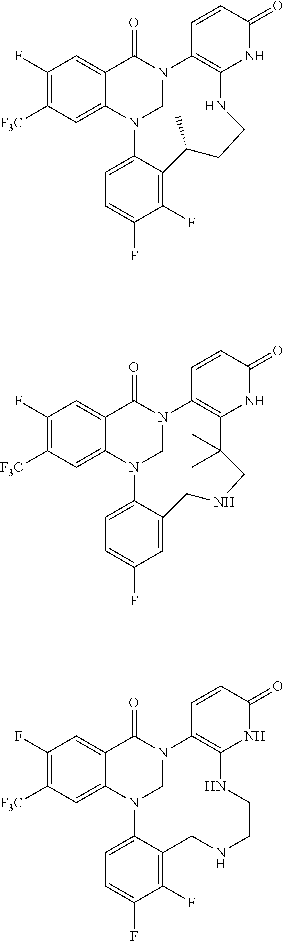 [SMILES: C[C@@H]1CCNc2[nH]c(=O)ccc2N2CN(c3cc(C(F)(F)F)c(F)cc3C2=O)c2ccc(F)c(F)c21 ; CC1(C)CNCc2cc(F)ccc2N2CN(C(=O)c3cc(F)c(C(F)(F)F)cc32)c2ccc(=O)[nH]c21 ; O=C1c2cc(F)c(C(F)(F)F)cc2N2CN1c1ccc(=O)[nH]c1NCCNCc1c2ccc(F)c1F]

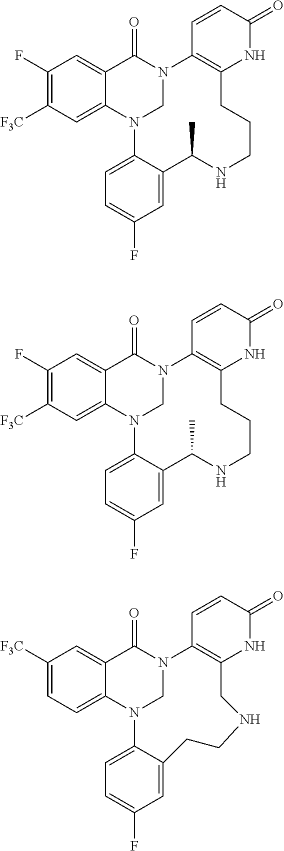 [SMILES: C[C@H]1NCCCc2[nH]c(=O)ccc2N2CN(c3cc(C(F)(F)F)c(F)cc3C2=O)c2ccc(F)cc21 ; C[C@@H]1NCCCc2[nH]c(=O)ccc2N2CN(c3cc(C(F)(F)F)c(F)cc3C2=O)c2ccc(F)cc21 ; O=C1c2cc(C(F)(F)F)ccc2N2CN1c1ccc(=O)[nH]c1CNCCc1cc(F)ccc12]

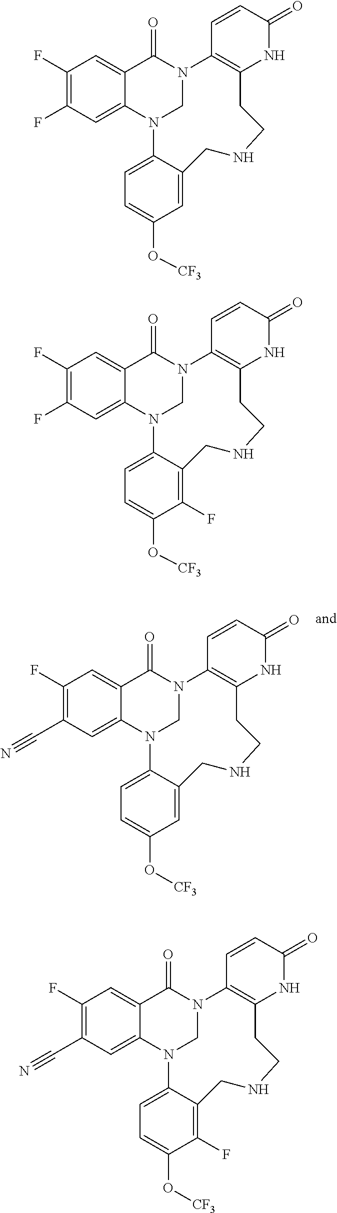 [SMILES: O=C1c2cc(F)c(F)cc2N2CN1c1ccc(=O)[nH]c1CCNCc1cc(OC(F)(F)F)ccc12 ; O=C1c2cc(F)c(F)cc2N2CN1c1ccc(=O)[nH]c1CCNCc1c2ccc(OC(F)(F)F)c1F ; N#Cc1cc2c(cc1F)C(=O)N1CN2c2ccc(OC(F)(F)F)cc2CNCCc2[nH]c(=O)ccc21 ; N#Cc1cc2c(cc1F)C(=O)N1CN2c2ccc(OC(F)(F)F)c(F)c2CNCCc2[nH]c(=O)ccc21]

or a tautomer thereof, or a pharmaceutically acceptable salt thereof.

The invention further relates to a compound selected from the group consisting of:

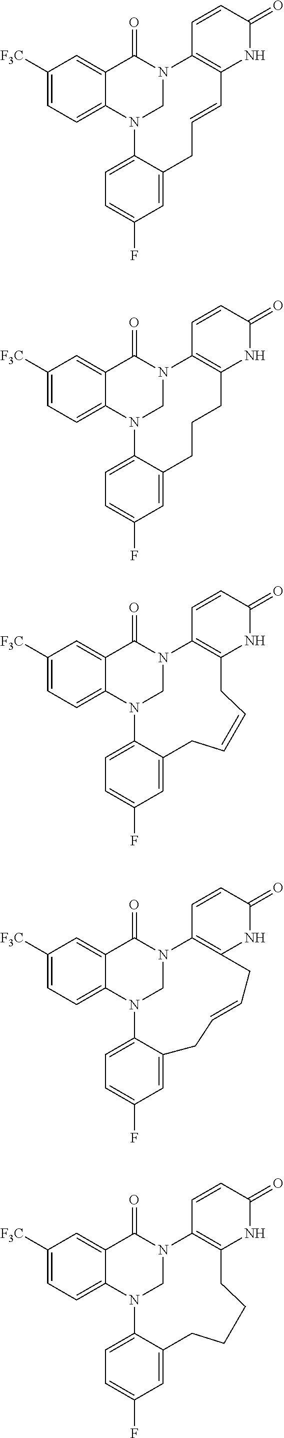 [SMILES: O=C1c2cc(C(F)(F)F)ccc2N2CN1c1ccc(=O)[nH]c1/C=C/Cc1cc(F)ccc12 ; O=C1c2cc(C(F)(F)F)ccc2N2CN1c1ccc(=O)[nH]c1CCCc1cc(F)ccc12 ; O=C1c2cc(C(F)(F)F)ccc2N2CN1c1ccc(=O)[nH]c1C/C=C\Cc1cc(F)ccc12 ; O=C1c2cc(C(F)(F)F)ccc2N2CN1c1ccc(=O)[nH]c1C/C=C/Cc1cc(F)ccc12 ; O=C1c2cc(C(F)(F)F)ccc2N2CN1c1ccc(=O)[nH]c1CCCCc1cc(F)ccc12]

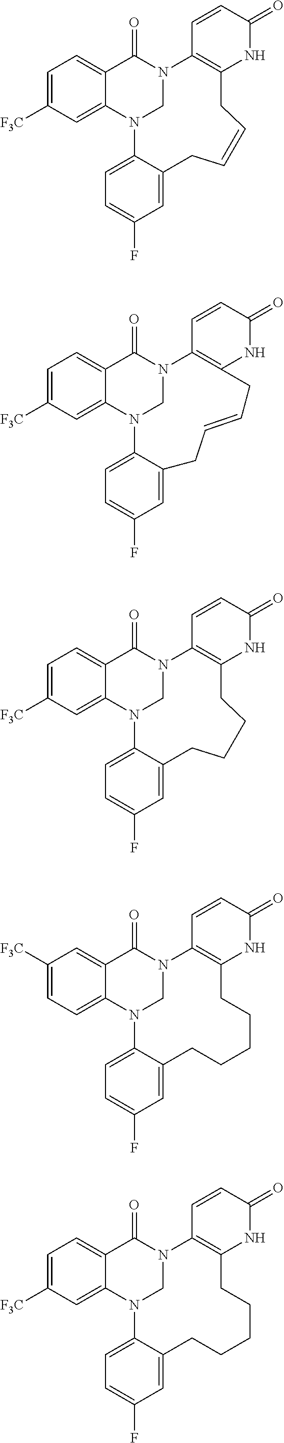 [SMILES: O=C1c2ccc(C(F)(F)F)cc2N2CN1c1ccc(=O)[nH]c1C/C=C\Cc1cc(F)ccc12 ; O=C1c2ccc(C(F)(F)F)cc2N2CN1c1ccc(=O)[nH]c1C/C=C/Cc1cc(F)ccc12 ; O=C1c2ccc(C(F)(F)F)cc2N2CN1c1ccc(=O)[nH]c1CCCCc1cc(F)ccc12 ; O=C1c2cc(C(F)(F)F)ccc2N2CN1c1ccc(=O)[nH]c1CCCCCc1cc(F)ccc12 ; O=C1c2ccc(C(F)(F)F)cc2N2CN1c1ccc(=O)[nH]c1CCCCCc1cc(F)ccc12]

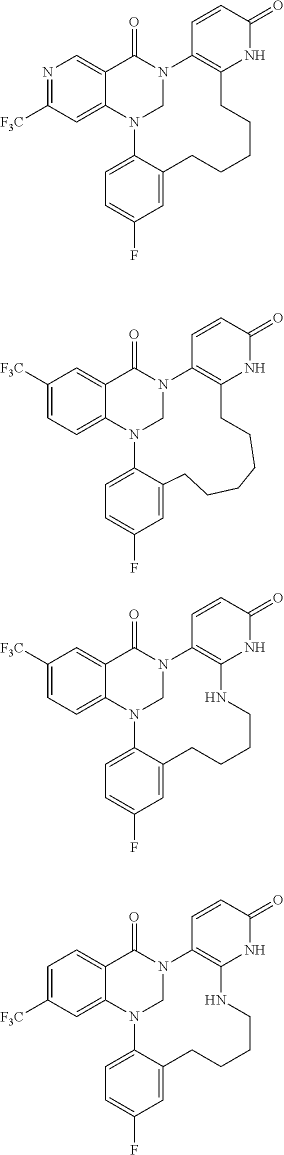

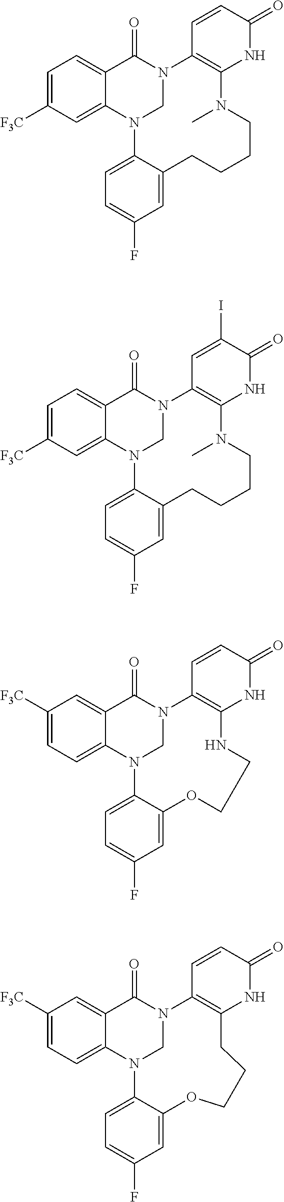 [SMILES: CN1CCCCc2cc(F)ccc2N2CN(C(=O)c3ccc(C(F)(F)F)cc32)c2ccc(=O)[nH]c21 ; CN1CCCCc2cc(F)ccc2N2CN(C(=O)c3ccc(C(F)(F)F)cc32)c2cc(I)c(=O)[nH]c21 ; O=C1c2cc(C(F)(F)F)ccc2N2CN1c1ccc(=O)[nH]c1NCCOc1cc(F)ccc12 ; O=C1c2cc(C(F)(F)F)ccc2N2CN1c1ccc(=O)[nH]c1CCCOc1cc(F)ccc12]

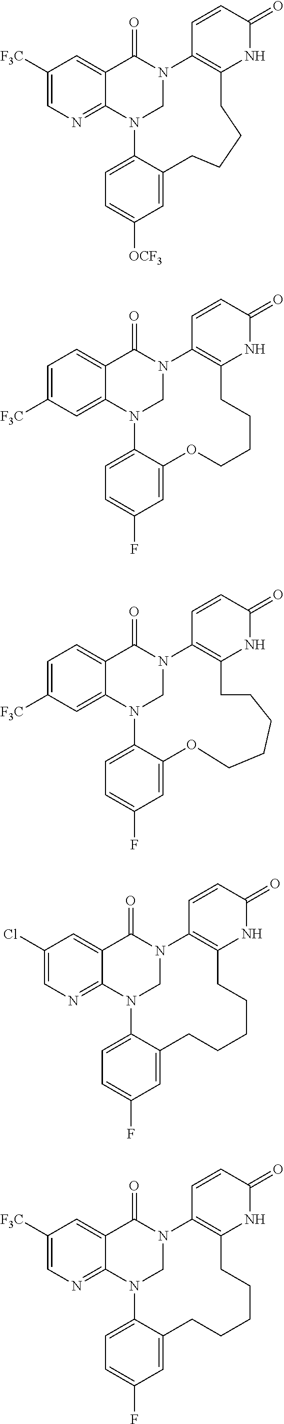 [SMILES: O=C1c2cc(C(F)(F)F)cnc2N2CN1c1ccc(=O)[nH]c1CCCCc1cc(OC(F)(F)F)ccc12 ; O=C1c2ccc(C(F)(F)F)cc2N2CN1c1ccc(=O)[nH]c1CCCCOc1cc(F)ccc12 ; O=C1c2ccc(C(F)(F)F)cc2N2CN1c1ccc(=O)[nH]c1CCCCCOc1cc(F)ccc12 ; O=C1c2cc(Cl)cnc2N2CN1c1ccc(=O)[nH]c1CCCCCc1cc(F)ccc12 ; O=C1c2cc(C(F)(F)F)cnc2N2CN1c1ccc(=O)[nH]c1CCCCCc1cc(F)ccc12]

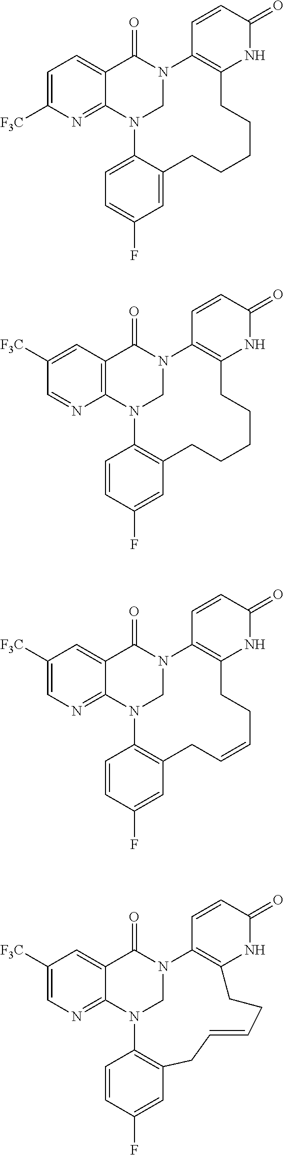 [SMILES: O=C1c2ccc(C(F)(F)F)nc2N2CN1c1ccc(=O)[nH]c1CCCCCc1cc(F)ccc12 ; O=C1c2cc(C(F)(F)F)cnc2N2CN1c1ccc(=O)[nH]c1CCCCCc1cc(F)ccc12 ; O=C1c2cc(C(F)(F)F)cnc2N2CN1c1ccc(=O)[nH]c1CC/C=C\Cc1cc(F)ccc12 ; O=C1c2cc(C(F)(F)F)cnc2N2CN1c1ccc(=O)[nH]c1CC/C=C/Cc1cc(F)ccc12]

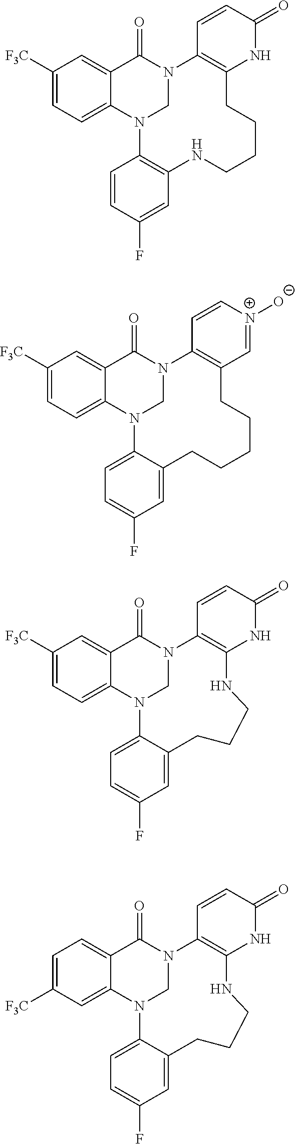 [SMILES: O=C1c2cc(C(F)(F)F)ccc2N2CN1c1ccc(=O)[nH]c1CCCCNc1cc(F)ccc12 ; O=C1c2cc(C(F)(F)F)ccc2N2CN1c1cc[n+]([O-])cc1CCCCCc1cc(F)ccc12 ; O=C1c2cc(C(F)(F)F)ccc2N2CN1c1ccc(=O)[nH]c1NCCCc1cc(F)ccc12 ; O=C1c2ccc(C(F)(F)F)cc2N2CN1c1ccc(=O)[nH]c1NCCCc1cc(F)ccc12]

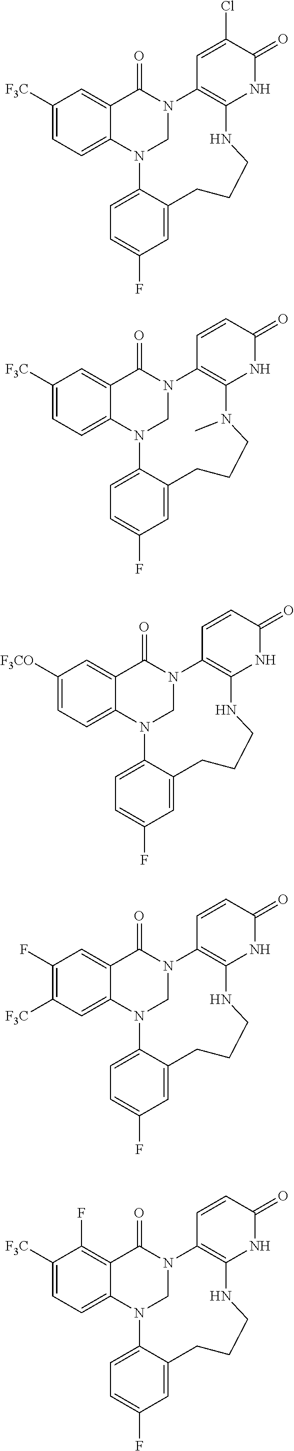

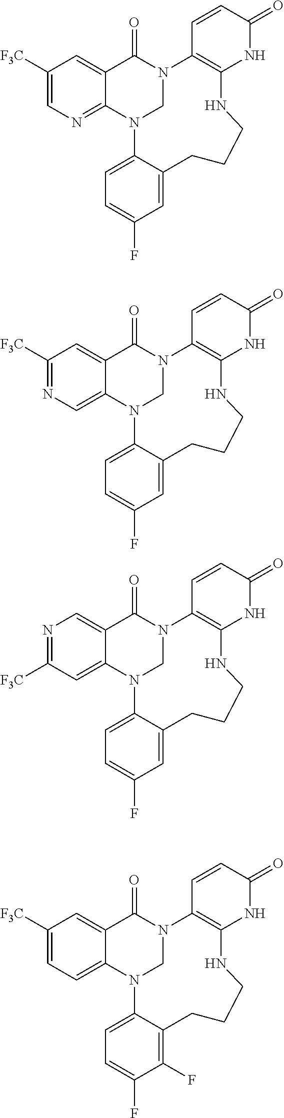

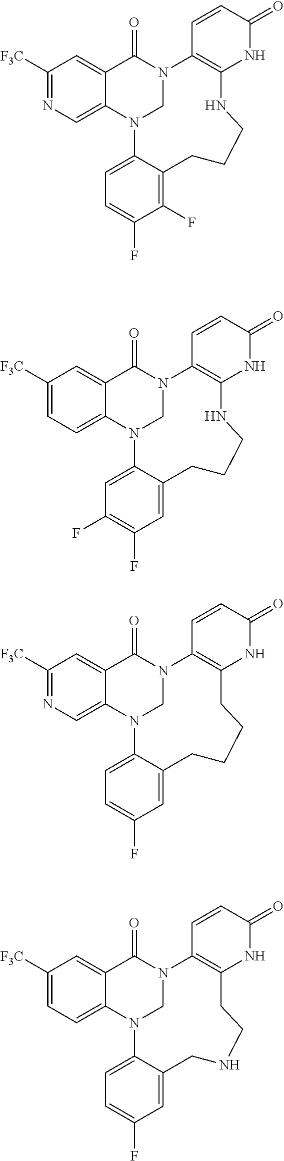 [SMILES: O=C1c2cc(C(F)(F)F)ncc2N2CN1c1ccc(=O)[nH]c1NCCCc1c2ccc(F)c1F ; O=C1c2cc(C(F)(F)F)ccc2N2CN1c1ccc(=O)[nH]c1NCCCc1cc(F)c(F)cc12 ; O=C1c2cc(C(F)(F)F)ncc2N2CN1c1ccc(=O)[nH]c1CCCCc1cc(F)ccc12 ; O=C1c2cc(C(F)(F)F)ccc2N2CN1c1ccc(=O)[nH]c1CCNCc1cc(F)ccc12]

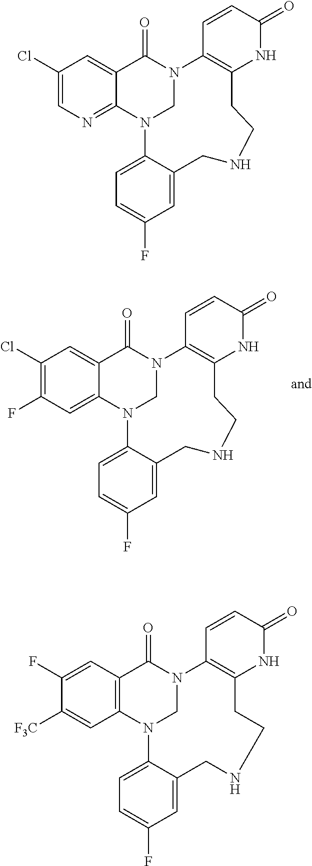 [SMILES: O=C1c2cc(Cl)cnc2N2CN1c1ccc(=O)[nH]c1CCNCc1cc(F)ccc12 ; O=C1c2cc(Cl)c(F)cc2N2CN1c1ccc(=O)[nH]c1CCNCc1cc(F)ccc12 ; O=C1c2cc(F)c(C(F)(F)F)cc2N2CN1c1ccc(=O)[nH]c1CCNCc1cc(F)ccc12]

or a tautomer thereof, or a pharmaceutically acceptable salt thereof.

In an embodiment, provided is a compound selected from the group consisting of:

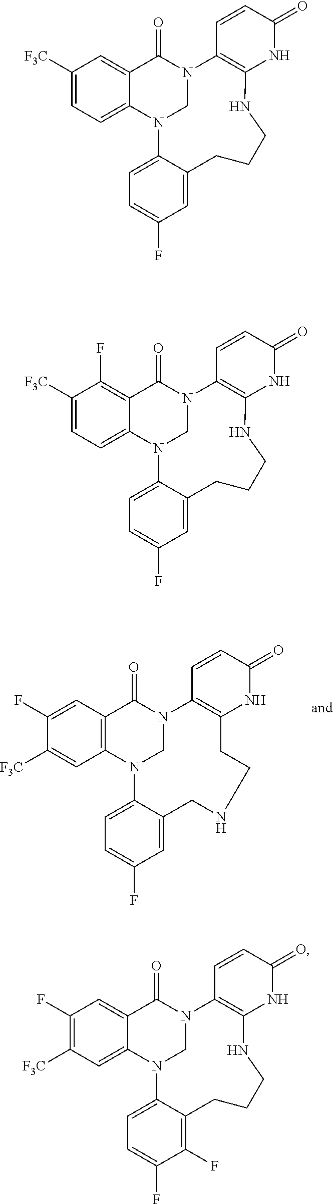 [SMILES: O=C1c2cc(C(F)(F)F)ccc2N2CN1c1ccc(=O)[nH]c1NCCCc1cc(F)ccc12 ; O=C1c2c(ccc(C(F)(F)F)c2F)N2CN1c1ccc(=O)[nH]c1NCCCc1cc(F)ccc12 ; O=C1c2cc(F)c(C(F)(F)F)cc2N2CN1c1ccc(=O)[nH]c1CCNCc1cc(F)ccc12 ; O=C1c2cc(F)c(C(F)(F)F)cc2N2CN1c1ccc(=O)[nH]c1NCCCc1c2ccc(F)c1F]

or a tautomer thereof, or a pharmaceutically acceptable salt thereof. In another embodiment, provided is a compound selected from the group consisting of:

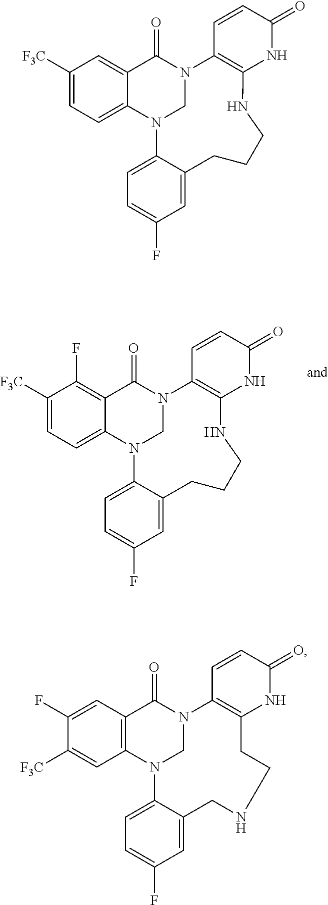 [SMILES: O=C1c2cc(C(F)(F)F)ccc2N2CN1c1ccc(=O)[nH]c1NCCCc1cc(F)ccc12 ; O=C1c2c(ccc(C(F)(F)F)c2F)N2CN1c1ccc(=O)[nH]c1NCCCc1cc(F)ccc12 ; O=C1c2cc(F)c(C(F)(F)F)cc2N2CN1c1ccc(=O)[nH]c1CCNCc1cc(F)ccc12]

or a tautomer thereof, or a pharmaceutically acceptable salt thereof. In another embodiment, provided is a compound which is

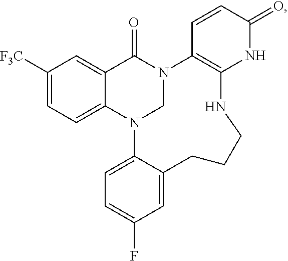 [SMILES: O=C1c2cc(C(F)(F)F)ccc2N2CN1c1ccc(=O)[nH]c1NCCCc1cc(F)ccc12]

or a tautomer thereof, or a pharmaceutically acceptable salt thereof. In another embodiment, provided is a compound which is:

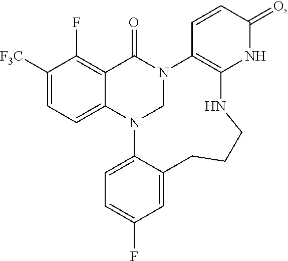 [SMILES: O=C1c2c(ccc(C(F)(F)F)c2F)N2CN1c1ccc(=O)[nH]c1NCCCc1cc(F)ccc12]

or a tautomer thereof, or a pharmaceutically acceptable salt thereof. In another embodiment, provided is a compound which is:

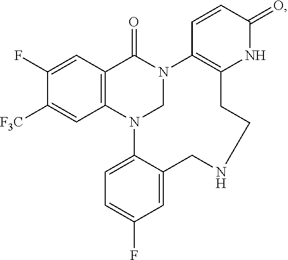 [SMILES: O=C1c2cc(F)c(C(F)(F)F)cc2N2CN1c1ccc(=O)[nH]c1CCNCc1cc(F)ccc12]

or a tautomer thereof, or a pharmaceutically acceptable salt thereof. In a further embodiment, provided is a compound which is:

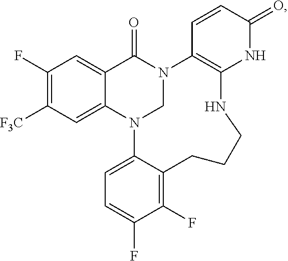 [SMILES: O=C1c2cc(F)c(C(F)(F)F)cc2N2CN1c1ccc(=O)[nH]c1NCCCc1c2ccc(F)c1F]

or a tautomer thereof, or a pharmaceutically acceptable salt thereof.

It is to be understood that the references herein to a compound of formula (I-a), (I), (II), (III), (IV), or (V) and/or corresponding tautomer forms thereof or a salt thereof includes a compound of formula (I-a), (I), (II), (III), (IV), or (V) and/or corresponding tautomer forms thereof as a free base or acid, or as a salt thereof, for example as a pharmaceutically acceptable salt thereof. Thus, in one embodiment, the invention is directed to a compound of formula (I-a), (I), (II), (III), (IV), or (V) and/or corresponding tautomer forms thereof. In another embodiment, the invention is directed to a salt of a compound of formula (I-a), (I), (II), (III), (IV), or (V) and/or corresponding tautomer forms thereof. In a further embodiment, the invention is directed to a pharmaceutically acceptable salt of a compound of formula (I-a), (I), (II), (III), (IV), or (V) and/or corresponding tautomer forms thereof. In another embodiment, the invention is directed to a compound of formula (I-a), (I), (II), (III), (IV), or (V) and/or corresponding tautomer forms thereof, or a salt thereof. In another embodiment, the invention is directed to a compound of formula (I-a), (I), (II), (III), (IV), or (V) and/or corresponding tautomer forms thereof, or a pharmaceutically acceptable salt thereof.

Because of its potential use in medicine, it will be appreciated that a salt of a compound of formula (I-a), (I), (II), (III), (IV), or (V) and/or corresponding tautomer forms thereof is preferably pharmaceutically acceptable.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. Furthermore, pharmaceutically acceptable salts of a compound of formulas (I)—(V) and/or corresponding tautomer forms thereof may be prepared during further processing of the free acid or base form, for example in situ during manufacture into a pharmaceutical formulation.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011.

Non-pharmaceutically acceptable salts may be used, for example as intermediates in the preparation of a compound of formula (I-a), (I), (II), (III), (IV), or (V) and/or corresponding tautomer forms thereof or a pharmaceutically acceptable salt thereof.

Suitable pharmaceutically acceptable salts can include acid or base addition salts. Such base addition salts can be formed by reaction of a compound of formula (I-a), (I), (II), (III), (IV), or (V) and/or corresponding tautomer forms thereof (which, for example, contains a carboxylic acid or other acidic functional group) with the appropriate base, optionally in a suitable solvent such as an organic solvent, to give the salt which can be isolated by a variety of methods, including crystallisation and filtration. Such acid addition salts can be formed by reaction of a compound of formula (I-a), (I), (II), (III), (IV), or (V) and/or corresponding tautomer forms thereof (which, for example contains a basic amine or other basic functional group) with the appropriate acid, optionally in a suitable solvent such as an organic solvent, to give the salt which can be isolated by a variety of methods, including crystallization and filtration.

Salts may be prepared in situ during the final isolation and purification of a compound of formula (I-a), (I), (II), (III), (IV), or (V) and/or corresponding tautomer forms thereof. If a basic compound of formula (I-a), (I), (II), (III), (IV), or (V) and/or corresponding tautomer forms thereof is isolated as a salt, the corresponding free base form of that compound may be prepared by any suitable method known to the art, including treatment of the salt with an inorganic or organic base. Similarly, if a compound of formula (I-a), (I), (II), (III), (IV), or (V) and/or corresponding tautomer forms thereof containing a carboxylic acid or other acidic functional group is isolated as a salt, the corresponding free acid form of that compound may be prepared by any suitable method known to the art, including treatment of the salt with an inorganic or organic acid.

It will be understood that if a compound of formula (I-a), (I), (II), (III), (IV), or (V) and/or corresponding tautomer forms thereof contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of formula (I-a), (I), (II), (III), (IV), or (V) and/or corresponding tautomer forms thereof are included within the scope of the invention, including sub-stoichiometric salts, for example where a counterion contains more than one acidic proton.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

It will be appreciated that many organic compounds can form complexes with solvents in which they are reacted or from which they are precipitated or crystallized. These complexes are known as “solvates.” For example, a complex with water is known as a “hydrate.” Solvents with high boiling points and/or solvents with a high propensity to form hydrogen bonds such as water, ethanol, iso-propyl alcohol, and N-methyl pyrrolidinone may be used to form solvates. Methods for the identification of solvates include, but are not limited to, NMR and microanalysis. Compounds of formula (I-a), (I), (II), (III), (IV), or (V) and/or corresponding tautomer forms thereof or salts thereof, may exist in solvated and unsolvated form.

The compounds of the invention may be in crystalline or amorphous form. The most thermodynamically stable crystalline form of a compound of the invention is of particular interest.

Crystalline forms of compounds of the invention may be characterized and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

Compounds of formula (I-a), (I), (II), (III), (IV), or (V) and/or corresponding tautomer forms thereof and pharmaceutically acceptable salts thereof may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of formula (I-a), (I), (II), (III), (IV), or (V) or in any chemical structure illustrated herein, is not specified the structure is intended to encompass all individual stereoisomers and all mixtures thereof. Thus, compounds of formula (I-a), (I), (II), (III), (IV), or (V) and/or corresponding tautomer forms thereof and pharmaceutically acceptable salts thereof containing one or more chiral centers may be used as racemic mixtures, enantiomerically enriched mixtures, or as enantiomerically pure individual stereoisomers.

Individual stereoisomers of a compound of formula (I-a), (I), (II), (III), (IV), or (V) and/or corresponding tautomer forms thereof or a pharmaceutically acceptable salt thereof, which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The invention also includes all suitable isotopic variations of a compound of formula (I-a), (I), (II), (III), (IV), or (V) and/or corresponding tautomer forms thereof or a pharmaceutically acceptable salt thereof. An isotopic variation of a compound of formula (I-a), (I), (II), (III), (IV), or (V) and/or corresponding tautomer forms thereof or a pharmaceutically acceptable salt thereof, is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, fluorine and chlorine such as ²H, ³H, ¹³C, ¹⁴C, ⁵N, ¹⁷O, ¹⁸O ¹⁸F and ³⁶Cl, respectively. Certain isotopic variations of a compound of formula (I-a), (I), (II), (III), (IV), or (V) and/or corresponding tautomer forms thereof or a salt or solvate thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of a compound of formula (I-a), (I), (II), (III), (IV), or (V) and/or corresponding tautomer forms thereof or a pharmaceutically salt thereof, can generally be prepared by conventional procedures such as by the illustrative methods or by the preparations described in the Examples hereafter using appropriate isotopic variations of suitable reagents.

Moreover, compounds of the invention may exist as tautomers or in tautomeric forms. It is to be understood that any reference to a named compound or structurally depicted compound is intended to encompass all tautomers of such compound. It is conventionally understood in the chemical arts that tautomers are structural or constitutional isomers of chemical compounds that readily interconvert. This reaction commonly results in the relocation of a proton. A structural isomer, or constitutional isomer (per IUPAC), is a type of isomer in which molecules with the same molecular formula have different bonding patterns and atomic organization, as opposed to stereoisomers, in which molecular bonds are always in the same order and only spatial arrangement differs. The concept of tautomerizations is called tautomerism. The chemical reaction interconverting the two is called tautomerization. Care should be taken not to confuse tautomers with depictions of ‘contributing structures’ in chemical resonance. Tautomers are distinct chemical species and can be identified as such by their differing spectroscopic data, whereas resonance structures are merely convenient depictions and do not physically exist. For example, the 2-pyridone ring exhibits tautomerism, wherein the proton attached to the nitrogen can move to the oxygen to give the tautomeric form 2-hydroxypyridine:

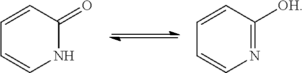 [SMILES: O=c1cccc[nH]1 ; Oc1ccccn1]

### Pharmaceutical Compositions

In another aspect, the invention relates to a pharmaceutical composition comprising a compound of formula (I-a), (I), (II), (III), (IV), or (V), or a tautomer thereof, or a pharmaceutically acceptable salt thereof, according to any one of the embodiments disclosed herein, and a pharmaceutically acceptable excipient (also referred to as carriers and/or diluents in the pharmaceutical arts). The excipients are acceptable in the sense of being compatible with the other ingredients of the formulation and not deleterious to the recipient thereof (i.e., the patient).

A pharmaceutically acceptable excipient is non-toxic and should not interfere with the efficacy of the active ingredient. Suitable pharmaceutically acceptable excipients will vary depending upon the particular dosage form chosen, route of administration, etc. Suitable pharmaceutically acceptable excipients include the following types of excipients: diluents, carriers, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. Examples of pharmaceutically acceptable excipients are described, e.g., in Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

Pharmaceutical compositions may be adapted for administration by any appropriate or suitable route, for example by systemic administration (e.g., oral administration, parenteral administration, transdermal administration, rectal administration, inhalation), topical administration, etc. Parenteral administration is typically by injection or infusion and includes intravenous, intramuscular, and subcutaneous injection or infusion. Inhalation refers to administration into the patient's lungs whether inhaled through the mouth or through the nasal passages. Typically, administration is via the oral route or parenteral route.

Pharmaceutical compositions adapted for oral administration may be presented as solid dosage forms such as tablets, capsules, caplets, troches, pills; powders; or liquid dosage forms such as solutions, suspensions, syrups, elixirs, or emulsion, etc. Pharmaceutical compositions adapted for parenteral administration may be presented as solutions, suspensions, and powders for reconstitution.

In general, pharmaceutical compositions of the invention are prepared using conventional materials and techniques, such as mixing, blending and the like. Some of the methods commonly used in the art are described in Remington's Pharmaceutical Sciences (Mack Publishing Company).

Solid oral dosage forms, such as tablets and capsules can be prepared by mixing a compound of the invention with excipients such as diluents and fillers (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like. Pharmaceutical compositions adapted for parenteral administration can be an injection solution prepared from powders, granules or tablets by mixing with a carrier, such as distilled water, saline and the like, and base and the like may be used for pH adjustment.

The invention also provides a pharmaceutical composition comprising from 0.5 to 1,000 mg of a compound of the invention and from 0.5 to 1,000 mg of a pharmaceutically acceptable excipient.

Compounds and pharmaceutical compositions of the invention as defined herein may be administered once or according to a dosing regimen, where a number of doses are administered at varying intervals of time for a given period of time. For example, doses may be administered one, two, three, or four times per day. Doses may be administered until the desired therapeutic effect is achieved or indefinitely to maintain the desired therapeutic effect. Doses of compounds of the invention may in the range of 0.001 mg/kg to 100 mg/kg, such as 0.001 mg/kg to 50 mg/kg. Preferably, the selected dose is administered orally or parenterally.

In accordance with another aspect of the invention there is provided a process for the preparation of a pharmaceutical composition comprising mixing (or admixing) a compound of formula (I-a), (I), (II), (III), (IV), or (V), or a tautomer thereof or salt thereof (e.g., pharmaceutically acceptable salt thereof) with at least one pharmaceutically acceptable excipient.

### Synthetic Schemes and General Preparation

The invention also relates to processes for preparing compounds of the invention disclosed herein. The compounds of the invention may be made by any number of processes using conventional organic syntheses as described in the Schemes below and more specifically illustrated by the exemplary compounds which follow in the Examples section herein, or by drawing on the knowledge of a skilled organic chemist. Suitable synthetic routes are depicted below in the following general reaction schemes. The synthesis procedures provided in the following Schemes are applicable for producing compounds of the invention disclosed herein, having a variety of different functional groups as defined employing appropriate precursors.

Those skilled in the art will appreciate that in the preparation of compounds of the invention, it may be necessary and/or desirable to protect one or more sensitive groups in the molecule or the appropriate intermediate to prevent undesirable side reactions. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups for use according to the present invention are well-known to those skilled in the art and may be used in a conventional manner. See for example, “Protective Groups in Organic Synthesis” by T.W. Green and P.G.M Wets (Wiley & Sons, 1991) or “Protecting Groups” by P. J. Kocienski (Georg Thieme Verlag, 1994). Subsequent deprotection, where needed, affords compounds of the nature generally disclosed. In some instances, a substituent may be specifically selected to be reactive under the reaction conditions used. Under these circumstances, the reaction conditions convert the selected substituent into another substituent that is either useful as an intermediate compound or is a desired substituent in a target compound.

While the Schemes shown below are representative of methods for preparing compounds of the invention, they are only intended to be illustrative of processes that may be used to make the compounds of the invention. Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof. Compound names were generated using the software naming program ChemDraw 5 Ultra v12.0, available from Perkin Elmer, 940 Winter Street, Waltham, Massachusetts, 02451, USA.

Several methods for preparing the compounds of this invention are illustrated in the following Schemes and Examples. Starting materials are either commercially available or made by known procedures in the literature or as illustrated.

### General Synthetic Schemes

In the following generic schemes, including Generic Schemes 1-16, each R¹ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-; each R² is independently hydrogen, halo, —(C₁-C₆)alkyl, —(C₁-C₆)alkoxy, halo(C₁-C₆)alkyl-, or halo(C₁-C₆)alkoxy-; and

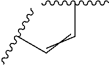 [SMILES: *C=C*]

represents an alkenylene present as a mixture of cis and trans isomers.

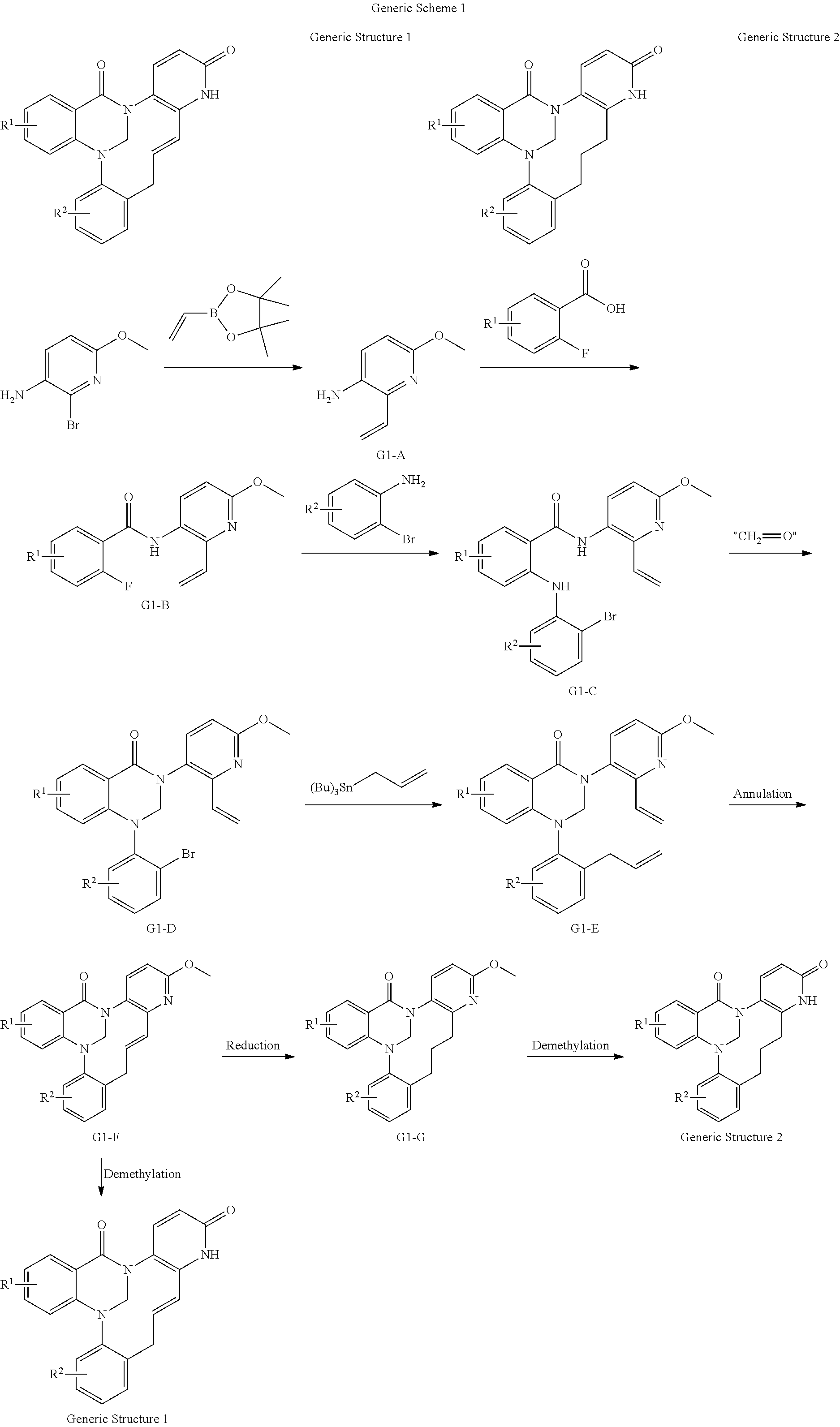

Compounds exemplified herein with Generic Structures 1 and 2 can be prepared by the general sequence outlined in Generic Scheme 1. Cross-coupling of a vinyl borate substrate, such as vinyl dioxaboralane, boronic acid, or potassium trifluoroborate, with 2-bromo-6-methoxypyridin-3-amine using a palladium catalyst, such as Pd(PPh₃)₄, an inorganic base, such as sodium carbonate in organic solvents, such as dioxane or toluene, at elevated temperatures, gives the intermediate G1-A. G1-A undergoes amide coupling with a R¹-substituted 2-fluorobenzoic acid in the presence of a coupling reagent, such as HATU, pyoxim or T3P, and organic base, such as DIEA, TEA or NMM, to produce G1-B. Nucleophilic displacement with a R²-substituted 2-bromoaniline using an inorganic base, such as cesium carbonate, at elevated temperatures, gives structure G1-C, which undergoes dihydopyrimidinone formation using diiodomethane or chloroiodomethane as a formaldehyde equivalent. In this variant of the cyclization reaction, a base, typically Cs₂CO₃ or NaH, could be used, in a suitable solvent, oftentimes acetonitrile or DMF, at elevated temperature to G1-D. A Stille cross-coupling using a catalyst, such as Pd(PPh₃)₄, at elevated temperature, results in the bisalkene G1-E which undergoes Grubbs-catalyzed annulation at elevated temperatures, to form the macrocycle G1-F. This compound can be converted to the Generic Structure 1 via treatment with an alkali metal, such as lithium chloride, in the presence of a strong organic acid, such as tosic acid, at elevated temperature, or hydrogenated, using a palladium source, such as Pd(OH)₂, and hydrogen atmosphere in an alcoholic solvent, such as methanol or ethanol, prior to conversion to the pyridinone Generic Structure 2.

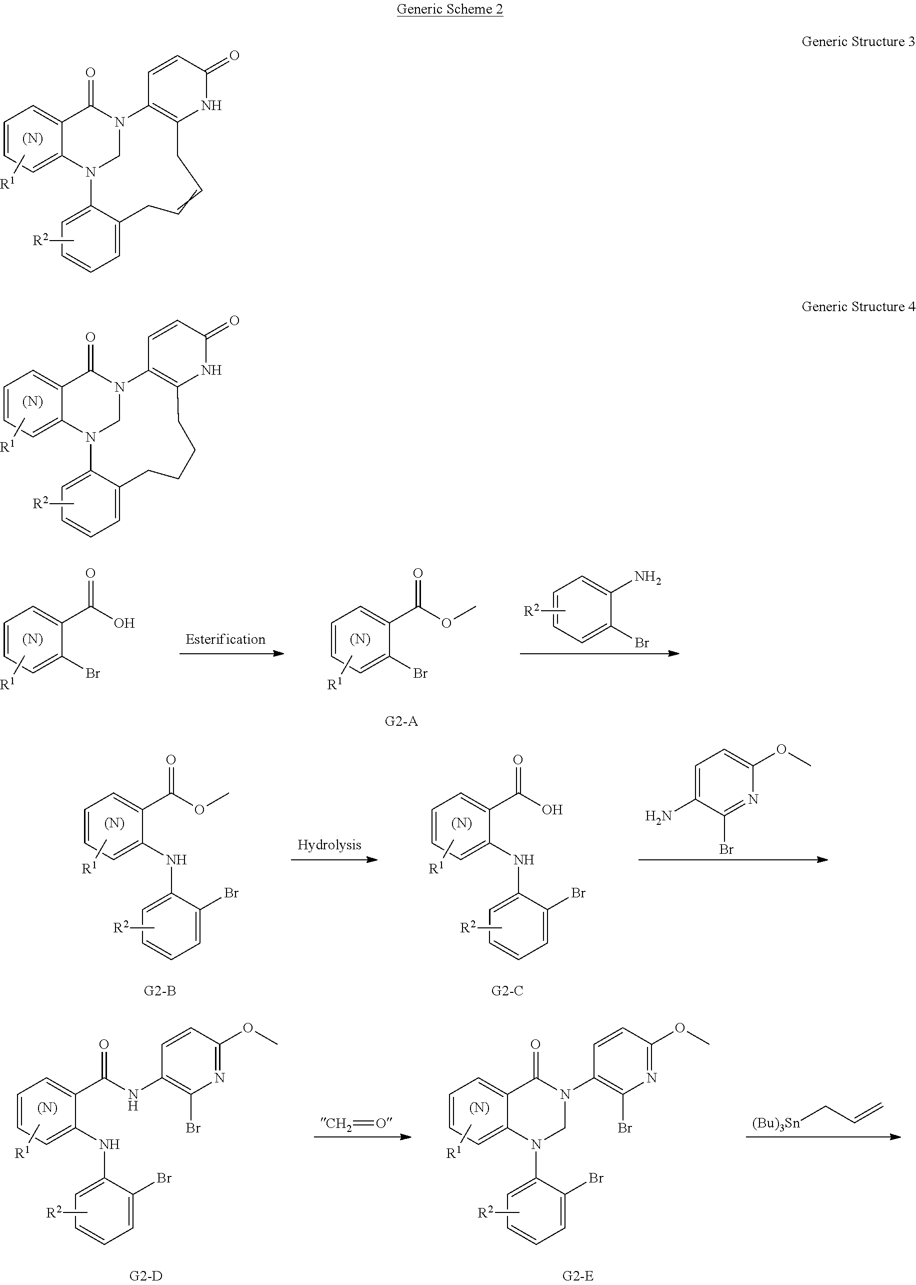

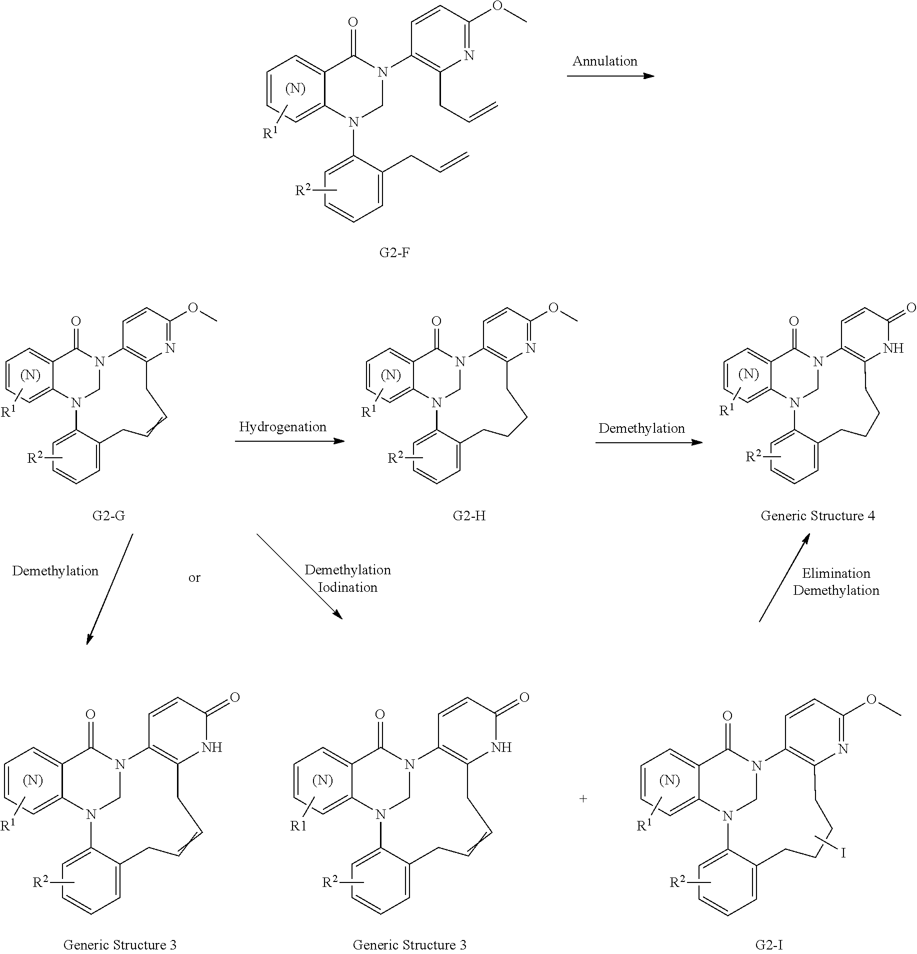

Compounds with Generic Structures 3 and 4 can be prepared by the general sequences outlined in Generic Scheme 2. An R¹-substituted o-bromoarylcarbonic acid can be esterified using strong organic acid, such as sulfuric acid, with a suitable alcohol, such as methanol, to afford G2-A. The ester can undergo a catalyzed-mediated buchwald coupling, in the presence of a catalyst, for example Pd₂(dba)₃ or Pd(OAc)₂, and a suitable ligand, for instance BINAP or Xantphos, and conducted at elevated temperature in the presence of an inorganic base, typically NaO^{f}Bu, Cs₂CO₃ or K₃PO₄, in an appropriate solvent, such as 1,4-dioxane, toluene or DMSO, with a R²-substituted bromoaniline, to yield intermediate G2-B, which is subsequently hydrolyzed under basic conditions. Saponification of the ester G2-B to the corresponding acid G2-C is typically achieved under standard basic conditions, using bases such as LiOH, KOH, or NaOH, in a suitable solvent or solvent system, for instance methanol/H₂O, ethanol/H₂O, THF/H₂O, or THF/MeOH/H₂O. G2-C is amide coupled to 2-bromo-6-methoxypyridin-3-amine to afford the dibromo structure G2-D. For example, one might employ standard coupling reagents, like EDC/HOBT, HATU, HBTU or T3P, in the presence of an amine base, like triethylamine, or Huinig's base (diisopropylethylamine), in a suitable solvent, typically DMF, DMA or acetonitrile. Dihydropyrimidinone formation using the aforementioned conditions in generic scheme 1, followed by a dual Stille cross-coupling results in the intermediate G2-F. In a typical Stille coupling, a palladium source, such as Pd(PPh₃)₄ (tetrakis(triphenylphosphine)palladium) and an organostannane, such as allyltributyltin, in a suitable solvent, such as DMF or toluene, at elevated temperature, are employed. A Grubbs-catalyzed annulation, using a Hoveyda-Grubbs catalyst, produces the macrocycle G2-G, which yields Generic Structure 3 upon demethylation. Preferred methods for achieving this transformation include utilizing a mixture of p-toluenesulfonic acid and LiCl in a solvent such as DMF at elevated temperature or TMS-iodide, in a neutral solvent like acetonitrile, at elevated temperature to afford intermediate G2-I. G2-I can be treated with a non-nucleophilic base, such as DBU, in a solvent such as DMF, then hydrogenated using the aforementioned conditions, to give Generic Structure 4. Alternately, G2-G is hydrogenated before demethylation using the aforementioned conditions to the pyridinone Generic Structure 4.

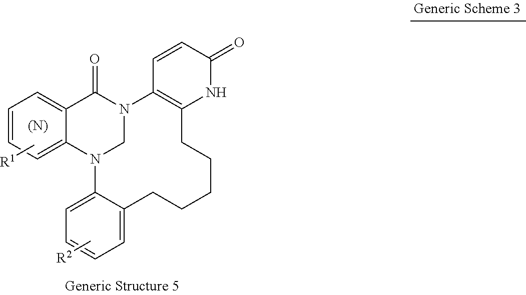

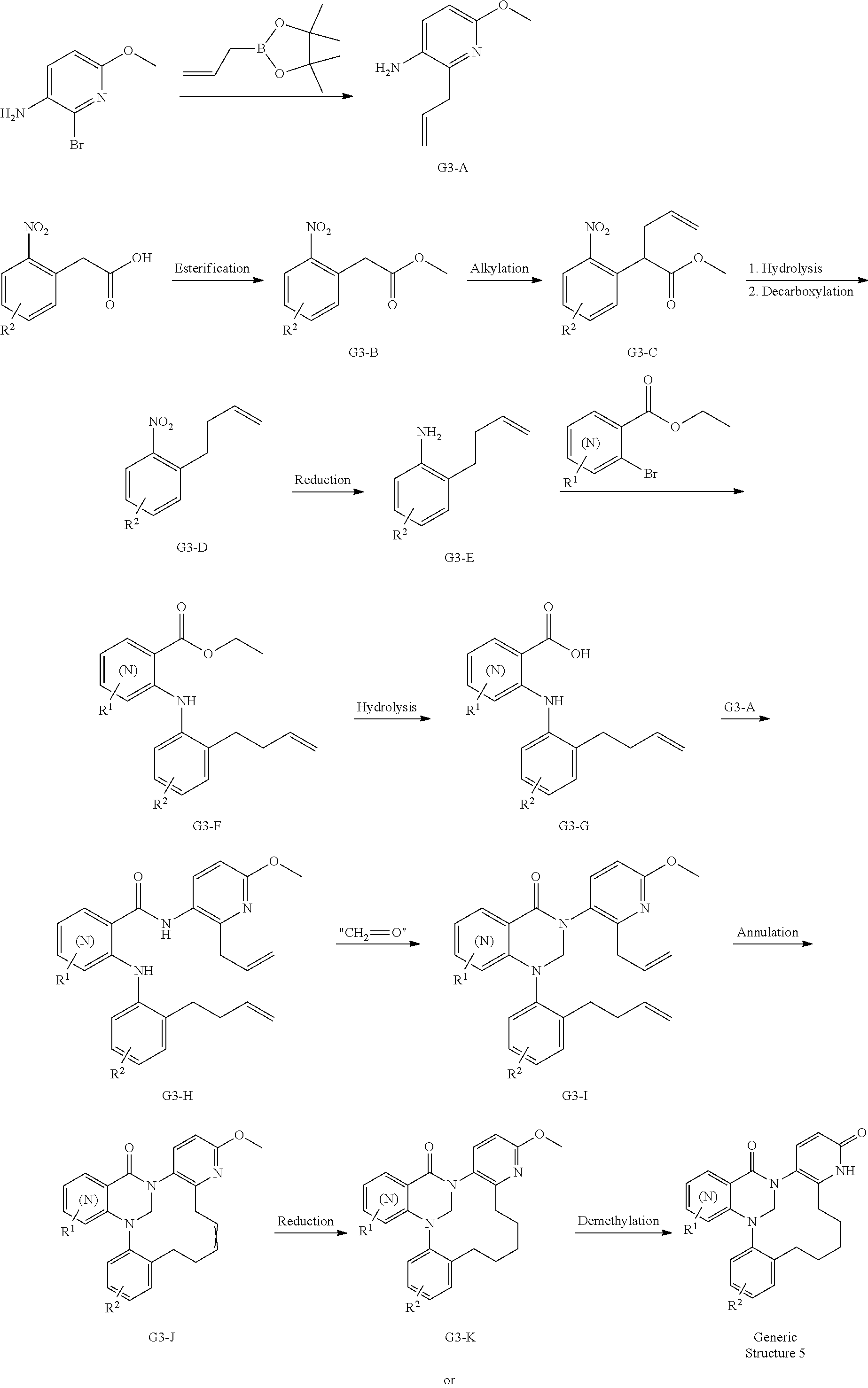

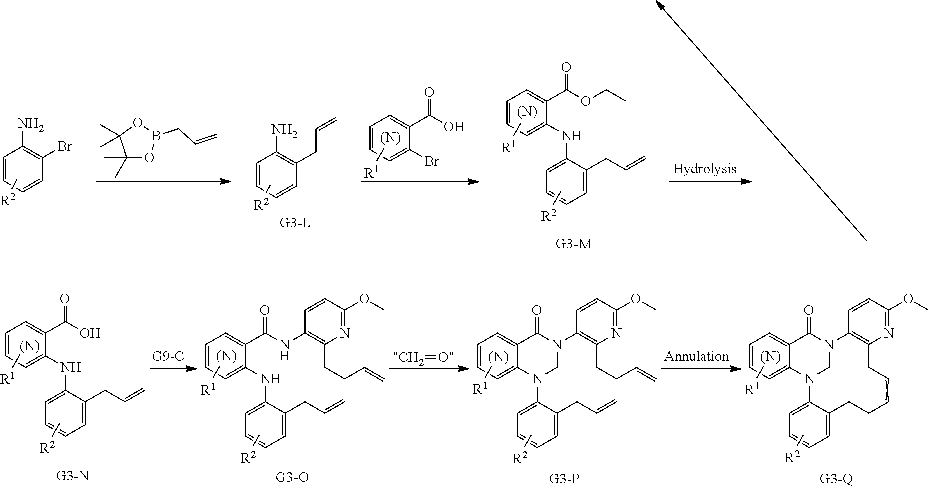

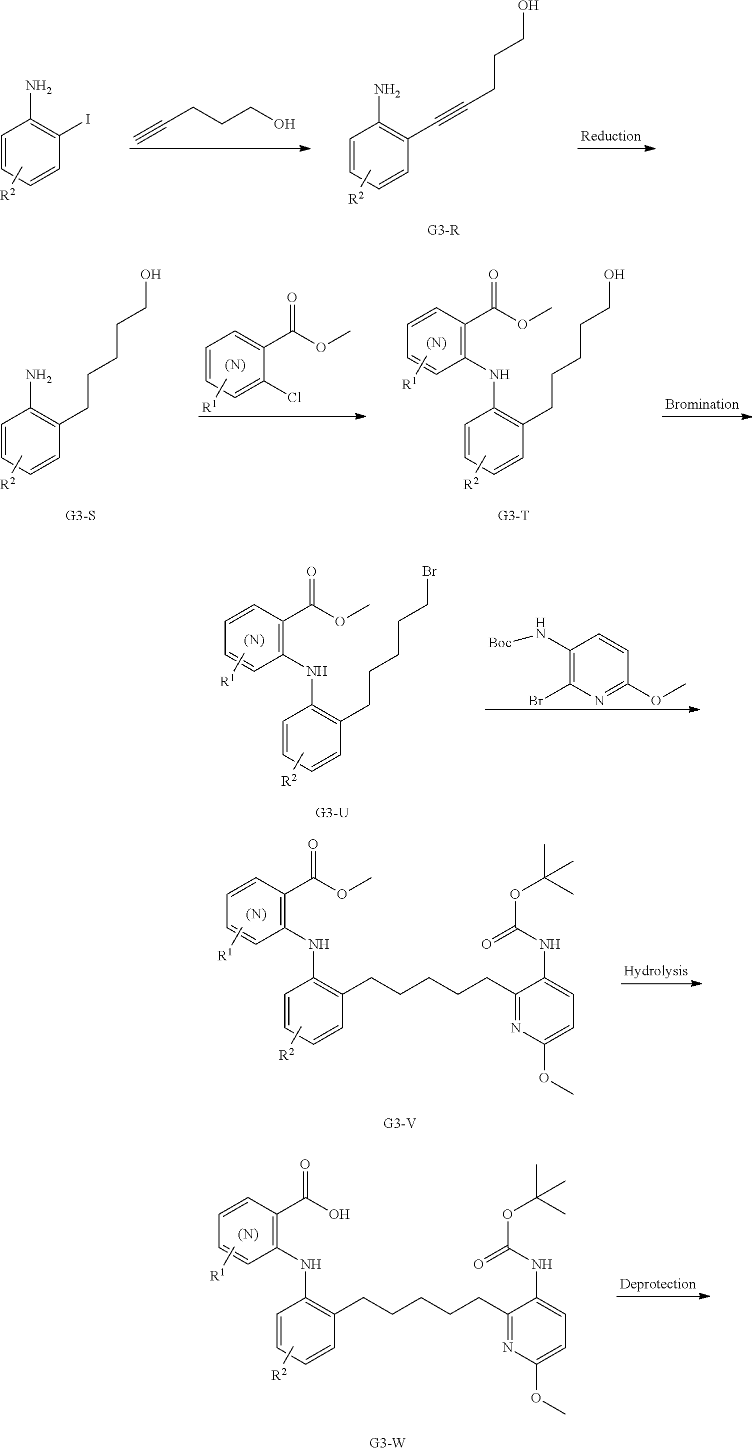

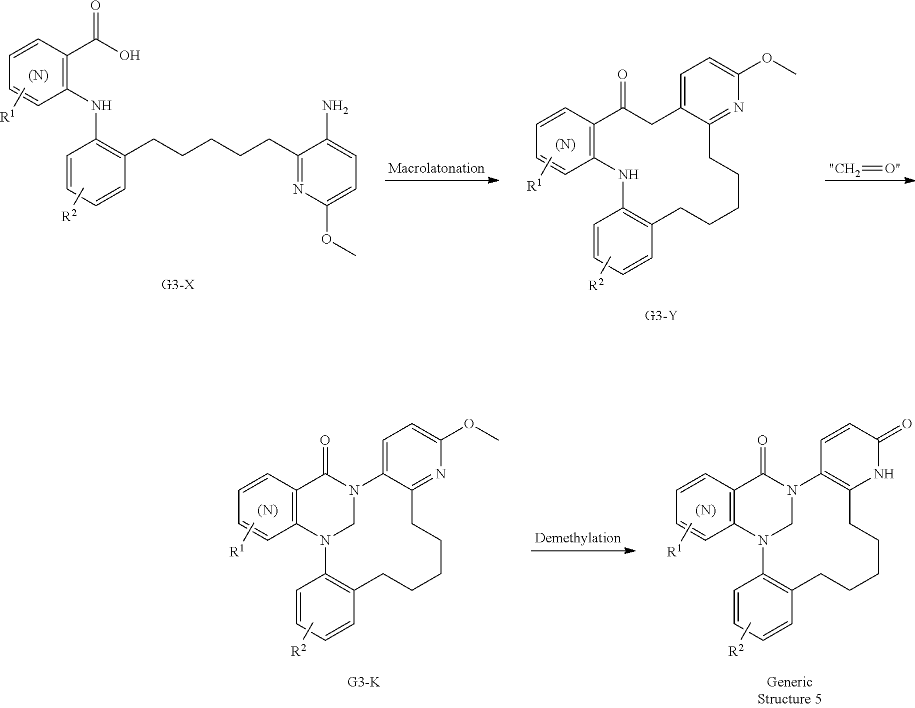

Compounds with Generic Structure 5 can be prepared by the general sequences outlined in Generic Scheme 3. Suzuki cross-coupling performed of an allyl borane, such as allyl dioxaboralane, boronic acid, or potassium trifluoroborates with 2-bromo-6-methoxypyridin-3-amine gives the intermediate G3-A. Such reactions typically use a palladium catalyst, such as Pd(PPh₃)₄, an inorganic base such as cesium fluoride in organic solvents, such as THF, dioxane, or toluene, at elevated temperatures. Concurrently, an R²-substituted o-nitrophenylacetic acids are esterified using a strong protic acid, such as HCl or H₂SO₄, in the appropriate alcoholic solvent, such as MeOH, and alkylated under mild basic conditions, using an inorganic base such as NaH, K₂CO₃ or Cs₂CO₃, in the presence of a crown ether and neutral solvent, such as MeCN, to give G3-C. Subsequent ester hydrolysis using an organic base such as sodium hydroxide in the appropriate solvent, such as dioxane, followed by decarboxylation of this intermediate (not shown), using an inorganic base, such as potassium carbonate at elevated temperature affords G3-D. Reduction of the nitro group using a metal source, such as zinc or iron, in alcohol solvent, such as ethanol, in the presence of an acid source, such as acetic acid, at elevated temperatures, produces G3-E. Buchwald cross coupling of aniline G3-E with a R¹-substituted bromoarylcarbonate followed by ester hydrolysis using the aforementioned conditions affords G3-G. Amide coupling with G3-A followed by dihyropyrimidinone ring formation using the previously outlined conditions produces G3-I, which undergoes Grubbs-catalyzed annulation to macrocycle G3-J. Using the aforementioned procedures, Generic Structure 5 is formed after catalytic reduction and demethylation to the pyridinone ring.

Alternatively, using previously cited procedures, R²-substituted o-bromoanilines can undergo a Suzuki cross-coupling to G3-L followed by Buchwald coupling with a R¹-substituted bromoarylcarbonate to give G3-M. Subsequent base-mediated hydrolysis to G3-N and amide formation with G9-C (see below) using previously described conditions produces the bisalkene G3-O. Dihydropyrimidinone formation followed by Grubbs-catalyzed annulation forms the macrocycle G3-Q. A Grubbs borohydride reduction of the tethered alkene yields the intermediate G3-K which is converted to Generic Structure 5 as described previously.

Another approach involves preforming a Sonogashira cross-coupling with a R²-substituted o-iodoaniline with 5-hydroxypentyne to G3-R. Typical conditions employ a palladium/ligand source, such as bis(triphenylphosphine)palladium(II) dichloride/triphenylphosphine ([Pd(PPh₃)₂Cl₂])/PPh₃) or Pd(PPh₃)₄, and a copper (I) halide salt co-catalyst, such as copper(I)I, in the presence of an organic base, such as TEA DEA, or DIEA, in the appropriate solvent, such as DMF and a terminal alkyne, at elevated temperatures. The aforementioned conditions for the catalytic hydrogenation (G3-S) and buchwald cross coupling to a R¹-substituted chloroarylcarbonate steps afford G3-T. Triphenylphosphine mediated conversion to the terminal bromide with tetramethylbromide followed by subsequent negishi coupling to BOC-ed 2-bromo-3-amino-6-methoxypyridine gives G3-V. Typical conditions employ a metal catalyst, such as zinc, a metal halide, such a nickel (II) chloride, in the presence of an inorganic salt, such as sodium iodide, and ligand, such as picolinimidamide, in the appropriate solvent, such as dimethylacetamide (DMA) at elevated temperatures. Ester hydrolysis (G3-W), amine deprotection (G3-X), using a strong acid such as TFA or HCl, and intramolecular amide coupling using aforementioned conditions, form the macrocycle G3-Y. Formation of the dihydropyrimidinone yields G3-K, which is demethylated to Generic Structure 5 using previously outlined conditions.

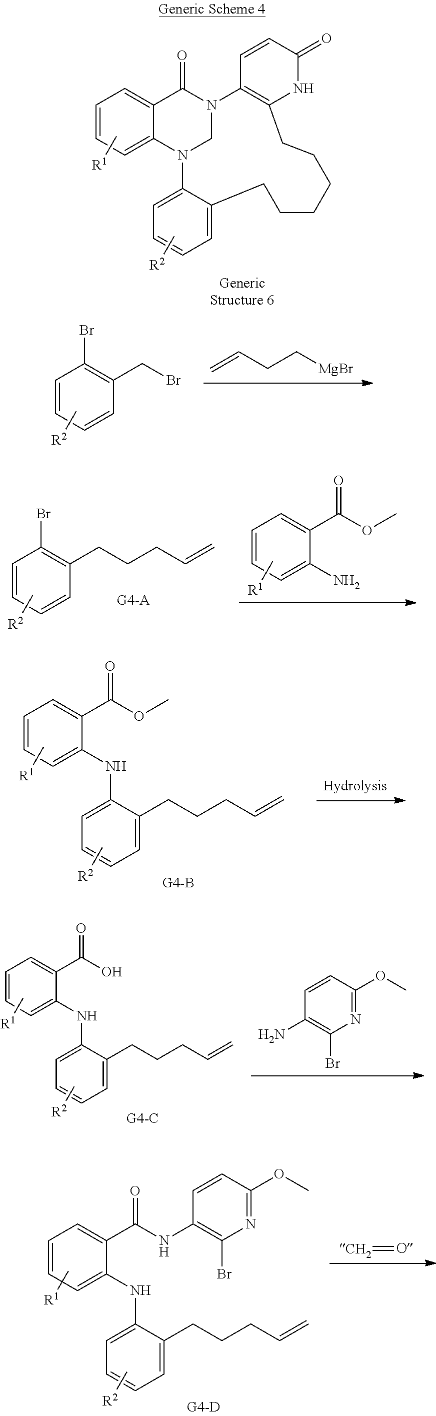

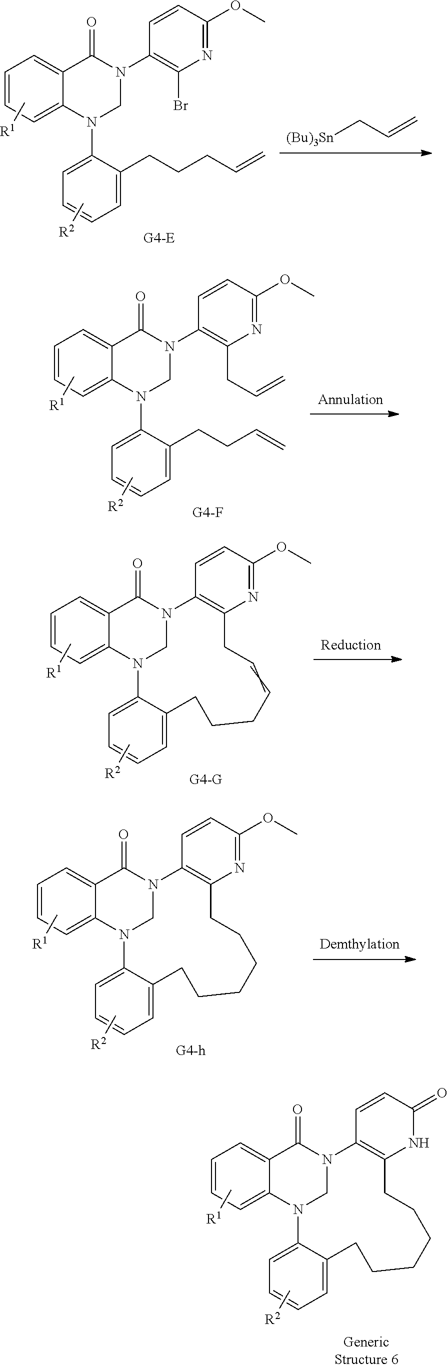

Compounds with Generic Structure 6 can be prepared by the general sequence outlined in Generic Scheme 4. Cross coupling of a benzyl halide, such as R¹-substituted o-bromobenzyl bromides, with the requisite grignard, using a copper source, such as CuI, and a ligand, such as 2-,2′-bipyridine, in the appropriate solvent, such as toluene, at reduced temperatures, forms G4-A. Aforementioned buchwald cross-coupling (G4-B), ester hydrolysis (G4-C), amide coupling (G4-D) and dihydropyrimidone formation affords intermediate G4-E. Stille cross-coupling (G4-F) followed by annulation using previously described methods yields G4-G. Generic Structure 6 is prepared utilizing conditions mentioned in above generic schemes for the catalytic hydrogenation and demethylation steps.

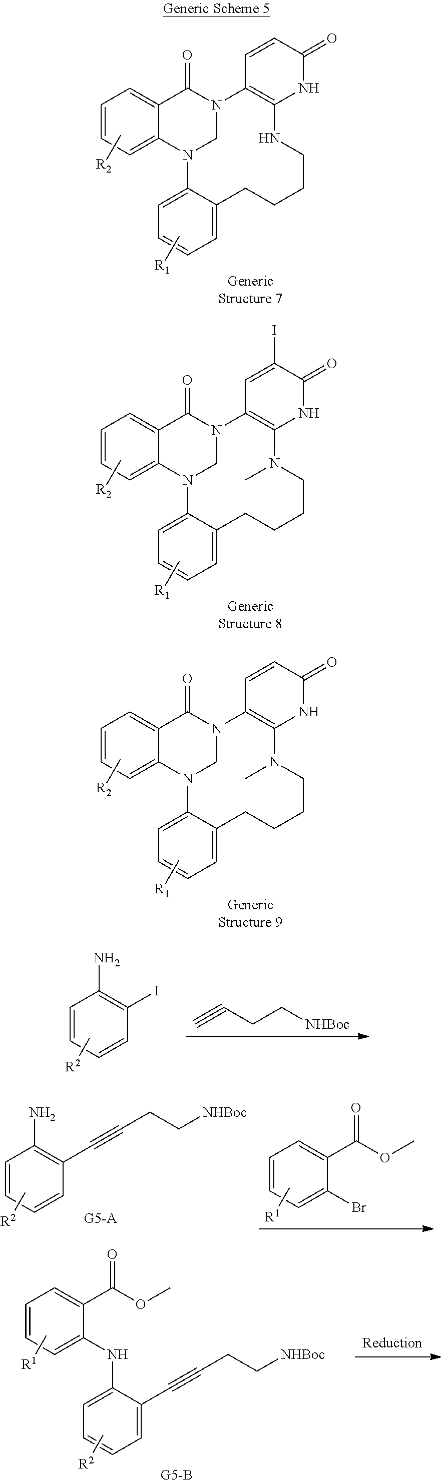

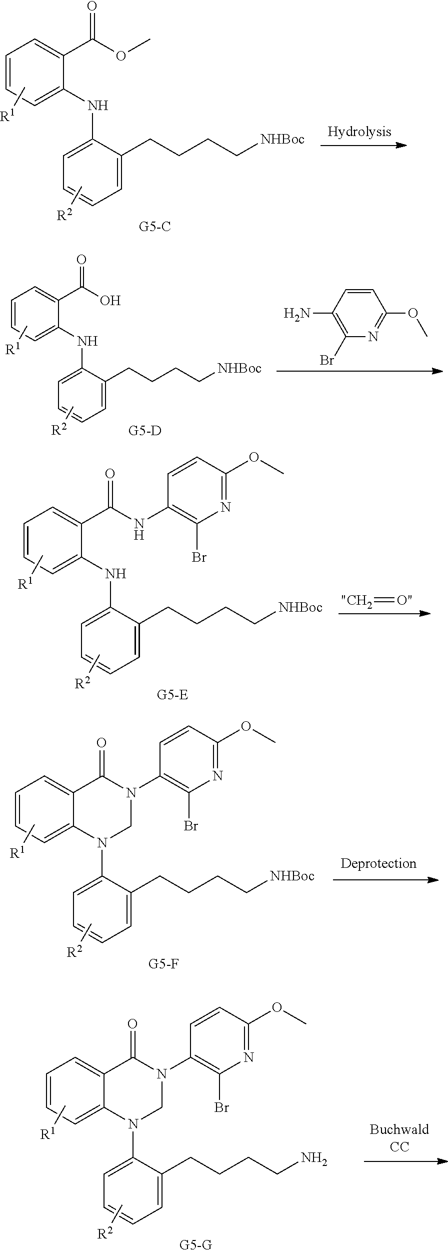

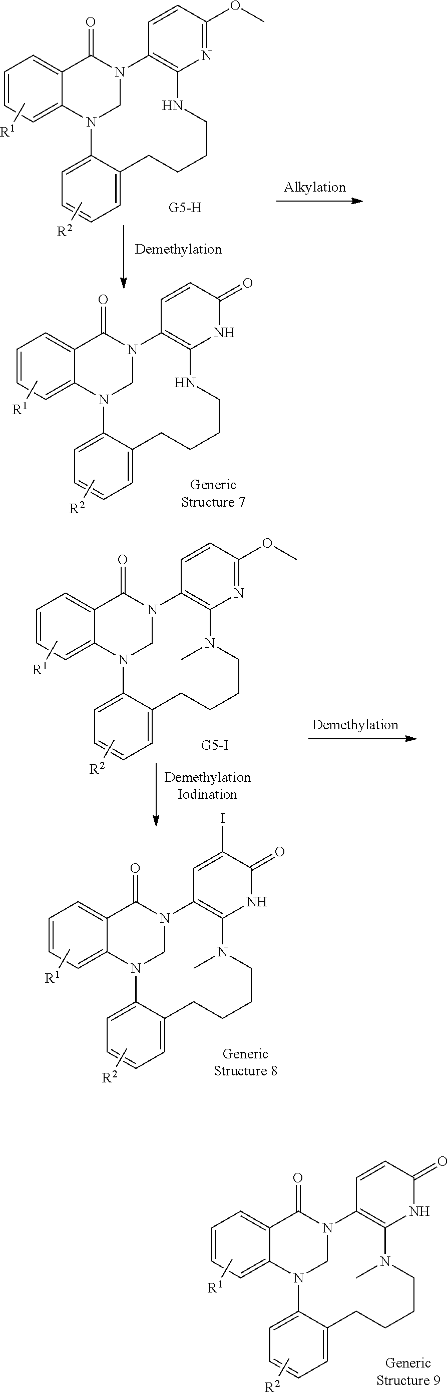

Compounds with Generic Structures 7, 8 and 9 can be prepared by the general sequence outlined in Generic Scheme 5. R²-substituted o-iodoanilines can undergo Sonogashira coupling to a terminal alkyne, such as BOC-ed 4-aminobutyne, to yield G5-A. Typical conditions employ a palladium/ligand source, such as bis(triphenylphosphine)palladium(II) dichloride/triphenylphosphine ([Pd(PPh₃)₂Cl₂])/PPh₃) or Pd(PPh₃)₄, and a copper (I) halide salt co-catalyst, such as copper(I)I, in the presence of an organic base, such as TEA, DIA, or DIEA, in the appropriate solvent, such as diethyl ether or acetonitrile. Subsequent buchwald coupling with R¹-substituted bromophenylesters using aforementioned conditions produce G5-B. Catalytic hydrogenation of the alkyne (G5-C), using a palladium source, such as Pd—C, in the appropriate solvent, such as EtOAc, EtOAc/EtOH, under hydrogen atmosphere, followed by ester hydrolysis using previously described conditions affords G5-D. Using aforementioned conditions for the amide coupling (G5-E) and dihydropyrimidone ring closure provides G5-F. Acid deprotection using a strong organic acid, such as TFA or HCl, in the appropriate solvent, such as dichloromethane or dioxane, affords G5-G, which undergoes an intramolecular ring closure to provide G5-H. Typical conditions employ a catalyst-ligand system, such as Pd₂(dba)₂/BINAP, using a base, such as NaOtBu and solvent such as dioxane or toluene, at elevated temperature to afford G5-H. This macrocycle can be converted to Generic Structure 7 using previously described methods, or N-alkylated with an alkylating agent, such as iodomethane, upon treatment with an inorganic base, such as sodium hydride, to produce G5-I. Using sodium iodide in the presence of trimethylsilyl-chloride, the latter intermediate yields Generic Structure 8. Alternatively, G5-I can be demethylated with HBr in acetic acid to form Generic Structure 9.

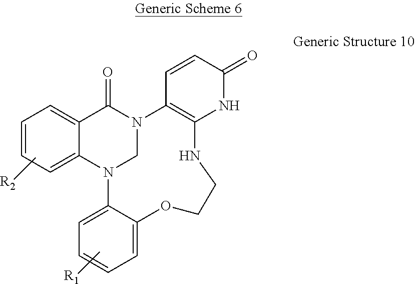

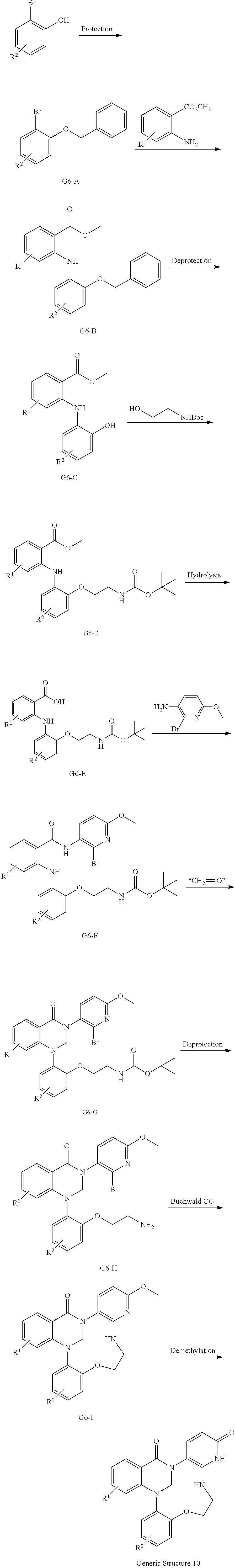

Compounds with Generic Structure 10 can be prepared by the general sequence outlined in Generic Scheme 6. R²-substituted o-bromophenols can be appropriately protected, such as employing a benzyl protecting group, installed using a base, such as potassium carbonate, a solvent, such as acetone, and benzyl bromide at elevated temperatures, and subsequently coupled with R¹-substituted o-aminobenzoates using previously described buchwald cross-coupling conditions to produce G6-B. Deprotection of the phenol by catalytic hydrogenation conditions, in which a palladium catalyst, such as Pd—C or Pd(OH)₂, and an appropriate solvent, such as ethanol, are used to afford G6-C. Mitsunobu coupling of the phenol with N—BOC-ed 2-aminoethanol, using an azodicarboxylate, such as DEAD, DIAD or the like, in the presence of triphenylphosphine, and the appropriate solvent, such as THE or toluene, intermediate G6-D is formed. Using the aforementioned methods for the ester hydrolysis (G6-E), amide coupling with 2-bromo-3-amino-6-methoxypyridine (G6-F), and dihydropyrimidinone ring formation yields G6-G. Subsequent deprotection of the amine (G6-H), intramolecular ring closure (G6-I), and demethylation using previously described steps yield Generic Structure 10.

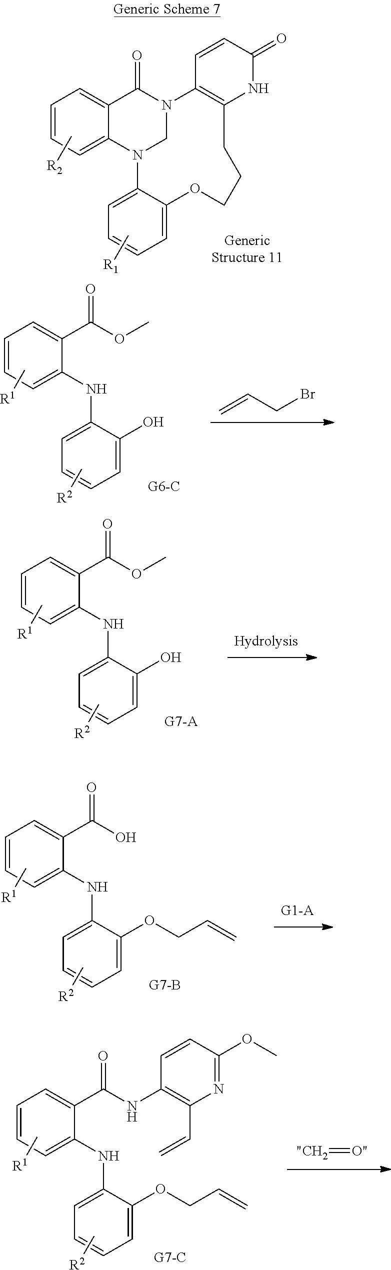

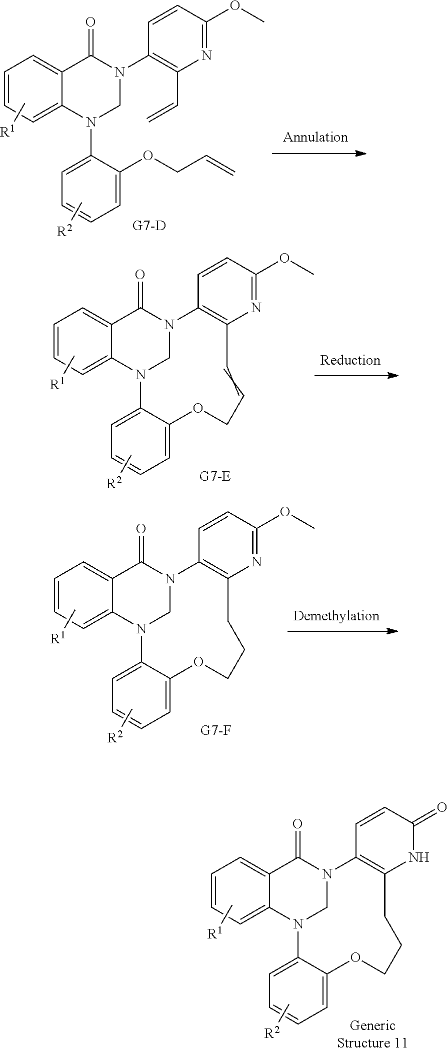

Compounds with Generic Structure 11 can be prepared by the general sequence outlined in Generic Scheme 7. Alkylation of intermediate G6-C can be accomplished using an inorganic base, such as potassium carbonate, and alkyl halide, such as allyl bromide, and the appropriate solvent, such as THF, acetone or DMF, at elevated temperature, followed by ester hydrolysis (G7-B), and amide coupling with G1-A to yield G7-C using aforementioned chemistries. Likewise, dihydropyrimidinone ring formation (G7-D), Grubbs-catalyzed annulation (G7-E), catalytic hydrogenation (G7-F) and demethylation to the pyridinone ring gives Generic Structure 11 using previously described conditions.

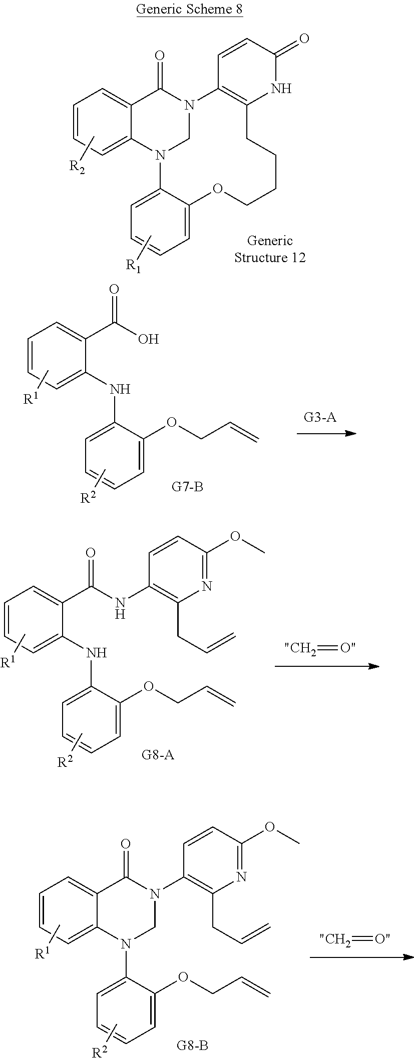

Compounds with Generic Structure 12 can be prepared by the general sequence outlined in Generic Scheme 8. Using the sequence of reaction conditions previously described, carboxylic acid G7-B can be coupled with amine G3-A to afford G8-A and subsequent formation of the dihydropyrimidinone ring (G8-B), Grubbs-catalyzed annulation (G8-C), catalytic hydrogenation (G8-D) and demethylation produces Generic Structure 12.

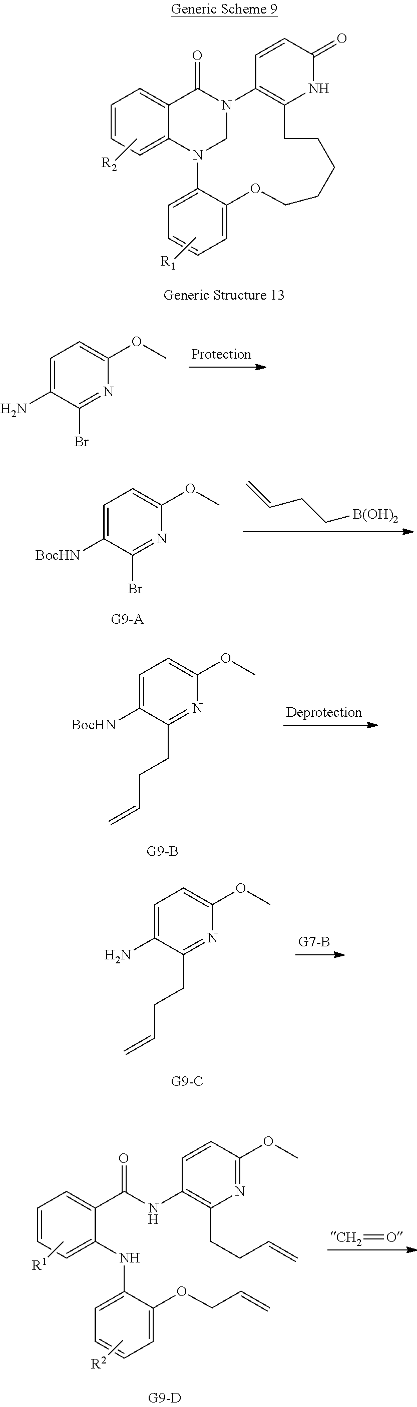

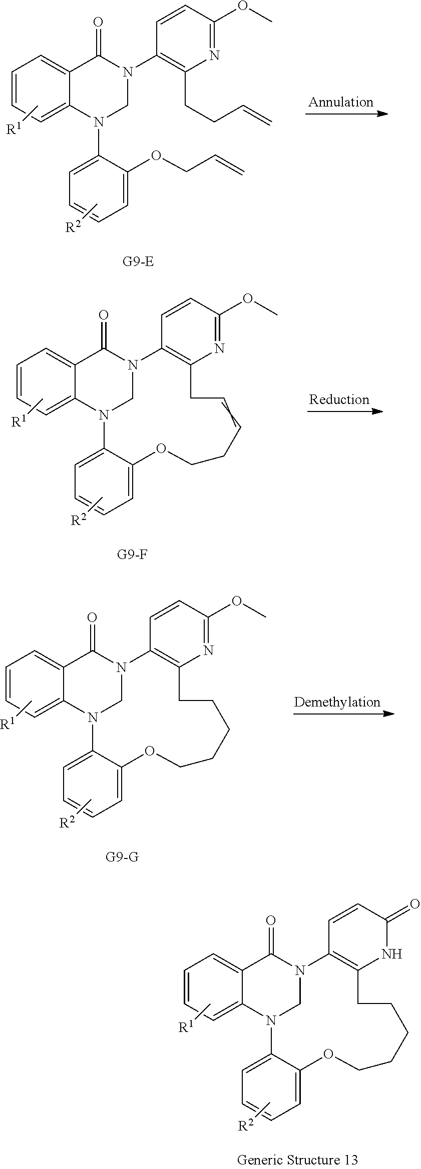

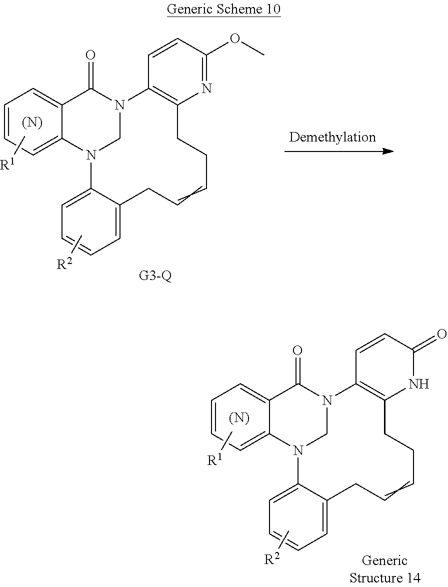

Compounds with Generic Structure 14 can be prepared by the general sequence outlined in Generic Scheme 10, in which intermediate G3-Q is demethylated with NaI/TMSCl in the appropriate solvent, such as acetonitrile, at elevated temperature, to produce Generic Structure 14.

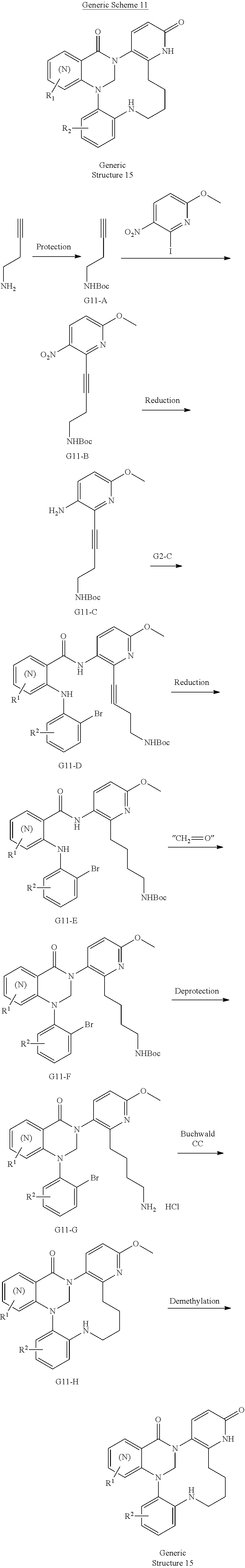

Compounds with Generic Structure 15 can be prepared by the general sequence outlined in Generic Scheme 11. Carbamate protection of aminobutyne (G11-A) followed by Sonogashira cross-coupling to 2-iodo-3-nitro-6-methoxypyridine forms G11-B. Nitro group reduction using a metal, such as zinc or iron, in the presence of a mild acid, such as ammonium chloride (G11-C), then amide coupling using previously described procedures with carboxylic acid G2-C gives G11-D. Using aforementioned procedures, catalytic hydrogenation of the alkyne (G11-E) formation of the dihydropyrimidinone (G11-F), deprotection of the amine (G11-G), buchwald cross-coupling to the macrocycle (G11-H), and demethylation to the pyridinone ring gives Generic Structure 15.

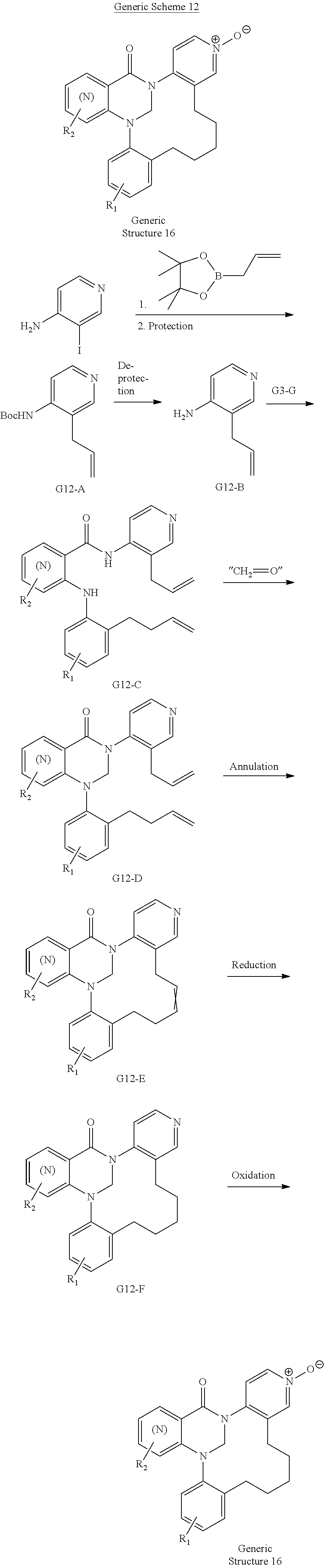

Compounds with Generic Structure 16 can be prepared by the general sequence outlined in Generic Scheme 12. Suzuki cross-coupling of 3-iodo-4-aminopyridine with propene pinacolborane, followed by N—BOC protection, using previously described methods, affords G12-A. Amine deprotection (G12-B), amide coupling with carboxylic acid G3-G gives G12-C, which is treated with paraformaldehyde and sulfuric acid to form the dihydropyrimidinone ring of G12-D, using aforementioned procedures. Annulation to the macrocycle (G12-D) followed by reduction of the olefin, can be achieved using conditions such as hydrazine/nosyl chloride, in the appropriate solvent, such as acetonitrile, at reduced temperature, gives G12-F. Oxidation to the N-oxide can be achieved using an oxidizing agent, such as m-CPBA, in a chlorinated solvent, such as dichloromethane or dichloroethane, at reduced temperature, to achieve Generic Structure 16.

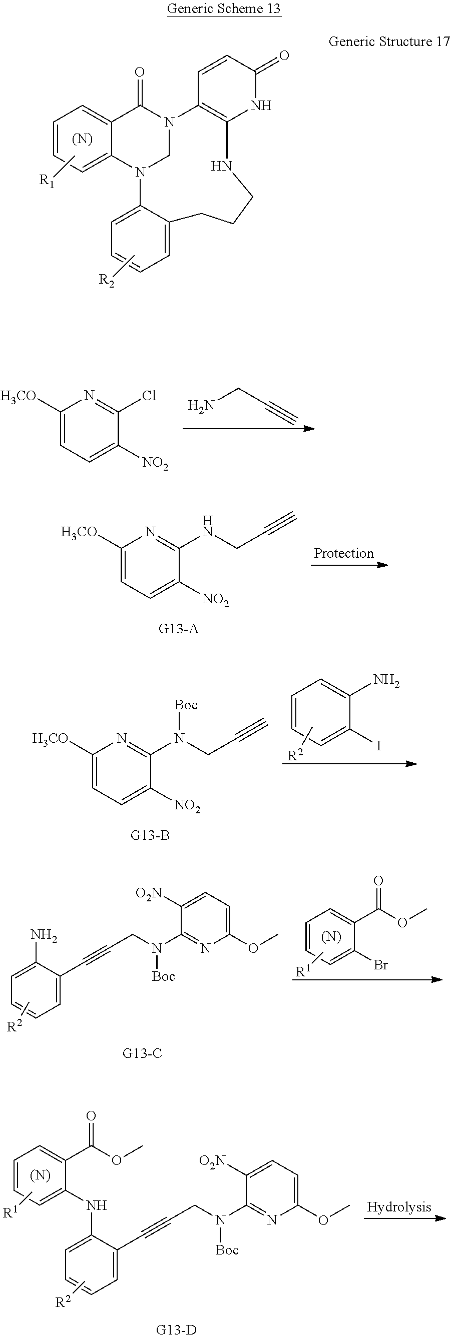

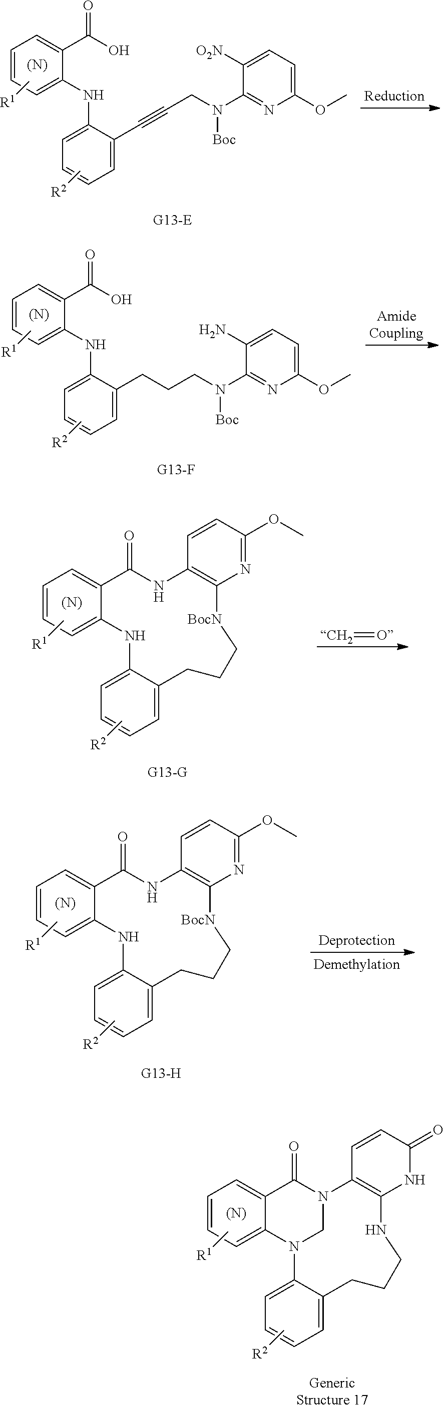

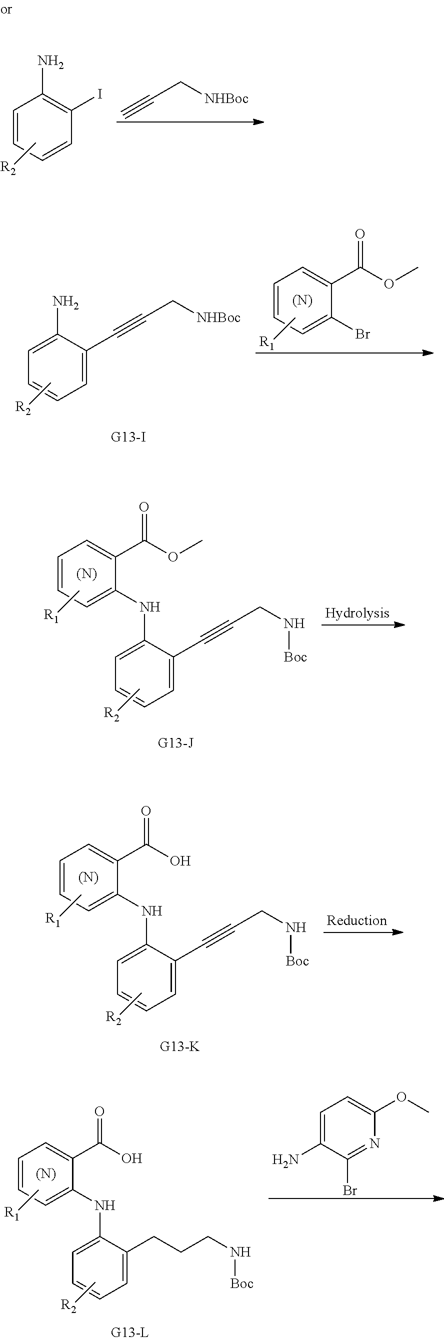

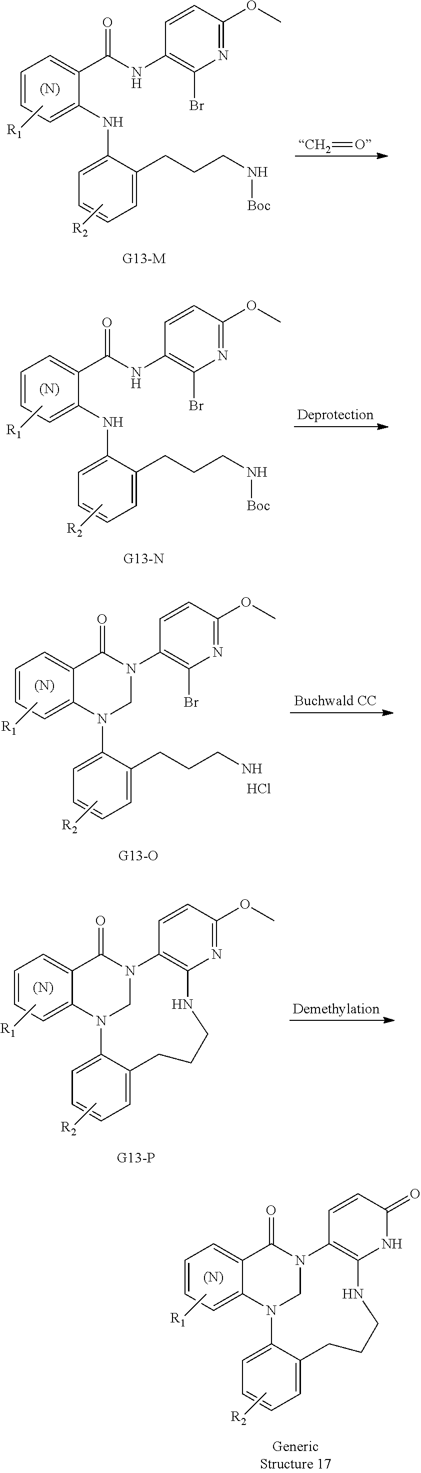

Compounds with Generic Structure 17 can be prepared by the general sequences outlined in Generic Scheme 13. Nucleophilic displacement of 2-chloro-6-methoxy-3-nitropyridine with aminopropyne in a solvent such as DMIF, NMP, or DMSO, and in the presence of a base, such as TEA or DIEA, at elevated temperature affords G13-A. N—BOC protection (G13-B) followed by and Sonogashira cross-coupling with R²-substituted o-iodoanilines using aforementioned methods affords G13-C. Subsequent buchwald cross-coupling with an R¹-substituted o-bromoarylcarbonates produces G13-D, saponification (G13-E), and catalytic hydrogenation (G13-F), intramolecular amide coupling (G13-G), dihydropyrimidinone ring formation (G13-H), and dual deprotection/demethylation under acidic conditions forms the pyridinone moiety to produce Generic Structure 17.

Alternatively, using previously described methods, R²-substituted o-iodoanilines can be subjected to a sonogashira cross-coupling with Boc-protected aminopropyne to afford G13-I, which subsequently undergoes a buchwald cross-coupling with R¹-substituted o-bromoarlycarboxylates (G13-J), followed by hydrolysis (G13-K), catalytic hydrogenation of the alkyne (G13-L), amide coupling (G13-M), dihydropyrimidinone formation (G13-N), amine deprotection (G13-0), an intramolecular buchwald cross-coupling (G13-P), and demethylation to the pyridinone to give Generic Structure 17.

Additionally, hydrogenation of the triple bond of intermediate G13-I to the alkane can be achieved with Pd(OH)₂, with the Scheme proceeding on with the omission of a subsequent hydrogenation.

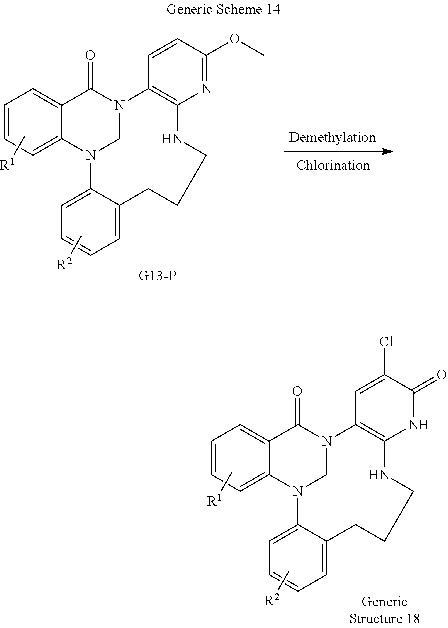

Compounds with Generic Structure 18 can be prepared by the general sequence outlined in Generic Scheme 14, in which G13-P is demethylated and subsequently chlorinated at elevated temperature to give Generic Structure 18.

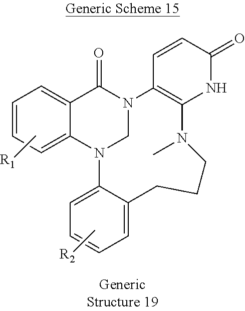

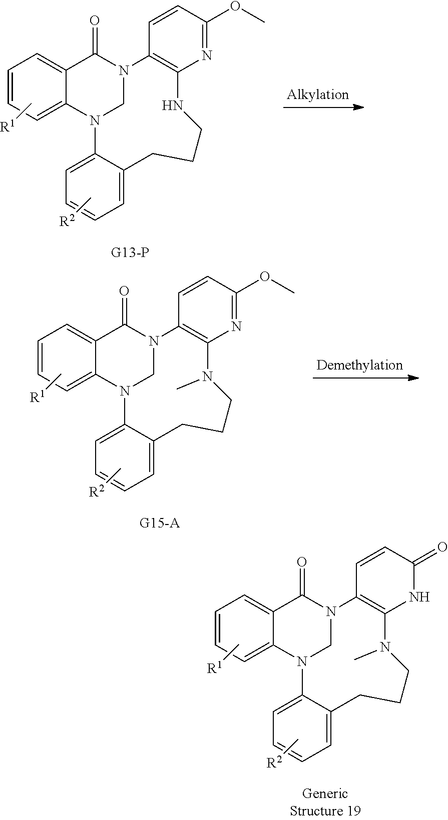

Compounds with Generic Structure 19 can be prepared by the general sequence outlined in Generic Scheme 15. Intermediate G13-P is alkylated using an alkyl halide, such as methyl iodide, and a base, such as sodium hydride, in the appropriate solvent, such as DMF or THF, followed by demethylation methods described previously to give Generic Structure 19.

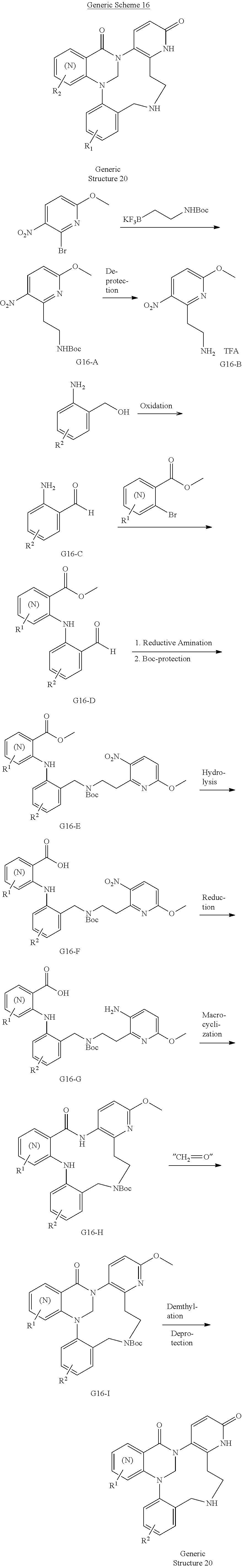

Compounds with Generic Structure 20 can be prepared by the general sequence outlined in Generic Scheme 16. Using aforementioned procedures, suzuki coupling of 2-bromo-3-nitro-6-methoxypyridine with BOC-amine-protected aminoethylboronate (G16-A) can be performed and then deprotected to yield G16-B. Concurrently, R²-substituted o-aminobenzyl alcohols can be oxidized to the corresponding aldehyde (G16-C) and then coupled via buchwald cross-coupling methods with R¹-substituted-bromoarylcarbonates to give G16-D, which subsequently can be reductively aminated with G16-B followed by Boc-amine protection to yield G16-E. Ester hydrolysis (G16-F) and reduction of the nitro group with previously described procedures produces G16-G, which can undergo an intramolecular amide coupling to macrocycle G16-H. Treatment under mild basic conditions with diiodomethane produces the dihydropyrimidinone G16-I, and demethylation under acidic condition results in amine deprotection and formation of the pyridinone ring for Generic Structure 20.

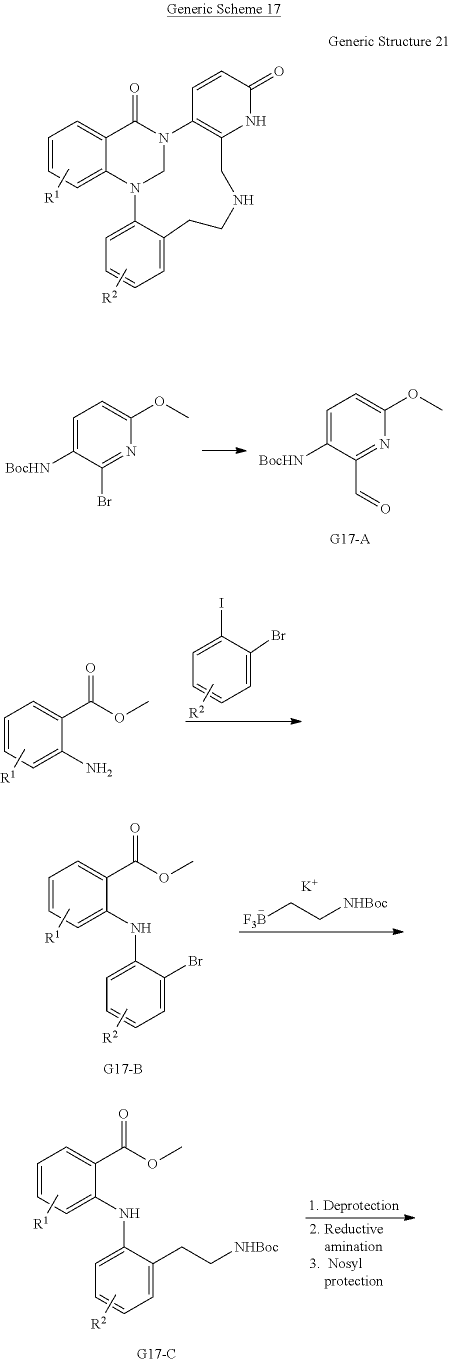

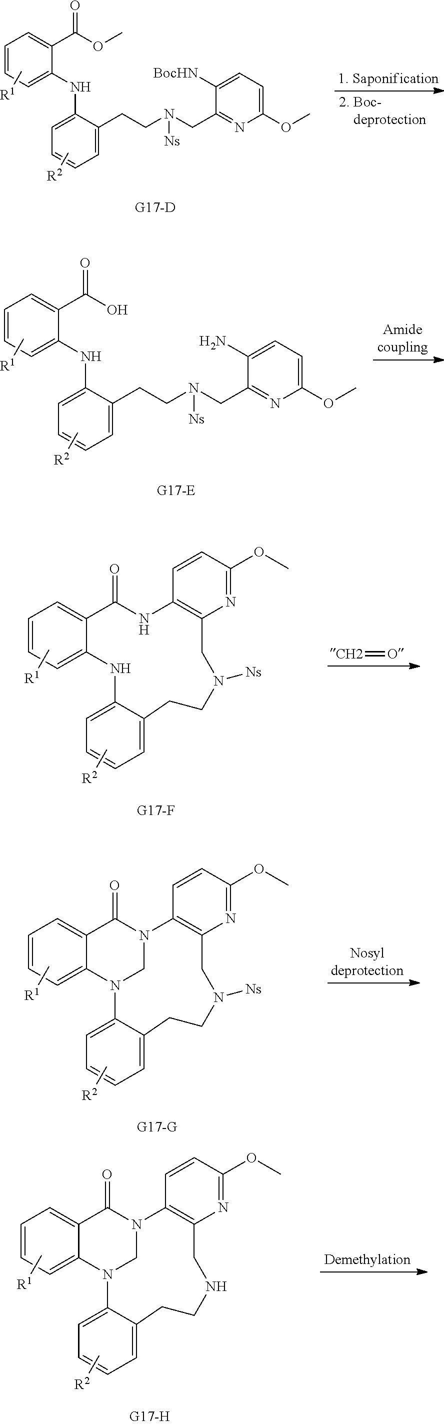

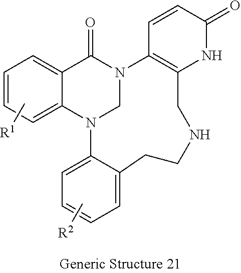

Compounds with Generic Structure 21 can be prepared by the general sequence outlined in Generic Scheme 17. tert-Butyl (2-bromo-6-methoxypyridin-3-yl)carbamate can be formylated by treatment with butyllithium and DMF to give the aldehyde G17-A. Concurrently, using aforementioned procedures, Buchwald coupling of an R¹-substituted o-aminobenzoate with an R² substituted 2-bromoiodobenzene can be performed (G17-B), followed by Suzuki coupling with boc-amine-protected aminoethylboronate to give G17-C. Boc-deprotection, reductive amination with G17-A and nosyl-protection produces G17-D. Ester hydrolysis and boc-deprotection (G17-E) enable intramolecular amide coupling, using previously described methods, to give G17-F. Dihydropyrimidinone formation (G17-G), nosyl-deprotection (G17-H) and demethylation to the pyridinone produces Generic Structure 21.

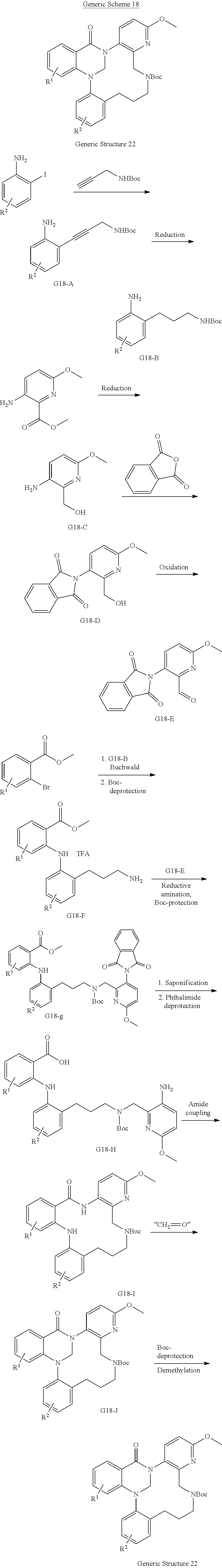

Compounds with Generic Structure 22 can be prepared by the general sequence outlined in Generic Scheme 18. R²-substituted o-iodoaniline undergoes Sonagashira coupling with tert-butyl prop-2-yn-1-ylcarbamate to form G18-A, which is catalytically hydrogenated to G18-B. Concurrently, methyl 3-amino-6-methoxypicolinate is reduced with LAH to alcohol G18-C, undergoes phthalimide protection to G18-D and finally Dess-Martin oxidation to aldehyde G18-E. Buchwald coupling of an R¹-substituted o-bromobenzoate with G18-B, followed by boc-deprotection gives the amine G18-F which undergoes reductive amination with G18-E to produce G18-G. Using previously discussed chemistry, hydrolysis of the ester followed by phthalimide deprotection using hydrazine produces G18-H, which undergoes intramolecular amide coupling (G18-I), dihydropyrimidinone formation (G18-J), and finally HCl-induced boc-deprotection and demethylation of the pyridone to produce Generic Structure 22.

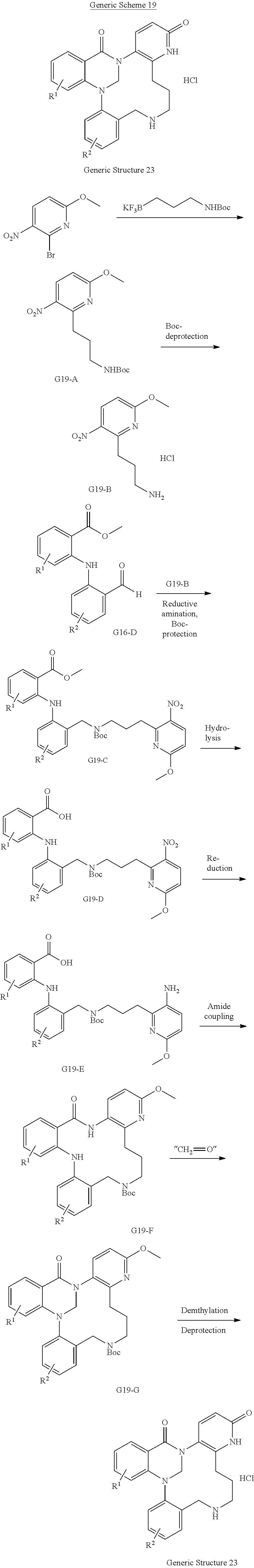

Compounds with Generic Structure 23 can be prepared by the general sequence outlined in Generic Scheme 19. 2-Bromo-6-methoxy-3-nitropyridine undergoes Suzuki coupling with tert-butyl (2-(trifluoro-)₄-boraneyl)propyl)carbamate, potassium salt to produce G19-A which is boc-deprotected to the amine G19-B. Concurrently, G16-D can be reductively aminated with G19-B followed by boc-amine protection to yield G19-C. Ester hydrolysis (G19-D) and reduction of the nitro group with previously described procedures produces G19-E, which can undergo an intramolecular amide coupling to macrocycle G19-F. Treatment under mild basic conditions with diiodomethane produces the dihydropyrimidinone G19-G, and demethylation under acidic condition results in amine deprotection and formation of the pyridinone ring for Generic Structure 23.

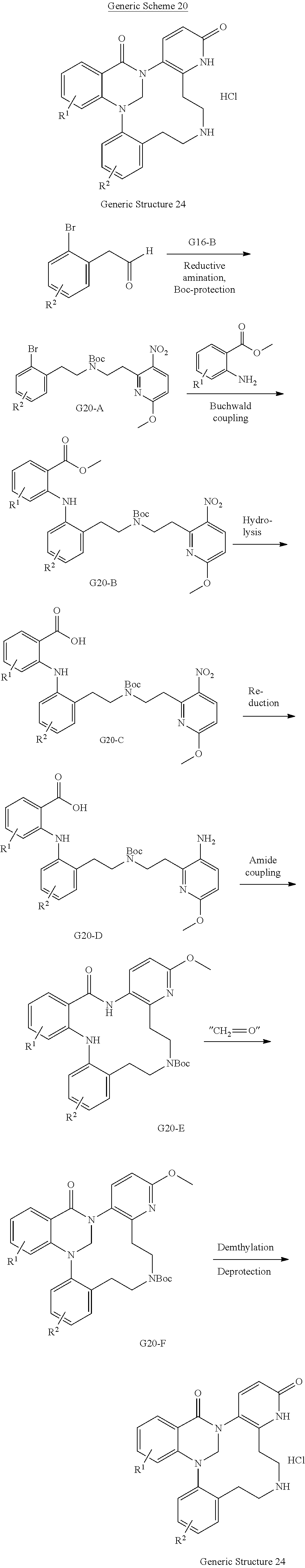

Compounds with Generic Structure 24 can be prepared by the general sequence outlined in Generic Scheme 20. R²-substituted 2-(2-bromophenyl)acetaldehyde can undergo reductive amination with G16-B followed by boc-protection to yield G20-A. Buchwald coupling to an R¹-substituted o-aminobenzoate gives G20-B. Ester hydrolysis (G20-C) and reduction of the nitro group with previously described procedures produces G20-D, which can undergo an intramolecular amide coupling to macrocycle G20-E. Treatment under mild basic conditions with diiodomethane produces the dihydropyrimidinone G20-F, and demethylation under acidic condition results in amine deprotection and formation of the pyridinone ring for Generic Structure 24.

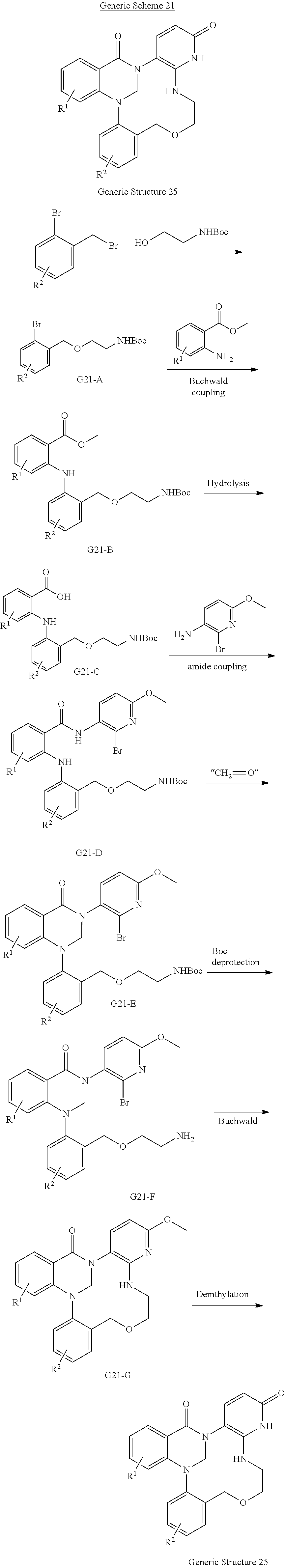

Compounds with Generic Structure 25 can be prepared by the general sequence outlined in Generic Scheme 21. R²-substituted o-bromobenzyl bromide can undergo nucleophilic displacement, under previously described conditions, with tert-butyl (2-hydroxyethyl)carbamate to give G21-A. Previously described Buchwald coupling to an R¹-substituted o-aminobenzoate gives G21-B. Also using previous chemistry, ester hydrolysis (G21-C), amide coupling with 2-bromo-6-methoxypyridin-3-amine (G21-D) followed by dihydropyrimidinone ring formation gives G21-E. Subsequent deprotection of the amine (G21-F), intramolecular ring closure (G21-G), and demethylation using previously described steps yield Generic Structure 25.

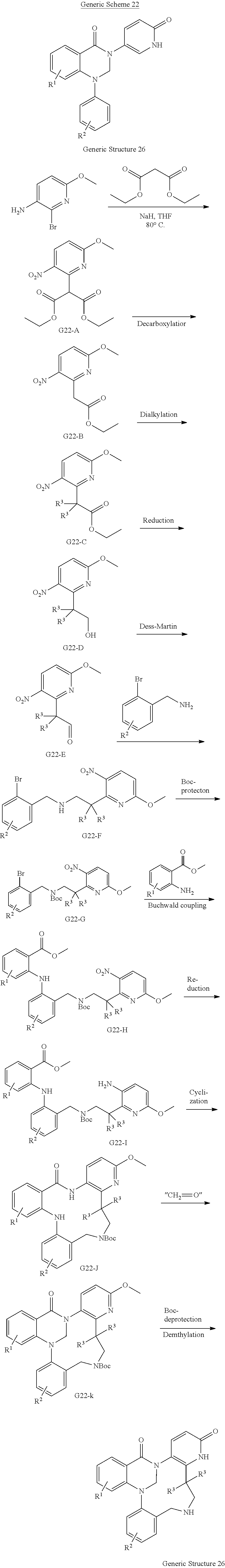

Compounds with Generic Structure 26 can be prepared by the general sequence outlined in Generic Scheme 22. Nucleophilic displacement of 2-bromo-6-methoxypyridin-3-amine with diethyl malonate in a solvent such as THF or DMF, and in the presence of a base such as NaH, affords G22-A. Decarboxylation in the presence of LiCl in DMSO/H₂O at elevated temperature produces G22-B. Dialkylation with an R-halide in the presence of base, such as NaH, in DMSO (G22-C), followed by reduction of the ester with a reducing agent such as LAH gives the alcohol G22-D. The aldehyde G22-E can be formed with a mild oxidizing agent such as Dess-Martin reagent, and can then undergo reductive amination with R²-substituted (2-bromophenyl)methanamine (G22-F), followed by boc-protection to yield G22-G. Buchwald cross-coupling methods with R¹-substituted o-bromoanilinocarbonates produce G22-H. Reduction of the nitro group using previously described procedures yields G22-I, which can then be treated with a base such as LiHMDS to achieve macrocyclization to G22-J. Treatment under mild basic conditions with diiodomethane produces the dihydropyrimidinone G22-K, and treatment with TMSI, in a solvent such as acetonitrile, at elevated temperature results in boc-deprotection and formation of the pyridinone ring for Generic Structure 26.

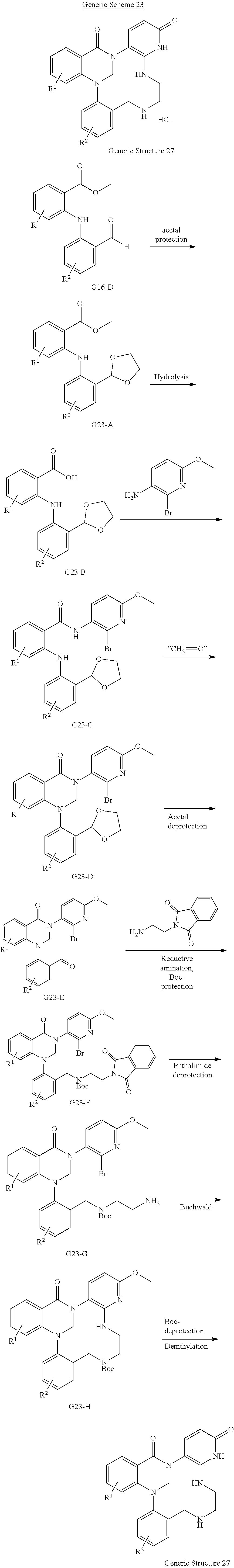

Compounds with Generic Structure 27 can be prepared by the general sequence outlined in Generic Scheme 23. G16-D is protected as the acetal via treatment with tosic acid and ethylene glycol (G23-A), then hydrolyzed (G23-B) and amide coupled to 2-bromo-6-methoxypyridin-3-amine (G23-C), using previously discussed chemistries. Treatment under mild basic conditions with diiodomethane produces the dihydropyrimidinone G23-D, which is acetal-deprotected with treatment of HCl in dioxane (G23-E), enabling reductive amination with 2-(2-aminoethyl)isoindoline-1,3-dione (G23-F) followed by boc-protection to give G23-G. Removal of the phthalimide protecting group (G23-H) enables macrocyclization via previously discussed Buchwald chemistry (G23-H) followed by demethylation under acidic condition resulting in amine deprotection and formation of the pyridinone ring for Generic Structure 27.

### Methods/Uses

In general, the invention also relates to uses of the compounds and/or pharmaceutical compositions described herein for use as a medicament or for use in therapy.

Compounds of the invention as defined herein are inhibitors of voltage-gated sodium ion channels, and particularly the voltage-gated sodium ion channel Naᵥ1.8. The activity of a compound utilized in this invention as an inhibitor of Nav1.8 can be assayed according to methods described generally in the Examples herein, or according to methods available to one of ordinary skill in the art.

In one aspect, the invention relates to uses of compounds and pharmaceutical compositions as described herein as inhibitors of voltage-gated sodium ion channels, particularly Naᵥ1.8.

In an embodiment, the invention relates to a method of inhibiting a voltage-gated sodium ion channel in a subject in need thereof, comprising administering to the subject an effective amount of a compound of the invention or a pharmaceutical composition of the invention as described herein. In another embodiment, the voltage-gated sodium channel is Naᵥ1.8.

In an embodiment, the invention relates to a compound of the invention or a pharmaceutical composition of the invention for use in inhibiting a voltage-gated sodium ion channel. In another embodiment, the voltage-gated sodium channel is Naᵥ1.8.

In an embodiment, the invention relates to use of a compound of the invention or a pharmaceutical composition of the invention in the manufacture of a medicament for inhibiting a voltage-gated sodium ion channel. In another embodiment, the voltage-gated sodium channel is Naᵥ1.8.

Without wishing to be bound by any particular theory, the compounds and compositions of the invention are particularly useful for treating a disease, condition, or disorder where activation or hyperactivity of Nav1.8 is implicated in the disease, condition, or disorder. When activation or hyperactivity of Nav1.8 is implicated in a particular disease, condition, or disorder, the disease, condition, or disorder may also be referred to as a “Nav1.8-mediated disease, condition or disorder.” Exemplary Nav1.8-mediated diseases, disorders, and conditions include pain and pain-associated diseases, and cardiovascular diseases, such as atrial fibrillation.

According to embodiments of the invention, a pain-associated disease is pain caused by any one of a variety of diseases of varying etiologies as described throughout the disclosure. In some embodiments, pain or a pain-associated disease is neuropathic pain, chronic pain, acute pain, nociceptive pain, inflammatory pain, musculoskeletal pain, visceral pain, cancer pain, idiopathic pain, multiple sclerosis, Charcot-Marie-Tooth syndrome, or incontinence.

In some embodiments, pain or a pain-associated disease is neuropathic pain or chronic neuropathic pain. In some embodiments, pain or a pain-associated disease is neuropathic pain or chronic neuropathic pain selected from small fiber neuropathy, small fiber-mediated diabetic neuropathy, idiopathic small fiber neuropathy, painful diabetic neuropathy or polyneuropathy.

In some embodiments pain or a pain-associated disease is neuropathic pain selected from post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion, complex regional pain syndrome, drug therapy induced neuralgia, cancer chemotherapy induced neuralgia, anti-retroviral therapy induced neuralgia, post spinal cord injury pain, idiopathic small-fiber neuropathy, idiopathic sensory neuropathy or trigeminal autonomic cephalalgia.

In some embodiments, pain or a pain-associated disease is neuropathic pain or chronic neuropathic pain selected from diabetic peripheral neuropathy, pain caused by neuropathy, neurologic or neuronal injury, pain associated nerve injury, neuralgias and associated acute or chronic pain, post-herpetic neuralgia, pain associated root avulsions, painful traumatic mononeuropathy, painful polyneuropathy, erythromelalgia, paroxysmal extreme pain disorder (PEPD), burning mouth syndrome, central pain syndromes caused by a lesion at a level of nervous system, traumatic nerve injury, nerve compression or entrapment, congenital insensitivity to pain (CIP), dysmenorrheal, primary erythromelalgia, HIV peripheral sensory neuropathy, pudendal neuralgia, spinal nerve injury, chronic inflammatory demyelinating polyneuropathy (CIDP), carpal tunnel syndrome and vasculitic neuropathy.

In some embodiments, pain or a pain-associated disease is visceral pain, wherein visceral pain is inflammatory bowel disease pain, Crohn's disease pain or interstitial cystitis pain.

In some embodiments, pain or a pain-associated disease is musculoskeletal pain, wherein musculoskeletal pain is osteoarthritis pain, back pain, cold pain, burn pain or dental pain.

In some embodiments, pain or a pain-associated disease is idiopathic pain, wherein idiopathic pain is fibromyalgia pain.

In some embodiments, pain or a pain-associated disease is chronic or acute pre-operative associated pain or chronic or acute post-operative associated pain. Post-operative associated pain includes ambulatory post-operative pain. Ambulatory surgery, also known as outpatient surgery, refers to same day surgery that does not require an overnight stay in a hospital or other medical facility. In some embodiments, pre-operative associated pain is selected from neuropathic pain or chronic neuropathic pain, chronic osteoarthritis pain, dental pain or inflammatory pain. In some embodiments, post-operative associated pain is selected from bunionectomy pain, hernia repair pair, breast surgery pain or cosmetic surgical pain.

In some embodiments, pain or a pain-associated disease is pain caused by trauma or iatrogenic medical or dental procedures. As used herein, the term “iatrogenic” refers to pain induced inadvertently by a medical or dental personnel, such as surgeon or dentist, during medical or dental treatment(s) or diagnostic procedure(s), which include, but are not limited to pain caused by pre-operative (i.e., “before”), peri-operative (i.e., “during” or medically induced pain during non-surgical or operative treatment(s)) and post-operative (i.e., after, post-operative or surgical induced caused pain) medical or dental procedures.

In some embodiments, pain or a pain-associated disease is nociceptive pain, wherein nociceptive pain is post-surgical pain, cancer pain, back and craniofacial pain, osteoarthritis pain, dental pain or diabetic peripheral neuropathy.

In some embodiments, pain or a pain-associated disease is inflammatory pain. Inflammatory pain can be pain of varied physiological origins. In some embodiments, inflammatory pain is selected from pain associated with osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization, complex regional pain syndrome, chronic arthritic pain and related neuralgias or acute pain. In some embodiments inflammatory pain is selected from pain associated with rheumatoid arthritis, osteoarthritis, rheumatoid spondylitis, gouty arthritis or juvenile arthritis. In some embodiments, inflammatory pain is selected from rheumatoid arthritis, rheumatoid spondylitis, gouty arthritis, juvenile arthritis, rheumatic disorder, gout, shoulder tendonitis or bursitis, polymyalgia rheumatica, primary hyperalgesia, secondary hyperalgesia, primary allodynia, secondary allodynia, or other pain caused by central sensitization, complex regional pain syndrome, chronic or acute arthritic pain and related neuralgias. In some embodiments, inflammatory pain is rheumatoid arthritis pain or vulvodynia.

In some embodiments, inflammatory pain is osteoarthritis, chronic osteoarthritis pain (e.g., hip or knee) or chronic inflammatory demyelinating polyneuropathy.

In some embodiments pain or a pain-associated disease is musculoskeletal pain. In some embodiments, musculoskeletal pain is selected from bone and joint pain, osteoarthritis, lower back and neck pain, or pain resulting from physical trauma or amputation. In some embodiments, musculoskeletal pain is selected from bone and joint pain, osteoarthritis (e.g., knee, hip), tendonitis (e.g., shoulder), bursitis (e.g., shoulder) tenosynovitis, lower back and neck pain, sprains, strains, or pain resulting from physical trauma or amputation.

In some embodiments, pain or a pain-associated disease is neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system), traumatic nerve injury, nerve compression or entrapment, congenital insensitivity to pain (CIP), dysmenorrheal, primary erythromelalgia; HIV peripheral sensory neuropathy, pudendal neuralgia, spinal nerve injury, chronic inflammatory demyelinating polyneuropathy (CIDP), carpal tunnel syndrome or vasculitic neuropathy.

In some embodiments, pain or a pain-associated disease is pain caused by trauma, or pain caused by iatrogenic, medical, or dental procedures.

In some embodiments, pain or a pain-associated disease is myofascial pain, myositis or muscle inflammation, repetitive motion pain, complex regional pain syndrome, sympathetically maintained pain, cancer, toxins and chemotherapy related pain, postsurgical pain syndromes and/or associated phantom limb pain, post-operative medical or dental procedures or treatments pain, or pain associated with HIV or pain induced by HIV treatment.

In some embodiments, pain or a pain-associated disease, disorder, or condition is neuropathic pain or other pain-associated disease selected from peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, or chronic inflammatory conditions.

In some embodiments, pain or a pain-associated disease is acute pain, chronic pain, neuropathic pain, inflammatory pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, dipolar disorder, myotonia, arrhythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head pain, neck pain, severe pain, intractable pain, nociceptive pain, breakthrough pain, postsurgical pain, cancer pain, stroke, cerebral ischemia, traumatic brain injury, amyotrophic lateral sclerosis, stress induced angina, exercise induced angina, palpitations, hypertension, or abnormal gastro-intestinal motility.

In some embodiments, pain or a pain-associated disease is femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache (including cluster headaches), chronic and acute neuropathic pain, post-herpetic neuralgia, diabetic neuropathy, HIV-associated neuropathy, trigeminal neuralgia, Charcot-Marie Tooth neuropathy, hereditary sensory neuropathies, peripheral nerve injury, painful neuromas, ectopic proximal and distal discharges, radiculopathy, chemotherapy induced neuropathic pain, radiotherapy-induced neuropathic pain, post-mastectomy pain, central pain, spinal cord injury pain, post-stroke pain, thalamic pain, complex regional pain syndrome, phantom pain, intractable pain, acute pain, acute post-operative pain, acute musculoskeletal pain, joint pain, mechanical low back pain, neck pain, tendonitis, injury/exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory, burn and trauma pain, acute intermittent pain, endometriosis, acute herpes zoster pain, sickle cell anemia, acute pancreatitis, breakthrough pain, orofacial pain including sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, including, urinary incontinence, hyperactivity bladder, painful bladder syndrome, interstitial cyctitis (IC), prostatitis, complex regional pain syndrome (CRPS) (type I and type II), widespread pain, paroxysmal extreme pain, pruritis, tinnitus, or angina-induced pain.

In another aspect, the invention relates to uses of compounds and pharmaceutical compositions of the invention in methods and medicaments for treating cardiovascular diseases, including atrial fibrillation and cardiac arrhythmias.

In some embodiments, a cardiovascular disease is atrial fibrillation that is either idiopathic in nature or caused by a disease as defined herein. Atrial fibrillation can be paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease. In particular embodiments, atrial fibrillation is selected from paroxysmal, sustained, or long-standing atrial fibrillation.

In some embodiments, a cardiovascular disease includes cardiac arrhythmias.

Thus, in another aspect, the invention also provides a method of treatment in a subject, especially a human. Disease states which can be treated by the methods and compositions provided herein include, but are not limited to, pain and pain associated diseases, and cardiovascular diseases.

The term “treatment” refers to alleviating the specified condition, eliminating or reducing one or more symptoms of the condition, slowing or eliminating the progression of the condition, and delaying the reoccurrence of the condition in a previously afflicted patient or subject.

As used herein, “effective amount” and “therapeutically effective amount” are used interchangeably. The term “therapeutically effective amount” refers to the quantity of a compound of formula (I-a), (I), (II), (III), (IV), or (V), or a tautomer thereof, or a pharmaceutically acceptable salt thereof, which will elicit the desired biological response in the human body. It may vary depending on the compound, the disease and its severity, and the age and weight of the subject to be treated.

The term “subject” refers to a human body.

In one aspect, the invention relates to a method of treatment of pain or a pain-associated disease as defined herein in a human in need thereof, comprising administering to the human a compound of the invention or a pharmaceutical composition of the invention as described herein.

In an embodiment, provided is a method of treatment of acute pain or chronic pain in a human in need thereof, comprising administering to the human a compound of the invention or a pharmaceutical composition of the invention as described herein.

In an embodiment, provided is a method of treatment of pain caused by trauma, pain caused by iatrogenic medical or dental procedures, or pre-operative or post-operative associated pain in a human in need thereof, comprising administering to the human a compound of the invention or a pharmaceutical composition of the invention as described herein.

In an embodiment, provided is a method of treatment of neuropathic pain, nociceptive pain, inflammatory pain, musculoskeletal pain, visceral pain, or idiopathic pain in a human in need thereof, comprising administering to the human a compound of the invention or a pharmaceutical composition of the invention as described herein.

In an embodiment, provided is a method of treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy in a human in need thereof, comprising administering to the human a compound of the invention or a pharmaceutical composition of the invention as described herein.

In an embodiment, provided is a method of treatment of inflammatory pain selected from the group consisting of osteoarthritis, chronic osteoarthritis pain, and chronic inflammatory demyelinating polyneuropathy in a human in need thereof, comprising administering to the human a compound of the invention or a pharmaceutical composition of the invention as described herein.

In an embodiment, provided is a method of treatment of a pain or a pain-associated disease selected from the group consisting of neuropathic pain, ambulatory post-operative pain, and osteoarthritis in a human in need thereof, comprising administering to the human a compound of the invention or pharmaceutical composition of the invention as described herein.

In some embodiments, the pain or pain-associated disease is neuropathic pain. In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In another aspect, the invention provides compounds of the invention and pharmaceutical compositions of the invention as described herein for use in treatment of pain or a pain-associated disease as defined herein.

In an embodiment, provided is a compound of the invention or pharmaceutical composition of the invention for use in treatment of acute pain or chronic pain.

In an embodiment, provided is a compound of the invention or pharmaceutical composition of the invention for use in treatment of pain caused by trauma, pain caused by iatrogenic medical or dental procedures, or pre-operative or post-operative associated pain.

In an embodiment, provided is a compound of the invention or pharmaceutical composition of the invention for use in treatment of neuropathic pain, nociceptive pain, inflammatory pain, musculoskeletal pain, visceral pain, or idiopathic pain.

In an embodiment, provided is a compound of the invention or pharmaceutical composition of the invention for use in treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy.

In an embodiment, provided is a compound of the invention or pharmaceutical composition of the invention for use in treatment of inflammatory pain selected from the group consisting of osteoarthritis, chronic osteoarthritis pain, and chronic inflammatory demyelinating polyneuropathy.

In an embodiment, provided is a compound of the invention or pharmaceutical composition of the invention for use in treatment of pain or a pain-associated disease selected from the group consisting of neuropathic pain, ambulatory post-operative pain, and osteoarthritis.

In some embodiments, the pain or pain-associated disease is neuropathic pain. In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In another aspect, the invention also provides uses of compounds of the invention or pharmaceutical compositions of the invention as described herein in the manufacture of a medicament for treatment of pain and pain associated diseases as described herein.

In an embodiment, provided is use of a compound of the invention or pharmaceutical composition of the invention in the manufacture of a medicament for treatment of acute pain or chronic pain.

In an embodiment, provided is use of a compound of the invention or pharmaceutical composition of the invention in the manufacture of a medicament for treatment of pain caused by trauma, pain caused by iatrogenic medical or dental procedures, or pre-operative or post-operative associated pain.

In an embodiment, provided is use of a compound of the invention or pharmaceutical composition of the invention in the manufacture of a medicament for treatment of neuropathic pain, nociceptive pain, inflammatory pain, musculoskeletal pain, visceral pain, or idiopathic pain.

In an embodiment, provided is use of a compound of the invention or pharmaceutical composition of the invention in the manufacture of a medicament for treatment of neuropathic pain or chronic neuropathic pain selected from the group consisting of small fiber neuropathy, small fiber-mediated diabetic neuropathy, idiopathic small fiber neuropathy, painful diabetic neuropathy and polyneuropathy.

In an embodiment, provided is use of a compound of the invention or pharmaceutical composition of the invention in the manufacture of a medicament for treatment of inflammatory pain selected from the group consisting of osteoarthritis, chronic osteoarthritis pain, and chronic inflammatory demyelinating polyneuropathy.

In an embodiment, provided is use of a compound of the invention or pharmaceutical composition of the invention in the manufacture of a medicament for treatment of pain or a pain-associated disease selected from the group consisting of neuropathic pain, ambulatory post-operative pain, and osteoarthritis. In some embodiments, the pain or pain-associated disease is neuropathic pain. In some embodiments, the pain or pain-associated disease is chronic neuropathic pain. In some embodiments, the pain or pain-associated disease is small fiber neuropathy. In some embodiments, the pain or pain-associated disease is ambulatory post-operative pain. In some embodiments, the pain or pain-associated disease is osteoarthritis. In some embodiments, the pain or pain-associated disease is osteoarthritis of the knee and/or osteoarthritis of the hip.

In one aspect, the invention relates to a method of treatment of atrial fibrillation as defined herein in a human in need thereof, comprising administering to the human a compound of the invention or a pharmaceutical composition of the invention as described herein. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to a compound of the invention or a pharmaceutical composition of the invention for use in treatment of atrial fibrillation. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to use of a compound of the invention or a pharmaceutical composition of the invention as described herein in the manufacture of a medicament for treatment of atrial fibrillation. In some embodiments, the atrial fibrillation is selected from the group consisting of paroxysmal atrial fibrillation, sustained atrial fibrillation, long-standing atrial fibrillation, atrial fibrillation with heart failure, atrial fibrillation with cardiac valve disease, and atrial fibrillation with chronic kidney disease.

In another aspect, the invention relates to a compound of the invention or a pharmaceutical composition of the invention as described herein for use in therapy.

### Combination Therapy

The compounds and pharmaceutical compositions of the invention disclosed herein can be combined with or co-administered with other therapeutic agents, particularly agents that may enhance the activity or time of disposition of the compounds. Combination therapies according to the invention comprise the administration of at least one compound of the invention and the use of at least one other treatment method, including administration of one or more other therapeutic agents.

By the term “co-administration” and derivatives thereof as used herein refers to either simultaneous administration or any manner of separate sequential administration of a Naᵥ1.8 inhibiting compound of the invention, as described herein, and an additional active ingredient. An additional active ingredient includes any compound or therapeutic agent known to or that demonstrates advantageous properties when administered to a human in need of treatment. Typically, if the administration is not simultaneous, the compounds are administered in a close time proximity to each other. Furthermore, the compounds may be administered in the same or separate dosage form, e.g., one compound may be administered orally and another compound may be administered intravenously.

Other therapeutic agents which may be used in combination with a compound of the invention include, but are not limited to Acetaminophen, Acetylsalicylic acid, Nav1.7 Inhibitors, Nav1.9 Inhibitors, anti-depressants (i.e. such as, but not limited to duloxetine or amitriptyline), anti-convulsants (i.e. such as, but not limited to pregabalin and gabapentin), opiates (i.e., such as, but not limited to hydrocodone, codeine, morphine, oxycodone, oxymorphone, fentanyl, and the like), etc.; and where administration of the above, respectively, also is determined by one of ordinary skill in the art. In one aspect, suitable Nav1.7 Inhibitors or Nav1.9 Inhibitors for use in the invention, include, but are not limited to those Nav1.7 Inhibitors or Nav1.9 Inhibitors known in the chemical literature.

Each component of a combination used for therapeutic purposes (e.g., compound or pharmaceutical composition of the invention and additional therapeutic agent) may be administered orally, intravenously or parenterally or in combinations thereof. Each component of a therapeutic combination may be, but is not limited to being administered by simultaneous administration, co-administration, or serial administration; and/or by identical or different routes of administration or combinations of administration routes. In certain embodiments, each identical or different route of administration or combinations of administration routes is selected from oral, intravenous or parenteral administration.

### EXAMPLES

The following examples illustrate the invention. These examples are not intended to limit the scope of the invention, but rather to provide guidance to the skilled artisan to prepare and use the compounds, compositions, and methods of the invention. While embodiments of the invention are described, the skilled artisan will appreciate that various changes and modifications can be made without departing from the spirit and scope of the invention.

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The invention is intended to encompass such salts.

Final compounds were characterized with LCMS (conditions listed below) and NMR. ¹H NMR or ¹⁹FNMR spectra were recorded using a BrukerAvance III 500 MHz spectrometer, BrukerAvance 400 MHz spectrometer and Varian Mercury Plus-300 MHz spectrometer. CDCI₃ is deuteriochloroform, DMSOd₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Unless otherwise noted, all starting materials were obtained from commercial suppliers and used without further purification. Unless otherwise indicated, all temperatures are expressed in 0° C. (degrees Centigrade). Unless otherwise indicated, all reactions are conducted under an inert atmosphere at ambient temperature. All temperatures are given in degrees Celsius, all solvents are highest available purity and all reactions run under anhydrous conditions in an argon (Ar) or nitrogen (N₂) atmosphere where necessary.

### Instrumentation

¹H NMR spectra were recorded using a BrukerAvance III 400 MHz spectrometer, BrukerAvance NEO NanoBay V4-3 400 MHz spectrometer. CDCI₃ is deuteriochloroform, DMSO-d₆ is hexadeuterio dimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Mass spectra were run on open access LC-MS systems, Waters Acquity QDa mass detector. The compound is analyzed using a reverse phase column, e.g., Xbridge-C₁₈, Sunfire-C₁₈₈, Thermo Aquasil/Aquasil C₁₈, Acquity HPLC C₁₈, Thermo Hypersil Gold eluted using an acetonitrile and water gradient with a low percentage of an acid modifier such as 0.02% TFA.

### HPLC Methods

Method A: UPLC: Waters Acquity equipped with an Acquity CSH, C₁₈ (2.1 mm×30 mm, 1.7 μm column) using a gradient of 1-100% MeCN/H₂O/0.1% TFA over 1.85 min at 1.3 mL/min flow rate. Mass determinations were conducted using an Agilent 6110 Quadrupole MS with positive ESI;

Method B: UPLC: Waters Acquity equipped with an Acquity CSH, C₁₈ (2.1 mm×30 mm, 1.7 μm column) using a gradient of 1-100% MeCN/H₂O/0.1% 10 mM NH₄HCO₃ in water adjusted to pH 10 with 25% aq NH₄OH, over 1.85 min at 1.3 mL/min flow rate. Mass determinations were conducted using an Agilent 6110 Quadrupole MS with positive ESI;

Method C: HPLC: Agilent 1290 Infinity II equipped with a CSH, C_{18, 2.1×30} mm, 1.7 μm column, using a gradient of 3-97% CH₃CN/H₂O/0.1% HCO₂H over 2.0 min. at 1.0 mL/min flow rate. Mass determinations were conducted using an Agilent 6110 Quadrupole MS with positive ESI;

Method D: HPLC: Agilent 1290 Infinity II equipped with a XBRIDGE, C_{8, 4.6×50} mm, 3.5 μm column, using a gradient of 5-95% CH₃CN/H₂O/0.1% NH₄HCO₃ over 6.0 min. at 1.2 mL/min flow rate. Mass determinations were conducted using an Agilent 6110 Quadrupole MS with positive ESI;

Method E: HPLC: Agilent 1290 Infinity II equipped with a CSH, C_{18, 2.1×30} mm, 3.5 μm column, using a gradient of 0-100% CH₃CN/H₂O/0.1% HCO₂H over 4.0 min. at 1.0 mL/min flow rate. Mass determinations were conducted using an Agilent 6110 Quadrupole MS with positive ESI;

Method F: HPLC: Agilent 1290 Infinity II equipped with a Sunfire, C_{18, 2.1×30} mm, 3.5 μm column, using a gradient of 0-100% CH₃CN/H₂O/0.1% HCO₂H over 4.0 min. at 1.0 mL/min flow rate. Mass determinations were conducted using an Agilent 6110 Quadrupole MS with positive ESI;

Method G: UPLC: Waters Acquity equipped with an Acquity CSH, C₁₈ (2.1 mm×30 mm, 1.7 μm column) using a gradient of 1-100% MeCN/H₂O/0.1% HCO₂H over 1.85 min at 1.3 mL/min flow rate. Mass determinations were conducted using an Agilent 6110 Quadrupole MS with positive ESI;

Method H: HPLC: Shimadzu LC-20AB equipped with a Kinetex, C₁₈ (2.1 mm×50 mm, 5 μm column) using a gradient of 30-90% MeCN/H₂O/0.10% TFA over 5.4 min at 0.5 mL/min flow rate.

Method I HPLC: Agilent 1260 Infinity II equipped with a Luna, C_{18, 2.0×50} mm, 5 μm column, using a gradient of 5-95% CH₃CN (0.02% TFA)/H₂O (0.04% TFA) over 4.5 min. at 1.0 mL/min flow rate. Mass determinations were conducted using an Agilent 6110 single quadrupole MS with positive ESI;

### Example Definitions and Abbreviations

In the following experimental descriptions, the following abbreviations may be used:

| Abbreviation | Meaning |
|---|---|
| MeCN or CH₃CN | acetonitrile |
| AcOH | acetic acid |
| aq. | aqueous |
| ATM or atm | standard atmosphere |
| BBr₃ | boron tribromide |
| BH₃ | borane |
| BINAP | 2,2′-bis(diphenylphosphino)-1,1′-binaphthalene |
| Boc₂O | boc-anhydride |
| Bn | benzyl |
| Br₂ | bromine |
| Brine | saturated aqueous sodium chloride |
| BuLi or nBuLi | butyllithium |
| CBr₄ | carbon tetrabromide |
| CDI | carbonyldiimidazole |
| CH₂I₂ | diiodomethane |
| CH₃I | iodomethane |
| CH₂CI₂ or DCM | methylene chloride |
| Cs₂CO₃ | cesium carbonate |
| CsF | cesium fluoride |
| DBU | 1,8-diazabicyclo[5.4.0]undec-7-ene |
| DCE | 1,2-dichloroethane |
| DEAD | diethyl azodicarboxylate |
| DIPEA, DIEA, | N,N-diisopropylethylamine |
| Hunig's base | |
| DMA | dimethylacetamide |
| DMAP | 4-dimethylaminopyridine |
| DMF | N,N-dimethylformamide |
| DME | dimethoxyethane |
| DMSO | dimethylsulfoxide |
| EDC | 1-[3-(dimethylamino)propyl]-3-ethylcarbodiimide |
| | hydrochloride |
| Et | ethyl |
| Et₃N or TEA | triethylamine |
| Et₂O | diethyl ether |
| EtOAc | ethyl acetate |
| EtOH | ethanol |
| equiv. | equivalents |
| g, G, gm, GM | gram |
| GC/MS | gas chromatography-mass spectrometry |
| Greencat-iPr | (1,3-bis(2,6-diisopropylphenyl)imidazolidin-2- |
| | ylidene)dichloro(2-((1-(methoxy(methyl)amino)-3- |
| | methyl-1-oxobutan-2-yl)oxy) |
| | benzylidene)ruthenium(II) |
| h or hr | hour (s) |
| H₂ | hydrogen |
| H₂O | water |
| H₂SO₄ | sulfuric acid |
| HATU | (1-[bis(dimethylamino)methylene]-1H-1,2,3- |
| | triazolo[4,5-b]pyridinium3-oxide hexafluorophosphate |
| HBr | hydrobromic acid |
| HCI | hydrochloric acid |
| HCO₂H | formic acid |
| HOBt or HOBT | 1-hydroxybenzotriazole |
| HPLC | high performance liquid chromatography |
| Int. | intermediate |
| iPrOH or iPA | 2-isopropanol |
| K₂CO₃ | potassium carbonate |
| kg | kilogram |
| kg/cm² | kilogram per centimeter square |
| KHSO₄ | potassium hydrogen sulfate |
| KOAc | potassium acetate |
| K₃PO₄ | potassium phosphate |
| L or I | liter |
| LCMS | liquid chromatography-mass spectroscopy |
| LiCl | lithium chloride |
| LiOH | lithium hydroxide |
| LHMDS | lithium bis(trimethylsilyl)amide |
| mCPBA or | meta-chIoroperoxybenzoic acid |
| m-CPBA | |
| MDAP | mass directed auto purification |
| M | molar |
| Me | methyl |
| MeOH | methanol |
| mg, MG | milligram |
| MgSO₄ | magnesium sulfate |
| min or mins | minute (s) |
| ml or mL or ML | milliliter |
| Mmol or mmol | millimole |
| MnO₂ | manganese dioxide |
| MS | mass spectrum |
| μw | microwave |
| N₂ | nitrogen |
| NaBH₄ | sodium borohydride |
| Na(CN)BH₃ | sodium cyanoborohydride |
| NaCI | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| Nal | sodium iodide |
| NaOH | sodium hydroxide |
| Na₂SO₃ | sodium sulfite |
| Na₂S₂O₃ | sodium thiosulfate |
| Na₂SO₄ | sodium sulfate |
| NBS | N-bromosuccinimide |
| NH₄CI | ammonium chloride |
| NH₄CO₃ | ammonium carbonate |
| HCO₂•NH₄ | ammonium formate |
| NH₄OH | ammonium hydroxide |
| NiCl₂(DME) | nickel(II) chloride ethylene glycol dimethyl ether |
| | complex |
| nm | nanometer |
| NMO | 4-methylmorpholine N-oxide |
| NMP | N-methyl-2-pyrrolidone |
| NMR | nuclear magnetic resonance |
| pet. | petroleum ether |
| Pd/C or Pd-C | palladium on carbon |
| PdCI₂(dbpf) | 1,1′-bis(di-tert-butylphosphino)ferrocene |
| | dichloropalladium |
| Pd(dppf)CI₂/ | [1,1′-bis(diphenylphosphino)ferrocene]dichloro- |
| PdCI₂(dppf) | palladium(ll) |
| PdCI₂(dppf)- | [1,1′- |
| CH₂CI₂ adduct | bis(diphenylphosphino)ferrocene]dichloro- |
| | palladium(ll), complex with dichloromethane |
| PdCI₂(Xantphos) | dichloro[9,9-dimethyl-4,5- |
| | bis(diphenylphosphino)xanthene] palladium(ll) |
| Pd₂(dba)₃ | tris(dibenzylideneacetone)dipalladium(0) |
| Pd(Ph₃)₄, tetrakis | tetrakis(triphenylphosphine)palladium(0) |
| Pd(OAc)₂ | palladium acetate or PaIIadium(II) acetate |
| Pd(OH)₂ | palladium hydroxide |
| PL HCO₃ MP | macroporus polystyrene supported carbonate |
| PPh₃ | triphenylphosphine |
| pTSOH or PTSA | p-toluenesulfonic acid |
| or pTsOH | |
| pyoxim | [ethyl cyano(hydroxyimino)acetato-O₂]tri-1- |
| | pyrrolidinyl phosphonium hexafluorophosphate |
| rt or RT | room temperature or retention time (when use with |
| | chromatography) |
| sat'd | saturated |
| SFC | supercritical fluid chromatography |
| Si | silica |
| Si SPE | silica gel cartridges |
| SiO₂ | silica gel |
| sol'n | solution |
| TBME | tert-butylmethyl ether |
| tBuXphos | 2-di-tert-butylphosphino-2′,4′,6′-triisopropylbiphenyl |
| TEA | triethylamine |
| TFA | trifluoroacetic acid |
| THF | tetrahydrofuran |
| TMS | trimethylsilyl |
| TMS-Br or | trimethylsilyl bromide |
| TMSBr | |
| TMS-CI or | trimethylsilyl chloride |
| TMSCI | |
| TMSI | iodotrimethylsilane or trimethylsilyl iodide |
| rt | retention time |
| μM | micromolar |
| UPLC | ultra performance liquid chromatography |
| Xantphos | 4,5-bis(diphenylphosphino)-9,9-dimethylxanthene |
| Xphos | 2-dicyclohexylphosphino-2′,4′,6′-triisopropylbiphenyl |
| Zn | zinc dust |

### Intermediates

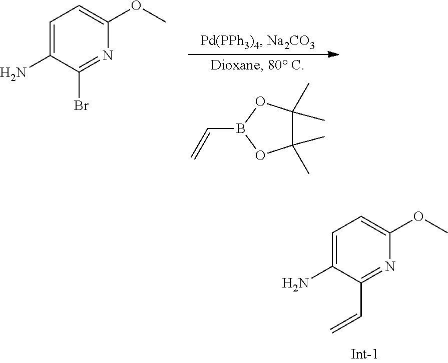

### Step A: 6-Methoxy-2-vinylpyridin-3-amine Int-i

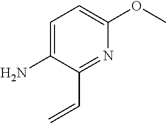

A solution of 2-bromo-6-methoxypyridin-3-amine (13.0 g, 64.0 mmol), 4,4,5,5-tetramethyl-2-vinyl-1,3,2-dioxaborolane (10.9 mL, 64.0 mmol) in 1,4-dioxane (160 mL) and aq. sat'd Na₂CO₃ (20.0 mL, 64.0 mmol) was purged with N₂ for 5 min, Pd(PPh₃)₄ (3.70 g, 3.20 mmol) was added under N₂ atmosphere, and the reaction mixture was heated at 80° C. for 16 h. The reaction mixture was allowed to cool to ambient temperature, quenched with H₂O (300 mL) and extracted with EtOAc (2×200 mL). The combined organic extracts were washed with H₂O (500 mL), brine, dried over Na₂SO₄, filtered and the solvent evaporated under reduced pressure. The crude residue was dissolved in DCM, preabsorbed onto a silica gel and purified by silica gel flash column chromatography (330 g) eluting with 15-17% EtOAc-pet. ether. Product fractions were combined and evaporated under reduced pressure to afford 6-methoxy-2-vinylpyridin-3-amine Int-1 (5.50 g, 56% yield) as a brown oil. HPLC/MS 0.371 min (C), [M+H]⁺ 151.2. ¹H NMR (DMSO-d₆, 400 MHz) δ 7.04-7.06 (d, 1H), 6.94-7.01 (m, 1H), 6.50-6.52 (d, 1H), 6.06-6.11 (dd, 1H), 5.21-5.24 (dd, 1H), 4.92 (s, 2H), 3.75 (s, 3H).

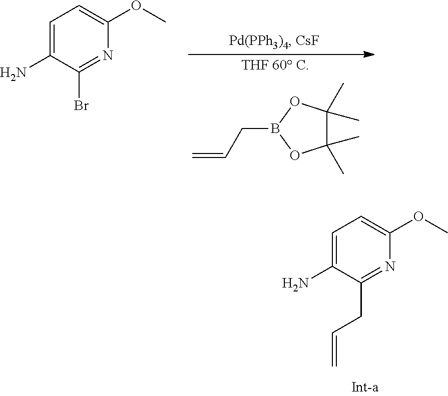

### Step A: 2-Allyl-6-methoxypyridin-3-amine Int-1a

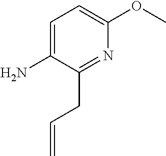

To a solution of 2-bromo-6-methoxypyridin-3-amine (5.0 g, 24.6 mmol) in THF (300 mL), purged with N₂, was added 2-allyl-4,4,5,5-tetramethyl-1,3,2-dioxaborolane (6.21 g, 36.9 mmol) followed by Pd(Ph₃P)₄ (1.99 g, 1.72 mmol) and CsF (14.9 g, 99.0 mmol) and the reaction mixture was sealed and heated at 62° C. for 0.5 h. Additional THF (100 mL) and H₂O (50 mL) were added to the reaction mixture and the reaction continued to heat at 62° C. for 5 h. The reaction mixture was allowed to cool to ambient temperature, diluted with EtOAc, washed with H₂O, brine, dried over Na₂SO₄, filtered and the solvent evaporated under reduced pressure. The crude residue was dissolved in DCM, preabsorbed onto a silica gel packed pre-column and purified by silica gel flash column chromatography (220 g) eluting with a 100% heptanes to 60% EtOAc-heptanes gradient. Product fractions were combined and evaporated under reduced pressure to afford 2-allyl-6-methoxypyridin-3-amine Int-1a (1.50 g, 37% yield) as a red oil. HPLC/MS 0.61 min (B), [M+H]⁺ 165.0. ¹H NMR (CDCl₃, 400 MHz) δ 3.38 (dt, J=6.36, 1.71 Hz, 2H), 3.79 (s, 3H), 5.02-5.08 (m, 2H), 5.89-6.01 (m, 1H), 5.92 (s, 1H), 6.41 (d, J=8.31 Hz, 1H), 6.87 (d, J=8.80 Hz, 1H)

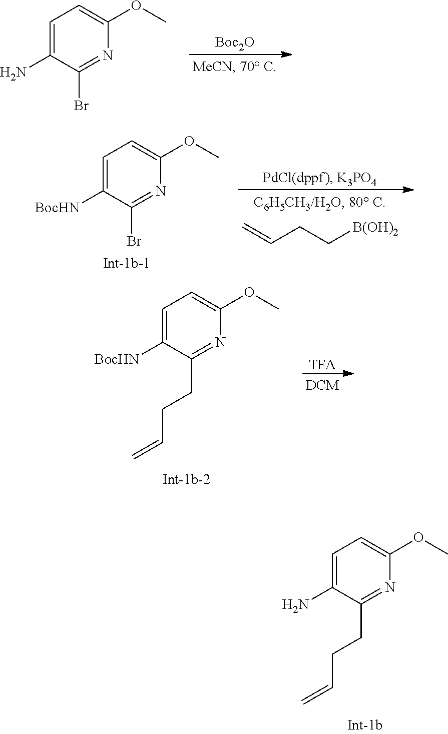

### Step A: tert-Butyl (2-bromo-6-methoxypyridin-3-yl)carbamate Int-1b-1

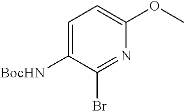 [SMILES: COc1ccc(NC(=O)OC(C)(C)C)c(Br)n1]

To a solution of 2-bromo-6-methoxypyridin-3-amine (5.05 g, 24.9 mmol) in MeCN (10 mL) was added boc-anhydride (23.1 mL, 99.0 mmol) and the reaction mixture stirred at 70° C. for 21 h. The reaction mixture was cooled to ambient temperature, loaded onto a silica gel packed pre-column and purified by silica gel flash column chromatography (330 g) eluting with a 100% heptanes to 20% heptanes-EtOAc gradient. Product fractions were combined and evaporated under reduced pressure to afford a yellow oil, which was further purified by silica gel flash column chromatography (330 g) eluting with a 50% heptanes-DCM to 100% DCM gradient. Product fractions were combined and evaporated under reduced pressure to afford tert-butyl (2-bromo-6-methoxypyridin-3-yl)carbamate Int-1b-1 (6.60 g, 74% yield) as a viscous yellow oil. HPLC/MS 1.07 min (A), [M+H]⁺ 305.0. ¹H NMR (DMSO-d₆, 400 MHz) δ 8.5-8.8 (m, 1H), 7.71 (d, 1H, J=8.8 Hz), 6.85 (d, 1H, J=8.3 Hz), 3.84 (s, 3H), 1.5-1.5 (m, 1H), 1.45 (s, 9H).

### Step B: tert-Butyl (2-(but-ene-1-yl)-6-methoxypyridin-3-yl)carbamate Int-1b-2

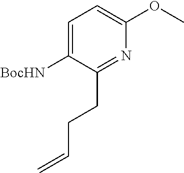 [SMILES: C=CCCc1nc(OC)ccc1NC(=O)OC(C)(C)C]

To tert-butyl (2-bromo-6-methoxypyridin-3-yl)carbamate (6.57 g, 21.7 mmol), but-3-en-1-ylboronic acid (4.33 g, 43.3 mmol), toluene (75 mL), and H₂O (15.00 mL), purged with N₂, were added PdCl(dppf)-CH₂Cl₂ adduct (1.77 g, 2.17 mmol) and K₃PO₄ (13.8 g, 65.0 mmol) and the reaction mixture was heated at 80° C. for 4 h. The reaction mixture was allowed to cool to ambient temperature, the reaction mixture diluted with EtOAc, washed with H₂O, brine, dried over Na₂SO₄, filtered and the solvent evaporated under reduced pressure. The crude residue was dissolved in DCM, loaded onto a silica gel packed pre-column and purified by silica gel chromatography (330 g) eluting with a 100% heptanes to 60% EtOAc-heptanes gradient. Product fractions were combined and evaporated under reduced pressure to afford tert-butyl (2-(but-ene-1-yl)-6-methoxypyridin-3-yl)carbamate Int-1b-2 (1.69 g, 24% yield) as a clear oil, in 82% purity. HPLC/MS 0.99 min (A), [M+H]⁺ 279.1. ¹H NMR (DMSO-d₆, 400 MHz) 8.3-8.8 (m, 1H), 7.3-7.6 (m, 1H), 6.5-6.7 (m, 1H), 5.89 (tdd, 1H, J=6.6, 10.3, 17.1 Hz), 5.4-5.7 (m, 1H), 4.9-5.1 (m, 2H), 3.8-3.8 (m, 4H), 2.71 (dd, 2H, J=6.6, 9.0 Hz), 2.3-2.5 (m, 2H), 1.6-1.6 (m, 1H), 1.44 (s, 9H).

### Step C: 2-(But-ene-1-yl)-6-methoxypyridin-3-amine Int-1b

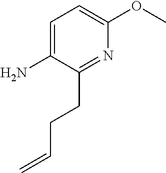

To a solution of tert-butyl (2-(but-3-en-1-yl)-6-methoxypyridin-3-yl)carbamate (338 mg, 1.21 mmol), in DCM (20 mL), was added TFA (0.936 mL, 12.1 mmol) and the reaction mixture was stirred at room temperature for 5 h. The reaction mixture was evaporated under reduced pressure, the residue partitioned and evaporated with CHCl₃ (3×) and dried under vacuo. The oil was dissolved in EtOAc, washed with sat'd aq. NaHCO₃, H₂O, brine, dried over Na₂SO₄, filtered and the solvent evaporated under reduced pressure. The residue was dissolved in DCM, absorbed onto a silica gel packed precolumn and purified by silica gel flash column chromatography (40 g) eluting with a 100% heptanes to 50% heptanes-EtOAc gradient. Product fractions were combined and evaporated under reduced pressure to afford 2-(but-ene-1-yl)-6-methoxypyridin-3-amine Int-1b (158 mg, 71% yield) as a light brown oil, in 93% purity. HPLC/MS 0.35 min (A), [M+H]⁺ 179.1. ¹H NMR (CD₃OD, 400 MHz) δ 7.12 (d, 1H, J=8.8 Hz), 6.5-6.5 (m, 1H), 5.5-5.7 (m, 1H), 4.9-5.1 (m, 2H), 3.8-3.8 (m, 4H), 2.7-2.8 (m, 2H), 2.48 (br dd, 2H, J=1.0, 7.8 Hz).

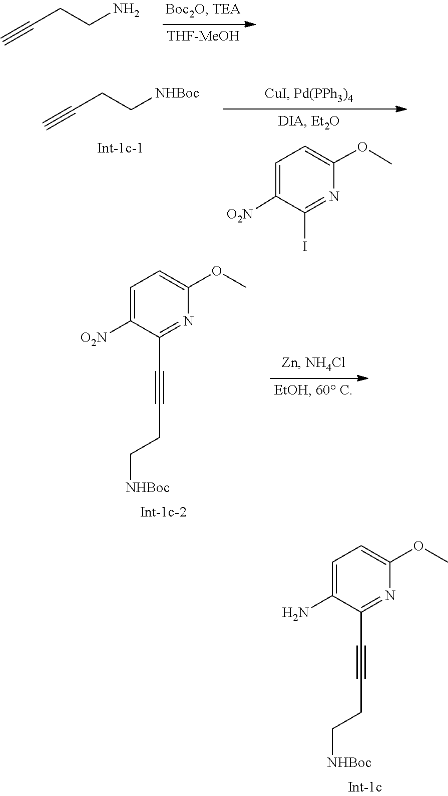

### Step A: tert-Butyl (but-3-yn-1-yl)carbamate Int-1c-1

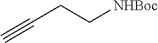

A stirred suspension of but-3-yn-1-amine, hydrochloride (4.26 g, 40.4 mmol) in MeOH (4 mL) was treated with TEA (11.8 mL, 85.0 mmol) and the reaction mixture stirred at room temperature until a clear solution. Boc-anhydride (9.25 g, 42.4 mmol), dissolved in THF (60 mL) was added in a slow stream and the reaction mixture was stirred at room temperature for 96 h. The solvent was evaporated under reduced pressure, the crude residue partitioned between DCM and H₂O, the layers separated, and the organic phase washed with brine, dried over Na₂SO₄, filtered and the solvent evaporated under reduced pressure to afford tert-butyl but-3-yn-1-ylcarbamate Int-1c-1 (6.57 g, 94% yield) as a colorless oil. ¹H NMR (CD₃CN, 400 MHz) δ 5.44 (br s, 1H), 3.18 (q, 2H, J=6.5 Hz), 2.34 (dt, 2H, J=2.4, 6.8 Hz), 2.22 (t, 1H, J=2.7 Hz), 1.4-1.4 (m, 9H).

### Step B: tert-Butyl (4-(6-methoxy-3-nitropyridin-2-yl)but-3-yn-1-yl)carbamate Int-1c-2

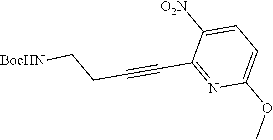 [SMILES: COc1ccc([N+](=O)[O-])c(C#CCCNC(=O)OC(C)(C)C)n1]

To a mixture of tert-butyl but-3-yn-1-ylcarbamate (0.500 g, 2.95 mmol) and 2-iodo-6-methoxy-3-nitropyridine (0.993 g, 3.55 mmol) in Et₂O (14.77 ml), was added diisopropylamine (2.07 ml, 14.8 mmol), copper(I) iodide (0.056 g, 0.295 mmol), and bis(triphenylphosphine)palladium(II) dichloride (0.104 g, 0.148 mmol) and the reaction mixture was stirred at room temperature for 70 min. The reaction mixture was poured into sat'd aq. NH₄Cl (300 mL), the layers separated, and the aqueous layer extracted with Et₂O (2×30 mL). The combined organic extracts were washed with brine, dried over Na₂SO₄, filtered and concentrated onto celite for purification. The reaction mixture was purified by silica gel flash column chromatography (40 g) eluting with a 100% heptanes to 20% EtOAc-heptanes gradient. Product fractions were combined and evaporated under reduced pressure to afford tert-butyl (4-(6-methoxy-3-nitropyridin-2-yl)but-3-yn-1-yl)carbamate Int-1c-2 (431 mg, 1.31 mmol, 45% yield) as a beige solid. HPLC/MS 1.05 min (A), [M-^{t}Bu]+266.1. ¹H NMR (CD₃CN, 400 MHz) δ 8.3-8.4 (m, 1H), 6.88 (d, 1H, J=8.8 Hz), 5.53 (br s, 1H), 4.00 (s, 3H), 3.33 (q, 2H, J=6.5 Hz), 2.72 (t, 2H, J=6.6 Hz), 1.44 (s, 9H).

### Step C: tert-Butyl (4-(3-amino-6-methoxypyridin-2-yl)but-3-yn-1-yl)carbamate Int-1c

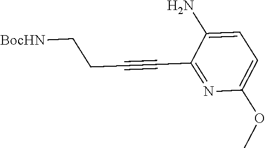 [SMILES: COc1ccc(N)c(C#CCCNC(=O)OC(C)(C)C)n1]

To a solution of tert-butyl (4-(6-methoxy-3-nitropyridin-2-yl)but-3-yn-1-yl)carbamate (107 mg, 0.333 mmol) in EtOH (3 mL) was added Zn (150 mg, 2.29 mmol) and NH₄Cl (107 mg, 1.99 mmol) and the reaction mixture was heated at 60° C. for 30 min. The reaction mixture was cooled to ambient temperature, the solids filtered, washed with EtOH, and the combined organics evaporated under reduced pressure. The residue was partitioned between EtOAc, H₂O, and sat'd aq. NaHCO₃, the layers separated, and the organic phase washed with brine, dried over Na₂SO₄, filtered and the solvent evaporated under reduced pressure to afford tert-butyl (4-(3-amino-6-methoxypyridin-2-yl)but-3-yn-1-yl)carbamate Int-1c (84 mg, 82% yield) as a yellow oil. HPLC/MS 0.82 min (A), [M+H]⁺ 292.1. ¹H NMR (CD₃CN, 400 MHz) δ 7.10 (d, 1H, J=8.8 Hz), 6.58 (d, 1H, J=8.8 Hz), 5.59 (br s, 1H), 4.33 (br s, 2H), 3.78 (s, 3H), 3.32 (q, 2H, J=6.4 Hz), 2.67 (t, 2H, J=6.6 Hz), 1.43 (s, 9H).

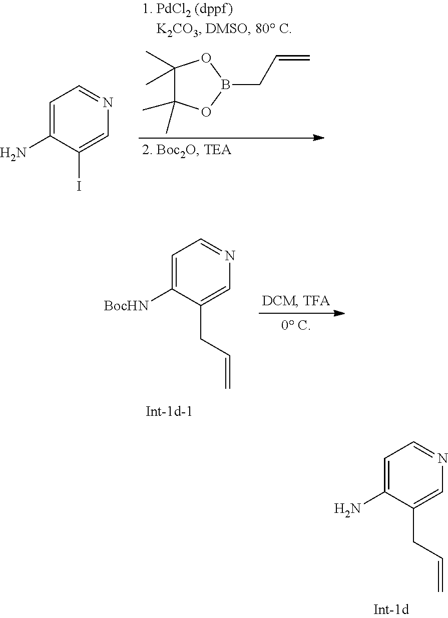

### Step A: tert-Butyl (3-allylpyridin-4-yl)carbamate Int-1d-1

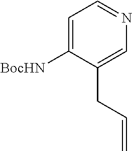 [SMILES: C=CCc1cnccc1NC(=O)OC(C)(C)C]

To a flask with a stir bar was added 3-iodopyridin-4-amine (2.00 g, 9.09 mmol), 2-allyl-4,4,5,5-tetramethyl-1,3,2-dioxaborolane (4.58 g, 27.3 mmol), and DMSO (36 mL), the reaction mixture purged with N₂, to which was added PdCl(dppf)-CH₂Cl₂ adduct (0.520 g, 0.636 mmol) and K₂CO₃ (3.14 g, 22.7 mmol) and the reaction mixture was heated at 80° C. for 4 h. The reaction mixture was cooled to ambient temperature, filtered over celite, the celite washed with EtOAc and the reaction mixture was concentrated under reduced pressure. The DMSO solution was diluted with DCM (˜3:1 DCM:DMSO) to which was added boc-anhydride (4.22 mL, 18.2 mmol) followed by TEA (5.07 mL, 36.4 mmol) and the reaction mixture was stirred at room temperature for 1 h. The reaction mixture was diluted with DCM, washed with H₂O (3×), dried over Na₂SO₄, filtered and the solvent evaporated under reduced pressure. The crude residue was dissolved in DCM, preabsorbed onto a silica gel precolumn and purified by silica gel flash column chromatography (80 g) eluting with a 100% heptanes to 100% 3:1 EtOAc-EtOH-heptanes gradient. Product fractions were combined and evaporated under reduced pressure to afford tert-butyl (3-allylpyridin-4-yl)carbamate Int-1d-1 (896 mg, 70% yield). HPLC/MS 0.93 min (B), [M+H]⁺ 235.1. ¹H NMR (DMSO-d₆, 400 MHz) δ 8.6-8.8 (m, 1H), 8.29 (s, 1H), 8.25 (s, 1H), 7.66 (d, 1H, J=5.9 Hz), 5.8-6.0 (m, 1H), 4.9-5.2 (m, 2H), 3.45 (d, 2H, J=6.4 Hz), 1.48 (s, 10H).

### Step B: 3-Allylpyridin-4-amine Int-1d

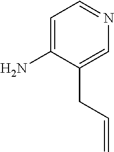

To tert-butyl (3-allylpyridin-4-yl)carbamate (896 mg, 3.82 mmol) in DCM (10 mL), cooled to 0° C., was added TFA (8.84 mL, 115 mmol) and the reaction mixture was stirred at 0° C. for 3 h. The reaction mixture was concentrated under reduced pressure and the residue azeotroped with CHCl₃ (3×) and dried under vacuo to afford 3-allylpyridin-4-amine Int-1d (1.24 g, 84% yield) as a dark, viscous oil. HPLC/MS 0.43 min (B), [M+H]⁺ 135.0. ¹H NMR (DMSO-d₆, 400 MHz) S 13.0-13.7 (m, 1H), 8.1-8.3 (m, 1H), 8.09 (br d, 1H, J=6.4 Hz), 8.03 (s, 1H), 7.2-7.7 (m, 1H), 6.84 (d, 1H, J=6.8 Hz), 5.7-6.1 (m, 1H), 4.9-5.3 (m, 2H), 3.28 (d, 2H, J=6.4 Hz).

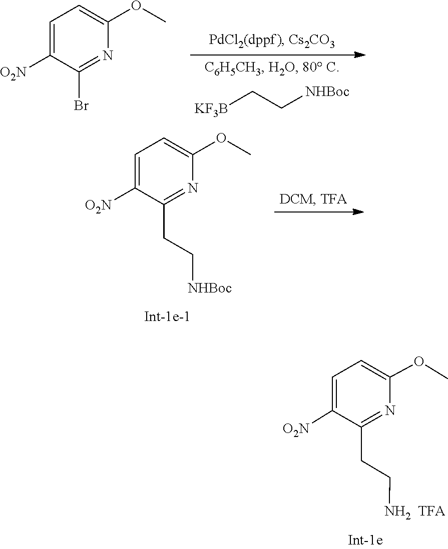

### Step A: tert-Butyl (2-(6-methoxy-3-nitropyridin-2yl)ethyl)carbamate Int-1e-1

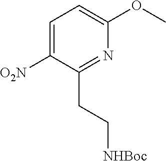 [SMILES: COc1ccc([N+](=O)[O-])c(CCNC(=O)OC(C)(C)C)n1]

To a mixture of 2-bromo-6-methoxy-3-nitropyridine (7.00 g, 30.0 mmol), toluene (130 mL) and H₂O (43.3 mL), purged with N₂, was added tert-butyl (2-(trifluoro-)₄-boraneyl)ethyl)carbamate, potassium salt (9.05 g, 36.0 mmol), PdCl₂(dppf)-CH₂Cl₂ adduct (0.491 g, 0.601 mmol) and Cs₂CO₃ (29.4 g, 90 mmol), and the reaction mixture was stirred under N₂ at 80° C. for 21 h. The reaction mixture was cooled to ambient temperature, diluted with EtOAc, washed with H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (330 g) eluting with a 100% heptane to 50% EtOAc gradient. Product fractions were combined and evaporated under reduced pressure to afford tert-butyl (2-(6-methoxy-3-nitropyridin-2yl)ethyl)carbamate Int-1e-1 (6.96 g, 76% yield) as a yellow solid. HPLC/MS 1.02 min (A), [M+H]⁺ 298.0. ¹H NMR (DMSO-d₆, 400 MHz) S 8.34 (d, 1H, J=9.3 Hz), 6.7-7.0 (m, 2H), 3.98 (s, 3H), 3.4-3.5 (m, 2H), 3.17 (s, 2H), 1.33 (s, 9H).

### Step B: 2-(6-Methoxy-3-nitropyridin-2yl)ethan-1-amine)carbamate, trifluoroacetate salt Int-1e

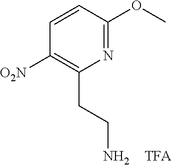

To a solution of tert-butyl (2-(6-methoxy-3-nitropyridin-2-yl)ethyl)carbamate (6.96 g, 23.4 mmol) in DCM (150 mL), at room temperature, was added TFA (54.1 mL, 702 mmol) and the reaction was stirred for 1 h. The reaction mixture was concentrated under reduced pressure, dissolved in CHCl₃ and evaporated under reduced pressure (repeated 3×) and dried under vacuo to afford 2-(6-methoxy-3-nitropyridin-2yl)ethan-1-amine)carbamate, TFA salt Int-1e (11.4 g, >100% yield) as a viscous oil. HPLC/MS 0.33 min (A), [M+H]⁺ 198.0. ¹H NMR (DMSO-d₆, 400 MHz) δ 8.43 (d, 1H, J=8.8 Hz), 8.32 (s, 1H), 7.86 (br s, 3H), 6.97 (d, 1H, J=9.3 Hz), 4.01 (s, 3H), 3.2-3.6 (m, 4H).

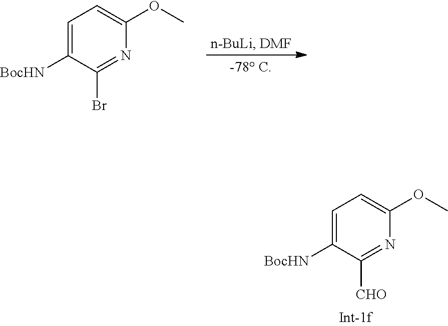

### Step A: tert-Butyl (2-formyl-6-methoxypyridin-3-yl)carbamate Int-1f

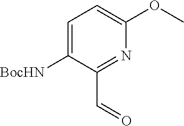 [SMILES: COc1ccc(NC(=O)OC(C)(C)C)c(C=O)n1]

A nitrogen-purged vessel containing tert-butyl (2-bromo-6-methoxypyridin-3-yl)carbamate (2.0 g, 6.60 mmol) and THF (33.0 ml) was cooled to −78° C. n-Butyllithium (6.60 ml, 16.49 mmol) was added drop-wise, and after 1 h DMF (3.07 ml, 39.6 mmol) was added in one portion. The reaction mixture was allowed to warm to ambient temperature and quenched with a sat'd aq. NH₄Cl solution. The organic layer was diluted with diethyl ether, washed with water, brine, and concentrated under reduced pressure. The crude material was added directly to a silica gel column and purified by silica gel flash column chromatography eluting with a heptanes/EtOAc gradient to afford tert-butyl (2-formyl-6-methoxypyridin-3-yl)carbamate Int-1f (800 mg, 48.1% yield) as a white solid. HPLC/MS 1.21 min (G), [M+H]⁺ 253.1. ¹H NMR (DMSO-d₆, 400 MHz): δ 9.96 (s, 1H), 9.88 (s, 1H), 8.57 (d, J=9.3 Hz, 1H), 7.20 (d, J=9.3 Hz, 1H), 3.92 (s, 3H), 1.50 (s, 9H).

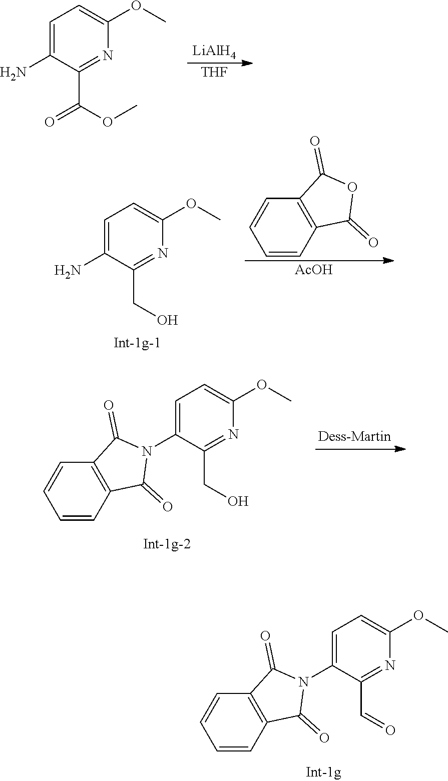

### Step A: (3-Amino-6-methoxypyridin-2-yl)methanol Int-1g-1

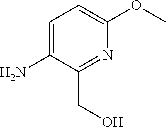

A solution of methyl 3-amino-6-methoxypicolinate (4.99 g, 27.4 mmol) in THF (100.00 mL) was cooled to 0° C., to which was slowly added a 2M LiAlH₄-THF sol'n (27.39 mL, 54.8 mmol). After 2 h the reaction mixture was quenched by a slow addition of sat'd aq. NH₄Cl (50 mL) and filtered. The filtrate was diluted with water (100 mL) and extracted with DCM (2×150 mL). The combined organics were washed with water (75 mL), dried over Na₂SO₄, filtered and concentrated under reduced pressure. The residue was dissolved in DMF and purified by reverse phase Combiflash (150 g Gold C₁₈ column) eluting with a 100% H₂O to 60% MeCN—H₂O (0.1% NH₄OH) gradient. Product fractions were combined and lyopholized to afford (3-amino-6-methoxypyridin-2-yl)methanol Int-1g-1 (2.11 g, 49% yield). HPLC/MS 0.37 min (B), [M+H]⁺ 155.0. ¹H NMR (400 MHz, CD₃OD) S 3.83 (s, 3H) 4.61 (s, 2H) 6.56 (d, J=8.80 Hz, 1H) 7.15 (d, J=8.80 Hz, 1H).

### Step B: 2-(2-(Hydroxymethyl)-6-methoxypyridin-3-yl)isoindoline-1,3-dione Int-12-2

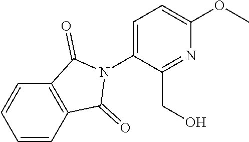 [SMILES: COc1ccc(N2C(=O)c3ccccc3C2=O)c(CO)n1]

To (3-amino-6-methoxypyridin-2-yl)methanol Int-1g-1 (1.06 6.89 mmol) in AcOH (20 mL) was added phthalic anhydride (1.02 g, 6.88 mmol). The mixture was heated to 100° C. for 1 h, concentrated, dissolved in DCM and washed with water. The aqueous phase was extracted with DCM, and the combined organic phases were dried, filtered and concentrated under reduced pressure. The crude residue was purified by Combiflash reverse phase chromatography (150 g C₁₈ Gold column, 40 min run, 0-60% MeCN in H₂O (both with 0.1% formic acid) eluting with a 100% H₂O (0.10% formic acid) to 60% MeCN/H₂O (0.10% formic acid) gradient. Product fractions were combined and lyopholyzed to afford 2-(2-(hydroxymethyl)-6-methoxypyridin-3-yl)isoindoline-1,3-dione Int-1g-2 (1.10 g, 56% yield) as a light brown solid. HPLC/MS 0.70 min (G), [M+H]⁺ 285.0. ¹H NMR (400 MHz, CD₃OD) S 4.00 (s, 3H) 4.52 (s, 2H) 6.84 (d, J=8.31 Hz, 1H) 7.61 (d, J=8.31 Hz, 1H) 7.86-7.90 (m, 2H) 7.93-7.98 (m, 2H).

### Step C: 3-(1,3-Dioxoisoindolin-2-yl)-6-methoxypicolinaldehyde Int-12

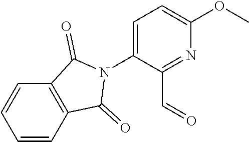 [SMILES: COc1ccc(N2C(=O)c3ccccc3C2=O)c(C=O)n1]

To a solution of 2-(2-(hydroxymethyl)-6-methoxypyridin-3-yl)isoindoline-1,3-dione Int-1g-2 (1.11 g, 3.90 mmol) in DCM (40 mL) at 0° C. was added Dess-Martin periodinane (2.32 g, 5.47 mmol) and the reaction mixture was warmed to RT and stirred for 2.5 h. 1 N NaOH (20 mL) was added, followed by water (30 mL) and DCM (30 mL). The mixture was stirred at RT for 15 min, the organic phase separated, the aqueous phase extracted with DCM (50 mL), and the combined organic solutions were washed with brine, dried over Na₂SO₄ and concentrated under reduced pressure. The crude residue was purified by Combiflash silica gel flash column chromatography (80 g), eluting with a 100% heptane to 60% EtOAc gradient. Product fractions were combined and evaporated under reduced pressure to afford 3-(1,3-dioxoisoindolin-2-yl)-6-methoxypicolinaldehyde Int-1g (1.03 g, 93% yield) as a white solid. HPLC/MS 0.88 min (A), [M+H]⁺ 283.1. H NMR (400 MHz, CDCl₃) δ 4.01-4.15 (m, 3H) 7.03-7.17 (m, 1H) 7.58-7.69 (m, 1H) 7.74-7.86 (m, 2H) 7.91-8.02 (m, 2H) 9.87-9.96 (m, 1H).

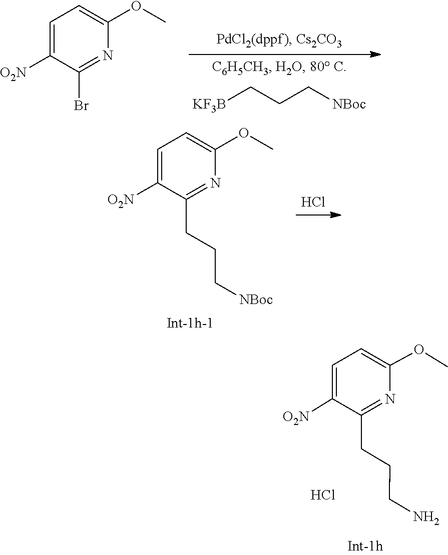

### Step A: tert-Butyl (2-(6-methoxy-3-nitropyridin-2yl)ethyl)carbamate Int-1h-1

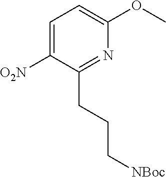

Following the procedure outlined in Int-1e-1, substituting tert-butyl (2-(trifluoro-)₄-boraneyl)ethyl)carbamate, potassium salt with tert-butyl (2-(trifluoro-)₄-boraneyl)propyl)carbamate, potassium salt, tert-butyl (3-(6-methoxy-3-nitropyridin-2-yl)propyl)carbamate Int-1h-1 (352 mg, 17% yield) was prepared as a yellow solid. HPLC/MS 1.08 min (A), [M-^{t}Bu]+256.1. ¹H NMR (CDCl₃, 400 MHz) δ 8.28 (d, 1H, J=8.8 Hz), 6.5-6.8 (m, 1H), 4.6-4.9 (m, 1H), 4.06 (s, 3H), 3.2-3.3 (m, 2H), 3.1-3.2 (m, 2H), 2.0-2.1 (m, 2H), 1.49 (s, 9H).

### Step B: 3-(6-Methoxy-3-nitropyridin-2-yl)propan-1-amine hydrochloride Int-1h

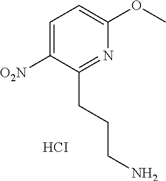

To tert-butyl(3-(6-methoxy-3-nitropyridin-2-yl)propyl)carbamate (703 mg, 2.26 mmol) in dichloromethane (5 mL) was added HCl (4M in dioxane) (16.9 mL, 67.7 mmol) and the reaction mixture was stirred for 2 h, diluted with Et₂O, and the resulting solid collected by filtration to afford 3-(6-methoxy-3-nitropyridin-2-yl)propan-1-amine hydrochloride Int-1h (507.8 mg, 91% yield) as a white solid. HPLC/MS 0.40 min (A), [M+H]⁺ 212.1. ¹H NMR (CD₃OD, 400 MHz) S 8.3-8.4 (m, 1H), 6.85 (d, 1H, J=8.8 Hz), 4.06 (s, 3H), 3.24 (s, 2H), 3.1-3.2 (m, 2H), 2.1-2.3 (m, 2H).

### Step A: 1-(6-Methoxy-3-nitropyridin-2-yl)propan-2-one Int-1i

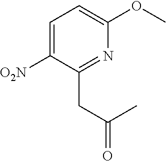

A mixture of 2-bromo-6-methoxy-3-nitropyridine (10.0 g, 42.9 mmol), 4-hydroxy-4 methylpentan-2-one (34.0 g, 292 mmol), PdOAc₂ (482 mg, 2.15 mmol), Ph₃P (2.25 g, 8.58 mmol) and Cs₂CO₃ (21.0 g, 64.3 mmol) in toluene (200 mL) was purged with N₂ for 3 minutes, heated at 110° C. for 4 h, cooled, filtered through a thin Celite pad (rinsing with EtOAc), concentrated and purified by silica gel flash column chromatography (330 g), eluting with 100% heptane to 15% EtOAc gradient. Product fractions were combined and evaporated under reduced pressure to afford 1-(6-methoxy-3-nitropyridin-2-yl)propan-2-one Int-l1 (3.95 g, 44% yield) as a yellow solid. HPLC/MS 0.71 min (B), [M+H]211.1. ¹H NMR (DMSO-d₆, 400 MHz): δ 8.45 (d, 1H, J=8.8 Hz), 6.98 (d, 1H, J=8.8 Hz), 4.34 (s, 2H), 3.96 (s, 3H), 2.26 (s, 3H).

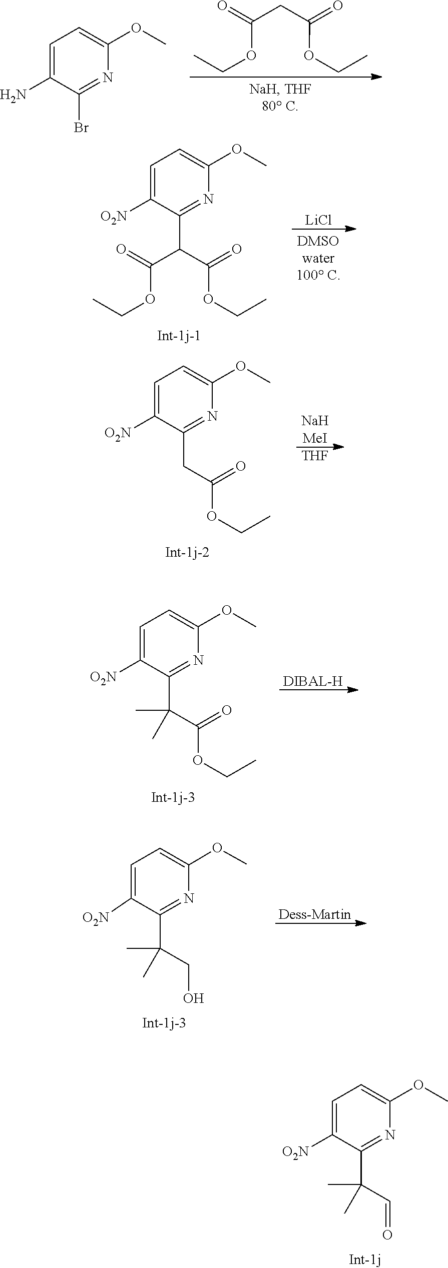

### Step A: Diethyl 2-(6-methoxy-3-nitropyridin-2-yl)malonate Int-l-1

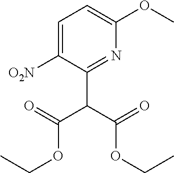 [SMILES: CCOC(=O)C(C(=O)OCC)c1nc(OC)ccc1[N+](=O)[O-]]

To a suspension of 60% NaH (2.54 g, 63.3 mmol) in THF (48 mL), cooled to 0° C. was added diethyl malonate (9.66 mL, 63.3 mmol), drop-wise. After stirring for 1 h, 2-chloro-6-methoxy-3-nitropyridine (6.00 g, 30.8 mmol) in THF (12 mL) was added. The reaction was stirred at 80° C. for 16 h, cooled to RT, quenched with cold H₂O (50 mL) and extracted with EtOAc (2×50 mL). The combined organics extracts were washed with H₂O (50 mL), brine (50 mL), dried over Na₂₂SO₄, filtered and concentrated under reduced pressure to afford crude diethyl 2-(6-methoxy-3-nitropyridin-2-yl)malonate Int-1j-1 (9.0 g, 84% yield) as a thick brown liquid, which was used without further purification. HPLC/MS 1.039 min (C), [M+H]⁺ 313.2.

### Step B: Ethyl 2-(6-methoxy-3-nitropyridin-2-yl)acetate Int-1j-2

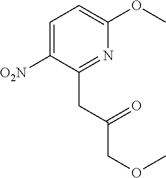 [SMILES: COCC(=O)Cc1nc(OC)ccc1[N+](=O)[O-]]

To a solution of diethyl 2-(6-methoxy-3-nitropyridin-2-yl)malonate (9.00 g, 28.8 mmol) in DMSO (50 mL) and H₂O (10 mL) was added LiCl (4.89 g, 115 mmol) and the reaction mixture was stirred at 100° C. for 16 h. The reaction was quenched with H₂O (50 mL) and extracted with EtOAc (2×50 mL). The combined organic extracts were washed with H₂O (50 mL), brine (50 mL), dried over Na₂₂SO₄, filtered, concentrated, dissolved in DCM (10 mL) and adsorbed onto silica gel (8 g). This slurry was purified by silica gel flash column chromatography (330 g), eluting with a 30% ethyl acetate-pet ether gradient. Product fractions were combined and evaporated under reduced pressure to afford ethyl 2-(6-methoxy-3-nitropyridin-2-yl)acetate Int-1j-2 (5.0 g, 68% yield) as colorless liquid. HPLC/MS 0.959 min (C), [M+H]⁺ 241.0.

### Step C: Ethyl 2-(6-methoxy-3-nitropyridin-2-yl)-2-methylpropanoate Int-1j-3

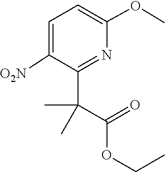 [SMILES: CCOC(=O)C(C)(C)c1nc(OC)ccc1[N+](=O)[O-]]

To ethyl 2-(6-methoxy-3-nitropyridin-2-yl)acetate Int-1j-2 (5.00 g, 20.8 mmol) in THF (50 mL) at 0° C. was added 60% NaH (2.50 g, 62.4 mmol). After 15 minutes, Mel (7.81 mL, 125 mmol) was added, and the reaction mixture was stirred at RT for 16 h, quenched with H₂O (50 mL) and extracted with EtOAc (2×50 mL). The combined organic extracts were washed with H₂O (50 mL), brine (50 mL), dried over Na₂SO₄, filtered and concentrated under reduced pressure to afford crude ethyl 2-(6-methoxy-3-nitropyridin-2-yl)-2-methylpropanoate Int-1j-3 (5.90 g, 100% yield) as a brown liquid, which was used without further purification. HPLC/MS 1.08 min (C), [M+H]⁺ 269.0.

### Step D: 2-(6-Methoxy-3-nitropyridin-2-yl)-2-methylpropan-1-ol Int-l1-4

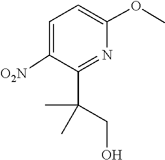 [SMILES: COc1ccc([N+](=O)[O-])c(C(C)(C)CO)n1]

A solution of ethyl 2-(6-methoxy-3-nitropyridin-2-yl)-2-methylpropanoate Int-1j-3 (4.00 g, 14.9 mmol) in Et₂O (40 mL) and DCM (8.00 mL) was cooled to −78° C., to which was added a 1M DIBAL-H in hexane sol'n (52.2 mL, 52.2 mmol). The reaction mixture was stirred at 0° C. for 3 h, quenched with H₂O (50 mL) and extracted with EtOAc (2×50 mL). The combined organic extracts were washed with H₂O (50 mL), brine (50 mL), dried over Na₂SO₄, filtered and concentrated under reduced pressure to afford crude 2-(6-methoxy-3-nitropyridin-2-yl)-2-methylpropan-1-ol Int-1j-4 (3.5 g, 76% yield), as a brown liquid, which was used without further purification. HPLC/MS 0.952 min (C), [M+H]⁺ 227.0.

### Step E: 2-(6-Methoxy-3-nitropyridin-2-yl)-2-methylpropanal Int-1j

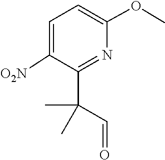 [SMILES: COc1ccc([N+](=O)[O-])c(C(C)(C)C=O)n1]

Following the procedure outlined in Int-1g, stirring at RT for 2 h and without purification, crude 2-(6-methoxy-3-nitropyridin-2-yl)-2-methylpropanal Int-1j (2.79 g, 63% yield) was obtained as brown liquid, which was used without further purification. HPLC/MS 1.109 min (C), [M+H]⁺ 225.0.

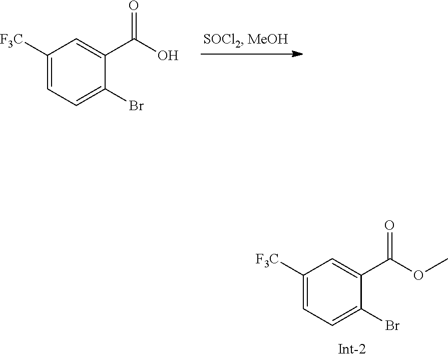

### Step A: Methyl 2-bromo-5-(trifluoromethyl)benzoate Int-2

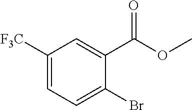

To a solution of 2-bromo-5-(trifluoromethyl)benzoic acid (20.0 g, 74.3 mmol) in MeOH (120 mL) was added SOCl₂ (7.93 mL, 149 mmol) at room temperature and the reaction mixture was stirred at 70° C. for 3 h. The reaction mixture cooled to 5° C., quenched with sat'd aq. NaHCO₃ until pH ˜8 and concentrated under reduced pressure. The aqueous phase was was extracted with EtOAc (100 mL), dried over Na₂SO₄, filtered and evaporated under reduced pressure to afford methyl 2-bromo-5-(trifluoromethyl)benzoate Int-2 (19.0 g, 90% yield) as a clear oil. HPLC/MS 3.19 min (H). ¹H NMR (400 MHz, DMSO-d₆) δ 8.11 (d, J=2.0 Hz, 1H), 8.02 (d, J=8.4 Hz, 1H), 7.86 (dd, J=1.6 Hz, 8.4 Hz, 1H), 3.90 (s, 3H).

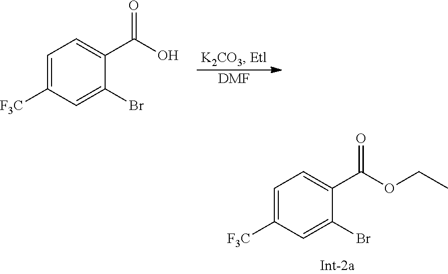

### Step A: Ethyl 2-bromo-4-(trifluoromethyl)benzoate Int-2a

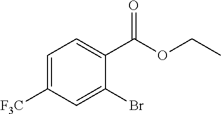 [SMILES: CCOC(=O)c1ccc(C(F)(F)F)cc1Br]

To a solution of 2-bromo-4-(trifluoromethyl)benzoic acid (35.0 g, 130 mmol) in DMF (250 mL), under N₂ and cooled to 0° C., was added K₂CO₃ (25.2 g, 182 mmol), portion-wise over 10 min. The reaction mixture was stirred at room temperature for 10 min, to which was added ethyl iodide (12.6 mL, 156 mmol), drop-wise, and the reaction mixture stirred for 4 h. The reaction mixture was quenched with ice-cold water (500 mL), extracted with EtOAc (3×200 mL), the combined organic extracts washed with H₂O (3×500 mL), brine, dried over Na₂SO₄, filtered and evaporated under reduced pressure to afford ethyl 2-bromo-4-(trifluoromethyl)benzoate Int-2a (38.0 g, 98% yield) as a yellow oil. GC/MS 3.13 min, [M+H]⁺ 295.9. ¹H NMR (400 MHz, DMSO-d₆) S 8.15 (s, 1H), 7.88-7.94 (m, 1H), 4.34-4.40 (q, 2H), 1.32-1.35 (t, 3H).

### Step A: Methyl 2-chloro-5-(trifluoromethyl)nicotinate Int-2b

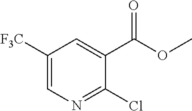

Following the procedure for Int-2a, substituting iodomethane for ethyl iodide and 2-chloro-5-(trifluoromethyl)nicotinic acid for 2-bromo-4-(trifluoromethyl)benzoic acid, methyl 2-chloro-5-(trifluoromethyl)nicotinate Int-2b (15.0 g, 94% yield) was prepared as a brown oil. HPLC/MS 0.65 min (C), [M+H]⁺ 240.0. ¹H NMR (DMSO-d₆, 400 MHz) δ 9.06 (s, 1H), 8.65 (s, 1H), 3.92 (s, 3H).

### Step A: Methyl 2-bromo-4-(trifluoromethyl)benzoate Int-2c

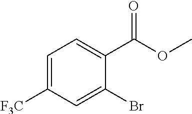

Following the preparation for Int-2, substituting 2-bromo-5-(trifluoromethyl)benzoic acid with 2-bromo-4-(trifluoromethyl)benzoic acid and heating the reaction mixture at 80° C. for 12 h, methyl 2-bromo-4-(trifluoromethyl)benzoate Int-2c (10.2 g, 83% yield) was isolated as yellow oil. HPLC/MS 1.11 min (A), [M+H]⁺ did not ionize. ¹H NMR (DMSO-d₆, 400 MHz) δ 8.16 (s, 1H), 7.9-8.0 (m, 1H), 7.9-7.9 (m, 1H), 3.91 (s, 3H)

### Methyl 2-bromo-4-(trifluoromethoxy)benzoate Int-2d

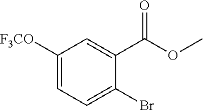 [SMILES: COC(=O)c1cc(OC(F)(F)F)ccc1Br]

Following the preparation for Int-2a, substituting 2-bromo-5-(trifluoromethyl)benzoic acid with 2-bromo-4-(trifluoromethoxy)benzoic acid, and stirring the reaction mixture at room temperature for 1 h, methyl 2-bromo-4-(trifluoromethoxy)benzoate Int-2d (4.56 g, 83% yield) was isolated as clear oil. HPLC/MS 1.09 min (A), [M+H]⁺ 298.9. ¹H NMR (DMSO-d₆, 400 MHz) δ 7.91 (d, 1H, J=8.8 Hz), 7.7-7.8 (m, 1H), 7.55 (ddd, 1H, J=1.0, 2.9, 8.8 Hz), 3.89 (s, 3H).

### Step A: Methyl 2-bromo-5-fluoro-4-(trifluoromethyl)benzoate Int-2e

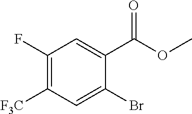 [SMILES: COC(=O)c1cc(F)c(C(F)(F)F)cc1Br]

To a solution of 2-bromo-5-fluoro-4-(trifluoromethyl)benzoic acid (10.0 g, 34.8 mmol) and MeOH (14.1 ml, 348 mmol) was added H₂SO₄ (0.371 ml, 6.97 mmol) and the reaction mixture was stirred at 90° C. for 72 h. The reaction mixture was cooled to ambient temperature and the solvent was evaporated under reduced pressure. The solution was partitoned with EtOAc and sat'd aq. NaHCO₃, the layers were separated, and the aqueous layer was extracted with EtOAc. The combined organic layers were washed with brine, dried over MgSO₄, filtered, and the solvent evaporated under reduced pressure to afford methyl 2-bromo-5-fluoro-4-(trifluoromethyl)benzoate Int-2e (9.82 g, 94% yield) as a clear oil. HPLC/MS 1.18 min (B), [M+H]⁺ did not ionize. ¹H NMR (CDCl₃, 400 MHz) δ 7.89 (d, J=6.36 Hz, 1H), 7.63 (d, J=9.78 Hz, 1H), 3.98 (s, 3H).

### Step A: Methyl 6-bromo-2-fluoro-3-(trifluoromethyl)benzoate Int-2f

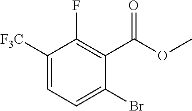 [SMILES: COC(=O)c1c(Br)ccc(C(F)(F)F)c1F]

Following the preparation for Int-2e, substituting 2-bromo-5-fluoro-4-(trifluoromethyl)benzoic acid with 6-bromo-2-fluoro-3-(trifluoromethyl)benzoic acid, methyl 6-bromo-2-fluoro-3-(trifluoromethyl)benzoate Int-2f (6.59 g, 62% yield) was isolated as yellow oil. HPLC/MS 1.15 min (B), [M+H]⁺ did not ionize. ¹H NMR (CDCl₃, 400 MHz) δ 7.47-7.55 (m, 2H), 3.99 (s, 3H).

### Step A: Methyl 6-amino-2-fluoro-3-(trifluoromethyl)benzoate Int-2g

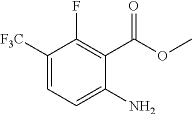 [SMILES: COC(=O)c1c(N)ccc(C(F)(F)F)c1F]

Methyl 6-bromo-2-fluoro-3-(trifluoromethyl)benzoate Int-2f (2.0 g, 6.64 mmol), copper metal (0.422 g, 6.64 mmol), TMS-N₃ (1.76 ml, 13.3 mmol) and 2-aminoethane-1-ol (1.00 ml, 16.6 mmol) in DMA (15 ml) were heated to 95° C. for 4 h, diluted with ethyl acetate and H₂O and filtered through a Celite pad. The layers were separated and the aqueous phase was extracted with EtOAc. The combined organic extracts were washed with brine, dried over MgSO₄, filtered, and concentrated under reduced pressure. The crude residue was dissolved in DCM (5 mL), adsorbed on a silica gel pre-column, dry load injected on a Teledyne-Isco RediSep Rf silica gel gold column (80 g) and eluted with a 100% heptanes to 100% EtOAc gradient. Product fractions were combined and evaporated under reduced pressure to afford methyl 6-amino-2-fluoro-3-(trifluoromethyl)benzoate Int-2g (422 mg, 26% yield) as a white solid. HPLC/MS 1.01 min (B), [M+H]⁺ 237.8. ¹H NMR (CDCl₃, 400 MHz) δ 7.26 (s, 1H), 6.31 (d, J=9.29 Hz, 1H), 5.95 (br s, 2H), 3.79 (s, 3H).

### Step A: Methyl 2-bromo-5-(2,2,2-trifluoroethoxy)benzoate Int-2h

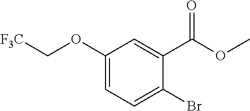 [SMILES: COC(=O)c1cc(OCC(F)(F)F)ccc1Br]

A mixture of methyl 2-bromo-5-hydroxybenzoate (3.50 g, 15.2 mmol), 2,2,2-trifluoroethyl 4-methylbenzenesulfonate (4.24 g, 16.7 mmol) and Cs₂CO₃ (5.43 g, 16.7 mmol) in DMF (50 mL) was stirred under nitrogen at 90° C. for 20 h, diluted with ethyl acetate, washed with water (2×), brine, dried over Na₂SO₄, filtered, and concentrated under reduced pressure. The crude residue was purified by silica gel flash column chromatography (220 g), eluting with a 10% EtOAc-heptanes to 20% EtOAc-heptane gradient. Product fractions were combined and evaporated under reduced pressure to afford methyl 2-bromo-5-(2,2,2-trifluoroethoxy)benzoate Int-2h (3.30 g, 70% yield). HPLC/MS 1.13 min (A), [M+H]⁺ 313. ¹H NMR (CDCl₃, 400 MHz): δ 7.62 (d, J=8.8 Hz, 1H), 7.40 (d, J=2.9 Hz, 1H), 6.95-7.01 (m, 1H), 4.39 (q, J=8.2 Hz, 2H), 3.97 (s, 3H).

### Step A: Methyl 2-amino-5-chloro-4-(trifluoromethyl)benzoate Int-2i

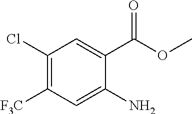 [SMILES: COC(=O)c1cc(Cl)c(C(F)(F)F)cc1N]

A mixture of methyl 2-amino-5-iodo-4-(trifluoromethyl)benzoate (2.40 g, 6.96 mmol) and copper(I) chloride (2.40 g, 24.3 mmol) in NMP (6.00 mL) was purged with N₂, stirred at 150° C. for 1 h, cooled to ambient temperature, quenched with 1N HCl and extracted with EtOAc (3×).

The combined organic extracts were washed with brine, dried over Na₂SO₄, filtered, and concentrated under reduced pressure. The crude residue wsa dissolved in DCM, preabsorbed onto a silica gel packed pre-column and purified by normal phase silica gel flash chromatography, eluting with a 100% heptane to 60% EtOAc-heptane gradient. Product fractions were combined and evaporated under reduced pressure to afford methyl 2-amino-5-chloro-4-(trifluoromethyl)-benzoate Int-2i (1.26 g, 68% yield) as a light yellow solid. HPLC/MS 1.13 min (A), [M+H]⁺ 254.0. ¹H NMR (CDCl₃, 400 MHz) δ 8.00 (s, 1H), 7.04 (s, 1H), 5.8-6.1 (m, 2H), 3.9-4.0 (m, 3H).

### Step A: Methyl 2-amino-5-fluoro-4-(trifluoromethyl)benzoate Int-2j

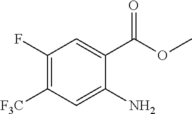 [SMILES: COC(=O)c1cc(F)c(C(F)(F)F)cc1N]

A suspension of 2-bromo-4-fluoro-5-(trifluoromethyl)aniline (40 g, 155 mmol) in MeOH (400 mL) was added PdCl₂(dppf)-CH₂Cl₂adduct (12.7 g, 15.5 mmol) under N₂ atmosphere, was degassed and purged with CO (3×) and the reaction mixture was stirred under CO (0.5 MPa) at 80° C. for 12 h. The reaction mixture was cooled to ambient temperature, filtered through celite, and the filtrate was concentrated under reduced pressure. The residue was diluted with MTBE (300 mL), the organic layer washed with sat'd NH₄Cl (300 mL), dried over Na₂SO₄, filtered and concentrated under reduced pressure. The crude product (67 g) was dissolved in DCM (200 mL), and 80 g of silica gel (100-200 mesh silica gel) was added. The resulting mixture was concentrated at 45° C. to give a dry flowing solid which was loaded into 180 g of silica gel (self-prepared column chromatography, 100-200 mesh silica gel) and eluted with a 80:1 n-heptane/EtOAc to a 4:1 n-heptane/EtOAc gradient. The desired product fraction were combined and evaporated under reduced pressure to afford methyl 2-amino-5-fluoro-4-(trifluoromethyl)benzoate Int-2j (41 g, >100% yield) as a white solid. HPLC/MS 2.69 min (I), [M+H]⁺ 238.1. ¹H NMR (DMSO-d₆, 400 MHz) δ 7.67-7.64 (d, 1H), 7.19-7.21 (m, 1H), 6.85 (s, 2H), 3.82 (s, 3H).

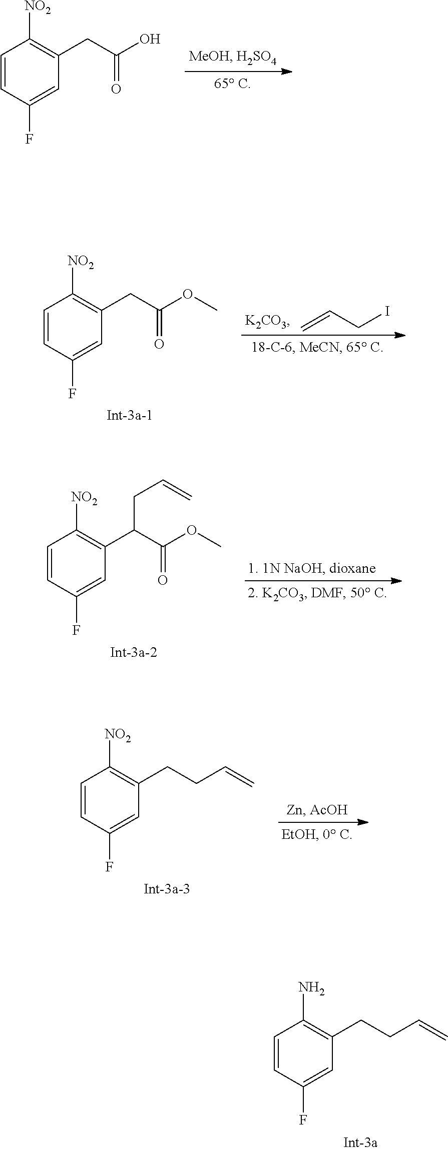

### Step A: Methyl 2-(5-fluoro-2-nitrophenyl)acetate Int-3a-1

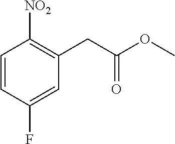

To a solution of 2-(5-fluoro-2-nitrophenyl)acetic acid (10.3 g, 51.9 mmol), in MeOH (50 mL), was added H₂SO₄ (0.332 mL, 6.22 mmol), dropwise, and the reaction mixture was stirred at 65° C. for 21 h. The reaction mixture was cooled to ambient temperature, the solvent evaporated under reduced pressure, the residue dissolved in EtOAc, dried over Na₂SO₄, filtered and the solvent evaporated under reduced pressure. The crude residue was dissolved in DCM, absorbed onto a silica gel packed precolumn and purified by silica gel flash column chromatography (330 g) eluting with a 100% heptane to 60% EtOAc gradient. Product fractions were combined and evaporated under reduced pressure to afford methyl 2-(5-fluoro-2-nitrophenyl)acetate Int-3a-1 (10.8 g, 91% yield) as a yellow oil. HPLC/MS 0.75 min (A); ¹H NMR (DMSO-d₆, 400 MHz) δ 8.24 (dd, 1H, J=5.1, 9.0 Hz), 7.52 (d, 1H, J=9.3 Hz), 7.4-7.5 (m, 1H), 4.12 (s, 2H), 3.63 (s, 3H).

### Step B: Methyl 2-(5-fluoro-2-nitrophenyl)pent-4-enoate Int-3a-2

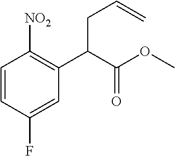 [SMILES: C=CCC(C(=O)OC)c1cc(F)ccc1[N+](=O)[O-]]

To a solution of methyl 2-(5-fluoro-2-nitrophenyl)acetate (7.92 g, 37.2 mmol), in MeCN (170 mL), was added K₂CO₃ (43.1 g, 312 mmol), followed by 18-crown-6 (0.098 g, 0.372 mmol).

To the purple reaction mixture was then added 3-iodoprop-1-ene (4.08 mL, 44.6 mmol), dropwise at room temperature, and the reaction mixture was stirred at room temperature for 22 h, then at 75° C. for 21 h. The reaction mixture was diluted with EtOAc, washed with H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (330 g) eluting with a 100% heptane to 50% EtOAc gradient. Product fractions were combined and evaporated under reduced pressure to afford methyl 2-(5-fluoro-2-nitrophenyl)pent-4-enoate Int-3a-2 (8.02 g, 83% yield) as a clear, light yellow oil. HPLC/MS 1.01 min (A), [M+H]⁺ 254.0. ¹H NMR (DMSO-d₆, 400 MHz) δ 8.12 (dd, 1H, J=5.1, 9.0 Hz), 7.3-7.6 (m, 2H), 5.5-5.8 (m, 1H), 4.8-5.1 (m, 2H), 4.31 (dd, 1H, J=6.4, 8.8 Hz), 3.61 (s, 3H), 2.8-2.9 (m, 1H), 2.6-2.7 (m, 1H).

### Step C: 2-(But-3-en-1-yl)-4-fluoro-1-nitrobenzene Int-3a-3

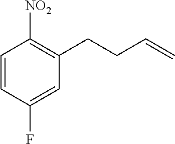

To a solution of methyl 2-(5-fluoro-2-nitrophenyl)pent-4-enoate (16.9 g, 66.7 mmol), in 1,4-dioxane (350 mL), was added 1M NaOH (80 mL, 80 mmol), and the reaction mixture was stirred at room temperature for 24 h. The solvent was evaporated under reduced pressure and the residue was dissolved in H₂O, acidified with 6M HCl, extracted with EtOAc, the organic phase washed with H₂O, brine, dried over Na₂SO₄, filtered and the solvent evaporated under reduced pressure to afford a light yellow solid. The solid was dissolved in DMF (200 mL) to which was added K₂CO₃ (46.1 g, 334 mmol) and the reaction mixture stirred at 50° C. for 3 h. The reaction mixture was cooled, diluted with EtOAc, washed with H₂O (3×), brine, dried over Na₂SO₄, filtered and the solvent evaporated under reduced pressure to afford 2-(but-3-en-1-yl)-4-fluoro-1-nitrobenzene Int-3a-3 (12.7 g, 88% yield) as a light brown oil. HPLC/MS 1.10 min (A). ¹H NMR (DMSO-d₆, 400 MHz) δ 8.07 (dd, 1H, J=5.1, 9.0 Hz), 7.43 (dd, 1H, J=2.9, 9.8 Hz), 7.33 (ddd, 1H, J=2.9, 7.8, 8.8 Hz), 5.84 (dd, 1H, J=10.3, 17.1 Hz), 4.8-5.1 (m, 2H), 2.9-3.0 (m, 2H), 2.3-2.4 (m, 2H).

### Step D: 2-(But-3-en-1-yl)-4-fluoroaniline Int-3a

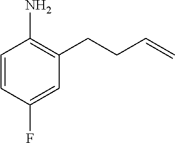

To a suspension of 2-(but-3-en-1-yl)-4-fluoro-1-nitrobenzene (12.7 g, 65.1 mmol), in EtOH (300 ml) was added zinc (63.8 g, 976 mmol), the suspension cooled to 0° C. in a salt-ice bath, to which was added acetic acid (48.4 ml, 846 mmol), slowly and dropwise at 0° C. for 1.5 h. The reaction mixture was filtered, the filtrate washed with EtOH, the ethanol removed under reduced pressure, the solid partitioned between EtOAc and 10% NaHCO₃, the layers separated, and the organic phase washed with H₂O, brine, dried over Na₂SO₄, filtered and the solvent evaporated under reduced pressure to afford a brown oil. The oil was dissolved in DCM, preabsorbed onto a silica gel packed pre-column and purified by silica gel flash column chromatography (120 g) eluting with a 10% EtOAc-hexanes to 100% EtOAc gradient. Product fractions were combined and evaporated under reduced pressure to afford 2-(but-3-en-1-yl)-4-fluoroaniline Int-3a (8.47 g, 71% yield) as a brown oil. HPLC/MS 0.49 min (A), [M+H]⁺ 166.0. ¹H NMR (DMSO-d₆, 400 MHz) δ 6.7-6.8 (m, 2H), 6.59 (dd, 1H, J=5.4, 8.8 Hz), 5.87 (tdd, 1H, J=6.6, 10.3, 17.1 Hz), 4.9-5.2 (m, 2H), 4.72 (s, 2H), 2.1-2.4 (m, 2H).

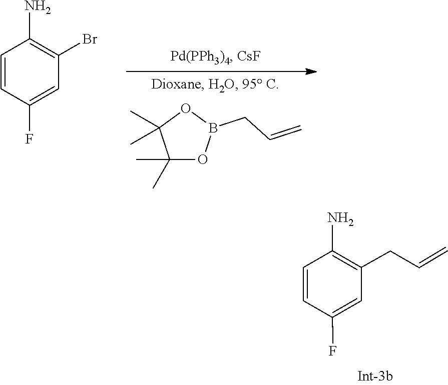

### Step A: 2-Allyl-4-fluoroaniline Int-3b

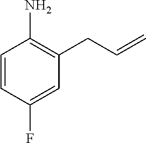

To a solution of 2-bromo-4-fluoroaniline (5.06 g, 26.6 mmol) in 1,4-dioxane (100 mL) and H₂O (10.0 mL), under N₂, was added 2-allyl-4,4,5,5-tetramethyl-1,3,2-dioxaborolane (7.49 mL, 39.9 mmol), followed by Pd(PPh₃)₄ (3.08 g, 2.66 mmol) and CsF (16.2 g, 107 mmol) and the reaction mixture was stirred at 95° C. for 18 h. The reaction mixture was allowed to cool to ambient temperature, concentrated under vacuum and diluted with EtOAc (250 mL). The resulting mixture was washed with H₂O, brine, dried over MgSO₄, filtered, and the solvent evaporated under reduced pressure. The crude residue was purified by silica gel flash column chromatography (120 g) eluting with a 100% heptanes to 25% EtOAc-heptanes gradient. Product fractions were combined and evaporated under reduced pressure to afford 2-allyl-4-fluoroaniline Int-3b (1.12 g, 26% yield) as a brown oil. HPLC/MS 0.83 min (B), [M+H]⁺ 152.0. ¹H NMR (DMSO-d₆, 400 MHz) δ 6.7-6.8 (m, 2H), 6.61 (dd, 1H, J=5.4, 8.8 Hz), 5.93 (tdd, 1H, J=6.7, 10.2, 16.9 Hz), 5.0-5.2 (m, 2H), 4.70 (s, 2H), 3.19 (d, 2H, J=6.8 Hz).

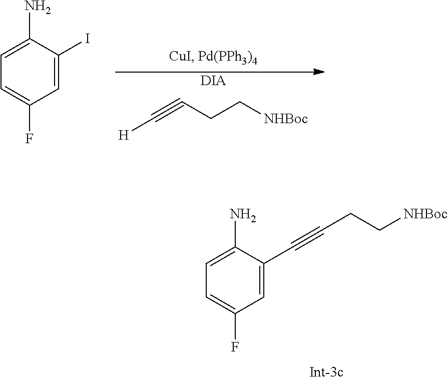

### Step A: tert-Butyl (4-(2-amino-5-fluorophenyl)but-3-yn-1-yl)carbamate Int-3c

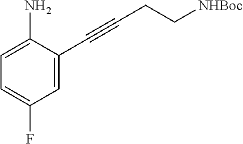 [SMILES: CC(C)(C)OC(=O)NCCC#Cc1cc(F)ccc1N]

To a stirred solution of tert-butyl but-3-yn-1-ylcarbamate (1.00 g, 5.91 mmol) and 4-fluoro-2-iodoaniline (1.54 g, 6.50 mmol) in diisopropylamine (20 mL) was added copper(I) iodide (0.113 g, 0.591 mmol) and Pd(Ph₃)₄ (0.082 g, 0.071 mmol) under N₂ atmosphere. The reaction mixture was stirred at room temperature for 16 h, diluted with H₂O (100 mL) and extracted with EtOAc (2×50 mL). The combined organic extracts were washed with brine (50 mL), dried over Na₂SO₄, filtered, and the solvent evaporated under reduced pressure. The crude product was dissolved in DCM, adsorbed on silica gel and purified by silica gel column chromatography (40 g) eluting with 20% of ethyl acetate-pet. ether. Product fractions were combined and evaporated under reduced pressure to afford tert-butyl (4-(2-amino-5-fluorophenyl)but-3-yn-1-yl)carbamate Int-3c (1.48 g, 82% yield) as brown gum. HPLC/MS 1.45 min (C), [M+H]⁺ 279.2. ¹H NMR (DMSO-d₆, 400 MHz) δ 7.06-7.09 (m, 1H), 6.88-6.92 (m, 2H), 6.85-6.86 (d, 1H), 5.19 (s, 2H), 3.14-3.19 (m, 2H), 2.51-2.56 (m, 2H), 1.38 (s, 9H).

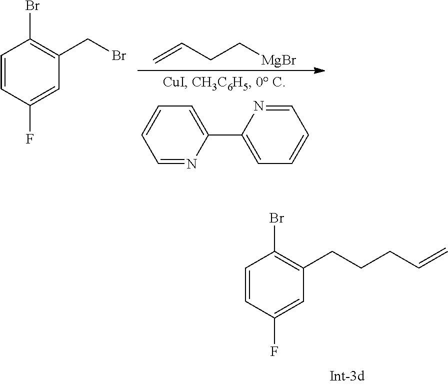

### Step A: 1-Bromo-4-fluoro-2-(pent-4-en-1-yl)benzene Int-3d

To a solution of 1-bromo-2-(bromomethyl)-4-fluorobenzene (25.0 g, 93.0 mmol) in toluene (250 mL), under N₂ at 0° C., was added copper(I) iodide (1.78 g, 9.33 mmol) and 2,2′-bipyridine (1.46 g, 9.33 mmol), followed by a 0.5M but-3-en-1-ylmagnesium bromide in THF solution (560 mL, 280 mmol), and the reaction mixture was stirred at room temperature for 2 h. The reaction mixture was quenched with sat'd aq. NH₄Cl (250 mL) and extracted with EtOAc (2×100 mL). The combined organic extracts were washed with H₂O (200 mL), brine, dried over Na₂SO₄, filtered, and the solvent evaporated under reduced pressure. The crude residue was dissolved in DCM, adsorbed on silica gel and purified by silica gel column chromatography (120 g) eluting with pet. ether. Product fractions were combined and evaporated under reduced pressure to afford 1-bromo-4-fluoro-2-(pent-4-en-1-yl)benzene Int-3d (9.0 g, 36% yield) as a clear liquid. GC/MS 3.48 min, [M+H]⁺ 242.0. ¹H NMR (DMSO-d₆, 400 MHz) δ 7.59-7.62 (m, 1H), 7.22-7.25 (m, 1H), 7.01-7.06 (m, 1H), 5.79-5.89 (m, 1H), 4.97-5.07 (m, 2H), 2.66-2.70 (m, 2H), 2.06-2.12 (m, 2H), 1.61-1.68 (m, 2H).

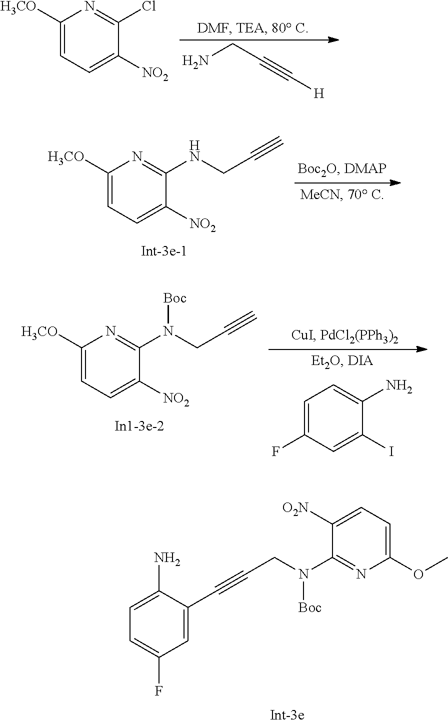

### Step A: 6-Methoxy-3-nitro-N-(prop-2-yn-1-yl)pyridin-2-amine Int-3e-1

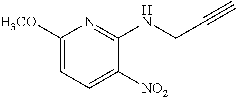

To a solution of 2-chloro-6-methoxy-3-nitropyridine (5.60 g, 29.7 mmol) in DMF (112 ml), was added prop-2-yn-1-amine (1.90 ml, 29.7 mmol) and TEA (8.28 ml, 59.4 mmol), and the reaction mixture was stirred at 80° C. for 4 h. Additional prop-2-yn-1-amine (0.476 ml, 7.42 mmol) was added and heated for 6 h, then stirred at room temperature for an additional 12 h. The reaction mixture was poured into ice (250 mL) and filtered to afford 6-methoxy-3-nitro-N-(prop-2-yn-1-yl)pyridin-2-amine Int-3e-1 (5.5 g, 88% yield) as a light brown solid. HPLC/MS 0.87 min (A), [M+H]⁺ 208.0. ¹H NMR (DMSO-d₆, 400 MHz) δ 8.8-9.1 (m, 1H), 8.2-8.4 (m, 1H), 6.1-6.3 (m, 1H), 4.32 (d, 2H, J=2.4 Hz), 3.99 (s, 4H), 3.10 (s, 1H).

### Step B: tert-Butyl (6-methoxy-3-nitropyridin-2-yl)(prop-2-yn-1-yl)carbamate Int-3e-2

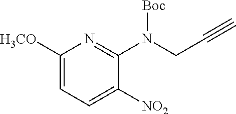 [SMILES: C#CCN(C(=O)OC(C)(C)C)c1nc(OC)ccc1[N+](=O)[O-]]

To a solution of 6-methoxy-3-nitro-N-(prop-2-yn-1-yl)pyridin-2-amine (5.40 g, 26.1 mmol), in MeCN (111 ml), was added boc-anhydride (12.1 ml, 52.1 mmol) and DMAP (0.637 g, 5.21 mmol), and the reaction mixture was stirred at 70° C. for 30 min, then 18 h at ambient temperature. The solvent was evaporated under reduced pressure to an oil, which was triturated with EtOAc (50 mL), filtered, and the filtrate preabsorbed onto silica and purified by silica gel flash column chromatography (40 g) eluting with a 100% heptane to 50% EtOAc gradient. Product fractions were combined and evaporated under reduced pressure to afford tert-butyl (6-methoxy-3-nitropyridin-2-yl)(prop-2-yn-1-yl)carbamate Int-3e-2 (7.38 g, 92% yield) as a yellow oil. HPLC/MS 1.12 min (A), [M-Boc]⁺ 252.0. ¹H NMR (DMSO-d₆, 400 MHz) δ 8.38 (d, 1H, J=8.8 Hz), 6.87 (d, 1H, J=8.8 Hz), 4.65 (br s, 2H), 3.99 (s, 3H), 3.1-3.2 (m, 1H), 1.36 (br s, 9H).

### Step C: tert-Butyl (3-(2-amino-5-fluorophenyl)prop-2-yn-1-yl)(6-methoxy-3-nitropyridin-2-yl)carbamate Int-3e

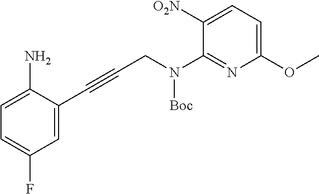 [SMILES: COc1ccc([N+](=O)[O-])c(N(CC#Cc2cc(F)ccc2N)C(=O)OC(C)(C)C)n1]

A mixture of 4-fluoro-2-iodoaniline (5.98 g, 25.2 mmol) and tert-butyl (6-methoxy-3-nitropyridin-2-yl)(prop-2-yn-1-yl)carbamate (7.38 g, 24.0 mmol), in Et₂O (150 mL) was sparged with N₂ (3×), to which was added copper(I) iodide (0.457 g, 2.40 mmol), bis(triphenylphosphine)palladium(II)chloride (0.843 g, 1.20 mmol) and diisopropylamine (16.8 mL, 120 mmol) and the reaction was stirred under N₂ at room temperature for 18 h. The reaction mixture was quenched with sat'd aq. NH₄Cl, diluted with EtOAc, the layers separated, the organic phase washed with sat'd aq. NH₄Cl, H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (40 g) eluting with a 100% heptane to 50% EtOAc-heptanes gradient. Product fractions were combined and evaporated under reduced pressure to afford tert-butyl (3-(2-amino-5-fluorophenyl)prop-2-yn-1-yl)(6-methoxy-3-nitropyridin-2-yl)carbamate Int-3e (8.53 g, 79% yield) as a yellow foam. HPLC/MS 1.27 min (A), [M+H]⁺ 417.1. ¹H NMR (DMSO-d₆, 400 MHz) δ 8.3-8.5 (m, 1H), 6.89 (d, 3H, J=8.8 Hz), 6.5-6.7 (m, 1H), 5.17 (s, 2H), 4.94 (s, 2H), 4.01 (s, 3H), 1.37 (br s, 11H).

### Step A: tert-Butyl (3-(2-amino-5-fluorophenyl)prop-2-yn-1-yl)carbamate Int-3f

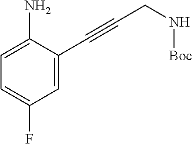 [SMILES: CC(C)(C)OC(=O)NCC#Cc1cc(F)ccc1N]

Following the procedure outlined in Scheme 11, Step A, substituting tert-butyl but-3-yn-1ylcarbamate with tert-butyl prop-2-yn-1-ylcarbamate, tert-butyl (3-(2-amino-5-fluorophenyl)prop-2-yn-1-yl)carbamate Int-3f (0.200 g, 46% yield) was prepared as a viscous oil. HPLC/MS 0.88 min (A), [M+H]⁺ 265.1. ¹H NMR (DMSO-d₆, 400 MHz) δ 7.37 (br s, 1H), 6.85-6.99 (m, 2H), 6.59-6.74 (m, 1H), 5.29 (s, 2H), 4.00 (d, J=5.38 Hz, 2H), 1.38-1.44 (m, 9H).

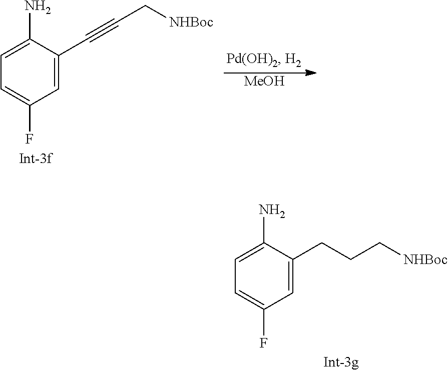 [SMILES: "MeOH" ; CC(C)(C)OC(=O)NCC#Cc1cc(F)ccc1N ; CC(C)(C)OC(=O)NCCCc1cc(F)ccc1N]

### Step A: tert-Butyl (3-(2-amino-5-fluorophenyl)propyl)carbamate Int-32

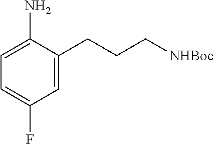 [SMILES: CC(C)(C)OC(=O)NCCCc1cc(F)ccc1N]

To a solution of tert-butyl (3-(2-amino-5-fluorophenyl)prop-2-yn-1-yl)carbamate (3.00 g, 11.4 mmol) in MeOH (28.0 mL) was added Pd(OH)₂—C(2.42 g, 3.45 mmol) and the reaction was stirred under 1 atm H₂ for 18 h. The reaction was filtered through celite, washed with DCM, and the solvent evaporated under reduced pressure. The residue was purified by silica gel flash column chromatography (80 g) eluting with a 100% heptanes to 100% EtOAc gradient. The product fractions were combined and the solvent evaporated under reduced pressure to afford tert-butyl (3-(2-amino-5-fluorophenyl)propyl)carbamate Int-3g (2.10 g, 66% yield) as a white solid. HPLC/MS 0.61 min (A), [M+H]⁺ 269.1. ¹H NMR (CD₃OD, 400 MHz) δ 6.78 (td, 1H, J=1.7, 9.4 Hz), 6.7-6.7 (m, 2H), 3.1-3.2 (m, 2H), 2.5-2.6 (m, 2H), 1.7-1.9 (m, 2H), 1.46 (s, 9H).

### Step A: tert-Butyl (3-(6-amino-2,3-difluorophenyl)prop-2-yn-1-yl)carbamate Int-3h

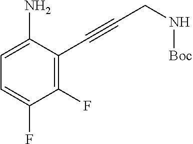 [SMILES: CC(C)(C)OC(=O)NCC#Cc1c(N)ccc(F)c1F]

To a solution of 3,4-difluoro-2-iodoaniline (2.50 g, 9.80 mmol), triphenylphosphine (0.514 g, 1.96 mmol), copper(I) iodide (0.373 g, 1.96 mmol), bis(triphenylphosphine)palladium(II) chloride (0.688 g, 0.980 mmol) and triethylamine (4.10 mL, 29.4 mmol) in DMF (50 mL), purged with N₂, was added tert-butyl prop-2-yn-1-ylcarbamate (2.28 g, 14.7 mmol) and the reaction mixture was heated at 80° C. for 24 h. The reaction mixture was cooled to ambient temperature, diluted with EtOAc, washed with H₂O, the layers separated, the aqueous layer extracted with EtOAc, and the combined extracts washed with brine, dried over MgSO₄, filtered, and the solvent evaporated under reduced pressure. The crude residue was dissolved in DCM, adsorbed onto a silica gel precolumn and purified by silica gel flash column chromatography (220 g), eluting with a 100% heptanes to 100% EtOAc gradient. The product fractions were combined and evaporated under reduced pressure to afford tert-butyl (3-(6-amino-2,3-difluorophenyl)prop-2-yn-1-yl)carbamate Int-3h (2.34 g, 82% yield) as a yellow solid. HPLC/MS 1.03 min (B), [M-^{t}Bu]⁺ 227.1. ¹H NMR (CDCl₃, 400 MHz) δ 6.94 (dt, J=10.27, 8.80 Hz, 1H), 6.37 (ddd, J=9.05, 3.67, 1.96 Hz, 1H), 4.22 (br s, 2H), 1.49 (s, 9H).

### Step B: tert-Butyl (3-(6-amino-2,3-difluorophenyl)propyl)carbamate Int-3i

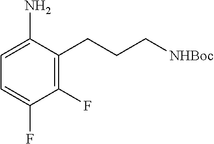 [SMILES: CC(C)(C)OC(=O)NCCCc1c(N)ccc(F)c1F]

Following the procedure outlined in Scheme 14, Step A, substituting tert-butyl (3-(2-amino-5-fluorophenyl)prop-2-yn-1-yl)carbamate with tert-butyl (3-(6-amino-2,3-difluoro-phenyl)prop-2-yn-1-yl)carbamate, tert-butyl (3-(6-amino-2,3-difluorophenyl)propyl)-carbamate Int-3i (491 mg, 50% yield) was prepared as a light pink solid. HPLC/MS 0.79 min (A), [M+H]⁺ 287.1. ¹H NMR (CDCl₃, 400 MHz) δ 6.76-6.84 (m, 1H), 6.35 (ddd, J=8.80, 3.91, 1.96 Hz, 1H), 4.91 (br s, 1H), 3.69 (br s, 2H), 3.14 (br d, J=6.36 Hz, 2H), 2.56-2.61 (m, 2H), 1.71-1.79 (m, 2H), 1.43 (s, 9H).

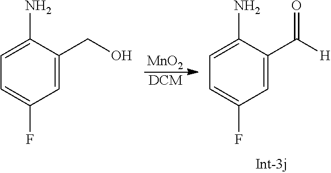

### Step A: 2-Amino-5-fluorobenzaldehyde Int-3i

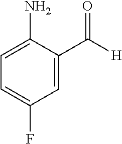

To a solution of (2-amino-5-fluorophenyl)methanol (10.0 g, 70.8 mmol) in DCM (150 ml) was added MnO₂ (12.3 g, 142 mmol) and the reaction mixture stirred at room temperature for 24 h. The reaction mixture was filtered over a pad of celite, the celite washed with DCM and the solvent evaporated under reduced pressure. The crude residue was dissolved in DCM with min. amt MeOH, absorbed onto a silica gel packed precolumn and purified by silica gel flash column chromatography (330 g) eluting with a 100% heptane to 50% EtOAc-hetpanes gradient. Product fractions were combined and evaporated under reduced pressure to afford 2-amino-5-fluorobenzaldehyde Int-3j (3.1 g, 31% yield) as a bright yellow solid. HPLC/MS 0.55 min (A), [M+H]⁺ 139.9. ¹H NMR (DMSO-d₆, 400 MHz) δ 9.79 (s, 1H), 7.39 (dd, 1H, J=3.2, 9.0 Hz), 7.2-7.3 (m, 1H), 7.02 (br s, 2H), 6.79 (dd, 1H, J=4.4, 9.3 Hz).

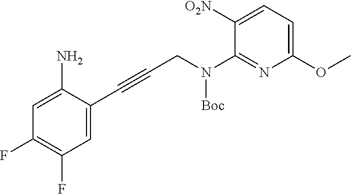 [SMILES: COc1ccc([N+](=O)[O-])c(N(CC#Cc2cc(F)c(F)cc2N)C(=O)OC(C)(C)C)n1]

### Step A: tert-Butyl (3-(2-amino-4,5-difluorophenyl)prop-2-yn-1-yl)(6-methoxy-3-nitropyridin-2-yl) carbamate Int-3k

Following the procedure outlined in Scheme 13, Step C, substituting 4-fluoro-2-iodoaniline with 4,5-difluoro-2-iodoaniline (WO2011006903), tert-butyl (3-(2-amino-4,5-difluorophenyl)prop-2-yn-1-yl)(6-methoxy-3-nitropyridin-2-yl)carbamate Int-3k (3.11 g, 74% yield) was prepared as a light orange foam. HPLC/MS 1.31 min (A), [M-9Bu]⁺ 379.0. ¹H NMR (DMSO-d₆, 400 MHz) δ 8.3-8.5 (m, 1H), 7.0-7.2 (m, 1H), 6.8-7.0 (m, 1H), 6.5-6.7 (m, 1H), 5.44 (s, 2H), 4.93 (br s, 2H), 4.01 (s, 3H), 1.36 (br s, 12H).

### Step A: tert-Butyl (3-(2-bromo-5-fluorophenyl)prop-2-yn-1-yl)carbamate Int-31

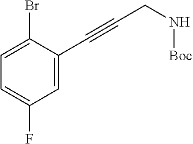 [SMILES: CC(C)(C)OC(=O)NCC#Cc1cc(F)ccc1Br]

Following the procedure outlined in Int-1c-2, substituting tert-butyl but-3-yn-1-ylcarbamate with tert-butyl prop-2-yn-1-ylcarbamate and 2-iodo-6-methoxy-3-nitropyridine with 1-bromo-4-fluoro-2-iodobenzene, and stirring at RT for 23 h, tert-butyl (3-(2-bromo-5-fluorophenyl)prop-2-yn-1-yl)carbamate Int-31 (8.37 g, 77% yield) was prepared as a white solid. HPLC/MS 1.18 min (A), [M-^{t}Bu]+271.9. ¹H NMR (DMSO-d₆, 400 MHz) δ 7.73 (dd, 1H, J=5.4, 9.3 Hz), 7.41 (dd, 2H, J=2.9, 9.3 Hz), 7.22 (dt, 1H, J=2.9, 8.6 Hz), 4.04 (br d, 2H, J=5.4 Hz), 1.41 (s, 9H).

### Step A: tert-Butyl (3-(6-amino-2,3-difluorophenyl)prop-2-yn-1-yl)(6-methoxy-3-nitropyridin-2-yl)carbamate Int-3m

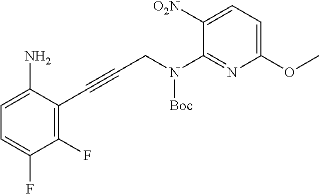 [SMILES: COc1ccc([N+](=O)[O-])c(N(CC#Cc2c(N)ccc(F)c2F)C(=O)OC(C)(C)C)n1]

Following the procedure outlined in Int-3e, substituting 4-fluoro-2-iodoaniline with 3,4-difluoro-2-iodoaniline, and stirring at RT for 65 h, tert-butyl (3-(6-amino-2,3-difluorophenyl)prop-2-yn-1-yl)(6-methoxy-3-nitropyridin-2-yl)carbamate Int-3m (12.6 g, 35% yield) was prepared as a foamy solid. HPLC/MS 1.30 min (A), [M+H]⁺ 435.1. ¹H NMR (DMSO-d₆, 400 MHz): δ 8.40 (d, J=8.8 Hz, 1H), 7.12 (dt, J=10.6, 9.1 Hz, 1H), 6.89 (d, J=9.3 Hz, 1H), 6.44 (ddd, J=9.3, 4.2, 1.7 Hz, 1H), 5.47 (s, 2H), 4.98 (s, 2H), 4.01 (s, 3H), 1.38 (br s, 9H).

### Step A: tert-Butyl (2-((2-bromo-5-fluorobenzyl)oxy)ethyl)carbamate Int-3n

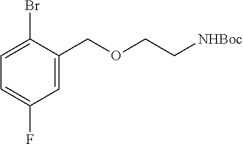 [SMILES: CC(C)(C)OC(=O)NCCOCc1cc(F)ccc1Br]

To a solution of tert-butyl N-(2-hydroxyethyl)carbamate (2.41 g, 14.3 mmol) in DMF (50 mL) was added 60% NaH (0.90 g, 22.4 mmol). After 10 min, 1-bromo-2-(bromomethyl)-4-fluorobenzene (2.00 g, 7.46 mmol) was added, and the reaction mixture slowly warmed to RT. After 1 h, the reaction mixture was quenched with sat'd aq. NH₄Cl solution (250 mL) and extracted with EtOAc (3×100 mL). The combined organic extracts were washed brine, dried over MgSO₄, filtered, and concentrated under reduced pressure. The crude residue was purified by silica gel flash column chromatography (80 g), eluting with a 100% heptanes to 30% EtOAc-heptanes gradient. Product fractions were combined and evaporated under reduced pressure to afford tert-butyl (2-((2-bromo-5-fluorobenzyl)oxy)ethyl)carbamate Int-3n (1.82 g, 66% yield) as a white solid. HPLC/MS 1.15 min (A), [M+H]⁺ 348.1. ¹H NMR (CDCl₃, 400 MHz): δ 7.51 (dd, J=8.6, 5.1 Hz, 1H), 7.24 (dd, J=9.5, 3.2 Hz, 1H), 6.87-6.94 (m, 1H), 4.94 (br s, 1H), 4.56 (s, 2H), 3.66 (t, J=5.1 Hz, 2H), 3.43 (q, J=5.4 Hz, 2H), 1.48 (s, 9H).

### EXAMPLES

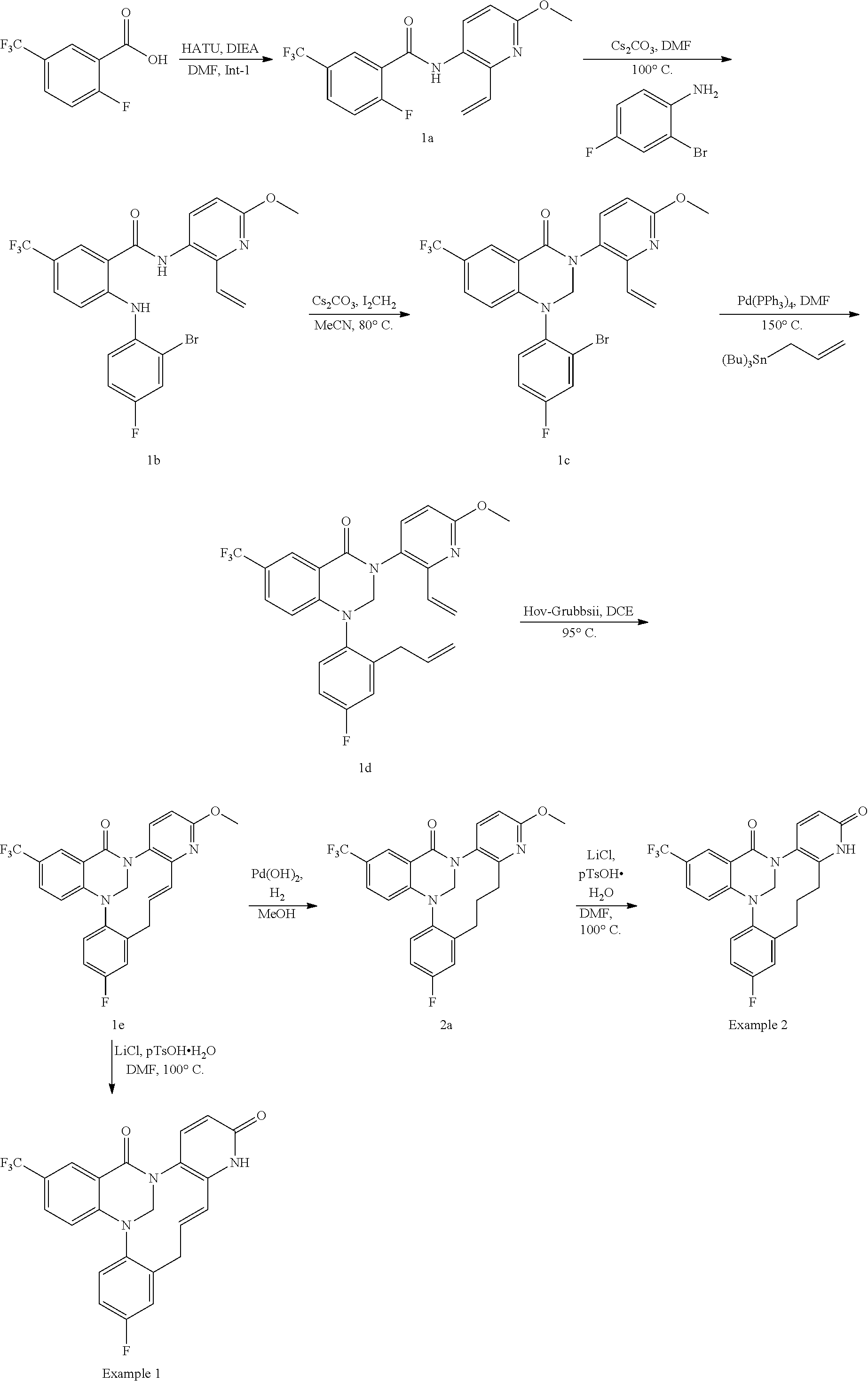

### Examples 1 and 2

### (E)-8-Fluoro-2-(trifluoromethyl)-13-hydro-10H,18H,5,17-methanodibenzo[b,k]pyrido[3,2-f][1,5]diazacyclododecine-14,18-dione Example 1 and 14-Fluoro-8-(trifluoromethyl)-17,18-dihydro-1H,6H-5,11-methanodibenzo[b,k]pyridino[3,2-f][1,5]diazacyclododecine-2,6(16H)-dione

### Example 2

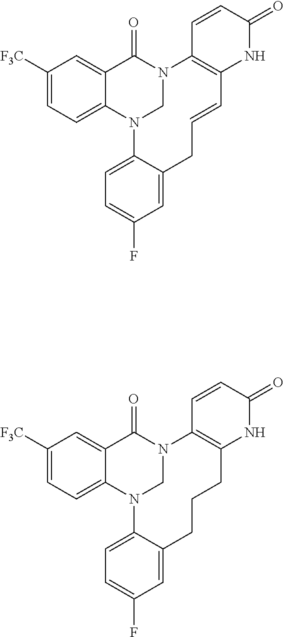 [SMILES: O=C1c2cc(C(F)(F)F)ccc2N2CN1c1ccc(=O)[nH]c1/C=C/Cc1cc(F)ccc12 ; O=C1c2cc(C(F)(F)F)ccc2N2CN1c1ccc(=O)[nH]c1CCCc1cc(F)ccc12]

### Step A: 2-Fluoro-N-(6-methoxy-2-vinylpyridine-3-yl)-5-(trifluormethyl)benzamide 1a

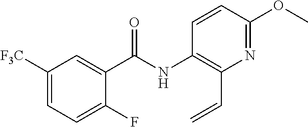 [SMILES: C=Cc1nc(OC)ccc1NC(=O)c1cc(C(F)(F)F)ccc1F]

To a solution of 2-fluoro-5-(trifluoromethyl)benzoic acid (7.00 g, 33.6 mmol) and 6-methoxy-2-vinylpyridin-3-amine Int-1 (5.56 g, 37.0 mmol) in DMF (32 mL) was added DIEA (17.6 mL, 101 mmol) and HATU (19.2 g, 50.5 mmol) at room temperature and the reaction mixture was stirred for 2 h. The reaction mixture was diluted with ice cold water (60 mL), the precipitate filtered and dried under high vacuum to afford 2-fluoro-N-(6-methoxy-2-vinylpyridin-3-yl)-5-(trifluoromethyl)benzamide 1a (8.2 g, 71% yield) of an off-white solid. HPLC/MS 1.20 min (C), [M+H]⁺ 341.2. ¹H NMR (DMSO-d₆, 400 MHz) δ 10.3 (s, 1H), 8.11-8.13 (m, 1H), 7.99-8.03 (m, 1H), 7.74-7.76 (d, 1H), 7.61-7.66 (t, 1H), 6.90-7.01 (m, 1H), 6.81-6.83 (d, 1H), 6.36-6.41 (dd, 1H), 5.49-5.52 (m, 1H), 3.91 (s, 3H).

### Step B: 2-((2-Bromo-4-fluorophenyl)amino)-N-(6-methoxy-2-vinylpyridin-3-yl)-5-(trifluormethyl) benzamide 1b

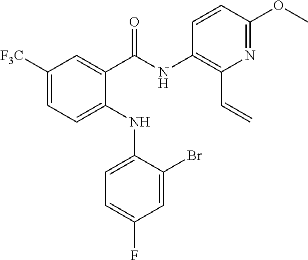 [SMILES: C=Cc1nc(OC)ccc1NC(=O)c1cc(C(F)(F)F)ccc1Nc1ccc(F)cc1Br]

To a solution of 2-fluoro-N-(6-methoxy-2-vinylpyridin-3-yl)-5-(trifluoromethyl)benzamide (8.20 g, 24.1 mmol) and 2-bromo-4-fluoroaniline (3.02 mL, 26.5 mmol) in DMF (50 mL) was added Cs₂CO₃ (15.7 g, 48.2 mmol) and the reaction mixture was stirred at 100° C. for 16 h. The reaction mixture was cooled to ambient temperature and diluted with ice cold water (100 mL), extracted with EtOAc (2×100 mL), the combined organic extracts dried over Na₂SO₄, filtered and the solvent evaporated under reduced pressure. The crude residue was dissolved in DCM, preabsorbed onto a silica gel and purified by silica gel flash column chromatography (120 g) eluting with 5-6% EtOAc-pet. ether. Product fractions were combined and evaporated under reduced pressure to afford 2-((2-bromo-4-fluorophenyl)amino)-N-(6-methoxy-2-vinylpyridin-3-yl)-5-(trifluoromethyl)benzamide 1b (4.5 g, 35% yield) as an off-white solid. HPLC/MS 1.46 min (C), [M+H]⁺ 509.6. ¹H NMR (DMSO-d₆, 400 MHz) δ 10.4 (s, 1H), 10.0 (s, 1H), 8.32 (s, 1H), 7.67-7.73 (m, 3H), 7.55-7.58 (m, 1H), 7.28-7.34 (m, 1H), 7.07-7.09 (d, 1H), 6.77-6.96 (m, 2H), 6.36-6.41 (dd, 1H), 5.47-5.50 (m, 1H), 3.92 (s, 3H).

### Step C: 1-(2-Bromo-4-fluorophenyl)-3-(6-methoxy-2-vinylpyridin-3-yl)-6-(trifluoromethyl)-2,3-dihydroqiuinazolin-4(1H)-one 1c

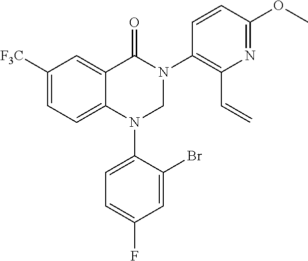 [SMILES: C=Cc1nc(OC)ccc1N1CN(c2ccc(F)cc2Br)c2ccc(C(F)(F)F)cc2C1=O]

To a solution of 2-((2-bromo-4-fluorophenyl)amino)-N-(6-methoxy-2-vinylpyridin-3-yl)-5-(trifluoromethyl)benzamide (4.30 g, 8.43 mmol) in MeCN (50 mL) was added Cs₂CO₃ (10.9 g, 33.7 mmol) and diiodomethane (2.04 mL, 25.3 mmol) and the reaction mixture was stirred at 80° C. for 16 h. The reaction mixture was cooled to ambient temperature and diluted with ice cold water (100 mL), extracted with EtOAc (2×100 mL), the combined organic extracts dried over Na₂SO₄, filtered and the solvent evaporated under reduced pressure. The crude residue was dissolved in DCM, preabsorbed onto a silica gel and purified by silica gel flash column chromatography (120 g) eluting with 15-17% EtOAc-pet. ether. Product fractions were combined and evaporated under reduced pressure to afford 1-(2-bromo-4-fluorophenyl)-3-(6-methoxy-2-vinylpyridin-3-yl)-6-(trifluoromethyl)-2,3-dihydroquinazolin-4(1H)-one 1c (2.42 g, 53% yield) as an off-white solid. HPLC/MS 1.39 min (C), [M+H]⁺ 524.0. ¹H NMR (DMSO-d₆, 400 MHz) S 8.11 (s, 1H), 7.86-7.88 (m, 1H), 7.63-7.75 (m, 3H), 7.45-7.48 (m, 1H), 6.99-7.02 (m, 1H), 6.84-6.86 (m, 1H), 6.36-6.44 (m, 2H), 5.65-5.68 (m, 1H), 5.47-5.50 (m, 1H), 5.22-5.27 (m, 1H), 4.79-4.81 (d, 1H), 3.92 (s, 3H).

### Step D: 1-(2-Allyl-4-fluorophenyl)-3-(6-methoxy-2-vinylpyridin-3-yl)-6-(trifluoromethyl)-2,3-dihydroqiuinazolin-4(1H)-one 1d

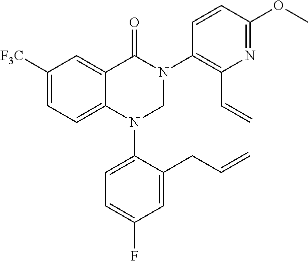 [SMILES: C=CCc1cc(F)ccc1N1CN(c2ccc(OC)nc2C=C)C(=O)c2cc(C(F)(F)F)ccc21]

To a solution of 1-(2-bromo-4-fluorophenyl)-3-(6-methoxy-2-vinylpyridin-3-yl)-6-(trifluoromethyl)-2,3-dihydroquinazolin-4(1H)-one (2.32 g, 4.44 mmol) in DMF (20 mL) was added allyltributylstannane (2.75 mL, 8.88 mmol) and Pd(PPh₃)₄ (0.257 g, 0.222 mmol) at room temperature and the reaction mixture was heated at 150° C. under microwave irradiation for 1 h. The reaction mixture was cooled to ambient temperature, diluted with ice cold water (10 mL), extracted with EtOAc (2×50 mL), and the combined organic extracts dried over Na₂SO₄, filtered and the solvent evaporated under reduced pressure. The crude residue was dissolved in DCM, preabsorbed onto a silica gel and purified by silica gel flash column chromatography (120 g) eluting with 18-20% EtOAc-pet. ether. Product fractions were combined and evaporated under reduced pressure to afford 1-(2-allyl-4-fluorophenyl)-3-(6-methoxy-2-vinylpyridin-3-yl)-6-(trifluoromethyl)-2,3-dihydroquinazolin-4(1H)-one 1d (1.80 g, 73% yield) as colorless liquid. HPLC/MS 1.46 min (C), [M+H]⁺ 483.6. ¹H NMR (DMSO-d₆, 400 MHz) δ 8.09 (s, 1H), 7.68-7.73 (m, 2H), 7.46-7.65 (m, 1H), 7.27-7.29 (m, 1H), 6.84-6.87 (m, 2H), 6.34-6.42 (m, 2H), 5.82-5.98 (m, 1H), 5.66-5.76 (m, 1H), 5.50-5.51 (m, 1H), 4.97-5.07 (m, 3H), 4.68-4.70 (m, 1H), 3.91 (s, 3H), 3.38-3.40 (m, 2H).

### Step E: (E)-8-Fluoro-14-methoxy-2-(trifluoromethyl)-10H,18H, 5,17-methanodibenzo[b,k]pyrido[3,2-f][1,5]diazacyclododecin-18-one 1e

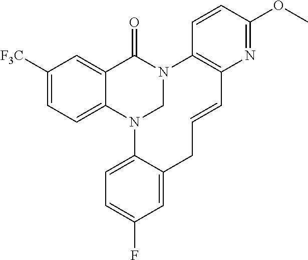 [SMILES: COc1ccc2c(n1)/C=C/Cc1cc(F)ccc1N1CN2C(=O)c2cc(C(F)(F)F)ccc21]

To a solution of 1-(2-allyl-4-fluorophenyl)-3-(6-methoxy-2-vinylpyridin-3-yl)-6-(trifluoromethyl)-2,3-dihydroquinazolin-4(1H)-one (200 mg, 0.414 mmol) in DCE (300 mL) was added Hoveyda-Grubbsii (51.8 mg, 0.083 mmol) under N₂ atmosphere, and the reaction mixture was heated at 80° C. for 4 h. The reaction mixture was cooled to ambient temperature, filtered through celite, washed with EtOAc (2×10 mL), and the organic layer dried over Na₂SO₄, filtered and the solvent evaporated under reduced pressure. The crude product was dissolved in DCM, adsorbed onto silica gel and purified by silica gel flash column chromatography (10 g) eluting with 19-23% EtOAc-pet. ether. Product fractions were combined and evaporated under reduced pressure to afford (E)-8-fluoro-14-methoxy-2-(trifluoromethyl)-10H,18H, 5,17-methanodibenzo[b,k]pyrido[3,2-f][1,5]diazacyclododecin-18-one 1e (140 mg, 72% yield) of a green oil. HPLC/MS 1.29 min (C), [M+H]⁺ 456.0.

### Step F: (E)-8-Fluoro-2-(trifluoromethyl)-13-hydro-10H, 18H,-5,17-methanodibenzo[b,k]pyrido[3,2-f][1,5]diazacyclododecine-14,18-dione Example 1

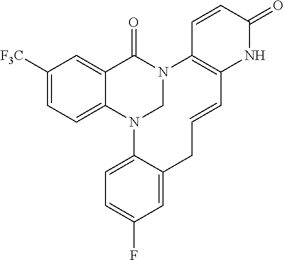 [SMILES: O=C1c2cc(C(F)(F)F)ccc2N2CN1c1ccc(=O)[nH]c1/C=C/Cc1cc(F)ccc12]

To a solution of (E)-8-fluoro-14-methoxy-2-(trifluoromethyl)-10H,18H-5,17-methanodibenzo[b,k]pyrido [3,2-][1,5]diazacyclododecin-18-one (100 mg, 0.220 mmol) in DMF (3 mL) was added LiCl (55.8 mg, 1.32 mmol) and p-TsOH (251 mg, 1.32 mmol) and the reaction mixture was stirred at 100° C. for 12 h. The reaction mixture was diluted with ice cold water (50 mL), extracted with EtOAc (2×30 mL), the combined organic extracts washed with brine, dried over Na₂SO₄, filtered and the solvent evaporated under reduced pressure. The crude residue was purified by semi-prep HPLC (Sunfire C_{18, 19×150} mm, 5 μM) eluting with a 40% MeCN—H₂O (0.1% HCO₂H) to 100% MeCN (0.1% HCO₂H) over 13 min. Product fractions were combined and evaporated under reduced pressure to afford (E)-8-fluoro-2-(trifluoromethyl)-13-hydro-10H,18H-5,17-methanodibenzo[b,k]pyrido[3,2-f][1,5]diazacyclododecine-14,18-dione (16.3 mg, 17% yield) as an off-white solid. HPLC/MS 1.02 min (C), [M+H]⁺ 442.2. ¹H NMR (DMSO-d₆, 400 MHz) δ 11.74 (bs, 1H), 8.08 (s, 1H), 7.62 (dd, J=1.60, 8.60 Hz, 1H), 7.55-7.43 (m, 3H), 7.36-7.27 (m, 1H), 6.67 (bs, 1H), 6.36-6.23 (m, 2H), 6.01 (bs, 1H), 5.40 (d, J=12.00, 1H), 4.85 (bs, 1H), 3.62-3.36 (m, 2H).

### Step G: 14-fluoro-2-methoxy-8-(trifluoromethyl)-17,18-dihydro-6H,16H-5,11-methanodibenzo[b,k]pyridino[3,2-f][1,5]diazacyclododecine-6-dione 2a

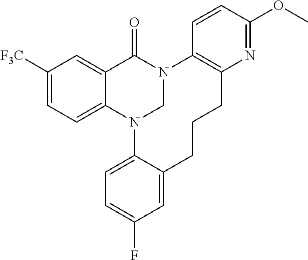 [SMILES: COc1ccc2c(n1)CCCc1cc(F)ccc1N1CN2C(=O)c2cc(C(F)(F)F)ccc21]

To a solution of (E)-8-fluoro-14-methoxy-2-(trifluoromethyl)-10H,18H-5,17-methanodibenzo [b,k]pyrido[3,2-f][1,5]diazacyclododecin-18-one (140 mg, 0.307 mmol) in MeOH (5 mL) was added PdOH₂ (21.6 mg, 0.031 mmol) and the reaction mixture was stirred under an atmosphere of H₂ (5 kg pressure) for 16 h. The reaction mixture was filtered through celite, washed with MeOH (2×30 mL), and the solvent evaporated under reduced pressure to afford 14-fluoro-2-methoxy-8-(trifluoromethyl)-17,18-dihydro-6H,16H-5,11-methanodibenzo[b,k]pyridino[3,2-f][1,5]diazacyclododecine-6-dione 2a (130 mg, 84% yield) as a brown solid. HPLC/MS 1.31 min (C), [M+H]⁺ 457.8.

### Step H: 14-Fluoro-8-(trifluoromethyl)-17,18-dihydro-1H,6H-5,11-methanodibenzo[b,k]pyridino[3,2-f][1,5]diazacyclododecine-2,6(16H)-dione Example 2

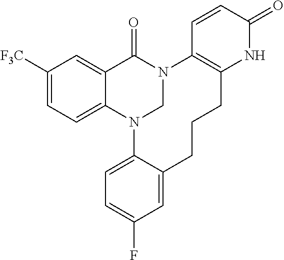 [SMILES: O=C1c2cc(C(F)(F)F)ccc2N2CN1c1ccc(=O)[nH]c1CCCc1cc(F)ccc12]

To a solution of 14-fluoro-2-methoxy-8-(trifluoromethyl)-17,18-dihydro-6H,16H-5,11-methano dibenzo[b,k]pyrido[3,2-f][1,5]diazacyclododecin-6-one (130 mg, 0.284 mmol) in DMF (5 mL) was added LiCl (72.3 mg, 1.71 mmol) and p-TsOH (324 mg, 1.71 mmol) and the reaction mixture was stirred at 100° C. for 5 h. The reaction mixture was diluted with ice cold water (50 mL), extracted with EtOAc (2×30 mL), the combined extracts washed with brine, dried over Na₂SO₄, filtered and the solvent evaporated under reduced pressure. The crude residue was purified by semi-prep HPLC (Sunfire C_{18, 19×150} mm, 5 μM) eluting with a 25% MeCN—H₂O (0.10% HCO₂H) to 70% MeCN—H₂O (0.10% HCO₂H) gradient over 10 min, then to 97% MeCN—H₂O (0.10% HCO₂H) over 9 min. Product fractions were combined and evaporated under reduced pressure to afford 14-fluoro-8-(trifluoromethyl)-17,18-dihydro-1H,6H-5,11-methanodibenzo [b,k]pyridino[3,2-f][1,5]diazacyclododecine-2,6(16H)-dione (27.8 mg, 22% yield) as white solid. HPLC/MS 1.03 min (C), [M+H]⁺ 444.2. ¹H NMR (DMSO-d₆, 400 MHz) δ 11.72 (bs, 1H), 8.13 (s, 1H), 7.68-7.23 (m, 5H), 6.75-6.48 (m, 1H), 6.28-6.13 (m, 1H), 5.49-5.20 (m, 1H), 4.82 (d, J=12.00 Hz, 1H), 2.90-2.62 (m, 2H), 2.40-2.34 (m, 2H), 1.84-1.78 (m, 1H), 1.39-1.24 (m, 1H).

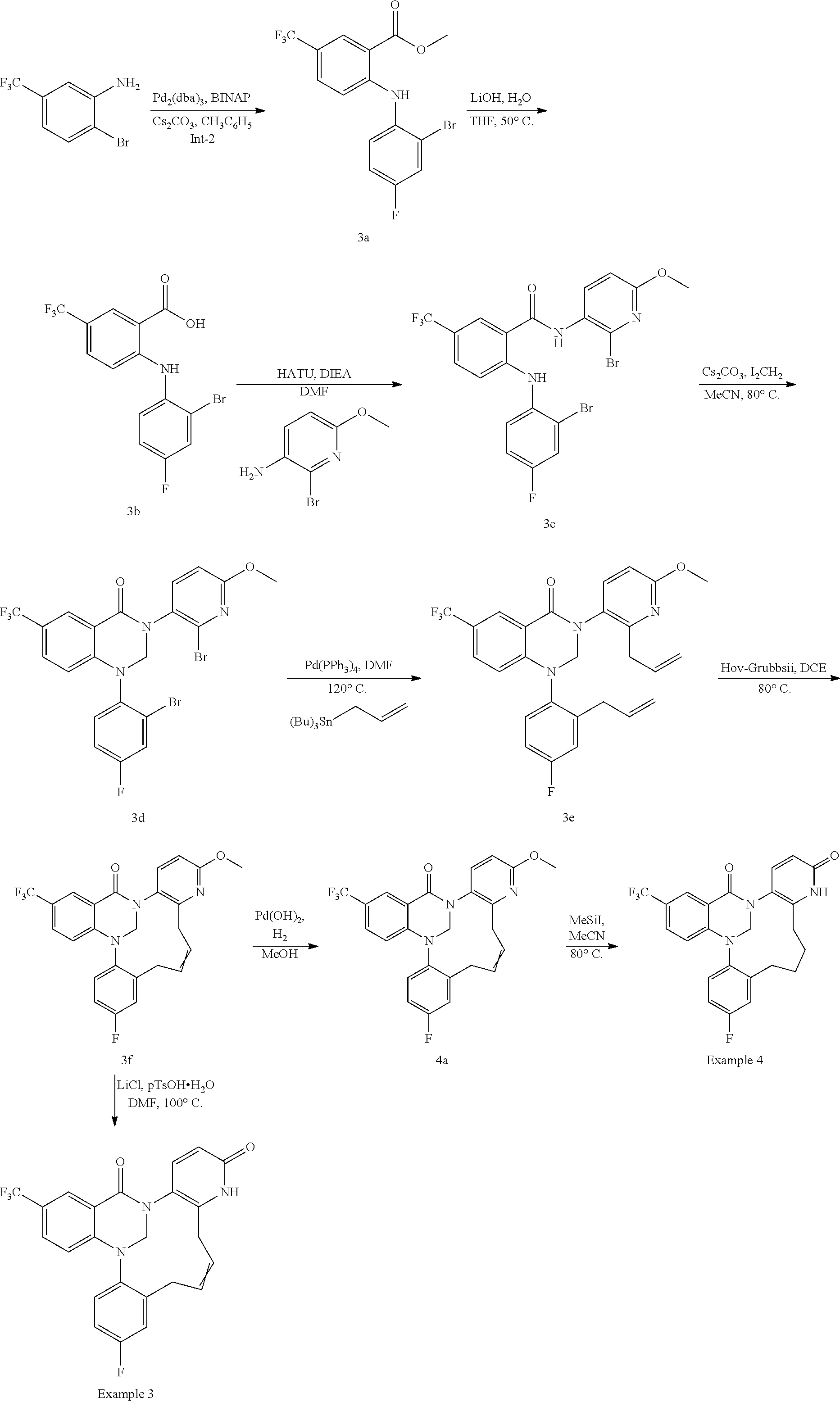

### Examples 3 and 4

### 8-Fluoro-2-(trifluoromethyl)-10,13-dihydro-19H-5,18-methanodibenzo[b,l]pyrido[3,2-f][1.5]diaza cyclotridecine-15,19(14HM-dione Example 3 and 8-fluoro-2-(trifluoromethyl)-10,11,12,13-tetrahydro-19H-5,18-methanodibenzor[b,l]pyridino[3,2-f][1,5]diazacyclotridecine-15,19(14H₁)-dione

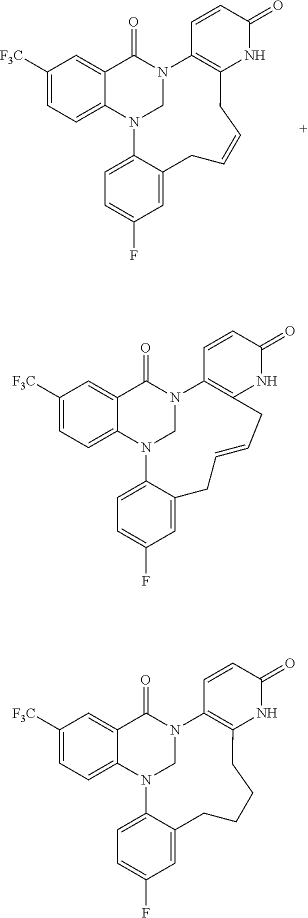 [SMILES: O=C1c2cc(C(F)(F)F)ccc2N2CN1c1ccc(=O)[nH]c1C/C=C\Cc1cc(F)ccc12 ; O=C1c2cc(C(F)(F)F)ccc2N2CN1c1ccc(=O)[nH]c1C/C=C/Cc1cc(F)ccc12 ; O=C1c2cc(C(F)(F)F)ccc2N2CN1c1ccc(=O)[nH]c1CCCCc1cc(F)ccc12]

### Step A: Methyl 2-((2-bromo-4-fluorophenyl amino)-5-(trifluoromethyl)benzoate 3a

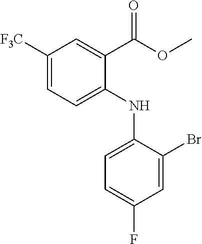 [SMILES: COC(=O)c1cc(C(F)(F)F)ccc1Nc1ccc(F)cc1Br]

To a mixture of methyl 2-bromo-5-(trifluoromethyl)benzoate Int-2 (2.50 g, 8.83 mmol), 2-bromo-4-fluoroaniline (1.68 g, 8.83 mmol), and Cs₂CO₃ (4.32 g, 13.3 mmol) was added toluene (60 mL), the reaction mixture purged with N₂, to which was added BINAP (0.550 g, 0.883 mmol) and Pd₂(dba)₃ (0.404 g, 0.442 mmol), and the reaction mixture purged further with N₂, then heated at 100° C. for 16 h. The reaction mixture was cooled to ambient temperature, filtered over celite, the celite washed with EtOAc and the organics evaporated under reduced pressure. The crude residue was dissolved in DCM, preabsorbed onto a silica gel prepacked column and purified by silica gel flash column chromatography (100 g) eluting with 5% EtOAc-pet ether. Product fractions were combined and evaporated under reduced pressure to afford methyl 2-((2-bromo-4-fluorophenyl)amino)-5-(trifluoromethyl)benzoate 3a (2.0 g, 33% yield) as an off-white solid, in 57% purity. HPLC/MS 1.44 min (C), [M+H]⁺ 392.0.

### Step B: 2-((2-Bromo-4-fluorophenyl)amino)-5-(trifluoromethyl)benzoic acid 3b

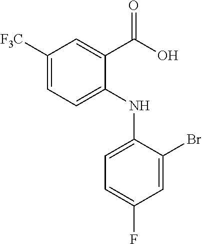 [SMILES: O=C(O)c1cc(C(F)(F)F)ccc1Nc1ccc(F)cc1Br]

To methyl 2-((2-bromo-4-fluorophenyl)amino)-5-(trifluoromethyl)benzoate (4.10 g, 10.5 mmol), was added THF (30 mL) followed by a solution of LiOH (2.63 g, 62.7 mmol) in H₂O (10 mL), dropwise, and the reaction mixture was stirred at 60° C. for 3 h. The reaction mixture was allowed to cool to ambient temperature, concentrated under reduced pressure, and the residue dissolved in H₂O (100 mL), acidified with 1.5N HCl to pH2, and extracted with EtOAc (2×100 mL). The combined organic extracts were dried over Na₂SO₄, filtered and the solvent evaporated under reduced pressure to afford 2-((2-bromo-4-fluorophenyl)amino)-5-(trifluoromethyl)benzoic acid 3b (4.0 g, 78% yield) as a yellow solid, in 77% purity. HPLC/MS 1.37 min (C), [M+H]⁺ 378.0. ¹H NMR (DMSO-d₆, 400 MHz) δ 13.8 (br s, 1H), 9.92 (s, 1H), 8.05-8.15 (m, 1H), 7.75-7.99 (m, 1H), 7.57-7.67 (m, 2H), 7.32-7.37 (m, 1H), 6.89-7.09 (m, 1H).

### Step C: 2-((2-Bromo-4-fluorophenyl)amino)-N-(2-bromo-6-methoxypyridin-3-yl)-5-(trifluoromethyl) benzamide 3c

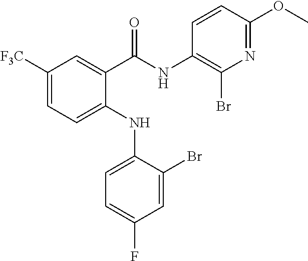 [SMILES: COc1ccc(NC(=O)c2cc(C(F)(F)F)ccc2Nc2ccc(F)cc2Br)c(Br)n1]

To 2-((2-bromo-4-fluorophenyl)amino)-5-(trifluoromethyl)benzoic acid (4.00 g, 10.6 mmol) in DMF (50 mL) was added HATU (6.03 g, 15.9 mmol), followed by DIEA (5.54 mL, 31.7 mmol) and the reaction mixture was stirred at room temperature for 20 min, to which was added 2-bromo-6-methoxypyridin-3-amine (2.58 g, 12.7 mmol) in DMF (10 mL), dropwise, and the reaction mixture was stirred for at room temperature for 16 h. The reaction mixture was quenched with ice water (500 mL), extracted with EtOAc (2×250 mL), and the combined extracts washed with H₂O (250 mL), brine, dried over Na₂SO₄, filtered and the solvent evaporated under reduced pressure. The crude residue was dissolved in DCM, absorbed onto silica gel and purified by silica gel flash column chromatography (100 g) eluting with 10% EtOAc-pet ether. Pure product fractions were combined and evaporated under reduced pressure to afford 2-((2-bromo-4-fluorophenyl)amino)-N-(2-bromo-6-methoxypyridin-3-yl)-5-(trifluoromethyl)benzamide 3c (1.75 g, 27% yield) as a yellow solid. HPLC/MS 1.45 min (C), [M+H]⁺ 562.0. ¹H NMR (DMSO-d₆, 400 MHz) δ 10.51 (s, 1H), 9.97 (s, 1H), 8.30 (s, 1H), 7.85-7.87 (d, 1H), 7.67-7.75 (m, 2H), 7.56-7.59 (m, 1H), 7.29-7.34 (m, 1H), 7.07-7.09 (d, 1H), 6.95-6.97 (d, 1H), 3.89 (s, 3H).

### Step D: 1-(2-Bromo-4-fluorophenyl)-3-(2-bromo-6-methoxypyridin-3-yl)-6-(trifluoromethyl)-2,3-dihydroquinazolin-4(11H)-one 3d

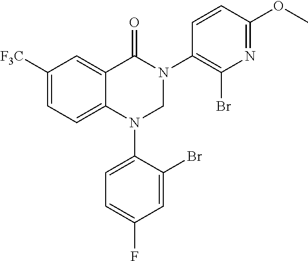 [SMILES: COc1ccc(N2CN(c3ccc(F)cc3Br)c3ccc(C(F)(F)F)cc3C2=O)c(Br)n1]

To a solution of 2-((2-bromo-4-fluorophenyl)amino)-N-(2-bromo-6-methoxypyridin-3-yl)-5-(trifluoromethyl)benzamide (1.75 g, 3.11 mmol) in MeCN (30 mL), under N₂, was added Cs₂CO₃ (4.05 g, 12.4 mmol) and diiodomethane (0.752 mL, 9.32 mmol), dropwise, and the reaction mixture was heated to 80° C. for 20 h. The reaction mixture was quenched with ice-water (100 mL), extracted with EtOAc (2×100 mL), and the combined organic extracts washed with H₂O (100 mL), brine, Na₂SO₄, filtered and the solvent evaporated under reduced pressure. The crude residue was dissolved in DCM, absorbed onto silica gel and purified by silica gel flash column chromatography (100 g) eluting with 10% EtOAc-pet ether. Pure product fractions were combined and evaporated under reduced pressure to afford 1-(2-bromo-4-fluorophenyl)-3-(2-bromo-6-methoxypyridin-3-yl)-6-(trifluoromethyl)-2,3-dihydroquinazolin-4(1H)-one 3d (1.4 g, 75% yield) as an off-white solid. HPLC/MS 2.55 min (E), [M+H]⁺ 573.8. ¹H NMR (DMSO-d₆, 400 MHz) δ 8.12 (s, 1H), 7.85-7.91 (m, 2H), 7.63-7.72 (m, 2H), 7.42-7.48 (m, 1H), 6.97-7.00 (d, 1H), 6.41-6.53 (dd, 1H), 4.45-5.62 (ddd, 2H), 3.87 (s, 3H).

### Step E: 1-(2-Allyl-4-fluorophenyl)-3-(2-allyl-6-methoxypyridin-3-yl)-6-(trifluoromethyl)-2,3-dihydro quinazolin-4(11H)-one 3e

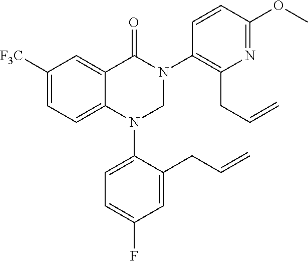 [SMILES: C=CCc1cc(F)ccc1N1CN(c2ccc(OC)nc2CC=C)C(=O)c2cc(C(F)(F)F)ccc21]

To a solution of 1-(2-bromo-4-fluorophenyl)-3-(2-bromo-6-methoxypyridin-3-yl)-6-(trifluoromethyl)-2,3-dihydroquinazolin-4(1H)-one (0.8 g, 1.39 mmol) in DMF (10 mL), under N₂, was added allyltributylstannane (1.73 mL, 5.56 mmol) and Pd(Ph₃P)₄ (0.241 g, 0.209 mmol) and the reaction mixture was heated at 120° C. for 2 h. The reaction mixture was allowed to cool to ambient temperature, quenched with ice-water (200 mL), extracted with EtOAc (2×100 mL), and the combined organic extract was washed with H₂O (100 mL), brine, dried over Na₂SO₄, filtered and the solvent evaporated under reduced pressure. The crude residue was dissolved in DCM, absorbed onto silica gel and purified by silica gel flash column chromatography (100 g) eluting with 20% EtOAc-pet ether. Pure product fractions were combined and evaporated under reduced pressure to afford 1-(2-allyl-4-fluorophenyl)-3-(2-allyl-6-methoxypyridin-3-yl)-6-(trifluoromethyl)-2,3-dihydroquinazolin-4(1H)-one 3e (0.6 g, 84% yield) as a colorless gum. HPLC/MS 1.38 min (C), [M+H]⁺ 498.3. ¹H NMR (DMSO-d₆, 400 MHz) δ 8.09 (s, 1H), 7.65-7.68 (m, 2H), 7.40-7.48 (m, 1H), 7.25-7.27 (m, 2H), 6.76-6.79 (d, 1H), 6.28-6.41 (dd, 1H), 5.81-6.12 (m, 2H), 4.72-5.68 (m, 6H), 3.86 (s, 3H), 3.36-3.46 (m, 4H).

### Step F: 8-Fluoro-15-methoxy-2-(trifluoromethyl)-10,13-dihydro-19H-5,18-methanodibenzo[b,l]pyrido[3,2-f][1,5]diazacyclotridecin-19-one 3f

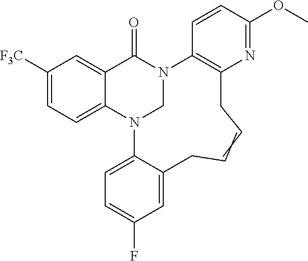 [SMILES: COc1ccc2c(n1)CC=CCc1cc(F)ccc1N1CN2C(=O)c2cc(C(F)(F)F)ccc21]

A 250-mL, sealed tube fitted with a magnetic stir-bar was charged with 1-(2-allyl-4-fluorophenyl)-3-(2-allyl-6-methoxypyridin-3-yl)-6-(trifluoromethyl)-2,3-dihydroquinazolin-4(1H)-one (0.3 g, 0.603 mmol). DCE (70 mL) and Hoveyda-Grubbsii (0.076 g, 0.121 mmol), while under N₂ atmosphere, were added and the light green reaction mixture was heated at 80° C. for 16 h. The reaction mixture was allowed to cool to ambient temperature and filtered over celite, washed with DCM (50 mL) and the solvent evaporated under reduced pressure. The crude residue was dissolved in DCM, absorbed onto silica gel and purified by silica gel flash column chromatography (25 g) eluting with 15% EtOAc-pet ether. Pure product fractions were combined and evaporated under reduced pressure to afford 8-fluoro-15-methoxy-2-(trifluoromethyl)-10,11,12,13-tetrahydro-19H-5,18-methanodibenzo[b,l]pyrido[3,2-f][1,5]diazacyclotridecin-19-one 3f (200 mg, 68% yield) as a light green solid. HPLC/MS 1.36 min (C), [M+H]⁺ 470.0. ¹H NMR (DMSO-d₆, 400 MHz) δ 8.12 (s, 1H), 7.66-7.72 (m, 2H), 7.48-7.57 (m, 2H), 7.23-7.28 (m, 1H), 6.77-6.79 (d, 1H), 6.50-6.52 (d, 1H), 5.48-5.76 (m, 2H), 5.37-5.39 (d, 1H), 4.28-4.32 (m, 2H), 4.17-4.26 (m, 1H), 3.89 (s, 3H), 3.09-3.12 (d, 1H), 3.01-3.04 (d, 1H).

### Step G: 8-Fluoro-2-(trifluoromethyl)-10,13-dihydro-19H-5,18-methanodibenzo[b,l]pyrido[3,2-f][1,5]diazacyclotridecine-15,19(14H)-dione Example 3

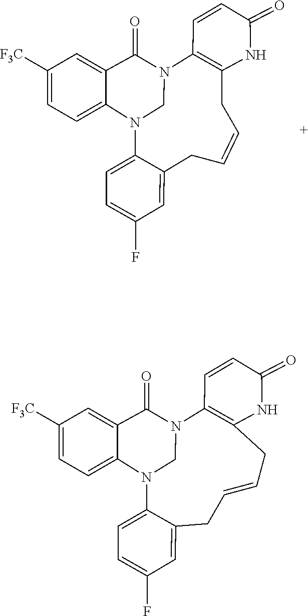 [SMILES: O=C1c2cc(C(F)(F)F)ccc2N2CN1c1ccc(=O)[nH]c1C/C=C\Cc1cc(F)ccc12 ; O=C1c2cc(C(F)(F)F)ccc2N2CN1c1ccc(=O)[nH]c1C/C=C/Cc1cc(F)ccc12]

Following the steps in Example 1, Step F, stirring the reaction mixture at 100° C. for 3 h, 8-fluoro-2-(trifluoromethyl)-10,13-dihydro-19H-5,18-methanodibenzo[b,l]pyrido[3,2-f][1,5]diazacyclotridecine-15,19(14H)-dione (40 mg, 31% yield) was isolated as an off-white solid. HPLC/MS 1.08 min (C), [M+H]⁺ 456.0. ¹H NMR (DMSO-d₆, 400 MHz) δ 12.07 (s, 1H), 8.10 (s, 1H), 7.67 (d, J=2.00 Hz, 8.80 Hz, 1H), 7.56 (d, J=2.80, 9.40 Hz, 1H), 7.50-7.43 (m, 2H), 7.25 (dd, J=2.80, 8.40 Hz, 1H), 6.51 (d, J=8.80 Hz, 1H), 6.38 (d, J=9.20 Hz, 1H), 5.52-5.44 (m, 2H), 5.34 (d, J=10.40 Hz, 1H), 4.25 (d, J=10.00 Hz, 1H), 4.17-4.14 (t, J=12.40 Hz, 1H), 4.11-4.02 (t, J=11.20 Hz, 1H), 3.07 (d, J=14.40 Hz, 1H), 2.93 (d, J=14.00 Hz, 1H).

### Step H: 8-Fluoro-15-methoxy-2-(trifluoromethyl)-10,11,12,13-tetrahydro-19H-5,18-methanodibenzo [b,l]pyrido[3,2-f][1,5]diazacyclotridecin-19-one 4a

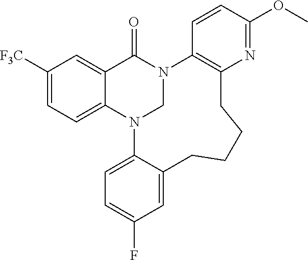 [SMILES: COc1ccc2c(n1)CCCCc1cc(F)ccc1N1CN2C(=O)c2cc(C(F)(F)F)ccc21]

To a solution of 8-fluoro-15-methoxy-2-(trifluoromethyl)-10,11,12,13-tetrahydro-19H-5,18-methanodibenzo[b,l]pyrido[3,2-f][1,5]diaza cyclotridecin-19-one (200 mg, 0.426 mmol) in MeOH (5 mL) purged with N₂, was added 20% Pd(OH)₂ (90 mg, 0.128 mmol), and the reaction mixture was stirred under H₂ atmosphere (1 atm) for 3 h. The reaction mixture was filtered over celite, washed with MeOH (150 mL), and the solvent evaporated under reduced pressure. The crude residue was dissolved in DCM, absorbed onto silica gel and purified by silica gel flash column chromatography (25 g) eluting with 20% EtOAc-pet ether. Pure product fractions were combined and evaporated under reduced pressure to afford 8-fluoro-15-methoxy-2-(trifluoromethyl)-10,11,12,13-tetrahydro-19H-5,18-methanodibenzo[b,l]pyrido[3,2-f][1,5]diazacyclotridecin-19-one 4a (140 mg, 69% yield) as an off-white solid. HPLC/MS 1.37 min (C), [M+H]⁺ 472.2. ¹H NMR (DMSO-d₆, 400 MHz) δ 8.11 (s, 1H), 7.66-7.69 (d, 2H), 7.44-7.48 (m, 1H), 7.35-7.38 (m, 1H), 7.19-7.24 (m, 1H), 6.73-6.75 (d, 1H), 6.40-6.42 (d, 1H), 5.67-5.69 (d, 1H), 4.71-4.74 (d, 1H), 3.84 (s, 3H), 2.72-2.85 (m, 2H), 2.29-2.38 (m, 2H), 1.64-1.67 (m, 2H), 1.40-1.56 (m, 1H), 1.32 (m, 1H).

### Step I: 8-Fluoro-2-(trifluoromethyl)-10,11,12,13-tetrahydro-19H-5,18-methanodibenzo[b,l]pyridino[3,2-f][1,5]diazacyclotridecine-15,19(14H)-dione Example 4

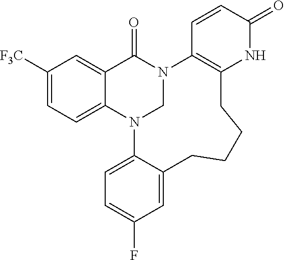 [SMILES: O=C1c2cc(C(F)(F)F)ccc2N2CN1c1ccc(=O)[nH]c1CCCCc1cc(F)ccc12]

To a solution of 8-fluoro-15-methoxy-2-(trifluoromethyl)-10,11,12,13-tetrahydro-19H-5,18-methanodibenzo[b,l]pyrido[3,2-f][1,5]diazacyclotridecin-19-one (140 mg, 0.297 mmol) in MeCN (5 mL), cooled to 0° C. under N₂, was added iodotrimethylsilane (0.121 mL, 0.891 mmol), dropwise, and the reaction mixture was stirred at 80° C. for 3 h. The reaction mixture was allowed to cool to ambient temperature and quenched with sat'd aq. Na₂S₂O₃ solution (20 mL). The aqueous layer was extracted with EtOAc (3×25 mL), the combined organic extracts dried over Na₂SO₄, filtered and the solvent evaporated under reduced pressure. The crude residue was dissolved in DCM, absorbed onto silica gel and purified by silica gel flash column chromatography (25 g) eluting with 10% EtOAc-pet ether. Pure product fractions were combined and evaporated under reduced pressure. The solid was freeze dried to afford 8-fluoro-2-(trifluoromethyl)-10,11,12,13-tetrahydro-19H-5,18-methanodibenzo[b,l]pyridino[3,2-f][1,5]diazacyclotridecine-15,19(14H)-dione (80 mg, 59% yield) as a white solid. HPLC/MS 1.04 min (C), [M+H]⁺ 458.0. ¹H NMR (400 MHz, DMSO-d₆) δ 11.85 (s, 1H), 8.10 (s, 1H), 7.66 (dd, J=2.00, 8.80 Hz, 1H), 7.47-7.41 (m, 3H), 7.26-7.21 (m, 1H), 6.40 (d, J=8.40 Hz, 1H), 6.21 (d, J=9.20 Hz, 1H), 5.58 (d, J=11.60 Hz, 1H), 4.71 (d, J=11.60 Hz, 1H), 2.71-2.64 (m, 1H), 2.58-2.51 (m, 1H), 2.38-2.34 (m, 1H), 2.26-2.20 (m, 1H), 1.64-1.63 (m, 2H), 1.42 (in, 2H).

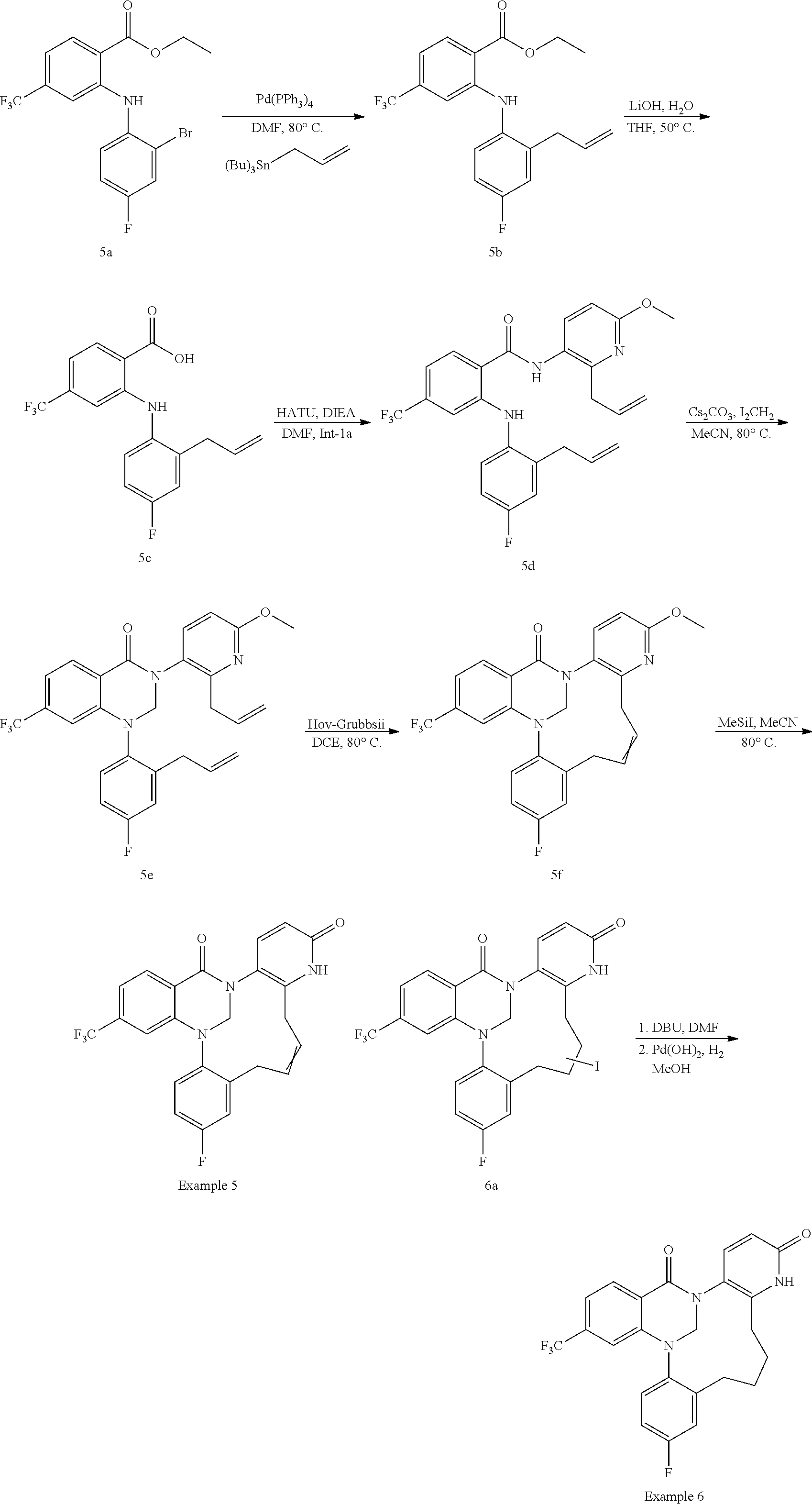

### Examples 5 and 6

### 8-Fluoro-3-(trifluoromethyl)-10,13-dihydro-19H-5,18-methanodibenzo[b,l]pyrido[3,2f][1,5]diazacyclo tridecin-15,19(14H)-dione Example 5 and 8-fluoro-3-(trifluoromethyl)-10,11,12,13-tetrahydro-19H-5,18-methanodibenzo[b,l]pyridino[3,2-f][1,5]diazacyclotridecine-15,19(14H)-dione Example 6

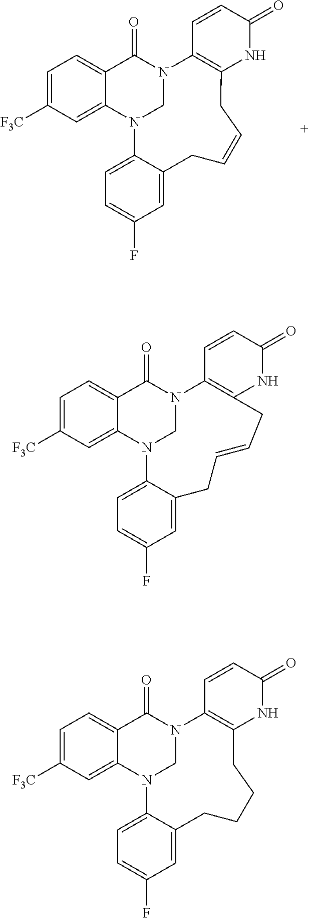 [SMILES: O=C1c2ccc(C(F)(F)F)cc2N2CN1c1ccc(=O)[nH]c1C/C=C\Cc1cc(F)ccc12 ; O=C1c2ccc(C(F)(F)F)cc2N2CN1c1ccc(=O)[nH]c1C/C=C/Cc1cc(F)ccc12 ; O=C1c2ccc(C(F)(F)F)cc2N2CN1c1ccc(=O)[nH]c1CCCCc1cc(F)ccc12]

### Step A: Ethyl 2-((2-bromo-4-fluorophenyl)amino)-4-(trifluoromethyl)benzoate 5a

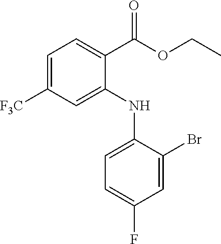 [SMILES: CCOC(=O)c1ccc(C(F)(F)F)cc1Nc1ccc(F)cc1Br]

Following the steps in Example 3, Step A, substituting Int-2 with Int-2a, ethyl 2-((2-bromo-4-fluorophenyl)amino)-4-(trifluoromethyl)benzoate 5a (2.5 g, 60% yield) was prepared as a yellow oil. HPLC/MS 3.15 min (F), [M+H]⁺ 406.0. ¹H NMR (400 MHz, DMSO-d₆) δ 9.45 (s, 1H), 8.11-8.16 (m, 2H), 7.76-7.79 (d, 1H), 7.56-7.59 (m, 1H), 7.33-7.39 (m, 1H), 6.98 (s, 1H), 4.35-4.41 (q, 2H), 1.39-1.38 (t, 3H).

### Step B: Ethyl 2-((2-allyl-4-fluorophenyl)amino)-4-(trifluoromethyl)benzoate 5b

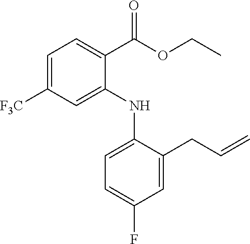 [SMILES: C=CCc1cc(F)ccc1Nc1cc(C(F)(F)F)ccc1C(=O)OCC]

To a solution of ethyl 2-((2-bromo-4-fluorophenyl)amino)-4-(trifluoromethyl)benzoate (2.50 g, 6.15 mmol) in DMF (30 mL), under N₂, was added allyltributylstannane (2.29 mL, 7.39 mmol) and Pd(PPh₃)₄ (0.213 g, 0.185 mmol) and the reaction mixture was stirred to 80° C. for 16 h. The reaction mixture was allowed to cool to ambient temperature, quenched with ice-water (200 mL), extracted with EtOAc (2×100 mL), and the combined organic extracts washed with H₂O (100 mL), brine, dried over Na₂SO₄, filtered, the solvent evaporated under reduced pressure. The crude residue was dissolved in DCM, absorbed onto silica gel and purified by silica gel flash column chromatography (100 g) eluting with 5% EtOAc-pet ether. Pure product fractions were combined and evaporated under reduced pressure, and the solid freeze dried to afford ethyl 2-((2-allyl-4-fluorophenyl)amino)-4-(trifluoromethyl)benzoate 5b (2.45 g, 85% yield) as a yellow oil, in 65% purity. HPLC/MS 3.23 min (F), [M+H]⁺ 368.0.

### Step C: 2-((2-Allyl-4-fluorophenyl)amino)-4-(trifluoromethyl)benzoic acid 5c

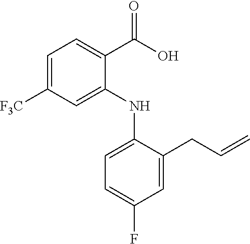 [SMILES: C=CCc1cc(F)ccc1Nc1cc(C(F)(F)F)ccc1C(=O)O]

To a solution of ethyl 2-((2-allyl-4-fluorophenyl)amino)-4-(trifluoromethyl)benzoate (2.45 g, 6.67 mmol) in THF (18 mL) was added LiOH (1.68 g, 40.0 mmol) dissolved in H₂O (6 mL), under N₂, and the reaction mixture was stirred at 60° C. for 3 h. The reaction mixture was allowed to cool to ambient temperature and evaporated under reduced pressure. The residue was dissolved in H₂O (5 mL), acidified with 1.5 N HCl to pH4, and the aqueous layer extracted with EtOAc (3×100 mL), the combined extracts dried over Na₂SO₄, filtered, the solvent evaporated under reduced pressure to afford 2-((2-allyl-4-fluorophenyl)amino)-4-(trifluoromethyl)benzoic acid 5c (2.30 g, 83% yield) as a yellow solid, in 82% purity. HPLC/MS 2.76 min (F), [M+H]⁺ 338.2.

### Step D: 2-((2-Allyl-4-fluorophenyl)amino)-N-(2-allyl-6-methoxypyridin-3-yl)-4-(trifluoromethyl)benzoic acid 5d

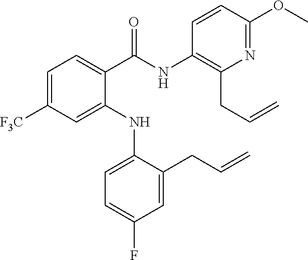 [SMILES: C=CCc1cc(F)ccc1Nc1cc(C(F)(F)F)ccc1C(=O)Nc1ccc(OC)nc1CC=C]

To a solution of 2-((2-allyl-4-fluorophenyl)amino)-4-(trifluoromethyl)benzoic acid (2.30 g, 6.78 mmol) in DMF (25 mL) was added DIEA (3.55 mL, 20.3 mmol) and HATU (3.87 g, 10.2 mmol), under N₂, and the reaction mixture was stirred for 20 min, at which time Int-1a (1.34 g, 8.13 mmol) dissolved in DMF (5 mL) was added, dropwise, and the reaction mixture was stirred at room temperature for 2 h. The reaction mixture was quenched with ice-water (200 mL), extracted with EtOAc (2×200 mL), the combined organic extracts washed with H₂O (200 mL), brine, dried over Na₂SO₄, filtered, and the solvent evaporated under reduced pressure. The crude residue was dissolved in DCM, absorbed onto silica gel and purified by silica gel flash column chromatography (75 g) eluting with 20% EtOAc-pet ether. Pure product fractions were combined and evaporated under reduced pressure to afford 2-((2-allyl-4-fluorophenyl)amino)-N-(2-allyl-6-methoxypyridin-3-yl)-4-(trifluoromethyl)benzoic acid 5d (2.50 g, 69% yield) as a yellow solid. HPLC/MS 3.11 min (F), [M+H]⁺ 486.2. ¹H NMR (400 MHz, DMSO-d₆) δ 10.2 (2, 1H), 9.27 (s, 1H), 8.00-8.05 (d, 1H), 7.73-7.68 (d, 1H), 7.34-7.82 (m, 1H), 7.12-7.19 (m, 3H), 6.88 (s, 1H), 6.73-6.75 (d, 1H), 5.92-6.08 (m, 1H), 5.80-5.90 (m, 1H), 4.93-5.08 (m, 4H), 3.86 (s, 3H), 3.49-3.51 (m, 2H), 3.28-3.32 (m, 2H).

### Step E: 1-(2-Allyl-4-fluorophenyl)-3-(2-allyl-6-methoxy-pyridin-3-yl)-7-(trifluoromethyl)-2,3-dihydroqiuinazolin-4(11H)-one 5e

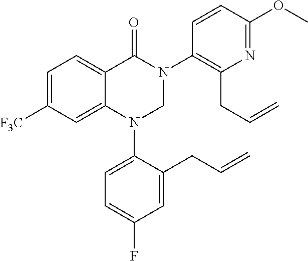 [SMILES: C=CCc1cc(F)ccc1N1CN(c2ccc(OC)nc2CC=C)C(=O)c2ccc(C(F)(F)F)cc21]

To a solution of 2-((2-allyl-4-fluorophenyl)amino)-N-(2-allyl-6-methoxypyridin-3-yl)-4-(trifluoromethyl)benzamide (1.50 g, 3.09 mmol) in MeCN (20 mL), under N₂, was added Cs₂CO₃ (4.03 g, 12.4 mmol) and diiodomethane (0.748 mL, 9.27 mmol), drop-wise, and the reaction mixture was stirred at 80° C. for 4 h. The reaction mixture was allowed to cool to ambient temperature, quenched with ice-water (100 mL), extracted with EtOAc (2×200 mL), and the combined organic extracts washed with H₂O (100 mL), brine, dried over Na₂SO₄, filtered, the solvent evaporated under reduced pressure. The crude residue was dissolved in DCM, absorbed onto silica gel and purified by silica gel flash column chromatography (50 g) eluting with 20% EtOAc-pet ether. Pure product fractions were combined and evaporated under reduced pressure. The isolated material was further purified by semi-prep HPLC (XSELECT C_{18, 19×150} mm, 5 μm) eluting with a MeCN—H₂O gradient. Product fractions were combined and evaporated under reduced pressure to afford 1-(2-allyl-4-fluorophenyl)-3-(2-allyl-6-methoxy-pyridin-3-yl)-7-(trifluoromethyl)_{2,3}-dihydroquinazolin-4(1H)-one 5e (110 mg, 7% yield) as a yellow semi-solid. HPLC/MS 3.05 min (F), [M+H]⁺ 498.1. ¹H NMR (400 MHz, DMSO-d₆) δ 8.07-8.09 (d, 1H), 7.67-7.69 (m, 1H), 7.44-7.46 (m, 1H), 7.25-7.29 (m, 3H), 6.76-6.78 (d, 1H), 6.32-6.44 (m, 1H), 5.76-6.10 (m, 2H), 5.63-5.65 (d, 0.5H), 5.13-5.18 (m, 1H), 4.92-5.00 (m, 4H), 4.72-4.75 (d, 0.5H), 3.85 (s, 3H), 3.35-3.40 (m, 4H).

### Step F: 8-Fluoro-15-methoxy-3-(trifluoromethyl)-10,13-dihydro-19H-5,18-methanodibenzo[b,l]pyrido[3,2-f][1,5]diazacyclotridecin-19-one 5f

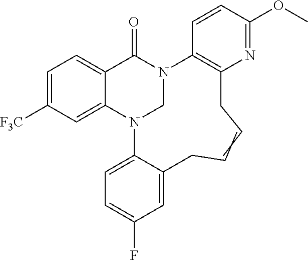 [SMILES: COc1ccc2c(n1)CC=CCc1cc(F)ccc1N1CN2C(=O)c2ccc(C(F)(F)F)cc21]

A 100-mL, sealed tube fitted with a magnetic stir-bar was charged with 1-(2-allyl-4-fluorophenyl)-3-(2-allyl-6-methoxypyridin-3-yl)-7-(trifluoromethyl)-2,3-dihydroquinazolin-4(1H)-one (150 mg, 0.302 mmol). DCE (35 mL) and Hoveyda-Grubbsii (51.2 mg, 0.060 mmol) were added under N₂ atmosphere and the light brown reaction mixture was heated at 80° C. for 16 h. The reaction mixture was allowed to cool to ambient temperature and filtered over celite, washed with DCM (100 mL) and the solvent evaporated under reduced pressure. The crude residue was dissolved in DCM, absorbed onto silica gel and purified by silica gel flash column chromatography (25 g) eluting with 20% EtOAc-pet ether. Pure product fractions were combined and evaporated under reduced pressure to afford 8-fluoro-15-methoxy-3-(trifluoromethyl)-10,13-dihydro-19H-5,18-methanodibenzo[b,l]pyrido[3,2-f][1,5]diazacyclotridecine-19-one 5f (75 mg, 52% yield) as an off-white solid. HPLC/MS 2.90 min (F), [M+H]⁺ 470.2. ¹H NMR (DMSO-d₆, 400 MHz) δ 8.09-8.11 (d, 1H), 7.70-7.72 (d, 1H), 7.51-7.58 (m, 2H), 7.24-7.29 (m, 2H), 6.77-6.79 (d, 1H), 6.53 (s, 1H), 5.48-5.51 (m, 2H), 5.35-5.38 (d, 1H), 4.19-4.33 (m, 3H), 3.89 (s, 3H), 3.26-3.15 (m, 2H).

### Step G: 8-Fluoro-3-(trifluoromethyl)-10,13-dihydro-19H-5,18-methanodibenzo[b,l]pyrido[3,2 f][1,5]diaza cyclotridecin-15,19(14H)-dione Example 5 and 8-fluoro-1¹-iodo-3-(trifluoromethyl)-10,11,12,13-tetrahydro-19H-5,18-methanodibenzo[b,l]pyrido[3,2-f][1,5]diazacyclotridecine-15,19(14H)-dione OR 8-fluoro-1²-iodo-3-(trifluoromethyl)-10,11,12,13-tetrahydro-19H-5,18-methanodibenzo[b,l]pyrido[3,2-f][1,5]diazacyclotridecine-15,19(14H)-dione 6a

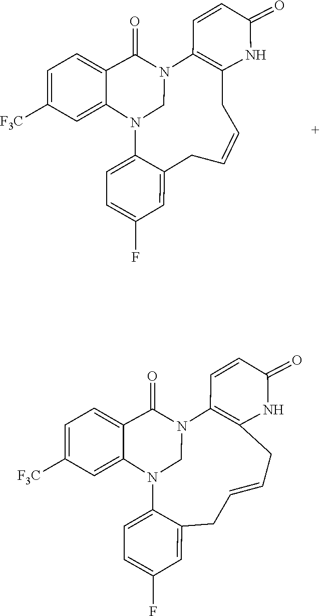 [SMILES: O=C1c2ccc(C(F)(F)F)cc2N2CN1c1ccc(=O)[nH]c1C/C=C\Cc1cc(F)ccc12 ; O=C1c2ccc(C(F)(F)F)cc2N2CN1c1ccc(=O)[nH]c1C/C=C/Cc1cc(F)ccc12]

To a solution of 8-fluoro-15-methoxy-3-(trifluoromethyl)-10,13-dihydro-19H-5,18-methanodibenzo[b,l]pyrido[3,2-f][1,5]diazacyclotridecin-19-one (0.075 g, 0.160 mmol) in MeCN (3 mL), cooled to 0° C. under N₂, was added iodotrimethylsilane (0.065 mL, 0.479 mmol), drop-wise, and the reaction mixture was stirred at 80° C. for 1 h. The reaction mixture was concentrated under reduced pressure, the residue quenched with sat'd aq. Na₂S₂O₃ (5 mL) and extracted with EtOAc (3×10 mL). The combined organic extracts were washed with H₂O (10 mL), brine, dried over Na₂SO₄, filtered, the solvent evaporated under reduced pressure. The crude residue was purified by semi-prep HPLC (XSELECT, C_{18, 10×250} mm, 5 um), eluting with a 70% H₂O-MeCN (0.1% HCO₂H) to 100% MeCN (0.1% HCO₂H) gradient. Pure product fractions were combined and freeze dried to afford 8-fluoro-3-(trifluoromethyl)-10,13-dihydro-19H-5,18-methanodibenzo[b,l]pyrido[3,2f][1,5]diazacyclotridecin-15,19(14H)-dione (13 mg, 18% yield) as an off white solid. HPLC/MS 2.19 min (F), [M+H]⁺ 456.0. ¹H NMR (DMSO-d₆, 400 MHz) δ 12.04 (brs, 1H), 8.09 (d, J=8.4 Hz, 1H), 7.60-7.49 (m, 2H), 7.45 (d, J=9.4 Hz, 1H), 7.32-7.14 (m, 2H), 6.52 (s, 1H), 6.26 (d, J=9.1 Hz, 1H), 5.58-5.39 (m, 2H), 5.32 (d, J=10.0 Hz, 1H), 4.27-4.11 (m, 2H), 4.03 (t, J=12.1 Hz, 1H), 3.14-3.03 (m, 1H), 2.98-2.89 (m, 1H). Fractions from a second peak were combined and freeze dried to afford either 8-fluoro-1¹-iodo-3-(trifluoromethyl)-10,11,12,13-tetrahydro-19H-5,18-methanodibenzo[b,l]pyrido[3,2-f][1,5]diazacyclotridecine-15,19(14H)-dione OR 8-fluoro-1²-iodo-3-(trifluoromethyl)-10,11,12,13-tetrahydro-19H-5,18-methanodibenzo[b,l]pyrido[3,2-J][1,5]diazacyclotridecine-15,19(14H)-dione 6a (20 mg, 6% yield). HPLC/MS 2.19 min (F), [M+H]⁺ 584.0, in 58% purity.

### Step H: 8-Fluoro-3-(trifluoromethyl)-10,11,12,13-tetrahydro-19H-5,18-methanodibenzo[b,l]pyridino [3,2-f][1,5]diazacyclotridecine-15,19(14H)-dione Example 6

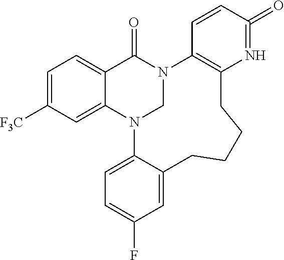

To a solution of 8-fluoro-1-iodo-3-(trifluoromethyl)-10,11,12,13-tetrahydro-19H-5,18-methanodibenzo[b,l]pyrido[3,2-f][1,5]diazacyclotridecine-15,19(14H)-dione OR 8-fluoro-1²-iodo-3-(trifluoromethyl)-10,11,12,13-tetrahydro-19H-5,18-methanodibenzo[b,l]pyrido[3,2-f][1,5]diazacyclotridecine-15,19(14H)-dione (25 mg, 0.043 mmol) in DMF (1 mL) was added DBU (6.46 μl, 0.043 mmol) and the reaction mixture was stirred at room temperature for 16 h then 60° C. for 6 h. The reaction mixture was allowed to cool to ambient temperature, quenched with ice-water (10 mL), extracted with EtOAc (2×10 mL), and the combined organic extracts washed with H₂O (10 mL), brine, dried over Na₂SO₄, filtered and the solvent evaporated under reduced pressure to afford a white solid. The solid was dissolved in MeOH (3 mL), purged with N₂, to which was added 20% Pd(OH)₂ (9.25 mg, 0.013 mmol) and the reaction mixture stirred under H₂ atmosphere for 16 h. The reaction mixture was filtered over celite, washed with MeOH (10 mL) and the solvent evaporated under reduced pressure. The crude residue was purified by semi-prep HPLC (X-BRIDGE C_{18, 19×150} mm, 5 μm) eluting with a 95% MeCN—H₂O to 100% MeCN gradient. The product fractions were combined and evaporated under reduced pressure then dried under vacuo using Genevac to afford 8-fluoro-3-(trifluoromethyl)-10,11,12,13-tetrahydro-19H-5,18-methanodibenzo[b,l]pyridino[3,2-f][1,5]diazacyclotridecine-15,19(14H)-dione (4.5 mg, 22% yield) as an off-white solid. HPLC/MS 3.12 min (F), [M+H]⁺ 457.7. ¹H NMR (DMSO-d₆, 400 MHz) δ 11.84 (brs, 1H), 8.08 (d, J=8.0 Hz, 1H), 7.48 (dd, J=8.8, 5.5 Hz, 1H), 7.43 (d, J=9.5 Hz, 1H), 7.36 (dd, J=9.6, 3.0 Hz, 1H), 7.26-7.20 (m, 2H), 6.40 (s, 1H), 6.21 (d, J=9.6 Hz, 1H), 5.57 (d, J=11.6 Hz, 1H), 4.69 (d, J=11.5 Hz, 1H), 2.75-2.64 (m, 2H), 2.42-2.35 (m, 1H), 2.29-2.20 (m, 1H), 1.71-1.58 (m, 2H), 1.50-1.35 (m, 2H).

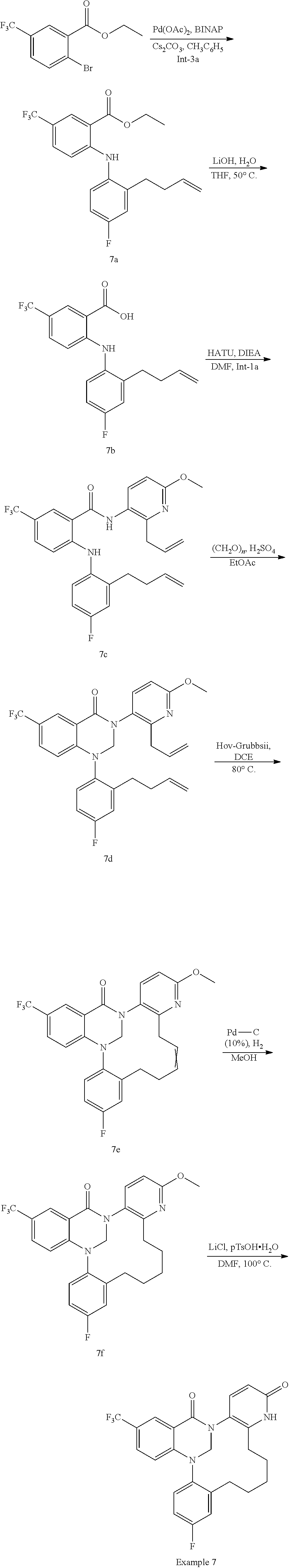

### Example 7

### 3⁴-Fluoro-2⁶-(trifluoromethyl)-1¹,1²,2¹,2²,2³,2⁴-hexahydro-2(3,1)-quinazolina-1(5,6)-pyridina-3(1,2)-benzenacyclooctaphane-1²,2⁴-dione

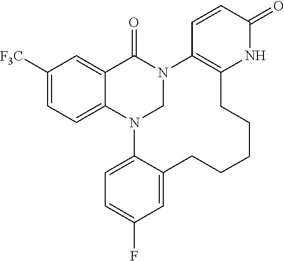 [SMILES: O=C1c2cc(C(F)(F)F)ccc2N2CN1c1ccc(=O)[nH]c1CCCCCc1cc(F)ccc12]

### Step A: Ethyl 2-((2-(but-3-en-1-yl)-4-fluorophenyl)amino)-5-(trifluoromethyl)benzoate 7a

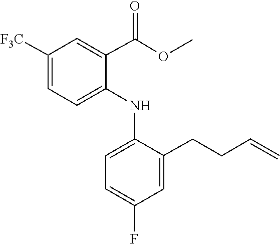 [SMILES: C=CCCc1cc(F)ccc1Nc1ccc(C(F)(F)F)cc1C(=O)OC]

To ethyl 2-bromo-5-(trifluoromethyl)benzoate (8.00 g, 26.9 mmol) and 2-(but-3-en-1-yl)-4-fluoroaniline Int-3a (5.34 g, 32.3 mmol), was added N₂ purged toluene (80 mL), followed by palladium(II) acetate (0.363 g, 1.62 mmol) and BINAP (1.61 g, 2.59 mmol), the reaction mixture purged further with N₂, to which was added Cs₂CO₃ (12.3 g, 37.7 mmol), and the reaction mixture was heated at 80° C. for 24 h. The reaction mixture was cooled to ambient temperature, diluted with EtOAc, quenched and washed with H₂O (2×), brine, dried over Na₂SO₄, filtered and the solvent evaporated under reduced pressure. The crude residue was dissolved in DCM, preabsorbed onto a silica gel prepacked column and purified by silica gel flash column chromatography (330 g) eluting with a 100% hexanes to 60% EtOAc-hexanes gradient. Product fractions were combined and evaporated under reduced pressure to afford ethyl 2-((2-(but-3-en-1-yl)-4-fluorophenyl)amino)-5-(trifluoromethyl)benzoate 7a (5.55 g, 51% yield) as a light yellow oil. HPLC/MS 1.61 min (A), [M+H]⁺ 382.1. ¹H NMR (DMSO-d₆, 400 MHz) δ 9.3-9.6 (m, 1H), 8.13 (d, 1H, J=2.0 Hz), 7.6-7.7 (m, 1H), 7.3-7.4 (m, 1H), 7.2-7.3 (m, 1H), 7.14 (d, 1H, J=2.9 Hz), 6.68 (d, 1H, J=8.8 Hz), 5.7-5.8 (m, 1H), 4.90 (s, 2H), 4.38 (d, 2H, J=6.8 Hz), 2.62 (s, 2H), 2.2-2.3 (m, 2H), 1.37 (t, 3H, J=7.1 Hz).

### Step B: 2-(But-3-en-1-yl)-4-fluorophenyl)amino)-5-(trifluoromethyl)benzoic acid 7b

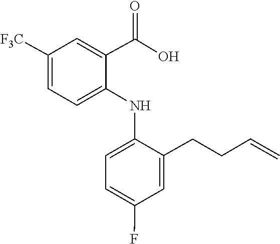 [SMILES: C=CCCc1cc(F)ccc1Nc1ccc(C(F)(F)F)cc1C(=O)O]

To ethyl 2-((2-(but-3-en-1-yl)-4-fluorophenyl)amino)-5-(trifluoromethyl)benzoate (5.55 g, 14.6 mmol), was added THF (75 mL) and H₂O (25 mL), followed by LiOH (1.39 g, 58.2 mmol), and the reaction mixture was stirred at 50° C. for 24 h. The reaction mixture was allowed to cool to ambient temperature, acidified with 1N HCl to pH5, extracted with EtOAc (2×), and the organic extracts combined, washed with H₂O, brine, dried over Na₂SO₄, filtered and the solvent evaporated under reduced pressure to afford 2-(but-3-en-1-yl)-4-fluorophenyl)amino)-5-(trifluoromethyl)benzoic acid 7b (5.34 g, 100% yield) as a beige solid. HPLC/MS 1.54 min (A), [M+H]⁺ 354.0. ¹H NMR (DMSO-d₆, 400 MHz) δ 13.58 (br s, 1H), 9.74 (s, 1H), 8.13 (d, 1H, J=1.5 Hz), 7.61 (dd, 1H, J=2.2, 9.0 Hz), 7.35 (dd, 1H, J=5.6, 8.6 Hz), 7.26 (dd, 1H, J=2.9, 9.8 Hz), 7.1-7.2 (m, 1H), 6.71 (d, 1H, J=8.8 Hz), 5.7-5.9 (m, 1H), 4.6-5.1 (m, 2H), 2.6-2.7 (m, 2H), 2.2-2.3 (m, 2H).

### Step C: N-(2-Allyl-6-methoxypyridin-3-yl)-2-((2-(but-3-en-1-yl)-4-fluorophenyl)amino)-5-(trifluoromethyl) benzamide 7c

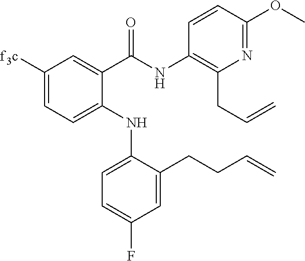

To 2-((2-(but-3-en-1-yl)-4-fluorophenyl)amino)-5-(trifluoromethyl)benzoic acid (1.10 g, 3.11 mmol), and 2-allyl-6-methoxypyridin-3-amine Int-1a (0.613 g, 3.74 mmol), in DMF (20 mL) was added HATU (1.54 g, 4.05 mmol), followed by TEA (1.30 mL, 9.34 mmol) and the reaction mixture was stirred at ambient temperature for 18 h. The reaction mixture was diluted with EtOAc, washed with H₂O (3×), brine, dried over Na₂SO₄, filtered and the solvent evaporated under reduced pressure. The crude residue was dissolved in DCM, absorbed onto a silica gel packed precolumn and purified by silica gel flash column chromatography (120 g) eluting with a 100% heptane to 100% EtOAc gradient. Pure product fractions were combined and evaporated under reduced pressure to afford N-(2-allyl-6-methoxypyridin-3-yl)-2-((2-(but-3-en-1-yl)-4-fluorophneyl)amino)-5-(trifluoromethyl)benzamide 7c (1.24 g, 77% yield) as a yellow oil. HPLC/MS 1.55 min (A), [M+H]⁺ 500.1. ¹H NMR (DMSO-d₆, 400 MHz) δ 10.24 (s, 1H), 9.74 (s, 1H), 8.22 (s, 1H), 7.65 (d, 1H, J=8.8 Hz), 7.6-7.6 (m, 1H), 7.3-7.4 (m, 1H), 7.22 (br d, 1H, J=9.8 Hz), 7.11 (br d, 1H, J=3.4 Hz), 6.75 (d, 2H, J=8.8 Hz), 6.04 (s, 1H), 5.7-5.8 (m, 1H), 5.04 (s, 2H), 4.9-5.0 (m, 2H), 3.87 (s, 3H), 3.5-3.5 (m, 2H), 2.6-2.7 (m, 2H), 2.2-2.3 (m, 2H).

### Step D: 3-(2-Allyl-6-methoxypyridin-3-yl)-1-(2-(but-3-en-1-yl)-4-fluorophneyl)-6-(trifluoromethyl)-2,3-dihydroquinazolin-4(11H)-one 7d

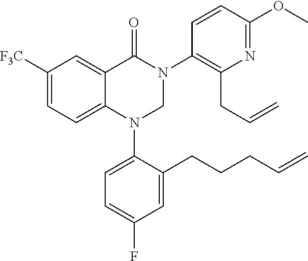 [SMILES: C=CCCCc1cc(F)ccc1N1CN(c2ccc(OC)nc2CC=C)C(=O)c2cc(C(F)(F)F)ccc21]

To a solution of N-(2-allyl-6-methoxypyridin-3-yl)-2-((2-(but-3-en-1-yl)-4-fluorophenyl)amino)-5-(trifluoromethyl)benzamide (1.24 g, 2.48 mmol), in EtOAc (100 mL), was added paraformaldehyde (1.49 g, 49.6 mmol), followed by conc. H₂SO₄ (0.662 mL, 12.4 mmol), dropwise, and the reaction mixture was stirred at ambient temperature for 4.5 h. The reaction mixture was diluted with EtOAc, quenched and washed with 10% NaHCO₃ (2×), H₂O, brine, dried over Na₂SO₄, filtered and the solvent evaporated under reduced pressure. The crude residue was dissolved in DCM, absorbed onto a silica gel packed precolumn and purified by silica gel flash column chromatography (120 g) eluting with a 100% heptane to 40% EtOAc gradient. Pure product fractions were combined and evaporated under reduced pressure to afford 3-(2-allyl-6-methoxypyridin-3-yl)-1-(2-(but-3-en-1-yl)-4-fluorophneyl)-6-(trifluoromethyl)-2,3-dihydroquinazolin-4(1H)-one 7d (772.3 mg, 59% yield) as a viscous oil. HPLC/MS 1.49 min (A, [M+H]⁺ 512.2. ¹H NMR (DMSO-d₆, 400 MHz) δ 8.10 (s, 1H), 7.6-7.7 (m, 2H), 7.3-7.5 (m, 2H), 7.23 (br dd, 1H, J=2.7, 8.6 Hz), 6.78 (d, 1H, J=8.8 Hz), 6.3-6.5 (m, 1H), 5.9-6.1 (m, 1H), 5.6-5.9 (m, 2H), 5.3-5.3 (m, 1H), 4.9-5.1 (m, 4H), 4.6-4.8 (m, 1H), 3.86 (s, 3H), 3.4-3.5 (m, 2H), 2.6-2.8 (m, 2H), 2.3-2.4 (m, 2H).

### Step E: 3⁴-Fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-6-en-2⁴-one 7e

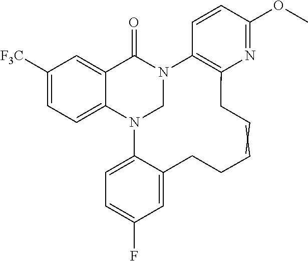 [SMILES: COc1ccc2c(n1)CC=CCCc1cc(F)ccc1N1CN2C(=O)c2cc(C(F)(F)F)ccc21]

To a solution of 3-(2-allyl-6-methoxypyridin-3-yl)-1-(2-(but-3-en-1-yl)-4-fluorophenyl)-6-(trifluoromethyl)-2,3-dihydroquinazolin-4(1H)-one (770 mg, 1.51 mmol), in DCE (30 mL), purged with N₂, was added Hoveyda-Grubbsii (256 mg, 0.301 mmol), and the reaction mixture was stirred at 80° C. for 18 h. The reaction mixture was cooled to ambient temperature and the solvent evaporated under reduced pressure. The crude residue was dissolved in DCM, absorbed onto a silica gel packed precolumn and purified by silica gel flash column chromatography (80 g) eluting with a 100% heptane to 30% EtOAc gradient. Product fractions were combined and evaporated under reduced pressure to afford 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-6-en-2⁴-one 7e (506.6 mg, 67% yield) as a white solid. HPLC/MS 1.38 min (A), [M+H]⁺ 484.1. ¹H NMR (DMSO-d₆, 400 MHz) δ 8.0-8.2 (m, 1H), 7.6-7.8 (m, 2H), 7.2-7.4 (m, 3H), 6.80 (d, 1H, J=8.3 Hz), 6.3-6.5 (m, 1H), 5.7-6.0 (m, 1H), 5.1-5.6 (m, 2H), 4.36 (d, 1H, J=8.8 Hz), 3.87 (d, 3H, J=2.0 Hz), 3.4-3.8 (m, 1H), 3.25 (br s, 1H), 2.76 (s, 2H), 2.4-2.5 (m, 1H), 1.8-2.3 (m, 1H).

### Step F: 3⁴-Fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-2⁴-one 7f

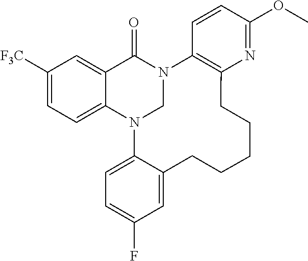 [SMILES: COc1ccc2c(n1)CCCCCc1cc(F)ccc1N1CN2C(=O)c2cc(C(F)(F)F)ccc21]

To a solution of 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-6-en-2⁴-one (501 mg, 1.04 mmol), in DCE (10.0 mL) and MeOH (1.0 mL), purged with N₂, was added Hoveyda-Grubbsii (44.0 mg, 0.052 mmol), followed by NaBH₄ (78 mg, 2.07 mmol), and the reaction mixture was stirred at ambient temperature for 22 h. The reaction mixture was quenched with H₂O, diluted with DCM, the layers partitioned, the organic phase dried over Na₂SO₄, filtered and the solvent evaporated under reduced pressure. The crude residue was dissolved in DCM, absorbed onto a silica gel packed precolumn and purified by silica gel flash column chromatography (120 g) eluting with a 100% heptane to 40% EtOAc gradient. Product fractions were combined and evaporated under reduced pressure to afford a white solid, consisting of 10% starting olefin. The solid was dissolved in EtOAc/EtOH (18:12 mL), the reaction purged with N₂ (3×), 10% Pd—C(27 mg; 0.257 mmol) was added, the reaction mixture purged with N₂, a balloon with hydrogen added, the reaction mixture sparged (3×) and allowed to stir at room temperature for 23 h. The reaction mixture was purged with N₂, filtered through a pad of celite, the celite washed with EtOAc, the solvent evaporated under reduced pressure and dried under vacuo to afford 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-2⁴-one 7f (1.21 g, 81% yield) as a white solid. HPLC/MS 1.39 min (A), [M+H]⁺ 486.2. ¹H NMR (DMSO-d₆, 400 MHz) δ 8.1-8.2 (m, 1H), 7.66 (d, 2H, J=8.3 Hz), 7.4-7.4 (m, 1H), 7.28 (br d, 1H, J=9.8 Hz), 7.2-7.3 (m, 1H), 6.74 (d, 1H, J=8.3 Hz), 6.43 (d, 1H, J=8.3 Hz), 5.81 (d, 1H, J=10.8 Hz), 4.76 (d, 1H, J=10.8 Hz), 3.84 (s, 3H), 2.6-2.8 (m, 1H), 2.5-2.6 (m, 2H), 2.4-2.5 (m, 1H), 1.7-1.9 (m, 2H), 1.4-1.6 (m, 2H), 1.1-1.3 (m, 2H).

### Step G: 3⁴-Fluoro-2⁶-(trifluoromethyl)-1¹,1²,2¹,2²,2³,2⁴-hexahydro-2(3,1)-quinazolina-1(5,6)-pyridina-3(1,2)-benzenacyclooctaphane-1²,2⁴-dione Example 7

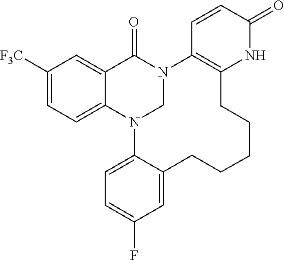 [SMILES: O=C1c2cc(C(F)(F)F)ccc2N2CN1c1ccc(=O)[nH]c1CCCCCc1cc(F)ccc12]

To a solution of 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclooctaphan-2⁴-one (1.42 g, 2.49 mmol), in DMF (25 mL), was added pTsOH (2.84 g, 14.9 mmol) and LiCl (0.634 g, 14.9 mmol), and the reaction mixture was stirred at 100° C. for 1.5 h. The reaction mixture was cooled to ambient temperature, diluted with EtOAc and washed with H₂O (2×), the precipitate filtered, washed with EtOAc and dried under vacuo to afford 3⁴-fluoro-2⁶-(trifluoromethyl)-1¹,1²,1¹,2²,2³,2⁴-hexahydro-2(3,1)-quinazolina-1(5,6)-pyridina-3(1,2)-benzenacyclo octaphane-1²,2⁴-dione (416.7 mg, 36% yield) as a white solid. The clear organic phase was washed with brine, dried over Na₂SO₄, filtered and the solvent evaporated under reduced pressure. The solid was suspended in diethyl ether, triturated, filtered and the solid washed with diethyl ether to afford additional 3⁴-fluoro-2⁶-(trifluoromethyl)-1¹,1²,2¹,2²,2³,2⁴-hexahydro-2(3,1)-quinazolina-1(5,6)-pyridina-3(1,2)-benzenacyclooctaphane-1²,2⁴-dione (695.5 mg, 59% yield) as a white solid. HPLC/MS 1.08 min (A), [M+H]⁺ 472.2. ¹H NMR (DMSO-d₆, 400 MHz) δ 11.4-11.9 (m, 1H), 8.12 (d, 1H, J=2.0 Hz), 7.68 (dd, 1H, J=2.0, 8.8 Hz), 7.3-7.5 (m, 2H), 7.2-7.3 (m, 2H), 6.41 (d, 1H, J=8.3 Hz), 6.23 (br d, 1H, J=9.3 Hz), 5.73 (d, 1H, J=10.8 Hz), 4.76 (d, 1H, J=11.2 Hz), 2.40 (br s, 2H), 1.80 (br d, 1H, J=7.3 Hz), 1.6-1.7 (m, 1H), 1.3-1.5 (m, 2H), 1.1-1.3 (m, 3H).

### Example 8

### 3⁴-Fluoro-2⁷-(trifluoromethyl)-1¹,1²,2¹,2²,2³,2⁴-hexahydro-2(3.1)-quinazolina-1(5,6)-pyridina-3(1,2)-benzenacyclooctaphane-1²,2⁴-dione

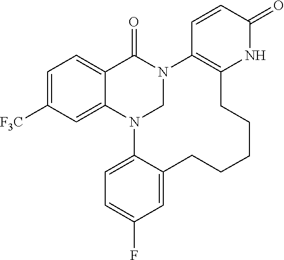 [SMILES: O=C1c2ccc(C(F)(F)F)cc2N2CN1c1ccc(=O)[nH]c1CCCCCc1cc(F)ccc12]

### Step A: Ethyl 2-((2-(allyl-4-fluorophenyl)amino)-4-(trifluoromethyl)benzoate 8a

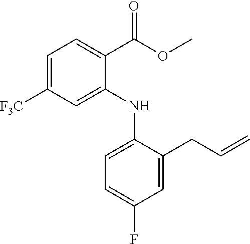 [SMILES: C=CCc1cc(F)ccc1Nc1cc(C(F)(F)F)ccc1C(=O)OC]

Following the procedure outlined in Example 7, Step A, substituting Int-2 with Int-2a and Int-3a with Int-3b, and heating the reaction mixture at 80° C. for 4 h, ethyl 2-((2-(allyl-4-fluorophenyl)amino)-4-(trifluoromethyl)benzoate 8a (3.87 g, 59% yield) was prepared as a light yellow oil. HPLC/MS 1.56 min (A), [M+H]⁺ 368.1. ¹H NMR (CD₃OD, 400 MHz) δ 9.2-9.5 (m, 1H), 8.13 (d, 1H, J=8.3 Hz), 7.3-7.3 (m, 1H), 7.0-7.2 (m, 2H), 6.94 (d, 1H, J=8.3 Hz), 6.81 (d, 1H, J=1.0 Hz), 5.8-6.0 (m, 1H), 4.9-5.1 (m, 2H), 4.4-4.5 (m, 2H), 3.36 (s, 1H), 1.4-1.5 (m, 3H).

### Step B: 2-((2-Allyl-4-fluorophenyl)amino)-4-(trifluoromethyl)benzoic acid 8b

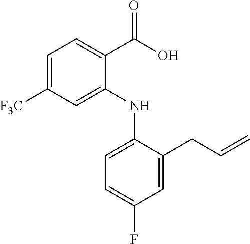 [SMILES: C=CCc1cc(F)ccc1Nc1cc(C(F)(F)F)ccc1C(=O)O]

Following the procedure outlined in Example 7, Step B, 2-((2-allyl-4-fluorophenyl)amino)-4-(trifluoromethyl)benzoic acid 8b (1.79 g, 95% yield) was prepared as an off-white solid. HPLC/MS 1.36 min (A), [M+H]⁺ 340.0. ¹H NMR (DMSO-d₆, 400 MHz) δ 12.6-14.3 (m, 1H), 9.62 (br s, 1H), 8.07 (d, 1H, J=7.8 Hz), 7.5-7.5 (m, 0.03H), 7.38 (dd, 1H, J=5.6, 8.6 Hz), 7.1-7.3 (m, 2H), 6.98 (dd, 1H, J=1.0, 8.3 Hz), 6.80 (d, 1H, J=1.0 Hz), 5.8-6.0 (m, 1H), 4.9-5.1 (m, 2H), 3.31 (d, 2H, J=6.4 Hz).

### Step C: 2-((2-Allyl-4-fluorophenyl)amino)-N-(2-but-en-1-yl)-6-methoxypyridin-3-yl)-4-(trifluoromethyl) benzamide 8c

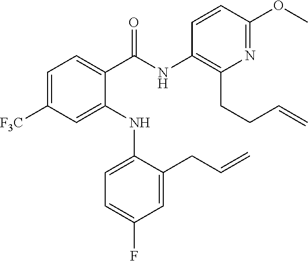 [SMILES: C=CCCc1nc(OC)ccc1NC(=O)c1ccc(C(F)(F)F)cc1Nc1ccc(F)cc1CC=C]

Following the procedure outlined in Example 7, Step C, substituting Int-1a with Int-1b, and stirring the reaction mixture at room temperature for 2 h, 2-((2-allyl-4-fluorophenyl)amino)-N-(2-but-en-1-yl)-6-methoxypyridin-3-yl)-4-(trifluoromethyl) benzamide 8c (1.11 g, 57% yield) was prepared as viscous yellow oil. HPLC/MS 1.52 min (A), [M+H]⁺ 500.0. ¹H NMR (DMSO-d₆, 400 MHz) δ 10.21 (s, 1H), 9.29 (s, 1H), 8.04 (d, 1H, J=8.3 Hz), 7.62 (d, 1H, J=8.3 Hz), 7.35 (dd, 1H, J=5.4, 8.3 Hz), 7.1-7.2 (m, 3H), 6.89 (d, 1H, J=1.5 Hz), 6.72 (d, 1H, J=8.3 Hz), 5.8-6.0 (m, 2H), 4.9-5.1 (m, 4H), 3.87 (s, 3H), 3.30 (d, 2H, J=6.4 Hz), 2.77 (dd, 2H, J=6.6, 9.0 Hz), 2.51 (br d, 1H, J=2.0 Hz), 2.4-2.5 (m, 1H).

### Step E: 1-(2-Allyl-4-fluorophenyl)-3-(2-(but-3-en-1-yl)-6-methoxypyridin-3-yl)-7-(trifluoromethyl)-2,3-dihydroquinazolin-4(1H)-one 8d

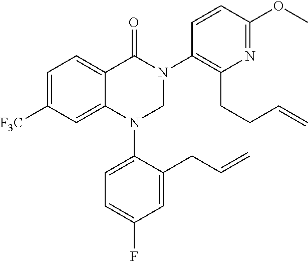 [SMILES: C=CCCc1nc(OC)ccc1N1CN(c2ccc(F)cc2CC=C)c2cc(C(F)(F)F)ccc2C1=O]

Following the procedure outlined in Example 7, Step D, stirring the reaction mixture at 65° C. for 0.5 h, 1-(2-allyl-4-fluorophenyl)-3-(2-(but-3-en-1-yl)-6-methoxypyridin-3-yl)-7-(trifluoromethyl)-2,3-dihydroquinazolin-4(1H)-one 8d (245 mg, 46% yield) was prepared as clear viscous oil. HPLC/MS 1.55 min (A), [M+H]⁺ 512.1. ¹H NMR (DMSO-d₆, 400 MHz) δ 8.0-8.2 (m, 1H), 7.6-7.7 (m, 1H), 7.4-7.5 (m, 1H), 7.2-7.3 (m, 3H), 6.75 (d, 1H, J=8.3 Hz), 6.47 (s, 1H), 6.32 (s, 1H), 6.3-6.5 (m, 1H), 5.76 (s, 2H), 5.6-5.7 (m, 1H), 5.2-5.3 (m, 1H), 4.8-5.2 (m, 6H), 4.6-4.7 (m, 1H), 3.87 (s, 3H), 3.38 (br d, 2H, J=6.4 Hz), 2.75 (br s, 2H), 2.3-2.5 (m, 2H).

### Step F: 3⁴-Fluoro-1⁶-methoxy-2⁷-(trifluoromethyl)-2¹,2²,2³,2⁴-tetrahydro-2(3,1)-quinazolina-1(3,2)-pyridina-3(1,2)-benzenacyclooctaphan-6-en-2⁴-one 8e

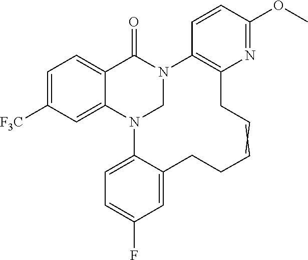 [SMILES: COc1ccc2c(n1)CC=CCCc1cc(F)ccc1N1CN2C(=O)c2ccc(C(F)(F)F)cc21]

Following the procedure outlined in Example 7, Step E, stirring the reaction mixture at 80° C. for 17 h, 3⁴-fluoro-1⁶-methoxy-2⁷-(trifluoromethyl)-2¹,2²,2³,2⁴-tetrahydro-2(3,1)-quinazolina-1(3,2)-pyridina-3(1,2)-benzenacyclooctaphan-6-en-2⁴-one 8e (98 mg, 41% yield) was prepared as a white solid. HPLC/MS 1.42 min (A), [M+H]⁺ 484.0. ¹H NMR (DMSO-d₆, 400 MHz) δ 8.10 (dd, 1H, J=7.8, 17.1 Hz), 7.6-7.7 (m, 1H), 7.4-7.5 (m, 1H), 7.2-7.4 (m, 2H), 7.18 (dd, 1H, J=1.2, 8.1 Hz), 6.6-6.8 (m, 1H), 6.43 (d, 1H, J=1.0 Hz), 6.29 (d, 1H, J=1.5 Hz), 6.3-6.4 (m, 1H), 5.91 (d, 1H, J=8.8 Hz), 5.6-5.9 (m, 1H), 5.68 (d, 1H, J=10.3 Hz), 5.2-5.5 (m, 2H), 4.3-4.6 (m, 1H), 3.86 (d, 3H, J=6.4 Hz), 3.61 (dd, 1H, J=10.5, 13.4 Hz), 3.1-3.3 (m, 1H), 2.89 (br s, 1H), 2.6-2.8 (m, 2H), 1.8-2.3 (m, 1H).

### Step G: 3⁴-Fluoro-1⁶-methoxy-2⁷-(trifluoromethyl)-2¹,2²,2³,2⁴-tetrahydro-2(3,1)-quinazolina-1(3,2)-pyridina-3(1,2)-benzenacyclooctaphan-2⁴-one 8f

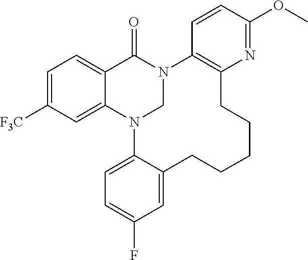 [SMILES: COc1ccc2c(n1)CCCCCc1cc(F)ccc1N1CN2C(=O)c2ccc(C(F)(F)F)cc21]

Following the procedure outlined in Example 7, Step F, substituting MeOH with EtOAc and stirring the reaction mixture for 18 h, then subjecting the reaction to additional 10% Pd—C(0.1 equiv) and stirring under H₂ atmosphere for an additional 30 h, 3⁴-fluoro-1⁶-methoxy-2⁷-(trifluoromethyl)-2¹,2²,2³,2⁴-tetrahydro-2(3,1)-quinazolina-1(3,2)-pyridina-3(1,2)-benzenacyclooctaphan-6-en-2⁴-one 8f (38 mg, 67% yield) was prepared as a white solid. HPLC/MS 1.48 min (B), [M+H]⁺ 486.3. ¹H NMR (CD₃OD, 400 MHz) δ 8.1-8.3 (m, 1H), 7.72 (d, 1H, J=8.3 Hz), 7.34 (d, 1H, J=5.4 Hz), 7.2-7.3 (m, 1H), 7.2-7.2 (m, 1H), 7.1-7.2 (m, 1H), 6.81 (d, 1H, J=8.8 Hz), 6.52 (s, 1H), 5.79 (d, 1H, J=10.8 Hz), 4.82 (d, 2H, J=10.8 Hz), 3.96 (s, 3H), 2.8-2.9 (m, 1H), 2.6-2.8 (m, 2H), 2.4-2.5 (m, 1H), 1.87 (dt, 2H, J=6.8, 13.9 Hz), 1.5-1.7 (m, 2H), 1.3-1.4 (m, 2H).

### Step H: 3⁴-Fluoro-2⁷-(trifluoromethyl)-1¹,1²,2¹,2²,2³,2⁴-hexahydro-2(3,1)-quinazolina-1(5,6)-pyridina-3(1,2)-benzenacyclooctaphane-1²,2⁴-dione Example 8

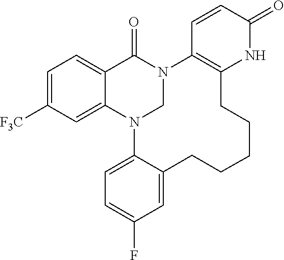 [SMILES: O=C1c2ccc(C(F)(F)F)cc2N2CN1c1ccc(=O)[nH]c1CCCCCc1cc(F)ccc12]

Following the procedure outlined in Example 7, Step E, stirring the reaction mixture at 100° C. for 2 h, 3⁴-fluoro-2⁷-(trifluoromethyl)-1¹,1²,2¹,2²,2³,2⁴-hexahydro-2(3,1)-quinazolina-1(5,6)-pyridina-3(1,2)-benzenacyclooctaphane-1²,2⁴-dione (37 mg, 66% yield) was prepared as a white solid. HPLC/MS 1.10 min (A), [M+H]⁺ 472.2. ¹H NMR (DMSO-d₆, 400 MHz) δ 11.4-12.1 (m, 1H), 8.10 (d, 1H, J=7.8 Hz), 7.4-7.5 (m, 2H), 7.1-7.3 (m, 3H), 6.40 (s, 1H), 6.23 (d, 1H, J=9.3 Hz), 5.72 (d, 1H, J=10.8 Hz), 4.75 (d, 1H, J=10.8 Hz), 2.3-2.5 (m, 4H), 1.6-1.9 (m, 2H), 1.3-1.6 (m, 2H), 1.1-1.3 (m, 2H).

### Example 9

3⁴-Fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphane-2⁴-one

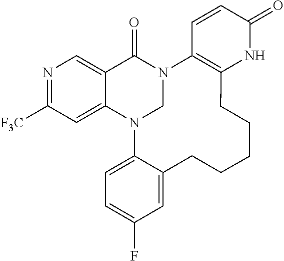 [SMILES: O=C1c2cnc(C(F)(F)F)cc2N2CN1c1ccc(=O)[nH]c1CCCCCc1cc(F)ccc12]

### Step A: Ethyl 4-((2-allyl-4-fluorophenyl)amino)-6-(trifluoromethyl)nicotinate 9a

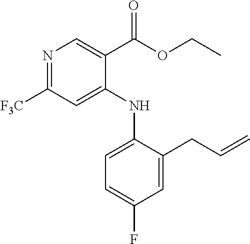 [SMILES: C=CCc1cc(F)ccc1Nc1cc(C(F)(F)F)ncc1C(=O)OCC]

To a solution of 2-allyl-4-fluoroaniline (1.74 g, 11.5 mmol) in EtOH (60 mL) was added ethyl 4-chloro-6-(trifluoromethyl)nicotinate (3.21 g, 12.7 mmol), followed by conc. HCl (2.5 mL, 11.5 mmol). The reaction was heated at 80° C. for 5 d. The reaction mixture was cooled to ambient temperature, quenched with sat'd aq. NaHCO₃ solution, and extracted with EtOAc (3×). The combined organic extracts were washed with brine, dried over MgSO₄, filtered, the solvent evaporated under reduced pressure and the crude product purified by silica gel flash column chromatography (120 g) eluting with a 100% heptanes to 20% EtOAc-hseptanes gradient. The product fractions were combined and evaporate under reduced pressure to afford ethyl 4-((2-allyl-4-fluorophenyl)amino)-6-(trifluoromethyl)nicotinate 9a (1.83 g, 30% yield) as a light yellow oil, in 70% purity. HPLC/MS 1.37 min (A), [M+H]⁺ 369.1.

### Step B: 4-((2-Allyl-4-fluorophenyl)amino)-6-(trifluoromethyl)nicotinic acid 9b

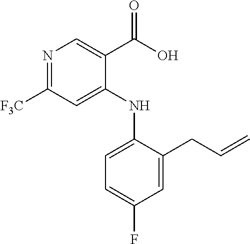 [SMILES: C=CCc1cc(F)ccc1Nc1cc(C(F)(F)F)ncc1C(=O)O]

Following the procedure outlined in Example 7, Step B, using a THF/MeOH/H₂O solvent system and heating the reaction mixture at 50° C. for 2 h, 4-((2-allyl-4-fluorophenyl)amino)-6-(trifluoromethyl) nicotinic acid 9b (875 mg, 67% yield) was prepared as an off-white solid. HPLC/MS 1.15 min (A), [M+H]⁺ 341.1. ¹H NMR (DMSO-d₆, 400 MHz) δ 13.9 (br s, 1H), 9.92 (s, 1H), 8.91 (s, 1H), 7.43 (dd, J=5.4, 8.3 Hz, 1H), 7.18-7.28 (m, 2H), 6.71 (s, 1H), 5.87 (tdd, J=6.5, 10.1, 16.9 Hz, 1H), 4.89-5.04 (m, 1H), 3.31 (d, J=6.4 Hz, 2H).

### Step C: 4-((2-Allyl-4-fluorophenyl)amino)-N-(2-(but-3-en-1-yl)-6-methoxypyridin-3-yl)-6-(trifluoromethyl)nicotinamide 9c

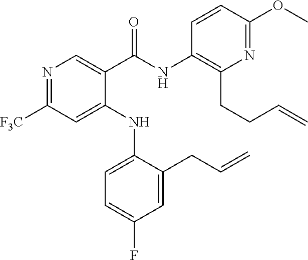 [SMILES: C=CCCc1nc(OC)ccc1NC(=O)c1cnc(C(F)(F)F)cc1Nc1ccc(F)cc1CC=C]

Following the procedure outlined in Example 7, Step C, substituting Int-1a with Int-1b, DMF with MeCN, and stirring the reaction mixture at room temperature for 20 h, 4-((2-allyl-4-fluorophenyl)amino)-N-(2-(but-3-en-1-yl)-6-methoxypyridin-3-yl)-6-(trifluoromethyl)nicotinamide 9c (985 mg, 74% yield) was prepared as a yellow foam, in 85% purity. HPLC/MS 1.41 min (A), [M+H]⁺ 501.1.

### Step D: 1-(2-Allyl-4-fluorophenyl)-3-(2-(but-3-en-1-yl)-6-methoxypyridin-3-yl)-7-(trifluoromethyl)-2,3-dihydropyrido[4,3-d]pyrimidin-4(11H)-one 9d

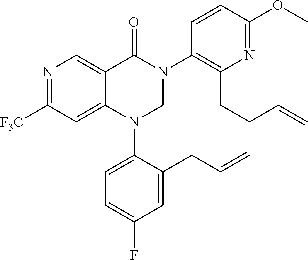 [SMILES: C=CCCc1nc(OC)ccc1N1CN(c2ccc(F)cc2CC=C)c2cc(C(F)(F)F)ncc2C1=O]

Following the procedure outlined in Example 1, Step C, stirring the reaction mixture at 80° C. for 23 h, 1-(2-allyl-4-fluorophenyl)-3-(2-(but-3-en-1-yl)-6-methoxypyridin-3-yl)-7-(trifluoromethyl)-2,3-dihydro pyrido[4,3-d]pyrimidin-4(1H)-one 9d (287 mg, 33% yield) was prepared as a yellow oil. HPLC/MS 1.39 min (A), [M+H]⁺ 513.2. ¹H NMR (CD₃OD, 400 MHz) δ 8.95 (s, 1H), 7.57-7.64 (m, 1H), 7.45-7.50 (m, 1H), 7.17-7.28 (m, 2H), 6.68-6.72 (m, 1H), 6.45-6.49 (m, 1H), 5.82-6.00 (m, 2H), 5.67-5.73 (m, 1H), 5.21-5.36 (m, 1H), 4.97-5.03 (m, 2H), 4.84-4.92 (m, 2H), 3.89-3.96 (m, 3H), 3.43-3.50 (m, 2H), 2.77-2.88 (m, 2H), 2.52-2.57 (m, 2H).

### Step E: 3⁴-Fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphan-5-en-2⁴-one 9e

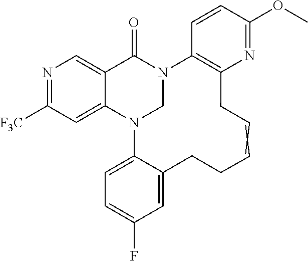 [SMILES: COc1ccc2c(n1)CC=CCCc1cc(F)ccc1N1CN2C(=O)c2cnc(C(F)(F)F)cc21]

Following the procedure outlined in Example 7, Step E, stirring the reaction mixture at 80° C. for 1 h, 3⁴-fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphan-5-en-2⁴-one 9e (99 mg, 66% yield) was prepared as a clear oil. HPLC/MS 1.22/1.25 min (A), [M+H]⁺ 485.1.

### Step F: 3⁴-Fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphan-2⁴-one 9f

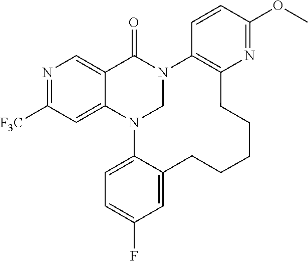 [SMILES: COc1ccc2c(n1)CCCCCc1cc(F)ccc1N1CN2C(=O)c2cnc(C(F)(F)F)cc21]

Following the procedure outlined in Example 7, Step F, substituting MeOH with EtOH and stirring the reaction mixture for 72 h, 3⁴-fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphane-2⁴-one 9f (73 mg, 85% yield) was prepared as a colorless oil. HPLC/MS 1.25 min (A), [M+H]⁺ 487.1. ¹H NMR (CD₃OD, 400 MHz) δ 9.02 (s, 1H), 7.60 (d, J=8.3 Hz, 1H), 7.38 (dd, J=5.1, 8.6 Hz, 1H), 7.11-7.22 (m, 2H), 6.69 (d, J=8.3 Hz, 1H), 6.57 (s, 1H), 5.85 (d, J=10.8 Hz, 1H), 4.84 (s, 1H), 3.90 (s, 3H), 2.79 (td, J=7.1, 14.2 Hz, 1H), 2.51-2.72 (m, 3H), 1.80-1.89 (m, 2H), 1.51-1.69 (m, 2H), 1.35 (quin, J=6.6 Hz, 2H).

### Step G: 3⁴-Fluoro-2⁷-(trifluoromethyl)-1¹,1²,2¹,2²,2³,2⁴-hexahydro-2(3,1)-pyrido[4,3-d]pyrimidina-1(5,6)-pyridina-3(1,2)-benzenacyclooctaphane-1²,2⁴-dione Example 9

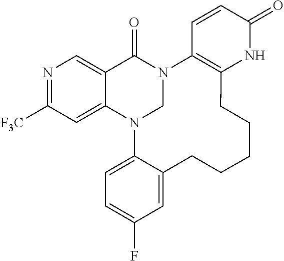 [SMILES: O=C1c2cnc(C(F)(F)F)cc2N2CN1c1ccc(=O)[nH]c1CCCCCc1cc(F)ccc12]

To a mixture of 3⁴-Fluoro-1⁶-methoxy-2⁷-(trifluoromethyl)-2¹,2²,2³,2⁴-tetrahydro-2(3,1)-pyrido[4,3-d]pyrimidina-1(3,2)-pyridina-3(1,2)-benzenacyclooctaphane-2⁴-one (73.0 mg, 0.150 mmol) and NaI (225 mg, 1.50 mmol) in MeCN (2.0 mL) was added TMS-C₁ (0.190 mL, 1.50 mmol) and the reaction mixture was heated at 60° C. for 2 h. The reaction mixture was cooled to ambient temperature, diluted with EtOAc (80 mL), washed with sat'd aq. NaHCO₃, aq. Na₂S₂O₃, brine, dried over MgSO₄, filtered and evaporated under reduced pressure. The residue was purified by silica gel flash column chromatography (40 g) eluting with a 100% heptanes to 80% 3:1 EtOAc-EtOH: heptane gradient. Product fractions were combined and evaporated under reduced pressure to afford 3⁴-fluoro-2⁷-(trifluoromethyl)-1¹,1²,2¹,2²,2³,2⁴-hexahydro-2(3,1)-pyrido[4,3-d]pyrimidina-1(5,6)-pyridina-3(1,2)-benzenacyclooctaphane-1²,2⁴-dione (46.6 mg, 62.4% yield) as a white solid. HPLC/MS 0.94 min (A), [M+H]⁺ 473.1. ¹H NMR (CD₃OD, 400 MHz) δ 9.03 (s, 1H), 7.56 (d, J=9.3 Hz, 1H), 7.39 (dd, J=5.4, 8.8 Hz, 1H), 7.24-7.14 (m, 2H), 6.56 (s, 1H), 6.44 (d, J=9.3 Hz, 1H), 5.82 (d, J=10.8 Hz, 1H), 4.94 (d, J=10.8 Hz, 1H), 2.68-2.47 (m, 4H), 1.98-1.81 (m, 2H), 1.64-1.48 (m, 2H), 1.40-1.33 (m, 2H).

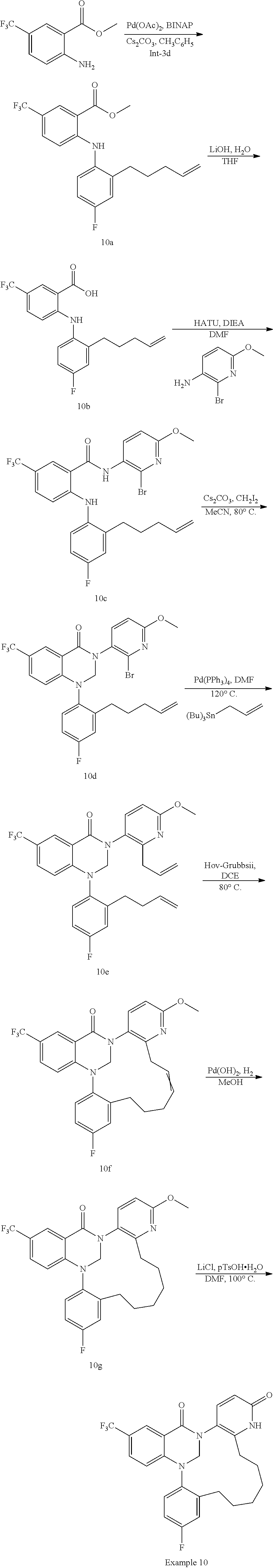

### Example 10

### 3⁴-Fluoro-2⁶-(trifluoromethyl)-1¹1²,2¹,2²,2³,2⁴-hexahydro-2(3,1)-quinazolina-1(5,6)-pyridina-3(1,2)-benzenacyclononaphane-1²,2⁴-dione

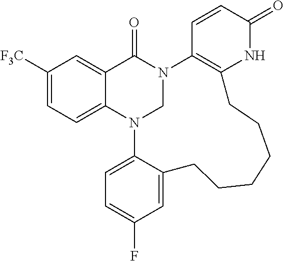 [SMILES: O=C1c2cc(C(F)(F)F)ccc2N2CN1c1ccc(=O)[nH]c1CCCCCCc1cc(F)ccc12]

### Step A: Methyl 2-((4-fluoro-2-(pent-4-en-1-yl)amino)-5-(trifluoromethyl)benzoate 10a

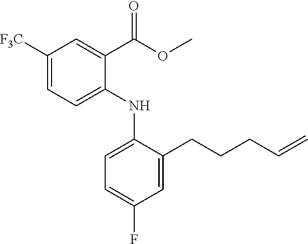 [SMILES: C=CCCCc1cc(F)ccc1Nc1ccc(C(F)(F)F)cc1C(=O)OC]

To a solution of 1-bromo-4-fluoro-2-(pent-4-en-1-yl)benzene Int-3d (9.00 g, 37.0 mmol) and methyl 2-amino-5-(trifluoromethyl)benzoate (9.74 g, 44.4 mmol) in toluene (90 mL), under N₂, were added Cs₂CO₃ (36.2 g, 111 mmol) and 2,2′-bis(diphenylphosphaneyl)-1,1′-binaphthalene (2.31 g, 3.70 mmol) at room temperature, the reaction mixture was purged with N₂ for 10 min, to which was added Pd₂(dba)₃ (1.70 g, 1.85 mmol) and the reaction mixture was stirred at 100° C. for 12 h. The reaction mixture was cooled to ambient temperature, filtered through celite, washed with EtOAc (2×50 mL), and the filtrate washed with H₂O (50 mL), brine, dried over Na₂SO₄, filtered, and the solvent evaporated under reduced pressure. The crude residue was dissolved in DCM, adsorbed onto silica gel and purified by silica gel flash column chromatography (100 g), eluting with 4% EtOAc-pet. ether. Product fractions were combined and evaporated under reduced pressure to afford methyl 2-((4-fluoro-2-(pent-4-en-1-yl)amino)-5-(trifluoromethyl)benzoate 10a (1.8 g, 11% yield) as a yellow oil. HPLC/MS 1.63 min (C), [M+H]⁺ 382.0.

### Step B: 2-((4-Fluoro-2-(pent-4-en-1-yl)phenyl)amino)-5-(trifluoromethyl)benzoic acid 10b

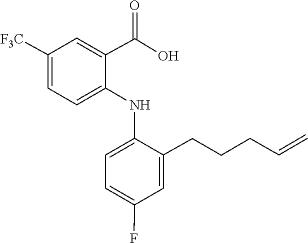 [SMILES: C=CCCCc1cc(F)ccc1Nc1ccc(C(F)(F)F)cc1C(=O)O]

Following the procedure outlined in Example 7, Step B, stirring the reaction mixture at room temperature for 12 h, 2-((4-fluoro-2-(pent-4-en-1-yl)phenyl)amino)-5-(trifluoromethyl)benzoic acid 10b (1.1 g, 56% yield) was prepared as a green solid. HPLC/MS 1.50 min (C), [M+H]⁺ 368.2. ¹H NMR (DMSO-d₆, 400 MHz) δ 13.6 (br s, 1H), 9.75 (s, 1H), 8.12 (s, 1H), 7.59-7.61 (d, 1H), 7.33-7.35 (m, 1H), 7.23-7.26 (m, 1H), 7.11-7.16 (m, 1H), 6.70-6.73 (m, 1H), 5.68-5.75 (m, 1H), 4.88-4.96 (m, 2H), 1.97-2.02 (m, 2H), 1.57-1.60 (m, 2H).

### Step C: N-(2-Bromo-6-methoxypyridin-3-yl)-2-((4-fluoro-2-(pent-4-en-1-yl)phenyl)-amino)-5-(trifluoro methyl)benzamide 10c

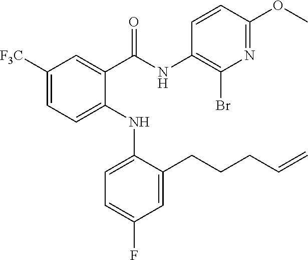 [SMILES: C=CCCCc1cc(F)ccc1Nc1ccc(C(F)(F)F)cc1C(=O)Nc1ccc(OC)nc1Br]

Following the procedure outlined in Example 3, Step C, stirring the reaction mixture at room temperature for 2 h, N-(2-bromo-6-methoxypyridin-3-yl)-2-((4-fluoro-2-(pent-4-en-1-yl)phenyl)amino)-5-(trifluoromethyl)benzamide 10c (1.0 g, 55% yield) was prepared as a brown gum. HPLC/MS 4.20 min (D), [M+H]⁺ 552.0. ¹H NMR (DMSO-d₆, 400 MHz) δ 10.4 (s, 1H), 9.74 (s, 1H), 8.27 (s, 1H), 7.83-7.85 (d, 1H), 7.59-7.62 (d, 1H), 7.31-7.35 (m, 1H), 7.20-7.23 (m, 2H), 7.10-7.14 (m, 1H), 6.95-6.98 (m, 1H), 6.77-6.79 (d, 1H), 5.67-5.76 (m, 1H), 4.87-4.94 (m, 2H), 3.89 (s, 3H), 1.95-2.00 (m, 2H), 1.54-1.58 (m, 2H).

### Step D: 3-(2-Bromo-6-methoxypyridin-3-yl)-1-(4-fluoro-2-(pent-4-en-1-yl)phenyl)-6-(trifluoromethyl)-2,3-dihydroquinazolin-4(11H)-one 10d

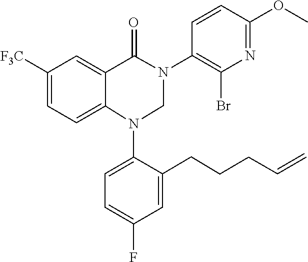 [SMILES: C=CCCCc1cc(F)ccc1N1CN(c2ccc(OC)nc2Br)C(=O)c2cc(C(F)(F)F)ccc21]

Following the procedure outlined in Example 1, Step C, stirring the reaction mixture at 80° C. for 10 h, 3-(2-bromo-6-methoxypyridin-3-yl)-1-(4-fluoro-2-(pent-4-en-1-yl)phenyl)-6-(trifluoromethyl)-2,3-dihydroquinazolin-4(1H)-one 10d (0.45 g, 33% yield) was prepared as a brown gum. HPLC/MS 1.50 min (C), [M+H]⁺ 563.8.

### Step E: 3-(2-Allyl-6-methoxypyridin-3-yl)-1-(4-fluoro-2-(pent-4-en-1-yl)phenyl)-6-(trifluoromethyl)-2,3-dihydroquinazolin-4(1H)-one 10e

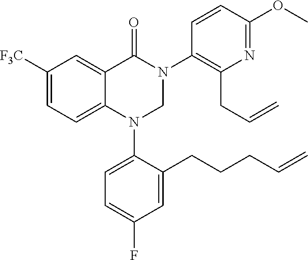 [SMILES: C=CCCCc1cc(F)ccc1N1CN(c2ccc(OC)nc2CC=C)C(=O)c2cc(C(F)(F)F)ccc21]

Following the procedure outlined in Example 3, Step E, stirring the reaction mixture at 150° C. for 1 h, 3-(2-allyl-6-methoxypyridin-3-yl)-1-(4-fluoro-2-(pent-4-en-1-yl)phenyl)-6-(trifluoromethyl)-2,3-dihydroquinazolin-4(1H)-one 10e (0.32 g) was prepared as a colorless liquid. HPLC/MS 1.53 min (C), [M+H]⁺ 525.8.

### Step F: 3⁴-Fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclononaphan-7-en-2⁴-one 10f

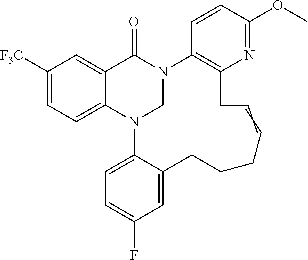 [SMILES: COc1ccc2c(n1)CC=CCCCc1cc(F)ccc1N1CN2C(=O)c2cc(C(F)(F)F)ccc21]

Following the procedure outlined in Example 7, Step E, stirring the reaction mixture at 80° C. for 12 h, 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclononaphan-7-en-2⁴-one 10f (0.200 g, 46% yield) was prepared as a green gum. HPLC/MS 1.48 min (C), [M+H]⁺ 498.4.

### Step G: 3⁴-Fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclononaphan-2⁴-one 102

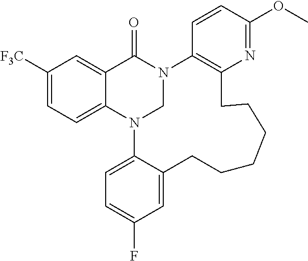 [SMILES: COc1ccc2c(n1)CCCCCCc1cc(F)ccc1N1CN2C(=O)c2cc(C(F)(F)F)ccc21]

Following the procedure outlined in Example 1, Step F, stirring the reaction mixture under H₂ (5 kg pressure) for 16 h, 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclononaphan-2⁴-one 10g (0.110 g, 71% yield) was prepared as a brown solid. HPLC/MS 1.50 min (C), [M+H]⁺ 499.8. ¹H NMR (DMSO-d₆, 400 MHz) δ 8.08 (s, 1H), 7.66-7.68 (d, 2H), 7.31-7.42 (m, 2H), 7.22-7.26 (m, 1H), 6.72-6.76 (m, 1H), 6.25-6.27 (m, 1H), 5.91-5.93 (d, 1H), 4.70-4.73 (d, 1H), 3.84 (s, 3H), 1.23-1.65 (m, 12H).

### 3⁴-Fluoro-2⁶-(trifluoromethyl)-1¹,1²,2¹,2²,2³,2⁴-hexahydro-2(3,1)-quinazolina-1(5,6)-pyridina-3(1,2)-benzenacyclononaphane-1²,2⁴-dione

### Example 10

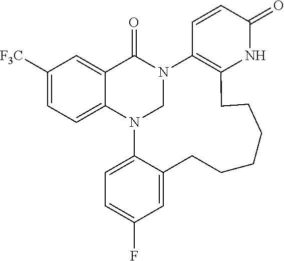 [SMILES: O=C1c2cc(C(F)(F)F)ccc2N2CN1c1ccc(=O)[nH]c1CCCCCCc1cc(F)ccc12]

Following the procedure outlined in Example 7, Step E, stirring the reaction mixture at 100° C. for 12 h, 3⁴-fluoro-1⁶-methoxy-2⁶-(trifluoromethyl)-2¹,2²,2³,2⁴-tetrahydro-2(3,1)-quinazolina-1(3,2)-pyridina-3(1,2)-benzenacyclononaphan-2⁴-one (4.9 mg, 6% yield) was prepared as an off-white solid. HPLC/MS 1.18 min (C), [M+H]⁺ 486.2. ¹H NMR (DMSO-d₆, 400 MHz) δ 11.82 (bs, 1H), 8.52 (s, 2H), 8.07 (d, J=1.60 Hz, 1H), 7.65 (dd, J=2.00, 8.80 Hz, 1H), 7.46-7.37 (m, 2H), 7.34-7.31 (m, 1H), 7.29-7.22 (m, 1H), 6.28-6.20 (m, 2H), 5.83 (d, J=9.20 Hz, 1H), 4.69 (d, J=9.20 Hz, 1H), 2.41-2.35 (m, 2H), 2.23-2.12 (m, 1H), 1.79-1.62 (m, 1H), 1.59-1.25 (m, 8H).

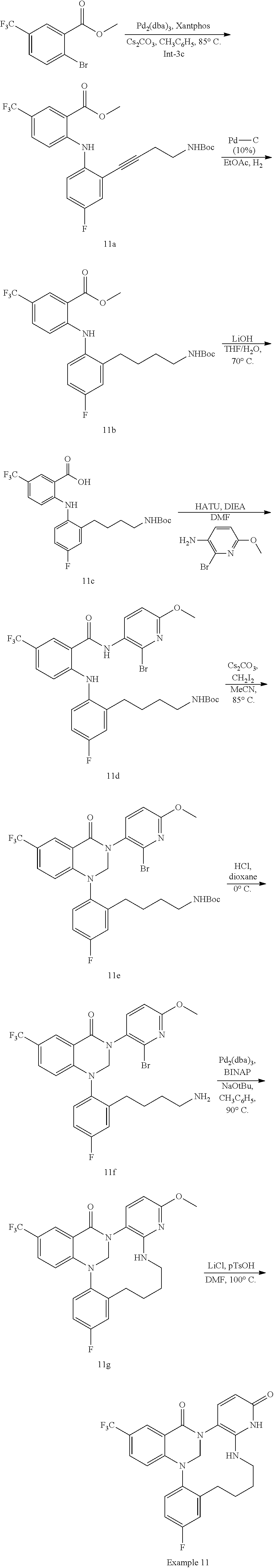

### Example 11

### 3⁴-Fluoro-2⁶-(trifluoromethyl)-1¹,1²,2¹,2²,2³,2⁴-hexahydro-8-aza-2(3,1)-quinazolina-1(5,6)-pyridina-3(1,2)-benzenacyclooctaphane-1²,2⁴-dione

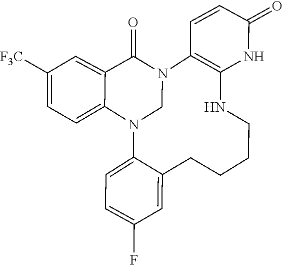 [SMILES: O=C1c2cc(C(F)(F)F)ccc2N2CN1c1ccc(=O)[nH]c1NCCCCc1cc(F)ccc12]

### Step A: Methyl 2-((2-(4-((tert-butoxycarbonyl)amino)but-1-yn-1-yl)-4-fluorophenll)-amino)-5-(trifluoromethyl)benzoate 11a

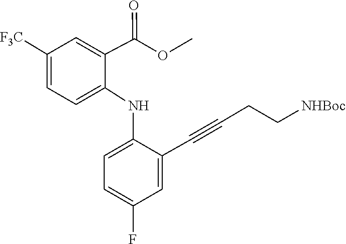 [SMILES: COC(=O)c1cc(C(F)(F)F)ccc1Nc1ccc(F)cc1C#CCCNC(=O)OC(C)(C)C]

To a stirred solution of tert-butyl (4-(2-amino-5-fluorophenyl)but-3-yn-1-yl)carbamate Int-3c (1.48 g, 5.32 mmol) and methyl 2-bromo-5-(trifluoromethyl)benzoate Int-1 (1.51 g, 5.32 mmol) in toluene (20 mL) were added Cs₂CO₃ (5.20 g, 15.9 mmol) and Xanthphos (0.308 g, 0.532 mmol) and the reaction mixture was purged with N₂ for 15 min, to which was added Pd₂(dba)₃ (0.243 g, 0.266 mmol) and the reaction mixture was stirred at 85° C. for 12 h. The reaction mixture was allowed to cool to ambient temperature, diluted with H₂O (30 mL) and extracted with EtOAc (2×30 mL). The combined organic extracts were washed with brine, dried over anhydrous Na₂SO₄, filtered and the solvent evaporated under reduced pressure. The crude product was dissolved in DCM, adsorbed on silica gel and purified by silica gel flash column chromatography (40 g), eluting with 7% of ethyl acetate-pet. ether. Product fractions were combined and evaporated under reduced pressure to afford methyl 2-((2-(4-((tert-butoxycarbonyl)amino)but-1-yn-1-yl)-4-fluorophenyl)amino)-5-(trifluoromethyl)benzoate 11a (2.2 g, 81% yield) as brown gum. HPLC/MS 1.47 min (C), [M+H]⁺ 479.2. ¹H NMR (DMSO-d₆, 400 MHz) δ 9.97 (s, 1H), 8.16-8.17 (s, 1H), 7.78-7.71 (m, 1H), 7.53-7.55 (m, 1H), 7.34-7.36 (m, 1H), 7.24-7.29 (m, 1H), 6.88-6.90 (m, 1H), 3.93 (s, 3H), 3.08-3.10 (m, 2H), 2.51 (m, 2H), 1.34 (s, 9H).

### Step B: Methyl 2-((2-(4-((tert-butoxycarbonyl)amino)butyl)-4-fluorophenyl)amino)-5-(trifluoromethyl) benzoate 11b

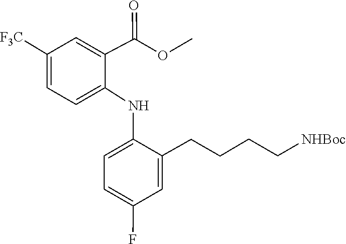 [SMILES: COC(=O)c1cc(C(F)(F)F)ccc1Nc1ccc(F)cc1CCCCNC(=O)OC(C)(C)C]

To stirred solution of methyl 2-((2-(4-((tert-butoxycarbonyl)amino)but-1-yn-1-yl)-4-fluorophenyl)amino)-5-(trifluoromethyl)benzoate (2.20 g, 4.58 mmol) in EtOAc (22 mL) was added 10% Pd—C(0.487 g, 0.458 mmol) at room temperature and the reaction mixture was stirred under H₂ atmosphere at 1 kg/cm² pressure for 2 h. The reaction mixture was filtered through celite, the celite washed with MeOH (2×10 mL), and the filtrate was concentrated under reduced pressure to afford methyl 2-((2-(4-((tert-butoxycarbonyl)amino)butyl)-4-fluorophenyl)amino)-5-(trifluoromethyl)benzoate 11b (2.1 g, 90% yield) as a brown gum. HPLC/MS 1.51 min (C), [M+H]⁺ 483.2. ¹H NMR (DMSO-d₆, 400 MHz) δ 9.42 (s, 1H), 8.12-8.13 (s, 1H), 7.61-7.64 (m, 1H), 7.31-7.34 (m, 1H), 7.23-7.26 (m, 1H), 7.11-7.16 (m, 1H), 6.67-6.72 (m, 2H), 3.91 (s, 3H), 2.51-2.54 (m, 2H), 2.09 (2, 2H), 1.46-1.50 (m, 2H), 1.34-1.44 (m, 11H).

### Step C: 2-((2-(4-((tert-Butoxycarbonyl)amino)butyl)-4-fluorophenyl)amino)-5-(trifluoromethyl)benzoic acid 11c

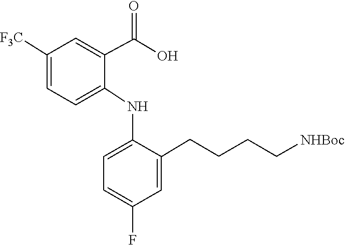 [SMILES: CC(C)(C)OC(=O)NCCCCc1cc(F)ccc1Nc1ccc(C(F)(F)F)cc1C(=O)O]

To a solution of methyl 2-((2-(4-((tert-butoxycarbonyl)amino)butyl)-4-fluorophenyl)amino)-5-(trifluoromethyl)benzoate (2.10 g, 4.33 mmol) in THF (20 mL) was added LiOH (0.728 g, 17.3 mmol) dissolved in water (6.67 mL), drop-wise, at 0° C. The resulting reaction mixture was heated at 70° C. for 6 h. The reaction mixture was cooled to ambient temperature, concentrated under reduced pressure, the resultant aqueous phase cooled to 0° C., acidified with sat'd aq. citric acid (7 mL), and extracted with EtOAc (2×50 mL). The combined organic extracts were washed with brine, dried over Na₂SO₄, filtered and the solvent evaporated under reduced pressure. The crude product was dissolved in DCM, adsorbed on silica gel and purified by silica gel flash column chromatography (40 g) eluting with 7% of ethyl acetate-pet. ether. Product fractions were combined and evaporated under reduced pressure to afford 2-((2-(4-((tert-butoxycarbonyl)amino)butyl)-4-fluorophenyl)amino)-5-(trifluoromethyl)benzoic acid 11c (1.8 g, 85% yield) as a brown gum. HPLC/MS 1.51 min (C), [M+H]⁺ 483.2. ¹H NMR (DMSO-d₆, 400 MHz) δ 13.5 (s, 1H), 9.75 (s, 1H), 8.12 (s, 1H), 7.59-7.61 (m, 1H), 7.32-7.36 (m, 1H), 7.22-7.25 (m, 1H), 7.10-7.15 (m, 1H), 6.71-6.74 (m, 2H), 2.74-2.87 (m, 2H), 2.52-2.54 (m, 2H), 1.46-1.49 (m, 2H), 1.34-1.37 (m, 11H).

### Step D: tert-Butyl (4-(2-((2-((2-bromo-6-methoxypyridin-3-yl)carbamoyl)-4-(trifluoro-methyl)phenyl)amino)-5-fluorophenyl)butyl)carbamate 11d

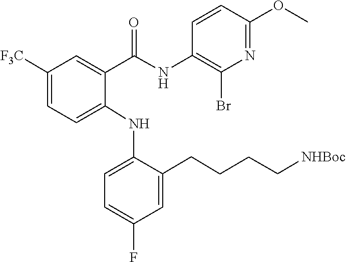 [SMILES: COc1ccc(NC(=O)c2cc(C(F)(F)F)ccc2Nc2ccc(F)cc2CCCCNC(=O)OC(C)(C)C)c(Br)n1]

To a solution of 2-((2-(4-((tert-butoxycarbonyl)amino)butyl)-4-fluorophenyl)amino)-5-(trifluoromethyl)benzoic acid (1.80 g, 3.83 mmol) and 2-bromo-6-methoxypyridin-3-amine (0.855 g, 4.21 mmol) in DMF (15 mL) was added DIEA (2.01 mL, 11.5 mmol) and HATU (2.91 g, 7.65 mmol) and the reaction mixture was stirred at room temperature for 3 h. The reaction mixture was diluted with ice cold water (60 mL), extracted with EtOAc (2×60 mL), and the combined organic extracts were washed with brine, dried over Na₂SO₄, filtered and the solvent evaporated under reduced pressure. The crude product was dissolved in DCM, adsorbed on silica gel and purified by silica gel flash column chromatography (40 g) eluting with 9% of ethyl acetate-pet. ether. Product fractions were combined and evaporated under reduced pressure to afford tert-butyl (4-(2-((2-((2-bromo-6-methoxypyridin-3-yl)carbamoyl)-4-(trifluoromethyl)phenyl)amino)-5-fluorophenyl)butyl)carbamate 11d (1.1 g, 42% yield) as a brown gum. HPLC/MS 1.48 min (C), [M+H]⁺ 655.0. ¹H NMR (DMSO-d₆, 400 MHz) δ 10.4 (s, 1H), 9.70 (s, 1H), 8.26 (s, 1H), 7.85-7.87 (d, 1H), 7.58-7.61 (d, 1H), 7.30-7.34 (m, 1H), 7.17-7.22 (m, 1H), 7.08-7.13 (m, 1H), 6.94-6.96 (d, 1H), 6.70-6.77 (d, 1H), 3.89 (s, 3H), 2.82-2.87 (m, 2H), 2.51 (m, 2H), 1.44-1.47 (m, 2H), 1.33 (m, 12H).

### Step E: tert-Butyl (4-(2-(3-(2-bromo-6-methoxypyridin-3-yl)-4-oxo-6-(trifluoromethyl)-3,4-dihydro quinazolin-1(2H)-yl)-5-fluorophenyl)butyl)carbamate 11e

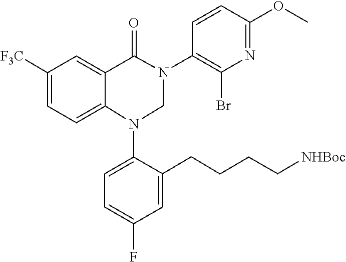 [SMILES: COc1ccc(N2CN(c3ccc(F)cc3CCCCNC(=O)OC(C)(C)C)c3ccc(C(F)(F)F)cc3C2=O)c(Br)n1]

To a solution of tert-butyl (4-(2-((2-((2-bromo-6-methoxypyridin-3-yl)carbamoyl)-4-(trifluoromethyl) phenyl)amino)-5-fluorophenyl)butyl)carbamate (1.00 g, 1.53 mmol) in MeCN (15 mL) was added Cs₂CO₃ (1.99 g, 6.10 mmol) and diiodomethane (0.369 mL, 4.58 mmol) and the reaction mixture was heated at 85° C. for 12 h. The reaction mixture was quenched with ice-cold water (20 mL), extracted with EtOAc (2×20 mL), and the combined extracts washed with water (40 mL), brine, dried over Na₂SO₄, filtered and the solvent evaporated under reduced pressure. The crude product was dissolved in DCM, adsorbed on silica gel and purified by silica gel flash column chromatography (40 g) eluting with 14% of ethyl acetate-pet. ether. Product fractions were combined and evaporated under reduced pressure to afford tert-butyl (4-(2-(3-(2-bromo-6-methoxypyridin-3-yl)-4-oxo-6-(trifluoromethyl)-3,4-dihydroquinazolin-1(2H)-yl)-5-fluorophenyl)butyl)carbamate 11e (0.52 g, 40% yield) as a brown gum. HPLC/MS 1.44 min (C), [M+H]⁺ 669.0. ¹H NMR (DMSO-d₆, 400 MHz) δ 8.08 (s, 1H), 7.81-7.89 (m, 1H), 7.64-7.67 (m, 1H), 7.32-7.43 (m, 2H), 7.20-7.22 (m, 1H), 6.98-7.00 (m, 1H), 6.72-6.75 (m, 1H), 6.30-6.40 (dd, 1H), 5.31-5.65 (dd, 1H), 4.75-4.77 (dd, 1H), 3.88 (s, 3H), 2.84-2.86 (m, 2H), 2.65-2.86 (m, 2H), 1.52-1.54 (m, 2H), 1.32-1.34 (m, 11H).

### Step F: 1-(2-(4-Aminobutyl)-4-fluorophenyl)-3-(2-bromo-6-methoxypyridin-3-yl)-6-(trifluoromethyl)-2,3-dihydroquinazolin-4(11H)-one, hydrochloride salt 11f

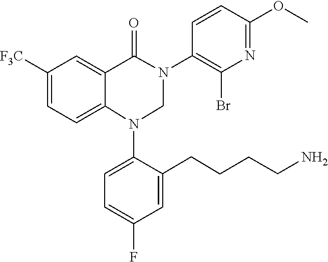 [SMILES: COc1ccc(N2CN(c3ccc(F)cc3CCCCN)c3ccc(C(F)(F)F)cc3C2=O)c(Br)n1]

To a solution of tert-butyl (4-(2-(3-(2-bromo-6-methoxypyridin-3-yl)-4-oxo-6-(trifluoromethyl)-3,4-dihydroquinazolin-1(2H)-yl)-5-fluorophenyl)butyl)carbamate (0.520 g, 0.615 mmol) in 1,4-dioxane (5 mL), cooled to 0° C., was added 4 M HCl/dioxane sol'n (1.54 mL, 6.15 mmol) and the reaction mixture was stirred at room temperature for 1 h. The reaction mixture was concentrated under reduced pressure to afford 1-(2-(4-aminobutyl)-4-fluorophenyl)-3-(2-bromo-6-methoxypyridin-3-yl)-6-(trifluoromethyl)-2,3-dihydroquinazolin-4(1H)-one, hydrochloride 11f (0.35 g, 85% yield) as a brown solid. HPLC/MS 1.05 min (C), [M+H]f 669.0. ¹H NMR (DMSO-d₆, 400 MHz) δ 8.09 (s, 1H), 7.66-7.91 (m, 6H), 7.23-7.44 (m, 3H), 6.99-7.02 (d, 1H), 6.31-6.33 (dd, 1H), 5.67-6.33 (dd, 1H), 4.73-6.33 (dd, 1H), 3.88 (s, 3H), 2.58-2.70 (m, 2H), 1.52-1.62 (m, 4H).

### Step G: 3⁴-Fluoro-1⁶-methoxy-2⁶-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 11

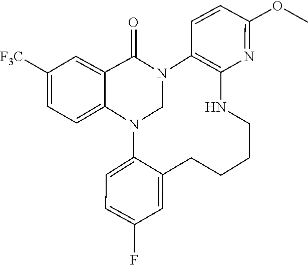 [SMILES: COc1ccc2c(n1)NCCCCc1cc(F)ccc1N1CN2C(=O)c2cc(C(F)(F)F)ccc21]

A solution of 1-(2-(4-aminobutyl)-4-fluorophenyl)-3-(2-bromo-6-methoxypyridin-3-yl)-6-(trifluoromethyl)-2,3-dihydroquinazolin-4(1H)-one, hydrochloride (300 mg, 0.497 mmol) and sodium tert-butoxide (239 mg, 2.48 mmol) in toluene (7 mL) was purged with N₂ for 20 min, at which time BINAP (30.9 mg, 0.050 mmol) and Pd₂(dba)₃ (22.8 mg, 0.025 mmol) were added, the reaction mixture purged for 5 min, then heated at 90° C. for 3 h. The reaction mixture was cooled to ambient temperature, quenched with ice-cold water (20 mL) and extracted with EtOAc (2×30 mL). The combined extracts were with water (40 mL), brine, dried over Na₂SO₄, filtered and the solvent evaporated under reduced pressure. The crude product was dissolved in DCM, adsorbed on silica gel and purified by silica gel flash column chromatography (40 g) eluting with 17% of ethyl acetate-pet. ether. Product fractions were combined and evaporated under reduced pressure to afford 3⁴-fluoro-1⁶-methoxy-2⁶-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 11g (0.162 g, 40% yield) as a white solid. HPLC/MS 1.40 min (C), [M+H]⁺ 487.0. ¹H NMR (DMSO-d₆, 400 MHz) δ 8.11 (s, 1H), 7.62-7.64 (d, 1H), 7.35-7.45 (m, 3H), 7.18-7.23 (t, 1H), 6.28-6.31 (d, 1H), 6.00-6.02 (d, 1H), 5.91-5.94 (m, 1H), 5.45-5.47 (d, 1H), 4.40-4.42 (d, 1H), 3.77 (s, 3H), 3.49-3.51 (m, 1H), 3.29-3.33 (m, 1H), 2.67 (m, 1H), 1.62-1.85 (m, 2H), 1.40-1.60 (m, 2H).

### Step H: 3⁴-Fluoro-2⁶-(trifluoromethyl)-1¹,1²,2¹,2²,2³,2⁴-hexahydro-8-aza-2(3,1)-quinazolina-1(5,6)-pyridina-3(1,2)-benzenacyclooctaphane-1²,2⁴-dione Example 11

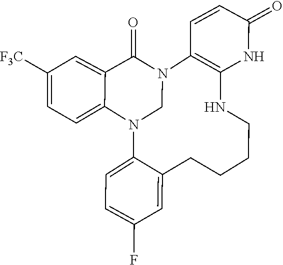 [SMILES: O=C1c2cc(C(F)(F)F)ccc2N2CN1c1ccc(=O)[nH]c1NCCCCc1cc(F)ccc12]

To a solution of 3⁴-fluoro-1⁶-methoxy-2⁶-(trifluoromethyl)-2¹,2²,2³,2⁴-tetrahydro-8-aza-2(3,1)-quinazolina-1(3,2)-pyridina-3(1,2)-benzenacyclooctaphan-2⁴-one (162 mg, 0.333 mmol) in DMF (5 mL) was added LiCl (85 mg, 1.99 mmol) and p-TsOH (380 mg, 1.99 mmol) and the reaction mixture was stirred at 100° C. for 4 h. The reaction mixture was cooled to ambient temperature, quenched with ice-cold water (40 mL), extracted with EtOAc (2×20 mL), and the combined extracts were washed with brine, dried over Na₂SO₄, filtered and the solvent evaporated under reduced pressure. The crude product was dissolved in DCM, adsorbed on silica gel and purified by silica gel flash column chromatography (40 g) eluting with 17% ethyl acetate-pet. ether. The isolated material was purified by semi-prep reverse-phase HPLC (YMC Actus Triart C_{18, 250×30} mm, 5 m) eluting with a 50% MeCN—H₂O (0.1% formic acid) to 100% MeCN (0.10% formic acid) gradient. Product peaks were combined, concentrated under reduced pressure and dried under high vacuum to afford 3⁴-fluoro-2⁶-(trifluoromethyl)-1¹,1²,2¹,2²2³,2⁴-hexahydro-8-aza-2(3,1)-quinazolina-1(5,6)-pyridina-3(1,2)-benzenacyclooctaphane-1²,2⁴-dione (42.3 mg, 27% yield) as a light yellow solid. HPLC/MS 1.08 min (C), [M+H]⁺ 487.0. ¹H NMR (DMSO-d₆, 400 MHz) δ 10.44 (bs, 1H), 8.10 (s, 1H), 7.63 (dd, J=1.60, 8.60 Hz, 1H), 7.44-7.35 (m, 2H), 7.29-7.19 (m, 2H), 6.28 (d, J=8.80 Hz, 1H), 5.82-5.69 (m, 2H), 5.50 (d, J=10.40 Hz, 1H), 4.45 (d, J=10.00 Hz, 1H), 3.44 (s, 1H), 3.26 (s, 1H), 2.88 (s, 1H), 2.45 (s, 1H), 1.77-1.48 (m, 4H).

### Example 12

### 3⁴-Fluoro-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphane-1²,2⁴-dione

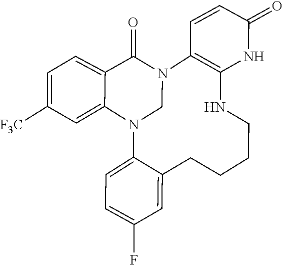 [SMILES: O=C1c2ccc(C(F)(F)F)cc2N2CN1c1ccc(=O)[nH]c1NCCCCc1cc(F)ccc12]

### Step A: Methyl 2-((2-(4-((tert-butoxycarbonyl)amino)but-1-yn-1-yl)-4-fluorophenyl)-amino)-4-(trifluoromethyl)benzoate 12a

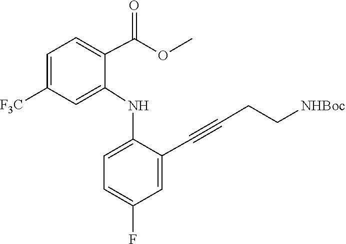 [SMILES: COC(=O)c1ccc(C(F)(F)F)cc1Nc1ccc(F)cc1C#CCCNC(=O)OC(C)(C)C]

Following the procedure outlined in Example 11, Step A, substituting Int-2 with Int-2c, Pd₂(dba)₃ with Pd(OAc)₂ and stirring the reaction mixture at 95° C. for 7 h, methyl 2-((2-(4-((tert-butoxycarbonyl)amino)but-1-yn-1-yl)-4-fluorophenyl)amino)-4-(trifluoromethyl)benzoate 12a (3.1 g, 59% yield), was prepared as a yellow oil. HPLC/MS 1.54 min (C), [M+H]⁺ 481.1. ¹H NMR (DMSO-d₆, 400 MHz) δ 9.75 (s, 1H), 8.11 (d, 1H, J=7.8 Hz), 7.50 (dd, 1H, J=5.1, 9.0 Hz), 7.2-7.4 (m, 3H), 7.15 (dd, 1H, J=1.5, 8.3 Hz), 6.91 (br t, 1H, J=5.9 Hz), 3.92 (s, 3H), 3.08 (q, 2H, J=6.8 Hz), 2.5-2.5 (m, 2H), 1.33 (s, 9H).

### Step B: Methyl 2-((2-(4-((tert-butoxycarbonyl)amino)butyl)-4-fluorophenyl)amino)-4-(trifluoromethyl)benzoate 12b

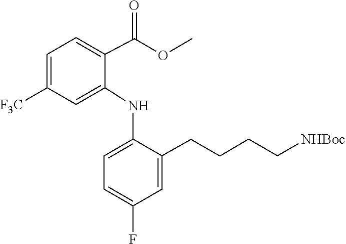 [SMILES: COC(=O)c1ccc(C(F)(F)F)cc1Nc1ccc(F)cc1CCCCNC(=O)OC(C)(C)C]

Following the procedure outlined in Example 11, Step B, stirring the reaction mixture for 1 h, methyl 2-((2-(4-((tert-butoxycarbonyl)amino)butyl)-4-fluorophenyl)amino)-4-(trifluoromethyl)benzoate 12b (3.37 g, 100% yield), was prepared as a yellow oil. HPLC/MS 1.54 min (C), [M+H]⁺ 485.0. ¹H NMR (CD₃CN, 400 MHz) δ 9.29 (s, 1H), 8.11 (d, 1H, J=8.3 Hz), 7.31 (dd, 1H, J=5.4, 8.3 Hz), 7.16 (dd, 1H, J=2.9, 9.8 Hz), 7.05 (dt, 1H, J=2.9, 8.6 Hz), 6.97 (dd, 1H, J=1.5, 8.3 Hz), 6.86 (d, 1H, J=1.0 Hz), 5.21 (br s, 1H), 3.95 (s, 3H), 2.99 (q, 2H, J=6.7 Hz), 2.6-2.6 (m, 2H), 1.5-1.6 (m, 2H), 1.4-1.5 (m, 11H).

### Step C: 2-((2-(4-((tert-Butoxycarbonyl)amino)butyl)-4-fluorophenl)amino)-4-(trifluoromethyl)benzoic acid 12c

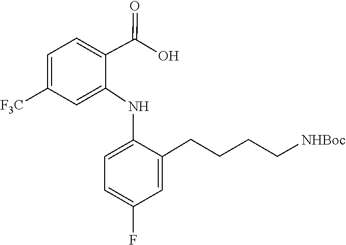 [SMILES: CC(C)(C)OC(=O)NCCCCc1cc(F)ccc1Nc1cc(C(F)(F)F)ccc1C(=O)O]

Following the procedure outlined in Example 11, Step C, substituting LiOH with 1M NaOH and stirring the reaction mixture at room temperature for 72 h, methyl 2-((2-(4-((tert-butoxycarbonyl) amino)butyl)-4-fluorophenyl)amino)-4-(trifluoromethyl)benzoate 12c (3.57 g, 89% yield), in 75% purity, was prepared as a yellow oil. HPLC/MS 1.45 min (C), [M+H]⁺ 469.2. ¹H NMR (DMSO-d₆, 400 MHz) δ 13.60 (br s, 1H), 9.57 (s, 1H), 8.07 (d, 1H, J=8.3 Hz), 7.3-7.4 (m, 1H), 7.2-7.3 (m, 1H), 7.1-7.2 (m, 1H), 6.9-7.0 (m, 1H), 6.78 (s, 1H), 6.73 (br t, 1H, J=4.9 Hz), 2.5-2.6 (m, 2H), 1.4-1.6 (m, 2H), 1.34 (s, 11H).

### Step D: tert-Butyl (4-(2-((2-((2-bromo-6-methoxypyridin-3-yl)carbamoyl)-5-(trifluoro-methyl)phenyl) amino)-5-fluorophenyl)butyl)carbamate 12d

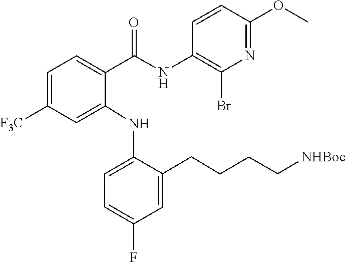 [SMILES: COc1ccc(NC(=O)c2ccc(C(F)(F)F)cc2Nc2ccc(F)cc2CCCCNC(=O)OC(C)(C)C)c(Br)n1]

Following the procedure outlined in Example 11, Step D, stirring at room temperature for 6 h, tert-butyl (4-(2-((2-((2-bromo-6-methoxypyridin-3-yl)carbamoyl)-5-(trifluoromethyl)phenyl)amino)-5-fluorophenyl)butyl)carbamate 12d (563 mg, 53% yield), was prepared as a red solid. HPLC/MS 1.59 min (C), [M+H]⁺ 655.0. ¹H NMR (DMSO-d₆, 400 MHz) δ 10.39 (s, 1H), 9.33 (s, 1H), 8.07 (d, 1H, J=8.3 Hz), 7.85 (d, 1H, J=8.3 Hz), 7.32 (dd, 1H, J=5.6, 8.6 Hz), 7.20 (dd, 1H, J=2.9, 9.8 Hz), 7.0-7.2 (m, 2H), 6.95 (d, 1H, J=8.3 Hz), 6.86 (d, 1H, J=1.0 Hz), 6.70 (br t, 1H, J=5.6 Hz), 3.88 (s, 3H), 3.3-3.3 (m, 2H), 2.84 (q, 2H, J=6.7 Hz), 1.4-1.5 (m, 2H), 1.33 (s, 11H).

### Step E: tert-Butyl (4-(2-(3-(2-bromo-6-methoxypyridin-3-yl)-4-oxo-7-(trifluoromethyl)-3,4-dihydroquinazolin-1(2H)-yl)-5-fluorophenyl)butyl)carbamate 12e

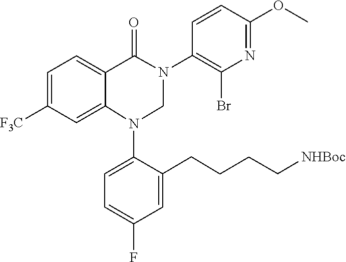 [SMILES: COc1ccc(N2CN(c3ccc(F)cc3CCCCNC(=O)OC(C)(C)C)c3cc(C(F)(F)F)ccc3C2=O)c(Br)n1]

Following the procedure outlined in Example 11, Step E, stirring the reaction mixture at 65° C. for 22 h then 85° C. for 7 h, tert-butyl (4-(2-(3-(2-bromo-6-methoxypyridin-3-yl)-4-oxo-7-(trifluoromethyl)-3,4-dihydroquinazolin-1(2H)-yl)-5-fluorophenyl)butyl)carbamate 12e (169 mg, 32% yield), was prepared as an off-white solid. HPLC/MS 1.47 min (C), [M+H]⁺ 666.0. ¹H NMR (CD₃CN, 400 MHz) δ 8.13 (d, 1H, J=8.3 Hz), 7.71 (br d, 1H, J=7.3 Hz), 7.3-7.4 (m, 1H), 7.20 (br d, 2H, J=8.3 Hz), 7.10 (br t, 1H, J=7.6 Hz), 6.8-6.9 (m, 1H), 6.4-6.6 (m, 1H), 5.3-5.5 (m, 1H), 5.20 (br s, 1H), 4.8-5.1 (m, 1H), 3.93 (s, 3H), 2.97 (q, 2H, J=6.4 Hz), 2.6-2.8 (m, 2H), 1.5-1.7 (m, 2H), 1.43 (br s, 2H), 1.38 (s, 9H).

### Step F: 1-(2-(4-Aminobutyl)-4-fluorophenyl)-3-(2-bromo-6-methoxypyridin-3-yl)-7-(trifluoromethyl)-2,3-dihydroquinazolin-4(11H)-one 12f

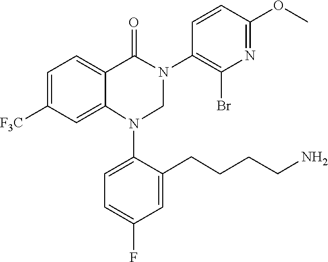 [SMILES: COc1ccc(N2CN(c3ccc(F)cc3CCCCN)c3cc(C(F)(F)F)ccc3C2=O)c(Br)n1]

Following the procedure outlined in Example 11, Step F, stirring the reaction mixture in 4N HCl/dioxane for 1 h, to which was added 1.25N HCl/MeOH and the reaction mixture stirred for an additional 72 h, 1-(2-(4-aminobutyl)-4-fluorophenyl)-3-(2-bromo-6-methoxypyridin-3-yl)-7-(trifluoromethyl)-2,3-dihydroquinazolin-4(1H)-one 12f (169 mg, 32% yield), was prepared as an orange semi-solid. HPLC/MS 0.80 min (C), [M+H]⁺ 567.0. ¹H NMR (DMSO-d₆, 400 MHz) δ 8.1-8.1 (m, 1H), 7.8-7.9 (m, 1H), 7.3-7.5 (m, 2H), 7.2-7.3 (m, 2H), 6.9-7.0 (m, 1H), 6.3-6.5 (m, 1H), 5.3-5.7 (m, 1H), 4.7-5.2 (m, 1H), 3.88 (s, 3H), 3.4-3.4 (m, 2H), 2.6-2.8 (m, 4H), 1.4-1.7 (m, 4H).

### Step G: 3⁴-Fluoro-1⁶-methoxy-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 122

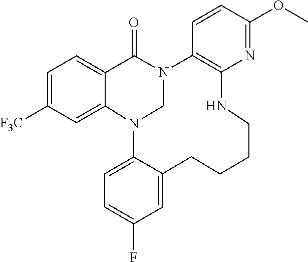 [SMILES: COc1ccc2c(n1)NCCCCc1cc(F)ccc1N1CN2C(=O)c2ccc(C(F)(F)F)cc21]

Following the procedure outlined in Example 11, Step G, stirring the reaction mixture at 90° C. for 2 h, 3⁴-fluoro-1⁶-methoxy-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 12g (60 mg, 46% yield), was prepared as a yellow solid. HPLC/MS 1.42 min (C), [M+H]⁺ 487.0. ¹H NMR (CD₃CN, 400 MHz) δ 8.12 (d, 1H, J=8.3 Hz), 7.40 (d, 1H, J=8.3 Hz), 7.29 (dd, 1H, J=5.4, 8.8 Hz), 7.25 (dd, 1H, J=2.9, 9.8 Hz), 7.19 (dd, 1H, J=1.0, 7.8 Hz), 7.10 (dt, 1H, J=3.2, 8.4 Hz), 6.45 (s, 1H), 6.00 (d, 1H, J=8.3 Hz), 5.35 (d, 2H, J=10.8 Hz), 4.40 (d, 1H, J=10.8 Hz), 3.82 (s, 3H), 3.6-3.8 (m, 1H), 3.2-3.3 (m, 1H), 2.9-3.0 (m, 1H), 2.51 (ddd, 1H, J=5.9, 10.5, 13.5 Hz), 1.7-1.9 (m, 2H), 1.5-1.6 (m, 2H).

### Step H: 3⁴-Fluoro-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphan-1²,2⁴-dione

### Example 12

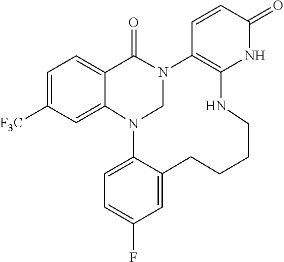 [SMILES: O=C1c2ccc(C(F)(F)F)cc2N2CN1c1ccc(=O)[nH]c1NCCCCc1cc(F)ccc12]

Following the procedure outlined in Example 11, Step H, stirring the reaction mixture at 90° C. for 15.5 h, then 100° C. for 40 min, 3⁴-fluoro-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphan-1²,2⁴-dione (34 mg, 55% yield), was prepared as a light yellow solid. HPLC/MS 1.07 min (C), [M+H]⁺ 473.2. ¹H NMR (CD₃CN, 400 MHz) δ 8.15 (d, 1H, J=7.8 Hz), 7.67 (d, 1H, J=8.8 Hz), 7.34 (dd, 1H, J=5.4, 8.8 Hz), 7.27 (dd, 1H, J=2.9, 9.8 Hz), 7.22 (d, 1H, J=7.8 Hz), 7.14 (dt, 1H, J=2.9, 8.6 Hz), 6.48 (s, 1H), 6.05 (d, 1H, J=8.8 Hz), 5.40 (d, 1H, J=10.8 Hz), 4.46 (d, 1H, J=10.8 Hz), 3.57 (td, 1H, J=6.2, 13.0 Hz), 3.44 (td, 1H, J=6.6, 13.2 Hz), 2.9-3.0 (m, 1H), 2.5-2.6 (m, 1H), 2.0-2.0 (m, 2H), 1.8-1.9 (m, 2H), 1.4-1.7 (m, 2H).

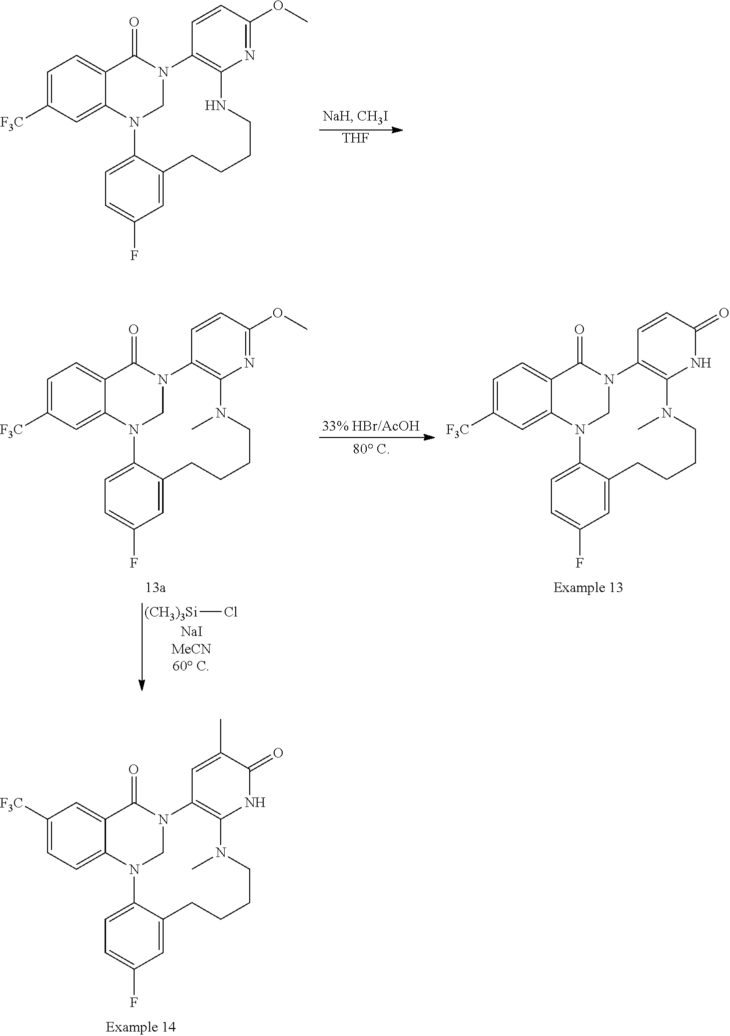

### Example 13

### 3⁴-Fluoro-8-methyl-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphan-1²,2⁴-dione

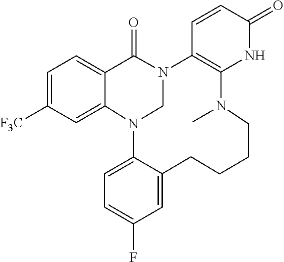 [SMILES: CN1CCCCc2cc(F)ccc2N2CN(C(=O)c3ccc(C(F)(F)F)cc32)c2ccc(=O)[nH]c21]

### Step A: 3⁴-Fluoro-1⁶-methoxy-8-methyl-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 13a

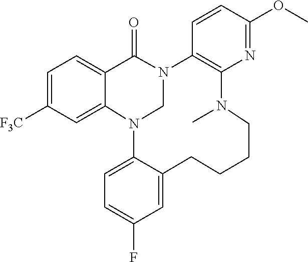 [SMILES: COc1ccc2c(n1)N(C)CCCCc1cc(F)ccc1N1CN2C(=O)c2ccc(C(F)(F)F)cc21]

A solution of 3⁴-fluoro-1⁶-methoxy-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one (56.0 mg, 0.115 mmol) in THF (4 mL) was treated with 60% NaH (92 mg, 2.30 mmol), the reaction mixture stirred at room temperature for 1 min, to which was added CH₃I (0.036 mL, 0.576 mmol) and the reaction mixture was stirred at room temperature for 4 h. The reaction mixture was diluted with EtOAc, washed with H₂O, brine, the organic layer dried over Na₂SO₄, filtered and the solvent evaporated under reduced pressure. The residue was dissolved in DCM and purified by silica gel flash column chromatography (24 g), eluting with a 100% heptanes to 25% EtOAc-heptanes gradient. The product fractions were combined, evaporated under reduced pressure and dried under vacuo to afford 3⁴-fluoro-1⁶-methoxy-8-methyl-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one 13a (48 mg, 73% yield) as a white solid. HPLC/MS 1.45 min (A), [M+H]⁺ 501.2. ¹H NMR (CD₃CN, 400 MHz) δ 8.14 (d, 1H, J=8.3 Hz), 7.65 (dd, 1H, J=5.4, 8.8 Hz), 7.4-7.4 (m, 1H), 7.31 (dd, 1H, J=5.4, 8.8 Hz), 7.2-7.2 (m, 3H), 7.0-7.0 (m, 1H), 6.45 (s, 1H), 6.13 (d, 1H, J=8.3 Hz), 5.62 (d, 1H, J=9.8 Hz), 4.74 (d, 1H, J=10.3 Hz), 3.88 (s, 3H), 3.04 (s, 3H), 2.0-2.1 (m, 2H), 1.6-1.8 (m, 4H).

### Step B: 3⁴-Fluoro-8-methyl-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphan-1²,2⁴-dione Example 13

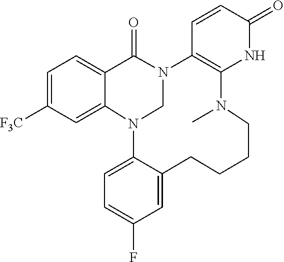 [SMILES: CN1CCCCc2cc(F)ccc2N2CN(C(=O)c3ccc(C(F)(F)F)cc32)c2ccc(=O)[nH]c21]

A suspension of 3⁴-fluoro-1⁶-methoxy-8-methyl-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one (48.0 mg, 0.096 mmol) in AcOH (10 mL) was treated with 33% HBr in AcOH (0.158 mL, 0.959 mmol) and the reaction mixture heated at 80° C. for 12 h, then stirred at room temperature for 4 h. The solvent was evaporated under reduced pressure and the residue azeotroped with toluene several times, then dissolved in DCM and MeOH and purified by silica gel flash column chromatography (24 g) eluting with a 100% heptanes to 20% EtOAc-heptanes gradient, followed by a 100% DCM to 100% 3:1 EtOAc/EtOH gradient. The product fractions were combined and evaporated under reduced pressure to afford 3⁴-fluoro-8-methyl-2⁷-(trifluoromethyl)-1¹,1²,2¹,2²,2³,2⁴-hexahydro-8-aza-2(3,1)-quinazolina-1(5,6)-pyridina-3(1,2)-benzenacyclooctaphan-1²,2⁴-dione (18 mg, 37% yield) as a white solid. HPLC/MS 1.10 min (A), [M+H]⁺ 487.0. ¹H-NMR (400 MHz, CD₂Cl₂) δ 8.14 (br d, 1H, J=7.8 Hz), 7.29 (br s, 1H), 7.0-7.2 (m, 4H), 6.46 (s, 1H), 5.95 (br s, 1H), 5.45 (br d, 1H, J=2.9 Hz), 5.32 (s, 1H), 4.58 (br d, 1H, J=6.8 Hz), 4.24 (br s, 1H), 2.98 (br s, 3H), 2.82 (br d, 1H, J=8.8 Hz), 2.6-2.8 (m, 1H), 2.4-2.5 (m, 1H), 1.76 (br s, 1H), 1.62 (br s, 2H), 1.3-1.5 (m, 1H).

### Example 14

### 3⁴-Fluoro-1³-iodo-8-methyl-2⁷-(trifluoromethyl)-1¹, 1²,2¹,2²,2³,2⁴-hexahydro-8-aza-2(3,1)-quinazolina-1(5,6)-pyridina-3(1,2)-benzenacyclooctaphan-1²,2⁴-dione

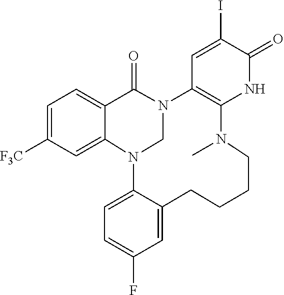 [SMILES: CN1CCCCc2cc(F)ccc2N2CN(C(=O)c3ccc(C(F)(F)F)cc32)c2cc(I)c(=O)[nH]c21]

A solution of 3⁴-fluoro-1⁶-methoxy-8-methyl-2⁷-(trifluoromethyl)-2¹,2²,2³,2⁴-tetrahydro-8-aza-2(3,1)-quinazolina-1(3,2)-pyridina-3(1,2)-benzenacyclooctaphan-2⁴-one (23.0 mg, 0.046 mmol) in MeCN (2 mL) was treated with chlorotrimethylsilane (5.83 μl, 0.046 mmol) and NaI (6.89 mg, 0.046 mmol) and the reaction mixture was heated at 60° C. for 24 h. Additional MeCN (2 mL), NaI (6.89 mg, 0.046 mmol), and chlorotrimethylsilane (5.83 μl, 0.046 mmol) were added and the reaction heated at 60° C. for an additional 24 h, to which was added additional NaI (6.89 mg, 0.046 mmol) and chlorotrimethylsilane (5.83 μl, 0.046 mmol), the reaction mixture heated at 60° C. for 24 h, followed by further addition of NaI (6.89 mg, 0.046 mmol) and chlorotrimethylsilane (5.83 μl, 0.046 mmol) and continued heating at 60° C. for 4 h. The reaction mixture was cooled to ambient temperature and the solvent evaporated under reduced pressure. The crude residue was dissolved in DMSO/MeOH (3:1 mL) and purified by semi-prep reverse phase (XSELECT, CSH C₁₈ column (150 mm×30 mm, 5 μm) eluting with a 30:70 MeCN—H₂O (0.1% TFA) to 99:1 MeCN—H₂O (0.1% TFA) gradient. The product fractions were combined, the solvent evaporated under reduced pressure and dried under vacuo to afford 3⁴-fluoro-1³-iodo-8-methyl-2⁷-(trifluoromethyl)-1¹, 1²,2¹,2²,2³,2⁴-hexahydro-8-aza-2(3,1)-quinazolina-1(5,6)-pyridina-3(1,2)-benzenacyclooctaphan-1²,2⁴-dione (10 mg, 32% yield) as a yellowish-brown solid. HPLC/MS 1.24 min (A), [M+H]⁺ 613.1. ¹H-NMR (CD₂Cl₂, 400 MHz) δ 8.16 (d, 1H, J=8.3 Hz), 7.81 (s, 1H), 7.2-7.2 (m, 2H), 7.1-7.1 (m, 2H), 6.46 (s, 1H), 5.54 (d, 1H, J=9.8 Hz), 4.60 (d, 1H, J=9.8 Hz), 4.5-4.6 (m, 1H), 3.07 (s, 3H), 2.8-2.9 (m, 1H), 2.5-2.6 (m, 1H), 2.4-2.5 (m, 1H), 1.6-1.8 (m, 4H), 1.36 (dt, 1H, J=4.6, 10.6 Hz).

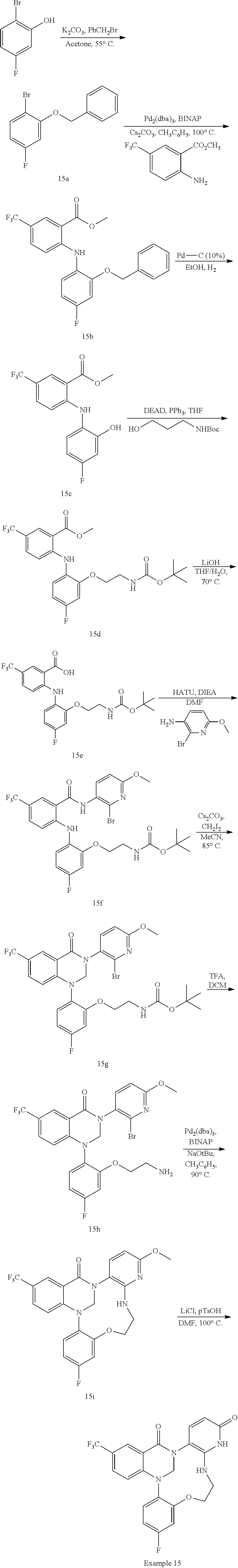

### Example 15

### 8-Fluoro-2-(trifluoromethyl)-12,13-dihydro-11H,19H-5,18-methanodibenzo[i,j]pyrido[2,3-e][1]oxa[4,7,11]triazacyclotridecine-15,19(14H)-dione

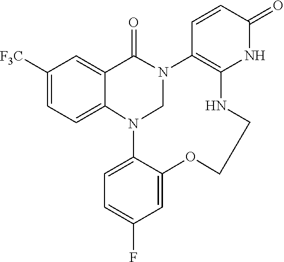 [SMILES: O=C1c2cc(C(F)(F)F)ccc2N2CN1c1ccc(=O)[nH]c1NCCOc1cc(F)ccc12]

### Step A: 2-(Benzyloxy)-1-bromo-4-fluorobenzene 15a

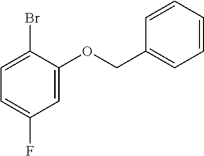 [SMILES: Fc1ccc(Br)c(OCc2ccccc2)c1]

To a solution of 2-bromo-5-fluorophenol (10.0 g, 52.4 mmol) in acetone (70 mL), under N₂, was added K₂CO₃ (7.60 g, 55.0 mmol) portion-wise, followed by (bromomethyl)benzene (6.22 mL, 52.4 mmol), and the resultant suspension was stirred at 55° C. for 3 h. The reaction mixture was allowed to cool to ambient temperature and concentrated under vacuo to obtain yellow gum residue which was quenched with ice water (100 mL), acidified with 1.5 N HCl (60 mL) to pH 3-4, and extracted with ethyl acetate (2×100 mL). The combined organic extracts were washed with brine, dried over Na₂SO₄, filtered and evaporated under reduced pressure. The crude residue was preabsorbed onto silica gel and purified by silica gel flash column chromatography (350 g), eluting with a 0-2% EtOAc in pet-ether gradient. Product fractions were combined and evaporated under reduced pressure to afford 2-(benzyloxy)-1-bromo-4-fluorobenzene 15a (13.7 g, 92% yield) as a yellow oil. HPLC/MS 1.30 min (C), [M+H]⁺ 278.8. ¹H NMR (DMSO-d₆, 400 MHz) δ 7.60-7.62 (dd, 1H), 7.47-7.49 (m, 2H), 7.43-7.44 (m, 2H), 7.35-7.40 (m, 1H), 7.16-7.19 (dd, 1H), 6.77-6.81 (m, 1H), 5.23 (s, 2H).

### Step B: Methyl 2-((2-(benzyloxy)-4-fluorophenyl)amino)-5-(trifluoromethyl)benzoate 15b

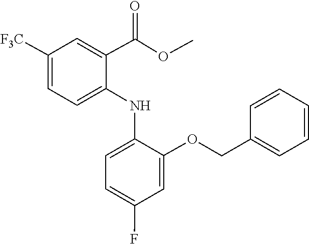 [SMILES: COC(=O)c1cc(C(F)(F)F)ccc1Nc1ccc(F)cc1OCc1ccccc1]

A flask containing methyl 2-amino-5-(trifluoromethyl)benzoate (8.75 g, 39.9 mmol), 2-(benzyloxy)-1-bromo-4-fluorobenzene (13.5 g, 47.9 mmol) and Cs₂CO₃ (19.5 g, 59.9 mmol) in toluene (170 mL) was purged with N₂ for 20 min, to which was added BINAP (2.49 g, 3.99 mmol) and Pd₂(dba)₃ (1.83 g, 1.99 mmol), the reaction purged with N₂ an additional 5 min, then heated at 100° C. for 16 h. The reaction mixture was allowed to cool to ambient temperature, filtered through celite, washed with EtOAc (450 mL), and the filtrate evaporated under reduced pressure. The crude residue was preabsorbed onto silica gel and purified by silica gel flash column chromatography (340 g) eluting with 0-1% EtOAc in pet-ether gradient. Product fractions were combined and evaporated under reduced pressure to afford methyl 2-((2-(benzyloxy)-4-fluorophenyl)amino)-5-(trifluoromethyl)benzoate 15b (11.1 g, 64% yield) as a yellow gum. HPLC/MS 1.48 min (C), [M+H]⁺ 420.0. ¹H NMR (DMSO-d₆, 400 MHz) δ 9.53 (s, 1H), 8.11-8.12 (d, 1H), 7.64-7.67 (m, 1H), 7.41-7.44 (m, 1H), 7.28-7.31 (m, 5H), 7.18-7.21 (m, 1H), 6.98-7.01 (d, 1H), 6.84-6.89 (m, 1H), 5.18 (s, 2H), 3.88 (s, 3H).

### Step C: Methyl 2-((4-fluoro-2-(hydroxyphenyl)amino)-5-(trifluoromethyl)benzoate 15c

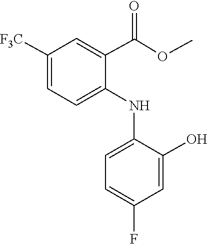 [SMILES: COC(=O)c1cc(C(F)(F)F)ccc1Nc1ccc(F)cc1O]

To a solution of methyl 2-((2-(benzyloxy)-4-fluorophenyl)amino)-5-(trifluoromethyl)benzoate (11.1 g, 26.5 mmol) in EtOH (140 mL) was added Pd—C(10% WET) (5.63 g, 5.29 mmol) and the reaction mixture was stirred under H₂ atmosphere for 9 h. The reaction mixture was filtered through celite, washed with MeOH (750 mL) and the filtrate was concentrated under reduced pressure to afford methyl 2-((4-fluoro-2-(hydroxyphenyl)amino)-5-(trifluoromethyl)benzoate 15c (8.45 g, 93% yield) as a brown gum. HPLC/MS 1.18 min (C), [M+H]⁺ 330.1. ¹H NMR (DMSO-d₆, 400 MHz) δ 10.35 (s, 1H), 9.38 (s, 1H), 8.11-8.12 (d, 1H), 7.63-7.66 (m, 1H), 7.28-7.32 (m, 1H), 6.88-6.91 (d, 1H), 6.76-6.79 (m, 1H), 6.68-6.73 (m, 1H), 3.90 (s, 3H).

### Step D: Methyl 2-((2-(2-((tert-butoxycarbonyl)amino)ethoxy)-4-fluorophenyl)amino)-5-(trifluoromethyl) benzoate 15d

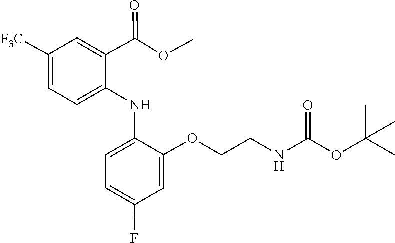 [SMILES: COC(=O)c1cc(C(F)(F)F)ccc1Nc1ccc(F)cc1OCCNC(=O)OC(C)(C)C]

To a solution of methyl 2-((4-fluoro-2-hydroxyphenyl)amino)-5-(trifluoromethyl)benzoate (8.40 g, 25.5 mmol) in THF (80 mL), purged with N₂, was added tert-butyl (2-hydroxyethyl)carbamate (8.23 g, 51.0 mmol) and triphenylphosphine (10.0 g, 38.3 mmol). A solution of DEAD (6.06 mL, 38.3 mmol) in THF (10 mL) was added to the reaction mixture and the reaction stirred at room temperature for 16 h. The reaction mixture was quenched with H₂O (200 mL), extracted with EtOAc (2×150 mL), and the combined organic extracts were washed with brine, dried over Na₂SO₄, filtered and evaporated under reduced pressure. The crude residue was preabsorbed onto silica gel and purified by silica gel flash column chromatography (220 g), eluting with a 0-20% EtOAc in pet-ether gradient. Product fractions were combined and evaporated under reduced pressure to afford methyl 2-((2-(2-((tert-butoxycarbonyl) amino)ethoxy)-4-fluorophenyl)amino)-5-(trifluoromethyl) benzoate 15d (10.5 g, 87% yield) as a yellow solid. HPLC/MS 1.42 min (C), [M+H]⁺ 471.0. ¹H NMR (DMSO-d₆, 400 MHz) δ 9.52 (s, 1H), 8.12-8.13 (d, 1H), 7.64-7.67 (dd, 1H), 7.39-7.42 (m, 1H), 7.03-7.13 (m, 2H), 6.78-6.86 (m, 2H), 3.90 (s, 3H), 4.03-4.06 (m, 2H), 3.90 (s, 3H), 3.22-3.34 (m, 2H), 1.25-1.40 (s, 9H).

### Step E: 2-((2-(2-((tert-Butoxycarbonyl)amino)ethoxy)-4-fluorophenyl)amino)-5-(trifluoromethyl) benzoic acid 15e

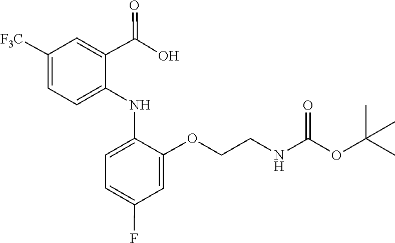 [SMILES: CC(C)(C)OC(=O)NCCOc1cc(F)ccc1Nc1ccc(C(F)(F)F)cc1C(=O)O]

Following the procedure outlined in Example 10, Step B, using a THF/MeOH/H₂O solvent mixture and stirring the reaction mixture at 35° C. for 3 h, 2-((2-(2-((tert-butoxycarbonyl)amino)ethoxy)-4-fluorophenyl)amino)-5-(trifluoromethyl) benzoic acid 15e (9.36 g, 92% yield) was prepared as an off-white solid. HPLC/MS 1.28 min (C), [M+H]⁺ 459.3. ¹H-NMR (DMSO-d₆, 400 MHz) δ 12.52 (s, 1H), 8.19-8.20 (d, 1H), 7.37-7.40 (m, 2H), 7.15-7.19 (m, 2H), 6.94-6.97 (m, 1H), 6.72-6.77 (m, 1H), 3.96-3.98 (m, 2H), 3.30-3.35 (m, 2H), 1.34 (s, 9H).

### Step F: tert-Butyl (2-(2-((2-((2-bromo-6-methoxypyridin-3-yl)carbamoyl)-4-(trifluoro-methyl)phenyl) amino)-5-fluorophenoxy)ethyl)carbamate 15f

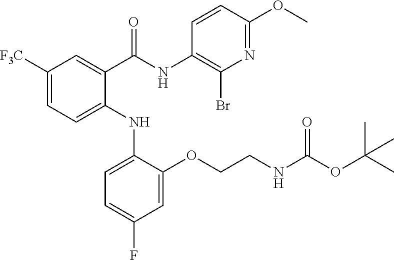 [SMILES: COc1ccc(NC(=O)c2cc(C(F)(F)F)ccc2Nc2ccc(F)cc2OCCNC(=O)OC(C)(C)C)c(Br)n1]

Following the procedure outlined in Example 11, Step D, stirring the reaction mixture at 80° C. for 16 h, tert-butyl (2-(2-((2-((2-bromo-6-methoxypyridin-3-yl)carbamoyl)-4-(trifluoromethyl)phenyl)amino)-5-fluorophenoxy)ethyl)carbamate 15f (3.94 g, 30% yield) was prepared as a brown solid. HPLC/MS 1.44 min (C), [M+H]⁺ 641.0. ¹H-NMR (DMSO-d₆, 400 MHz) δ 10.44 (s, 1H), 9.77 (s, 1H), 8.23-8.24 (d, 1H), 7.84-7.86 (d, 1H), 7.63-7.66 (m, 1H), 7.37-7.41 (m, 1H), 7.16-7.18 (m, 1H), 7.05-7.08 (m, 1H), 6.94-6.96 (d, 1H), 6.81-6.84 (m, 1H), 6.71-6.79 (m, 1H), 4.01-4.04 (m, 1H), 3.88 (s, 3H), 3.22-3.34 (m, 2H), 1.99 (s, 9H).

### Step G: tert-Butyl (2-(2-(3-(2-bromo-6-methoxypyridin-3-yl)-4-oxo-6-(trifluoromethyl)-3,4-dihydro quinazolin-1(2H)-yl)-5-fluorophenoxy)ethyl)carbamate 152

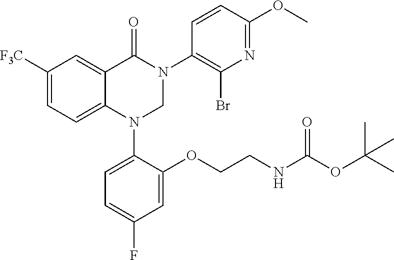 [SMILES: COc1ccc(N2CN(c3ccc(F)cc3OCCNC(=O)OC(C)(C)C)c3ccc(C(F)(F)F)cc3C2=O)c(Br)n1]

Following the procedure outlined in Example 11, Step E, stirring the reaction mixture at 80° C. for 16 h, tert-butyl (2-(2-(3-(2-bromo-6-methoxypyridin-3-yl)-4-oxo-6-(trifluoromethyl)-3,4-dihydroquinazolin-1(2H)-yl)-5-fluorophenoxy)ethyl)carbamate 15g (1.5 g, 36% yield) was prepared as a yellow solid. HPLC/MS 1.39 min (C), [M-Boc]⁺ 554.6.

### Step H: 1-(2-(2-Aminoethoxy)-4-fluorophenyl)-3-(2-bromo-6-methoxypyridin-3-yl)-6-(trifluoromethyl)-2,3-dihydroquinazolin-4(1H)-one, trifluoroacetate salt 15h

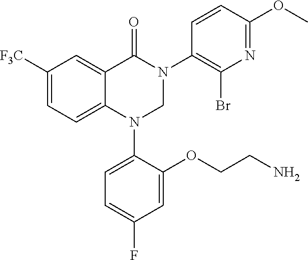 [SMILES: COc1ccc(N2CN(c3ccc(F)cc3OCCN)c3ccc(C(F)(F)F)cc3C2=O)c(Br)n1]

Following the procedure outlined in Example 11, Step F, using TFA/DCM in place of HCl/dioxane and stirring at room temperature for 1 h, 1-(2-(2-aminoethoxy)-4-fluorophenyl)-3-(2-bromo-6-methoxypyridin-3-yl)-6-(trifluoromethyl)-2,3-dihydroquinazolin-4(1H)-one 15h (1.54 g, >100% yield) was prepared as a brown solid. HPLC/MS 0.82 min (C), [M+H]⁺ 554.8. ¹H-NMR (DMSO-d₆, 400 MHz) δ 8.10 (s, 1H), 7.83-7.89 (m, 3H), 7.66-7.68 (m, 1H), 7.43-7.47 (m, 1H), 7.16-7.25 (m, 2H), 6.92-6.98 (m, 2H), 6.60-6.62 (d, 1H), 5.50 (br s, 1H), 4.98 (br s, 1H), 4.21-4.24 (m, 2H), 3.87 (s, 3H), 3.15-3.22 (m, 2H).

### Step I: 8-Fluoro-15-methoxy-2-(trifluoromethyl)-12,13-dihydro-11H,19H-5,18-methano-dibenzo[i,j]pyrido[2,3-e][1]oxa[4,7,11]triazacyclotridecin-19-one 15i

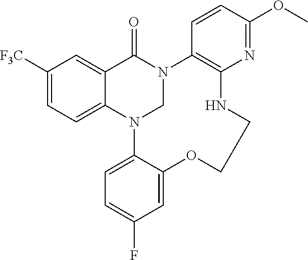 [SMILES: COc1ccc2c(n1)NCCOc1cc(F)ccc1N1CN2C(=O)c2cc(C(F)(F)F)ccc21]

Following the procedure outlined in Example 11, Step G, stirring the reaction mixture at 90° C. for 16 h, 8-fluoro-15-methoxy-2-(trifluoromethyl)-12,13-dihydro-11H,19H-5,18-methanodibenzo[i,j]pyrido[2,3-e][1]oxa[4,7,11]triazacyclotridecin-19-one 15i (200 mg, 30% yield) was prepared as a yellow solid. HPLC/MS 1.25 min (C), [M+H]⁺ 474.8. ¹H-NMR (DMSO-d₆, 400 MHz) δ 8.11-8.12 (s, 1H), 7.68-7.71 (m, 1H), 7.55-7.59 (m, 1H), 7.48-7.50 (d, 1H), 7.42-7.44 (m, 1H), 7.05-7.10 (m, 1H), 6.73-6.75 (d, 1H), 6.11-6.13 (d, 1H), 5.91-5.94 (m, 1H), 5.33-5.36 (m, 1H), 4.48-4.53 (m, 2H), 3.92-3.97 (m, 2H), 3.80 (s, 3H).

### 8-Fluoro-2-(trifluoromethyl)-12,13-dihydro-11H,19H-5,18-methanodibenzo[i,j]pyrido-[2,3-e][1]oxa[4,7,11]triazacyclotridecine-15.19(14H)-dione Example 15

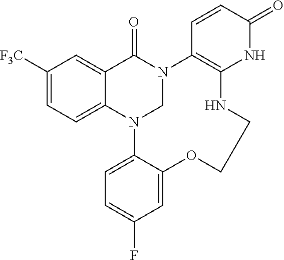 [SMILES: O=C1c2cc(C(F)(F)F)ccc2N2CN1c1ccc(=O)[nH]c1NCCOc1cc(F)ccc12]

Following the procedure outlined in Example 11, Step H, stirring the reaction mixture at 100° C. for 5 h, 8-fluoro-2-(trifluoromethyl)-12,13-dihydro-11H,19H-5,18-methanodibenzo[i,j]pyrido[2,3-e][1]oxa[4,7,11]triazacyclotridecine-15,19(14H)-dione (22 mg) was prepared as an off-white solid. HPLC/MS 2.69 min (D), [M+H]⁺ 461.1. ¹H-NMR (DMSO-d₆, 400 MHz) δ 10.79 (brs, 1H), 8.11 (d, J=1.60 Hz, 1H), 7.68 (dd, J=2.00, 8.80 Hz, 1H), 7.57 (dd, J=6.40, 8.80 Hz, 1H), 7.43-7.36 (m, 2H), 7.06 (td, J=2.8 Hz, 8.4 Hz, 1H), 6.76 (d, J=8.80 Hz, 1H), 5.87 (d, J=7.60 Hz, 1H), 5.65 (s, 1H), 5.34 (d, J=11.60 Hz, 1H), 4.49-4.45 (m, 2H), 4.00-3.95 (m, 1H), 3.85-3.79 (m, 1H), 3.40-3.34 (m, 1H).

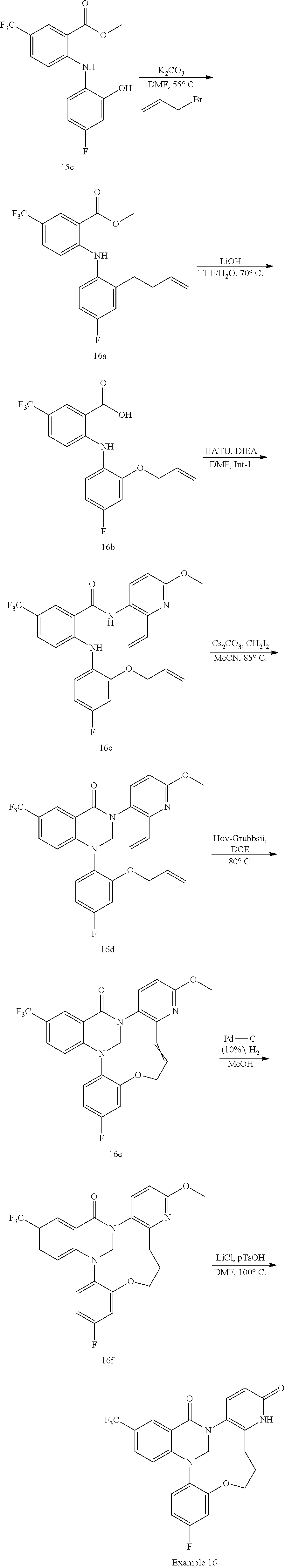

### Example 16

### 8-Fluoro-2-(trifluoromethyl)-12,13-dihydro-11H,19H-5,18-methanodibenzo[b,e]pyrido[3,2-i][1]oxa[4,8]diazacyclotridecine-15,19(14H)-dione

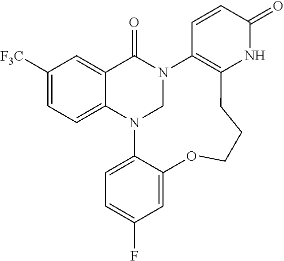 [SMILES: O=C1c2cc(C(F)(F)F)ccc2N2CN1c1ccc(=O)[nH]c1CCCOc1cc(F)ccc12]

### Step A: Methyl 2-((2-(allyloxy)-4-fluorophenyl)amino)-5-(trifluoromethyl)benzoate 16a

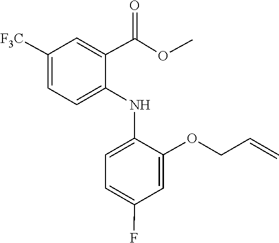 [SMILES: C=CCOc1cc(F)ccc1Nc1ccc(C(F)(F)F)cc1C(=O)OC]

To a solution of methyl 2-((4-fluoro-2-hydroxyphenyl)amino)-5-(trifluoromethyl)benzoate (7.00 g, 21.3 mmol) in acetone (100 mL), under N₂, was added K₂CO₃ (5.88 g, 42.5 mmol) portion-wise, the suspension stirred for 10 min, to which was added allyl bromide (3.68 mL, 42.5 mmol) and the reaction mixture was stirred at 55° C. for 2 h. The reaction mixture was allowed to cool to ambient temperature and concentrated under reduced pressure. The residue was diluted with H₂O (200 mL), extracted with EtOAc (2×200 mL), the combined extracts washed with brine (200 mL), dried over Na₂SO₄, filtered and evaporated under reduced pressure to afford methyl 2-((2-(2-(allyloxy)-4-fluorophenyl)amino)-5-(trifluoromethyl) benzoate 16a (8.0 g, 99% yield) as a yellow solid. HPLC/MS 1.45 min (C), [M+H]⁺ 370.0. ¹H-NMR (DMSO-d₆, 400 MHz) δ 9.52 (s, 1H), 8.12 (s, 1H), 7.64-7.67 (dd, 1H), 7.40-7.43 (m, 1H), 7.08-7.11 (m, 1H), 7.00-7.02 (d, 1H), 6.82-6.87 (m, 1H), 5.91-5.98 (m, 1H), 5.28-5.32 (m, 1H), 4.63-4.65 (m, 2H), 3.90 (s, 3H).

### Step B: 2-((2-((Allyloxy)-4-fluorophenyl)amino)-5-(trifluoromethyl)benzoic acid 16b

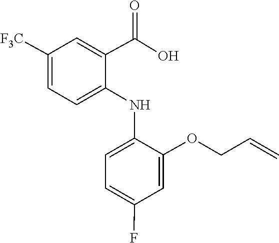 [SMILES: C=CCOc1cc(F)ccc1Nc1ccc(C(F)(F)F)cc1C(=O)O]

Following the procedure outlined in Example 15, Step E, stirring the reaction mixture at 60° C. for 4 h, 2-((2-((allyloxy)-4-fluorophenyl)amino)-5-(trifluoromethyl)benzoic acid 16b (7.5 g, 96% yield) was prepared as a yellow solid. HPLC/MS 1.36 min (C), [M+H]⁺ 356.0. ¹H-NMR (DMSO-d₆, 400 MHz) δ 13.5-13.8 (brs, 1H), 9.91 (s, 1H), 8.12 (s, 1H), 7.62-7.65 (m, 1H), 7.41-7.45 (m, 1H), 7.03-7.10 (m, 2H), 6.82-6.87 (m, 1H), 5.93-6.00 (m, 1H), 5.33-5.38 (m, 1H), 5.19-5.22 (m, 1H), 4.63-4.64 (d, 2H).

### Step C: 2-((2-((Allyloxy)-4-fluorophenyl)amino)-N-(6-methoxy-2-vinylpyridine-3-yl)-5-(trifluoromethyl) benzamide 16c

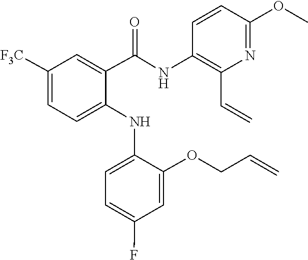 [SMILES: C=CCOc1cc(F)ccc1Nc1ccc(C(F)(F)F)cc1C(=O)Nc1ccc(OC)nc1C=C]

Following the procedure outlined in Example 15, Step F and substituting Int-1 for 2-bromo-6-methoxypyridin-3-amine, stirring the reaction mixture at room temperature for 4 h, 2-((2-((allyloxy)-4-fluorophenyl)amino)-N-(6-methoxy-2-vinylpyridin-3-yl)-5-(trifluoromethyl)benzamide 16c (7.5 g, 96% yield) was prepared as a yellow solid. HPLC/MS 1.44 min (C), [M+H]⁺ 487.8. ¹H-NMR (DMSO-d₆, 400 MHz) δ 10.3 (s, 1H), 9.81 (s, 1H), 8.26 (s, 1H), 7.61-7.68 (m, 2H), 7.37-7.41 (m, 1H), 7.03-7.06 (m, 1H), 6.87-6.91 (m, 1H), 6.80-6.82 (m, 2H), 6.36-6.41 (m, 1H), 5.88-5.99 (m, 1H), 5.47-5.50 (m, 1H), 5.33-5.38 (m, 1H), 5.19-5.19 (m, 1H), 4.61-4.62 (m, 2H), 3.92 (s, 3H).

### Step D: 1-(2-(Allyloxy)-4-fluorophenyl)-3-(6-methoxy-2-vinylpyridine-3-yl)-6-(trifluoro-methyl)-2,3-dihydro quinazolin-4-(11H)-one 16d

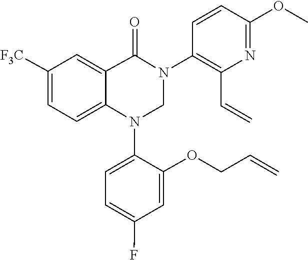 [SMILES: C=CCOc1cc(F)ccc1N1CN(c2ccc(OC)nc2C=C)C(=O)c2cc(C(F)(F)F)ccc21]

Following the procedure outlined in Example 15, Step G, stirring the reaction mixture at 80° C. for 4 h, 1-(2-(allyloxy)-4-fluorophenyl)-3-(6-methoxy-2-vinylpyridin-3-yl)-6-(trifluoromethyl)-2,3-dihydroquinazolin-4-(1H)-one 16d (4.0 g, 47% yield) was prepared as a yellow semi-solid. HPLC/MS 1.40 min (C), [M+H]⁺ 499.8. ¹H-NMR (DMSO-d₆, 400 MHz) δ 8.08 (s, 1H), 7.71-7.73 (m, 1H), 7.65-7.67 (m, 1H), 7.46-7.47 (m, 1H), 7.14-7.17 (m, 1H), 6.82-6.94 (m, 3H), 6.53-6.55 (d, 1H), 6.34-6.39 (m, 1H), 5.88-5.99 (m, 1H), 5.44-5.46 (m, 2H), 5.13-5.16 (m, 2H), 4.83-4.86 (m, 1H), 4.62-4.63 (m, 2H), 3.90 (s, 3H).

### Step E: 8-Fluoro-15-methoxy-2-(trifluoromethyl)-11H,19H-5,18-methanodibenzo[b,e]-pyrido[3,2-i][1]oxa [4,8]diazacyclotridecin-19-one 16e

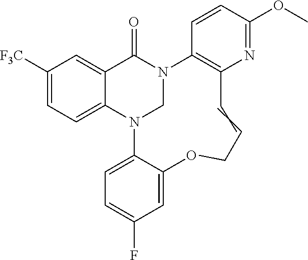 [SMILES: COc1ccc2c(n1)C=CCOc1cc(F)ccc1N1CN2C(=O)c2cc(C(F)(F)F)ccc21]

Following the procedure outlined in Example 1, Step E, stirring the reaction mixture at 40° C. for 16 h, 8-fluoro-15-methoxy-2-(trifluoromethyl)-11H,19H-5,18-methanodibenzo[b,e]pyrido[3,2-i][1]oxa[4,8]diazacyclotridecin-19-one 16e was isolated (150 mg, 7% yield) as an off-white solid. HPLC/MS 1.26 min (C), [M+H]⁺ 471.6. ¹H-NMR (DMSO-d₆, 400 MHz) δ 8.10 (s, 1H), 7.76-7.78 (d, 1H), 7.63-7.66 (dd, 1H), 7.43-7.47 (m, 1H), 7.33-7.36 (m, 1H), 7.18-7.22 (m, 1H), 6.78-6.80 (d, 1H), 6.43-6.47 (m, 1H), 6.20-6.21 (m, 1H), 5.58-5.61 (d, 1H), 4.85-4.89 (m, 1H), 4.78-4.81 (d, 1H), 4.22-4.28 (m, 1H), 3.80 (s, 3H).

### Step F: 8-Fluoro-15-methoxy-2-(trifluoromethyl)-12,13-dihydro-11H,19H-5,18-methanodibenzo[b,e]pyrido[3,2-j][1]oxa[4,8]diazacyclotridecin-19-one 16f

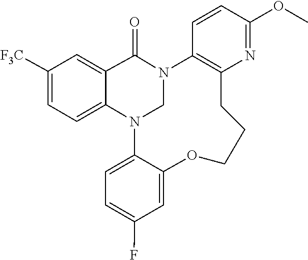 [SMILES: COc1ccc2c(n1)CCCOc1cc(F)ccc1N1CN2C(=O)c2cc(C(F)(F)F)ccc21]

Following the procedure outlined in Example 1, Step G, stirring with Pd(OH)₂ (20% wt on carbon) for 1 h, 8-fluoro-15-methoxy-2-(trifluoromethyl)-12,13-dihydro-11H,19H-5,18-methanodibenzo[b,e]pyrido[3,2-i][1]oxa[4,8]diazacyclotridecin-19-one 16f was isolated (150 mg, 7% yield) as an off-white solid. HPLC/MS 1.27 min (C), [M+H]⁺ 473.8. ¹H-NMR (DMSO-d₆, 400 MHz) δ 8.11 (s, 1H), 7.65-7.68 (m, 2H), 7.56-7.60 (m, 1H), 7.34-7.47 (m, 1H), 6.98-7.02 (m, 1H), 6.74-6.77 (m, 2H), 5.56-5.59 (m, 1H), 4.66-4.69 (m, 1H), 4.30-4.32 (m, 1H), 3.86 (s, 3H), 3.60-3.61 (m, 1H), 2.90-3.02 (m, 1H), 2.32-2.36 (m, 1H), 1.99-2.18 (m, 2H).

### Step G: 8-Fluoro-2-(trifluoromethyl)-12,13-dihydro-11H,19H-5,18-methanodibenzo-[b,e]pyrido[3,2-i][1]oxa[4,8]diazacyclotridecin-15,19(14H)-dione Example 16

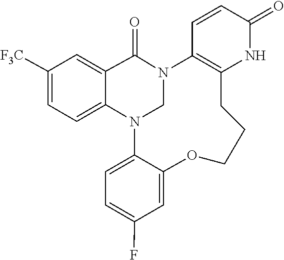 [SMILES: O=C1c2cc(C(F)(F)F)ccc2N2CN1c1ccc(=O)[nH]c1CCCOc1cc(F)ccc12]

Following the procedure outlined in Example 1, Step H, stirring at 100° C. for 2 h, 8-fluoro-2-(trifluoromethyl)-12,13-dihydro-11H,19H-5,18-methanodibenzo[b,e]pyrido[3,2-i][1]oxa[4,8]diazacyclo tridecin-15,19(14H)-dione was isolated (18 mg, 53% yield) as an off-white solid. HPLC/MS 1.00 min (C), [M+H]⁺ 459.8. ¹H-NMR (DMSO-d₆, 400 MHz) δ 11.85 (s, 1H), 8.09 (d, J=2.00 Hz, 1H), 7.66 (dd, J=2.00, 9.00 Hz, 1H), 7.58 (dd, J=6.40, 8.80 Hz, 1H), 7.44-7.36 (m, 2H), 7.04-6.99 (m, 1H), 6.75 (d, J=8.80 Hz, 1H), 6.24 (d, J=9.60 Hz, 1H), 5.49 (d, J=11.60 Hz, 1H), 4.67 (d, J=11.60 Hz, 1H), 4.34-4.29 (m, 1H), 3.84-3.79 (m, 1H), 2.76-2.67 (m, 1H), 2.34-2.32 (m, 1H), 2.00-1.99 (m, 1H), 1.99-1.81 (m, 1H).

### Example 17

### 8-(Trifluoromethoxy)-2-(trifluoromethyl)-10,11,12,13-tetahydro-19H,19H-5,18-methano-dibenzo[l]pyrido[2,3-b:3′,2′-f][1,5]diazacyclotridecine-15,19(14H)-dione

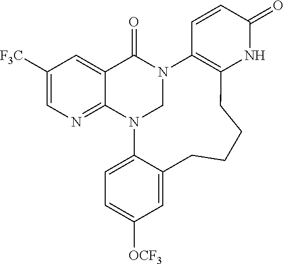 [SMILES: O=C1c2cc(C(F)(F)F)cnc2N2CN1c1ccc(=O)[nH]c1CCCCc1cc(OC(F)(F)F)ccc12]

### Step A: Methyl 2-((2-bromo-4-(trifluoromethoxy)phenyl)amino)-5-(trifluoromethyl)-nicotinate 17a

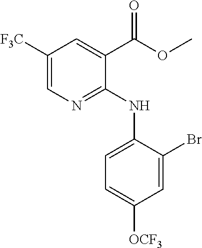 [SMILES: COC(=O)c1cc(C(F)(F)F)cnc1Nc1ccc(OC(F)(F)F)cc1Br]

Following the procedure outlined in Example 3, Step A, substituting Int-2a with Int-2b, 2-bromo-4-fluoroaniline with 2-bromo-4-(trifluoromethoxy)aniline, and BINAP with Xantphos, and stirring the reaction mixture at 85° C. for 16 h, methyl 2-((2-bromo-4-(trifluoromethoxy)phenyl)amino)-5-(trifluoromethyl)nicotinate 17a (21.0 g, 51% yield), in 68% purity, was prepared as a yellow solid. HPLC/MS 1.55 min (C), [M+H]⁺ 461.0. ¹H-NMR (DMSO-d₆, 400 MHz) δ 10.58 (s, 1H), 8.79 (s, 1H), 8.41-8.43 (m, 1H), 7.84 (s, 1H), 7.48-7.50 (m, 1H), 6.82-6.84 (d, 1H).

### Step B: 2-((2-Bromo-4-(trifluoromethoxy)phenyl)amino)-5-(trifluoromethyl)nicotinic acid 17b

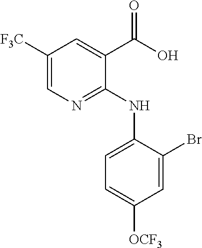 [SMILES: O=C(O)c1cc(C(F)(F)F)cnc1Nc1ccc(OC(F)(F)F)cc1Br]

Following the procedure outlined in Example 3, Step B, stirring the reaction mixture at room temperature for 12 h, 2-((2-bromo-4-(trifluoromethoxy)phenyl)amino)-5-(trifluoromethyl)nicotinic acid 17b (10.5 g, 75% yield), was prepared as a yellow solid. HPLC/MS 1.64 min (C), [M+H]⁺ 445.2. ¹H-NMR (DMSO-d₆, 400 MHz) δ 10.97 (s, 1H), 8.75-8.76 (d, 1H), 8.45-8.47 (d, 2H), 7.82 (s, 1H), 7.47-7.50 (m, 1H).

### Step C: 2-((2-Bromo-4-(trifluoromethoxy)phenyl)amino)-N-(2-bromo-6-methoxypyridin-3-yl)-5-(trifluoromethyl)nicotinamide 17c

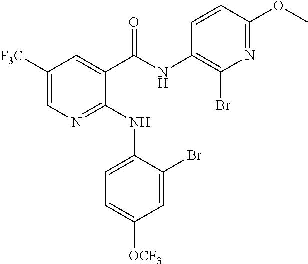 [SMILES: COc1ccc(NC(=O)c2cc(C(F)(F)F)cnc2Nc2ccc(OC(F)(F)F)cc2Br)c(Br)n1]

Following the procedure outlined in Example 3, Step C, stirring the reaction mixture at room temperature for 12 h, 2-((2-bromo-4-(trifluoromethoxy)phenyl)amino)-N-(2-bromo-6-methoxypyridin-3-yl)-5-(trifluoromethyl)nicotinamide 17c (10.2 g, 49% yield), in 64% purity, was prepared as a brown solid. HPLC/MS 1.57 min (C), [M+H]⁺ 631.0.

### Step D: 1-((2-Bromo-4-(trifluoromethoxy)phenyl)-3-(2-bromo-6-methoxypyridin-3-yl)-6-(trifluoromethyl)-2,3-dihydropyrido[2,3-d]pyrimidin-4(11H)-one 17d

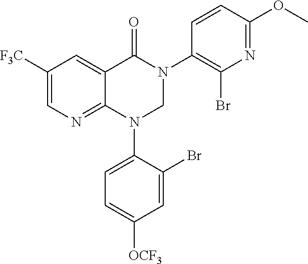 [SMILES: COc1ccc(N2CN(c3ccc(OC(F)(F)F)cc3Br)c3ncc(C(F)(F)F)cc3C2=O)c(Br)n1]

Follow the procedure outlined in Example 3, Step D, stirring the reaction mixture at 120° C. for 2 h under microwave irradiation, 1-((2-bromo-4-(trifluoromethoxy)phenyl)-3-(2-(bromo-6-methoxypyridin-3-yl)-6-(trifluoromethyl)-2,3-dihydropyrido[2,3-d]pyrimidin-4(1H)n-one 17d (580 mg, 19% yield), in <80% purity, was prepared as a brown gum. HPLC/MS 1.48 min (C), [M+H]⁺ 643.2.

### Step E: 1-(2-Allyl-4-(trifluoromethoxy)phenyl)-3-(2-allyl-6-methoxypyridin-3-yl)-6-(trifluoromethyl)-2,3-dihydropyrido[2,3-d]pyrimidin-4(1H)-one 17e F₃C N N N˜NJ OCF₃

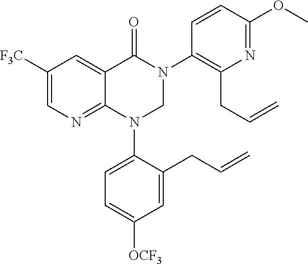 [SMILES: C=CCc1cc(OC(F)(F)F)ccc1N1CN(c2ccc(OC)nc2CC=C)C(=O)c2cc(C(F)(F)F)cnc21]

Following the procedure outlined in Example 3, Step E, stirring the reaction mixture at 130° C. for 12 h, 1-(2-allyl-4-(trifluoromethoxy)phenyl)-3-(2-allyl-6-methoxypyridin-3-yl)-6-(trifluoromethyl)-2,3-dihydro pyrido[2,3-d]pyrimidin-4(1H)n-one 17e (1.05 g, 93% yield), was prepared as a brown gum. HPLC/MS 1.46/1.48 min (C), [M+H]⁺ 565.2.

### Step F: 15-Methoxy-8-(trifluoromethoxy)-2-(trifluoromethyl)-10,13-dihydro-19H-5,18-methanobenzo[l]dipyrido[2,3-b:3′,2′-f]diazacyclotridecin-19-one 17f

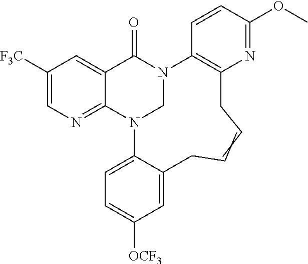 [SMILES: COc1ccc2c(n1)CC=CCc1cc(OC(F)(F)F)ccc1N1CN2C(=O)c2cc(C(F)(F)F)cnc21]

Following the procedure outlined in Example 3, Step F, stirring the reaction mixture at 80° C. for 28 h, 15-methoxy-8-(trifluoromethoxy)-2-(trifluoromethyl)-10,13-dihydro-19H-5,18-methanobenzo[l]dipyrido[2,3-b:3′,2′-f]diazacyclotridecin-19-one 17f (340 mg, 37% yield), in 52% purity, was prepared as a green solid. HPLC/MS 1.39 min (C), [M+H]⁺ 537.2.

### Step G: 15-Methoxy-8-(trifluoromethoxy)-2-(trifluoromethyl)-10,11,12,13-tetrahydro-19H-5,18-methanobenzo[l]dipyrido[2,3-b:3′,2′-f]diazacyclotridecin-19-one 172

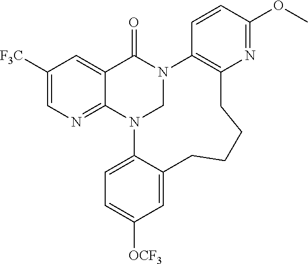 [SMILES: COc1ccc2c(n1)CCCCc1cc(OC(F)(F)F)ccc1N1CN2C(=O)c2cc(C(F)(F)F)cnc21]

To a solution of 15-methoxy-8-(trifluoromethoxy)-2-(trifluoromethyl)-10,11,12,13-tetrahydro-19H-5,18-methanobenzo[l]dipyrido[2,3-b:3′,2′-f]diazacyclotridecin-19-one (120 mg, 0.116 mmol) in DCM (10 mL) was added (1,5-cyclooctadiene)(pyridine)tricyclohexylphosphine)iridium(I)hexafluorophosphate (18.7 mg, 0.023 mmol) and the reaction mixture was stirred under H₂ (1 kg/cm² pressure) at room temperature for 12 h. The reaction mixture was evaporated under reduced pressure, the residue dissolved in DCM, adsorbed onto silica gel and purified by silica gel flash column chromatography (25 g), eluting with 22% EtOAc-pet ether. Product fractions were combined, the solvent evaporated under reduced pressure, and dried under vacuo to afford 15-methoxy-8-(trifluoromethoxy)-2-(trifluoromethyl)-10,11,12,13-tetrahydro-19H-5,18-methanobenzo[l]dipyrido[2,3-b:3′,2′-f]diazacyclotridecin-19-one 17g (61 mg, 94% yield) as a brown gum. HPLC/MS 1.36 min (C), [M+H]⁺ 539.2. ¹H NMR (DMSO-d₆, 400 MHz) δ 8.65 (s, 1H), 8.40 (s, 1H), 7.66-7.68 (d, 1H), 7.45-7.50 (m, 2H), 7.32-7.33 (m, 1H), 6.74-6.76 (d, 1H), 5.78-5.81 (m, 1H), 4.89-4.92 (d, 1H), 4.00-4.06 (m, 1H), 3.84 (s, 3H), 2.81-2.85 (m, 1H), 2.72-2.76 (m, 1H), 1.53-1.68 (m, 3H).

### Step H: 8-(Trifluoromethoxy)-2-(trifluoromethyl)-10,11,12,13-tetahydro-19H-5,18-methanodibenzo [l]pyrido[2,3-b:3′,2′-f][1,5]diazacyclotridecine-15,19(14H)-dione Example 17

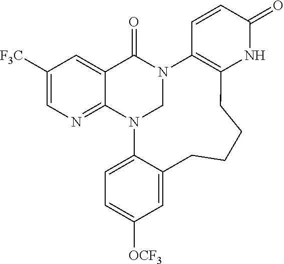 [SMILES: O=C1c2cc(C(F)(F)F)cnc2N2CN1c1ccc(=O)[nH]c1CCCCc1cc(OC(F)(F)F)ccc12]

Following the procedure outlined in Example 3, Step G, stirring the reaction mixture at 100° C. for 2 h, 8-(trifluoromethoxy)-2-(trifluoromethyl)-10,11,12,13-tetahydro-19H-5,18-methanodibenzo[l]pyrido[2,3-b:3′,2′-f][1,5]diazacyclotridecine-15,19(14H)-dione (25 mg, 41% yield), was prepared as an off-white solid. HPLC/MS 1.04 min (C), [M+H]⁺ 525.2. ¹H NMR (DMSO-d₆, 400 MHz) δ 11.85 (bs, 1H), 8.63 (s, 1H), 8.52 (bs, 1H), 8.38 (s, 1H), 7.55-7.30 (m, 4H), 6.23 (d, J=9.6 Hz, 1H), 5.70 (d, J=11.6 Hz, 1H), 4.89 (d, J=11.6 Hz, 1H), 2.73-2.68 (m, 1H), 2.62-2.58 (m, 1H), 2.51-2.42 (m, 1H), 2.28-2.23 (m, 1H), 1.72-1.61 (m, 2H), 1.52-1.42 (m, 2H).

### Example 18

3⁴-Fluoro-2⁷-(trifluoromethyl)-1¹,1²,2¹,2²,2³,2⁴-hexahydro-4-oxa-2(3,1)-quinazolina-1(5,6)-pyridina-3(1,2)-benzenacyclooctaphane-1²,2⁴-dione

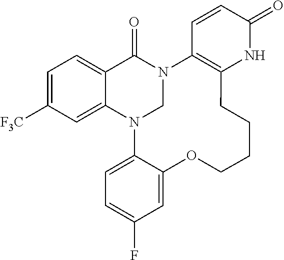 [SMILES: O=C1c2ccc(C(F)(F)F)cc2N2CN1c1ccc(=O)[nH]c1CCCCOc1cc(F)ccc12]

### Step A: Methyl 2-((2-(benzyloxy)-4-fluorophenyl)amino)-4-(trifluoromethyl)benzoate 18a

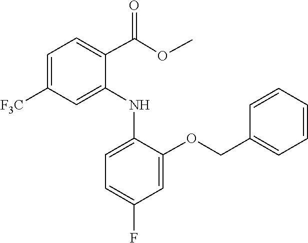 [SMILES: COC(=O)c1ccc(C(F)(F)F)cc1Nc1ccc(F)cc1OCc1ccccc1]

Following the procedure outlined in Example 15, Step B, substituting methyl 2-amino-4-(trifluoromethyl)benzoate for methyl 2-amino-5-(trifluoromethyl)benzoate, and stirring the reaction mixture at 90° C. for 72 h, methyl 2-((2-(benzyloxy)-4-fluorophenyl)amino)-4-(trifluoromethyl)benzoate 18a (6.25 g, 59% yield) was prepared as a light yellow solid. HPLC/MS 1.55 min (A), [M+H]⁺ 420.1. ¹H NMR (DMSO-d₆, 400 MHz) δ 9.3-9.5 (m, 1H), 8.07 (d, 1H, J=8.3 Hz), 7.42 (dd, 1H, J=6.4, 8.8 Hz), 7.2-7.3 (m, 5H), 7.1-7.2 (m, 1H), 7.0-7.1 (m, 2H), 6.88 (dt, 1H, J=2.9, 8.6 Hz), 5.19 (s, 2H), 3.89 (s, 3H).

### Step B: Methyl 2-((4-fluoro-2-hydroxvphenyl)amino)-4-(trifluoromethyl)benzoate 18b

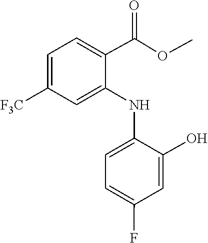 [SMILES: COC(=O)c1ccc(C(F)(F)F)cc1Nc1ccc(F)cc1O]

Following the procedure outlined in Example 15, Step C, and stirring the reaction mixture at room temperature for 24 h, methyl 2-((4-fluoro-2-hydroxyphenyl) amino)-4-(trifluoromethyl)benzoate 18b (4.95 g, 98% yield) was prepared as a light yellow solid. HPLC/MS 1.25 min (A), [M+H]⁺ 330.0. ¹H NMR (DMSO-d₆, 400 MHz) δ 10.3-10.5 (m, 1H), 9.19 (s, 1H), 8.07 (d, 1H, J=8.3 Hz), 7.30 (dd, 1H, J=6.1, 8.6 Hz), 7.0-7.0 (m, 1H), 6.94 (d, 1H, J=1.0 Hz), 6.79 (dd, 1H, J=2.9, 10.3 Hz), 6.72 (s, 1H), 3.91 (s, 3H).

### Step C: Methyl 2-((2-(allyloxy)-4-fluorophenyl)amino)-4-(trifluoromethyl)benzoate 18c

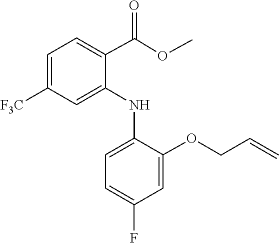 [SMILES: C=CCOc1cc(F)ccc1Nc1cc(C(F)(F)F)ccc1C(=O)OC]

Following the procedure outlined in Example 16, Step A, and stirring the reaction mixture at room temperature for 18 h, methyl 2-((2-(allyloxy)-4-fluorophenyl)amino)-4-(trifluoromethyl)benzoate 18c (1.68 g, 92% yield) was prepared as a yellow solid. HPLC/MS 1.47 min (A), [M+H]⁺ 370.0. ¹H NMR (DMSO-d₆, 400 MHz) δ 9.32 (s, 1H), 8.07 (d, 1H, J=8.3 Hz), 7.41 (dd, 1H, J=5.9, 8.8 Hz), 7.11 (s, 1H), 7.05 (d, 1H, J=8.3 Hz), 7.02 (s, 1H), 6.86 (d, 1H, J=2.4 Hz), 5.8-6.0 (m, 1H), 5.2-5.3 (m, 2H), 4.63 (td, 2H, J=1.7, 4.9 Hz), 3.91 (s, 3H).

### Step D: 2-((2-(Allyloxv)-4-fluorophenyl)amino)-4-(trifluoromethyl)benzoic acid 18d

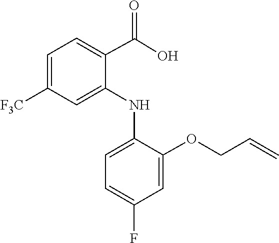 [SMILES: C=CCOc1cc(F)ccc1Nc1cc(C(F)(F)F)ccc1C(=O)O]

Following the procedure outlined in Example 16, Step B, and stirring the reaction mixture at 50° C. for 22 h, 2-((2-(allyloxy)-4-fluorophenyl)amino)-4-(trifluoromethyl)benzoic acid 18d (1.68 g, 92% yield) was prepared as a light yellow solid. HPLC/MS 1.33 min (A), [M+H]⁺ 356.0. ¹H NMR (DMSO-d₆, 400 MHz) δ 13.4-13.8 (m, 1H), 9.67 (s, 1H), 8.07 (d, 1H, J=7.8 Hz), 7.42 (dd, 1H, J=6.4, 8.8 Hz), 7.0-7.1 (m, 3H), 6.86 (dt, 1H, J=2.9, 8.6 Hz), 5.94 (dd, 1H, J=10.5, 17.4 Hz), 5.3-5.4 (m, 1H), 5.19 (qd, 1H, J=1.7, 10.6 Hz), 4.62 (td, 2H, J=1.5, 4.8 Hz).

### Step E: N-(2-Allyl-6-methoxypyridin-3-yl)-2-((2-(allyloxy)-4-fluorophenyl)amino)-4-(trifluoromethyl) benzamide 18e

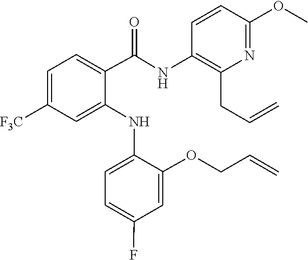 [SMILES: C=CCOc1cc(F)ccc1Nc1cc(C(F)(F)F)ccc1C(=O)Nc1ccc(OC)nc1CC=C]

To 2-((2-(allyloxy)-4-fluorophenyl)amino)-4-(trifluoromethyl)benzoic acid (894 mg, 2.52 mmol) and Int-1a (475 mg, 2.89 mmol), in MeCN (14 mL) and DMF (2.8 mL) was added TEA (3.51 mL, 25.2 mmol), followed by 2,4,6-tripropyl-1,3,5,2,4,6-trioxatriphosphinane-2,4,6-trioxide (3.20 g, 5.03 mmol) and the reaction mixture was stirred at room temperature for 18 h. The reaction mixture was diluted with EtOAc, washed with 1N NaOH, H₂O, brine, dried over Na₂SO₄, filtered and the solvent evaporated under reduced pressure. The crude residue was dissolved in DCM, absorbed onto a silica gel packed precolumn and purified by silica gel flash column chromatography (80 g) eluting with a 100% heptane to 25% EtOAc gradient. Pure product fractions were combined and evaporated under reduced pressure to afford N-(2-allyl-6-methoxypyridin-3-yl)-2-((2-(allyloxy)-4-fluorophenyl)amino)-4-(trifluoromethyl)benzamide 18e (931 mg, 72% yield) as a yellow solid. HPLC/MS 1.46 min (A), [M+H]⁺ 502.1. ¹H NMR (DMSO-d₆, 400 MHz) δ 10.21 (s, 1H), 9.38 (s, 1H), 8.02 (br d, 1H, J=8.3 Hz), 7.65 (d, 1H, J=8.8 Hz), 7.36 (dd, 1H, J=6.4, 8.8 Hz), 7.1-7.2 (m, 2H), 7.06 (dd, 1H, J=2.4, 10.8 Hz), 6.83 (dt, 1H, J=2.4, 8.6 Hz), 6.75 (d, 1H, J=8.3 Hz), 5.9-6.1 (m, 2H), 5.36 (dd, 1H, J=1.7, 17.4 Hz), 5.1-5.2 (m, 1H), 4.9-5.1 (m, 2H), 4.62 (td, 2H, J=1.5, 4.8 Hz), 3.87 (s, 3H), 3.5-3.6 (m, 2H).

### Step F: 3-(2-(Allyl-6-methoxypyridin-3-yl)-1-(2-(allyloxy)-4-fluorophenyl)-7-(trifluoro-methyl)-2,3-dihydro quinazolin-4-(1H)-one 18f

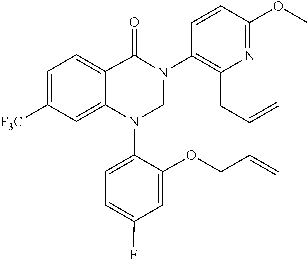 [SMILES: C=CCOc1cc(F)ccc1N1CN(c2ccc(OC)nc2CC=C)C(=O)c2ccc(C(F)(F)F)cc21]

Following the procedure outlined in Example 7, Step D, and stirring the reaction mixture at 65° C. for 1.5 h, 3-(2-(allyl-6-methoxypyridin-3-yl)-1-(2-(allyloxy)-4-fluorophenyl)-7-(trifluoromethyl)-2,3-dihydro quinazolin-4-(1H)-one 18f (776 mg, 79% yield) was prepared as a white solid. HPLC/MS 1.42 min (A), [M+H]⁺ 514.1. ¹H NMR (DMSO-d₆, 400 MHz) δ 8.07 (d, 1H, J=7.8 Hz), 7.70 (d, 1H, J=8.3 Hz), 7.40 (dd, 1H, J=6.1, 8.6 Hz), 7.24 (dd, 1H, J=1.2, 8.1 Hz), 7.15 (dd, 1H, J=2.7, 11.0 Hz), 6.91 (dt, 1H, J=2.7, 8.4 Hz), 6.76 (d, 1H, J=8.3 Hz), 6.58 (d, 1H, J=1.0 Hz), 5.8-6.0 (m, 2H), 5.46 (br d, 1H, J=10.3 Hz), 5.1-5.2 (m, 2H), 4.8-5.0 (m, 4H), 4.61 (td, 2H, J=1.5, 4.9 Hz), 3.86 (s, 3H), 3.3-3.4 (m, 2H).

### Step G: 3⁴-Fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclooctaphane-6-en-2⁴-one 182

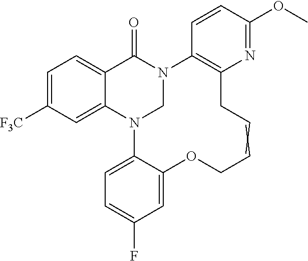 [SMILES: COc1ccc2c(n1)CC=CCOc1cc(F)ccc1N1CN2C(=O)c2ccc(C(F)(F)F)cc21]

Following the procedure outlined in Example 16, Step E, substituting Hoveyda-Grubbsii with Hoveyda-Grubbs M722 (Grubbs C₇₁₁), and stirring the reaction mixture at 80° C. for 135 h, 3⁴-fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclo octaphane-6-en-2⁴-one 18g (150 mg, 7% yield) was prepared as an off-white solid, and as a mixture of cis- and trans-isomers. HPLC/MS 1.29 (33%), 1.31 (66%) min (A), [M+H]⁺ 471.6.

### Step H: 3⁴-Fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclooctaphane-2⁴-one 18h

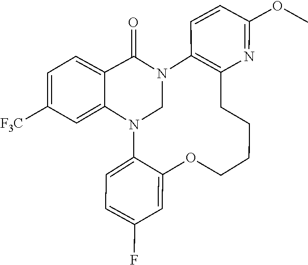 [SMILES: COc1ccc2c(n1)CCCCOc1cc(F)ccc1N1CN2C(=O)c2ccc(C(F)(F)F)cc21]

Following the procedure outlined in Example 10, Step G, stirring the reaction mixture with Pd(OH)₂ (20% wt on carbon) in EtOH/EtOAc (1:0.6) at room temperature for 1 h, 3⁴-fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclo octaphane-2⁴-one 18h (241 mg, 82% yield) was prepared as an off-white solid. HPLC/MS 1.33 min (C), [M+H]⁺ 488.1. ¹H-NMR (DMSO-d₆, 400 MHz) δ 8.05 (d, 1H, J=7.8 Hz), 7.68 (d, 1H, J=8.3 Hz), 7.58 (dd, 1H, J=6.4, 8.8 Hz), 7.1-7.2 (m, 2H), 6.92 (dt, 1H, J=2.7, 8.4 Hz), 6.78 (d, 1H, J=1.0 Hz), 6.73 (d, 1H, J=8.8 Hz), 5.46 (d, 1H, J=10.8 Hz), 4.64 (d, 1H, J=10.8 Hz), 4.2-4.3 (m, 1H), 4.0-4.1 (m, 1H), 3.86 (s, 3H), 3.2-3.3 (m, 1H), 2.4-2.4 (m, 1H), 2.4-2.5 (m, 1H), 1.9-2.1 (m, 1H), 1.5-1.9 (m, 3H).

### Step I: 3⁴-Fluoro-2⁷-(trifluoromethyl)-1¹ 1²,2¹,2²,2³,2⁴-hexahydro-4-oxa-2(3,1)-quinazolina-1(5,6)-pyridina-3(1,2)-benzenacyclooctaphane-1²,2⁴-dione Example 18

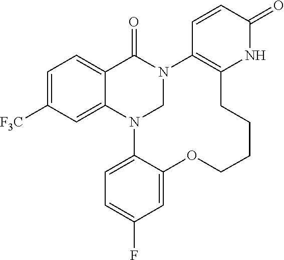 [SMILES: O=C1c2ccc(C(F)(F)F)cc2N2CN1c1ccc(=O)[nH]c1CCCCOc1cc(F)ccc12]

Following the procedure outlined in Example 16, Step G, and stirring the reaction mixture at 100° C. for 3 h, 3⁴-fluoro-2⁷-(trifluoromethyl)-1¹,1²,2¹,2²,2³,2⁴-hexahydro-4-oxa-2(3,1)-quinazolina-1(5,6)-pyridina-3(1,2)-benzenacyclooctaphane-1²,2⁴-dione (63 mg, 28% yield) was prepared as a white solid. HPLC/MS 1.09 min (A), [M+H]⁺ 474.1. ¹H-NMR (DMSO-d₆, 400 MHz) δ 11.72 (br s, 1H), 8.03 (d, 1H, J=7.8 Hz), 7.56 (dd, 1H, J=6.4, 8.8 Hz), 7.43 (d, 1H, J=9.3 Hz), 7.1-7.3 (m, 2H), 6.9-7.0 (m, 1H), 6.75 (s, 1H), 6.21 (br d, 1H, J=9.8 Hz), 5.40 (d, 1H, J=10.3 Hz), 4.61 (d, 1H, J=10.3 Hz), 4.2-4.4 (m, 1H), 4.00 (br d, 1H, J=3.9 Hz), 2.8-3.0 (m, 1H), 2.2-2.4 (m, 1H), 2.0-2.2 (m, 1H), 1.8-1.9 (m, 1H), 1.6-1.7 (m, 1H), 1.3-1.5 (m, 1H).

### Example 19

### 3⁴-Fluoro-2⁷-(trifluoromethyl)-1¹,1²,2¹,2²,2³,2⁴-hexahydro-4-oxa-2(3,1)-quinazolina-1(5,6)-pyridina-3(1,2)-benzenacyclonaphane-1²,2⁴-dione

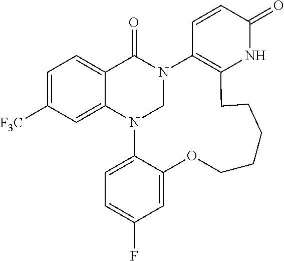 [SMILES: O=C1c2ccc(C(F)(F)F)cc2N2CN1c1ccc(=O)[nH]c1CCCCCOc1cc(F)ccc12]

### Step A: 2-((2-Allyloxy)-4-fluorophenyl)amino)-N-(2-but-3-en-1-yl)-6-methoxypyridin-3-yl)-4-(trifluoromethyl)benzamide 19a

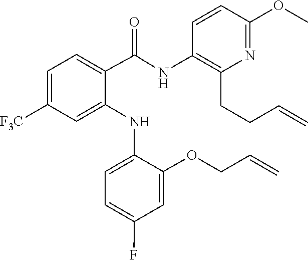 [SMILES: C=CCCc1nc(OC)ccc1NC(=O)c1ccc(C(F)(F)F)cc1Nc1ccc(F)cc1OCC=C]

Following the procedure outlined in Example 18, Step E, substituting Int-1a with Int-1b, and stirring the reaction mixture at room temperature in MeCN for 18 h, 2-((2-allyloxy)-4-fluorophenyl)amino)-N-(2-but-3-en-1-yl)-6-methoxypyridin-3-yl)-4-(trifluoromethyl)benzamide 19a (658 mg, 68% yield) was prepared as a light orange foam. HPLC/MS 1.50 min (A), [M+H]⁺ 516.2. ¹H-NMR (DMSO-d₆, 400 MHz) δ 10.1-10.3 (m, 1H), 9.2-9.5 (m, 1H), 8.0-8.1 (m, 1H), 7.62 (d, 1H, J=8.3 Hz), 7.3-7.4 (m, 1H), 7.1-7.2 (m, 2H), 7.05 (dd, 1H, J=2.4, 10.8 Hz), 6.8-6.9 (m, 1H), 6.72 (d, 1H, J=8.8 Hz), 5.8-6.0 (m, 2H), 5.38 (s, 1H), 5.18 (br d, 1H, J=10.8 Hz), 4.9-5.1 (m, 2H), 4.6-4.7 (m, 2H), 3.8-3.9 (m, 4H), 2.7-2.8 (m, 2H), 2.4-2.5 (m, 2H).

### Step B: 1-(2-(Allyloxy)-4-fluorophenyl)-3-(2-(but-3-en-1-yl)-6-methoxypyridin-3-yl)-7-(trifluoromethyl)-2,3-dihydroquinazolin-4(1H)-one 19b

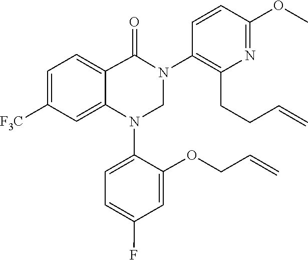 [SMILES: C=CCCc1nc(OC)ccc1N1CN(c2ccc(F)cc2OCC=C)c2cc(C(F)(F)F)ccc2C1=O]

Following the procedure outlined in Example 16, Step E, stirring the reaction mixture at 65° C. for 2 h, 1-(2-(allyloxy)-4-fluorophenyl)-3-(2-(but-3-en-1-yl)-6-methoxypyridin-3-yl)-7-(trifluoromethyl)-2,3-dihydro quinazolin-4(1H)-one 19b (238 mg, 35% yield) was prepared as a yellow oil. HPLC/MS 1.49 min (A), [M+H]⁺ 528.2. ¹H-NMR (DMSO-d₆, 400 MHz) δ 8.0-8.1 (m, 1H), 7.67 (d, 1H, J=8.3 Hz), 7.39 (br d, 1H, J=8.8 Hz), 7.2-7.3 (m, 1H), 7.1-7.2 (m, 2H), 6.9-7.0 (m, 1H), 6.7-6.8 (m, 1H), 6.59 (s, 1H), 5.72 (br d, 2H, J=6.8 Hz), 5.48 (br d, 2H, J=10.3 Hz), 5.12 (s, 3H), 4.7-4.9 (m, 4H), 4.70 (s, 1H), 4.6-4.6 (m, 3H), 3.86 (s, 3H), 3.85 (s, 1H), 2.6-2.7 (m, 2H), 2.3-2.4 (m, 2H).

### Step C: 3⁴-Fluoro-1⁶-methoxy-2⁷-(trifluoromethyl)-2¹,2²,2³,2⁴-tetrahydro-4-oxa-2(3,1)-quinazolina-1(3,2)-pyridina-1(3,2)-benzenacyclonaphan-7-en-2⁴-one 19c

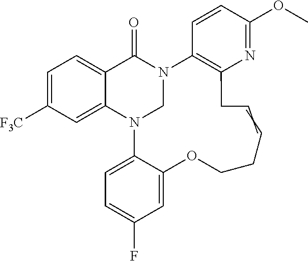 [SMILES: COc1ccc2c(n1)CC=CCCOc1cc(F)ccc1N1CN2C(=O)c2ccc(C(F)(F)F)cc21]

Following the procedure outlined in Example 16, Step E, stirring the rection mixture at 65° C. for 2 h, 3⁴-fluoro-1⁶-methoxy-2⁷-(trifluoromethyl)-2¹,2²,2³,2⁴-tetrahydro-4-oxa-2(3,1)-quinazolina-1(3,2)-pyridina-1(3,2)-benzenacyclonaphan-7-en-2⁴-one 19c (90 mg, 41% yield) was prepared as a white solid, and as a mixture of cis- and trans-isomers. HPLC/MS 1.34, 1.38 min (A), [M+H]⁺ 500.1.

### Step D: 3⁴-Fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclonaphan-2⁴-one 19d

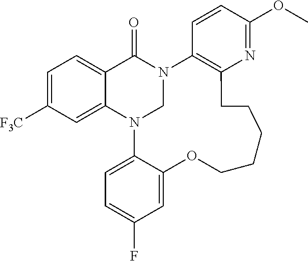 [SMILES: COc1ccc2c(n1)CCCCCOc1cc(F)ccc1N1CN2C(=O)c2ccc(C(F)(F)F)cc21]

Following the procedure outlined in Example 10, Step G, stirring the reaction mixture with Pd(OH)₂ (20% wt on carbon) in EtOH/EtOAc (1:0.6) at room temperature for 18 h, 3⁴-fluoro-1⁶-methoxy-2⁷-(trifluoromethyl)-2¹,2²,2³,2⁴-tetrahydro-4-oxa-2(3,1)-quinazolina-1(3,2)-pyridina-3(1,2)-benzenacyclonaphan-2⁴-one 19d (89 mg, 100% yield), in 78% purity, was prepared as a white solid. HPLC/MS 1.39 min (A), [M+H]⁺ 502.1.

### Step E: 3⁴-Fluoro-2⁷-(trifluoromethyl)-1¹ 1²,2¹,2²,2³,2⁴-hexahydro-4-oxa-2(3,1)-quinazolina-1(5,6)-pyridina-3(1,2)-benzenacyclonaphane-1²,2⁴-dione Example 19

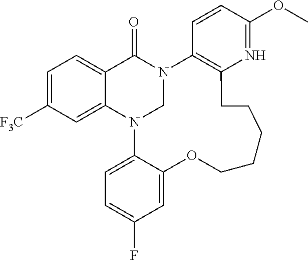 [SMILES: COc1ccc2c(n1)CCCCCOc1cc(F)ccc1N1CN2C(=O)c2ccc(C(F)(F)F)cc21]

Following the procedure outlined in Example 15, Step G, stirring the reaction mixture at 100° C. for 5 h, 3⁴-fluoro-2⁷-(trifluoromethyl)-1¹,1²2¹,2²,2³,2⁴-hexahydro-4-oxa-2(3,1)-quinazolina-1(5,6)-pyridina-3(1,2)-benzenacyclonaphane-1²,2⁴-dione (35 mg, 40% yield) was prepared as a white solid. HPLC/MS 1.09 min (A), [M+H]⁺ 488.1. ¹H-NMR (DMSO-d₆, 400 MHz) δ 11.4-12.1 (m, 1H), 8.03 (d, 1H, J=8.3 Hz), 7.51 (dd, 1H, J=6.4, 8.8 Hz), 7.42 (d, 1H, J=9.8 Hz), 7.1-7.2 (m, 2H), 6.93 (dt, 1H, J=2.9, 8.6 Hz), 6.55 (d, 1H, J=1.0 Hz), 6.22 (d, 1H, J=9.3 Hz), 5.49 (d, 1H, J=9.8 Hz), 4.49 (d, 1H, J=9.8 Hz), 4.1-4.2 (m, 1H), 4.02 (br dd, 1H, J=4.4, 10.8 Hz), 2.8-2.9 (m, 1H), 2.1-2.3 (m, 1H), 1.76 (br d, 3H, J=5.4 Hz), 1.5-1.7 (m, 3H).

### Example 20

### 2⁶-Chloro-3⁴-fluoro-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione

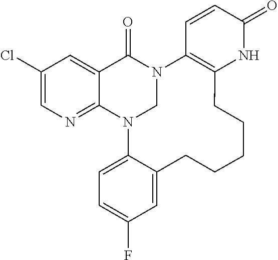 [SMILES: O=C1c2cc(Cl)cnc2N2CN1c1ccc(=O)[nH]c1CCCCCc1cc(F)ccc12]

### Step A: Ethyl 2-((2-allyl-4-fluorophenyl)amino)-5-chloronicotinate 20a

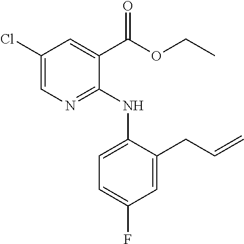 [SMILES: C=CCc1cc(F)ccc1Nc1ncc(Cl)cc1C(=O)OCC]

Following the procedure outlined in Example 7, Step A, substituting ethyl 2-bromo-5-(trifluoromethyl)benzoate with ethyl 2-bromo-5-chloronicotinate, Pd(OAc)₂ with Pd₂(dba)₃ and stirring the reaction mixture in 1,4-dioxane at 100° C. for 4 h, ethyl 2-((2-allyl-4-fluorophenyl)amino)-5-chloronicotinate 20a (2.71 g, 76% yield) was prepared as a yellow solid. HPLC/MS 1.53 min (B), [M+H]⁺ 335.1. ¹H NMR (CDCl₃, 400 MHz) δ 9.68 (br s, 1H), 8.23 (br d, J=7.3 Hz, 2H), 7.69 (br dd, J=7.3, 5.9 Hz, 1H), 6.94-7.07 (m, 2H), 5.98 (td, J=10.3, 6.4 Hz, 1H), 5.02-5.22 (m, 2H), 4.43 (q, J=7.2 Hz, 2H), 3.41 (br d, J=5.9 Hz, 2H), 1.45 (br t, J=6.9 Hz, 3H).

### Step B: 2-((2-Allyl-4-fluorophenyl)amino)-5-chloronicotinic acid 20b

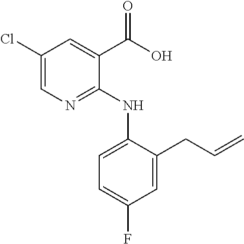 [SMILES: C=CCc1cc(F)ccc1Nc1ncc(Cl)cc1C(=O)O]

Following the procedure outlined in Example 7, Step B, stirring the reaction mixture in a THF/MeOH/H₂O solvent system at room temperature for 2 h, 2-((2-allyl-4-fluorophenyl)amino)-5-chloronicotinic acid 20b (2.30 g, 93% yield) was prepared as a white solid. HPLC/MS 0.76 min (B), [M+H]⁺ 307.0. ¹H NMR (CD₃OD, 400 MHz) δ 8.22 (d, J=2.9 Hz, 1H), 8.15 (d, J=2.5 Hz, 1H), 7.75 (dd, J=8.8, 5.4 Hz, 1H), 6.91-7.01 (m, 2H), 5.85-6.01 (m, 1H), 4.95-5.09 (m, 2H), 3.33-3.40 (m, 2H).

### Step C: 2-((2-Allyl-4-fluorophenyl)amino)-N-(2-(but-3-en-1-yl)-6-methoxypyridin-3-yl)-5-chloronicotinamide 20c

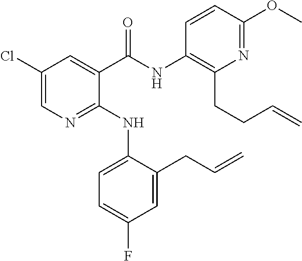 [SMILES: C=CCCc1nc(OC)ccc1NC(=O)c1cc(Cl)cnc1Nc1ccc(F)cc1CC=C]

Following the procedure outlined in Example 7, Step C, substituting Int-1a with Int-1b, stirring the reaction mixture in MeCN at room temperature for 2 h, 2-((2-allyl-4-fluorophenyl)amino)-N-(2-(but-3-en-1-yl)-6-methoxypyridin-3-yl)-5-chloronicotinamide 20c (763 mg, 47% yield) was prepared as a yellow solid. HPLC/MS 1.57 min (B), [M+H]⁺ 467.1. ¹H NMR (CDCl₃, 400 MHz) δ 9.75 (s, 1H), 8.20 (d, J=2.5 Hz, 1H), 7.79 (d, J=2.5 Hz, 1H), 7.59-7.72 (m, 3H), 6.88-6.98 (m, 2H), 6.64 (d, J=8.8 Hz, 1H), 5.91 (ddt, J=13.5, 6.8, 3.3, 3.3 Hz, 2H), 4.98-5.15 (m, 4H), 3.92-3.97 (m, 3H), 3.35 (br d, J=6.4 Hz, 2H), 2.71-2.84 (m, 2H), 2.56 (q, J=7.0 Hz, 2H).

### Step D: 1-(2-Allyl-4-fluorophenyl)-3-(2-(but-3-en-1-yl)-6-methoxypyridin-3-yl)-6-chloro-2,3-dihydropyrido[2,3-d]pyrimidin-4(11H)-one 20d

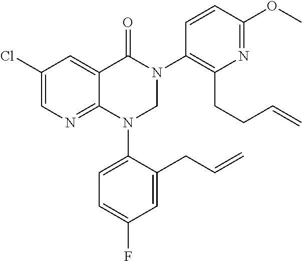 [SMILES: C=CCCc1nc(OC)ccc1N1CN(c2ccc(F)cc2CC=C)c2ncc(Cl)cc2C1=O]

Following the procedure outlined in Example 11, Step E, stirring the reaction mixture in MeCN at 80° C. for 23.5 h, then adding CH₂I₂ (3 equiv) and stirring at 80° C. for an additional 2 h, 1-(2-allyl-4-fluorophenyl)-3-(2-(but-3-en-1-yl)-6-methoxypyridin-3-yl)-6-chloro-2,3-dihydropyrido[2,3-d]pyrimidin-4(1H)-one 20d (170 mg, 35% yield) was prepared as a yellow oil. HPLC/MS 1.45 min (B), [M+H]⁺ 479.3. ¹H NMR (CDCl₃, 400 MHz) δ 8.26-8.30 (m, 1H), 8.20 (d, J=2.9 Hz, 1H), 7.41 (br d, J=7.8 Hz, 1H), 6.93-7.22 (m, 3H), 6.64 (d, J=8.7 Hz, 1H), 5.75-5.98 (m, 2H), 4.63-5.48 (m, 6H), 3.89-3.94 (m, 3H), 3.27-3.44 (m, 2H), 2.66-2.89 (m, 2H), 2.48-2.58 (m, 2H).

### Step E: 2⁶-Chloro-3⁴-fluoro-1⁶-methoxy-2¹,2²,2³,2⁴-tetrahydro-2(3,1)-pyrido[2,3-d]pyrimidina-1(3,2)-pyridina-3(1,2)-benzenacyclooctaphan-5-en-2⁴-one 20e

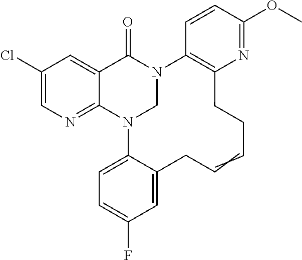 [SMILES: COc1ccc2c(n1)CCC=CCc1cc(F)ccc1N1CN2C(=O)c2cc(Cl)cnc21]

Following the procedure outlined in Example 7, Step E, stirring the reaction mixture at 80° C. for 1 h, 2⁶-chloro-3⁴-fluoro-1⁶-methoxy-2¹,2²,2³,2⁴-tetrahydro-2(3,1)-pyrido[2,3-d]pyrimidina-1(3,2)-pyridina-3(1,2)-benzenacyclooctaphan-5-en-2⁴-one 20e (117 mg, 51% yield) was prepared as a yellow oil, and as a mixture of cis- and trans-isomers. HPLC/MS 1.32 min (B), [M+H]⁺ 450.1. ¹H NMR (CDCl₃, 400 MHz) δ 8.14-8.20 (m, 1H), 8.04-8.10 (m, 1H), 7.17-7.28 (m, 1H), 6.97-7.06 (m, 1H) 6.83-6.95 (m, 2H), 6.50 (t, J=8.7 Hz, 1H), 5.20-5.59 (m, 3H), 4.30-4.57 (m, 1H), 3.77-3.83 (m, 3H), 3.24 (dd, J=14.9, 9.5 Hz, 1H), 2.92-3.09 (m, 1H), 2.60-2.85 (m, 2H), 2.38-2.56 (m, 1H), 2.27 (br d, J=17.6 Hz, 1H).

### Step F: 2⁶-Chloro-3⁴-fluoro-1⁶-methoxy-2¹,2²,2³,2⁴-tetrahydro-2(3,1)-pyrido[2,3-d]pyrimidina-1(3,2)-pyridina-3(1,2)-benzenacyclooctaphan-2⁴-one 20f

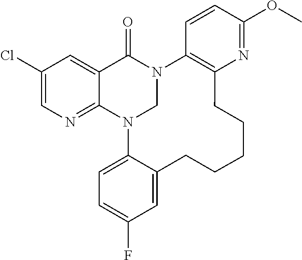 [SMILES: COc1ccc2c(n1)CCCCCc1cc(F)ccc1N1CN2C(=O)c2cc(Cl)cnc21]

To a solution of 2⁶-chloro-3⁴-fluoro-1⁶-methoxy-2¹,2²,2³,2⁴-tetrahydro-2(3,1)-pyrido[2,3-d]pyrimidina-1(3,2)-pyridina-3(1,2)-benzenacyclooctaphan-5-en-2⁴-one (213 mg, 0.472 mmol), dissolved in DCE (5 mL) and MeOH (0.250 mL) was added Grubbs II catalyst M204 (40.1 mg, 0.047 mmol) followed by NaBH₄ (35.7 mg, 0.945 mmol) and reaction was stirred at room temperature for 16 h. The reaction mixture was quenched with H₂O, extracted with EtOAc, the layers separated, and the aqueous layer extracted with EtOAc. The combined organic extracts were washed with brine, dried over MgSO₄, filtered and the solvent evaporated under reduced pressure. The residue was dissolved in DCM, adsorbed on Biotage Isolute HN—N, evaporated to dryness, and purified by silica gel flash column chromatography (24 g), eluting with a 100% heptanes to 100% ethyl acetate gradient. Product fractions were combined and evaporated under reduced pressure to afford 2⁶-chloro-3⁴-fluoro-1⁶-methoxy-2¹,2²,2³,2⁴-tetrahydro-2(3,1)-pyrido[2,3-d]pyrimidina-1(3,2)-pyridina-3(1,2)-benzenacyclooctaphan-2⁴-one 20f (87 mg, 39% yield) as a white powder. HPLC/MS 1.34 min (B), [M+H]⁺ 453.1. ¹H NMR (CDCl₃, 400 MHz) δ 8.21 (d, J=2.5 Hz, 1H), 8.11 (d, J=2.5 Hz, 1H), 7.27 (d, J=8.3 Hz, 1H), 6.99 (dd, J=8.6, 5.1 Hz, 1H), 6.90 (dd, J=9.3, 2.9 Hz, 1H), 6.82-6.88 (m, 1H), 6.51 (d, J=8.8 Hz, 1H), 5.57 (d, J=10.8 Hz, 1H), 4.57 (d, J=10.8 Hz, 1H), 3.79 (s, 3H), 2.57-2.74 (m, 2H), 2.50 (dt, J=14.3, 7.3 Hz, 1H), 2.32 (dt, J=14.7, 7.3 Hz, 1H), 1.62-1.81 (m, 2H), 1.47-1.57 (m, 1H), 1.41 (dt, J=13.6, 6.7 Hz, 1H), 1.17-1.27 (m, 2H).

### Step G: 2⁶-Chloro-3⁴-fluoro-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione Example 20

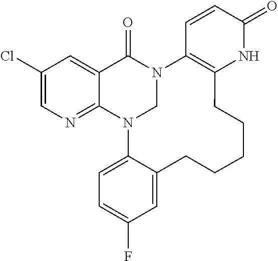 [SMILES: O=C1c2cc(Cl)cnc2N2CN1c1ccc(=O)[nH]c1CCCCCc1cc(F)ccc12]

Following the procedure outlined in Example 7, Step G, stirring the reaction mixture at 100° C. for 40 h, then adding additional pTsOH (3 equiv) and LiCl (3 equiv) and continued heating at 100° C. for 3 h, 2⁶-chloro-3⁴-fluoro-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione (50 mg, 59% yield) was prepared as a white solid. HPLC/MS 0.92 min (B), [M+H]⁺ 439.1. ¹H NMR (DMSO-d₆, 400 MHz) δ 11.68 (br s, 1H), 8.24-8.39 (m, 1H) 8.11-8.23 (m, 1H), 7.40 (d, J=9.8 Hz, 1H), 7.28 (dd, J=8.6, 5.6 Hz, 1H), 7.06-7.22 (m, 2H), 6.22 (br d, J=9.3 Hz, 1H), 5.77 (d, J=11.3 Hz, 1H), 4.79 (d, J=10.7 Hz, 1H), 2.25-2.47 (m, 4H), 1.71-1.84 (m, 1H), 1.58-1.71 (m, 1H), 1.41 (td, J=13.7, 6.9 Hz, 2H), 1.06-1.23 (m, 2H).

### Example 21

### 3⁴-Fluoro-2⁶-(trifluoromethyl)-1¹,1²,2¹,2²,2³,2⁴-hexahydro-2(3,1)-pyrido[3,4-d]pyrimidina-1(5,6)-pyridina-3(1,2)-benzenacyclooctaphane-1²,2⁴-dione

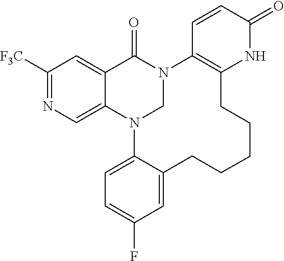 [SMILES: O=C1c2cc(C(F)(F)F)ncc2N2CN1c1ccc(=O)[nH]c1CCCCCc1cc(F)ccc12]

### Step A: Methyl 5-((2-allyl-4-fluorophenyl)amino)-2-(trifluoromethyl)isonicotinate 21a

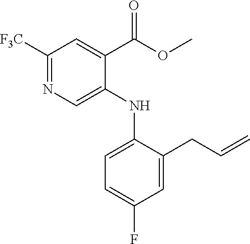 [SMILES: C=CCc1cc(F)ccc1Nc1cnc(C(F)(F)F)cc1C(=O)OC]

Following the procedure outlined in Example 7, Step A, substituting Int-3a with Int-3b, Pd(OAc)₂ with Pd₂(dba)₃, toluene with 1,4-dioxane, and stirring the reaction mixture at 100° C. for 4 h, methyl 5-((2-allyl-4-fluorophenyl)amino)-2-(trifluoromethyl)isonicotinate 21a (2.91 g, 77% yield) was prepared as a yellow solid. HPLC/MS 1.47 min (B), [M+H]⁺ 355.0. ¹H NMR (CDCl₃, 400 MHz) δ 10.22 (s, 1H), 8.61 (d, J=7.3 Hz, 1H), 8.31 (dd, J=8.8, 5.4 Hz, 1H), 7.19-7.28 (m, 3H), 6.27 (dd, J=17.1, 10.3 Hz, 1H), 5.29-5.43 (m, 2H), 4.19-4.24 (m, 3H), 3.67-3.71 (m, 2H).

### Step B: 5-((2-Allyl-4-fluorophenyl)amino)-2-(trifluoromethyl)isonicotinic acid 21b

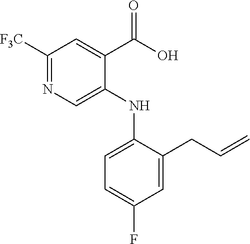 [SMILES: C=CCc1cc(F)ccc1Nc1cnc(C(F)(F)F)cc1C(=O)O]

Following the procedure outlined in Example 7, Step B, stirring the reaction mixture in a THF/MeOH/H₂O solvent system at room temperature for 2 h, 5-((2-allyl-4-fluorophenyl)amino)-2-(trifluoromethyl) isonicotinic acid 21b (2.45 g, 89% yield) was prepared as a yellow solid. HPLC/MS 0.88 min (B), [M+H]⁺ 341.0. ¹H NMR (CD₃OD, 400 MHz) δ 8.49 (d, J=7.8 Hz, 1H), 8.01 (t, J=6.4 Hz, 1H), 7.12 (d, J=7.8 Hz, 1H), 6.94-7.02 (m, 2H), 5.92-6.05 (m, 1H), 5.00-5.10 (m, 2H), 3.40-3.47 (m, 2H).

### Step C: 5-((2-Allyl-4-fluorophenyl)amino)-N-(2-(but-3-en-1-yl)-6-methoxypyridin-3-yl)-2-(trifluoromethyl) isonicotinamide 21c

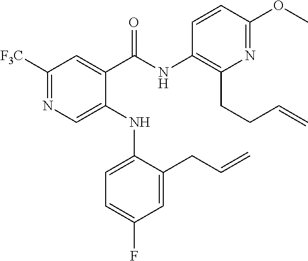 [SMILES: C=CCCc1nc(OC)ccc1NC(=O)c1cc(C(F)(F)F)ncc1Nc1ccc(F)cc1CC=C]

Following the procedure outlined in Example 7, Step C, substituting Int-1a with Int-1b, stirring the reaction mixture at room temperature for 2 h, 5-((2-allyl-4-fluorophenyl)amino)-N-(2-(but-3-en-1-yl)-6-methoxypyridin-3-yl)-2-(trifluoromethyl)isonicotinamide 21c (1.02 g, 57% yield) was prepared as a yellow solid. HPLC/MS 1.55 min (B), [M+H]⁺ 501.1. ¹H NMR (CDCl₃, 400 MHz) δ 9.33 (s, 1H), 8.17 (s, 1H), 8.29 (s, 1H), 7.76 (s, 1H), 7.64 (d, J=8.8 Hz, 1H), 7.24 (dd, J=8.6, 5.1 Hz, 1H), 7.06 (dd, J=9.1, 2.7 Hz, 1H), 6.97-7.03 (m, 1H), 6.63 (d, J=8.3 Hz, 1H), 5.82-5.96 (m, 2H), 4.97-5.14 (m, 4H), 3.94-3.97 (m, 3H), 3.34 (br d, J=6.4 Hz, 2H), 2.80 (t, J=7.6 Hz, 2H), 2.54 (q, J=7.0 Hz, 2H).

### Step D: 1-(2-Allyl-4-fluorophenyl)-3-(2-(but-3-en-1-yl)-6-methoxypyridin-3-yl)-6-(trifluoromethyl)-2,3-dihydropyrido[3,4-d]pyrimidin-4(11H)-one 21d

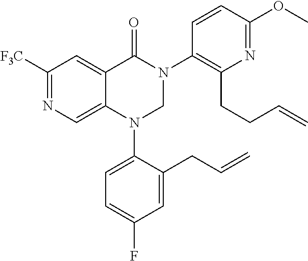 [SMILES: C=CCCc1nc(OC)ccc1N1CN(c2ccc(F)cc2CC=C)c2cnc(C(F)(F)F)cc2C1=O]

Following the procedure outlined in Example 11, Step E, stirring the reaction mixture at 80° C. for 17.5 h, then adding additional CH₂I₂ (3 equiv) and stirring continued at 80° C. for 23.5 h, 1-(2-allyl-4-fluorophenyl)-3-(2-(but-3-en-1-yl)-6-methoxypyridin-3-yl)-6-(trifluoromethyl)-2,3-dihydropyrido[3,4-d]pyrimidin-4(1H)-one 21d (131 mg, 12% yield) was prepared as a yellow oil. HPLC/MS 1.49 min (B), [M+H]⁺ 513.1. ¹H NMR (CDCl₃, 400 MHz) δ 8.23-8.28 (m, 1H), 7.76-7.87 (m, 1H), 7.41 (br t, J=8.1 Hz, 1H), 7.04-7.28 (m, 3H), 6.62-6.70 (m, 1H), 5.78-6.01 (m, 2H), 4.94-5.13 (m, 4H), 3.92-4.01 (m, 3H), 3.34-3.48 (m, 2H), 2.64-2.92 (m, 2H), 2.51-2.61 (m, 2H).

### Step E: 3⁴-Fluoro-1⁶-methoxy-2⁶-(trifluoromethyl)-2¹,2²,2³,2⁴-tetrahydro-2(3,1)-pyrido-[3,4-d]pyrimidina-1(3,2)-pyridina-3(1,2)-benzenacyclooctaphan-5-en-2⁴-one 21e

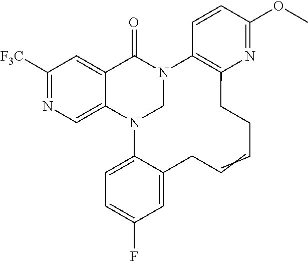 [SMILES: COc1ccc2c(n1)CCC=CCc1cc(F)ccc1N1CN2C(=O)c2cc(C(F)(F)F)ncc21]

Following the procedure outlined in Example 7, Step E, stirring the reaction mixture at 80° C. for 1 h, 3⁴-fluoro-1⁶-methoxy-2⁶-(trifluoromethyl)-2¹,2²,2³,2⁴-tetrahydro-2(3,1)-pyrido[3,4-d]pyrimidina-1(3,2)-pyridina-3(1,2)-benzenacyclooctaphan-5-en-2⁴-one 21e (37 mg, 32% yield) was prepared as a clear oil, and as a mixture of cis- and trans-isomers. HPLC/MS 1.39 min (B), [M+H]⁺ 485.2.

### Step F: 3⁴-Fluoro-1⁶-methoxy-2⁶-(trifluoromethyl)-2¹,2²,2³,2⁴-tetrahydro-2(3,1)-pyrido-[3,4-d]pyrimidina-1(3,2)-pyridina-3(1,2)-benzenacyclooctaphan-2⁴-one 21f

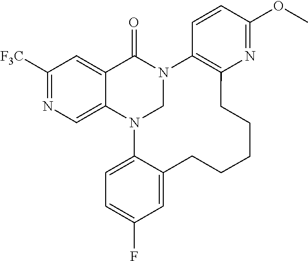 [SMILES: COc1ccc2c(n1)CCCCCc1cc(F)ccc1N1CN2C(=O)c2cc(C(F)(F)F)ncc21]

Following the procedure outlined in Example 7, Step F, replacing MeOH with EtOH/EtOAc (1:0.4) and stirring for 17 h, 3⁴-fluoro-1⁶-methoxy-2⁶-(trifluoromethyl)-2¹,2²,2³,2⁴-tetrahydro-2(3,1)-pyrido[3,4-d]pyrimidina-1(3,2)-pyridina-3(1,2)-benzenacyclooctaphan-2⁴-one 21f (37 mg, 34% yield) was prepared as a white solid. HPLC/MS 1.38 min (A), [M+H]⁺ 487.2. ¹H NMR (CDCl₃, 400 MHz) δ 8.15 (s, 1H), 7.72 (s, 1H), 7.27 (d, J=8.8 Hz, 1H), 7.00-7.08 (m, 1H), 6.87-6.98 (m, 2H), 6.51 (d, J=8.8 Hz, 1H), 5.54 (d, J=10.8 Hz, 1H), 4.55 (d, J=10.3 Hz, 1H), 3.73-3.82 (m, 3H), 2.65 (tt, J=14.4, 7.2 Hz, 2H) 2.43-2.56 (m, 1H), 2.29-2.43 (m, 1H), 1.63-1.80 (m, 2H), 1.49-1.57 (m, 1H), 1.35-1.46 (m, 1H), 1.12-1.30 (m, 2H).

### Step G: 3⁴-Fluoro-2⁶-(trifluoromethyl)-1¹1²,2¹,2²,2³,2⁴-hexahydro-2(3,1)-pyrido[3,4-d]pyrimidina-1(5,6)-pyridina-3(1,2)-benzenacyclooctaphane-1²,2⁴-dione

### Example 21

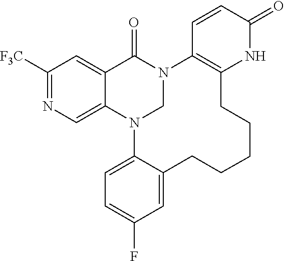 [SMILES: O=C1c2cc(C(F)(F)F)ncc2N2CN1c1ccc(=O)[nH]c1CCCCCc1cc(F)ccc12]

Following the procedure outlined in Example 7, and using Step G, stirring the reaction mixture at 100° C. for 2 h, 3⁴-fluoro-2⁶-(trifluoromethyl)-1¹,1²,2¹,2²,2³,2⁴-hexahydro-2(3,1)-pyrido[3,4-d]pyrimidina-1(5,6)-pyridina-3(1,2)-benzenacyclo octaphane-1²,2⁴-dione (23 mg, 49% yield) was prepared as a white solid. HPLC/MS 0.98 min (A), [M+H]⁺ 473.2. ¹H NMR (CD₃OD, 400 MHz) δ 8.19 (s, 1H), 7.83 (s, 1H), 7.48-7.64 (m, 1H), 7.40 (dd, J=8.8, 5.4 Hz, 1H), 7.06-7.28 (m, 2H), 6.44 (d, J=9.3 Hz, 1H), 5.79 (d, J=10.8 Hz, 1H), 4.87-4.96 (m, 1H), 2.40-2.75 (m, 4H), 1.86-2.04 (m, 1H), 1.71-1.86 (m, 1H), 1.45-1.68 (m, 2H), 1.25-1.45 (m, 2H).

### Example 22

### 3⁴-Fluoro-2⁷-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione

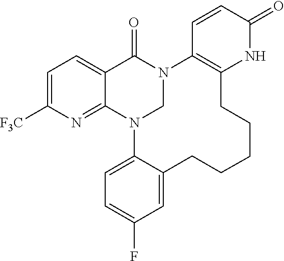 [SMILES: O=C1c2ccc(C(F)(F)F)nc2N2CN1c1ccc(=O)[nH]c1CCCCCc1cc(F)ccc12]

### Step A: Methyl 2-((2-allyl-4-fluorophenyl)amino)-6-(trifluoromethyl)nicotinate 22a

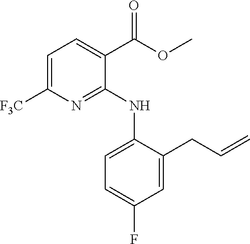 [SMILES: C=CCc1cc(F)ccc1Nc1nc(C(F)(F)F)ccc1C(=O)OC]

Following the procedure outlined in Example 7, Step A, substituting Int-3a with Int-3b, Pd(OAc)₂ with Pd₂(dba)₃, toluene with 1,4-dioxane, and stirring the reaction mixture at 100° C. for 4 h, methyl 5-((2-allyl-4-fluorophenyl)amino)-2-(trifluoromethyl)isonicotinate 22a (2.81 g, 75% yield) was prepared as a yellow oil. HPLC/MS 1.41 min (B), [M+H]⁺ 355.0 ¹H NMR (CDCl₃, 400 MHz) δ 9.18 (s, 1H), 8.19 (s, 1H), 8.08 (s, 1H), 7.28 (dd, J=8.6, 5.1 Hz, 1H), 7.07 (dd, J=9.3, 2.9 Hz, 1H), 7.00 (td, J=8.1, 2.9 Hz, 1H), 5.91 (dd, J=17.1, 10.3 Hz, 1H), 5.11-5.15 (m, 1H), 4.95-5.04 (m, 1H), 3.99 (s, 3H), 3.36 (d, J=6.36 Hz, 2H).

### Step B: 2-((2-Allyl-4-fluorophenyl)amino)-6-(trifluoromethyl)nicotinic acid 22b

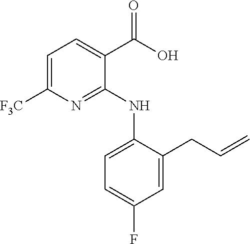 [SMILES: C=CCc1cc(F)ccc1Nc1nc(C(F)(F)F)ccc1C(=O)O]

Following the procedure outlined in Example 7, Step B, stirring the reaction mixture in a THF/MeOH/H₂O solvent system at room temperature for 2 h, 2-((2-allyl-4-fluorophenyl)amino)-2-(trifluoromethyl) isonicotinic acid 22b (2.41 g, 87% yield) was prepared as a yellow solid. HPLC/MS 1.58 min (B), [M+H]⁺ 475.1. ¹H NMR (CDCl₃, 400 MHz) δ 8.11 (s, 1H), 8.07 (s, 1H), 7.40 (dd, J=8.56, 5.14 Hz, 1H), 7.17 (dd, J=9.29, 2.93 Hz, 1H), 7.09-7.14 (m, 1H), 5.93 (dd, J=17.12, 10.27 Hz, 1H), 4.90-5.08 (m, 2H), 3.35-3.42 (m, 2H).

### Step C: 2-((2-Allyl-4-fluorophenyl)amino)-N-(2-(but-3-en-1-yl)-6-methoxypyridin-3-yl)-6-(trifluoromethyl) nicotinamide 22c

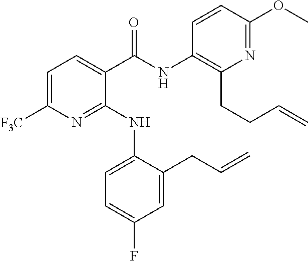 [SMILES: C=CCCc1nc(OC)ccc1NC(=O)c1ccc(C(F)(F)F)nc1Nc1ccc(F)cc1CC=C]

Following the procedure outlined in Example 7, Step C, substituting Int-1a with Int-1b, and stirring the reaction mixture in MeCN at room temperature for 2 h, 2-((2-allyl-4-fluorophenyl)amino)-N-(2-(but-3-en-1-yl)-6-methoxypyridin-3-yl)-6-(trifluoromethyl)nicotinamide 22c (751 mg, 45% yield) was prepared as a yellow solid. HPLC/MS 1.59 min (B), [M+H]⁺ 501.1. ¹H NMR (CDCl₃, 400 MHz) δ 10.26 (br s, 1H), 8.21 (br dd, J=8.6, 5.6 Hz, 1H), 8.15 (br d, J=7.8 Hz, 1H), 7.94 (br d, J=8.3 Hz, 1H), 7.84 (br s, 1H), 7.27 (br d, J=7.3 Hz, 1H), 7.10-7.21 (m, 2H), 6.88 (d, J=8.8 Hz, 1H), 6.02-6.23 (m, 2H), 5.18-5.37 (m, 4H), 4.12-4.23 (m, 3H), 3.61 (br d, J=5.9 Hz, 3H), 2.94-3.10 (m, 2H), 2.71-2.85 (m, 2H).

### Step D: 1-(2-Allyl-4-fluorophenyl)-3-(2-(but-3-en-1-yl)-6-methoxypyridin-3-yl)-7-(trifluoromethyl)-2,3-dihydropyrido[2,3-d]pyrimidin-4(11H)-one 22d

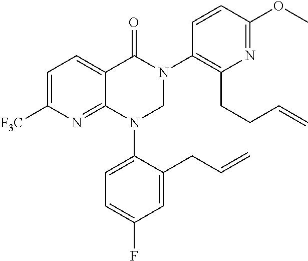 [SMILES: C=CCCc1nc(OC)ccc1N1CN(c2ccc(F)cc2CC=C)c2nc(C(F)(F)F)ccc2C1=O]

Following the procedure outlined in Example 11, Step E, stirring the reaction mixture at 80° C. for 17.5 h, then adding additional CH₂I₂ (6 equiv) and stirring continued at 80° C. for 23.5 h, 1-(2-allyl-4-fluorophenyl)-3-(2-(but-3-en-1-yl)-6-methoxypyridin-3-yl)-7-(trifluoromethyl)-2,3-dihydropyrido[2,3-d]pyrimidin-4(1H)-one 22d (409 mg, 57% yield) was prepared as a yellow oil. HPLC/MS 1.53 min (B), [M+H]⁺ 513.2. ¹H NMR (CDCl₃, 400 MHz) δ 8.48 (d, J=7.83 Hz, 1H) 7.43-7.50 (m, 1H) 7.25 (d, J=7.83 Hz, 1H), 6.97-7.21 (m, 3H), 6.69 (d, J=8.31 Hz, 1H), 5.80-5.99 (m, 2H), 4.75-5.29 (m, 6H), 3.95-4.01 (m, 3H), 3.37 (br dd, J=15.41, 5.62 Hz, 2H), 2.70-2.94 (m, 2H), 2.54-2.66 (m, 2H).

### Step E: 3⁴-Fluoro-1⁶-methoxy-2⁷-(trifluoromethyl)-2¹,2²,2³,2⁴-tetrahydro-2(3.1)-pyrido-[2,3-d]pyrimidina-1(3,2)-pyridina-3(1,2)-benzenacyclooctaphan-5-en-2⁴-one 22e

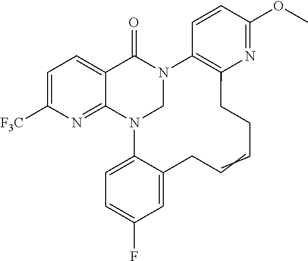 [SMILES: COc1ccc2c(n1)CCC=CCc1cc(F)ccc1N1CN2C(=O)c2ccc(C(F)(F)F)nc21]

Following the procedure outlined in Example 7, Step E, stirring the reaction mixture in DCE at 80° C. for 1 h, 3⁴-fluoro-1⁶-methoxy-2⁷-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-5-en-2⁴-one 22e (311 mg, 76% yield) was prepared as an oily yellow solid, and as a mixture of cis- and trans-isomers. HPLC/MS 1.39 min (B), [M+H]⁺ 485.2. ¹H NMR (CDCl₃, 400 MHz) δ 8.41-8.53 (m, 1H), 7.31-7.41 (m, 1H), 6.96-7.29 (m, 4H), 6.59-6.69 (m, 1H), 5.36-5.78 (m, 3H), 4.48-4.76 (m, 1H), 3.90-3.99 (m, 3H).

### Step F: 3⁴-Fluoro-1⁶-methoxy-2⁷-(trifluoromethyl)-2¹,2²,2³,2⁴-tetrahydro-2(3,1)-pyrido-[2,3-d]pyrimidina-1(3,2)-pyridina-3(1,2)-benzenacyclooctaphan-2⁴-one 22f

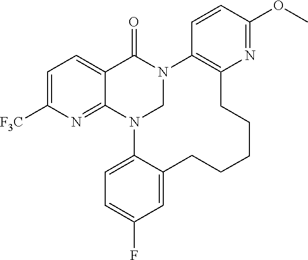 [SMILES: COc1ccc2c(n1)CCCCCc1cc(F)ccc1N1CN2C(=O)c2ccc(C(F)(F)F)nc21]

Following the procedure outlined in Example 7, Step F, stirring in EtOH/EtOAc (1:0.6) at room temperature for 24 h, 3⁴-fluoro-1⁶-methoxy-2⁷-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-2⁴-one 22f (26 mg, 8% yield) was prepared as a white solid. HPLC/MS 1.32 min (A), [M+H]⁺ 487.2. ¹H NMR (CDCl₃, 400 MHz) δ 8.51 (d, J=7.8 Hz, 1H), 7.30-7.48 (m, 1H), 7.20-7.30 (m, 1H), 7.10 (dd, J=8.3, 5.4 Hz, 1H), 6.91-7.05 (m, 2H), 6.63 (d, J=8.8 Hz, 1H), 5.5-5.8 (m, 1H), 4.73 (d, J=10.8 Hz, 1H), 3.82-3.99 (m, 3H), 2.67-2.86 (m, 2H), 2.30-2.51 (m, 1H), 2.61 (dt, J=13.9, 7.2 Hz, 1H), 1.72-1.92 (m, 2H), 1.60-1.70 (m, 1H), 1.43-1.55 (m, 1H), 1.18-1.41 (m, 2H).

### Step G: 3⁴-Fluoro-2⁷-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione

### Example 22

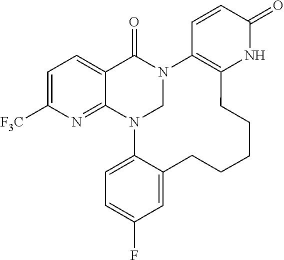 [SMILES: O=C1c2ccc(C(F)(F)F)nc2N2CN1c1ccc(=O)[nH]c1CCCCCc1cc(F)ccc12]

Following the procedure outlined in Example 7, and using Step G, stirring the reaction mixture at 100° C. for 3 h, 3⁴-fluoro-2⁷-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclo octaphane-1²,2⁴-dione (15 mg, 48% yield) was prepared as a white solid. HPLC/MS 1.00 min (A), [M+H]⁺ 473.2. ¹H NMR (DMSO-d₆, 400 MHz) δ 8.46 (d, J=7.83 Hz, 1H), 7.37-7.45 (m, 2H), 7.29-7.37 (m, 1H), 7.09-7.22 (m, 2H), 6.24 (br d, J=9.78 Hz, 1H), 5.87 (d, J=11.25 Hz, 1H), 4.84 (d, J=11.25 Hz, 1H), 2.30-2.42 (m, 2H), 1.70-1.82 (m, 1H), 1.57-1.70 (m, 1H), 1.32-1.51 (m, 2H).

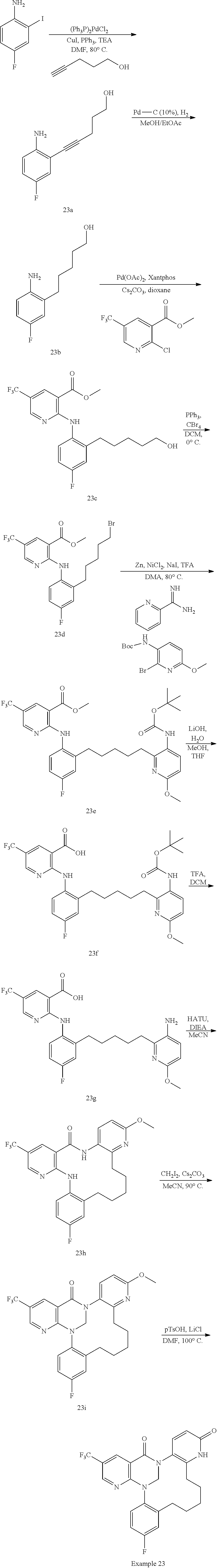

### Example 23

### 3⁴-Fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione

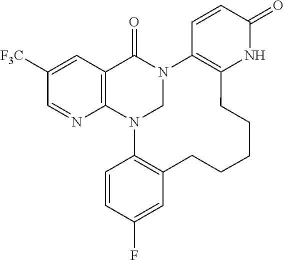 [SMILES: O=C1c2cc(C(F)(F)F)cnc2N2CN1c1ccc(=O)[nH]c1CCCCCc1cc(F)ccc12]

### Step A: 5-(2-Amino-5-fluorophenyl)pent-4-yn-1-ol 23a

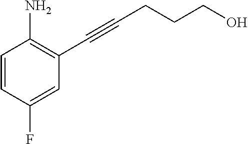 [SMILES: Nc1ccc(F)cc1C#CCCCO]

To a solution of 4-fluoro-2-iodoaniline (10.17 g, 42.9 mmol) in DMF (200 mL) was added triphenylphosphine (2.25 g, 8.58 mmol), copper iodide (1.63 g, 8.58 mmol), bis(triphenylphosphine)palladium(II) chloride (3.01 g, 4.29 mmol) and triethylamine (17.9 mL, 129 mmol). Nitrogen was bubbled through solution for 5 min, to which was added pent-4-yn-1-ol (5.99 mL, 64.4 mmol) and the reaction mixture heated at 80° C. for 15 h. The reaction mixture was cooled to ambient temperature, diluted with EtOAc and H₂O, the layers separated, and the aqueous layer was extracted with EtOAc. The combined organic extracts were washed with brine, dried over MgSO₄, and the solvent evaporated under reduced pressure. The residue was dissolved in DCM, adsorbed onto a silica gel precolumn and purified by silica gel flash column chromatography (330 g) eluting with a 100% heptanes to 100% EtOAc gradient. The product fractions were combined and evaporated under reduced pressure to afford 5-(2-amino-5-fluorophenyl)pent-4-yn-1-ol 23a (4.77 g, 52% yield) as a yellow oil. HPLC/MS 0.73 min (B), [M+H]⁺ 194.0. ¹H NMR (CDCl₃, 400 MHz) δ 6.95 (dd, J=9.3, 2.9 Hz, 1H), 6.81 (dd, J=8.8, 2.9 Hz, 1H), 6.62 (dd, J=8.8, 4.9 Hz, 1H), 3.80 (br t, J=5.8 Hz, 2H), 2.57-2.63 (m, 2H), 1.87 (br t, J=6.6 Hz, 2H).

### Step B: 5-(2-Amino-5-fluorophenyl)pentan-1-ol 23b

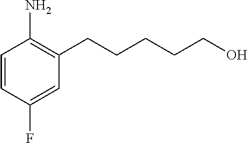 [SMILES: Nc1ccc(F)cc1CCCCCO]

A suspension of 5-(2-amino-5-fluorophenyl)pent-4-yn-1-ol (4.77 g, 24.7 mmol) in MeOH (100 mL) and EtOAc (100 mL) was sparged with N₂. Pd—C(10%) (7.88 g, 7.41 mmol) was added, the suspension was purged with N₂, a balloon of H₂ was added, the reaction vacuum/filled with H₂ and stirred at room temperature for 23 h. The reaction mixture was filtered through celite, the celite washed with MeOH, and solvent evaporated under reduced pressure. The crude residue was dissolved in DCM, adsorbed on a silica gel precolumn and purified by silica gel flash chromatography (330 g), eluting with a 20% heptanes-EtOAc to 100% EtOAc gradient. The product fractions were combined and evaporated under reduced pressure to afford 5-(2-amino-5-fluorophenyl)pentan-1-ol 23b (2.50 g, 49% yield) as a white solid. HPLC/MS 0.73 min (B), [M+H]⁺ 198.1. ¹H NMR (CDCl₃, 400 MHz) δ 6.70-6.81 (m, 2H), 6.60 (dd, J=8.6, 5.1 Hz, 1H), 3.57-3.66 (m, 2H), 2.42-2.51 (m, 2H), 1.52-1.69 (m, 4H), 1.40-1.52 (m, 2H).

### Step C: Methyl 2-((4-fluoro-2-(5-hydroxypentyl)phenyl)amino)-5-(trifluoromethyl)-nicotinate 23c

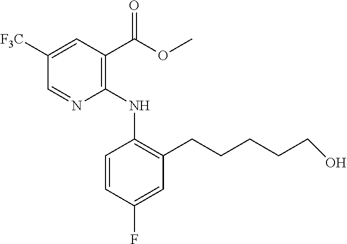 [SMILES: COC(=O)c1cc(C(F)(F)F)cnc1Nc1ccc(F)cc1CCCCCO]

Following the procedure outlined in Example 7, Step A, substituting Int-3a with 5-(2-amino-5-fluorophenyl)pentan-1-ol 23b, BINAP with Xantphos, toluene with 1,4-dioxane, and stirring the reaction mixture at room temperature for 25.5 h, methyl 2-((4-fluoro-2-(5-hydroxypentyl)phenyl)amino)-5-(trifluoromethyl)nicotinate 23c (265 mg, 41% yield) was prepared as a yellow oil. HPLC/MS 1.30 min (B), [M+H]⁺ 401.1. ¹H NMR (CDCl₃, 400 MHz) δ 10.31 (s, 1H), 8.78 (dd, J=2.5, 1.0 Hz, 1H), 8.72 (d, J=2.5 Hz, 1H), 7.98 (dd, J=8.6, 5.6 Hz, 1H), 7.20-7.29 (m, 2H), 4.26 (s, 3H), 3.88 (t, J=6.4 Hz, 2H), 2.89-2.95 (m, 2H), 1.83-2.00 (m, 5H), 1.68-1.76 (m, 2H).

### Step D: Methyl 2-((2-(5-bromopentyl)-4-fluorophenyl)amino)-5-(trifluoromethyl)-nicotinate 23d

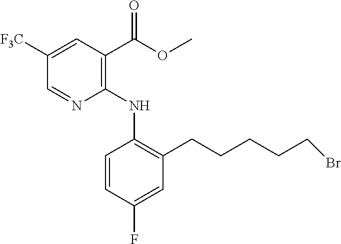 [SMILES: COC(=O)c1cc(C(F)(F)F)cnc1Nc1ccc(F)cc1CCCCCBr]

To a solution of methyl 2-((4-fluoro-2-(5-hydroxypentyl)phenyl)amino)-5-(trifluoromethyl)nicotinate (1.93 g, 4.82 mmol) in DCM (30 mL), cooled to 0° C., was added PPh₃ (1.89 g, 7.23 mmol), portion-wise, and the reaction mixture was stirred at 0° C. for 30 min, to which was added CBr₄ (4.80 g, 14.5 mmol) and the reaction mixture stirred for 1 h. The reaction mixture was adsorbed on a silica gel precolumn and purified by silica gel flash column chromatography (80 g), eluting with a 100% heptanes to 30% EtOAc-heptanes gradient. The product fractions were combined and evaporated under reduced pressure to afford methyl 2-((2-(5-bromopentyl)-4-fluorophenyl)amino)-5-(trifluoromethyl)nicotinate 23d (2.02 g, 90% yield) as a yellow solid. HPLC/MS 1.58 min (B), [M+H]⁺ 463.1, 465.1. ¹H NMR (CDCl₃, 400 MHz) δ 9.98 (s, 1H), 8.37-8.42 (m, 1H), 8.34 (d, J=2.0 Hz, 1H), 7.63 (dd, J=8.3, 5.4 Hz, 1H), 6.80-6.93 (m, 2H), 3.87 (s, 3H), 3.26 (t, J=6.6 Hz, 2H), 2.50-2.58 (m, 2H) 1.70-1.80 (m, 2H), 1.48-1.59 (m, 2H), 1.35-1.46 (m, 2H).

### Step E: Methyl 2-((2-(5-(3-((tert-butoxycarbonyl)amino)-6-methoxypyridin-2-yl)pentyl)-4-fluorophenyl)amino)-5-(trifluoromethyl)nicotinate 23e

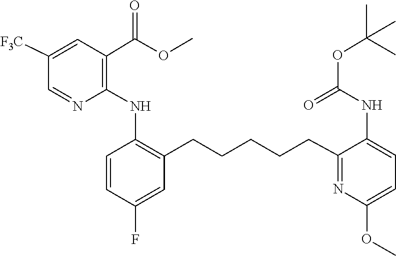 [SMILES: COC(=O)c1cc(C(F)(F)F)cnc1Nc1ccc(F)cc1CCCCCc1nc(OC)ccc1NC(=O)OC(C)(C)C]

To a 100 mL round bottom flask was added DMA (30 mL), picolinimidamide (0.026 g, 0.216 mmol), NiCl₂ (DME) (0.047 g, 0.216 mmol), NaI (0.162 g, 1.08 mmol), tert-butyl (2-bromo-6-methoxypyridin-3-yl)carbamate (0.813 g, 2.68 mmol), methyl 2-((2-(5-bromopentyl)-4-fluorophenyl)amino)-5-(trifluoromethyl)nicotinate (2.00 g, 4.32 mmol), Zn (0.565 g, 8.63 mmol) and TFA (0.033 mL, 0.432 mmol). The flask was placed under nitrogen and heated at 80° C. for 20 h. The reaction mixture was cooled to ambient temperature, diluted with EtOAc and 5% aq. NH₄Cl, filtered over celite, the phases separated, the aqueous layer was extracted with EtOAc, and the combined organic extracts washed with brine, dried over MgSO₄, filtered and the solvent evaporated under reduced pressure. The crude residue was dissolved in DCM, adsorbed on a silica gel precolumn and purified by silica gel flash column chromatography (120 g), eluting with a 100% heptanes to 100% EtOAc gradient. The product fractions were combined and evaporated under reduced pressure to afford methyl 2-((2-(5-(3-((tert-butoxycarbonyl)amino)-6-methoxypyridin-2-yl)pentyl)-4-fluorophenyl)amino)-5-(trifluoromethyl) nicotinate 23e (0.96 g, 35% yield) as a yellow oil. HPLC/MS 1.67 min (B), [M+H]⁺ 607.1. ¹H NMR (CDCl₃, 400 MHz) δ 10.34 (s, 1H), 8.79 (d, J=2.0 Hz, 1H), 8.71 (d, J=2.0 Hz, 1H), 7.99 (dd, J=8.3, 5.4 Hz, 2H), 7.20-7.26 (m, 2H), 6.82 (d, J=8.8 Hz, 1H), 6.43 (br s, 1H).

### Step F: 2-((2-(5-(3-((tert-Butoxycarbonyl)amino)-6-methoxypyridin-2-yl)pentyl)-4-fluorophenyl)amino)-5-(trifluoromethyl)nicotinic acid 23f

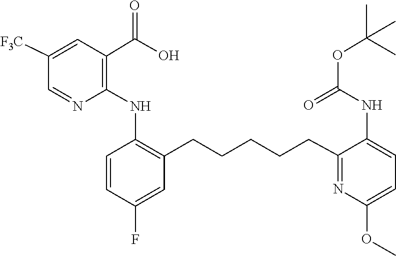 [SMILES: COc1ccc(NC(=O)OC(C)(C)C)c(CCCCCc2cc(F)ccc2Nc2ncc(C(F)(F)F)cc2C(=O)O)n1]

Following the procedure outlined in Example 7, Step B, stirring the reaction mixture in a THF/MeOH/H₂O solvent system at room temperature for 1 h, 2-((2-(5-(3-((tert-butoxycarbonyl)amino)-6-methoxypyridin-2-yl)pentyl)-4-fluorophenyl)amino)-5-(trifluoromethyl) nicotinic acid 23f (930 mg, 97% yield) was prepared as a yellow foam. HPLC/MS 1.06 min (B), [M+H]⁺ 593.2, ¹H NMR (CDCl₃, 400 MHz) δ 10.08 (s, 1H), 8.50 (br d, J=9.8 Hz, 1H), 8.45 (br s, 1H), 7.85 (br s, 1H), 7.70-7.81 (m, 1H), 7.42 (br d, J=7.8 Hz, 1H), 6.82-6.92 (m, 2H). 6.44-6.54 (m, 1H), 3.77-3.91 (m, 3H), 2.75 (br t, J=7.1 Hz, 2H), 2.47-2.58 (m, 2H), 1.81 (br dd, J=7.6, 5.6 Hz, 2H), 1.54-1.65 (m, 2H), 1.41-1.50 (m, 9H).

### Step G: 2-((2-(5-(3-Amino-6-methoxypyridin-2-yl)pentyl)-4-fluorophenyl)amino)-5-(trifluoromethyl)nicotinic acid, trifluoroacetate salt 232

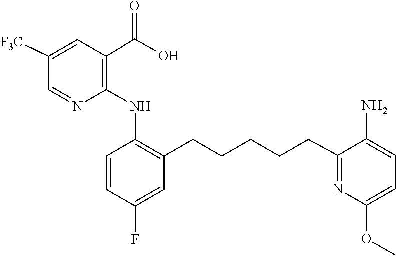 [SMILES: COc1ccc(N)c(CCCCCc2cc(F)ccc2Nc2ncc(C(F)(F)F)cc2C(=O)O)n1]

To a mixture of 2-((2-(5-(3-((tert-butoxycarbonyl)amino)-6-methoxypyridin-2-yl)pentyl)-4-fluorophenyl) amino)-5-(trifluoromethyl)nicotinic acid (930 mg, 1.57 mmol) in DCM (5 mL) was added 50% TFA in DCM (4.84 mL, 31.4 mmol) and the reaction mixture was stirred at room temperature for 2 h. The reaction mixture was evaporated under reduced pressure, azeotroped with DCM and methanol and dried under high vacuum to afford 2-((2-(5-(3-amino-6-methoxypyridin-2-yl)pentyl)-4-fluorophenyl)amino)-5-(trifluoromethyl)nicotinic acid, trifluoroacetate salt 23g (1.25 g, 100% yield) as a yellow foam. HPLC/MS 0.92 min (B), [M+H]⁺ 493.1, ¹H NMR (CD₃OD, 400 MHz) δ 8.46 (s, 1H), 8.41 (s, 1H), 7.61-7.77 (m, 2H), 6.72-7.00 (m, 3H), 5.71 (br s, 5H), 3.86-3.97 (m, 3H), 2.73 (br t, J=7.6 Hz, 1H), 2.58-2.69 (m, 3H), 1.56-1.76 (m, 4H), 1.47 (br d, J=7.3 Hz, 2H).

### Step H: 8-Fluoro-16-methoxy-2-(trifluoromethyl)-10,11,12,13,14,19-hexahydrobenzo-[m]dipyrido[2,3-b:3′,2′-f][1,5]diazacyclotetradecan-20(5H)-one 23h

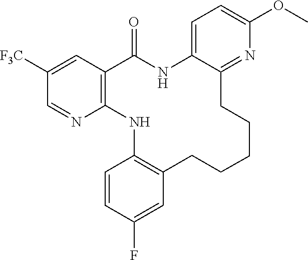 [SMILES: COc1ccc2c(n1)CCCCCc1cc(F)ccc1Nc1ncc(C(F)(F)F)cc1C(=O)N2]

Following the procedure outlined in Example 7, and using Step C, stirring the reaction mixture in MeCN at room temperature for 1 h, 8-fluoro-16-methoxy-2-(trifluoromethyl)-10,11,12,13,14,19-hexahydrobenzo [m]dipyrido[2,3-b:3′,2′-f][1,5]diazacyclotetradecin-20(5H)-one 23h (162 mg, 15% yield) was prepared as an off-white solid. HPLC/MS 1.58 min (B), [M+H]⁺ 475.1. ¹H NMR (CDCl₃, 400 MHz) δ 10.05 (s, 1H), 8.55-8.67 (m, 2H), 8.02 (d, J=2.5 Hz, 1H), 7.58 (s, 1H), 7.43 (d, J=8.3 Hz, 1H), 6.85-7.04 (m, 2H), 6.65 (d, J=8.3 Hz, 1H), 3.98 (s, 3H), 2.58-2.70 (m, 2H), 2.52 (t, J=7.6 Hz, 2H), 1.97-2.18 (m, 2H), 1.47-1.65 (m, 4H).

### Step I: 3⁴-Fluoro-1⁶-methoxy-2⁶-(trifluoromethyl)-2¹,2²,2³,2⁴-tetrahydro-2(3,1)-pyrido-[2,3-d]pyrimidina-1(3,2)-pyridina-3(1,2)-benzenacyclooctaphan-2⁴-one 23i

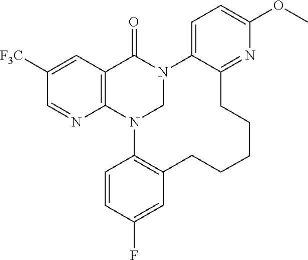 [SMILES: COc1ccc2c(n1)CCCCCc1cc(F)ccc1N1CN2C(=O)c2cc(C(F)(F)F)cnc21]

Following the procedure outlined in Example 15, Step G, stirring the reaction mixture at 90° C. for 21.5 h, 3⁴-fluoro-1⁶-methoxy-2⁶-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-2⁴-one 23i (134 mg, 75% yield) was prepared as a white solid. HPLC/MS 1.38 min (B), [M+H]⁺ 487.1. ¹H NMR (CDCl₃, 400 MHz) δ 8.61 (d, J=2.45 Hz, 1H), 8.54 (d, J=2.45 Hz, 1H), 7.42 (d, J=8.80 Hz, 1H), 7.15 (dd, J=8.8, 5.4 Hz, 1H), 7.07 (dd, J=9.3, 2.9 Hz, 1H), 7.01-7.05 (m, 1H), 6.66 (d, J=8.8 Hz, 1H), 5.76 (d, J=10.8 Hz, 1H), 4.75 (d, J=10.7 Hz, 1H), 3.93 (s, 3H), 2.72-2.87 (m, 2H), 2.67 (dt, J=14.3, 7.3 Hz, 1H), 2.43-2.56 (m, 1H), 1.79-1.96 (m, 2H), 1.62-1.70 (m, 1H), 1.50-1.61 (m, 1H), 1.26-1.44 (m, 2H).

### Step J: 3⁴-Fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphane-1²,2⁴-dione

### Example 23

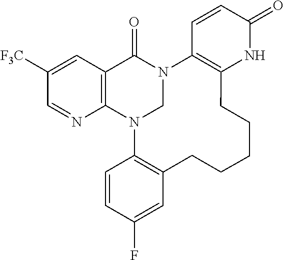 [SMILES: O=C1c2cc(C(F)(F)F)cnc2N2CN1c1ccc(=O)[nH]c1CCCCCc1cc(F)ccc12]

Following the procedure outlined in Example 15, Step J, stirring the reaction mixture at 100° C. for 16.5 h, 3⁴-fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclo octaphane-1²,2⁴-dione (75 mg, 57% yield) as a white solid. HPLC/MS 0.98 min (A), [M+H]⁺ 473.0. ¹H NMR (CDCl₃, 400 MHz) δ 8.45 (d, J=2.5 Hz, 1H), 8.39 (d, J=2.0 Hz, 1H), 7.12-7.23 (m, 1H), 7.00 (dd, J=8.6, 5.1 Hz, 1H), 6.84-6.97 (m, 2H), 6.34 (d, J=9.3 Hz, 1H), 5.59 (d, J=10.8 Hz, 1H), 4.60 (d, J=10.8 Hz, 1H), 2.42-2.64 (m, 3H), 2.24-2.42 (m, 1H), 1.69-1.89 (m, 2H), 1.37-1.50 (m, 2H), 1.24 (br dd, J=12.7, 6.4 Hz, 1H), 1.13-1.20 (m, 1H).

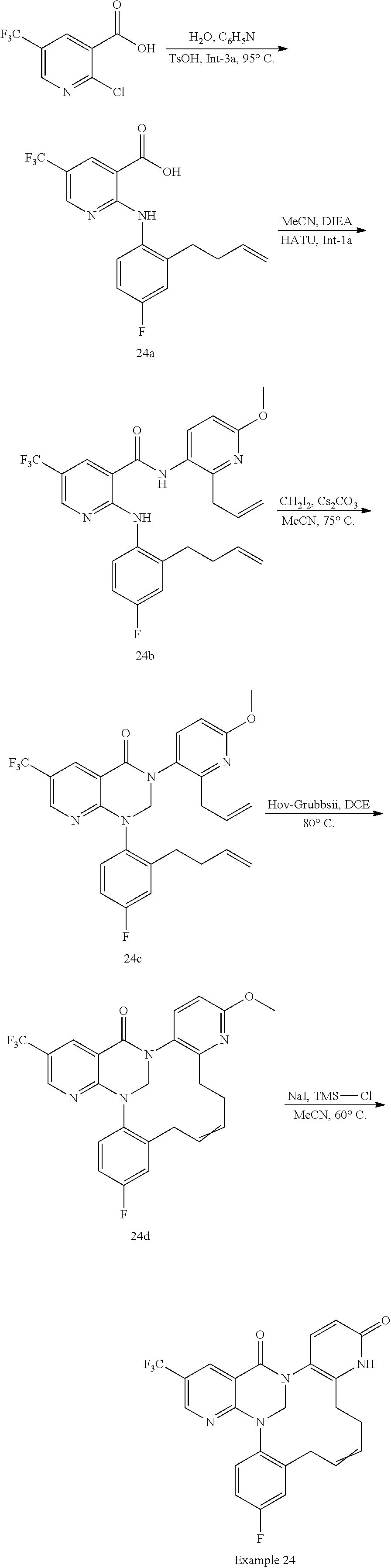

### Example 24

### 3⁴-Fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphan-5-ene-1²,2⁴-dione

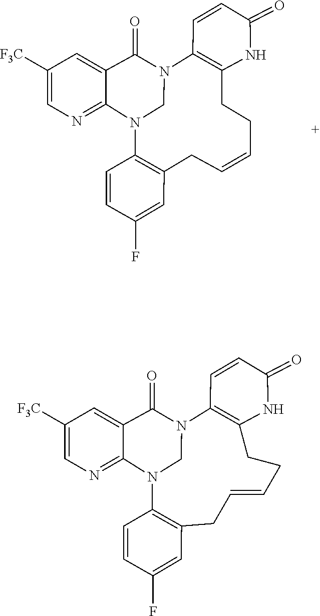 [SMILES: O=C1c2cc(C(F)(F)F)cnc2N2CN1c1ccc(=O)[nH]c1CC/C=C\Cc1cc(F)ccc12 ; O=C1c2cc(C(F)(F)F)cnc2N2CN1c1ccc(=O)[nH]c1CC/C=C/Cc1cc(F)ccc12]

### Step A: 2-((2-(But-3-en-1-yl)-4-fluorophenyl)amino)-5-(trifluoromethyl)nicotinic acid 24a

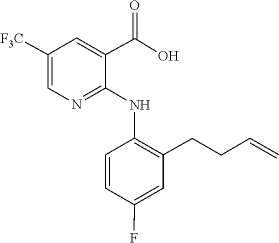 [SMILES: C=CCCc1cc(F)ccc1Nc1ncc(C(F)(F)F)cc1C(=O)O]

To a solution of 2-chloro-5-(trifluoromethyl)nicotinic acid (1.20 g, 5.32 mmol) in H₂O (10.6 ml), under N₂, was added Int-3a (0.967 g, 5.85 mmol), p-TsOH-H₂O (0.304 g, 1.59 mmol) and pyridine (0.430 ml, 5.32 mmol) and the reaction mixture stirred at 95° C. for 14.5 h. The reaction was cooled to ambient temperature, the brown solid filtered, washed with H₂O (3×), air dried and further dried under high vacuo to afford 2-((2-(but-3-en-1-yl)-4-fluorophenyl)amino)-5-(trifluoromethyl)nicotinic acid 24a (1.05 g, 78% yield). HPLC/MS 1.36 min (A), [M+H]⁺ 355.0. ¹H NMR (DMSO-d₆, 400 MHz) δ 13.7-14.3 (m, 1H), 10.37 (s, 1H), 8.6-8.7 (m, 1H), 8.39 (d, 1H, J=2.9 Hz), 7.83 (dd, 1H, J=5.6, 9.0 Hz), 7.16 (dd, 1H, J=2.9, 9.8 Hz), 7.08 (dt, 1H, J=3.2, 8.7 Hz), 5.83 (tdd, 1H, J=6.6, 10.3, 17.1 Hz), 4.9-5.0 (m, 2H), 2.69 (dd, 2H, J=6.8, 8.8 Hz), 2.3-2.4 (m, 2H).

### Step B: N-(2-Allyl-6-methoxypyridin-3-yl)-2-((2-(but-3-en-1-yl)-4-fluorophenyl)-amino)-5-(trifluoromethyl)nicotinamide 24b

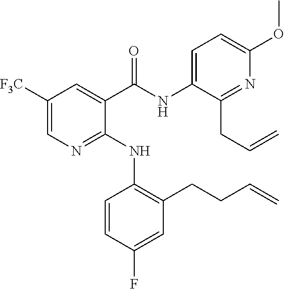 [SMILES: C=CCCc1cc(F)ccc1Nc1ncc(C(F)(F)F)cc1C(=O)Nc1ccc(OC)nc1CC=C]

To a solution of 2-((2-(but-3-en-1-yl)-4-fluorophenyl)amino)-5-(trifluoromethyl)nicotinic acid (1.50 g, 4.23 mmol) and Int-1a (0.834 g, 5.08 mmol) in MeCN (14.1 ml) was added HATU (2.42 g, 6.35 mmol) and DIEA (3.70 ml, 21.2 mmol) and the reaction mixture stirred at room temperature for 4 h. The reaction mixture was diluted with EtOAc, washed with H₂O (2×), the aqueous washes combined and extracted with EtOAc. The combined organic extracts were washed with brine, dried over MgSO₄, filtered, and the solvent evaporated under reduced pressure. The crude residue was purified by silica gel flash column chromatography (40 g) eluting with a 100% heptanes to 25% EtOAc-heptanes gradient. The product fractions were combined and evaporated under reduced pressure to afford the N-(2-allyl-6-methoxypyridin-3-yl)-2-((2-(but-3-en-1-yl)-4-fluorophenyl)amino)-5-(trifluoromethyl)nicotinamide 24b (1.72 g, 81% yield). HPLC/MS 1.50 min (A), [M+H]⁺ 501.0. ¹H NMR (DMSO-d₆, 400 MHz) δ 10.59 (s, 1H), 10.40 (s, 1H), 8.6-8.6 (m, 2H), 7.80 (dd, 1H, J=5.4, 8.8 Hz), 7.66 (d, 1H, J=8.8 Hz), 7.13 (dd, 1H, J=2.9, 9.8 Hz), 7.07 (dt, 1H, J=2.9, 8.6 Hz), 6.77 (d, 1H, J=8.8 Hz), 6.05 (tdd, 1H, J=6.7, 10.1, 17.0 Hz), 5.77 (tdd, 1H, J=6.6, 10.3, 17.1 Hz), 4.9-5.1 (m, 4H), 3.88 (s, 3H), 3.51 (td, 2H, J=1.5, 6.4 Hz), 2.65 (dd, 2H, J=6.4, 8.8 Hz), 2.2-2.3 (m, 2H).

### Step C: 3-(2-Allyl-6-methoxypyridin-3-yl)-1-(2-(but-3-en-1-yl)-4-fluorophenyl)-6-(trifluoromethyl)-2,3-dihydropyrido[2,3-d]pyrimidin-4(11H)-one 24c

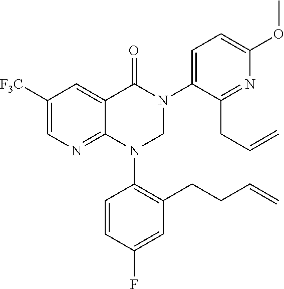 [SMILES: C=CCCc1cc(F)ccc1N1CN(c2ccc(OC)nc2CC=C)C(=O)c2cc(C(F)(F)F)cnc21]

Following the procedure outlined in Example 11, and using Step E, stirring the reaction mixture in MeCN at 70° C. for 4 h, then 75° C. for 20 h, to which was added additional CH₂I₂ (6 equiv) and the reaction continued to heat at 75° C. for 6 h, 3-(2-allyl-6-methoxypyridin-3-yl)-1-(2-(but-3-en-1-yl)-4-fluorophenyl)-6-(trifluoromethyl)-2,3-dihydropyrido[2,3-d]pyrimidin-4(1H)-one 24c (0.389 g, 91% yield) was prepared. HPLC/MS 1.46 and 1.49 min (B), [M+H]⁺ 513.0.

### Step D: 3⁴-Fluoro-1⁶-methoxy-2⁶-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-5-en-2⁴-one 24d

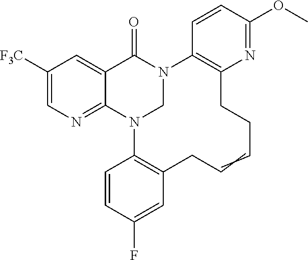 [SMILES: COc1ccc2c(n1)CCC=CCc1cc(F)ccc1N1CN2C(=O)c2cc(C(F)(F)F)cnc21]

Following the procedure outlined in Example 7, Step E, stirring the reaction mixture in DCE at 80° C. for 18 h, 3⁴-fluoro-1⁶-methoxy-2⁶-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-5-en-2⁴-one 24d (18 mg, 9% yield) was prepared. HPLC/MS 1.29-1.39 min (G), [M+H]⁺ 484.0.

### Step E: 3⁴-Fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphan-5-ene-1²,2⁴-dione

### Example 24

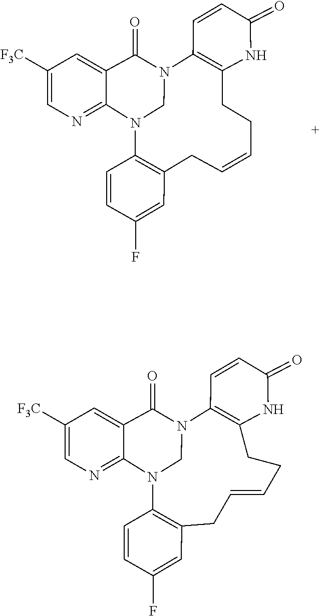 [SMILES: O=C1c2cc(C(F)(F)F)cnc2N2CN1c1ccc(=O)[nH]c1CC/C=C\Cc1cc(F)ccc12 ; O=C1c2cc(C(F)(F)F)cnc2N2CN1c1ccc(=O)[nH]c1CC/C=C/Cc1cc(F)ccc12]

To a solution of 3⁴-fluoro-1⁶-methoxy-2⁶-(trifluoromethyl)-2¹,2²,2³,2⁴-tetrahydro-2(3,1)-pyrido[2,3-d]pyrimidina-1(3,2)-pyridina-3(1,2)-benzenacyclooctaphan-5-en-2⁴-one (10 mg, 0.021 mmol) and NaI (6.19 mg, 0.041 mmol) in MeCN (206 μl) was added TMS-C₁ (5.28 μl, 0.041 mmol) and the reaction mixture heated at 60° C. for 2 h. The reaction mixture was cooled to ambient temperature and the solvent was concentrated under reduced pressure. The crude residue was purified by MDAP (XSELECT CSH C₁₈ (150 mm×30 mm 5 μm) eluting with a 30% MeCN—H₂O (0.1% formic acid) to 99% MeCN—H₂O (0.1% formic acid) gradient. Product fractions were combined, evaporated and dried under reduced pressure to afford 3⁴-fluoro-2⁶-(trifluoromethyl)-1¹,1²,2¹,2²,2³,2⁴-hexahydro-2(3,1)-pyrido[2,3-d]pyrimidina-1(5,6)-pyridina-3(1,2)-benzenacyclooctaphan-5-ene-1²,2⁴-dione (4 mg, 40% yield). HPLC/MS 0.97 min (A), [M+H]⁺ 471.0.

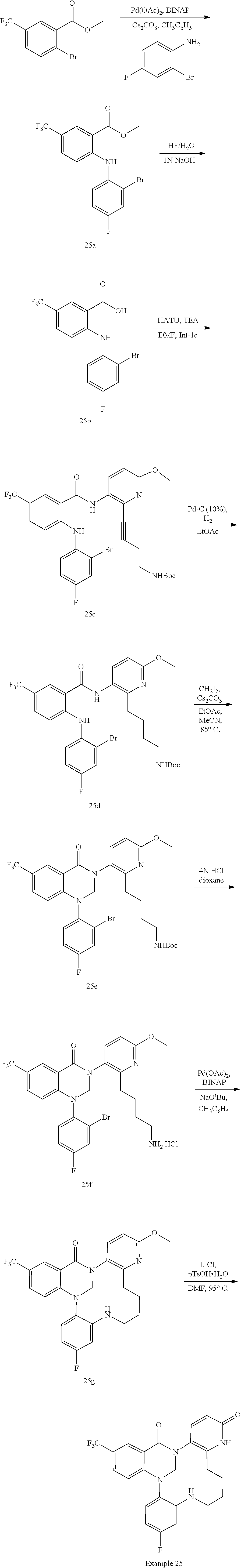

### Example 25

### 3⁴-Fluoro-2⁶-(trifluoromethyl)-1¹,1²,2¹,2²,2³,2⁴-hexahydro-4-aza-2(3,1)-quinazolina-1(5,6)-pyridina-3(1,2)-benzenacyclooctaphan-1²,2⁴-dione

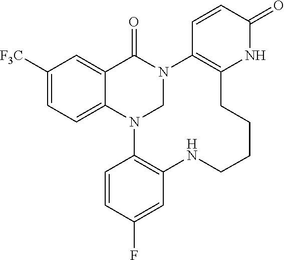 [SMILES: O=C1c2cc(C(F)(F)F)ccc2N2CN1c1ccc(=O)[nH]c1CCCCNc1cc(F)ccc12]

### Step A: Methyl 2-((2-bromo-4-fluorophenyl)amino)-5-(trifluoromethyl)benzoate 25a

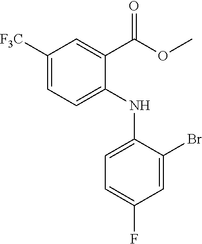 [SMILES: COC(=O)c1cc(C(F)(F)F)ccc1Nc1ccc(F)cc1Br]

Following the procedure outlined in Example 7, Step A, substituting Int-3a with 2-bromo-4-fluoroaniline, ethyl 2-bromo-5-(trilfuoromethyl)benzoate with Int-2, and stirring the reaction mixture at 100° C. for 12 h, methyl 2-((2-bromo-4-fluorophenyl)amino)-5-(trifluoromethyl)benzoate 25a (523 mg, 24% yield) was prepared as a white solid. HPLC/MS 1.48 min (A), [M+H]⁺ 391.0. ¹H NMR (DMSO-d₆, 400 MHz) δ 9.62 (s, 1H), 8.1-8.2 (m, 1H), 7.78 (dd, 1H, J=2.9, 8.3 Hz), 7.69 (dd, 1H, J=2.4, 8.8 Hz), 7.58 (dd, 1H, J=5.9, 8.8 Hz), 7.36 (dt, 1H, J=2.9, 8.6 Hz), 6.87 (d, 1H, J=8.8 Hz), 3.92 (s, 3H).

### Step B: 2-((2-Bromo-4-fluorophenyl)amino)-5-(trifluoromethyl)benzoate 25b

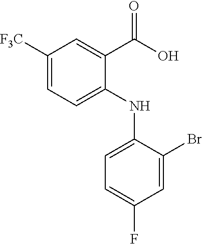 [SMILES: O=C(O)c1cc(C(F)(F)F)ccc1Nc1ccc(F)cc1Br]

Following the procedure outlined in Example 7, Step B, substituting LiOH with 1M NaOH and stirring the reaction mixture at room temperature for 72 h, to which was added additional 1M NaOH (1 equiv), and the reaction mixture continued to stir at room temperature for 5 h, then 60° C. for 2 h, and room temperature for 48 h, 2-((2-bromo-4-fluorophenyl)amino)-5-(trifluoromethyl)benzoic acid 25b (560 mg, >100% yield) was prepared as a beige solid. HPLC/MS 0.82 min (A), [M+H]⁺ 378.9. ¹H NMR (DMSO-d₆, 400 MHz) δ 13.70 (br d, 1H, J=2.0 Hz), 9.92 (br s, 1H), 8.16 (d, 1H, J=2.0 Hz), 7.77 (dd, 1H, J=2.9, 8.3 Hz), 7.67 (dd, 1H, J=1.7, 9.0 Hz), 7.6-7.6 (m, 1H), 7.3-7.4 (m, 1H), 6.9-6.9 (m, 1H).

### Step C: tert-Butyl (4-(3-(2-((2-bromo-4-fluorophenyl)amino)-5-(trifluoromethyl-benzamido)-6-methoxy pyridin-2-yl)but-3-yn-1-yl)carbamate 25c

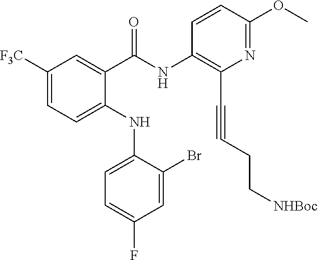 [SMILES: COc1ccc(NC(=O)c2cc(C(F)(F)F)ccc2Nc2ccc(F)cc2Br)c(C#CCCNC(=O)OC(C)(C)C)n1]

Following the procedure outlined in Example 7, Step C, substituting Int-1a with Int-1c, and stirring the reaction mixture at room temperature for 19 h, tert-butyl (4-(3-(2-((2-bromo-4-fluorophenyl)amino)-5-(trifluoromethyl)benzamido)-6-methoxypyridin-2-yl)but-3-yn-1-yl)carbamate 25c (101 mg, 24% yield) was prepared as a yellow semi-solid. HPLC/MS 1.57 min (A), [M+H]⁺ 651.0. ¹H NMR (CD₂Cl₂, 400 MHz) δ 9.6-9.9 (m, 1H), 8.3-8.6 (m, 1H), 7.9-8.0 (m, 1H), 7.5-7.6 (m, 1H), 7.4-7.5 (m, 2H), 7.1-7.2 (m, 2H), 6.8-6.9 (m, 1H), 3.9-4.1 (m, 3H), 3.47 (q, 1H, J=6.0 Hz), 3.2-3.4 (m, 1H), 2.7-2.8 (m, 2H), 2.47 (br d, 1H, J=6.8 Hz), 1.3-1.4 (m, 9H).

### Step D: tert-Butyl (4-(3-(2-((2-bromo-4-fluorophenyl)amino)-5-(trifluoromethyl)-benzamido)-6-methoxypyridin-2-yl)butyl)carbamate 25d

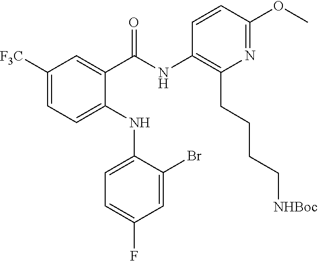 [SMILES: COc1ccc(NC(=O)c2cc(C(F)(F)F)ccc2Nc2ccc(F)cc2Br)c(CCCCNC(=O)OC(C)(C)C)n1]

Following the procedure outlined in Example 7, Step F, substituting MeOH with EtOAc, and stirring the reaction mixture at room temperature for 1 h, tert-butyl (4-(3-(2-((2-bromo-4-fluorophenyl)amino)-5-(trifluoromethyl)benzamido)-6-methoxypyridin-2-yl)butyl)carbamate 25d (87 mg, 77% yield) was prepared as a yellow solid. HPLC/MS 1.55 min (A), [M+H]⁺ 655.0. ¹H NMR (CD₃CN, 400 MHz) δ 9.75 (s, 1H), 8.81 (br s, 1H), 8.0-8.2 (m, 1H), 7.55 (d, 2H, J=8.8 Hz), 7.4-7.5 (m, 2H), 7.13 (ddd, 1H, J=2.9, 8.1, 9.0 Hz), 7.04 (d, 1H, J=8.8 Hz), 6.61 (d, 1H, J=8.8 Hz), 5.23 (br s, 1H), 3.87 (s, 3H), 2.98 (q, 2H, J=6.8 Hz), 2.67 (t, 2H, J=7.3 Hz), 1.69 (t, 2H, J=7.6 Hz), 1.40 (br t, 2H, J=7.1 Hz), 1.30 (s, 9H).

### Step E: tert-Butyl (4-(3-(1-(2-bromo-4-fluorophenyl)-4-oxo-6-(trifluoromethyl)-1,4-dihydroquinazolin-3(2H)-yl)-6-methoxypyridin-2-yl)butyl)carbamate 25e

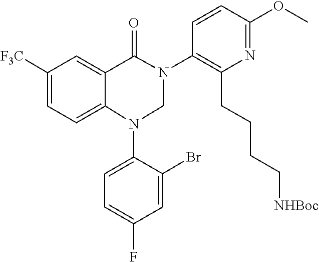 [SMILES: COc1ccc(N2CN(c3ccc(F)cc3Br)c3ccc(C(F)(F)F)cc3C2=O)c(CCCCNC(=O)OC(C)(C)C)n1]

Following the procedure outlined in Example 11, Step E, stirring the reaction mixture at 85° C. for 14 h, tert-butyl (4-(3-(1-(2-bromo-4-fluorophenyl)-4-oxo-6-(trifluoromethyl)-1,4-dihydroquinazolin-3(2H)-yl)-6-methoxypyridin-2-yl)butyl)carbamate 25e (32 mg, 33% yield) was prepared as a yellow solid. HPLC/MS 1.50 min (A), [M+H]⁺ 669.1. ¹H NMR (CD₃CN, 400 MHz) δ 8.25 (s, 1H), 7.64 (br t, 2H, J=8.8 Hz), 7.5-7.6 (m, 2H), 7.31 (ddd, 1H, J=2.9, 7.8, 8.8 Hz), 6.69 (d, 1H, J=8.3 Hz), 6.4-6.6 (m, 1H), 5.3-5.6 (m, 1H), 5.22 (br s, 1H), 4.8-5.1 (m, 1H), 3.9-4.0 (m, 3H), 2.9-3.1 (m, 2H), 2.6-2.9 (m, 2H), 1.7-1.8 (m, 2H), 1.4-1.5 (m, 2H), 1.38 (s, 9H).

### Step F: 3-(2-(4-Aminobutyl)-6-methoxypyridin-3-yl)-1-(2-bromo-4-fluorophenyl)-6-(trifluoromethyl)-2,3-dihydroquinazolin-4(1H)-yl)-one, hydrochloride salt 25f

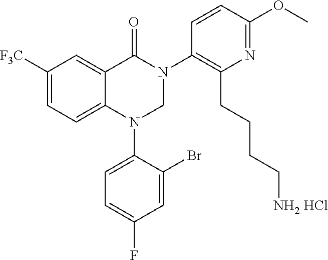

Following the procedure outlined in Example 11, Step F, stirring the reaction mixture at 60° C. for 2.5 h, then at room temperature for 13.5 h, 3-(2-(4-aminobutyl)-6-methoxypyridin-3-yl)-1-(2-bromo-4-fluorophenyl)-6-(trifluoromethyl)-2,3-dihydroquinazolin-4(1H)-yl)-one, hydrochloride salt 25f (32 mg, 33% yield) was prepared as an off-white solid. HPLC/MS 0.71 min (A), [M+H]⁺ 555.0. ¹H NMR (CD₃CN, 400 MHz) δ 8.17 (br d, 2H, J=6.8 Hz), 7.4-7.6 (m, 8H), 7.2-7.4 (m, 4H), 6.62 (d, 1H, J=8.3 Hz), 6.4-6.6 (m, 1H), 6.3-6.4 (m, 1H), 6.2-6.3 (m, 1H), 5.4-5.5 (m, 1H), 5.2-5.3 (m, 1H), 4.9-5.0 (m, 1H), 4.7-4.8 (m, 1H), 3.8-3.9 (m, 3H), 2.3-2.8 (m, 10H), 1.3-1.7 (m, 11H).

### Step G: 3⁴-Fluoro-1⁶-methoxy-2⁶-(trifluoromethyl)-2¹,2²,2³,2⁴-tetrahydro-4-aza-2(3,1)-quinazolina-1(3,2)-pyridina-3(1,2)-benzenacyclooctaphan-2⁴-one 252

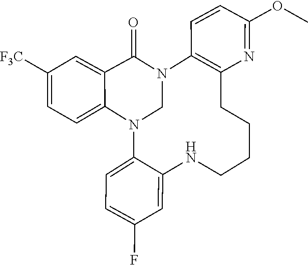 [SMILES: COc1ccc2c(n1)CCCCNc1cc(F)ccc1N1CN2C(=O)c2cc(C(F)(F)F)ccc21]

Following the procedure outlined in Example 11, Step G, stirring at 100° C. for 12 h, then at room temperature for 4 days, 3⁴-fluoro-1⁶-methoxy-2⁶-(trifluoromethyl)-2¹,2²,2³,2⁴-tetrahydro-4-aza-2(3,1)-quinazolina-1(3,2)-pyridina-3(1,2)-benzenacyclo octaphan-2⁴-one 25g (8 mg, 68% yield) was prepared as a white solid. HPLC/MS 0.89 min (A), [M+H]⁺ 487.0. ¹H NMR (CD₃CN, 400 MHz) δ 8.0-8.1 (m, 1H), 7.3-7.4 (m, 2H), 7.05 (dd, 1H, J=6.1, 8.6 Hz), 6.55 (d, 1H, J=8.8 Hz), 6.48 (d, 1H, J=8.8 Hz), 6.37 (dd, 1H, J=2.9, 11.2 Hz), 6.26 (dt, 1H, J=2.9, 8.6 Hz), 5.21 (d, 1H, J=10.8 Hz), 4.62 (br d, 1H, J=1.5 Hz), 4.27 (d, 1H, J=10.8 Hz), 3.68 (s, 3H), 3.1-3.2 (m, 1H), 2.9-3.0 (m, 1H), 2.76 (dt, 1H, J=4.4, 12.5 Hz), 2.28 (dt, 2H, J=6.1, 12.6 Hz), 1.6-1.7 (m, 1H), 1.2-1.4 (m, 2H).

### Step H: 3⁴-Fluoro-2⁶-(trifluoromethyl)-1¹,1²,2¹,2²,2³,2⁴-hexahydro-4-aza-2(3,1)-quinazolina-1(5,6)-pyridina-3(1,2)-benzenacyclooctaphan-1²,2⁴-dione Example 25

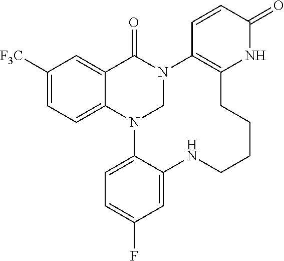 [SMILES: O=C1c2cc(C(F)(F)F)ccc2N2CN1c1ccc(=O)[nH]c1CCCCNc1cc(F)ccc12]

Following the procedure outlined in Example 11, Step H, stirring at 95° C. for 14 h, 3⁴-fluoro-2⁶-(trifluoromethyl)-1¹,1²,2¹,2²,2³,2⁴-hexahydro-4-aza-2(3,1)-quinazolina-1(5,6)-pyridina-3(1,2)-benzena cyclo octaphan-1²,2⁴-dione (3 mg, 38% yield) was prepared as a white solid. HPLC/MS 1.05 min (A), [M+H]⁺ 473.0. ¹H NMR (CD₃OD, 400 MHz) δ 8.25 (d, 1H, J=2.0 Hz), 7.65 (dd, 1H, J=2.7, 9.0 Hz), 7.59 (d, 1H, J=9.3 Hz), 7.27 (dd, 1H, J=6.1, 8.6 Hz), 6.83 (d, 1H, J=8.8 Hz), 6.62 (dd, 1H, J=2.7, 11.0 Hz), 6.50 (dt, 1H, J=2.9, 8.6 Hz), 6.45 (d, 1H, J=9.3 Hz), 5.45 (d, 1H, J=10.3 Hz), 4.58 (d, 1H, J=10.3 Hz), 3.4-3.4 (m, 1H), 3.2-3.2 (m, 1H), 2.8-2.9 (m, 1H), 2.47 (dt, 1H, J=6.1, 12.8 Hz), 1.9-2.0 (m, 2H), 1.5-1.7 (m, 2H).

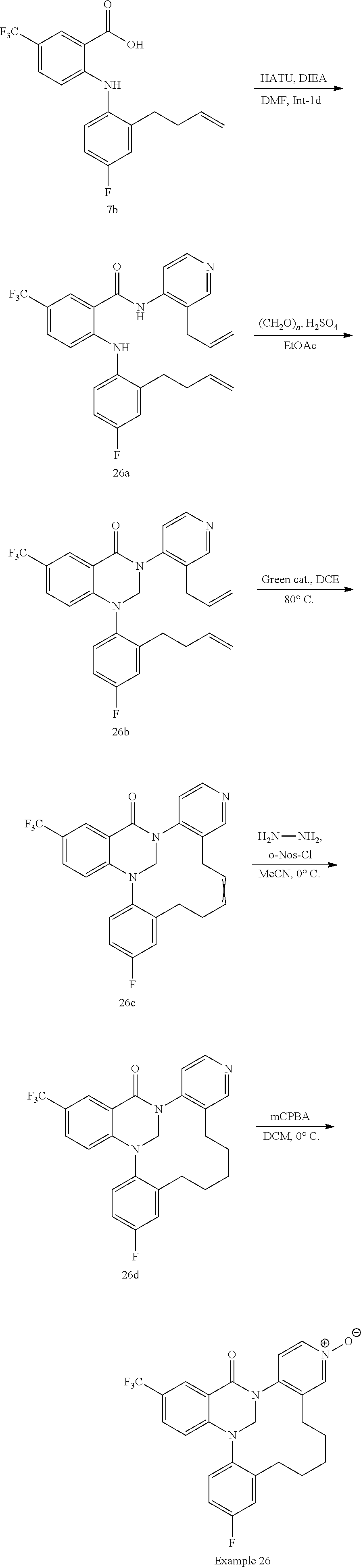

### Example 26

### 3⁴-Fluoro-2⁴-oxo-2⁶-(trifluoromethyl)-2¹,2²,2³,2⁴-tetrahydro-2(3,1)-quinazolina-1(4,3)-pyridin-1-iuma-3(1,2)-benzenacyclooctaphan-1-oxide

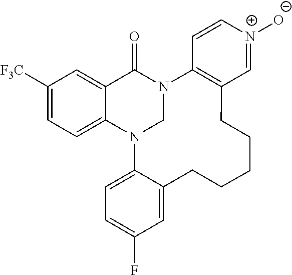 [SMILES: O=C1c2cc(C(F)(F)F)ccc2N2CN1c1cc[n+]([O-])cc1CCCCCc1cc(F)ccc12]

### Step A: N-(3-Allylpyridin-4-yl)-2-((2-(but-3-en-1-yl)-4-fluorophenyl)amino)-5-(trifluoromethyl)benzamide 26a

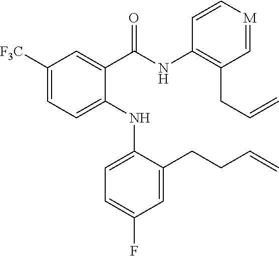

Following the procedure outlined in Example 7, Step C, substituting Int-1a with Int-1d, N-(3-allylpyridin-4-yl)-2-((2-(but-3-en-1-yl)-4-fluorophenyl)amino)-5-(trifluoromethyl)benzamide 26a (689.9 mg, 83% yield) was prepared as a viscous oil, which solidifies upon storage. HPLC/MS 1.05 min (A), [M+H]⁺ 470.2. ¹H NMR (DMSO-d₆, 400 MHz) δ 10.27 (s, 1H), 9.46 (s, 1H), 8.46 (t, 2H, J=2.7 Hz), 8.13 (d, 1H, J=1.0 Hz), 7.5-7.7 (m, 2H), 7.32 (dd, 1H, J=5.4, 8.8 Hz), 7.23 (dd, 1H, J=3.2, 9.5 Hz), 7.12 (dt, 1H, J=2.9, 8.6 Hz), 6.76 (d, 1H, J=8.8 Hz), 5.9-6.0 (m, 1H), 5.7-5.8 (m, 1H), 5.0-5.1 (m, 2H), 4.8-5.0 (m, 2H), 3.54 (d, 2H, J=6.4 Hz), 2.6-2.7 (m, 2H), 2.26 (br d, 2H, J=6.8 Hz).

### Step B: 3-(3-Allylpyridin-4-yl)-1-(2-(but-3-en-1-yl)-4-fluorophenyl)-6-(trifluoromethyl)-2,3-dihydroquinazolin-4(11H)-one 26b

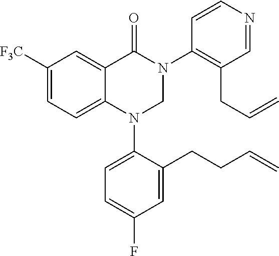 [SMILES: C=CCCc1cc(F)ccc1N1CN(c2ccncc2CC=C)C(=O)c2cc(C(F)(F)F)ccc21]

To N-(3-allylpyridin-4-yl)-2-((2-(but-3-en-1-yl)-4-fluorophenyl)amino)-5-(trifluoromethyl)benzamide (689.9 mg, 1.47 mmol) in DMF (15 mL) was added paraformaldehyde (1103 mg, 36.7 mmol), followed by pTSOH (307 mg, 1.62 mmol) and the reaction mixture was stirred at 100° C. for 3 hr. The reaction mixture was cooled to ambient temperature, diluted with EtOAc, washed with H₂O (3×), brine, dried over Na₂SO₄, filtered and the solvent evaporated under reduced pressure. The crude residue was dissolved in DCM, absorbed onto a silica gel packed precolumn and purified by silica gel flash column chromatography (80 g) eluting with a 100% heptane to 50% 3:1 EtOAc/EtOH-hexanes gradient. Pure product fractions were combined and evaporated under reduced pressure to afford 3-(3-allylpyridin-4-yl)-1-(2-(but-3-en-1-yl)-4-fluorophenyl)-6-(trifluoromethyl)-2,3-dihydroquinazolin-4(1H)-one 26b (492 mg, 68% yield) was prepared as a yellow semi-solid. HPLC/MS 1.08 min (A), [M+H]⁺ 482.2. ¹H NMR (DMSO-d₆, 400 MHz) δ 8.4-8.6 (m, 2H), 8.13 (d, 1H, J=2.0 Hz), 7.70 (dd, 1H, J=2.2, 9.0 Hz), 7.44 (dd, 1H, J=5.4, 8.8 Hz), 7.3-7.4 (m, 2H), 7.23 (br d, 1H, J=2.9 Hz), 6.38 (br d, 1H, J=8.8 Hz), 5.8-6.0 (m, 1H), 5.6-5.8 (m, 2H), 5.04 (t, 1H, J=1.5 Hz), 5.0-5.0 (m, 1H), 4.8-5.0 (m, 3H), 3.37 (br d, 2H, J=6.4 Hz), 2.6-2.7 (m, 2H), 2.32 (br s, 2H).

### Step C: 3⁴-Fluoro-2⁶-(trifluoromethyl)-2¹,2²,2³,2⁴-tetrahydro-2(3,1)-quinazolina-1(4,3)-pyridina-3(1.2)-benzenacyclooctaphan-6-en-2⁴-one 26c

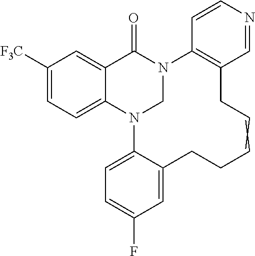 [SMILES: O=C1c2cc(C(F)(F)F)ccc2N2CN1c1ccncc1CC=CCCc1cc(F)ccc12]

Following the procedure outlined in Example 7, Step E, substituting Hoveda-Grubbsii with Greencat-iPr, stirring the reaction mixture in DCE at 80° C. for 4 h, 3⁴-fluoro-2⁶-(trifluoromethyl)-2¹,2²,2³,2⁴-tetrahydro-2(3,1)-quinazolina-1(4,3)-pyridina-3(1,2)-benzenacyclooctaphan-6-en-2⁴-one 26c (189 mg, 40% yield) was prepared as a light brown semi-solid. HPLC/MS 0.96 min (A), [M+H]⁺ 454.2. ¹H NMR (DMSO-d₆, 400 MHz) δ 8.5-8.6 (m, 2H), 8.11 (d, 1H, J=2.0 Hz), 7.6-7.8 (m, 1H), 7.37 (d, 2H, J=5.4 Hz), 7.2-7.3 (m, 2H), 6.2-6.5 (m, 1H), 5.9-6.1 (m, 1H), 5.1-5.5 (m, 2H), 4.5-4.6 (m, 1H), 3.2-3.3 (m, 2H), 2.6-2.8 (m, 2H), 2.2-2.4 (m, 1H), 1.7-2.0 (m, 1H).

### Step D: 3⁴-Fluoro-2⁶-(trifluoromethyl)-2¹,2²,2³,2⁴-tetrahydro-2(3,1)-quinazolina-1(4,3)-pyridina-3(1,2)-benzenacyclooctaphan-2⁴-one 26d

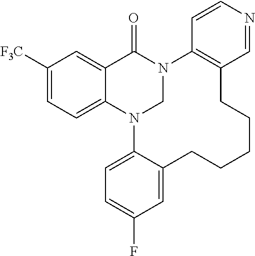 [SMILES: O=C1c2cc(C(F)(F)F)ccc2N2CN1c1ccncc1CCCCCc1cc(F)ccc12]

To a vigorously stirred solution of 3⁴-fluoro-2⁶-(trifluoromethyl)-2¹,2²,2³,2⁴-tetrahydro-2(3,1)-quinazolina-1(4,3)-pyridina-3(1,2)-benzenacyclooctaphan-6-en-2⁴-one (189 mg, 0.417 mmol), and 2-nitrobenzenesulfonyl chloride (185 mg, 0.834 mmol) in MeCN (2.0 mL), cooled to 0° C., was added hydrazine hydrate (0.163 mL, 1.67 mmol), dropwise, and the reaction mixture was stirred at 0° C. for 30 min, then allowed to slowly warm to room temperature and stirred for 22 h. Additional 2-nitrobenzenesulfonyl chloride (370 mg) was added followed by dropwise addition of cold hydrazine hydrate (330 μL) and the reaction mixture stirred for 5 h. An additional portion of 2-nitrobenzenesulfonyl chloride (370 mg) was added followed by dropwise addition of cold hydrazine hydrate (330 μL) and the reaction mixture stirred for 120 h. The reaction mixture was diluted with EtOAc, washed with H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (40 g) eluting with a 100% heptane to 100% 3:1 EtOAc-EtOH/heptanes gradient. Product fractions were combined and evaporated under reduced pressure to afford a beige solid (106.9 mg, 56% yield). This material was further purified by chiral HPLC (Sunfire, 20×150 mm, 5p), eluting with a 40% MeCN—H₂O (0.1% formic acid) to 80% MeCN—H₂O (0.1% formic acid) gradient. Product fractions were combined and lyophilized to afford 3⁴-fluoro-2⁶-(trifluoromethyl)-2¹,2²2³,2⁴-tetrahydro-2(3,1)-quinazolina-1(4,3)-pyridina-3(1,2)-benzenacyclooctaphan-2⁴-one 26d (14.8 mg, 8% yield) as a white solid. HPLC/MS 1.12 min (A), [M+H]⁺ 456.2. ¹H NMR (CD₃OD, 400 MHz) δ 8.5-8.7 (m, 2H), 8.31 (d, 1H, J=2.0 Hz), 7.69 (dd, 1H, J=2.2, 9.0 Hz), 7.51 (d, 1H, J=5.9 Hz), 7.35 (dd, 1H, J=5.4, 8.3 Hz), 7.1-7.2 (m, 2H), 6.55 (d, 1H, J=8.8 Hz), 5.92 (d, 1H, J=10.8 Hz), 5.02 (d, 1H, J=10.8 Hz), 2.8-2.9 (m, 1H), 2.7-2.8 (m, 1H), 2.63 (s, 1H), 2.4-2.5 (m, 1H), 1.91 (s, 2H), 1.5-1.7 (m, 1H), 1.44 (br dd, 1H, J=5.4, 7.3 Hz), 1.0-1.4 (m, 4H).

### Step E: 3⁴-Fluoro-2⁴-oxo-2⁶-(trifluoromethyl)-2¹,2²,2³,2⁴-tetrahydro-2(3,1)-quinazolina-1(4,3)-pyridin-1-iuma-3(1,2)-benzenacyclooctaphan-1¹-oxide Example 26

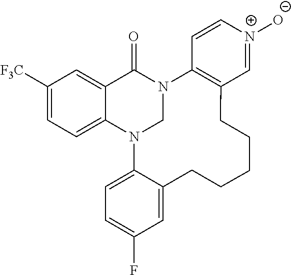 [SMILES: O=C1c2cc(C(F)(F)F)ccc2N2CN1c1cc[n+]([O-])cc1CCCCCc1cc(F)ccc12]

To a solution of 3⁴-fluoro-2⁶-(trifluoromethyl)-2¹,2²,2³,2⁴-tetrahydro-2(3,1)-quinazolina-1(4,3)-pyridina-3(1,2)-benzenacyclooctaphan-2⁴-one (14.5 mg, 0.032 mmol) in DCM (1 mL), cooled to 0° C., was added mCPBA (10.9 mg, 0.064 mmol), and the reaction mixture was stirred at 0° C. for 20 h. The reaction mixture was diluted with DCM, washed with 10% NaHCO₃ (2×), H₂O, dried over Na₂SO₄, filtered and the solvent evaporated under reduced pressure. The crude residue was dissolved in MeOH and purified by reverse-phase semi-prep HPLC (30 g Gold Aq column) eluting from 100% H₂O (0.1% formic acid) to 100% MeCN (0.1% formic acid) gradient. Product fractions were combined and evaporated under reduced pressure to afford a white solid. This material was dissolved in DMSO (1.5 mL) and purified by MDAP (XSELECT CSH C_{18, 150×30} mm, 5p) eluting with a 30% MeCN—H₂O (0.1% formic acid) to 85% MeCN—H₂O (0.1% formic acid) over 22 min. Pure product peaks were collected and evaporated under a nitrogen stream at 45° C. to afford 3⁴-fluoro-2⁴-oxo-2⁶-(trifluoromethyl)-2¹,2²,2³,2⁴-tetrahydro-2(3,1)-quinazolina-1(4,3)-pyridin-1-iuma-3(1,2)-benzenacyclooctaphan-1¹-oxide (0.4 mg) as a white solid. HPLC/MS 1.09 min (A), [M+H]⁺ 471.2. ¹H NMR (CD₃OD, 400 MHz) δ 8.1-8.4 (m, 3H), 7.68 (dd, 1H, J=2.2, 9.0 Hz), 7.54 (d, 1H, J=6.4 Hz), 7.35 (dd, 1H, J=5.1, 8.6 Hz), 7.1-7.2 (m, 2H), 6.54 (d, 1H, J=8.8 Hz), 5.84 (d, 1H, J=10.8 Hz), 5.05 (d, 1H, J=10.8 Hz), 2.73 (br d, 1H, J=2.0 Hz), 2.6-2.7 (m, 2H), 2.4-2.5 (m, 1H), 1.90 (br dd, 2H, J=7.1, 14.9 Hz), 1.5-1.7 (m, 1H), 1.4-1.5 (m, 1H), 1.1-1.4 (m, 2H).

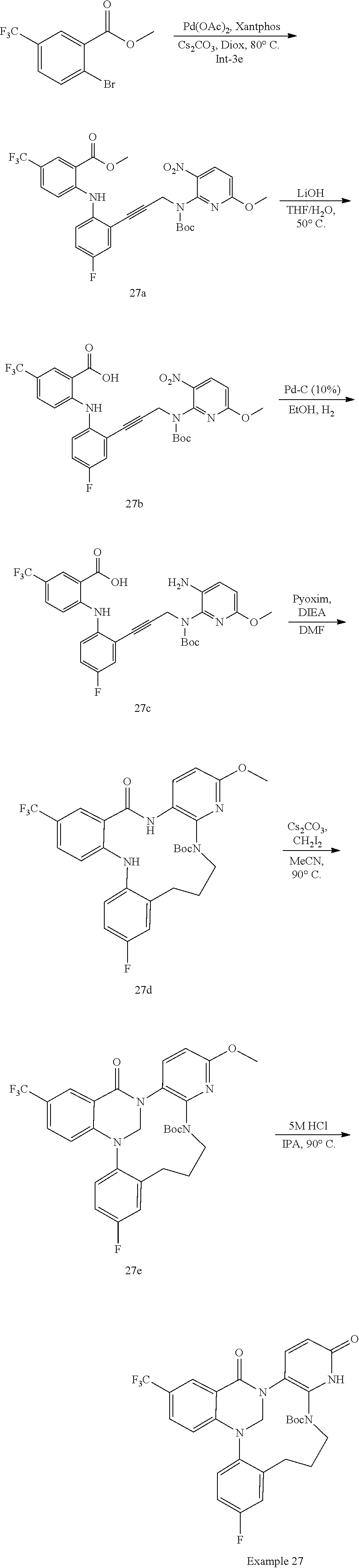

### Example 27

### 8-Fluoro-2-(trifluoromethyl)-10,11,12,13-tetrahydro-19H-5,18-methanodibenzo[f,i]pyrido[2,3-b][1,4,8]triazocyclotridecine-15,19(14H)-dione

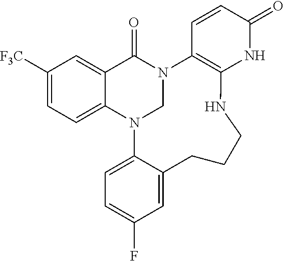 [SMILES: O=C1c2cc(C(F)(F)F)ccc2N2CN1c1ccc(=O)[nH]c1NCCCc1cc(F)ccc12]

### Step A: Methyl 2-((2-(3-((tert-butoxycarbonyl)(6-methoxy-3-nitropyridin-2-yl)amino)-prop-1-yn-1-yl)-4-fluorophenyl)amino)-5-(trifluoromethyl)benzoate 27a

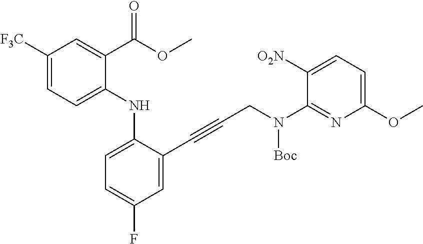 [SMILES: COC(=O)c1cc(C(F)(F)F)ccc1Nc1ccc(F)cc1C#CCN(C(=O)OC(C)(C)C)c1nc(OC)ccc1[N+](=O)[O-]]

Following the procedure outlined in Example 7, Step A, substituting Int-3a with Int-3e, stirring the reaction mixture at 80° C. for 3 h, methyl 2-((2-(3-((tert-butoxycarbonyl)(6-methoxy-3-nitropyridin-2-yl)amino)prop-1-yn-1-yl)-4-fluorophenyl)amino)-5-(trifluoromethyl)benzoate 27a (3.85 g, 52% yield) was prepared as a light yellow foam. HPLC/MS 1.62 min (A), [M+H]⁺ 619.2. ¹H NMR (DMSO-d₆, 400 MHz) δ 9.5-9.8 (m, 1H), 8.25 (s, 1H), 8.0-8.1 (m, 1H), 7.6-7.7 (m, 1H), 7.4-7.5 (m, 1H), 7.4-7.4 (m, 1H), 7.3-7.3 (m, 1H), 7.0-7.1 (m, 1H), 6.77 (d, 1H, J=8.8 Hz), 4.89 (s, 2H), 3.83 (s, 3H), 3.76 (s, 3H), 1.28 (br s, 9H).

### Step B: 2-((2-(3-tert-Butoxycarbonyl)(6-methoxy-3-nitropyridin-2-yl)amino)prop-1-yn-1-yl)-4-fluorophenyl)amino)-5-(trifluoromethyl)benzoic acid 27b

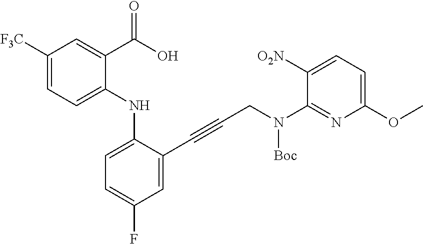 [SMILES: COc1ccc([N+](=O)[O-])c(N(CC#Cc2cc(F)ccc2Nc2ccc(C(F)(F)F)cc2C(=O)O)C(=O)OC(C)(C)C)n1]

Following the procedure outlined in Example 7, Step B, stirring the reaction mixture at 50° C. for 20 h, 2-((2-(3-tert-butoxycarbonyl)(6-methoxy-3-nitropyridin-2-yl)amino)prop-1-yn-1-yl)-4-fluorophenyl)amino)-5-(trifluoromethyl)benzoic acid 27b (2.18 g, 56% yield) was prepared as a light yellow foam. HPLC/MS 1.51 min (A), [M+H]⁺ 605.1. ¹H NMR (DMSO-d₆, 400 MHz) δ 13.3-13.7 (m, 1H), 9.99 (br s, 1H), 8.27 (d, 1H, J=8.8 Hz), 8.08 (d, 1H, J=2.0 Hz), 7.62 (dd, 1H, J=2.2, 9.0 Hz), 7.49 (dd, 1H, J=5.4, 8.8 Hz), 7.2-7.4 (m, 2H), 7.04 (d, 1H, J=8.8 Hz), 6.77 (d, 1H, J=9.3 Hz), 4.88 (s, 2H), 3.86 (s, 3H), 1.30 (br s, 9H).

### Step C: 2-((2-(3-((3-Amino-6-methoxypyridin-2-yl)(tert-butoxycarbonyl)amino)propyl)-4-fluorophenyl) amino)-5-(trifluoromethyl)benzoic acid 27c

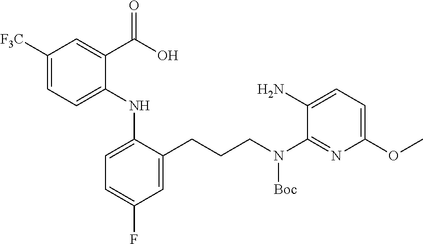 [SMILES: COc1ccc(N)c(N(CCCc2cc(F)ccc2Nc2ccc(C(F)(F)F)cc2C(=O)O)C(=O)OC(C)(C)C)n1]

To a solution of 2-((2-(3-((tert-butoxycarbonyl)(6-methoxy-3-nitropyridin-2-yl)amino)prop-1-yn-1-yl)-4-fluorophenyl)amino)-5-(trifluoromethyl)benzoic acid (2.13 g, 3.52 mmol), in EtOH (50 mL), sparged with N₂ (3×), was added Pd—C (10%) (0.375 g, 0.352 mmol), the reaction mixture sparged with N₂, then replaced with a balloon of H₂, sparged (3×), and stirred under an atmosphere of H₂ at room temperature for 3.5 h. The reaction mixture was flushed with N₂, filtered through a pad of celite, washed with EtOAc, and the solvent evaporated under reduced pressure to afford 2-((2-(3-((3-amino-6-methoxypyridin-2-yl)(tert-butoxycarbonyl)amino)propyl)-4-fluorophenyl)amino)-5-(trifluoromethyl)benzoic acid 27c (1.99 g, 95% yield) as a beige foam. HPLC/MS 1.28 min (A), [M+H]⁺ 579.2.

### Step D: tert-Butyl 8-fluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate 27d

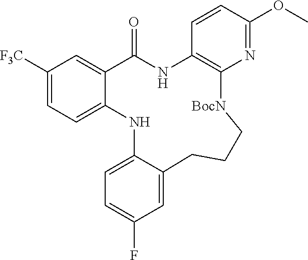 [SMILES: COc1ccc2c(n1)N(C(=O)OC(C)(C)C)CCCc1cc(F)ccc1Nc1ccc(C(F)(F)F)cc1C(=O)N2]

To a solution of 2-((2-(3-((3-amino-6-methoxypyridin-2-yl)(tert-butoxycarbonyl)amino)propyl)-4-fluorophenyl)amino)-5-(trifluoromethyl)benzoic acid (1.93 g, 3.34 mmol), in DMF (35 ml) was added pyoxim (3.17 g, 6.00 mmol), followed by DIEA (1.75 ml, 10.0 mmol), and the reaction mixture stirred at room temperature for 2 h. The reaction mixture was diluted with EtOAc, the mixture washed with H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (220 g) eluting with a 100% heptane to 30% EtOAc-heptanes gradient. Product fractions were combined and evaporated under reduced pressure to afford tert-butyl 8-fluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate 27d (1.49 g, 80% yield) as a light yellow foam. HPLC/MS 1.54 min (A), [M+H]⁺ 561.1. ¹H NMR (DMSO-d₆, 400 MHz) δ 8.7-9.2 (m, 1H), 8.36 (br s, 2H), 7.8-8.2 (m, 1H), 7.60 (br d, 1H, J=8.3 Hz), 7.30 (br dd, 2H, J=2.9, 9.8 Hz), 7.13 (dt, 1H, J=3.2, 8.4 Hz), 6.89 (d, 1H, J=8.8 Hz), 6.63 (d, 1H, J=8.8 Hz), 3.9-4.1 (m, 1H), 3.83 (s, 3H), 2.6-2.9 (m, 1H), 1.4-1.8 (m, 2H), 0.9-1.3 (m, 10H).

### Step E: tert-Butyl 8-fluoro-15-methoxy-19-oxo-2-(trifluoromethyl)-11,12-dihydro-19H-5,18-methano dibenzo[f,i]pyrido[2,3,-b][1,4,8]triazacyclotridecine-13(10H)-carboxylate 27e

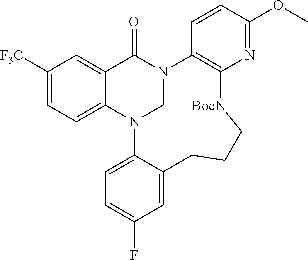 [SMILES: COc1ccc2c(n1)N(C(=O)OC(C)(C)C)CCCc1cc(F)ccc1N1CN2C(=O)c2cc(C(F)(F)F)ccc21]

Batch 1: To a solution of tert-butyl 8-fluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate (50.4 mg, 0.09 mmol), in MeCN (1 mL) was added Cs₂CO₃ (176 mg, 0.539 mmol), followed by diiodomethane (0.109 mL, 1.35 mmol) and the reaction mixture was stirred at 90° C. for 24 h. The reaction mixture was cooled to ambient temperature. Batch 2: To a solution of tert-butyl 8-fluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate (1.42 g, 2.53 mmol), in MeCN (25 mL) was added Cs₂CO₃ (4.95 g, 15.20 mmol), followed by diiodomethane (3.06 mL, 38.0 mmol) and the reaction mixture was stirred at 90° C. for 23 h. The reaction mixture was cooled to ambient temperature, combined with batch 1, and the combined reaction mixture diluted with EtOAc, washed with H₂O, brine, dried over Na₂SO₄, filtered and the solvent evaporated under reduced pressure. The crude residue was dissolved in DCM, absorbed onto a silica gel packed precolumn and purified by flash column chromatogarphy (120 g) eluting with a 100% heptane to 50% EtOAc-heptanes gradient. Product fractions were combined and evaporated under reduced pressure to afford tert-butyl 8-fluoro-15-methoxy-19-oxo-2-(trifluoromethyl)-11,12-dihydro-19H-5,18-methanodibenzo[f,i]pyrido[2,3,-b][1,4,8]triazacyclotridecine-13(10H)-carboxylate 27e (1.42 g, 95% yield) as a white foam. HPLC/MS 1.45 min (A), [M+H]⁺ 573.1. ¹H NMR (CD₃OD, 400 MHz) δ 8.31 (d, 1H, J=2.0 Hz), 7.83 (d, 1H, J=8.8 Hz), 7.59 (dd, 1H, J=2.4, 8.8 Hz), 7.41 (dd, 1H, J=5.4, 8.8 Hz), 7.32 (dd, 1H, J=2.9, 9.3 Hz), 7.10 (dt, 1H, J=3.2, 8.4 Hz), 6.86 (d, 1H, J=8.3 Hz), 6.6-6.7 (m, 1H), 5.38 (d, 1H, J=11.2 Hz), 4.61 (br s, 1H)), 3.93 (s, 5H), 3.4-3.5 (m, 1H), 2.6-2.7 (m, 1H), 2.03 (s, 1H), 1.4-1.6 (m, 1H), 1.19 (s, 9H).

### Step F: 8-Fluoro-2-(trifluoromethyl)-10,11,12,13-tetrahydro-19H-5,18-methanodibenzo-[f,i]pyrido[2,3-b][1,4,8]triazocyclotridecine-15,19(14H)-dione Example 27

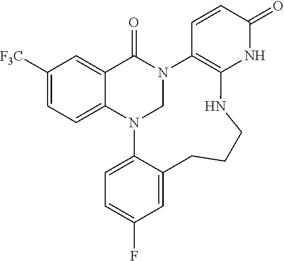 [SMILES: O=C1c2cc(C(F)(F)F)ccc2N2CN1c1ccc(=O)[nH]c1NCCCc1cc(F)ccc12]

To a solution of tert-butyl 8-fluoro-15-methoxy-19-oxo-2-(trifluoromethyl)-11,12-dihydro-19H-5,18-methanodibenzo[f,i]pyrido[2,3,-b][1,4,8]triazacyclotridecine-13(10H)-carboxylate (1.42 g, 2.48 mmol), in ⁱPrOH (20 mL) was added a 5N HCl in ⁱPrOH solution (24.8 mL, 124 mmol) and the reaction mixture was stirred at 90° C. for 120 h. The reaction mixture was cooled to ambient temperature and the solvent evaporated under reduced pressure. EtOAc was added to the residual foam, the mixture triturated, filtered, washed with EtOAc and diethyl ether, and dried under vacuo to afford an off-white solid. The solid was dissolved in MeOH and purified by semi-prep reverse-phase HPLC (C-18 Aq Gold, 150 g) eluting with a 10% MeCN—H₂O (0.1% formic acid) to 100% MeCN (0.1% formic acid) gradient. Product peaks were combined and concentrated under reduced pressure until only aqueous present. The white suspension was filtered, washed with H₂O, chased with diethyl ether and dried under vacuo to afford 8-fluoro-2-(trifluoromethyl)-10,11,12,13-tetrahydro-19H-5,18-methanodibenzo[f,i]pyrido[2,3-b][1,4,8]triazocyclo tridecine-15,19(14H)-dione (813 mg, 71% yield) as a white solid. HPLC/MS 0.99 min (A), [M+H]⁺ 459.2. ¹H NMR (DMSO-d₆, 400 MHz) δ 10.79 (br s, 1H), 8.12 (d, 1H, J=2.0 Hz), 7.65 (dd, 1H, J=2.4, 8.8 Hz), 7.46 (dd, 1H, J=5.6, 8.6 Hz), 7.3-7.4 (m, 2H), 7.22 (dt, 1H, J=2.9, 8.6 Hz), 6.42 (d, 1H, J=8.8 Hz), 5.7-5.9 (m, 2H), 5.51 (d, 1H, J=11.2 Hz), 4.50 (d, 1H, J=11.2 Hz), 3.37 (br d, 1H, J=5.9 Hz), 2.9-3.1 (m, 1H), 2.6-2.8 (m, 1H), 2.5-2.6 (m, 1H), 1.8-2.1 (m, 1H), 1.3-1.5 (m, 1H).

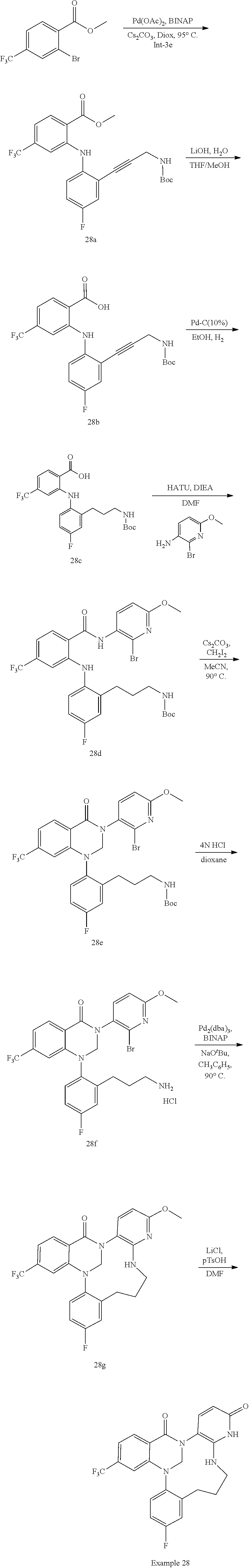

### Example 28

### 8-Fluoro-3-(trifluoromethyl)-10,11,12,13-tetrahydro-19H-5,18-methanodibenzo[f,i]pyrido[2,3-b][1,4,8]triazocyclotridecine-15,19(14H)-dione

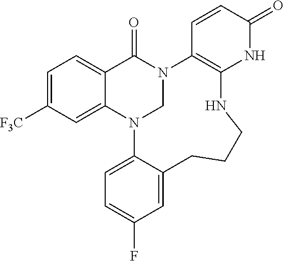 [SMILES: O=C1c2ccc(C(F)(F)F)cc2N2CN1c1ccc(=O)[nH]c1NCCCc1cc(F)ccc12]

### Step A: Methyl 2-((2-(3-((tert-butoxycarbonyl)amino)prop-1-yn-1-yl)-4-fluorophenyl)-amino)-4-(trifluoromethyl)benzoate 28a

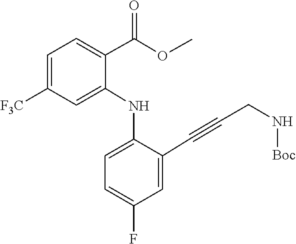 [SMILES: COC(=O)c1ccc(C(F)(F)F)cc1Nc1ccc(F)cc1C#CCNC(=O)OC(C)(C)C]

Following the procedure outlined in Example 7, Step A, substituting methyl 2-bromo-5-(trifluoromethyl)benzoate with methyl 2-bromo-4-(trifluoromethyl)benzoate, Int-3a with Int-3f, toluene with 1,4-dioxane, and stirring the reaction mixture at 95° C. for 36 h, methyl 2-((2-(3-((tert-butoxycarbonyl)(6-methoxy-3-nitropyridin-2-yl)amino)prop-1-yn-1-yl)-4-fluorophenyl)amino)-4-(trifluoromethyl)benzoate 28a (660 mg, 40% yield) was prepared as a light yellow oil. HPLC/MS 1.55 min (G), [M+H]⁺ 467.2. ¹H NMR (DMSO-d₆, 400 MHz) δ 10.11 (s, 1H), 8.12 (d, J=8.31 Hz, 1H), 7.57 (s, 1H), 7.41 (m, 1H), 7.14-7.27 (m, 1H), 6.98-7.13 (m, 2H), 5.42 (br s, 1H), 4.22 (br d, J=4.89 Hz, 2H), 3.93-4.07 (m, 3H), 1.41-1.57 (m, 9H).

### Step B: 2-((2-(3-tert-Butoxycarbonyl)amino)prop-1-yn-1-yl)-4-fluorophenyl)amino)-4-(trifluoromethyl) benzoic acid 28b

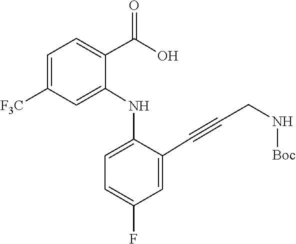 [SMILES: CC(C)(C)OC(=O)NCC#Cc1cc(F)ccc1Nc1cc(C(F)(F)F)ccc1C(=O)O]

Following the procedure outlined in Example 7, Step B, stirring the reaction mixture in a THF/MeOH/H₂O solvent mixture at ambient temperature overnight 2-((2-(3-tert-butoxycarbonyl)(6-methoxy-3-nitropyridin-2-yl)amino)prop-1-yn-1-yl)-4-fluorophenyl)amino)-4-(trifluoromethyl)benzoic acid 28b (580 mg, 92% yield) was prepared as a yellow solid. HPLC/MS 1.39 min (A), [M+H]⁺ 453.1. ¹H NMR (CDCl₃, 400 MHz) δ 10.16 (br s, 1H), 9.84 (br s, 1H), 8.17 (d, J=7.82 Hz, 1H), 7.46-7.59 (m, 1H), 7.40 (m, 1H), 6.97-7.21 (m, 3H), 5.03 (br s, 1H), 4.06-4.27 (m, 3H), 1.44-1.52 (m, 9H).

### Step C: 2-((2-(3-tert-Butoxycarbonyl)amino)propyl)-4-fluorophenyl)amino)-4-(trifluoro-methyl)benzoic acid 28c

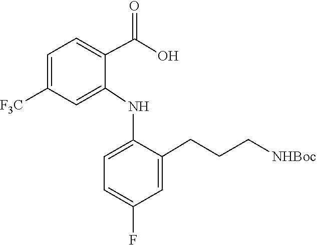 [SMILES: CC(C)(C)OC(=O)NCCCc1cc(F)ccc1Nc1cc(C(F)(F)F)ccc1C(=O)O]

Following the procedure outlined in Example 7, Step C, substituting MeOH with EtOAc and stirring the the reaction mixture at room temperature for 3 h, 2-((2-(3-tert-butoxycarbonyl)amino)propyl)-4-fluorophenyl)amino)-4-(trifluoromethyl)benzoic acid 28c (400 mg, 99% yield) was prepared as a yellow oil, which solidified upon storage. HPLC/MS 1.36 min (A), [M+H]⁺ 457.1. ¹H NMR (CDCl₃, 400 MHz) δ 9.1-9.7 (m, 1H), 8.14 (d, 1H, J=7.8 Hz), 7.2-7.3 (m, 1H), 6.9-7.1 (m, 4H), 4.67 (br s, 1H), 3.0-3.4 (m, 2H), 2.62 (br s, 2H), 2.07 (s, 1H), 1.8-1.8 (m, 1H), 1.7-1.8 (m, 1H), 1.44 (s, 9H).

### Step D: tert-Butyl (3-(2-((2-((2-bromo-6-methoxypyridin-3-yl)carbamoyl-5-(trifluoro-methyl)phenyl)amino)-5-fluorophenyl)propyl)carbamate 28d

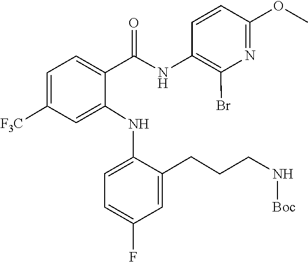 [SMILES: COc1ccc(NC(=O)c2ccc(C(F)(F)F)cc2Nc2ccc(F)cc2CCCNC(=O)OC(C)(C)C)c(Br)n1]

Following the procedure outlined in Example 15, Step F, stirring the reaction mixture at room temperature for 60 h, tert-butyl (3-(2-((2-((2-bromo-6-methoxypyridin-3-yl)carbamoyl-4-(trifluoromethyl)phenyl) amino)-5-fluorophenyl)propyl)carbamate 28d (331 mg, 59% yield) was prepared as a red oil. HPLC/MS 1.50 min (A), [M+H]⁺ 507.8. ¹H NMR (CDCl₃, 400 MHz) δ 9.25 (s, 1H), 8.52 (d, 1H, J=8.8 Hz), 8.2-8.3 (m, 1H), 7.7-7.9 (m, 1H), 7.29 (s, 1H), 6.9-7.1 (m, 4H), 6.81 (d, 1H, J=8.8 Hz), 4.6-4.9 (m, 1H), 3.97 (s, 3H), 3.1-3.3 (m, 2H), 2.6-2.8 (m, 2H), 1.40 (s, 9H).

### Step E: tert-Butyl (3-(2-(3-(2-bromo-6-methoxypyridin-3-yl)-4-oxo-7-(trifluoromethyl)-3,4-dihydro quinazolin-1(2H)-yl)-5-fluorophenyl)propyl)carbamate 28e

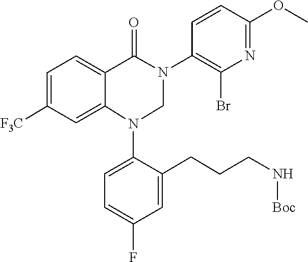 [SMILES: COc1ccc(N2CN(c3ccc(F)cc3CCCNC(=O)OC(C)(C)C)c3cc(C(F)(F)F)ccc3C2=O)c(Br)n1]

Following the procedure outlined in Example 11, Step E, stirring the reaction mixture at 85° C. for 16 h, tert-butyl (3-(2-(3-(2-bromo-6-methoxypyridin-3-yl)-4-oxo-6-(trifluoromethyl)-3,4-dihydroquinazolin-1(2H)-yl)-5-fluorophenyl)propyl)carbamate 28e (140 mg, 42% yield) was prepared as a red oil, in 85% purity. HPLC/MS 1.41 min (A), [M+H]⁺ 598.8.

### Step E: 1-(2-(3-Aminopropyl)-4-fluorophenyl)-3-(2-bromo-6-methoxypyridin-3-yl)-7-(trifluoromethyl)-2,3-dihydroquinazolin-4(11H)-one, hydrochloride salt 28f

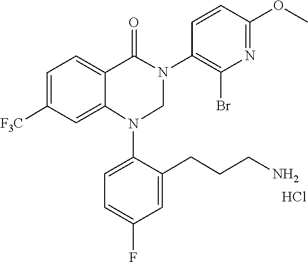 [SMILES: COc1ccc(N2CN(c3ccc(F)cc3CCCN)c3cc(C(F)(F)F)ccc3C2=O)c(Br)n1 ; Cl]

Following the procedure outlined in Example 25, Step F, stirring the reaction mixture at room temperature for 2 h, to which was added an additional 10 equiv. of 4N dioxane/HCl and the reaction mixture continued to stir at room temperature for 48 h, 1-(2-(3-aminopropyl)-4-fluorophenyl)-3-(2-bromo-6-methoxypyridin-3-yl)-7-(trifluoromethyl)-2,3-dihydroquinazolin-4(1H)-one, hydrochloride salt 28f (110 mg, 93% yield) was prepared as a red solid, in 52% purity. HPLC/MS 0.87 min (A), [M+H]⁺ 554.9.

### Step F: 8-Fluoro-15-methoxy-3-(trifluoromethyl)-10,11,12,13-tetrahydro-19H-5,18-methanodibenzo[f,i]pyrido[2,3,-b][1,4,8]triazacyclotridecine-19-one 282

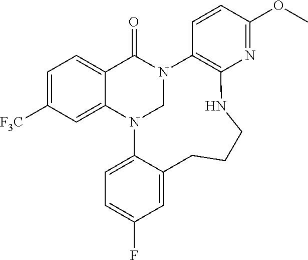 [SMILES: COc1ccc2c(n1)NCCCc1cc(F)ccc1N1CN2C(=O)c2ccc(C(F)(F)F)cc21]

Following the procedure outlined in Example 25, Step G, substituting Pd(OAc)₂ with Pd₂(dba)₃ and stirring the reaction mixture at 90° C. for 2 h, 8-fluoro-15-methoxy-3-(trifluoromethyl)-10,11,12,13-tetrahydro-19H-5,18-methanodibenzo[f,i]pyrido[2,3,-b][1,4,8]triazacyclotridecine-19-one 28g (42 mg, 49% yield) was prepared as a light yellow solid. HPLC/MS 1.27 min (A), [M+H]⁺ 473.1. ¹H NMR (CDCl₃, 400 MHz) δ 8.26 (d, J=7.82 Hz, 1H), 7.47 (d, J=8.31 Hz, 1H), 7.15-7.28 (m, 3H), 7.02-7.13 (m, 1H), 6.57 (s, 1H), 6.14 (d, J=8.31 Hz, 1H), 5.57 (br s, 1H), 5.39 (d, J=10.76 Hz, 1H), 4.31 (d, J=10.76 Hz, 1H), 3.88 (s, 3H), 3.55 (br s, 1H), 3.41 (m, 1H), 2.83-3.01 (m, 1H), 2.74 (m, 1H), 2.26 (m, 1H), 1.51-1.74 (m, 1H).

### Step G: 8-Fluoro-3-(trifluoromethyl)-10,11,12,13-tetrahydro-19H-5,18-methanodibenzo[f,i]pyrido[2,3-b][1,4,8]triazocyclotridecine-15,19(14H)-dione Example 28

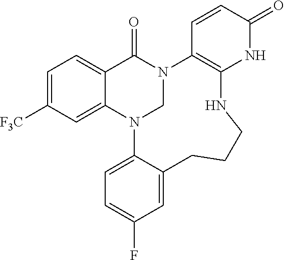 [SMILES: O=C1c2ccc(C(F)(F)F)cc2N2CN1c1ccc(=O)[nH]c1NCCCc1cc(F)ccc12]

Following the procedure outlined in Example 7, Step G, stirring the reaction mixture at 75° C. for 18 h, then 95° C. for 4 h, 8-fluoro-3-(trifluoromethyl)-10,11,12,13-tetrahydro-19H-5,18-methanodibenzo [f,i]pyrido[2,3-b][1,4,8]triazocyclotridecine-15,19(14H)-dione (16.4 mg, 56% yield) was prepared as a white solid. HPLC/MS 0.99 min (A), [M+H]⁺ 459.1. ¹H NMR (CD₃OD, 400 MHz) δ 8.19 (br d, J=7.82 Hz, 1H), 7.53 (br d, J=8.80 Hz, 1H), 7.43 (br s, 1H), 7.09-7.36 (m, 3H), 6.56 (br s, 1H), 5.98 (br d, J=8.80 Hz, 1H), 5.57 (br d, J=10.76 Hz, 1H), 4.52-4.80 (m, 2H), 3.24 (br d, J=6.85 Hz, 1H), 2.73 (br d, J=8.31 Hz, 1H), 2.63 (br s, 1H), 2.15 (br s, 1H), 1.55 (br s, 1H).

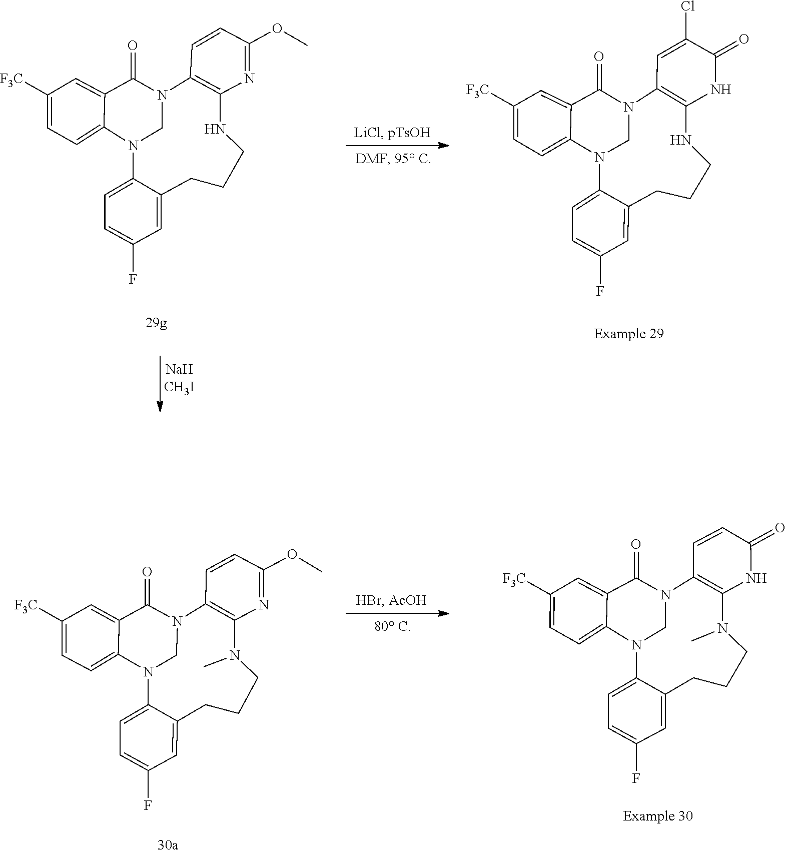 [SMILES: COc1ccc2c(n1)NCCCc1cc(F)ccc1N1CN2C(=O)c2cc(C(F)(F)F)ccc21 ; O=C1c2cc(C(F)(F)F)ccc2N2CN1c1cc(Cl)c(=O)[nH]c1NCCCc1cc(F)ccc12 ; COc1ccc2c(n1)N(C)CCCc1cc(F)ccc1N1CN2C(=O)c2cc(C(F)(F)F)ccc21 ; CN1CCCc2cc(F)ccc2N2CN(C(=O)c3cc(C(F)(F)F)ccc32)c2ccc(=O)[nH]c21]

### Example 29

### 16-Chloro-8-fluoro-2-(trifluoromethyl)-10,11,12,13-tetrahydro-19H-5,18-methanodibenzo[f,i]pyrido[2,3,-b][1,4,8]triazacyclotridecine-15,19(14H)-dione

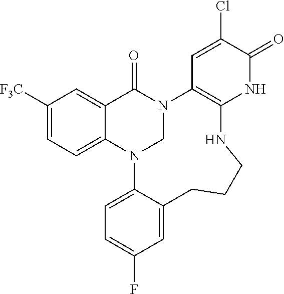 [SMILES: O=C1c2cc(C(F)(F)F)ccc2N2CN1c1cc(Cl)c(=O)[nH]c1NCCCc1cc(F)ccc12]

### Step A: Methyl 2-((2-(3-((tert-butoxycarbonyl)amino)prop-1-yn-1-yl)-4-fluorophenyl)-amino)-5-(trifluoromethyl)benzoate 29a

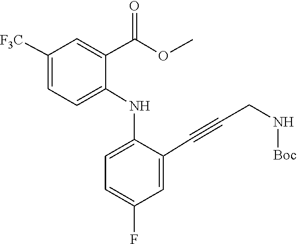 [SMILES: COC(=O)c1cc(C(F)(F)F)ccc1Nc1ccc(F)cc1C#CCNC(=O)OC(C)(C)C]

Following the procedure outlined in Example 7, Step A, substituting Int-3a with Int-3f, stirring the reaction mixture at 95° C. for 18 h, methyl 2-((2-(3-((tert-butoxycarbonyl)(6-methoxy-3-nitropyridin-2-yl)amino)prop-1-yn-1-yl)-4-fluorophenyl)amino)-5-(trifluoromethyl)benzoate 29a (500 mg, 30% yield) was prepared as a light yellow oil. HPLC/MS 1.49 min (A), [M+H]⁺ 467.1. ¹H NMR (CDCl₃, 400 MHz) δ 10.1-10.5 (m, 1H), 8.29 (d, 1H, J=1.5 Hz), 7.5-7.6 (m, 1H), 7.4-7.5 (m, 1H), 7.37 (d, 1H, J=8.8 Hz), 7.18 (dd, 1H, J=2.9, 8.8 Hz), 7.0-7.1 (m, 1H), 5.2-5.5 (m, 1H), 4.1-4.3 (m, 2H), 4.01 (s, 3H), 1.46 (s, 9H).

### Step B: 2-((2-(3-tert-Butoxycarbonyl)amino)prop-1-yn-1-yl)-4-fluorophenyl)amino)-5-(trifluoromethyl) benzoic acid 29b

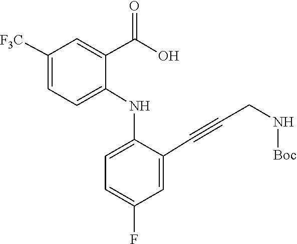 [SMILES: CC(C)(C)OC(=O)NCC#Cc1cc(F)ccc1Nc1ccc(C(F)(F)F)cc1C(=O)O]

Following the procedure outlined in Example 7, Step B, stirring the reaction mixture in THF/MeOH/H₂O at room temperature for 72 h, 2-((2-(3-tert-butoxycarbonyl)(6-methoxy-3-nitropyridin-2-yl)amino)prop-1-yn-1-yl)-4-fluorophenyl)amino)-5-(trifluoromethyl)benzoic acid 29b (480 mg, 98% yield) was prepared as a yellow solid. HPLC/MS 1.38 min (A), [M+H]⁺ 453.0. ¹H NMR (CDCl₃, 400 MHz) δ 10.5-11.1 (m, 1H), 9.8-10.4 (m, 1H), 8.34 (d, 1H, J=1.0 Hz), 7.5-7.6 (m, 1H), 7.4-7.5 (m, 1H), 7.2-7.3 (m, 1H), 7.1-7.2 (m, 1H), 7.0-7.1 (m, 1H), 4.9-6.6 (m, 1H), 4.15 (br d, 3H, J=7.3 Hz), 1.45 (s, 9H).

### Step C: 2-((2-(3-tert-Butoxycarbonyl)amino)propyl)-4-fluorophenyl)amino)-5-(trifluoro-methyl)benzoic acid 29c

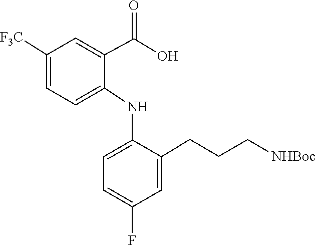 [SMILES: CC(C)(C)OC(=O)NCCCc1cc(F)ccc1Nc1ccc(C(F)(F)F)cc1C(=O)O]

Following the procedure outlined in Example 27, Step C, substituting EtOH with EtOAc and stirring the reaction mixture overnight, 2-((2-(3-tert-butoxycarbonyl)amino)propyl)-4-fluorophenyl)amino)-5-(trifluoromethyl)benzoic acid 29c (500 mg, 98% yield) was prepared as a yellow oil, which solidified upon storage. HPLC/MS 1.35 min (A), [M+H]⁺ 457.1. ¹H NMR (CDCl₃, 400 MHz) δ9.3-9.8 (m, 1H), 8.32 (d, 1H, J=1.5 Hz), 7.4-7.6 (m, 1H), 7.2-7.3 (m, 1H), 6.98 (br d, 2H, J=2.9 Hz), 6.7-6.8 (m, 1H), 4.5-6.4 (m, 1H), 3.0-3.3 (m, 2H), 2.5-2.7 (m, 2H), 1.7-1.8 (m, 2H), 1.42 (s, 9H).

### Step D: tert-Butyl (3-(2-((2-((2-bromo-6-methoxypyridin-3-yl)carbamoyl-5-(trifluoro-methyl)phenyl)amino)-5-fluorophenyl)propyl)carbamate 29d

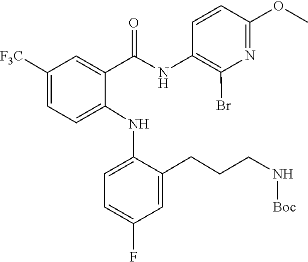 [SMILES: COc1ccc(NC(=O)c2cc(C(F)(F)F)ccc2Nc2ccc(F)cc2CCCNC(=O)OC(C)(C)C)c(Br)n1]

Following the procedure outlined in Example 15, Step F, stirring the reaction mixture at room temperature for 60 h, tert-butyl (3-(2-((2-((2-bromo-6-methoxypyridin-3-yl)carbamoyl-5-(trifluoromethyl)phenyl) amino)-5-fluorophenyl)propyl)carbamate 29d (1.20 g, 65% yield) was prepared as a light pink solid. HPLC/MS 1.50 min (A), [M+H]⁺ 643.0. ¹H NMR (CDCl₃, 400 MHz) δ 9.3-9.5 (m, 1H), 8.47 (d, 1H, J=8.3 Hz), 8.1-8.2 (m, 1H), 7.8-8.0 (m, 1H), 7.4-7.6 (m, 1H), 7.2-7.3 (m, 1H), 7.0-7.1 (m, 1H), 6.9-7.0 (m, 1H), 6.8-6.9 (m, 2H), 4.5-4.9 (m, 1H), 3.98 (s, 3H), 3.89 (s, 1H), 3.1-3.3 (m, 2H), 2.5-2.8 (m, 2H), 1.7-1.9 (m, 2H), 1.40 (s, 9H).

### Step E: tert-Butyl (3-(2-(3-(2-bromo-6-methoxypyridin-3-yl)-4-oxo-6-(trifluoromethyl)-3,4-dihydro quinazolin-1(2H)-yl)-5-fluorophenyl)propyl)carbamate 29e

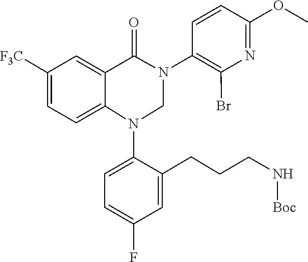 [SMILES: COc1ccc(N2CN(c3ccc(F)cc3CCCNC(=O)OC(C)(C)C)c3ccc(C(F)(F)F)cc3C2=O)c(Br)n1]

Following the procedure outlined in Example 15, Step G, stirring the reaction mixture at 85° C. for 16 h, tert-butyl (3-(2-(3-(2-bromo-6-methoxypyridin-3-yl)-4-oxo-6-(trifluoromethyl)-3,4-dihydroquinazolin-1(2H)-yl)-5-fluorophenyl)propyl) carbamate 29e (820 mg, 52% yield) was prepared as a red solid, in 80% purity. HPLC/MS 1.41 min (A), [M+H]⁺ 598.8.

### Step F: 1-(2-(3-Aminopropyl)-4-fluorophenyl)-3-(2-bromo-6-methoxypyridin-3-yl)-6-(trifluoromethyl)-2,3-dihydroquinazolin-4(11H)-one, hydrochloride salt 29f

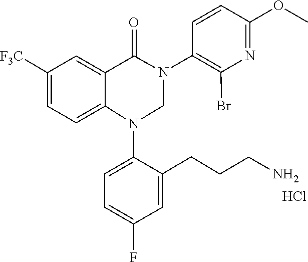 [SMILES: COc1ccc(N2CN(c3ccc(F)cc3CCCN)c3ccc(C(F)(F)F)cc3C2=O)c(Br)n1 ; Cl]

Following the procedure outlined in Example 28, Step F, stirring the reaction mixture at 50° C. for 30 min, 1-(2-(3-aminopropyl)-4-fluorophenyl)-3-(2-bromo-6-methoxypyridin-3-yl)-6-(trifluoromethyl)-2,3-dihydroquinazolin-4(1H)-one, hydrochloride salt 29f (760 mg, 97% yield) was prepared as a red solid, in 81% purity. HPLC/MS 0.88 min (A), [M+H]⁺ 554.9.

### Step G: 8-Fluoro-15-methoxy-2-(trifluoromethyl)-10,11,12,13-tetrahydro-19H-5,18-methanodibenzo[f,i]pyrido[2,3,-b][1,4,8]triazacyclotridecine-19-one 29g

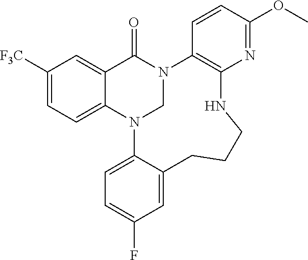 [SMILES: COc1ccc2c(n1)NCCCc1cc(F)ccc1N1CN2C(=O)c2cc(C(F)(F)F)ccc21]

Following the procedure outlined in Example 28, Step G, stirring the reaction mixture at 90° C. for 2 h, 8-fluoro-15-methoxy-2-(trifluoromethyl)-10,11,12,13-tetrahydro-19H-5,18-methanodibenzo[f,i]pyrido [2,3,-b][1,4,8]triazacyclotridecine-19-one 29g (160 mg) was prepared as a light yellow solid. HPLC/MS 1.27 min (A), [M+H]⁺ 473.1. ¹H NMR (CDCl₃, 400 MHz) δ 8.42 (d, 1H, J=2.4 Hz), 7.5-7.6 (m, 1H), 7.47 (d, 1H, J=8.3 Hz), 7.1-7.2 (m, 2H), 7.0-7.1 (m, 1H), 6.4-6.5 (m, 1H), 6.14 (d, 1H, J=8.3 Hz), 5.4-5.5 (m, 1H), 5.40 (d, 1H, J=11.2 Hz), 4.30 (s, 1H), 3.87 (s, 3H), 3.5-3.6 (m, 1H), 3.3-3.5 (m, 1H), 2.8-2.9 (m, 1H), 2.7-2.8 (m, 1H), 2.2-2.3 (m, 1H), 1.5-1.8 (m, 2H).

### Step H: 16-Chloro-8-fluoro-2-(trifluoromethyl)-10,11,12,13-tetrahydro-19H-5,18-methanodibenzo[f,i]pyrido[2,3,-b][1,4,8]triazacyclotridecine-15,19(14H)-dione

### Example 29

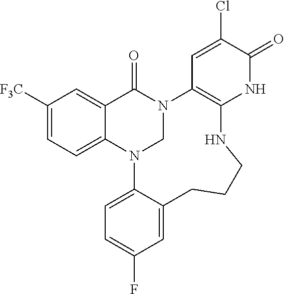 [SMILES: O=C1c2cc(C(F)(F)F)ccc2N2CN1c1cc(Cl)c(=O)[nH]c1NCCCc1cc(F)ccc12]

Following the procedure outlined in Example 28, Step H, stirring the reaction mixture at 75° C. for 18 h, then 95° C. for 4 h, 16-chloro-8-fluoro-2-(trifluoromethyl)-10,11,12,13-tetrahydro-19H-5,18-methano dibenzo[f,i]pyrido[2,3,-b][1,4,8]triazacyclotridecine-15,19(14H)-dione (10 mg, 11% yield) was prepared as an off-white solid. HPLC/MS 1.08 min (A), [M+H]⁺ 493.1. ¹H NMR (CD₃OD, 400 MHz) δ 8.28 (s, 1H), 7.62 (m, 1H), 7.42 (m, 1H), 7.25 (m, 1H), 7.15 (m, 1H), 6.52 (d, J=8.31 Hz, 1H), 5.56 (d, J=11.25 Hz, 1H), 4.96 (br d, J=8.31 Hz, 1H), 4.68 (br d, J=11.25 Hz, 2H), 3.36-3.62 (m, 2H), 3.12-3.29 (m, 1H), 2.67-2.95 (m, 1H), 2.58 (m, 1H), 1.99-2.19 (m, 1H), 1.54 (br s, 1H).

### Example 30

### 8-Fluoro-13-methyl-2-(trifluoromethyl)-10,11,12,13-tetrahydro-19H-5,18-methanodibenzo[f,i]pyrido[2,3,-b][1,4,8]triazacyclotridecine-15,19(14H)-dione

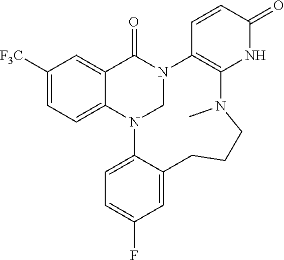 [SMILES: CN1CCCc2cc(F)ccc2N2CN(C(=O)c3cc(C(F)(F)F)ccc32)c2ccc(=O)[nH]c21]

### Step A: 8-Fluoro-15-methoxy-13-methyl-2-(trifluoromethyl)-10,11,12,13-tetrahydro-19H-5,18-methanodibenzo[f,i]pyrido[2,3,-b][1,4,8]triazacyclotridecine-19-one 30a

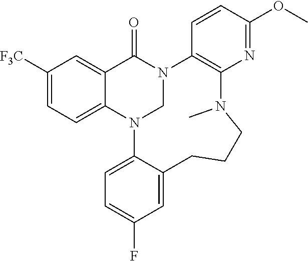 [SMILES: COc1ccc2c(n1)N(C)CCCc1cc(F)ccc1N1CN2C(=O)c2cc(C(F)(F)F)ccc21]

To 8-fluoro-15-methoxy-2-(trifluoromethyl)-10,11,12,13-tetrahydro-19H-5,18-methanodibenzo[f,i]pyrido[2,3-b][1,4,8]triazacyclotridecin-19-one (55 mg, 0.116 mmol) in DMF (2 mL) at room temperature was added 60% NaH (93 mg, 2.33 mmol) and the reaction mixture was allowed to stir for 20 min. Methyl iodide (10.92 μl, 0.175 mmol) was added and the reaction was allowed to stir at room temperature for 48 h. The reaction was partitioned between 50 mL of EtOAc and 25 mL of water, the layers separated and the aqueous layer extracted with EtOAc (20 mL). The combined organic layers were washed with brine, dried over Na₂SO₄, and concentrated to dryness under reduced pressure to afford 8-fluoro-15-methoxy-13-methyl-2-(trifluoromethyl)-10,11,12,13-tetrahydro-19H-5,18-methanodibenzo[f,i]pyrido[2,3-b][1,4,8]triazacyclotridecin-19-one 30a (45 mg, 79% yield) as a pale yellow solid. HPLC/MS 1.27 min (A), [M+H]⁺ 487.1.

### Step B: 8-Fluoro-13-methyl-2-(trifluoromethyl)-10,11,12,13-tetrahydro-19H-5,18-methanodibenzo[f,i]pyrido[2,3,-b][1,4,8]triazacyclotridecine-15,19(14H)-dione

### Example 30

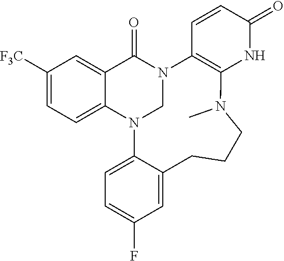 [SMILES: CN1CCCc2cc(F)ccc2N2CN(C(=O)c3cc(C(F)(F)F)ccc32)c2ccc(=O)[nH]c21]

To 8-fluoro-15-methoxy-13-methyl-2-(trifluoromethyl)-10,11,12,13-tetrahydro-19H-5,18-methano dibenzo[f,i]pyrido[2,3-b][1,4,8]triazacyclotridecin-19-one (45 mg, 0.093 mmol) in AcOH (5 mL) at room temperature was added 33% HBr/AcOH (0.152 mL, 0.925 mmol) and the reaction was allowed to stir at 80° C. overnight. Additional 33% HBr/AcOH (0.152 mL, 0.925 mmol) was added and the reaction mixture was stirred for 2 h. The reaction was concentrated under vacuum, dissolved in 2 mL of DMSO and purified on a 30g C-18 column eluting with 5-95% ACN/water (0.10% TFA). Product fractions were combined and evaporated under reduced pressure to afford 8-fluoro-13-methyl-2-(trifluoromethyl)-10,11,12,13-tetrahydro-19H-5,18-methanodibenzo[f,i]pyrido[2,3,-b][1,4,8]triazacyclotridecine-15,19(14H)-dione (12 mg, 28% yield) as a brown solid, in 85% purity. HPLC/MS 1.05 min (A), [M+H]⁺ 473.1.

### Example 31

### 8-Fluoro-2-(trifluoromethoxy)-10,11,12,13-tetrahydro-19H-5,18-methanodibenzo[f,i]pyrido[2,3,-b][1,4,8]triazacyclotridecine-15,19(14H)-dione

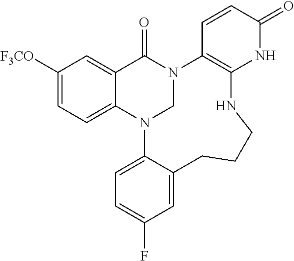 [SMILES: O=C1c2cc(OC(F)(F)F)ccc2N2CN1c1ccc(=O)[nH]c1NCCCc1cc(F)ccc12]

### Step A: Methyl 2-((2-(3-((tert-butoxycarbonyl)(6-methoxy-3-nitropyridin-2-yl)amino)-prop-1-yn-1-yl)-4-fluorophenyl)amino)-5-(trifluoromethoxy)benzoate 31a

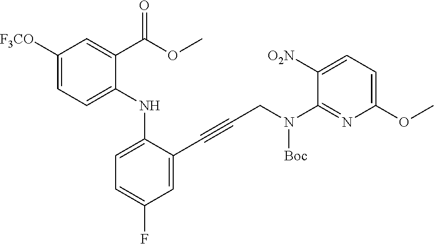 [SMILES: COC(=O)c1cc(OC(F)(F)F)ccc1Nc1ccc(F)cc1C#CCN(C(=O)OC(C)(C)C)c1nc(OC)ccc1[N+](=O)[O-]]

Following the procedure outlined in Example 27, Step A, substituting Int-2 with Int-2d, Pd(OAc)₂ with Pd₂(dba)₃, Xantphos with BINAP, 1,4-dioxane with toluene, and stirring the reaction mixture at 90° C. for 17 h, methyl 2-((2-(3-((tert-butoxycarbonyl)(6-methoxy-3-nitropyridin-2-yl)amino)prop-1-yn-1-yl)-4-fluorophenyl)amino)-5-(trifluoromethoxy)benzoate 31a (4.56 g, 27% yield) was prepared as a white foam. HPLC/MS 1.61 min (A), [M+H]⁺ 535.3. ¹H NMR (DMSO-d₆, 400 MHz) δ 9.2-9.5 (m, 1H), 8.28 (d, 1H, J=8.8 Hz), 7.7-7.7 (m, 1H), 7.4-7.5 (m, 2H), 7.3-7.4 (m, 1H), 7.2-7.3 (m, 1H), 7.12 (d, 1H, J=9.3 Hz), 6.78 (d, 1H, J=8.8 Hz), 4.90 (s, 2H), 3.85 (s, 3H), 3.73 (s, 3H), 1.1-1.5 (m, 11H).

### Step B: 2-((2-(3-tert-Butoxycarbonyl)(6-methoxy-3-nitropyridin-2-yl)amino)prop-1-yn-1-yl)-4-fluorophenyl)amino)-5-(trifluoromethoxy)benzoic acid 31b

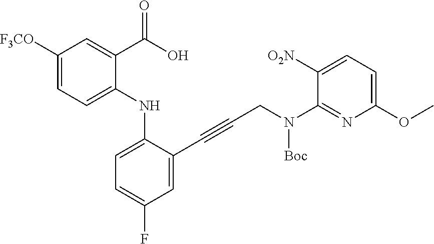 [SMILES: COc1ccc([N+](=O)[O-])c(N(CC#Cc2cc(F)ccc2Nc2ccc(OC(F)(F)F)cc2C(=O)O)C(=O)OC(C)(C)C)n1]

Following the procedure outlined in Example 27, Step B, stirring the reaction mixture at 50° C. for 24 h, 2-((2-(3-tert-butoxycarbonyl)(6-methoxy-3-nitropyridin-2-yl)amino)prop-1-yn-1-yl)-4-fluorophenyl) amino)-5-(trifluoromethoxy)benzoic acid 31b (1.08 g, 97% yield) was prepared as a yellow foam. HPLC/MS 1.50 min (A), [M+H]⁺ 621.3.

### Step C: 2-((2-(3-((3-Amino-6-methoxypyridin-2-yl)(tert-butoxycarbonyl)amino)propyl)-4-fluorophenyl) amino)-5-(trifluoromethoxy)benzoic acid 31c

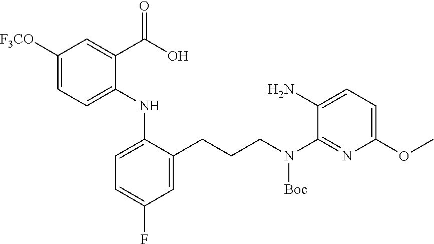 [SMILES: COc1ccc(N)c(N(CCCc2cc(F)ccc2Nc2ccc(OC(F)(F)F)cc2C(=O)O)C(=O)OC(C)(C)C)n1]

Following the procedure outlined in Example 27, Step C, substituting EtOH with EtOAc and stirring the reaction mixture at room temperature for 108 h, 2-((2-(3-((3-amino-6-methoxypyridin-2-yl)(tert-butoxy carbonyl)amino)propyl)-4-fluorophenyl)amino)-5-(trifluoromethoxy)benzoic acid 31c (512 mg, 41% yield) was prepared as a beige foam. HPLC/MS 1.35 min (A), [M+H]⁺ 595.3. ¹H NMR (DMSO-d₆, 400 MHz) δ 9.3-9.5 (m, 1H), 7.7-7.8 (m, 1H), 7.3-7.4 (m, 3H), 7.1-7.2 (m, 1H), 7.0-7.1 (m, 2H), 6.61 (d, 1H, J=9.3 Hz), 6.5-6.6 (m, 1H), 3.62 (s, 3H), 3.4-3.6 (m, 2H), 3.3-3.4 (m, 2H), 2.5-2.6 (m, 2H), 1.6-1.8 (m, 2H), 1.30 (s, 9H).

### Step D: tert Butyl 8-fluoro-15-methoxy-19-oxo-2-(trifluoromethoxy)-5,10,11,12,18,19-hexahydro-13H-dibenzo[f,i]pyrido[2,3,-b][1,4,8]triazacyclotridecine-13-carboxylate 31d

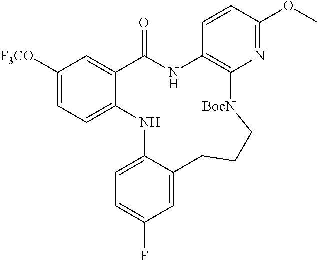 [SMILES: COc1ccc2c(n1)N(C(=O)OC(C)(C)C)CCCc1cc(F)ccc1Nc1ccc(OC(F)(F)F)cc1C(=O)N2]

Following the procedure outlined in Example 27, Step D, stirring the reaction mixture at room temperature for 1.45 h, tert butyl 8-fluoro-15-methoxy-19-oxo-2-(trifluoromethoxy)-5,10,11,12,18,19-hexahydro-13H-dibenzo[f,i]pyrido[2,3,-b][1,4,8]triazacyclo tridecine-13-carboxylate 31d (293 mg, 61% yield) was prepared as a light yellow foam. HPLC/MS 1.53 min (A), [M+H]⁺ 577.2. ¹H NMR (DMSO-d₆, 400 MHz) δ 9.2-9.9 (m, 1H), 8.2-8.9 (m, 1H), 7.96 (br s, 1H), 7.6-7.8 (m, 1H), 7.36 (br d, 1H, J=8.3 Hz), 7.24 (br d, 1H, J=9.3 Hz), 7.0-7.2 (m, 2H), 6.87 (d, 1H, J=8.8 Hz), 6.68 (br d, 1H, J=8.8 Hz), 3.9-4.1 (m, 1H), 3.82 (s, 3H), 3.5-3.8 (m, 1H), 2.7-2.9 (m, 1H), 1.6-1.9 (m, 1H), 1.4-1.6 (m, 1H), 0.9-1.3 (m, 9H).

### Step E: tert-Butyl 8-fluoro-15-methoxy-19-oxo-2-(trifluoromethoxy)-11,12-dihydro-19H-5,18-methano dibenzo[f,i]pyrido[2,3,-b][1,4,8]triazacyclotridecine-13(10H)-carboxylate

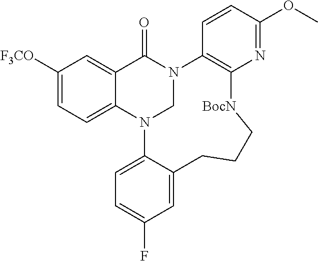 [SMILES: COc1ccc2c(n1)N(C(=O)OC(C)(C)C)CCCc1cc(F)ccc1N1CN2C(=O)c2cc(OC(F)(F)F)ccc21]

Following the procedure outlined in Example 27, Step E, stirring the reaction mixture at 90° C. for 23 h, tert butyl 8-fluoro-15-methoxy-19-oxo-2-(trifluoromethoxy)-11,12-dihydro-19H-5,18-methanodibenzo [f,i]pyrido[2,3,b][1,4,8]triazacyclo tridecine-13(10H)-carboxylate 31e (250 mg, 91% yield) was prepared as a white foam. HPLC/MS 1.44 min (A), [M+H]⁺ 617.0. ¹H NMR (DMSO-d₆, 400 MHz) δ 7.88 (d, 1H, J=8.3 Hz), 7.79 (d, 1H, J=2.0 Hz), 7.4-7.5 (m, 1H), 7.3-7.4 (m, 2H), 7.17 (s, 1H), 6.90 (d, 1H, J=8.3 Hz), 6.4-6.6 (m, 1H), 5.32 (s, 1H), 4.3-4.6 (m, 1H), 3.85 (s, 3H), 3.6-3.8 (m, 1H), 3.0-3.3 (m, 1H), 2.5-2.6 (m, 1H), 1.8-2.0 (m, 1H), 1.3-1.6 (m, 1H), 1.09 (s, 9H).

### Step F: 8-Fluoro-2-(trifluoromethoxy)-10,11,12,13-tetrahydro-19H-5,18-methanodibenzo[f,i]pyrido[2,3,-b][1,4,8]triazacyclotridecine-15,19(14H)-dione

### Example 31

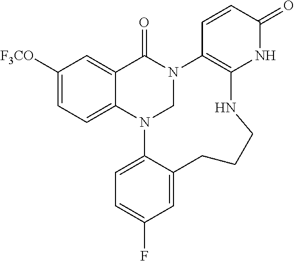 [SMILES: O=C1c2cc(OC(F)(F)F)ccc2N2CN1c1ccc(=O)[nH]c1NCCCc1cc(F)ccc12]

Following the procedure outlined in Example 27, Step F, stirring the reaction mixture at 90° C. for 48 h, 8-fluoro-2-(trifluoromethoxy)-10,11,12,13-tetrahydro-19H-5,18-methanodibenzo[f,i]pyrido[2,3,-b][1,4,8]triazacyclotridecine-15,19(14H)-dione (73 mg, 44% yield) was prepared as a white solid. HPLC/MS 1.02 min (A), [M+H]⁺ 475.1. ¹H NMR (DMSO-d₆, 400 MHz) δ 10.4-11.1 (m, 1H), 7.6-7.9 (m, 1H), 7.45 (dd, 1H, J=5.4, 8.8 Hz), 7.3-7.4 (m, 3H), 7.21 (dt, 1H, J=3.2, 8.4 Hz), 6.34 (d, 1H, J=9.3 Hz), 5.6-5.8 (m, 2H), 5.47 (d, 1H, J=11.2 Hz), 4.44 (d, 1H, J=10.8 Hz), 3.04 (br d, 1H, J=5.4 Hz), 2.69 (br dd, 1H, J=3.9, 8.8 Hz), 2.54 (br s, 1H), 1.9-2.1 (m, 1H), 1.41 (br dd, 1H, J=4.4, 7.8 Hz).

### Example 32

### 2,8-Difluoro-3-(trifluoromethyl)-10,11,12,13-tetrahydro-19H-5,18-methanodibenzo[f,i]pyrido[2,3,-b][1,4,8]triazacyclotridecine-15,19(14H)-dione

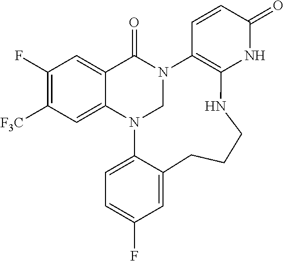 [SMILES: O=C1c2cc(F)c(C(F)(F)F)cc2N2CN1c1ccc(=O)[nH]c1NCCCc1cc(F)ccc12]

### Step A: Methyl 2-((2-(3-((tert-butoxycarbonyl)(6-methoxy-3-nitropyridin-2-yl amino)-prop-1-yn-1-yl)-4-fluorophenyl amino)-5-fluoro-4-(trifluoromethyl)benzoate 32a

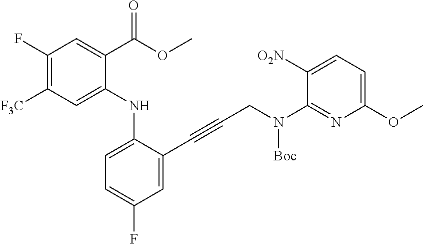 [SMILES: COC(=O)c1cc(F)c(C(F)(F)F)cc1Nc1ccc(F)cc1C#CCN(C(=O)OC(C)(C)C)c1nc(OC)ccc1[N+](=O)[O-]]

Following the procedure outlined in Example 27, Step A, substituting Int-2 with Int-2e, stirring the reaction mixture at 80° C. for 18 h, methyl 2-((2-(3-((tert-butoxycarbonyl)(6-methoxy-3-nitropyridin-2-yl)amino)prop-1-yn-1-yl)-4-fluorophenyl) amino)-5-fluoro-4-(trifluoromethyl)benzoate 32a (1.86 g, 81% yield) was prepared as a yellow oil. HPLC/MS 1.67 min (B), [M+H]⁺ 637.1. ¹H NMR (CDCl₃, 400 MHz) δ 9.36 (br s, 1H), 8.18 (d, J=9.3 Hz, 1H), 7.73 (d, J=10.8 Hz, 1H), 7.21-7.27 (m, 2H), 7.15 (dd, J=8.3, 2.9 Hz, 1H), 6.97-7.10 (m, 1H), 4.95 (s, 2H), 3.79-4.00 (m, 6H), 1.28-1.43 (m, 9H).

### Step B: Methyl 2-((2-(3-((3-amino-6-methoxypyridin-2-yl)(tert-butoxycarbonyl)amino)-propyl)-4-fluorophenyl)amino)-5-fluoro-4-(trifluoromethyl)benzoate 32b

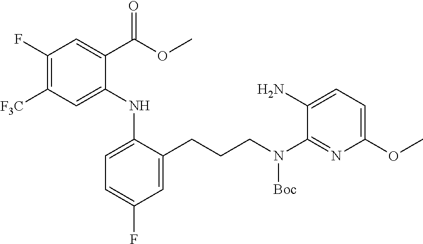 [SMILES: COC(=O)c1cc(F)c(C(F)(F)F)cc1Nc1ccc(F)cc1CCCN(C(=O)OC(C)(C)C)c1nc(OC)ccc1N]

Following the procedure outlined in Example 27, Step C, substituting EtOH with MeOH/EtOAc and stirring the reaction mixture at room temperature for 16 h, methyl 2-((2-(3-((3-amino-6-methoxypyridin-2-yl)(tert-butoxycarbonyl)amino)propyl)-4-fluorophenyl)amino)-5-fluoro-4-(trifluoromethyl)benzoate 32b (1.59 g, 87% yield) was prepared as a yellow foam. HPLC/MS 1.57 min (B), [M+H]⁺ 611.2. ¹H NMR (CDCl₃, 400 MHz) δ 9.04 (s, 1H), 7.76 (d, J=11.3 Hz, 1H), 7.16 (dd, J=8.8, 5.4 Hz, 1H), 6.92-7.05 (m, 3H), 6.79 (d, J=5.9 Hz, 1H), 6.50 (d, J=8.3 Hz, 1H), 3.95 (s, 3H), 3.74-3.82 (m, 5H), 2.56-2.63 (m, 2H), 1.86 (br d, J=6.9 Hz, 2H), 1.42-1.50 (m, 9H).

### Step C: 2-((2-(3-((3-Amino-6-methoxypyridin-2-yl)(tert-butoxycarbonyl)amino)propyl)-4-fluorophenyl) amino)-5-fluoro-4-(trifluoromethyl)benzoic acid 32c

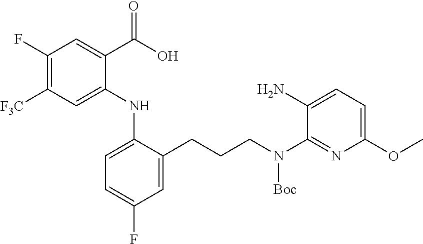 [SMILES: COc1ccc(N)c(N(CCCc2cc(F)ccc2Nc2cc(C(F)(F)F)c(F)cc2C(=O)O)C(=O)OC(C)(C)C)n1]

Following the procedure outlined in Example 27, Step B, stirring the reaction mixture in THF/MeOH/H₂O at room temperature for 2 h, 2-((2-(3-((3-amino-6-methoxypyridin-2-yl)(tert-butoxycarbonyl)amino) propyl)-4-fluorophenyl)amino)-5-fluoro-4-(trifluoromethyl)benzoic acid 32c (1.88 g, 100% yield) was prepared as a brown solid. HPLC/MS 1.04 min (B), [M+H]⁺ 597.1. ¹H NMR (CDCl₃, 400 MHz) δ 8.01 (br s, 1H), 7.55 (d, J=10.8 Hz, 1H), 7.06 (dd, J=8.6, 5.1 Hz, 1H), 6.92 (dd, J=9.3, 2.9 Hz, 1H), 6.77-6.85 (m, 2H), 6.44 (d, J=8.8 Hz, 1H), 3.95-4.03 (m, 3H), 3.65 (s, 2H), 2.51-2.59 (m, 2H), 1.78-1.87 (m, 2H), 1.41 (s, 9H).

### Step D: tert Butyl 2,8-difluoro-15-methoxy-19-oxo-3-(trifluoromethyl)-5,10,11,12,18,19-hexahydro-13H-dibenzo[f,i]pyrido[2,3,-b][1,4,8]triazacyclotridecine-13-carboxylate 32d

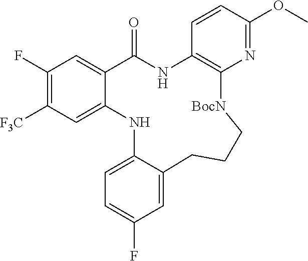 [SMILES: COc1ccc2c(n1)N(C(=O)OC(C)(C)C)CCCc1cc(F)ccc1Nc1cc(C(F)(F)F)c(F)cc1C(=O)N2]

Following the procedure outlined in Example 27, Step D, stirring the reaction mixture in MeCN at room temperature for 16 h, tert butyl 2,8-difluoro-15-methoxy-19-oxo-3-(trifluoromethyl)-5,10,11,12,18,19-hexahydro-13H-dibenzo[f,i]pyrido[2,3,-b][1,4,8]triazacyclotridecine-13-carboxylate 32d (792 mg, 50% yield) was prepared as a yellow foam. HPLC/MS 1.51 min (B), [M+H]⁺ 579.0. ¹H NMR (CDCl₃, 400 MHz) δ 8.34 (br s, 1H), 7.79 (br s, 1H), 7.35-7.57 (m, 2H), 7.22-7.35 (m, 1H), 6.90-7.09 (m, 2H), 6.69-6.82 (m, 2H), 4.14-4.34 (m, 1H), 3.94-4.12 (m, 1H), 3.92 (s, 3H), 2.57 (br s, 1H), 2.30 (br s, 1H), 1.48-1.68 (m, 2H), 1.29 (s, 9H).

### Step E: tert-Butyl 2,8-difluoro-15-methoxy-19-oxo-2-(trifluoromethyl)-11,12-dihydro-19H-5,18-methano dibenzo[f,i]pyrido[2,3,-b][1,4,8]triazacyclotridecine-13(10H)-carboxylate 32e

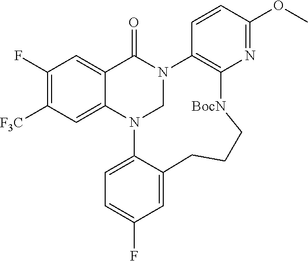 [SMILES: COc1ccc2c(n1)N(C(=O)OC(C)(C)C)CCCc1cc(F)ccc1N1CN2C(=O)c2cc(F)c(C(F)(F)F)cc21]

Following the procedure outlined in Example 27, Step E, stirring the reaction mixture at 90° C. for 24 h, tert-butyl 2,8-difluoro-15-methoxy-19-oxo-2-(trifluoromethyl)-11,12-dihydro-19H-5,18-methanodibenzo [f,i]pyrido[2,3,-b][1,4,8]triazacyclotridecine-13(10H)-carboxylate 32e (664 mg 82% yield) was prepared as a yellow oil. HPLC/MS 1.50 min (B), [M+H]⁺ 591.1. ¹H NMR (CDCl₃, 400 MHz) δ 7.96 (d, J=10.3 Hz, 1H), 7.62-7.83 (m, 1H), 7.22-7.34 (m, 1H), 7.18 (dd, J=9.3, 2.9 Hz, 1H), 7.01 (td, J=8.3, 2.9 Hz, 1H), 6.75 (d, J=8.8 Hz, 2H), 5.23 (br d, J=10.3 Hz, 1H), 3.80-4.01 (m, 4H), 1.87-2.12 (m, 1H), 1.15-1.36 (m, 13H).

### Step F: 2,8-Difluoro-3-(trifluoromethyl)-10,11,12,13-tetrahydro-19H-5,18-methanodibenzo[f,i]pyrido[2,3,-b][1,4,8]triazacyclotridecine-15,19(14H)-dione

### Example 32

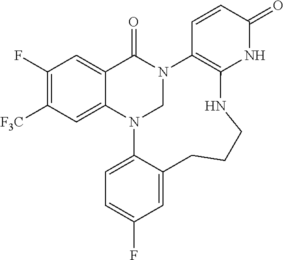 [SMILES: O=C1c2cc(F)c(C(F)(F)F)cc2N2CN1c1ccc(=O)[nH]c1NCCCc1cc(F)ccc12]

Following the procedure outlined in Example 27, Step F, stirring the reaction mixture at 90° C. for 16 h, to which was additional 5N HCl in IPA (90 equiv) was added and heating continued at 90° C. for another 24 h, 2,8-difluoro-3-(trifluoromethyl)-10,11,12,13-tetrahydro-19H-5,18-methanodibenzo[f,i]pyrido[2,3,-b][1,4,8]triazacyclotridecine-15,19(14H)-dione (117 mg, 22% yield) was prepared as an off-white solid. HPLC/MS 1.05 min (B), [M+H]⁺ 477.1. ¹H NMR (DMSO-d₆, 400 MHz) δ 7.88 (d, J=10.3 Hz, 1H), 7.49 (dd, J=8.8, 5.4 Hz, 1H), 7.27-7.38 (m, 2H), 7.22 (td, J=8.4, 3.2 Hz, 1H), 6.41 (d, J=5.4 Hz, 1H), 5.70-5.82 (m, 2H), 5.46 (d, J=11.3 Hz, 1H), 4.47 (br d, J=11.3 Hz, 1H), 3.35-3.46 (m, 1H), 2.91-3.02 (m, 1H), 2.69-2.79 (m, 1H), 2.53-2.63 (m, 1H), 1.92-2.01 (m, 1H), 1.36 (br d, J=5.9 Hz, 1H).

### Example 33

### 1,8-Difluoro-2-(trifluoromethyl)-10,11,12,13-tetrahydro-19H-5,18-methanodibenzo[f,i]pyrido[2,3,-b][1,4,8]triazacyclotridecine-15,19(14H)-dione

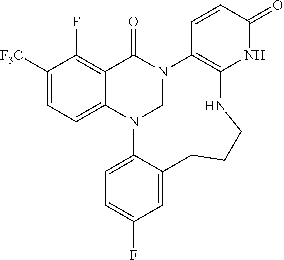 [SMILES: O=C1c2c(ccc(C(F)(F)F)c2F)N2CN1c1ccc(=O)[nH]c1NCCCc1cc(F)ccc12]

### Step A: Methyl 6-((2-(3-((tert-butoxycarbonyl)(6-methoxy-3-nitropyridin-2-yl)amino)-prop-1-yn-1-yl)-4-fluorophenyl)amino)-2-fluoro-3-(trifluoromethyl)benzoate 33a

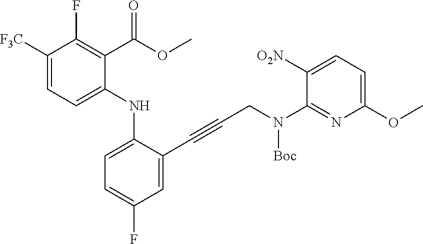 [SMILES: COC(=O)c1c(Nc2ccc(F)cc2C#CCN(C(=O)OC(C)(C)C)c2nc(OC)ccc2[N+](=O)[O-])ccc(C(F)(F)F)c1F]

Following the procedure outlined in Example 27, Step A, substituting Int-2 with Int-2f, stirring the reaction mixture at 80° C. for 16 h, methyl 6-((2-(3-((tert-butoxycarbonyl)(6-methoxy-3-nitropyridin-2-yl)amino)prop-1-yn-1-yl)-4-fluorophenyl)amino)-2-fluoro-3-(trifluoromethyl)benzoate 33a (4.43 g, 73% yield) was prepared as a white solid. HPLC/MS 1.64 min (B), [M+H]⁺ 537.0. ¹H NMR (CDCl₃, 400 MHz) δ 9.40 (br s, 1H), 8.16 (d, J=8.8 Hz, 1H), 7.40 (t, J=8.1 Hz, 1H), 7.24 (dd, J=9.1, 5.1 Hz, 1H), 7.18 (dd, J=8.3, 2.9 Hz, 1H), 6.97-7.11 (m, 1H), 6.74 (d, J=8.8 Hz, 1H), 6.59 (d, J=8.8 Hz, 1H), 4.97 (s, 2H), 3.93 (s, 3H), 3.92 (s, 3H), 1.22-1.51 (m, 9H).

### Step B: Methyl 6-((2-(3-((3-amino-6-methoxypyridin-2-yl)(tert-butoxycarbonyl)-amino)propyl)-4-fluorophenyl)amino)-2-fluoro-3-(trifluoromethyl)benzoate 33b

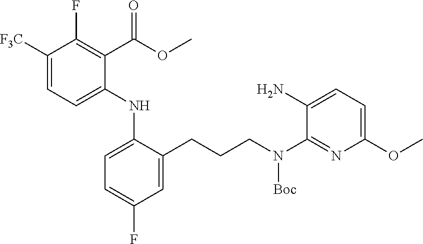 [SMILES: COC(=O)c1c(Nc2ccc(F)cc2CCCN(C(=O)OC(C)(C)C)c2nc(OC)ccc2N)ccc(C(F)(F)F)c1F]

Following the procedure outlined in Example 27, Step C, substituting EtOH with MeOH/EtOAc and stirring the reaction mixture at room temperature for 6 h, methyl 6-((2-(3-((3-amino-6-methoxypyridin-2-yl)(tert-butoxycarbonyl)amino)propyl)-4-fluorophenyl)amino)-2-fluoro-3-(trifluoromethyl)benzoate 33b (4.08 g, 96% yield) was prepared as a light pink solid. HPLC/MS 1.57 min (B), [M+H]⁺ 611.1. ¹H NMR (CDCl₃, 400 MHz) δ 9.27 (s, 1H), 7.28-7.35 (m, 1H), 7.14 (dd, J=8.8, 5.4 Hz, 1H), 6.92-7.05 (m, 3H), 6.51 (d, J=8.3 Hz, 1H), 6.31 (d, J=8.8 Hz, 1H), 3.98 (s, 3H), 3.75-3.81 (m, 5H), 2.56-2.61 (m, 2H), 1.79-1.87 (m, 2H), 1.45 (s, 9H).

### Step C: 6-((2-(3-((3-Amino-6-methoxypyridin-2-yl)(tert-butoxycarbonyl)amino)propyl)-4-fluorophenyl) amino)-2-fluoro-3-(trifluoromethyl)benzoic acid 33c

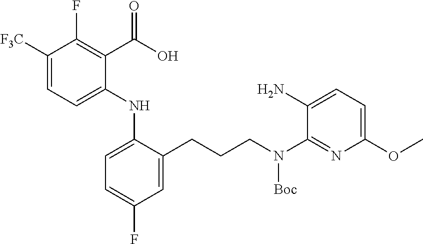 [SMILES: COc1ccc(N)c(N(CCCc2cc(F)ccc2Nc2ccc(C(F)(F)F)c(F)c2C(=O)O)C(=O)OC(C)(C)C)n1]

Following the procedure outlined in Example 27, Step B, stirring the reaction mixture in THF/MeOH/H₂O at room temperature for 2 h, 6-((2-(3-((3-amino-6-methoxypyridin-2-yl)(tert-butoxycarbonyl) amino)propyl)-4-fluorophenyl)amino)-2-fluoro-3-(trifluoromethyl)benzoic acid 33c (3.64 g, 89% yield) was prepared as an orange solid. HPLC/MS 1.01 min (B), [M+H]⁺ 597.1. ¹H NMR (CDCl₃, 400 MHz) δ 7.29-7.37 (m, 1H), 7.09-7.21 (m, 1H), 6.89-7.03 (m, 2H), 6.83 (br s, 1H), 6.56 (d, J=8.8 Hz, 1H), 6.43 (d, J=8.8 Hz, 1H), 3.87-4.04 (m, 2H), 3.80 (s, 3H), 2.48-2.68 (m, 2H), 1.74-1.94 (m, 2H), 1.45 (s, 8H).

### Step D: tert-Butyl 1,8-difluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate 33d

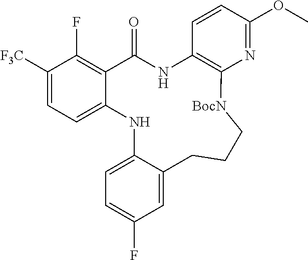 [SMILES: COc1ccc2c(n1)N(C(=O)OC(C)(C)C)CCCc1cc(F)ccc1Nc1ccc(C(F)(F)F)c(F)c1C(=O)N2]

Following the procedure outlined in Example 27, Step D, stirring the reaction mixture in MeCN at room temperature for 3 h, tert-butyl 1,8-difluoro-15-methoxy-19-oxo-2-(trifluoromethyl)-5,10,11,12,18,19-hexahydro-13H-dibenzo[f,i]pyrido[2,3,-b][1,4,8]triazacyclotridecine-13-carboxylate 33d (2.79 g, 78% yield) was prepared as a white solid. HPLC/MS 1.59 min (B), [M+H]⁺ 579.0. ¹H NMR (CDCl₃, 400 MHz) δ 8.30 (br s, 1H) 7.84 (br d, J=8.3 Hz, 1H), 7.66 (br s, 1H), 7.24-7.36 (m, 2H), 6.95-7.08 (m, 2H), 6.80 (d, J=8.3 Hz, 1H), 6.30 (br d, J=8.8 Hz, 1H), 4.23-4.42 (m, 1H), 3.89-3.98 (m, 4H), 2.53-2.70 (m, 1H), 2.20-2.35 (m, 1H), 1.47-1.56 (m, 2H).

### Step E: tert-Butyl 1,8-difluoro-15-methoxy-19-oxo-2-(trifluoromethyl)-11,12-dihydro-19H-5,18-methano dibenzo[f,i]pyrido[2,3,-b][1,4,8]triazacyclotridecine-13(10H)-carboxylate 33e

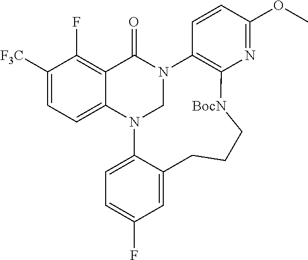 [SMILES: COc1ccc2c(n1)N(C(=O)OC(C)(C)C)CCCc1cc(F)ccc1N1CN2C(=O)c2c1ccc(C(F)(F)F)c2F]

Following the procedure outlined in Example 27, Step E, stirring the reaction mixture at 90° C. for 22.5 h, tert-butyl 1,8-difluoro-15-methoxy-19-oxo-2-(trifluoromethyl)-11,12-dihydro-19H-5,18-methanodibenzo [f,i]pyrido[2,3,-b][1,4,8]triazacyclotridecine-13(10H)-carboxylate 33e (2.12 g 73% yield) was prepared as a white solid. HPLC/MS 1.45 min (B), [M+H]⁺ 591.1. ¹H NMR (CDCl₃, 400 MHz) δ 7.78 (br s, 1H), 7.44 (br t, J=7.8 Hz, H), 7.14-7.23 (m, 2H), 7.00 (td, J=8.1, 2.9 Hz, 1H), 6.75 (d, J=8.31 Hz, 1H), 6.29 (d, J=8.8 Hz, 1H), 5.26 (br d, J=11.3 Hz, 1H), 4.35-4.66 (m, 1H), 3.77-3.99 (m, 4H), 3.14-3.57 (m, 1H), 2.48-2.75 (m, 2H), 1.94 (br s, 1H), 0.98-1.37 (m, 10H).

### Step F: 1,8-Difluoro-2-(trifluoromethyl)-10,11,12,13-tetrahydro-19H-5,18-methanodibenzo [f,i]pyrido[2,3,-b][1,4,8]triazacyclotridecine-15,19(14H)-dione

### Example 33

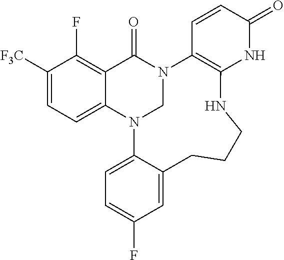 [SMILES: O=C1c2c(ccc(C(F)(F)F)c2F)N2CN1c1ccc(=O)[nH]c1NCCCc1cc(F)ccc12]

Following the procedure outlined in Example 27, Step F, stirring the reaction mixture at 90° C. for 15.5 h, to which was additional 5N HCl in IPA (42 equiv) was added and heating continued at 90° C. for another 7 h, 1,8-difluoro-2-(trifluoromethyl)-10,11,12,13-tetrahydro-19H-5,18-methanodibenzo[f,i]pyrido[2,3,-b][1,4,8]triazacyclotridecine-15,19(14H)-dione (593 mg, 35% yield) was prepared as a white solid. HPLC/MS 1.01 min (B), [M+H]⁺ 477.0. ¹H NMR (DMSO-d₆, 400 MHz) δ 7.61 (t, J=7.3 Hz, 1H), 7.27-7.42 (m, 3H), 7.21 (td, J=8.4, 3.2 Hz, 1H), 6.14 (d, J=8.8 Hz, 1H), 5.77 (br d, J=8.8 Hz, 1H), 5.60 (dd, J=8.1, 5.4 Hz, 1H), 5.47 (d, J=11.3 Hz, 1H), 4.48 (d, J=11.3 Hz, 1H), 3.34 (br d, J=5.9 Hz, 1H), 2.93-3.08 (m, 1H).

### Example 34

### 8-Fluoro-2-(trifluoromethyl)-10,11,12,13-tetrahydro-19H-5,18-methanodibenzo[i]pyrido[2,3,-b:3′,2′-f][1,4,8]triazacyclotridecine-15,19(14H)-dione

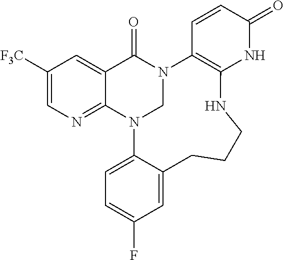 [SMILES: O=C1c2cc(C(F)(F)F)cnc2N2CN1c1ccc(=O)[nH]c1NCCCc1cc(F)ccc12]

### Step A: Methyl 2-((2-(3-((tert-butoxycarbonyl)amino)prop-1-yn-1-yl)-4-fluorophenyl)-amino)-5-(trifluoromethyl)nicotinate 34a

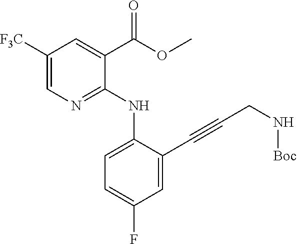 [SMILES: COC(=O)c1cc(C(F)(F)F)cnc1Nc1ccc(F)cc1C#CCNC(=O)OC(C)(C)C]

Following the procedure outlined in Example 27, Step A, substituting methyl 2-bromo-4-(trifluoromethyl)benzoate with methyl 2-chloro-5-(trifluoromethyl)nicotinate, Pd(OAc)₂ with Pd₂(dba)₃, and stirring at 85° C. for 20 h, methyl 2-((2-(3-((tert-butoxycarbonyl)amino)prop-1-yn-1-yl)-4-fluorophenyl)amino)-5-(trifluoromethyl)nicotinate 34a (830 mg, 13% yield) was prepared as a light yellow solid. HPLC/MS 1.53 min (A), [M+H]⁺ 468.1. ¹H NMR (DMSO-d₆, 400 MHz) δ 10.8-11.0 (m, 1H), 8.8-9.1 (m, 1H), 8.5-8.6 (m, 1H), 8.4-8.5 (m, 1H), 7.33 (br dd, 3H, J=6.4, 8.8 Hz), 6.2-6.5 (m, 1H), 5.1-5.4 (m, 1H), 4.1-4.2 (m, 1H), 3.8-4.0 (m, 3H), 1.3-1.4 (m, 9H).

### Step B: 2-((2-(3-((tert-Butoxycarbonyl)amino)prop-1-yn-1-yl)-4-fluorophenyl)amino)-5-(trifluoromethyl) nicotinic acid 34b

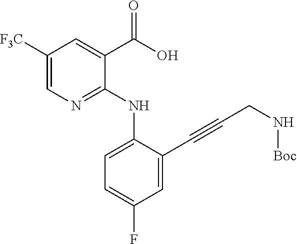 [SMILES: CC(C)(C)OC(=O)NCC#Cc1cc(F)ccc1Nc1ncc(C(F)(F)F)cc1C(=O)O]

Following the procedure outlined in Example 27, Step B, stirring the reaction mixture in THF/MeOH/H₂O and heating at 75° C. for 1 h, 2-((2-(3-((tert-butoxycarbonyl)amino)prop-1-yn-1-yl)-4-fluorophenyl)amino)-5-(trifluoromethyl)nicotinic acid 34b (803 mg, 75% yield) was prepared as an off-solid. HPLC/MS 1.45 min (A), [M+H]⁺ 454.1. ¹H NMR (DMSO-d₆, 400 MHz) 11.17 (s, 1H), 8.7-8.8 (m, 1H), 8.6-8.7 (m, 1H), 8.4-8.5 (m, 1H), 7.2-7.4 (m, 3H), 4.1-4.2 (m, 2H), 3.1-3.5 (m, 1H), 1.39 (s, 8H), 1.3-1.5 (m, 1H).

### Step C: tert-Butyl (3-(2-((3-((2-bromo-6-methoxypyridin-3-yl)carbamoyl)-5-(trifluoro-methyl)pyridin-2-yl)amino)-5-fluorophenyl)prop-2-yn-1-yl)carbamate 34c

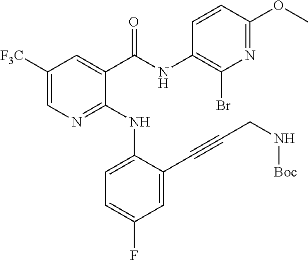 [SMILES: COc1ccc(NC(=O)c2cc(C(F)(F)F)cnc2Nc2ccc(F)cc2C#CCNC(=O)OC(C)(C)C)c(Br)n1]

Following the procedure outlined in Example 28, Step D, replacing DMF with MeCN and stirring the reaction mixture at room temperature for 3 h, tert-butyl (3-(2-((3-((2-bromo-6-methoxypyridin-3-yl)carbamoyl)-5-(trifluoromethyl)pyridin-2-yl)amino)-5-fluorophenyl)prop-2-yn-1-yl)carbamate 34c (425 mg, 36% yield) was prepared as a yellow solid. HPLC/MS 1.55 min (A), [M+H]⁺ 637.9. ¹H NMR (CDCl₃, 400 MHz) δ 11.33 (s, 1H), 10.71 (s, 1H), 8.8-8.8 (m, 1H), 8.7-8.7 (m, 1H), 8.5-8.6 (m, 1H), 7.8-8.0 (m, 1H), 7.2-7.4 (m, 2H), 6.96 (d, 2H, J=8.3 Hz), 4.0-4.1 (m, 2H), 3.90 (s, 3H), 1.36 (s, 9H).

### Step D tert-Butyl (3-(2-((3-((2-bromo-6-methoxypyridin-3-yl)carbamoyl)-5-(trifluoro-methyl)pyridin-2-yl)amino)-5-fluorophenyl)propyl)carbamate 34d

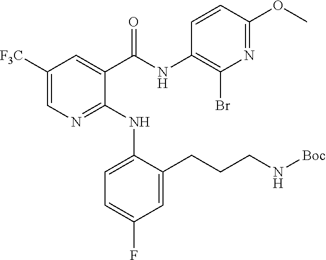 [SMILES: COc1ccc(NC(=O)c2cc(C(F)(F)F)cnc2Nc2ccc(F)cc2CCCNC(=O)OC(C)(C)C)c(Br)n1]

Following the procedure outlined in Example 28, Step C, replacing EtOH with EtOAc and stirring the reaction mixture at room temperature for 3 days, tert-butyl (3-(2-((3-((2-bromo-6-methoxypyridin-3-yl)carbamoyl)-5-(trifluoromethyl)pyridin-2-yl)amino)-5-fluorophenyl)propyl)carbamate 34d (2.79 g, 78% yield) was prepared as a light yellow oil. HPLC/MS 1.43 min (A), [M+H]⁺ 642.0.

### Step E: tert-Butyl (3-(2-(3-(2-bromo-6-methoxypyridin-3-yl)-4-oxo-6-(trifluoromethyl)-3,4-dihydropyrido [2,3-d]pyrimidin-1(2H)-yl)-5-fluorophenyl)propyl)carbamate 34e

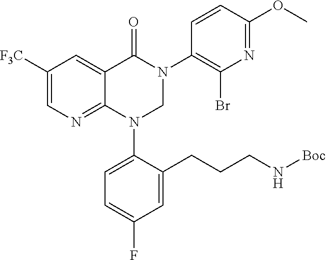 [SMILES: COc1ccc(N2CN(c3ccc(F)cc3CCCNC(=O)OC(C)(C)C)c3ncc(C(F)(F)F)cc3C2=O)c(Br)n1]

Following the procedure outlined in Example 28, Step E, stirring the reaction mixture at 80° C. in MeCN for 20 h, tert-butyl (3-(2-(3-(2-bromo-6-methoxypyridin-3-yl)-4-oxo-6-(trifluoromethyl)-3,4-dihydropyrido [2,3-d]pyrimidin-1(2H)-yl)-5-fluorophenyl)propyl)carbamate 34e (130 mg, 28% yield) was prepared as a white solid. HPLC/MS 1.32 min (A), [M+H]⁺ 653.9.

### Step F: 1-(2-(3-aminopropyl)-4-fluorophenyl)-3-(2-bromo-6-methoxypyridin-3-yl)-6-(trifluoromethyl)-2,3-dihydropyrido[2,3-d]pyrimidin-4(1H)-one, hydrochloride salt 34f

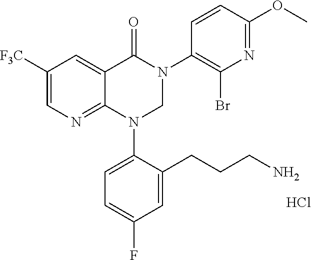 [SMILES: COc1ccc(N2CN(c3ccc(F)cc3CCCN)c3ncc(C(F)(F)F)cc3C2=O)c(Br)n1 ; Cl]

Following the procedure outlined in Example 28, Step F, dissolving the intermediate in dioxane and adding conc. HCl and stirring the reaction mixture at 50° C. for 5 h, 1-(2-(3-aminopropyl)-4-fluorophenyl)-3-(2-bromo-6-methoxypyridin-3-yl)-6-(trifluoromethyl)-2,3-dihydropyrido[2,3-d]pyrimidin-4(1H)-one, hydrochloride salt 34f (145 mg, 97% yield) was prepared as a light yellow solid. HPLC/MS 0.82 min (A), [M+H]⁺ 554.0.

### Step G: 8-Fluoro-15-methoxy-2-(trifluoromethyl)-10,11,12,13-tetrahydro-19H-5,18-methanodibenzo[i]dipyrido[2,3,-b:3′,2′-f][1,4,8]triazacyclotridecine-19-one 342

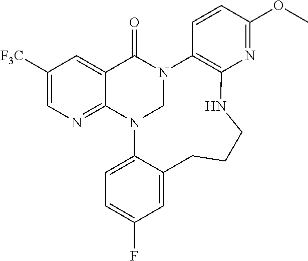 [SMILES: COc1ccc2c(n1)NCCCc1cc(F)ccc1N1CN2C(=O)c2cc(C(F)(F)F)cnc21]

Following the procedure outlined in Example 28, Step G, stirring the reaction mixture at 100° C. for 3 h, 8-fluoro-15-methoxy-2-(trifluoromethyl)-10,11,12,13-tetrahydro-19H-5,18-methanodibenzo[i]dipyrido [2,3,-b:3′,2′-f][1,4,8]triazacyclotridecine-19-one 34g (24 mg, 19% yield) was prepared as a light yellow foam. HPLC/MS 1.13 min (A), [M+H]⁺ 474.1. ¹H NMR (CD₃OD, 400 MHz) δ 8.4-8.6 (m, 2H), 7.50 (d, 1H, J=8.3 Hz), 7.3-7.4 (m, 1H), 7.2-7.3 (m, 1H), 7.07 (d, 1H, J=2.9 Hz), 6.18 (d, 1H, J=8.3 Hz), 5.5-5.7 (m, 1H), 4.4-4.6 (m, 1H), 3.86 (s, 3H), 3.7-3.8 (m, 1H), 3.1-3.3 (m, 1H), 2.9-3.1 (m, 1H), 2.6-2.7 (m, 1H), 2.1-2.2 (m, 1H), 1.4-1.6 (m, 1H).

### Step H: 8-Fluoro-2-(trifluoromethyl)-10,11,12,13-tetrahydro-19H-5,18-methanodibenzo[i]dipyrido[2,3,-b:3′,2′-f][1,4,8]triazacyclotridecine-15,19(14H)-dione

### Example 34

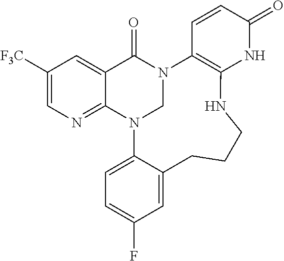 [SMILES: O=C1c2cc(C(F)(F)F)cnc2N2CN1c1ccc(=O)[nH]c1NCCCc1cc(F)ccc12]

Following the procedure outlined in Example 28, and using Step H, stirring the reaction mixture at 100° C. for 24 h, 8-fluoro-2-(trifluoromethyl)-10,11,12,13-tetrahydro-19H-5,18-methanodibenzo [i]dipyrido[2,3,-b:3′,2′-f][1,4,8]triazacyclotridecine-15,19(14H)-dione (12.3 mg, 52% yield) was prepared as a yellow solid. HPLC/MS 0.85 min (A), [M+H]⁺ 460.1. ¹H NMR (CD₃OD, 400 MHz) δ 8.51 (d, J=2.4 Hz, 1H), 8.46 (dd, J=1.0, 2.4 Hz, 1H), 7.45 (d, J=8.8 Hz, 1H), 7.36 (dd, J=5.4, 8.8 Hz, 1H), 7.19 (dd, J=2.9, 9.3 Hz, 1H), 7.07 (dt, J=2.9, 8.6 Hz, 1H), 5.94 (d, J=9.3 Hz, 1H), 5.60 (d, J=11.7 Hz, 1H), 4.68 (d, J=11.7 Hz, 1H), 3.52-3.45 (m, 1H), 3.13 (ddd, J=4.6, 9.0, 14.2 Hz, 1H), 2.88-2.83 (m, 1H), 2.60-2.52 (m, 1H), 2.15-2.05 (m, 1H), 1.54-1.46 (m, 1H).

### Example 35

### 8-Fluoro-2-(trifluoromethyl)-10,11,12,13-tetrahydro-19H-5,18-methanodibenzo[i]dipyrido[2,3,-b:4′,3′-f][1,4,8]triazacyclotridecine-15,19(14H)-dione

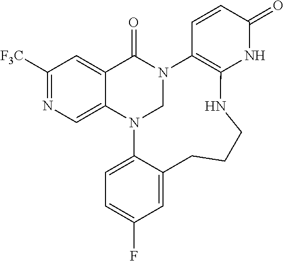 [SMILES: O=C1c2cc(C(F)(F)F)ncc2N2CN1c1ccc(=O)[nH]c1NCCCc1cc(F)ccc12]

### Step A: Methyl 5-((2-(3-((tert-butoxycarbonyl)amino)propyl)-4-fluorophenyl)amino)-2-(trifluoromethyl) isonicotinate 35a

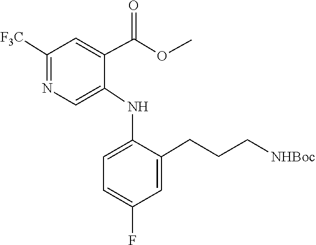 [SMILES: COC(=O)c1cc(C(F)(F)F)ncc1Nc1ccc(F)cc1CCCNC(=O)OC(C)(C)C]

Following the procedure outlined in Example 28, Step A, substituting methyl 2-bromo-4-(trifluoromethyl)benzoate with methyl 5-bromo-2-(trifluoromethyl)isonicotinate, Int-3e with Int-3g, Pd(OAc)₂ with Pd₂(dba)₃, dioxane with toluene, and stirring the reaction mixture at 85° C. for h, methyl 5-((2-(3-((tert-butoxycarbonyl)amino)propyl)-4-fluorophenyl)amino)-2-(trifluoromethyl)isonicotinate 35a (3.0 g, 77% yield) was prepared as a yellow solid. HPLC/MS 1.32 min (A), [M+H]⁺ 472.1. ¹H NMR (CDCl₃, 400 MHz) δ 9.25 (s, 1H), 8.20 (s, 1H), 8.11 (s, 1H), 7.29 (s, 1H), 7.1-7.1 (m, 1H), 7.0-7.0 (m, 1H), 4.4-4.7 (m, 1H), 4.03 (s, 3H), 3.1-3.3 (m, 2H), 2.5-2.7 (m, 2H), 1.7-1.9 (m, 2H), 1.42 (s, 9H).

### Step B: 5-((2-(3-((tert-Butoxycarbonyl)amino)propyl)-4-fluorophenyl)amino)-2-(trifluoromethyl) isonicotinic acid 35b

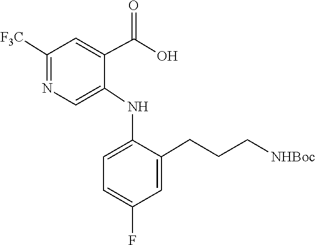 [SMILES: CC(C)(C)OC(=O)NCCCc1cc(F)ccc1Nc1cnc(C(F)(F)F)cc1C(=O)O]

Following the procedure outlined in Example 28, Step B, stirring the reaction mixture in THF/MeOH/H₂O and heating at 50° C. for 1 h, 5-((2-(3-((tert-butoxycarbonyl)amino)propyl)-4-fluorophenyl)amino)-2-(trifluoromethyl)isonicotinic acid 35b (2.97 g, 97% yield) was prepared as a yellow solid. HPLC/MS 1.22 min (A), [M+H]⁺ 458.1. ¹H NMR (DMSO-d₆, 400 MHz) δ 13.7-14.6 (m, 1H), 9.46 (s, 1H), 8.03 (d, 2H, J=14.7 Hz), 7.43 (dd, 1H, J=5.6, 8.6 Hz), 7.26 (dd, 1H, J=2.9, 9.8 Hz), 7.15 (d, 1H, J=2.9 Hz), 6.7-6.8 (m, 1H), 2.8-3.0 (m, 2H), 2.55 (t, 2H, J=7.6 Hz), 1.6-1.7 (m, 2H), 1.32 (s, 9H).

### Step C: tert-Butyl (3-(2-((4-((2-bromo-6-methoxypyridin-3-yl)carbamoyl)-6-(trifluoro-methyl)pyridin-3-yl)amino)-5-fluorophenyl)propyl)carbamate 35c

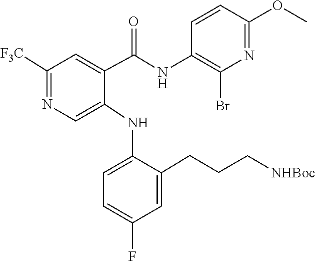 [SMILES: COc1ccc(NC(=O)c2cc(C(F)(F)F)ncc2Nc2ccc(F)cc2CCCNC(=O)OC(C)(C)C)c(Br)n1]

Following the procedure outlined in Example 28, Step D, replacing DMF with MeCN and stirring the reaction mixture at room temperature for 3 days, tert-butyl (3-(2-((4-((2-bromo-6-methoxypyridin-3-yl)carbamoyl)-6-(trifluoromethyl)pyridin-3-yl)amino)-5-fluorophenyl)propyl)carbamate 35c (1.12 g, 52% yield) was prepared as an orange solid. HPLC/MS 1.40 min (A), [M+H]⁺ 642.1. ¹H NMR (DMSO-d₆, 400 MHz) δ 10.67 (s, 1H), 9.4-9.7 (m, 1H), 8.28 (s, 1H), 8.13 (s, 1H), 7.87 (d, 1H, J=8.8 Hz), 7.4-7.5 (m, 1H), 7.2-7.3 (m, 1H), 7.1-7.2 (m, 1H), 6.97 (d, 1H, J=8.8 Hz), 6.7-6.8 (m, 1H), 3.89 (s, 3H), 2.8-2.9 (m, 2H), 2.5-2.6 (m, 2H), 1.62 (s, 2H), 1.32 (s, 9H).

### Step D: tert-Butyl (3-(2-(3-(2-bromo-6-methoxypyridin-3-yl)-4-oxo-6-(trifluoromethyl)-3,4-dihydro pyrido[3,4-d]pyrimidin-1(2H)-yl)-5-fluorophenyl)propyl)carbamate 35d

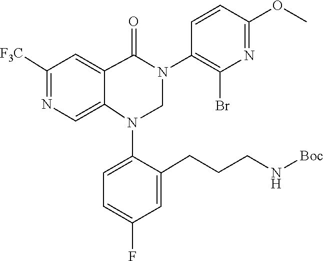 [SMILES: COc1ccc(N2CN(c3ccc(F)cc3CCCNC(=O)OC(C)(C)C)c3cnc(C(F)(F)F)cc3C2=O)c(Br)n1]

Following the procedure outlined in Example 28, Step E, stirring the reaction mixture at 80° C. in MeCN for 40 h, tert-butyl (3-(2-(3-(2-bromo-6-methoxypyridin-3-yl)-4-oxo-6-(trifluoromethyl)-3,4-dihydropyrido[3,4-d]pyrimidin-1(2H)-yl)-5-fluorophenyl)propyl)carbamate 35d (730 mg, 61% yield) was prepared as a light yellow foam. HPLC/MS 1.33 min (A), [M+H]+ 654.0. ¹H NMR (CDCl₃, 400 MHz) δ 8.28 (s, 1H), 7.82 (br s, 1H), 7.54 (br s, 1H), 7.3-7.4 (m, 1H), 7.0-7.3 (m, 3H), 6.82 (d, 1H, J=8.3 Hz), 5.2-5.5 (m, 1H), 4.95 (d, 1H, J=9.8 Hz), 4.53 (br s, 1H), 3.98 (s, 3H), 3.1-3.4 (m, 2H), 2.5-2.9 (m, 2H), 1.7-2.0 (m, 2H), 1.41 (br s, 9H).

### Step E: 1-(2-(3-Aminopropyl)-4-fluorophenyl)-3-(2-bromo-6-methoxypyridin-3-yl)-6-(trifluoromethyl)-2,3-dihydropyrido[3,4-d]pyrimidin-4(1H)-one, hydrochloride salt 35e

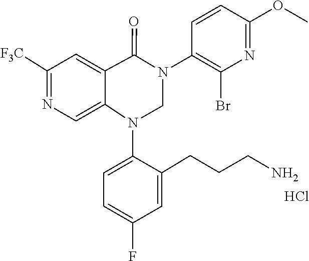 [SMILES: COc1ccc(N2CN(c3ccc(F)cc3CCCN)c3cnc(C(F)(F)F)cc3C2=O)c(Br)n1 ; Cl]

Following the procedure outlined in Example 28, Step F, dissolving the intermediate in dioxane and adding conc. HCl and stirring the reaction mixture at 50° C. for 3 h, 1-(2-(3-aminopropyl)-4-fluorophenyl)-3-(2-bromo-6-methoxypyridin-3-yl)-6-(trifluoromethyl)-2,3-dihydropyrido[3,4-d]pyrimidin-4(1H)-one, hydrochloride salt 35e (681 mg, 98% yield) was prepared as a light yellow foam. HPLC/MS 0.84 min (A), [M+H]⁺ 554.0.

### Step F: 8-Fluoro-15-methoxy-2-(trifluoromethyl)-10,11,12,13-tetrahydro-19H-5,18-methanodibenzo[i]dipyrido[2,3,-b:4′,3′-f][1,4,8]triazacyclotridecine-19-one 35f

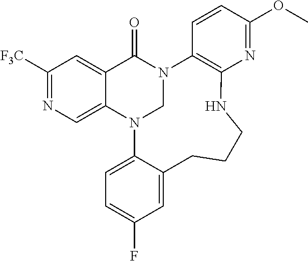 [SMILES: COc1ccc2c(n1)NCCCc1cc(F)ccc1N1CN2C(=O)c2cc(C(F)(F)F)ncc21]

Following the procedure outlined in Example 28, Step G, stirring the reaction mixture at 100° C. for 3 h, 8-fluoro-15-methoxy-2-(trifluoromethyl)-10,11,12,13-tetrahydro-19H-5,18-methanodibenzo[i]dipyrido [2,3,-b:4′,3′-f][1,4,8]triazacyclotridecine-19-one 35f (96 mg, 20% yield) was prepared as a yellow solid. HPLC/MS 1.14 min (A), [M+H]⁺ 474.2. ¹H NMR (CD₃OD, 400 MHz) δ 8.21 (s, 1H), 7.88 (s, 1H), 7.50 (d, 1H, J=8.3 Hz), 7.4-7.5 (m, 1H), 7.31 (dd, 1H, J=2.9, 9.3 Hz), 7.16 (dt, 1H, J=3.2, 8.4 Hz), 6.18 (d, 1H, J=8.3 Hz), 5.55 (d, 1H, J=11.2 Hz), 4.52 (d, 1H, J=11.2 Hz), 3.86 (s, 3H), 3.7-3.8 (m, 1H), 3.1-3.2 (m, 1H), 2.9-3.1 (m, 1H), 2.71 (d, 1H, J=8.4, 13.9 Hz), 2.1-2.3 (m, 1H), 1.48 (br dd, 1H, J=4.9, 9.3 Hz).

### Step G: 8-Fluoro-2-(trifluoromethyl)-10,11,12,13-tetrahydro-19H-5,18-methanodibenzo[i]dipyrido[2,3,-b:4′,3′-f][1,4,8]triazacyclotridecine-15,19(14H)-dione

### Example 35

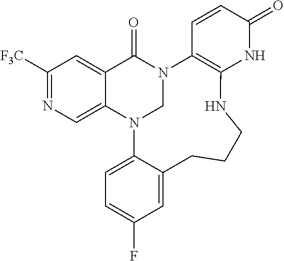 [SMILES: O=C1c2cc(C(F)(F)F)ncc2N2CN1c1ccc(=O)[nH]c1NCCCc1cc(F)ccc12]

Following the procedure outlined in Example 28, Step H, stirring the reaction mixture at 80° C. for 24 h, 8-fluoro-2-(trifluoromethyl)-10,11,12,13-tetrahydro-19H-5,18-methanodibenzo[i]dipyrido[2,3,-b:4′,3′-f][1,4,8]triazacyclotridecine-15,19(14H)-dione (58 mg, 63% yield) was prepared as a yellow solid. HPLC/MS 0.87 min (A), [M+H]⁺ 460.1. ¹H NMR (CD₃OD, 400 MHz) δ 10.77 (br s, 1H), 7.98 (s, 1H), 7.73 (s, 1H), 7.48 (dd, J=5.4, 8.8 Hz, 1H), 7.30-7.24 (m, 2H), 7.15 (dt, J=2.9, 8.6 Hz, 1H), 5.80 (dd, J=4.4, 9.3 Hz, 1H), 5.72-5.70 (m, 1H), 5.43 (d, J=11.2 Hz, 1H), 4.44 (d, J=10.8 Hz, 1H), 3.42 (br dd, J=9.3, 14.2 Hz, 1H), 2.89-2.82 (m, 2H), 1.91 (br dd, J=4.6, 9.0 Hz, 1H), 1.26 (br d, J=6.4 Hz, 1H).

### Example 36

### 8-Fluoro-3-(trifluoromethyl)-10,11,12,13-tetrahydro-19H-5,18-methanodibenzo[i]dipyrido[2,3,-b:3′,4′-f][1,4,8]triazacyclotridecine-15,19(14H)-dione

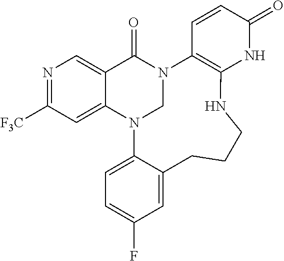 [SMILES: O=C1c2cnc(C(F)(F)F)cc2N2CN1c1ccc(=O)[nH]c1NCCCc1cc(F)ccc12]

### Step A: Ethyl 4-((2-(3-((tert-butoxycarbonyl)amino)propyl)-4-fluorophenyl)amino)-6-(trifluoromethyl) nicotinate 36a

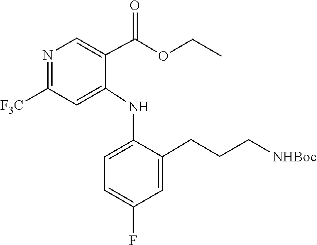 [SMILES: CCOC(=O)c1cnc(C(F)(F)F)cc1Nc1ccc(F)cc1CCCNC(=O)OC(C)(C)C]

Following the procedure outlined in Example 28, Step A, substituting methyl 2-bromo-4-(trifluoromethyl)benzoate with ethyl 4-chloro-6-(trifluoromethyl)nicotinate, Int-3e with Int-3g, Pd(OAc)₂ with Pd₂(dba)₃, and stirring the reaction mixture at 85° C. for 20 h, ethyl 4-((2-(3-((tert-butoxycarbonyl)amino)propyl)-4-fluorophenyl)amino)-6-(trifluoromethyl)nicotinate 36a (2.41 g, 83% yield) was prepared as a yellow foam. HPLC/MS 1.31 min (A), [M+H]⁺ 486.2. ¹H NMR (CD₃OD, 400 MHz) δ 8.98 (s, 1H), 7.3-7.4 (m, 1H), 7.2-7.3 (m, 1H), 7.1-7.2 (m, 1H), 6.76 (s, 1H), 4.4-4.6 (m, 2H), 3.0-3.1 (m, 2H), 2.5-2.7 (m, 2H), 1.6-1.8 (m, 2H), 1.47 (t, 3H, J=7.1 Hz), 1.39 (s, 9H).

### Step B: 4-((2-(3-((tert-Butoxycarbonyl)amino)propyl)-4-fluorophenyl)amino)-6-(trifluoromethyl) nicotinate 36b

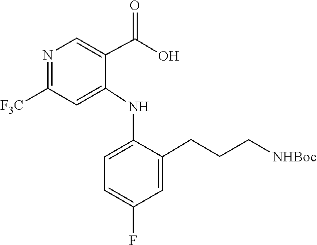 [SMILES: CC(C)(C)OC(=O)NCCCc1cc(F)ccc1Nc1cc(C(F)(F)F)ncc1C(=O)O]

Following the procedure outlined in Example 28, Step B, stirring the the reaction mixture in THF/MeOH/H₂O and heating at 50° C. for 2 h, 4-((2-(3-((tert-butoxycarbonyl)amino)propyl)-4-fluorophenyl)amino)-6-(trifluoromethyl)nicotinate 36b (2.29 g, 96% yield) was prepared as a yellow foam. HPLC/MS 1.13 min (A), [M+H]⁺ 458.1. ¹H NMR (CDCl₃, 400 MHz) δ 10.07 (br s, 1H), 9.1-9.4 (m, 1H), 7.2-7.3 (m, 1H), 7.0-7.1 (m, 2H), 6.8-6.9 (m, 1H), 4.5-6.3 (m, 1H), 3.0-3.4 (m, 2H), 2.5-2.8 (m, 2H), 1.79 (br s, 2H), 1.45 (s, 9H).

### Step C: tert-Butyl (3-(2-((5-((2-bromo-6-methoxypyridin-3-yl)carbamoyl)-2-(trifluoro-methyl)pyridin-3-yl)amino)-5-fluorophenyl)propyl)carbamate 36c

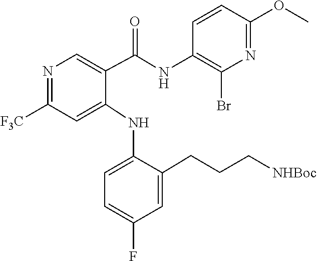 [SMILES: COc1ccc(NC(=O)c2cnc(C(F)(F)F)cc2Nc2ccc(F)cc2CCCNC(=O)OC(C)(C)C)c(Br)n1]

Following the procedure outlined in Example 28, Step D, replacing DMF with MeCN and stirring the reaction mixture at room temperature for 3 days, tert-butyl (3-(2-((5-((2-bromo-6-methoxypyridin-3-yl)carbamoyl)-2-(trifluoromethyl)pyridin-3-yl)amino)-5-fluorophenyl)propyl)carbamate 36c (1.34 g, 37% yield) was prepared as an orange foam. HPLC/MS 1.33 min (A), [M+H]⁺ 642.0. ¹H NMR (CDCl₃, 400 MHz) δ 9.89 (s, 1H), 8.89 (s, 1H), 8.48 (d, J=8.8 Hz, 1H), 8.31 (br s, 1H), 7.22-7.26 (m, 1H), 7.02-7.11 (m, 2H), 6.91 (s, 1H), 6.82 (d, J=8.8 Hz, 1H), 3.98 (s, 3H), 3.17 (br d, J=5.9 Hz, 2H), 2.60-2.64 (m, 2H), 1.77 (quin, J=7.5 Hz, 2H), 1.41 (s, 9H).

### Step D: tert-Butyl (3-(2-(3-(2-bromo-6-methoxypyridin-3-yl)-4-oxo-7-(trifluoromethyl)-3,4-dihydro pyrido[4,3-d]pyrimidin-1(2H)-yl)-5-fluorophenyl)propyl)carbamate 36d

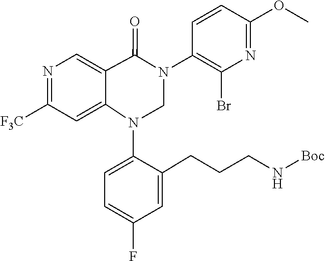 [SMILES: COc1ccc(N2CN(c3ccc(F)cc3CCCNC(=O)OC(C)(C)C)c3cc(C(F)(F)F)ncc3C2=O)c(Br)n1]

Following the procedure outlined in Example 28, Step E, stirring the reaction mixture at room temperature in MeCN for 20 h, tert-butyl (3-(2-(3-(2-bromo-6-methoxypyridin-3-yl)-4-oxo-7-(trifluoromethyl)-3,4-dihydropyrido[4,3-d]pyrimidin-1(2H)-yl)-5-fluorophenyl)propyl)carbamate 36d (399 mg, 26% yield) was prepared as a yellow foam, in 85% purity. HPLC/MS 1.30 min (A), [M+H]⁺ 653.9.

### Step E: 1-(2-(3-Aminopropyl)-4-fluorophenyl)-3-(2-bromo-6-methoxypyridin-3-yl)-7-(trifluoromethyl)-2,3-dihydropyrido[4,3-d]pyrimidin-4(1H)-one, hydrochloride salt 36e

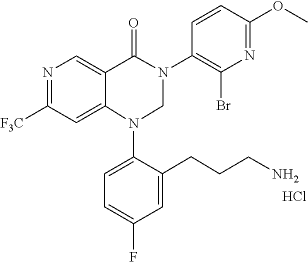 [SMILES: COc1ccc(N2CN(c3ccc(F)cc3CCCN)c3cc(C(F)(F)F)ncc3C2=O)c(Br)n1 ; Cl]

Following the procedure outlined in Example 28, Step F, dissolving the intermediate in dioxane and adding conc. HCl and stirring the reaction mixture at 50° C. for 1 h, 1-(2-(3-aminopropyl)-4-fluorophenyl)-3-(2-bromo-6-methoxypyridin-3-yl)-7-(trifluoromethyl)-2,3-dihydropyrido[4,3-d]pyrimidin-4(1H)-one, hydrochloride salt 36e (379 mg, 100% yield) was prepared as a yellow foam. HPLC/MS 0.77 min (A), [M+H]⁺ 554.0.

### Step F: 8-Fluoro-15-methoxy-3-(trifluoromethyl)-10,11,12,13-tetrahydro-19H-5,18-methanobenzo[i]dipyrido[2,3,-b:3′,4′-f][1,4,8]triazacyclotridecine-19-one 36f

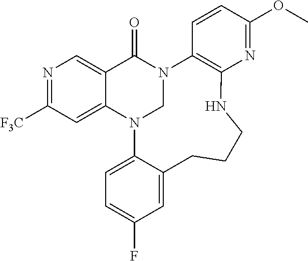 [SMILES: COc1ccc2c(n1)NCCCc1cc(F)ccc1N1CN2C(=O)c2cnc(C(F)(F)F)cc21]

Following the procedure outlined in Example 28, Step G, stirring the reaction mixture at 100° C. for 3.5 h, 8-fluoro-15-methoxy-3-(trifluoromethyl)-10,11,12,13-tetrahydro-19H-5,18-methanobenzo[i]dipyrido [2,3,-b:3′,4′-f][1,4,8]triazacyclotridecine-19-one 36f (51 mg, 22% yield) was prepared as a white solid. HPLC/MS 1.11 min (A), [M+H]⁺ 474.1. ¹H NMR (CDCl₃, 400 MHz) δ 9.24 (s, 1H), 7.47 (d, 1H, J=8.3 Hz), 7.2-7.2 (m, 2H), 7.1-7.1 (m, 1H), 6.63 (s, 1H), 6.19 (d, 1H, J=8.3 Hz), 5.45 (d, 1H, J=11.2 Hz), 5.0-5.2 (m, 1H), 4.38 (d, 1H, J=11.2 Hz), 3.88 (s, 3H), 3.6-3.8 (m, 1H), 3.2-3.4 (m, 1H), 2.7-2.9 (m, 2H), 2.1-2.4 (m, 1H), 1.5-1.6 (m, 2H).

### Step G: 8-Fluoro-3-(trifluoromethyl)-10,11,12,13-tetrahydro-19H-5,18-methanodibenzo[i]dipyrido[2,3,-b:3′,4′-f][1,4,8]triazacyclotridecine-15,19(14H)-dione

### Example 36

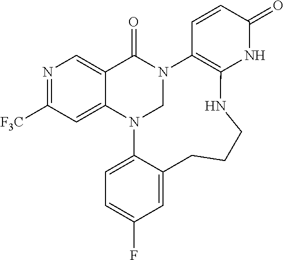 [SMILES: O=C1c2cnc(C(F)(F)F)cc2N2CN1c1ccc(=O)[nH]c1NCCCc1cc(F)ccc12]

Following the procedure outlined in Example 28, Step H, stirring the reaction mixture at 80° C. for 24 h, 8-fluoro-3-(trifluoromethyl)-10,11,12,13-tetrahydro-19H-5,18-methanodibenzo[i]dipyrido[2,3,-b:3′,4′-f][1,4,8]triazacyclotridecine-15,19(14H)-dione (26 mg, 51% yield) was prepared as a white solid. HPLC/MS 0.83 min (A), [M+H]⁺ 460.1. ¹H NMR (CD₃OD, 400 MHz) δ 10.86 (br s, 1H), 8.93 (s, 1H), 7.52 (dd, J=5.4, 8.8 Hz, 1H), 7.35-7.31 (m, 2H), 7.23 (dt, J=3.2, 8.4 Hz, 1H), 6.49 (s, 1H), 5.88 (dd, J=3.4, 9.3 Hz, 1H), 5.78 (d, J=8.8 Hz, 1H), 5.51 (d, J=11.2 Hz, 1H), 4.52 (d, J=11.2 Hz, 1H), 3.55-3.49 (m, 1H), 2.95-2.88 (m, 2H), 2.02-1.96 (m, 1H), 1.32 (br d, J=7.3 Hz, 1H).

### Example 37

### 8,9-Difluoro-2-(trifluoromethyl)-10,11,12,13-tetrahydro-19H-5,18-methanodibenzo[f,i]dipyrido[2,3,-b][1,4,8]triazacyclotridecine-15,19(14H)-dione

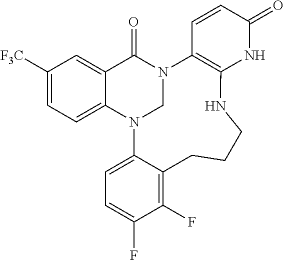 [SMILES: O=C1c2cc(C(F)(F)F)ccc2N2CN1c1ccc(=O)[nH]c1NCCCc1c2ccc(F)c1F]

### Step A: Methyl 2-((2-(3-((tert-butoxycarbonyl)amino)prop-1-yn-1-yl)-3,4-difluoro-phenyl)amino)-5-(trifluoromethyl)benzoate 37a

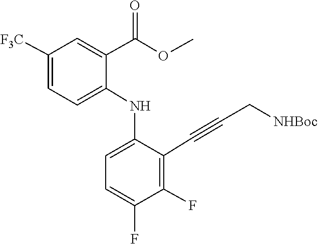 [SMILES: COC(=O)c1cc(C(F)(F)F)ccc1Nc1ccc(F)c(F)c1C#CCNC(=O)OC(C)(C)C]

Following the procedure outlined in Example 28, Step A, substituting methyl 2-bromo-4-(trifluoromethyl)benzoate with methyl 2-bromo-5-(trifluoromethyl)benzoate, Int-3e with Int-3h, Pd(OAc)₂ with Pd₂(dba)₃, dioxane with toluene, and stirring the reaction mixture at 90° C. for 20 h, methyl 2-((2-(3-((tert-butoxycarbonyl)amino)prop-1-yn-1-yl)-3,4-difluorophenyl)amino)-5-(trifluoromethyl) benzoate 37a (847 mg, 37% yield) was prepared as a yellow solid. HPLC/MS 1.51 min (A), [M-^{t}Bu]+429.1. ¹H NMR (DMSO-d₆, 400 MHz) δ 9.96 (s, 1H), 8.1-8.3 (m, 1H), 7.7-7.8 (m, 1H), 7.5-7.6 (m, 1H), 7.3-7.4 (m, 2H), 7.2-7.3 (m, 1H), 4.0-4.1 (m, 2H), 3.94 (s, 3H), 1.35 (s, 9H).

### Step B: 2-((2-(3-((tert-Butoxycarbonyl)amino)prop-1-yn-1-yl)-3,4-difluorophenyl)-amino)-5-(trifluoromethyl)benzoic acid 37b

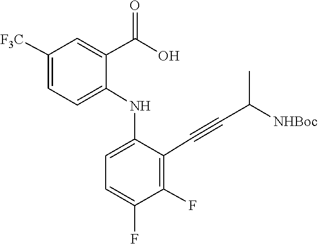 [SMILES: CC(C#Cc1c(Nc2ccc(C(F)(F)F)cc2C(=O)O)ccc(F)c1F)NC(=O)OC(C)(C)C]

Following the procedure outlined in Example 28, Step B, stirring the reaction mixture at 50° C. for 3 h, 2-((2-(3-((tert-butoxycarbonyl)amino)prop-1-yn-1-yl)-3,4-difluorophenyl)amino)-5-(trifluoromethyl) benzoic acid 37b (802 mg, 100% yield) was prepared as a yellow solid. HPLC/MS 1.37 min (A), [M-^{t}Bu]+415.1. ¹H NMR (DMSO-d₆, 400 MHz) δ 13.4-14.1 (m, 1H), 10.30 (s, 1H), 8.17 (d, 1H, J=1.5 Hz), 7.72 (br d, 1H, J=2.4 Hz), 7.50 (d, 1H, J=10.3 Hz), 7.37 (br d, 2H, J=9.3 Hz), 7.2-7.3 (m, 1H), 3.8-4.2 (m, 2H), 1.36 (s, 9H).

### Step C: 2-((2-(3-((tert-Butoxycarbonyl)amino)propyl)-3,4-difluorophenyl)amino)-5-(trifluoromethyl) benzoic acid 37c

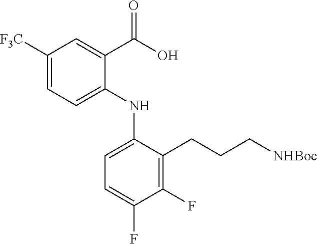 [SMILES: CC(C)(C)OC(=O)NCCCc1c(Nc2ccc(C(F)(F)F)cc2C(=O)O)ccc(F)c1F]

Following the procedure outlined in Example 28, Step C, replacing EtOH with EtOAc and stirring the reaction mixture at room temperature for 23 h, tert-butyl (3-(2-((5-((2-bromo-6-methoxypyridin-3-yl)carbamoyl)-2-(trifluoromethyl)pyridin-3-yl)amino)-5-fluorophenyl)propyl)carbamate 37c (769 mg, 93% yield) was prepared as an off-white foam. HPLC/MS 1.35 min (A), [M-Boc]+375.1. ¹H NMR (DMSO-d₆, 400 MHz) δ 13.4-13.9 (m, 1H), 9.89 (br s, 1H), 8.14 (d, 1H, J=2.0 Hz), 7.61 (dd, 1H, J=2.2, 9.0 Hz), 7.37 (d, 1H, J=9.8 Hz), 7.2-7.3 (m, 1H), 6.7-6.8 (m, 2H), 3.18 (s, 1H), 2.90 (br d, 2H, J=6.4 Hz), 2.59 (br t, 2H, J=7.1 Hz), 1.5-1.7 (m, 2H), 1.33 (s, 9H).

### Step D: tert-Butyl (3-(6-((2-((2-bromo-6-methoxypyridin-3-yl)carbamoyl)-4-(trifluoro-methyl)phenyl) amino)-2,3-difluorophenyl)propyl)carbamate 37d

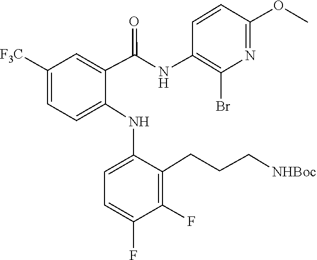 [SMILES: COc1ccc(NC(=O)c2cc(C(F)(F)F)ccc2Nc2ccc(F)c(F)c2CCCNC(=O)OC(C)(C)C)c(Br)n1]

Following the procedure outlined in Example 28, Step D, replacing DIEA with TEA and stirring the reaction mixture at room temperature for 21 h, then charging the reaction with additional 2-bromo-6-methoxypyridin-3-amine, HATU, TEA, and cat. DMAP and stirring at room temperature for 48 h, tert-butyl (3-(6-((2-((2-bromo-6-methoxypyridin-3-yl)carbamoyl)-4-(trifluoromethyl)phenyl)amino)-2,3-difluorophenyl)propyl)carbamate 37d (579 mg, 41% yield) was prepared as a red foam. HPLC/MS 1.54 min (A), [M-Boc]⁺ 560.9, in 74% purity.

### Step E: tert-Butyl (3-(6-(3-(2-bromo-6-methoxypyridin-3-yl)-4-oxo-6-(trifluoromethyl)-phenyl)amino)-3,4-dihydroquinazolin-1(2H)-yl)-2,3-difluorophenyl)propyl)carbamate 37e

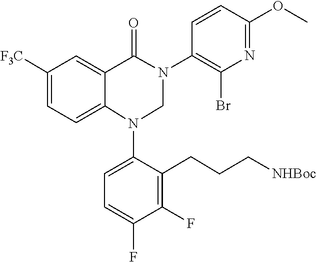 [SMILES: COc1ccc(N2CN(c3ccc(F)c(F)c3CCCNC(=O)OC(C)(C)C)c3ccc(C(F)(F)F)cc3C2=O)c(Br)n1]

Following the procedure outlined in Example 28, Step E, stirring the reaction mixture at 90° C. for 18 h, tert-butyl (3-(6-((2-((2-bromo-6-methoxypyridin-3-yl)carbamoyl)-4-(trifluoromethyl)phenyl)amino)-2,3-difluorophenyl)propyl)carbamate 37e (179 mg, 33% yield) was prepared as a white solid. HPLC/MS 1.44 min (A), [M+H]⁺ 616.9. ¹H NMR (DMSO-d₆, 400 MHz) δ 8.10 (br d, 1H, J=3.9 Hz), 7.87 (br d, 1H, J=8.8 Hz), 7.67 (br d, 1H, J=7.8 Hz), 7.48 (br s, 1H), 7.31 (br d, 1H, J=2.9 Hz), 6.9-7.0 (m, 1H), 6.7-6.9 (m, 1H), 6.3-6.6 (m, 1H), 5.63 (br d, 1H, J=9.3 Hz), 4.7-5.2 (m, 1H), 3.89 (s, 3H), 2.6-3.0 (m, 4H), 1.5-1.8 (m, 2H), 1.31 (s, 9H).

### Step F: 1-(2-(3-Aminopropyl)-3,4-difluorophenyl)-3-(2-bromo-6-methoxypyridin-3-yl)-6-(trifluoromethyl)-2,3-dihydroquinazolin-4(1H)-yl)-one, hydrochloride salt 37f

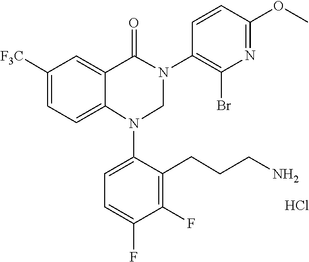 [SMILES: COc1ccc(N2CN(c3ccc(F)c(F)c3CCCN)c3ccc(C(F)(F)F)cc3C2=O)c(Br)n1 ; Cl]

Following the procedure outlined in Example 28, Step F, stirring the reaction mixture at room temperature for 41 h, 1-(2-(3-aminopropyl)-3,4-difluorophenyl)-3-(2-bromo-6-methoxypyridin-3-yl)-6-(trifluoro methyl)-2,3-dihydroquinazolin-4(1H)-yl)-one, hydrochloride salt 37f (179 mg, 100% yield) was prepared as a white solid. HPLC/MS 0.88 min (A), [M+H]⁺ 572.9, in 85% purity.

### Step G: 8-Fluoro-15-methoxy-2-(trifluoromethyl)-10,11,12,13-tetrahydro-19H-5,18-methanodibenzo[f,i]pyrido[2,3,-b][1,4,8]triazacyclotridecine-19-one 372

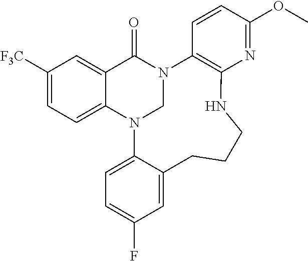 [SMILES: COc1ccc2c(n1)NCCCc1cc(F)ccc1N1CN2C(=O)c2cc(C(F)(F)F)ccc21]

Following the procedure outlined in Example 28, Step G, stirring the reaction mixture at 90° C. for 3 h, 8-fluoro-15-methoxy-2-(trifluoromethyl)-10,11,12,13-tetrahydro-19H-5,18-methanodibenzo[f,i]pyrido [2,3,-b][1,4,8]triazacyclo tridecine-19-one 37g (51 mg, 42% yield) was prepared as an off-white solid. HPLC/MS 1.31 min (A), [M+H]⁺ 491.1. ¹H NMR ((DMSO-d₆, 400 MHz) 8.14 (d, 1H, J=2.4 Hz), 7.67 (dd, 1H, J=2.2, 9.0 Hz), 7.4-7.5 (m, 2H), 7.3-7.4 (m, 1H), 6.5-6.6 (m, 1H), 6.11 (d, 1H, J=8.3 Hz), 6.0-6.1 (m, 1H), 5.46 (d, 1H, J=11.2 Hz), 4.4-4.5 (m, 1H), 3.78 (s, 3H), 3.6-3.7 (m, 1H), 3.0-3.1 (m, 1H), 2.82 (br s, 2H), 1.9-2.2 (m, 1H), 1.3-1.5 (m, 1H).

### Step H: 8,9-Difluoro-2-(trifluoromethyl)-10,11,12,13-tetrahydro-19H-5,18-methano-dibenzo[f,i]dipyrido [2,3,-b][1,4,8]triazacyclotridecine-15,19(14H)-dione

### Example 37

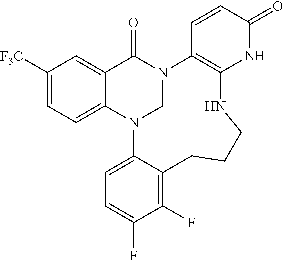 [SMILES: O=C1c2cc(C(F)(F)F)ccc2N2CN1c1ccc(=O)[nH]c1NCCCc1c2ccc(F)c1F]

Following the procedure outlined in Example 27, Step F, using 4N HCl in iPrOH and stirring the reaction mixture at 90° C. for 120 h, 8,9-difluoro-2-(trifluoromethyl)-10,11,12,13-tetrahydro-19H-5,18-methanodibenzo[f,i]dipyrido[2,3,-b][1,4,8]triazacyclotridecine-15,19(14H)-dione (20 mg, 43% yield) was prepared as a white solid. HPLC/MS 1.01 min (A), [M+H]⁺ 477.1. ¹H NMR (CD₃OD, 400 MHz) δ 8.29 (d, 1H, J=1.5 Hz), 7.64 (dd, 1H, J=1.7, 8.6 Hz), 7.49 (d, 1H, J=8.8 Hz), 7.2-7.4 (m, 2H), 6.59 (d, 1H, J=8.8 Hz), 5.97 (d, 1H, J=9.3 Hz), 5.53 (d, 1H, J=11.2 Hz), 4.65 (d, 1H, J=10.8 Hz), 3.4-3.6 (m, 1H), 3,21 (dt, 1H, J=4.4, 9.3 Hz), 2.89 (br d, 1H, J=13.7 Hz), 2.74 (br d, 1H, J=10.8 Hz), 2.1-2.3 (m, 1H), 1.52 (dt, 1H, J=5.1, 8.9 Hz).

### Example 38

### 8,9-Diluoro-2-(trifluoromethyl)-10,11,12,13-tetrahydro-19H-5,18-methanodibenzo[i]dipyrido[2,3,-b:4′,3′-f][1,4,8]triazacyclotridecine-15,19(14H)-dione

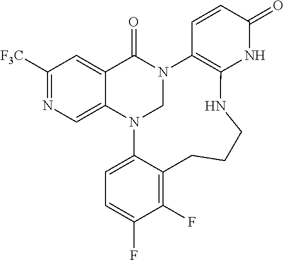 [SMILES: O=C1c2cc(C(F)(F)F)ncc2N2CN1c1ccc(=O)[nH]c1NCCCc1c2ccc(F)c1F]

### Step A: Methyl 5-((2-(3-((tert-butoxycarbonyl)amino)propyl)-3,4-difluorophenyl)-amino)-2-(trifluoromethyl)isonicotinate 38a

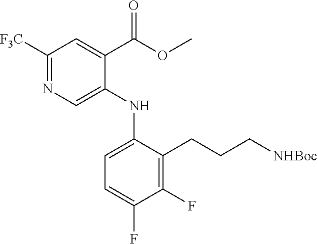 [SMILES: COC(=O)c1cc(C(F)(F)F)ncc1Nc1ccc(F)c(F)c1CCCNC(=O)OC(C)(C)C]

Following the procedure outlined in Example 28, Step A, substituting methyl 2-bromo-4-(trifluoromethyl)benzoate with methyl 5-bromo-2-(trifluoromethyl)isonicotinate and Int-3e with Int-3i, and stirring the reaction mixture at 80° C. for 6 h, methyl 5-((2-(3-((tert-butoxycarbonyl)amino)propyl)-3,4-difluorophenyl)amino)-2-(trifluoromethyl)isonicotinate 38a (1.13 g, 88% yield) was prepared as a yellow solid. HPLC/MS 1.38 min (B), [M+H]⁺ 490.1. ¹H NMR (CDCl₃, 400 MHz) δ 9.34 (br s, 1H), 8.28 (s, 1H), 8.11 (s, 1H), 7.10 (br s, 2H), 4.68 (br s, 1H), 4.02 (s, 3H), 3.09-3,24 (m, 2H), 2.72 (br t, J=7.1 Hz, 2H), 1.69-1.83 (m, 2H), 1.40 (s, 9H).

### Step B: 5-((2-(3-((tert-Butoxycarbonyl)amino)propyl)-3,4-difluorophenyl)amino)-2-(trifluoromethyl) isonicotinate 38b

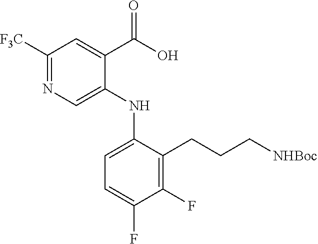 [SMILES: CC(C)(C)OC(=O)NCCCc1c(Nc2cnc(C(F)(F)F)cc2C(=O)O)ccc(F)c1F]

Following the procedure outlined in Example 28, Step B, using a THF/MeOH/H₂O mixture and stirring the reaction mixture at room temperature for 2 h, 5-((2-(3-((tert-butoxycarbonyl)amino)propyl)-3,4-difluorophenyl)amino)-2-(trifluoromethyl)isonicotonic acid 38b (1.32 g, 97% yield) was prepared as a yellow solid. HPLC/MS 0.90 min (B), [M+H]⁺ 476.0. ¹H NMR (CD₃OD, 400 MHz) δ 8.15 (s, 1H), 8.14 (s, 1H), 7.18-7.30 (m, 2H), 3.07 (br t, J=6.6 Hz, 2H), 2.63-2.82 (m, 2H), 1.54-1.80 (m, 2H), 1.39 (s, 9H).

### Step C: tert-Butyl (3-(6-((4-((2-bromo-6-methoxypyridin-3-yl)carbamoyl)-6-(trifluoromethyl)pyridin-3-yl)amino)-2,3-difluorophenyl)propyl)carbamate 38c

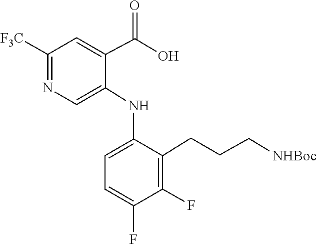 [SMILES: CC(C)(C)OC(=O)NCCCc1c(Nc2cnc(C(F)(F)F)cc2C(=O)O)ccc(F)c1F]

Following the procedure outlined in Example 28, Step D, replacing DMF with MeCN and stirring the reaction mixture at room temperature for 3 h, followed by additional equivalents of HATU and DIEA added and stirring continued at room temperature for 1 h, tert-butyl (3-(6-((4-((2-bromo-6-methoxypyridin-3-yl)carbamoyl)-6-(trifluoromethyl)pyridin-3-yl)amino)-2,3-difluorophenyl)propyl)carbamate 38c (1.12 g, 60% yield) was prepared as an orange solid. HPLC/MS 1.46 min (B), [M+H]⁺ 660.0, 662.0. ¹H NMR (CDCl₃, 400 MHz)₆-9.39 (s, 1H), 8.47 (br s, 1H), 8.37 (d, J=8.8 Hz, 1H), 8.34 (s, 1H), 7.83 (s, 1H), 7.04-7.13 (m, 2H), 6.81 (d, J=8.8 Hz, 1H), 3.97 (s, 3H), 3.16 (br d, J=5.9 Hz, 2H), 2.73 (br t, J=7.6 Hz, 2H), 1.69-1.78 (m, 2H), 1.36-1.41 (m, 9H).

### Step D: tert-Butyl (3-(6-(3-(2-bromo-6-methoxypyridin-3-yl)-4-oxo-6-(trifluoromethyl)-3,4-dihydro pyrido[3,4-d]pyrimidin-1(2H)-yl)-2,3-difluorophenyl)propyl)carbamate 38d

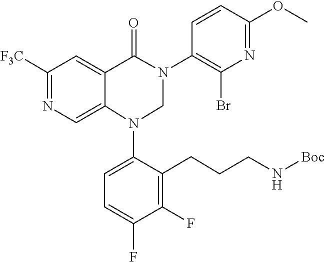 [SMILES: COc1ccc(N2CN(c3ccc(F)c(F)c3CCCNC(=O)OC(C)(C)C)c3cnc(C(F)(F)F)cc3C2=O)c(Br)n1]

Following the procedure outlined in Example 28, Step E, stirring the reaction mixture at room temperature for 30 min followed by heating at 90° C. for 4 h, tert-butyl (3-(6-(3-(2-bromo-6-methoxypyridin-3-yl)-4-oxo-6-(trifluoromethyl)-3,4-dihydropyrido[3,4-d]pyrimidin-1(2H)-yl)-2,3-difluorophenyl)propyl)carbamate 38d (49 mg, 48% yield) was prepared. HPLC/MS 1.39 min (B), [M+H]⁺ 672.1, 674.0. ¹H NMR (CDCl₃, 400 MHz) δ 8.29 (s, 1H), 7.86 (s, 1H), 7.44-7.71 (m, 1H), 7.03 (br dd, J=3.4, 2.0 Hz, 1H), 6.81 (br d, J=8.3 Hz, 1H), 5.23-5.33 (m, 1H), 5.23-5.41 (m, 1H), 4.98 (br d, J=9.8 Hz, 1H), 4.42-4.64 (m, 1H), 3.98 (s, 3H), 3.15 (br d, J=6.4 Hz, 2H), 2.84-3.00 (m, 1H), 2.63-3.00 (m, 1H), 1.71-1.83 (m, 2H), 1.40 (s, 9H).

### Step E: 1-(2-(3-Aminopropyl)-3,4-difluorophenyl)-3-(2-bromo-6-methoxypyridin-3-yl)-6-(trifluoromethyl)-2,3-dihydropyrido[3,4-d]pyrimidin-4(11H)-one, hydrochloride salt 38e

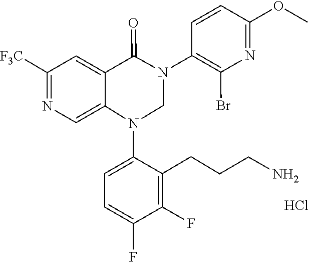 [SMILES: COc1ccc(N2CN(c3ccc(F)c(F)c3CCCN)c3cnc(C(F)(F)F)cc3C2=O)c(Br)n1 ; Cl]

Following the procedure outlined in Example 28, Step F, dissolving the intermediate in DCM to which was added 4N HCl in dioxane and stirring the reaction mixture at room temperature for 1 h, 1-(2-(3-aminopropyl)-3,4-difluorophenyl)-3-(2-bromo-6-methoxypyridin-3-yl)-6-(trifluoromethyl)-2,3-dihydro pyrido[3,4-d]pyrimidin-4(1H)-one, hydrochloride salt 38e (518 mg, 97% yield) was prepared as a yellow solid. HPLC/MS 1.13 min (B), [M+H]⁺ 572.0, 573.9.

### Step F: 8,9-Difluoro-15-methoxy-2-(trifluoromethyl)-10,11,12,13-tetrahydro-19H-5,18-methanodibenzo [i]dipyrido[2,3,-b:4′,3′-f][1,4,8]triazacyclotridecine-19-one 38f

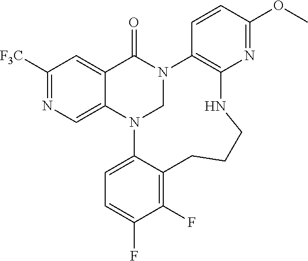 [SMILES: COc1ccc2c(n1)NCCCc1c(ccc(F)c1F)N1CN2C(=O)c2cc(C(F)(F)F)ncc21]

Following the procedure outlined in Example 28, Step G, stirring the reaction mixture at 50° C. for 3 h, followed by 70° C. for 1 h, 8,9-difluoro-15-methoxy-2-(trifluoromethyl)-10,11,12,13-tetrahydro-19H-5,18-methanodibenzo[i]dipyrido[2,3,-b:4′,3′-f][1,4,8]triazacyclotridecine-19-one 38f (32 mg, 12% yield) was prepared as a light yellow solid. HPLC/MS 1.26 min (B), [M+H]⁺ 492.1. ¹H NMR (CD₃OD, 400 MHz) δ 8.82 (s, 1H), 8.53 (s, 1H), 7.92 (br d, J=8.31 Hz, 1H), 7.65-7.72 (m, 1H), 6.91 (d, J=8.3 Hz, 1H), 7.31 (br d, J=9.3 Hz, 1H), 6.91 (d, J=8.3 Hz, 1H), 3.96 (s, 3H), 3.75 (d, J=5.4 Hz, 1H), 3.66-3.69 (m, 1H), 3.61 (d, J=5.4 Hz, 1H), 3.37 (s, 1H), 3.02 (br t, J=6.9 Hz, 2H), 2.88 (br t, J=6.9 Hz, 2H), 1.97 (br s, 2H).

### Step G: 8,9-Difluoro-2-(trifluoromethyl)-10,11,12,13-tetrahydro-19H-5,18-methanodibenzo[i]dipyrido [2,3,-b:4′,3′-f][1,4,8]triazacyclotridecine-15,19(14H)-dione

### Example 38

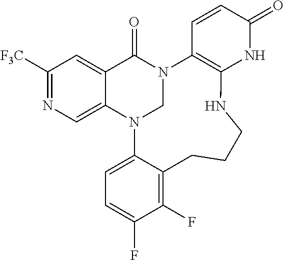 [SMILES: O=C1c2cc(C(F)(F)F)ncc2N2CN1c1ccc(=O)[nH]c1NCCCc1c2ccc(F)c1F]

Following the procedure outlined in Example 27, Step F, stirring the reaction mixture at 90° C. for 16 h, 8,9-difluoro-2-(trifluoromethyl)-10,11,12,13-tetrahydro-19H-5,18-methanodibenzo[i]dipyrido[2,3,-b:4′,3′-f][1,4,8]triazacyclotridecine-15,19(14H)-dione (10 mg, 32% yield) was prepared as an off-white solid. HPLC/MS 0.93 min (B), [M+H]⁺ 478.0. ¹H NMR (CD₃OD, 400 MHz) δ 8.28 (s, 1H), 7.97 (s, 1H), 7.35 (d, J=9.3 Hz, 1H), 7.18-7.28 (m, 1H), 7.01-7.17 (m, 1H), 6.04 (br d, J=9.3 Hz, 1H), 5.50 (d, J=11.3 Hz, 1H), 4.79-5.03 (m, 1H), 4.47 (d, J=10.8 Hz, 1H), 3.45-3.67 (m, 1H), 3.25-3.43 (m, 1H), 2.86-3.05 (m, 1H), 2.46-2.60 (m, 1H), 2.23-2.39 (m, 1H).

### Example 39

### 7,8-Difluoro-2-(trifluoromethyl)-10,11,12,13-tetrahydro-19H-5,18-methanodibenzo[f,i]dipyrido[2,3,-b][1,4,8]triazacyclotridecine-15,19(14H)-dione

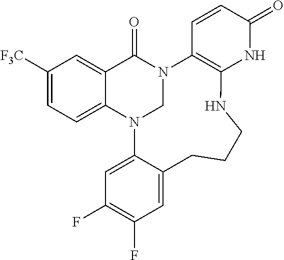 [SMILES: O=C1c2cc(C(F)(F)F)ccc2N2CN1c1ccc(=O)[nH]c1NCCCc1cc(F)c(F)cc12]

### Step A: Methyl 2-((2-(3-((tert-butoxycarbonyl)(6-methoxy-3-nitropyridin-2-yl)amino)-prop-1-yn-1-yl)-4,5-difluorophenyl)amino)-5-(trifluoromethyl)benzoate 39a

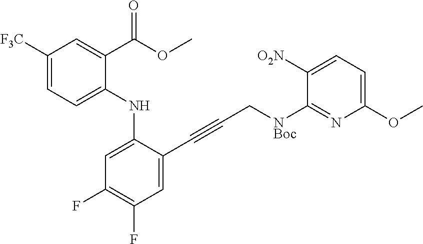 [SMILES: COC(=O)c1cc(C(F)(F)F)ccc1Nc1cc(F)c(F)cc1C#CCN(C(=O)OC(C)(C)C)c1nc(OC)ccc1[N+](=O)[O-]]

To a solution of methyl 2-bromo-5-(trifluoromethyl)benzoate (1.35 g, 4.77 mmol) and tert-butyl (3-(2-amino-4,5-difluorophenyl)prop-2-yn-1-yl)(6-methoxy-3-nitropyridin-2-yl)carbamate (2.93 g, 5.72 mmol), in N₂ purged 1,4-dioxane (35 mL), was added palladium(II) acetate (0.054 g, 0.238 mmol), BINAP (0.297 g, 0.477 mmol), the reaction mixture purged with N₂, followed by Cs₂CO₃ (2.176 g, 6.68 mmol), and the reaction mixture was heated at 80° C. for 24 h. Additional methyl 2-bromo-5-(trifluoromethyl)benzoate (1.35 g, 4.77 mmol) was added and the reaction mixture stirred at 80° C. for 23 h. The reaction mixture was cooled to ambient temperature, additional palladium(II) acetate (0.054 g, 0.238 mmol), BINAP (0.297 g, 0.477 mmol) and cesium carbonate (2.176 g, 6.68 mmol) were added and the reaction mixture was stirred for 25 h. The reaction mixture was cooled to ambient temperature, diluted with EtOAc, washed with H₂O (2×), brine, dried over Na₂SO₄, filtered and the solvent evaporated under reduced pressure. The crude residue was dissolved in DCM, absorbed onto a silica gel packed precolumn and purified by flash column chromatogarphy (330 g) eluting with a 100% heptane to 30% EtOAc-heptanes gradient. Product fractions were combined and evaporated under reduced pressure to afford methyl 2-((2-(3-((tert-butoxycarbonyl)(6-methoxy-3-nitropyridin-2-yl)amino)prop-1-yn-1-yl)-4,5-difluorophenyl)amino)-5-(trifluoromethyl)benzoate 39a (1.25 g, 37% yield) as a light yellow foam. HPLC/MS 1.63 min (A), [M+H]⁺ 637.1. ¹H NMR (DMSO-d₆, 400 MHz) δ 9.6-9.9 (m, 1H), 8.27 (d, 1H, J=8.8 Hz), 8.07 (d, 1H, J=1.5 Hz), 7.5-7.7 (m, 3H), 7.25 (d, 1H, J=8.8 Hz), 6.7 (d, 1H, J=8.8 Hz), 4.89 (s, 2H), 3.84 (s, 3H), 3.76 (s, 3H), 1.2-1.4 (m, 12H).

### Step B: 2-((2-(3-((tert-Butoxycarbonyl)(2-methoxy-5-nitropyridin-4-yl)amino)prop-1-yn-1-yl)-4,5-difluoro phenyl)amino)-5-(trifluoromethyl)benzoic acid 39b

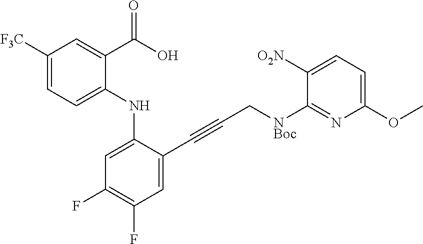 [SMILES: COc1ccc([N+](=O)[O-])c(N(CC#Cc2cc(F)c(F)cc2Nc2ccc(C(F)(F)F)cc2C(=O)O)C(=O)OC(C)(C)C)n1]

Following the procedure outlined in Example 27, Step B, stirring the reaction mixture at room temperature for 72 h, 2-((2-(3-((tert-butoxycarbonyl)(2-methoxy-5-nitropyridin-4-yl)amino)prop-1-yn-1-yl)-4,5-difluorophenyl)amino)-5-(trifluoromethyl)benzoic acid 39b (749 mg, 59% yield) was prepared as a white foam. HPLC/MS 1.52 min (A), [M+H]⁺ 423.0. ¹H NMR (DMSO-d₆, 400 MHz) δ 13.64 (br s, 1H), 10.11 (br s, 1H), 8.28 (d, 1H, J=8.8 Hz), 8.11 (d, 1H, J=2.0 Hz), 7.5-7.8 (m, 3H), 7.25 (d, 1H, J=8.8 Hz), 6.78 (d, 1H, J=9.3 Hz), 4.89 (s, 2H), 3.87 (s, 3H), 3.2-3.5 (m, 1H), 1.30 (br s, 9H).

### Step C: 2-((2-(3-((3-Amino-6-methoxypyridin-2-yl)(tert-butoxycarbonyl)amino)propyl)-4,5-difluoro phenyl)amino)-5-(trifluoromethyl)benzoic acid 39c

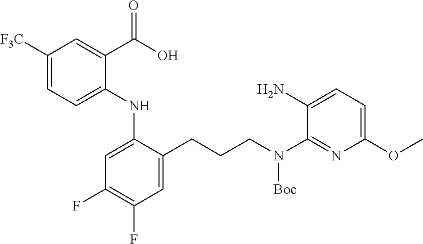 [SMILES: COc1ccc(N)c(N(CCCc2cc(F)c(F)cc2Nc2ccc(C(F)(F)F)cc2C(=O)O)C(=O)OC(C)(C)C)n1]

Following the procedure outlined in Example 27, Step C, stirring the reaction mixture at room temperature under H₂ atmosphere for 4 h, 2-((2-(3-((3-amino-6-methoxypyridin-2-yl)(tert-butoxycarbonyl)amino) propyl)-4,5-difluorophenyl)amino)-5-(trifluoromethyl)benzoic acid 39c (655 mg, 87% yield) was prepared as a beige foam. HPLC/MS 1.34 min (A), [M+H]⁺ 597.5. ¹H NMR (DMSO-d₆, 400 MHz) δ 9.6-10.0 (m, 1H), 8.12 (d, 1H, J=2.0 Hz), 7.5-7.6 (m, 1H), 7.4-7.5 (m, 1H), 7.34 (s, 1H), 7.14 (br s, 1H), 6.77 (d, 1H, J=8.8 Hz), 6.51 (br d, 1H, J=8.3 Hz), 3.61 (s, 3H), 3.4-3.6 (m, 2H), 2.5-2.6 (m, 2H), 1.6-1.8 (m, 2H), 1.29 (s, 10H).

### Step D: tert-Butyl 7,8-difluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 39d

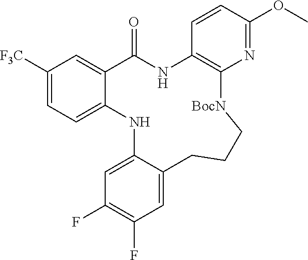 [SMILES: COc1ccc2c(n1)N(C(=O)OC(C)(C)C)CCCc1cc(F)c(F)cc1Nc1ccc(C(F)(F)F)cc1C(=O)N2]

Following the procedure outlined in Example 27, Step D, and stirring the reaction mixture at room temperature for 72 h, tert-butyl 7,8-difluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 39d (420 mg, 65% yield) was prepared as a yellow foam. HPLC/MS 1.52 min (A), [M+H]⁺ 579.5. ¹H NMR (DMSO-d₆, 400 MHz) δ 8.7-9.1 (m, 1H), 8.44 (br s, 1H), 8.01 (br s, 1H), 7.62 (br d, 1H, J=8.8 Hz), 7.52 (dd, 1H, J=9.0, 11.5 Hz), 7.37 (br s, 1H), 6.88 (d, 1H, J=8.8 Hz), 6.75 (d, 1H, J=8.8 Hz), 3.9-4.1 (m, 1H), 3.83 (s, 3H), 1.4-1.7 (m, 2H), 1.0-1.4 (m, 12H).

### Step E: tert-Butyl 7,8-difluoro-15-methoxy-19-oxo-2-(trifluoromethyl)-11,12-dihydro-19H-5,18-methano dibenzo[f,i]pyrido[2,3-b][1,4,8]triazacyclotridecine-13(10H)-carboxylate 39e

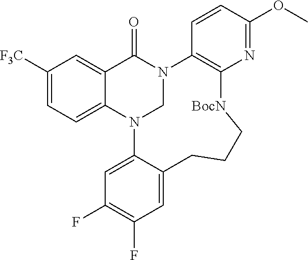 [SMILES: COc1ccc2c(n1)N(C(=O)OC(C)(C)C)CCCc1cc(F)c(F)cc1N1CN2C(=O)c2cc(C(F)(F)F)ccc21]

Following the procedure outlined in Example 27, Step E, and stirring the reaction mixture at 90° C. for 48 h, tert-butyl 7,8-difluoro-15-methoxy-19-oxo-2-(trifluoromethyl)-11,12-dihydro-19H-5,18-methano dibenzo[f,i]pyrido[2,3-b][1,4,8]triazacyclotridecine-13(10H)-carboxylate 39e (336 mg, 77% yield) was prepared as a white solid. HPLC/MS 1.43 min (A), [M+H]⁺ 591.1. ¹H NMR (DMSO-d₆, 400 MHz) δ 8.16 (d, 1H, J=2.0 Hz), 7.9-7.9 (m, 1H), 7.6-7.7 (m, 3H), 6.91 (d, 1H, J=8.8 Hz), 6.6-6.7 (m, 1H), 5.3-5.4 (m, 1H), 4.4-4.7 (m, 1H), 3.85 (s, 3H), 3.6-3.7 (m, 1H), 3.3-3.5 (m, 2H), 2.5-2.6 (m, 1H), 1.8-2.0 (m, 1H), 1.4-1.6 (m, 1H), 1.25 (br s, 1H), 1.08 (s, 10H).

### Step F: 7,8-Difluoro-2-(trifluoromethyl)-10,11,12,13-tetrahydro-19H-5,18-methano-dibenzo[f,i]pyrido [2,3-b][1,4,8]triazacyclotridecine-15,19(14H)-dione Example 39

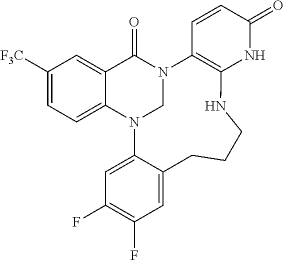 [SMILES: O=C1c2cc(C(F)(F)F)ccc2N2CN1c1ccc(=O)[nH]c1NCCCc1cc(F)c(F)cc12]

Following the procedure outlined in Example 27, Step F, stirring the reaction mixture at 90° C. for 72 h, 7,8-difluoro-2-(trifluoromethyl)-10,11,12,13-tetrahydro-19H-5,18-methanodibenzo[f,i]pyrido[2,3-b][1,4,8]triazacyclotridecine-15,19(14H)-dione (143 mg, 54% yield) was prepared as a white solid. HPLC/MS 1.02 min (A), [M+H]⁺ 477.1. ¹H NMR (DMSO-d₆, 400 MHz) δ 10.3-11.1 (m, 1H), 8.12 (d, 1H, J=2.0 Hz), 7.5-7.7 (m, 3H), 7.33 (d, 1H, J=8.8 Hz), 6.50 (d, 1H, J=8.8 Hz), 5.6-5.9 (m, 2H), 5.46 (d, 1H, J=11.2 Hz), 4.54 (d, 1H, J=11.2 Hz), 3.2-3.4 (m, 3H), 2.9-3.1 (m, 1H), 2.6-2.8 (m, 1H), 1.8-2.2 (m, 1H), 1.2-1.6 (m, 1H).

### Example 40

### 8-Fluoro-2-(trifluoromethyl)-10,11,12,13-tetrahydro-19H-5,18-methanobenzo[l]dipyrido[3,4-b:3′,2′-f][1,5]diazocyclotridecine-15,19(14H)-dione

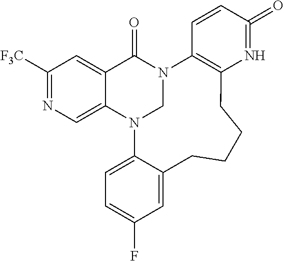

### Step A: Methyl 5-((2-(but-3-en-1-yl)-4-fluorophenyl)amino)-2-(trifluoromethyl)-isonicotinate 40a

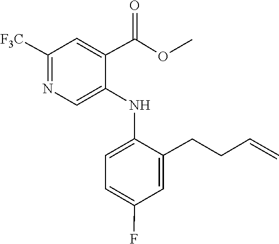 [SMILES: C=CCCc1cc(F)ccc1Nc1cnc(C(F)(F)F)cc1C(=O)OC]

Following the procedure outlined in Example 7, Step A, substituting ethyl 2-bromo-5-(trifluoromethyl)benzoate with methyl 5-bromo-2-(trifluoromethyl)isonicotinate, Pd(OAc)₂ with Pd₂(dba)₃, toluene with 1,4-dioxane, and stirring the reaction mixture at 100° C. for 4 h, methyl 5-((2-(but-3-en-1-yl)-4-fluorophenyl)amino)-2-(trifluoromethyl)isonicotinate 40a (4.84 g, 59% yield) was prepared as a yellow oil. HPLC/MS 1.42 min (B), [M+H]⁺ 369.1. ¹H NMR (CDCl₃, 400 MHz) δ 9.20 (s, 1H), 8.16 (s, 1H), 8.07 (s, 1H), 7.21-7.26 (m, 1H), 6.92-7.10 (m, 2H), 5.69-5.84 (m, 1H), 4.91-5.04 (m, 2H), 3.99 (s, 3H), 2.62-2.75 (m, 2H), 2.28-2.38 (m, 2H).

### Step B: 5-((2-(But-3-en-1-yl)-4-fluorophenyl)amino)-2-(trifluoromethyl)isonicotinic acid 40b

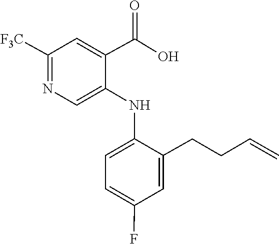 [SMILES: C=CCCc1cc(F)ccc1Nc1cnc(C(F)(F)F)cc1C(=O)O]

Following the procedure outlined in Example 7, Step B, stirring the reaction mixture in THF/MeOH/H₂O at room temperature for 5 h, 5-((2-(but-3-en-1-yl)-4-fluorophenyl)amino)-2-(trifluoromethyl)isonicotinic acid 40b (12.2 g, 96% yield) was prepared as a yellow solid. HPLC/MS 0.77 min (B), [M+H]⁺ 355.0. ¹H NMR (CD₃OD, 400 MHz) δ 8.12 (s, 1H), 8.07 (s, 1H), 7.37 (dd, J=8.6, 5.1 Hz, 1H), 7.17 (dd, J=9.3, 2.9 Hz, 1H), 7.05-7.11 (m, 1H), 5.72-5.88 (m, 1H), 4.90-4.98 (m, 2H), 2.72 (t, J=7.6 Hz, 2H), 2.29-2.37 (m, 2H).

### Step C: N-(2-Allyl-6-methoxypyridin-3-yl)-5-((2-(but-3-en-1-yl)-4-fluorophenyl)amino)-2-(trifluoromethyl) isonicotinamide 40c

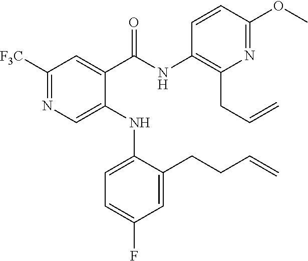 [SMILES: C=CCCc1cc(F)ccc1Nc1cnc(C(F)(F)F)cc1C(=O)Nc1ccc(OC)nc1CC=C]

Following the procedure outlined in Scheme 7, Step C, substituting DMF with MeCN and stirring the reaction mixture at room temperature for 5 h, N-(2-allyl-6-methoxypyridin-3-yl)-5-((2-(but-3-en-1-yl)-4-fluorophenyl)amino)-2-(trifluoromethyl)isonicotinamide 40c (5.20 g, 89% yield) was prepared as a yellow oil. HPLC/MS 1.51 min (B), [M+H]⁺ 501.3. ¹H NMR (CDCl₃, 400 MHz) δ 9.68 (s, 1H), 8.34-8.48 (m, 2H), 8.18 (br d, J=8.8 Hz, 1H), 7.89 (d, J=4.9 Hz, 1H) 7.40-7.49 (m, 1H), 7.25 (dd, J=9.3, 2.9 Hz, 1H), 7.15-7.21 (m, 1H), 6.92 (dd, J=8.6, 4.2 Hz, 1H), 6.25-6.37 (m, 1H), 5.93-6.04 (m, 1H), 5.42-5.56 (m, 2H), 5.11-5.22 (m, 2H), 4.16 (d, J=1.5 Hz, 3H), 3.80-3.87 (m, 2H), 2.90 (t, J=7.6 Hz, 2H), 2.54 (q, J=6.9 Hz, 2H).

### Step D: (Z)-1-(2-(But-3-en-1-yl)-4-fluorophenyl)-3-(6-methoxy-2-(prop-1-en-1-yl)pyridin-3-yl)-6-(trifluoro methyl)-2,3-dihydropyrido[3,4-d]pyrimidin-4(11H)-one 40d

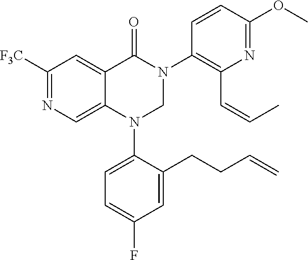 [SMILES: C=CCCc1cc(F)ccc1N1CN(c2ccc(OC)nc2/C=C\C)C(=O)c2cc(C(F)(F)F)ncc21]

Following the procedure outlined in Example 27, Step E, stirring the reaction mixture at 85° C. for 6 h, (Z)-1-(2-(but-3-en-1-yl)-4-fluorophenyl)-3-(6-methoxy-2-(prop-1-en-1-yl)pyridin-3-yl)-6-(trifluoromethyl)-2,3-dihydropyrido[3,4-d]pyrimidin-4(1H)-one 40d (1.66 g, 22% yield) was prepared as a yellow solid. HPLC/MS 1.50 min (B), [M+H]⁺ 513.2. ¹H NMR (CDCl₃, 400 MHz) δ 8.21-8.27 (m, 1H) 7.86 (s, 1H), 7.80 (s, 1H), 7.35-7.45 (m, 1H), 7.26-7.29 (m, 1H), 7.00-7.26 (m, 4H), 6.64 (d, J=8.8 Hz, 1H), 6.42 (br s, 1H), 5.68-5.79 (m, 1H), 5.37 (br d, J=9.3 Hz, 1H), 5.13 (br d, J=10.3 Hz, 1H), 4.89-5.05 (m, 3H), 4.66 (br d, J=9.3 Hz, 1H), 3.91-3.99 (m, 3H), 2.86 (s, 1H), 2.65-2.98 (m, 1H), 2.31-2.44 (m, 2H), 1.90 (br dd, J=12.0, 7.1 Hz, 3H).

### Step E: 8-Fluoro-15-methoxy-2-(trifluoromethyl)-10,11-dihydro-19H-5,18-methano-benzo[l]dipyrido[3,4-b:3′,2′-f][1,5]diazocyclotridecin-19-one 40e

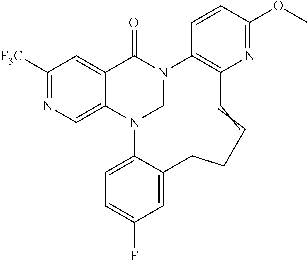 [SMILES: COc1ccc2c(n1)C=CCCc1cc(F)ccc1N1CN2C(=O)c2cc(C(F)(F)F)ncc21]

Following the procedure outlined in Example 7, Step E, stirring the reaction mixture at 80° C. for 18.5 h, 8-fluoro-15-methoxy-2-(trifluoromethyl)-10,11-dihydro-19H-5,18-methanobenzo[l]dipyrido[3,4-b:3′,2′-f][1,5]diazocyclotridecin-19-one 40e (44 mg, 3% yield) was prepared. HPLC/MS 1.28 min (B), [M+H]⁺ 471.2. ¹H NMR (CDCl₃, 400 MHz) δ 8.42 (s, 1H), 7.96 (s, 1H), 7.87 (d, J=8.8 Hz, 1H), 7.16-7.35 (m, 4H), 6.82 (d, J=8.8 Hz, 1H), 6.56 (dd, J=11.0, 3.7 Hz, 1H), 6.33 (dd, J=16.4, 1.7 Hz, 1H), 5.72 (d, J=10.3 Hz, 1H), 4.99 (d, J=10.8 Hz, 1H), 4.04 (s, 3H), 2.78-2.95 (m, 3H), 2.30-2.42 (m, 1H).

### Step F: 8-Fluoro-15-methoxy-2-(trifluoromethyl)-10,11,12,13-tetrahydro-19H-5,18-methanobenzo[l]dipyrido[3,4-b:3′,2′-f][1,5]diazocyclotridecin-19-one 40f

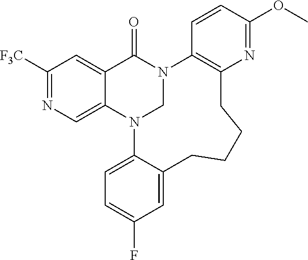 [SMILES: COc1ccc2c(n1)CCCCc1cc(F)ccc1N1CN2C(=O)c2cc(C(F)(F)F)ncc21]

Following the procedure outlined in Example 20, Step F, stirring the reaction mixture at 50° C. for 23.5 h, the reaction mixture was cooled to ambient temperature, additional Grubbs II catalyst M204 (7.94 mg, 9.35 μmol) and sodium borohydride (7.08 mg, 0.187 mmol) and 1 mL DCE:MeOH and the reaction mixture was heated at 50° C. for 5 h, 8-fluoro-15-methoxy-2-(trifluoromethyl)-10,11-dihydro-19H-5,18-methano benzo[l]dipyrido[3,4-b:3′,2′-f][1,5]diazocyclotridecin-19-one 40f (24 mg, 52% yield) was prepared. HPLC/MS 1.29 min (B), [M+H]⁺ 473.1. ¹H NMR (CDCl₃, 400 MHz) δ 8.27 (s, 1H), 7.88 (s, 1H), 7.41 (d, J=8.80 Hz, 1H), 7.18-7.25 (m, 1H), 7.13 (dd, J=9.3, 2.9 Hz, 1H), 7.04 (ddd, J=8.7, 7.7, 3.2 Hz, 1H), 6.64 (d, J=8.8 Hz, 1H), 5.60 (d, J=11.3 Hz, 1H), 4.60 (d, J=11.3 Hz, 1H), 3.90-3.95 (m, 3H), 2.87-3.02 (m, 2H), 2.47-2.58 (m, 1H), 2.35-2.46 (m, 1H), 1.67-1.91 (m, 3H), 1.29-1.41 (m, 2H).

### Step G: 8-Fluoro-2-(trifluoromethyl)-10,11,12,13-tetrahydro-19H-5,18 methanobenzo[l]dipyrido[3,4-b:3′,2′-f][1,5]diazocyclotridecin-15,19(14H)-dione

### Example 40

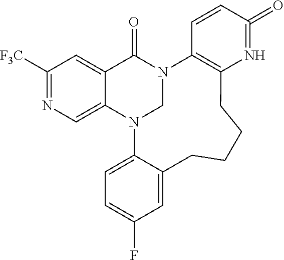

Following the procedure outlined in Example 7, Step G, stirring the reaction mixture at 95° C. for 72 h, 8-fluoro-2-(trifluoromethyl)-10,11,12,13-tetrahydro-19H-5,18-methanobenzo[l]-dipyrido[3,4-b:3′,2′-f][1,5]diazocyclotridecin-15,19(14H)-dione (6.0 mg, 42% yield) was prepared as a white solid. HPLC/MS 0.93 min (B), [M+H]⁺ 459.1. ¹H NMR (CDCl₃, 400 MHz) δ 8.27 (s, 1H), 7.92 (s, 1H), 7.37 (d, J=9.3 Hz, 1H), 7.25-7.28 (m, 1H), 7.17 (dd, J=8.8, 2.9 Hz, 1H), 7.06-7.12 (m, 1H), 6.50 (d, J=9.3 Hz, 1H), 5.58 (d, J=10.8 Hz, 1H), 4.62 (d, J=11.3 Hz, 1H), 2.73-2.98 (m, 2H), 2.50-2.67 (m, 1H), 2.37-2.50 (m, 1H), 1.73-1.96 (m, 2H), 1.38-1.54 (m, 1H), 1.28 (s, 1H).

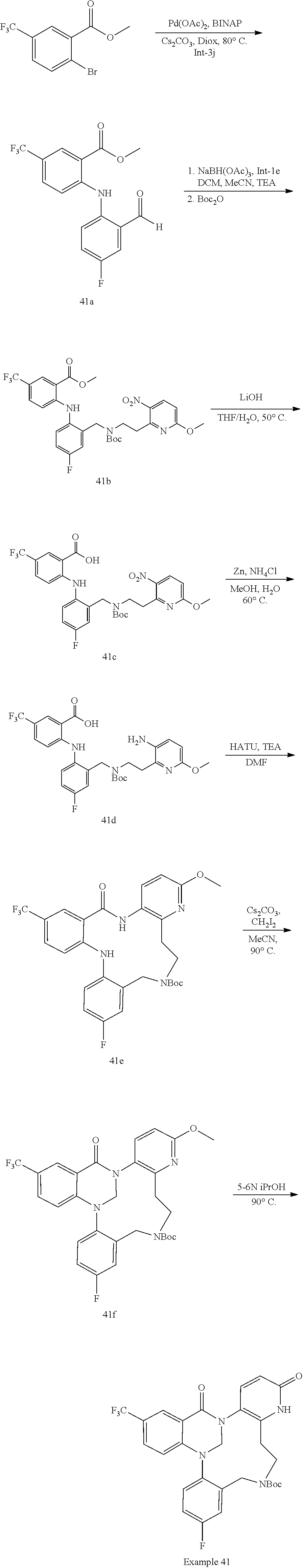

### Example 41

### 8-Fluoro-2-(trifluoromethyl)-10,11,12,13-tetrahydro-19H-5,18-methanodibenzo[c,f]pyrido[3,2-f][1,5,9]triazacyclotridecine-15,19(14H)-dione

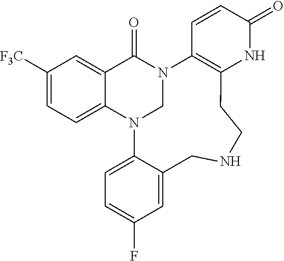 [SMILES: O=C1c2cc(C(F)(F)F)ccc2N2CN1c1ccc(=O)[nH]c1CCNCc1cc(F)ccc12]

### Step A: Methyl 2-((4-fluoro-2-formylphenyl)amino)-5-(trifluoromethyl)benzoate 41a

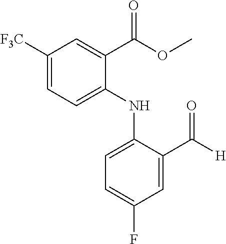 [SMILES: COC(=O)c1cc(C(F)(F)F)ccc1Nc1ccc(F)cc1C=O]

To a solution of methyl 2-bromo-5-(trifluoromethyl)benzoate (10.0 g, 35.3 mmol) and 2-amino-5-fluorobenzaldehyde (5.90 g, 42.4 mmol), in N₂ purged 1,4-dioxane (250 mL), was added palladium(II) acetate (0.397 g, 1.77 mmol), BINAP (2.20 g, 3.53 mmol) and Cs₂CO₃ (16.1 g, 49.5 mmol), and the reaction mixture was heated at 80° C. for 3 h. The reaction mixture was cooled to ambient temperature, the reaction mixture diluted with EtOAc, the organic phase washed with H₂O, brine, dried over Na₂SO₄, filtered and the solvent evaporated under reduced pressure. DCM was added to the crude residue, the solid filtered, rinsed with DCM, and dried under vacuo to afford methyl 2-((4-fluoro-2-formylphenyl)amino)-5-(trifluoromethyl)benzoate 41a (6.27 g, 50% yield) as a yellow solid. HPLC/MS 1.27 min (A), [M+H]⁺ 341.9. ¹H NMR (DMSO-d₆, 400 MHz) δ 10.83 (s, 1H), 10.00 (s, 1H), 8.1-8.2 (m, 1H), 7.78 (s, 2H), 7.6-7.7 (m, 1H), 7.5-7.6 (m, 1H), 7.48 (d, 1H, J=8.8 Hz), 3.93 (s, 3H). The filtrate was evaporated under reduced pressure, dissolved in DCM, absorbed onto a silica gel packed precolumn and purified by silica gel flash column chromatography (220 g) eluting with a 70% heptane-DCM to 100% DCM gradient. Product fractions were combined and evaporated under reduced pressure to afford a second batch of methyl 2-((4-fluoro-2-formylphenyl)amino)-5-(trifluoromethyl)benzoate 41a (4.91 g, 40% yield) as a yellow solid. HPLC/MS 1.27 min (A), [M+H]⁺ 341.9. ¹H NMR (DMSO-d₆, 400 MHz) δ 10.83 (s, 1H), 10.00 (s, 1H), 8.1-8.2 (m, 1H), 7.78 (s, 2H), 7.6-7.7 (m, 1H), 7.5-7.6 (m, 1H), 7.48 (d, 1H, J=8.8 Hz), 3.92 (s, 3H).

### Step B: Methyl 2-((2-(((tert-butoxycarbonyl)(2-(6-methoxy-3-nitropyridin-2-yl)ethyl)-amino)methyl)-4-fluorophenyl)amino)-5-(trifluoromethyl)benzoate 41b

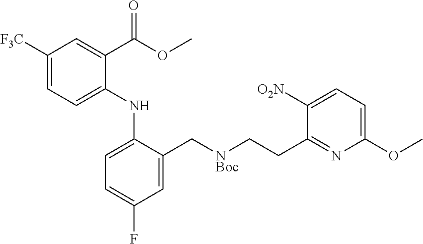 [SMILES: COC(=O)c1cc(C(F)(F)F)ccc1Nc1ccc(F)cc1CN(CCc1nc(OC)ccc1[N+](=O)[O-])C(=O)OC(C)(C)C]

To methyl 2-((4-fluoro-2-formylphenyl)amino)-5-(trifluoromethyl)benzoate (3.00 g, 8.79 mmol) was added 2-(6-methoxy-3-nitropyridin-2-yl)ethan-1-amine, bis-trifluoroacetate (5.58 g, 13.2 mmol), in 1,2-DCE (60 mL) and MeCN (30.0 mL), at room temperature, was added TEA (2.33 mL, 16.7 mmol) and the reaction mixture stirred at room temperature for 1 h. Sodium triacetoxyborohydride (4.66 g, 21.9 mmol) was added and the reaction mixture stirred at room temperature for 69 h. Boc-anhydride (9.59 g, 44.0 mmol) was added and the reaction mixture stirred at room temperature for 5 h. The reaction mixture was diluted with DCM, washed with H₂O, dried over Na₂SO₄, filtered and the solvent evaporated under reduced pressure. The crude oil was dissolved in DCM, preabsorbed onto a silica gel packed pre-column, and purified by silica gel flash chromatography (220 g) eluting with a 100% heptanes to 50% heptanes-EtOAc gradient. Product fractions were combined and evaporated under reduced pressure to afford methyl 2-((2-(((tert-butoxycarbonyl)(2-(6-methoxy-3-nitropyridin-2-yl)ethyl)amino)methyl)-4-fluoro phenyl)amino)-5-(trifluoromethyl)benzoate 41b (4.65 g, 82% yield) as a light yellow foam. HPLC/MS 1.66 min (A), [M+H]⁺ 623.1. ¹H NMR (DMSO-d₆, 400 MHz) δ 9.2-9.4 (m, 1H), 8.28 (br d, 1H, J=8.8 Hz), 8.10 (d, 1H, J=1.5 Hz), 7.5-7.6 (m, 1H), 7.34 (br d, 1H, J=5.4 Hz), 7.23 (br d, 1H, J=2.9 Hz), 7.0-7.2 (m, 1H), 6.7-6.9 (m, 1H), 6.4-6.7 (m, 1H), 4.32 (br s, 2H), 3.90 (s, 3H), 3.83 (s, 3H), 3.5-3.7 (m, 2H), 3.13 (br s, 2H), 1.18 (s, 10H).

### Step C: 2-((2-(((tert-Butoxycarbonyl)(2-(6-methoxy-3-nitropyridin-2-yl)ethyl)amino)-methyl)-4-fluoro phenyl)amino-5-(trifluoromethyl)benzoic acid 41c

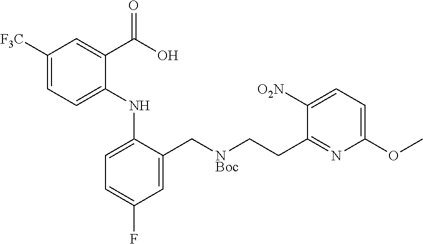 [SMILES: COc1ccc([N+](=O)[O-])c(CCN(Cc2cc(F)ccc2Nc2ccc(C(F)(F)F)cc2C(=O)O)C(=O)OC(C)(C)C)n1]

Batch 1: To methyl 2-((2-(((tert-butoxycarbonyl)(2-(6-methoxy-3-nitropyridin-2-yl)ethyl)amino)methyl)-4-fluorophenyl)amino)-5-(trifluoromethyl)benzoate (1.87 g, 3.00 mmol) was added THF (20 mL) and H₂O (6.67 mL), followed by LiOH (0.432 g, 18.0 mmol), and the reaction mixture was stirred at 50° C. for 22 h. The reaction mixture was cooled to ambient temperature. Batch 2: To methyl 2-((2-(((tert-butoxycarbonyl)(2-(6-methoxy-3-nitropyridin-2-yl)ethyl)amino)methyl)-4-fluorophenyl)amino)-5-(trifluoromethyl)benzoate (6.52 g, 10.1 mmol) was added THF (80 mL) and H₂O (27 mL), followed by LiOH (1.50 g, 62.6 mmol), and the reaction mixture was stirred at 50° C. for 22 h. The reaction mixture was cooled to ambient temperature, combined with batch 1, and the reaction mixture acidified with 1N HCl and extracted with EtOAc. The layers were separated, the organic phase was washed with H₂O, brine, dried over Na₂SO₄, filtered and the solvent evaporated under reduced pressure to afford 2-((2-(((tert-Butoxycarbonyl)(2-(6-methoxy-3-nitropyridin-2-yl)ethyl)amino)methyl)-4-fluorophenyl)amino-5-(trifluoromethyl)benzoic acid 41c (6.42 g, 98% yield) as a beige foam. HPLC/MS 1.49 min (A), [M+H]⁺ 609.2. ¹H NMR (DMSO-d₆, 400 MHz) δ 13.0-13.8 (m, 1H), 9.63 (s, 1H), 8.2-8.4 (m, 1H), 8.11 (s, 1H), 7.5-7.7 (m, 1H), 7.3-7.4 (m, 1H), 7.21 (br d, 1H, J=2.9 Hz), 7.0-7.1 (m, 1H), 6.83 (br s, 1H), 6.57 (br d, 1H, J=8.3 Hz), 4.2-4.4 (m, 2H), 3.82 (s, 3H), 3.5-3.7 (m, 2H), 3.0-3.3 (m, 2H), 1.1-1.2 (m, 11H).

### Step D: 2-((2-(((2-(3-Amino-6-methoxypyridin-2-yl)ethyl)(tert-butoxycarbonyl)amino)-methyl)-4-fluorophenyl)amino-5-(trifluoromethyl)benzoic acid 41d

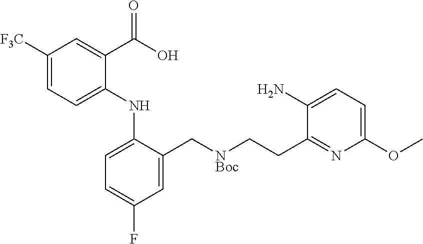 [SMILES: COc1ccc(N)c(CCN(Cc2cc(F)ccc2Nc2ccc(C(F)(F)F)cc2C(=O)O)C(=O)OC(C)(C)C)n1]

To a solution of 2-((2-(((tert-butoxycarbonyl)(2-(6-methoxy-3-nitropyridin-2-yl)ethyl)amino)methyl)-4-fluorophenyl)amino)-5-(trifluoromethyl)benzoic acid (6.25 g, 10.3 mmol), in MeOH (150 mL), was added NH₄Cl (3.30 g, 61.6 mmol), H₂O (50.0 mL) and zinc (10.1 g, 154 mmol) at room temperature and the reaction mixture was stirred at 60° C. for 1 h. The reaction mixture was cooled to ambient temperature, filtered through a pad of celite, washed with MeOH and the solvent concentrated under reduced pressure. The solid was partitioned between EtOAc and H₂O, the layers separated, the aqueous phase extracted with EtOAc, the combined organic extracts washed with H₂O, brine, dried over Na₂SO₄, filtered and the solvent evaporated under reduced pressure to afford 2-((2-(((2-(3-amino-6-methoxypyridin-2-yl)ethyl)(tert-butyoxycarbonyl)amino)methyl)-4-fluorophenyl)amino-5-(trifluoromethyl)benzoic acid 41d (5.85 g, 88% yield) as a beige solid. HPLC/MS 1.07 min (A), [M+H]⁺ 579.2. ¹H NMR (DMSO-d₆, 400 MHz) δ 9.61 (s, 1H), 8.11 (br s, 2H), 7.56 (br d, 2H, J=8.8 Hz), 7.35 (dd, 1H, J=5.4, 8.8 Hz), 7.21 (dt, 1H, J=3.2, 8.4 Hz), 7.0-7.1 (m, 1H), 6.95 (d, 1H, J=8.8 Hz), 6.4-6.6 (m, 1H), 6.35 (d, 1H, J=8.3 Hz), 4.31 (br s, 2H), 3.60 (s, 3H), 3.3-3.6 (m, 3H), 2.71 (br t, 2H, J=6.8 Hz), 1.27 (br s, 11H).

### Step E: tert-Butyl 8-fluoro-15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 41e

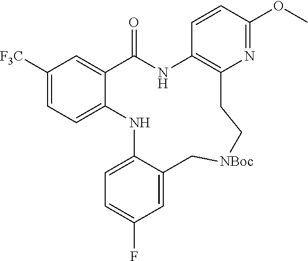 [SMILES: COc1ccc2c(n1)CCN(C(=O)OC(C)(C)C)Cc1cc(F)ccc1Nc1ccc(C(F)(F)F)cc1C(=O)N2]

To a solution of 2-((2-(((2-(3-amino-6-methoxypyridin-2-yl)ethyl)(tert-butoxycarbonyl)amino)methyl)-4-fluorophenyl)amino)-5-(trifluoromethyl)benzoic acid (5.85 g, 10.1 mmol), DMF (50 ml) was added HATU (5.77 g, 15.2 mmol), followed by DIEA (5.30 ml, 30.3 mmol), and the reaction mixture stirred at room temperature for 3 h. The reaction mixture was diluted with EtOAc, washed with H₂O (2×), brine, dried over Na₂SO₄, filtered and the solvent evaporated under reduced pressure. The crude residue was dissolved in DCM, absorbed onto a silica gel packed precolumn and purified by silica gel flash column chromatography (330 g) eluting with a 100% heptane to 50% EtOAc-hetpanes gradient. Product fractions were combined and evaporated under reduced pressure to afford tert-butyl 8-fluoro-15-methoxy-19-oxo-2-(trifluoromethyl)-5,10,12,13,18,19-hexahydro-11H-dibenzo[c,j]pyrido[3,2-j][1,5,9]triazacyclotridecine-11-carboxylate 41e (2.51 g, 31% yield) as a white solid, in 70% purity. HPLC/MS 1.38 min (A), [M+H]⁺ 561.2.

### Step F: tert-Butyl 8-fluoro-15-methoxy-19-oxo-2-(trifluoromethyl)-12,13-dihydro-19H-5,18-methano dibenzo[c,f]pyrido[3,2-j][1,5,9]triazacyclotridecine-11(10H)-carboxylate 41f

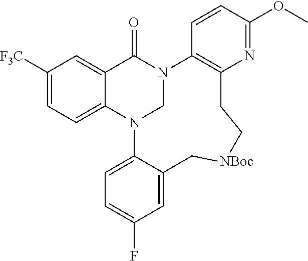 [SMILES: COc1ccc2c(n1)CCN(C(=O)OC(C)(C)C)Cc1cc(F)ccc1N1CN2C(=O)c2cc(C(F)(F)F)ccc21]

To a solution of tert-butyl 8-fluoro-15-methoxy-19-oxo-2-(trifluoromethyl)-5,10,12,13,18,19-hexahydro-11H-dibenzo[c,j]pyrido[3,2-j][1,5,9]triazacyclotridecine-11-carboxylate (2.51 g, 4.48 mmol) in MeCN (50 mL) was added Cs₂CO₃ (8.75 g, 26.9 mmol), followed by CH₂I₂ (5.41 mL, 67.2 mmol) and the reaction mixture was stirred at 90° C. for 19 h. The reaction mixture was cooled to ambient temperature, diluted with EtOAc, washed with H₂O, brine, dried over Na₂SO₄, filtered and the solvent evaporated under reduced pressure. The crude residue was dissolved in DCM, absorbed onto a silica gel packed precolumn and purified by silica gel flash column chromatography (220 g) eluting with a 100% heptane to 100% EtOAc gradient.

Product fractions were combined and evaporated under reduced pressure. The material was further purified by silica gel flash column chromatography (220 g) eluting with a 100% DCM to 10% 3:1 EtOAc/EtOH-DCM gradient. Product fractions were combined and evaporated under reduced pressure to afford tert-butyl 8-fluoro-15-methoxy-19-oxo-2-(trifluoromethyl)-12,13-dihydro-19H-5,18-methano dibenzo[c,j]pyrido[3,2-j][1,5,9]triazacyclotridecine-11(10H)-carboxylate 41f (1.18 g, 43% yield) as a white solid. HPLC/MS 1.40 min (A), [M+H]⁺ 573.5. ¹H NMR (DMSO-d₆, 400 MHz) δ 8.13 (d, 1H, J=2.0 Hz), 7.73 (d, 1H, J=8.3 Hz), 7.7-7.7 (m, 1H), 7.5-7.6 (m, 1H), 7.4-7.4 (m, 1H), 7.3-7.4 (m, 1H), 6.80 (d, 1H, J=8.3 Hz), 6.3-6.5 (m, 1H), 5.6-5.7 (m, 1H), 4.8-5.0 (m, 1H), 4.6-4.8 (m, 1H), 3.9-4.0 (m, 1H), 3.86 (s, 3H), 3.6-3.8 (m, 1H), 2.9-3.0 (m, 1H), 2.6-2.8 (m, 1H), 2.5-2.6 (m, 1H), 1.45 (s, 9H).

### Step G: 8-Fluoro-2-(trifluoromethyl)-10,11,12,13-tetrahydro-19H-5,18-methanodibenzo[c,f]pyrido[3,2-j][1,5,9]triazacyclotridecine-15,19(14H)-dione

### Example 41

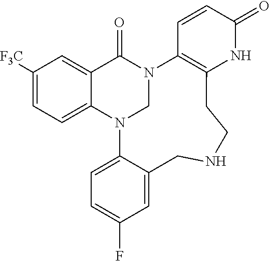 [SMILES: O=C1c2cc(C(F)(F)F)ccc2N2CN1c1ccc(=O)[nH]c1CCNCc1cc(F)ccc12]

To a solution of tert-butyl 8-fluoro-15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate (1.18 g, 2.06 mmol), in isopropanol (20 mL) was added HCl (5N in IPA) (20.6 mL, 103 mmol) and the reaction mixture was stirred at 90° C. for 24 h. The reaction mixture was cooled to ambient temperature, the solvent evaporated under reduced pressure, and the residual solid partitioned between DCM and 10% NaHCO₃. The layers were separated, the organic phase dried over Na₂SO₄, filtered and the solvent evaporated under reduced pressure. The solid was triturated with diethyl ether, the solid filtered, washed with diethyl ether and dried under vacuo to afford 8-fluoro-2-(trifluoromethyl)-10,11,12,13-tetrahydro-19H-5,18-methano dibenzo[c,f]pyrido[3,2-j][1,5,9]triazacyclotridecine-15,19(14H)-dione (0.734 g, 75% yield) as a white solid. HPLC/MS 0.64 min (A), [M+H]⁺ 459.1. ¹H NMR (DMSO-d₆, 400 MHz) δ 11.5-12.1 (m, 1H), 8.09 (d, 1H, J=1.5 Hz), 7.63 (dd, 1H, J=2.4, 8.8 Hz), 7.56 (dd, 1H, J=5.4, 8.3 Hz), 7.4-7.5 (m, 2H), 7.31 (dt, 1H, J=2.7, 8.4 Hz), 6.55 (d, 1H, J=8.8 Hz), 6.22 (br d, 1H, J=9.3 Hz), 5.40 (d, 1H, J=9.3 Hz), 5.12 (d, 1H, J=9.8 Hz), 3.85 (br s, 1H), 3.4-3.4 (m, 1H), 2.7-3.1 (m, 3H), 2.40 (br s, 1H), 2.1-2.3 (m, 1H).

### Example 42

### 2-Chloro-8-fluoro-10,11,12,13-tetrahydro-19H-5,18-methanodibenzo[c]dipyrido[2,3-f:3′,2′-j][1,5,9]triazacyclotridecine-15,19(14H)-dione

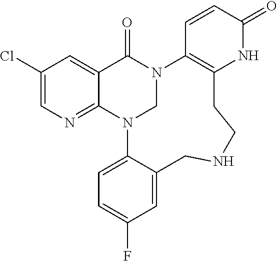 [SMILES: O=C1c2cc(Cl)cnc2N2CN1c1ccc(=O)[nH]c1CCNCc1cc(F)ccc12]

### Step A: Methyl 5-chloro-2-((4-fluoro-2-formylphenyl)amino)nicotinate 42a

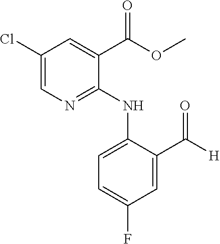 [SMILES: COC(=O)c1cc(Cl)cnc1Nc1ccc(F)cc1C=O]

Following the procedure outlined in Example 41, Step A, substituting methyl 2-bromo-5-(trifluoromethyl)benzoate with methyl 2-bromo-5-chloronicotinate, BINAP with Xantphos, and Pd(OAc)₂ with Pd₂(dba)₃, and stirring the reaction mixture at 100° C. for 4 h, methyl 5-chloro-2-((4-fluoro-2-formylphenyl)amino)nicotinate 42a (3.50 g, 62% yield) was prepared. HPLC/MS 1.25 min (B), [M+H]⁺ 309.0. ¹H NMR (DMSO-d₆, 400 MHz) δ 11.61 (s, 1H), 9.96 (s, 1H), 8.49 (d, 2H, J=2.9 Hz), 8.29 (d, 1H, J=2.9 Hz), 7.7-7.9 (m, 1H), 7.4-7.6 (m, 1H), 3.96 (s, 3H).

### Step B: Methyl 2-((2-(((tert-butoxycarbonyl)(2-(6-methoxy-3-nitropyridin-2-yl)ethyl)-amino)methyl)-4-fluorophenyl)amino)-5-chloronicotinate 42b

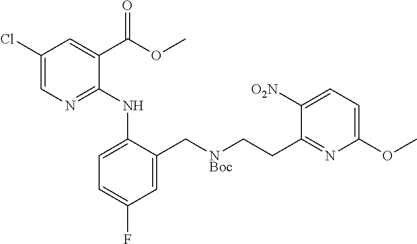 [SMILES: COC(=O)c1cc(Cl)cnc1Nc1ccc(F)cc1CN(CCc1nc(OC)ccc1[N+](=O)[O-])C(=O)OC(C)(C)C]

Following the procedure outlined in Example 42, Step B, methyl 2-((2-(((tert-butoxycarbonyl)(2-(6-methoxy-3-nitropyridin-2-yl)ethyl)amino)methyl)-4-fluorophenyl)amino)-5-chloronicotinate 42b (3.45 g, 45% yield) was prepared as a yellow solid. HPLC/MS 1.55 min (B), [M+H]⁺ 590.1. ¹H NMR (DMSO-d₆, 400 MHz) δ 9.43 (s, 1H), 8.2-8.4 (m, 2H), 8.17 (d, 1H, J=2.9 Hz), 7.4-7.6 (m, 1H), 7.13 (dt, 1H, J=2.9, 8.6 Hz), 6.9-7.0 (m, 1H), 6.84 (br d, 1H, J=7.3 Hz), 4.2-4.4 (m, 2H), 3.91 (s, 3H), 3.87 (s, 3H), 3.5-3.7 (m, 2H), 3.15 (br s, 2H), 1.23 (s, 9H).

### Step C: 2-((2-(((tert-Butoxycarbonyl)(2-(6-methoxy-3-nitropyridin-2-yl)ethyl)amino)-methyl)-4-fluorophenyl)amino)-5-chloronicotinic acid 42c

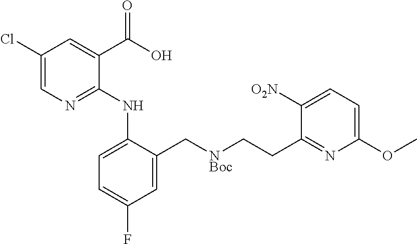 [SMILES: COc1ccc([N+](=O)[O-])c(CCN(Cc2cc(F)ccc2Nc2ncc(Cl)cc2C(=O)O)C(=O)OC(C)(C)C)n1]

Following the procedure outlined in Example 42, Step C, stirring the reaction mixture at 50° C. for 3 h, 2-((2-(((tert-butoxycarbonyl)(2-(6-methoxy-3-nitropyridin-2-yl)ethyl)amino)methyl)-4-fluorophenyl) amino)-5-chloronicotinic acid 42c (478 mg, 99% yield) was prepared as a solid. HPLC/MS 0.97 min (B), [M+H]⁺ 576.1. ¹H NMR (DMSO-d₆, 400 MHz) δ 13.65-13.97 (m, 1H) 9.65-9.82 (m, 1H) 8.28-8.35 (m, 1H) 8.20-8.26 (m, 1H) 8.15 (d, J=2.45 Hz, 1H) 7.55 (br s, 1H) 7.12 (td, J=8.56, 2.93 Hz, 1H) 6.96 (dd, J=9.78, 2.93 Hz, 1H) 6.85 (br d, J=7.82 Hz, 1H) 4.21-4.36 (m, 2H) 3.86 (s, 3H) 3.56-3.67 (m, 2H) 3.06-3.21 (m, 2H) 1.19-1.30 (m, 9H).

### Step D: 2-((2-(((2-(3-Amino-6-methoxypyridin-2-yl)ethyl)(tert-butoxycarbonyl)amino)-methyl)-4-fluoro phenyl)amino)-5-chloronicotinic acid 42d

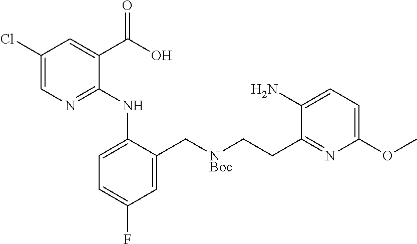 [SMILES: COc1ccc(N)c(CCN(Cc2cc(F)ccc2Nc2ncc(Cl)cc2C(=O)O)C(=O)OC(C)(C)C)n1]

Following the procedure outlined in Example 42, Step D, 2-((2-(((2-(3-amino-6-methoxypyridin-2-yl)ethyl)(tert-butoxycarbonyl)amino)methyl)-4-fluorophenyl)amino)-5-chloronicotinic acid 42d (2.36 g, 90% yield) was prepared as a beige solid. HPLC/MS 0.86 min (B), [M+H]⁺ 546.1. ¹H NMR (DMSO-d₆, 400 MHz) δ 10.8-11.3 (m, 1H), 8.0-8.3 (m, 2H), 7.6-7.9 (m, 1H), 7.0-7.1 (m, 1H), 6.9-7.0 (m, 1H), 6.8-6.9 (m, 1H), 6.34 (d, 1H, J=8.3 Hz), 4.5-4.7 (m, 2H), 4.3-4.5 (m, 2H), 3.59 (s, 3H), 3.4-3.6 (m, 3H), 2.6-2.8 (m, 2H), 1.35 (s, 9H).

### Step E: tert-Butyl 2-chloro-8-fluoro-15-methoxy-19-oxo-5,10,12,13,18,19-hexahydro-11H-benzo[c]dipyrido[2,3-f3′,2′-j][1,5,9]triazacyclotridecine-11-carboxylate 42e

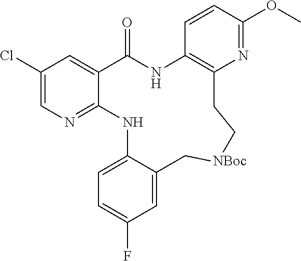 [SMILES: COc1ccc2c(n1)CCN(C(=O)OC(C)(C)C)Cc1cc(F)ccc1Nc1ncc(Cl)cc1C(=O)N2]

Following the procedure outlined in Example 42, Step E, substituting HATU with pyoxim and stirring the reaction mixture at room temperature for 18 h, tert-butyl 2-chloro-8-fluoro-15-methoxy-19-oxo-2-(trifluoromethyl)-5,10,12,13,18,19-hexahydro-11H-benzo[c]dipyrido[2,3-j:3′,2′-j][1,5,9]triazacyclotri decine-11-carboxylate 42e (250 mg, 17% yield) was prepared as a yellow solid. HPLC/MS 1.29 min (B), [M+H]⁺ 528.1. ¹H NMR (DMSO-d₆, 400 MHz) δ 10.05 (s, 1H), 8.2-8.3 (m, 1H), 8.0-8.1 (m, 2H), 7.94 (d, 1H, J=2.4 Hz), 7.2-7.4 (m, 2H), 7.0-7.2 (m, 1H), 6.75 (d, 1H, J=8.3 Hz), 4.3-4.6 (m, 2H), 3.83 (s, 3H), 3.4-3.5 (m, 2H), 2.9-3.0 (m, 2H), 1.48 (s, 9H).

### Step F: tert-Butyl 2-chloro-8-fluoro-15-methoxy-19-oxo-12,13-dihydro-19H-5,18-methanobenzo[c]dipyrido[2,3-f:3′,2′-j][1,5,9]triazacyclotridecine-11(10H)-carboxylate 42f

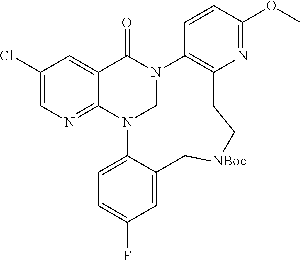 [SMILES: COc1ccc2c(n1)CCN(C(=O)OC(C)(C)C)Cc1cc(F)ccc1N1CN2C(=O)c2cc(Cl)cnc21]

Following the procedure outlined in Example 42, Step F, stirring the reaction mixture at 90° C. for 24 h, tert-butyl 2-chloro-8-fluoro-15-methoxy-19-oxo-12,13-dihydro-19H-5,18-methanobenzo[c]dipyrido[2,3-j:3′,2′-j][1,5,9]triazacyclo tridecine-11(10H)-carboxylate 42f (193 mg, 47% yield) was prepared as a white solid. HPLC/MS 1.30 min (B), [M+H]⁺ 540.1. ¹H NMR (DMSO-d₆, 400 MHz) δ 8.32 (d, 1H, J=2.4 Hz), 8.2-8.2 (m, 1H), 7.72 (d, 1H, J=8.8 Hz), 7.42 (dd, 1H, J=5.6, 8.6 Hz), 7.3-7.3 (m, 1H), 7.2-7.3 (m, 1H), 5.71 (d, 1H, J=11.7 Hz), 4.6-5.0 (m, 2H), 3.85 (s, 3H), 3.5-3.8 (m, 2H), 2.8-3.1 (m, 2H), 1.44 (s, 9H), 1.25 (s, 2H).

### Step G: 2-Chloro-8-fluoro-10,11,12,13-tetrahydro-19H-5,18-methanobenzo[c]-dipyrido[2,3-f:3′,2′-][1,5,9]triazacyclotridecine-15,19(14H)-dione Example 42

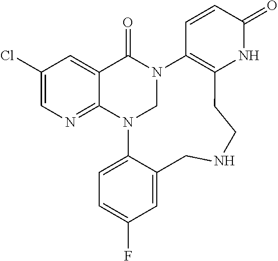 [SMILES: O=C1c2cc(Cl)cnc2N2CN1c1ccc(=O)[nH]c1CCNCc1cc(F)ccc12]

Following the procedure outlined in Example 42, Step G, stirring the reaction mixture at 90° C. for 18 h, 2-chloro-8-fluoro-10,11,12,13-tetrahydro-19H-5,18-methanobenzo[c]-dipyrido[2,3-j:3′,2′-j][1,5,9]triazacyclotridecine-15,19(14H)-dione (193 mg, 47% yield) was prepared as a white solid. HPLC/MS 0.83 min (B), [M+H]⁺ 426.2. ¹H NMR (DMSO-d₆, 400 MHz) δ 11.7-12.0 (m, 1H), 8.2-8.4 (m, 1H), 8.1-8.2 (m, 1H), 7.4-7.6 (m, 2H), 7.3-7.4 (m, 2H), 7.2-7.4 (m, 1H), 6.1-6.4 (m, 1H), 5.4-5.6 (m, 1H), 5.1-5.4 (m, 1H), 3.9-4.1 (m, 2H), 2.9-3.1 (m, 4H).

### Example 43

### 2-Chloro-3,8-difluoro-10,11,12,13-tetrahydro-19H-5,18-methanodibenzo[c,f]pyrido[3,2-f][1,5,9]triazacyclotridecine-15,19(14H)-dione

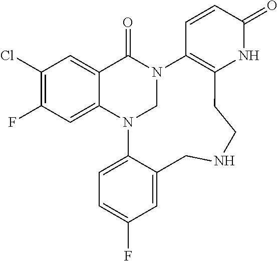 [SMILES: O=C1c2cc(Cl)c(F)cc2N2CN1c1ccc(=O)[nH]c1CCNCc1cc(F)ccc12]

### Step A: Methyl 5-chloro-4-fluoro-2-((4-fluoro-2-formylphenyl)amino)benzoate 43a

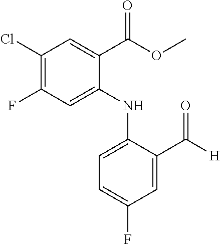 [SMILES: COC(=O)c1cc(Cl)c(F)cc1Nc1ccc(F)cc1C=O]

Following the procedure outlined in Example 42, Step A, substituting methyl 2-bromo-5-(trifluoromethyl)benzoate with methyl 2-bromo-5-chloro-4-fluorobenzoate, and Pd(OAc)₂ with Pd₂(dba)₃, and stirring the reaction mixture at 80° C. for 21 h, to which additional Pd₂(dba)₃ and BINAP were added and the reaction mixture continued at 80° C. for 5 h, methyl 5-chloro-4-fluoro-2-((4-fluoro-2-formyl phenyl)amino)benzoate 43a (6.18 g, 42% yield) was prepared. HPLC/MS 1.28 min (A), [M+H]⁺ 326.1. ¹H NMR (DMSO-d₆, 400 MHz) δ 10.80 (s, 1H), 9.97 (s, 1H), 8.05 (d, 1H, J=8.3 Hz), 7.78 (dd, 1H, J=3.2, 8.6 Hz), 7.63 (dd, 1H, J=4.4, 9.3 Hz), 7.51 (dt, 1H, J=2.9, 8.6 Hz), 7.37 (d, 1H, J=11.7 Hz), 3.89 (s, 3H).

### Step B: Methyl 2-((2-(((tert-butoxycarbonyl)(2-(6-methoxy-3-nitropyridin-2-yl)ethyl)-amino)methyl)-4-fluorophenyl)amino)-5-chloro-4-fluorobenzoate 43b

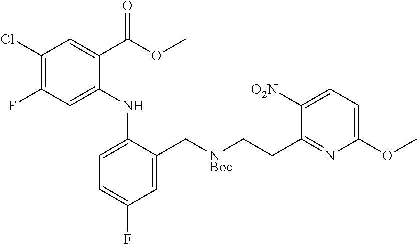 [SMILES: COC(=O)c1cc(Cl)c(F)cc1Nc1ccc(F)cc1CN(CCc1nc(OC)ccc1[N+](=O)[O-])C(=O)OC(C)(C)C]

Batch 1: 2-(6-Methoxy-3-nitropyridin-2-yl)ethan-1-amine, dihydrochloride (581 mg, 2.15 mmol) was dissolved in DCE (20 mL) and 1N NaOH (20 mL), separated the organics, the aqueous extracted with DCE (10 mL), and the combined organics dried over MgSO₄, filtered, added to methyl 5-chloro-4-fluoro-2-((4-fluoro-2-formylphenyl)amino)benzoate (667 mg, 2.05 mmol), and the reaction mixture was stirred at RT for 30 min. Sodium triacetoxyborohydride (1.30 g, 6.14 mmol) was added and the reaction mixture stirred for 4 h. Boc₂O (1.34 g, 6.14 mmol) was added and the reaction mixture stirred for 2 h. Batch 2: 2-(6-Methoxy-3-nitropyridin-2-yl)ethan-1-amine, dihydrochloride (4.79 g, 17.7 mmol) was dissolved in DCE (100 ml) and 1N NaOH (100 mL), separated the organics, the aqueous phase extracted with DCE (70 mL), and the combined organic extracts dried over MgSO₄, filtered, added to methyl 5-chloro-4-fluoro-2-((4-fluoro-2-formylphenyl)amino)benzoate (5.5 g, 16.9 mmol), and the reaction mixture was stirred at room temperature for 30 min. Sodium triacetoxyborohydride (10.7 g, 50.7 mmol) was added and the reaction mixture stirred for 18 h. Boc₂O (11.1 g, 50.7 mmol) was added and the reaction mixture stirred for 2 h. Batch 1 and batch 2 were combined, preabsorbed onto silica gel, and purified by flash column chromatography (80 g), eluting with a 100% heptanes to 40% in EtOAc-heptanes gradient. Product fractions were combined and evaporated under reduced pressure to afford 2-(((tert-butoxycarbonyl)(2-(6-methoxy-3-nitropyridin-2-yl)ethyl)amino)methyl)-4-fluorophenyl)amino)-5-chloro-4-fluorobenzoate 43b (8.8 g, 73% yield). HPLC/MS 1.66 min (B), [M+H]⁺ 607.2. ¹H NMR (DMSO-d₆, 400 MHz) δ 9.12 (br s, 1H), 8.30 (br d, 1H, J=8.8 Hz), 7.96 (d, 1H, J=8.3 Hz), 7.34 (br dd, 1H, J=5.6, 8.6 Hz), 7.21 (dt, 1H, J=2.9, 8.3 Hz), 7.09 (br s, 1H), 6.8-6.9 (m, 1H), 5.9-6.5 (m, 1H), 4.2-4.4 (m, 2H), 3.9-3.9 (m, 3H), 3.8-3.9 (m, 3H), 3.5-3.7 (m, 2H), 3.1-3.2 (m, 2H), 1.20 (s, 9H).

### Step C: 2-((2-(((tert-Butoxycarbonyl)(2-(6-methoxy-3-nitropyridin-2-yl)ethyl)amino)-methyl)-4-fluorophenyl)amino)-5-chloro-4-fluorobenzoic acid 43c

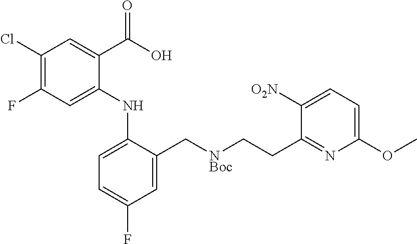 [SMILES: COc1ccc([N+](=O)[O-])c(CCN(Cc2cc(F)ccc2Nc2cc(F)c(Cl)cc2C(=O)O)C(=O)OC(C)(C)C)n1]

Following the procedure outlined in Example 42, Step C, stirring the reaction mixture at 50° C. for 5 h, 2-((2-(((tert-butoxycarbonyl)(2-(6-methoxy-3-nitropyridin-2-yl)ethyl)amino)methyl)-4-fluorophenyl) amino)-5-chloro-4-fluorobenzoic acid 43c (8.60 g, 100% yield) was prepared. HPLC/MS 1.06 min (B), [M+H]⁺ 593.1. ¹H NMR (DMSO-d₆, 400 MHz) S 9.9-10.0 (m, 1H), 9.43 (br s, 1H), 8.29 (d, 1H, J=9.3 Hz), 7.93 (d, 1H, J=8.3 Hz), 7.35 (br dd, 1H, J=5.4, 8.8 Hz), 7.19 (dt, 1H, J=2.9, 8.6 Hz), 7.06 (br d, 1H, J=2.9 Hz), 6.83 (br s, 1H), 6.1-6.4 (m, 1H), 4.2-4.4 (m, 2H), 3.85 (s, 3H), 3.6-3.7 (m, 2H), 3.3-3.5 (m, 2H), 1.2-1.3 (m, 9H).

### Step D: 2-((2-(((2-(3-Amino-6-methoxypyridin-2-yl)ethyl)(tert-butyloxycarbonyl)amino)-methyl)-4-fluoro phenyl)amino-5-chloro-4-fluorobenzoic acid 43d

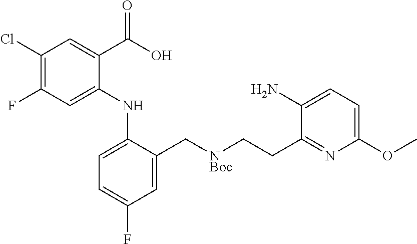 [SMILES: COc1ccc(N)c(CCN(Cc2cc(F)ccc2Nc2cc(F)c(Cl)cc2C(=O)O)C(=O)OC(C)(C)C)n1]

Following the procedure outlined in Example 42, Step D, stirring the reaction mixture at 60° C. for 5 h, 2-((2-(((2-(3-amino-6-methoxypyridin-2-yl)ethyl)(tert-butyloxycarbonyl)amino)methyl)-4-fluorophenyl) amino-5-chloro-4-fluorobenzoic acid 43d (7.70 g, 94% yield) was prepared. HPLC/MS 0.96 min (B), [M+H]⁺ 563.2.

### Step E: tert-Butyl 2-chloro-3,8-difluoro-15-methoxy-19-oxo-5,10,12,13,18,19-hexahydro-11H-dibenzo [c,f]pyrido[3,2-j][1,5,9]triazacyclotridecine-11-carboxylate 43e

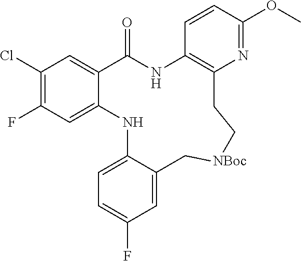 [SMILES: COc1ccc2c(n1)CCN(C(=O)OC(C)(C)C)Cc1cc(F)ccc1Nc1cc(F)c(Cl)cc1C(=O)N2]

Following the procedure outlined in Example 42, Step E, stirring the reaction mixture at room temperature for 30 min, tert-butyl 2-chloro-3,8-difluoro-15-methoxy-19-oxo-5,10,12,13,18,19-hexahydro-11H-dibenzo[c,f]pyrido[3,2-j][1,5,9]triazacyclotridecine-11-carboxylate 43e (426 mg, 40% yield) was prepared as a beige solid, in 65% purity. HPLC/MS 1.44 min (B), [M+H]⁺ 545.1.

### Step F: tert-Butyl 2-chloro-3,8-difluoro-15-methoxy-19-oxo-12,13-dihydro-19H-5,18-methanodibenzo [c,f]pyrido[3,2-j][1,5,9]triazacyclotridecine-11(10H)-carboxylate 43

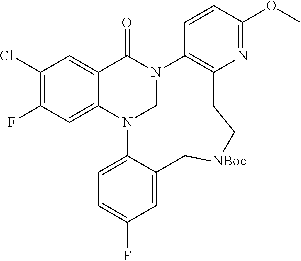 [SMILES: COc1ccc2c(n1)CCN(C(=O)OC(C)(C)C)Cc1cc(F)ccc1N1CN2C(=O)c2cc(Cl)c(F)cc21]

Following the procedure outlined in Example 42, Step F, stirring the reaction mixture at 90° C. for 24 h, tert-butyl 2-chloro-3,8-difluoro-15-methoxy-19-oxo-12,13-dihydro-19H-5,18-methanodibenzo[c,f]pyrido [3,2-j][1,5,9]triazacyclotridecine-11(10H)-carboxylate 43f (115 mg, 46% yield) was prepared. HPLC/MS 1.46 min (B), [M+H]⁺ 557.2.

### Step G: 2-Chloro-3,8-difluoro-10,11,12,13-tetrahydro-19H-5,18-methanodibenzo[c,f]pyrido[3,2-j][1,5,9]triazacyclotridecine-15,19(14H)-dione Example 43

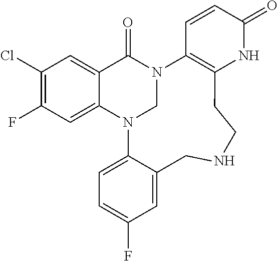 [SMILES: O=C1c2cc(Cl)c(F)cc2N2CN1c1ccc(=O)[nH]c1CCNCc1cc(F)ccc12]

Following the procedure outlined in Example 42, Step G, stirring the reaction mixture at 90° C. for 18 h, 2-chloro-3,8-difluoro-10,11,12,13-tetrahydro-19H-5,18-methanodibenzo[c,f]pyrido[3,2-j][1,5,9]triazacyclo tridecine-15,19(14H)-dione (73 mg, 80% yield) was prepared as a white solid. HPLC/MS 1.00 min (B), [M+H]⁺ 443.2. ¹H NMR (DMSO-d₆, 400 MHz) δ 10.73 (br d, 1H, J=2.0 Hz), 8.9-9.1 (m, 1H), 7.98 (d, 1H, J=7.8 Hz), 7.77 (dd, 1H, J=3.4, 9.3 Hz), 7.59 (dd, 1H, J=5.4, 8.8 Hz), 7.5-7.5 (m, 1H), 7.44 (d, 1H, J=9.3 Hz), 6.34 (ddd, 2H, J=9.8, 39.1, 41.1 Hz), 4.8-5.6 (m, 2H), 3.9-4.2 (m, 2H), 3.0-3.3 (m, 2H), 2.6-3.0 (m, 2H).

### Example 44

### 2,8-Difluoro-3-(trifluoromethyl)-10,11,12,13-tetrahydro-19H-5,18-methanodibenzo[c,f]pyrido[3,2-i][1,5,9]triazacyclotridecine-15,19(14H)-dione

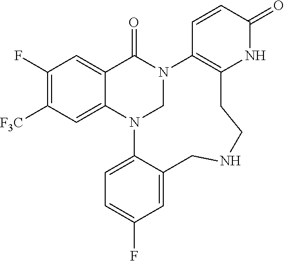 [SMILES: O=C1c2cc(F)c(C(F)(F)F)cc2N2CN1c1ccc(=O)[nH]c1CCNCc1cc(F)ccc12]

### Step A: Methyl 5-fluoro-2-((4-fluoro-2-formylphenyl)amino)-4-(trifluoromethyl)-benzoate 44a

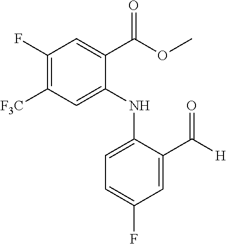 [SMILES: COC(=O)c1cc(F)c(C(F)(F)F)cc1Nc1ccc(F)cc1C=O]

To a 100 ml round bottomed flask was added a solution of methyl 2-amino-5-fluoro-4-(trifluoromethyl)benzoate Int-2j (1.96 g, 8.28 mmol) and 2-bromo-5-fluorobenzaldehyde (1.20 g, 5.91 mmol) in 1,4-dioxane (40 mL), followed by Cs₂CO₃ (2.70 g, 8.28 mmol), Pd(OAc)₂ (0.066 g, 0.296 mmol)) and Xantphos (0.342 g, 0.591 mmol). The reaction mixture was purged with N₂ for 5 min and then heated at 80° C. for 2 h. The reaction mixture was cooled to ambient temperature, diluted with EtOAc and sat'd aq. NH₄Cl, and the layers separated, and the aqueous phase further extracted with EtOAc (3×10 mL). The organic extracts were combined, washed with brine, dried over MgSO₄, filtered and concentrated under reduced pressure. The residue was dissolved in DCM, adsorbed on a silica gel pre-column and purified by flash column chromatography (220 g), eluting with a 100% heptanes to 100% EtOAc gradient. The product fractions were collected and evaporated under reduced pressure to afford methyl 5-fluoro-2-((4-fluoro-2-formylphenyl)amino)-4-(trifluoromethyl)benzoate 44a (2.00 g, 89% yield) as a yellow solid. HPLC/MS 1.31 min (B), [M+H]⁺ 359.1. ¹H NMR (CDCl₃, 400 MHz) δ 10.98 (s, 1H), 9.98 (s, 1H), 7.83 (d, J=10.8 Hz, 1H), 7.64-7.76 (m, 1H), 7.37-7.47 (m, 2H), 7.21-7.34 (m, 1H), 4.00 (s, 3H).

### Step B: Methyl 2-((2-(((tert-butoxycarbonyl(2-(6-methoxy-3-nitropyridin-2-yl)ethyl)amino)methyl)-4-fluorophenyl)amino)-5-fluoro-4-(trifluoromethyl)benzoate 44b

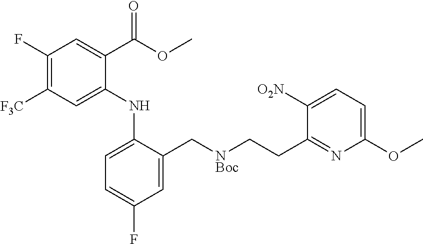 [SMILES: COC(=O)c1cc(F)c(C(F)(F)F)cc1Nc1ccc(F)cc1CN(CCc1nc(OC)ccc1[N+](=O)[O-])C(=O)OC(C)(C)C]

To a solution of 2-(6-methoxy-3-nitropyridin-2-yl)ethan-1-amine 2,2,2-trifluoroacetate (9876 mg, 3.17 mmol) in THF (30 ml), at room temperature, was added TEA (1.70 mL, 12.2 mmol), and MgSO₄ (293.8 mg, 2.44 mmol), and the reaction mixture was stirred for 30 min. Methyl 5-fluoro-2-((4-fluoro-2-formylphenyl)amino)-4-(trifluoromethyl)benzoate (877.0 mg, 2.44 mmol) was added and the reaction mixture stirred for 1 h. The solution was diluted with EtOAc and H₂O, the layers separated, and the aqueous phase extracted with EtOAc. The combined organic extracts were washed with brine, dried over MgSO₄, filtered and the solvent evaporated under reduced pressure to afford methyl (E)-5-fluoro-2((4-fluror-2-(((2-(6-methoxy-3-nitropyridin-2-yl)ethyl)imino)methyl)phenyl)amino)-4-(trifluoromethyl) benzoate 44b (1.49 g, >100% yield) as a yellow solid. HPLC/MS 1.60 min (B), [M+H]⁺ 539.1. 1H NMR (400 MHz, CDCl₃) δ 10.92 (s, 1H) 8.32 (s, 1H) 8.05 (d, J=9.29 Hz, 1H) 7.64 (d, J=10.76 Hz, 1H) 7.43 (d, J=5.87 Hz, 1H) 7.19 (br d, J=4.40 Hz, 1H) 7.11 (dd, J=8.80, 2.93 Hz, 1H) 6.87-7.00 (m, 1H) 6.48 (d, J=8.80 Hz, 1H) 4.04-4.12 (m, 2H) 3.83 (s, 3H) 3.80-3.81 (m, 3H) 3.54 (t, J=6.85 Hz, 2H). To a solution of methyl (E)-5-fluoro-2-((4-fluoro-2-(((2-(6-methoxy-3-nitropyridin-2-yl)ethyl)imino)methyl)phenyl)amino)-4-(trifluoromethyl) benzoate (1.31 g, 2.43 mmol) in DCM (25 mL), at room temperature, was added NaBH(OAC)₃ (1.55 g, 7.29 mmol) and the reaction mixture was stirred for 20.5 h. 1M sodium hydroxide (24.3 mL, 24.3 mmol) was added to the reaction mixture and and stirred vigorously for 2 h. Di-tert-butyl dicarbonate (5.31 g, 24.3 mmol) was added to the reaction mixture and the solution was stirred vigorously for 1 h. The reaction mixture was diluted with EtOAC and H₂O, the layers separated, and the aqueous phase extracted with EtOAc. The combined organic extracts were washed with brine, dried over MgSO₄, filtered and the solvent evaporated under reduced pressure. The residue was dissolved in DCM and adsorbed on a silica gel pre-column and purified by silica gel flash column chromatography (120 g), eluting with a 100% heptanes to 30% ethyl acetate-heptanes gradient. The product fractions were combined and evaporated under reduced pressure to afford methyl 2-((2-(((tert-butoxycarbonyl)(2-(6-methoxy-3-nitropyridin-2-yl)ethyl)amino)methyl)-4-fluorophenyl)amino)-5-fluoro-4-(trifluoromethyl)benzoate 44b as a clear oil, HPLC/MS 1.64 min (B), [M+H]⁺ 641.2. ¹H NMR (CDCl₃, 400 MHz) δ 9.05 (s, 1H), 8.20 (d, J=8.8 Hz, 1H), 7.77 (br d, J=10.8 Hz, 1H), 7.22 (dd, J=8.6, 5.14 Hz, 1H), 7.00-7.16 (m, 2H), 6.80 (br s, 1H), 6.65 (br d, J=8.8 Hz, 1H), 4.47 (br s, 2H), 3.93-3.98 (m, 6H), 3.69-3.80 (m, 1H), 3.65 (br s, 1H), 3.28 (br s, 2H), 1.37-1.42 (m, 9H).

### Step C: 2-((2-(((tert-Butoxycarbonyl)(2-(6-methoxy-3-nitropyridin-2-yl)ethyl)amino)-methyl)-4-fluoro phenyl)amino)-5-fluoro-4-(trifluoromethyl)benzoic acid 44c

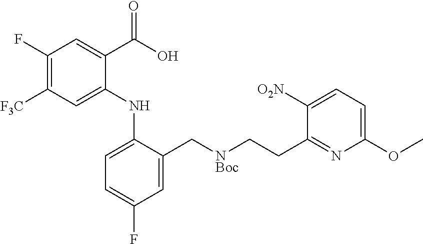 [SMILES: COc1ccc([N+](=O)[O-])c(CCN(Cc2cc(F)ccc2Nc2cc(C(F)(F)F)c(F)cc2C(=O)O)C(=O)OC(C)(C)C)n1]

To a mixture of methyl 2-((2-(((tert-butoxycarbonyl)(2-(6-methoxy-3-nitropyridin-2-yl)ethyl)amino)methyl)-4-fluorophenyl)amino)-5-fluoro-4-(trifluoromethyl)benzoate (0.920 g, 1.44 mmol) in H₂O (5 ml), MeOH (5 ml), and THF (5 ml), was added lithium hydroxide (275.2 mg, 11.5 mmol), and the reaction mixture was stirred for 4 h. The reaction mixture was neutralized with 1N HCl, the residue partitioned into EtOAc and H₂O, the layers separated, and the aqueous phase extracted with EtOAc. The combined organic extracts were washed with brine, dried over MgSO₄, filtered and the solvent evaporated under reduced pressure to afford 2-((2-(((tert-butoxycarbonyl)(2-(6-methoxy-3-nitropyridin-2-yl)ethyl)amino)methyl)-4-fluorophenyl)amino)-5-fluoro-4-(trifluoromethyl)benzoic acid 44c (950 mg, 100% yield) as a light yellow oil. HPLC/MS 1.08 min (B), [M+H]⁺ 627.2. ¹H NMR (CDCl₃, 400 MHz) δ 9.03 (br s, 1H), 8.10 (br d, J=8.80 Hz, 1H), 7.62 (br s, 1H), 7.08 (br s, 1H), 6.89 (br s, 2H), 6.71 (br s, 1H), 6.56 (br d, J=8.80 Hz, 1H), 4.52 (br s, 2H), 3.83 (s, 3H), 3.64 (br t, J=6.60 Hz, 2H), 3.22 (t, J=6.85 Hz, 2H), 1.34 (br s, 9H).

### Step D: 2-((2-(((2-(3-Amino-6-methoxypyridin-2-yl)ethyl)(tert-butoxycarbonyl)amino)methyl)-4-fluoro phenyl)amino)-5-fluoro-4-(trifluoromethyl)benzoic acid 44d

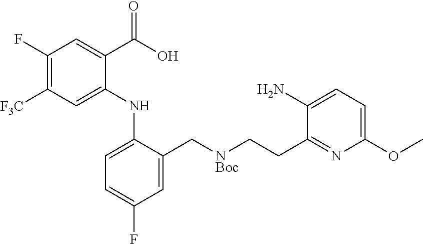 [SMILES: COc1ccc(N)c(CCN(Cc2cc(F)ccc2Nc2cc(C(F)(F)F)c(F)cc2C(=O)O)C(=O)OC(C)(C)C)n1]

A suspension of 2-((2-(((tert-butoxycarbonyl)(2-(6-methoxy-3-nitropyridin-2-yl)ethyl)amino)methyl)-4-fluorophenyl)amino)-5-fluoro-4-(trifluoromethyl)benzoic acid (890 mg, 1.42 mmol), in MeOH (10 mL), was sparged with N₂ (3×), to which was added 10% Pd—C (151.2 mg, 0.142 mmol), the suspension purged with N₂ (3×), a balloon of H₂ was added, the reaction sparged (4×) and allowed to stir at room temperature for 18.5 h The catalyst was filtered through a plug of celite and the solvent evaporated under reduced pressure to afford 2-((2-(((2-(3-amino-6-methoxypyridin-2-yl)ethyl)(tert-butoxycarbonyl)amino)methyl)-4-fluorophenyl)amino)-5-fluoro-4-(trifluoromethyl)benzoic acid 44d (846 mg, 94% yield) as a brown solid, HPLC/MS 1.01 min (B), [M+H]'⁰ 597.1. ^{t}H NMR (CDCl₃, 400 MHz) δ 8.89 (br s, 1H), 7.70 (br s, 1H), 7.41-7.57 (m, 1H), 7.01 (br s, 1H), 6.87-6.96 (m, 1H), 6.84 (br s, 1H), 6.62 (br s, 1H), 6.39 (br d, J=1.0 Hz, 1H), 4.30 (br s, 2H), 3.65 (br s, 3H), 3.50 (br s, 2H), 3.06 (br s, 2H), 1.19-1.37 (m, 9H).

### Step E: tert-Butyl 2,8-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 44e

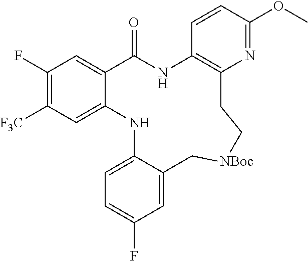 [SMILES: COc1ccc2c(n1)CCN(C(=O)OC(C)(C)C)Cc1cc(F)ccc1Nc1cc(C(F)(F)F)c(F)cc1C(=O)N2]

To a solution of 2-((2-(((2-(3-amino-6-methoxypyridin-2-yl)ethyl)(tert-butoxycarbonyl)amino)methyl)-4-fluorophenyl)amino)-5-fluoro-4-(trifluoromethyl)benzoic acid (800 mg, 1.34 mmol) in DMF (10 mL) was added pyoxim (1.37 g, 2.68 mmol) followed by 4-methylmorpholine (0.59 mL, 5.36 mmol) and the reaction mixture was stirred at room temperature for 1 h. The crude reaction was filtered through a 0.2 μm acrodisc, and purified by C18 revese phase semi-prep HPLC (100 g) eluting at 60 mL/min with a 40% CH₃CN/H₂O (pH10) to 100% CH₃CN/H₂O. Pure product fractions were combined and evaporated under reduced pressure to afford tert-butyl 2,8-difluoro-15-methoxy-19-oxo-3-(trifluoromethyl)-5,10,12,13,18,19-hexahydro-11H-dibenzo[c,f]pyrido[3,27][1,5,9]triazacyclotridecine-11-carboxylate 44e (429 ng, 55% yield) as a beige solid. HPLC/MS 1.48 min (B), [M+H]⁺ 579.1 ¹H NMR (DMSO-d₆, 400 MHz) δ ppm 7.83 (br d, J=7.3 Hz, 2H), 7.59 (s, 1H), 7.34-7.50 (m, 1H), 7.18 (br d, J=9.8 Hz, 1H), 7.01 (br d, J=2.9 Hz, 1H), 6.71 (d, J=8.3 Hz, 1H), 6.53-6.68 (m, 1H), 4.58 (s, 2H), 3.81 (s, 3H), 3.33-3.41 (m, 2H), 2.54-2.75 (m, 2H), 1.24-1.63 (m, 9H).

### Step F tert-Butyl 2,8-difluoro-15-methoxy-19-oxo-3-(trifluoromethyl)-12,13-dihydro-19H-5,18-methano dibenzo[c,f]pyrido[3,2-j][1,5,9]triazacyclotridecine-11(10H)-carboxylate 44f

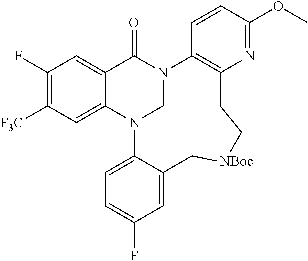 [SMILES: COc1ccc2c(n1)CCN(C(=O)OC(C)(C)C)Cc1cc(F)ccc1N1CN2C(=O)c2cc(F)c(C(F)(F)F)cc21]

To a 100 mL round bottomed flask was added a solution of tert-butyl 2,8-difluoro-15-methoxy-19-oxo-3-(trifluoromethyl)-5,10,12,13,18,19-hexahydro-11-dibenzo[c,f]pyrido[3,2-j][1,5,9]triazacyclotridecine-11-carboxylate (429 mg, 0.742 mmol), and Cs₂CO₃ (1.45 g, 4.45 mmol) followed by MeCN (20 mL) and diiodomethane (897 μL, 11.1 mmol), and the reaction mixture was stirred for 30 min at room temperature and then heated to 90° C. for 23 h. The reaction mixture was cooled to ambient temperature, filtered, washed with DCM and MeCN and the solution evaporated under reduced pressure. The crude product was dissolved in DMSO and purified by C18 revese-phase semi-prep HPLC (100 g) eluting at 60 mL/min with a 40% CH₃CN/H₂ (pH10) to 100% CH₃CN/H₂O gradient. Pure product fractions were combined and evaporated under reduced pressure to afford ter-butyl 2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 44f (309 mg, 71% yield) as a light tan solid. HPLC/MS 1.48 min (B), [M+H]⁺ 591.1 ¹H NMR (DMSO-d₆, 400 MHz) δ ppm 7.92 (d, J=10.8 Hz, 1H), 7.75 (d, J=8.8 Hz, 1H), 7.55 (dd, J=8.8, 5.38 Hz, 1H), 7.28-7.45 (m, 2H), 6.82 (d, J=8.8 Hz, 1H), 6.45 (d, J=5.4 Hz, 1H), 5.64 (d, J=11.7 Hz, 1H), 4.91 (d, J=11.7 Hz, 1H), 4.73 (d, J=14.7 Hz, 1H), 3.98 (br d, J=14.2 Hz, 1H), 3.88 (s, 3H), 3.54-3.80 (m, 1H), 3.01 (td, J=11.3, 2.9 Hz, 1H), 2.65-2.83 (m, 1H), 2.55-2.65 (m, 1H), 1.46 (s, 9H).

### Step G: 2,8-Difluoro-3-(trifluoromethyl)-10,11,12,13-tetrahydro-19H-5,18-methanodibenzo[c,f]pyrido[3,2-j][1,5,9]triazacyclotridecine-15,19(14H)-dione, hydrochloride salt

### Example 44

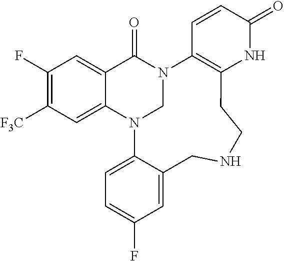 [SMILES: O=C1c2cc(F)c(C(F)(F)F)cc2N2CN1c1ccc(=O)[nH]c1CCNCc1cc(F)ccc12]

To tert-butyl 2,8-difluoro-15-methoxy-19-oxo-3-(trifluoromethyl)-12,13-dihydro-19H-5,18-methano dibenzo[c,f]pyrido[3,2-j][1,5,9]triazacyclotridecine-11(10H)-carboxylate (309 mg, 0.523 mmol), in a 20 ml, microwave vial, was added 5 NHCl in isopropanol (5.23 mL, 26.2 mmol) and the reaction mixture was stirred at 90° C. for 20.5 b. The reaction mixture was evaporated under reduced pressure, triturated with MeOH, filtered and dried to afford 2,8-difluoro-3-(trifluoromethyl)-10,11,12,13-tetrahydro-19H-5,18-methanodibenzo[c,f]pyrido[3,2-j][1,5,9]-triazacyclotridecine-15,19(14H)-dione, hydrochloride salt (175 mg, 70% yield) as a white solid. HPLC/MS 1.03 min (B), [M+H]⁺ 477.1 ¹H NMR (DMSO-d₆, 400 MHz) δ 10.61-10.88 (m, 1H), 8.82-9.09 (m, 1H), 7.91 (d, J=10.8 Hz, 1H), 7.79 (dd, J=9.3, 2.9 Hz, 1H), 7.66 (dd, J=8.8, 5.4 Hz, 1H) 7.37-7.58 (m, 2H), 6.44 (d, J=5.4 Hz, 1H), 6.35 (d, J=9.8 Hz, 1H), 5.46 (d, J=11.7 Hz, 1H), 4.97 (d, J=11.7 Hz, 1H), 4.11-4.24 (m, 1H), 3.96-4.08 (m, 1H), 3.12-3.19 (m, 1H), 2.94-3.12 (m, 2H), 2.60-2.78 (m, 1H).

### Example 45

### 1,8-Difluoro-2-(trifluoromethyl)-10,11,12,13-tetrahydro-19H-5,18-methanodibenzo[c,f]pyrido[3,2-j][1,5,9]triazacyclotridecine-15,19(14H)-dione

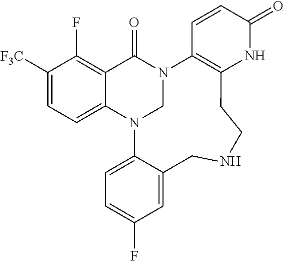 [SMILES: O=C1c2c(ccc(C(F)(F)F)c2F)N2CN1c1ccc(=O)[nH]c1CCNCc1cc(F)ccc12]

### Step A: Methyl 2-fluoro-6-((4-fluoro-2-formylphenyl)amino)-3-(trifluoromethyl)-benzoate 45a

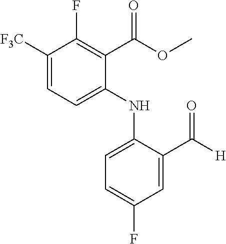 [SMILES: COC(=O)c1c(Nc2ccc(F)cc2C=O)ccc(C(F)(F)F)c1F]

Following the preparation outlined in Example 7, Step A, substituting ethyl 2-bromo-5-(trifluoromethyl)benzoate with methyl 6-amino-2-fluoro-3-(trifluoromethyl)benzoate Int-2g and 2-bromo-5-fluorobenzaldehyde with 2-(but-3-en-1-yl)-4-fluoroaniline Int-3a, BINAP with Xantphos, toluene with 1,4-dioxane, and stirring the reaction mixture at 80° C. overnight, methyl 2-fluoro-6-((4-fluoro-2-formylphenyl)amino)-3-(trifluoromethyl)benzoate 45a (375 mg, 1.04 mmol, 57% yield) was prepared as a yellow solid. HPLC/MS 1.28 min (B), [M+H]⁺ 359.9. ¹H NMR (CDCl₃, 400 MHz) δ 3.85 (s, 3H) 6.97-7.14 (m, 2H) 7.24-7.49 (m, 3H) 9.81 (s, 1H) 10.56 (br s, 1H).

### Step B: Methyl 6-((2-(((tert-butoxycarbonyl)(2-(6-methoxy-3-nitropyridin-2-yl)ethyl)-amino)methyl)-4-fluorophenyl)amino)-2-fluoro-3-(trifluoromethyl)benzoate 45b

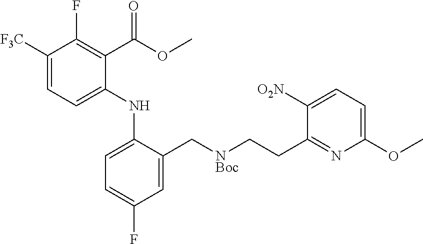 [SMILES: COC(=O)c1c(Nc2ccc(F)cc2CN(CCc2nc(OC)ccc2[N+](=O)[O-])C(=O)OC(C)(C)C)ccc(C(F)(F)F)c1F]

Following the preparation outlined in Example 44, Step B, substituting methyl 5-fluoro-2-((4-fluoro-2-formylphenyl)amino)-4-(trifluoromethyl)benzoate with methyl 2-fluoro-6-((4-fluoro-2-formylphenyl)amino)-3-(trifluoromethyl)benzoate 45a, methyl 6-((2-(((tert-butoxy-carbonyl)(2-(6-methoxy-3-nitropyridin-2-yl)ethyl)amino)methyl)-4-fluorophenyl)amino)-2-fluoro-3-(trifluoromethyl)benzoate 45b (1.27 g, 71% yield) was prepared as a clear oil. HPLC/MS 1.62 min (B), [M+H]⁺ 641.0. ¹H NMR (CDCl₃, 400 MHz) δ 1.36 (s, 11H) 3.25 (br s, 2H) 3.64 (br s, 2H) 3.91 (s, 4H) 3.96 (s, 4H) 4.36 (br s, 2H) 6.26-6.33 (m, 1H) 6.62 (br d, J=8.80 Hz, 1H) 7.00 (td, J=8.07, 2.93 Hz, 1H) 7.06 (br d, J=8.31 Hz, 1H) 7.18 (dd, J=8.56, 5.14 Hz, 1H) 7.27-7.34 (m, 1H) 8.15 (d, J=9.29 Hz, 1H) 9.26 (s, 1H).

### Step C: 6-((2-(((tert-butoxycarbonyl)(2-(6-methoxy-3-nitropyridin-2-yl)ethyl)amino)-methyl)-4-fluorophenyl)amino)-2-fluoro-3-(trifluoromethyl)benzoic acid 45c

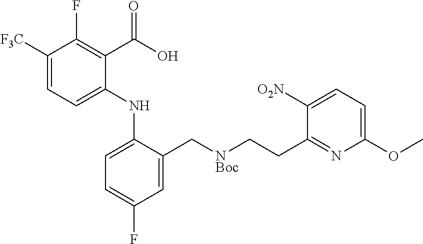 [SMILES: COc1ccc([N+](=O)[O-])c(CCN(Cc2cc(F)ccc2Nc2ccc(C(F)(F)F)c(F)c2C(=O)O)C(=O)OC(C)(C)C)n1]

Following the procedure outlined in Example 41, Step C, stirring the reaction mixture at 50° C. for 3 h, 6-((2-(((tert-butoxycarbonyl)(2-(6-methoxy-3-nitropyridin-2-yl)ethyl)amino)-methyl)-4-fluorophenyl)amino)-2-fluoro-3-(trifluoromethyl)benzoic acid 45c (1.18 g, 95% yield) was prepared as a light yellow powder. HPLC/MS 1.07 min (B), [M+H]⁺ 627.1. ¹H NMR (CDCl₃, 400 MHz) δ 1.34-1.54 (m, 9H) 3.22-3.39 (m, 2H) 3.68 (br s, 2H) 3.95 (s, 3H) 4.47 (br s, 2H) 6.28 (d, J=9.29 Hz, 1H) 6.66 (br d, J=8.80 Hz, 1H) 7.06 (br d, J=8.80 Hz, 2H) 7.14-7.23 (m, 1H) 7.33-7.40 (m, 1H) 8.19 (d, J=9.29 Hz, 1H) 9.44 (br s, 1H).

### Step D: 6-((2-(((2-(3-amino-6-methoxypyridin-2-yl)ethyl)(tert-butoxycarbonyl)amino)-methyl)-4-fluorophenyl)amino)-2-fluoro-3-(trifluoromethyl)benzoic acid 45d

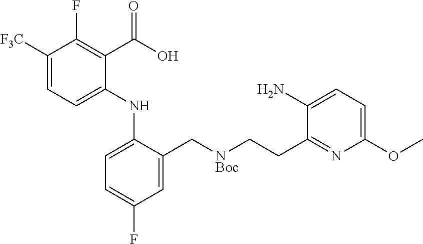 [SMILES: COc1ccc(N)c(CCN(Cc2cc(F)ccc2Nc2ccc(C(F)(F)F)c(F)c2C(=O)O)C(=O)OC(C)(C)C)n1]

Following the procedure outlined in Example 44, Step D, 6-((2-(((2-(3-amino-6-methoxypyridin-2-yl)ethyl)(tert-butoxycarbonyl)amino)methyl)-4-fluorophenyl)amino)-2-fluoro-3-(trifluoromethyl)benzoic acid 45d (1.16 g, 100% yield) was prepared as a gray solid. HPLC/MS 0.96 min (B), [M+H]⁺ 597.1. ¹H NMR (CDCl₃, 400 MHz) δ 1.44 (s, 9H) 2.91 (br s, 2H) 3.44-3.54 (m, 2H) 3.77 (s, 3H) 4.35 (s, 2H) 6.27 (br d, J=8.80 Hz, 1H) 6.47 (br d, J=8.80 Hz, 1H) 6.95 (br t, J=6.60 Hz, 1H) 7.06 (dd, J=9.05, 2.69 Hz, 1H) 7.09-7.18 (m, 2H) 7.23-7.29 (m, 1H).

### Step E: tert-Butyl 1,8-difluoro-15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 45e

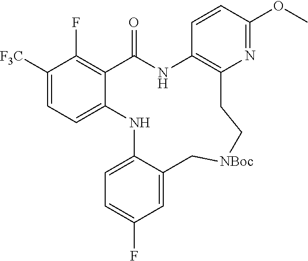 [SMILES: COc1ccc2c(n1)CCN(C(=O)OC(C)(C)C)Cc1cc(F)ccc1Nc1ccc(C(F)(F)F)c(F)c1C(=O)N2]

Following the procedure outlined in Example 41, Step E, substituting HATU with pyoxim and stirring the reaction mixture at RT for 2 h, tert-butyl 1,8-difluoro-15-methoxy-19-oxo-2-(trifluoromethyl)-5,10,12,13,18,19-hexahydro-11H-dibenzo[c,f]pyrido[3,2-j][1,5,9]triazacyclo-tridecine-11-carboxylate 45e (463 mg, 58% yield) was prepared as a white solid. HPLC/MS 1.45 min (B), [M+H]⁺ 579.1. ¹H NMR (CD₃OD, 400 MHz) δ 1.56 (s, 9H), 2.90-3.05 (m, 2H), 3.39-3.58 (m, 2H), 3.82-3.99 (m, 3H), 4.42 (s, 2H), 6.44 (d, J=8.80 Hz, 1H), 6.72 (d, J=8.80 Hz, 1H), 7.04-7.17 (m, 1H), 7.22 (dd, J=8.56, 5.14 Hz, 1H), 7.42 (dd, J=9.29, 2.93 Hz, 1H), 7.54 (t, J=8.56 Hz, 1H), 7.86 (d, J=8.80 Hz, 1H).

### Step F: tert-Butyl 1,8-difluoro-15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate 45f

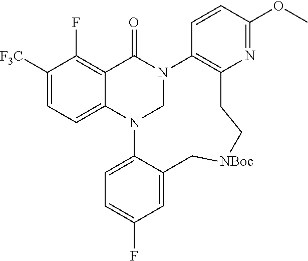 [SMILES: COc1ccc2c(n1)CCN(C(=O)OC(C)(C)C)Cc1cc(F)ccc1N1CN2C(=O)c2c1ccc(C(F)(F)F)c2F]

Following the procedure outlined in Example 41, Step F, stirring the reaction at 90° C. overnight, tert-butyl 1,8-difluoro-15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate 45f (17 mg, 2% yield) was prepared as a white solid. HPLC/MS 1.43 min (B), [M+H]⁺ 591.1. ¹H NMR (CD₃OD, 400 MHz) δ 7.60-7.72 (m, 2H), 7.34-7.46 (m, 2H), 7.23-7.31 (m, 1H), 6.77 (d, J=8.80 Hz, 1H), 6.27 (d, J=8.80 Hz, 1H), 5.65 (d, J=11.74 Hz, 1H), 4.01-4.08 (m, 1H), 3.89-3.96 (m, 3H), 3.81-3.88 (m, 1H), 3.37-3.46 (m, 2H), 3.02-3.11 (m, 1H), 2.71-2.77 (m, 1H), 1.45-1.61 (m, 9H).

### Step G: 1,8-Difluoro-2-(trifluoromethyl)-10,11,12,13-tetrahydro-19H-5,18-methano-dibenzo[c,f]pyrido[3,2-j][1,5,9]triazacyclotridecine-15,19(14H)-dione Example 45

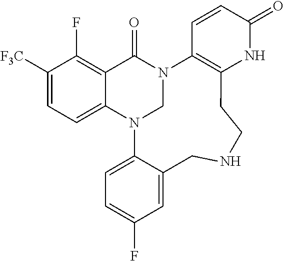 [SMILES: O=C1c2c(ccc(C(F)(F)F)c2F)N2CN1c1ccc(=O)[nH]c1CCNCc1cc(F)ccc12]

Following the procedure outlined in Example 41, Step G, stirring the reaction mixture at 90° C. for 3 h, 1,8-difluoro-2-(trifluoromethyl)-10,11,12,13-tetrahydro-19H-5,18-methano-dibenzo[c,f]pyrido[3,2-j][1,5,9]triazacyclotridecine-15,19(14H)-dione (5 mg, 36% yield) was prepared as a white solid. HPLC/MS 0.98 min (B), [M+H]⁺ 477.1. ¹H NMR (CD₃OD, 400 MHz) δ 7.57 (dd, J=8.80, 7.83 Hz, 1H), 7.53 (d, J=9.29 Hz, 1H), 7.45 (dd, J=8.56, 5.14 Hz, 1H), 7.36 (dd, J=8.80, 2.93 Hz, 1H), 7.19-7.27 (m, 1H), 6.44 (d, J=9.29 Hz, 1H), 6.34 (d, J=8.80 Hz, 1H), 5.43 (d, J=9.78 Hz, 1H,) 5.11-5.17 (m, 1H), 3.95 (d, J=11.25 Hz, 1H), 3.44-3.52 (m, 1H), 2.96-3.10 (m, 3H), 2.33-2.40 (m, 1H).

### Example 46

### 3-Chloro-2,8-difluoro-10,11,12,13-tetrahydro-19H-5,18-methanodibenzo[c,f]pyrido[3,2-j][1,5,9]triazacyclotridecine-15,19(14H)-dione, hydrochloride

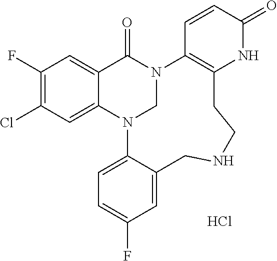 [SMILES: O=C1c2cc(F)c(Cl)cc2N2CN1c1ccc(=O)[nH]c1CCNCc1cc(F)ccc12 ; Cl]

### Step A: Methyl 4-chloro-5-fluoro-2-((4-fluoro-2-formylphenyl)amino)benzoate 46a

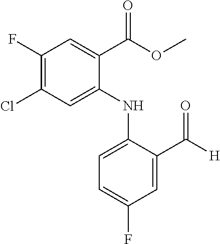 [SMILES: COC(=O)c1cc(F)c(Cl)cc1Nc1ccc(F)cc1C=O]

Following the preparation outlined in Example 7, Step A, substituting ethyl 2-bromo-5-(trifluoromethyl)benzoate with methyl 2-amino-4-chloro-5-fluorobenzoate and 2-bromo-5-fluorobenzaldehyde with 2-(but-3-en-1-yl)-4-fluoroaniline Int-3a, BINAP with Xantphos, toluene with 1,4-dioxane, and stirring the reaction mixture at 100° C. for 18 h, methyl 4-chloro-5-fluoro-2-((4-fluoro-2-formylphenyl)amino)benzoate 46a (3.85 g, 48% yield) was prepared as a bright yellow solid. HPLC/MS 1.27 min (A), [M+H]⁺ 326.8. ¹H NMR (DMSO-d₆, 400 MHz): δ 9.96 (s, 1H), 7.86 (d, J=9.8 Hz, 1H), 7.76 (dd, J=8.3, 2.9 Hz, 1H), 7.61 (d, J=6.8 Hz, 1H), 7.45-7.51 (m, 2H), 3.88 (s, 3H).

### Step B: Methyl 2-((2-(((tert-butoxycarbonyl)(2-(6-methoxy-3-nitropyridin-2-yl)ethyl-amino)methyl)-4-fluorophenyl)amino)-4-chloro-5-fluorobenzoate 46b

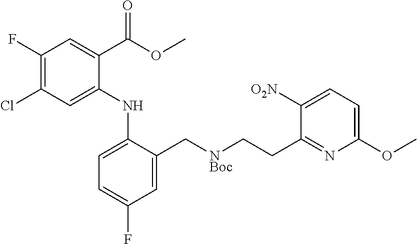 [SMILES: COC(=O)c1cc(F)c(Cl)cc1Nc1ccc(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4-chloro-5-fluoro-2-((4-fluoro-2-formylphenyl)amino)benzoate 46a, methyl 2-((2-(((tert-butoxycarbonyl)(2-(6-methoxy-3-nitropyridin-2-yl)ethyl)amino)methyl)-4-fluorophenyl)amino)-4-chloro-5-fluoro-benzoate 46b (69 mg, 34% yield) was prepared as an oil. HPLC/MS 1.62 min (A), [M+H]⁺ 607.2. ¹H NMR (DMSO-d₆, 400 MHz): δ 8.85 (s, 1H), 8.30 (d, J=8.8 Hz, 1H), 7.77 (d, J=10.3 Hz, 1H), 7.34 (dd, J=8.8, 5.4 Hz, 1H), 7.21 (td, J=8.3, 2.9 Hz, 1H), 7.01-7.13 (m, 1H), 6.85 (br d, J=6.4 Hz, 1H), 4.30 (br s, 2H), 3.88 (s, 3H), 3.86 (s, 3H), 3.62 (br s, 2H), 3.15 (br s, 2H), 1.21 (s, 9H).

### Step C: 2-((2-(((tert-Butoxycarbonyl)(2-(6-methoxy-3-nitropyridin-2-yl)ethyl)amino)-methyl)-4-fluorophenyl)amino)-4-chloro-5-fluorobenzoic acid 46c

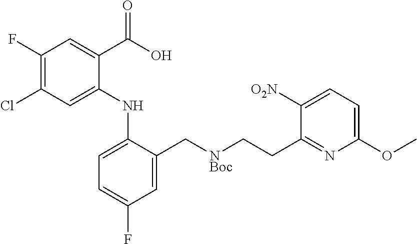 [SMILES: COc1ccc([N+](=O)[O-])c(CCN(Cc2cc(F)ccc2Nc2cc(Cl)c(F)cc2C(=O)O)C(=O)OC(C)(C)C)n1]

Following the procedure outlined in Example 41, Step C, stirring the reaction mixture at ambient temperature for 44 h, 2-((2-(((tert-butoxycarbonyl)(2-(6-methoxy-3-nitropyridin-2-yl)ethyl)amino)methyl)-4-fluorophenyl)amino)-4-chloro-5-fluorobenzoic acid 46c (68 mg, 101% yield) was prepared as a yellow oil. HPLC/MS 1.51 min (A), [M+H]⁺ 593.2. ¹H NMR (DMSO-d₆, 400 MHz): δ 9.17 (br s, 1H), 8.30 (br d, J=8.8 Hz, 1H), 7.75 (d, J=9.8 Hz, 1H), 7.35 (dd, J=8.6, 5.1 Hz, 1H), 7.20 (td, J=8.4, 3.2 Hz, 1H), 7.06 (br d, J=2.4 Hz, 1H), 6.85 (br d, J=5.9 Hz, 1H), 4.32 (br s, 2H), 3.86 (s, 3H), 3.57-3.70 (m, 2H), 3.16 (br d, J=2.9 Hz, 2H), 1.18-1.23 (m, 9H).

### Step D: 2-((2-(((2-(3-amino-6-methoxypyridin-2-yl)ethyl)(tert-butoxycarbonyl)amino)-methyl)-4-fluorophenyl)amino)-4-chloro-5-fluorobenzoic acid 46d

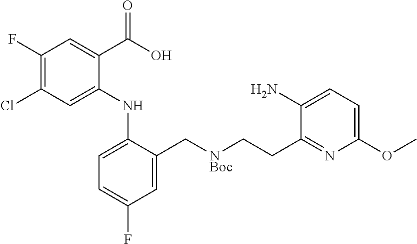 [SMILES: COc1ccc(N)c(CCN(Cc2cc(F)ccc2Nc2cc(Cl)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-fluorophenyl)amino)-4-chloro-5-fluorobenzoic acid 46d (1.16 g, 100% yield) was prepared as a gray solid. HPLC/MS 0.96 min (B), [M+H]⁺ 597.1. ¹H NMR (CDCl₃, 400 MHz): δ 1.44 (s, 9H) 2.91 (br s, 2H) 3.44-3.54 (m, 2H) 3.77 (s, 3H) 4.35 (s, 2H) 6.27 (br d, J=8.80 Hz, 1H) 6.47 (br d, J=8.80 Hz, 1H) 6.95 (br t, J=6.60 Hz, 1H) 7.06 (dd, J=9.05, 2.69 Hz, 1H) 7.09-7.18 (m, 2H) 7.23-7.29 (m, 1H).

### Step E: tert-Butyl 3-chloro-2,8-difluoro-15-methoxy-19-oxo-5,10,12,13,18,19-hexa-hydro-11H-dibenzo[c,f]pyrido[3,2-j][1,5,9]triazacyclotridecine-11-carboxylate 46e

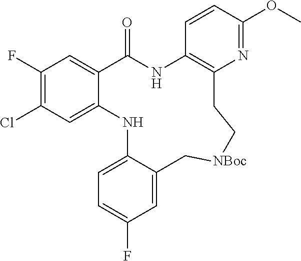 [SMILES: COc1ccc2c(n1)CCN(C(=O)OC(C)(C)C)Cc1cc(F)ccc1Nc1cc(Cl)c(F)cc1C(=O)N2]

Following the procedure outlined in Example 41, Step E, substituting HATU with pyoxim and stirring the reaction mixture at RT for 18 h, tert-butyl 3-chloro-2,8-difluoro-15-methoxy-19-oxo-5,10,12,13,18,19-hexahydro-11H-dibenzo[c,f]pyrido[3,2-j][1,5,9]triazacyclotridecine-11-carboxylate 46e (26 mg, 51% yield) was prepared. HPLC/MS 1.37 min (A), [M+H]⁺ 545.2. ¹H NMR (DMSO-d₆, 400 MHz): δ 7.88 (br d, J=8.8 Hz, 1H), 7.80 (br s, 1H), 7.52 (s, 1H), 7.21 (br d, J=6.4 Hz, 1H), 7.00 (td, J=8.4, 3.2 Hz, 1H), 6.70 (d, J=8.8 Hz, 1H), 6.46-6.62 (m, 1H), 4.59 (s, 2H), 3.81 (s, 3H), 3.33-3.43 (m, 4H), 1.26-1.55 (m, 9H).

### Step F: tert-Butyl 3-chloro-2,8-difluoro-15-methoxy-19-oxo-12,13-dihydro-19H-5,18-methanodibenzo[c,f]pyrido[3,2-j][1,5,9]triazacyclotridecine-11(10H)-carboxylate 46f

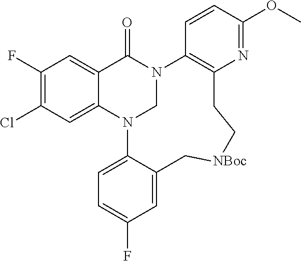 [SMILES: COc1ccc2c(n1)CCN(C(=O)OC(C)(C)C)Cc1cc(F)ccc1N1CN2C(=O)c2cc(F)c(Cl)cc21]

Following the procedure outlined in Example 41, Step F, stirring the reaction at 90° C. for 22 h, tert-butyl 3-chloro-2,8-difluoro-15-methoxy-19-oxo-12,13-dihydro-19H-5,18-methano-dibenzo[c,f]pyrido[3,2-j][1,5,9]triazacyclotridecine-11(10H)-carboxylate 46f (210 mg, 76% yield) was prepared. HPLC/MS 1.40 min (A), [M+H]⁺ 557.2. ¹H NMR (DMSO-d₆, 400 MHz): δ 7.80 (d, J=9.3 Hz, 1H), 7.71 (d, J=8.3 Hz, 1H), 7.49 (dd, J=8.8, 5.4 Hz, 1H), 7.37 (td, J=8.3, 2.9 Hz, 1H), 7.30 (dd, J=9.0, 3.2 Hz, 1H), 6.76-6.81 (m, 1H), 6.36 (d, J=5.9 Hz, 1H), 5.59 (d, J=11.7 Hz, 2H), 4.85 (d, J=11.7 Hz, 2H), 4.72 (d, J=14.2 Hz, 2H), 3.93 (br d, J=14.2 Hz, 2H), 3.85 (s, 3H), 1.45 (s, 9H).

### Step G: 3-Chloro-2,8-difluoro-10,11,12,13-tetrahydro-19H-5,18-methanodibenzo[c,f]pyrido[3,2-j][1,5,9]triazacyclotridecine-15,19(14H)-dione, hydrochloride Example 46

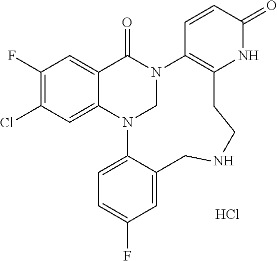 [SMILES: O=C1c2cc(F)c(Cl)cc2N2CN1c1ccc(=O)[nH]c1CCNCc1cc(F)ccc12 ; Cl]

Following the procedure outlined in Example 41, Step G, stirring the reaction mixture at 90° C. for 8 h, 3-chloro-2,8-difluoro-10,11,12,13-tetrahydro-19H-5,18-methanodibenzo[c,]pyrido[3,2-j][1,5,9]triazacyclotridecine-15,19(14H)-dione, hydrochloride (170 mg, 95% yield) was prepared. HPLC/MS 0.66 min (A), [M+H]⁺ 443.2. ¹H NMR (DMSO-d₆, 400 MHz): δ 10.53 (br s, 1H), 8.86-8.98 (m, 1H), 7.82 (d, J=9.29 Hz, 1H), 7.75 (dd, J=9.29, 2.93 Hz, 1H), 7.61 (dd, J=8.56, 5.62 Hz, 1H), 7.47-7.54 (m, 1H), 7.45 (d, J=9.78 Hz, 1H), 6.31-6.39 (m, 2H), 5.45 (d, J=11.25 Hz, 1H), 4.91 (d, J=11.25 Hz, 1H), 4.12 (br s, 2H), 3.14-3.23 (m, 1H), 2.95-3.06 (m, 2H), 2.62-2.70 (m, 1H).

### Example 47

### 7,8-Difluoro-2-(trifluoromethyl)-10,11,12,13-tetrahydro-19H-5,18-methano-dibenzo[c,f]pyrido[3,2-j][1,5,9]triazacyclotridecine-15,19(14H)-dione hydrochloride

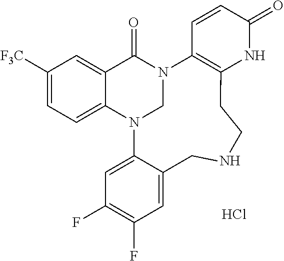 [SMILES: O=C1c2cc(C(F)(F)F)ccc2N2CN1c1ccc(=O)[nH]c1CCNCc1cc(F)c(F)cc12 ; Cl]

### Step A: Methyl 2-((4,5-difluoro-2-formylphenyl)amino)-5-(trifluoromethyl)benzoate 47a

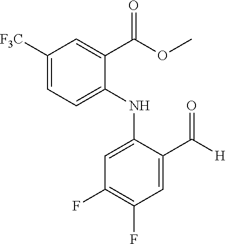 [SMILES: COC(=O)c1cc(C(F)(F)F)ccc1Nc1cc(F)c(F)cc1C=O]

Following the preparation outlined in Example 7, Step A, substituting ethyl 2-bromo-5-(trifluoromethyl)benzoate with methyl 2-bromo-5-(trifluoromethyl)benzoate and Int-3a with 2-amino-4,5-difluorobenzaldehyde, toluene with 1,4-dioxane, and stirring the reaction at 80° C. for 4.5 h, methyl 2-((4,5-difluoro-2-formylphenyl)amino)-5-(trifluoromethyl)benzoate 47a (1.17 g, 45% yield) was prepared as a light grey solid. HPLC/MS 1.34 min (A), [M+H]⁺ 359.1. ¹H NMR (DMSO-d₆, 400 MHz): δ 11.16 (s, 1H), 9.93 (s, 1H), 8.19 (s, 1H), 8.06 (s, 1H), 7.84 (br d, 1H, J=8.8 Hz), 7.6-7.8 (m, 2H), 3.92 (s, 3H).

### Step B: Methyl 2-((2-(((tert-butoxycarbonyl)(2-(6-methoxy-3-nitropyridin-2-yl)ethyl-amino)methyl)-4,5-difluorophenyl)amino)-5-(trifluoromethyl)benzoate 47b

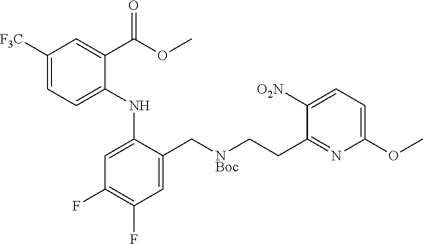 [SMILES: COC(=O)c1cc(C(F)(F)F)ccc1Nc1cc(F)c(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((4,5-difluoro-2-formylphenyl)amino)-5-(trifluoromethyl)benzoate 47a, methyl 2-((2-(((tert-butoxycarbonyl)(2-(6-methoxy-3-nitropyridin-2-yl)ethyl)amino)methyl)-4,5-difluorophenyl)amino)-5-(trifluoro-methyl)benzoate 47b (2.08 g, 76% yield) was prepared as an off white foam. HPLC/MS 1.60 min (A), [M+H]⁺ 641.2. ¹H NMR (DMSO-d₆, 400 MHz): δ 9.2-9.5 (m, 1H), 8.2-8.3 (m, 1H), 8.1-8.2 (m, 1H), 7.6-7.7 (m, 1H), 7.5-7.5 (m, 1H), 7.2-7.4 (m, 1H), 6.8-6.9 (m, 1H), 6.6-6.8 (m, 1H), 4.31 (brs, 2H), 3.90 (s, 3H), 3.84 (s, 3H), 3.5-3.7 (m, 2H), 3.13 (br s, 2H), 1.18 (s, 10H).

### Step C: 2-((2-(((tert-Butoxycarbonyl)(2-(6-methoxy-3-nitropyridin-2-yl)ethyl)amino)-methyl)-4,5-difluorophenyl)amino)-5-(trifluoromethyl)benzoic acid 47c

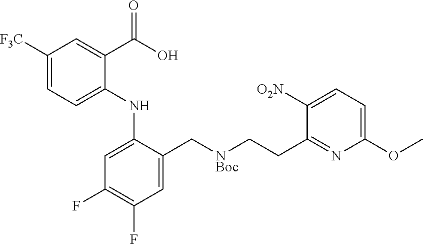 [SMILES: COc1ccc([N+](=O)[O-])c(CCN(Cc2cc(F)c(F)cc2Nc2ccc(C(F)(F)F)cc2C(=O)O)C(=O)OC(C)(C)C)n1]

Following the procedure outlined in Example 41, Step C, stirring the reaction mixture at 50° C. for 4.5 h, 2-((2-(((tert-butoxycarbonyl)(2-(6-methoxy-3-nitropyridin-2-yl)ethyl)amino)-methyl)-4,5-difluorophenyl)amino)-5-(trifluoromethyl)benzoic acid 47c (2.02 g, 96% yield) was prepared as a light yellow foam. HPLC/MS 1.50 min (A), [M+H]⁺ 627.2. ¹H NMR (DMSO-d₆, 400 MHz): δ 13.3-13.8 (m, 1H), 9.67 (s, 1H), 8.2-8.4 (m, 1H), 8.1-8.2 (m, 1H), 7.5-7.6 (m, 2H), 7.2-7.4 (m, 1H), 6.6-6.9 (m, 2H), 4.2-4.4 (m, 2H), 3.84 (s, 3H), 3.5-3.7 (m, 2H), 3.12 (br s, 2H), 1.0-1.3 (m, 10H).

### Step D: 2-((2-(2-(-(3-Amino-6-methoxypyridin-2-yl)ethyl)(tert-butoxycarbonyl)amino)-methyl)-4,5-difluorophenyl)amino)-5-(trifluoromethyl)benzoic acid 47d

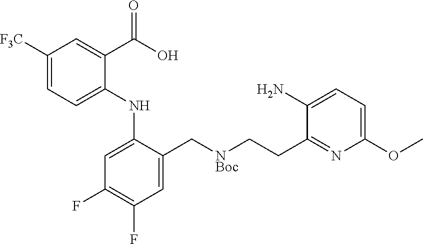 [SMILES: COc1ccc(N)c(CCN(Cc2cc(F)c(F)cc2Nc2ccc(C(F)(F)F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4,5-difluorophenyl)amino)-5-(trifluoromethyl)benzoic acid 47d (1.85 g, 79% yield) was prepared as a light brown foam. HPLC/MS 1.10 min (A), [M+H]⁺ 597.2. ¹H NMR (DMSO-d₆, 400 MHz): δ 10.4-11.7 (m, 1H), 8.17 (d, 1H, J=2.0 Hz), 7.3-7.5 (m, 2H), 7.0-7.2 (m, 1H), 6.94 (d, 1H, J=8.8 Hz), 6.6-6.8 (m, 1H), 6.32 (d, 1H, J=8.3 Hz), 4.5-5.0 (m, 1H), 4.32 (br s, 2H), 3.57 (s, 3H), 3.4-3.5 (m, 2H), 2.69 (br s, 2H), 1.2-1.5 (m, 11H).

### Step E: tert-Butyl 7.8-difluoro-15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 47e

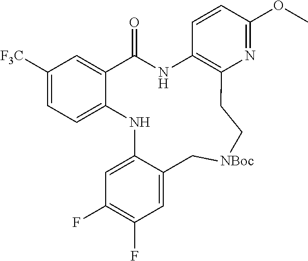 [SMILES: COc1ccc2c(n1)CCN(C(=O)OC(C)(C)C)Cc1cc(F)c(F)cc1Nc1ccc(C(F)(F)F)cc1C(=O)N2]

Following the procedure outlined in Example 41, Step E, and stirring the reaction mixture at room temperature for 18 h, tert-butyl 7,8-difluoro-15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 47e (1.32 g, 69% yield) was prepared. HPLC/MS 1.37 min (A), [M+H]⁺ 545.2. ¹H NMR (DMSO-d₆, 400 MHz): δ 9.99 (s, 1H), 7.8-8.0 (m, 3H), 7.7-7.8 (m, 1H), 7.41 (br s, 1H), 7.0-7.2 (m, 1H), 6.8-7.0 (m, 1H), 6.72 (d, 1H, J=8.8 Hz), 4.50 (s, 2H), 3.82 (s, 3H), 3.3-3.4 (m, 1H), 2.7-2.8 (m, 2H), 1.46 (br s, 9H).

### Step F: tert-Butyl 7,8-difluoro-15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate 47f

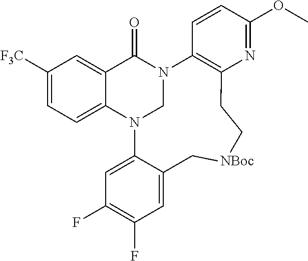 [SMILES: COc1ccc2c(n1)CCN(C(=O)OC(C)(C)C)Cc1cc(F)c(F)cc1N1CN2C(=O)c2cc(C(F)(F)F)ccc21]

Following the procedure outlined in Example 41, Step F, stirring the reaction at 90° C. for 43 h, tert-butyl 7,8-difluoro-15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate 47f (343 mg, 17% yield) was prepared. HPLC/MS 1.43 min (A), [M+H]⁺ 591.1.

### Step G: 7,8-Difluoro-2-(trifluoromethyl)-10,11,12,13-tetrahydro-19H-5,18-methano-dibenzo[c,f]pyrido[3,2-j][1,5,9]triazacyclotridecine-15,19(14H)-dione hydrochloride Example

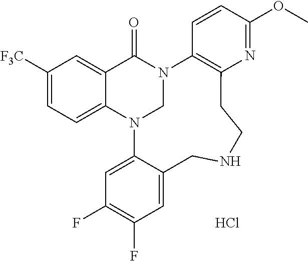 [SMILES: COc1ccc2c(n1)CCNCc1cc(F)c(F)cc1N1CN2C(=O)c2cc(C(F)(F)F)ccc21 ; Cl]

Following the procedure outlined in Example 41, Step G, stirring the reaction mixture at 90° C. for 20 h, 7,8-difluoro-2-(trifluoromethyl)-10,11,12,13-tetrahydro-19H-5,18-methano-dibenzo[c,]pyrido[3,2-j][1,5,9]triazacyclotridecine-15,19(14H)-dione hydrochloride (241.5 mg, 79% yield) was prepared. HPLC/MS 0.67 min (A), [M+H]⁺ 477.1. ¹H NMR (CD₃OD, 400 MHz): δ 8.32 (d, 1H, J=2.0 Hz), 7.81 (dd, 1H, J=8.8, 10.8 Hz), 7.6-7.8 (m, 3H), 6.4-6.8 (m, 2H), 5.61 (d, 1H, J=11.7 Hz), 4.98 (d, 1H, J=11.2 Hz), 4.3-4.4 (m, 1H), 4.1-4.3 (m, 1H), 3.4-3.5 (m, 1H), 3.2-3.3 (m, 2H), 3.12 (br d, 1H, J=4.9 Hz), 2.7-2.9 (m, 1H).

### Example 48

### 2,8,9-Trifluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

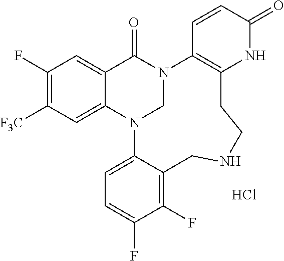 [SMILES: O=C1c2cc(F)c(C(F)(F)F)cc2N2CN1c1ccc(=O)[nH]c1CCNCc1c2ccc(F)c1F ; Cl]

### Step A: Methyl 2-((3,4-difluoro-2-formylphenyl)amino)-5-fluoro-4-(trifluoromethyl)-benzoate 48a

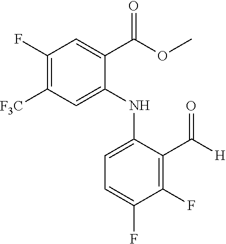 [SMILES: COC(=O)c1cc(F)c(C(F)(F)F)cc1Nc1ccc(F)c(F)c1C=O]

Following the preparation outlined in Example 7, Step A, substituting ethyl 2-bromo-5-(trifluoromethyl)benzoate with methyl 2-amino-5-fluoro-4-(trifluoromethyl)benzoate, 6-bromo-2,3-difluorobenzaldehyde with Int-3a, BINAP with Xantphos, toluene with 1,4-dioxane, and stirring the reaction at 80° C. overnight, methyl 2-((3,4-difluoro-2-formylphenyl)amino)-5-fluoro-4-(trifluoromethyl)benzoate 48a (2.91 g, 61% yield) was prepared as a yellow solid. HPLC/MS 1.35 min (B), [M+H]⁺ 378.0. ¹H NMR (CDCl₃, 400 MHz): δ 11.27 (s, 1H), 10.44 (s, 1H), 7.86 (d, J=10.27 Hz, 1H), 7.78 (d, J=5.87 Hz, 1H), 7.29-7.41 (m, 1H), 7.00-7.18 (m, 1H), 4.01 (s, 3H).

### Step B: Methyl 2-((2-(((tert-butoxycarbonyl)(2-(6-methoxy-3-nitropyridin-2-yl)ethyl-amino)methyl)-3,4-difluorophenyl)amino)-5-fluoro-4-(trifluoromethyl)benzoate 48b

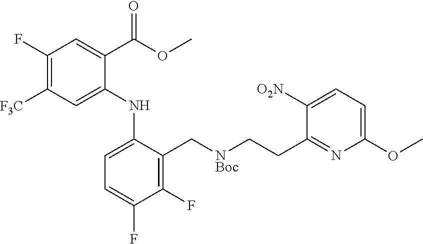 [SMILES: COC(=O)c1cc(F)c(C(F)(F)F)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3,4-difluoro-2-formylphenyl)amino)-5-fluoro-4-(trifluoromethyl)benzoate 48a, methyl 2-((2-(((tert-butoxy-carbonyl)(2-(6-methoxy-3-nitropyridin-2-yl)ethyl)amino)methyl)-3,4-difluorophenyl) amino)-5-fluoro-4-(trifluoromethyl)benzoate 48b (1.54 g, 89% yield) was prepared as a yellow oil, that solidified to a yellow solid. HPLC/MS 1.67 min (B), [M+H]⁺ 659.1. ¹H NMR (CDCl₃, 400 MHz): δ 9.15 (s, 1H), 8.19 (d, J=8.80 Hz, 1H), 7.72 (d, J=10.76 Hz, 1H), 7.13 (brd, J=9.29 Hz, 1H), 7.00-7.05 (m, 1H), 6.93 (br d, J=4.89 Hz, 1H), 6.65 (br d, J=8.80 Hz, 1H), 4.61 (s, 2H), 3.96 (s, 3H), 3.92 (s, 3H), 3.67 (br t, J=6.85 Hz, 2H), 3.28 (br t, J=6.85 Hz, 2H), 1.28 (s, 9H).

### Step C: 2-((2-(((tert-Butoxycarbonyl)(2-(6-methoxy-3-nitropyridin-2-yl)ethyl)amino)-methyl)-3,4-difluorophenyl)amino)-5-fluoro-4-(trifluoromethyl)benzoic acid 48c

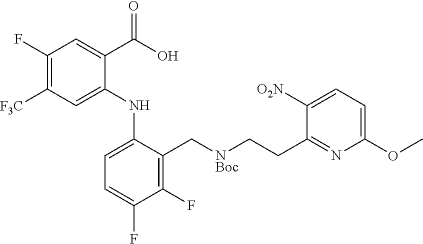 [SMILES: COc1ccc([N+](=O)[O-])c(CCN(Cc2c(Nc3cc(C(F)(F)F)c(F)cc3C(=O)O)ccc(F)c2F)C(=O)OC(C)(C)C)n1]

Following the procedure outlined in Example 41, Step C, stirring the reaction mixture at RT for 2 h, 2-((2-(((tert-butoxycarbonyl)(2-(6-methoxy-3-nitropyridin-2-yl)ethyl)amino) methyl)-3,4-difluorophenyl)amino)-5-fluoro-4-(trifluoromethyl)benzoic acid 48c (1.35 g, 88% yield) was prepared as a yellow powder. HPLC/MS 1.14 min (B), [M+H]⁺ 645.1. ¹H NMR (CDCl₃, 400 MHz): δ 9.20 (br s, 1H), 8.24 (d, J=9.29 Hz, 1H), 7.81 (d, J=10.76 Hz, 1H), 7.18 (q, J=9.29 Hz, 1H), 6.98-7.06 (m, 1H), 6.93 (br s, 1H), 6.69 (d, J=8.80 Hz, 1H), 4.62 (s, 2H), 3.96-4.05 (m, 3H), 3.71 (t, J=6.85 Hz, 2H), 3.32 (t, J=6.85 Hz, 2H), 1.26-1.32 (m, 9H).

### Step D: 2-((2-(((2-(3-Amino-6-methoxypyridin-2-yl)ethyl)(tert-butoxycarbonyl)amino)-methyl)-3,4-difluorophenyl)amino)-5-fluoro-4-(trifluoromethyl)benzoic acid 48d

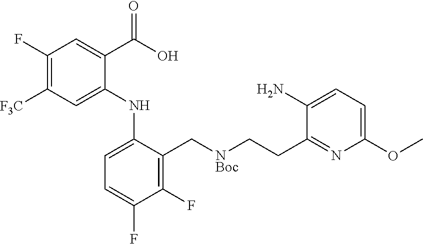 [SMILES: COc1ccc(N)c(CCN(Cc2c(Nc3cc(C(F)(F)F)c(F)cc3C(=O)O)ccc(F)c2F)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-3,4-difluorophenyl)amino)-5-fluoro-4-(trifluoromethyl)benzoic acid 48d (1.30 g, 98% yield) was prepared as a brown solid. HPLC/MS 1.05 min (B), [M+H]⁺ 615.2. ¹H NMR (CDCl₃, 400 MHz): δ 7.86 (br d, J=10.76 Hz, 1H), 7.05-7.21 (m, 2H), 6.91-7.03 (m, 2H), 6.49 (d, J=8.80 Hz, 1H), 4.57 (s, 2H), 3.83 (s, 3H), 3.36-3.47 (m, 2H), 2.97 (br s, 2H), 1.36-1.48 (m, 9H).

### Step E: tert-Butyl 2,8,9-trifluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 48e

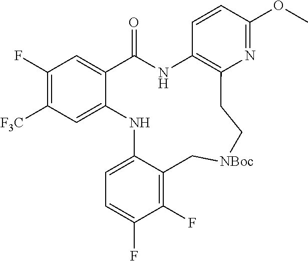 [SMILES: COc1ccc2c(n1)CCN(C(=O)OC(C)(C)C)Cc1c(ccc(F)c1F)Nc1cc(C(F)(F)F)c(F)cc1C(=O)N2]

Following the procedure outlined in Example 41, Step E, substituting HATU with Pyoxim and stirring the reaction mixture at room temperature for 1 h, tert-butyl 2,8,9-trifluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 48e (776 mg, 61% yield) was prepared. HPLC/MS 1.49 min (F), [M+H]⁺ 597.1. ¹H NMR (DMSO-d₆, 400 MHz): δ 10.17 (br s, 1H), 7.88 (br d, J=10.76 Hz, 1H), 7.72-7.84 (m, 2H), 7.54-7.68 (m, 1H), 7.16 (q, J=9.29 Hz, 1H), 6.72 (d, J=8.31 Hz, 1H), 6.14-6.29 (m, 1H), 4.79 (s, 2H), 3.83 (s, 3H), 3.17 (br s, 2H), 2.79 (br s, 2H), 1.46 (br s, 9H).

### Step F: tert-Butyl 2,8,9-trifluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 48f

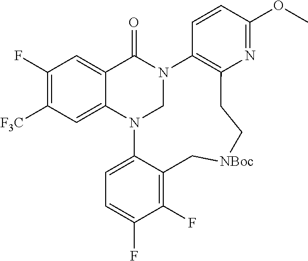 [SMILES: COc1ccc2c(n1)CCN(C(=O)OC(C)(C)C)Cc1c(ccc(F)c1F)N1CN2C(=O)c2cc(F)c(C(F)(F)F)cc21]

Following the procedure outlined in Example 41, Step F, stirring the reaction at 90° C. overnight and then RT for 4 h, tert-butyl 2,8,9-trifluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 48f (485 mg, 61% yield) was prepared. HPLC/MS 1.45 min (B), [M+H]⁺ 509.1. ¹H NMR (CDCl₃, 400 MHz): δ 7.81 (d, J=9.78 Hz, 1H), 7.33 (d, J=8.31 Hz, 1H), 7.12-7.18 (m, 1H), 6.84-6.90 (m, 1H), 6.54 (d, J=8.31 Hz, 1H), 6.36 (d, J=4.89 Hz, 1H), 5.43 (d, J=11.25 Hz, 1H), 5.03 (br d, J=14.18 Hz, 1H), 4.42 (d, J=11.25 Hz, 1H), 3.87 (br d, J=13.69 Hz, 2H), 3.78 (s, 3H), 2.92 (td, J=11.37, 2.69 Hz, 1H), 2.67-2.77 (m, 1H), 2.34-2.51 (m, 1H), 1.36 (s, 9H).

### Step G: 2,8,9-Trifluoro-3-(trifluoromethyl)-10,11,12,13-tetrahydro-19H-5,18-methano-dibenzo[c,f]pyrido[3,2-j][1,5,9]triazacyclotridecine-15,19(14H)-dione hydrochloride Example

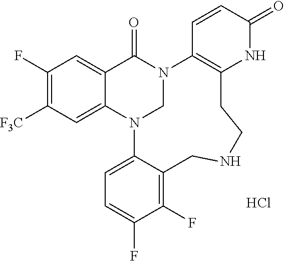 [SMILES: O=C1c2cc(F)c(C(F)(F)F)cc2N2CN1c1ccc(=O)[nH]c1CCNCc1c2ccc(F)c1F ; Cl]

Following the procedure outlined in Example 41, Step G, stirring the reaction mixture at 90° C. for 20 h, 2,8,9-trifluoro-3-(trifluoromethyl)-10,11,12,13-tetrahydro-19H-5,18-methano-dibenzo[c,]pyrido[3,2-j][1,5,9]triazacyclotridecine-15,19(14H)-dione hydrochloride (281.0 mg, 66% yield) was prepared. HPLC/MS 0.68 min (A), [M+H]⁺ 495.1. ¹H NMR (DMSO-d₆, 400 MHz): δ 10.48-10.67 (m, 1H), 9.13-9.35 (m, 1H), 7.98 (d, J=10.3 Hz, 1H), 7.77-7.93 (m, 1H), 7.47-7.68 (m, 2H), 6.57 (d, J=5.4 Hz, 1H), 6.43 (d, J=9.8 Hz, 1H), 5.51 (d, J=11.2 Hz, 1H), 5.15 (d, J=11.7 Hz, 1H), 4.27 (br s, 2H), 3.28-3.43 (m, 1H), 3.06-3.27 (m, 2H), 2.68-2.86 (m, 1H).

### Example 49

### 3-Chloro-2,8,9-trifluoro-10,11,12,13-tetrahydro-19H-5,18-methanodibenzo[c,f]pyrido[3,2-j][1,5,9]triazacyclotridecine-15,19(14H)-dione hydrochloride

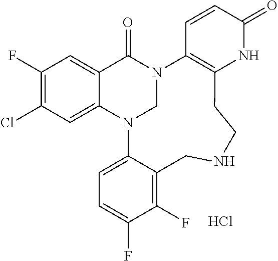 [SMILES: O=C1c2cc(F)c(Cl)cc2N2CN1c1ccc(=O)[nH]c1CCNCc1c2ccc(F)c1F ; Cl]

### Step A: Methyl 4-chloro-2-((3,4-difluoro-2-formylphenyl)amino)-5-fluorobenzoate 49a

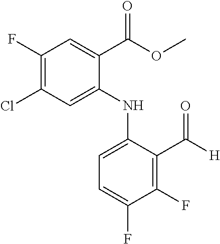 [SMILES: COC(=O)c1cc(F)c(Cl)cc1Nc1ccc(F)c(F)c1C=O]

Following the preparation outlined in Example 7, Step A, substituting ethyl 2-bromo-5-(trifluoromethyl)benzoate with methyl 2-amino-4-chloro-5-fluorobenzoate, 6-bromo-2,3-difluorobenzaldehyde with Int-3a, BINAP with Xantphos, toluene with 1,4-dioxane, and stirring the reaction at 100° C. for 20 h, methyl 4-chloro-2-((3,4-difluoro-2-formylphenyl)amino)-5-fluorobenzoate 49a (2.32 g, 30% yield) was prepared as a bright yellow solid. HPLC/MS 1.31 min (A), [M+H]⁺ 344.0. ¹H NMR (DMSO-d₆, 400 MHz): δ 10.89 (s, 1H), 10.29 (s, 1H), 7.88 (d, J=9.8 Hz, 1H), 7.72 (d, J=6.4 Hz, 1H), 7.67 (q, J=9.3 Hz, 1H), 7.24 (ddd, J=9.5, 3.4, 1.2 Hz, 1H), 3.88 (s, 3H).

### Step B: Methyl 2-((2-(((tert-butoxycarbonyl)(2-(6-methoxy-3-nitropyridin-2-yl)ethyl-amino)methyl)-3,4-difluorophenyl)amino)-4-chloro-5-fluorobenzoate 49b

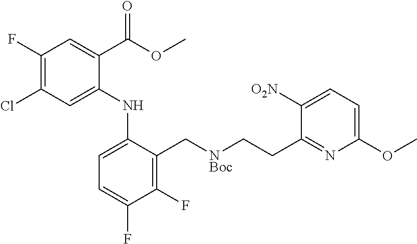 [SMILES: COC(=O)c1cc(F)c(Cl)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4-chloro-2-((3,4-difluoro-2-formylphenyl)amino)-5-fluorobenzoate 49a, methyl 2-((2-(((tert-butoxycarbonyl)(2-(6-methoxy-3-nitropyridin-2-yl)ethyl)amino)methyl)-3,4-difluorophenyl)amino)-4-chloro-5-fluorobenzoate 49b (2.20 g, 55% yield) was prepared. HPLC/MS 1.64 min (A), [M+H]⁺ 625.2. ¹H NMR (DMSO-d₆, 400 MHz): δ 8.96 (s, 1H), 8.33 (br d, J=8.8 Hz, 1H), 7.74 (d, J=9.8 Hz, 1H), 7.40-7.49 (m, 1H), 7.14-7.21 (m, 1H), 6.87 (br d, J=7.8 Hz, 1H), 6.68 (br d, J=4.9 Hz, 1H), 4.50 (s, 2H), 3.91 (br s, 3H), 3.86 (s, 3H), 3.55 (br s, 2H), 3.14 (t, J=6.6 Hz, 2H), 1.14 (s, 9H).

### Step C: 2-((2-(((tert-Butoxycarbonyl)(2-(6-methoxy-3-nitropyridin-2-yl)ethyl)amino)-methyl)-3,4-difluorophenyl)amino)-4-chloro-5-fluorobenzoic acid 49c

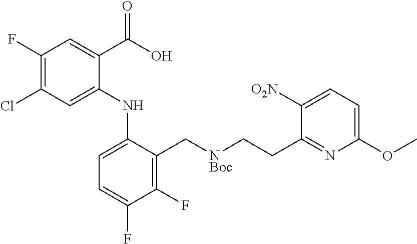 [SMILES: COc1ccc([N+](=O)[O-])c(CCN(Cc2c(Nc3cc(Cl)c(F)cc3C(=O)O)ccc(F)c2F)C(=O)OC(C)(C)C)n1]

Following the procedure outlined in Example 41, Step C, stirring the reaction mixture at RT for 16 h, 2-((2-(((tert-butoxycarbonyl)(2-(6-methoxy-3-nitropyridin-2-yl)ethyl)amino)-methyl)-3,4-difluorophenyl)amino)-4-chloro-5-fluorobenzoic acid 49c (2.22 g, 97% yield) was prepared as a foamy solid. HPLC/MS 1.53 min (A), [M+H]⁺ 611.1. ¹H NMR (DMSO-d₆, 400 MHz): δ 13.34-13.70 (m, 1H), 9.28 (s, 1H), 8.33 (br d, J=9.3 Hz, 1H), 7.72 (d, J=9.8 Hz, 1H), 7.39-7.49 (m, 1H), 7.18 (td, J=4.5, 3.2 Hz, 1H), 6.87 (br d, J=5.4 Hz, 1H), 6.64 (br s, 1H), 4.50 (br s, 2H), 3.91 (br s, 3H), 3.57 (br t, J=6.4 Hz, 2H), 3.14 (br t, J=6.4 Hz, 2H), 1.12 (br s, 9H).

### Step D: 2-((2-(((2-(3-Amino-6-methoxypyridin-2-yl)ethyl)(tert-butoxycarbonyl)amino)-methyl)-3,4-difluorophenyl)amino)-4-chloro-5-fluorobenzoic acid 49d

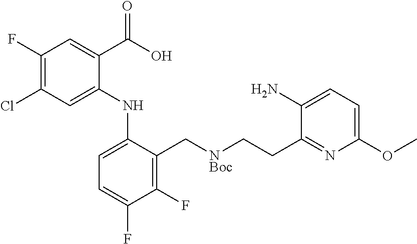 [SMILES: COc1ccc(N)c(CCN(Cc2c(Nc3cc(Cl)c(F)cc3C(=O)O)ccc(F)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3,4-difluorophenyl)amino)-4-chloro-5-fluorobenzoic acid 49d (2.02 g, 104% yield) was prepared as a solid. HPLC/MS 1.12 min (A), [M+H]⁺ 581.1. ¹H NMR (DMSO-d₆, 400 MHz): δ 7.69 (d, J=10.3 Hz, 1H), 7.27-7.37 (m, 1H), 7.03-7.10 (m, 1H), 6.94 (d, J=8.3 Hz, 1H), 6.55 (br d, J=6.4 Hz, 1H), 6.32 (d, J=8.8 Hz, 1H), 4.50 (br s, 2H), 3.61 (s, 3H), 3.34-3.40 (m, 2H), 2.62 (br t, J=7.1 Hz, 2H), 1.22 (s, 9H).

### Step E: tert-Butyl 3-chloro-2,8,9-trifluoro-15-methoxy-19-oxo-5,10,12,13,18,19-hexa-hydro-11H-dibenzo[c,f]pyrido[3,2-j][1,5,9]triazacyclotridecine-11-carboxylate 49e

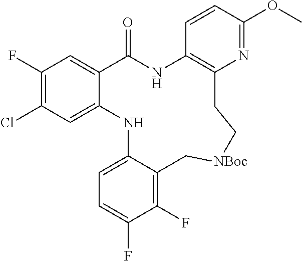 [SMILES: COc1ccc2c(n1)CCN(C(=O)OC(C)(C)C)Cc1c(ccc(F)c1F)Nc1cc(Cl)c(F)cc1C(=O)N2]

Following the procedure outlined in Example 41, Step E, substituting HATU with pyoxim and stirring the reaction mixture at RT for 23 h, tert-butyl 3-chloro-2,8,9-trifluoro-15-methoxy-19-oxo-5,10,12,13,18,19-hexahydro-11H-dibenzo[c,f]pyrido[3,2-j][1,5,9]triazacyclotridecine-11-carboxylate 49e (502 mg, 26% yield) was prepared as a foamy solid. HPLC/MS 1.43 min (A), [M+H]⁺ 563.1. ¹H NMR (DMSO-d₆, 400 MHz): δ 10.21 (br s, 1H), 7.87 (br d, J=8.3 Hz, 1H), 7.82 (br d, J=9.3 Hz, 1H), 7.72 (s, 1H), 7.48 (br d, J=2.4 Hz, 1H), 7.16 (q, J=9.3 Hz, 1H), 6.70 (d, J=8.8 Hz, 1H), 6.18 (br d, J=6.8 Hz, 1H), 4.78 (s, 2H), 3.81 (s, 3H), 3.18 (br s, 2H), 2.74 (br s, 2H), 1.42 (br s, 9H).

### Step F: tert-Butyl 3-chloro-2,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 49f

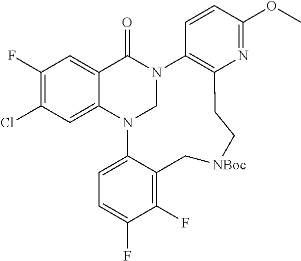 [SMILES: COc1ccc2c(n1)CCN(C(=O)OC(C)(C)C)Cc1c(ccc(F)c1F)N1CN2C(=O)c2cc(F)c(Cl)cc21]

Following the procedure outlined in Example 41, Step F, stirring the reaction at 85° C. for 22 h, tert-butyl 3-chloro-2,8,9-trifluoro-15-methoxy-19-oxo-12,13-dihydro-19H-5,18-methano-dibenzo[c,f]pyrido[3,2-j][1,5,9]triazacyclotridecine-11(10H)-carboxylate 49f (324 mg, 63% yield) was prepared. HPLC/MS 1.40 min (A), [M+H]⁺ 575.2. ¹H NMR (DMSO-d₆, 400 MHz): δ 7.79 (d, J=9.3 Hz, 1H), 7.70 (d, J=8.8 Hz, 1H), 7.60 (q, J=9.3 Hz, 1H), 7.32 (br dd, J=8.1, 4.2 Hz, 1H), 6.79 (d, J=8.8 Hz, 1H), 6.53 (d, J=5.9 Hz, 1H), 5.59 (d, J=11.7 Hz, 1H), 5.02 (br d, J=14.2 Hz, 1H), 4.80 (br d, J=10.3 Hz, 1H), 3.91 (br s, 1H), 3.86 (s, 3H), 3.49-3.80 (m, 2H), 2.94 (br t, J=9.8 Hz, 2H), 1.41 (s, 9H).

### Step G: 3-Chloro-2,8,9-trifluoro-10,11,12,13-tetrahydro-19H-5,18-methanodibenzo-[c,f]pyrido[3,2-j][1,5,9]triazacyclotridecine-15,19(14H)-dione hydrochloride Example 49

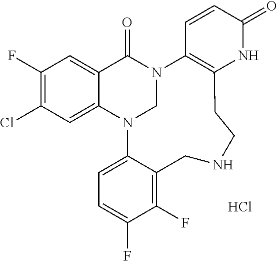 [SMILES: O=C1c2cc(F)c(Cl)cc2N2CN1c1ccc(=O)[nH]c1CCNCc1c2ccc(F)c1F ; Cl]

Following the procedure outlined in Example 41, Step G, stirring the reaction mixture at 90° C. for 4 h, 3-chloro-2,8,9-trifluoro-10,11,12,13-tetrahydro-19H-5,18-methanodibenzo-[c,f]pyrido[3,2-j][1,5,9]triazacyclotridecine-15,19(14H)-dione hydrochloride (234 mg, 94% yield) was prepared. HPLC/MS 0.62 min (A), [M+H]⁺ 461.1. ¹H NMR (DMSO-d₆, 400 MHz): δ 10.41 (br s, 1H), 9.12 (br s, 1H), 7.73-7.83 (m, 2H), 7.46 (d, J=9.78 Hz, 2H), 6.45 (d, J=5.87 Hz, 1H), 6.34 (d, J=9.29 Hz, 1H), 5.43 (d, J=11.25 Hz, 1H), 5.01 (d, J=11.25 Hz, 1H), 4.19-4.27 (m, 1H), 4.07-4.16 (m, 1H), 3.22-3.33 (m, 1H), 2.99-3.10 (m, 2H), 2.65-2.75 (m, 1H).

### Example 50

### Step A: 3-Chloro-2,8,9-trifluoro-10,11,12,13-tetrahydro-19H-5,18-methanodibenzo-[c,f]pyrido[3,2-j][1,5,9]triazacyclotridecine-15,19(14H)-dione

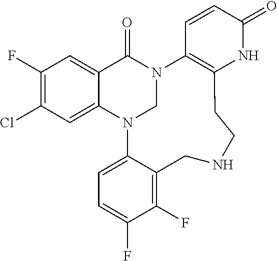 [SMILES: O=C1c2cc(F)c(Cl)cc2N2CN1c1ccc(=O)[nH]c1CCNCc1c2ccc(F)c1F]

3-Chloro-2,8,9-trifluoro-10,11,12,13-tetrahydro-19H-5,18-methanodibenzo[c,f]pyrido [3,2-j][1,5,9]triazacyclotridecine-15,19(14H)-dione hydrochloride Example 49 (234 mg, 0.479 mmol) was purified over a Waters XSelect 5 μm C18, 150×30 mm column, eluting with 30-99% AcCN in 10 mM ammonium bicarbonate in H₂O (adjusted to pH 10 with ammonia) to afford 3-chloro-2,8,9-trifluoro-10,11,12,13-tetrahydro-19H-5,18-methanodibenzo[c,f]pyrido[3,2-j][1,5,9]-triazacyclotridecine-15,19(14H)-dione (57 mg, 26% yield). HPLC/MS 0.65 min (A), [M+H]⁺ 461.1. ¹H NMR (DMSO-d₆, 400 MHz): δ 11.85 (br s, 1H), 7.76 (d, J=9.29 Hz, 1H), 7.49-7.58 (m, 1H) 7.37-7.46 (m, 2H), 6.57 (d, J=6.36 Hz, 1H), 6.21 (br d, J=9.29 Hz, 1H), 5.35 (d, J=9.78 Hz, 1H), 5.02 (d, J=9.29 Hz, 1H), 3.64-3.74 (m, 2H), 2.80-3.01 (m, 3H), 2.20-2.28 (m, 1H).

### Example 51

### 2-Fluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

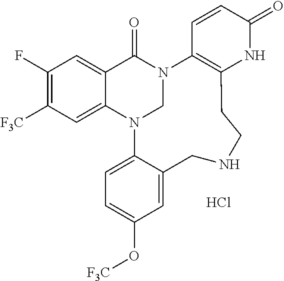 [SMILES: O=C1c2cc(F)c(C(F)(F)F)cc2N2CN1c1ccc(=O)[nH]c1CCNCc1cc(OC(F)(F)F)ccc12 ; Cl]

### Step A: Methyl 5-fluoro-2-((2-formyl-4-(trifluoromethoxy)phenyl)amino)-4-(trifluoro-methyl)benzoate 51a

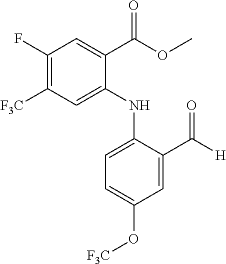 [SMILES: COC(=O)c1cc(F)c(C(F)(F)F)cc1Nc1ccc(OC(F)(F)F)cc1C=O]

Following the preparation outlined in Example 7, Step A, substituting ethyl 2-bromo-5-(trifluoromethyl)benzoate with methyl 2-amino-5-fluoro-4-(trifluoromethyl)benzoate, 2-bromo-5-(trifluoromethoxy)benzaldehyde with Int-3a, BINAP with Xantphos, toluene with 1,4-dioxane, and stirring the reaction at 80° C. overnight, methyl 5-fluoro-2-((2-formyl-4-(trifluoromethoxy)phenyl)amino)-4-(trifluoromethyl)benzoate 51a (718 mg, 40% yield) was prepared as a yellow solid. HPLC/MS 1.44 min (B), [M+H]⁺ 415.1. ¹H NMR (CDCl₃, 400 MHz): δ 11.24 (s, 1H), 9.99 (s, 1H), 7.87 (d, J=10.3 Hz, 1H), 7.82 (d, J=5.9 Hz, 1H), 7.57 (d, J=2.4 Hz, 1H), 7.40-7.44 (m, 1H), 7.38 (br d, J=2.9 Hz, 1H), 4.01 (s, 3H).

### Step B: Methyl 2-((2-(((tert-butoxycarbonyl)(2-(6-methoxy-3-nitropyridin-2-yl)ethyl-amino)methyl)-4-(trifluoromethoxy)phenyl)amino)-5-fluoro-4(trifluoromethyl) benzoate 51b

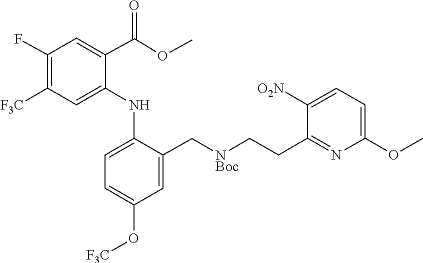 [SMILES: COC(=O)c1cc(F)c(C(F)(F)F)cc1Nc1ccc(OC(F)(F)F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-fluoro-2-((2-formyl-4-(trifluoromethoxy)phenyl)amino)-4-(trifluoromethyl)benzoate 51a, methyl 2-((2-(((tert-butoxycarbonyl)(2-(6-methoxy-3-nitropyridin-2-yl)ethyl)amino)methyl)-4-(trifluoromethoxy) phenyl)amino)-5-fluoro-4-(trifluoromethyl)benzoate 51b (2.20 g, 55% yield) was prepared as a clear oil. HPLC/MS 1.73 min (B), [M+H]⁺ 707.1. ¹H NMR (CDCl₃, 400 MHz): δ 9.16 (s, 1H), 8.19 (d, J=8.8 Hz, 1H), 7.77 (d, J=10.8 Hz, 1H), 7.28-7.32 (m, 1H), 7.23 (br s, 1H), 7.17 (br d, J=8.3 Hz, 1H), 6.99 (br s, 1H), 6.64 (br d, J=8.8 Hz, 1H), 4.49 (br s, 2H), 3.93-3.97 (m, 6H), 3.70 (br s, 2H), 3.30 (br s, 2H), 1.37 (s, 9H).

### Step C: 2-((2-(((tert-Butoxycarbonyl)(2-(6-methoxy-3-nitropyridin-2-yl)ethyl)amino)-methyl)-4-(trifluoromethoxy)phenyl)amino)-5-fluoro-4-(trifluoromethyl)benzoic acid 51c

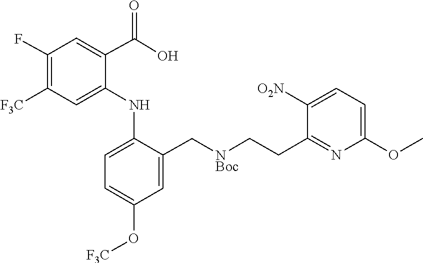 [SMILES: COc1ccc([N+](=O)[O-])c(CCN(Cc2cc(OC(F)(F)F)ccc2Nc2cc(C(F)(F)F)c(F)cc2C(=O)O)C(=O)OC(C)(C)C)n1]

Following the procedure outlined in Example 41, Step C, stirring the reaction mixture at RT for 16 h, 2-((2-(((tert-butoxycarbonyl)(2-(6-methoxy-3-nitropyridin-2-yl)ethyl)amino)-methyl)-4-(trifluoromethoxy)phenyl)amino)-5-fluoro-4-(trifluoromethyl)benzoic acid 51c (1.39 g, 100% yield) was prepared as a yellow powder. HPLC/MS 1.19 min (B), [M+H]⁺ 693.1. ¹H NMR (CDCl₃, 400 MHz): δ 9.27 (br s, 1H), 8.22 (d, J=8.8 Hz, 1H), 7.64 (br d, J=5.9 Hz, 1H), 7.14-7.27 (m, 1H), 7.01-7.14 (m, 2H), 6.93-7.01 (m, 1H), 6.68 (d, J=9.3 Hz, 1H), 4.66 (br s, 2H), 3.95 (s, 3H), 3.79 (br t, J=7.1 Hz, 2H), 3.36 (t, J=6.8 Hz, 2H), 1.39-1.53 (m, 9H).

### Step D: 2-((2-(((2-(3-Amino-6-methoxypyridin-2-yl)ethyl)(tert-butoxycarbonyl)amino) methyl)-4-(trifluoromethoxy)phenyl)amino)-5-fluoro-4-(trifluoromethyl)benzoic acid 51d

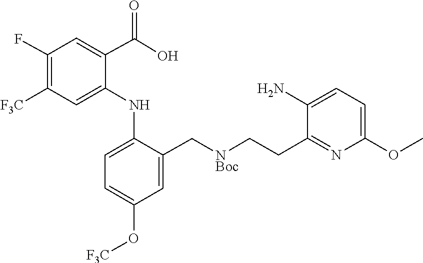 [SMILES: COc1ccc(N)c(CCN(Cc2cc(OC(F)(F)F)ccc2Nc2cc(C(F)(F)F)c(F)cc2C(=O)O)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-4-(trifluoromethoxy)phenyl) amino)-5-fluoro-4-(trifluoromethyl)benzoic acid 51d (1.22 g, 100% yield) was prepared as a gray solid. HPLC/MS 1.12 min (B), [M+H]⁺ 663.1. ¹H NMR (CDCl₃, 400 MHz): δ 7.71-7.88 (m, 1H) 7.17-7.28 (m, 2H) 7.07-7.16 (m, 2H) 7.04 (br d, J=5.4 Hz, 1H) 6.51 (br d, J=8.8 Hz, 1H) 4.48 (br s, 2H) 3.81 (s, 3H) 3.39-3.66 (m, 2H) 2.84-3.04 (m, 2H) 1.49 (s, 9H).

### Step E: tert-Butyl 2-fluoro-15-methoxy-19-oxo-8-(trifluoromethoxy)-3-(trifluoro-methyl)-5,10,12,13,18,19-hexahydro-11H-dibenzo[c,f]pyrido[3,2-j][1,5,9]triazacyclo-tridecine-11-carboxylate 51e

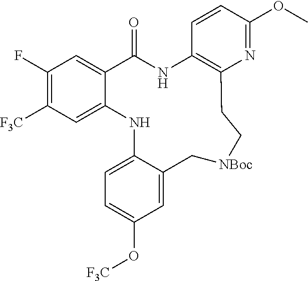 [SMILES: COc1ccc2c(n1)CCN(C(=O)OC(C)(C)C)Cc1cc(OC(F)(F)F)ccc1Nc1cc(C(F)(F)F)c(F)cc1C(=O)N2]

Following the procedure outlined in Example 41, Step E, substituting HATU with pyoxim and stirring the reaction mixture at RT over the weekend, tert-butyl 2-fluoro-15-methoxy-19-oxo-8-(trifluoromethoxy)-3-(trifluoromethyl)-5,10,12,13,18,19-hexahydro-11H-dibenzo[c,f]pyrido-[3,2-j][1,5,9]triazacyclotridecine-11-carboxylate 51e (577 mg, 51% yield) was prepared as a foamy solid. HPLC/MS 1.54 min (B), [M+H]⁺ 645.2. ¹H NMR (CDCl₃, 400 MHz): δ 10.81 (br s, 1H), 8.19 (d, J=10.8 Hz, 1H), 8.11 (d, J=8.8 Hz, 1H), 7.45 (d, J=6.4 Hz, 1H), 7.06 (d, J=2.4 Hz, 1H), 6.94 (dd, J=8.8, 2.0 Hz, 1H), 6.50 (d, J=8.8 Hz, 1H), 6.42 (br s, 1H), 6.13 (d, J=8.8 Hz, 1H), 3.73 (s, 3H), 2.11 (br s, 2H), 1.47 (s, 2H), 1.14 (br s, 2H), 0.92 (s, 9H).

### Step F: tert-Butyl 2-fluoro-15-methoxy-19-oxo-8-(trifluoromethoxy)-3-(trifluoromethyl)-12,13-dihydro-19H-5,18-methanodibenzo[c,f]pyrido[3,2-j][1,5,9]triazacyclotridecine-11(10H)-carboxylate 51f

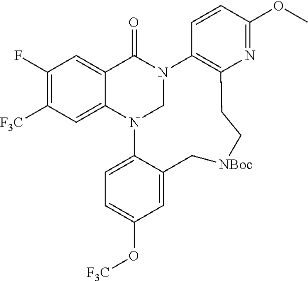 [SMILES: COc1ccc2c(n1)CCN(C(=O)OC(C)(C)C)Cc1cc(OC(F)(F)F)ccc1N1CN2C(=O)c2cc(F)c(C(F)(F)F)cc21]

Following the procedure outlined in Example 41, Step F, stirring the reaction at 85° C. for 22 h, tert-butyl 2-fluoro-15-methoxy-19-oxo-8-(trifluoromethoxy)-3-(trifluoromethyl)-12,13-dihydro-19H-5,18-methanodibenzo[c,f]pyrido[3,2-j][1,5,9]triazacyclotridecine-11(10H)-carboxylate 51f (148 mg, 25% yield) was prepared as a tan solid. HPLC/MS 1.56 min (B), [M+H]⁺ 657.2. ¹H NMR (CDCl₃, 400 MHz): δ 7.81 (d, J=10.3 Hz, 1H) 7.45-7.48 (m, 1H) 7.35 (d, J=8.8 Hz, 1H) 7.10-7.19 (m, 2H) 6.54 (d, J=8.3 Hz, 1H) 6.39 (d, J=5.4 Hz, 1H) 5.47 (d, J=11.2 Hz, 1H) 4.72 (br d, J=14.2 Hz, 1H) 4.48 (d, J=11.2 Hz, 1H) 3.90 (br d, J=14.7 Hz, 1H) 3.74-3.83 (m, 4H) 2.93 (td, J=11.7, 2.9 Hz, 1H) 2.69 (td, J=12.0, 6.8 Hz, 1H) 2.44 (br s, 1H), 1.41 (s, 9).

### Step G: 2-Fluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 51

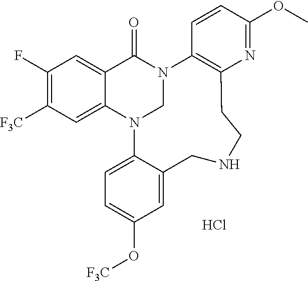 [SMILES: COc1ccc2c(n1)CCNCc1cc(OC(F)(F)F)ccc1N1CN2C(=O)c2cc(F)c(C(F)(F)F)cc21 ; Cl]

Following the procedure outlined in Example 41, Step G, stirring the reaction mixture at 90° C. for 20 h, 2-fluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (100 mg, 74% yield) was prepared as a white solid. HPLC/MS 1.16 min (B), [M+H]⁺ 543.1. ¹H NMR (DMSO-d₆, 400 MHz): δ 7.88-7.99 (m, 2H), 7.72-7.77 (m, 1H), 7.61-7.67 (m, 1H), 7.46 (d, J=9.3 Hz, 1H), 6.46 (d, J=5.4 Hz, 1H), 6.35 (d, J=9.8 Hz, 1H), 5.48 (d, J=11.2 Hz, 1H), 5.00 (d, J=11.7 Hz, 1H), 4.17-4.26 (m, 1H), 4.09 (br d, J=5.4 Hz, 1H), 3.16-3.24 (m, 1H), 3.07-3.14 (m, 1H), 2.97-3.06 (m, 1H), 2.62-2.70 (m, 1H).

### Example 52

### 2,9-Difluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

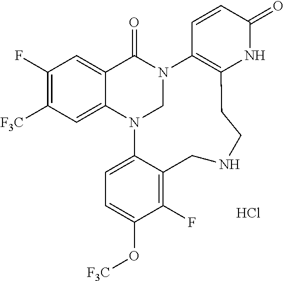 [SMILES: O=C1c2cc(F)c(C(F)(F)F)cc2N2CN1c1ccc(=O)[nH]c1CCNCc1c2ccc(OC(F)(F)F)c1F ; Cl]

### Step A: Methyl 5-fluoro-2-((3-fluoro-2-formyl-4-(trifluoromethoxy)phenyl)amino)-4-(trifluoromethyl)benzoate 52a

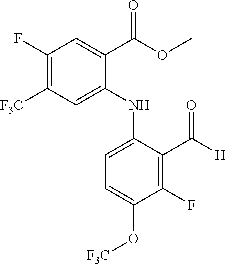 [SMILES: COC(=O)c1cc(F)c(C(F)(F)F)cc1Nc1ccc(OC(F)(F)F)c(F)c1C=O]

Following the preparation outlined in Example 7, Step A, substituting ethyl 2-bromo-5-(trifluoromethyl)benzoate with methyl 2-amino-5-fluoro-4-(trifluoromethyl)benzoate, 6-bromo-2-fluoro-3-(trifluoromethoxy)benzaldehyde with Int-3a, BINAP with Xantphos, toluene with 1,4-dioxane, and stirring the reaction at 80° C. overnight, methyl 5-fluoro-2-((3-fluoro-2-formyl-4-(trifluoromethoxy)phenyl)amino)-4-(trifluoromethyl)benzoate 52a (937 mg, 49% yield) was prepared as a yellow solid. HPLC/MS 1.44 min (B), [M+H]⁺ 444.0. ¹H NMR (CDCl₃, 400 MHz): δ 11.45 (s, 1H), 10.44 (s, 1H), 7.88 (d, J=10.3 Hz, 1H), 7.82 (d, J=6.4 Hz, 1H), 7.40 (t, J=8.9 Hz, 1H), 7.08 (dd, J=9.3, 1.0 Hz, 1H) 4.01 (s, 3H).

### Step B: Methyl 2-((2-(((tert-butoxycarbonyl)(2-(6-methoxy-3-nitropyridin-2-yl)ethyl-amino)methyl)-3-fluoro-4-(trifluoromethoxy)phenylamino)-5-fluoro-4-(trifluoromethyl)-benzoate 52b

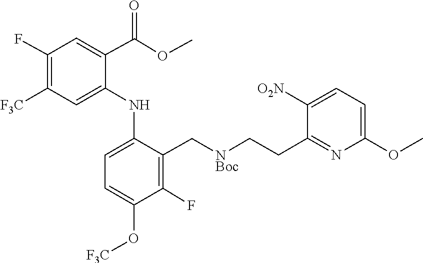 [SMILES: COC(=O)c1cc(F)c(C(F)(F)F)cc1Nc1ccc(OC(F)(F)F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-fluoro-2-((3-fluoro-2-formyl-4-(trifluoromethoxy)phenyl)amino)-4-(trifluoromethyl)benzoate 52a, methyl 2-((2-(((tert-butoxycarbonyl)(2-(6-methoxy-3-nitropyridin-2-yl)ethyl)amino)methyl)-3-fluoro-4-(trifluoromethoxy)phenyl)amino)-5-fluoro-4-(trifluoromethyl)benzoate 52b (1.35 g, 84% yield) was prepared. HPLC/MS 1.71 min (B), [M+H]⁺ 725.1. ¹H NMR (CDCl₃, 400 MHz): δ 9.39 (s, 1H), 8.41 (d, J=9.3 Hz, 1H), 7.95 (d, J=10.8 Hz, 1H), 7.43 (s, 1H), 7.37 (br d, J=5.9 Hz, 1H), 7.25 (br d, J=8.8 Hz, 1H), 6.86 (d, J=8.8 Hz, 1H), 4.83-4.87 (m, 2H), 4.18 (s, 3H), 4.11 (s, 3H), 3.89 (t, J=6.8 Hz, 2H), 3.50 (t, J=6.8 Hz, 2H), 1.49 (s, 9H)

### Step C: 2-((2-(((tert-Butoxycarbonyl)(2-(6-methoxy-3-nitropyridin-2-yl)ethyl)amino)-methyl)-3-fluoro-4-(trifluoromethoxy)phenyl)amino)-5-fluoro-4-(trifluoromethyl)benzoic acid 52c

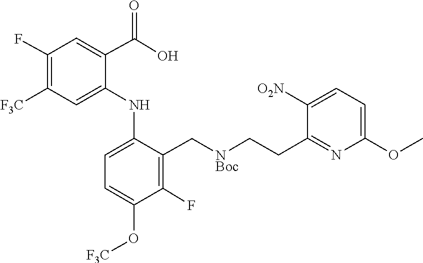 [SMILES: COc1ccc([N+](=O)[O-])c(CCN(Cc2c(Nc3cc(C(F)(F)F)c(F)cc3C(=O)O)ccc(OC(F)(F)F)c2F)C(=O)OC(C)(C)C)n1]

Following the procedure outlined in Example 41, Step C, stirring the reaction mixture at ambient temperature for 16 h, 2-((2-(((tert-butoxycarbonyl)(2-(6-methoxy-3-nitropyridin-2-yl)ethyl)amino)methyl)-3-fluoro-4-(trifluoromethoxy)phenyl)amino)-5-fluoro-4-(trifluoro-methyl)benzoic acid 52c (1.39 g, 100% yield) was prepared as a yellow powder. HPLC/MS 1.22 min (B), [M+H]⁺ 711.1. ¹H NMR (CDCl₃, 400 MHz): δ 9.29 (br s, 1H), 8.25 (d, J=8.8 Hz, 1H), 7.83 (d, J=10.8 Hz, 1H), 7.23-7.30 (m, 2H), 7.15 (br s, 1H), 7.08 (br d, J=8.8 Hz, 1H), 6.70 (d, J=9.3 Hz, 1H), 4.66 (s, 2H), 4.01 (s, 3H), 3.73 (t, J=6.8 Hz, 2H), 3.34 (t, J=6.8 Hz, 2H), 1.27-1.34 (m, 9H).

### Step D: 2-((2-(((2-(3-Amino-6-methoxypyridin-2-yl)ethyl)(tert-butoxycarbonyl)amino)-methyl)-3-fluoro-4-(trifluoromethoxy)phenyl)amino)-5-fluoro-4-(trifluoromethyl)benzoic acid 52d

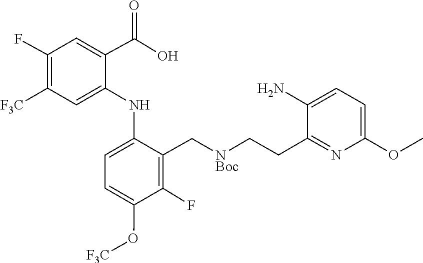 [SMILES: COc1ccc(N)c(CCN(Cc2c(Nc3cc(C(F)(F)F)c(F)cc3C(=O)O)ccc(OC(F)(F)F)c2F)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-3-fluoro-4-(trifluoromethoxy) phenyl)amino)-5-fluoro-4-(trifluoromethyl)benzoic acid 52d (1.05 g, 90% yield) was prepared as a gray solid. HPLC/MS 1.11 min (B), [M+H]⁺ 681.2. ¹H NMR (CDCl₃, 400 MHz): δ 7.90 (br d, J=11.2 Hz, 1H), 7.13-7.27 (m, 3H), 7.05 (br d, J=8.8 Hz, 1H), 6.50 (br d, J=8.8 Hz, 1H), 4.63 (s, 2H), 3.82 (s, 3H), 3.42 (br t, J=6.4 Hz, 2H), 2.94-3.05 (m, 2H), 1.44 (s, 9H).

### Step E: tert-Butyl 2,9-difluoro-15-methoxy-19-oxo-8-(trifluoromethoxy)-3-(trifluoro-methyl)-5,10,12,13,18,19-hexahydro-11H-dibenzo[c,f]pyrido[3,2-j][1,5,9]triazacyclo-tridecine-11-carboxylate 52e

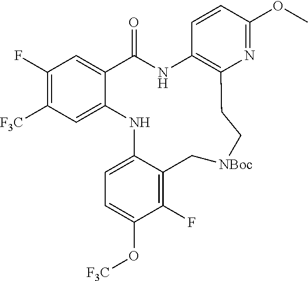 [SMILES: COc1ccc2c(n1)CCN(C(=O)OC(C)(C)C)Cc1c(ccc(OC(F)(F)F)c1F)Nc1cc(C(F)(F)F)c(F)cc1C(=O)N2]

Following the procedure outlined in Example 41, Step E, substituting HATU with pyoxim and stirring the reaction mixture at room temperature over the weekend, tert-butyl 2,9-difluoro-15-methoxy-19-oxo-8-(trifluoromethoxy)-3-(trifluoromethyl)-5,10,12,13,18,19-hexahydro-11H-dibenzo[c,]pyrido[3,2-j][1,5,9]triazacyclotridecine-11-carboxylate 52e (502 mg, 49% yield) was prepared. HPLC/MS 1.58 min (B), [M+H]⁺ 663.2. ¹H NMR (CDCl₃, 400 MHz): δ 10.48 (br s, 1H), 8.19 (d, J=10.8 Hz, 1H), 8.10 (d, J=8.8 Hz, 1H), 7.46 (d, J=5.9 Hz, 1H), 7.14 (s, 1H), 6.98 (t, J=8.7 Hz, 1H), 6.64 (br s, 1H), 6.51 (d, J=8.8 Hz, 1H), 5.86-5.98 (m, 1H), 4.74-5.11 (m, 1H), 3.74 (s, 3H), 1.96-2.41 (m, 2H), 1.51 (br s, 2H), 0.71-1.01 (m, 9H).

### Step F: tert-Butyl 2,9-difluoro-15-methoxy-19-oxo-8-(trifluoromethoxy)-3-(trifluoro-methyl)-12,13-dihydro-19H-5,18-methanodibenzo[c,f]pyrido[3,2-j][1,5,9]triazacyclo-tridecine-11(10H)-carboxylate 52f

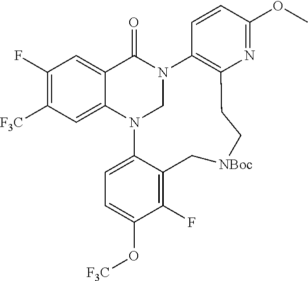 [SMILES: COc1ccc2c(n1)CCN(C(=O)OC(C)(C)C)Cc1c(ccc(OC(F)(F)F)c1F)N1CN2C(=O)c2cc(F)c(C(F)(F)F)cc21]

Following the procedure outlined in Example 41, Step F, stirring the reaction at 90° C. for the weekend, tert-butyl 2,9-difluoro-15-methoxy-19-oxo-8-(trifluoromethoxy)-3-(trifluoromethyl)-12,13-dihydro-19H-5,18-methanodibenzo[c,]pyrido[3,2-j][1,5,9]triazacyclo-tridecine-11(10H)-carboxylate 52f (339 mg, 65% yield) was prepared as a light tan solid. HPLC/MS 1.53 min (B), [M+H]⁺ 675.1. ¹H NMR (CDCl₃, 400 MHz): δ 7.82 (d, J=9.8 Hz, 1H), 7.22-7.39 (m, 2H), 6.92 (dd, J=8.8, 2.0 Hz, 1H), 6.53 (d, J=8.8 Hz, 1H), 6.37 (d, J=5.4 Hz, 1H), 5.45 (d, J=11.2 Hz, 1H), 5.03 (br d, J=13.7 Hz, 1H), 4.45 (d, J=11.2 Hz, 1H), 3.61-4.02 (m, 5H), 2.80-2.96 (m, 1H), 2.73 (br d, J=7.3 Hz, 1H), 2.30-2.48 (m, 1H), 1.44 (s, 9H).

### Step G: 2,9-Difluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

### Example 52

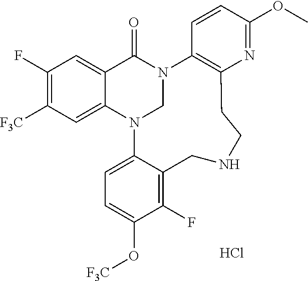 [SMILES: COc1ccc2c(n1)CCNCc1c(ccc(OC(F)(F)F)c1F)N1CN2C(=O)c2cc(F)c(C(F)(F)F)cc21 ; Cl]

Following the procedure outlined in Example 41, Step G, stirring the reaction mixture at 90° C. overnight, 2,9-difluoro-8-(trifluoromethoxy)-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (198 mg, 66% yield) was prepared as a white solid. HPLC/MS 1.15 min (B), [M+H]⁺ 561.1. ¹H NMR (DMSO-d₆, 400 MHz): δ 10.66 (br s, 1H), 9.22 (br s, 1H), 7.85-7.94 (m, 2H), 7.60 (dd, J=8.8, 1.5 Hz, 1H), 7.50 (d, J=9.8 Hz, 1H), 6.53 (d, J=5.4 Hz, 1H), 6.39 (d, J=9.8 Hz, 1H), 5.45 (d, J=11.7 Hz, 1H), 5.15 (d, J=11.2 Hz, 1H), 4.19 (br s, 2H), 3.22-3.36 (m, 1H), 3.00-3.21 (m, 2H), 2.65-2.79 (m, 1H).

### Example 53

### 2,3,8-Trifluoro-10,11,12,13-tetrahydro-19H-5,18-methanodibenzo[c,]pyrido[3,2-j][f1,5,9]triazacyclotridecine-15,19(14H)-dione hydrochloride

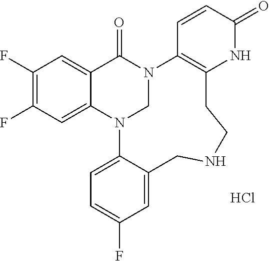 [SMILES: O=C1c2cc(F)c(F)cc2N2CN1c1ccc(=O)[nH]c1CCNCc1cc(F)ccc12 ; Cl]

### Step A: Methyl 4,5-difluoro-2-((4-fluoro-2-formylphenyl)amino)benzoate 53a

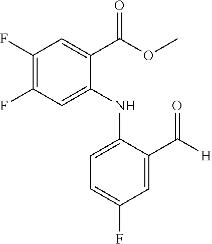 [SMILES: COC(=O)c1cc(F)c(F)cc1Nc1ccc(F)cc1C=O]

Following the preparation outlined in Example 7, Step A, substituting ethyl 2-bromo-5-(trifluoromethyl)benzoate with methyl 2-amino-4,5-difluorobenzoate, 2-bromo-5-fluorobenzaldehyde with Int-3a, BINAP with Xantphos, toluene with 1,4-dioxane, and stirring the reaction at 90° C. for 4 h, methyl 4,5-difluoro-2-((4-fluoro-2-formylphenyl)amino)benzoate 53a (10.6 g, 80% yield) was prepared as a yellow solid. HPLC/MS 1.18 min (A), [M+H]⁺ 310.0. ¹H NMR (DMSO-d₆, 400 MHz): δ 10.76 (s, 1H), 9.96 (s, 1H), 7.91 (dd, 1H, J=9.3, 11.2 Hz), 7.76 (dd, 1H, J=3.2, 8.6 Hz), 7.5-7.6 (m, 1H), 7.4-7.5 (m, 2H), 3.87 (s, 3H).

### Step B: Methyl 2-((2-(((tert-butoxycarbonyl)(2-(6-methoxy-3-nitropyridin-2-yl)ethyl-amino)methyl)-4-fluorophenyl)amino)-4,5-difluorobenzoate 53b

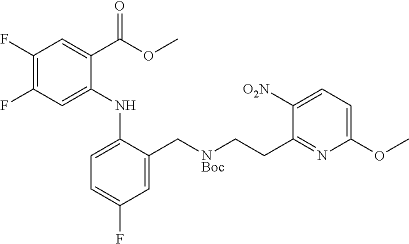 [SMILES: COC(=O)c1cc(F)c(F)cc1Nc1ccc(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4,5-difluoro-2-((4-fluoro-2-formylphenyl)amino)benzoate 53a, methyl 2-((2-(((tert-butoxycarbonyl)(2-(6-methoxy-3-nitropyridin-2-yl)ethyl)amino)methyl)-4-fluorophenyl)amino)-4,5-difluorobenzoate 53b (8.67 g, 91% yield) was prepared as a light yellow foam. HPLC/MS 1.60 min (B), [M+H]⁺ 591.1. ¹H NMR (DMSO-d₆, 400 MHz): δ 8.98 (s, 1H), 8.30 (d, 1H, J=8.8 Hz), 7.82 (dd, 1H, J=9.3, 11.2 Hz), 7.33 (dd, 1H, J=5.4, 8.8 Hz), 7.20 (dt, 1H, J=3.2, 8.4 Hz), 7.06 (br s, 1H), 6.8-6.9 (m, 1H), 6.2-6.4 (m, 1H), 4.30 (br s, 2H), 3.87 (s, 3H), 3.86 (s, 3H), 3.62 (br s, 2H), 3.15 (br s, 2H), 1.21 (s, 9H).

### Step C: 2-((2-(((tert-Butoxycarbonyl)(2-(6-methoxy-3-nitropyridin-2-yl)ethyl)amino)-methyl)-4-fluorophenyl)amino)-4,5-difluorobenzoic acid 53c

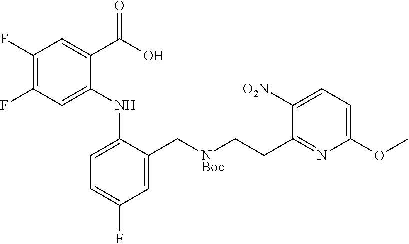 [SMILES: COc1ccc([N+](=O)[O-])c(CCN(Cc2cc(F)ccc2Nc2cc(F)c(F)cc2C(=O)O)C(=O)OC(C)(C)C)n1]

Following the procedure outlined in Example 41, Step C, stirring the reaction mixture at 50° C. for 23 h, 2-((2-(((tert-butoxycarbonyl)(2-(6-methoxy-3-nitropyridin-2-yl)ethyl)amino)-methyl)-4-fluorophenyl)amino)-4,5-difluorobenzoic acid 53c (8.50 g, 98% yield) was prepared as an off white solid. HPLC/MS 1.05 min (B), [M+H]⁺ 577.1. ¹H NMR (DMSO-d₆, 400 MHz): δ 13.36 (br s, 1H), 9.28 (br s, 1H), 8.30 (d, 1H, J=9.3 Hz), 7.80 (dd, 1H, J=9.3, 11.2 Hz), 7.34 (dd, 1H, J=5.4, 8.3 Hz), 7.19 (dt, 1H, J=3.2, 8.4 Hz), 7.04 (br s, 1H), 6.84 (br s, 1H), 6.2-6.4 (m, 1H), 4.31 (br s, 2H), 3.86 (s, 3H), 3.6-3.7 (m, 2H), 3.1-3.2 (m, 2H), 1.1-1.2 (m, 9H).

### Step D: 2-((2-(((2-(3-Amino-6-methoxypyridin-2-yl)ethyl)(tert-butoxycarbonyl)amino)-methyl)-4-fluorophenyl)amino)-4,5-difluorobenzoic acid 53d

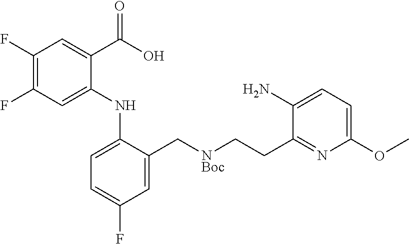 [SMILES: COc1ccc(N)c(CCN(Cc2cc(F)ccc2Nc2cc(F)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-fluorophenyl)amino)-4,5-difluorobenzoic acid 53d (4.80 g, 56% yield). HPLC/MS 0.96 min (B), [M+H]⁺ 547.2. ¹H NMR (DMSO-d₆, 400 MHz): δ 9.36 (br s, 1H), 7.80 (dd, 1H, J=9.5, 11.5 Hz), 7.33 (dd, 1H, J=5.4, 8.8 Hz), 7.17 (dt, 1H, J=3.2, 8.4 Hz), 6.9-7.1 (m, 2H), 6.2-6.4 (m, 2H), 4.29 (br s, 2H), 3.61 (s, 3H), 3.4-3.5 (m, 2H), 2.6-2.8 (m, 2H), 1.29 (br s, 9H).

### Step E: tert-Butyl 2,3,8-trifluoro-15-methoxy-19-oxo-5,10,12,13,18,19-hexahydro-11H-dibenzo[c,f]pyrido[3,2-j][1,5,9]triazacyclotridecine-11-carboxylate 53e

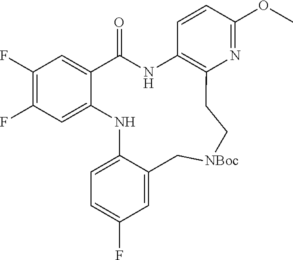 [SMILES: COc1ccc2c(n1)CCN(C(=O)OC(C)(C)C)Cc1cc(F)ccc1Nc1cc(F)c(F)cc1C(=O)N2]

Following the procedure outlined in Example 41, Step E, substituting HATU with HOAT and EDC, and stirring the reaction mixture at room temperature for 16 h, tert-butyl 2,3,8-trifluoro-15-methoxy-19-oxo-5,10,12,13,18,19-hexahydro-11H-dibenzo[c,f]pyrido[3,2-j][1,5,9]triazacyclotridecine-11-carboxylate 53e (2.32 g, 54% yield) was prepared as an off white solid. HPLC/MS 1.37 min (B), [M+H]⁺ 529.2. ¹H NMR (DMSO-d₆, 400 MHz): δ 10.40 (br s, 1H), 7.89 (br d, 2H, J=8.3 Hz), 7.54 (s, 1H), 7.22 (br d, 2H, J=6.4 Hz), 7.01 (dt, 1H, J=3.4, 8.6 Hz), 6.70 (d, 1H, J=8.8 Hz), 6.57 (br s, 1H), 4.60 (s, 2H), 3.80 (s, 3H), 3.3-3.6 (m, 2H), 2.5-2.6 (m, 2H), 1.36 (br s, 9H).

### Step F: tert-Butyl 2,3,8-trifluoro-15-methoxy-19-oxo-12,13-dihydro-19H-5,18-methano-dibenzo[c,f]pyrido[3,2-j][1,5,9]triazacyclotridecine-11(10H)-carboxylate 53f

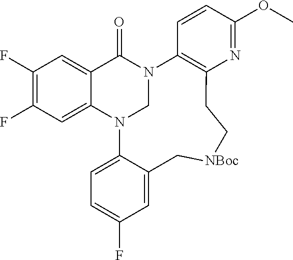 [SMILES: COc1ccc2c(n1)CCN(C(=O)OC(C)(C)C)Cc1cc(F)ccc1N1CN2C(=O)c2cc(F)c(F)cc21]

Following the procedure outlined in Example 41, Step F, stirring the reaction at 90° C. for the weekend, tert-butyl 2,3,8-trifluoro-15-methoxy-19-oxo-12,13-dihydro-19H-5,18-methano-dibenzo[c,]pyrido[3,2-j][1,5,9]triazacyclotridecine-11(10H)-carboxylate 53f (1.89 g, 84% yield) was prepared as an off white solid. HPLC/MS 1.37 min (B), [M+H]⁺ 541.2. ¹H NMR (DMSO-d₆, 400 MHz): δ 7.84 (dd, 1H, J=9.0, 10.5 Hz), 7.70 (d, 1H, J=8.3 Hz), 7.47 (dd, 1H, J=5.4, 8.8 Hz), 7.36 (dt, 1H, J=3.2, 8.4 Hz), 7.28 (dd, 1H, J=3.2, 9.0 Hz), 6.78 (d, 1H, J=8.8 Hz), 6.25 (dd, 1H, J=6.6, 12.0 Hz), 5.59 (d, 1H, J=11.7 Hz), 4.85 (d, 1H, J=11.2 Hz), 4.70 (d, 1H, J=14.2 Hz), 3.91 (br d, 1H, J=14.2 Hz), 3.85 (s, 3H), 3.5-3.7 (m, 1H), 2.9-3.0 (m, 1H), 2.5-2.7 (m, 2H), 1.45 (s, 9H).

### Step G: 2,3,8-Trifluoro-10,11,12,13-tetrahydro-19H-5,18-methanodibenzo[c,f]pyrido[3,2-f][1,5,9]triazacyclotridecine-15,19(14H)-dione hydrochloride Example 53

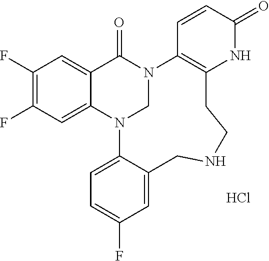 [SMILES: O=C1c2cc(F)c(F)cc2N2CN1c1ccc(=O)[nH]c1CCNCc1cc(F)ccc12 ; Cl]

Following the procedure outlined in Example 41, Step G, stirring the reaction mixture at 90° C. for 18 h, 2,3,8-trifluoro-10,11,12,13-tetrahydro-19H-5,18-methanodibenzo[c,]pyrido[3,2-j][1,5,9]triazacyclotridecine-15,19(14H)-dione hydrochloride (682 mg, 100% yield) was prepared as a white solid. HPLC/MS 0.89 min (B), [M+H]⁺ 427.2. ¹H NMR (DMSO-d₆, 400 MHz): δ 10.57 (br s, 1H), 8.94 (br s, 1H), 7.87 (dd, 1H, J=8.8, 10.8 Hz), 7.75 (dd, 1H, J=3.4, 9.3 Hz), 7.59 (dd, 1H, J=5.6, 9.0 Hz), 7.5-7.5 (m, 1H), 7.45 (d, 1H, J=9.8 Hz), 6.34 (d, 1H, J=9.8 Hz), 6.26 (dd, 1H, J=6.4, 11.7 Hz), 5.7-6.1 (m, 1H), 5.45 (d, 1H, J=11.2 Hz), 4.92 (d, 1H, J=11.7 Hz), 4.0-4.1 (m, 2H), 3.2-3.2 (m, 1H), 2.9-3.1 (m, 2H), 2.6-2.7 (m, 1H).

### Example 54

### Step A: 2,8-Difluoro-3-(methylamino)-10,11,12,13-tetrahydro-19H-5,18-methano-dibenzo[c,f]pyrido[3,2-j][1,5,9]triazacyclotridecine-15,19(14H)-dione

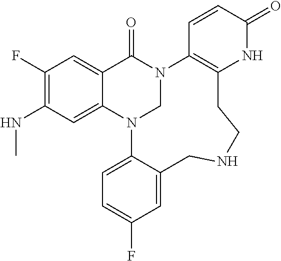 [SMILES: CNc1cc2c(cc1F)C(=O)N1CN2c2ccc(F)cc2CNCCc2[nH]c(=O)ccc21]

2,3,8-Trifluoro-10,11,12,13-tetrahydro-19H-5,18-methanodibenzo[c,f]pyrido[3,2-j][1,5,9]triazacyclotridecine-15,19(14H)-dione hydrochloride Example 53 (80.0 mg, 173 μmol) and N-methylcyclopropanamine (110.6 mg, 1.56 mmol) in DMSO (1.50 mL) were heated at 100° C. for 18 h and purified by reverse-phase semi-prep chromatography (XSELECT CSH C18 column), eluting with 30-99% AcCN in 10 mM ammonium bicarbonate in H₂O (adjusted to pH 10 with ammonia) to afford 2,8-difluoro-3-(methylamino)-10,11,12,13-tetrahydro-19H-5,18-methanodibenzo[c,f]pyrido[3,2-j][1,5,9]triazacyclotridecine-15,19(14H)-dione (11 mg, 14% yield). HPLC/MS 0.77 min (B), [M+H]⁺ 438.2. ¹H NMR (DMSO-d₆, 400 MHz): δ 11.75 (br s, 1H), 7.50 (dd, 1H, J=5.4, 8.8 Hz), 7.3-7.4 (m, 3H), 7.2-7.3 (m, 1H), 6.3-6.4 (m, 1H), 6.17 (br d, 1H, J=9.3 Hz), 5.43 (d, 1H, J=7.3 Hz), 5.16 (d, 1H, J=9.3 Hz), 4.94 (d, 1H, J=9.3 Hz), 3.85 (br t, 1H, J=11.2 Hz), 3.3-3.4 (m, 1H), 2.9-3.0 (m, 2H), 2.8-2.8 (m, 1H), 2.5-2.5 (m, 3H), 2.3-2.4 (m, 1H), 2.2-2.3 (m, 1H).

### Example 55

### 2-Chloro-8-fluoro-15,19-dioxo-10,11,12,13,14,15-hexahydro-19H-5,18-methano-dibenzo[c,f]pyrido[3,2-j][1,5,9]triazacyclotridecine-3-carbonitrile

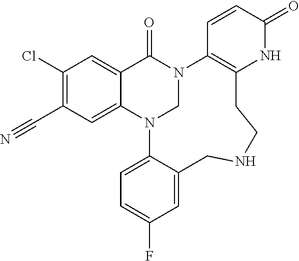 [SMILES: N#Cc1cc2c(cc1Cl)C(=O)N1CN2c2ccc(F)cc2CNCCc2[nH]c(=O)ccc21]

### Step A: tert-Butyl 2-chloro-3-cyano-8-fluoro-15-methoxy-19-oxo-12,13-dihydro-19H-5,18-methanodibenzo[c,f]pyrido[3,2-j][1,5,9]triazacyclotridecine-11(10H)-carboxylate 55a

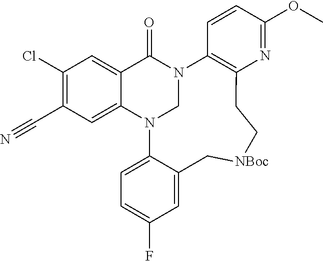 [SMILES: COc1ccc2c(n1)CCN(C(=O)OC(C)(C)C)Cc1cc(F)ccc1N1CN2C(=O)c2cc(Cl)c(C#N)cc21]

A mixture of tert-butyl 2-chloro-3,8-difluoro-15-methoxy-19-oxo-12,13-dihydro-19H-5,18-methanodibenzo[c,f]pyrido[3,2-j][1,5,9]triazacyclotridecine-11(10H)-carboxylate Example 43f (161 mg, 289 μmol), sodium cyanide (17.0 mg, 347 μmol) and tetrabutylazanium bromide (111.8 mg, 347 μmol) in DMSO (4.00 mL) was heated to 80° C. for 18 h, diluted with water and extracted with EtOAc (3×). The combined organic extracts were washed with brine, dried over MgSO₄, and filtered. The filtrate was concentrated and purified by silica gel flash column chromatography (24 g), eluting with a 100% heptanes to 50% EtOAc-heptanes gradient. Product fractions were combined and evaporated under reduced pressure to afford tert-butyl 2-chloro-3-cyano-8-fluoro-15-methoxy-19-oxo-12,13-dihydro-19H-5,18-methanodibenzo[c,f]pyrido[3,2-j][1,5,9]triazacyclotridecine-11(10H)-carboxylate 55a (129.0 mg, 75% yield) as a pale yellow solid. HPLC/MS 1.32 min (A), [M+H]⁺ 564.2. ¹H NMR (CDCl₃, 400 MHz): δ 8.22 (s, 1H), 7.48 (d, J=8.8 Hz, 1H), 7.44 (d, J=8.8 Hz, 1H), 7.16-7.20 (m, 2H), 6.69 (d, J=8.8 Hz, 1H), 6.62 (s, 1H), 5.60 (d, J=11.2 Hz, 1H), 4.86 (d, J=14.2 Hz, 1H), 4.60 (d, J=11.2 Hz, 1H), 4.00 (br d, J=14.7 Hz, 1H), 3.94-3.97 (m, 1H), 3.93 (s, 3H), 3.05 (td, J=11.7, 2.9 Hz, 1H), 2.83 (td, J=12.0, 6.8 Hz, 1H), 2.50-2.63 (m, 1H), 1.55 (s, 9H).

### Step B: 2-Chloro-8-fluoro-15,19-dioxo-10,11,12,13,14,15-hexahydro-19H-5,18-methano-dibenzo[c,f]pyrido[3,2-j][1,5,9]triazacyclotridecine-3-carbonitrile Example 55

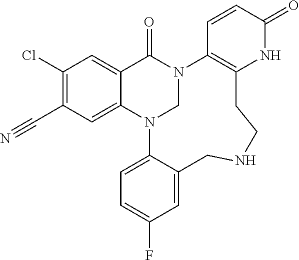 [SMILES: N#Cc1cc2c(cc1Cl)C(=O)N1CN2c2ccc(F)cc2CNCCc2[nH]c(=O)ccc21]

Following the procedure outlined in Example 41, Step G, stirring the reaction mixture at 90° C. for 8 days, 2-chloro-8-fluoro-15,19-dioxo-10,11,12,13,14,15-hexahydro-19H-5,18-methanodibenzo[c,f]pyrido[3,2-j][1,5,9]triazacyclotridecine-3-carbonitrile (63.5 mg, 59% yield) was prepared as a yellow solid. HPLC/MS 0.57 min (A), [M+H]⁺ 450.1. ¹H NMR (DMSO-d₆, 400 MHz): δ 11.84 (br s, 1H), 7.99 (s, 1H), 7.56 (dd, J=5.4, 8.8 Hz, 1H), 7.41-7.38 (m, 2H), 7.32-7.27 (m, 1H), 6.90 (s, 1H), 6.21 (br d, J=9.8 Hz, 1H), 5.38 (d, J=9.8 Hz, 1H), 5.10 (d, J=9.8 Hz, 1H), 3.84 (br d, J=10.3 Hz, 1H), 3.37-3.34 (m, 1H), 3.01-2.95 (m, 1H), 2.89-2.73 (m, 2H), 2.37 (br s, 1H), 2.22 (br d, J=13.7 Hz, 1H).

### Example 56

### 8-Fluoro-2-(2,2,2-trifluoroethoxy)-10,11,12,13-tetrahydro-19H-5,18-methanodibenzo[c,f]pyrido[3,2-j][1,5,9]triazacyclotridecine-15,19(14H)-dione hydrochloride

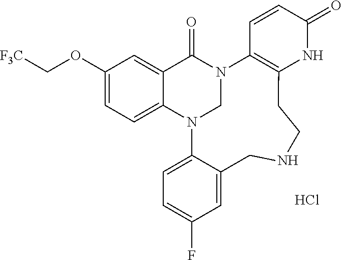 [SMILES: O=C1c2cc(OCC(F)(F)F)ccc2N2CN1c1ccc(=O)[nH]c1CCNCc1cc(F)ccc12 ; Cl]

### Step A: Methyl 2-((4-fluoro-2-formylphenyl)amino)-5-(2,2,2-trifluoroethoxy)benzoate 56a

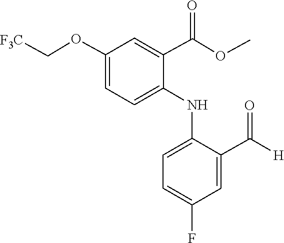 [SMILES: COC(=O)c1cc(OCC(F)(F)F)ccc1Nc1ccc(F)cc1C=O]

Following the preparation outlined in Example 7, Step A, substituting ethyl 2-bromo-5-(trifluoromethyl)benzoate with methyl 2-bromo-5-(2,2,2-trifluoroethoxy)benzoate Int-2h, Int-3a with 2-amino-5-fluorobenzaldehyde, BINAP with Xantphos, toluene with 1,4-dioxane, and stirring the reaction at 95° C. for 17 h, methyl 2-((4-fluoro-2-formylphenyl)amino)-5-(2,2,2-trifluoroethoxy)benzoate 56a (2.39 g, 67% yield) was prepared as a yellow solid. HPLC/MS 1.25 min (A), [M+H]⁺ 372.1. ¹H NMR (DMSO-d₆, 400 MHz): δ 9.94 (s, 1H), 7.71 (dd, J=8.8, 3.4 Hz, 1H), 7.52-7.57 (m, 2H), 7.37-7.44 (m, 1H), 7.32-7.34 (m, 1H), 7.29-7.32 (m, 1H), 4.82 (q, J=9.1 Hz, 2H), 3.86 (s, 3H).

### Step B: Methyl 2-((2-(((tert-butoxycarbonyl)(2-(6-methoxy-3-nitropyridin-2-yl)ethyl-amino)methyl)-4-fluorophenyl)amino)-5-(2,2,2-trifluoroethoxy)benzoate 56b

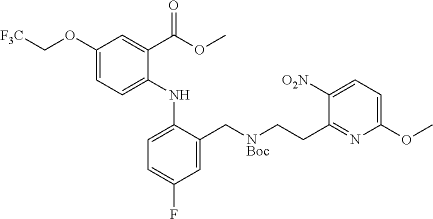 [SMILES: COC(=O)c1cc(OCC(F)(F)F)ccc1Nc1ccc(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((4-fluoro-2-formylphenyl)amino)-5-(2,2,2-trifluoroethoxy)benzoate 56a, methyl 2-((2-(((tert-butoxy-carbonyl)(2-(6-methoxy-3-nitropyridin-2-yl)ethyl)amino)methyl)-4-fluorophenyl)amino)-5-(2,2,2-trifluoroethoxy)benzoate 56b (3.83 g, 94% yield) was prepared as a yellow foam. HPLC/MS 1.58 min (A), [M+H]⁺ 653.3. ¹H NMR (DMSO-d₆, 400 MHz): δ 8.69 (s, 1H), 8.30 (br d, J=8.8 Hz, 1H), 7.49 (d, J=2.9 Hz, 1H), 7.27 (dd, J=8.8, 5.4 Hz, 1H), 7.15 (td, J=8.4, 3.2 Hz, 2H), 7.02 (br d, J=6.8 Hz, 1H), 6.80-6.88 (m, 1H), 4.70 (q, J=8.8 Hz, 2H), 4.32 (br s, 2H), 3.87 (s, 3H), 3.85 (s, 3H), 3.63 (br s, 2H), 3.10-3.19 (m, 2H), 1.21 (br s, 9H).

### Step C: 2-((2-(((tert-Butoxycarbonyl)(2-(6-methoxy-3-nitropyridin-2-yl)ethyl)amino)-methyl)-4-fluorophenyl)amino)-5-(2,2,2-trifluoroethoxy)benzoic acid hydrochloride 56c

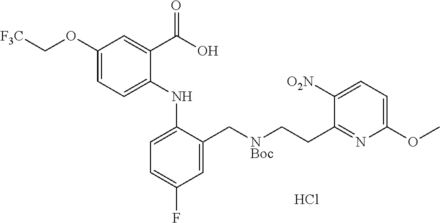 [SMILES: COc1ccc([N+](=O)[O-])c(CCN(Cc2cc(F)ccc2Nc2ccc(OCC(F)(F)F)cc2C(=O)O)C(=O)OC(C)(C)C)n1 ; Cl]

Following the procedure outlined in Example 41, Step C, stirring the reaction mixture at room temperature for 72 h, 2-((2-(((tert-butoxycarbonyl)(2-(6-methoxy-3-nitropyridin-2-yl)ethyl)amino)methyl)-4-fluorophenyl)amino)-5-(2,2,2-trifluoroethoxy)benzoic acid hydrochloride 56c (3.70 g, 96% yield) was prepared as a yellow foamy solid. HPLC/MS 1.43 min (A), [M+H]⁺ 639.2. ¹H NMR (DMSO-d₆, 400 MHz): δ 13.05-13.22 (m, 1H), 8.99 (s, 1H), 8.30 (br d, J=8.8 Hz, 1H), 7.50 (d, J=3.4 Hz, 1H), 7.29 (dd, J=8.6, 5.1 Hz, 1H), 7.08-7.18 (m, 2H), 6.99 (dd, J=9.3, 2.4 Hz, 1H), 6.86 (br d, J=13.2 Hz, 1H), 4.69 (q, J=8.8 Hz, 2H), 4.34 (br s, 2H), 3.85 (s, 3H), 3.63 (br s, 2H), 3.16 (br d, J=7.3 Hz, 2H), 1.20 (br s, 9H).

### Step D: 2-((2-(((2-(3-Amino-6-methoxypyridin-2-yl)ethyl)(tert-butoxycarbonyl)amino)-methyl)-4-fluorophenyl)amino)-5-(2,2,2-trifluoroethoxy)benzoic acid 56d

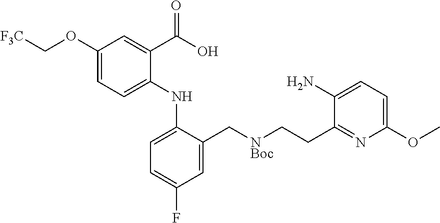 [SMILES: COc1ccc(N)c(CCN(Cc2cc(F)ccc2Nc2ccc(OCC(F)(F)F)cc2C(=O)O)C(=O)OC(C)(C)C)n1]

Following the procedure outlined in Example 41, Step D, stirring the reaction mixture for 2 h at 60° C., 2-((2-(((2-(3-amino-6-methoxypyridin-2-yl)ethyl)(tert-butoxycarbonyl)amino)-methyl)-4-fluorophenyl)amino)-5-(2,2,2-trifluoroethoxy)benzoic acid 56d (3.44 g, 100% yield) was prepared. HPLC/MS 1.07 min (A), [M+H]⁺ 609.3. ¹H NMR (DMSO-d₆, 400 MHz): δ 7.58 (d, J=2.9 Hz, 1H), 7.22 (br s, 1H), 6.88-7.06 (m, 4H), 6.84 (dd, J=9.8, 2.9 Hz, 1H), 6.35 (d, J=8.3 Hz, 1H), 4.60 (q, J=8.8 Hz, 2H), 4.39 (br s, 2H), 3.61 (s, 3H), 3.49 (br s, 2H), 2.73 (br t, J=7.1 Hz, 2H), 1.35 (br s, 9H).

### Step E: tert-Butyl 8-fluoro-15-methoxy-19-oxo-2-(2,2,2-trifluoroethoxy)-5,10,12,13,18,19-hexahydro-11H-dibenzo[c,f]pyrido[3,2-j][1,5,9]triazacyclotridecine-11-carboxylate 56e

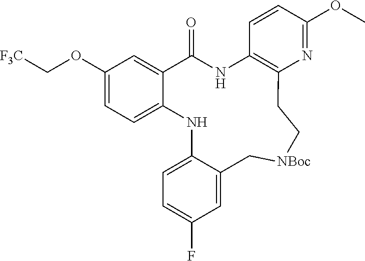 [SMILES: COc1ccc2c(n1)CCN(C(=O)OC(C)(C)C)Cc1cc(F)ccc1Nc1ccc(OCC(F)(F)F)cc1C(=O)N2]

Following the procedure outlined in Example 41, Step E, substituting HATU with pyoxim, and stirring the reaction mixture at room temperature for 2 h, tert-butyl 8-fluoro-15-methoxy-19-oxo-2-(2,2,2-trifluoroethoxy)-5,10,12,13,18,19-hexahydro-11H-dibenzo[c,f]pyrido[3,2-j][1,5,9]-triazacyclotridecine-11-carboxylate 56e (1.65 g, 50% yield) was prepared as an off white solid. HPLC/MS 1.37 min (A), [M+H]⁺ 591.3. ¹H NMR (DMSO-d₆, 400 MHz): δ 10.72-10.86 (m, 1H), 7.93 (d, J=8.8 Hz, 1H), 7.63 (br s, 1H), 7.39 (s, 1H), 7.16-7.36 (m, 3H), 6.95 (td, J=8.7, 3.2 Hz, 1H), 6.70 (d, J=8.8 Hz, 1H), 6.27 (br d, J=2.4 Hz, 1H), 4.88 (q, J=8.8 Hz, 2H), 4.67 (s, 2H), 3.80 (s, 3H), 3.43 (br s, 2H), 2.45 (br d, J=1.5 Hz, 2H), 1.30 (br s, 9H).

### Step F: tert-Butyl 8-fluoro-15-methoxy-19-oxo-2-(2,2,2-trifluoroethoxy)-12,13-dihydro-19H-5,18-methanodibenzo[c,f]pyrido[3,2-j][1,5,9]triazacyclotridecine-11(10H)-carboxylate 56f

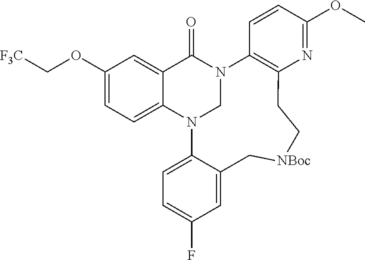 [SMILES: COc1ccc2c(n1)CCN(C(=O)OC(C)(C)C)Cc1cc(F)ccc1N1CN2C(=O)c2cc(OCC(F)(F)F)ccc21]

Following the procedure outlined in Example 41, Step F, stirring the reaction at 85° C. for 17 h, followed by addition of 5N HCl-ⁱPOH (10 equvi) and stirring at 90° C. for 100 h, followed by filtration after cooling, to afford tert-butyl 8-fluoro-15-methoxy-19-oxo-2-(2,2,2-trifluoroethoxy)-12,13-dihydro-19H-5,18-methano-dibenzo[c,f]pyrido[3,2-j][1,5,9]triazacyclo-tridecine-11(10H)-carboxylate 56f (392 mg, 79% yield) was prepared. HPLC/MS 1.40 min (A), [M+H]⁺ 603.3. ¹H NMR (DMSO-d₆, 400 MHz): δ 7.69-7.73 (m, 1H), 7.53 (d, J=2.9 Hz, 1H), 7.45 (dd, J=8.8, 5.4 Hz, 1H), 7.32-7.38 (m, 1H), 7.27 (dd, J=9.0, 3.2 Hz, 1H), 7.16 (dd, J=8.8, 2.9 Hz, 1H), 6.79 (d, J=8.8 Hz, 1H), 6.25 (d, J=9.3 Hz, 1H), 5.55 (d, J=11.7 Hz, 1H), 4.73-4.84 (m, 3H), 4.64 (br d, J=14.2 Hz, 1H), 3.89 (br d, J=14.7 Hz, 1H), 3.85 (s, 3H), 3.60-3.71 (m, 1H), 2.86-2.98 (m, 1H), 2.62-2.72 (m, 1H), 2.58 (br dd, J=11.5, 6.6 Hz, 1H), 1.45 (s, 9H).

### Step G: 8-Fluoro-2-(2,2,2-trifluoroethoxy)-10,11,12,13-tetrahydro-19H-5,18-methano-dibenzo[c,f]pyrido[3,2-j][1,5,9]triazacyclotridecine-15,19(14H)-dione hydrochloride Example 56

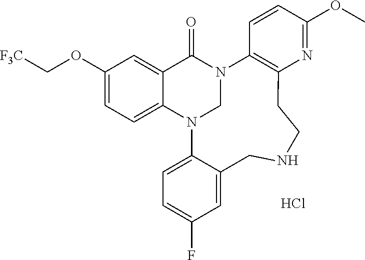 [SMILES: COc1ccc2c(n1)CCNCc1cc(F)ccc1N1CN2C(=O)c2cc(OCC(F)(F)F)ccc21 ; Cl]

Following the procedure outlined in Example 41, Step G, stirring the reaction mixture at 80° C. for 17 h, followed by the addition of 5N HCl-ⁱPrOH (10 equv) and further stirring at 90° C. for 100 h and isolation, 8-fluoro-2-(2,2,2-trifluoroethoxy)-10,11,12,13-tetrahydro-19H-5,18-methanodibenzo[c,f]pyrido[3,2-j][1,5,9]triazacyclotridecine-15,19(14H)-dione hydrochloride (295 mg, 87% yield) was prepared as a white solid. HPLC/MS 0.68 min (A), [M+H]⁺ 489.2. ¹H NMR (DMSO-d₆, 400 MHz): δ 10.57 (br d, J=5.38 Hz, 1H), 8.99 (br d, J=4.40 Hz, 1H), 7.74 (dd, J=9.29, 2.93 Hz, 1H) 7.52-7.60 (m, 2H), 7.43-7.51 (m, 2H), 7.16 (dd, J=9.05, 3.18 Hz, 1H), 6.34 (d, J=9.29 Hz, 1H), 6.23 (d, J=8.80 Hz, 1H), 5.41 (d, J=11.25 Hz, 1H), 4.89 (d, J=11.25 Hz, 1H), 4.77 (q, J=8.80 Hz, 2H), 3.98-4.10 (m, 2H), 3.15-3.26 (m, 1H), 2.88-3.06 (m, 2H), 2.63-2.73 (m, 1H).

### Example 57

### 2,8,9-Trifluoro-15,19-dioxo-10,11,12,13,14,15-hexahydro-19H-5,18-methano-dibenzo[c,f]pyrido[3,2-j][1,5,9]triazacyclotridecine-3-carbonitrile

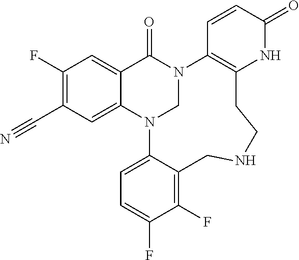 [SMILES: N#Cc1cc2c(cc1F)C(=O)N1CN2c2ccc(F)c(F)c2CNCCc2[nH]c(=O)ccc21]

### Step A: Methyl 2-((3,4-difluoro-2-formylphenyl)amino)-4,5-difluorobenzoate 57a

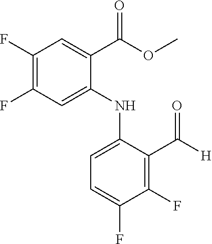 [SMILES: COC(=O)c1cc(F)c(F)cc1Nc1ccc(F)c(F)c1C=O]

Following the preparation outlined in Example 7, Step A, substituting ethyl 2-bromo-5-(trifluoromethyl)benzoate with methyl 2-amino-4,5-difluorobenzoate, Int-3a with 6-bromo-2,3-difluorobenzaldehyde, BINAP with Xantphos, toluene with 1,4-dioxane, and stirring the reaction at 80° C. over the weekend, methyl 2-((3,4-difluoro-2-formylphenyl)amino)-4,5-difluorobenzoate 57a (5.12 g, 63% yield) was prepared as a yellow solid. HPLC/MS 1.27 min (B), [M+H]⁺ 328.0. ¹H NMR (CDCl₃, 400 MHz): δ 11.33 (br s, 1H), 10.43 (s, 1H), 7.79-7.92 (m, 1H), 7.30-7.37 (m, 1H), 7.03-7.28 (m, 2H), 3.94-3.99 (m, 3H).

### Step B: Methyl 2-((2-(((tert-butoxycarbonyl)(2-(6-methoxy-3-nitropyridin-2-yl)ethyl-amino)methyl)-3,4-difluorophenyl)amino)-4-chloro-5-fluorobenzoate 49b

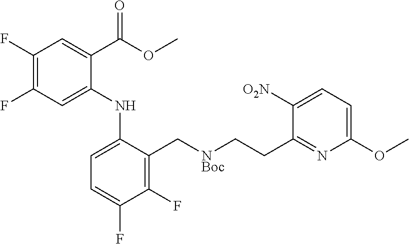 [SMILES: COC(=O)c1cc(F)c(F)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl methyl 2-((3,4-difluoro-2-formylphenyl)amino)-4,5-difluorobenzoate 57a, methyl 2-((2-(((tert-butoxy-carbonyl)(2-(6-methoxy-3-nitropyridin-2-yl)ethyl)amino)methyl)-3,4-difluorophenyl)-amino)-4,5-difluorobenzoate 57b (7.39 g, 84% yield) was prepared as a clear oil. HPLC/MS 1.62 min (B), [M+H]⁺ 609.2. ¹H NMR (CDCl₃, 400 MHz): δ 9.21 (br s, 1H), 8.23 (d, J=9.3 Hz, 1H), 7.75 (br s, 1H), 7.11-7.27 (m, 1H), 6.99-7.10 (m, 1H), 6.67 (br d, J=8.8 Hz, 1H), 6.32-6.56 (m, 1H), 4.63 (s, 2H), 3.98 (s, 3H), 3.90 (s, 3H), 3.65 (t, J=7.1 Hz, 2H), 3.28 (t, J=7.1 Hz, 2H), 1.32 (s, 9H)

### Step C: 2-((2-(((tert-Butoxycarbonyl)(2-(6-methoxy-3-nitropyridin-2-yl)ethyl)amino)-methyl)-3,4-difluorophenyl)amino)-4,5-difluorobenzoic acid 57c

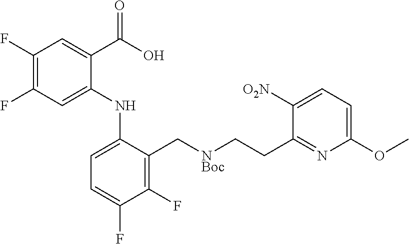 [SMILES: COc1ccc([N+](=O)[O-])c(CCN(Cc2c(Nc3cc(F)c(F)cc3C(=O)O)ccc(F)c2F)C(=O)OC(C)(C)C)n1]

Following the procedure outlined in Example 41, Step C, stirring the reaction mixture at ambient temperature for 4 h, 2-((2-(((tert-butoxycarbonyl)(2-(6-methoxy-3-nitropyridin-2-yl)ethyl)amino)methyl)-3,4-difluorophenyl)amino)-4,5-difluorobenzoic acid 57c (7.71 g, 100% yield) was prepared as a yellow foam. HPLC/MS 1.05 min (B), [M+H]⁺ 595.2. ¹H NMR (CDCl₃, 400 MHz): δ 9.26 (br s, 1H), 8.23 (d, J=8.8 Hz, 1H), 7.78 (dd, J=11.0, 9.0 Hz, 1H), 7.12-7.22 (m, 1H), 7.06 (ddd, J=9.0, 4.2, 1.5 Hz, 1H), 6.68 (br d, J=8.8 Hz, 1H), 6.47 (dd, J=13.0, 6.6 Hz, 1H), 4.59 (s, 2H), 3.95-4.01 (m, 3H), 3.71 (br t, J=7.1 Hz, 2H), 3.30 (t, J=7.1 Hz, 2H), 1.25-1.34 (m, 9H).

### Step D: 2-((2-(((2-(3-Amino-6-methoxypyridin-2-yl)ethyl)(tert-butoxycarbonyl)amino)-methyl)-3,4-difluorophenyl)amino)-4,5-difluorobenzoic acid 57d

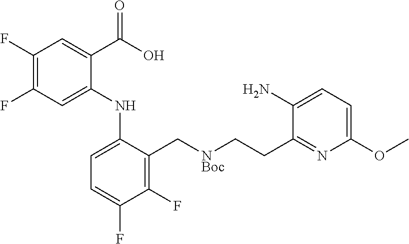 [SMILES: COc1ccc(N)c(CCN(Cc2c(Nc3cc(F)c(F)cc3C(=O)O)ccc(F)c2F)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-3,4-difluorophenyl)amino)-4,5-difluorobenzoic acid 57d (6.97 g, 92% yield) was prepared as a gray solid. HPLC/MS 0.94 min (B), [M+H]⁺ 565.2. ¹H NMR (CDCl₃, 400 MHz): δ 9.03-9.25 (m, 1H), 7.81 (dd, J=11.0, 9.0 Hz, 1H), 7.03-7.28 (m, 2H), 6.94 (br dd, J=8.1, 3.2 Hz, 1H), 6.33-6.52 (m, 2H), 4.51 (s, 2H), 3.73-3.84 (m, 3H), 3.37-3.60 (m, 2H), 2.92 (br s, 2H), 1.36-1.52 (m, 9H).

### Step E: tert-Butyl 2,3,8,9-tetrafluoro-15-methoxy-19-oxo-5,10,12,13,18,19-hexahydro-11H-dibenzo[c,f]pyrido[3.2-j][1.5.9]triazacyclotridecine-11-carboxylate 57e

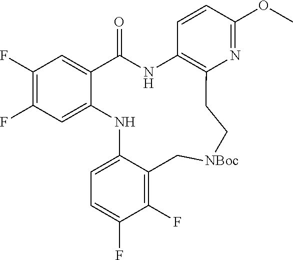 [SMILES: COc1ccc2c(n1)CCN(C(=O)OC(C)(C)C)Cc1c(ccc(F)c1F)Nc1cc(F)c(F)cc1C(=O)N2]

Following the procedure outlined in Example 41, Step E, stirring the reaction mixture at room temperature for 1 h, tert-butyl 2,3,8,9-tetrafluoro-15-methoxy-19-oxo-5,10,12,13,18,19-hexahydro-11H-dibenzo[c,f]pyrido[3,2-j][1,5,9]triazacyclotridecine-11-carboxylate 57e (4.75 g, 69% yield) was prepared as a white solid. HPLC/MS 1.41 min (B), [M+H]⁺ 547.2. ¹H NMR (CDCl₃, 400 MHz): δ 10.69 (br s, 1H), 8.05-8.22 (m, 2H), 7.00 (dd, J=10.3, 6.8 Hz, 1H), 6.84 (q, J=9.3 Hz, 1H), 6.49 (d, J=9.3 Hz, 1H), 6.24 (br s, 1H), 5.88 (br dd, J=9.0, 2.2 Hz, 1H), 3.73 (s, 3H), 2.05-2.33 (m, 2H), 1.46 (s, 4H), 0.91 (s, 9H).

### Step F: tert-Butyl 2,3,8,9-tetrafluoro-15-methoxy-19-oxo-12,13-dihydro-19H-5,18-methanodibenzo[c,f]pyrido[3,2-j][1,5,9]triazacyclotridecine-11(10H)-carboxylate 57

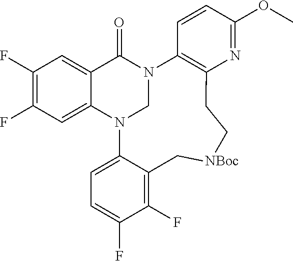 [SMILES: COc1ccc2c(n1)CCN(C(=O)OC(C)(C)C)Cc1c(ccc(F)c1F)N1CN2C(=O)c2cc(F)c(F)cc21]

Following the procedure outlined in Example 41, Step F, stirring the reaction at 90° C. for 7 h, tert-butyl 2,3,8,9-tetrafluoro-15-methoxy-19-oxo-12,13-dihydro-19H-5,18-methanodibenzo-[c,f]pyrido[3,2-j][1,5,9]triazacyclotridecine-11(10H)-carboxylate 57f (1.93 g, 39% yield) was prepared as a light tan solid. HPLC/MS 1.36 min (B), [M+H]⁺ 559.2. ¹H NMR (CDCl₃, 400 MHz): δ 7.79 (dd, J=10.3, 8.8 Hz, 1H), 7.32 (d, J=8.3 Hz, 1H), 7.14-7.18 (m, 2H), 6.79-6.92 (m, 1H), 6.52, (d, J=8.3 Hz, 1H), 5.95 (dd, J=11.2, 6.4 Hz, 1H), 5.43 (d, J=10.8 Hz, 1H), 5.03 (br d, J=14.7 Hz, 1H), 4.39 (d, J=11.2 Hz, 1H), 3.72-3.92 (m, 4H), 2.92 (td, J=11.5, 3.4 Hz, 1H), 2.70 (td, J=11.6, 7.6 Hz, 1H), 2.30-2.51 (m, 1H), 1.36-1.47 (m, 9H).

### Step G: tert-Butyl 3-cyano-2,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 57g

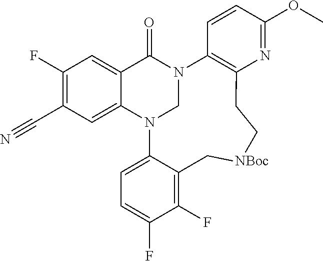 [SMILES: COc1ccc2c(n1)CCN(C(=O)OC(C)(C)C)Cc1c(ccc(F)c1F)N1CN2C(=O)c2cc(F)c(C#N)cc21]

A mixture of tert-butyl 2,3,8,9-tetrafluoro-15-methoxy-19-oxo-12,13-dihydro-19H-5,18-methanodibenzo[c,f]pyrido[3,2-j][1,5,9]triazacyclotridecine-11(10H)-carboxylate 57g, sodium cyanide (15.8 mg, 322 μmol) and tetrabutylammonium bromide (104 mg, 322 μmol) in DMSO (4.00 mL) was heated to 80° C. overnight, diluted with water and extracted with EtOAc (3×). The combined organic extracts were washed brine, dried over MgSO₄, filtered, and evaporated under reduced pressure. The crude residue was dissolved in DCM, adsorbed on a silica gel pre-column and purified by silica gel flash column chromatography (40 g), eluting with a 100% hetpanes to 50% EtOAcheptanes gradient. Product fractions were combined and evaporated under reduced pressure to afford tert-butyl 3-cyano-2,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 57f (89 mg, 39% yield) as a pale yellow solid. HPLC/MS 1.34 min (B), [M+H]⁺ 566.2. ¹H NMR (DMSO-d₆, 400 MHz): δ 7.88 (d, J=9.3 Hz, 1H), 7.72 (d, J=8.3 Hz, 1H), 7.54-7.68 (m, 1H), 7.32 (ddd, J=6.6, 4.4, 2.2 Hz, 1H), 6.95 (d, J=4.9 Hz, 1H), 6.80 (d, J=8.2 Hz, 1H), 5.62 (d, J=11.7 Hz, 1H), 5.02 (br d, J=14.7 Hz, 1H), 4.88 (br d, J=11.2 Hz, 1H), 3.79-3.95 (m, 4H), 3.63-3.79 (m, 1H), 2.85-2.96 (m, 1H), 2.53-2.77 (m, 2H), 1.29-1.52 (m, 9H).

### Step H: 2,8,9-Trifluoro-15,19-dioxo-10,11,12,13,14,15-hexahydro-19H-5,18-methano-dibenzo[c,f]pyrido[3,2-j][1,5,9]triazacyclotridecine-3-carbonitrile

### Example 57

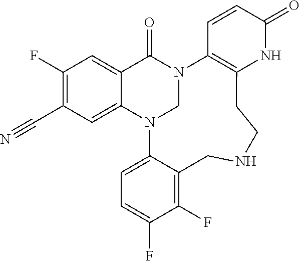 [SMILES: N#Cc1cc2c(cc1F)C(=O)N1CN2c2ccc(F)c(F)c2CNCCc2[nH]c(=O)ccc21]

Following the procedure outlined in Example 41, Step G, stirring the reaction mixture at 90° C. overnight, 2,8,9-trifluoro-15,19-dioxo-10,11,12,13,14,15-hexahydro-19H-5,18-methano-dibenzo[c,f]pyrido[3,2-j][1,5,9]triazacyclotridecine-3-carbonitrile (22 mg, 30% yield) was prepared. HPLC/MS 0.86 min (B), [M+H]⁺ 452.2. ¹H NMR (DMSO-d₆, 400 MHz): δ 7.85 (br d, J=8.8 Hz, 1H), 7.53-7.62 (m, 1H), 7.36-7.51 (m, 2H), 6.97 (br s, 1H), 6.23 (br d, J=9.3 Hz, 1H), 5.39 (br d, J=9.8 Hz, 1H), 5.05 (br d, J=9.8 Hz, 1H), 3.68 (br s, 2H), 2.78-3.05 (m, 3H), 2.17-2.29 (m, 1H).

### Example 58

### 9-Chloro-2,8-difluoro-3-(trifluoromethyl)-10,11,12,13-tetrahydro-19H-5,18-methano-dibenzo[c,f]pyrido[3,2-j][1,5,9]triazacyclotridecine-15,19(14H)-dione

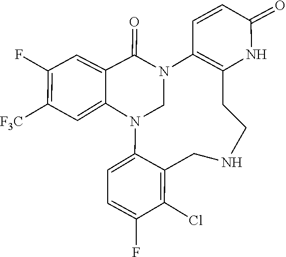 [SMILES: O=C1c2cc(F)c(C(F)(F)F)cc2N2CN1c1ccc(=O)[nH]c1CCNCc1c2ccc(F)c1Cl]

### Step A: Methyl 2-((3-chloro-4-fluoro-2-formylphenyl)amino)-5-fluoro-4-(trifluoro-methyl)benzoate 58a

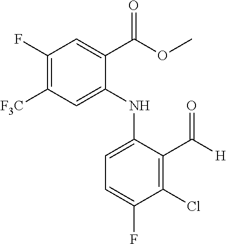 [SMILES: COC(=O)c1cc(F)c(C(F)(F)F)cc1Nc1ccc(F)c(Cl)c1C=O]

Following the preparation outlined in Example 7, Step A, substituting ethyl 2-bromo-5-(trifluoromethyl)benzoate with methyl 2-amino-5-fluoro-4-(trifluoromethyl)benzoate, Int-3a with 6-bromo-2-chloro-3-fluorobenzaldehyde, BINAP with Xantphos, toluene with 1,4-dioxane, and stirring the reaction at 80° C. for 2 h, methyl 2-((3-chloro-4-fluoro-2-formylphenyl)amino)-5-fluoro-4-(trifluoromethyl)benzoate 58a (988 mg, 77% yield) was prepared as a pale yellow solid. HPLC/MS 1.37 min (A), [M+H]⁺ 394.0. ¹H NMR (CDCl₃, 400 MHz): δ 11.44 (s, 1H), 10.59 (s, 1H), 7.86 (d, J=10.8 Hz, 1H), 7.74 (d, J=5.9 Hz, 1H), 7.21-7.35 (m, 4H), 4.01 (s, 3H).

### Step B: Methyl 2-((2-(((tert-butoxycarbonyl)(2-(6-methoxy-3-nitropyridin-2-yl)ethyl-amino)methyl)-3-chloro-4-fluorophenyl)amino)-5-fluoro-4-(trifluoromethyl)benzoate 58b

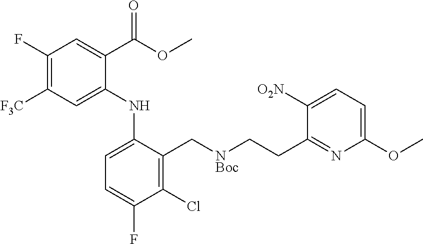 [SMILES: COC(=O)c1cc(F)c(C(F)(F)F)cc1Nc1ccc(F)c(Cl)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3-chloro-4-fluoro-2-formylphenyl)amino)-5-fluoro-4-(trifluoromethyl)benzoate 58a, methyl 2-((2-(((tert-butoxycarbonyl)(2-(6-methoxy-3-nitropyridin-2-yl)ethyl)amino)methyl)-3-chloro-4-fluoro-phenyl)amino)-5-fluoro-4-(trifluoromethyl)benzoate 58b (1.95 g, 80% yield) was prepared as a pale yellow solid. HPLC/MS 1.67 min (A), [M+H]⁺ 675.2. ¹H NMR (CDCl₃, 400 MHz): δ 1.34 (s, 9H) 3.32 (t, J=7.09 Hz, 2H), 3.65 (t, J=7.09 Hz, 2H), 3.89-4.06 (m, 6H), 4.80 (s, 2H), 6.69 (br d, J=8.80 Hz, 1H), 6.91 (br d, J=5.87 Hz, 1H), 7.14-7.26 (m, 2H), 7.65-7.91 (m, 1H), 8.24 (d, J=8.80 Hz, 1H), 9.22 (br s, 1H).

### Step C: 2-((2-(((tert-Butoxycarbonyl)(2-(6-methoxy-3-nitropyridin-2-yl)ethyl)amino)-methyl)-3-chloro-4-fluorophenyl)amino)-5-fluoro-4-(trifluoromethyl)benzoic acid 58c

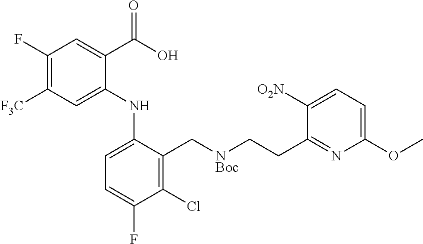 [SMILES: COc1ccc([N+](=O)[O-])c(CCN(Cc2c(Nc3cc(C(F)(F)F)c(F)cc3C(=O)O)ccc(F)c2Cl)C(=O)OC(C)(C)C)n1]

Following the procedure outlined in Example 41, Step C, stirring the reaction mixture at ambient temperature for 17 h, 2-((2-(((tert-butoxycarbonyl)(2-(6-methoxy-3-nitropyridin-2-yl)ethyl)amino)methyl)-3-chloro-4-fluorophenyl)amino)-5-fluoro-4-(trifluoromethyl)benzoic acid 58c (1.96 g, 100% yield) was prepared as a light yellow foam. HPLC/MS 1.51 min (A), [M+H]⁺ 661.2. ¹H NMR (CDCl₃, 400 MHz): δ 1.22-1.40 (m, 6H), 3.34 (br t, J=7.09 Hz, 2H), 3.67 (br t, J=7.09 Hz, 2H), 4.00 (s, 2H), 4.80 (s, 2H), 6.70 (br d, J=9.29 Hz, 1H), 6.86-6.97 (m, 1H), 7.21 (d, J=6.85 Hz, 1H), 7.75-7.91 (m, 1H), 8.25 (d, J=8.80 Hz, 2H), 9.19 (s, 1H).

### Step D: 2-((2-(((2-(3-Amino-6-methoxypyridin-2-yl)ethyl)(tert-butoxycarbonyl)amino)-methyl)-3-chloro-4-fluorophenyl)amino)-5-fluoro-4-(trifluoromethyl) benzoic acid 58d

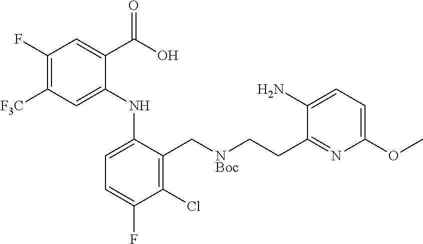 [SMILES: COc1ccc(N)c(CCN(Cc2c(Nc3cc(C(F)(F)F)c(F)cc3C(=O)O)ccc(F)c2Cl)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-3-chloro-4-fluorophenyl)-amino)-5-fluoro-4-(trifluoromethyl)benzoic acid 58d (1.05 g, 90% yield) was prepared as a brown foam. HPLC/MS 1.20 min (A), [M+H]⁺ 631.2. ¹H NMR (CDCl₃, 400 MHz): δ 1.41-1.51 (m, 9H), 3.09 (br s, 2H), 3.42 (br t, J=7.09 Hz, 2H), 3.81 (s, 3H), 4.70 (s, 2H), 6.54 (d, J=8.80 Hz, 1H), 6.96-7.07 (m, 1H), 7.14-7.19 (m, 2H), 7.38-7.54 (m, 1H), 7.87 (br d, J=11.25 Hz, 2H), 9.05-9.43 (m, 1H).

### Step E: tert-Butyl 9-chloro-2,8-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 58e

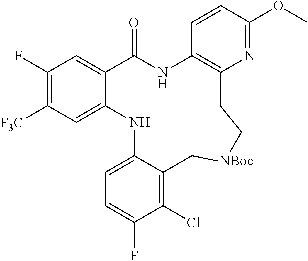 [SMILES: COc1ccc2c(n1)CCN(C(=O)OC(C)(C)C)Cc1c(ccc(F)c1Cl)Nc1cc(C(F)(F)F)c(F)cc1C(=O)N2]

Following the procedure outlined in Example 41, Step E, stirring the reaction mixture at room temperature for 1.5 h, tert-butyl 9-chloro-2,8-difluoro-15-methoxy-19-oxo-3-(trifluoromethyl)-5,10,12,13,18,19-hexahydro-11H-dibenzo[c,f]pyrido[3,2-j][1,5,9]triazacyclo-tridecine-11-carboxylate 58e (1.36 g, 74% yield) was prepared as a white solid. HPLC/MS 1.50 min (A), [M+H]⁺ 613.1. ¹H NMR (CDCl₃, 400 MHz): δ 1.08 (s, 9H), 2.09 (s, 1H), 3.24-3.70 (m, 1H), 3.90 (s, 3H), 4.03-4.48 (m, 1H), 5.34 (s, 1H), 6.18 (dd, J=8.80, 4.40 Hz, 1H), 6.55 (br s, 1H), 6.67 (d, J=8.80 Hz, 1H), 7.03 (t, J=8.56 Hz, 1H), 7.59 (d, J=5.87 Hz, 1H), 8.21-8.43 (m, 2H), 10.83-11.15 (m, 1H).

### Step F: tert-Butyl 9-chloro-2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 58f

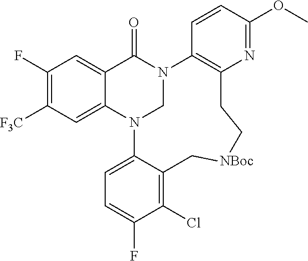 [SMILES: COc1ccc2c(n1)CCN(C(=O)OC(C)(C)C)Cc1c(ccc(F)c1Cl)N1CN2C(=O)c2cc(F)c(C(F)(F)F)cc21]

Following the procedure outlined in Example 41, Step F, stirring the reaction at 80° C. for 72 h, tert-butyl 9-chloro-2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 58f (973 mg, 69% yield) was prepared as a white solid. HPLC/MS 1.46 min (A), [M+H]⁺ 625.2. ¹H NMR (CDCl₃, 400 MHz): δ 1.54 (br s, 8H), 2.30-2.59 (m, 1H), 2.80-3.03 (m, 1H), 3.04 (br d, J=9.29 Hz, 1H), 3.95 (s, 3H), 4.02-4.36 (m, 2H), 4.55 (d, J=11.25 Hz, 1H), 5.02-5.27 (m, 1H), 5.60 (d, J=10.76 Hz, 1H), 6.46-6.57 (m, 1H), 6.70 (d, J=8.80 Hz, 1H), 7.12-7.20 (m, 1H), 7.31 (s, 10H), 7.50 (d, J=8.31 Hz, 1H), 7.98 (d, J=9.78 Hz, 1H).

### Step G: 9-Chloro-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione Example 58

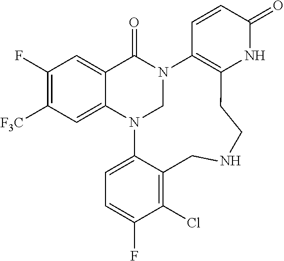 [SMILES: O=C1c2cc(F)c(C(F)(F)F)cc2N2CN1c1ccc(=O)[nH]c1CCNCc1c2ccc(F)c1Cl]

Following the procedure outlined in Example 41, Step G, stirring the reaction mixture at 90° C. for 17 h, 9-chloro-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione (389 mg, 89% yield) was prepared as a light yellow solid. HPLC/MS 0.68 min (A), [M+H]⁺ 511.1. ¹H NMR (DMSO-d₆, 400 MHz): δ 2.23-2.40 (m, 2H), 2.76-3.15 (m, 3H), 3.69 (br d, J=8.80 Hz, 2H), 3.91-4.02 (m, 1H), 5.05 (d, J=9.78 Hz 2H), 5.39 (d, J=9.78 Hz, 2H), 6.23 (br s, 2H), 6.69 (d, J=5.38 Hz, 2H), 7.35-7.49 (m, 2H), 7.49-7.61 (m, 2H), 7.67 (dd, J=8.80, 4.89 Hz, 2H), 7.80-7.92 (m, 2H), 11.88 (br s, 1H).

### Example 59

### 2-Chloro-8-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

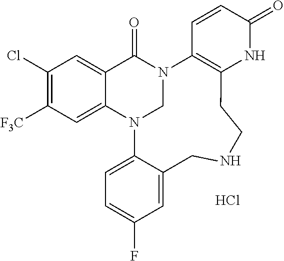 [SMILES: O=C1c2cc(Cl)c(C(F)(F)F)cc2N2CN1c1ccc(=O)[nH]c1CCNCc1cc(F)ccc12 ; Cl]

### Step A: Methyl 5-chloro-2-((4-fluoro-2-formylphenyl)amino)-4-(trifluoromethyl)-benzoate 59a

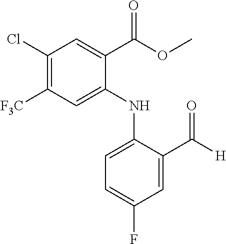 [SMILES: COC(=O)c1cc(Cl)c(C(F)(F)F)cc1Nc1ccc(F)cc1C=O]

Following the preparation outlined in Example 7, Step A, substituting ethyl 2-bromo-5-(trifluoromethyl)benzoate with methyl 2-amino-5-chloro-4-(trifluoromethyl)benzoate Int-2i, Int-3a with 2-bromo-5-fluorobenzaldehyde, BINAP with Xantphos, toluene with 1,4-dioxane, and stirring the reaction at 80° C. for 28 h, methyl 5-chloro-2-((4-fluoro-2-formylphenyl)amino)-4-(trifluoromethyl)benzoate 59a (1.32 g, 45% yield) was prepared as a yellow solid. HPLC/MS 1.34 min (A), [M+H]⁺ 376.0. ¹H NMR (CDCl₃, 400 MHz): δ 11.09 (s, 1H), 10.03 (s, 1H), 8.16 (s, 1H), 7.81 (s, 1H), 7.48-7.50 (m, 2H), 7.29-7.31 (m, 1H), 4.04 (s, 3H).

### Step B: Methyl 2-((2-(((tert-butoxycarbonyl)(2-(6-methoxy-3-nitropyridin-2-yl)ethyl)-amino)methyl)-4-fluorophenyl)amino)-5-chloro-4-(trifluoromethyl)benzoate 59b

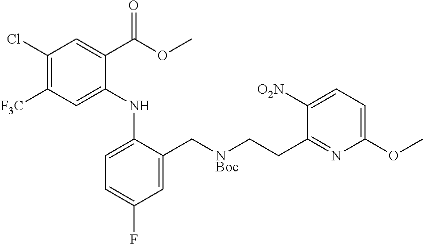 [SMILES: COC(=O)c1cc(Cl)c(C(F)(F)F)cc1Nc1ccc(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-chloro-2-((4-fluoro-2-formylphenyl)amino)-4-(trifluoromethyl)benzoate 59a, methyl 2-((2-(((tert-butoxy-carbonyl)(2-(6-methoxy-3-nitropyridin-2-yl)ethyl)amino)methyl)-4-fluorophenyl)amino)-5-chloro-4-(trifluoromethyl)benzoate 59b (1.95 g, 80% yield) was prepared as a yellow solid. HPLC/MS 1.63 min (A), [M+H]⁺ 657.2. ¹H NMR (CDCl₃, 400 MHz): δ 9.22 (s, 1H), 8.23 (d, 1H, J=8.8 Hz), 8.08 (s, 1H), 7.2-7.3 (m, 1H), 7.07 (s, 2H), 6.8-7.0 (m, 1H), 6.6-6.7 (m, 1H), 4.3-4.7 (m, 2H), 3.9-4.1 (m, 6H), 3.6-3.8 (m, 2H), 3.30 (br s, 2H), 1.40 (s, 9H).

### Step C: 2-((2-(((tert-Butoxycarbonyl)(2-(6-methoxy-3-nitropyridin-2-yl)ethyl)amino)-methyl)-3-chloro-4-fluorophenyl)amino)-5-fluoro-4-(trifluoromethyl)benzoic acid 59c

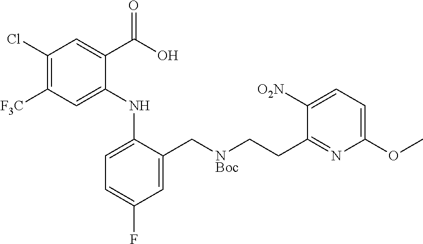 [SMILES: COc1ccc([N+](=O)[O-])c(CCN(Cc2cc(F)ccc2Nc2cc(C(F)(F)F)c(Cl)cc2C(=O)O)C(=O)OC(C)(C)C)n1]

Following the procedure outlined in Example 41, Step C, stirring the reaction mixture at 50° C. for 7 h, then at 23° C. for 13.5 h, 2-((2-(((tert-butoxycarbonyl)(2-(6-methoxy-3-nitropyridin-2-yl)ethyl)amino)methyl)-4-fluorophenyl)amino)-5-chloro-4-(trifluoromethyl)benzoic acid 59c (1.85 g, 100% yield) was prepared as a yellow foam. HPLC/MS 1.53 min (A), [M+H]⁺ 643.2. ¹H NMR (DMSO-d₆, 400 MHz): δ 11.6-12.2 (m, 1H), 8.29 (br s, 1H), 7.99 (s, 1H), 7.2-7.5 (m, 1H), 7.12 (br d, 1H, J=2.9 Hz), 6.93 (br s, 1H), 6.7-6.9 (m, 1H), 4.39 (br s, 2H), 3.82 (s, 3H), 3.6-3.8 (m, 2H), 3.34 (s, 4H), 3.1-3.2 (m, 2H), 1.0-1.4 (m, 10H).

### Step D: 2-((2-(2-(-(3-Amino-6-methoxypyridin-2-yl)ethyl)(tert-butoxycarbonyl)amino)-methyl)-4-fluorophenyl)amino)-5-chloro-4-(trifluoromethyl)benzoic acid 59d

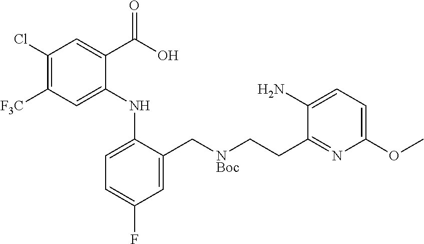 [SMILES: COc1ccc(N)c(CCN(Cc2cc(F)ccc2Nc2cc(C(F)(F)F)c(Cl)cc2C(=O)O)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-4-fluorophenyl)amino)-5-chloro-4-(trifluoromethyl)benzoic acid 59d (1.68 g, 97% yield) was prepared as a light yellow foam. HPLC/MS 1.15 min (A), [M+H]⁺ 613.2. ¹H NMR (DMSO-d₆, 400 MHz): δ 9.40 (s, 1H), 7.99 (br s, 1H), 7.37 (br d, 1H, J=5.4 Hz), 7.22 (br d, 2H, J=2.9 Hz), 6.96 (d, 1H, J=8.3 Hz), 6.71 (s, 1H), 6.37 (d, 1H, J=8.3 Hz), 4.30 (br s, 2H), 3.61 (s, 3H), 3.3-3.5 (m, 2H), 2.71 (br t, 2H, J=6.8 Hz), 1.1-1.4 (m, 11H).

### Step E: tert-Butyl 2-chloro-8-fluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 59e

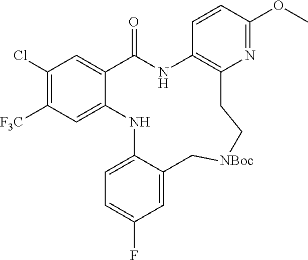 [SMILES: COc1ccc2c(n1)CCN(C(=O)OC(C)(C)C)Cc1cc(F)ccc1Nc1cc(C(F)(F)F)c(Cl)cc1C(=O)N2]

Following the procedure outlined in Example 41, Step E, stirring the reaction mixture at room temperature for 96 h, tert-butyl 2-chloro-8-fluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 59e (916.6 mg, 55% yield) was prepared as a white solid. HPLC/MS 1.42 min (A), [M+H]⁺ 595.1. ¹H NMR (CDCl₃, 400 MHz): δ 10.7-11.1 (m, 1H), 8.58 (s, 1H), 8.21 (d, 1H, J=8.8 Hz), 7.63 (s, 1H), 7.0-7.1 (m, 1H), 6.9-7.0 (m, 1H), 6.66 (d, 1H, J=8.8 Hz), 6.3-6.4 (m, 1H), 6.3-6.3 (m, 1H), 4.5-4.9 (m, 1H), 3.90 (s, 3H), 3.7-3.8 (m, 2H), 2.2-2.5 (m, 2H), 1.11 (s, 10H).

### Step F: tert-Butyl 9-chloro-2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 59f

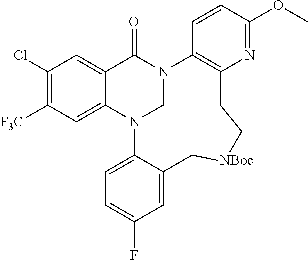 [SMILES: COc1ccc2c(n1)CCN(C(=O)OC(C)(C)C)Cc1cc(F)ccc1N1CN2C(=O)c2cc(Cl)c(C(F)(F)F)cc21]

Following the procedure outlined in Example 41, Step F, stirring the reaction at 90° C. for 20.5 h, tert-butyl 2-chloro-8-fluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 59f (864.5 mg, 90% yield) was prepared as a white solid. HPLC/MS 1.44 min (A), [M+H]⁺ 607.1. ¹H NMR (CDCl₃, 400 MHz): δ 8.1-8.3 (m, 1H), 7.50 (s, 1H), 7.4-7.5 (m, 1H), 7.1-7.3 (m, 2H), 6.71 (d, 1H, J=8.3 Hz), 6.64 (s, 1H), 5.62 (d, 1H, J=11.2 Hz), 4.8-4.9 (m, 1H), 4.62 (d, 1H, J=11.2 Hz), 4.0-4.1 (m, 1H), 3.94 (s, 4H), 3.0-3.2 (m, 1H), 2.8-2.9 (m, 1H), 2.5-2.7 (m, 1H), 1.57 (s, 9H).

### Step G: 2-Chloro-8-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 59

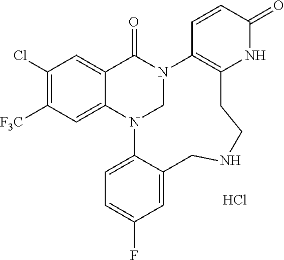 [SMILES: O=C1c2cc(Cl)c(C(F)(F)F)cc2N2CN1c1ccc(=O)[nH]c1CCNCc1cc(F)ccc12 ; Cl]

Following the procedure outlined in Example 41, Step G, stirring the reaction mixture at 90° C. for 24 h, 2-chloro-8-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (560 mg, 72% yield) was prepared as a white solid. HPLC/MS 0.72 min (A), [M+H]⁺ 493.1. ¹H NMR (CD₃OD, 400 MHz): δ 8.20 (s, 1H), 7.6-7.7 (m, 3H), 7.52 (br dd, 1H, J=2.9, 8.3 Hz), 6.4-6.7 (m, 2H), 5.63 (d, 1H, J=11.2 Hz), 4.9-5.0 (m, 2H), 4.45 (d, 1H, J=13.2 Hz), 4.23 (br d, 1H, J=13.2 Hz), 3.4-3.5 (m, 1H), 3.2-3.3 (m, 1H), 3.1-3.2 (m, 1H), 2.7-2.9 (m, 1H).

### Example 60

### 2-Chloro-8,9-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

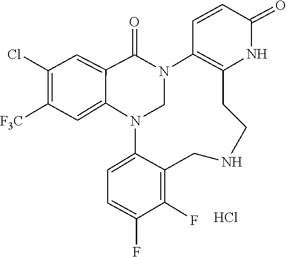 [SMILES: O=C1c2cc(Cl)c(C(F)(F)F)cc2N2CN1c1ccc(=O)[nH]c1CCNCc1c2ccc(F)c1F ; Cl]

### Step A: Methyl 5-chloro-2-((3,4-difluoro-2-formylphenyl)amino)-4-(trifluoromethyl)-benzoate 60a

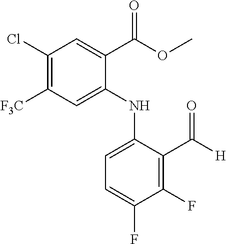 [SMILES: COC(=O)c1cc(Cl)c(C(F)(F)F)cc1Nc1ccc(F)c(F)c1C=O]

Following the preparation outlined in Example 7, Step A, substituting ethyl 2-bromo-5-(trifluoromethyl)benzoate with methyl 2-amino-5-chloro-4-(trifluoromethyl)benzoate, Int-3a with 2-bromo-5,6-difluorobenzaldehyde, BINAP with Xantphos, toluene with 1,4-dioxane, and stirring the reaction at 80° C. for 24 h, methyl 5-chloro-2-((3,4-difluoro-2-formylphenyl)amino)-4-(trifluoromethyl)benzoate 60a (1.40 g, 44% yield) was prepared as a yellow solid. HPLC/MS 1.36 min (A), [M+H]⁺ 394.0. ¹H NMR (CDCl₃, 400 MHz): δ 11.42 (s, 1H), 10.46 (d, 1H, J=1.0 Hz), 8.16 (s, 1H), 7.90 (s, 1H), 7.38 (d, 1H, J=9.3 Hz), 7.1-7.2 (m, 1H), 4.04 (s, 3H).

### Step B: Methyl 2-((2-(((tert-butoxycarbonyl)(2-(6-methoxy-3-nitropyridin-2-yl)ethyl-amino)methyl)-3,4-difluorophenyl)amino)-5-chloro-4-(trifluoromethyl)benzoate 60b

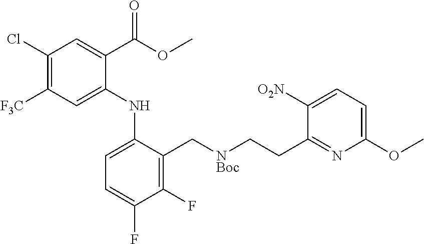 [SMILES: COC(=O)c1cc(Cl)c(C(F)(F)F)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-chloro-2-((3,4-difluoro-2-formylphenyl)amino)-4-(trifluoromethyl)benzoate 60a, methyl 2-((2-(((tert-butoxycarbonyl)(2-(6-methoxy-3-nitropyridin-2-yl)ethyl)amino)methyl)-3,4-difluorophenyl)-amino)-5-chloro-4-(trifluoromethyl)benzoate 60b (1.86 g, 75% yield) was prepared as a yellow solid. HPLC/MS 1.63 min (A), [M+H]⁺ 675.2. ¹H NMR (CDCl₃, 400 MHz): δ 9.36 (s, 1H), 8.2-8.3 (m, 1H), 8.06 (s, 1H), 7.1-7.2 (m, 1H), 7.03 (s, 2H), 6.6-6.8 (m, 1H), 4.63 (d, 2H, J=1.5 Hz), 3.9-4.0 (m, 6H), 3.70 (s, 2H), 3.32 (t, 2H, J=7.1 Hz), 1.33 (s, 11H).

### Step C: 2-((2-(((tert-Butoxycarbonyl)(2-(6-methoxy-3-nitropyridin-2-yl)ethyl)amino)-methyl)-3,4-difluorophenyl)amino)-5-chloro-4-(trifluoromethyl)benzoic acid 60c

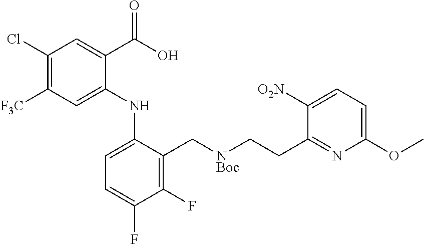 [SMILES: COc1ccc([N+](=O)[O-])c(CCN(Cc2c(Nc3cc(C(F)(F)F)c(Cl)cc3C(=O)O)ccc(F)c2F)C(=O)OC(C)(C)C)n1]

Following the procedure outlined in Example 41, Step C, stirring the reaction mixture at 50° C. for 3 h, 2-((2-(((tert-butoxycarbonyl)(2-(6-methoxy-3-nitropyridin-2-yl)ethyl)amino)-methyl)-3,4-difluorophenyl)amino)-5-chloro-4-(trifluoromethyl)benzoic acid 60c (1.85 g, 100% yield) was prepared as a yellow foam. HPLC/MS 1.54 min (A), [M+H]⁺ 661.2. ¹H NMR (DMSO-d₆, 400 MHz): δ 13.4-14.2 (m, 1H), 9.50 (s, 1H), 8.33 (br s, 1H), 7.98 (s, 1H), 7.48 (d, 1H, J=9.8 Hz), 7.25 (br dd, 1H, J=4.2, 8.6 Hz), 6.83 (s, 2H), 4.52 (br s, 2H), 3.91 (br s, 3H), 3.55 (br t, 2H, J=6.6 Hz), 3.14 (br t, 2H, J=6.1 Hz), 1.92 (s, 1H), 1.07 (br s, 9H).

### Step D: 2-((2-(((2-(3-Amino-6-methoxypyridin-2-yl)ethyl)(tert-butoxycarbonyl)amino)-methyl)-3,4-difluorophenyl)amino)-5-chloro-4-(trifluoromethyl)benzoic acid 60d

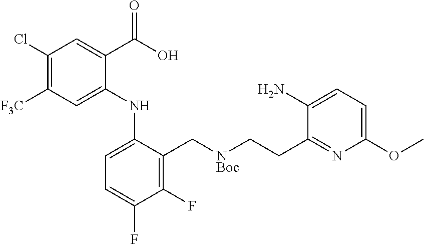 [SMILES: COc1ccc(N)c(CCN(Cc2c(Nc3cc(C(F)(F)F)c(Cl)cc3C(=O)O)ccc(F)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3,4-difluorophenyl)amino)-5-chloro-4-(trifluoromethyl)benzoic acid 60d (1.71 g, 93% yield) was prepared. HPLC/MS 1.16 min (A), [M+H]⁺ 631.2. ¹H NMR (DMSO-d₆, 400 MHz): δ 9.53 (s, 1H), 7.98 (s, 1H), 7.4-7.5 (m, 1H), 7.2-7.3 (m, 1H), 7.00 (d, 1H, J=8.8 Hz), 6.80 (s, 1H), 6.39 (d, 1H, J=8.3 Hz), 4.45 (s, 2H), 3.65 (s, 3H), 3.38 (br t, 2H, J=6.8 Hz), 2.70 (br s, 2H), 1.1-1.2 (m, 11H).

### Step E: tert-Butyl 2-chloro-8,9-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 60e

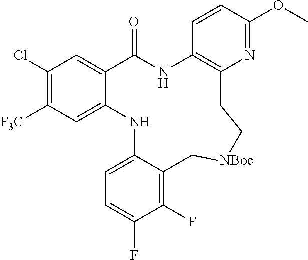 [SMILES: COc1ccc2c(n1)CCN(C(=O)OC(C)(C)C)Cc1c(ccc(F)c1F)Nc1cc(C(F)(F)F)c(Cl)cc1C(=O)N2]

Following the procedure outlined in Example 41, Step E, stirring the reaction mixture at RT for 96 h, tert-butyl 2-chloro-8,9-difluoro-15-methoxy-19-oxo-3-(trifluoromethyl)-5,10,12,13,18,19-hexahydro-11H-dibenzo[c,f]pyrido[3,2-j][1,5,9]triazacyclo-tridecine-11-carboxylate 60e (927.1 mg, 55% yield) was prepared as a white solid. HPLC/MS 1.44 min (A), [M+H]⁺ 613.2. ¹H NMR (CDCl₃, 400 MHz): δ 10.4-10.8 (m, 1H), 8.5-8.7 (m, 1H), 8.21 (s, 1H), 7.65 (s, 1H), 6.9-7.1 (m, 1H), 6.68 (s, 1H), 6.4-6.6 (m, 1H), 6.0-6.1 (m, 1H), 4.6-5.1 (m, 2H), 3.90 (s, 4H), 2.2-2.6 (m, 2H), 1.09 (s, 10H).

### Step F: tert-Butyl 2-chloro-8,9-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 60f

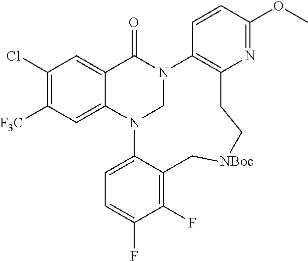 [SMILES: COc1ccc2c(n1)CCN(C(=O)OC(C)(C)C)Cc1c(ccc(F)c1F)N1CN2C(=O)c2cc(Cl)c(C(F)(F)F)cc21]

Following the procedure outlined in Example 41, Step F, stirring the reaction at 90° C. for 20 h, tert-butyl 2-chloro-8,9-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 60f (797 mg, 82% yield) was prepared as a white solid. HPLC/MS 1.42 min (A), [M+H]⁺ 625.2. ¹H NMR (CDCl₃, 400 MHz): δ 8.2-8.3 (m, 1H), 7.50 (d, 1H, J=8.8 Hz), 7.33 (d, 1H, J=8.8 Hz), 7.0-7.1 (m, 1H), 6.71 (d, 1H, J=8.3 Hz), 6.63 (s, 1H), 5.6-5.6 (m, 1H), 5.1-5.3 (m, 1H), 4.59 (d, 1H, J=11.2 Hz), 4.0-4.1 (m, 2H), 3.95 (s, 3H), 3.0-3.2 (m, 1H), 2.8-3.0 (m, 1H), 1.53 (s, 10H).

### Step G: 2-Chloro-8,9-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

### Example 60

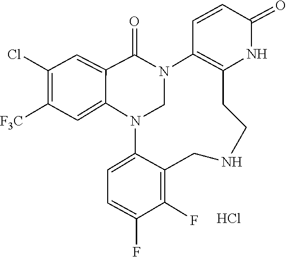 [SMILES: O=C1c2cc(Cl)c(C(F)(F)F)cc2N2CN1c1ccc(=O)[nH]c1CCNCc1c2ccc(F)c1F ; Cl]

Following the procedure outlined in Example 41, Step G, stirring the reaction mixture at 90° C. for 20.5 h, 2-chloro-8,9-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (620.4 mg, 72% yield) was prepared as a white solid. HPLC/MS 0.71 min (A), [M+H]⁺ 511.1. ¹H NMR (CD₃OD, 400 MHz): δ 8.21 (s, 1H), 7.85 (d, 1H, J=9.8 Hz), 7.7-7.8 (m, 1H), 7.51 (ddd, 1H, J=2.0, 4.3, 8.9 Hz), 6.79 (d, 1H, J=9.8 Hz), 6.68 (s, 1H), 5.63 (d, 1H, J=11.7 Hz), 5.07 (d, 1H, J=11.2 Hz), 4.4-4.6 (m, 2H), 3.48 (br d, 2H, J=5.4 Hz), 3.2-3.3 (m, 1H), 2.9-3.0 (m, 1H).

### Example 61

### 2,8-Difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

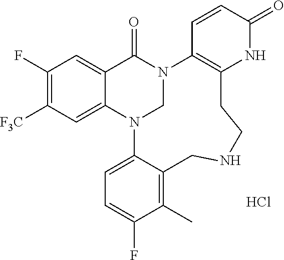 [SMILES: Cc1c(F)ccc2c1CNCCc1[nH]c(=O)ccc1N1CN2c2cc(C(F)(F)F)c(F)cc2C1=O ; Cl]

### Step A: 2,8-Difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione 61a

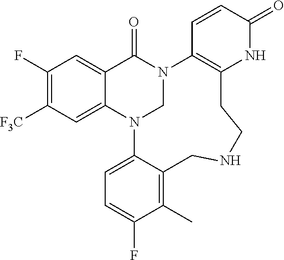 [SMILES: Cc1c(F)ccc2c1CNCCc1[nH]c(=O)ccc1N1CN2c2cc(C(F)(F)F)c(F)cc2C1=O]

To 9-chloro-2,8-difluoro-3-(trifluoromethyl)-10,11,12,13-tetrahydro-19H-5,18-methano-dibenzo[c,]pyrido[3,2-j][1,5,9]triazacyclotridecine-15,19(14H)-dione Example 58 (50.0 mg, 97.9 μmol) and (2-dicyclohexylphosphino-2′,6′-diisopropoxy-1,1′-biphenyl)[2-(2′-amino-1,1′-biphenyl)]palladium(II) methanesulfonate (4.09 mg, 4.89 μmol) in DMF (979 μL) was added dimethylzine (1 M in heptane, 41 μL, 489 μmol). The reaction mixture was heated for 3 h at 50° C., quenched with a mixture of MeCN and acetic acid, poured into a sat. solution of NaHCO₃, filtered, and washed with EtOAc. The eluent was washed with brine (3×), concentrated and purified by reverse-phase semi-prep HPLC (XSELECT CSH C18 column (150 mm×30 mm i.d. 5 μm packing diameter), eluting with 15-55% AcCN (0.1% formic acid) in water (0.1% formic acid). Product fractions were combined and evaporated to afford 2,8-difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione 61a (17 mg, 35% yield). HPLC/MS 0.71 min (A), [M+H]⁺ 491. ¹H NMR (DMSO-d₆, 400 MHz): δ 2.32 (d, J=1.47 Hz, 3H), 2.76-3.10 (m, 4H), 3.48 (br d, J=11.25 Hz, 2H), 3.87-3.96 (m, 1H), 5.05 (d, J=9.78 Hz, 1H), 5.37 (d, J=9.29 Hz, 1H), 6.22 (br d, J=9.29 Hz, 1H), 6.63 (d, J=4.89 Hz, 1H), 7.23-7.31 (m, 1H), 7.38-7.48 (m, 2H), 7.84 (d, J=10.76 Hz, 1H), 11.80-11.95 (m, 1H).

### Step B: 2,8-Difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

### Example 61

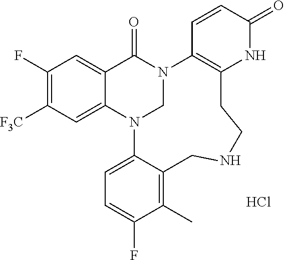 [SMILES: Cc1c(F)ccc2c1CNCCc1[nH]c(=O)ccc1N1CN2c2cc(C(F)(F)F)c(F)cc2C1=O ; Cl]

To 2,8-difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione 61a (18.0 mg, 136 μmol) in THF (10.0 mL) was added a 4M hydrogen chloride in dioxane solution (92 μL, 367 μmol). After 5 minutes the reaction was concentrated, and this process was repeated one more time. The residue was titrated with MeCN (3×1 mL), sonicated, triturated with MeCN (10 mL), then triturated with THF to afford 2,8-difluoro-9-methyl-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (8.0 mg, 38% yield). HPLC/MS 0.70 min (A), [M+H]⁺ 491.1. ¹H NMR (DMSO-d₆, 400 MHz): δ 11.70-12.21 (m, 1H), 10.14 (br d, J=1.5 Hz, 1H), 8.32-8.54 (m, 1H), 7.92 (br d, J=9.8 Hz, 1H), 7.40-7.53 (m, 3H), 6.36 (br d, J=1.0 Hz, 1H), 5.42 (br d, J=11.2 Hz, 1H), 5.02 (br d, J=11.2 Hz, 1H), 4.28 (br d, J=6.8 Hz, 1H), 4.17 (br s, 1H), 3.22-3.31 (m, 1H), 3.00-3.15 (m, 1H), 2.58-2.72 (m, 1H), 2.51 (dt, J=3.5, 1.9 Hz, 4H).

### Example 62

### 3-Bromo-2,8,9-trifluoro-10,11,12,13-tetrahydro-19H-5,18-methanodibenzo[c,f]pyrido[3,2-j][1,5,9]triazacyclotridecine-15,19(14H)-dione hydrochloride

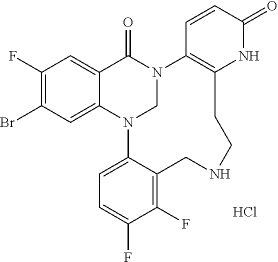 [SMILES: O=C1c2cc(F)c(Br)cc2N2CN1c1ccc(=O)[nH]c1CCNCc1c2ccc(F)c1F ; Cl]

### Step A: Methyl 4-bromo-2-((3,4-difluoro-2-formylphenyl)amino)-5-fluorobenzoate 62a

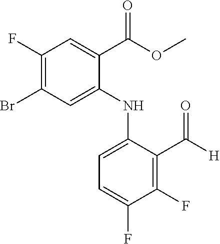 [SMILES: COC(=O)c1cc(F)c(Br)cc1Nc1ccc(F)c(F)c1C=O]

Following the preparation outlined in Example 7, Step A, substituting ethyl 2-bromo-5-(trifluoromethyl)benzoate with methyl 2-amino-4-bromo-5-fluorobenzoate, Int-3a with 2,3-difluoro-6-iodobenzaldehyde, BINAP with Xantphos, toluene with 1,4-dioxane, and stirring the reaction at 90° C. for 19 h, methyl 4-bromo-2-((3,4-difluoro-2-formylphenyl)amino)-5-fluorobenzoate 62a (2.39 g, 67% yield) was prepared as a bright yellow solid. HPLC/MS 1.32 min (B), [M+H]⁺ 385.9, 387.9. ¹H NMR (DMSO-d₆, 400 MHz): δ 10.8-10.9 (m, 1H), 10.3-10.3 (m, 1H), 7.8-7.9 (m, 2H), 7.6-7.7 (m, 1H), 7.22 (dd, 1H, J=2.7, 8.6 Hz), 3.87 (s, 3H).

### Step B: Methyl 4-bromo-2-((2-(((tert-butoxycarbonyl)(2-(6-methoxy-3-nitropyridin-2-yl)ethyl)amino)methyl)-3,4-difluorophenyl)amino)-5-fluorobenzoate 62b

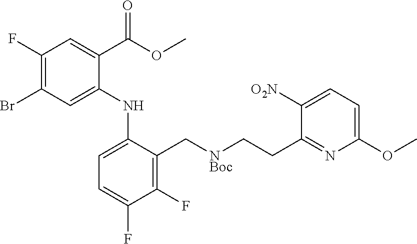 [SMILES: COC(=O)c1cc(F)c(Br)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4-bromo-2-((3,4-difluoro-2-formylphenyl)amino)-5-fluorobenzoate 62a, methyl 4-bromo-2-((2-(((tert-butoxycarbonyl)(2-(6-methoxy-3-nitropyridin-2-yl)ethyl)amino)methyl)-3,4-difluorophenyl)-amino)-5-fluorobenzoate 62b (1.55 g, 81% yield) was prepared as a light yellow solid. HPLC/MS 1.67 min (A), [M+H]⁺ 669.1, 671.1. ¹H NMR (DMSO-d₆, 400 MHz): δ 8.93 (s, 1H), 8.33 (br d, 1H, J=8.8 Hz), 7.70 (d, 1H, J=9.3 Hz), 7.4-7.5 (m, 1H), 7.1-7.2 (m, 1H), 6.87 (br d, 1H, J=6.8 Hz), 6.79 (br s, 1H), 4.50 (s, 2H), 3.8-3.9 (m, 6H), 3.55 (br s, 2H), 3.14 (t, 2H, J=6.6 Hz), 1.14 (br s, 9H).

### Step C: 4-Bromo-2-((2-(((tert-butoxycarbonyl)(2-(6-methoxy-3-nitropyridin-2-yl)ethyl-amino)methyl)-3,4-difluorophenyl)amino)-5-fluorobenzoic acid 62c

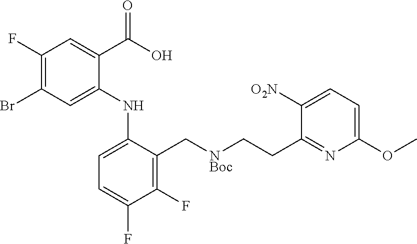 [SMILES: COc1ccc([N+](=O)[O-])c(CCN(Cc2c(Nc3cc(Br)c(F)cc3C(=O)O)ccc(F)c2F)C(=O)OC(C)(C)C)n1]

Following the procedure outlined in Example 41, Step C, stirring the reaction mixture at room temperature for 72 h, 4-bromo-2-((2-(((tert-butoxycarbonyl)(2-(6-methoxy-3-nitropyridin-2-yl)ethyl)amino)methyl)-3,4-difluorophenyl)amino)-5-fluorobenzoic acid 62c (1.53 g, 90% yield) was prepared as a light yellow solid. HPLC/MS 1.12 min (B), [M+H]⁺ 655.1, 657.1. ¹H NMR (DMSO-d₆, 400 MHz): δ 13.0-13.7 (m, 1H), 9.25 (s, 1H), 8.32 (br d, 1H, J=8.8 Hz), 7.67 (d, 1H, J=9.3 Hz), 7.4-7.5 (m, 1H), 7.1-7.2 (m, 1H), 6.86 (br s, 1H), 6.77 (br s, 1H), 4.50 (br s, 2H), 3.91 (br s, 3H), 3.57 (br t, 2H, J=6.4 Hz), 3.13 (br t, 2H, J=6.1 Hz), 1.1-1.2 (m, 9H).

### Step D: 2-((2-(2-(-(3-Amino-6-methoxypyridin-2-yl)ethyl)(tert-butoxycarbonyl)amino)-methyl)-3,4-difluorophenyl)amino)-4-bromo-5-fluorobenzoic acid 62d

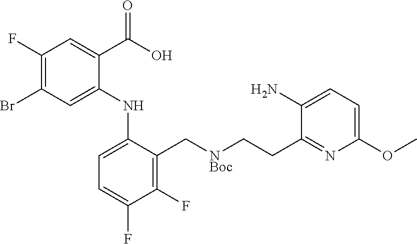 [SMILES: COc1ccc(N)c(CCN(Cc2c(Nc3cc(Br)c(F)cc3C(=O)O)ccc(F)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3,4-difluorophenyl)amino)-4-bromo-5-fluorobenzoic acid 62d (1.33 g, 84% yield) as a light brown solid. HPLC/MS 1.01 min (B), [M+H]⁺ 625.0, 627.0. ¹H NMR (DMSO-d₆, 400 MHz): δ 7.65 (br d, J=9.8 Hz, 1H), 7.23-7.39 (m, 1H), 7.05 (br dd, J=9.0, 5.1 Hz, 1H), 6.90-6.98 (m, 1H), 6.69 (br d, J=5.9 Hz, 1H), 6.26-6.36 (m, 1H), 4.47 (br s, 2H), 3.61 (s, 2H), 3.27-3.41 (m, 4H), 2.62 (br t, J=7.1 Hz, 1H), 1.21 (s, 9H).

### Step E: tert-Butyl 3-bromo-2,8,9-trifluoro-15-methoxy-19-oxo-5,10,12,13,18,19-hexa-hydro-11H-dibenzo[c,f]pyrido[3,2-j][1,5,9]triazacyclotridecine-11-carboxylate 62e

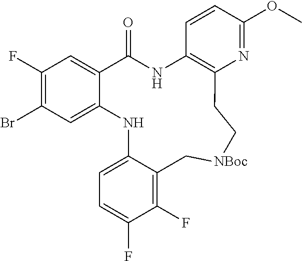 [SMILES: COc1ccc2c(n1)CCN(C(=O)OC(C)(C)C)Cc1c(ccc(F)c1F)Nc1cc(Br)c(F)cc1C(=O)N2]

Following the procedure outlined in Example 41, Step E, substituting HATU with pyoxim, and stirring the reaction mixture at room temperature for 1 h, tert-butyl 3-bromo-2,8,9-trifluoro-15-methoxy-19-oxo-5,10,12,13,18,19-hexahydro-11H-dibenzo[c,f]pyrido[3,2-j][1,5,9]triaza-cyclotridecine-11-carboxylate 62e (238 mg, 27% yield) was prepared as a light pink solid. HPLC/MS 1.45 min (B), [M+H]⁺ 607.0, 609.0. ¹H NMR (DMSO-d₆, 400 MHz): δ 10.22 (br s, 1H), 7.86 (br d, 1H, J=8.3 Hz), 7.77 (br d, 1H, J=8.3 Hz), 7.71 (s, 1H), 7.60 (br s, 1H), 7.15 (q, 1H, J=9.3 Hz), 6.69 (d, 1H, J=8.8 Hz), 6.16 (br s, 1H), 4.77 (s, 2H), 3.80 (s, 3H), 3.17 (br s, 2H), 2.73 (br s, 2H), 1.41 (br s, 9H).

### Step F: tert-Butyl 3-bromo-2,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 62f

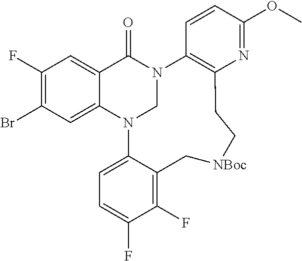 [SMILES: COc1ccc2c(n1)CCN(C(=O)OC(C)(C)C)Cc1c(ccc(F)c1F)N1CN2C(=O)c2cc(F)c(Br)cc21]

Following the procedure outlined in Example 41, Step F, stirring the reaction at 90° C. for 65 h, tert-butyl 3-bromo-2,8,9-trifluoro-15-methoxy-19-oxo-12,13-dihydro-19H-5,18-methano-dibenzo[c,f]pyrido[3,2-j][1,5,9]triazacyclotridecine-11(10H)-carboxylate 62f (392 mg, 79% yield) was prepared as a light yellow solid. HPLC/MS 1.44 min (B), [M+H]⁺ 619.1, 621.1. ¹H NMR (DMSO-d₆, 400 MHz): δ 7.7-7.8 (m, 2H), 7.6-7.6 (m, 1H), 7.32 (ddd, 1H, J=2.2, 4.4, 6.6 Hz), 6.8-6.8 (m, 1H), 6.63 (d, 1H, J=5.4 Hz), 5.58 (d, 1H, J=11.7 Hz), 5.01 (br d, 1H, J=14.2 Hz), 4.79 (br d, 1H, J=10.8 Hz), 3.8-3.9 (m, 4H), 3.66 (br d, 1H, J=6.4 Hz), 2.93 (br t, 1H, J=10.0 Hz), 2.5-2.6 (m, 1H), 1.41 (s, 9H).

### Step G: 3-Bromo-2,8,9-trifluoro-10,11,12,13-tetrahydro-19H-5,18-methanodibenzo-[c,f]pyrido[3,2-j][1,5,9]triazacyclotridecine-15,19(14H)-dione hydrochloride Example 62

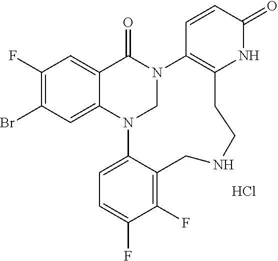 [SMILES: O=C1c2cc(F)c(Br)cc2N2CN1c1ccc(=O)[nH]c1CCNCc1c2ccc(F)c1F ; Cl]

Following the procedure outlined in Example 41, Step G, stirring the reaction mixture at 80° C. for 19 h, 3-bromo-2,8,9-trifluoro-10,11,12,13-tetrahydro-19H-5,18-methano-dibenzo-[c,f]pyrido[3,2-j][1,5,9]triazacyclotridecine-15,19(14H)-dione hydrochloride (38 mg, 35% yield) was prepared as a white solid. HPLC/MS 0.99 min (B), [M+H]⁺ 505.0, 507.0. ¹H NMR (DMSO-d₆, 400 MHz): δ 10.22 (br s, 1H), 9.02 (br s, 1H), 7.7-7.8 (m, 2H), 7.4-7.5 (m, 2H), 6.55 (d, 1H, J=5.9 Hz), 6.33 (d, 1H, J=9.3 Hz), 5.42 (d, 2H, J=11.2 Hz), 4.96 (d, 1H, J=11.7 Hz), 4.2-4.3 (m, 1H), 4.1-4.2 (m, 1H), 3.2-3.3 (m, 1H), 3.0-3.1 (m, 2H), 2.6-2.7 (m, 1H).

### Example 63

### 3-Bromo-2,8-difluoro-10,11,12,13-tetrahydro-19H-5,18-methanodibenzo[c,f]pyrido[3,2-j][1,5,9]triazacyclotridecine-15,19(14H)-dione hydrochloride

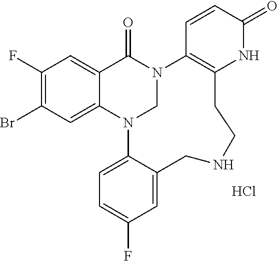 [SMILES: O=C1c2cc(F)c(Br)cc2N2CN1c1ccc(=O)[nH]c1CCNCc1cc(F)ccc12 ; Cl]

### Step A: Methyl 4-bromo-5-fluoro-2-((4-fluoro-2-formylphenyl)amino)benzoate 63a

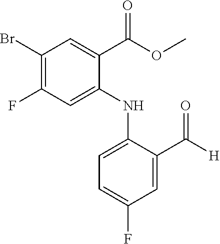 [SMILES: COC(=O)c1cc(Br)c(F)cc1Nc1ccc(F)cc1C=O]

Following the preparation outlined in Example 7, Step A, substituting ethyl 2-bromo-5-(trifluoromethyl)benzoate with methyl 2-amino-4-bromo-5-fluorobenzoate, Int-3a with 2-bromo-5-fluorobenzaldehyde, BINAP with Xantphos, toluene with 1,4-dioxane, and stirring the reaction at 90° C. for 19 h, methyl 4-bromo-5-fluoro-2-((4-fluoro-2-formylphenyl)amino)benzoate 63a (3.30 g, 39% yield) was prepared as a light yellow solid. HPLC/MS 1.29 min (B), [M+H]⁺ 367.9, 369.9. ¹H NMR (DMSO-d₆, 400 MHz): δ 10.63 (s, 1H), 9.96 (s, 1H), 7.81 (d, 1H, J=9.3 Hz), 7.7-7.8 (m, 1H), 7.5-7.5 (m, 2H), 3.87 (s, 3H), 1.2-1.2 (m, 1H).

### Step B: Methyl 4-bromo-2-((2-(((tert-butoxycarbonyl)(2-(6-methoxy-3-nitropyridin-2-yl)ethyl)amino)methyl)-4-fluorophenyl)amino)-5-fluorobenzoate 63b

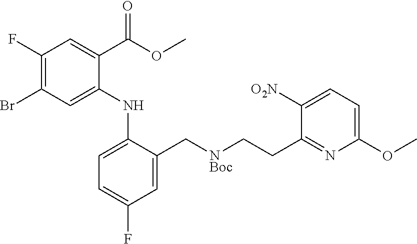 [SMILES: COC(=O)c1cc(F)c(Br)cc1Nc1ccc(F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4-bromo-2-((3,4-difluoro-2-formylphenyl)amino)-5-fluorobenzoate 63a, 4-bromo-2-((2-(((tert-butoxycarbonyl)(2-(6-methoxy-3-nitropyridin-2-yl)ethyl)amino)methyl)-4-fluorophenyl)amino)-5-fluorobenzoate 63b (3.95 g, 70% yield) was prepared as a yellow solid. HPLC/MS 1.64 min (B), [M+H]⁺ 651.0, 653.0. ¹H NMR (DMSO-d₆, 400 MHz): δ 8.8-8.9 (m, 1H), 8.30 (d, 1H, J=8.8 Hz), 7.72 (d, 1H, J=9.3 Hz), 7.33 (t, 1H, J=6.6 Hz), 7.2-7.2 (m, 1H), 7.09 (br s, 1H), 6.84 (br s, 1H), 6.5-6.7 (m, 1H), 4.31 (br s, 2H), 3.88 (s, 3H), 3.86 (s, 3H), 3.62 (br s, 2H), 3.15 (br s, 2H), 1.20 (s, 9H).

### Step C: 4-Bromo-2-((2-(((tert-butoxycarbonyl)(2-(6-methoxy-3-nitropyridin-2-yl)ethyl)-amino)methyl)-4-fluorophenyl)amino)-5-fluorobenzoic acid 63c

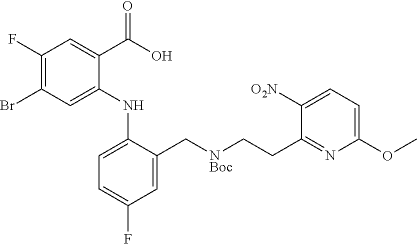 [SMILES: COc1ccc([N+](=O)[O-])c(CCN(Cc2cc(F)ccc2Nc2cc(Br)c(F)cc2C(=O)O)C(=O)OC(C)(C)C)n1]

Following the procedure outlined in Example 41, Step C, stirring the reaction mixture at 50° C. for 20 h, 4-bromo-2-((2-(((tert-butoxycarbonyl)(2-(6-methoxy-3-nitropyridin-2-yl)ethyl)-amino)methyl)-4-fluorophenyl)amino)-5-fluorobenzoic acid 63c (1.53 g, 98% yield) was prepared as a light yellow solid. HPLC/MS 1.08 min (B), [M+H]⁺ 637.0, 638.9. ¹H NMR (DMSO-d₆, 400 MHz): δ 13.1-13.8 (m, 1H), 9.12 (s, 1H), 8.30 (br d, 1H, J=9.3 Hz), 7.70 (d, 1H, J=9.3 Hz), 7.35 (dd, 1H, J=5.4, 8.3 Hz), 7.2-7.2 (m, 1H), 7.0-7.1 (m, 1H), 6.8-6.9 (m, 1H), 6.5-6.7 (m, 1H), 4.3-4.4 (m, 2H), 3.86 (s, 3H), 3.6-3.7 (m, 2H), 3.1-3.2 (m, 2H), 1.2-1.2 (m, 9H).

### Step D: 2-((2-(((2-(3-Amino-6-methoxypyridin-2-yl)ethyl)(tert-butoxycarbonyl)amino)-methyl)-4-fluorophenyl)amino)-4-bromo-5-fluorobenzoic acid 63d

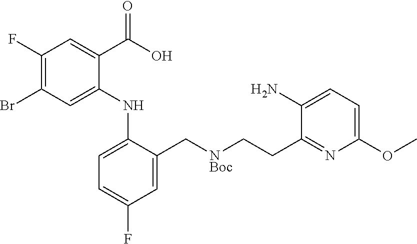 [SMILES: COc1ccc(N)c(CCN(Cc2cc(F)ccc2Nc2cc(Br)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-fluorophenyl)amino)-4-bromo-5-fluorobenzoic acid 63d (3.56 g, 83% yield) as a yellow solid. HPLC/MS 0.99 min (B), [M+H]⁺ 607.0, 609.0. ¹H NMR (DMSO-d₆, 400 MHz): δ 7.69 (br d, J=9.8 Hz, 1H), 7.20-7.30 (m, 1H), 7.07 (td, J=8.4, 2.7 Hz, 1H), 6.96 (d, J=8.8 Hz, 1H), 6.89 (br d, J=9.8 Hz, 1H), 6.55-6.74 (m, 1H), 6.34 (d, J=8.3 Hz, 1H), 4.34 (br s, 2H), 3.56-3.64 (m, 3H), 3.48 (br s, 2H), 2.71 (br t, J=7.3 Hz, 2H), 1.32 (br s, 9H).

### Step E: tert-Butyl 3-bromo-2,8-difluoro-15-methoxy-19-oxo-5,10,12,13,18,19-hexahydro-11H-dibenzo[c,f]pyrido[3,2-j][1,5,9]triazacyclotridecine-11-carboxylate 63e

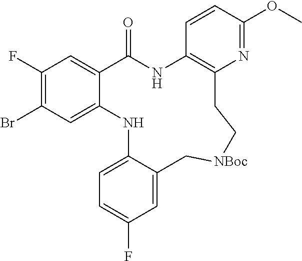 [SMILES: COc1ccc2c(n1)CCN(C(=O)OC(C)(C)C)Cc1cc(F)ccc1Nc1cc(Br)c(F)cc1C(=O)N2]

Following the procedure outlined in Example 41, Step E, substituting HATU with pyoxim, and stirring the reaction mixture at RT for 30 min, tert-butyl 3-bromo-2,8-difluoro-15-methoxy-19-oxo-5,10,12,13,18,19-hexahydro-11H-dibenzo[c,f]pyrido[3,2-j][1,5,9]triaza-cyclotridecine-11-carboxylate 63e (856 mg, 35% yield) was prepared as a light pink solid. HPLC/MS 1.43 min (B), [M+H]⁺ 589.0, 591.0. ¹H NMR (DMSO-d₆, 400 MHz): δ 10.2-10.6 (m, 1H), 7.87 (d, 1H, J=8.8 Hz), 7.77 (br s, 1H), 7.4-7.5 (m, 2H), 7.19 (br s, 1H), 6.99 (dt, 1H, J=2.9, 8.6 Hz), 6.7-6.7 (m, 1H), 6.4-6.6 (m, 1H), 4.59 (s, 2H), 3.80 (s, 3H), 3.3-3.4 (m, 2H), 1.2-1.5 (m, 11H).

### Step F: tert-Butyl 3-bromo-2,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 63f

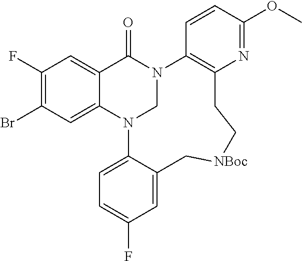 [SMILES: COc1ccc2c(n1)CCN(C(=O)OC(C)(C)C)Cc1cc(F)ccc1N1CN2C(=O)c2cc(F)c(Br)cc21]

Following the procedure outlined in Example 41, Step F, stirring the reaction at 90° C. for 19 h, tert-butyl 3-bromo-2,8-difluoro-15-methoxy-19-oxo-12,13-dihydro-19H-5,18-methano-dibenzo[c,f]pyrido[3,2-j][1,5,9]triazacyclotridecine-11(10H)-carboxylate 63f (724 mg, 82% yield) was prepared as a light yellow solid. HPLC/MS 1.46 min (B), [M+H]⁺ 601.0, 603.0. ¹H NMR (DMSO-d₆, 400 MHz): δ 7.75 (d, 1H, J=8.8 Hz), 7.70 (d, 1H, J=8.3 Hz), 7.49 (dd, 1H, J=5.4, 8.8 Hz), 7.3-7.4 (m, 2H), 6.78 (d, 1H, J=8.3 Hz), 6.46 (d, 1H, J=5.9 Hz), 5.58 (d, 1H, J=11.2 Hz), 4.83 (d, 1H, J=11.7 Hz), 4.71 (d, 1H, J=14.7 Hz), 3.9-4.0 (m, 1H), 3.85 (s, 3H), 3.6-3.8 (m, 1H), 2.9-3.0 (m, 1H), 2.6-2.7 (m, 1H), 2.5-2.6 (m, 1H), 1.45 (s, 9H).

### Step G: 3-Bromo-2,8-difluoro-10,11,12,13-tetrahydro-19H-5,18-methanodibenzo-[c,f]pyrido[3,2-j][1,5,9]triazacyclotridecine-15,19(14H)-dione hydrochloride

### Example 63

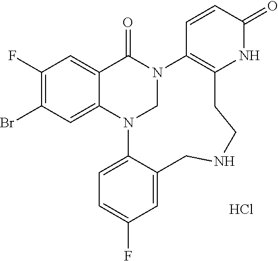 [SMILES: O=C1c2cc(F)c(Br)cc2N2CN1c1ccc(=O)[nH]c1CCNCc1cc(F)ccc12 ; Cl]

Following the procedure outlined in Example 41, Step G, stirring the reaction mixture at 90° C. for 17 h, 3-bromo-2,8-difluoro-10,11,12,13-tetrahydro-19H-5,18-methanodibenzo-[c,f]pyrido[3,2-j][1,5,9]triazacyclotridecine-15,19(14H)-dione hydrochloride (535 mg, 84% yield) was prepared as a white solid. HPLC/MS 0.97 min (B), [M+H]⁺ 487.0, 489.0. ¹H NMR (DMSO-d₆, 400 MHz): δ 10.52 (br s, 1H), 8.92 (br s, 1H), 7.7-7.8 (m, 2H), 7.61 (dd, 1H, J=5.6, 8.6 Hz), 7.50 (dd, 1H, J=3.4, 8.3 Hz), 7.44 (d, 1H, J=9.8 Hz), 6.47 (d, 1H, J=5.4 Hz), 6.33 (d, 1H, J=9.8 Hz), 5.44 (d, 1H, J=11.7 Hz), 4.90 (d, 2H, J=11.7 Hz), 4.0-4.2 (m, 2H), 3.1-3.2 (m, 1H), 3.0-3.1 (m, 2H), 2.6-2.7 (m, 1H).

### Example 64

### 9-Chloro-2,3,8-trifluoro-10,11,12,13-tetrahydro-19H-5,18-methanodibenzo[c,f]pyrido[3,2-i][1,5,9]triazacyclotridecine-15,19(14H)-dione hydrochloride

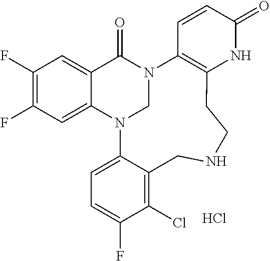 [SMILES: O=C1c2cc(F)c(F)cc2N2CN1c1ccc(=O)[nH]c1CCNCc1c2ccc(F)c1Cl ; Cl]

### Step A: Methyl 2-((3-chloro-4-fluoro-2-formylphenyl)amino)-4,5-difluorobenzoate 64a

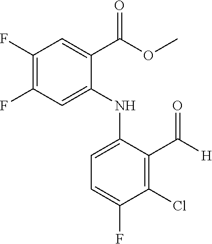 [SMILES: COC(=O)c1cc(F)c(F)cc1Nc1ccc(F)c(Cl)c1C=O]

Following the preparation outlined in Example 7, Step A, substituting ethyl 2-bromo-5-(trifluoromethyl)benzoate with methyl 2-amino-4,5-difluorobenzoate, Int-3a with 6-bromo-2-chloro-3-fluorobenzaldehyde, BINAP with Xantphos, toluene with 1,4-dioxane, and stirring the reaction at 80° C. for 20 h, methyl 2-((3-chloro-4-fluoro-2-formylphenyl)amino)-4,5-difluorobenzoate 64a (2.59 g, 67% yield) was prepared as a yellow solid. HPLC/MS 1.26 min (A), [M+H]⁺ 343.9. ¹H NMR (CDCl₃, 400 MHz): δ 11.4-11.7 (m, 1H), 10.60 (d, 1H, J=1.0 Hz), 7.8-8.0 (m, 1H), 7.4-7.4 (m, 1H), 7.3-7.3 (m, 1H), 7.3-7.3 (m, 1H), 7.3-7.3 (m, 1H), 4.00 (s, 3H).

### Step B: Methyl 2-((2-(((tert-butoxycarbonyl)(2-(6-methoxy-3-nitropyridin-2-yl)ethyl-amino)methyl)-3-chloro-4-fluorophenyl)amino)-4,5-difluorobenzoate 64b

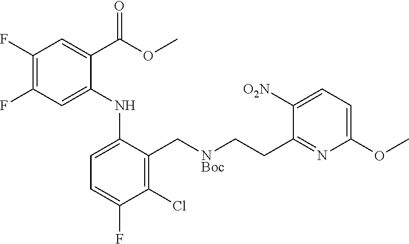 [SMILES: COC(=O)c1cc(F)c(F)cc1Nc1ccc(F)c(Cl)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3-chloro-4-fluoro-2-formylphenyl)amino)-4,5-difluorobenzoate 64a, methyl 2-((2-(((tert-butoxycarbonyl)(2-(6-methoxy-3-nitropyridin-2-yl)ethyl)amino)methyl)-3-chloro-4-fluorophenyl)amino)-4,5-difluorobenzoate 64b (3.87 g, 80% yield) was prepared as a white foam. HPLC/MS 1.55 min (A), [M+H]⁺ 625.2. ¹H NMR (CDCl₃, 400 MHz): δ 9.2-9.4 (m, 1H), 8.23 (d, 1H, J=9.3 Hz), 7.7-7.9 (m, 1H), 7.1-7.3 (m, 2H), 6.6-6.8 (m, 1H), 6.4-6.5 (m, 1H), 4.7-4.9 (m, 2H), 3.98 (s, 3H), 3.92 (s, 3H), 3.6-3.7 (m, 2H), 3.29 (s, 2H), 1.35 (s, 9H).

### Step C: 2-((2-(((tert-Butoxycarbonyl)(2-(6-methoxy-3-nitropyridin-2-yl)ethyl)amino)-methyl)-3-chloro-4-fluorophenyl)amino)-4,5-difluorobenzoic acid 64c

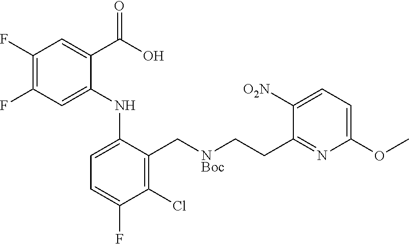 [SMILES: COc1ccc([N+](=O)[O-])c(CCN(Cc2c(Nc3cc(F)c(F)cc3C(=O)O)ccc(F)c2Cl)C(=O)OC(C)(C)C)n1]

Following the procedure outlined in Example 41, Step C, stirring the reaction mixture at 50° C. for 7 h, then at 23° C. for 96 h, 2-((2-(((tert-butoxycarbonyl)(2-(6-methoxy-3-nitropyridin-2-yl)ethyl)amino)methyl)-3-chloro-4-fluorophenyl)amino)-4,5-difluorobenzoic acid 64c (3.96 g, 100% yield) was prepared as an off white foam. HPLC/MS 1.44 min (A), [M+H]⁺ 611.1. ¹H NMR (CDCl₃, 400 MHz): δ 9.2-9.4 (m, 1H), 8.24 (d, 1H, J=8.8 Hz), 7.7-7.9 (m, 1H), 7.2-7.3 (m, 2H), 6.6-6.8 (m, 1H), 6.4-6.5 (m, 1H), 4.76 (s, 2H), 3.99 (s, 3H), 3.6-3.8 (m, 2H), 3.2-3.4 (m, 2H), 1.2-1.4 (m, 10H).

### Step D: 2-((2-(((2-(3-Amino-6-methoxypyridin-2-yl)ethyl)(tert-butoxycarbonyl)amino)-methyl)-3-chloro-4-fluorophenyl)amino)-4,5-difluorobenzoic acid 64d

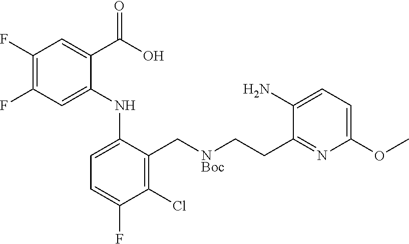 [SMILES: COc1ccc(N)c(CCN(Cc2c(Nc3cc(F)c(F)cc3C(=O)O)ccc(F)c2Cl)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3-chloro-4-fluorophenyl)-amino)-4,5-difluorobenzoic acid 64d (1.71 g, 93% yield) was prepared. HPLC/MS 1.11 min (A), [M+H]⁺ 581.1. ¹H NMR (DMSO-d₆, 400 MHz): δ 9.43 (s, 1H), 7.7-7.9 (m, 1H), 7.42 (s, 1H), 7.35 (br d, 1H, J=5.4 Hz), 6.97 (d, 1H, J=8.3 Hz), 6.43 (br s, 1H), 6.38 (d, 1H, J=8.3 Hz), 4.54 (s, 2H), 3.64 (s, 3H), 3.36 (br t, 2H, J=7.3 Hz), 2.6-2.8 (m, 2H), 1.1-1.3 (m, 11H).

### Step E: tert-Butyl 9-chloro-2,3,8-trifluoro-15-methoxy-19-oxo-5,10,12,13,18,19-hexa-hydro-11H-dibenzo[c,f]pyrido[3,2-j][1,5,9]triazacyclotridecine-11-carboxylate 64e

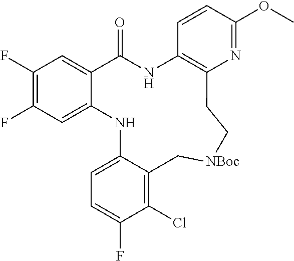 [SMILES: COc1ccc2c(n1)CCN(C(=O)OC(C)(C)C)Cc1c(ccc(F)c1Cl)Nc1cc(F)c(F)cc1C(=O)N2]

Following the procedure outlined in Example 41, Step E, stirring the reaction mixture at room temperature for 19 h, tert-butyl 9-chloro-2,3,8-trifluoro-15-methoxy-19-oxo-5,10,12,13,18,19-hexahydro-11H-dibenzo[c,f]pyrido[3,2-j][1,5,9]triazacyclotridecine-11-carboxylate 64e (927.1 mg, 55% yield) was prepared as a white solid. HPLC/MS 1.42 min (B), [M+H]⁺ 563.2. ¹H NMR (CDCl₃, 400 MHz): δ 10.7-11.1 (m, 1H), 8.3-8.4 (m, 1H), 8.29 (d, 1H, J=8.8 Hz), 7.1-7.2 (m, 1H), 7.0-7.1 (m, 1H), 6.65 (d, 1H, J=8.8 Hz), 6.4-6.5 (m, 1H), 6.2-6.3 (m, 1H), 4.6-5.3 (m, 2H), 4.0-4.5 (m, 1H), 3.89 (s, 3H), 3.2-3.7 (m, 1H), 1.9-2.7 (m, 2H), 1.08 (br s, 9H).

### Step F: tert-Butyl 9-chloro-2,3,8-trifluoro-15-methoxy-19-oxo-12,13-dihydro-19H-5,18-methanodibenzo[c,f]pyrido[3,2-j][1,5,9]triazacyclotridecine-11(10H)-carboxylate 64f

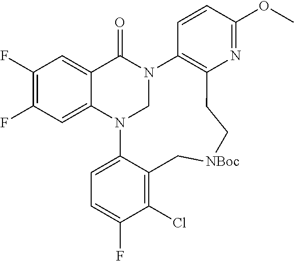 [SMILES: COc1ccc2c(n1)CCN(C(=O)OC(C)(C)C)Cc1c(ccc(F)c1Cl)N1CN2C(=O)c2cc(F)c(F)cc21]

Following the procedure outlined in Example 41, Step F, stirring the reaction at 90° C. for 24 h, tert-butyl 9-chloro-2,3,8-trifluoro-15-methoxy-19-oxo-12,13-dihydro-19H-5,18-methano-dibenzo[c,f]pyrido[3,2-j][1,5,9]triazacyclotridecine-11(10H)-carboxylate 64f (1.81 g, 75% yield) was prepared as an off white solid. HPLC/MS 1.39 min (A), [M+H]⁺ 575.1. ¹H NMR (CDCl₃, 400 MHz): δ 7.96 (dd, 1H, J=8.6, 10.0 Hz), 7.49 (d, 1H, J=8.3 Hz), 7.3-7.3 (m, 1H), 7.15 (s, 1H), 6.68 (d, 1H, J=8.8 Hz), 6.0-6.3 (m, 1H), 5.61 (d, 1H, J=10.8 Hz), 5.0-5.4 (m, 1H), 4.52 (d, 1H, J=11.2 Hz), 4.16 (br d, 2H, J=6.8 Hz), 3.94 (s, 4H), 2.7-3.2 (m, 2H), 2.2-2.6 (m, 1H), 1.54 (s, 9H).

### Step G: 9-Chloro-2,3,8-trifluoro-10,11,12,13-tetrahydro-19H-5,18-methanodibenzo-[c,f]pyrido[3,2-j][1,5,9]triazacyclotridecine-15,19(14H)-dione hydrochloride

### Example 64

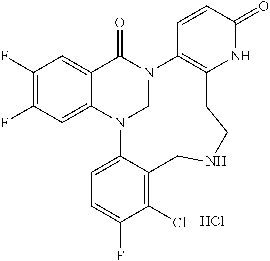 [SMILES: O=C1c2cc(F)c(F)cc2N2CN1c1ccc(=O)[nH]c1CCNCc1c2ccc(F)c1Cl ; Cl]

Following the procedure outlined in Example 41, Step G, stirring the reaction mixture at 90° C. for 74 h, 9-chloro-2,3,8-trifluoro-10,11,12,13-tetrahydro-19H-5,18-methanodibenzo-[c,f]pyrido[3,2-j][1,5,9]triazacyclotridecine-15,19(14H)-dione hydrochloride (1.64 g, 100% yield) was prepared as a white solid. HPLC/MS 0.59 min (A), [M+H]⁺ 461.1. ¹H NMR (CD₃OD, 400 MHz): δ 7.90 (dd, 1H, J=8.8, 10.3 Hz), 7.6-7.7 (m, 2H), 7.6-7.6 (m, 1H), 6.62 (d, 1H, J=9.3 Hz), 6.26 (dd, 1H, J=6.4, 11.2 Hz), 5.59 (d, 1H, J=11.7 Hz), 4.96 (d, 1H, J=11.2 Hz), 4.52 (d, 2H, J=2.9 Hz), 3.52 (br d, 1H, J=7.3 Hz), 3.3-3.4 (m, 2H), 3.17 (br d, 1H, J=1.5 Hz), 2.9-3.0 (m, 1H).

### Example 65

### 9-Chloro-2,3,8-trifluoro-10,11,12,13-tetrahydro-19H-5,18-methanodibenzo[c,f]pyrido[3,2-j][1,5,9]triazacyclotridecine-15,19(14H)-dione-20,20-d₂ hydrochloride

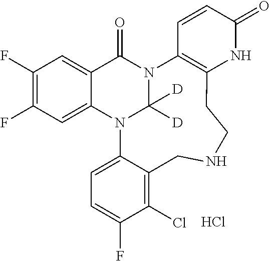 [SMILES: [2H]C1([2H])N2C(=O)c3cc(F)c(F)cc3N1c1ccc(F)c(Cl)c1CNCCc1[nH]c(=O)ccc12 ; Cl]

### Step A: tert-Butyl 9-chloro-2,3,8-trifluoro-15-methoxy-19-oxo-12,13-dihydro-19H-5,18-methanodibenzo[c,f]pyrido[3,2-j][1,5,9]triazacyclotridecine-11(10H)-carboxylate-20,20-d₂ 65a

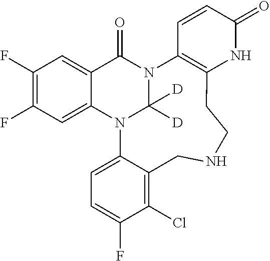 [SMILES: [2H]C1([2H])N2C(=O)c3cc(F)c(F)cc3N1c1ccc(F)c(Cl)c1CNCCc1[nH]c(=O)ccc12]

Following the procedure outlined in Example 65, Step F, substituting CH₂I₂ with CD₂I₂ and stirring the reaction at 80° C. for 30 h, tert-butyl 9-chloro-2,3,8-trifluoro-15-methoxy-19-oxo-12,13-dihydro-19H-5,18-methanodibenzo[c,f]pyrido[3,2-j][1,5,9]triazacyclotridecine-11(10H)-carboxylate-20,20-d₂ 65b (384 mg, 38% yield) was prepared. HPLC/MS 1.39 min (A), [M+H]⁺ 577. ¹H NMR (DMSO-d₆, 400 MHz): δ 7.84 (dd, 1H, J=8.8, 10.8 Hz), 7.69 (d, 1H, J=8.3 Hz), 7.6-7.6 (m, 1H), 7.4-7.5 (m, 1H), 6.78 (d, 1H, J=8.8 Hz), 6.45 (dd, 1H, J=6.4, 11.7 Hz), 5.6-5.6 (m, 1H), 4.8-5.0 (m, 1H), 3.9-4.2 (m, 1H), 3.85 (s, 3H), 3.5-3.8 (m, 1H), 2.88 (br dd, 1H, J=3.7, 8.1 Hz), 2.59 (br dd, 1H, J=7.3, 11.2 Hz), 1.44 (br s, 9H).

### Step B: 9-Chloro-2,3,8-trifluoro-10,11,12,13-tetrahydro-19H-5,18-methanodibenzo-[c,f]pyrido[3,2-j][1,5,9]triazacyclotridecine-15,19(14H)-dione-20,20-d₂ hydrochloride

### Example 65

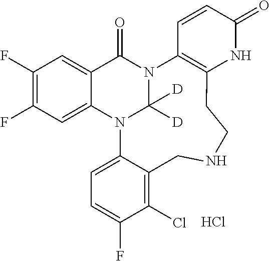 [SMILES: [2H]C1([2H])N2C(=O)c3cc(F)c(F)cc3N1c1ccc(F)c(Cl)c1CNCCc1[nH]c(=O)ccc12 ; Cl]

Following the procedure outlined in Example 41, Step G, stirring the reaction mixture at 85° C. for 5 days, 9-chloro-2,3,8-trifluoro-10,11,12,13-tetrahydro-19H-5,18-methanodibenzo-[c,f]pyrido[3,2-j][1,5,9]triazacyclotridecine-15,19(14H)-dione-20,20-d₂ hydrochloride (285 g, 90% yield) was prepared as a white solid. HPLC/MS 0.56 min (A), [M+H]⁺ 463. ¹H NMR (CD₃OD, 400 MHz): δ 7.92 (dd, 1H, J=8.3, 10.3 Hz), 7.80 (br d, 1H, J=9.3 Hz), 7.6-7.7 (m, 1H), 7.6-7.6 (m, 1H), 6.74 (br d, 1H, J=9.8 Hz), 6.30 (dd, 1H, J=6.4, 11.2 Hz), 4.9-5.6 (m, 1H), 4.5-4.6 (m, 1H), 4.5-4.5 (m, 1H), 3.4-3.6 (m, 3H), 3.2-3.3 (m, 1H), 2.9-3.0 (m, 1H).

### Example 66

### 8-Chloro-2-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

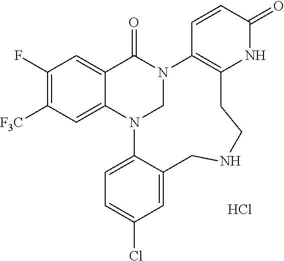 [SMILES: O=C1c2cc(F)c(C(F)(F)F)cc2N2CN1c1ccc(=O)[nH]c1CCNCc1cc(Cl)ccc12 ; Cl]

### Step A: Methyl 2-((4-chloro-2-formylphenyl)amino)-5-fluoro-4-(trifluoromethyl)-benzoate 66a

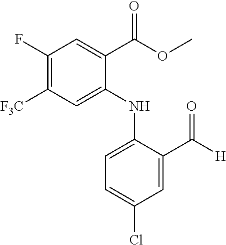 [SMILES: COC(=O)c1cc(F)c(C(F)(F)F)cc1Nc1ccc(Cl)cc1C=O]

Following the preparation outlined in Example 7, Step A, substituting ethyl 2-bromo-5-(trifluoromethyl)benzoate with methyl 2-amino-5-fluoro-4-(trifluoromethyl)benzoate, Int-3a with 2-bromo-5-chlorobenzaldehyde, BINAP with Xantphos, toluene with 1,4-dioxane, and stirring the reaction at 90° C. for 22 h, methyl 2-((4-chloro-2-formylphenyl)amino)-5-fluoro-4-(trifluoro-methyl)benzoate 66a (2.50 g, 29% yield) was prepared as a yellow solid. HPLC/MS 1.33 min (A), [M+H]⁺ 376.0. ¹H NMR (DMSO-d₆, 400 MHz): δ 10.88 (s, 1H), 9.97 (s, 1H), 7.98 (d, J=2.4 Hz, 1H), 7.95 (d, J=10.8 Hz, 1H), 7.86 (d, J=5.9 Hz, 1H), 7.59 (dd, J=9.0, 2.7 Hz, 1H), 7.47 (d, J=9.3 Hz, 1H), 3.90 (s, 3H).

### Step B: Methyl 2-((2-(((tert-butoxycarbonyl)(2-(6-methoxy-3-nitropyridin-2-yl)ethyl-amino)methyl)-4-chlorophenyl)amino)-5-fluoro-4-(trifluoromethyl)benzoate 66b

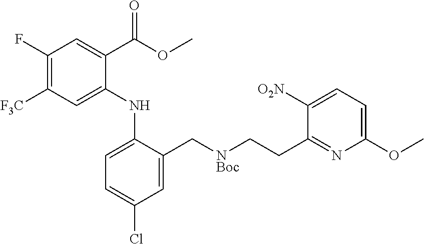 [SMILES: COC(=O)c1cc(F)c(C(F)(F)F)cc1Nc1ccc(Cl)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((4-chloro-2-formylphenyl)amino)-5-fluoro-4-(trifluoromethyl)benzoate 66a, methyl 2-((2-(((tert-butoxycarbonyl)(2-(6-methoxy-3-nitropyridin-2-yl)ethyl)amino)methyl)-4-chlorophenyl)amino)-5-fluoro-4-(trifluoromethyl)benzoate 66b (3.72 g, 85% yield) was prepared as a yellow foam. HPLC/MS 1.70 min (A), [M+H]⁺ 657.4. ¹H NMR (DMSO-d₆, 400 MHz): δ 8.93 (s, 1H), 8.31 (d, J=8.8 Hz, 1H), 7.86 (d, J=11.2 Hz, 1H), 7.39-7.45 (m, 1H), 7.33-7.37 (m, 1H), 7.20-7.33 (m, 1H), 6.85 (br d, J=8.8 Hz, 1H), 4.34 (br d, J=1.5 Hz, 2H), 3.90 (s, 3H), 3.87 (s, 3H), 3.61 (br s, 2H), 3.16 (br s, 2H), 1.19 (s, 9H).

### Step C: 2-((2-(((tert-Butoxycarbonyl)(2-(6-methoxy-3-nitropyridin-2-yl)ethyl)amino)-methyl)-4-chlorophenyl)amino)-5-fluoro-4-(trifluoromethyl)benzoic acid hydrochloride 66c

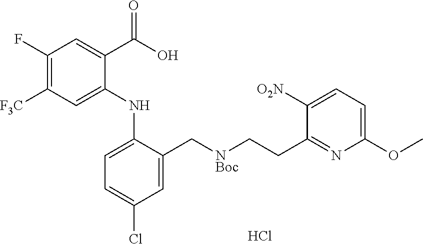 [SMILES: COc1ccc([N+](=O)[O-])c(CCN(Cc2cc(Cl)ccc2Nc2cc(C(F)(F)F)c(F)cc2C(=O)O)C(=O)OC(C)(C)C)n1 ; Cl]

Following the procedure outlined in Example 41, Step C, stirring the reaction mixture at ambient temperature for 6 h, 2-((2-(((tert-butoxycarbonyl)(2-(6-methoxy-3-nitropyridin-2-yl)ethyl)amino)methyl)-4-chlorophenyl)amino)-5-fluoro-4-(trifluoromethyl)benzoic acid hydrochloride 66c (3.68 g, 96% yield) was prepared. HPLC/MS 1.54 min (A), [M+H]⁺ 643.0. ¹H NMR (DMSO-d₆, 400 MHz): δ 9.28 (br s, 1H), 8.30 (br d, J=9.3 Hz, 1H), 7.84 (br d, J=11.2 Hz, 1H), 7.39-7.43 (m, 1H), 7.34-7.39 (m, 1H), 7.29 (br s, 1H), 6.72-6.89 (m, 2H), 4.35 (br s, 2H), 3.87 (s, 3H), 3.57-3.72 (m, 2H), 3.16 (br s, 2H), 1.18 (s, 9H).

### Step D: 2-((2-(((2-(3-Amino-6-methoxypyridin-2-yl)ethyl)(tert-butoxycarbonyl)amino)-methyl)-4-chlorophenyl)amino)-5-fluoro-4-(trifluoromethyl)benzoic acid 66d

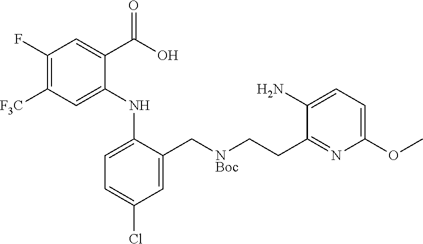 [SMILES: COc1ccc(N)c(CCN(Cc2cc(Cl)ccc2Nc2cc(C(F)(F)F)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-chlorophenyl)amino)-5-fluoro-4-(trifluoromethyl)benzoic acid 66d (3.46 g, 100% yield) as a foamy solid. HPLC/MS 1.13 min (A), [M+H]⁺ 613.1. ¹H NMR (DMSO-d₆, 400 MHz): δ 10.73-10.96 (m, 1H), 7.79 (br d, J=11.2 Hz, 1H), 7.28 (s, 2H), 7.08 (s, 1H), 6.96 (d, J=8.3 Hz, 1H), 6.85 (br d, J=1.5 Hz, 1H), 6.33 (d, J=8.8 Hz, 1H), 4.55 (br s, 2H), 4.36 (br s, 2H), 3.59 (s, 3H), 3.42-3.54 (m, 2H), 1.20-1.37 (m, 9H).

### Step E: tert-Butyl 8-chloro-2-fluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 66e

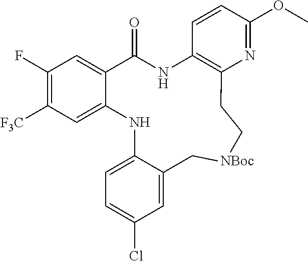 [SMILES: COc1ccc2c(n1)CCN(C(=O)OC(C)(C)C)Cc1cc(Cl)ccc1Nc1cc(C(F)(F)F)c(F)cc1C(=O)N2]

Following the procedure outlined in Example 41, Step E, substituting HATU with pyoxim, and stirring the reaction mixture at room temperature for 4 h, tert-butyl 8-chloro-2-fluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 66e (1.10 g, 34% yield) was prepared as a solid. HPLC/MS 1.43 min (A), [M+H]⁺ 595.1. ¹H NMR (DMSO-d₆, 400 MHz): δ 10.11-10.25 (m, 1H), 7.85 (br d, J=9.8 Hz, 1H), 7.77 (br d, J=8.8 Hz, 1H), 7.73 (s, 1H), 7.48-7.64 (m, 1H), 7.36 (d, J=2.9 Hz, 1H), 7.16 (dd, J=8.8, 2.4 Hz, 1H), 6.71 (d, J=8.8 Hz, 1H), 6.47-6.59 (m, 1H), 4.60 (s, 2H), 3.81 (s, 3H), 3.30 (br s, 2H), 2.61 (br s, 2H), 1.43 (br s, 9H).

### Step F: tert-Butyl 8-chloro-2-fluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 66f

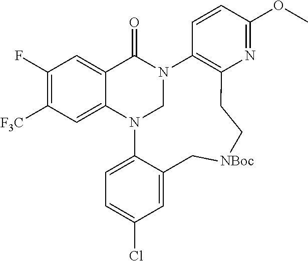 [SMILES: COc1ccc2c(n1)CCN(C(=O)OC(C)(C)C)Cc1cc(Cl)ccc1N1CN2C(=O)c2cc(F)c(C(F)(F)F)cc21]

Following the procedure outlined in Example 41, Step F, stirring the reaction at 85° C. for 36 h, tert-butyl 8-chloro-2-fluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 66f (825 mg, 74% yield) was prepared. HPLC/MS 1.45 min (A), [M+H]⁺ 607.1. ¹H NMR (DMSO-d₆, 400 MHz): δ 7.91 (d, J=10.3 Hz, 1H), 7.73 (d, J=8.8 Hz, 1H), 7.56-7.63 (m, 2H), 7.49 (d, J=8.3 Hz, 1H), 6.80 (d, J=8.3 Hz, 1H), 6.46 (d, J=5.4 Hz, 1H), 5.62 (d, J=11.7 Hz, 1H), 4.93 (d, J=11.7 Hz, 1H), 4.72 (d, J=14.2 Hz, 1H), 3.93 (d, J=14.2 Hz, 1H), 3.58-3.72 (m, 1H), 2.97 (td,J=11.4, 3.2 Hz, 1H), 2.66-2.78 (m, 1H), 2.56 (dt, J=11.7, 5.9 Hz, 1H), 1.45 (s, 9H).

### Step G: 8-Chloro-2-fluoro-3-(trifluoromethyl)-10,11,12,13-tetrahydro-19H-5,18-methanodibenzo[c,f]pyrido[3,2-i][1,5,9]triazacyclotridecine-15,19(14H)-dione hydrochloride

### Example 66

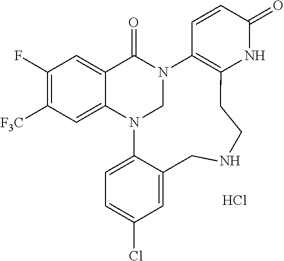 [SMILES: O=C1c2cc(F)c(C(F)(F)F)cc2N2CN1c1ccc(=O)[nH]c1CCNCc1cc(Cl)ccc12 ; Cl]

Following the procedure outlined in Example 41, Step G, stirring the reaction mixture at 85° C. for 12 h, 8-chloro-2-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (535 mg, 84% yield) was prepared as a white solid. HPLC/MS 0.72 min (A), [M+H]⁺ 493.1. ¹H NMR (DMSO-d₆, 400 MHz): δ 10.42 (br s, 1H), 8.90 (br s, 1H), 7.99 (d, J=2.93 Hz, 1H), 7.93 (d, J=10.76 Hz, 1H), 7.70-7.76 (m, 1H), 7.62 (d, J=8.31 Hz, 1H), 7.47 (d, J=9.29 Hz, 1H), 6.44 (d, J=5.38 Hz, 1H), 6.35 (d, J=9.78 Hz, 1H), 5.48 (d, J=11.74 Hz, 1H), 4.98 (d, J=11.74 Hz, 1H), 4.17-4.22 (m, 1H), 4.09 (br d, J=4.89 Hz, 1H), 3.19 (br d, J=6.36 Hz, 1H), 2.98-3.09 (m, 2H), 2.62-2.70 (m, 1H).

### Example 67

### Step A: 9-chloro-2,8-difluoro-15,19-dioxo-10,11,12,13,14,15-hexahydro-19H-5,18-methanodibenzo[c,f]pyrido[3,2-j][1,5,9]triazacyclotridecine-3-carbonitrile

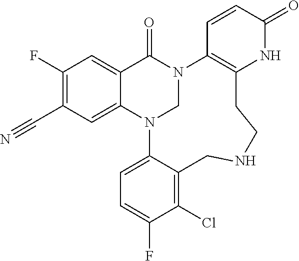 [SMILES: N#Cc1cc2c(cc1F)C(=O)N1CN2c2ccc(F)c(Cl)c2CNCCc2[nH]c(=O)ccc21]

Following the preparation outlined in Example 55, Step A, substituting Example 64 as the substrate and stirring the reaction at 70° C. for 24 h, 9-chloro-2,8-difluoro-15,19-dioxo-10,11,12,13,14,15-hexahydro-19H-5,18-methanodibenzo[c,f]pyrido[3,2-j][1,5,9]triazacyclo-tridecine-3-carbonitrile (33.7 mg, 31% yield) was prepared as a yellow solid. HPLC/MS 0.54 min (A), [M+H]⁺ 468.1. ¹H NMR (DMSO-d₆, 400 MHz): δ 11.6-12.3 (m, 1H), 7.85 (d, 1H, J=9.3 Hz), 7.6-7.6 (m, 1H), 7.5-7.6 (m, 1H), 7.41 (d, 1H, J=9.8 Hz), 6.98 (d, 1H, J=4.9 Hz), 6.23 (br d, 1H, J=9.3 Hz), 5.39 (d, 1H, J=10.3 Hz), 5.02 (d, 1H, J=9.8 Hz), 3.92 (br d, 1H, J=11.2 Hz), 3.69 (br d, 1H, J=10.8 Hz), 3.0-3.1 (m, 1H), 2.85 (br s, 2H), 2.23 (br s, 3H).

### Example 68

### 2,3,8-Trifluoro-9-methyl-10,11,12,13-tetrahydro-19H-5,18-methanodibenzo[c,f]pyrido[3,2-j][1,5,9]triazacyclotridecine-15,19(14H)-dione hydrochloride

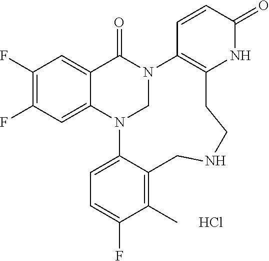 [SMILES: Cc1c(F)ccc2c1CNCCc1[nH]c(=O)ccc1N1CN2c2cc(F)c(F)cc2C1=O ; Cl]

### Step A: 2,3,8-Trifluoro-9-methyl-10,11,12,13-tetrahydro-19H-5,18-methanodibenzo-[c,f]pyrido[3,2-j][1,5,9]triazacyclotridecine-15,19(14H)-dione 68a

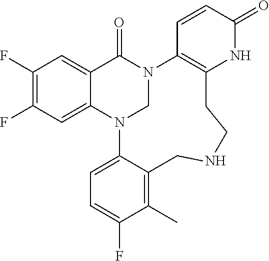 [SMILES: Cc1c(F)ccc2c1CNCCc1[nH]c(=O)ccc1N1CN2c2cc(F)c(F)cc2C1=O]

Following the preparation outlined in Example 61 Step A, substituting Example 64 as the substrate, 2,3,8-trifluoro-9-methyl-10,11,12,13-tetrahydro-19H-5,18-methanodibenzo[c,f]pyrido-[3,2-j][1,5,9]triazacyclotridecine-15,19(14H)-dione (33.7 mg, 31% yield) was prepared. HPLC/MS 0.53 min (A), [M+H]⁺ 441.2. ¹H NMR (DMSO-d₆, 400 MHz): δ 2.31 (d, J=1.96 Hz, 3H), 2.66-2.71 (m, 1H), 2.75-3.12 (m, 3H), 3.30 (s, 7H), 3.42-3.57 (m, 2H), 3.90 (s, 1H), 5.01 (d, J=9.78 Hz, 1H), 5.33 (d, J=9.78 Hz, 1H), 6.16-6.27 (m, 1H), 6.35 (dd, J=12.23, 6.36 Hz, 1H), 7.18-7.30 (m, 1H), 7.39 (br d, J=9.78 Hz, 2H), 7.72-7.88 (m, 1H), 11.80-11.90 (m, 1H).

### Step B: 2,3,8-Trifluoro-9-methyl-10,11,12,13-tetrahydro-19H-5,18-methanodibenzo-[c,f]pyrido[3,2-j][1,5,9]triazacyclotridecine-15,19(14H)-dione hydrochloride

### Example 68

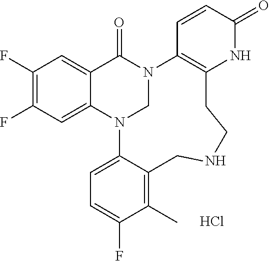 [SMILES: Cc1c(F)ccc2c1CNCCc1[nH]c(=O)ccc1N1CN2c2cc(F)c(F)cc2C1=O ; Cl]

Following the preparation outlined in Example 61 Step B, 2,3,8-trifluoro-9-methyl-10,11,12,13-tetrahydro-19H-5,18-methanodibenzo[c,f]pyrido[3,2-j][1,5,9]triazacyclotridecine-15,19(14H)-dione hydrochloride (26 mg, 68% yield) was prepared. HPLC/MS 0.61 min (A), [M+H]⁺ 441.1. ¹H NMR (CD₃OD, 400 MHz): δ 2.50 (d, J=2.45 Hz, 3H), 2.77-2.91 (m, 1H), 3.15-3.28 (m, 1H), 3.40-3.52 (m, 1H), 4.32-4.42 (m, 1H), 4.49 (br d, J=14.67 Hz, 1H), 4.91 (s, 1H), 5.57 (d, J=11.25 Hz, 1H), 6.13-6.20 (m, 1H), 6.60 (d, J=9.78 Hz, 1H), 7.39-7.48 (m, 2H), 7.64 (m, J=9.78 Hz, 1H), 7.91 (dd, J=10.27, 8.80 Hz, 1H).

### Example 69

### 2-Chloro-3,8,9-trifluoro-10,11,12,13-tetrahydro-19H-5,18-methanodibenzo[c,f]pyrido[3,2-j][1,5,9]triazacyclotridecine-15,19(14H)-dione hydrochloride

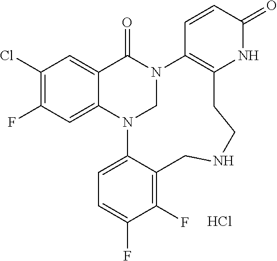 [SMILES: O=C1c2cc(Cl)c(F)cc2N2CN1c1ccc(=O)[nH]c1CCNCc1c2ccc(F)c1F ; Cl]

### Step A: Methyl 5-chloro-2-((3,4-difluoro-2-formylphenyl)amino)-4-fluorobenzoate 69a

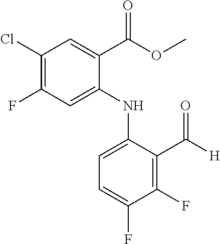 [SMILES: COC(=O)c1cc(Cl)c(F)cc1Nc1ccc(F)c(F)c1C=O]

Following the preparation outlined in Example 7, Step A, substituting ethyl 2-bromo-5-(trifluoromethyl)benzoate with methyl 2-bromo-5-chloro-4-fluorobenzoate, Int-3a with 6-bromo-2,3-difluorobenzaldehyde, BINAP with Xantphos, toluene with 1,4-dioxane, and stirring the reaction at 80° C. overnight, methyl 5-chloro-2-((3,4-difluoro-2-formylphenyl)amino)-4-fluorobenzoate 69a (906 mg, 35% yield) was prepared as a yellow solid. HPLC/MS 1.33 min (B), [M+H]⁺ 344.1. ¹H NMR (CDCl₃, 400 MHz): δ 11.43 (s, 1H), 10.43 (s, 1H), 8.11 (d, J=8.3 Hz, 1H), 7.22-7.38 (m, 3H), 3.99 (s, 3H).

### Step B: Methyl 2-((2-(((tert-butoxycarbonyl)(2-(6-methoxy-3-nitropyridin-2-yl)ethyl-amino)methyl)-3,4-difluorophenyl)amino)-5-chloro-4-fluorobenzoate 69b

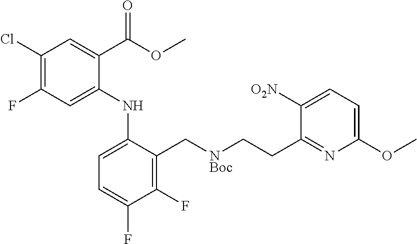 [SMILES: COC(=O)c1cc(Cl)c(F)cc1Nc1ccc(F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-chloro-2-((3,4-difluoro-2-formylphenyl)amino)-4-fluorobenzoate 69a, methyl 2-((2-(((tert-butoxycarbonyl)(2-(6-methoxy-3-nitropyridin-2-yl)ethyl)amino)methyl)-3,4-difluorophenyl)amino)-5-chloro-4-fluorobenzoate 69b (922 mg, 70% yield) was prepared as a clear oil. HPLC/MS 1.71 min (B), [M+H]⁺ 625.1. ¹H NMR (CDCl₃, 400 MHz): δ 9.37 (br s, 1H), 8.21 (d, J=9.3 Hz, 1H), 7.95 (br d, J=7.8 Hz, 1H), 7.15 (q, J=8.8 Hz, 1H), 7.05 (br dd, J=8.8, 3.4 Hz, 1H), 6.66 (br d, J=9.3 Hz, 1H), 6.38-6.45 (m, 1H), 4.60 (s, 2H), 3.96 (s, 3H), 3.90 (s, 3H), 3.64 (t, J=7.1 Hz, 2H), 3.26 (t, J=6.8 Hz, 2H), 1.31 (br s, 9H).

### Step C: 2-((2-(((tert-Butoxycarbonyl)(2-(6-methoxy-3-nitropyridin-2-yl)ethyl)amino)-methyl)-3,4-difluorophenyl)amino)-5-chloro-4-fluorobenzoic acid 69c

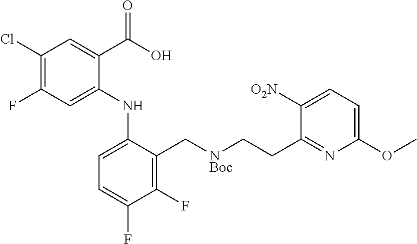 [SMILES: COc1ccc([N+](=O)[O-])c(CCN(Cc2c(Nc3cc(F)c(Cl)cc3C(=O)O)ccc(F)c2F)C(=O)OC(C)(C)C)n1]

Following the procedure outlined in Example 41, Step C, stirring the reaction mixture at RT for 2 h, 2-((2-(((tert-butoxycarbonyl)(2-(6-methoxy-3-nitropyridin-2-yl)ethyl)amino)methyl)-3,4-difluorophenyl)amino)-5-chloro-4-fluorobenzoic acid 69c (807 mg, 84%) as a yellow foam that solidified into a yellow powder. HPLC/MS 1.12 min (B), [M+H]⁺ 511.1. ¹H NMR (CDCl₃, 400 MHz): δ 9.48 (br s, 1H) 8.23 (br d, J=8.8 Hz, 1H), 7.99 (d, J=8.3 Hz, 1H), 7.18 (q, J=9.0 Hz, 1H), 7.08 (br dd, J=8.6, 3.7 Hz, 1H), 6.68 (br d, J=8.3 Hz, 1H), 6.42 (br d, J=11.7 Hz, 1H), 4.58 (s, 2H), 3.99 (s, 3H), 3.72 (br s, 2H), 3.30 (br t, J=6.8 Hz, 2H), 1.29-1.38 (m, 9H).

### Step D: 2-((2-(((2-(3-Amino-6-methoxypyridin-2-yl)ethyl)(tert-butoxycarbonyl)amino)-methyl)-3,4-difluorophenyl)amino)-5-chloro-4-fluorobenzoic acid 69d

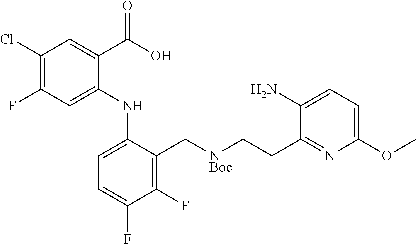 [SMILES: COc1ccc(N)c(CCN(Cc2c(Nc3cc(F)c(Cl)cc3C(=O)O)ccc(F)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3,4-difluorophenyl)amino)-5-chloro-4-fluorobenzoic acid 69d (471 mg, 74% yield) was prepared as a yellow solid. HPLC/MS 1.00 min (B), [M+H]⁺ 581.2. ¹H NMR (CD₃OD, 400 MHz): δ 7.84-8.00 (m, 1H), 6.97-7.25 (m, 3H), 6.40 (br d, J=8.3 Hz, 1H), 6.31 (br d, J=11.2 Hz, 1H), 4.53 (br s, 2H), 3.70 (s, 2H), 3.43 (br s, 2H), 2.79 (br s, 2H), 1.21-1.43 (m, 9H).

### Step E: tert-Butyl 2-chloro-3,8,9-trifluoro-15-methoxy-19-oxo-5,10,12,13,18,19-hexa-hydro-11H-dibenzo[c,f]pyrido[3,2-j][1,5,9]triazacyclotridecine-11-carboxylate 69e

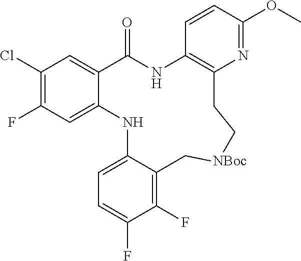 [SMILES: COc1ccc2c(n1)CCN(C(=O)OC(C)(C)C)Cc1c(ccc(F)c1F)Nc1cc(F)c(Cl)cc1C(=O)N2]

Following the procedure outlined in Example 41, Step E, stirring the reaction mixture at room temperature for 1 h, tert-butyl 2-chloro-3,8,9-trifluoro-15-methoxy-19-oxo-5,10,12,13,18,19-hexahydro-11H-dibenzo[c,f]pyrido[3,2-j][1,5,9]triazacyclotridecine-11-carboxylate 69e (278 mg, 60% yield) was prepared as a white solid. HPLC/MS 1.43 min (B), [M+H]⁺ 563.1. ¹H NMR (CDCl₃, 400 MHz): δ 10.44 (br s, 1H), 8.38 (d, J=8.3 Hz, 1H), 8.05 (d, J=8.8 Hz, 1H), 6.97 (d, J=8.8 Hz, 1H), 6.85 (q, J=9.3 Hz, 1H), 6.49 (d, J=8.8 Hz, 1H), 6.36 (s, 1H), 5.95 (br dd, J=8.8, 2.0 Hz, 1H), 4.49-4.90 (m, 2H), 3.48-3.83 (m, 5H), 2.22 (br s, 2H), 0.95 (s, 9H).

### Step F: tert-Butyl 2-chloro-3,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 69f

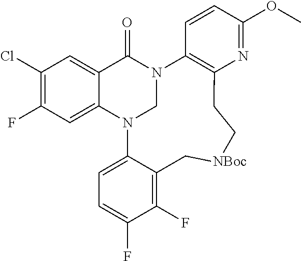 [SMILES: COc1ccc2c(n1)CCN(C(=O)OC(C)(C)C)Cc1c(ccc(F)c1F)N1CN2C(=O)c2cc(Cl)c(F)cc21]

Following the procedure outlined in Example 41, Step F, stirring the reaction at 90° C. overnight, tert-butyl 2-chloro-3,8,9-trifluoro-15-methoxy-19-oxo-12,13-dihydro-19H-5,18-methanodibenzo[c,f]pyrido[3,2-j][1,5,9]triazacyclotridecine-11(10H)-carboxylate 69f (71 mg, 18% yield) was prepared as a light tan solid. HPLC/MS 1.44 min (B), [M+H]⁺ 575.1. ¹H NMR (CD₃OD, 400 MHz): δ 7.96 (d, J=7.8 Hz, 1H), 7.52 (d, J=8.8 Hz, 1H), 7.28-7.39 (m, 1H), 7.13 (ddd, J=8.8, 4.4, 2.0 Hz, 1H), 6.64 (d, J=8.3 Hz, 1H), 6.17 (d, J=10.8 Hz, 1H), 5.53 (d, J=11.2 Hz, 1H), 5.04 (br d, J=14.2 Hz, 1H), 3.68-3.94 (m, 6H), 2.94 (br s, 1H), 2.53-2.69 (m, 2H), 1.38 (s, 9H).

### Step G: 2-Chloro-3,8,9-trifluoro-10,11,12,13-tetrahydro-19H-5,18-methanodibenzo-[c,f]pyrido[3,2-j][1,5,9]triazacyclotridecine-15,19(14H)-dione hydrochloride

### Example 69

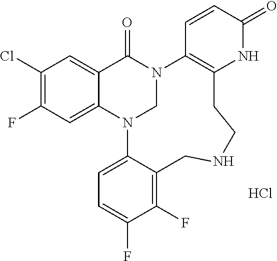 [SMILES: O=C1c2cc(Cl)c(F)cc2N2CN1c1ccc(=O)[nH]c1CCNCc1c2ccc(F)c1F ; Cl]

Following the procedure outlined in Example 41, Step G, stirring the reaction mixture at 90° C. for 6 h, 2-chloro-3,8,9-trifluoro-10,11,12,13-tetrahydro-19H-5,18-methanodibenzo-[c,f]pyrido[3,2-j][1,5,9]triazacyclotridecine-15,19(14H)-dione hydrochloride (1.64 g, 100% yield) was prepared as a white solid. HPLC/MS 0.99 min (B), [M+H]⁺ 461.1. ¹H NMR (DMSO-d₆, 400 MHz): δ 10.35-10.52 (m, 1H), 9.05-9.24 (m, 1H), 7.98 (d, J=8.3 Hz, 1H), 7.77 (br d, J=9.8 Hz, 1H), 7.39-7.49 (m, 2H), 6.28-6.37 (m, 2H), 5.45 (d, J=11.7 Hz, 1H), 5.05 (d, J=11.7 Hz, 1H), 4.19 (br s, 1H), 4.11 (br s, 1H), 3.16-3.33 (m, 1H), 3.02 (br t, J=8.8 Hz, 2H), 2.69 (br s, 1H).

### Example 70

### Step A: 2,3,8-Trifluoro-9-methoxy-10,11,12,13-tetrahydro-19H-5,18-methanodibenzo-[c,f]pyrido[3,2-j][1,5,9]triazacyclotridecine-15,19(14H)-dione, Formic acid salt

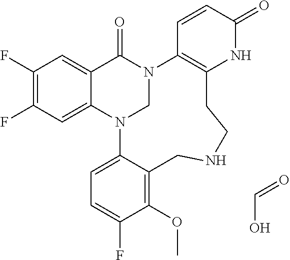 [SMILES: COc1c(F)ccc2c1CNCCc1[nH]c(=O)ccc1N1CN2c2cc(F)c(F)cc2C1=O ; O=CO]

A mixture of 9-chloro-2,3,8-trifluoro-10,11,12,13-tetrahydro-19H-5,18-methanodibenzo-[c,]pyrido[3,2-j][1,5,9]triazacyclotridecine-15,19(14H)-dione hydrochloride Example 64 (250.0 mg, 503 μmol), sodium 2-methylpropan-2-olate (169.1 mg, 1.76 mmol), and methanesulfonato[2-(di-1-adamantylphosphino)-3,6-dimethoxy-2′,4′,6′-tri-i-propyl-1,1′-biphenyl](2′-amino-1,1′-biphenyl-2-yl)palladium(II) (101.7 mg, 101 mol) was purged with nitrogen. Dioxane (4.0 mL) and MeOH (1.0 mL) were added and the reaction mixture was heated at 80° C. for 17 h, diluted with EtOAc, washed with H₂O, dried over Na₂SO₄, filtered and concentrated under reduced pressure. The crude residue was purified by reverse-phase semi-prep HPLC (XSELECT CSH C18 column), eluting with 20-99% AcCN in 10 mM ammonium bicarbonate in H₂O adjusted to pH 10 with ammonia to afford a partially pure product. The material was further purified by reverse-phase semi-prep HPLC (XSELECT CSH C18 column), eluting with 15-99% AcCN (0.1% formic acid) in water (0.1% formic acid) to afford 2,3,8-trifluoro-9-methoxy-10,11,12,13-tetrahydro-19H-5,18-methanodibenzo[c,f]pyrido[3,2-j][1,5,9]triazacyclotridecine-15,19(14H)-dione, formic acid salt (33.7 mg, 31% yield) as a white powder. HPLC/MS 0.53 min (G), [M+H]⁺ 457.17. ¹H NMR (DMSO-d₆, 400 MHz): δ 2.16-2.31 (m, 1H), 2.85 (br s, 4H), 3.67 (br d, J=2.93 Hz, 3H), 3.93 (d, J=1.47 Hz, 4H), 5.04 (d, J=9.78 Hz, 1H), 5.31 (d, J=9.78 Hz, 1H), 6.21 (br d, J=9.29 Hz, 1H), 6.41 (br d, J=5.87 Hz, 1H), 7.22-7.33 (m, 1H), 7.31-7.43 (m, 2H), 7.74-7.86 (m, 1H), 8.15 (s, 1H), 11.77-11.99 (m, 1H).

### Example 71

### 2,8-Difluoro-9-methyl-15,19-dioxo-10,11,12,13,14,15-hexahydro-19H-518-methanodibenzo-[c,f]pyrido[3,2-j][1,5,9]triazacyclotridecine-3-carbonitrile hydrochloride

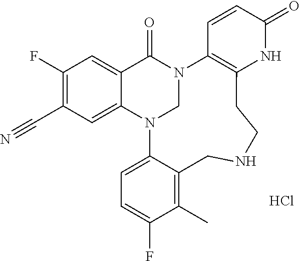 [SMILES: Cc1c(F)ccc2c1CNCCc1[nH]c(=O)ccc1N1CN2c2cc(C#N)c(F)cc2C1=O ; Cl]

### Step A: 2,8-Difluoro-9-methyl-15,19-dioxo-10,11,12,13,14,15-hexahydro-19H-5,18-methanodibenzo-[c,f]pyrido[3,2-j][1,5,9]triazacyclotridecine-3-carbonitrile 71a

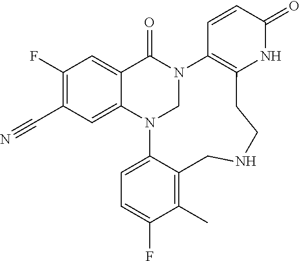 [SMILES: Cc1c(F)ccc2c1CNCCc1[nH]c(=O)ccc1N1CN2c2cc(C#N)c(F)cc2C1=O]

Following the procedure outlined in Example 55a, using 2,3,8-trifluoro-9-methyl-10,11,12,13-tetrahydro-19H-5,18-methanodibenzo[c,f]pyrido[3,2-j][1,5,9]triazacyclotridecine-15,19(14H)-dione hydrochloride Example 68 as the substrate and heating at 70° C. for 21 h, 2,8-difluoro-9-methyl-15,19-dioxo-10,11,12,13,14,15-hexahydro-19H-5,18-methanodibenzo-[c,f]pyrido[3,2-j][1,5,9]triazacyclotridecine-3-carbonitrile 71a (26 mg, 53% yield) was prepared as a pale yellow solid. HPLC/MS 0.91 min (B), [M+H]⁺ 448.1. ¹H NMR (DMSO-d₆, 400 MHz): δ 2.17-2.29 (m, 1H), 2.32 (d, J=1.47 Hz, 4H), 2.77-3.14 (m, 4H), 3.48 (br d, J=10.76 Hz, 1H), 3.80-3.97 (m, 1H), 5.03 (br d, J=9.78 Hz, 1H), 5.37 (br d, J=9.78 Hz, 1H), 6.22 (br d, J=8.80 Hz, 1H), 6.86 (d, J=4.89 Hz, 1H), 7.25 (s, 1H), 7.40 (br d, J=9.29 Hz, 2H), 7.84 (d, J=9.29 Hz, 1H), 11.87 (br s, 1H).

### Step B: 2,8-Difluoro-9-methyl-15,19-dioxo-10,11,12,13,14,15-hexahydro-19H-5,18-methanodibenzo-[c,f]pyrido[3,2-j][1,5,9]triazacyclotridecine-3-carbonitrile hydrochloride

### Example 71

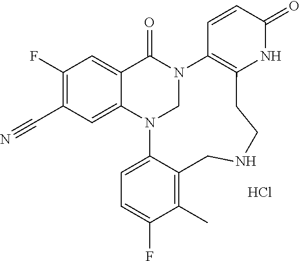 [SMILES: Cc1c(F)ccc2c1CNCCc1[nH]c(=O)ccc1N1CN2c2cc(C#N)c(F)cc2C1=O ; Cl]

Following the procedure outlined in Example 41, Step G, stirring the reaction mixture at room temperature for 5 min, 2,8-difluoro-9-methyl-15,19-dioxo-10,11,12,13,14,15-hexahydro-19H-5,18-methanodibenzo[c,f]pyrido[3,2-j][1,5,9]triazacyclotridecine-3-carbonitrile hydrochloride (16 mg, 57% yield) was prepared. HPLC/MS 0.91 min (B), [M+H]⁺ 448.1. ¹H NMR (DMSO-d₆, 400 MHz): δ 2.44 (s, 3H), 3.00-3.27 (m, 3H), 4.04-4.36 (m, 2H), 4.95 (br d, J=11.25 Hz, 1H), 5.43 (br d, J=11.25 Hz, 1H), 6.26-6.40 (m, 1H), 6.70 (d, J=4.89 Hz, 1H), 7.36-7.60 (m, 3H), 7.92 (d, J=8.80 Hz, 1H), 8.23-8.43 (m, 1H), 9.47-9.71 (m, 1H), 11.94 (br s, 1H).

### Example 72

### Step A: 2-Chloro-8,9-difluoro-15,19-dioxo-10,11,12,13,14,15-hexahydro-19H-5,18-methanodibenzo-[c,f]pyrido[3,2-j][1,5,9]triazacyclotridecine-3-carbonitrile hydrochloride

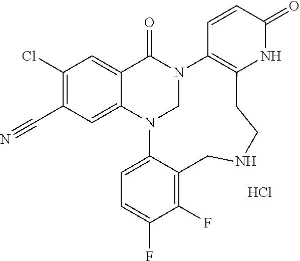 [SMILES: N#Cc1cc2c(cc1Cl)C(=O)N1CN2c2ccc(F)c(F)c2CNCCc2[nH]c(=O)ccc21 ; Cl]

Following the procedure outlined in Example 55a, using 2-chloro-3,8,9-trifluoro-10,11,12,13-tetrahydro-19H-5,18-methanodibenzo[c,f]pyrido[3,2-j][1,5,9]triazacyclotridecine-15,19(14H)-dione hydrochloride Example 69 as the substrate and heating at 80° C. over the weekend, 2-chloro-8,9-difluoro-15,19-dioxo-10,11,12,13,14,15-hexahydro-19H-5,18-methano-dibenzo[c,f]pyrido[3,2-j][1,5,9]triazacyclotridecine-3-carbonitrile hydrochloride (26 mg, 53% yield) was prepared as a pale yellow solid. HPLC/MS 0.90 min (B), [M+H]⁺ 468.1. ¹H NMR (CDCl₃, 400 MHz): δ 8.18 (s, 1H), 7.27-7.34 (m, 2H), 7.08-7.18 (m, 1H), 6.82 (s, 1H), 6.44 (d, J=9.3 Hz, 1H), 5.31 (d, J=9.3 Hz, 1H), 5.23 (d, J=9.8 Hz, 1H), 3.74-3.96 (m, 2H), 3.37 (br d, J=7.3 Hz, 2H), 3.10 (br s, 2H).

### Example 73

### 2,8-Difluoro-9-methoxy-15,19-dioxo-10,11,12,13,14,15-hexahydro-19H-5,18-methanodibenzo-[c,f]pyrido[3,2-j][1,5,9]triazacyclotridecine-3-carbonitrile hydrochloride

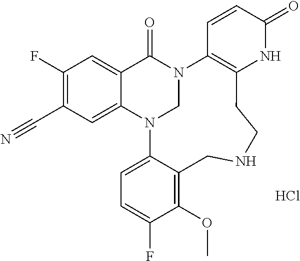 [SMILES: COc1c(F)ccc2c1CNCCc1[nH]c(=O)ccc1N1CN2c2cc(C#N)c(F)cc2C1=O ; Cl]

### Step A: 2,8-Difluoro-9-methoxy-15,19-dioxo-10,11,12,13,14,15-hexahydro-19H-5,18-methanodibenzo-[c,f]pyrido[3,2-j][1,5,9]triazacyclotridecine-3-carbonitrile 73a

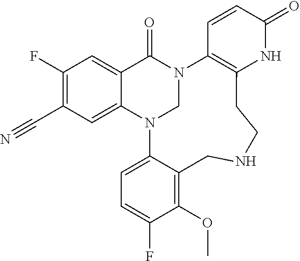 [SMILES: COc1c(F)ccc2c1CNCCc1[nH]c(=O)ccc1N1CN2c2cc(C#N)c(F)cc2C1=O]

Following the procedure outlined in Example 55a, using 2,3,8-trifluoro-9-methoxy-10,11,12,13-tetrahydro-19H-5,18-methanodibenzo[c,f]pyrido[3,2-j][1,5,9]triazacyclotridecine-15,19(14H)-dione, formate salt Example 70 as the substrate and heating at 70° C. for 17 h, 2,8-difluoro-9-methoxy-15,19-dioxo-10,11,12,13,14,15-hexahydro-19H-5,18-methanodibenzo[c,f]-pyrido[3,2-j][1,5,9]triazacyclotridecine-3-carbonitrile 73a (22 mg, 43% yield) was prepared as a white solid. HPLC/MS 0.87 min (B), [M+H]⁺ 464.1. ¹H NMR (CD₃OD, 400 MHz): δ 2.29-2.50 (m, 1H), 2.95-3.21 (m, 4H), 3.72-3.90 (m, 2H), 4.03 (d, J=1.96 Hz, 3H), 5.20 (d, J=9.78 Hz, 1H), 5.39 (d, J=9.78 Hz, 1H), 6.44 (d, J=9.29 Hz, 1H), 6.85 (d, J=4.89 Hz, 1H), 7.18-7.35 (m, 2H), 7.52 (d, J=9.29 Hz, 1H), 7.88 (d, J=8.80 Hz, 1H).

### Step B: 2,8-Difluoro-9-methoxy-15,19-dioxo-10,11,12,13,14,15-hexahydro-19H-5,18-methanodibenzo-[c,f]pyrido[3,2-j][1,5,9]triazacyclotridecine-3-carbonitrile hydrochloride

### Example 73

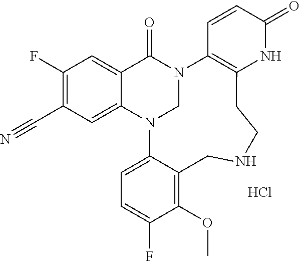 [SMILES: COc1c(F)ccc2c1CNCCc1[nH]c(=O)ccc1N1CN2c2cc(C#N)c(F)cc2C1=O ; Cl]

Following the procedure outlined in Example 41, Step G, stirring the reaction mixture at room temperature for 5 min, 2,8-difluoro-9-methoxy-15,19-dioxo-10,11,12,13,14,15-hexahydro-19H-5,18-methanodibenzo[c,f]pyrido[3,2-j][1,5,9]triazacyclotridecine-3-carbonitrile hydrochloride (23 mg, 96% yield) was prepared. HPLC/MS 0.56 min (A), [M+H]⁺ 464.1. ¹H NMR (DMSO-d₆, 400 MHz): δ 2.92-3.17 (m, 3H), 4.10 (br d, J=2.45 Hz, 6H), 4.79-4.95 (m, 1H), 5.42 (br d, J=12.23 Hz, 1H), 6.26-6.41 (m, 1H), 6.86 (d, J=5.38 Hz, 1H), 7.29 (dd, J=9.05, 4.16 Hz, 1H), 7.39-7.51 (m, 1H), 7.61 (dd, J=11.49, 9.05 Hz, 1H), 7.91 (d, J=9.29 Hz, 1H), 8.29-8.46 (m, 1H), 9.31-9.50 (m, 1H), 11.86-12.00 (m, 1H).

### Example 74

### 8-Chloro-2,3,9-trifluoro-10,11,12,13-tetrahydro-19H-5,18-methanodibenzo-[c,f]pyrido[3,2-j][1,5,9]triazacyclotridecine-15,19(14H)-dione hydrochloride

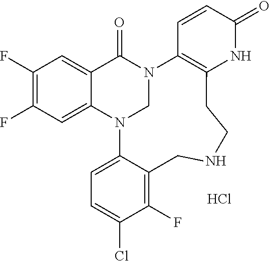 [SMILES: O=C1c2cc(F)c(F)cc2N2CN1c1ccc(=O)[nH]c1CCNCc1c2ccc(Cl)c1F ; Cl]

### Step A: Methyl 2-((4-chloro-3-fluoro-2-formylphenyl)amino)-4,5-difluorobenzoate 74a

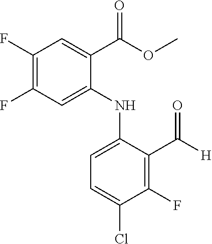 [SMILES: COC(=O)c1cc(F)c(F)cc1Nc1ccc(Cl)c(F)c1C=O]

Following the preparation outlined in Example 7, Step A, substituting ethyl 2-bromo-5-(trifluoromethyl)benzoate with methyl 2-amino-4,5-difluorobenzoate, Int-3a with 6-bromo-3-chloro-2-fluorobenzaldehyde, BINAP with Xantphos, toluene with 1,4-dioxane, and stirring the reaction at 80° C. for 2 h, methyl 2-((4-chloro-3-fluoro-2-formylphenyl)amino)-4,5-difluorobenzoate 74a (4.33 g, 99% yield) was prepared as a yellow solid. HPLC/MS 1.31 min (A), [M+H]⁺ 344.0. ¹H NMR (DMSO-d₆, 400 MHz): δ 3.87 (s, 3H), 7.29 (d, J=9.29 Hz, 1H), 7.60-7.78 (m, 2H), 7.91-8.00 (m, 1H), 10.30 (s, 1H), 11.22 (s, 1H).

### Step B: Methyl 2-((2-(((tert-butoxycarbonyl)(2-(6-methoxy-3-nitropyridin-2-yl)ethyl-amino)methyl)-4-chloro-3-fluorophenyl)amino)-4,5-difluorobenzoate 74b

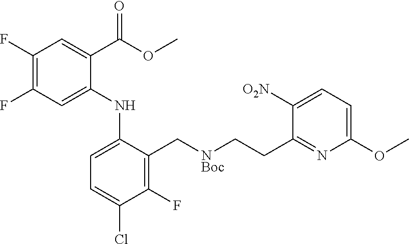 [SMILES: COC(=O)c1cc(F)c(F)cc1Nc1ccc(Cl)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((4-chloro-3-fluoro-2-formylphenyl)amino)-4,5-difluorobenzoate 74a, methyl 2-((2-(((tert-butoxycarbonyl)(2-(6-methoxy-3-nitropyridin-2-yl)ethyl)amino)methyl)-4-chloro-3-fluorophenyl)amino)-4,5-difluorobenzoate 74b (5.18 g, 60% yield) was prepared as a pale yellow solid. HPLC/MS 1.66 min (B), [M+H]⁺ 625.1. ¹H NMR (CDCl₃, 400 MHz): δ 1.36 (s, 9H), 3.32 (t, J=6.85 Hz, 3H), 3.69 (t, J=7.09 Hz, 2H), 3.92 (s, 3H), 4.00-4.06 (m, 4H), 4.67 (s, 2H), 6.70 (br d, J=8.80 Hz, 2H), 7.07-7.11 (m, 1H), 7.32-7.41 (m, 1H), 7.66-7.89 (m, 1H), 8.25 (d, J=9.29 Hz, 1H), 9.30 (br s, 1H).

### Step C: 2-((2-(((tert-Butoxycarbonyl)(2-(6-methoxy-3-nitropyridin-2-yl)ethyl)amino)-methyl)-4-chloro-3-fluorophenyl)amino)-4,5-difluorobenzoic acid 74c

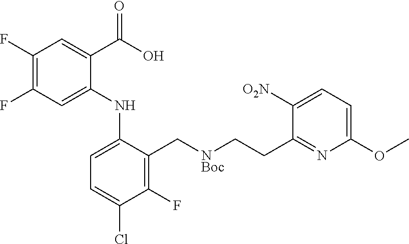 [SMILES: COc1ccc([N+](=O)[O-])c(CCN(Cc2c(Nc3cc(F)c(F)cc3C(=O)O)ccc(Cl)c2F)C(=O)OC(C)(C)C)n1]

Following the procedure outlined in Example 41, Step C, stirring the reaction mixture at room temperature for 19 h, 2-((2-(((tert-butoxycarbonyl)(2-(6-methoxy-3-nitropyridin-2-yl)ethyl)amino)methyl)-4-chloro-3-fluorophenyl)amino)-4,5-difluorobenzoic acid 74c (5.09 g, 94%) as a light yellow foam. HPLC/MS 1.10 min (A), [M+H]⁺ 611.1. ¹H NMR (DMSO-d₆, 400 MHz): δ 1.13 (s, 9H), 3.06-3.23 (m, 2H), 3.33 (br s, 5H), 3.59 (br t, J=6.36 Hz, 2H), 3.93 (br s, 3H), 4.51 (br s, 2H), 6.54-6.78 (m, 1H), 6.87 (br d, J=6.36 Hz, 1H), 7.21 (d, J=8.31 Hz, 1H), 7.54 (t, J=8.56 Hz, 1H), 7.79 (dd, J=11.25, 9.29 Hz, 1H), 8.33 (br d, J=9.29 Hz, 1H), 9.47 (s, 1H), 13.16-13.80 (m, 2H).

### Step D: 2-((2-(((2-(3-Amino-6-methoxypyridin-2-yl)ethyl)(tert-butoxycarbonyl)amino)-methyl)-4-chloro-3-fluorophenyl)amino)-4,5-difluorobenzoic acid 74d

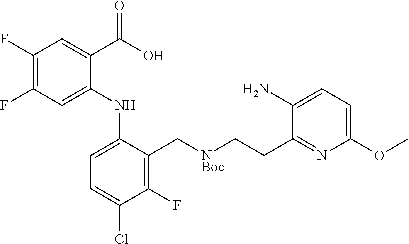 [SMILES: COc1ccc(N)c(CCN(Cc2c(Nc3cc(F)c(F)cc3C(=O)O)ccc(Cl)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-chloro-3-fluorophenyl)-amino)-4,5-difluorobenzoic acid 74d (5.07 g, 99% yield) was prepared as a light brown solid. HPLC/MS 1.10 min (A), [M+H]⁺ 581.1. ¹H NMR (DMSO-d₆, 400 MHz): δ 1.20 (s, 8H), 2.77 (br t, J=6.36 Hz, 2H), 3.34-3.60 (m, 6H), 3.74 (br s, 4H), 4.47 (s, 2H), 6.48-6.74 (m, 2H), 7.18-7.23 (m, 1H), 7.24-7.39 (m, 1H), 7.54 (s, 1H), 7.81 (dd, J=11.25, 9.29 Hz, 1H), 9.47 (s, 1H).

### Step E: tert-Butyl 8-chloro-2,3,9-trifluoro-15-methoxy-19-oxo-5,10,12,13,18,19-hexa-hydro-11H-dibenzo[c,f]pyrido[3,2-j][1,5,9]triazacyclotridecine-11-carboxylate 74e

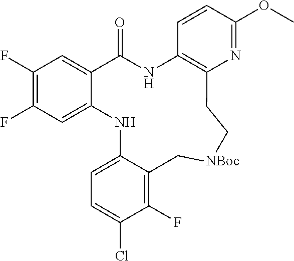 [SMILES: COc1ccc2c(n1)CCN(C(=O)OC(C)(C)C)Cc1c(ccc(Cl)c1F)Nc1cc(F)c(F)cc1C(=O)N2]

Following the procedure outlined in Example 41, Step E, stirring the reaction mixture at room temperature for 1 h, tert-butyl 8-chloro-2,3,9-trifluoro-15-methoxy-19-oxo-5,10,12,13,18,19-hexahydro-11H-dibenzo[c,f]pyrido[3,2-j][1,5,9]triazacyclotridecine-11-carboxylate 74e and used as is in the next step HPLC/MS 1.42 min (A), [M+H]⁺ 563.1.

### Step F: tert-Butyl 8-chloro-2,3,9-trifluoro-15-methoxy-19-oxo-12,13-dihydro-19H-5,18-methanodibenzo[c,f]pyrido[3,2-j][1,5,9]triazacyclotridecine-11(10H)-carboxylate 74f

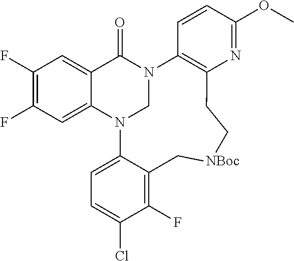 [SMILES: COc1ccc2c(n1)CCN(C(=O)OC(C)(C)C)Cc1c(ccc(Cl)c1F)N1CN2C(=O)c2cc(F)c(F)cc21]

Following the procedure outlined in Example 41, Step F, stirring the reaction at 80° C. for 96 h, tert-butyl 8-chloro-2,3,9-trifluoro-15-methoxy-19-oxo-12,13-dihydro-19H-5,18-methano-dibenzo[c,f]pyrido[3,2-j][1,5,9]triazacyclotridecine-11(10H)-carboxylate 74f (2.26 g, 68% yield) was prepared as a grey-white solid. HPLC/MS 138 min (A), [M+H]⁺ 575.1. ¹H NMR (CDCl₃, 400 MHz): δ 1.54 (s, 10H), 2.44-2.66 (m, 1H), 2.79-2.96 (m, 1H), 2.99-3.12 (m, 1H), 3.91-3.98 (m, 3H), 4.03 (br d, J=14.18 Hz, 2H), 4.57 (d, J=10.76 Hz, 1H), 5.18 (br d, J=14.18 Hz, 1H), 5.61 (d, J=11.25 Hz, 1H), 6.15 (dd, J=11.25, 6.36 Hz, 1H), 6.69 (d, J=8.80 Hz, 1H), 7.00 (dd, J=8.56, 1.22 Hz, 1H), 7.41-7.61 (m, 2H), 7.96 (dd, J=10.03, 8.56 Hz, 1H).

### Step G: 8-Chloro-2,3,9-trifluoro-10,11,12,13-tetrahydro-19H-5,18-methanodibenzo-[c,f]pyrido[3,2-j][1,5,9]triazacyclotridecine-15,19(14H)-dione hydrochloride

### Example 74

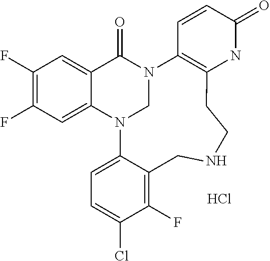 [SMILES: O=C1c2cc(F)c(F)cc2N2CN1c1ccc(=O)[nH]c1CCNCc1c2ccc(Cl)c1F ; Cl]

Following the procedure outlined in Example 41, Step G, stirring the reaction mixture at 90° C. for 17 h, 8-chloro-2,3,9-trifluoro-10,11,12,13-tetrahydro-19H-5,18-methanodibenzo-[c,f]pyrido[3,2-j][1,5,9]triazacyclotridecine-15,19(14H)-dione hydrochloride (848 mg, 97% yield) was prepared as a white solid. HPLC/MS 0.59 min (A), [M+H]⁺ 461.1. ¹H NMR (DMSO-d₆, 400 MHz): δ 2.69 (br s, 1H), 3.01 (br d, J=7.00 Hz, 2H), 3.24 (br d, J=12.51 Hz, 1H), 3.99-4.68 (m, 12H), 4.99 (d, J=11.51 Hz, 1H), 5.46 (d, J=11.51 Hz, 1H), 6.27-6.52 (m, 2H), 7.46 (t, J=8.25 Hz, 2H), 7.81-8.01 (m, 2H), 8.95 (br d, J=5.00 Hz, 1H), 10.05 (br s, 1H), 11.52-12.25 (m, 1H).

### Example 75

### 9-(Difluoromethoxy)-2,3-difluoro-10,11,12,13-tetrahydro-19H-5,18-methanodibenzo[c,f]pyrido[3,2-j][1,5,9]triazacyclotridecine-15,19(14H)-dione hydrochloride

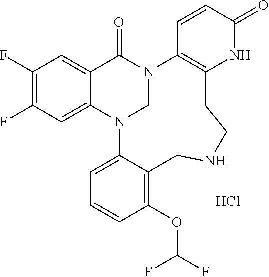 [SMILES: O=C1c2cc(F)c(F)cc2N2CN1c1ccc(=O)[nH]c1CCNCc1c(OC(F)F)cccc12 ; Cl]

### Step A: Methyl 2-((3-(difluoromethoxy)-2-formylphenyl)amino)-4,5-difluorobenzoate 75a

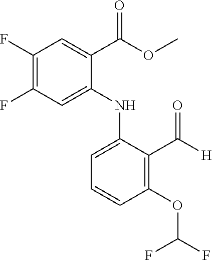 [SMILES: COC(=O)c1cc(F)c(F)cc1Nc1cccc(OC(F)F)c1C=O]

Following the preparation outlined in Example 7, Step A, substituting ethyl 2-bromo-5-(trifluoromethyl)benzoate with methyl 2-amino-4,5-difluorobenzoate, Int-3a with 2-bromo-6-(difluoromethoxy)benzaldehyde, BINAP with Xantphos, toluene with 1,4-dioxane, and stirring the reaction at 90° C. for 22 h, methyl 2-((3-(difluoromethoxy)-2-formylphenyl)amino)-4,5-difluoro-benzoate 75a (4.05 g, 74% yield) was prepared as a yellow solid. HPLC/MS 1.26 min (B), [M+H]⁺ 358.1. ¹H NMR (DMSO-d₆, 400 MHz): δ 11.35 (s, 1H), 10.34 (s, 1H), 7.9-8.0 (m, 1H), 7.5-7.7 (m, 2H), 7.2-7.4 (m, 2H), 6.80 (d, 1H, J=8.2 Hz), 3.86 (s, 3H).

### Step B: Methyl 2-((2-(((tert-butoxycarbonyl)(2-(6-methoxy-3-nitropyridin-2-yl)ethyl-amino)methyl)-3-(difluoromethoxy)phenyl)amino)-4,5-difluorobenzoate 75b

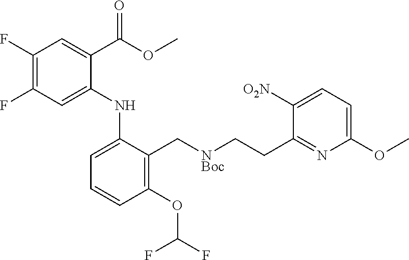 [SMILES: COC(=O)c1cc(F)c(F)cc1Nc1cccc(OC(F)F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3-(difluoro-methoxy)-2-formylphenyl)amino)-4,5-difluorobenzoate 75a, using a solvent combination of 10:5:3 DCE-MeCN-DMF, replacing NaBH(OAc)₃ with NaCNBH₃ and stirring at 60° C. for 19 h, methyl 2-((2-(((tert-butoxycarbonyl)(2-(6-methoxy-3-nitropyridin-2-yl)ethyl)amino)methyl)-3-(difluoromethoxy)-phenyl)amino)-4,5-difluorobenzoate 75b (4.84 g, 63% yield) was prepared as a light yellow solid. HPLC/MS 1.59 min (B), [M+H]⁺ 639.1. ¹H NMR (DMSO-d₆, 400 MHz): δ 9.17 (s, 1H), 8.3-8.4 (m, 1H), 7.7-7.8 (m, 1H), 7.0-7.5 (m, 4H), 6.8-6.9 (m, 1H), 6.62 (br s, 1H), 4.52 (s, 2H), 3.8-3.9 (m, 3H), 3.53 (br s, 2H), 3.3-3.4 (m, 3H), 3.1-3.2 (m, 2H), 1.0-1.2 (m, 9H).

### Step C: 2-((2-(((tert-Butoxycarbonyl)(2-(6-methoxy-3-nitropyridin-2-yl)ethyl)amino)-methyl)-3-(difluoromethoxy)phenyl)amino)-4,5-difluorobenzoic acid 75c

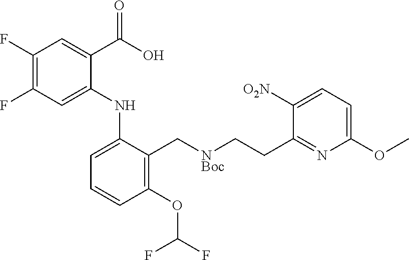 [SMILES: COc1ccc([N+](=O)[O-])c(CCN(Cc2c(Nc3cc(F)c(F)cc3C(=O)O)cccc2OC(F)F)C(=O)OC(C)(C)C)n1]

Following the procedure outlined in Example 41, Step C, stirring the reaction mixture at 50° C. for 17 h, 2-((2-(((tert-butoxycarbonyl)(2-(6-methoxy-3-nitropyridin-2-yl)ethyl)amino)-methyl)-3-(difluoromethoxy)phenyl)amino)-4,5-difluorobenzoic acid 75c (4.87 g, 100% yield) was prepared as a yellow solid. HPLC/MS 1.05 min (B), [M+H]⁺ 625.2. ¹H NMR (DMSO-d₆, 400 MHz): δ 9.49 (br s, 1H), 8.3-8.4 (m, 1H), 7.7-7.8 (m, 1H), 7.0-7.5 (m, 4H), 6.8-6.9 (m, 1H), 6.5-6.7 (m, 1H), 4.53 (s, 2H), 3.9-4.0 (m, 3H), 3.2-3.4 (m, 2H), 3.13 (br t, 2H, J=6.4 Hz), 1.0-1.2 (m, 9H).

### Step D: 2-((2-(((2-(3-Amino-6-methoxypyridin-2-yl)ethyl)(tert-butoxycarbonyl)amino)-methyl)-3-(difluoromethoxy)phenyl)amino)-4,5-difluorobenzoic acid 75d

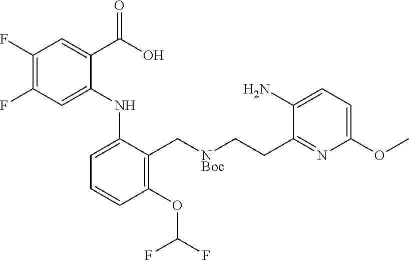 [SMILES: COc1ccc(N)c(CCN(Cc2c(Nc3cc(F)c(F)cc3C(=O)O)cccc2OC(F)F)C(=O)OC(C)(C)C)n1]

Following the procedure outlined in Example 44, Step D, stirring the reaction mixture under H₂ atmosphere for 17 h, 2-((2-(((2-(3-amino-6-methoxypyridin-2-yl)ethyl)(tert-butoxy-carbonyl)amino)methyl)-3-(difluoromethoxy)phenyl)-amino)-4,5-difluorobenzoic acid 75d (4.35 g, 100% yield) as a tan solid. HPLC/MS 0.96 min (B), [M+H]⁺ 595.1. ¹H NMR (DMSO-d₆, 400 MHz): δ 9.55 (br s, 1H), 7.7-7.8 (m, 1H), 7.4-7.5 (m, 1H), 7.0-7.3 (m, 3H), 6.9-7.0 (m, 1H), 6.4-6.6 (m, 1H), 6.3-6.4 (m, 1H), 4.45 (s, 2H), 3.64 (s, 3H), 3.37 (br s, 2H), 2.64 (br s, 2H), 1.2-1.3 (m, 9H) (three protons hidden).

### Step E: tert-Butyl 9-(difluoromethoxy)-2,3-difluoro-15-methoxy-19-oxo-5,10,12,13,18,19-hexahydro-11H-dibenzo[c,f]pyrido[3,2-j][1,5,9]triazacyclotridecine-11-carboxylate 75e

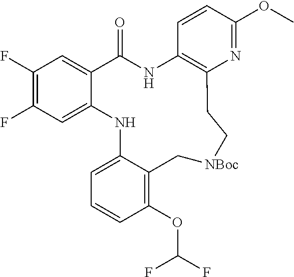 [SMILES: COc1ccc2c(n1)CCN(C(=O)OC(C)(C)C)Cc1c(cccc1OC(F)F)Nc1cc(F)c(F)cc1C(=O)N2]

Following the procedure outlined in Example 41, Step E, substituting HATU with pyoxim, and stirring the reaction mixture at room temperature for 70 min, tert-butyl 9-(difluoromethoxy)-2,3-difluoro-15-methoxy-19-oxo-5,10,12,13,18,19-hexahydro-11H-dibenzo[c,f]pyrido[3,2-j]-[1,5,9]triazacyclotridecine-11-carboxylate 75e (1.81 g, 44% yield) was prepared as a light pink solid. HPLC/MS 1.40 min (B), [M+H]⁺ 577.1. ¹H NMR (DMSO-d₆, 400 MHz): δ 10.27 (br s, 1H), 7.8-8.0 (m, 2H), 7.77 (br s, 1H), 7.41 (br s, 1H), 6.9-7.3 (m, 2H), 6.6-6.7 (m, 2H), 6.21 (d, 1H, J=7.8 Hz), 4.74 (br s, 2H), 3.79 (s, 3H), 2.9-3.2 (m, 2H), 2.73 (br s, 2H), 1.38 (br s, 9H).

### Step F: tert-Butyl 9-(difluoromethoxy)-2,3-difluoro-15-methoxy-19-oxo-12,13-dihydro-19H-5,18-methanodibenzo[c,f]pyrido[3,2-j][1,5,9]triazacyclotridecine-11(10H)-carboxylate 75f

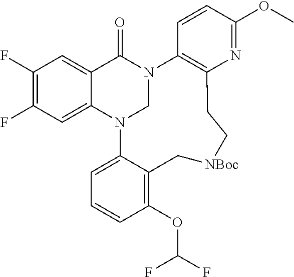 [SMILES: COc1ccc2c(n1)CCN(C(=O)OC(C)(C)C)Cc1c(OC(F)F)cccc1N1CN2C(=O)c2cc(F)c(F)cc21]

Following the procedure outlined in Example 41, Step F, stirring the reaction at 90° C. for 72 h, tert-butyl 9-(difluoromethoxy)-2,3-difluoro-15-methoxy-19-oxo-12,13-dihydro-19H-5,18-methanodibenzo-[c,f]pyrido[3,2-j][1,5,9]triazacyclotridecine-11(10H)-carboxylate 75f (1.56 g, 84% yield) was prepared as a yellow solid. HPLC/MS 1.34 min (B), [M+H]⁺ 589.1. ¹H NMR (DMSO-d₆, 400 MHz): δ 7.85 (dd, 1H, J=8.8, 10.3 Hz), 7.69 (d, 1H, J=8.3 Hz), 7.0-7.6 (m, 5H), 6.77 (d, 1H, J=8.8 Hz), 6.28 (br dd, 1H, J=6.4, 11.2 Hz), 5.62 (d, 1H, J=11.7 Hz), 4.99 (br d, 1H, J=14.2 Hz), 4.84 (br s, 1H), 3.85 (s, 3H), 3.5-3.8 (m, 2H), 2.82 (br s, 1H), 2.6-2.6 (m, 1H), 1.4-1.5 (m, 9H).

### Step G: 9-(Difluoromethoxy)-2,3-difluoro-10,11,12,13-tetrahydro-19H-5,18-methano-dibenzo[c,f]pyrido[3,2-j][1,5,9]triazacyclotridecine-15,19(14H)-dione hydrochloride Example 75

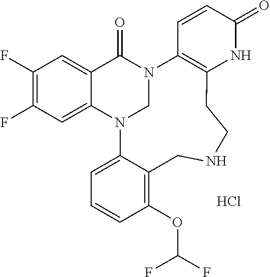 [SMILES: O=C1c2cc(F)c(F)cc2N2CN1c1ccc(=O)[nH]c1CCNCc1c(OC(F)F)cccc12 ; Cl]

Following the procedure outlined in Example 41, Step G, stirring the reaction mixture at 80° C. for 20 h, followed by addition of 5N HCl-ⁱPrOH (37 equiv) and stirring at 80° C. for 28 h, followed by cooling the reaction mixture and filtration, 9-(difluoromethoxy)-2,3-difluoro-10,11,12,13-tetrahydro-19H-5,18-methanodibenzo[c,f]pyrido-[3,2-j][1,5,9]triazacyclotridecine-15,19(14H)-dione hydrochloride (750 mg, 72% yield) was prepared as an off white solid. HPLC/MS 0.93 min (B), [M+H]⁺ 475.2. ¹H NMR (DMSO-d₆, 400 MHz): δ 2.64-2.80 (m, 1H) 2.88-3.07 (m, 2H) 3.20-3.35 (m, 1H) 4.00-4.17 (m, 2H) 4.96-5.05 (m, 1H) 5.36-5.55 (m, 2H) 6.16-6.26 (m, 1H) 6.31-6.38 (m, 1H) 7.14-7.56 (m, 4H) 7.67-7.74 (m, 1H) 7.82-7.92 (m, 1H) 8.71-8.87 (m, 1H) 10.18-10.33 (m, 1H).

### Example 76

### 8-Chloro-2,3-difluoro-10,11,12,13-tetrahydro-19H-5,18-methanodibenzo[c,f]pyrido[3,2-j][1,5,9]triazacyclotridecine-15,19(14H)-dione hydrochloride

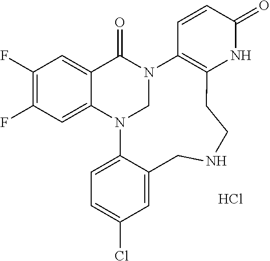 [SMILES: O=C1c2cc(F)c(F)cc2N2CN1c1ccc(=O)[nH]c1CCNCc1cc(Cl)ccc12 ; Cl]

### Step A: Methyl 2-((4-chloro-2-formylphenyl)amino)-4,5-difluorobenzoate 76a

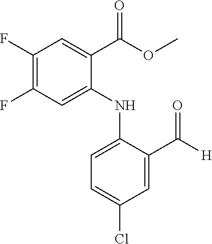 [SMILES: COC(=O)c1cc(F)c(F)cc1Nc1ccc(Cl)cc1C=O]

Following the preparation outlined in Example 7, Step A, substituting ethyl 2-bromo-5-(trifluoromethyl)benzoate with methyl 2-amino-4,5-difluorobenzoate, Int-3a with 2-bromo-5-chlorobenzaldehyde, BINAP with Xantphos, toluene with 1,4-dioxane, and stirring the reaction at 90° C. for 18 h, methyl 2-((4-chloro-2-formylphenyl)amino)-4,5-difluorobenzoate 76a (5.32 g, 72% yield) was prepared as a yellow solid. HPLC/MS 1.31 min (C), [M+H]⁺ 326.0. ¹H NMR (DMSO-d₆, 400 MHz): δ 11.00 (s, 1H), 9.96 (s, 1H), 7.97 (d, J=2.4 Hz, 1H), 7.93 (dd, J=11.2, 8.8 Hz, 1H), 7.63 (dd, J=12.7, 7.3 Hz, 1H), 7.56-7.60 (m, 1H), 7.51-7.54 (m, 1H), 3.87 (s, 3H).

### Step B: Methyl 2-((2-(((tert-butoxycarbonyl)(2-(6-methoxy-3-nitropyridin-2-yl)ethyl-amino)methyl)-4-chlorophenyl)amino)-4,5-difluorobenzoate 76b

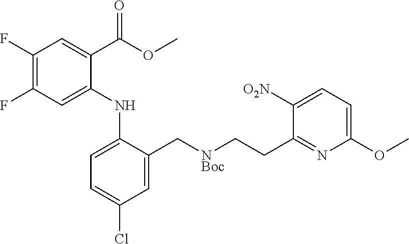 [SMILES: COC(=O)c1cc(F)c(F)cc1Nc1ccc(Cl)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((4-chloro-2-formylphenyl)amino)-4,5-difluorobenzoate 76a, methyl 2-((2-(((tert-butoxycarbonyl)(2-(6-methoxy-3-nitropyridin-2-yl)ethyl)amino)methyl)-4-chlorophenyl)amino)-4,5-difluorobenzoate 76b (4.19 g, 82% yield) was prepared as a white foam. HPLC/MS 1.66 min (A), [M+H]⁺ 607.2. ¹H NMR (DMSO-d₆, 400 MHz): δ 9.04 (s, 1H), 8.31 (d, J=9.3 Hz, 1H), 7.83 (dd, J=11.5, 9.0 Hz, 1H), 7.37-7.42 (m, 1H), 7.31-7.36 (m, 1H), 7.27 (br s, 1H), 6.85 (br d, J=7.8 Hz, 1H), 4.32 (br s, 2H), 3.88 (s, 3H), 3.87 (s, 3H), 3.63 (br s, 2H), 3.17 (br t, J=5.9 Hz, 2H), 1.22 (s, 9H).

### Step C: 2-((2-(((tert-Butoxycarbonyl)(2-(6-methoxy-3-nitropyridin-2-yl)ethyl)amino)-methyl)-4-chlorophenyl)amino)-4,5-difluorobenzoic acid hydrochloride 76c

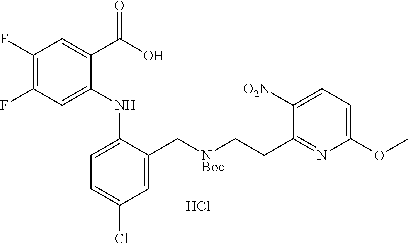 [SMILES: COc1ccc([N+](=O)[O-])c(CCN(Cc2cc(Cl)ccc2Nc2cc(F)c(F)cc2C(=O)O)C(=O)OC(C)(C)C)n1 ; Cl]

Following the procedure outlined in Example 41, Step C, stirring the reaction mixture at ambient temperature for 23 h, 2-((2-(((tert-butoxycarbonyl)(2-(6-methoxy-3-nitropyridin-2-yl)ethyl)amino)methyl)-4-chlorophenyl)amino)-4,5-difluorobenzoic acid hydrochloride 76c (4.23 g, 98% yield) was prepared as a foamy solid. HPLC/MS 1.53 min (C), [M+H]⁺ 493.1. ¹H NMR (DMSO-d₆, 400 MHz): δ 9.47 (br d, J=6.4 Hz, 1H), 8.31 (d, J=8.8 Hz, 1H), 7.81 (dd, J=11.7, 9.3 Hz, 1H), 7.36-7.40 (m, 1H), 7.31-7.36 (m, 1H), 7.23 (br s, 1H), 6.85 (br d, J=7.8 Hz, 1H), 6.38-6.65 (m, 1H), 4.34 (br d, J=1.0 Hz, 2H), 3.88 (s, 3H), 3.60-3.71 (m, 2H), 3.17 (br s, 2H), 1.20 (br d, J=2.0 Hz, 9H).

### Step D: 2-((2-(((2-(3-Amino-6-methoxypyridin-2-yl)ethyl)(tert-butoxycarbonyl)amino)-methyl)-4-chlorophenyl)amino)-4,5-difluorobenzoic acid 76d

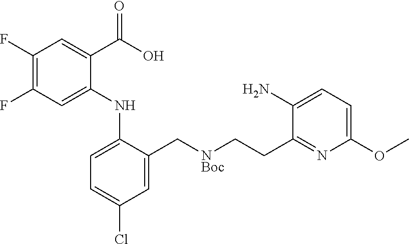 [SMILES: COc1ccc(N)c(CCN(Cc2cc(Cl)ccc2Nc2cc(F)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-4-chlorophenyl)amino)-4,5-difluorobenzoic acid 76d (3.44 g, 91% yield) as a foamy solid. HPLC/MS 1.10 min (A), [M+H]⁺ 563.1. ¹H NMR (DMSO-d₆, 400 MHz): δ 10.45-10.61 (m, 1H), 7.79 (br dd, J=11.0, 10.0 Hz, 1H), 7.27 (br s, 3H), 7.07 (s, 1H), 6.96 (d, J=8.3 Hz, 1H), 6.34 (d, J=8.8 Hz, 1H), 4.54 (br s, 2H), 4.35 (br s, 2H), 3.60 (s, 3H), 3.48 (br s, 2H), 2.71 (br t, J=7.1 Hz, 2H), 1.33 (br s, 9H).

### Step E: tert-Butyl 8-chloro-2,3-difluoro-15-methoxy-19-oxo-5,10,12,13,18,19-hexa-hydro-11H-dibenzo[c,f]pyrido[3,2-j][1,5,9]triazacyclotridecine-11-carboxylate 76e

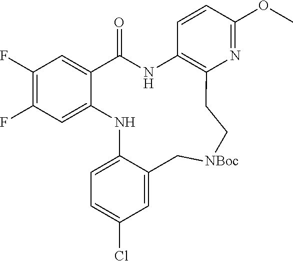 [SMILES: COc1ccc2c(n1)CCN(C(=O)OC(C)(C)C)Cc1cc(Cl)ccc1Nc1cc(F)c(F)cc1C(=O)N2]

Following the procedure outlined in Example 41, Step E, substituting HATU with pyoxim, and stirring the reaction mixture at room temperature for 20 h, tert-butyl 8-chloro-2,3-difluoro-15-methoxy-19-oxo-5,10,12,13,18,19-hexahydro-11H-dibenzo[c,f]pyrido[3,2-j][1,5,9]triazacyclo-tridecine-11-carboxylate 76e (583 mg, 18% yield) was prepared as a filmy solid. HPLC/MS 1.40 min (A), [M+H]⁺ 545.2. ¹H NMR (DMSO-d₆, 400 MHz): δ 10.22-10.33 (m, 1H), 7.85 (br d, J=8.8 Hz, 2H), 7.67 (s, 1H), 7.40 (d, J=2.4 Hz, 1H), 7.17 (dd, J=8.8, 2.4 Hz, 1H), 6.70 (d, J=8.8 Hz, 1H), 6.48-6.58 (m, 1H), 4.61 (s, 2H), 3.81 (s, 3H), 3.38 (br s, 2H), 2.52-2.61 (m, 2H), 1.38 (br s, 9H).

### Step F: tert-Butyl 8-chloro-2,3-difluoro-15-methoxy-19-oxo-12,13-dihydro-19H-5,18-methanodibenzo[c,f]pyrido[3,2-j][1,5,9]triazacyclotridecine-11(10H)-carboxylate 76f

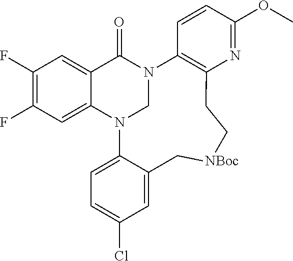 [SMILES: COc1ccc2c(n1)CCN(C(=O)OC(C)(C)C)Cc1cc(Cl)ccc1N1CN2C(=O)c2cc(F)c(F)cc21]

Following the procedure outlined in Example 41, Step F, stirring the reaction at 85° C. for 22 h, tert-butyl 8-chloro-2,3-difluoro-15-methoxy-19-oxo-12,13-dihydro-19H-5,18-methano-dibenzo[c,f]pyrido[3,2-j][1,5,9]triazacyclotridecine-11(10H)-carboxylate 76f (1.56 g, 84% yield) was prepared as a white solid. HPLC/MS 1.36 min (A), [M+H]⁺ 557.1. ¹H NMR (DMSO-d₆, 400 MHz): δ 7.85 (dd, J=10.8, 8.8 Hz, 1H), 7.71 (d, J=8.8 Hz, 1H), 7.56-7.60 (m, 1H), 7.52-7.55 (m, 1H), 7.44 (d, J=8.3 Hz, 1H), 6.78 (d, J=8.3 Hz, 1H), 6.30 (dd, J=11.7, 6.4 Hz, 1H), 5.60 (d, J=11.7 Hz, 1H), 4.89 (d, J=11.7 Hz, 1H), 4.71 (d, J=14.7 Hz, 1H), 3.89 (br d, J=14.7 Hz, 1H), 3.59-3.70 (m, 1H), 2.92 (td, J=11.4, 2.7 Hz, 1H), 2.62-2.74 (m, 1H), 2.56 (br d, J=4.9 Hz, 1H), 1.45 (s, 9H).

### Step G: 8-Chloro-2,3-difluoro-10,11,12,13-tetrahydro-19H-5,18-methanodibenzo-[c,f]pyrido[3,2-j][1,5,9]triazacyclotridecine-15,19(14H)-dione hydrochloride

### Example 76

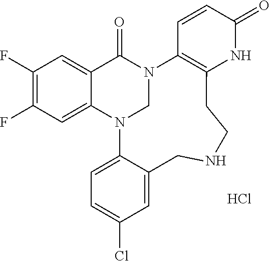 [SMILES: O=C1c2cc(F)c(F)cc2N2CN1c1ccc(=O)[nH]c1CCNCc1cc(Cl)ccc12 ; Cl]

Following the procedure outlined in Example 41, Step G, stirring the reaction mixture at 85° C. for 18 h, 8-chloro-2,3-difluoro-10,11,12,13-tetrahydro-19H-5,18-methanodibenzo-[c,f]pyrido[3,2-j][1,5,9]triazacyclotridecine-15,19(14H)-dione hydrochloride (295 mg, 89% yield) was prepared. HPLC/MS 0.63 min (A), [M+H]⁺ 443.1. ¹H NMR (DMSO-d₆, 400 MHz): δ 10.51 (br s, 1H), 8.96 (br s, 1H), 7.98 (d, J=2.45 Hz, 1H), 7.87 (dd, J=10.27, 8.80 Hz, 1H), 7.67-7.73 (m, 1H), 7.55 (d, J=8.31 Hz, 1H), 7.45 (d, J=9.78 Hz, 1H), 6.26-6.37 (m, 2H), 5.45 (d, J=11.74 Hz, 1H), 4.94 (d, J=11.25 Hz, 1H), 4.07-4.10 (m, 2H), 3.19 (br d, J=6.36 Hz, 1H), 2.90-3.04 (m, 2H), 2.62-2.72 (m, 1H).

### Example 77

### 8,9-Dichloro-2,3-difluoro-10,11,12,13-tetrahydro-19H-5,18-methanodibenzo[c,f]pyrido[3,2-j][1,5,9]triazacyclotridecine-15,19(14H)-dione hydrochloride

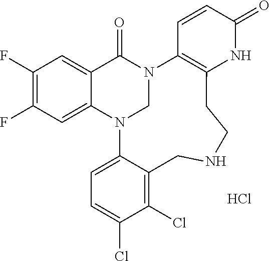 [SMILES: O=C1c2cc(F)c(F)cc2N2CN1c1ccc(=O)[nH]c1CCNCc1c2ccc(Cl)c1Cl ; Cl]

### Step A: tert-Butyl (6-bromo-2,3-dichlorobenzyl)(2-(6-methoxy-3-nitropyridin-2-yl)-ethyl)carbamate 77a

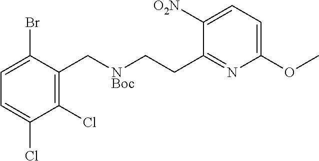 [SMILES: COc1ccc([N+](=O)[O-])c(CCN(Cc2c(Br)ccc(Cl)c2Cl)C(=O)OC(C)(C)C)n1]

Following the preparation outlined in Example 44, Step B, substituting methyl 5-fluoro-2-((4-fluoro-2-formylphenyl)amino)-4-(trifluoromethyl)benzoate with 6-bromo-2,3-dichloro-benzaldehyde, tert-butyl (6-bromo-2,3-dichlorobenzyl)(2-(6-methoxy-3-nitropyridin-2-yl)ethyl)carbamate 77a (7.73 g, 77% yield) was prepared as a thick clear yellow gel. HPLC/MS 1.58 min (B), [M+H]⁺ 534.0, 536.0. ¹H NMR (DMSO-d₆, 400 MHz): δ 8.32 (d, 1H, J=9.3 Hz), 7.66 (d, 1H, J=8.6 Hz), 7.55 (d, 1H, J=8.8 Hz), 6.87 (br d, 1H, J=8.8 Hz), 4.7-4.9 (m, 2H), 3.9-4.1 (m, 3H), 3.4-3.7 (m, 2H), 3.19 (br s, 2H), 1.2-1.3 (m, 9H).

### Step B: Methyl 2-((2-(((tert-butoxycarbonyl)(2-(6-methoxy-3-nitropyridin-2-yl)ethyl)-amino)methyl)-3,4-dichlorophenyl)amino)-4,5-difluorobenzoate 77b

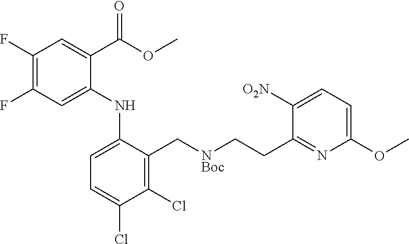 [SMILES: COC(=O)c1cc(F)c(F)cc1Nc1ccc(Cl)c(Cl)c1CN(CCc1nc(OC)ccc1[N+](=O)[O-])C(=O)OC(C)(C)C]

Following the preparation outlined in Example 7, Step A, substituting ethyl 2-bromo-5-(trifluoromethyl)benzoate with methyl 2-amino-4,5-difluorobenzoate, Int-3a with tert-butyl (6-bromo-2,3-dichlorobenzyl)(2-(6-methoxy-3-nitropyridin-2-yl)ethyl)carbamate Example 77a, BINAP with Xantphos, toluene with 1,4-dioxane, and stirring the reaction at 90° C. for 20 h, methyl 2-((2-(((tert-butoxycarbonyl)(2-(6-methoxy-3-nitropyridin-2-yl)ethyl)amino)methyl)-3,4-dichlorophenyl)amino)-4,5-difluorobenzoate 77b (3.55 g, 34% yield) was prepared as a yellow foam solid. HPLC/MS 1.71 min (B), [M+H]⁺ 641.0. ¹H NMR (DMSO-d₆, 400 MHz): δ 9.19 (s, 1H), 8.32 (br d, 1H, J=8.8 Hz), 7.79 (dd, 1H, J=8.8, 11.2 Hz), 7.62 (d, 1H, J=8.8 Hz), 7.34 (d, 1H, J=8.8 Hz), 6.86 (br d, 1H, J=8.8 Hz), 6.70 (br s, 1H), 4.67 (s, 2H), 3.9-3.9 (m, 3H), 3.84 (s, 3H), 3.5-3.5 (m, 2H), 3.13 (t, 2H, J=6.4 Hz), 1.1-1.2 (m, 9H).

### Step C: 2-((2-(((tert-Butoxycarbonyl)(2-(6-methoxy-3-nitropyridin-2-yl)ethyl)amino)-methyl)-3,4-dichlorophenyl)amino)-4,5-difluorobenzoic acid 77c

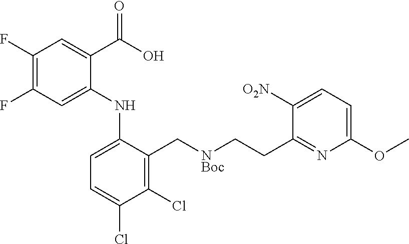 [SMILES: COc1ccc([N+](=O)[O-])c(CCN(Cc2c(Nc3cc(F)c(F)cc3C(=O)O)ccc(Cl)c2Cl)C(=O)OC(C)(C)C)n1]

Following the procedure outlined in Example 41, Step C, stirring the reaction mixture at 50° C. for 17 h, 2-((2-(((tert-butoxycarbonyl)(2-(6-methoxy-3-nitropyridin-2-yl)ethyl)amino)-methyl)-3,4-dichlorophenyl)amino)-4,5-difluorobenzoic acid 77c (3.58 g, 100% yield) was prepared as a foamy solid. HPLC/MS 1.14 min (B), [M+H]⁺ 627.0. ¹H NMR (DMSO-d₆, 400 MHz): δ 13.1-13.8 (m, 1H), 9.51 (br s, 1H), 8.31 (br d, 1H, J=8.8 Hz), 7.77 (dd, 1H, J=9.0, 11.5 Hz), 7.61 (d, 1H, J=8.8 Hz), 7.3-7.4 (m, 1H), 6.85 (br d, 1H, J=6.8 Hz), 6.67 (br s, 1H), 4.67 (s, 2H), 3.90 (br s, 3H), 3.5-3.6 (m, 2H), 3.13 (t, 2H, J=6.4 Hz), 1.1-1.2 (m, 9H).

### Step D: 2-((2-(((2-(3-Amino-6-methoxypyridin-2-yl)ethyl)(tert-butoxycarbonyl)amino)-methyl)-3,4-dichlorophenyl)amino)-4,5-difluorobenzoic acid 77d

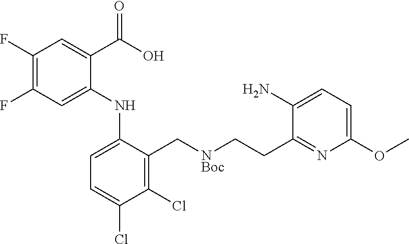 [SMILES: COc1ccc(N)c(CCN(Cc2c(Nc3cc(F)c(F)cc3C(=O)O)ccc(Cl)c2Cl)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3,4-dichlorophenyl)amino)-4,5-difluorobenzoic acid 77d (3.33 g, 98% yield) as a light yellow solid. HPLC/MS 1.06 min (B), [M+H]⁺ 597.2. ¹H NMR (DMSO-d₆, 400 MHz): δ 10.56 (br s, 1H), 7.74 (dd, 1H, J=9.5, 11.5 Hz), 7.52 (d, 1H, J=8.8 Hz), 7.28 (d, 1H, J=8.8 Hz), 6.93 (d, 1H, J=8.3 Hz), 6.56 (dd, 1H, J=6.8, 13.2 Hz), 6.31 (d, 1H, J=8.3 Hz), 4.62 (br s, 2H), 4.46 (br s, 2H), 3.58 (s, 3H), 3.2-3.3 (m, 2H), 2.5-2.6 (m, 2H), 1.2-1.3 (m, 9H) (one proton hidden).

### Step E: tert-Butyl 8,9-dichloro-2,3-difluoro-15-methoxy-19-oxo-5,10,12,13,18,19-hexa-hydro-11H-dibenzo[c,f]pyrido[3,2-j][1,5,9]triazacyclotridecine-11-carboxylate 77e

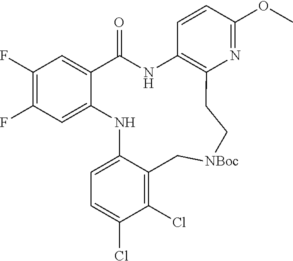 [SMILES: COc1ccc2c(n1)CCN(C(=O)OC(C)(C)C)Cc1c(ccc(Cl)c1Cl)Nc1cc(F)c(F)cc1C(=O)N2]

Following the procedure outlined in Example 41, Step E, stirring the reaction mixture at RT for 2 h, tert-butyl 8,9-dichloro-2,3-difluoro-15-methoxy-19-oxo-5,10,12,13,18,19-hexahydro-11H-dibenzo[c,f]pyrido[3,2-j][1,5,9]triazacyclotridecine-11-carboxylate 77e (1.79 g, 60% yield) was prepared as a light yellow solid. HPLC/MS 1.53 min (B), [M+H]⁺ 579.1. ¹H NMR (DMSO-d₆, 400 MHz): δ 10.08 (s, 1H), 7.8-7.9 (m, 3H), 7.40 (br s, 1H), 7.31 (d, 1H, J=9.3 Hz), 6.68 (d, 1H, J=8.3 Hz), 6.36 (d, 1H, J=8.8 Hz), 4.83 (br s, 2H), 3.81 (s, 3H), 2.6-3.2 (m, 4H), 1.46 (br s, 9H).

### Step F: tert-Butyl 8,9-dichloro-2,3-difluoro-15-methoxy-19-oxo-12,13-dihydro-19H-5,18-methanodibenzo[c,f]pyrido[3,2-j][1,5,9]triazacyclotridecine-11(10H)-carboxylate 77f

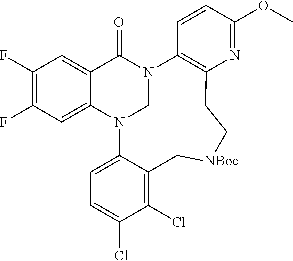 [SMILES: COc1ccc2c(n1)CCN(C(=O)OC(C)(C)C)Cc1c(ccc(Cl)c1Cl)N1CN2C(=O)c2cc(F)c(F)cc21]

Following the procedure outlined in Example 41, Step F, stirring the reaction at 90° C. for 17 h, tert-butyl 8,9-dichloro-2,3-difluoro-15-methoxy-19-oxo-12,13-dihydro-19H-5,18-methano-dibenzo[c,f]pyrido[3,2-j][1,5,9]triazacyclotridecine-11(10H)-carboxylate 77f (1.30 g, 70% yield) was prepared as an off white solid. HPLC/MS 1.48 min (B), [M+H]⁺ 591.1. ¹H NMR (DMSO-d₆, 400 MHz): δ 7.8-7.9 (m, 2H), 7.68 (d, 1H, J=8.3 Hz), 7.43 (d, 1H, J=8.8 Hz), 6.77 (d, 1H, J=8.3 Hz), 6.50 (br dd, 1H, J=7.1, 12.0 Hz), 5.61 (d, 1H, J=11.7 Hz), 4.8-5.0 (m, 2H), 3.9-4.2 (m, 1H), 3.84 (s, 3H), 3.5-3.8 (m, 1H), 2.7-2.9 (m, 1H), 2.6-2.6 (m, 2H), 1.43 (br s, 9H).

### Step G: 8,9-Chloro-2,3-difluoro-10,11,12,13-tetrahydro-19H-5,18-methanodibenzo-[c,f]pyrido[3,2-j][1,5,9]triazacyclotridecine-15,19(14H)-dione hydrochloride

### Example 77

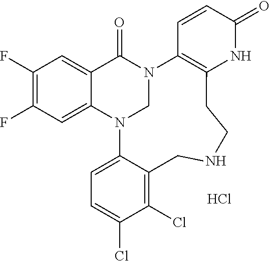 [SMILES: O=C1c2cc(F)c(F)cc2N2CN1c1ccc(=O)[nH]c1CCNCc1c2ccc(Cl)c1Cl ; Cl]

Following the procedure outlined in Example 41, Step G, stirring the reaction mixture at 80° C. for 12 h, 8,9-dichloro-2,3-difluoro-10,11,12,13-tetrahydro-19H-5,18-methanodibenzo-[c,f]pyrido[3,2-j][1,5,9]triazacyclotridecine-15,19(14H)-dione hydrochloride (295 mg, 89% yield) was prepared as a white solid. HPLC/MS 1.03 min (B), [M+H]⁺ 477.1. ¹H NMR (DMSO-d₆, 400 MHz): δ 10.43 (br s, 1H), 8.78 (br s, 1H), 7.9-8.0 (m, 1H, J=8.3 Hz), 7.8-7.9 (m, 1H), 7.6-7.6 (m, 1H, J=8.3 Hz), 7.46 (d, 1H, J=9.3 Hz), 6.42 (dd, 1H, J=6.6, 12.0 Hz), 6.34 (d, 1H, J=9.3 Hz), 5.43 (d, 2H, J=11.7 Hz), 5.03 (d, 1H, J=11.2 Hz), 4.2-4.3 (m, 2H), 3.2-3.4 (m, 1H), 3.0-3.1 (m, 2H), 2.7-2.8 (m, 1H).

### Example 78

### 2-(Trifluoromethyl)-10,11,12,13-tetrahydro-19H-5,18-methanodibenzo[c,f]pyrido[3,2-j][1,5,9]triazacyclotridecine-15,19(14H)-dione hydrochloride

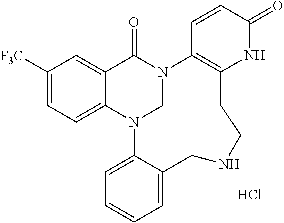 [SMILES: O=C1c2cc(C(F)(F)F)ccc2N2CN1c1ccc(=O)[nH]c1CCNCc1ccccc12 ; Cl]

### Step A: Methyl 2-((2-formylphenyl)amino)-5-(trifluoromethyl)benzoate 78a

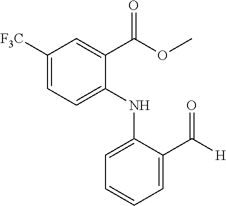 [SMILES: COC(=O)c1cc(C(F)(F)F)ccc1Nc1ccccc1C=O]

Following the preparation outlined in Example 7, Step A, substituting ethyl 2-bromo-5-(trifluoromethyl)benzoate with methyl 2-amino-5-(trifluoromethyl)benzoate, Int-3a with 2-bromobenzaldehyde, BINAP with Xantphos, toluene with 1,4-dioxane, and stirring the reaction at 80° C. for 8 h, methyl 2-((2-formylphenyl)amino)-5-(trifluoromethyl)benzoate 78a (3.55 g, 79% yield) was prepared as a yellow solid. HPLC/MS 1.31 min (B), [M+H]⁺ 324.1. ¹H NMR (CDCl₃, 400 MHz): δ 11.45 (s, 1H), 10.03 (s, 1H), 8.29 (d, J=2.0 Hz, 1H), 7.73 (dd, J=7.8, 1.5 Hz, 1H), 7.54-7.67 (m, 3H), 7.47-7.52 (m, 1H), 7.13 (t, J=7.4 Hz, 1H), 3.99 (s, 3H).

### Step B: Methyl 2-((2-(((tert-butoxycarbonyl)(2-(6-methoxy-3-nitropyridin-2-yl)ethyl-amino)methyl)phenyl)amino)-5-(trifluoromethyl)benzoate 78b

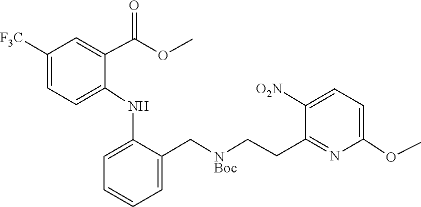 [SMILES: COC(=O)c1cc(C(F)(F)F)ccc1Nc1ccccc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((2-formylphenyl)-amino)-5-(trifluoromethyl)benzoate 78a, methyl 2-((2-(((tert-butoxycarbonyl)(2-(6-methoxy-3-nitropyridin-2-yl)ethyl)amino)methyl)phenyl)amino)-5-(trifluoromethyl)benzoate 78b (3.97 g, 56% yield) was prepared as a clear oil. HPLC/MS 1.67 min (B), [M+H]⁺ 605.3. ¹H NMR (CDCl₃, 400 MHz): δ 9.55 (s, 1H), 8.20 (s, 1H), 8.13 (d, J=8.8 Hz, 1H), 7.38 (dd, J=8.8, 2.0 Hz, 2H), 7.23-7.31 (m, 3H), 6.69 (br s, 1H), 6.58 (br d, J=8.8 Hz, 1H), 4.48 (br s, 2H), 3.93 (s, 3H), 3.89 (s, 3H), 3.59-3.76 (m, 2H), 3.21-3.31 (m, 2H), 1.35 (s, 9H).

### Step C: 2-((2-(((tert-Butoxycarbonyl)(2-(6-methoxy-3-nitropyridin-2-yl)ethyl)amino)-methyl)phenyl)amino)-5-(trifluoromethyl)benzoic acid 78c

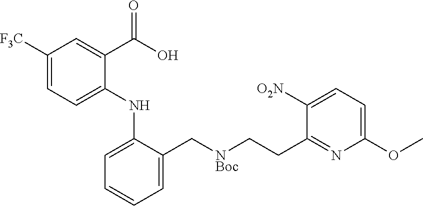 [SMILES: COc1ccc([N+](=O)[O-])c(CCN(Cc2ccccc2Nc2ccc(C(F)(F)F)cc2C(=O)O)C(=O)OC(C)(C)C)n1]

Following the procedure outlined in Example 41, Step C, stirring the reaction mixture at room temperature overnight, 2-((2-(((tert-butoxycarbonyl)(2-(6-methoxy-3-nitropyridin-2-yl)ethyl)amino)methyl)phenyl)amino)-5-(trifluoromethyl)benzoic acid 78c (3.71 g, 91% yield) was prepared as a yellow powder. HPLC/MS 1.11 min (B), [M+H]⁺ 591.3. ¹H NMR (CDCl₃, 400 MHz): δ 9.53 (br s, 1H), 8.11-8.26 (m, 2H), 7.44 (br d, J=9.0 Hz, 1H), 7.29-7.38 (m, 4H), 6.72 (br d, J=8.5 Hz, 1H), 6.64 (br d, J=9.0 Hz, 1H), 4.56 (br s, 2H), 3.92 (s, 3H), 3.69 (br s, 2H), 3.31 (br s, 2H), 1.40 (s, 9H).

### Step D: 2-((2-(((2-(3-Amino-6-methoxypyridin-2-yl)ethyl)(tert-butoxycarbonyl)amino)-methyl)phenyl)amino)-5-(trifluoromethyl)benzoic acid 78d

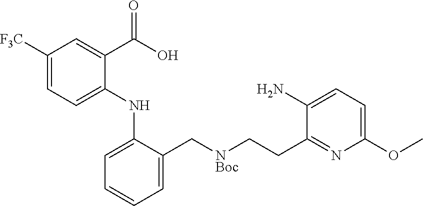 [SMILES: COc1ccc(N)c(CCN(Cc2ccccc2Nc2ccc(C(F)(F)F)cc2C(=O)O)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)phenyl)amino)-5-(trifluoro-methyl)benzoic acid 78d (3.40 g, 93% yield) as a grayish brown solid. HPLC/MS 1.00 min (B), [M+H]⁺ 561.3. ¹H NMR (CDCl₃, 400 MHz): δ 9.60 (br s, 1H), 8.23 (br s, 1H), 7.23-7.41 (m, 5H), 7.00 (br d, J=8.8 Hz, 1H), 6.74 (br d, J=8.8 Hz, 1H), 6.42 (d, J=8.8 Hz, 1H), 4.45 (br s, 2H), 3.74 (s, 3H), 3.42-3.54 (m, 2H), 2.86 (br s, 2H), 1.42 (br s, 9H).

### Step E: tert-Butyl 15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 78e

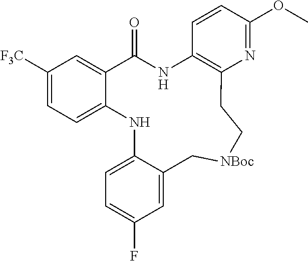 [SMILES: COc1ccc2c(n1)CCN(C(=O)OC(C)(C)C)Cc1cc(F)ccc1Nc1ccc(C(F)(F)F)cc1C(=O)N2]

Following the procedure outlined in Example 41, Step E, stirring the reaction mixture at room temperature for 3 h, tert-butyl 15-methoxy-19-oxo-2-(trifluoromethyl)-5,10,12,13,18,19-hexahydro-11H-dibenzo[c,f]pyrido[3,2-j][1,5,9]triazacyclotridecine-11-carboxylate 78e (2.55 g, 70% yield) was prepared as a white solid. HPLC/MS 1.44 min (B), [M+H]⁺ 543.3. ¹H NMR (CDCl₃, 400 MHz): δ 10.61-10.80 (m, 1H) 8.70 (s, 1H) 8.21 (d, J=9.0 Hz, 1H) 7.85 (dd, J=8.0, 2.0 Hz, 1H) 7.42 (d, J=8.5 Hz, 1H) 7.26-7.28 (m, 1H) 7.18 (td, J=7.8, 1.5 Hz, 1H) 6.98-6.99 (m, 1H) 6.91-7.00 (m, 1H) 6.62 (d, J=9.0 Hz, 1H) 6.52 (s, 1H) 6.41 (br d, J=8.0 Hz, 1H) 3.81-3.92 (m, 4H) 3.47-3.80 (m, 2H) 2.29 (br s, 2H) 1.10 (s, 9H).

### Step F: tert-Butyl 15-methoxy-19-oxo-2-(trifluoromethyl)-12,13-dihydro-19H-5,18-methanodibenzo[c,f]pyrido[3,2-j][1,5,9]triazacyclotridecine-11(10H)-carboxylate 78f

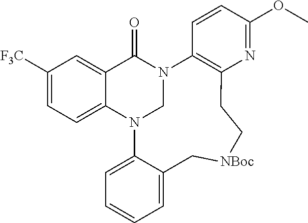 [SMILES: COc1ccc2c(n1)CCN(C(=O)OC(C)(C)C)Cc1ccccc1N1CN2C(=O)c2cc(C(F)(F)F)ccc21]

Following the procedure outlined in Example 41, Step F, stirring the reaction at 90° C. overnight, tert-butyl 15-methoxy-19-oxo-2-(trifluoromethyl)-12,13-dihydro-19H-5,18-methano-dibenzo[c,]pyrido[3,2-j][1,5,9]triazacyclotridecine-11(10H)-carboxylate 78f (482 mg, 18% yield) was prepared as a light tan solid. HPLC/MS 1.44 min (B), [M+H]⁺ 555.3. ¹H NMR (CDCl₃, 400 MHz): δ 8.38 (d, J=1.5 Hz, 1H), 7.60-7.75 (m, 1H), 7.38-7.54 (m, 4H), 7.07-7.25 (m, 1H), 6.65 (d, J=8.3 Hz, 1H), 6.39 (d, J=8.8 Hz, 1H), 5.63 (d, J=11.2 Hz, 1H), 4.85 (br d, J=14.7 Hz, 1H), 4.62 (d, J=11.2 Hz, 1H), 4.03 (br d, J=14.7 Hz, 1H), 3.82-3.96 (m, 4H), 3.07 (td, J=11.7, 2.9 Hz, 1H), 2.72-2.85 (m, 1H), 2.56 (br s, 1H), 1.52 (s, 9H).

### Step G: 2-(Trifluoromethyl)-10,11,12,13-tetrahydro-19H-5,18-methanodibenzo[c,f]-pyrido[3,2-j][1,5,9]triazacyclotridecine-15,19(14H)-dione hydrochloride Example 78

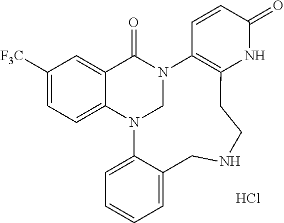 [SMILES: O=C1c2cc(C(F)(F)F)ccc2N2CN1c1ccc(=O)[nH]c1CCNCc1ccccc12 ; Cl]

Following the procedure outlined in Example 41, Step G, stirring the reaction mixture at 90° C. overnight, 2-(trifluoromethyl)-10,11,12,13-tetrahydro-19H-5,18-methanodibenzo-[c,]pyrido[3,2-j][1,5,9]triazacyclotridecine-15,19(14H)-dione hydrochloride (350 mg, 84% yield) was prepared as a white solid. HPLC/MS 0.98 min (B), [M+H]⁺ 441.1. ¹H NMR (DMSO-d₆, 400 MHz): δ 10.67 (br s, 1H), 8.72-8.96 (m, 1H), 8.13 (d, J=2.0 Hz, 1H), 7.92 (dd, J=7.8, 1.5 Hz, 1H), 7.50-7.72 (m, 4H), 7.46 (d, J=9.3 Hz, 1H), 6.32-6.42 (m, 2H), 5.52 (d, J=11.7 Hz, 1H), 5.02 (d, J=11.2 Hz, 1H), 4.07-4.18 (m, 1H), 3.97-4.07 (m, 1H), 3.13-3.21 (m, 1H), 2.93-3.12 (m, 2H), 2.57-2.74 (m, 1H).

### Example 79

### 9-(Difluoromethyl)-2,3-difluoro-10,11,12,13-tetrahydro-19H-5,18-methanodibenzo[c,f]pyrido[3,2-j][1,5,9]triazacyclotridecine-15,19(14H)-dione hydrochloride

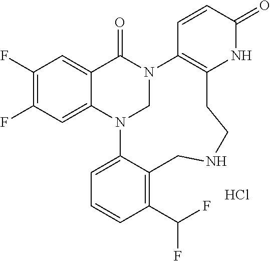 [SMILES: O=C1c2cc(F)c(F)cc2N2CN1c1ccc(=O)[nH]c1CCNCc1c(C(F)F)cccc12 ; Cl]

### Step A: Methyl 2-((3-(difluoromethyl)-2-formylphenyl)amino)-4,5-difluorobenzoate 79a

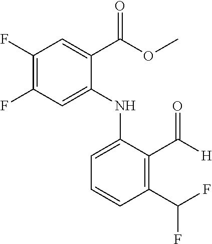 [SMILES: COC(=O)c1cc(F)c(F)cc1Nc1cccc(C(F)F)c1C=O]

Following the preparation outlined in Example 7, Step A, substituting ethyl 2-bromo-5-(trifluoromethyl)benzoate with methyl 2-amino-4,5-difluorobenzoate, Int-3a with 2-bromo-6-(difluoromethyl)benzaldehyde, BINAP with Xantphos, toluene with 1,4-dioxane, and stirring the reaction at 90° C. for 6 h, methyl 2-((3-(difluoromethyl)-2-formylphenyl)amino)-4,5-difluorobenzoate 79a (1.17 g, 80% yield) was prepared as a yellow solid. HPLC/MS 1.22 min (A), [M+H]⁺ 342.0. ¹H NMR (CDCl₃, 400 MHz): δ 11.47 (s, 1H), 10.51 (t, 1H, J=1.5 Hz), 7.88 (dd, 1H, J=9.0, 11.0 Hz), 7.5-7.7 (m, 2H), 7.1-7.3 (m, 3H), 7.03 (s, 1H), 6.90 (s, 1H), 3.98 (s, 3H).

### Step B: Methyl 2-((2-(((tert-butoxycarbonyl)(2-(6-methoxy-3-nitropyridin-2-yl)ethyl-amino)methyl)-3-(difluoromethyl)phenyl)amino)-4,5-difluorobenzoate 79b

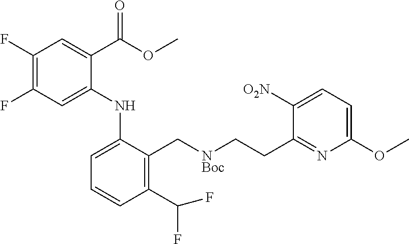 [SMILES: COC(=O)c1cc(F)c(F)cc1Nc1cccc(C(F)F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3-(difluoromethyl)-2-formylphenyl)amino)-4,5-difluorobenzoate 79a, methyl 2-((2-(((tert-butoxycarbonyl)(2-(6-methoxy-3-nitropyridin-2-yl)ethyl)amino)methyl)-3-(difluoromethyl)-phenyl)amino)-4,5-difluorobenzoate 79b (1.86 g, 89% yield) was prepared as a yellow foam. HPLC/MS 1.58 min (A), [M+H]⁺ 623.2. ¹H NMR (CDCl₃, 400 MHz): δ 9.41 (s, 1H), 8.20 (d, 1H, J=9.3 Hz), 7.7-7.8 (m, 1H), 7.63 (d, 1H, J=7.8 Hz), 7.5-7.5 (m, 1H), 7.4-7.4 (m, 1H), 7.0-7.2 (m, 1H), 7.13 (br t, 1H, J=54.8 Hz), 6.6-6.7 (m, 1H), 6.41 (dd, 1H, J=6.8, 12.7 Hz), 4.77 (s, 2H), 3.91 (d, 6H, J=5.4 Hz), 3.5-3.6 (m, 2H), 3.19 (br t, 2H, J=7.1 Hz), 1.37 (s, 9H).

### Step C: 2-((2-(((tert-Butoxycarbonyl)(2-(6-methoxy-3-nitropyridin-2-yl)ethyl)amino)-methyl)-3-(difluoromethyl)phenyl)amino)-4,5-difluorobenzoic acid 79c

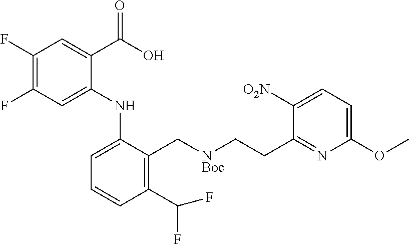 [SMILES: COc1ccc([N+](=O)[O-])c(CCN(Cc2c(Nc3cc(F)c(F)cc3C(=O)O)cccc2C(F)F)C(=O)OC(C)(C)C)n1]

Following the procedure outlined in Example 41, Step C, stirring the reaction mixture at 50° C. for 22 h, 2-((2-(((tert-butoxycarbonyl)(2-(6-methoxy-3-nitropyridin-2-yl)ethyl)amino)-methyl)-3-(difluoromethyl)phenyl)amino)-4,5-difluorobenzoic acid 79c (1.86 g, 100% yield) was prepared as an off white foam. HPLC/MS 1.47 min (A), [M+H]⁺ 609.1. ¹H NMR (DMSO-d₆, 400 MHz): δ 13.43 (br s, 1H), 9.57 (s, 1H), 8.30 (d, 1H, J=8.8 Hz), 7.7-7.8 (m, 1H), 7.5-7.6 (m, 3H), 7.0-7.4 (m, 1H), 6.8-6.9 (m, 1H), 6.4-6.6 (m, 1H), 4.65 (s, 2H), 3.4-3.5 (m, 2H), 3.33 (s, 3H), 3.0-3.2 (m, 2H), 1.1-1.3 (m, 10H).

### Step D: 2-((2-(((2-(3-Amino-6-methoxypyridin-2-yl)ethyl)(tert-butoxycarbonyl)amino) methyl)-3-(difluoromethyl)phenyl)amino)-4,5-difluorobenzoic acid 79d

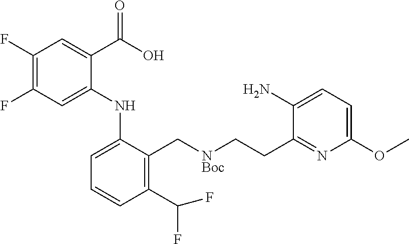 [SMILES: COc1ccc(N)c(CCN(Cc2c(Nc3cc(F)c(F)cc3C(=O)O)cccc2C(F)F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3-(difluoromethyl)phenyl)-amino)-4,5-difluorobenzoic acid 79d (1.23 g, 66% yield) as a light brown foam. HPLC/MS 1.08 min (B), [M+H]⁺ 579.1. ¹H NMR (DMSO-d₆, 400 MHz): δ 9.4-9.6 (m, 1H), 7.7-7.8 (m, 1H), 7.5-7.6 (m, 3H), 7.1-7.4 (m, 2H), 6.9-7.0 (m, 1H), 6.4-6.5 (m, 1H), 6.3-6.4 (m, 1H), 4.58 (s, 2H), 3.61 (s, 3H), 3.1-3.4 (m, 2H), 2.6-2.6 (m, 2H), 1.2-1.3 (m, 10H).

### Step E: tert-Butyl 9-(difluoromethyl)-2,3-difluoro-15-methoxy-19-oxo-5,10,12,13,18,19-hexahydro-11H-dibenzo-[c,f]pyrido[3,2-j][1,5,9]triazacyclotridecine-11-carboxylate 79e

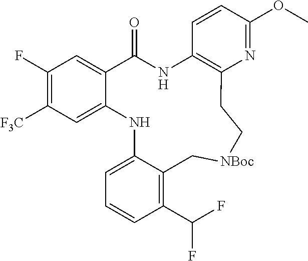 [SMILES: COc1ccc2c(n1)CCN(C(=O)OC(C)(C)C)Cc1c(cccc1C(F)F)Nc1cc(C(F)(F)F)c(F)cc1C(=O)N2]

Following the procedure outlined in Example 41, Step E, stirring the reaction mixture at room temperature for 48 h, tert-butyl 9-(difluoromethyl)-2,3-difluoro-15-methoxy-19-oxo-5,10,12,13,18,19-hexahydro-11H-dibenzo[c,f]pyrido[3,2-j][1,5,9]triazacyclotridecine-11-carboxylate 79e (642 mg, 52% yield) was prepared as a white solid. HPLC/MS 1.38 min (B), [M+H]⁺ 561.2. ¹H NMR (DMSO-d₆, 400 MHz): δ 10.18 (s, 1H), 7.8-8.0 (m, 2H), 7.75 (br s, 1H), 7.3-7.4 (m, 1H), 7.2-7.3 (m, 1H), 7.14 (d, 1H, J=7.8 Hz), 6.9-7.1 (m, 1H), 6.69 (d, 1H, J=8.8 Hz), 6.56 (d, 1H, J=7.8 Hz), 4.7-5.1 (m, 2H), 3.80 (s, 3H), 2.7-3.2 (m, 4H), 1.46 (s, 10H).

### Step F: tert-Butyl 9-(difluoromethyl)-2,3-difluoro-15-methoxy-19-oxo-12,13-dihydro-19H-5,18-methanodibenzo[c,f]pyrido[3,2-j][1,5,9]triazacyclotridecine-11(10H)-carboxylate 79f

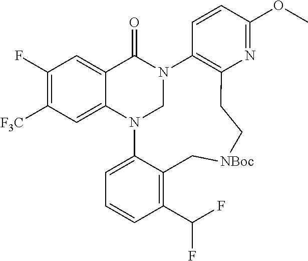 [SMILES: COc1ccc2c(n1)CCN(C(=O)OC(C)(C)C)Cc1c(C(F)F)cccc1N1CN2C(=O)c2cc(F)c(C(F)(F)F)cc21]

Following the procedure outlined in Example 41, Step F, stirring the reaction at 90° C. overnight, tert-butyl 9-(difluoromethyl)-2,3-difluoro-15-methoxy-19-oxo-12,13-dihydro-19H-5,18-methanodibenzo[c,f]pyrido[3,2-j][1,5,9]triazacyclotridecine-11(10H)-carboxylate 79f (429.1 mg, 64% yield) was prepared as an off white solid. HPLC/MS 1.38 min (A), [M+H]⁺ 573.1. ¹H NMR (DMSO-d₆, 400 MHz): δ 7.86 (br d, 1H, J=1.5 Hz), 7.75 (s, 1H), 7.6-7.7 (m, 2H), 7.59 (d, 1H, J=7.8 Hz), 7.21 (br t, 1H, J=54.5 Hz), 6.77 (d, 1H, J=8.3 Hz), 6.1-6.3 (m, 1H), 5.65 (d, 1H, J=11.7 Hz), 5.01 (br d, 1H, J=15.2 Hz), 4.8-5.0 (m, 1H), 4.0-4.3 (m, 1H), 3.84 (s, 3H), 3.4-3.7 (m, 1H), 2.8-3.0 (m, 1H), 2.6-2.7 (m, 1H), 1.45 (br s, 10H).

### Step G: 9-(Difluoromethyl)-2,3-difluoro-10,11,12,13-tetrahydro-19H-5,18-methano-dibenzo[c,f]pyrido[3,2-j][1,5,9]triazacyclotridecine-15,19(14H)-dione hydrochloride

### Example 79

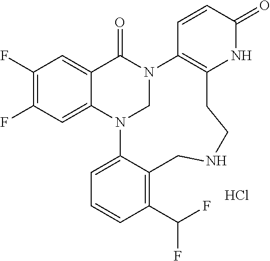 [SMILES: O=C1c2cc(F)c(F)cc2N2CN1c1ccc(=O)[nH]c1CCNCc1c(C(F)F)cccc12 ; Cl]

Following the procedure outlined in Example 41, Step G, stirring the reaction mixture at 90° C. 24 h, 9-(difluoromethyl)-2,3-difluoro-10,11,12,13-tetrahydro-19H-5,18-methanodibenzo-[c,f]pyrido[3,2-j][1,5,9]triazacyclotridecine-15,19(14H)-dione hydrochloride (316.2 mg, 83% yield) was prepared as a white solid. HPLC/MS 0.57 min (A), [M+H]⁺ 459.1. ¹H NMR (CD₃OD, 400 MHz): δ 7.8-8.0 (m, 4H), 7.7-7.8 (m, 1H), 7.29 (t, 1H, J=54.5 Hz), 6.79 (d, 1H, J=9.3 Hz), 6.10 (dd, 1H, J=6.6, 11.5 Hz), 5.63 (d, 1H, J=11.2 Hz), 5.07 (d, 1H, J=11.2 Hz), 4.55 (dd, 2H, J=14.2, 63.6 Hz), 3.3-3.6 (m, 2H), 2.9-3.0 (m, 1H).

### Example 80

### 2,9-Difluoro-3-(trifluoromethyl)-10,11,12,13-tetrahydro-19H-5,18-methanodibenzo-[c,f]pyrido[3,2-j][1,5,9]triazacyclotridecine-15,19(14H)-dione hydrochloride

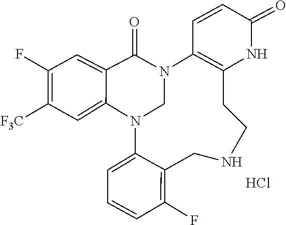 [SMILES: O=C1c2cc(F)c(C(F)(F)F)cc2N2CN1c1ccc(=O)[nH]c1CCNCc1c(F)cccc12 ; Cl]

### Step A: Methyl 5-fluoro-2-((3-fluoro-2-formylphenyl)amino)-4-(trifluoromethyl)-benzoate 80a

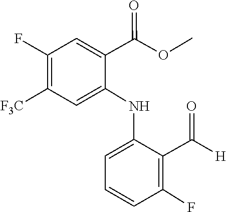 [SMILES: COC(=O)c1cc(F)c(C(F)(F)F)cc1Nc1cccc(F)c1C=O]

Following the preparation outlined in Example 7, Step A, substituting ethyl 2-bromo-5-(trifluoromethyl)benzoate with methyl 2-bromo-5-fluoro-4-(trifluoromethyl)benzoate, Int-3a with 2-amino-6-fluorobenzaldehyde, BINAP with Xantphos, toluene with 1,4-dioxane, and stirring the reaction at 100° C. overnight, methyl 5-fluoro-2-((3-fluoro-2-formylphenyl)amino)-4-(trifluoromethyl)benzoate 80a (2.06 g, 56% yield) was prepared as a yellow solid. HPLC/MS 1.35 min (B), [M+H]⁺ 360.1. ¹H NMR (CDCl₃, 400 MHz): δ 11.48 (br s, 1H), 10.46 (s, 1H), 7.86 (br d, J=8.5 Hz, 2H), 7.33-7.53 (m, 1H), 7.12 (d, J=8.5 Hz, 1H), 6.66 (dd, J=10.5, 8.0 Hz, 1H), 4.02 (s, 3H).

### Step B: Methyl 2-((2-(((tert-butoxycarbonyl)(2-(6-methoxy-3-nitropyridin-2-yl)ethyl-amino)methyl)-3-fluorophenyl)amino)-5-fluoro-4-(trifluoromethyl)benzoate 80b

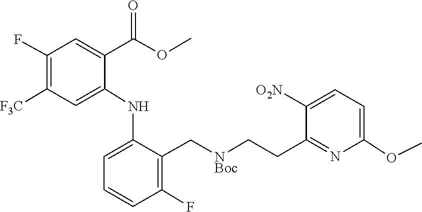 [SMILES: COC(=O)c1cc(F)c(C(F)(F)F)cc1Nc1cccc(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 5-fluoro-2-((3-fluoro-2-formylphenyl)amino)-4-(trifluoromethyl)benzoate 80a, methyl 2-((2-(((tert-butoxy-carbonyl)(2-(6-methoxy-3-nitropyridin-2-yl)ethyl)amino)methyl)-3-fluorophenyl)amino)-5-fluoro-4-(trifluoromethyl)benzoate 80b (2.04 g, 55% yield) was prepared as a light yellow oil. HPLC/MS 1.68 min (B), [M+H]⁺ 641.3. ¹H NMR (CDCl₃, 400 MHz): δ 9.20 (br s, 1H), 8.22 (d, J=9.0 Hz, 1H), 7.75 (d, J=10.5 Hz, 1H), 7.26-7.31 (m, 1H), 7.15 (br d, J=5.5 Hz, 1H), 7.08 (d, J=8.0 Hz, 1H), 6.90 (t, J=8.9 Hz, 1H), 6.66 (br d, J=9.0 Hz, 1H), 4.65 (s, 2H), 3.98 (s, 3H), 3.93 (s, 3H), 3.68 (t, J=7.0 Hz, 2H), 3.31 (t, J=6.8 Hz, 2H), 1.31 (s, 9H).

### Step C: 2-((2-(((tert-Butoxycarbonyl)(2-(6-methoxy-3-nitropyridin-2-yl)ethyl)amino)-methyl)-3-fluorophenyl)amino)-5-fluoro-4-(trifluoromethyl)benzoic acid 80c

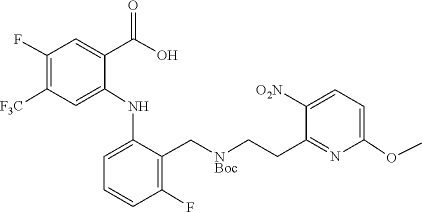 [SMILES: COc1ccc([N+](=O)[O-])c(CCN(Cc2c(F)cccc2Nc2cc(C(F)(F)F)c(F)cc2C(=O)O)C(=O)OC(C)(C)C)n1]

Following the procedure outlined in Example 41, Step C, stirring the reaction mixture at room temperature overnight, 2-((2-(((tert-butoxycarbonyl)(2-(6-methoxy-3-nitropyridin-2-yl)ethyl)amino)methyl)-3-fluorophenyl)amino)-5-fluoro-4-(trifluoromethyl)benzoic acid 80c (2.12 g, 100% yield) was prepared as a yellow powder. HPLC/MS 1.14 min (B), [M+H]⁺ 627.3. ¹H NMR (CDCl₃, 400 MHz): δ 9.26 (br s, 1H), 8.25 (d, J=9.0 Hz, 1H), 7.82 (d, J=10.5 Hz, 1H), 7.18-7.41 (m, 2H), 7.07 (br d, J=8.0 Hz, 1H), 6.94 (t, J=8.9 Hz, 1H), 6.69 (d, J=9.0 Hz, 1H), 4.64 (s, 2H), 4.01 (s, 3H), 3.71 (t, J=6.8 Hz, 2H), 3.34 (t, J=6.8 Hz, 2H), 1.23-1.33 (m, 9H).

### Step D: 2-((2-(((2-(3-Amino-6-methoxypyridin-2-yl)ethyl)(tert-butoxycarbonyl)amino)-methyl)-3-fluorophenyl)amino)-5-fluoro-4-(trifluoromethyl)benzoic acid 80d

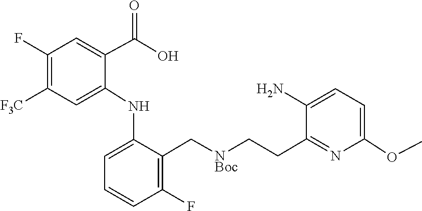 [SMILES: COc1ccc(N)c(CCN(Cc2c(F)cccc2Nc2cc(C(F)(F)F)c(F)cc2C(=O)O)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-3-fluorophenyl)amino)-5-fluoro-4-(trifluoromethyl)benzoic acid 80d (1.94 g, 95% yield) as a grayish brown solid. HPLC/MS 1.05 min (B), [M+H]⁺ 597.3. ¹H NMR (CDCl₃, 400 MHz): δ 9.23-9.53 (m, 1H), 7.85 (br d, J=11.0 Hz, 1H), 7.72 (br d, J=2.0 Hz, 2H), 7.14-7.27 (m, 3H), 7.04 (d, J=8.0 Hz, 1H), 6.86 (t, J=8.5 Hz, 1H), 6.45 (d, J=8.5 Hz, 1H), 4.59 (s, 2H), 3.77 (s, 3H), 3.44 (br t, J=7.5 Hz, 2H), 2.94 (br s, 2H), 1.42 (s, 9H).

### Step E: tert-Butyl 2,9-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 80e

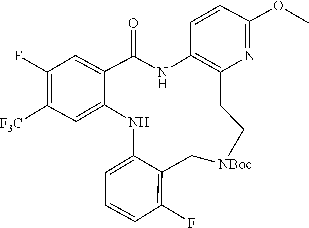 [SMILES: COc1ccc2c(n1)CCN(C(=O)OC(C)(C)C)Cc1c(F)cccc1Nc1cc(C(F)(F)F)c(F)cc1C(=O)N2]

Following the procedure outlined in Example 41, Step E, stirring the reaction mixture at RT for 4 h, tert-butyl 2,9-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 80e (1.18 g, 61% yield) was prepared as a tan solid. HPLC/MS 1.49 min (B), [M+H]⁺ 579.3. ¹H NMR (CDCl₃, 400 MHz): δ 11.06 (brs, 1H), 8.46 (d, J=11.2 Hz, 1H), 8.38 (d, J=8.8 Hz, 1H), 7.73 (d, J=5.9 Hz, 1H), 7.27 (br d, J=6.4 Hz, 1H), 6.87 (br t, J=8.8 Hz, 1H), 6.71-6.82 (m, 2H), 6.20 (br d, J=7.8 Hz, 1H), 4.43-4.79 (m, 1H), 4.01 (s, 3H), 3.32-3.71 (m, 1H), 2.32-2.58 (m, 2H), 2.16 (s, 2H), 1.19 (br s, 9H).

### Step F: tert-Butyl 2,9-difluoro-15-methoxy-19-oxo-3-(trifluoromethyl)-12,13-dihydro-19H-5,18-methanodibenzo-[c,f]pyrido[3,2-j][1,5,9]triazacyclotridecine-11(10H)-carboxylate 80f

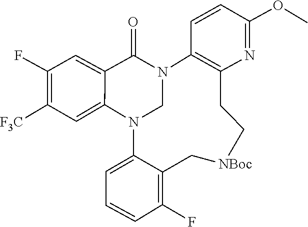 [SMILES: COc1ccc2c(n1)CCN(C(=O)OC(C)(C)C)Cc1c(F)cccc1N1CN2C(=O)c2cc(F)c(C(F)(F)F)cc21]

Following the procedure outlined in Example 41, Step F, stirring the reaction at 90° C. overnight, tert-butyl 2,9-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 80f (740 mg, 60% yield) was prepared as a light tan solid. HPLC/MS 1.42 min (B), [M+H]⁺ 591.1. ¹H NMR (CDCl₃, 400 MHz): δ 7.73 (br d, J=9.8 Hz, 1H), 7.22-7.31 (m, 2H), 7.00 (br t, J=8.6 Hz, 1H), 6.82 (br d, J=7.8 Hz, 1H), 6.46 (br d, J=8.8 Hz, 1H), 6.31 (br d, J=4.9 Hz, 1H), 5.39 (br d, J=11.2 Hz, 1H), 4.95 (br d, J=14.2 Hz, 1H), 4.40 (br d, J=11.2 Hz, 1H), 3.62-3.90 (m, 5H), 2.78-2.88 (m, 1H), 2.59-2.71 (m, 1H), 2.34 (br s, 1H), 1.31 (s, 9H).

### Step G: 2,9-Difluoro-3-(trifluoromethyl)-10,11,12,13-tetrahydro-19H-5,18-methano-dibenzo[c,f]pyrido[3,2-j][1,5,9]triazacyclotridecine-15,19(14H)-dione hydrochloride

### Example 80

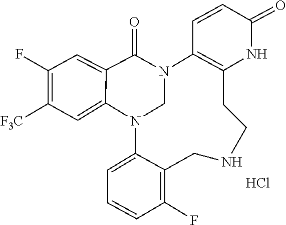 [SMILES: O=C1c2cc(F)c(C(F)(F)F)cc2N2CN1c1ccc(=O)[nH]c1CCNCc1c(F)cccc12 ; Cl]

Following the procedure outlined in Example 41, Step G, stirring the reaction mixture at 90° C. overnight, 2,9-difluoro-3-(trifluoromethyl)-10,11,12,13-tetrahydro-19H-5,18-methano-dibenzo[c,]pyrido[3,2-j][1,5,9]triazacyclotridecine-15,19(14H)-dione hydrochloride (505 mg, 77% yield) was prepared as a white solid. HPLC/MS 1.00 min (B), [M+H]⁺ 477.1. ¹H NMR (DMSO-d₆, 400 MHz): δ 10.50 (br s, 1H), 8.98-9.12 (m, 1H), 7.96 (d, J=10.5 Hz, 1H), 7.77 (td, J=8.3, 6.5 Hz, 1H), 7.47-7.59 (m, 3H), 6.47 (d, J=5.5 Hz, 1H), 6.41 (d, J=9.5 Hz, 1H), 5.51 (d, J=11.5 Hz, 1H), 5.14 (d, J=11.5 Hz, 1H), 4.21 (br s, 2H), 3.26-3.35 (m, 1H), 3.04-3.24 (m, 2H), 2.69-2.85 (m, 1H).

### Example 81

### 9-Chloro-2-fluoro-3-(trifluoromethyl)-10,11,12,13-tetrahydro-19H-5,18-methanodibenzo-[c,f]pyrido[3,2-j][1,5,9]triazacyclotridecine-15,19(14H)-dione hydrochloride

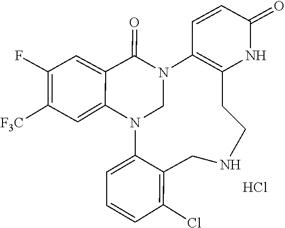 [SMILES: O=C1c2cc(F)c(C(F)(F)F)cc2N2CN1c1ccc(=O)[nH]c1CCNCc1c(Cl)cccc12 ; Cl]

### Step A: Methyl 2-((3-chloro-2-formylphenyl)amino)-5-fluoro-4-(trifluoromethyl)-benzoate 81a

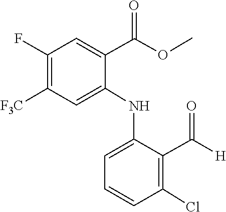 [SMILES: COC(=O)c1cc(F)c(C(F)(F)F)cc1Nc1cccc(Cl)c1C=O]

Following the preparation outlined in Example 7, Step A, substituting ethyl 2-bromo-5-(trifluoromethyl)benzoate with methyl 2-bromo-5-fluoro-4-(trifluoromethyl)benzoate, Int-3a with 2-amino-6-chlorobenzaldehyde, BINAP with Xantphos, toluene with 1,4-dioxane, and stirring the reaction at 100° C. overnight, methyl 2-((3-chloro-2-formylphenyl)amino)-5-fluoro-4-(trifluoromethyl)benzoate 81a (2.64 g, 710% yield) was prepared as a yellow solid. HPLC/MS 1.44 min (B), [M+H]⁺ 376.1. ¹H NMR (CDCl₃, 400 MHz): δ 11.57 (s, 1H), 10.60 (s, 1H), 7.76-7.85 (m, 2H), 7.29-7.35 (m, 1H), 7.20-7.26 (m, 1H), 6.96 (dd, J=7.8, 1.0 Hz, 1H), 3.98 (s, 3H).

### Step B: Methyl 2-((2-(((tert-Butoxycarbonyl)(2-(6-methoxy-3-nitropyridin-2-yl)ethyl-amino)methyl)-3-chlorophenyl)amino)-5-fluoro-4-(trifluoromethyl)benzoate 81b

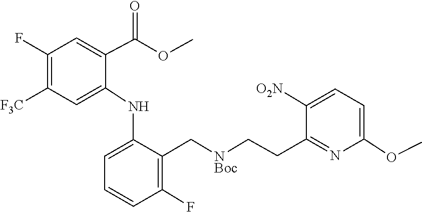 [SMILES: COC(=O)c1cc(F)c(C(F)(F)F)cc1Nc1cccc(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((3-chloro-2-formylphenyl)amino)-5-fluoro-4-(trifluoromethyl)benzoate 81a, methyl 2-((2-(((tert-butoxy-carbonyl)(2-(6-methoxy-3-nitropyridin-2-yl)ethyl)amino)methyl)-3-chlorophenyl)amino)-5-fluoro-4-(trifluoromethyl)benzoate 81b (3.08 g, 64% yield) was prepared as a clear oil. HPLC/MS 1.72 min (B), [M+H]⁺ 657.2. ¹H NMR (CDCl₃, 400 MHz): δ 9.21 (br s, 1H), 8.21 (d, J=9.0 Hz, 1H), 7.76 (d, J=11.0 Hz, 1H), 7.19-7.29 (m, 3H), 7.10 (d, J=5.5 Hz, 1H), 6.66 (br d, J=8.5 Hz, 1H), 4.82 (s, 2H), 3.97 (s, 3H), 3.94 (s, 3H), 3.64 (t, J=7.0 Hz, 2H), 3.32 (t, J=7.0 Hz, 2H), 1.31 (s, 9H).

### Step C: 2-((2-(((tert-Butoxycarbonyl)(2-(6-methoxy-3-nitropyridin-2-yl)ethyl)amino)-methyl)-3-chlorophenyl)amino)-5-fluoro-4-(trifluoromethyl)benzoic 81c

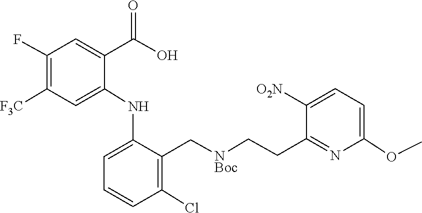 [SMILES: COc1ccc([N+](=O)[O-])c(CCN(Cc2c(Cl)cccc2Nc2cc(C(F)(F)F)c(F)cc2C(=O)O)C(=O)OC(C)(C)C)n1]

Following the procedure outlined in Example 41, Step C, stirring the reaction mixture at room temperature for 6 h, 2-((2-(((tert-butoxycarbonyl)(2-(6-methoxy-3-nitropyridin-2-yl)ethyl)-amino)methyl)-3-chlorophenyl)amino)-5-fluoro-4-(trifluoromethyl)benzoic 81c (2.79 g, 84% yield) was prepared as an orange powder. HPLC/MS 1.17 min (B), [M+H]⁺ 643.2. ¹H NMR (CDCl₃, 400 MHz): δ 9.13 (s, 1H), 8.12 (d, J=9.0 Hz, 1H), 7.71 (d, J=10.5 Hz, 1H), 7.11-7.21 (m, 3H), 6.84-7.01 (m, 1H), 6.57 (br d, J=9.0 Hz, 1H), 4.74 (s, 2H), 3.88 (s, 3H), 3.55 (t, J=7.0 Hz, 2H), 3.24 (t, J=7.0 Hz, 2H), 1.11-1.19 (m, 9H).

### Step D: 2-((2-(((2-(3-Amino-6-methoxypyridin-2-yl)ethyl)(tert-butoxycarbonyl)amino)-methyl)-3-chlorophenyl)amino)-5-fluoro-4-(trifluoromethyl)benzoic acid 81d

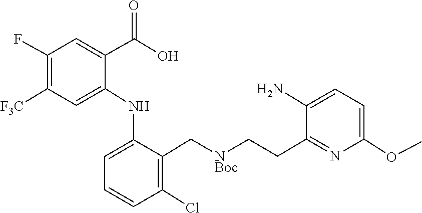 [SMILES: COc1ccc(N)c(CCN(Cc2c(Cl)cccc2Nc2cc(C(F)(F)F)c(F)cc2C(=O)O)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3-chlorophenyl)amino)-5-fluoro-4-(trifluoromethyl)benzoic acid 81d (2.27 g, 84% yield) as a brown solid. HPLC/MS 1.09 min (B), [M+H]⁺ 613.3. ¹H NMR (CDCl₃, 400 MHz): δ 9.61-9.81 (m, 1H) 7.82 (br d, J=11.0 Hz, 1H), 7.11-7.27 (m, 4H), 6.94 (d, J=8.5 Hz, 1H), 6.38 (d, J=8.5 Hz, 1H), 4.78 (s, 2H), 3.75 (s, 3H), 3.37 (br t, J=7.3 Hz, 2H), 2.75 (br s, 2H), 1.21-1.43 (m, 9H).

### Step E: tert-Butyl 9-chloro-2-fluoro-15-methoxy-19-oxo-3-(trifluoromethyl)-5,10,12,13,18,19-hexahydro-11H-dibenzo-[c,f]pyrido[3,2-j][1,5,9]triazacyclotridecine-11-carboxylate 81e

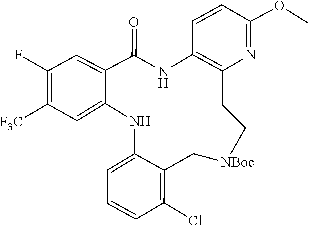 [SMILES: COc1ccc2c(n1)CCN(C(=O)OC(C)(C)C)Cc1c(Cl)cccc1Nc1cc(C(F)(F)F)c(F)cc1C(=O)N2]

Following the procedure outlined in Example 41, Step E, stirring the reaction mixture at room temperature for 2 h, tert-butyl 9-chloro-2-fluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 81e (1.11 g, 50% yield) was prepared as a tan solid. HPLC/MS 1.55 min (B), [M+H]⁺ 595.1. ¹H NMR (DMSO-d₆, 400 MHz): δ 10.16 (br s, 1H), 7.74-7.95 (m, 3H), 7.68 (br d, J=4.4 Hz, 1H), 6.98-7.11 (m, 1H), 6.92 (d, J=7.3 Hz, 1H), 6.68 (d, J=8.8 Hz, 1H), 6.29 (br d, J=7.8 Hz, 1H), 4.79 (s, 2H), 3.80 (s, 3H), 2.67-3.13 (m, 4H), 1.44 (br s, 9H).

### Step F: tert-Butyl 9-chloro-2-fluoro-15-methoxy-19-oxo-3-(trifluoromethyl)-12,13-dihydro-19H-5,18-methanodibenzo-[c,f]pyrido[3,2-j][1,5,9]triazacyclotridecine-11(10H)-carboxylate 81f

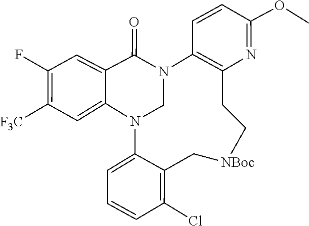 [SMILES: COc1ccc2c(n1)CCN(C(=O)OC(C)(C)C)Cc1c(Cl)cccc1N1CN2C(=O)c2cc(F)c(C(F)(F)F)cc21]

Following the procedure outlined in Example 41, Step F, stirring the reaction at 90° C. overnight, tert-butyl 9-chloro-2-fluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 81f (558 mg, 48% yield) was prepared as a light yellow solid. HPLC/MS 1.47 min (B), [M+H]⁺ 607.1. ¹H NMR (CDCl₃, 400 MHz): δ 7.81 (br d, J=9.5 Hz, 1H), 7.24-7.45 (m, 3H), 6.99 (br d, J=8.0 Hz, 1H), 6.53 (br d, J=8.5 Hz, 1H), 6.37 (br s, 1H), 5.47 (br d, J=11.0 Hz, 1H), 4.86-5.09 (m, 1H), 4.43 (br d, J=11.0 Hz, 1H), 3.96 (br s, 2H), 3.78 (s, 3H), 2.68-2.94 (m, 2H), 2.20-2.46 (m, 1H), 1.39 (br s, 9H).

### Step G: 9-Chloro-2-fluoro-3-(trifluoromethyl)-10,11,12,13-tetrahydro-19H-5,18-methanodibenzo[c,f]pyrido[3,2-i][1,5,9]triazacyclotridecine-15,19(14H)-dione hydrochloride Example 81

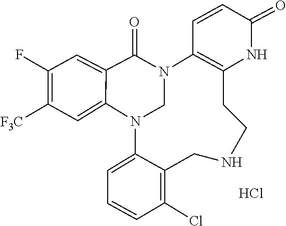 [SMILES: O=C1c2cc(F)c(C(F)(F)F)cc2N2CN1c1ccc(=O)[nH]c1CCNCc1c(Cl)cccc12 ; Cl]

Following the procedure outlined in Example 41, Step G, stirring the reaction mixture at 90° C. overnight, 9-chloro-2-fluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (364 mg, 75% yield) was prepared as a white solid. HPLC/MS 1.07 min (B), [M+H]⁺ 493.1. ¹H NMR (DMSO-d₆, 400 MHz): δ 10.51-10.71 (m, 1H), 8.88-9.05 (m, 1H), 7.96 (d, J=10.5 Hz, 1H), 7.69-7.83 (m, 2H), 7.64 (dd, J=8.0, 1.5 Hz, 1H), 7.61 (d, J=9.5 Hz, 1H), 6.53 (d, J=9.5 Hz, 1H), 6.46 (d, J=5.5 Hz, 1H), 5.50 (d, J=11.5 Hz, 1H), 5.23 (d, J=11.5 Hz, 1H), 4.39 (br d, J=14.0 Hz, 1H), 4.22 (br d, J=14.0 Hz, 1H), 3.32-3.45 (m, 1H), 3.10-3.29 (m, 2H), 2.77-2.92 (m, 1H).

### Example 82

### Step A: 2,8-Difluoro-15,19-dioxo-3-(trifluoromethyl)-10,11,12,13,14,15-hexahydro-19H-5,18-methanodibenzo[c,f]pyrido[3,2-j][1,5,9]triazacyclotridecine-9-carbonitrile hydrochloride

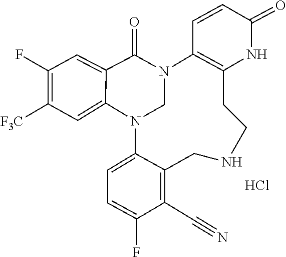 [SMILES: N#Cc1c(F)ccc2c1CNCCc1[nH]c(=O)ccc1N1CN2c2cc(C(F)(F)F)c(F)cc2C1=O ; Cl]

A vessel containing RuPhos-Pd-G3 (32.8 mg, 39.2 μmol), RuPhos (18.3 mg, 39.2 μmol), potassium ferrocyanide (41.3 mg, 97.9 μmol), and 9-chloro-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione Example 58 (100 mg, 196 μmol) was purged with nitrogen. Potassium acetate (4.80 mg, 48.9 μmol) and dioxane (2.18 mL) in degassed water (1.09 mL) were added via syringe, and the reaction mixture was heated to 100° C. for 3 h, cooled, transferred to a separatory funnel with EtOAc and washed with brine. The aqueous layer was extracted with EtOAc (2×), the combined organic extracts dried over Na₂SO₄, concentrated and purified by reverse-phase semi-prep HPLC (XSELECT CSH C18 column (150 mm×30 mm i.d. 5 μm packing diameter)), eluting with 20-99% AcCN in 10 mM Ammonium Bicarbonate in H₂O (adjusted to pH 10 with Ammonia) to afford partially pure material, which was further purified by reverse-phase semi-prep HPLC (XSELECT CSH C18 column (150 mm×30 mm i.d. 5 μm packing diameter)), eluting with 20-99% AcCN (0.1% formic acid) in water (0.1% formic acid). The pure product was dissolved in 4M HCl in dioxane and concentrated under reduced pressure to afford 2,8-difluoro-15,19-dioxo-3-(trifluoromethyl)-10,11,12,13,14,15-hexahydro-19H-5,18-methanodibenzo[c,f]pyrido[3,2-j]-[1,5,9]triazacyclotridecine-9-carbonitrile hydrochloride (53.0 mg, 48% yield) as a white powder. HPLC/MS 0.67 min (A), [M+H]⁺ 502.1. ¹H NMR (DMSO-d₆, 400 MHz): δ 2.92-3.14 (m, 2H), 3.93-4.29 (m, 1H), 4.35-4.67 (m, 1H), 5.02 (d, J=10.76 Hz, 1H), 5.43 (br s, 1H), 6.14-6.72 (m, 2H), 7.36-7.62 (m, 1H), 7.91 (br s, 2H), 8.05 (dd, J=8.80, 5.38 Hz, 1H), 8.97 (dt, J=7.70, 3.73 Hz, 1H), 9.90-10.21 (m, 1H), 11.76-12.19 (m, 1H).

### Example 83

### Step A: 2,3,8-Trifluoro-15,19-dioxo-10,11,12,13,14,15-hexahydro-19H-5,18-methano-dibenzo[c,f]pyrido[3,2-j][1,5,9]triazacyclotridecine-9-carbonitrile

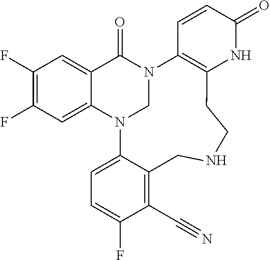 [SMILES: N#Cc1c(F)ccc2c1CNCCc1[nH]c(=O)ccc1N1CN2c2cc(F)c(F)cc2C1=O]

Following the procedure outlined in Example 82, using Example 64 as the substrate, 2,3,8-trifluoro-15,19-dioxo-10,11,12,13,14,15-hexahydro-19H-5,18-methanodibenzo[c,f]pyrido-[3,2-j][1,5,9]triazacyclotridecine-9-carbonitrile (6.6 mg, 14% yield) was prepared as a white solid. HPLC/MS 0.86 min (B), [M+H]⁺ 452.1. ¹H NMR (DMSO-d₆, 400 MHz): δ 11.84 (br s, 1H), 7.94 (dd, 1H, J=5.4, 8.8 Hz), 7.81 (dd, 1H, J=9.0, 10.5 Hz), 7.62 (t, 1H, J=8.8 Hz), 7.39 (d, 1H, J=9.8 Hz), 6.43 (dd, 1H, J=6.6, 12.5 Hz), 6.21 (br d, 1H, J=8.3 Hz), 5.35 (d, 1H, J=9.8 Hz), 4.98 (d, 1H, J=9.8 Hz), 3.99 (br t, 1H, J=11.5 Hz), 3.61 (br dd, 1H, J=3.7, 11.5 Hz), 2.96 (br d, 1H, J=12.7 Hz), 2.8-2.9 (m, 2H), 2.61 (br s, 1H), 2.24 (br d, 1H, J=12.2 Hz).

### Example 84

### Step A: 2,3,8-Trifluoro-9-hydroxy-10,11,12,13-tetrahydro-19H-5,18-methanodibenzo-[c,f]pyrido[3,2-j][1,5,9]triazacyclotridecine-15,19(14H)-dione, formic acid salt

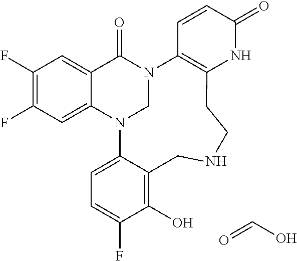 [SMILES: O=C1c2cc(F)c(F)cc2N2CN1c1ccc(=O)[nH]c1CCNCc1c2ccc(F)c1O ; O=CO]

A vessel containing 9-chloro-2,3,8-trifluoro-10,11,12,13-tetrahydro-19H-5,18-methanodibenzo[c,f]pyrido[3,2-j][1,5,9]triazacyclotridecine-15,19(14H)-dione hydrochloride Example 64 (200 mg, 402 μmol), NaO^{t}Bu (135 mg, 1.41 mmol), and adamtyl Brettphos G3 (81.4 mg, 80.4 μmol) was purged with nitrogen. Dioxane (3.22 mL) and degassed water (0.804 mL) were added via syringe, and the reaction mixture was heated to 80° C. for 2.5 h, cooled, transferred to a separatory funnel with EtOAc and washed with brine. The aqueous layer was with extracted with EtOAc and the combined organic extracts were dried over Na₂SO₄, filtered and concentrated under reduced pressure. The crude residue was purified by reverse-phase semi-prep HPLC (XSELECT CSH C18 column (150 mm×30 mm i.d. 5 μm packing diameter)) eluting with a 5-99% AcCN (0.1% formic acid) in water (0.1% formic acid) gradient. Product fractions were combined and evaporated under reduced pressure to afford 2,3,8-trifluoro-9-hydroxy-10,11,12,13-tetrahydro-19H-5,18-methanodibenzo[c,f]pyrido[3,2-j][1,5,9]triazacyclotridecine-15,19(14H)-dione, formic acid salt (81 mg, 40% yield) as a white solid. HPLC/MS 0.66 min (B), [M+H]⁺ 443.1. ¹H NMR (DMSO-d₆, 400 MHz): δ 2.17-2.32 (m, 1H), 2.76-3.09 (m, 6H), 3.68 (s, 4H), 4.97-5.08 (m, 1H), 5.26-5.34 (m, 1H), 6.15-6.27 (m, 1H), 6.36-6.46 (m, 1H), 6.91-6.99 (m, 1H), 7.17-7.29 (m, 1H), 7.34-7.44 (m, 1H), 7.73-7.85 (m, 1H), 8.15 (s, 1H), 11.77-11.93 (m, 1H).

### Example 85

### Step A: 2,8-Difluoro-9-hydroxy-3-(trifluoromethyl)-10,11,12,13-tetrahydro-19H-5,18-methanodibenzo[c,f]pyrido[3,2-i][1,5,9]triazacyclotridecine-15,19(14H)-dione hydrochloride

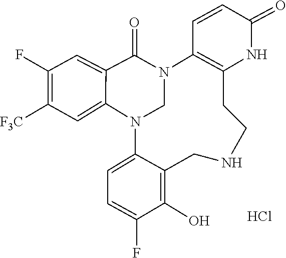 [SMILES: O=C1c2cc(F)c(C(F)(F)F)cc2N2CN1c1ccc(=O)[nH]c1CCNCc1c2ccc(F)c1O ; Cl]

Following the procedure outlined in Example 84, using Example 58 as the substrate, neutral product was synthesized, dissolved in methanol, to which was added hydrogen chloride in dioxane. This solution was concentrated to afford 2,8-difluoro-9-hydroxy-3-(trifluoromethyl)-10,11,12,13-tetrahydro-19H-5,18-methanodibenzo[c,f]pyrido[3,2-j][1,5,9]-triazacyclotridecine-15,19(14H)-dione hydrochloride (8 mg, 9% yield) as a white solid. HPLC/MS 0.66 min (A), [M+H]⁺ 493.1. ¹H NMR (DMSO-d₆, 400 MHz): δ 2.99-3.31 (m, 6H), 4.14 (br s, 2H), 4.88 (d, J=11.74 Hz, 1H), 5.43 (d, J=11.25 Hz, 1H), 6.26-6.39 (m, 1H), 6.47 (d, J=5.87 Hz, 1H), 7.08 (dd, J=8.80, 4.40 Hz, 1H), 7.35-7.66 (m, 2H), 7.91 (d, J=10.27 Hz, 1H), 8.17-8.53 (m, 1H), 9.24-9.56 (m, 1H), 11.10 (s, 2H), 11.94 (br s, 1H).

### Example 86

### 2,3-Difluoro-8-(trifluoromethoxy)-10,11,12,13-tetrahydro-19H-5,18-methanodibenzo[c,f]pyrido[3,2-j][1,5,9]triazacyclotridecine-15,19(14H)-dione hydrochloride

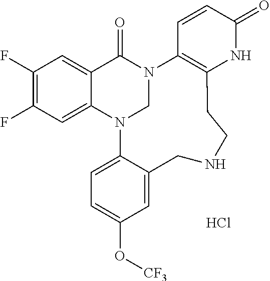 [SMILES: O=C1c2cc(F)c(F)cc2N2CN1c1ccc(=O)[nH]c1CCNCc1cc(OC(F)(F)F)ccc12 ; Cl]

### Step A: Methyl 4,5-difluoro-2-((2-formyl-4-(trifluoromethoxy)phenyl)amino)benzoate 86a

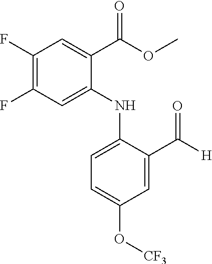 [SMILES: COC(=O)c1cc(F)c(F)cc1Nc1ccc(OC(F)(F)F)cc1C=O]

Following the preparation outlined in Example 7, Step A, substituting ethyl 2-bromo-5-(trifluoromethyl)benzoate with methyl 2-amino-4,5-difluorobenzoate, Int-3a with 2-bromo-5-(trifluoromethoxy)benzaldehyde, BINAP with Xantphos, toluene with 1,4-dioxane, and stirring the reaction mixture at 80° C. overnight, methyl 4,5-difluoro-2-((2-formyl-4-(trifluoromethoxy)phenyl)amino)benzoate 86a (375 mg, 57% yield) was prepared as a yellow solid. HPLC/MS 1.39 min (B), [M+H]⁺ 376.0. ¹H NMR (CDCl₃, 400 MHz): δ 11.26 (s, 1H), 9.97 (s, 1H), 7.87 (dd, J=11.0, 9.0 Hz, 1H), 7.55 (d, J=2.4 Hz, 1H), 7.49 (d, J=8.8 Hz, 1H), 7.28-7.42 (m, 2H), 3.96 (s, 3H).

### Step B: Methyl 2-((2-(((tert-butoxycarbonyl)(2-(6-methoxy-3-nitropyridin-2-yl)ethyl-amino)methyl)-4-(trifluoromethoxy)phenyl)amino)-4,5-difluorobenzoate 86b

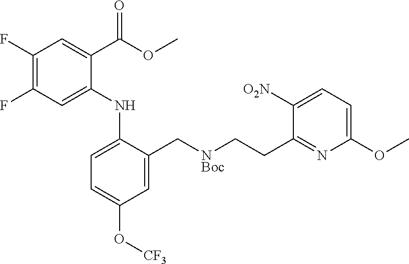 [SMILES: COC(=O)c1cc(F)c(F)cc1Nc1ccc(OC(F)(F)F)cc1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4,5-difluoro-2-((2-formyl-4-(trifluoromethoxy)phenyl)amino)benzoate 86a, methyl 2-((2-(((tert-butoxycarbonyl)(2-(6-methoxy-3-nitropyridin-2-yl)ethyl)amino)methyl)-4-(trifluoromethoxy)phenyl)amino)-4,5-difluorobenzoate 86b (4.66 g, 70% yield) was prepared as a clear oil. HPLC/MS 1.67 min (B), [M+H]⁺ 657.2. ¹H NMR (CDCl₃, 400 MHz): δ 9.18 (s, 1H) 8.19 (d, J=9.3 Hz, 1H) 7.76 (dd, J=11.2, 8.8 Hz, 1H) 7.25-7.27 (m, 1H) 7.12-7.22 (m, 2H) 6.64 (br d, J=8.8 Hz, 1H) 6.33-6.54 (m, 1H) 4.34-4.57 (m, 2H) 3.87-3.97 (m, 6H) 3.65 (br s, 2H) 3.28 (br s, 2H) 1.37 (s, 9H).

### Step C: 6-((2-(((tert-Butoxycarbonyl)(2-(6-methoxy-3-nitropyridin-2-yl)ethyl)amino)-methyl)-4-fluorophenyl)amino)-2-fluoro-3-(trifluoromethyl)benzoic acid 86c

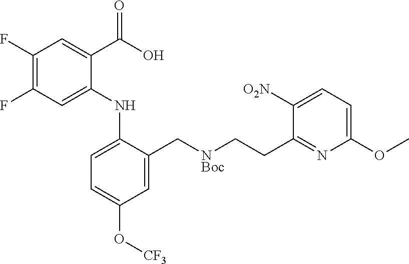 [SMILES: COc1ccc([N+](=O)[O-])c(CCN(Cc2cc(OC(F)(F)F)ccc2Nc2cc(F)c(F)cc2C(=O)O)C(=O)OC(C)(C)C)n1]

Following the procedure outlined in Example 41, Step C, stirring the reaction mixture at 23° C. for 2 h, 6-((2-(((tert-butoxycarbonyl)(2-(6-methoxy-3-nitropyridin-2-yl)ethyl)amino)-methyl)-4-fluorophenyl)amino)-2-fluoro-3-(trifluoromethyl)benzoic acid 86c (4.79 g, 100% yield) was prepared as a light yellow powder. HPLC/MS 1.15 min (B), [M+H]⁺ 643.2. ¹H NMR (CDCl₃, 400 MHz): δ 9.18 (br s, 1H), 8.19 (d, J=9.3 Hz, 1H), 7.69 (br s, 1H), 7.16-7.25 (m, 1 H), 7.10 (br s, 2H), 6.64 (br d, J=8.8 Hz, 1H), 6.36-6.55 (m, 1H), 4.56 (br s, 2H), 3.93 (s, 3H), 3.72 (br t, J=6.1 Hz, 2H), 3.31 (t, J=7.1 Hz, 2H), 1.40 (br s, 9H).

### Step D: 2-((2-(((2-(3-Amino-6-methoxypyridin-2-yl)ethyl)(tert-butoxycarbonyl)amino)-methyl)-4-(trifluoromethoxy)phenyl)amino)-4,5-difluorobenzoic acid 86d

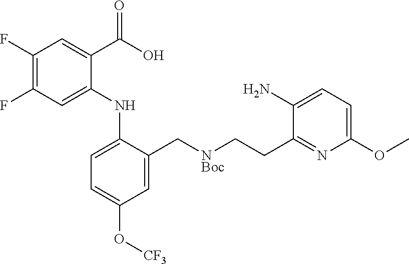 [SMILES: COc1ccc(N)c(CCN(Cc2cc(OC(F)(F)F)ccc2Nc2cc(F)c(F)cc2C(=O)O)C(=O)OC(C)(C)C)n1]

Following the procedure outlined in Example 44, Step D, 2-((2-(((2-(3-amino-6-methoxypyridin-2-yl)ethyl)(tert-butoxycarbonyl)amino)methyl)-4-(trifluoromethoxy)phenyl)-amino)-4,5-difluorobenzoic acid 86d (1.16 g, 100% yield) was prepared as a gray solid. HPLC/MS 1.05 min (B), [M+H]⁺ 613.2. ¹H NMR (CDCl₃, 400 MHz): δ 9.09-9.35 (m, 1H), 7.75 (br s, 1H), 7.20-7.25 (m, 1H), 6.98-7.19 (m, 3H), 6.37-6.58 (m, 2H), 4.43 (br s, 2H), 3.77 (s, 3H), 3.49-3.57 (m, 2H), 2.90 (br s, 2H), 1.44 (br s, 9H).

### Step E: tert-Butyl 2,3-difluoro-15-methoxy-19-oxo-8-(trifluoromethoxy)-5,10,12,13,18,19-hexahydro-11H-dibenzo[c,f]pyrido[3,2-j][1,5,9]triazacyclotridecine-11-carboxylate 86e

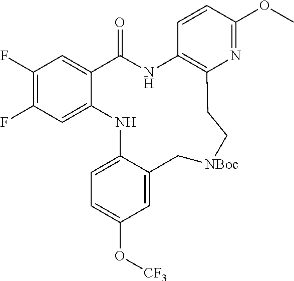 [SMILES: COc1ccc2c(n1)CCN(C(=O)OC(C)(C)C)Cc1cc(OC(F)(F)F)ccc1Nc1cc(F)c(F)cc1C(=O)N2]

Following the procedure outlined in Example 41, Step E, stirring the reaction mixture at RT overnight, tert-butyl 2,3-difluoro-15-methoxy-19-oxo-8-(trifluoromethoxy)-5,10,12,13,18,19-hexahydro-11H-dibenzo[c,f]pyrido[3,2-j][1,5,9]triazacyclotridecine-11-carboxylate 86e (827 mg, 18% yield) was prepared as a white solid. HPLC/MS 1.48 min (B), [M+H]⁺ 595.1. ¹H NMR (CDCl₃, 400 MHz): δ 10.93 (br s, 1H), 8.37 (dd, J=11.2, 9.3 Hz, 1H), 8.31 (d, J=8.8 Hz, 1H), 7.22 (t, J=8.4 Hz, 1H), 7.13 (dd, J=8.8, 1.5 Hz, 1H), 6.68 (d, J=8.8 Hz, 1H), 6.59 (br s, 1H), 6.39 (d, J=8.8 Hz, 1H), 3.61-4.05 (m, 5H), 2.30 (br s, 2H), 1.13 (br s, 9H).

### Step F: tert-Butyl 2,3-difluoro-15-methoxy-19-oxo-8-(trifluoromethoxy)-12,13-dihydro-19H-5,18-methanodibenzo[c,f]pyrido[3,2-j][1,5,9]triazacyclotridecine-11(10H)-carboxylate 86f

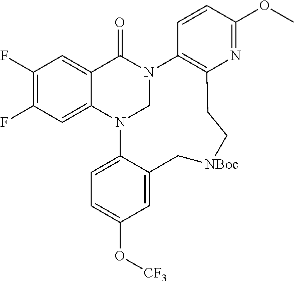 [SMILES: COc1ccc2c(n1)CCN(C(=O)OC(C)(C)C)Cc1cc(OC(F)(F)F)ccc1N1CN2C(=O)c2cc(F)c(F)cc21]

Following the procedure outlined in Example 41, Step F, stirring the reaction at 90° C. overnight, tert-butyl 2,3-difluoro-15-methoxy-19-oxo-8-(trifluoromethoxy)-12,13-dihydro-19H-5,18-methanodibenzo[c,f]pyrido[3,2-j][1,5,9]triazacyclotridecine-11(10H)-carboxylate 86f (640 mg, 76% yield) was prepared as a white solid. HPLC/MS 1.50 min (B), [M+H]⁺ 607.2. ¹H NMR (CDCl₃, 400 MHz): δ 7.92 (dd, J=10.3, 8.8 Hz, 1H) 7.57 (s, 1H) 7.47 (d, J=8.3 Hz, 1H) 7.26-7.29 (m, 1H) 7.17-7.22 (m, 1H) 6.65 (d, J=8.3 Hz, 1H) 6.10 (dd, J=10.8, 6.4 Hz, 1H) 5.58 (d, J=11.2 Hz, 1H) 4.83 (br d, J=14.7 Hz, 1H) 4.56 (d, J=10.8 Hz, 1H) 4.03 (br d, J=14.7 Hz, 1H) 3.89 (s, 4H) 3.06 (br d, J=2.9 Hz, 1H) 2.79 (br d, J=6.8 Hz, 1H) 2.56 (br t, J=13.2 Hz, 1H) 1.52 (s, 9H).

### Step G: 2,3-Difluoro-8-(trifluoromethoxy)-10,11,12,13-tetrahydro-19H-5,18-methanodibenzo[c,f]pyrido[3,2-j][1,5,9]triazacyclotridecine-15,19(14H)-dione hydrochloride Example 86

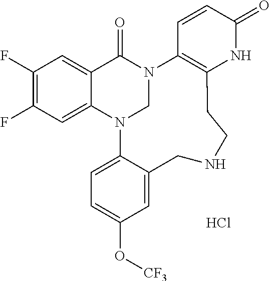 [SMILES: O=C1c2cc(F)c(F)cc2N2CN1c1ccc(=O)[nH]c1CCNCc1cc(OC(F)(F)F)ccc12 ; Cl]

Following the procedure outlined in Example 41, Step G, stirring the reaction mixture at 90° C. overnight, 2,3-difluoro-8-(trifluoromethoxy)-10,11,12,13-tetrahydro-19H-5,18-methano-dibenzo[c,]pyrido[3,2-j][1,5,9]triazacyclotridecine-15,19(14H)-dione hydrochloride (433 mg, 77% yield) was prepared as a white solid. HPLC/MS 1.07 min (B), [M+H]⁺ 493.1. ¹H NMR (DMSO-d₆, 400 MHz): δ 10.73-10.92 (m, 1H), 8.89-9.13 (m, 1H), 7.94 (d, J=2.0 Hz, 1H), 7.87 (dd, J=10.3, 8.8 Hz, 1H), 7.55-7.73 (m, 2H), 7.44 (d, J=9.8 Hz, 1H), 6.26-6.37 (m, 2H), 5.45 (d, J=11.2 Hz, 1H), 4.96 (d, J=11.7 Hz, 1H), 4.09 (br s, 2H), 3.12-3.25 (m, 1H), 3.05 (br d, J=6.8 Hz, 1H), 2.88-3.00 (m, 1H), 2.61-2.73 (m, 1H).

### Example 87

### 2,3,9-Trifluoro-8-(trifluoromethoxy)-10,11,12,13-tetrahydro-19H-5,18-methanodibenzo[c,f]pyrido[3,2-j][1,5,9]triazacyclotridecine-15,19(14H)-dione hydrochloride

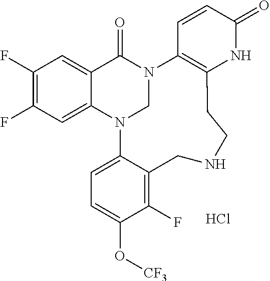 [SMILES: O=C1c2cc(F)c(F)cc2N2CN1c1ccc(=O)[nH]c1CCNCc1c2ccc(OC(F)(F)F)c1F ; Cl]

### Step A: Methyl 4,5-difluoro-2-((3-fluoro-2-formyl-4-(trifluoromethoxy)phenyl)-amino)-benzoate 87a

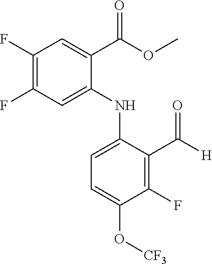 [SMILES: COC(=O)c1cc(F)c(F)cc1Nc1ccc(OC(F)(F)F)c(F)c1C=O]

Following the preparation outlined in Example 7, Step A, substituting ethyl 2-bromo-5-(trifluoromethyl)benzoate with methyl 2-amino-4,5-difluorobenzoate, Int-3a with 6-bromo-2-fluoro-3-(trifluoromethoxy)benzaldehyde, BINAP with Xantphos, toluene with 1,4-dioxane, and stirring the reaction mixture at 80° C. for 15 h, methyl 4,5-difluoro-2-((3-fluoro-2-formyl-4-(trifluoromethoxy)phenyl)amino)benzoate 87a (4.20 g, 61% yield) was prepared as a yellow solid. HPLC/MS 1.41 min (B), [M+H]⁺ 394.0. ¹H NMR (CDCl₃, 400 MHz): δ 11.50 (s, 1H), 10.41 (s, 1H), 7.87 (dd, J=10.8, 8.8 Hz, 1H), 7.37-7.41 (m, 1H), 7.33-7.37 (m, 1H), 7.15 (dd, J=9.5, 1.2 Hz, 1H), 3.95 (s, 3H).

### Step B: Methyl 2-((2-(((tert-butoxycarbonyl)(2-(6-methoxy-3-nitropyridin-2-yl)ethyl-amino)methyl)-3-fluoro-4-(trifluoromethoxy)phenyl)amino)-4,5-difluorobenzoate 87b

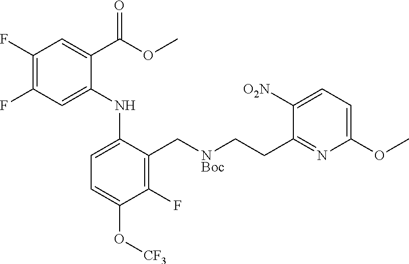 [SMILES: COC(=O)c1cc(F)c(F)cc1Nc1ccc(OC(F)(F)F)c(F)c1CN(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 4,5-difluoro-2-((3-fluoro-2-formyl-4-(trifluoromethoxy)phenyl)amino)benzoate 87a, methyl 2-((2-(((tert-butoxy-carbonyl)(2-(6-methoxy-3-nitropyridin-2-yl)ethyl)amino)methyl)-3-fluoro-4-(trifluoro-methoxy)phenyl)amino)-4,5-difluorobenzoate 87b (69 mg, 34% yield) was prepared as a yellow solid. HPLC/MS 1.67 min (B), [M+H]⁺ 675.2. ¹H NMR (CDCl₃, 400 MHz): δ 9.26 (br s, 1H) 8.21 (d, J=8.8 Hz, 1H) 7.75 (dd, J=11.0, 9.0 Hz, 1H) 7.20-7.25 (m, 1H) 7.10 (dd, J=9.0, 1.7 Hz, 1H) 6.66 (br d, J=9.3 Hz, 2H) 4.64 (s, 2H) 3.97 (s, 3H) 3.88 (s, 3H) 3.65 (t, J=7.1 Hz, 2H) 3.27 (t, J=7.1 Hz, 2H) 1.30 (s, 9H).

### Step C: 2-((2-(((tert-Butoxycarbonyl)(2-(6-methoxy-3-nitropyridin-2-yl)ethyl)amino)-methyl)-3-fluoro-4-(trifluoromethoxy)phenyl)amino)-4,5-difluorobenzoic acid 87c

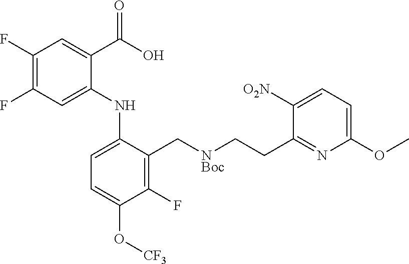 [SMILES: COc1ccc([N+](=O)[O-])c(CCN(Cc2c(Nc3cc(F)c(F)cc3C(=O)O)ccc(OC(F)(F)F)c2F)C(=O)OC(C)(C)C)n1]

Following the procedure outlined in Example 41, Step C, stirring the reaction mixture at ambient temperature for 2 h, 2-((2-(((tert-butoxycarbonyl)(2-(6-methoxy-3-nitropyridin-2-yl)ethyl)amino)methyl)-3-fluoro-4-(trifluoromethoxy)phenyl)amino)-4,5-difluorobenzoic acid 87c (5.09 g, 96% yield) was prepared as a yellow powder. HPLC/MS 1.18 min (B), [M+H]⁺ 661.1. ¹H NMR (CDCl₃, 400 MHz): δ 9.31 (br s, 1H), 8.22 (d, J=8.8 Hz, 1H), 7.79 (dd, J=10.8, 9.3 Hz, 1H), 7.20-7.26 (m, 1H), 7.10 (d, J=9.3 Hz, 1H), 6.61-6.71 (m, 2H), 4.60 (s, 2H), 3.98 (s, 3H), 3.71 (br t, J=6.8 Hz, 2H), 3.29 (t, J=6.8 Hz, 2H), 1.29 (s, 8H).

### Step D: 2-((2-(((2-(3-Amino-6-methoxypyridin-2-yl)ethyl)(tert-butoxycarbonyl)amino)-methyl)-3-fluoro-4-(trifluoromethoxy)phenyl)amino)-4,5-difluorobenzoic acid 87d

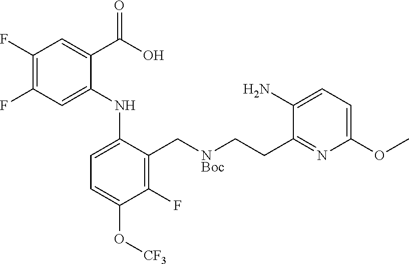 [SMILES: COc1ccc(N)c(CCN(Cc2c(Nc3cc(F)c(F)cc3C(=O)O)ccc(OC(F)(F)F)c2F)C(=O)OC(C)(C)C)n1]

Following the procedure outlined in Example 41, Step D, 2-((2-(((2-(3-amino-6-methoxypyridin-2-yl)ethyl)(tert-butoxycarbonyl)amino)methyl)-3-fluoro-4-(trifluoromethoxy)-phenyl)amino)-4,5-difluorobenzoic acid 87d (4.66 g, 96% yield) was prepared as a gray solid. HPLC/MS 1.07 min (B), [M+H]⁺ 631.1. ¹H NMR (CDCl₃, 400 MHz): δ 9.30-9.45 (m, 1H), 7.83 (dd, J=11.0, 9.0 Hz, 1H), 7.11-7.25 (m, 2H), 7.05 (dd, J=8.8, 1.5 Hz, 1H), 6.71 (dd, J=12.7, 6.8 Hz, 1H), 6.43 (d, J=8.8 Hz, 1H), 4.56 (s, 2H), 3.76 (s, 3H), 3.43 (br t, J=7.3 Hz, 2H), 2.92 (br d, J=6.8 Hz, 2H), 1.39 (s, 9H).

### Step E: tert-Butyl 2,3,9-trifluoro-15-methoxy-19-oxo-8-(trifluoromethoxy)-5,10,12,13,18,19-hexahydro-11H-dibenzo[c,f]pyrido[3,2-j][1,5,9]triazacyclotridecine-11-carboxylate 87e

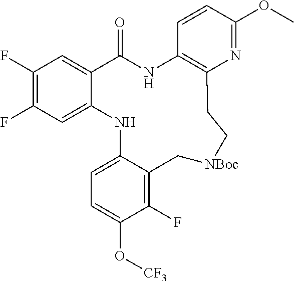 [SMILES: COc1ccc2c(n1)CCN(C(=O)OC(C)(C)C)Cc1c(ccc(OC(F)(F)F)c1F)Nc1cc(F)c(F)cc1C(=O)N2]

Following the procedure outlined in Example 41, Step E, stirring the reaction mixture at room temperature for 4 h, tert-butyl 2,3,9-trifluoro-15-methoxy-19-oxo-8-(trifluoromethoxy)-5,10,12,13,18,19-hexahydro-11H-dibenzo[c,f]pyrido[3,2-j][1,5,9]triazacyclotridecine-11-carboxylate 87e (2.67 g, 59% yield) was prepared as a white solid. HPLC/MS 1.52 min (B), [M+H]⁺ 613.1. ¹H NMR (CDCl₃, 400 MHz): δ 10.33-10.62 (m, 1H) 8.20-8.34 (m, 2H) 7.06-7.17 (m, 2H) 6.56-6.71 (m, 2H) 6.08 (d, J=8.8 Hz, 1H) 4.42-5.34 (m, 2H) 2.85-4.15 (m, 5H) 1.92-2.64 (m, 2H) 1.03 (s, 9H).

### Step F: tert-Butyl 2,3,9-trifluoro-15-methoxy-19-oxo-8-(trifluoromethoxy)-12,13-dihydro-19H-5,18-methanodibenzo[c,f]pyrido[3,2-j][1,5,9]triazacyclotridecine-11(10H)-carboxylate 87f

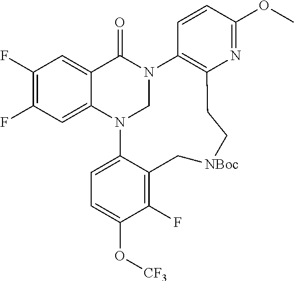 [SMILES: COc1ccc2c(n1)CCN(C(=O)OC(C)(C)C)Cc1c(ccc(OC(F)(F)F)c1F)N1CN2C(=O)c2cc(F)c(F)cc21]

Following the procedure outlined in Example 41, Step F, stirring the reaction at 90° C. for 39.5 h, tert-butyl 2,3,9-trifluoro-15-methoxy-19-oxo-8-(trifluoromethoxy)-12,13-dihydro-19H-5,18-methanodibenzo[c,f]pyrido[3,2-j][1,5,9]triazacyclotridecine-11(10H)-carboxylate 87f (1.58 g, 57% yield) was prepared as a light tan solid. HPLC/MS 1.47 min (B), [M+H]⁺ 625.2. ¹H NMR (CDCl₃, 400 MHz): δ 7.93 (dd, J=10.3, 8.8 Hz, 1H) 7.45 (d, J=8.3 Hz, 1H) 7.40 (t, J=8.2 Hz, 1H) 7.02 (dd, J=8.8, 2.0 Hz, 1H) 6.65 (d, J=8.8 Hz, 1H) 6.11 (dd, J=11.0, 6.1 Hz, 1H) 5.58 (d, J=11.2 Hz, 1H) 5.15 (br d, J=14.2 Hz, 1H) 4.54 (d, J=11.2 Hz, 1H) 4.00 (br d, J=14.7 Hz, 2H) 3.90 (s, 3H) 2.96-3.07 (m, 1H) 2.84 (br d, J=6.8 Hz, 1H) 2.41-2.58 (m, 1H) 1.48 (s, 9H).

### Step G: 2,3,9-Trifluoro-8-(trifluoromethoxy)-10,11,12,13-tetrahydro-19H-5,18-methanodibenzo[c,f]pyrido[3,2-j][1,5,9]triazacyclotridecine-15,19(14H)-dione hydrochloride

### Example 87

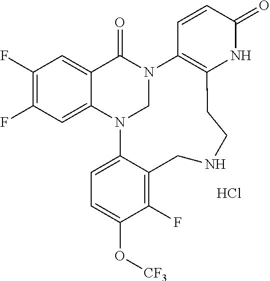 [SMILES: O=C1c2cc(F)c(F)cc2N2CN1c1ccc(=O)[nH]c1CCNCc1c2ccc(OC(F)(F)F)c1F ; Cl]

Following the procedure outlined in Example 41, Step G, stirring the reaction mixture at 90° C. overnight, 2,3,9-trifluoro-8-(trifluoromethoxy)-10,11,12,13-tetrahydro-19H-5,18-methanodibenzo[c,f]pyrido[3,2-j][1,5,9]triazacyclotridecine-15,19(14H)-dione hydrochloride (1.07 g, 77% yield) was prepared as a white solid. HPLC/MS 1.04 min (B), [M+H]⁺ 511.1. ¹H NMR (DMSO-d₆, 400 MHz): δ 10.63 (br s, 1H), 9.15-9.37 (m, 1H), 7.80-7.94 (m, 2H), 7.51 (dd, J=9.0, 1.2 Hz, 1H), 7.47 (d, J=9.8 Hz, 1H), 6.41 (dd, J=12.0, 6.6 Hz, 1H), 6.36 (d, J=9.3 Hz, 1H), 5.44 (d, J=11.2 Hz, 1H), 5.08 (d, J=11.2 Hz, 1H), 4.21 (br d, J=13.2 Hz, 1H), 3.96-4.13 (m, 1H), 3.27 (br d, J=6.4 Hz, 1H), 2.88-3.11 (m, 2H), 2.65-2.80 (m, 1H).

### Example 88

### Step A: 2-Fluoro-15,19-dioxo-8-(trifluoromethoxy)-10,11,12,13,14,15-hexahydro-19H-5,18-methanodibenzo[c,f]pyrido[3,2-j][1,5,9]triazacyclotridecine-3-carbonitrile hydrochloride

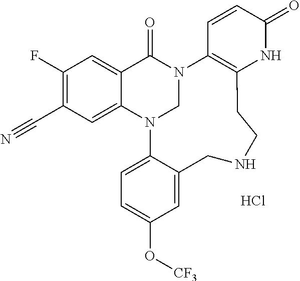 [SMILES: N#Cc1cc2c(cc1F)C(=O)N1CN2c2ccc(OC(F)(F)F)cc2CNCCc2[nH]c(=O)ccc21 ; Cl]

Following the preparation outlined in Example 55, Step A, substituting Example 86 as the substrate, stirring the reaction at 80° C. overnight, then acidifying the product in 4N HCl in dioxane, 2-fluoro-15,19-dioxo-8-(trifluoromethoxy)-10,11,12,13,14,15-hexahydro-19H-5,18-methano-dibenzo[c,f]pyrido[3,2-j][1,5,9]triazacyclotridecine-3-carbonitrile hydrochloride (21 mg, 10% yield) was prepared as a yellow solid. HPLC/MS 0.98 min (B), [M+H]⁺ 500.1. ¹H NMR (CD₃OD, 400 MHz): δ 7.86 (d, J=9.3 Hz, 1H) 7.60 (d, J=8.3 Hz, 1H) 7.48-7.54 (m, 2H) 7.36-7.46 (m, 1H) 6.80 (d, J=4.9 Hz, 1H) 6.43 (d, J=9.8 Hz, 1H) 5.39 (d, J=9.8 Hz, 1H) 5.29 (d, J=9.8 Hz, 1H) 4.02 (d, J=10.8 Hz, 1H) 3.49 (d, J=10.8 Hz, 1H) 3.09 (dt, J=8.4, 2.1 Hz, 2H) 2.95-3.04 (m, 1H) 2.36 (br d, J=11.2 Hz, 1H).

### Example 89

### Step A: 2,9-Difluoro-15,19-dioxo-8-(trifluoromethoxy)-10,11,12,13,14,15-hexahydro-19H-5,18-methanodibenzo[c,f]pyrido[3,2-j][1,5,9]triazacyclotridecine-3-carbonitrile hydrochloride

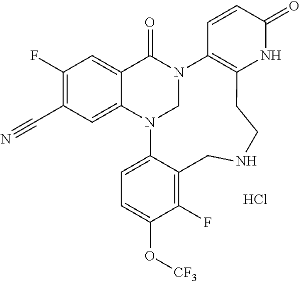 [SMILES: N#Cc1cc2c(cc1F)C(=O)N1CN2c2ccc(OC(F)(F)F)c(F)c2CNCCc2[nH]c(=O)ccc21 ; Cl]

Following the preparation outlined in Example 55, Step A, substituting Example 87 as the substrate, stirring the reaction at 80° C. overnight, then acidifying the product in 4N HCl in dioxane, 2,9-difluoro-15,19-dioxo-8-(trifluoromethoxy)-10,11,12,13,14,15-hexahydro-19H-5,18-methanodibenzo[c,f]pyrido[3,2-j][1,5,9]triazacyclotridecine-3-carbonitrile hydrochloride (65 mg, 31% yield) was prepared as a yellow solid. HPLC/MS 0.98 min (B), [M+H]⁺ 518.1. ¹H NMR (DMSO-d₆, 400 MHz): δ 7.84 (d, J=9.0 Hz, 1H), 7.63 (t, J=8.2 Hz, 1H), 7.48-7.52 (m, 1H), 7.22 (d, J=9.3 Hz, 1H), 7.01 (d, J=4.9 Hz, 1H), 6.07 (d, J=9.3 Hz, 1H), 5.34 (d, J=9.8 Hz, 1H), 5.11 (d, J=9.8 Hz, 1H), 3.60-3.71 (m, 2H), 2.75-2.91 (m, 2H), 2.38-2.46 (m, 1H), 2.19 (br s, 1H).

### Example 90

### 3,8-Difluoro-2-(trifluoromethyl)-10,11,12,13-tetrahydro-19H-5,18-methanodibenzo[f,i]-pyrido[2,3-b][1,4,8]triazacyclotridecine-15,19(14H)-dione

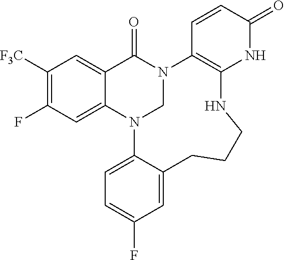 [SMILES: O=C1c2cc(C(F)(F)F)c(F)cc2N2CN1c1ccc(=O)[nH]c1NCCCc1cc(F)ccc12]

### Step A: Methyl 2-((2-(3-((tert-butoxycarbonyl)amino)prop-1-yn-1-yl)-4-fluorophenyl)-amino)-4-fluoro-5-(trifluoromethyl)benzoate 90a

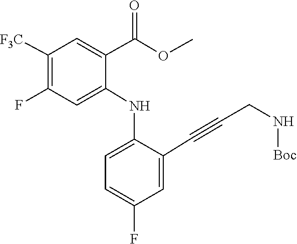 [SMILES: COC(=O)c1cc(C(F)(F)F)c(F)cc1Nc1ccc(F)cc1C#CCNC(=O)OC(C)(C)C]

Following the procedure outlined in Example 27, Step A, substituting Int-2 with Int-31, Int-3k with methyl 2-amino-4-fluoro-5-(trifluoromethyl)benzoate, and stirring the reaction mixture at 90° C. for 144 h, methyl 2-((2-(3-((tert-butoxycarbonyl)amino)prop-1-yn-1-yl)-4-fluorophenyl)amino)-4-fluoro-5-(trifluoromethyl)benzoate 90a (2.24 g, 49% yield) was prepared as a light yellow solid. HPLC/MS 1.50 min (A), [M+H-^{t}Bu]⁺ 429.1. ¹H NMR (DMSO-d₆, 400 MHz): δ 10.10 (s, 1H), 8.19 (d, 1H, J=8.3 Hz), 7.59 (dd, 1H, J=5.4, 8.8 Hz), 7.4-7.4 (m, 1H), 7.32 (s, 1H), 7.1-7.2 (m, 1H), 6.94 (d, 1H, J=13.7 Hz), 4.0-4.0 (m, 2H), 3.93 (s, 3H), 1.34 (s, 10H).

### Step B: Methyl 2-((2-(3-((tert-butoxycarbonyl)(6-methoxy-3-nitropyridin-2-yl)amino)-prop-1-yn-1-yl)-4-fluorophenyl)amino)-4-fluoro-5-(trifluoromethyl)benzoate 90b

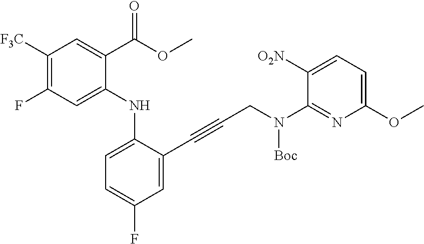 [SMILES: COC(=O)c1cc(C(F)(F)F)c(F)cc1Nc1ccc(F)cc1C#CCN(C(=O)OC(C)(C)C)c1nc(OC)ccc1[N+](=O)[O-]]

To methyl 2-((2-(3-((tert-butoxycarbonyl)amino)prop-1-yn-1-yl)-4-fluorophenyl)amino)-4-fluoro-5-(trifluoromethyl)benzoate 90a (2.09 g, 4.31 mmol) in DMF (40 mL) at 0° C. was added sodium hydride (242 mg, 6.04 mmol), the reaction mixture stirred for 20 min, to which was added 2-chloro-6-methoxy-3-nitropyridine (0.976 g, 5.18 mmol) in DMF (8 mL), dropwise. The reaction mixture was stirred at RT for 17 h, diluted with EtOAc, washed with H₂O (2×), brine, dried over Na₂SO₄, filtered and concentrated under reduced pressure. The crude residue was dissolved in DCM, absorbed onto a silica gel pre-packed column and purified by silica gel flash column chromatogarphy (220 g), eluting with a 50% EtOAc-heptanes to 100% EtOAc-heptanes gradient. Partially pure material was dissolved in DCM, absorbed onto a silica gel pre-packed column and purified by silica gel flash column chromatogarphy (220 g), eluting with 50% DCM-heptanes to 100% DCM-heptanes gradient. Product fractions were combined and evaporated under reduced pressure to afford methyl 2-((2-(3-((tert-butoxycarbonyl)(6-methoxy-3-nitropyridin-2-yl)-amino)prop-1-yn-1-yl)-4-fluorophenyl)amino)-4-fluoro-5-(trifluoromethyl)benzoate 90b (751 mg, 27% yield) as a white foam. HPLC/MS 1.61 min (A), [M+H-Boc]+537.1. ¹H NMR (DMSO-d₆, 400 MHz): δ 9.7-10.0 (m, 1H), 8.28 (d, 1H, J=8.8 Hz), 8.06 (d, 1H, J=8.3 Hz), 7.5-7.5 (m, 1H), 7.4-7.4 (m, 1H), 7.3-7.4 (m, 1H), 6.78 (d, 1H, J=8.8 Hz), 6.69 (d, 1H, J=13.7 Hz), 4.89 (s, 2H), 3.85 (s, 3H), 3.77 (s, 3H), 1.29 (br s, 11H).

### Step C: 2-((2-(3-((tert-Butoxycarbonyl)(6-methoxy-3-nitropyridin-2-yl)amino)prop-1-yn-1-yl)-4-fluorophenyl)amino)-4-fluoro-5-(trifluoromethyl)benzoic acid 90c

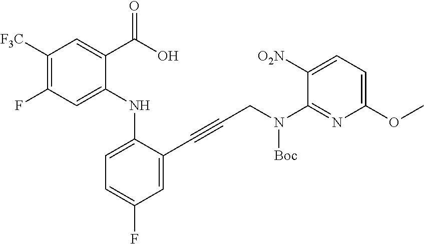 [SMILES: COc1ccc([N+](=O)[O-])c(N(CC#Cc2cc(F)ccc2Nc2cc(F)c(C(F)(F)F)cc2C(=O)O)C(=O)OC(C)(C)C)n1]

Following the procedure outlined in Example 27, Step B, stirring the reaction mixture at RT for 29 h, 2-((2-(3-tert-butoxycarbonyl)(6-methoxy-3-nitropyridin-2-yl)amino)prop-1-yn-1-yl)-4-fluorophenyl)amino)-5-(trifluoromethoxy)benzoic acid 90c (717 mg, 95% yield) was prepared as an off-white solid. HPLC/MS 1.51 min (A), [M+H-Boc]+523.1. ¹H NMR (DMSO-d₆, 400 MHz): δ 13.3-14.0 (m, 1H), 10.15 (br s, 1H), 8.28 (d, 1H, J=8.8 Hz), 8.09 (d, 1H, J=8.3 Hz), 7.52 (dd, 1H, J=5.4, 8.8 Hz), 7.3-7.4 (m, 2H), 6.78 (d, 1H, J=8.8 Hz), 6.68 (d, 1H, J=13.7 Hz), 4.88 (s, 2H), 3.88 (s, 3H), 1.30 (br s, 10H).

### Step D: 2-((2-(3-((3-Amino-6-methoxypyridin-2-yl)(tert-butoxycarbonyl)amino)propyl)-4-fluorophenyl)amino)-4-fluoro-5-(trifluoromethyl)benzoic acid 90d

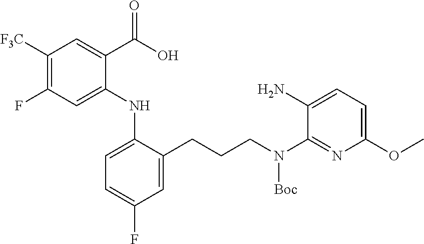 [SMILES: COc1ccc(N)c(N(CCCc2cc(F)ccc2Nc2cc(F)c(C(F)(F)F)cc2C(=O)O)C(=O)OC(C)(C)C)n1]

Following the procedure outlined in Example 27, Step C, stirring the reaction mixture at RT for 4 h, 2-((2-(3-((3-amino-6-methoxypyridin-2-yl)(tert-butoxycarbonyl)amino)propyl)-4-fluorophenyl)amino)-4-fluoro-5-(trifluoromethyl)benzoic acid 90d (649 mg, 74% yield) was prepared as a beige foam. HPLC/MS 1.33 min (A), [M+H]⁺ 597.2. ¹H NMR (DMSO-d₆, 400 MHz): δ 9.8-10.2 (m, 1H), 8.14 (d, 1H, J=8.8 Hz), 7.34 (s, 1H), 7.0-7.2 (m, 3H), 6.4-6.6 (m, 1H), 6.32 (d, 1H, J=13.7 Hz), 3.61 (s, 3H), 3.4-3.6 (m, 2H), 2.57 (br t, 2H, J=7.6 Hz), 1.70 (br s, 2H), 1.29 (br s, 9H).

### Step E: tertButyl 3,8-difluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 90e

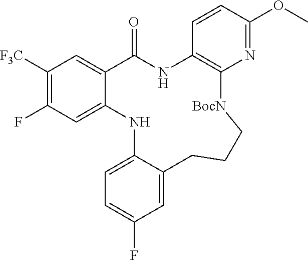 [SMILES: COc1ccc2c(n1)N(C(=O)OC(C)(C)C)CCCc1cc(F)ccc1Nc1cc(F)c(C(F)(F)F)cc1C(=O)N2]

Following the procedure outlined in Example 27, Step D, stirring the reaction mixture at RT for 20 h, tert butyl-3,8-difluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 90e (319.8 mg, 53% yield) was prepared as a light yellow foam. HPLC/MS 1.52 min (A), [M+H]⁺ 553.1. ¹H NMR (DMSO-d₆, 400 MHz): δ 8.5-9.0 (m, 2H), 7.8-8.4 (m, 1H), 7.33 (br d, 2H, J=2.9 Hz), 7.15 (br d, 1H, J=3.4 Hz), 6.89 (d, 1H, J=8.8 Hz), 6.36 (d, 1H, J=13.7 Hz), 3.9-4.0 (m, 1H), 3.83 (s, 3H), 2.5-2.7 (m, 1H), 1.5-1.8 (m, 2H), 1.16 (br s, 10H).

### Step F: tert-Butyl 3,8-difluoro-15-methoxy-19-oxo-2-(trifluoromethyl)-11,12-dihydro-19H-5,18-methanodibenzo[f,i]pyrido[2,3-b][1,4,8]triazacyclotridecine-13(10H)-carboxylate 90f

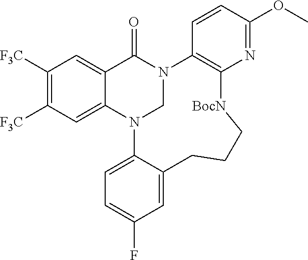 [SMILES: COc1ccc2c(n1)N(C(=O)OC(C)(C)C)CCCc1cc(F)ccc1N1CN2C(=O)c2cc(C(F)(F)F)c(C(F)(F)F)cc21]

Following the procedure outlined in Example 27, Step E, stirring the reaction mixture at 90° C. for 47 h, tert butyl-3,8-difluoro-15-methoxy-19-oxo-2-(trifluoromethyl)-11,12-dihydro-19H-5,18-methanodibenzo[f,i]pyrido[2,3-b][1,4,8]triazacyclotridecine-13(10H)-carboxylate 90f (258.4 mg, 77% yield) was prepared as an off white foam. HPLC/MS 1.49 min (A), [M+H]⁺ 591.1. ¹H NMR (DMSO-d₆, 400 MHz): δ 8.16 (d, 1H, J=8.3 Hz), 7.9-7.9 (m, 1H), 7.5-7.6 (m, 1H), 7.4-7.5 (m, 1H), 7.2-7.2 (m, 1H), 6.90 (d, 1H, J=8.8 Hz), 6.3-6.4 (m, 1H), 5.4-5.5 (m, 1H), 4.5-4.6 (m, 1H), 3.85 (s, 3H), 3.6-3.8 (m, 1H), 3.0-3.3 (m, 1H), 2.5-2.7 (m, 1H), 1.8-2.0 (m, 1H), 1.3-1.6 (m, 1H), 1.2-1.3 (m, 1H), 1.10 (s, 9H).

### Step G: 3,8-Difluoro-2-(trifluoromethyl)-10,11,12,13-tetrahydro-19H-5,18-methanodibenzo[f,i]pyrido[2,3-b][1,4,8]triazacyclotridecine-15,19(14H)-dione Example 90

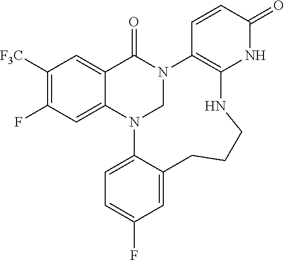 [SMILES: O=C1c2cc(C(F)(F)F)c(F)cc2N2CN1c1ccc(=O)[nH]c1NCCCc1cc(F)ccc12]

Following the procedure outlined in Example 27, Step F, stirring the reaction mixture at 90° C. for 72 h, 3,8-difluoro-2-(trifluoromethyl)-10,11,12,13-tetrahydro-19H-5,18-methanodibenzo-[f,i]pyrido[2,3-b][1,4,8]triazacyclotridecine-15,19(14H)-dione (130.5 mg, 62% yield) was prepared as a white solid. HPLC/MS 1.02 min (A), [M+H]⁺ 477.2. ¹H NMR (DMSO-d₆, 400 MHz): δ 10.4-11.3 (m, 1H), 8.13 (d, 1H, J=7.8 Hz), 7.48 (dd, 1H, J=5.4, 8.8 Hz), 7.3-7.4 (m, 2H), 7.22 (dt, 1H, J=2.9, 8.6 Hz), 6.20 (d, 1H, J=12.7 Hz), 5.76 (br dd, 2H, J=4.4, 8.8 Hz), 5.51 (d, 1H, J=11.7 Hz), 4.50 (d, 1H, J=11.2 Hz), 3.4-3.6 (m, 1H), 3.18 (d, 1H, J=4.9 Hz), 2.96 (br dd, 1H, J=4.4, 9.3 Hz), 2.79 (br d, 1H, J=10.3 Hz), 1.98 (br dd, 1H, J=4.9, 9.3 Hz), 1.35 (br d, 1H, J=5.9 Hz).

### Example 91

### 2,3,8-Trifluoro-10,11,12,13-tetrahydro-19H-5,18-methanodibenzo[f,i]pyrido[2,3-b]-[1,4,8]triazacyclotridecine-15,19(14H)-dione

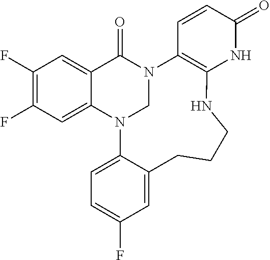 [SMILES: O=C1c2cc(F)c(F)cc2N2CN1c1ccc(=O)[nH]c1NCCCc1cc(F)ccc12]

### Step A: Methyl 2-((2-(3-((tert-butoxycarbonyl)(6-methoxy-3-nitropyridin-2-yl)amino)-prop-1-yn-1-yl)-4-fluorophenyl)amino)-4,5-difluorobenzoate 91a

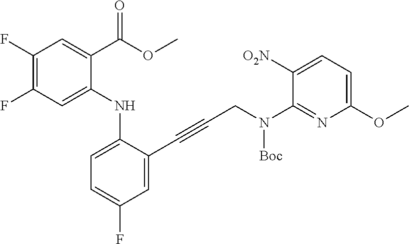 [SMILES: COC(=O)c1cc(F)c(F)cc1Nc1ccc(F)cc1C#CCN(C(=O)OC(C)(C)C)c1nc(OC)ccc1[N+](=O)[O-]]

Following the procedure outlined in Example 27, Step A, substituting methyl 2-bromo-5-(trifluoromethyl)benzoate with methyl 2-bromo-4,5-difluorobenzoate, stirring the reaction at 80° C. for 76 h, methyl 2-((2-(3-((tert-butoxycarbonyl)(6-methoxy-3-nitropyridin-2-yl)amino)prop-1-yn-1-yl)-4-fluorophenyl)amino)-4,5-difluorobenzoate 91a (3.90 g, 3100 yield) was prepared as a light yellow foam. HPLC/MS 1.55 min (A), [M+H]⁺ 587.1. ¹H NMR (DMSO-d₆, 400 MHz): δ 9.2-9.6 (m, 1H), 8.31 (d, 1H, J=8.8 Hz), 7.76 (dd, 1H, J=9.0, 11.5 Hz), 7.44 (br d, 1H, J=5.4 Hz), 7.33 (dd, 1H, J=2.9, 9.3 Hz), 7.2-7.3 (m, 1H), 6.92 (dd, 1H, J=6.8, 13.2 Hz), 6.80 (d, 1H, J=8.8 Hz), 4.91 (s, 2H), 3.87 (s, 3H), 3.73 (s, 3H), 1.31 (br s, 10H).

### Step B: 2-((2-(3-((tert-Butoxycarbonyl)(6-methoxy-3-nitropyridin-2-yl)amino)prop-1-yn-1-yl)-4-fluorophenyl)amino)-4,5-difluorobenzoic acid 91b

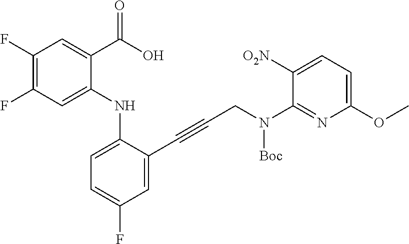 [SMILES: COc1ccc([N+](=O)[O-])c(N(CC#Cc2cc(F)ccc2Nc2cc(F)c(F)cc2C(=O)O)C(=O)OC(C)(C)C)n1]

Following the procedure outlined in Example 27, Step B, stirring the reaction mixture at RT for 25 h, then at 50° C. for 3 h, 2-((2-(3-((tert-butoxycarbonyl)(6-methoxy-3-nitropyridin-2-yl)amino)prop-1-yn-1-yl)-4-fluorophenyl)amino)-4,5-difluorobenzoic acid 91b (3.71 g, 91% a yield) was prepared as a beige solid. HPLC/MS 1.43 min (A), [M+H]573.1. ¹H NMR (DMSO-d₆, 400 MHz): δ 13.1-13.7 (m, 1H), 9.69 (br s, 1H), 8.31 (d, 1H, J=8.8 Hz), 7.77 (dd, 1H, J=9.0, 11.5 Hz), 7.45 (dd, 1H, J=5.4, 8.8 Hz), 7.2-7.3 (m, 2H), 6.91 (dd, 1H, J=7.1, 13.4 Hz), 6.80 (d, 1H, J=8.8 Hz), 4.91 (s, 2H), 3.89 (s, 3H), 1.32 (br s, 9H).

### Step C: 2-((2-(3-((3-Amino-6-methoxypyridin-2-yl)(tert-butoxycarbonyl)amino)propyl)-4-fluorophenyl)amino)-4,5-difluorobenzoic acid 91c

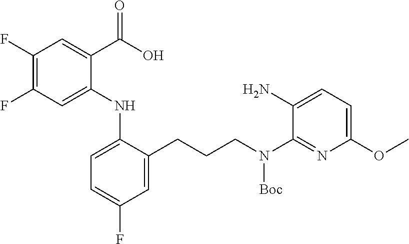 [SMILES: COc1ccc(N)c(N(CCCc2cc(F)ccc2Nc2cc(F)c(F)cc2C(=O)O)C(=O)OC(C)(C)C)n1]

Following the procedure outlined in Example 27, Step C, stirring the reaction mixture at room temperature for 72 h, 2-((2-(3-((3-amino-6-methoxypyridin-2-yl)(tert-butoxycarbonyl)-amino)propyl)-4-fluorophenyl)amino)-4,5-difluorobenzoic acid 91c (3.46 g, 79% yield) was prepared as a reddish-white foam. HPLC/MS 1.25 min (A), [M+H]⁺ 547.2.

### Step D: tert-Butyl 2,3,8-trifluoro-15-methoxy-19-oxo-5,10,11,12,18,19-hexahydro-13H-dibenzo[f,i]pyrido[2,3-b][1,4,8]triazacyclotridecine-13-carboxylate 91d

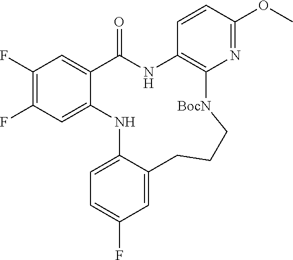 [SMILES: COc1ccc2c(n1)N(C(=O)OC(C)(C)C)CCCc1cc(F)ccc1Nc1cc(F)c(F)cc1C(=O)N2]

Following the procedure outlined in Example 27, Step D, substituting HATU for pyoxim, and stirring the reaction mixture at room temperature for 20 h, tert-butyl 2,3,8-trifluoro-15-methoxy-19-oxo-5,10,11,12,18,19-hexahydro-13H-dibenzo[f,i]pyrido[2,3-b][1,4,8]triazacyclo-tridecine-13-carboxylate 91d (2.56 g, 73% yield) was prepared as a white solid. HPLC/MS 1.41 min (A), [M+H]⁺ 529.2. ¹H NMR (DMSO-d₆, 400 MHz): δ 9.1-10.1 (m, 1H), 8.2-8.8 (m, 1H), 7.7-8.2 (m, 2H), 7.21 (br d, 1H, J=9.3 Hz), 7.03 (br s, 2H), 6.84 (d, 1H, J=8.8 Hz), 3.9-4.1 (m, 1H), 3.82 (s, 4H), 2.55 (br s, 2H), 1.6-2.0 (m, 1H), 1.0-1.5 (m, 12H).

### Step E: tert-Butyl 2,3,8-trifluoro-15-methoxy-19-oxo-11,12-dihydro-19H-5,18-methano-dibenzo[f,i]pyrido[2,3-b][1,4,8]triazacyclotridecine-13(10H)-carboxylate 91e

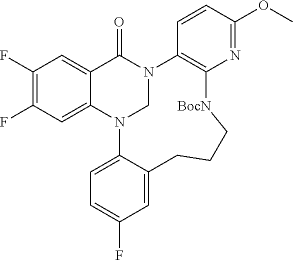 [SMILES: COc1ccc2c(n1)N(C(=O)OC(C)(C)C)CCCc1cc(F)ccc1N1CN2C(=O)c2cc(F)c(F)cc21]

Following the procedure outlined in Example 27, Step E, stirring the reaction mixture at 90° C. for 17 h, tert-butyl 2,3,8-trifluoro-15-methoxy-19-oxo-11,12-dihydro-19H-5,18-methanodibenzo[f,i]pyrido[2,3-b][1,4,8]triazacyclotridecine-13(10H)-carboxylate 91e (2.08 g, 78% yield) was prepared as a white solid. HPLC/MS 1.37 min (A), [M+H]⁺ 541.2. ¹H NMR (DMSO-d₆, 400 MHz): δ 7.8-7.9 (m, 2H), 7.4-7.5 (m, 1H), 7.3-7.4 (m, 1H), 7.16 (br d, 1H, J=2.9 Hz), 6.8-6.9 (m, 1H), 6.3-6.5 (m, 1H), 5.2-5.4 (m, 1H), 4.4-4.5 (m, 1H), 3.84 (s, 3H), 3.6-3.8 (m, 1H), 3.1-3.3 (m, 1H), 2.5-2.6 (m, 1H), 1.8-2.0 (m, 1H), 1.3-1.6 (m, 1H), 1.12 (s, 10H).

### Step F: 2,3,8-Trifluoro-10,11,12,13-tetrahydro-19H-5,18-methanodibenzo[f,i]pyrido[2,3-b][1,4,8]triazacyclotridecine-15,19(14H)-dione Example 91

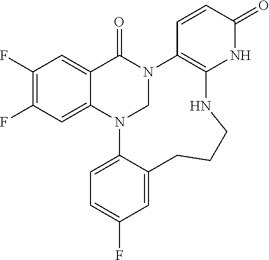 [SMILES: O=C1c2cc(F)c(F)cc2N2CN1c1ccc(=O)[nH]c1NCCCc1cc(F)ccc12]

Following the procedure outlined in Example 27, Step F, stirring the reaction mixture at 90° C. for 72 h, 2,3,8-trifluoro-10,11,12,13-tetrahydro-19H-5,18-methanodibenzo[f,i]pyrido[2,3-b][1,4,8]triazacyclotridecine-15,19(14H)-dione (204.3 mg, 47% yield) was prepared as a white solid. HPLC/MS 0.93 min (A), [M+H]⁺ 427.2. ¹H NMR (DMSO-d₆, 400 MHz): δ 10.5-11.1 (m, 1H), 7.83 (dd, 1H, J=9.0, 10.5 Hz), 7.43 (dd, 1H, J=5.4, 8.8 Hz), 7.3-7.4 (m, 2H), 7.20 (br d, 1H, J=2.9 Hz), 6.20 (dd, 1H, J=6.6, 12.0 Hz), 5.6-5.8 (m, 2H), 5.45 (d, 1H, J=11.2 Hz), 4.3-4.6 (m, 1H), 3.33 (s, 4H), 3.0-3.1 (m, 1H), 2.6-2.7 (m, 1H), 2.5-2.6 (m, 1H), 1.9-2.1 (m, 1H), 1.3-1.5 (m, 1H).

### Example 92

### 2,8,9-Trifluoro-3-(trifluoromethyl)-10,11,12,13-tetrahydro-19H-5,18-methanodibenzo[f,i]-pyrido[2,3-b][1,4,8]triazacyclotridecine-15,19(14H)-dione

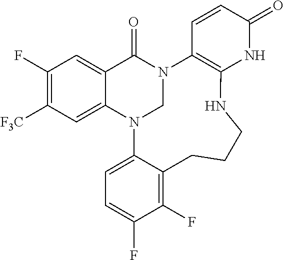 [SMILES: O=C1c2cc(F)c(C(F)(F)F)cc2N2CN1c1ccc(=O)[nH]c1NCCCc1c2ccc(F)c1F]

### Step A: tert-Butyl (3-(6-amino-2,3-difluorophenyl)prop-2-yn-1-yl)(6-methoxy-3-nitro-pyridin-2-yl)carbamate 92a

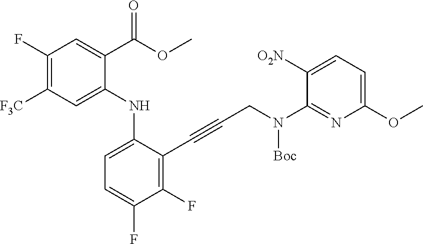 [SMILES: COC(=O)c1cc(F)c(C(F)(F)F)cc1Nc1ccc(F)c(F)c1C#CCN(C(=O)OC(C)(C)C)c1nc(OC)ccc1[N+](=O)[O-]]

To 7, 20 mL microwave vials was added Int-3m (1.10 g, 2.53 mmol), Int-2e (838.5 mg, 2.79 mmol), Cs₂CO₃ (1.16 g, 3.55 mmol) and 1,4-dioxane (14.0 mL) followed by Pd(OAc)₂ (34.1 mg, 152 μmol) and xantphos (175.8 mg, 303.9 mol), the reaction mixtures purged with N₂ for 5 min, sealed, then stirred at 80° C. for 14 h. The vials were cooled to ambient temperature, combined, filtered through thin celite pad, and washed with EtOAc. The filtrate was concentrated under reduced pressure, and the crude residue purified by silica gel flash column chromatography (330 g), eluting with a 100% heptanes to 22-30% EtOAc-heptane gradient. Pure product fractions were combined and concentrated under reduced pressure. The impure fractions were concentrated and re-purified by silica gel flash column chromatography (80 g), eluting with a 100% to 5-25% EtOAc-heptanes gradient. Purified material from both purifications were combined and the solvent evaporated under reduced pressure to afford methyl 2-((2-(3-((tert-butoxycarbonyl)(6-methoxy-3-nitropyridin-2-yl)amino)prop-1-yn-1-yl)-3,4-difluorophenyl)amino)-5-fluoro-4-(trifluoromethyl)benzoate 92a (8.92 g, 77% yield) as a yellow foam. HPLC/MS 1.65 min (A), [M+H]⁺ 655.0. ¹H NMR (DMSO-d₆, 400 MHz): δ 9.26 (br s, 1H), 8.31 (d, 1H, J=8.8 Hz), 7.81 (d, 1H, J=10.8 Hz), 7.4-7.5 (m, 1H), 7.31 (d, 1H, J=5.9 Hz), 7.26 (br dd, 1H, J=3.4, 8.3 Hz), 6.81 (d, 1H, J=8.8 Hz), 4.95 (s, 2H), 3.87 (s, 3H), 3.79 (s, 3H), 1.3-1.4 (m, 9H).

### Step B: Methyl 2-((2-(3-((3-amino-6-methoxypyridin-2-yl)(tert-butoxycarbonyl)amino)-propyl)-3,4-difluorophenyl)amino)-5-fluoro-4-(trifluoromethyl)benzoate 92b

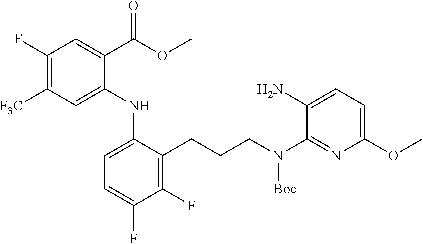 [SMILES: COC(=O)c1cc(F)c(C(F)(F)F)cc1Nc1ccc(F)c(F)c1CCCN(C(=O)OC(C)(C)C)c1nc(OC)ccc1N]

A solution of methyl 2-((2-(3-((tert-butoxycarbonyl)(6-methoxy-3-nitropyridin-2-yl)amino)prop-1-yn-1-yl)-3,4-difluorophenyl)amino)-5-fluoro-4-(trifluoromethyl)benzoate 92a (8.92 g, 13.6 mmol) in EtOH (120.0 mL) was evacuated and filled with nitrogen gas, then charged with 10% Pd—C(1.45 g, 1.36 mmol). The solution was evacuated and filled with hydrogen gas (1 atm), then stirred at ambient temperature for 22 h. The reaction mixture was filtered over celite and the filtrate was concentrated to provide methyl 2-((2-(3-((3-amino-6-methoxypyridin-2-yl)(tert-butoxycarbonyl)amino)propyl)-3,4-difluorophenyl)amino)-5-fluoro-4-(trifluoromethyl)-benzoate 92b (8.07 g, 89% yield) as a light brown foam. HPLC/MS 1.59 min (A), [M+H]⁺ 629.1 ¹H NMR (DMSO-d₆, 400 MHz): δ 9.03 (s, 1H), 7.84 (d, 1H, J=11.2 Hz), 7.3-7.4 (m, 1H), 7.18 (dd, 1H, J=3.9, 8.3 Hz), 7.1-7.2 (m, 1H, J=8.3 Hz), 6.88 (d, 1H, J=5.9 Hz), 6.4-6.5 (m, 1H, J=8.3 Hz), 4.2-4.4 (m, 2H), 3.88 (s, 3H), 3.59 (s, 3H), 3.4-3.6 (m, 2H), 2.6-2.7 (m, 2H), 1.71 (br s, 2H), 1.30 (s, 9H).

### Step C: 2-((2-(3-((3-Amino-6-methoxypyridin-2-yl)(tert-butoxycarbonyl)amino)propyl)-3,4-difluorophenyl)amino)-5-fluoro-4-(trifluoromethyl)benzoic acid hydrochloride 92c

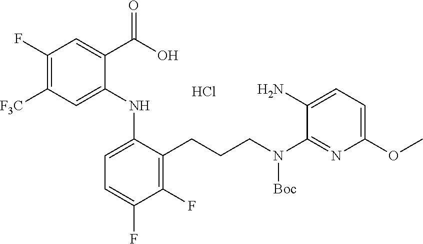 [SMILES: COc1ccc(N)c(N(CCCc2c(Nc3cc(C(F)(F)F)c(F)cc3C(=O)O)ccc(F)c2F)C(=O)OC(C)(C)C)n1 ; Cl]

To a solution of methyl 2-((2-(3-((3-amino-6-methoxypyridin-2-yl)(tert-butoxycarbonyl)amino)propyl)-3,4-difluorophenyl)amino)-5-fluoro-4-(trifluoromethyl)benzoate 92b (8.07 g, 12.1 mmol) in THF (90.0 mL) was added a solution of LiOH (2.89 g, 120.7 mmol) in H₂O (30.0 mL) and the reaction mixture was stirred at ambient temperature for 3 h. 6N HCl was added to the reaction mixture to adjust the pH-4. After dilution with EtOAc, the layers were separated, the organic extract washed with brine, dried over Na₂SO₄, filtered, and concentrated under reduced pressure to afford 2-((2-(3-((3-amino-6-methoxypyridin-2-yl)(tert-butoxycarbonyl)amino)propyl)-3,4-difluorophenyl)amino)-5-fluoro-4-(trifluoromethyl)benzoic acid, hydrochloride 92c (8.46 g, 100% yield) as a brown foam. HPLC/MS 1.06 min (A), [M+H]⁺ 615.1. ¹H NMR (DMSO-d₆, 400 MHz): δ 9.42 (br s, 1H), 7.84 (d, 1H, J=11.2 Hz), 7.35 (q, 1H, J=9.3 Hz), 7.21 (br dd, 1H, J=4.4, 8.8 Hz), 7.14 (br d, 1H, J=8.3 Hz), 6.89 (d, 1H, J=5.9 Hz), 6.50 (d, 1H, J=8.3 Hz), 3.60 (s, 3H), 3.3-3.6 (m, 2H), 2.65 (br t, 2H, J=7.3 Hz), 1.7-1.8 (m, 2H), 1.2-1.4 (m, 9H) (three exchangeable protons hidden, also HCl proton hidden).

### Step D: tert-Butyl 2,8,9-trifluoro-15-methoxy-19-oxo-3-(trifluoromethyl)-5,10,11,12,18,19-hexahydro-13H-dibenzo[f,i]pyrido[2,3-b][1,4,8]triazacyclotridecine-13-carboxylate 92d

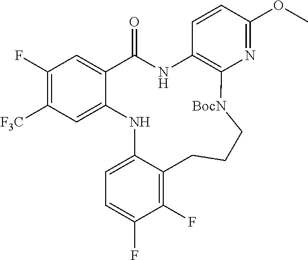 [SMILES: COc1ccc2c(n1)N(C(=O)OC(C)(C)C)CCCc1c(ccc(F)c1F)Nc1cc(C(F)(F)F)c(F)cc1C(=O)N2]

To a solution of 2-((2-(3-((3-amino-6-methoxypyridin-2-yl)(tert-butoxycarbonyl)-amino)propyl)-3,4-difluorophenyl)amino)-5-fluoro-4-(trifluoromethyl)benzoic acid, hydrochloride 92c (8.46 g, 12.9 mmol) in DMF (100.0 mL) was added DIEA (6.79 mL, 38.9 mmol) and pyoxim (12.3 g, 23.4 mmol). The reaction mixture was stirred at ambient temperature for 24 hour, diluted with EtOAc, the organics washed with H₂O (2×) and brine, dried over Na₂SO₄, filtered, and concentrated under reduced pressure. The crude residue was purified by silica gel flash column chromatography (2×330 g) eluting with a 100% heptanes to 30% EtOAc-heptanes gradient to afford tert-butyl 2,8,9-trifluoro-15-methoxy-19-oxo-3-(trifluoromethyl)-5,10,11,12,18,19-hexahydro-13H-dibenzo[f,i]pyrido[2,3-b][1,4,8]triazacyclotridecine-13-carboxylate 92d (5.65 g, 72% yield) as a yellow solid. HPLC/MS 1.52 min (A), [M+H]⁺ 597.1. ¹H NMR (CDCl₃, 400 MHz): δ 1.32 (s, 11H) 1.40-1.54 (m, 2H) 2.33 (br d, J=6.36 Hz, 1H) 2.70 (br s, 1H) 3.89-4.00 (m, 4H), 4.09 (br d, J=13.69 Hz, 1H) 4.22-4.42 (m, 1H) 6.75-6.79 (m, 1H) 6.82 (d, J=8.31 Hz, 1H) 7.11 (br d, J=8.31 Hz, 2H) 7.49 (br d, J=9.78 Hz, 2H) 7.83 (br d, J=4.40 Hz, 1H) 8.37 (br s, 1H).

### Step E: tert-Butyl 2,8,9-trifluoro-15-methoxy-19-oxo-3-(trifluoromethyl)-11,12-dihydro-19H-5,18-methanodibenzo[f,i]pyrido[2,3-b][1,4,8]triazacyclotridecine-13(10H)-carboxylate 92e

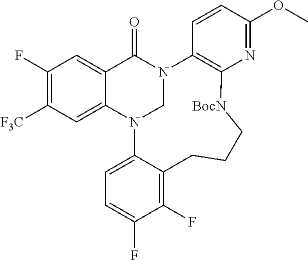 [SMILES: COc1ccc2c(n1)N(C(=O)OC(C)(C)C)CCCc1c(ccc(F)c1F)N1CN2C(=O)c2cc(F)c(C(F)(F)F)cc21]

To a solution of tert-butyl 2,8,9-trifluoro-15-methoxy-19-oxo-3-(trifluoromethyl)-5,10,11,12,18,19-hexahydro-13H-dibenzo[f,i]pyrido[2,3-b][1,4,8]triazacyclotridecine-13-carboxylate 92d (5.65 g, 9.47 mmol) in MeCN (95 mL) was added Cs₂CO₃ (18.5 g, 56.8 mmol) and CH₂I₂ (11.5 mL, 142.1 mmol). The reaction mixture was stirred at 85° C. for 32 h, cooled to ambient temperature, diluted with EtOAc and filtered. The organic layer was washed with brine (2×), dried over Na₂SO₄, filtered, and concentrated under reduced pressure. The crude residue was purified by silica gel flash column chromatography (330 g) eluting with a 100% heptanes to 40% EtOAc-heptanes gradient. Product fractions were combined and evaporated under reduced pressure to afford tert-butyl 2,8,9-trifluoro-15-methoxy-19-oxo-3-(trifluoromethyl)-11,12-dihydro-19H-5,18-methanodibenzo[f,i]pyrido[2,3-b][1,4,8]triazacyclotridecine-13(10H)-carboxylate 92e (4.86 g, 83% yield) as an off-white solid. HPLC/MS 1.48 min (A), [M+H]⁺ 609.1. ¹H NMR (DMSO-d₆, 400 MHz): δ 1.11 (s, 10H) 1.35-1.60 (m, 1H) 1.84-2.03 (m, 1H) 2.71-2.87 (m, 1H) 3.05-3.26 (m, 1H) 3.63-3.80 (m, 1H) 3.85 (s, 3H) 3.93-4.16 (m, 1H) 4.45-4.61 (m, 1H) 5.35 (br d, J=11.25 Hz, 1H) 6.71-6.83 (m, 1H) 6.91 (d, J=8.80 Hz, 1H) 7.31-7.56 (m, 2H) 7.81-8.01 (m, 2H).

### Step F: 2,8,9-Trifluoro-3-(trifluoromethyl)-10,11,12,13-tetrahydro-19H-5,18-methano-dibenzo[f,i]pyrido[2,3-b][1,4,8]triazacyclotridecine-15,19(14H)-dione 92f

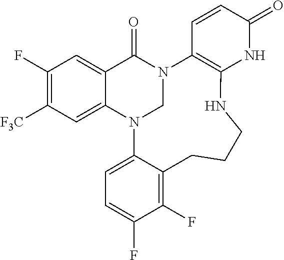 [SMILES: O=C1c2cc(F)c(C(F)(F)F)cc2N2CN1c1ccc(=O)[nH]c1NCCCc1c2ccc(F)c1F]

To tert-butyl 2,8,9-trifluoro-15-methoxy-19-oxo-3-(trifluoromethyl)-11,12-dihydro-19H-5,18-methanodibenzo[f,i]pyrido[2,3-b][1,4,8]triazacyclotridecine-13(10H)-carboxylate 92e (4.82 g, 7.92 mmol) was added LiCl (2.35 g, 55.4 mmol), and DMF (22.6 mL), followed by methanesulfonic acid (3.73 mL, 55.4 mmol), and the reaction mixture was heated to 92° C. for 4 h. The reaction mixture was cooled to ambient temperature, quenched with EtOAc and careful addition of of aq. sat'd NaHCO₃. The suspension was separated, the organic layer washed with H₂O (2×) and filtered to collect a yellow solid. The mother liquor was evaporated under reduced pressure, to which was added EtOAc (20 mL), the suspension sonicated, and the yellow solid filtered. The yellow solids were combined to afford 2,8,9-trifluoro-3-(trifluoromethyl)-10,11,12,13-tetrahydro-19H-5,18-methanodibenzo[f,i]pyrido[2,3-b][1,4,8]triazacyclotridecine-15,19(14H)-dione 92f (3.31 g, 84% yield). HPLC/MS 1.04 min (A), [M+H]⁺ 495.1. ¹H NMR (DMSO-d₆, 400 MHz): δ 1.32-1.46 (m, 1H) 1.92-2.12 (m, 1H) 2.65-2.80 (m, 1H) 2.81-2.93 (m, 1H) 2.94-3.07 (m, 1H) 3.45-3.63 (m, 1H) 4.45 (br d, J=10.76 Hz, 1H) 5.40 (d, J=11.25 Hz, 1H) 5.69-5.96 (m, 2H) 6.54 (d, J=5.87 Hz, 1H) 7.30-7.40 (m, 2H) 7.47 (d, J=10.27 Hz, 1H) 7.90 (d, J=10.27 Hz, 1H).

### Step G: 2,8,9-Trifluoro-3-(trifluoromethyl)-10,11,12,13-tetrahydro-19H-5,18-methano-dibenzo[f,i]pyrido[2,3-b][1,4,8]triazacyclotridecine-15,19(14H)-dione, methanesulfonic acid salt Example 92

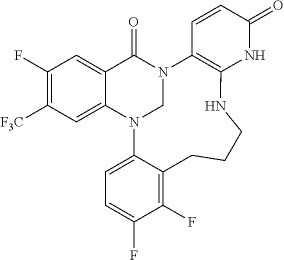 [SMILES: O=C1c2cc(F)c(C(F)(F)F)cc2N2CN1c1ccc(=O)[nH]c1NCCCc1c2ccc(F)c1F]

To a suspension of 2,8,9-trifluoro-3-(trifluoromethyl)-10,11,12,13-tetrahydro-19H-5,18-methanodibenzo[f,i]pyrido[2,3-b][1,4,8]triazacyclotridecine-15,19(14H)-dione 92f (3.31 g, 6.69 mmol) in MeCN (134 mL) was added methanesulfonic acid (478.0 μL, 7.36 mmol), the reaction mixture stirred at 40° C. for 1 h, then allowed to cool to ambient temperature. The suspension was filtered, rinsed with MeCN, and dried overnight to afford 2,8,9-trifluoro-3-(trifluoromethyl)-10,11,12,13-tetrahydro-19H-5,18-methanodibenzo[f,i]pyrido[2,3-b][1,4,8]triazacyclotridecine-15,19(14H)-dione, methanesulphonic acid salt 92 (3.50 g, 89% yield), as a white solid. HPLC/MS 1.04 min (A), [M+H]⁺ 495.1. ¹H NMR (DMSO-d₆, 400 MHz): δ 1.32-1.47 (m, 1H) 1.94-2.12 (m, 1H) 2.33 (d, J=3.42 Hz, 3H) 2.63-2.80 (m, 1H) 2.85 (br d, J=9.29 Hz, 1H) 2.94-3.12 (m, 1H) 3.47-3.62 (m, 1H) 4.47 (br d, J=11.25 Hz, 1H) 5.41 (d, J=11.25 Hz, 1H) 5.86 (br t, J=8.07 Hz, 1H) 5.90-6.20 (m, 1H) 6.54 (d, J=5.38 Hz, 1H) 7.31-7.55 (m, 3H) 7.90 (d, J=10.76 Hz, 1H).

### Example 93

### 3-Chloro-2,8-difluoro-10,11,12,13-tetrahydro-19H-5,18-methanodibenzo[f,i]pyrido[2,3-b]-[1,4,8]triazacyclotridecine-15,19(14H)-dione

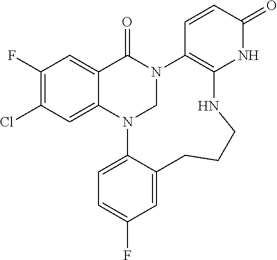 [SMILES: O=C1c2cc(F)c(Cl)cc2N2CN1c1ccc(=O)[nH]c1NCCCc1cc(F)ccc12]

### Step A: Methyl 2-((2-(3-((tert-butoxycarbonyl)amino)propyl)-4-fluorophenyl)amino)-4-chloro-5-fluorobenzoate 93a

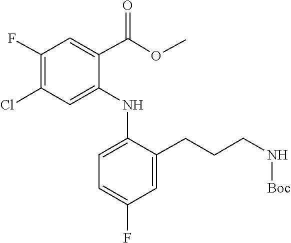 [SMILES: COC(=O)c1cc(F)c(Cl)cc1Nc1ccc(F)cc1CCCNC(=O)OC(C)(C)C]

Following the procedure outlined in Example 7, Step A, substituting methyl 2-bromo-5-(trifluoromethyl)benzoate with methyl 2-bromo-4-chloro-5-fluorobenzoate, Int-3a with Int-3g, toluene with 1,4-dioxane, and stirring the reaction mixture at 80° C. for 16 h, methyl 2-((2-(3-((tert-butoxycarbonyl)amino)propyl)-4-fluorophenyl)amino)-4-chloro-5-fluorobenzoate 93a (1.87 g, 79% yield) was prepared as a light brown solid. HPLC/MS 1.55 min (B), [M+H]⁺ 455.1. ¹H NMR (CDCl₃, 400 MHz): δ 9.07 (s, 1H), 7.75 (d, J=9.8 Hz, 1H), 7.23 (dd, J=8.3, 5.4 Hz, 1H), 7.04 (dd, J=9.3, 2.9 Hz, 1H), 6.94-7.01 (m, 1H), 6.69 (d, J=6.4 Hz, 1H), 4.64 (br s, 1H), 3.95 (s, 3H), 3.13-3.21 (m, 2H), 2.61 (dd, J=9.0, 7.1 Hz, 2H), 1.72-1.82 (m, 2H), 1.43 (s, 9H).

### Step B: Methyl 2-((2-(3-((tert-butoxycarbonyl)(6-methoxy-3-nitropyridin-2-yl)amino)-propyl)-4-fluorophenyl)amino)-4-chloro-5-fluorobenzoate 93b

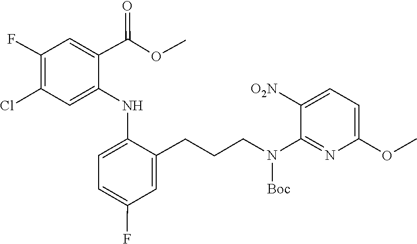 [SMILES: COC(=O)c1cc(F)c(Cl)cc1Nc1ccc(F)cc1CCCN(C(=O)OC(C)(C)C)c1nc(OC)ccc1[N+](=O)[O-]]

To methyl 2-((2-(3-((tert-butoxycarbonyl)amino)propyl)-4-fluorophenyl)amino)-4-chloro-5-fluorobenzoate 93a (0.626 g, 1.38 mmol) in DMF (15 mL) at 0° C. was added LHMDS (3.44 mL, 3.44 mmol). After 15 min, 2-chloro-6-methoxy-3-nitropyridine (0.337 g, 1.79 mmol) in DMF (5 mL) was added, and the reaction was stirred at RT overnight. The reaction mixture was cooled to 0° C., additional LHMDS (0.86 mL, 0.86 mmol) was added, and the reaction mixture was warmed to RT and stirred for 1.5 h. The reaction mixture was quenched with aq. sat'd NH₄Cl and extracted with EtOAc. The organic extract was washed with H₂O (3×), brine, dried over Na₂SO₄, filtered, concentrated under reduced pressure. The crude residue was purified by silica gel flash column chromatography (120 g), eluting with a 100% heptanes to 50% EtOAc-hepatanes gradient. Product fractions were combine and evaporated under reduced pressure to afford methyl 2-((2-(3-((tert-butoxycarbonyl)(6-methoxy-3-nitropyridin-2-yl)amino)propyl)-4-fluorophenyl)-amino)-4-chloro-5-fluorobenzoate 93b (467 mg, 53% yield) as a light brown solid. HPLC/MS 1.72 min (B), [M+H]⁺ 607.1. ¹H NMR (CD₃OD, 400 MHz): δ 8.94 (s, 1H), 8.19 (d, J=8.8 Hz, 1H), 7.67 (d, J=9.8 Hz, 1H), 7.19 (dd, J=8.8, 5.4 Hz, 1H), 7.13 (dd, J=9.3, 2.9 Hz, 1H), 7.02 (td, J=8.6, 2.9 Hz, 1H), 6.65 (d, J=8.8 Hz, 1H), 6.42 (d, J=6.4 Hz, 1H), 4.05 (br, s, 2H), 3.91 (s, 3H), 3.86 (s, 3H), 2.69 (br s, 2H), 1.95 (br s, 2H), 1.34 (br s, 9H).

### Step C: 2-((2-(3-((tert-butoxycarbonyl)(6-methoxy-3-nitropyridin-2-yl)amino)propyl)-4-fluorophenyl)amino)-4-chloro-5-fluorobenzoic acid 93c

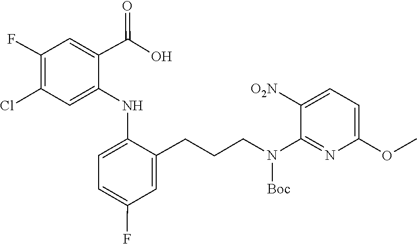 [SMILES: COc1ccc([N+](=O)[O-])c(N(CCCc2cc(F)ccc2Nc2cc(Cl)c(F)cc2C(=O)O)C(=O)OC(C)(C)C)n1]

Following the procedure outlined in Example 27, Step B, stirring the reaction mixture at RT for 22 h, 2-((2-(3-((tert-butoxycarbonyl)(6-methoxy-3-nitropyridin-2-yl)amino)propyl)-4-fluorophenyl)amino)-4-chloro-5-fluorobenzoic acid 93c (466 mg, 100% yield) was prepared as a light yellow glass. HPLC/MS 1.13 min (B), [M+H]⁺ 593.0. ¹H NMR (CD₃OD, 400 MHz): δ 8.21 (d, J=8.8 Hz, 1H), 7.69 (d, J=9.8 Hz, 1H), 7.21 (dd, J=8.3, 5.4 Hz, 1H), 7.13 (dd, J=9.3, 2.9 Hz, 1H), 7.02 (td, J=8.6, 2.9 Hz, 1H), 6.67 (d, J=8.8 Hz, 1H), 6.44 (d, J=6.4 Hz, 1H), 4.05 (s, 2H), 3.86 (s, 3H), 2.69 (br s, 2H), 1.96 (br dd, J=5.6, 3.2 Hz, 2H), 1.35 (br s, 9H).

### Step D: 2-((2-(3-((3-amino-6-methoxypyridin-2-yl)(tert-butoxycarbonyl)amino)propyl)-4-fluorophenyl)amino)-4-chloro-5-fluorobenzoic acid 93d

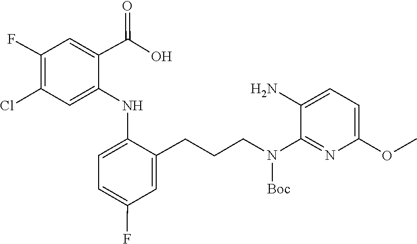 [SMILES: COc1ccc(N)c(N(CCCc2cc(F)ccc2Nc2cc(Cl)c(F)cc2C(=O)O)C(=O)OC(C)(C)C)n1]

To 2-((2-(3-((tert-butoxycarbonyl)(6-methoxy-3-nitropyridin-2-yl)amino)propyl)-4-fluorophenyl)amino)-4-chloro-5-fluorobenzoic acid 93c (466 mg, 0.731 mmol) and KHCO₃ (439 mg, 4.39 mmol) in MeOH (15 mL) was added Na₂S₂O₄ (449 mg, 2.19 mmol) in H₂O (15 mL) and the reaction mixture was stirred for 1 h. The reaction mixture was concentrated under reduced pressure, diluted with MeOH (7.5 mL), H₂O (7.5 mL) and 2-MeTHF (20 mL), and treated with 1M HCl (10 ml, 10.0 mmol) and the reaction mixture stirred for 2 h. 1M LiOH (10 mL) was added, followed by 1M K₂HPO₄ (3 mL) to adjust the pH of the mixture to ˜8. The reaction mixture was diluted with H₂O, extracted with EtOAc (2×), and the combined organic extracts were washed with brine, dried over Na₂SO₄, filtered and concentrated under reduced pressure to afford 2-((2-(3-((3-amino-6-methoxypyridin-2-yl)(tert-butoxycarbonyl)amino)propyl)-4-fluorophenyl)-amino)-4-chloro-5-fluorobenzoic acid 93d (438 mg, 98% yield) as a grey solid. HPLC/MS 1.00 min (B), [M+H]⁺ 563.1. ¹H NMR (CD₃OD, 400 MHz): δ 7.75 (d, J=10.3 Hz, 1H), 7.16-7.26 (m, 2H), 6.93-7.05 (m, 2H), 6.49-6.61 (m, 2H), 3.73 (s, 3H), 3.67 (br t, J=6.4 Hz, 2H), 2.64 (t, J=7.8 Hz, 2H), 1.84 (br s, 2H), 1.33-1.47 (m, 9H).

### Step E: tert-Butyl 3-chloro-2,8-difluoro-15-methoxy-19-oxo-5,10,11,12,18,19-hexa-hydro-13H-dibenzo[f,i]pyrido[2,3-b][1,4,8]triazacyclotridecine-13-carboxylate 93e

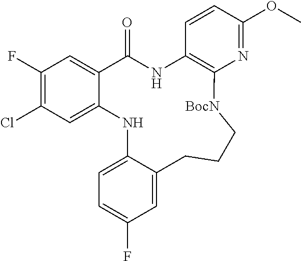 [SMILES: COc1ccc2c(n1)N(C(=O)OC(C)(C)C)CCCc1cc(F)ccc1Nc1cc(Cl)c(F)cc1C(=O)N2]

Following the procedure outlined in Example 27, Step D, stirring the reaction mixture at room temperature for 20 h, tert-butyl 3-chloro-2,8-difluoro-15-methoxy-19-oxo-5,10,11,12,18,19-hexahydro-13H-dibenzo[f,i]pyrido[2,3-b][1,4,8]triazacyclotridecine-13-carboxylate 93e (348 mg, 87% yield) was prepared as a white foamy solid. HPLC/MS 1.54 min (B), [M+H]⁺ 545.1. ¹H NMR (CDCl₃, 400 MHz): δ 8.19 (br d, J=2.0 Hz, 1H), 7.78 (br s, 1H), 7.41 (br s, 1H), 7.27 (br s, 1H), 6.91-7.03 (m, 2H), 6.78 (d, J=8.3 Hz, 1H), 6.59 (br s, 1H), 4.23 (br s, 1H), 3.95-4.07 (m, 1H), 3.92 (s, 3H), 2.59 (br s, 1H), 2.30 (br s, 1H), 1.48-1.68 (m, 2H), 1.31 (s, 9H).

### Step F: tert-Butyl 3-chloro-2,8-difluoro-15-methoxy-19-oxo-11,12-dihydro-19H-5,18-methanodibenzo[f,i]pyrido[2,3-b][1,4,8]triazacyclotridecine-13(10H)-carboxylate 93f

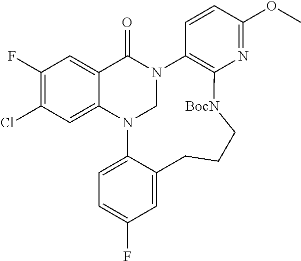 [SMILES: COc1ccc2c(n1)N(C(=O)OC(C)(C)C)CCCc1cc(F)ccc1N1CN2C(=O)c2cc(F)c(Cl)cc21]

Following the procedure outlined in Example 27, Step E, stirring the reaction mixture at 85° C. for 19 h, tert-butyl 3-chloro-2,8-difluoro-15-methoxy-19-oxo-11,12-dihydro-19H-5,18-methanodibenzo[f,i]pyrido[2,3-b][1,4,8]triazacyclotridecine-13(10H)-carboxylate 93f (234 mg, 83% yield) was prepared as a light brown solid. HPLC/MS 1.50 min (B), [M+H]⁺ 557.1. ¹H NMR (CDCl₃, 400 MHz): δ 7.91 (d, J=9.3 Hz, 1H), 7.71 (br s, 1H), 7.23-7.28 (m, 1H), 7.17 (dd, J=8.8, 2.9 Hz, 1H), 7.00 (td, J=8.3, 2.9 Hz, 1H), 6.75 (d, J=8.8 Hz, 1H), 6.57 (br d, J=5.9 Hz, 1H), 5.19 (br d, J=10.3 Hz, 1H), 4.30-4.51 (m, 1H), 3.91 (s, 3H), 3.88 (br s, 1H), 3.36-3.65 (m, 1H), 2.62 (br d, J=8.8 Hz, 1H), 1.97 (br d, J=11.2 Hz, 1H), 1.61 (br dd, J=19.8, 5.6 Hz, 1H), 1.27-1.49 (m, 1H), 1.20 (s, 9H).

### Step G: 3-Chloro-2,8-difluoro-10,11,12,13-tetrahydro-19H-5,18-methanodibenzo[f,i]-pyrido[2,3-b][1,4,8]triazacyclotridecine-15,19(14H)-dione Example 93

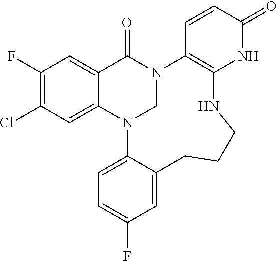 [SMILES: O=C1c2cc(F)c(Cl)cc2N2CN1c1ccc(=O)[nH]c1NCCCc1cc(F)ccc12]

Following the procedure outlined in Example 27, Step F, stirring the reaction mixture at 90° C. for 87 h, 3-chloro-2,8-difluoro-10,11,12,13-tetrahydro-19H-5,18-methanodibenzo[f,i]pyrido-[2,3-b][1,4,8]triazacyclotridecine-15,19(14H)-dione 93 (45 mg, 24% yield) was prepared as an off-white solid. HPLC/MS 1.01 min (B), [M+H]⁺ 443.1. ¹H NMR (CD₃OD, 400 MHz): δ 7.81 (d, J=9.3 Hz, 1H), 7.45 (d, J=9.3 Hz, 1H), 7.41 (dd, J=8.6, 5.1 Hz, 1H), 7.25 (dd, J=9.3, 2.9 Hz, 1H), 7.15 (td, J=8.4, 3.2 Hz, 1H), 6.40 (d, J=5.9 Hz, 1H), 5.93 (d, J=9.3 Hz, 1H), 5.52 (d, J=10.8 Hz, 1H), 4.59 (d, J=10.8 Hz, 1H), 3.35-3.42 (m, 1H), 3.19-3.26 (m, 1H), 2.74 (ddd, J=13.3, 8.9, 3.7 Hz, 1H), 2.54-2.63 (m, 1H), 2.08-2.18 (m, 1H), 1.48-1.58 (m, 1H).

### Example 94

### 3-Chloro-2,8,9-trifluoro-10,11,12,13-tetrahydro-19H-5,18-methanodibenzo[f,i]pyrido[2,3-b][1,4,8]triazacyclotridecine-15,19(14H)-dione

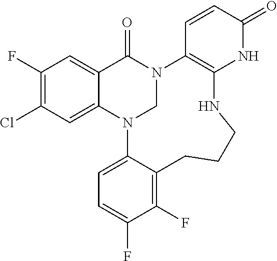 [SMILES: O=C1c2cc(F)c(Cl)cc2N2CN1c1ccc(=O)[nH]c1NCCCc1c2ccc(F)c1F]

### Step A: Methyl 2-((2-(3-((tert-butoxycarbonyl)amino)propyl)-3,4-difluorophenyl)amino)-4-chloro-5-fluorobenzoate 94a

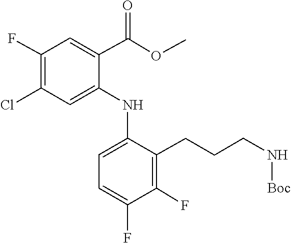 [SMILES: COC(=O)c1cc(F)c(Cl)cc1Nc1ccc(F)c(F)c1CCCNC(=O)OC(C)(C)C]

Following the procedure outlined in Example 7, Step A, substituting methyl 2-bromo-5-(trifluoromethyl)benzoate with methyl 2-bromo-4-chloro-5-fluorobenzoate, Int-3a with Int-3i, BINAP with xantphos, toluene with 1,4-dioxane, and stirring the reaction mixture at 80° C. for 16 h, methyl 2-((2-(3-((tert-butoxycarbonyl)amino)propyl)-3,4-difluorophenyl)amino)-4-chloro-5-fluorobenzoate 94a (273 mg, 701% yield) was prepared as a light brown solid. HPLC/MS 1.56 min (B), [M+H]⁺ 471.1. ¹H NMR (CDCl₃, 400 MHz): δ 9.17 (s, 1H), 7.75 (d, J=9.8 Hz, 1H), 7.01-7.13 (m, 2H), 6.80 (d, J=6.4 Hz, 1H), 4.71 (br s, 1H), 3.96 (s, 3H), 3.18 (br d, J=5.9 Hz, 2H), 2.71 (td, J=7.8, 2.0 Hz, 2H), 1.71-1.81 (m, 2H), 1.42 (s, 9H).

### Step B: Methyl 2-((2-(3-((tert-butoxycarbonyl)(6-methoxy-3-nitropyridin-2-yl)amino)-propyl)-3,4-difluorophenyl)amino)-4-chloro-5-fluorobenzoate 94b

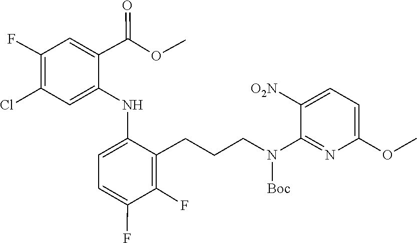 [SMILES: COC(=O)c1cc(F)c(Cl)cc1Nc1ccc(F)c(F)c1CCCN(C(=O)OC(C)(C)C)c1nc(OC)ccc1[N+](=O)[O-]]

Following the procedure outlined in Example 93, Step B, methyl 2-((2-(3-((tert-butoxycarbonyl)(6-methoxy-3-nitropyridin-2-yl)amino)propyl)-3,4-difluorophenyl)amino)-4-chloro-5-fluorobenzoate 94b (175 mg, 45% yield) was prepared as a light brown solid. HPLC/MS 1.69 min (B), [M+H]⁺ 625.1. ¹H NMR (CD₃OD, 400 MHz): δ 9.05 (s, 1H), 8.21 (d, J=8.8 Hz, 1H), 7.69 (d, J=9.8 Hz, 1H), 7.15-7.24 (m, 1H), 7.06 (ddd, J=8.8, 4.4, 1.5 Hz, 1H), 6.67 (d, J=8.8 Hz, 1H), 6.54 (d, J=6.4 Hz, 1H), 3.91 (s, 3H), 3.88 (s, 3H), 3.33 (dt, J=3.1, 1.7 Hz, 2H), 2.80 (br s, 2H), 1.94 (br s, 2H), 1.35 (br s, 9H).

### Step C: 2-((2-(3-((tert-Butoxycarbonyl)(6-methoxy-3-nitropyridin-2-yl)amino)propyl)-3,4-difluorophenyl)amino)-4-chloro-5-fluorobenzoic acid 94c

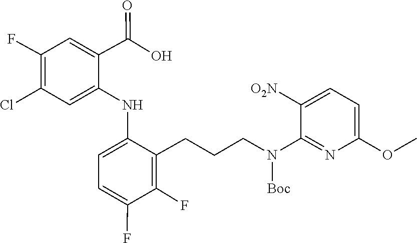 [SMILES: COc1ccc([N+](=O)[O-])c(N(CCCc2c(Nc3cc(Cl)c(F)cc3C(=O)O)ccc(F)c2F)C(=O)OC(C)(C)C)n1]

Following the procedure outlined in Example 27, Step B, stirring the reaction mixture at RT for 5 h, 2-((2-(3-((tert-butoxycarbonyl)(6-methoxy-3-nitropyridin-2-yl)amino)propyl)-3,4-difluorophenyl)amino)-4-chloro-5-fluorobenzoic acid 94c (147 mg, 100% yield) was prepared as a light yellow glass. HPLC/MS 1.14 min (B), [M+H]⁺ 611.0. ¹H NMR (CD₃OD, 400 MHz): δ 8.21 (d, J=8.8 Hz, 1H), 7.71 (d, J=9.8 Hz, 1H), 7.12-7.22 (m, 1H), 7.04-7.10 (m, 1H), 6.66 (d, J=8.8 Hz, 1H), 6.59 (br d, J=4.9 Hz, 1H), 3.92-4.04 (m, 2H), 3.86 (s, 3H), 3.37 (s, 3H), 2.81 (br s, 2H), 1.87-2.03 (m, 2H), 1.35 (br s, 9H).

### Step D: 2-((2-(3-((3-Amino-6-methoxypyridin-2-yl)(tert-butoxycarbonyl)amino)propyl)-3,4-difluorophenyl)amino)-4-chloro-5-fluorobenzoic acid 94d

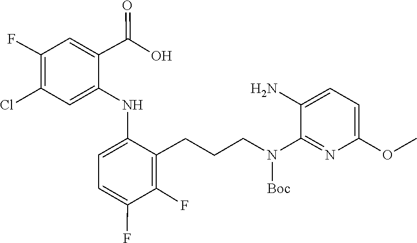 [SMILES: COc1ccc(N)c(N(CCCc2c(Nc3cc(Cl)c(F)cc3C(=O)O)ccc(F)c2F)C(=O)OC(C)(C)C)n1]

Following the procedure outlined in Example 93, Step D, 2-((2-(3-((3-amino-6-methoxypyridin-2-yl)(tert-butoxycarbonyl)amino)propyl)-3,4-difluorophenyl)amino)-4-chloro-5-fluorobenzoic acid 94d (6 mg, 20% yield) was prepared as an off white solid. HPLC/MS 1.06 min (B), [M+H]⁺ 581.1. ¹H NMR (CD₃OD, 400 MHz): δ 7.77 (d, J=10.3 Hz, 1H), 7.07-7.23 (m, 3H), 6.71 (d, J=6.4 Hz, 1H), 6.53 (d, J=8.8 Hz, 1H), 3.72 (s, 3H), 3.65 (br t, J=7.1 Hz, 2H), 2.70-2.77 (m, 2H), 1.86 (br s, 2H), 1.42 (br s, 9H).

### Step E: tert-Butyl 3-chloro-2,8,9-trifluoro-15-methoxy-19-oxo-5,10,11,12,18,19-hexa-hydro-13H-dibenzo[f,i]pyrido[2,3-b][1,4,8]triazacyclotridecine-13-carboxylate 94e

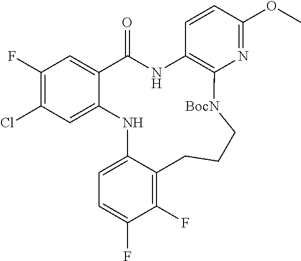 [SMILES: COc1ccc2c(n1)N(C(=O)OC(C)(C)C)CCCc1c(ccc(F)c1F)Nc1cc(Cl)c(F)cc1C(=O)N2]

Following the procedure outlined in Example 27, Step D, stirring the reaction mixture at RT for 17 h, tert-butyl 3-chloro-2,8,9-trifluoro-15-methoxy-19-oxo-5,10,11,12,18,19-hexahydro-13H-dibenzo[f,i]pyrido[2,3-b][1,4,8]triazacyclotridecine-13-carboxylate 94e (4 mg, 81% yield) was prepared as an off white solid. HPLC/MS 1.55 min (B), [M+H]⁺ 563.1. ¹H NMR (CD₃OD, 400 MHz): δ 8.34-8.59 (m, 1H), 7.85 (br d, J=9.3 Hz, 1H), 7.03-7.13 (m, 1H), 6.99 (br dd, J=2.7, 1.2 Hz, 1H), 6.78 (d, J=8.8 Hz, 1H), 6.54-6.72 (m, 1H), 4.06-4.22 (m, 1H), 3.91-4.01 (m, 1H), 3.89 (s, 3H), 2.88 (br s, 2H), 1.91-2.15 (m, 1H), 1.36 (br s, 1H), 1.24 (br s, 9H).

### Step F: tert-Butyl 3-chloro-2,8,9-trifluoro-15-methoxy-19-oxo-11,12-dihydro-19H-5,18-methanodibenzo[f,i]pyrido[2,3-b][1,4,8]triazacyclotridecine-13(10H)-carboxylate 94f

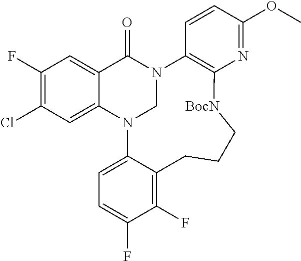 [SMILES: COc1ccc2c(n1)N(C(=O)OC(C)(C)C)CCCc1c(ccc(F)c1F)N1CN2C(=O)c2cc(F)c(Cl)cc21]

Following the procedure outlined in Example 27, Step E, stirring the reaction mixture at 85° C. for 7 h, tert-butyl 3-chloro-2,8,9-trifluoro-15-methoxy-19-oxo-11,12-dihydro-19H-5,18-methanodibenzo[f,i]pyrido[2,3-b][1,4,8]triazacyclotridecine-13(10H)-carboxylate 94f (8 mg, 49% yield) was prepared as a white solid. HPLC/MS 1.53 min (B), [M+H]⁺ 575.1. ¹H NMR (CD₃OD, 400 MHz): δ 7.85 (d, J=9.3 Hz, 1H), 7.80 (br d, J=8.3 Hz, 1H), 7.24-7.32 (m, 2H), 6.85 (d, J=8.8 Hz, 1H), 6.65 (br d, J=4.9 Hz, 1H), 5.29 (br d, J=11.2 Hz, 1H), 4.52 (br s, 1H), 4.07-4.26 (m, 1H), 3.93 (s, 3H), 3.89 (br s, 1H), 2.84-2.97 (m, 1H), 2.00-2.13 (m, 1H), 1.42 (s, 2H), 1.23 (s, 9H).

### Step G: 3-Chloro-2,8,9-trifluoro-10,11,12,13-tetrahydro-19H-5,18-methanodibenzo[f,i]pyrido[2,3-b][1,4,8]triazacyclotridecine-15,19(14H)-dione

### Example 94

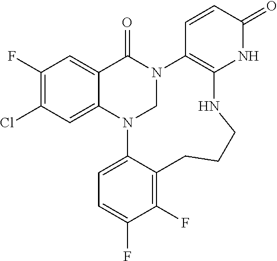 [SMILES: O=C1c2cc(F)c(Cl)cc2N2CN1c1ccc(=O)[nH]c1NCCCc1c2ccc(F)c1F]

Following the procedure outlined in Example 27, Step F, stirring the reaction mixture at 90° C. for 92 h, 3-chloro-2,8,9-trifluoro-10,11,12,13-tetrahydro-19H-5,18-methanodibenzo-[f,i]pyrido[2,3-b][1,4,8]triazacyclotridecine-15,19(14H)-dione 94 (2.2 mg, 34% yield) was prepared. HPLC/MS 1.03 min (B), [M+H]⁺ 461.1. ¹H NMR (CD₃OD, 400 MHz): δ 7.83 (d, J=9.3 Hz, 1H), 7.46 (d, J=9.3 Hz, 1H), 7.24-7.37 (m, 2H), 6.48 (d, J=5.9 Hz, 1H), 5.96 (d, J=8.8 Hz, 1H), 5.47 (d, J=11.2 Hz, 1H), 4.57 (d, J=11.2 Hz, 1H), 3.42-3.49 (m, 1H), 3.18-3.27 (m, 1H), 2.90 (dt, J=13.9, 8.2 Hz, 1H), 2.69-2.78 (m, 1H), 2.11-2.20 (m, 1H), 1.52 (ddt, J=13.6, 9.1, 4.5 Hz, 1H).

### Example 95

### 2,8-Difluoro-15,19-dioxo-10,11,12,13,14,15-hexahydro-19H-5,18-methanodibenzo[f,i]pyrido-[2,3-b][1,4,8]triazacyclotridecine-3-carbonitrile

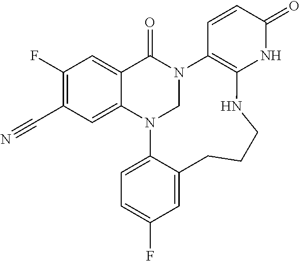 [SMILES: N#Cc1cc2c(cc1F)C(=O)N1CN2c2ccc(F)cc2CCCNc2[nH]c(=O)ccc21]

### Step A: tert-Butyl 3-cyano-2,8-difluoro-15-methoxy-19-oxo-11,12-dihydro-19H-5,18-methanodibenzo[f,i]pyrido[2,3-b][1,4,8]triazacyclotridecine-13(10H)-carboxylate 95a

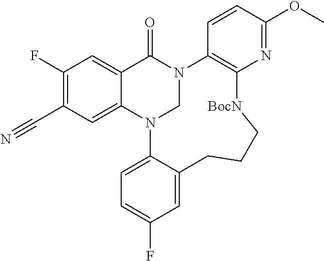 [SMILES: COc1ccc2c(n1)N(C(=O)OC(C)(C)C)CCCc1cc(F)ccc1N1CN2C(=O)c2cc(F)c(C#N)cc21]

Following the procedure outlined in Example 55, Step A, substituting tetrabutylammonium bromide with tetrabutyl ammonium fluoride, using Example 91e as the substrate, and heating at 60° C. for 70 h, tert-butyl 3-cyano-2,8-difluoro-15-methoxy-19-oxo-11,12-dihydro-19H-5,18-methanodibenzo[f,i]pyrido[2,3-b][1,4,8]triazacyclotridecine-13(10H)-carboxylate 95a (294 mg, 87% yield) was prepared as a yellow solid. HPLC/MS 1.31 min (A), [M+H]⁺ 548.2. ¹H NMR (DMSO-d₆, 400 MHz): δ 7.89 (dd, 2H, J=3.4, 8.8 Hz), 7.5-7.5 (m, 1H), 7.4-7.4 (m, 1H), 7.18 (s, 1H), 6.91 (d, 2H, J=8.8 Hz), 5.2-5.4 (m, 1H), 4.4-4.6 (m, 1H), 3.85 (s, 3H), 3.6-3.8 (m, 1H), 3.19 (br d, 1H, J=5.9 Hz), 2.5-2.6 (m, 1H), 1.8-1.9 (m, 1H), 1.3-1.7 (m, 1H), 1.11 (s, 9H).

### Step B: 2,8-Difluoro-15,19-dioxo-10,11,12,13,14,15-hexahydro-19H-5,18-methano-dibenzo[f,i]pyrido[2,3-b][1,4,8]triazacyclotridecine-3-carbonitrile Example 95

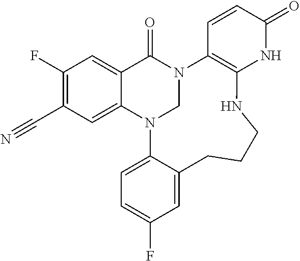 [SMILES: N#Cc1cc2c(cc1F)C(=O)N1CN2c2ccc(F)cc2CCCNc2[nH]c(=O)ccc21]

To tert-butyl 3-cyano-2,8-difluoro-15-methoxy-19-oxo-11,12-dihydro-19H-5,18-methanodibenzo[f,i]pyrido[2,3-b][1,4,8]triazacyclotridecine-13(10H)-carboxylate 95a (65.1 mg, 119 μmol) in DMF (1.30 mL) was added p-TsOH (226 mg, 1.19 mmol) and lithium chloride (50.4 mg, 1.19 mmol), and the reaction mixture was heated at 100° C. for 20 h. The reaction mixture was cooled to ambient temperature and purified by MDAP (XSELECT CSH C18 column, 150 mm×30 mm, 5 uM), eluting with a 70% H₂O-30% MeCN (0.1% FA) to 15% H₂O-85% MeCN (0.1% FA) gradient. Product fractions were combined and lyopholyzed to afford 2,8-difluoro-15,19-dioxo-10,11,12,13,14,15-hexahydro-19H-5,18-methanodibenzo[f,i]pyrido[2,3-b][1,4,8]-triazacyclotridecine-3-carbonitrile (22.5 mg, 42% yield) as a yellow solid. HPLC/MS 0.86 min (A), [M+H]⁺ 434.1. ¹H NMR (CDCl₃, 400 MHz): δ 7.91 (d, 1H, J=8.8 Hz), 7.29 (s, 1H), 7.22 (dd, 1H, J=5.1, 8.6 Hz), 7.0-7.2 (m, 2H), 6.56 (d, 1H, J=4.9 Hz), 5.95 (d, 1H, J=9.3 Hz), 5.46 (d, 1H, J=10.8 Hz), 4.44 (d, 1H, J=10.8 Hz), 3.2-3.3 (m, 2H), 2.5-2.6 (m, 2H), 2.36 (s, 1H), 2.15 (s, 1H), 1.4-1.6 (m, 1H).

### Example 96

### 2-Chloro-3,8-difluoro-10,11,12,13-tetrahydro-19H-5,18-methanodibenzo[f,i]pyrido[2,3-b]-[1,4,8]triazacyclotridecine-15,19(14H)-dione

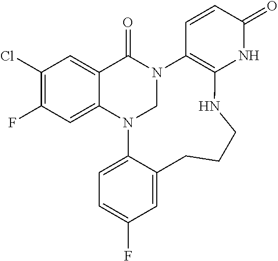 [SMILES: O=C1c2cc(Cl)c(F)cc2N2CN1c1ccc(=O)[nH]c1NCCCc1cc(F)ccc12]

### Step A: tert-Butyl (3-(5-fluoro-2-(2,2,2-trifluoroacetamido)phenyl)prop-2-yn-1-yl)(6-methoxy-3-nitropyridin-2-yl)carbamate 96a

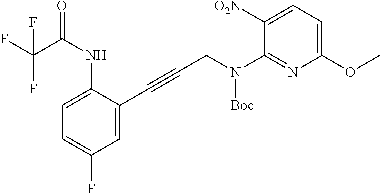 [SMILES: COc1ccc([N+](=O)[O-])c(N(CC#Cc2cc(F)ccc2NC(=O)C(F)(F)F)C(=O)OC(C)(C)C)n1]

To tert-butyl (3-(2-amino-5-fluorophenyl)prop-2-yn-1-yl)(6-methoxy-3-nitropyridin-2-yl)carbamate Int-3e (8.00 g, 19.2 mmol) and TEA (6.69 ml, 48.0 mmol) in 2-MeTHF (96 ml) at 0° C. was added 2,2,2-trifluoroacetic anhydride (8.01 ml, 57.6 mmol). The reaction mixture was allowed to warm to 25° C. over 1 h, then partitioned between H₂O and EtOAc (10 mL) and the layers separated. The organic layer was pre-loaded onto silica gel and purified silica gel flash column chromatography (40 g), eluting with a 100% heptanes to 20% EtOAc-heptanes gradient. Product fractions were combined and evaporated under reduced pressure to afford tert-butyl (3-(5-fluoro-2-(2,2,2-trifluoroacetamido)phenyl)prop-2-yn-1-yl)(6-methoxy-3-nitropyridin-2-yl)-carbamate 96a (9.31 g, 95% yield) as a light yellow solid. HPLC/MS 1.35 min (B), [M+H]⁺ 513.0. ¹H NMR (DMSO-d₆, 400 MHz): δ 11.02 (br s, 1H), 8.38 (d, J=8.8 Hz, 1H), 7.39-7.46 (m, 1H), 7.27-7.36 (m, 2H), 6.86 (d, J=8.8 Hz, 1H), 4.92 (s, 2H), 3.98 (s, 3H), 1.36 (br s, 9H).

### Step B: tert-Butyl (3-amino-6-methoxypyridin-2-yl)(3-(5-fluoro-2-(2,2,2-trifluoro-acetamido)phenyl)propyl)carbamate 96b

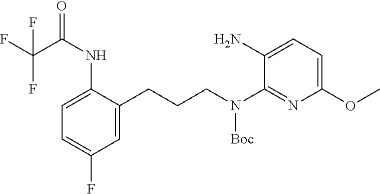 [SMILES: COc1ccc(N)c(N(CCCc2cc(F)ccc2NC(=O)C(F)(F)F)C(=O)OC(C)(C)C)n1]

A mixture of tert-butyl (3-(5-fluoro-2-(2,2,2-trifluoroacetamido)phenyl)prop-2-yn-1-yl)(6-methoxy-3-nitropyridin-2-yl)carbamate 96a (9.31 g, 18.2 mmol) and Pd(OH)₂ (4.46 g, 31.8 mmol) in ethanol (20.0 mL) and EtOAc (20.0 mL) at 25° C. was stirred under a balloon of hydrogen. After 24 h, the reaction mixture was filtered thru a glass filter and evaporated under reduced pressure to afford tert-butyl (3-amino-6-methoxypyridin-2-yl)(3-(5-fluoro-2-(2,2,2-trifluoroacetamido) phenyl)propyl)carbamate 96b (8.77 g, 91% yield) as a light red foam. HPLC/MS 1.16 min (B), [M+H]⁺ 487.2. ¹H NMR (DMSO-d₆, 400 MHz): δ 10.91 (s, 1H), 7.28 (dd, J=8.3, 5.4 Hz, 1H), 7.21 (br d, J=8.3 Hz, 1H), 7.05-7.13 (m, 2H), 6.57 (d, J=8.3 Hz, 1H), 3.68 (s, 3H), 3.56 (br s, 2H), 2.52-2.57 (m, 2H), 1.66-1.79 (m, 2H), 1.38 (br s, 9H).

### Step C: tert-Butyl (3-(2-bromo-5-chloro-4-fluorobenzamido)-6-methoxypyridin-2-yl)(3-(5-fluoro-2-(2,2,2-trifluoroacetamido)phenyl)propyl)carbamate 96c

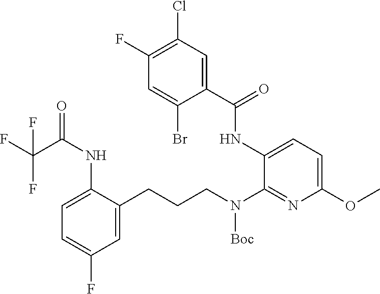 [SMILES: COc1ccc(NC(=O)c2cc(Cl)c(F)cc2Br)c(N(CCCc2cc(F)ccc2NC(=O)C(F)(F)F)C(=O)OC(C)(C)C)n1]

To tert-butyl (3-amino-6-methoxypyridin-2-yl)(3-(5-fluoro-2-(2,2,2-trifluoroacetamido)-phenyl)propyl)carbamate 96b (1.00 g, 2.06 mmol), HATU (1.02 g, 2.67 mmol) and DIEA (1.07 mL, 6.17 mmol) in DMF (10.0 mL) at 25° C. was added 2-bromo-5-chloro-4-fluorobenzoic acid (573.1 mg, 2.26 mmol) and the reaction mixture was stirred for 18 h. The reaction mixture was quenched with H₂O (50 mL), the resulting tan solid collected by filtration, and purified by silica gel flash column chromatography (40 g), eluting with a 100% heptanes to 40% EtOAc-heptanes gradient. Pure product fractions were combined and evaporated under reduced pressure to afford tert-butyl (3-(2-bromo-5-chloro-4-fluorobenzamido)-6-methoxypyridin-2-yl)(3-(5-fluoro-2-(2,2,2-trifluoroacetamido)phenyl)propyl)carbamate 96c (1.15 g, 78% yield) as a white solid. HPLC/MS 1.47 min (B), [M+H]⁺ 723.0. ¹H NMR (DMSO-d₆, 400 MHz): δ 10.93 (s, 1H), 9.66-9.92 (m, 1H), 8.01 (br s, 1H), 7.96 (d, J=8.8 Hz, 1H), 7.72 (d, J=7.3 Hz, 1H), 7.29 (dd, J=8.3, 5.4 Hz, 1H), 7.06-7.16 (m, 2H), 6.84 (d, J=8.8 Hz, 1H), 3.80 (s, 3H), 3.44-3.72 (m, 2H), 2.56 (t, J=7.6 Hz, 2H), 1.91 (br d, J=2.9 Hz, 2H), 1.36 (br s, 9H).

### Step D: tert-Butyl (3-(2-amino-5-fluorophenyl)propyl)(3-(2-bromo-5-chloro-4-fluorobenzamido)-6-methoxypyridin-2-yl)carbamate 96d

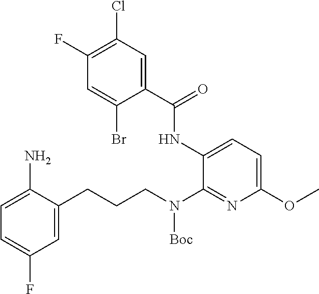 [SMILES: COc1ccc(NC(=O)c2cc(Cl)c(F)cc2Br)c(N(CCCc2cc(F)ccc2N)C(=O)OC(C)(C)C)n1]

A suspension of tert-butyl (3-(2-bromo-5-chloro-4-fluorobenzamido)-6-methoxypyridin-2-yl)(3-(5-fluoro-2-(2,2,2-trifluoroacetamido)phenyl)propyl)carbamate 96c (1.15 g, 1.59 mmol) and NaBH₄ (301.3 mg, 7.97 mmol) in THF (10.0 mL) was stirred at 25° C. for 2 d, then partitioned between EtOAc and H₂O. The organic layer was dried over MgSO₄, filtered and concentrated under reduced pressure. The crude residue was purified by silica gel flash column chromatography (40 g), eluting with a 100% hepatanes to 40% EtOAc-heptanes gradient. Pure product fractions were combined and evaporated under reduced pressure to afford tert-butyl (3-(2-amino-5-fluorophenyl)propyl)(3-(2-bromo-5-chloro-4-fluorobenzamido)-6-methoxypyridin-2-yl)-carbamate 96d (137 mg, 13.% yield). HPLC/MS 1.43 min (B), [M+H]⁺ 627.1. ¹H NMR (DMSO-d₆, 400 MHz): δ 9.5-10.0 (m, 1H), 8.0-8.1 (m, 1H), 7.96 (d, 1H, J=9.3 Hz), 7.7-7.7 (m, 1H), 6.83 (d, 1H, J=8.8 Hz), 6.69 (d, 2H, J=9.3 Hz), 6.5-6.6 (m, 1H), 4.69 (s, 2H), 3.82 (s, 3H), 3.4-3.7 (m, 2H), 2.4-2.5 (m, 2H), 1.8-2.0 (m, 2H), 1.3-1.5 (m, 9H).

### Step E: tert-Butyl 2-chloro-3,8-difluoro-15-methoxy-19-oxo-5,10,11,12,18,19-hexa-hydro-13H-dibenzo[f,i]pyrido[2,3-b][1,4,8]triazacyclotridecine-13-carboxylate 96e

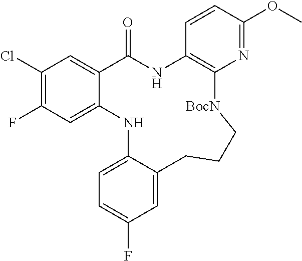 [SMILES: COc1ccc2c(n1)N(C(=O)OC(C)(C)C)CCCc1cc(F)ccc1Nc1cc(F)c(Cl)cc1C(=O)N2]

A suspension of tert-butyl (3-(2-amino-5-fluorophenyl)propyl)(3-(2-bromo-5-chloro-4-fluorobenzamido)-6-methoxypyridin-2-yl)carbamate 96d (137.0 mg, 218.9 μmol), copper(I) iodide (62.5 mg, 328.3 μmol) and DIEA (876 μmol) in DMF (10.0 mL) was purged with N₂ for 5 min, heated at 95° C. for 36 h, stirred for 36 h at 25° C. and poured over ice. The resulting solid was collected by filtration, dried under vacuo and purified by silica gel flash column chromatography (24 g), eluting with a 100% heptanes to 50% EtOAc-heptanes gradient. Pure product fractions were combined and evaporated under reduced pressure to afford tert-butyl 2-chloro-3,8-difluoro-15-methoxy-19-oxo-5,10,11,12,18,19-hexahydro-13H-dibenzo[f,i]pyrido-[2,3-b][1,4,8]triazacyclotridecine-13-carboxylate 96e (35 mg, 29% yield) as a white solid. HPLC/MS 1.52 min (B), [M+H]⁺ 545.1. ¹H NMR (CDCl₃, 400 MHz): δ 8.10 (s, 1H), 7.82 (br d, J=4.9 Hz, 1H), 7.69-7.78 (m, 1H), 7.64 (d, J=7.8 Hz, 1H), 6.93-7.06 (m, 2H), 6.79 (d, J=8.3 Hz, 1H), 6.32 (br d, J=11.2 Hz, 1H), 4.22-4.36 (m, 1H), 3.94-4.04 (m, 1H), 3.92 (s, 3H), 2.53-2.66 (m, 1H), 2.27 (br s, 1H), 1.60 (br d, J=4.9 Hz, 1H), 1.49-1.55 (m, 1H), 1.33 (s, 9H).

### Step F: tert-Butyl 2-chloro-3,8-difluoro-15-methoxy-19-oxo-11,12-dihydro-19H-5,18-methanodibenzo[f,i]pyrido[2,3-b][1,4,8]triazacyclotridecine-13(10H)-carboxylate 96f

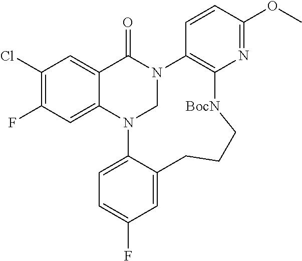 [SMILES: COc1ccc2c(n1)N(C(=O)OC(C)(C)C)CCCc1cc(F)ccc1N1CN2C(=O)c2cc(Cl)c(F)cc21]

Following the procedure outlined in Example 27, Step E, stirring the reaction mixture at 95° C. for 18 h, tert-butyl 2-chloro-3,8-difluoro-15-methoxy-19-oxo-11,12-dihydro-19H-5,18-methanodibenzo[f,i]pyrido[2,3-b][1,4,8]triazacyclotridecine-13(10H)-carboxylate 96f (20 mg, 55% yield) was prepared. HPLC/MS 1.47 min (B), [M+H]⁺ 557.1. ¹H NMR (CDCl₃, 400 MHz): δ 8.19 (d, 1H, J=7.8 Hz), 7.72 (br d, 1H, J=4.9 Hz), 7.25 (dd, 1H, J=5.4, 8.8 Hz), 7.17 (dd, 1H, J=2.9, 9.3 Hz), 7.00 (dt, 1H, J=2.9, 8.1 Hz), 6.6-6.8 (m, 1H), 6.31 (br d, 1H, J=10.8 Hz), 4.2-5.3 (m, 2H), 3.9-4.0 (m, 3H), 3.8-3.9 (m, 2H), 2.4-2.8 (m, 2H), 1.8-2.1 (m, 2H), 1.58 (s, 9H).

### Step G: 2-Chloro-3,8-difluoro-10,11,12,13-tetrahydro-19H-5,18-methanodibenzo[f,i]-pyrido[2,3-b][1,4,8]triazacyclotridecine-15,19(14H)-dione Example 96

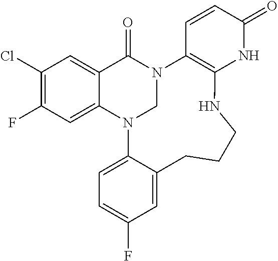 [SMILES: O=C1c2cc(Cl)c(F)cc2N2CN1c1ccc(=O)[nH]c1NCCCc1cc(F)ccc12]

Following the procedure outlined in Example 27, Step F, stirring the reaction mixture at 90° C. for 6 days, 2-chloro-3,8-difluoro-10,11,12,13-tetrahydro-19H-5,18-methanodibenzo[f,i]-pyrido[2,3-b][1,4,8]triazacyclotridecine-15,19(14H)-dione 96 (5.0 mg, 25% yield) was prepared as a white solid. HPLC/MS 1.01 min (B), [M+H]⁺ 443.1. ¹H NMR (CDCl₃, 400 MHz): δ 8.16 (d, J=8.3 Hz, 1H), 7.41 (br d, J=7.8 Hz, 1H), 7.20 (dd, J=8.6, 5.1 Hz, 1H), 7.05-7.16 (m, 2H), 6.13 (d, J=10.3 Hz, 1H), 5.96-6.04 (m, 1H), 5.45 (br d, J=10.8 Hz, 1H), 5.14-5.25 (m, 1H), 4.36 (br d, J=11.2 Hz, 1H), 3.29-3.43 (m, 2H), 2.57-2.74 (m, 3H), 2.20 (br dd, J=7.8, 4.9 Hz, 1H), 1.55-1.65 (m, 1H).

### Example 97

### 1,8,9-Trifluoro-2-(trifluoromethyl)-10,11,12,13-tetrahydro-19H-5,18-methanodibenzo[f,i]pyrido-[2,3-b][1,4,8]triazacyclotridecine-15,19(14H)-dione

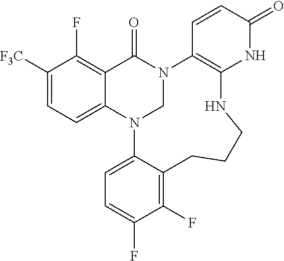 [SMILES: O=C1c2c(ccc(C(F)(F)F)c2F)N2CN1c1ccc(=O)[nH]c1NCCCc1c2ccc(F)c1F]

### Step A: Methyl 6-((2-(3-((tert-butoxycarbonyl)amino)propyl)-3,4-difluorophenyl)-amino)-2-fluoro-3-(trifluoromethyl)benzoate 97a

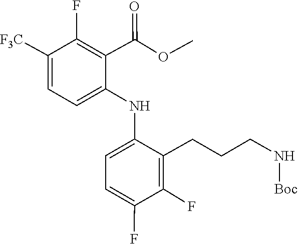 [SMILES: COC(=O)c1c(Nc2ccc(F)c(F)c2CCCNC(=O)OC(C)(C)C)ccc(C(F)(F)F)c1F]

Following the procedure outlined in Example 7, Step A, substituting methyl 2-bromo-5-(trifluoromethyl)benzoate with methyl 6-bromo-2-fluoro-3-(trifluoromethyl)benzoate, Int-3a with Int-3i, BINAP with xantphos, toluene with 1,4-dioxane, and stirring the reaction mixture at 80° C. for 18 h, methyl 6-((2-(3-((tert-butoxycarbonyl)amino)propyl)-3,4-difluorophenyl)amino)-2-fluoro-3-(trifluoromethyl)benzoate 97a (2.09 g, 73% yield) was prepared as a white solid. HPLC/MS 1.50 min (B), [M−H]⁻ 505.1. ¹H NMR (CD₃OD, 400 MHz): δ 1.39 (s, 9H) 1.68-1.78 (m, 2H), 2.57-2.65 (m, 2H), 3.01-3.09 (m, 2H), 3.99 (s, 3H), 6.67-6.76 (m, 1H), 7.01-7.09 (m, 1H), 7.12-7.20 (m, 1H), 7.25-7.33 (m, 1H), 7.48-7.56 (m, 1H), 8.22 (d, J=1.47 Hz, 1H), 9.52-9.61 (m, 1H).

### Step B: Methyl 6-((2-(3-((tert-butoxycarbonyl)(6-methoxy-3-nitropyridin-2-yl)amino)-propyl)-3,4-difluorophenyl)amino)-2-fluoro-3-(trifluoromethyl)benzoate 97b

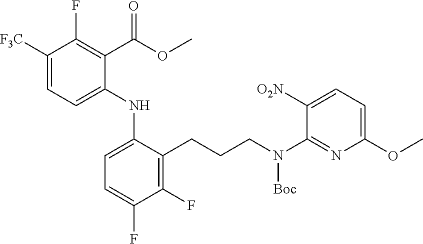 [SMILES: COC(=O)c1c(Nc2ccc(F)c(F)c2CCCN(C(=O)OC(C)(C)C)c2nc(OC)ccc2[N+](=O)[O-])ccc(C(F)(F)F)c1F]

Following the procedure outlined in Example 93, Step B, methyl 6-((2-(3-((tert-butoxycarbonyl)(6-methoxy-3-nitropyridin-2-yl)amino)propyl)-3,4-difluorophenyl)amino)-2-fluoro-3-(trifluoromethyl)benzoate 97b (1.45 g, 76% yield) was prepared as an off-white solid. HPLC/MS 1.68 min (B), [M+H]⁺ 659.0. ¹H NMR (CD₃OD, 400 MHz): δ 1.27-1.44 (m, 9H), 1.82-2.01 (m, 2H), 2.71-2.88 (m, 2H), 3.89 (s, 3H), 3.96 (s, 3H), 3.98-4.12 (m, 2H), 6.36 (d, J=8.80 Hz, 1H), 6.67 (d, J=8.80 Hz, 1H), 7.06 (ddd, J=8.93, 4.52, 1.71 Hz, 1H), 7.16-7.26 (m, 1H), 7.41 (t, J=8.56 Hz, 1H), 8.20 (d, J=8.80 Hz, 1H).

### Step C: 6-((2-(3-((tert-butoxycarbonyl)(6-methoxy-3-nitropyridin-2-yl)amino)propyl)-3,4-difluorophenyl)amino)-2-fluoro-3-(trifluoromethyl)benzoic acid 97c

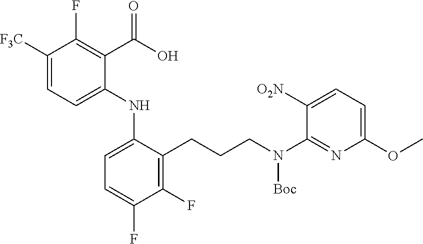 [SMILES: COc1ccc([N+](=O)[O-])c(N(CCCc2c(Nc3ccc(C(F)(F)F)c(F)c3C(=O)O)ccc(F)c2F)C(=O)OC(C)(C)C)n1]

Following the procedure outlined in Example 27, Step B, stirring the reaction mixture at RT for 18 h, 6-((2-(3-((tert-butoxycarbonyl)(6-methoxy-3-nitropyridin-2-yl)amino)propyl)-3,4-difluorophenyl)amino)-2-fluoro-3-(trifluoromethyl)benzoic acid 97c (1.61 g, 100% yield) was prepared as a light yellow glass. HPLC/MS 1.17 min (B), [M+H]⁺ 645.1. ¹H NMR (CD₃OD, 400 MHz): δ 1.35 (br s, 9H), 1.86-2.07 (m, 2H), 2.72-2.88 (m, 2H), 3.87 (s, 3H), 3.95-4.10 (m, 2H), 6.38 (br d, J=8.80 Hz, 1H), 6.67 (d, J=8.80 Hz, 1H), 7.03-7.11 (m, 1H), 7.21 (d, J=9.78 Hz, 1H), 7.40 (t, J=8.31 Hz, 1H), 8.21 (d, J=9.29 Hz, 1H).

### Step D: 6-((2-(3-((3-Amino-6-methoxypyridin-2-yl)(tert-butoxycarbonyl)amino)propyl)-3,4-difluorophenyl)amino)-2-fluoro-3-(trifluoromethyl)benzoic acid 97d

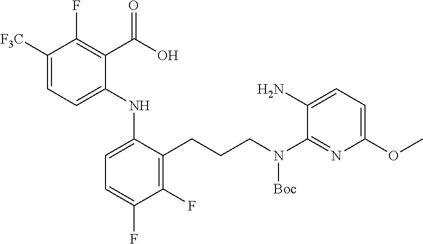 [SMILES: COc1ccc(N)c(N(CCCc2c(Nc3ccc(C(F)(F)F)c(F)c3C(=O)O)ccc(F)c2F)C(=O)OC(C)(C)C)n1]

Following the procedure outlined in Example 93, Step D, 6-((2-(3-((3-amino-6-methoxypyridin-2-yl)(tert-butoxycarbonyl)amino)propyl)-3,4-difluorophenyl)amino)-2-fluoro-3-(trifluoromethyl)benzoic acid 97d (967 mg, 100% yield) was prepared as a light brown foam. HPLC/MS 1.06 min (B), [M+H]⁺ 615.15. ¹H NMR (CD₃OD, 400 MHz): δ 1.41 (br s, 9H), 1.77-1.97 (m, 2H), 2.70-2.79 (m, 2H), 3.60-3.69 (m, 2H), 3.71 (s, 3H), 6.43-6.48 (m, 1H), 6.52 (d, J=8.31 Hz, 1H), 7.04-7.22 (m, 3H), 7.25-7.32 (m, 1H).

### Step E: tert-Butyl 1,8,9-trifluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 97e

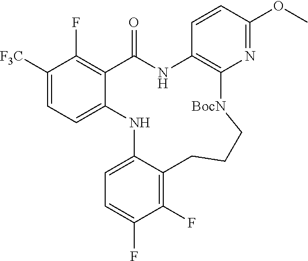 [SMILES: COc1ccc2c(n1)N(C(=O)OC(C)(C)C)CCCc1c(ccc(F)c1F)Nc1ccc(C(F)(F)F)c(F)c1C(=O)N2]

Following the procedure outlined in Example 27, Step D, stirring the reaction mixture at room temperature for 1 h, tert-butyl 1,8,9-trifluoro-15-methoxy-19-oxo-2-(trifluoromethyl)-5,10,11,12,18,19-hexahydro-13H-dibenzo[f,i]pyrido[2,3-b][1,4,8]triazacyclotridecine-13-carboxylate 97e (809 mg, 90% yield) was prepared as a light brown solid. HPLC/MS 1.62 min (B), [M+H]⁺ 597.17. ¹H NMR (CDCl₃, 400 MHz): δ 1.29 (s, 9H), 1.35-1.50 (m, 2H), 2.25-2.40 (m, 1H), 2.56-2.74 (m, 1H), 3.90 (s, 3H), 3.95-4.07 (m, 1H), 4.24-4.41 (m, 1H), 6.31 (br d, J=8.31 Hz, 1H), 6.78 (d, J=8.31 Hz, 1H), 7.04-7.11 (m, 2H), 7.34 (s, 1H), 7.68-7.77 (m, 1H), 7.78-7.88 (m, 1H), 8.24-8.36 (m, 1H).

### Step F: tert-Butyl 1,8,9-trifluoro-15-methoxy-19-oxo-2-(trifluoromethyl)-11,12-dihydro-19H-5,18-methanodibenzo[f,i]pyrido[2,3-b][1,4,8]triazacyclotridecine-13(10H)-carboxylate 97f

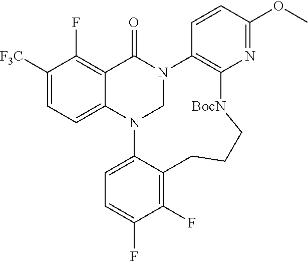 [SMILES: COc1ccc2c(n1)N(C(=O)OC(C)(C)C)CCCc1c(ccc(F)c1F)N1CN2C(=O)c2c1ccc(C(F)(F)F)c2F]

Following the procedure outlined in Example 27, Step E, stirring the reaction mixture at 90° C. for 17 h, tert-butyl 1,8,9-trifluoro-15-methoxy-19-oxo-2-(trifluoromethyl)-11,12-dihydro-19H-5,18-methanodibenzo[f,i]pyrido[2,3-b][1,4,8]triazacyclotridecine-13(10H)-carboxylate 97f (175 mg, 21% yield) was prepared as a light brown solid. HPLC/MS 1.50 min (B), [M+H]⁺ 609.09. ¹H NMR (CDCl₃, 400 MHz): δ 1.21 (br s, 9H), 2.06-2.27 (m, 1H), 2.82-3.07 (m, 1H), 3.14-3.45 (m, 1H), 3.91 (br s, 4H), 4.25-4.59 (m, 1H), 5.07-5.39 (m, 1H), 6.23-6.41 (m, 1H), 6.76 (d, J=8.80 Hz, 1H), 6.94-7.06 (m, 1H), 7.14 (d, J=9.29 Hz, 1H), 7.40-7.52 (m, 1H), 7.62-7.89 (m, 1H).

### Step G: 1,8,9-Trifluoro-2-(trifluoromethyl)-10,11,12,13-tetrahydro-19H-5,18-methanodibenzo[f,i]pyrido[2,3-b][1,4,8]triazacyclotridecine-15,19(14H)-dione

### Example 97

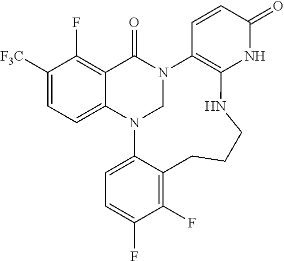 [SMILES: O=C1c2c(ccc(C(F)(F)F)c2F)N2CN1c1ccc(=O)[nH]c1NCCCc1c2ccc(F)c1F]

Following the procedure outlined in Example 27, Step F, stirring the reaction mixture at 90° C. for 92 h, 1,8,9-trifluoro-2-(trifluoromethyl)-10,11,12,13-tetrahydro-19H-5,18-methano-dibenzo[f,i]pyrido[2,3-b][1,4,8]triazacyclotridecine-15,19(14H)-dione (2.2 mg, 34% yield) was prepared as an off-white solid. HPLC/MS 1.02 min (B), [M+H]⁺ 495.14. ¹H NMR (CD₃OD, 400 MHz): δ 1.44-1.60 (m, 1H), 2.07-2.23 (m, 1H), 2.66-2.79 (m, 1H), 2.83-2.96 (m, 1H), 3.23 (s, 1H), 3.44 (s, 1H), 4.65 (d, J=11.25 Hz, 1H), 5.50 (d, J=11.74 Hz, 1H), 5.99 (d, J=9.29 Hz, 1H), 6.32 (d, J=8.80 Hz, 1H), 7.20-7.26 (m, 1H), 7.29-7.38 (m, 1H), 7.49 (d, J=9.29 Hz, 1H), 7.62 (dd, J=8.56, 7.58 Hz, 1H).

### Example 98

### 2,3,8,9-Tetrafluoro-10,11,12,13-tetrahydro-19H-5,18-methanodibenzo[f,i]pyrido[2,3-b]-[1,4,8]triazacyclotridecine-15,19(14H)-dione, formic acid salt

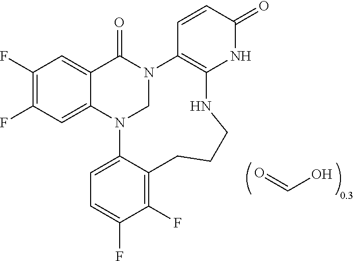

### Step A: Methyl 2-((2-(3-((tert-butoxycarbonyl)(6-methoxy-3-nitropyridin-2-yl)amino)-prop-1-yn-1-yl)-3,4-difluorophenyl)amino)-4,5-difluorobenzoate 98a

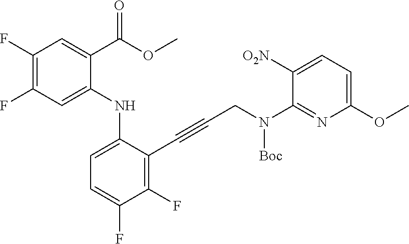 [SMILES: COC(=O)c1cc(F)c(F)cc1Nc1ccc(F)c(F)c1C#CCN(C(=O)OC(C)(C)C)c1nc(OC)ccc1[N+](=O)[O-]]

Following the procedure outlined in Example 27, Step A, substituting methyl 2-bromo-5-(trifluoromethyl)benzoate with methyl 2-bromo-4,5-difluorobenzoate, Int-3e with Int-3m, palladium(II) acetate with tris(dibezylideneacetone)dipalladium, and stirring the reaction at 100° C. for 5 h, methyl 2-((2-(3-((tert-butoxycarbonyl)(6-methoxy-3-nitropyridin-2-yl)amino)prop-1-yn-1-yl)-3,4-difluorophenyl)amino)-4,5-difluorobenzoate 98a (1.05 g, 79% yield) was prepared. HPLC/MS 1.55 min (A), [M+H]⁺ 605. ¹H NMR (DMSO-d₆, 400 MHz): δ 9.44 (br s, 1H), 8.32 (d, 1H, J=8.8 Hz), 7.80 (dd, 1H, J=9.0, 11.5 Hz), 7.45 (q, 1H, J=9.3 Hz), 7.2-7.3 (m, 1H), 7.11 (dd, 1H, J=6.8, 13.2 Hz), 6.81 (d, 1H, J=8.8 Hz), 4.97 (s, 2H), 3.88 (s, 3H), 3.76 (s, 3H), 1.2-1.4 (m, 9H).

### Step B: Methyl 2-((2-(3-((3-amino-6-methoxypyridin-2-yl)(tert-butoxycarbonyl)amino)-propyl)-3,4-difluorophenyl)amino)-4,5-difluorobenzoate 98b

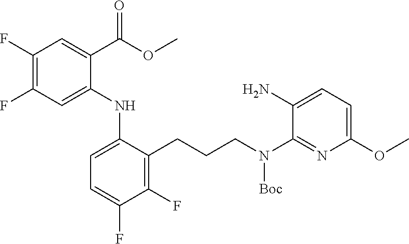 [SMILES: COC(=O)c1cc(F)c(F)cc1Nc1ccc(F)c(F)c1CCCN(C(=O)OC(C)(C)C)c1nc(OC)ccc1N]

Following the procedure outlined in Example 27, Step C, using an EtOH: EtOAc (1.7:1) mixture as solvent and stirring the reaction mixture at room temperature for 5 h, methyl 2-((2-(3-((3-amino-6-methoxypyridin-2-yl)(tert-butoxycarbonyl)amino)propyl)-3,4-difluoro-phenyl)-amino)-4,5-difluorobenzoate 98b (0.999 g, 80% yield) was prepared. HPLC/MS 1.33 min (A), [M+H]⁺ 579. ¹H NMR (DMSO-d₆, 400 MHz): δ 9.13 (s, 1H), 7.82 (dd, 1H, J=9.0, 11.5 Hz), 7.3-7.4 (m, 1H), 7.1-7.2 (m, 2H), 6.56 (dd, 1H, J=7.1, 13.4 Hz), 6.50 (d, 1H, J=8.3 Hz), 4.3-4.4 (m, 3H), 3.85 (s, 3H), 3.60 (s, 2H), 3.45 (br range, 2H), 2.62 (br t, 2H, J=7.3 Hz), 1.72 (br s, 2H), 1.32 (s, 9H).

### Step C: 2-((2-(3-((3-Amino-6-methoxypyridin-2-yl)(tert-butoxycarbonyl)amino)propyl)-3,4-difluorophenyl)amino)-4,5-difluorobenzoic acid, dihydrochloride 98c

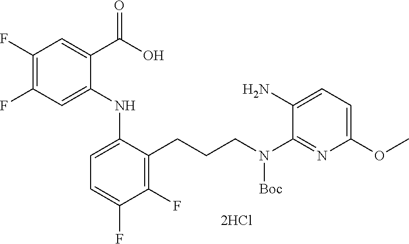 [SMILES: COc1ccc(N)c(N(CCCc2c(Nc3cc(F)c(F)cc3C(=O)O)ccc(F)c2F)C(=O)OC(C)(C)C)n1]

Following the procedure outlined in Example 27, Step B, stirring the reaction mixture at RT for 2 h, 2-((2-(3-((3-amino-6-methoxypyridin-2-yl)(tert-butoxycarbonyl)amino)propyl)-3,4-difluorophenyl)amino)-4,5-difluorobenzoic acid, dihydrochloride 98c (0.966 g, 100% yield) was prepared. HPLC/MS 1.21 min (A), [M+H]⁺ 565. ¹H NMR (DMSO-d₆, 400 MHz): δ 9.49 (s, 1H), 7.81 (dd, 1H, J=9.3, 11.2 Hz), 7.3-7.4 (m, 1H), 7.18 (br dd, 2H, J=4.4, 8.8 Hz), 6.58 (dd, 1H, J=6.8, 13.7 Hz), 6.52 (br d, 1H, J=8.3 Hz), 3.62 (s, 3H), 3.4-3.6 (m, 2H), 2.64 (br t, 2H, J=7.3 Hz), 1.76 (br d, 2H, J=4.9 Hz), 1.32 (s, 9H).

### Step D: tert-Butyl 2,3,8,9-tetrafluoro-15-methoxy-19-oxo-5,10,11,12,18,19-hexahydro-13H-dibenzo[f,i]pyrido[2,3-b][1,4,8]triazacyclotridecine-13-carboxylate 98d

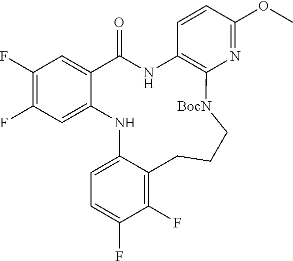 [SMILES: COc1ccc2c(n1)N(C(=O)OC(C)(C)C)CCCc1c(ccc(F)c1F)Nc1cc(F)c(F)cc1C(=O)N2]

Following the procedure outlined in Example 27, Step D, stirring the reaction mixture at room temperature for 2 h, tert-butyl 2,3,8,9-tetrafluoro-15-methoxy-19-oxo-5,10,11,12,18,19-hexahydro-13H-dibenzo[f,i]pyrido[2,3-b][1,4,8]triazacyclotridecine-13-carboxylate 98d (0.566 g, 79% yield) was prepared. HPLC/MS 1.44 min (A), [M+H]⁺ 547. ¹H NMR (DMSO-d₆, 501 MHz): δ 9.58 (br s, 1H), 8.42 (br d, J=8.9 Hz, 1H), 7.86-7.95 (m, 2H), 7.13-7.22 (m, 1H), 6.79-6.87 (m, 2H), 6.74 (br dd, J=7.8, 3.2 Hz, 1H), 3.91 (br s, 2H), 3.84 (s, 3H), 2.79 (br t, J=7.8 Hz, 2H), 1.46-1.74 (m, 2H), 1.20 (s, 9H).

### Step E: tert-Butyl 2,3,8,9-tetrafluoro-15-methoxy-19-oxo-11,12-dihydro-19H-5,18-methanodibenzo[f,i]pyrido[2,3-b][1,4,8]triazacyclotridecine-13(10H)-carboxylate 98e

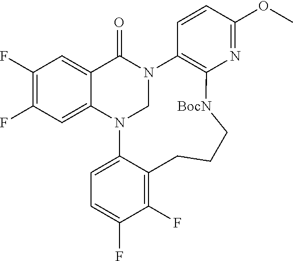 [SMILES: COc1ccc2c(n1)N(C(=O)OC(C)(C)C)CCCc1c(ccc(F)c1F)N1CN2C(=O)c2cc(F)c(F)cc21]

Following the procedure outlined in Example 27, Step E, stirring the reaction mixture at 90° C. until complete, tert-butyl 2,3,8,9-tetrafluoro-15-methoxy-19-oxo-11,12-dihydro-19H-5,18-methanodibenzo[f,i]pyrido[2,3-b][1,4,8]triazacyclotridecine-13(10H)-carboxylate 98e (578.4 g, 79% yield) was prepared. HPLC/MS 1.44 min (A), [M+H]⁺ 547. ¹H NMR (DMSO-d₆, 400 MHz): δ 7.8-7.9 (m, 2H), 7.4-7.5 (m, 1H), 7.3-7.4 (m, 1H), 6.89 (d, 1H, J=8.3 Hz), 6.5-6.6 (m, 1H), 5.28 (br d, 1H, J=11.7 Hz), 4.49 (br d, 1H, J=10.8 Hz), 3.9-4.2 (m, 1H), 3.85 (s, 3H), 3.72 (br d, 1H, J=12.7 Hz), 3.1-3.2 (m, 1H), 2.7-2.8 (m, 1H), 1.9-2.0 (m, 1H), 1.4-1.6 (m, 1H), 1.11 (s, 9H).

### Step F: 2,3,8,9-Tetrafluoro-10,11,12,13-tetrahydro-19H-5,18-methanodibenzo[f,i]-pyrido[2,3-b][1,4,8]triazacyclotridecine-15,19(14H)-dione, formic acid salt

### Example 98

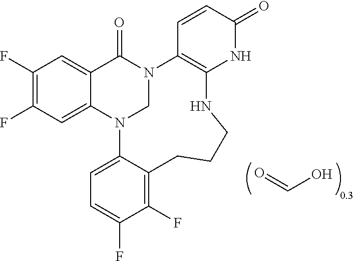

Following the procedure outlined in Example 27, Step F, stirring the reaction mixture at 80° C. for 3 days, 2,3,8,9-tetrafluoro-10,11,12,13-tetrahydro-19H-5,18-methanodibenzo[f,i]-pyrido[2,3-b][1,4,8]triazacyclotridecine-15,19(14H)-dione, formic acid salt (374.1 mg, 59% yield) was prepared. HPLC/MS 0.92 min (A), [M+H]⁺ 445. ¹H NMR (DMSO-d₆, 400 MHz): δ 10.7-11.8 (m, 1H), 7.84 (dd, 1H, J=8.8, 10.8 Hz), 7.4-7.5 (m, 1H), 7.3-7.3 (m, 2H), 6.35 (dd, 1H, J=6.4, 12.2 Hz), 5.79 (d, 1H, J=8.8 Hz), 5.74 (br dd, 1H, J=4.9, 8.3 Hz), 5.40 (d, 1H, J=11.2 Hz), 4.43 (d, 1H, J=11.2 Hz), 3.4-3.5 (m, 1H), 3.05 (tdd, 1H, J=4.2, 8.7, 13.3 Hz), 2.7-2.8 (m, 2H), 2.0-2.1 (m, 1H), 1.4-1.5 (m, 1H).

### Example 99

### Step A: 2,8,9-Trifluoro-15,19-dioxo-10,11,12,13,14,15-hexahydro-19H-5,18-methano-dibenzo [f,i]pyrido[2,3-b][1,4,8]triazacyclotridecine-3-carbonitrile

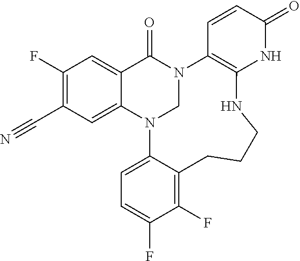 [SMILES: N#Cc1cc2c(cc1F)C(=O)N1CN2c2ccc(F)c(F)c2CCCNc2[nH]c(=O)ccc21]

Following the procedure outlined in Example 55, Step A, with 2,3,8,9-tetrafluoro-10,11,12,13-tetrahydro-19H-5,18-methanodibenzo[f,i]pyrido[2,3-b][1,4,8]triazacyclotridecine-15,19(14H)-dione Example 98 as substrate, stirring at 60° C. for 25 h and quenching with 1N NaOH, 2,8,9-trifluoro-15,19-dioxo-10,11,12,13,14,15-hexahydro-19H-5,18-methanodibenzo[f,i]-pyrido[2,3-b][1,4,8]triazacyclotridecine-3-carbonitrile (30.3 mg, 19% yield) was prepared. HPLC/MS 0.87 min (A), [M+H]⁺ 452. ¹H NMR (DMSO-d₆, 400 MHz): δ 10.3-11.6 (bm, 1H), 7.88 (d, 1H, J=9.3 Hz), 7.4-7.5 (m, 1H), 7.3-7.4 (m, 2H), 6.89 (d, 1H, J=5.4 Hz), 5.7-5.9 (m, 2H), 5.41 (d, 1H, J=11.2 Hz), 4.45 (br d, 1H, J=11.2 Hz), 3.4-3.6 (m, 1H), 3.0-3.1 (m, 1H), 2.7-2.9 (m, 2H), 1.9-2.1 (m, 1H), 1.39 (tdd, 1H, J=4.6, 9.2, 13.8 Hz).

### Example 100

### Step A: 2-Chloro-8-fluoro-15,19-dioxo-10,11,12,13,14,15-hexahydro-19H-5,18-methano-dibenzo[f,i]pyrido[2,3-b][1,4,8]triazacyclotridecine-3-carbonitrile, hydrochloride

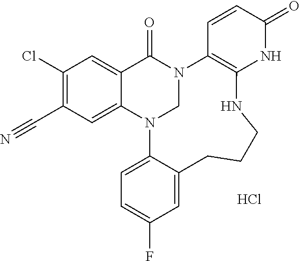 [SMILES: N#Cc1cc2c(cc1Cl)C(=O)N1CN2c2ccc(F)cc2CCCNc2[nH]c(=O)ccc21 ; Cl]

Following the procedure outlined in Example 55, Step A, with 2-chloro-3,8-difluoro-10,11,12,13-tetrahydro-19H-5,18-methanodibenzo[f,i]pyrido[2,3-b][1,4,8]triazacyclotridecine-15,19(14H)-dione Example 96 as substrate and stirring at 80° C. overnight, neutral desired product was prepared, which was taken up in 4M HCl in dioxane and concentrated to afford 2-chloro-8-fluoro-15,19-dioxo-10,11,12,13,14,15-hexahydro-19H-5,18-methanodibenzo[f,i]-pyrido[2,3-b]-[1,4,8]triazacyclotridecine-3-carbonitrile, hydrochloride (23 mg, 33% yield) as a yellow solid. HPLC/MS 0.92 min (A), [M+H]⁺ 450.1. ¹H NMR (DMSO-d₆, 400 MHz): δ 10.72-11.17 (m, 1H) 8.03 (s, 1H) 7.44 (dd, J=8.8, 5.4 Hz, 1H) 7.27-7.36 (m, 2H) 7.21 (td, J=8.4, 3.2 Hz, 1H) 6.78 (s, 1H) 5.69-5.90 (m, 2H) 5.46 (d, J=11.2 Hz, 1H) 4.47 (br d, J=11.2 Hz, 1H) 3.36-3.61 (m, 2H) 2.95 (td, J=8.6, 4.9 Hz, 1H) 2.69-2.81 (m, 1H) 1.90-2.03 (m, 1H) 1.27-1.39 (m, 1H).

### Example 101

### 2-Chloro-3,8,9-trifluoro-10,11,12,13-tetrahydro-19H-5,18-methanodibenzo[f,i]pyrido[2,3-b]-[1,4,8]triazacyclotridecine-15,19(14H)-dione, hydrochloride

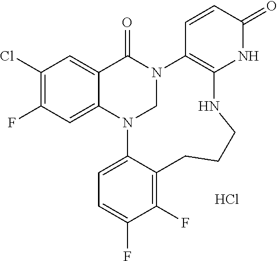 [SMILES: O=C1c2cc(Cl)c(F)cc2N2CN1c1ccc(=O)[nH]c1NCCCc1c2ccc(F)c1F ; Cl]

### Step A: tert-Butyl (3-(2,3-difluoro-6-(2,2,2-trifluoroacetamido)phenyl)prop-2-yn-1-yl)(6-methoxy-3-nitropyridin-2-yl)carbamate 101a

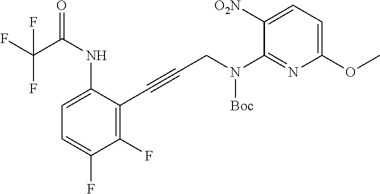 [SMILES: COc1ccc([N+](=O)[O-])c(N(CC#Cc2c(NC(=O)C(F)(F)F)ccc(F)c2F)C(=O)OC(C)(C)C)n1]

Following the procedure outlined in Example 96, Step A, substituting Int-3e with Int-3m and stirring at rt for 4 h, tert-butyl (3-(2,3-difluoro-6-(2,2,2-trifluoroacetamido)phenyl)prop-2-yn-1-yl)(6-methoxy-3-nitropyridin-2-yl)carbamate 101a (2.42 g, 64% yield) was prepared as a yellow powder. HPLC/MS 1.31 min (B), [M+H]⁺ 531.1. ¹H NMR (CDCl₃, 400 MHz): δ 8.64 (br s, 1H) 8.22 (d, J=8.8 Hz, 1H) 8.09 (ddd, J=9.4, 4.0, 2.2 Hz, 1H) 7.13-7.23 (m, 1H) 6.68 (d, J=8.8 Hz, 1H) 5.02 (br s, 2H) 4.03 (s, 3H) 1.41 (br s, 9H).

### Step B: tert-Butyl (3-amino-6-methoxypyridin-2-yl)(3-(2,3-difluoro-6-(2,2,2-trifluoro-acetamido)phenyl)propyl)carbamate 101b

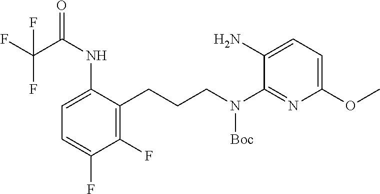 [SMILES: COc1ccc(N)c(N(CCCc2c(NC(=O)C(F)(F)F)ccc(F)c2F)C(=O)OC(C)(C)C)n1]

Following the procedure outlined in Example 96, Step B, stirring at 25° C. overnight, tert-butyl (3-amino-6-methoxypyridin-2-yl)(3-(2,3-difluoro-6-(2,2,2-trifluoroacetamido)phenyl)-propyl)carbamate 101b (2.34 g, 96% yield) was prepared as a yellow solid. HPLC/MS 1.15 min (B), [M+H]⁺ 505.1. ¹H NMR (CDCl₃, 400 MHz): δ 7.42-7.47 (m, 1H) 7.17-7.29 (m, 2H) 6.70 (d, J=8.8 Hz, 1H) 3.98 (s, 3H) 3.73-3.90 (m, 2H) 2.87 (br s, 2H) 1.94-2.03 (m, 2H) 1.60-1.75 (m, 9H).

### Step C: tert-Butyl (3-(2-bromo-5-chloro-4-fluorobenzamido)-6-methoxypyridin-2-yl)(3-(2,3-difluoro-6-(2,2,2-trifluoroacetamido)phenyl)propyl)carbamate 101c

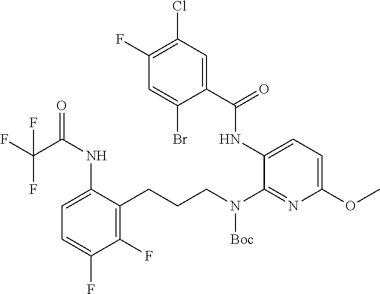 [SMILES: COc1ccc(NC(=O)c2cc(Cl)c(F)cc2Br)c(N(CCCc2c(NC(=O)C(F)(F)F)ccc(F)c2F)C(=O)OC(C)(C)C)n1]

Following the procedure outlined in Example 96, Step C, stirring overnight, tert-butyl (3-(2-bromo-5-chloro-4-fluorobenzamido)-6-methoxypyridin-2-yl)(3-(2,3-difluoro-6-(2,2,2-trifluoroacetamido)phenyl)propyl)carbamate 101c (2.63 g, 74% yield) was prepared as a white solid. HPLC/MS 1.49 min (B), [M+H]⁺ 739.1, 741.0. ¹H NMR (CDCl₃, 400 MHz): δ 8.39-8.56 (m, 1H) 8.17-8.32 (m, 1H) 7.83-8.01 (m, 1H) 7.56 (br d, J=6.8 Hz, 1H) 7.44 (d, J=8.3 Hz, 1H) 7.18-7.25 (m, 1H) 7.07 (q, J=8.8 Hz, 1H) 6.74 (d, J=8.3 Hz, 1H) 3.87 (s, 5H) 2.60 (br t, J=7.1 Hz, 2H) 1.83-1.97 (m, 2H) 1.43 (s, 9H).

### Step D: tert-Butyl (3-(6-amino-2,3-difluorophenyl)propyl)(3-(2-bromo-5-chloro-4-fluorobenzamido)-6-methoxypyridin-2-yl)carbamate 101d

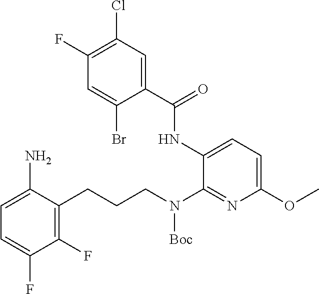 [SMILES: COc1ccc(NC(=O)c2cc(Cl)c(F)cc2Br)c(N(CCCc2c(N)ccc(F)c2F)C(=O)OC(C)(C)C)n1]

Following the procedure outlined in Example 96, Step D, stirring at RT overnight, tert-butyl (3-(6-amino-2,3-difluorophenyl)propyl)(3-(2-bromo-5-chloro-4-fluorobenzamido)-6-methoxy-pyridin-2-yl)carbamate 101d (1.31 g, 57% yield) was prepared as a white solid. HPLC/MS 1.49 min (B), [M+H]⁺ 641.0. ¹H NMR (CDCl₃, 400 MHz): δ 8.63 (br s, 1H) 8.17 (br s, 1H) 7.77 (br s, 1H) 7.39 (br d, J=7.3 Hz, 1H) 7.27 (d, J=8.3 Hz, 1H) 6.98 (br dd, J=8.6, 3.7 Hz, 1H) 6.87 (q, J=9.0 Hz, 1H) 6.55 (d, J=8.3 Hz, 1H) 3.63-3.75 (m, 5H) 2.42 (br t, J=7.3 Hz, 2H) 1.77 (quin, J=7.3 Hz, 2H) 1.27 (s, 9H).

### Step E: tert-Butyl 2-chloro-3,8,9-trifluoro-15-methoxy-19-oxo-5,10,11,12,18,19-hexahydro-13H-dibenzo[f,i]pyrido[2,3-b][1,4,8]triazacyclotridecine-13-carboxylate 101e

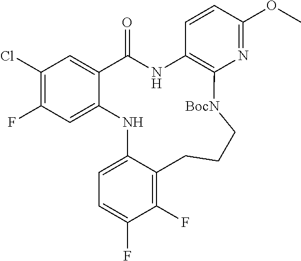 [SMILES: COc1ccc2c(n1)N(C(=O)OC(C)(C)C)CCCc1c(ccc(F)c1F)Nc1cc(F)c(Cl)cc1C(=O)N2]

Following the procedure outlined in Example 96, Step E, heating at 95° C. overnight, tert-butyl 2-chloro-3,8,9-trifluoro-15-methoxy-19-oxo-5,10,11,12,18,19-hexahydro-13H-dibenzo-[f,i]pyrido[2,3-b][1,4,8]triazacyclotridecine-13-carboxylate 101e (510 mg, 33% yield) was prepared as a white solid, which was used without further purification. HPLC/MS 1.47 min (B), [M+H]⁺ low ionization.

### Step F: tert-Butyl 2-chloro-3,8,9-trifluoro-15-methoxy-19-oxo-11,12-dihydro-19H-5,18-methanodibenzo[f,i]pyrido[2,3-b][1,4,8]triazacyclotridecine-13(10H)-carboxylate 101f

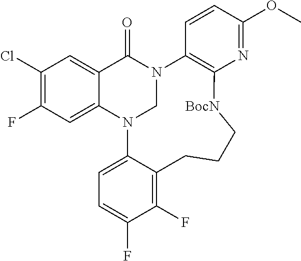 [SMILES: COc1ccc2c(n1)N(C(=O)OC(C)(C)C)CCCc1c(ccc(F)c1F)N1CN2C(=O)c2cc(Cl)c(F)cc21]

Following the procedure outlined in Example 27, Step E, stirring the reaction mixture at 90° C. for 5 days, tert-butyl 2-chloro-3,8,9-trifluoro-15-methoxy-19-oxo-11,12-dihydro-19H-5,18-methanodibenzo[f,i]pyrido[2,3-b][1,4,8]triazacyclotridecine-13(10H)-carboxylate 101f (89 mg, 20% yield) was prepared as a light tan solid. HPLC/MS 1.52 min (B), [M+H]⁺ 575.2. ¹H NMR (CDCl₃, 400 MHz): δ 8.16 (d, J=8.3 Hz, 1H) 7.68 (br s, 1H) 7.03-7.14 (m, 2H) 6.71 (d, J=8.3 Hz, 1H) 6.27-6.43 (m, 1H) 5.15 (br d, J=8.8 Hz, 1H) 4.22 (br s, 2H) 3.87-3.95 (m, 4H) 3.32 (br d, J=7.8 Hz, 1H) 2.85-3.00 (m, 1H) 2.11 (br s, 1H) 1.18 (s, 9H).

### Step G: 2-Chloro-3,8,9-trifluoro-10,11,12,13-tetrahydro-19H-5,18-methanodibenzo-[f,i]pyrido[2,3-b][1,4,8]triazacyclotridecine-15,19(14H)-dione, hydrochloride

### Example 101

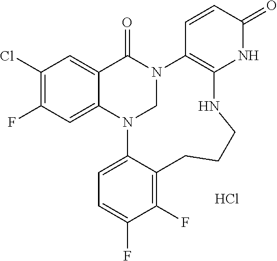 [SMILES: O=C1c2cc(Cl)c(F)cc2N2CN1c1ccc(=O)[nH]c1NCCCc1c2ccc(F)c1F ; Cl]

Following the procedure outlined in Example 27, Step F, stirring the reaction mixture at 90° C. for 2 days, 2-chloro-3,8,9-trifluoro-10,11,12,13-tetrahydro-19H-5,18-methanodibenzo[f,i]-pyrido[2,3-b][1,4,8]triazacyclotridecine-15,19(14H)-dione, hydrochloride (18.7 mg, 27% yield) was prepared as a white solid. HPLC/MS 1.04 min (B), [M+H]⁺ 461.1. ¹H NMR (DMSO-d₆, 400 MHz): δ 10.63-11.01 (m, 1H) 7.96 (d, J=8.3 Hz, 1H) 7.37-7.53 (m, 1H) 7.28-7.34 (m, 2H) 6.32 (d, J=11.2 Hz, 1H) 5.71-5.82 (m, 2H) 5.40 (d, J=11.2 Hz, 1H) 4.43 (d, J=10.8 Hz, 1H) 3.38-3.55 (m, 1H) 3.01 (td, J=9.0, 4.4 Hz, 1H) 2.64-2.84 (m, 2H) 2.00 (br dd, J=8.6, 4.2 Hz, 1H) 1.38 (td, J=8.8, 4.4 Hz, 1H).

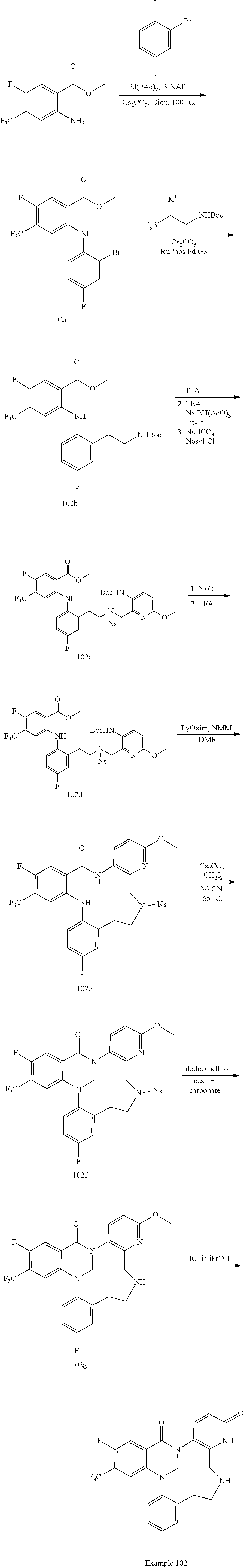

### Example 102

### 2,8-Difluoro-3-(trifluoromethyl)-10,11,12,13-tetrahydro-19H-5,18-methanodibenzo[b,l]pyrido[3,2-f][1,5,9]triazacyclotridecine-15,19(14H)-dione

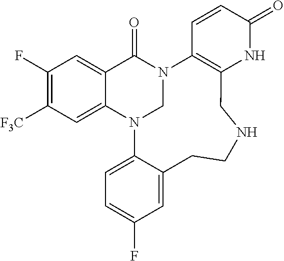 [SMILES: O=C1c2cc(F)c(C(F)(F)F)cc2N2CN1c1ccc(=O)[nH]c1CNCCc1cc(F)ccc12]

### Step A: Methyl 2-((2-bromo-4-fluorophenyl amino)-5-fluoro-4-(trifluoromethyl benzoate 102a

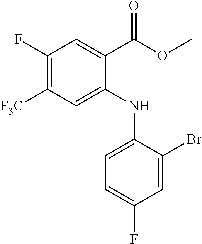 [SMILES: COC(=O)c1cc(F)c(C(F)(F)F)cc1Nc1ccc(F)cc1Br]

Following the procedure outlined in Example 44, Step A, substituting 2-bromo-5-fluorobenzaldehyde with 1-fluoro-3-bromo-4-iodobenzene and heating to 100° C. overnight, methyl 2-((2-bromo-4-fluorophenyl)amino)-5-fluoro-4-(trifluoromethyl)benzoate 102a (5.00 g, 73% yield) was prepared as a colorless solid. HPLC/MS 1.51 min (G), [M+H]⁺ low ionization. ¹H NMR (CDCl₃, 400 MHz): δ 9.33 (s, 1H), 7.82 (d, J=10.8 Hz, 1H), 7.45 (dd, J=7.8, 2.9 Hz, 1H), 7.37 (dd, J=9.0, 5.1 Hz, 1H), 7.14 (d, J=5.9 Hz, 1H), 7.07-7.12 (m, 1H), 3.99 (s, 3H).

### Step B: Methyl 2-((2-(2-((tert-butoxycarbonyl)amino)ethyl)-4-fluorophenyl)amino)-5-fluoro-4-(trifluoromethyl)benzoate 102b

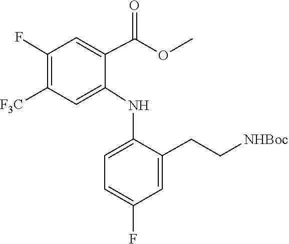 [SMILES: COC(=O)c1cc(F)c(C(F)(F)F)cc1Nc1ccc(F)cc1CCNC(=O)OC(C)(C)C]

Methyl 2-((2-bromo-4-fluorophenyl)amino)-5-fluoro-4-(trifluoromethyl)benzoate 102a (5.00 g, 12.2 mmol), potassium 2-(tert-butoxycarbonylamino)-ethyltrifluoroborate (4.59 g, 18.3 mmol), Cs₂CO₃ (11.9 g, 36.6 mmol), and (2-dicyclohexylphosphino-2′,6′-diisopropoxy-1,1′-biphenyl)[2-(2′-amino-1,1′-biphenyl)]palladium(II) methanesulfonate (509.8 mg, 609.5 μmol) were purged with N₂ for 15 minutes followed by the addition of degassed toluene (81.3 mL) and H₂O (20.3 mL). The reaction mixture was heated to 100° C. overnight, then cooled to ambient temperature, passed through a column of Celite and washed with EtOAc. The resulting organic phase was washed with water and brine, passed through a plug of MgSO₄, concentrated and purified by silica gel flash column chromatography to afford methyl 2-((2-(2-((tert-butoxycarbonyl)amino)ethyl)-4-fluorophenyl)amino)-5-fluoro-4-(trifluoromethyl)benzoate (4.70 g, 81%) as a colorless solid. HPLC/MS 1.49 min (G), [M+H]⁺ 475.2. ¹H NMR NMR (DMSO-d₆, 400 MHz): δ 9.00 (s, 1H), 7.85 (d, J=11.2 Hz, 1H), 7.32 (dd, J=8.6, 5.6 Hz, 1H), 7.09-7.21 (m, 2H), 6.86 (d, J=5.9 Hz, 1H), 6.81 (br t, J=5.6 Hz, 1H), 3.92 (s, 3H), 3.09-3.18 (m, 2H), 2.66 (t, J=6.8 Hz, 2H), 1.28 (s, 9H).

### Step C: Methyl 2-((2-(2-((N-((3-((tert-butoxycarbonyl)amino)-6-methoxypyridin-2-yl)-methyl)-4-nitrophenyl)sulfonamido)ethyl)-4-fluorophenyl)amino)-5-fluoro-4-(trifluoro-methyl)benzoate 102c

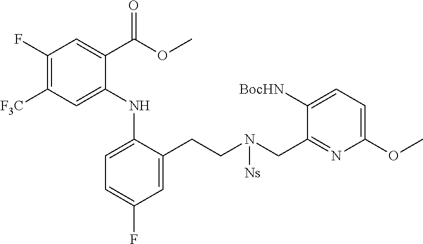

Methyl 2-((2-(2-((tert-butoxycarbonyl)amino)ethyl)-4-fluorophenyl)amino)-5-fluoro-4-(trifluoromethyl)benzoate (4.70 g, 9.91 mmol) was added to a 200 mL round bottomed flask equipped with a magnetic stirring bar. DCM (49.5 mL) was added, followed by 2,2,2-trifluoroacetic acid (5.65 g, 49.5 mmol) and the reaction mixture was stirred for 96 h. The mixture was concentrated in-vacuo to afford an oil that that was taken up in EtOAc and H₂O. The aqueous layer was made basic with aq. NaOH, the layers separated, and the organic layer was washed with brine. 4N HCl in dioxane was added to the resulting organic layer and the precipitate filtered and washed with Et₂O to afford a colorless solid as the hydrochloride salt. The hydrochloride salt and tert-butyl (2-formyl-6-methoxypyridin-3-yl)carbamate (2.70 g, 10.7 mmol) were added to a 250 mL round bottomed flask equipped with a magnetic stirring bar. Trifluorotoluene (48.7 mL) was added and the reaction mixture was stirred for 15 min prior to the addition of TEA (1.02 mL, 7.30 mmol) and sodium triacetoxyborohydride (4.54 g, 21.4 mmol). The reaction mixture was sealed and stirred for 30 min, to which was added a 1M sol'n of NaHCO₃ (146 mL, 146.4 mmol), followed by of trifluorotoluene (10 mL) and the reaction mixture was stirred until fully quenched. 4-Nitrobenzene-1-sulfonylchloride (3.24 g, 14.6 mmol) was added and the reaction mixture was stirred at RT overnight. The reaction mixture was diluted with EtOAc and H₂O, the layers separated, the organic phase washed with H₂O, brine, passed through MgSO₄, and the solvent concentrated under reduced pressure. The crude residue was dissolved in DCM and purified by silica gel flash column chromatography. Product fractions were combined and evaporated under reduced pressure to afford methyl 2-((2-(2-((N-((3-((tert-butoxycarbonyl)amino)-6-methoxypyridin-2-yl)methyl)-4-nitrophenyl)sulfonamido)ethyl)-4-fluorophenyl)amino)-5-fluoro-4-(trifluoromethyl)benzoate 102c (2.80 g, 36% yield). HPLC/MS 1.62 min (A), [M-^{t}Bu]⁺ 740.4. ¹H NMR NMR (CD₂Cl₂, 400 MHz): δ 9.06 (s, 1H), 8.20-8.26 (m, 2H), 7.95 (br dd, J=8.6, 4.6 Hz, 1H), 7.84 (d, J=10.8 Hz, 1H), 7.80 (d, J=8.8 Hz, 2H), 7.20 (dd, J=8.6, 5.1 Hz, 1H), 7.03 (td, J=8.3, 2.9 Hz, 2H), 6.91 (dd, J=9.3, 2.9 Hz, 1H), 6.74 (d, J=5.9 Hz, 1H), 6.68 (d, J=8.8 Hz, 1H), 4.43 (s, 2H), 3.98 (s, 3H), 3.61 (s, 3H), 3.49-3.56 (m, 2H), 2.70-2.80 (m, 2H), 1.55 (s, 9H).

### Step D: 2-((2-(2-((N-((3-Amino-6-methoxypyridin-2-yl)methyl)-4-nitrophenyl)-sulfonamido)ethyl)-4-fluorophenyl)amino)-5-fluoro-4-(trifluoromethyl)benzoic 102d

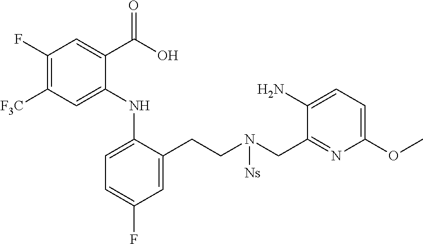

Methyl 2-((2-(2-((N-((3-((tert-butoxycarbonyl)amino)-6-methoxypyridin-2-yl)methyl)-4-nitrophenyl)sulfonamido)ethyl)-4-fluorophenyl)amino)-5-fluoro-4-(trifluoromethyl)benzoate 102c (2.80 g, 3.52 mmol), DCM (12.0 mL), and TFA (2.71 mL, 35.2 mmol) were heated at 50° C. overnight and concentrated under reduced pressure. THF (12.0 mL) was added, followed by a 5M aq. sol'n of NaOH (10.6 mL, 52.8 mmol), and the reaction mixture was heated for 4 hours at 50° C. then cooled to ambient temperature. The mixture was acidified with 1N HCl, diluted with EtOAc, the layers separated and the organic phase was washed with brine, passed through a plug of MgSO₄, filtered and concentrated under reduced pressure to afford 2-((2-(2-((N-((3-amino-6-methoxypyridin-2-yl)methyl)-4-nitrophenyl)sulfonamido)ethyl)-4-fluorophenyl)-amino)-5-fluoro-4-(trifluoromethyl)benzoic acid 102d, which was used without further purification. HPLC/MS 1.44 min (G), [M-^{t}Bu]⁺ 682.1.

### Step E: 2,8-Difluoro-15-methoxy-12-((4-nitrophenyl)sulfonyl)-3-(trifluoromethyl)-5,10,11,12,13,18-hexahydro-19H-dibenzo[b,l]pyrido[3,2-f][1,5,9]triazacyclotridecin-19-one

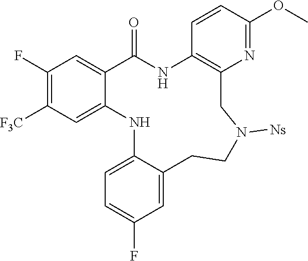

Following the procedure outlined in Example 44, Step E, stirring the reaction mixture at RT overnight, 2,8-difluoro-15-methoxy-12-((4-nitrophenyl)sulfonyl)-3-(trifluoromethyl)-5,10,11,12,13,18-hexahydro-19H-dibenzo[b,l]pyrido[3,2-f][1,5,9]triazacyclo-tridecin-19-one 102e (1.70 g, 67% yield) was prepared. HPLC/MS 1.38 min (G), [M+H]⁺ 664.1 ¹H NMR (DMSO-d₆, 400 MHz): δ 9.95 (s, 1H), 8.40 (d, J=9.3 Hz, 2H), 8.19 (d, J=8.8 Hz, 2H), 7.96 (d, J=8.8 Hz, 1H), 7.62-7.76 (m, 2H), 7.36 (d, J=6.4 Hz, 1H), 6.87-7.01 (m, 2H), 6.83 (dd, J=8.6, 5.1 Hz, 1H), 6.73 (d, J=8.8 Hz, 1H), 4.16 (s, 2H), 3.64 (s, 3H), 3.28-3.37 (m, 2H), 3.00 (br t, J=5.9 Hz, 2H).

### Step F: 2,8-Difluoro-15-methoxy-12-((4-nitrophenyl)sulfonyl)-3-(trifluoromethyl)-10,11,12,13-tetrahydro-19H-5,18-methanodibenzo[b,l]pyrido[3,2-f][1,5,9]triazacyclotridecin-19-one 102f

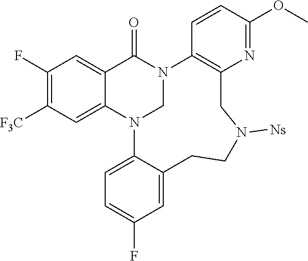

Following the procedure outlined in Example 27, Step E, stirring the reaction mixture at 65° C. for 48 h, 2,8-difluoro-15-methoxy-12-((4-nitrophenyl)sulfonyl)-3-(trifluoromethyl)-10,11,12,13-tetrahydro-19H-5,18-methanodibenzo[b,l]pyrido[3,2-f][1,5,9]triazacyclotridecin-19-one 102f was prepared as an off-white solid. HPLC/MS 1.38 min (G), [M+H]⁺ 676.1. ¹H NMR (CDCl₃, 400 MHz): δ 8.34-8.42 (m, 2H), 8.26-8.32 (m, 2H), 7.94 (d, J=10.3 Hz, 1H), 7.52 (br d, J=8.8 Hz, 1H), 7.32 (dd, J=8.6, 5.1 Hz, 1H), 7.24 (dd, J=8.8, 2.9 Hz, 1H), 7.15 (td, J=8.1, 2.9 Hz, 1H), 6.84 (d, J=8.8 Hz, 1H), 6.54 (d, J=5.4 Hz, 1H), 5.58 (d, J=11.2 Hz, 1H), 4.79-4.94 (m, 1H), 4.45 (br d, J=13.7 Hz, 1H), 3.89 (br s, 3H), 3.18-3.31 (m, 2H), 2.64-2.78 (m, 1H), 1.58 (br s, 2H).

### Step G: 2,8-Difluoro-15-methoxy-3-(trifluoromethyl)-10,11,12,13-tetrahydro-19H-5,18-methanodibenzo[b,l]pyrido[3,2-f][1,5,9]triazacyclotridecin-19-one 1022

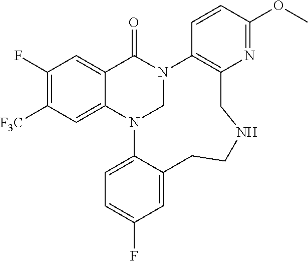 [SMILES: COc1ccc2c(n1)CNCCc1cc(F)ccc1N1CN2C(=O)c2cc(F)c(C(F)(F)F)cc21]

2,8-Difluoro-15-methoxy-12-((4-nitrophenyl)sulfonyl)-3-(trifluoromethyl)-10,11,12,13-tetrahydro-19H-5,18-methanodibenzo[b,l]pyrido[3,2-f][1,5,9]triazacyclotridecin-19-one 102f (500.0 mg, 740 μmol), Cs₂CO₃ (723.4 mg, 2.22 mmol) acetonitrile (2.96 mL) and dodecanthiol (449.4 mg, 2.22 mmol) were stirred at 50° C. overnight, concentrated and partitioned between H₂O and EtOAc. The organic phase was passed through a plug of MgSO₄, filtered, concentrated under reduced pressure, then purified by silica gel flash column chromatography to afford 2,8-difluoro-15-methoxy-3-(trifluoromethyl)-10,11,12,13-tetrahydro-19H-5,18-methanodibenzo[b,l]pyrido-[3,2-f][1,5,9]triazacyclotridecin-19-one 102g (210.0 mg, 58%) as an off-white solid, that was used in the next step without further purification. HPLC/MS 0.75 min (G), [M+H]⁺ 491.2.

### Step H: 2,8-Difluoro-3-(trifluoromethyl)-10,11,12,13-tetrahydro-19H-5,18-methano-dibenzo[b,l]pyrido[3,2-f][1,5,9]triazacyclotridecine-15,19(14H)-dione 102h

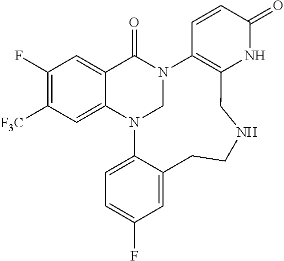 [SMILES: O=C1c2cc(F)c(C(F)(F)F)cc2N2CN1c1ccc(=O)[nH]c1CNCCc1cc(F)ccc12]

2,8-Difluoro-15-methoxy-3-(trifluoromethyl)-10,11,12,13-tetrahydro-19H-5,18-methano-dibenzo[b,l]pyrido[3,2-f][1,5,9]triazacyclotridecin-19-one 102g (210.0 mg, 428.2 μmol) and 5M HCl in isopropanol (4.28 mL, 21.4 mmol) were heated to 90° C. overnight. The reaction mixture was cooled to ambient temperature, heptanes were added, and the mixture was stirred for 5 min, filtered and washed with heptanes and dried under vacuo to afford 2,8-difluoro-3-(trifluoromethyl)-10,11,12,13-tetrahydro-19H-5,18-methano-dibenzo[b,l]pyrido[3,2-f][1,5,9]-triazacyclotridecine-15,19(14H)-dione (70.0 mg, 34%). HPLC/MS 0.79 min (A), [M+H]⁺ 477.1. ¹H NMR (DMSO-d₆, 400 MHz): δ 10.37-10.08 (m, 1H), 9.10-8.86 (m, 1H), 7.94 (br dd, J=5.1, 2.2 Hz, 1H), 7.69-7.45 (m, 3H), 7.31 (td, J=8.4, 3.2 Hz, 1H), 6.66 (d, J=9.3 Hz, 1H), 6.54 (d, J=5.9 Hz, 1H), 5.60-5.46 (m, 1H), 5.08 (d, J=11.7 Hz, 1H), 4.09-3.83 (m, 2H), 3.33-3.14 (m, 1H), 3.11-2.95 (m, 1H), 2.90-2.66 (m, 2H).

### Example 103

### 8-Fluoro-2-(trifluoromethyl)-10,11,12,13-tetrahydro-19H-5,18-methanodibenzo[b,l]pyrido[3,2-f][1,5,9]triazacyclotridecine-15,19(14H)-dione

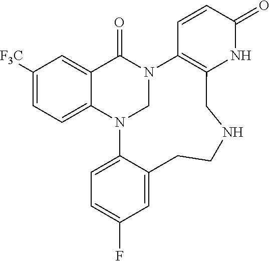 [SMILES: O=C1c2cc(C(F)(F)F)ccc2N2CN1c1ccc(=O)[nH]c1CNCCc1cc(F)ccc12]

### Step A: Methyl 2-((2-(2-((tert-butoxycarbonyl)amino)ethyl)-4-fluorophenyl)amino)-5-(trifluoromethyl)benzoate 103a

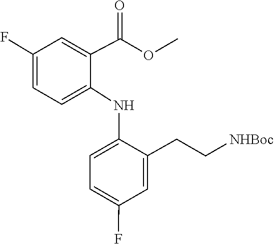 [SMILES: COC(=O)c1cc(F)ccc1Nc1ccc(F)cc1CCNC(=O)OC(C)(C)C]

Following the procedure outlined in Example 102, Step B, using methyl 2-((2-bromo-4-fluorophenyl)amino)-5-(trifluoromethyl)benzoate Int-1f as substrate, and stirring at 80° C. for 16 h, methyl 2-((2-(2-((tert-butoxycarbonyl)amino)ethyl)-4-fluorophenyl)amino)-5-(trifluoro-methyl)benzoate 103a (3.70 g, 79% yield) was prepared as a colorless solid. HPLC/MS 1.47 min (G), [M+H]⁺ 457.2. ¹H NMR (CDCl₃, 400 MHz): δ 9.52 (s, 1H), 8.26 (d, J=1.5 Hz, 1H), 7.43-7.49 (m, 1H), 7.25 (dd, J=8.3, 5.4 Hz, 1H), 7.08 (dd, J=9.5, 2.7 Hz, 1H), 6.98-7.05 (m, 1H), 6.68 (d, J=8.8 Hz, 1H), 4.51-4.63 (m, 1H), 3.98 (s, 3H), 3.31-3.42 (m, 2H), 2.78 (t, J=7.1 Hz, 2H), 1.41 (s, 9H).

### Step B: Methyl 2-((2-(2-aminoethyl)-4-fluorophenyl)amino)-5-(trifluoromethyl)benzoate hydrochloride 103b

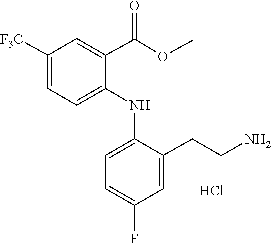 [SMILES: COC(=O)c1cc(C(F)(F)F)ccc1Nc1ccc(F)cc1CCN ; Cl]

Following the Boc-deprotection procedure outlined in Int-1h, Step B, methyl 2-((2-(2-aminoethyl)-4-fluorophenyl)amino)-5-(trifluoromethyl)benzoate, hydrochloride 103b (2.66 g, 88% yield) was prepared as a colorless solid. HPLC/MS 0.76 min (G), [M+H]⁺ 357.2. ¹H NMR (DMSO-d₆, 400 MHz): δ 9.37 (s, 1H), 8.14 (d, J=2.0 Hz, 1H), 7.99 (br s, 3H), 7.63 (dd, J=8.8, 2.4 Hz, 1H), 7.31-7.38 (m, 2H), 7.20-7.27 (m, 1H), 6.56 (d, J=8.8 Hz, 1H), 3.92 (s, 3H), 2.79-2.87 (m, 2H), 2.51 (dt, J=3.5, 1.9 Hz, 2H).

### Step C: Methyl 2-((2-(2-((N-((3-((tert-butoxycarbonyl)amino)-6-methoxypyridin-2-yl)-methyl)-4-nitrophenyl)sulfonamido)ethyl)-4-fluorophenyl)amino)-5-(trifluoromethyl)benzoate 103c

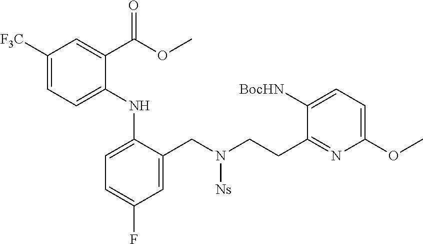

Following the procedure outlined in Example 94, Step C, methyl 2-((2-(2-((N-((3-((tert-butoxycarbonyl)amino)-6-methoxypyridin-2-yl)methyl)-4-nitrophenyl)sulfonamido)ethyl)-4-fluorophenyl)amino)-5-(trifluoromethyl)benzoate 103c (410 mg, 39% yield) was prepared. HPLC/MS 1.62 min (G), [M+H]⁺ 778.2. ¹H NMR (CDCl₃, 400 MHz): δ 9.43 (s, 1H), 8.27 (d, J=1.5 Hz, 1H), 8.21 (d, J=8.8 Hz, 2H), 7.99 (br d, J=7.8 Hz, 1H), 7.75 (d, J=8.8 Hz, 2H), 7.44 (dd, J=8.8, 2.4 Hz, 1H), 7.18 (dd, J=8.6, 5.1 Hz, 1H), 7.07 (br d, J=2.0 Hz, 1H), 7.00 (td, J=7.9, 2.7 Hz, 1H), 6.85 (dd, J=8.8, 2.9 Hz, 1H), 6.69 (d, J=8.8 Hz, 1H), 6.55 (d, J=8.8 Hz, 1H), 4.39 (s, 2H), 3.97 (s, 3H), 3.62 (s, 3H), 3.41-3.51 (m, 2H), 2.67-2.76 (m, 2H), 1.55 (s, 9H).

### Step D: 2-((2-(2-((N-((3-Amino-6-methoxypyridin-2-yl)methyl)-4-nitrophenyl)-sulfonamido)ethyl)-4-fluorophenyl)amino)-5-fluoro-4-(trifluoromethyl)benzoic 103d

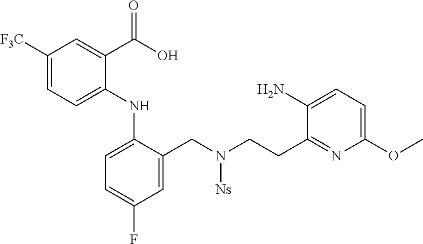

Following the procedure outlined in Example 102, Step D, 2-((2-(2-((N-((3-amino-6-methoxypyridin-2-yl)methyl)-4-nitrophenyl)sulfonamido)ethyl)-4-fluorophenyl)amino)-5-(trifluoromethyl)benzoic acid 103d (160 mg, 46% yield) was prepared, and used as is in the next step. HPLC/MS 1.42 min (G), [M+H]⁺ 664.2.

### Step E: 8-Fluoro-15-methoxy-12-((4-nitrophenyl)sulfonyl)-2-(trifluoromethyl)-5,10,11,12,13,18-hexahydro-19H-dibenzo[b,l]pyrido[3,2-f][1,5,9]triazacyclotridecin-19-one 103e

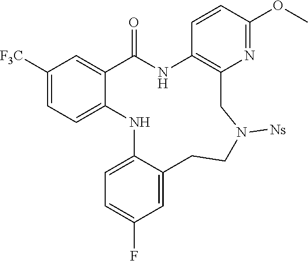

Following the procedure outlined in Example 44, Step E, stirring the reaction mixture at room temperature overnight, 8-fluoro-15-methoxy-12-((4-nitrophenyl)sulfonyl)-2-(trifluoro-methyl)-5,10,11,12,13,18-hexahydro-19H-dibenzo[b,l]pyrido[3,2-f][1,5,9]triazacyclo-tridecin-19-one 103e (105 mg, 68% yield) was prepared. HPLC/MS 1.38 min (G), [M+H]⁺ 646.2. ¹H NMR (DMSO-d₆, 400 MHz): δ 9.78 (s, 1H), 8.42 (br d, J=8.8 Hz, 2H), 8.20 (br d, J=8.8 Hz, 2H), 8.14 (br d, J=8.8 Hz, 1H), 8.00 (s, 1H), 7.78 (s, 1H), 7.69 (br d, J=8.3 Hz, 1H), 6.98-7.09 (m, 3H), 6.89-6.96 (m, 1H), 6.74 (d, J=8.8 Hz, 1H), 4.27 (br s, 2H), 3.68 (s, 3H), 3.30 (br s, 2H), 3.00 (br d, J=5.4 Hz, 2H).

### Step F: 8-Fluoro-15-methoxy-12-((4-nitrophenyl)sulfonyl)-2-(trifluoromethyl)-10,11,12,13-tetrahydro-19H-5,18-methanodibenzo[b,l]pyrido[3,2-f][1,5,9]triazacyclotridecin-19-one 103f

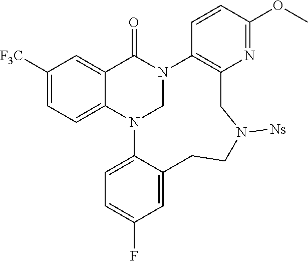

Following the procedure outlined in Example 27, Step E, stirring the reaction mixture at 85° C. overnight, 8-fluoro-15-methoxy-12-((4-nitrophenyl)sulfonyl)-2-(trifluoromethyl)-10,11,12,13-tetrahydro-19H-5,18-methanodibenzo[b,l]pyrido[3,2-f][1,5,9]triazacyclotridecin-19-one 103f (450 mg, 59% yield) was prepared as an off white solid. HPLC/MS 1.38 min (G), [M+H]⁺ 658.2.

### Step G: 8-Fluoro-15-methoxy-2-(trifluoromethyl)-10,11,12,13-tetrahydro-19H-5,18-methanodibenzo[b,l]pyrido[3,2-f][1,5,9]triazacyclotridecin-19-one 1032

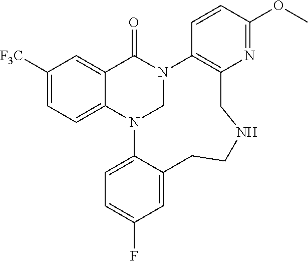 [SMILES: COc1ccc2c(n1)CNCCc1cc(F)ccc1N1CN2C(=O)c2cc(C(F)(F)F)ccc21]

Following the procedure outlined in Example 102, Step G, 8-fluoro-15-methoxy-2-(trifluoromethyl)-10,11,12,13-tetrahydro-19H-5,18-methanodibenzo[b,l]pyrido[3,2-f][1,5,9]-triazacyclotridecin-19-one 103g (290 mg, 100% yield) was prepared as an off white solid. HPLC/MS 0.74 min (G), [M+H]⁺ 473.1. ¹H NMR (DMSO-d₆, 400 MHz): δ 8.10 (d, J=2.0 Hz, 1H), 7.75 (d, J=8.3 Hz, 1H), 7.65 (dd, J=9.0, 2.2 Hz, 1H), 7.38-7.45 (m, 2H), 7.21 (td, J=8.6, 2.9 Hz, 1H), 6.84 (d, J=8.3 Hz, 1H), 6.34 (d, J=8.8 Hz, 1H), 5.45 (d, J=9.8 Hz, 1H), 5.21 (d, J=9.3 Hz, 1H), 3.99-4.08 (m, 1H), 3.83-3.93 (m, 4H), 3.37 (d, J=10.3 Hz, 1H), 3.07-3.12 (m, 1H), 2.93-2.99 (m, 1H), 2.43 (br d, J=13.2 Hz, 1H).

### Step H: 8-Fluoro-2-(trifluoromethyl)-10,11,12,13-tetrahydro-19H-5,18-methanodibenzo-[b,l]pyrido[3,2-f][1,5,9]triazacyclotridecine-15,19(14H)-dione Example 103

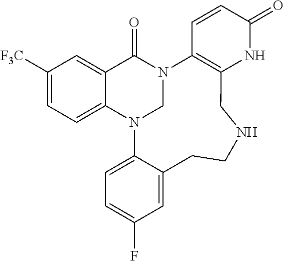 [SMILES: O=C1c2cc(C(F)(F)F)ccc2N2CN1c1ccc(=O)[nH]c1CNCCc1cc(F)ccc12]

Following the procedure outlined in Example 102, Step H, 8-fluoro-2-(trifluoromethyl)-10,11,12,13-tetrahydro-19H-5,18-methanodibenzo[b,l]pyrido[3,2-f][1,5,9]triazacyclotridecine-15,19(14H)-dione, hydrochloride (200 mg, 66%) was prepared as a beige powder. A small fraction of this material was purified on an XSELECT CSH C18 column, eluting with 30-85% AcCN in 10 mM ammonium bicarbonate in H₂O adjusted to pH 10 with ammonia to afford 8-fluoro-2-(trifluoromethyl)-10,11,12,13-tetrahydro-19H-5,18-methanodibenzo[b,l]pyrido[3,2-f][1,5,9]-triazacyclotridecine-15,19(14H)-dione (20 mg, 7% yield). HPLC/MS 0.69 min (G), [M+H]⁺ 459.1. ¹H NMR (CD₃OD, 400 MHz): δ 8.24 (d, J=2.0 Hz, 1H), 7.65 (d, J=9.8 Hz, 1H), 7.61 (dd, J=9.0, 2.2 Hz, 1H), 7.35 (dd, J=8.3, 5.4 Hz, 1H), 7.29 (dd, J=9.3, 2.9 Hz, 1H), 7.14 (td,J=8.3, 2.9 Hz, 1H), 6.54 (d, J=9.8 Hz, 1H), 6.45 (d, J=8.8 Hz, 1H), 5.45 (d, J=9.8 Hz, 1H), 5.21 (d, J=10.3 Hz, 1H), 3.93 (d, J=12.2 Hz, 1H), 3.44 (d, J=12.2 Hz, 1H), 3.10-3.21 (m, 1H), 2.99-3.07 (m, 1H), 2.88-2.97 (m, 1H), 2.45 (dt, J=13.2, 4.2 Hz, 1H).

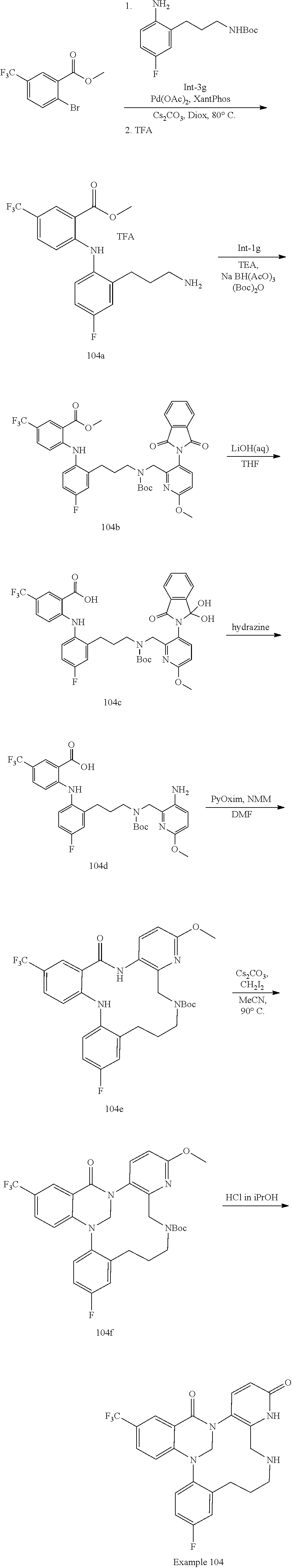

### Example 104

### 3⁴-Fluoro-2⁶-(trifluoromethyl)-1¹,1²,2¹,2²,2³,2⁴-hexahydro-7-aza-2(3,1)quinazolina-1(5,6)-pyridina-3(1,2)-benzenacyclooctaphane-1²,2⁴-dione

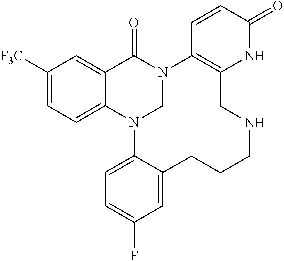 [SMILES: O=C1c2cc(C(F)(F)F)ccc2N2CN1c1ccc(=O)[nH]c1CNCCCc1cc(F)ccc12]

### Step A: Methyl 2-(2-3-((tert-butoxycarbonyl amino)ropy)-4-fluorophenyl amino)-5-(trifluoromethyl)benzoate 104a

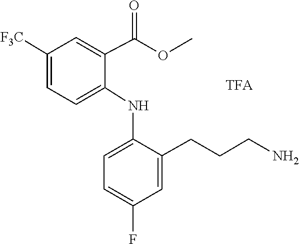 [SMILES: COC(=O)c1cc(C(F)(F)F)ccc1Nc1ccc(F)cc1CCCN]

Following the procedure outlined in Example 3, Step A, substituting 2-bromo-4-fluoroaniline with Int-3g, BINAP with XantPhos, Pd₂(dba)₃ with Pd(OAc)₂, toluene with dioxane and heating to 80° C. for 17 h, methyl 2-((2-(3-((tert-butoxycarbonyl)amino)propyl)-4-fluorophenyl)amino)-5-(trifluoromethyl)benzoate (2.97 g, 90% yield) was prepared as a white solid. HPLC/MS 1.53 min (B), [M+H]⁺ 471. ¹H NMR (CD₃OD, 400 MHz): δ 1.39 (s, 9H) 1.68-1.78 (m, 2H), 2.57-2.65 (m, 2H), 3.01-3.09 (m, 2H), 3.99 (s, 3H), 6.67-6.76 (m, 1H), 7.01-7.09 (m, 1H), 7.12-7.20 (m, 1H), 7.25-7.33 (m, 1H), 7.48-7.56 (m, 1H), 8.22 (d, J=1.47 Hz, 1H), 9.52-9.61 (m, 1H). To methyl 2-((2-(3-((tert-butoxycarbonyl)amino)propyl)-4-fluoro-phenyl)amino)-5-(trifluoromethyl)benzoate (1.35 g, 2.86 mmol) in 1,2-dichloroethane (12 mL) was added TFA (4.41 mL, 57.2 mmol). After 16 h the reaction mixture was concentrated under reduced pressure to afford methyl 2-((2-(3-aminopropyl)-4-fluorophenyl)amino)-5-(trifluoro-methyl)-benzoate, trifluoroacetic acid salt 104a (1.81 g, 100% yield) as a gummy solid. HPLC/MS 0.92 min (A), [M+H]⁺ 371.1. ¹H NMR (CD₃OD, 400 MHz): δ 1.86-1.98 (m, 2H), 2.65-2.75 (m, 2H), 2.87-2.96 (m, 2H), 3.96 (s, 3H), 6.74 (d, J=9.29 Hz, 1H), 7.09 (td, J=8.44, 3.18 Hz, 1H), 7.19 (dd, J=9.29, 2.93 Hz, 1H), 7.33 (dd, J=8.56, 5.14 Hz, 1H), 7.53 (dd, J=9.29, 1.96 Hz, 1H), 8.21 (d, J=1.47 Hz, 1H).

### Step B: Methyl 2-((2-(3-((tert-butoxycarbonyl)((3-(1,3-dioxoisoindolin-2-yl)-6-methoxy-pyridin-2-yl)methyl)amino)propyl)-4-fluorophenyl)amino)-5-(trifluoromethyl)benzoate 104b

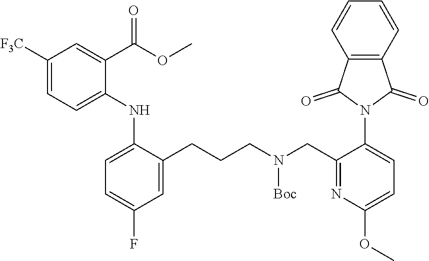 [SMILES: COC(=O)c1cc(C(F)(F)F)ccc1Nc1ccc(F)cc1CCCN(Cc1nc(OC)ccc1N1C(=O)c2ccccc2C1=O)C(=O)OC(C)(C)C]

Following the preparation outlined in Example 44, Step B, substituting methyl 5-fluoro-2-((4-fluoro-2-formylphenyl)amino)-4-(trifluoromethyl)benzoate with 3-(1,3-dioxoisoindolin-2-yl)-6-methoxypicolinaldehyde Int-1g and 2-(6-methoxy-3-nitropyridin-2-yl)ethan-1-amine with 104a, methyl 2-((2-(3-((tert-butoxycarbonyl)((3-(1,3-dioxoisoindolin-2-yl)-6-methoxy-pyridin-2-yl)methyl)amino)propyl)-4-fluorophenyl)amino)-5-(trifluoromethyl)benzoate 104b (1.21 g, 71% yield) was prepared as a white solid. HPLC/MS 1.66 min (B), [M+H]⁺ 737.4. ¹H NMR (CD₃OD, 400 MHz): δ 1.07-1.22 (m, 9H), 1.28-1.36 (m, 2H), 1.57-1.86 (m, 2H), 2.43-2.63 (m, 2H), 3.12-3.22 (m, 1H), 3.84-3.98 (m, 6H), 4.40 (s, 2H), 6.60-6.74 (m, 1H), 6.75-6.87 (m, 1H), 7.03 (br d, J=5.38 Hz, 2H), 7.26 (dd, J=8.80, 5.38 Hz, 1H), 7.43-7.53 (m, 1H), 7.57 (d, J=8.31 Hz, 1H), 7.82-8.03 (m, 4H), 8.11-8.23 (m, 1H), 9.53 (s, 1H).

### Step C: 2-((2-(2-((N-((3-Amino-6-methoxypyridin-2-yl)methyl)-4-nitrophenyl)-sulfonamido)ethyl)-4-fluorophenyl)amino)-5-fluoro-4-(trifluoromethyl)benzoic acid 104c

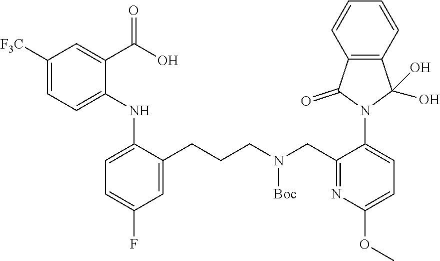 [SMILES: COc1ccc(N2C(=O)c3ccccc3C2(O)O)c(CN(CCCc2cc(F)ccc2Nc2ccc(C(F)(F)F)cc2C(=O)O)C(=O)OC(C)(C)C)n1]

Following the procedure outlined in Example 27, Step B, stirring the reaction mixture at RT overnight, 2-((2-(3-((tert-butoxycarbonyl)((3-(1,1-dihydroxy-3-oxoisoindolin-2-yl)-6-methoxypyridin-2-yl)methyl)amino)propyl)-4-fluorophenyl)amino)-5-(trifluoromethyl)benzoic acid 104c (1.17 g, 99% yield) was prepared as a light brown solid. HPLC/MS 0.90 min (B), [M+H]⁺ 741.4. ¹H NMR (CD₃OD, 400 MHz): δ 1.26 (s, 9H), 1.69-1.99 (m, 2H), 2.47-2.73 (m, 2H), 3.37-3.42 (m, 2H), 3.82 (s, 3H), 4.50 (br s, 2H), 6.62-6.78 (m, 2H), 6.98-7.19 (m, 2H), 7.29 (dd, J=8.80, 5.38 Hz, 1H), 7.48 (dd, J=9.05, 2.20 Hz, 1H), 7.57-7.73 (m, 3H), 8.04 (d, J=7.34 Hz, 2H), 8.21 (d, J=1.47 Hz, 1H).

### Step D: 2-((2-(3-(((3-Amino-6-methoxypyridin-2-yl)methyl)(tert-butoxycarbonyl)-amino)propyl)-4-fluorophenyl)amino)-5-(trifluoromethyl)benzoic acid 104d

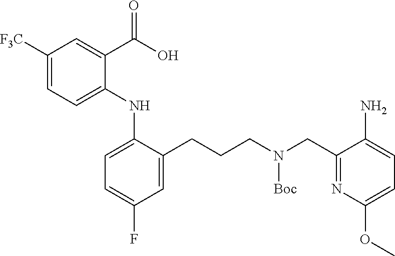 [SMILES: COc1ccc(N)c(CN(CCCc2cc(F)ccc2Nc2ccc(C(F)(F)F)cc2C(=O)O)C(=O)OC(C)(C)C)n1]

To 2-((2-(3-((tert-butoxycarbonyl)((3-(1,1-dihydroxy-3-oxoisoindolin-2-yl)-6-methoxy-pyridin-2-yl)methyl)amino)propyl)-4-fluorophenyl)amino)-5-(trifluoromethyl)benzoic acid 104c (1.16 g, 1.50 mmol) in MeOH (36 mL) was added hydrazine hydrate (37.7 g, 752 mmol) and the reaction mixture was heated at 80° C. overnight. Another portion of hydrazine hydrate (14.6 mL, 200 eq.) was added, and the reaction mixture was heated at 80° C. for another 73 h and concentrated under vacuum. The residue was diluted with EtOAc and washed with water (3×). The organic layer was dried over Na₂SO₄, filtered and concentrated under reduced pressure. The crude residue was purified by silica gel flash column chromatography (80 g), eluting with a 100% DCM to 3% MeOH (3% TEA)-DCM gradient. Pure product fractions were combined and evaporated under reduced pressure to afford 2-((2-(3-(((3-amino-6-methoxypyridin-2-yl)methyl)(tert-butoxycarbonyl)amino)propyl)-4-fluorophenyl)amino)-5-(trifluoromethyl)-benzoic acid 104d (735 mg, 82% yield) as a dark yellow solid. HPLC/MS 1.05 min (B), [M+H]⁺ 593.3. ¹H NMR (CD₃OD, 400 MHz): δ 1.35 (s, 9H), 1.65-1.84 (m, 2H), 2.46-2.62 (m, 2H), 3.26-3.32 (m, 2H), 3.73 (s, 3H), 4.36 (br s, 2H), 6.46-6.57 (m, 1H), 6.66 (d, J=8.80 Hz, 1H), 6.97-7.07 (m, 2H), 7.10 (d, J=8.80 Hz, 1H), 7.27 (dd, J=9.29, 5.38 Hz, 1H), 7.47 (dd, J=8.80, 1.96 Hz, 1H), 8.23 (d, J=1.47 Hz, 1H).

### Step E: tert-Butyl 8-fluoro-16-methoxy-20-oxo-2-(trifluoromethyl)-5,11,12,14,19,20-hexahydrodibenzo[b,m]pyrido[3,2-f][1,5,9]triazacyclotetradecine-13(10H)-carboxylate 104e

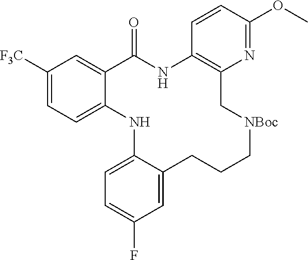 [SMILES: COc1ccc2c(n1)CN(C(=O)OC(C)(C)C)CCCc1cc(F)ccc1Nc1ccc(C(F)(F)F)cc1C(=O)N2]

Following the procedure outlined in Example 44, Step E, stirring the reaction mixture at room temperature for 1 h, tert-butyl 8-fluoro-16-methoxy-20-oxo-2-(trifluoromethyl)-5,11,12,14,19,20-hexahydrodibenzo[b,m]pyrido[3,2-f][1,5,9]triazacyclotetradecine-13(10)-carboxylate 104e (921 mg, 100% yield) was prepared as a brown solid. HPLC/MS 1.52 min (B), [M+H]⁺ 575.3 ¹H NMR (CD₃OD, 400 MHz): δ 1.51 (s, 9H) 1.58-1.71 (m, 2H), 2.45-2.55 (m, 2H), 3.12-3.20 (m, 2H), 3.91 (s, 3H), 4.34-4.51 (m, 2H), 6.81 (d, J=8.31 Hz, 1H), 6.99-7.09 (m, 2H), 7.49-7.58 (m, 1H), 7.66 (d, J=8.31 Hz, 3H), 8.06 (s, 1H).

### Step F: tert-Butyl 3⁴-fluoro-1⁶-methoxy-2⁴-oxo-2⁶-(trifluoromethyl)-2¹,2²,2³ 2⁴-tetra-hydro-7-aza-2(3,1)-quinazolina-1(3,2)-pyridina-3(1,2)-benzenacyclooctaphane-7-carboxylate 104f

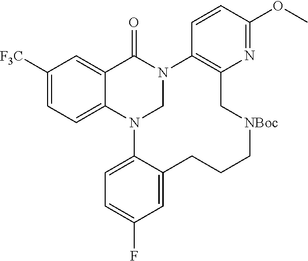 [SMILES: COc1ccc2c(n1)CN(C(=O)OC(C)(C)C)CCCc1cc(F)ccc1N1CN2C(=O)c2cc(C(F)(F)F)ccc21]

Following the procedure outlined in Example 27, Step E, stirring the reaction mixture at 90° C. for 16 h, tert-butyl 3⁴-fluoro-1⁶-methoxy-2⁴-oxo-2⁶-(trifluoromethyl)-2¹,2²,2³2⁴-tetrahydro-7-aza-2(3,1)-quinazolina-1(3,2)-pyridina-3(1,2)-benzenacyclooctaphane-7-carboxylate 104f was prepared as a dark brown solid. HPLC/MS 1.49 min (B), [M+H]⁺ 587.3. ¹H NMR (CD₃OD, 400 MHz): δ 1.43 (s, 9H), 1.52-1.62 (m, 1H), 1.76-1.88 (m, 1H), 2.44-2.55 (m, 1H), 2.74-2.87 (m, 2H), 3.53-3.63 (m, 1H), 3.91 (s, 3H), 4.59 (s, 1H), 4.68-4.74 (m, 1H), 4.79 (d, J=11.25 Hz, 1H), 5.71 (d, J=11.25 Hz, 1H), 6.46 (d, J=8.31 Hz, 1H), 6.81 (d, J=8.80 Hz, 1H), 7.10-7.18 (m, 1H), 7.25 (dd, J=9.78, 2.93 Hz, 1H), 7.36 (dd, J=8.56, 5.14 Hz, 1H), 7.65 (d, J=8.80 Hz, 2H), 8.30 (d, J=1.96 Hz, 1H).

### Step G: 3⁴-Fluoro-2⁶-(trifluoromethyl)-1¹,1²,2¹,2²,2³ 2⁴-hexahydro-7-aza-2(3,1)-quinazolina-1(5,6)-pyridina-3(1,2)-benzenacyclooctaphane-1²,2⁴-dione

### Example 104

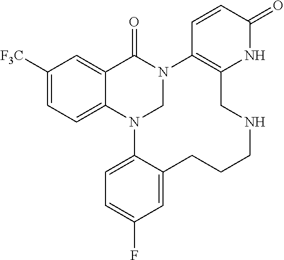 [SMILES: O=C1c2cc(C(F)(F)F)ccc2N2CN1c1ccc(=O)[nH]c1CNCCCc1cc(F)ccc12]

Following the procedure outlined in Example 27, Step E, stirring the reaction mixture at 90° C. for 19 h, 3⁴-fluoro-2⁶-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione (2.2 mg, 34% yield) was prepared as an off-white solid. HPLC/MS 1.04 min (B), [M+H]⁺ 473.2. ¹H NMR (CD₃OD, 400 MHz): δ 1.68-1.83 (m, 1H), 1.95-2.10 (m, 1H), 2.41-2.62 (m, 3H), 2.77-2.90 (m, 1H), 3.49 (d, J=13.69 Hz, 1H), 3.84 (d, J=13.69 Hz, 1H), 4.98 (d, J=9.78 Hz, 1H), 5.65 (d, J=10.27 Hz, 1H), 6.43 (d, J=8.80 Hz, 1H), 6.50 (d, J=9.78 Hz, 1H), 7.10 (br d, J=2.93 Hz, 1H), 7.20 (dd, J=9.29, 2.93 Hz, 1H), 7.33 (dd, J=8.80, 5.38 Hz, 1H), 7.56-7.63 (m, 2H), 8.23 (d, J=2.45 Hz, 1H).

### Example 105

2⁶,3⁴-Difluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride

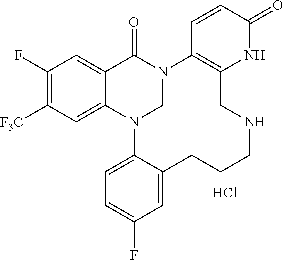 [SMILES: O=C1c2cc(F)c(C(F)(F)F)cc2N2CN1c1ccc(=O)[nH]c1CNCCCc1cc(F)ccc12 ; Cl]

### Step A: Methyl 2-((2-(3-((tert-butoxycarbonyl)amino)propyl)-4-fluorophenyl)amino)-5-fluoro-4-(trifluoromethyl)benzoate 105a

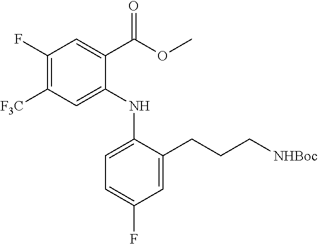 [SMILES: COC(=O)c1cc(F)c(C(F)(F)F)cc1Nc1ccc(F)cc1CCCNC(=O)OC(C)(C)C]

Following the procedure outlined in Example 3, Step A, substituting 2-bromo-4-fluoroaniline with Int-3g, methyl 2-bromo-5-(trifluoromethyl)benzoate Int-2 with Int-2e, BINAP with XantPhos, Pd₂(dba)₃ with Pd(OAc)₂, toluene with dioxane and heating to 80° C. for 21 h, methyl 2-((2-(3-((tert-butoxycarbonyl)amino)propyl)-4-fluorophenyl)amino)-5-fluoro-4-(trifluoromethyl)benzoate 105a (1.96 g, 61% yield) was prepared as a white solid. HPLC/MS 1.54 min (B), [M+H]⁺ 488.2. ¹H NMR (CDCl₃, 400 MHz): δ 1.40 (s, 9H), 1.68-1.80 (m, 2H), 2.53-2.64 (m, 2H), 3.06-3.21 (m, 2H), 3.96 (s, 3H), 4.54-4.64 (m, 1H), 6.87 (d, J=5.87 Hz, 1H), 6.92-6.99 (m, 1H), 7.00-7.06 (m, 1H), 7.16-7.23 (m, 1H), 7.77 (d, J=10.76 Hz, 1H), 9.11 (s, 1H).

### Step B: Methyl 2-((2-(3-aminopropyl)-4-fluorophenyl)amino)-5-fluoro-4-(trifluoro-methyl)benzoate, trifluoroacetic acid salt 105b

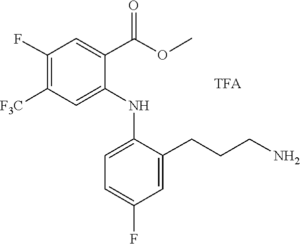 [SMILES: COC(=O)c1cc(F)c(C(F)(F)F)cc1Nc1ccc(F)cc1CCCN]

Following the procedure outlined in Example 104, Step A, after 18 h methyl 2-((2-(3-aminopropyl)-4-fluorophenyl)amino)-5-fluoro-4-(trifluoromethyl)benzoate, trifluoroacetic acid salt 105b (2.30 g, 100% yield) was prepared as a light brown solid. HPLC/MS 0.95 min (A), [M+H]⁺ 389.1. ¹H NMR (CD₃OD, 400 MHz): δ 1.95 (br t, J=7.83 Hz, 2H), 2.66-2.79 (m, 2H), 2.95 (s, 2H), 3.99 (s, 3H), 6.83 (d, J=5.87 Hz, 1H), 7.12 (td, J=8.56, 2.93 Hz, 1H), 7.21 (dd, J=9.54, 3.18 Hz, 1H), 7.34 (dd, J=8.80, 5.38 Hz, 1H), 7.88 (d, J=11.25 Hz, 1H).

### Step C: Methyl 2-((2-(3-((tert-butoxycarbonyl)((3-(1,3-dioxoisoindolin-2-yl)-6-methoxy-pyridin-2-yl)methyl)amino)propyl)-4-fluorophenyl)amino)-5-fluoro-4-(trifluoromethyl)-benzoate 105c

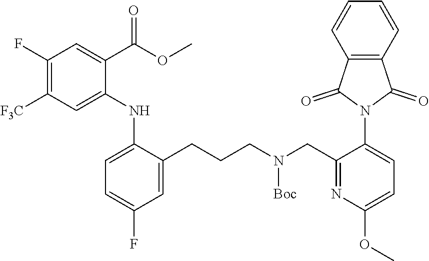 [SMILES: COC(=O)c1cc(F)c(C(F)(F)F)cc1Nc1ccc(F)cc1CCCN(Cc1nc(OC)ccc1N1C(=O)c2ccccc2C1=O)C(=O)OC(C)(C)C]

Following the preparation outlined in Example 44, Step B, substituting methyl 5-fluoro-2-((4-fluoro-2-formylphenyl)amino)-4-(trifluoromethyl)benzoate with 3-(1,3-dioxoisoindolin-2-yl)-6-methoxypicolinaldehyde Int-1g and 2-(6-methoxy-3-nitropyridin-2-yl)ethan-1-amine with 105b, methyl 2-((2-(3-((tert-butoxycarbonyl)((3-(1,3-dioxoisoindolin-2-yl)-6-methoxy-pyridin-2-yl)methyl)amino)propyl)-4-fluorophenyl)amino)-5-fluoro-4-(trifluoromethyl)-benzoate 105c (1.21 g, 71% yield) was prepared as a light yellow solid. HPLC/MS 1.70 min (B), [M+H]⁺ 755.4. ¹H NMR (CD₃OD, 400 MHz): δ 1.10-1.23 (m, 9H), 1.29-1.35 (m, 2H), 1.58-1.84 (m, 2H), 2.43-2.61 (m, 2H), 3.13-3.24 (m, 1H), 3.89 (s, 6H), 4.24-4.45 (m, 2H), 6.72-6.86 (m, 2H), 6.98-7.16 (m, 2H), 7.21-7.30 (m, 1H), 7.57 (d, J=8.80 Hz, 1H), 7.92 (br s, 5H), 9.15 (s, 1H).

### Step D: 2-((2-(3-((tert-Butoxycarbonyl)((3-(1,1-dihydroxy-3-oxoisoindolin-2-yl)-6-methoxypyridin-2-yl)methyl)amino)propyl)-4-fluorophenyl)amino)-5-fluoro-4-(trifluoromethyl)-benzoic acid 105d

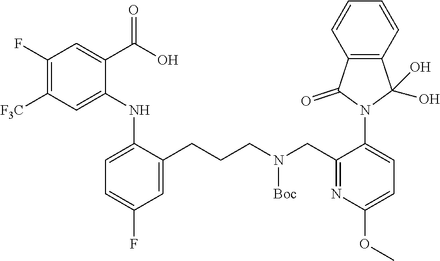 [SMILES: COc1ccc(N2C(=O)c3ccccc3C2(O)O)c(CN(CCCc2cc(F)ccc2Nc2cc(C(F)(F)F)c(F)cc2C(=O)O)C(=O)OC(C)(C)C)n1]

Following the procedure outlined in Example 27, Step B, stirring the reaction mixture at RT overnight, 2-((2-(3-((tert-butoxycarbonyl)((3-(1,1-dihydroxy-3-oxoisoindolin-2-yl)-6-methoxypyridin-2-yl)methyl)amino)propyl)-4-fluorophenyl)amino)-5-fluoro-4-(trifluoromethyl)-benzoic acid 105d (1.27 g, 100% yield) was prepared as a foamy yellow solid. HPLC/MS 0.92 min (B), [M+H]⁺ 759.4. ¹H NMR (CD₃OD, 400 MHz): δ 1.28 (s, 9H), 1.84 (ddd, J=8.56, 3.18, 1.47 Hz, 2H), 2.47-2.72 (m, 2H), 3.34-3.43 (m, 2H), 3.82 (s, 3H), 4.44-4.64 (m, 2H), 6.63-6.88 (m, 2H), 6.95-7.15 (m, 2H), 7.27 (dd, J=8.80, 5.38 Hz, 1H), 7.54-7.70 (m, 3H), 7.74-7.86 (m, 1H), 8.03 (dt, J=7.83, 0.98 Hz, 2H).

### Step E: 2-((2-(3-(((3-Amino-6-methoxypyridin-2-yl)methyl)(tert-butoxycarbonyl)-amino)propyl)-4-fluorophenyl)amino)-5-fluoro-4-(trifluoromethyl)benzoic acid 105e

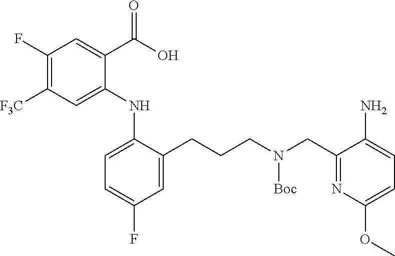 [SMILES: COc1ccc(N)c(CN(CCCc2cc(F)ccc2Nc2cc(C(F)(F)F)c(F)cc2C(=O)O)C(=O)OC(C)(C)C)n1]

Following the procedure outlined in Example 104, Step D, stirring at 80° C., 2-((2-(3-(((3-amino-6-methoxypyridin-2-yl)methyl)(tert-butoxycarbonyl)amino)propyl)-4-fluorophenyl)-amino)-5-fluoro-4-(trifluoromethyl)benzoic acid 105e (994 mg, 100% yield) was prepared as a white solid. HPLC/MS 1.07 min (B), [M+H]⁺ 611.3. ¹H NMR (CD₃OD, 400 MHz): δ 1.36 (s, 9H), 1.68-1.86 (m, 2H), 2.49-2.63 (m, 2H), 3.25-3.32 (m, 2H), 3.72 (s, 3H), 4.37 (s, 2H), 6.50 (br d, J=7.83 Hz, 1H), 6.78 (d, J=5.87 Hz, 1H), 6.90-7.04 (m, 2H), 7.09 (d, J=8.31 Hz, 1H), 7.24 (dd, J=8.80, 5.38 Hz, 1H), 7.81 (d, J=11.74 Hz, 1H).

### Step F: tert-Butyl 2,8-difluoro-16-methoxy-20-oxo-3-(trifluoromethyl)-5,11,12,14,19,20-hexahydrodibenzo[b,m]pyrido[3,2-f][1,5,9]triazacyclotetradecine-13(10H)-carboxylate 105f

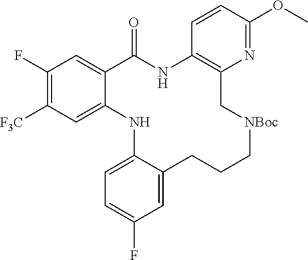 [SMILES: COc1ccc2c(n1)CN(C(=O)OC(C)(C)C)CCCc1cc(F)ccc1Nc1cc(C(F)(F)F)c(F)cc1C(=O)N2]

Following the procedure outlined in Example 44, Step E, stirring the reaction mixture at room temperature for 2 h, tert-butyl 2,8-difluoro-16-methoxy-20-oxo-3-(trifluoromethyl)-5,11,12,14,19,20-hexahydrodibenzo[b,m]pyrido[3,2-f][1,5,9]triazacyclotetradecine-13(10H)-carboxylate 105f (976 mg, 100% yield) was prepared as a brown solid. HPLC/MS 1.53 min (B), [M+H]⁺ 592.3 ¹H NMR (CD₃OD, 400 MHz): δ 1.51 (s, 9H), 1.68-1.79 (m, 2H), 2.54-2.64 (m, 2H), 3.07-3.14 (m, 2H), 3.90 (s, 3H), 4.35-4.52 (m, 2H), 6.77 (d, J=8.31 Hz, 1H), 6.99 (dt, J=5.87, 2.93 Hz, 2H), 7.21 (dd, J=8.56, 5.14 Hz, 1H), 7.60-7.75 (m, 3H), 8.00 (s, 1H).

### Step G: tert-Butyl 2⁶,3⁴-difluoro-1⁶-methoxy-2⁴-oxo-2⁷-(trifluoromethyl)-2¹,2²,2³ 2⁴-tetrahydro-7-aza-2(3,1)-quinazolina-1(3,2)-pyridina-3(1,2)-benzenacyclooctaphane-7-carboxylate 105g

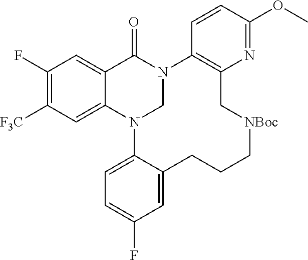 [SMILES: COc1ccc2c(n1)CN(C(=O)OC(C)(C)C)CCCc1cc(F)ccc1N1CN2C(=O)c2cc(F)c(C(F)(F)F)cc21]

Following the procedure outlined in Example 27, Step E, stirring the reaction mixture at 90° C. for 16 h, tert-butyl 2⁶,3⁴-difluoro-1⁶-methoxy-2⁴-oxo-2⁷-(trifluoromethyl)-2¹,2²,2³,2⁴-tetrahydro-7-aza-2(3,1)-quinazolina-1(3,2)-pyridina-3(1,2)-benzenacyclooctaphane-7-carboxylate 105g (177 mg, 62% yield) was prepared as a dark brown solid. HPLC/MS 1.49 min (B), [M+H]⁺ 605.3. ¹H NMR (CD₃OD, 400 MHz): δ 1.45 (s, 9H), 1.51 (s, 1H), 1.75-1.88 (m, 1H), 2.44-2.56 (m, 1H), 2.72-2.89 (m, 2H), 3.52-3.65 (m, 1H), 3.91 (s, 3H), 4.55-4.65 (m, 1H), 4.69-4.75 (m, 1H), 4.77 (d, J=11.25 Hz, 1H), 5.68 (d, J=10.76 Hz, 1H), 6.47 (d, J=5.38 Hz, 1H), 6.80 (d, J=8.80 Hz, 1H), 7.16 (td, J=8.31, 2.93 Hz, 1H), 7.27 (dd, J=9.29, 2.93 Hz, 1H), 7.38 (dd, J=8.56, 5.14 Hz, 1H), 7.64 (d, J=8.80 Hz, 1H), 7.93 (d, J=10.27 Hz, 1H).

### Step H: 2⁶,3⁴-Difluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride

### Example 105

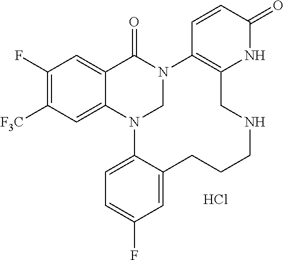 [SMILES: O=C1c2cc(F)c(C(F)(F)F)cc2N2CN1c1ccc(=O)[nH]c1CNCCCc1cc(F)ccc12 ; Cl]

Following the procedure outlined in Example 27, Step F, stirring the reaction mixture at 90° C. for 17 h, 2⁶,3⁴-difluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride (58 mg, 41% yield) was prepared as an off white solid. HPLC/MS 0.69 min (G), [M+H]⁺ 491.1. ¹H NMR (CD₃OD, 400 MHz): δ 1.67-1.84 (m, 1H), 1.94-2.09 (m, 1H), 2.55-2.67 (m, 1H), 2.70-2.82 (m, 1H), 3.05-3.20 (m, 2H), 4.07 (d, J=8.80 Hz, 2H), 4.77-4.81 (m, 1H), 5.68 (d, J=11.25 Hz, 1H), 6.48 (d, J=5.38 Hz, 1H), 6.76 (d, J=9.29 Hz, 1H), 7.13-7.27 (m, 2H), 7.40 (dd, J=8.56, 5.14 Hz, 1H), 7.69 (d, J=8.80 Hz, 1H), 7.88 (d, J=10.27 Hz, 1H).

### Example 106

### 2⁶,3³,3⁴-Trifluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride

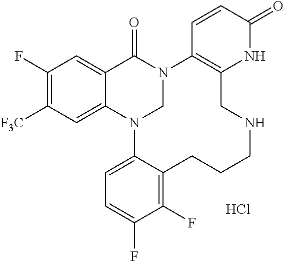 [SMILES: O=C1c2cc(F)c(C(F)(F)F)cc2N2CN1c1ccc(=O)[nH]c1CNCCCc1c2ccc(F)c1F ; Cl]

### Step A: Methyl 2-((2-(3-((tert-butoxycarbonyl)amino)propyl)-3,4-difluorophenyl)-amino)-5-fluoro-4-(trifluoromethyl)benzoate 106a

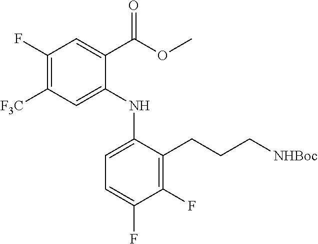 [SMILES: COC(=O)c1cc(F)c(C(F)(F)F)cc1Nc1ccc(F)c(F)c1CCCNC(=O)OC(C)(C)C]

Following the procedure outlined in Example 3, Step A, substituting 2-bromo-4-fluoroaniline with Int-3i, methyl 2-bromo-5-(trifluoromethyl)benzoate Int-2 with Int-2e, BINAP with XantPhos, Pd₂(dba)₃ with Pd(OAc)₂, toluene with dioxane and heating to 80° C. for 21 h, methyl 2-((2-(3-((tert-butoxycarbonyl)amino)propyl)-3,4-difluorophenyl)amino)-5-fluoro-4-(trifluoromethyl)benzoate 106a (967 mg, 59% yield) was prepared as an off-white solid. HPLC/MS 1.55 min (B), [M+H]⁺ 507.2. ¹H NMR (CDCl₃, 400 MHz): δ 1.40 (s, 9H), 1.67-1.79 (m, 2H), 2.62-2.75 (m, 2H), 3.10-3.23 (m, 2H), 3.97 (s, 3H), 4.61-4.72 (m, 1H), 6.97 (d, J=5.38 Hz, 3H), 7.79 (d, J=10.76 Hz, 1H), 9.17-9.25 (m, 1H).

### Step B: Methyl 2-((2-(3-aminopropyl)-3,4-difluorophenyl)amino)-5-fluoro-4-(trifluoro-methyl)benzoate, trifluoroacetic acid salt 106b

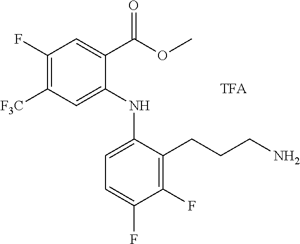 [SMILES: COC(=O)c1cc(F)c(C(F)(F)F)cc1Nc1ccc(F)c(F)c1CCCN]

Following the procedure outlined in Example 104, Step A, after 18 h, methyl 2-((2-(3-aminopropyl)-3,4-difluorophenyl)amino)-5-fluoro-4-(trifluoromethyl)benzoate, trifluoroacetic acid salt 106b (0.899 g, 100%) was prepared as an off white solid. HPLC/MS 0.94 min (A), [M+H]⁺ 407.2. ¹H NMR (CD₃OD, 400 MHz): δ 1.87-1.98 (m, 2H), 2.75-2.86 (m, 2H), 2.92-3.02 (m, 2H), 3.98 (s, 3H), 6.95 (d, J=5.87 Hz, 1H), 7.15-7.21 (m, 1H), 7.26 (dd, J=9.78, 8.80 Hz, 1H), 7.89 (d, J=11.25 Hz, 1H).

### Step C: Methyl 2-((2-(3-((tert-butoxycarbonyl)((3-(1,3-dioxoisoindolin-2-yl)-6-methoxy-pyridin-2-yl)methyl)amino)propyl)-3,4-difluorophenyl)amino)-5-fluoro-4-(trifluoromethyl)-benzoate 106c

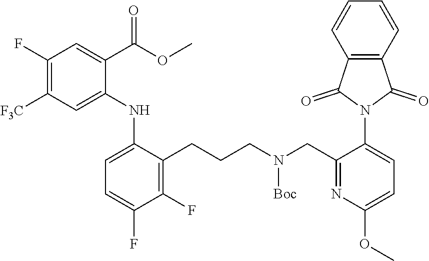 [SMILES: COC(=O)c1cc(F)c(C(F)(F)F)cc1Nc1ccc(F)c(F)c1CCCN(Cc1nc(OC)ccc1N1C(=O)c2ccccc2C1=O)C(=O)OC(C)(C)C]

Following the preparation outlined in Example 44, Step B, substituting methyl 5-fluoro-2-((4-fluoro-2-formylphenyl)amino)-4-(trifluoromethyl)benzoate with 3-(1,3-dioxoisoindolin-2-yl)-6-methoxypicolinaldehyde Int-1g and 2-(6-methoxy-3-nitropyridin-2-yl)ethan-1-amine with 106b, methyl 2-((2-(3-((tert-butoxycarbonyl)((3-(1,3-dioxoisoindolin-2-yl)-6-methoxy-pyridin-2-yl)methyl)amino)propyl)-3,4-difluorophenyl)amino)-5-fluoro-4-(trifluoromethyl)-benzoate 106c (682 mg, 89% yield) was prepared as a light yellow solid. HPLC/MS 1.70 min (B), [M+H]⁺ 773.4. ¹H NMR (CDCl₃, 400 MHz): δ 1.12-1.22 (m, 9H), 1.63-1.71 (m, 1H), 1.72-1.81 (m, 1H), 2.50-2.69 (m, 2H), 3.16-3.26 (m, 1H), 3.35-3.47 (m, 1H), 3.84-3.94 (m, 6H), 4.22-4.33 (m, 1H), 4.35-4.45 (m, 1H), 6.73 (d, J=8.31 Hz, 1H), 6.86-6.93 (m, 1H), 6.94-7.09 (m, 2H), 7.33-7.43 (m, 1H), 7.65-7.85 (m, 3H), 7.85-7.98 (m, 2H), 9.15 (s, 1H).

### Step D: 2-((2-(3-((tert-Butoxycarbonyl)((3-(1,1-dihydroxy-3-oxoisoindolin-2-yl)-6-methoxypyridin-2-yl)methyl)amino)propyl)-3,4-difluorophenyl)amino)-5-fluoro-4-(trifluoro-methyl)benzoic acid 106d

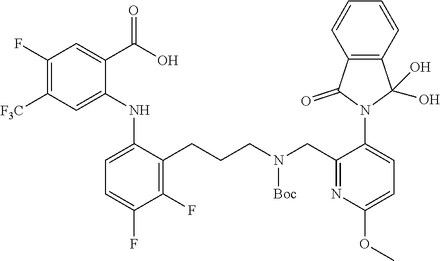 [SMILES: COc1ccc(N2C(=O)c3ccccc3C2(O)O)c(CN(CCCc2c(Nc3cc(C(F)(F)F)c(F)cc3C(=O)O)ccc(F)c2F)C(=O)OC(C)(C)C)n1]

Following the procedure outlined in Example 27, Step B, stirring the reaction mixture at RT for 2 h, 2-((2-(3-((tert-butoxycarbonyl)((3-(1,1-dihydroxy-3-oxoisoindolin-2-yl)-6-methoxypyridin-2-yl)methyl)amino)propyl)-3,4-difluorophenyl)amino)-5-fluoro-4-(trifluoro-methyl)benzoic acid 106d (740 mg, 100% yield) was prepared as a foamy yellow solid. HPLC/MS 0.93 min (B), [M+H]⁺ 777.4. ¹H NMR (CD₃OD, 400 MHz): δ 1.25 (s, 9H), 1.73-1.83 (m, 2H), 2.54-2.80 (m, 2H), 3.32-3.41 (m, 2H), 3.81 (s, 3H), 4.38-4.63 (m, 2H), 6.70 (br d, J=8.31 Hz, 1H), 6.85-6.98 (m, 1H), 7.08-7.24 (m, 2H), 7.54-7.72 (m, 3H), 7.83 (d, J=11.25 Hz, 1H), 8.03 (br d, J=7.34 Hz, 2H).

### Step E: 2-((2-(3-(((3-Amino-6-methoxypyridin-2-yl)methyl)(tert-butoxycarbonyl)-amino)propyl)-3,4-difluorophenyl)amino)-5-fluoro-4-(trifluoromethyl)benzoic acid 106e

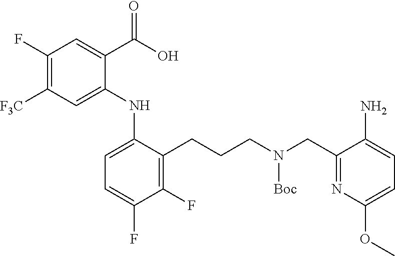 [SMILES: COc1ccc(N)c(CN(CCCc2c(Nc3cc(C(F)(F)F)c(F)cc3C(=O)O)ccc(F)c2F)C(=O)OC(C)(C)C)n1]

Following the procedure outlined in Example 104, Step D, stirring at 80° C., 2-((2-(3-(((3-amino-6-methoxypyridin-2-yl)methyl)(tert-butoxycarbonyl)amino)propyl)-3,4-difluoro-phenyl)-amino)-5-fluoro-4-(trifluoromethyl)benzoic acid 106e (431 mg, 82% yield) was prepared as a light brown solid. HPLC/MS 1.12 min (B), [M+H]⁺ 629.3. ¹H NMR (CD₃OD, 400 MHz): δ 1.35 (s, 9H), 1.66-1.80 (m, 2H), 2.55-2.67 (m, 2H), 3.22-3.29 (m, 2H), 3.71 (s, 3H), 4.35 (s, 2H), 6.48 (d, J=8.80 Hz, 1H), 6.89 (d, J=5.87 Hz, 1H), 7.09 (br d, J=8.80 Hz, 2H), 7.13-7.21 (m, 1H), 7.84 (d, J=11.25 Hz, 1H).

### Step F: tert-Butyl 2,8,9-trifluoro-16-methoxy-20-oxo-3-(trifluoromethyl)-5,11,12,14,19,20-hexahydrodibenzo[b,m]pyrido[3,2-f][1,5,9]triazacyclotetradecine-13(10H)-carboxylate 106f

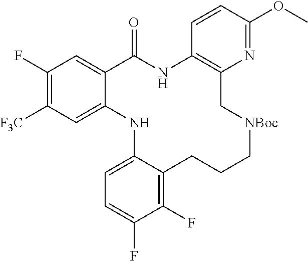 [SMILES: COc1ccc2c(n1)CN(C(=O)OC(C)(C)C)CCCc1c(ccc(F)c1F)Nc1cc(C(F)(F)F)c(F)cc1C(=O)N2]

Following the procedure outlined in Example 44, Step E, stirring the reaction mixture at room temperature for 1 h, tert-butyl 2,8,9-trifluoro-16-methoxy-20-oxo-3-(trifluoromethyl)-5,11,12,14,19,20-hexahydrodibenzo[b,m]pyrido[3,2-f][1,5,9]triazacyclotetradecine-13(10)-carboxylate 106f (532 mg, 91% yield) was prepared as a brown solid. HPLC/MS 1.54 min (B), [M+H]⁺ 611.3 ¹H NMR (CD₃OD, 400 MHz): δ 1.49 (s, 9H), 1.64-1.75 (m, 2H), 2.62-2.73 (m, 2H), 3.11-3.18 (m, 2H), 3.88 (s, 3H), 4.36 (s, 2H), 6.75 (d, J=8.80 Hz, 1H), 6.87-6.95 (m, 1H), 7.03-7.14 (m, 1H), 7.63 (d, J=8.80 Hz, 3H), 7.98 (s, 2H).

### Step G: tert-Butyl 2⁶,3³,3⁴-trifluoro-1⁶-methoxy-2⁴-oxo-2⁷-(trifluoromethyl)-2¹,2²,2³ 2⁴-tetrahydro-7-aza-2(3,1)-quinazolina-1(3,2)-pyridina-3(1,2)-benzenacyclooctaphane-7-carboxylate 106g

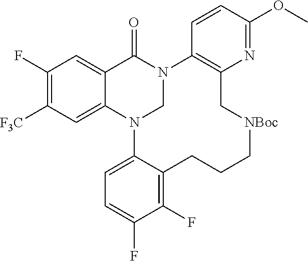 [SMILES: COc1ccc2c(n1)CN(C(=O)OC(C)(C)C)CCCc1c(ccc(F)c1F)N1CN2C(=O)c2cc(F)c(C(F)(F)F)cc21]

Following the procedure outlined in Example 27, Step E, stirring the reaction mixture at 90° C. for 18 h, tert-butyl 2⁶,3³,3⁴-trifluoro-1⁶-methoxy-2⁴-oxo-2⁷-(trifluoromethyl)-2¹,2²,2³,2⁴-tetrahydro-7-aza-2(3,1)-quinazolina-1(3,2)-pyridina-3(1,2)-benzenacyclooctaphane-7-carboxylate 106g (68 mg, 37% yield) was prepared as a light grey solid. HPLC/MS 1.54 min (B), [M+H]⁺ 623.3. ¹H NMR (CD₃OD, 400 MHz): δ 1.42 (s, 9H) 1.49-1.61 (m, 1H) 1.77-1.92 (m, 1H) 2.58-2.69 (m, 1H) 2.71-2.88 (m, 2H) 3.52-3.63 (m, 1H) 3.90 (s, 3H) 4.62-4.72 (m, 3H) 5.66 (d, J=11.25 Hz, 1H) 6.47 (d, J=5.38 Hz, 1H) 6.79 (d, J=8.31 Hz, 1H) 7.18-7.26 (m, 1H) 7.28-7.38 (m, 1H) 7.62 (d, J=8.80 Hz, 1H) 7.93 (d, J=10.27 Hz, 1H).

### Step H: 2⁶,3³,3⁴-Trifluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride

### Example 106

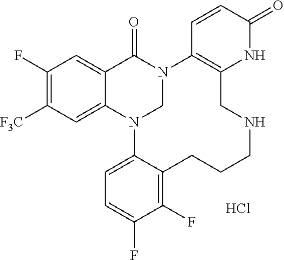 [SMILES: O=C1c2cc(F)c(C(F)(F)F)cc2N2CN1c1ccc(=O)[nH]c1CNCCCc1c2ccc(F)c1F ; Cl]

Following the procedure outlined in Example 27, Step F, stirring the reaction mixture at 90° C. for 15 h, 2⁶,3³,3⁴-trifluoro-2⁷-(trifluoromethyl)-1¹,1²,2¹,2²,2³,2⁴-hexahydro-7-aza-2(3,1)-quinazolina-1(5,6)-pyridina-3(1,2)-benzenacyclooctaphane-1²,2⁴-dione hydrochloride (31 mg, 57% yield) was prepared as a white solid. HPLC/MS 0.81 min (A), [M+H]⁺ 509.2. ¹H NMR (CD₃OD, 400 MHz): δ 1.57-1.71 (m, 1H), 1.90-2.06 (m, 1H), 2.48-2.62 (m, 1H), 2.81-2.94 (m, 1H), 3.02-3.11 (m, 1H), 3.15 (br d, J=6.36 Hz, 1H), 3.95-4.16 (m, 2H), 4.70-4.74 (m, 1H), 5.60 (d, J=10.76 Hz, 1H), 6.45 (d, J=5.87 Hz, 1H), 6.72 (d, J=8.80 Hz, 1H), 7.16-7.23 (m, 1H), 7.25-7.35 (m, 1H), 7.63 (d, J=9.29 Hz, 1H), 7.84 (d, J=9.78 Hz, 1H).

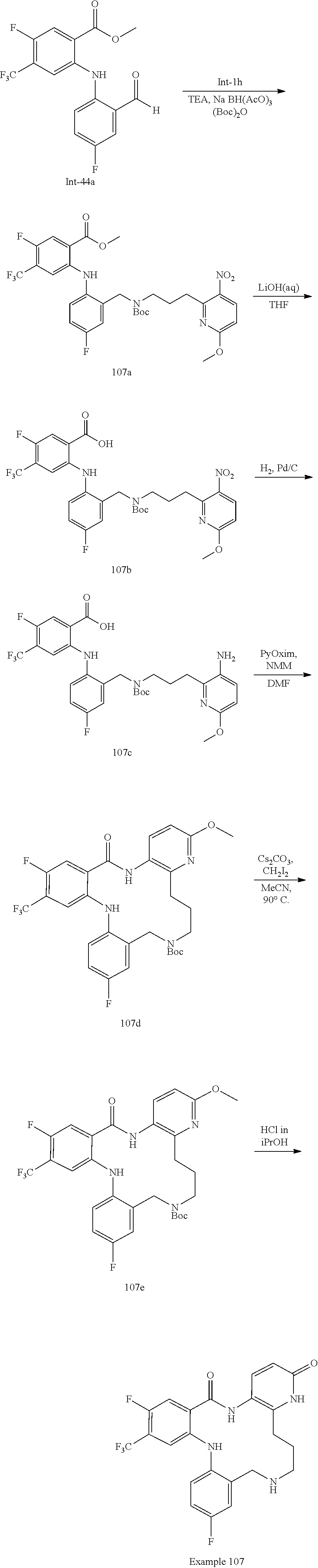

### Example 107

2⁶,3⁴-Difluoro-2⁷-(trifluoromhethyly)-1¹,1²,2¹,2²2³,2⁴-hexahydro-5-aza-2(3,1)-quinazolina-1(5,6)-pyridina-3(1,2)-benzenacyclooctaphane-1²,2⁴-dione hydrochloride

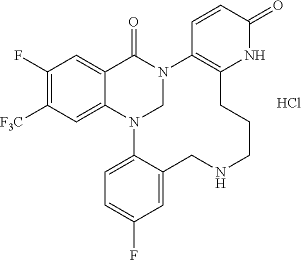 [SMILES: O=C1c2cc(F)c(C(F)(F)F)cc2N2CN1c1ccc(=O)[nH]c1CCCNCc1cc(F)ccc12 ; Cl]

### Step A: Methyl 2-((2-(((tert-butoxycarbonyl)(3-(6-methoxy-3-nitropyridin-2-yl)propyl)-amino)methyl)-4-fluorophenyl)amnio)-5-fluoro-4-(trifluoromethyl)benzoate 107a

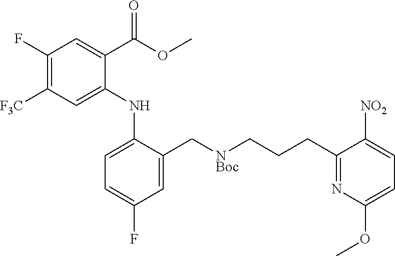 [SMILES: COC(=O)c1cc(F)c(C(F)(F)F)cc1Nc1ccc(F)cc1CN(CCCc1nc(OC)ccc1[N+](=O)[O-])C(=O)OC(C)(C)C]

Following the preparation outlined in Example 44, Step B, substituting 2-(6-methoxy-3-nitropyridin-2-yl)ethan-1-amine with Int-1h, methyl 2-((2-(((tert-butoxycarbonyl)(3-(6-methoxy-3-nitropyridin-2-yl)propyl)amino)methyl)-4-fluorophenyl)amino)-5-fluoro-4-(trifluoromethyl) benzoate 107a (801 mg, 85% yield) was prepared as a yellow solid. HPLC/MS 1.61 min (B), [M+H]⁺ 655.2. ¹H NMR (CDCl₃, 400 MHz): δ 9.05 (br s, 1H), 8.25 (d, 1H, J=8.8 Hz), 7.7-7.9 (m, 1H), 7.23 (br d, 1H, J=5.4 Hz), 7.0-7.1 (m, 2H), 6.7-6.9 (m, 1H), 6.6-6.7 (m, 1H), 4.3-4.5 (m, 2H), 3.99 (s, 6H), 3.2-3.5 (m, 2H), 3.08 (br t, 2H, J=6.8 Hz), 1.9-2.1 (m, 2H), 1.3-1.5 (m, 11H).

### Step B: 2-((2-(((tert-Butoxycarbonyl)(3-(6-methoxy-3-nitropyridin-2-yl)propyl)-amino)-methyl)-4-fluorophenyl)amino)-5-fluoro-4-(trifluoromethyl)benzoic acid 107b

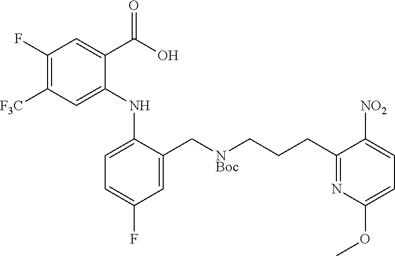 [SMILES: COc1ccc([N+](=O)[O-])c(CCCN(Cc2cc(F)ccc2Nc2cc(C(F)(F)F)c(F)cc2C(=O)O)C(=O)OC(C)(C)C)n1]

Following the procedure outlined in Example 27, Step B, stirring the reaction mixture at 50° C. for 2.5 h, 2-((2-(((tert-butoxycarbonyl)(3-(6-methoxy-3-nitropyridin-2-yl)propyl)-amino)methyl)-4-fluorophenyl)amino)-5-fluoro-4-(trifluoromethyl)benzoic acid 107b (803 mg, 93% yield) was prepared as a yellow foam, in 90% purity. HPLC/MS 1.52 min (A), [M+H]⁺ 641.2. ¹H NMR (DMSO-d₆, 400 MHz): δ 13.5-14.1 (m, 1H), 9.18 (s, 1H), 8.32 (d, 1H, J=9.3 Hz), 7.8-8.0 (m, 1H), 7.3-7.4 (m, 1H), 7.21 (br d, 1H, J=2.9 Hz), 7.0-7.2 (m, 1H), 6.85 (d, 1H, J=8.8 Hz), 6.5-6.7 (m, 1H), 4.36 (br s, 2H), 3.89 (s, 3H), 3.3-3.4 (m, 3H), 2.8-3.0 (m, 2H), 1.7-1.9 (m, 2H), 1.26 (s, 9H).

### Step C: 2-((2-(((3-(3-Amino-6-methoxypyridin-2-yl)propyl)(tert-butoxycarbonyl)amino)-methyl)-4-fluorophenyl)amino)-5-fluoro-4-(trifluoromethyl)benzoic acid 107c

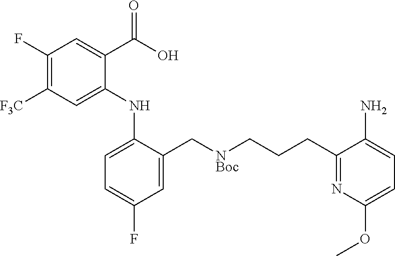 [SMILES: COc1ccc(N)c(CCCN(Cc2cc(F)ccc2Nc2cc(C(F)(F)F)c(F)cc2C(=O)O)C(=O)OC(C)(C)C)n1]

Following the procedure outlined in Example 44, Step D, 2-((2-(((3-(3-amino-6-methoxypyridin-2-yl)propyl)(tert-butoxycarbonyl)amino)methyl)-4-fluorophenyl)amino)-5-fluoro-4-(trifluoromethyl)benzoic acid 107c (350 mg, 40% yield) was prepared as a beige solid. HPLC/MS 1.09 min (A), [M+H]⁺ 611.2. ¹H NMR (DMSO-d₆, 400 MHz): δ 9.33 (br s, 1H), 7.7-7.9 (m, 1H), 7.3-7.4 (m, 1H), 7.1-7.2 (m, 1H), 6.9-7.1 (m, 2H), 6.6-6.7 (m, 1H), 6.37 (d, 1H, J=8.8 Hz), 4.36 (s, 2H), 3.63 (s, 3H), 3.1-3.3 (m, 2H), 2.4-2.5 (m, 2H), 1.8-1.9 (m, 2H), 1.1-1.4 (m, 19H).

### Step D: tert-Butyl 2,8-difluoro-16-methoxy-20-oxo-3-(trifluoromethyl)-5,12,13,14,19,20-hexahydrodibenzo[c,f]pyrido[3,2-j][1,5,9]triazacyclotetradecine-11(10H)-carboxylate 107d

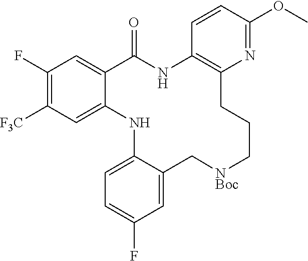 [SMILES: COc1ccc2c(n1)CCCN(C(=O)OC(C)(C)C)Cc1cc(F)ccc1Nc1cc(C(F)(F)F)c(F)cc1C(=O)N2]

Following the procedure outlined in Example 27, Step D, stirring the reaction mixture at room temperature for 24 h, tert-butyl 2,8-difluoro-16-methoxy-20-oxo-3-(trifluoromethyl)-5,12,13,14,19,20-hexahydrodibenzo[c,f]pyrido[3,2-j][1,5,9]triazacyclotetradecine-11(10)-carboxylate 107d (80.4 mg, 24% yield) was prepared as an off white solid. HPLC/MS 1.44 min (A), [M+H]⁺ 593.2. ¹H NMR (CDCl₃, 400 MHz): δ 10.66 (br s, 1H), 8.5-8.7 (m, 1H), 8.2-8.4 (m, 1H), 7.5-7.6 (m, 1H), 7.4-7.5 (m, 1H), 6.9-7.0 (m, 1H), 6.8-6.9 (m, 1H), 6.63 (d, 1H, J=8.8 Hz), 6.3-6.4 (m, 1H), 4.6-5.0 (m, 2H), 3.89 (s, 3H), 3.4-3.7 (m, 2H), 2.4-2.6 (m, 2H), 1.48 (s, 11H).

### Step E: tert-Butyl 2⁶,3⁴-difluoro-1⁶-methoxy-2⁴-oxo-27-(trifluoromethyl)-2¹,2²,2³ 2⁴-tetrahydro-5-aza-2(3,1)-quinazolina-1(3,2)-pyridina-3(1,2)-benzenacyclooctaphane-5-carboxylate 107e

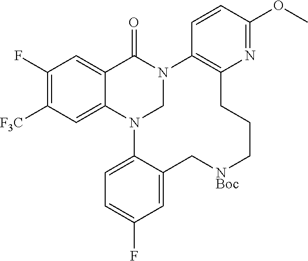 [SMILES: COc1ccc2c(n1)CCCN(C(=O)OC(C)(C)C)Cc1cc(F)ccc1N1CN2C(=O)c2cc(F)c(C(F)(F)F)cc21]

Following the procedure outlined in Example 27, Step E, stirring the reaction mixture at 90° C. for 20 h, tert-butyl 2⁶,3⁴-difluoro-1⁶-methoxy-2⁴-oxo-27-(trifluoromethyl)-2¹,2²,2³,2⁴-tetrahydro-5-aza-2(3,1)-quinazolina-1(3,2)-pyridina-3(1,2)-benzenacyclooctaphane-5-carboxylate 107e (101 mg, >100% yield) was prepared as a white solid. HPLC/MS 1.41 min (A), [M+H]⁺ 605.2. ¹H NMR (CDCl₃, 400 MHz): δ 7.99 (d, 1H, J=9.8 Hz), 7.42 (d, 1H, J=8.8 Hz), 7.2-7.3 (m, 2H), 7.1-7.2 (m, 1H), 6.68 (d, 1H, J=8.3 Hz), 6.5-6.6 (m, 1H), 5.72 (d, 1H, J=10.8 Hz), 4.8-4.9 (m, 1H), 4.67 (d, 1H, J=10.8 Hz), 4.25 (s, 1H), 3.95 (s, 3H), 3.3-3.6 (m, 1H), 2.8-3.0 (m, 1H), 2.6-2.7 (m, 2H), 2.0-2.2 (m, 1H), 1.8-2.0 (m, 1H), 1.54 (s, 9H).

### Step F: 2⁶,3⁴-Difluoro-2⁷-(trifluoromethyl)-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride

### Example 107

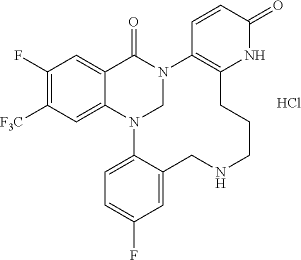 [SMILES: O=C1c2cc(F)c(C(F)(F)F)cc2N2CN1c1ccc(=O)[nH]c1CCCNCc1cc(F)ccc12 ; Cl]

Following the procedure outlined in Example 27, Step F, stirring the reaction mixture at 90° C. for 70 h, to which was added additional 5N HCl-ⁱPrOH (50 equiv) and the reaction mixture was stirred at 90° C. for 20 h, 2⁶,3⁴-difluoro-2⁷-(trifluoromethyl)-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride (63.3 mg, 75% yield) was prepared as an off white solid. HPLC/MS 0.65 min (A), [M+H]⁺ 491.2. ¹H NMR (CD₃OD, 400 MHz): δ 7.98 (d, 1H, J=10.3 Hz), 7.78 (d, 1H, J=9.3 Hz), 7.61 (dd, 1H, J=5.4, 8.8 Hz), 7.55 (dd, 1H, J=2.9, 8.8 Hz), 7.4-7.5 (m, 1H), 6.67 (d, 1H, J=9.8 Hz), 6.50 (d, 1H, J=5.4 Hz), 5.76 (d, 1H, J=10.8 Hz), 4.98 (d, 1H, J=10.8 Hz), 4.24 (q, 2H, J=14.5 Hz), 3.1-3.3 (m, 3H), 2.8-2.9 (m, 1H), 2.6-2.8 (m, 1H), 2.31 (ddd, 1H, J=7.8, 9.0, 14.9 Hz), 1.8-2.0 (m, 1H).

### Example 108

### 2⁶,3⁴-Difluoro-2⁷-(trifluoromethyl)-1¹ 1²,2¹,2²,2³ 2⁴-hexahydro-5-aza-2(3,1)-quinazolina-1(5,6)-pyridina-3(1,2)-benzenacyclooctaphane-1²,2⁴-dione hydrochloride

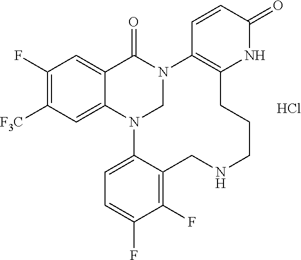 [SMILES: O=C1c2cc(F)c(C(F)(F)F)cc2N2CN1c1ccc(=O)[nH]c1CCCNCc1c2ccc(F)c1F ; Cl]

### Step A: Methyl 2-((3,4-difluoro-2-formylphenyl)amino)-5-fluoro-4-(trifluoromethyl)-benzoate 108a

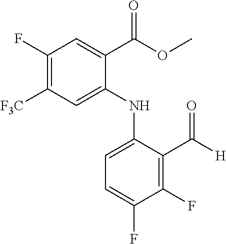 [SMILES: COC(=O)c1cc(F)c(C(F)(F)F)cc1Nc1ccc(F)c(F)c1C=O]

Following the procedure outlined in Example 44, Step A, substituting 2-bromo-5-fluorobenzaldehyde with 6-bromo-2,3-difluorobenzaldehyde and heating to 80° C. for 20 h, methyl 2-((3,4-difluoro-2-formylphenyl)amino)-5-fluoro-4-(trifluoromethyl)benzoate 108a (2.26 g, 64% yield) was prepared as a yellow solid. HPLC/MS 1.29 min (A), [M+H]⁺ 378.0. ¹H NMR (CDCl₃, 400 MHz): δ 11.29 (s, 1H), 10.45 (s, 1H), 7.7-7.9 (m, 2H), 7.3-7.4 (m, 1H), 7.1-7.1 (m, 1H), 4.03 (s, 3H).

### Step B: Methyl 2-((2-(((tert-butoxycarbonyl)(3-(6-methoxy-3-nitropyridin-2-yl)propyl)-amino)methyl)-3,4-difluorophenyl)amino)-5-fluoro-4-(trifluoromethyl)benzoate 108b

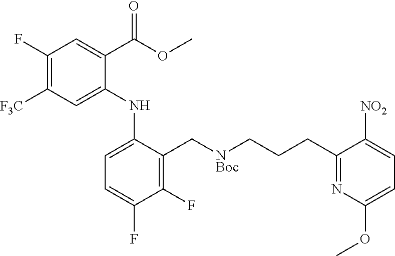 [SMILES: COC(=O)c1cc(F)c(C(F)(F)F)cc1Nc1ccc(F)c(F)c1CN(CCCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with 108a and 2-(6-methoxy-3-nitropyridin-2-yl, ethan-1-amine with Int-1h, methyl 2-((2-(((tert-butoxycarbonyl)(3-(6-methoxy-3-nitropyridin-2-yl)propyl)amino)methyl)-3,4-difluorophenyl)amino)-5-fluoro-4-(trifluoro-methyl)benzoate 108b (2.43 g, 85% yield) was prepared as a yellow foam. HPLC/MS 1.59 min (A), [M+H]⁺ 673.9. ¹H NMR (CDCl₃, 400 MHz): δ 9.15 (br s, 1H), 8.26 (d, 1H, J=8.8 Hz), 7.7-7.9 (m, 1H), 7.1-7.2 (m, 1H), 7.0-7.1 (m, 1H), 6.9-7.0 (m, 1H), 6.70 (d, 1H, J=9.3 Hz), 4.60 (d, 2H, J=1.5 Hz), 4.01 (s, 3H), 3.96 (s, 3H), 3.32 (s, 2H), 3.06 (br d, 2H, J=7.8 Hz), 1.9-2.1 (m, 2H), 1.41 (s, 10H).

### Step C: 2-((2-(((tert-Butoxycarbonyl)(3-(6-methoxy-3-nitropyridin-2-yl)propyl)amino)-methyl)-3,4-difluorophenyl)amino)-5-fluoro-4-(trifluoromethyl)benzoic acid 108c

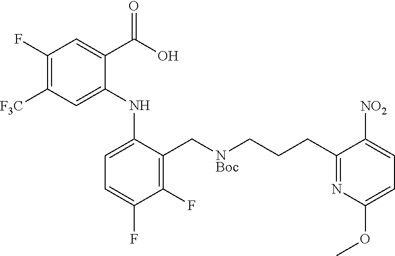 [SMILES: COc1ccc([N+](=O)[O-])c(CCCN(Cc2c(Nc3cc(C(F)(F)F)c(F)cc3C(=O)O)ccc(F)c2F)C(=O)OC(C)(C)C)n1]

Following the procedure outlined in Example 27, Step B, stirring the reaction mixture at 50° C. for 3 h, 2-((2-(((tert-butoxycarbonyl)(3-(6-methoxy-3-nitropyridin-2-yl)propyl)amino)-methyl)-3,4-difluorophenyl)amino)-5-fluoro-4-(trifluoromethyl)benzoic acid 108c (2.33 g, 95% yield) was prepared as a yellow foam. HPLC/MS 1.50 min (A), [M+H]⁺ 659.2. ¹H NMR (DMSO-d₆, 400 MHz): δ 13.3-14.1 (m, 1H), 9.27 (s, 1H), 8.33 (d, 1H, J=8.8 Hz), 7.78 (d, 1H, J=10.8 Hz), 7.44 (s, 1H), 7.1-7.3 (m, 1H), 6.87 (d, 1H, J=8.8 Hz), 6.73 (d, 1H, J=5.9 Hz), 4.50 (s, 2H), 3.90 (s, 3H), 3.14 (br t, 2H, J=7.3 Hz), 2.87 (t, 2H, J=7.6 Hz), 1.7-1.9 (m, 2H), 1.2-1.3 (m, 10H).

### Step D: 2-((2-(((3-(3-Amino-6-methoxypyridin-2-yl)propyl)(tert-butoxycarbonyl)-amino)methyl)-3,4-fluorophenyl)amino)-5-fluoro-4-(trifluoromethyl)benzoic acid 108d

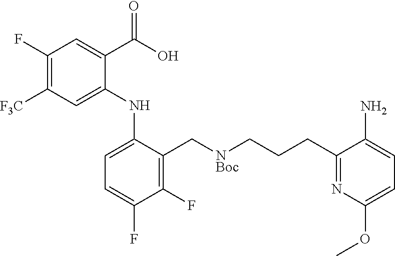 [SMILES: COc1ccc(N)c(CCCN(Cc2c(Nc3cc(C(F)(F)F)c(F)cc3C(=O)O)ccc(F)c2F)C(=O)OC(C)(C)C)n1]

Following the procedure outlined in Example 44, Step D, 2-((2-(((3-(3-amino-6-methoxypyridin-2-yl)propyl)(tert-butoxycarbonyl)amino)methyl)-4,5-difluorophenyl)amino)-5-fluoro-4-(trifluoromethyl)benzoic acid 108d (1.91 g, 51% yield) was prepared as a light brown foam. HPLC/MS 1.11 min (A), [M+H]⁺ 629.3.

### Step E: tert-Butyl 2,8,9-trifluoro-16-methoxy-20-oxo-3-(trifluoromethyl)-5,12,13,14,19,20-hexahydrodibenzo[c,f]pyrido[3,2-j][1,5,9]triazacyclotetradecine-11(10H)-carboxylate 108e

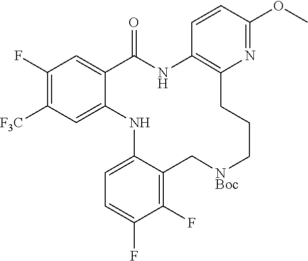 [SMILES: COc1ccc2c(n1)CCCN(C(=O)OC(C)(C)C)Cc1c(ccc(F)c1F)Nc1cc(C(F)(F)F)c(F)cc1C(=O)N2]

Following the procedure outlined in Example 27, Step D, stirring the reaction mixture at room temperature for 20 h, tert-butyl 2,8,9-trifluoro-16-methoxy-20-oxo-3-(trifluoromethyl)-5,12,13,14,19,20-hexahydrodibenzo[c,f]pyrido[3,2-j][1,5,9]triazacyclotetradecine-11(10H)-carboxylate 108e (0.566 g, 79% yield) was prepared as a white foam. HPLC/MS 1.43 min (A), [M+H]⁺ 611.2. ¹H NMR (CDCl₃, 400 MHz): δ 10.3-10.5 (m, 1H), 8.5-8.6 (m, 1H), 8.2-8.4 (m, 1H), 7.6-7.7 (m, 1H), 7.57 (d, 1H, J=6.4 Hz), 6.93 (d, 1H, J=9.3 Hz), 6.64 (d, 1H, J=9.3 Hz), 6.1-6.2 (m, 1H), 4.9-5.0 (m, 2H), 3.89 (s, 3H), 3.59 (br s, 2H), 2.4-2.6 (m, 2H), 1.46 (s, 11H).

### Step F: tert-Butyl 2⁶,3³,3⁴-trifluoro-1⁶-methoxy-2⁴-oxo-2⁷-(trifluoromethyl)-2¹,2²,2³ 2⁴-tetrahydro-5-aza-2(3,1)-quinazolina-1(3,2)-pyridina-3(1,2)-benzenacyclooctaphane-5-carboxylate 108f

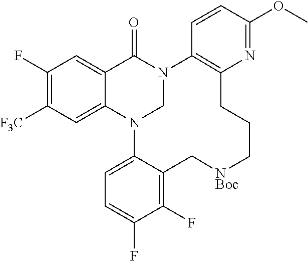 [SMILES: COc1ccc2c(n1)CCCN(C(=O)OC(C)(C)C)Cc1c(ccc(F)c1F)N1CN2C(=O)c2cc(F)c(C(F)(F)F)cc21]

Following the procedure outlined in Example 27, Step E, stirring the reaction mixture at 90° C. for 24 h, tert-butyl 2⁶,3³,3⁴-trifluoro-16-methoxy-24-oxo-2⁷-(trifluoromethyl)-2¹,2²,2³2⁴-tetrahydro-5-aza-2(3,1)-quinazolina-1(3,2)-pyridina-3(1,2)-benzenacyclooctaphane-5-carboxylate 108f (280 mg, 83% yield) was prepared as a white solid. HPLC/MS 1.42 min (A), [M+H]⁺ 623.2. ¹H NMR (CDCl₃, 400 MHz): δ 7.9-8.1 (m, 1H), 7.43 (d, 1H, J=8.3 Hz), 7.2-7.3 (m, 1H), 7.0-7.1 (m, 1H), 6.69 (d, 1H, J=8.8 Hz), 6.5-6.6 (m, 1H), 5.7-5.8 (m, 1H), 5.0-5.3 (m, 1H), 4.69 (d, 1H, J=10.8 Hz), 4.2-4.3 (m, 1H), 3.95 (s, 3H), 3.6-3.7 (m, 1H), 2.8-3.0 (m, 1H), 2.6-2.7 (m, 1H), 2.4-2.5 (m, 1H), 2.09 (s, 2H), 1.8-1.9 (m, 1H), 1.51 (s, 9H).

### Step G: 2⁶,3³,3⁴-Trifluoro-2⁷-(trifluoromethyl)-1¹²,21,22′,2³ 2⁴-hexahydro-5-aza-2(3,1)-quinazolina-1(5,6)-pyridina-3(1,2)-benzenacyclooctaphane-1²,2⁴-dione hydrochloride

### Example 108

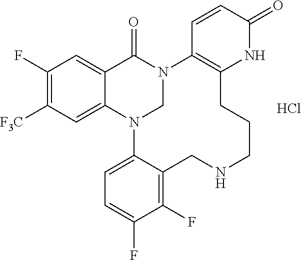 [SMILES: O=C1c2cc(F)c(C(F)(F)F)cc2N2CN1c1ccc(=O)[nH]c1CCCNCc1c2ccc(F)c1F ; Cl]

Following the procedure outlined in Example 27, Step F, stirring the reaction mixture at 90° C. for 72 h, 2⁶,3³,3⁴-trifluoro-2⁷-(trifluoromethyl)-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride (198 mg, 80% yield) was prepared. HPLC/MS 0.66 min (A), [M+H]⁺ 509.2. ¹H NMR (CD₃OD, 400 MHz): δ 7.9-8.1 (m, 2H), 7.69 (d, 1H, J=9.8 Hz), 7.44 (ddd, 1H, J=2.0, 4.2, 9.0 Hz), 6.85 (d, 1H, J=9.3 Hz), 6.53 (d, 1H, J=5.4 Hz), 5.78 (d, 1H, J=10.8 Hz), 5.03 (d, 1H, J=10.8 Hz), 4.53 (d, 1H, J=14.2 Hz), 4.13 (br d, 1H, J=14.7 Hz), 3.1-3.3 (m, 2H), 2.7-3.0 (m, 2H), 2.2-2.5 (m, 1H), 1.9-2.1 (m, 1H).

### Example 109

### 3³-Chloro-2⁶,2⁷,3⁴-trifluoro-1¹,1²,2¹,2²,2³ 2⁴-hexahydro-5-aza-2(3,1)-quinazolina-1(5,6)-pyridina-3(1,2)-benzenacyclooctaphane-1²,2⁴-dione hydrochloride

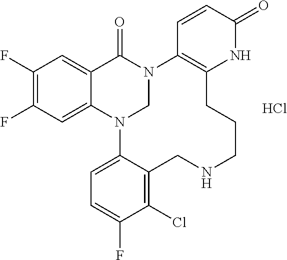 [SMILES: O=C1c2cc(F)c(F)cc2N2CN1c1ccc(=O)[nH]c1CCCNCc1c2ccc(F)c1Cl ; Cl]

### Step A: Methyl 2-((3-chloro-4-fluoro-2-formylphenyl)amino)-4,5-difluorobenzoate 109a

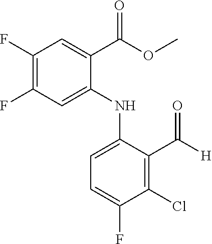 [SMILES: COC(=O)c1cc(F)c(F)cc1Nc1ccc(F)c(Cl)c1C=O]

Following the procedure outlined in Example 44, Step A, substituting 2-bromo-5-fluorobenzaldehyde with 6-bromo-2-chloro-3-fluorobenzaldehyde, methyl 2-amino-5-fluoro-4-(trifluoromethyl)benzoate with methyl 2-amino-4,5-difluorobenzoate, and heating to 80° C. for 2 h, methyl 2-((3-chloro-4-fluoro-2-formylphenyl)amino)-4,5-difluorobenzoate 109a (5.86 g, 66% yield) was prepared as a yellow solid. HPLC/MS 1.26 min (A), [M+H]⁺ 344. ¹H NMR (DMSO-d₆, 400 MHz): δ 11.15 (s, 1H), 10.42 (s, 1H), 7.93 (dd, 1H, J=9.3, 11.2 Hz), 7.5-7.7 (m, 2H), 7.50 (dd, 1H, J=4.2, 9.5 Hz), 3.88 (s, 3H).

### Step B: Methyl 2-((2-(((tert-butoxycarbonyl)(3-(6-methoxy-3-nitropyridin-2-yl)propyl)-amino)methyl)-3-chloro-4-fluorophenyl)amino)-4,5-difluorobenzoate 109b

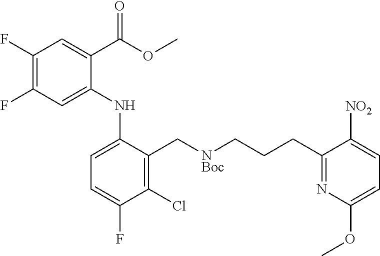 [SMILES: COC(=O)c1cc(F)c(F)cc1Nc1ccc(F)c(Cl)c1CN(CCCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with 109a and 2-(6-methoxy-3-nitropyridin-2-yl, ethan-1-amine with t-ihi, methyl 2-((2-(((tert-butoxycarbonyl)(3-(6-methoxy-3-nitropyridin-2-yl)propyl)amino)methyl)-3-chloro-4-fluorophenyl)amino)-4,5-difluorobenzoate 109b (4.91 g, 83% yield) was prepared. HPLC/MS 1.67 min (B), [M+H]⁺ 639. ¹H NMR (DMSO-d₆, 400 MHz): δ 9.10 (s, 1H), 8.32 (d, 1H, J=8.8 Hz), 7.77 (dd, 1H, J=9.0, 11.5 Hz), 7.4-7.5 (m, 1H), 7.3-7.4 (m, 1H), 6.87 (d, 1H, J=8.8 Hz), 6.52 (br dd, 1H, J=6.8, 13.2 Hz), 4.58 (s, 2H), 3.89 (s, 3H), 3.82 (s, 3H), 3.09 (br t, 2H, J=7.3 Hz), 2.85 (t, 2H, J=7.6 Hz), 1.7-1.9 (m, 2H), 1.28 (s, 9H).

### Step C: 2-((2-(((tert-Butoxycarbonyl)(3-(6-methoxy-3-nitropyridin-2-yl)propyl)amino)-methyl)-3-chloro-4-fluorophenyl)amino)-4,5-difluorobenzoic acid 109c

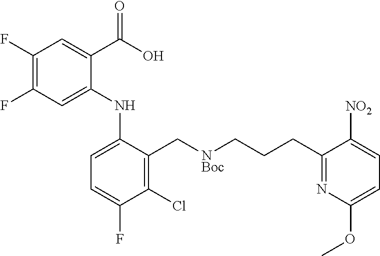 [SMILES: COc1ccc([N+](=O)[O-])c(CCCN(Cc2c(Nc3cc(F)c(F)cc3C(=O)O)ccc(F)c2Cl)C(=O)OC(C)(C)C)n1]

Following the procedure outlined in Example 27, Step B, stirring the reaction mixture at RT for 1.5 h, 2-((2-(((tert-butoxycarbonyl)(3-(6-methoxy-3-nitropyridin-2-yl)propyl)amino)-methyl)-3-chloro-4-fluorophenyl)amino)-4,5-difluorobenzoic acid 109c (4.91 g, 99% yield) was prepared. HPLC/MS 1.13 min (B), [M+H]⁺ 625. ¹H NMR (DMSO-d₆, 400 MHz): δ 9.41 (br s, 1H), 8.31 (d, 1H, J=8.8 Hz), 7.74 (dd, 1H, J=9.3, 11.7 Hz), 7.3-7.5 (m, 2H), 6.86 (d, 1H, J=9.3 Hz), 6.51 (dd, 1H, J=7.1, 13.4 Hz), 4.58 (s, 2H), 3.89 (s, 3H), 3.09 (br t, 2H, J=7.3 Hz), 2.83 (t, 2H, J=7.6 Hz), 1.7-1.8 (m, 2H), 1.28 (s, 9H).

### Step D: 2-((2-(((3-(3-Amino-6-methoxypyridin-2-yl)propyl)(tert-butoxycarbonyl)-amino)methyl)-3-chloro-4-fluorophenyl)amino)-4,5-difluorobenzoic acid 109d

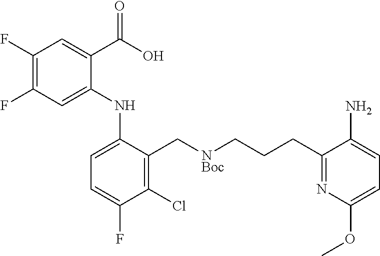 [SMILES: COc1ccc(N)c(CCCN(Cc2c(Nc3cc(F)c(F)cc3C(=O)O)ccc(F)c2Cl)C(=O)OC(C)(C)C)n1]

Following the procedure outlined in Example 44, Step D, 2-((2-(((3-(3-amino-6-methoxypyridin-2-yl)propyl)(tert-butoxycarbonyl)amino)methyl)-3-chloro-4-fluorophenyl)-amino)-4,5-difluorobenzoic acid 109d (4.57 g, 100% yield) was prepared. HPLC/MS 1.02 min (B), [M+H]⁺ 595. ¹H NMR (DMSO-d₆, 400 MHz): δ 9.47 (br s, 1H), 7.78 (dd, 1H, J=9.3, 11.2 Hz), 7.4-7.5 (m, 1H), 7.3-7.4 (m, 1H), 6.96 (d, 1H, J=8.3 Hz), 6.51 (dd, 1H, J=6.8, 13.2 Hz), 6.37 (d, 1H, J=8.3 Hz), 4.55 (s, 2H), 3.64 (s, 3H), 3.1-3.1 (m, 2H), 2.36 (t, 2H, J=7.1 Hz), 1.7-1.8 (m, 2H), 1.25 (s, 9H).

### Step E: tert-Butyl 9-chloro-2,3,8-trifluoro-16-methoxy-20-oxo-5,12,13,14,19,20-hexa-hydrodibenzo[c,f]pyrido[3,2-j][1,5,9]triazacyclotetradecine-11(10H)-carboxylate 109e

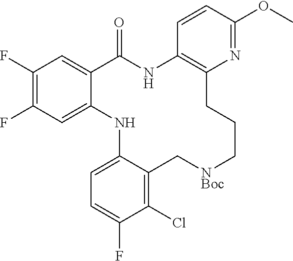 [SMILES: COc1ccc2c(n1)CCCN(C(=O)OC(C)(C)C)Cc1c(ccc(F)c1Cl)Nc1cc(F)c(F)cc1C(=O)N2]

Following the procedure outlined in Example 27, Step D, stirring the reaction mixture at RT for 20 h, tert-butyl 9-chloro-2,3,8-trifluoro-16-methoxy-20-oxo-5,12,13,14,19,20-hexahydrodibenzo[c,f]pyrido[3,2-j][1,5,9]triazacyclotetradecine-11(10H)-carboxylate 109e (2.97 g, 66% yield) was prepared. HPLC/MS 1.52 min (B), [M+H]⁺ 577. ¹H NMR (DMSO-d₆, 400 MHz): δ 10.04 (s, 1H), 7.7-7.8 (m, 2H), 7.48 (s, 1H), 7.36 (t, 1H, J=9.0 Hz), 7.13 (dd, 1H, J=4.9, 8.8 Hz), 6.71 (d, 2H, J=8.8 Hz), 4.53 (s, 2H), 3.82 (s, 3H), 2.9-3.0 (m, 2H), 2.5-2.6 (m, 2H), 1.6-1.8 (m, 2H), 1.42 (s, 9H).

### Step F: tert-Butyl 3³-chloro-2⁶,2⁷,3⁴-trifluoro-1⁶-methoxy-24-oxo-2¹,2²,2³,2⁴-tetrahydro-5-aza-2(3,1)-quinazolina-1(3,2)-pyridina-3(1,2)-benzenacyclooctaphane-5-carboxylate 109f

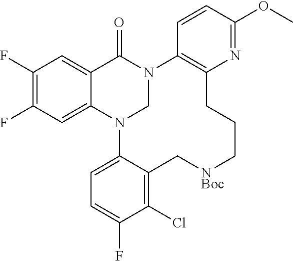 [SMILES: COc1ccc2c(n1)CCCN(C(=O)OC(C)(C)C)Cc1c(ccc(F)c1Cl)N1CN2C(=O)c2cc(F)c(F)cc21]

Following the procedure outlined in Example 27, Step E, stirring the reaction mixture at 80° C. for 18 h, tert-butyl 3³-chloro-2⁶,2⁷,3⁴-trifluoro-1⁶-methoxy-24-oxo-2¹,2²,2³,2⁴-tetrahydro-5-aza-2(3,1)-quinazolina-1(3,2)-pyridina-3(1,2)-benzenacyclooctaphane-5-carboxylate 109f (2.38 g, 79% yield) was prepared. HPLC/MS 1.41 min (B), [M+H]⁺ 589. ¹H NMR (DMSO-d₆, 400 MHz): δ 7.85 (dd, 1H, J=8.8, 10.8 Hz), 7.68 (d, 1H, J=8.8 Hz), 7.5-7.6 (m, 1H), 7.4-7.5 (m, 1H), 6.75 (d, 1H, J=8.3 Hz), 6.55 (dd, 1H, J=6.6, 12.0 Hz), 5.71 (d, 1H, J=11.2 Hz), 4.85 (d, 2H, J=11.2 Hz), 4.24 (br d, 1H, J=14.7 Hz), 3.84 (s, 3H), 3.4-3.5 (m, 1H), 2.73 (td, 1H, J=7.0, 13.8 Hz), 2.3-2.5 (m, 2H), 1.8-2.0 (m, 1H), 1.55 (br t, 1H, J=6.1 Hz), 1.39 (s, 9H).

### Step G: 3³-Chloro-2⁶,2⁷,3⁴-trifluoro-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride

### Example 109

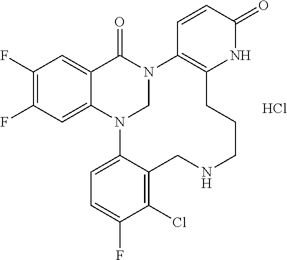 [SMILES: O=C1c2cc(F)c(F)cc2N2CN1c1ccc(=O)[nH]c1CCCNCc1c2ccc(F)c1Cl ; Cl]

Following the procedure outlined in Example 27, Step F, stirring the reaction mixture at 85° C. for 4 days, 3³-chloro-2⁶,2⁷,3⁴-trifluoro-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride (453 mg, 98% yield) was prepared. HPLC/MS 0.58 min (B), [M+H]⁺ 475. ¹H NMR (DMSO-d₆, 400 MHz): δ 9.99 (br d, 1H, J=2.9 Hz), 8.8-8.9 (m, 1H), 7.87 (dd, 1H, J=8.8, 10.3 Hz), 7.7-7.8 (m, 1H), 7.52 (dd, 1H, J=4.9, 8.8 Hz), 7.44 (d, 1H, J=9.3 Hz), 6.41 (dd, 1H, J=6.6, 12.0 Hz), 6.29 (d, 1H, J=9.3 Hz), 5.67 (d, 1H, J=10.3 Hz), 4.74 (d, 1H, J=10.8 Hz), 4.3-4.4 (m, 1H), 3.0-3.1 (m, 2H), 2.5-2.6 (m, 3H), 2.0-2.2 (m, 1H), 1.7-1.9 (m, 1H).

### Example 110

### Step A: 3³-Chloro-2⁶,3⁴-difluoro-1²,2⁴-dioxo-1¹,1²,2¹,2²,2³ 2⁴-hexahydro-5-aza-2(3,1)-quinazolina-1(5,6)-pyridina-3(1,2)-benzenacyclooctaphane-2⁷-carbonitrile hydrochloride

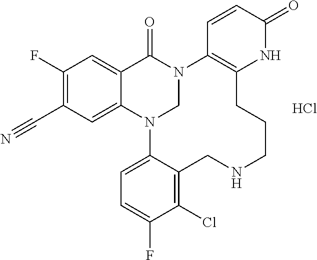 [SMILES: N#Cc1cc2c(cc1F)C(=O)N1CN2c2ccc(F)c(Cl)c2CNCCCc2[nH]c(=O)ccc21 ; Cl]

Following the procedure outlined in Example 55, Step A, with Example 109 as substrate and stirring at 60° C. for 18 h, followed by addition of additional cyanation reagents and heating for 24 h, neutral desired product was prepared, which was dissolved in 4M HCl in dioxane and concentrated to afford 3³-chloro-2⁶,3⁴-difluoro-1²,2⁴-dioxo-1¹,1²,2¹,2²,2³,2⁴-hexahydro-5-aza-2(3,1)-quinazolina-1(5,6)-pyridina-3(1,2)-benzenacyclo-octaphane-2⁷-carbonitrile hydrochloride (48.9 mg, 10% yield). HPLC/MS 0.88 min (B), [M+H]⁺ 482. ¹H NMR (DMSO-d₆, 400 MHz): δ 11.69 (br d, 1H, J=2.4 Hz), 7.84 (d, 1H, J=9.3 Hz), 7.5-7.6 (m, 1H), 7.4-7.5 (m, 1H), 7.39 (d, 1H, J=9.3 Hz), 6.86 (d, 1H, J=4.9 Hz), 6.2-6.2 (m, 1H), 5.51 (br d, 1H, J=9.8 Hz), 4.89 (br d, 1H, J=10.3 Hz), 3.8-3.9 (m, 1H), 3.6-3.7 (m, 1H), 2.5-2.7 (m, 4H), 2.1-2.2 (m, 1H), 1.9-2.0 (m, 1H), 1.5-1.6 (m, 1H).

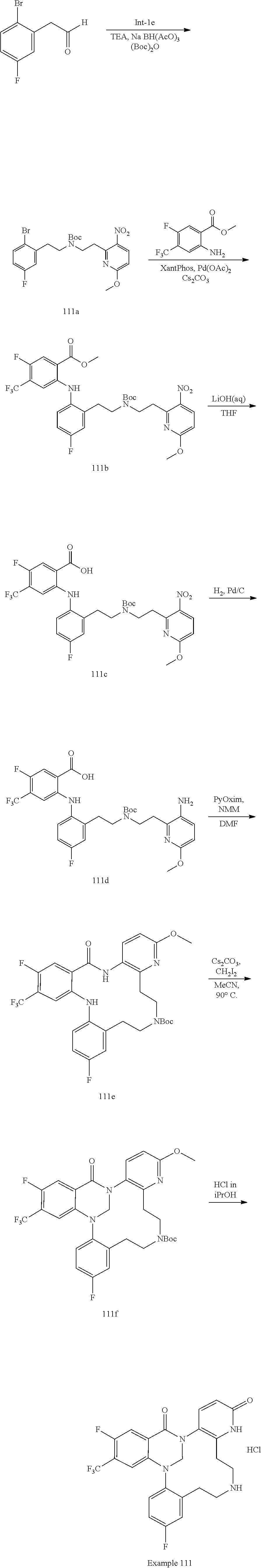

### Example 111

### 2⁶,3⁴-Difluoro-2⁷-(trifluoromethyl)-1¹,1²,2¹,2²,2³,2⁴-hexahydro-6-aza-2(3,1)-quinazolina-1(5,6)-pyridina-3(1,2)-benzenacyclooctaphane-1²,2⁴-dione hydrochloride

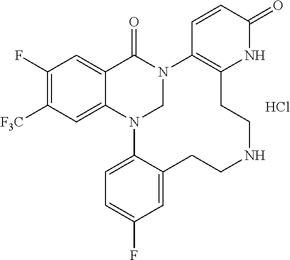 [SMILES: O=C1c2cc(F)c(C(F)(F)F)cc2N2CN1c1ccc(=O)[nH]c1CCNCCc1cc(F)ccc12 ; Cl]

### Step A: tert-Butyl (2-bromo-5-fluorophenethyl)(2-(6-methoxy-3-nitropyridin-2-yl)ethyl)-carbamate 111a

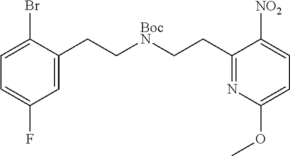 [SMILES: COc1ccc([N+](=O)[O-])c(CCN(CCc2cc(F)ccc2Br)C(=O)OC(C)(C)C)n1]

Following the preparation outlined in Example 44, Step B, substituting methyl 5-fluoro-2-((4-fluoro-2-formylphenyl)amino)-4-(trifluoromethyl)benzoate with 2-(2-bromo-5-fluorophenyl) acetaldehyde, tert-butyl (2-bromo-5-fluorophenethyl)(2-(6-methoxy-3-nitropyridin-2-yl)ethyl)-carbamate 111a (5.60 g, 50% yield) was prepared as a light yellow oil. HPLC/MS 1.52 min (B), [M+H]⁺ 498.1, 500.1. ¹H NMR (DMSO-d₆, 400 MHz): δ 8.3-8.4 (m, 1H), 7.61 (dd, 1H, J=5.6, 8.6 Hz), 7.1-7.2 (m, 1H), 7.0-7.1 (m, 1H), 6.8-6.9 (m, 1H), 3.9-4.0 (m, 3H), 3.5-3.7 (m, 2H), 3.3-3.4 (m, 2H), 3.1-3.3 (m, 2H), 2.91 (br d, 2H, J=6.8 Hz), 1.2-1.2 (m, 9H).

### Step B: Methyl 2-((2-(2-((tert-butoxycarbonyl)(2-(6-methoxy-3-nitropyridin-2-yl)ethyl)-amino)ethyl)-4-fluorophenyl)amino)-5-fluoro-4-(trifluoromethyl)benzoate 111b

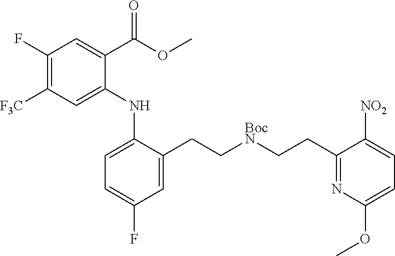 [SMILES: COC(=O)c1cc(F)c(C(F)(F)F)cc1Nc1ccc(F)cc1CCN(CCc1nc(OC)ccc1[N+](=O)[O-])C(=O)OC(C)(C)C]

Following the preparation outlined in Example 7, Step A, substituting ethyl 2-bromo-5-(trifluoromethyl)benzoate with Int-2j, Int-3a with tert-butyl (2-bromo-5-fluorophenethyl)(2-(6-methoxy-3-nitropyridin-2-yl)ethyl)carbamate 111a, BINAP with Xantphos, toluene with 1,4-dioxane, and stirring the reaction mixture at 90° C. for 32 h, methyl 2-((2-(2-((tert-butoxycarbonyl)(2-(6-methoxy-3-nitropyridin-2-yl)ethyl)amino)ethyl)-4-fluorophenyl)amino)-5-fluoro-4-(trifluoro-methyl)benzoate 111b (4.98 g, 70% yield) was prepared as a yellow solid. HPLC/MS 1.68 min (B), [M+H]⁺ 655.3. ¹H NMR (DMSO-d₆, 400 MHz): δ 9.03 (s, 1H), 8.3-8.4 (m, 1H), 7.80 (d, 1H, J=11.2 Hz), 7.33 (br s, 1H), 7.1-7.2 (m, 2H), 6.7-6.9 (m, 2H), 3.8-3.9 (m, 6H), 3.51 (br t, 2H, J=6.6 Hz), 3.3-3.4 (m, 2H), 3.0-3.2 (m, 2H), 2.7-2.8 (m, 2H), 1.0-1.1 (m, 9H).

### Step C: 2-((2-(2-((tert-Butoxycarbonyl)(2-(6-methoxy-3-nitropyridin-2-yl)ethyl)amino)-ethyl)-4-fluorophenyl)amino)-5-fluoro-4-(trifluoromethyl)benzoic acid 111c

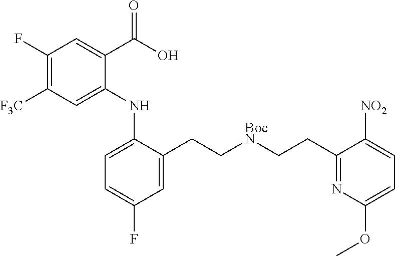 [SMILES: COc1ccc([N+](=O)[O-])c(CCN(CCc2cc(F)ccc2Nc2cc(C(F)(F)F)c(F)cc2C(=O)O)C(=O)OC(C)(C)C)n1]

Following the procedure outlined in Example 27, Step B, stirring the reaction mixture at 50° C. for 5 h, 2-((2-(2-((tert-butoxycarbonyl)(2-(6-methoxy-3-nitropyridin-2-yl)ethyl)amino)-ethyl)-4-fluorophenyl)amino)-5-fluoro-4-(trifluoromethyl)benzoic acid 111c (5.02 g, 100% yield) was prepared as a yellow solid. HPLC/MS 1.15 min (B), [M+H]⁺ 641.1. ¹H NMR (DMSO-d₆, 400 MHz): δ 13.4-14.0 (m, 1H), 9.34 (br s, 1H), 8.32 (br d, 1H, J=8.3 Hz), 7.7-7.8 (m, 1H), 7.3-7.4 (m, 1H), 7.1-7.2 (m, 2H), 6.7-6.9 (m, 2H), 3.8-3.9 (m, 3H), 3.5-3.6 (m, 2H), 3.2-3.4 (m, 2H), 3.0-3.2 (m, 2H), 2.7-2.8 (m, 2H), 1.1-1.1 (m, 9H).

### Step D: 2-((2-(2-((2-(3-Amino-6-methoxypyridin-2-yl)ethyl)(tert-butoxycarbonyl)-amino)ethyl)-4-fluorophenyl)amino)-5-fluoro-4-(trifluoromethyl)benzoic acid 111d

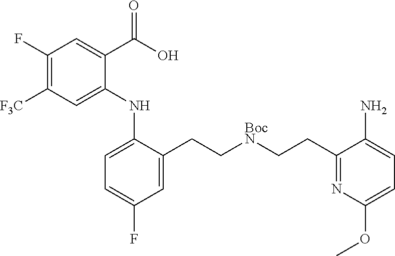 [SMILES: COc1ccc(N)c(CCN(CCc2cc(F)ccc2Nc2cc(C(F)(F)F)c(F)cc2C(=O)O)C(=O)OC(C)(C)C)n1]

Following the procedure outlined in Example 44, Step D, 2-((2-(2-((2-(3-amino-6-methoxypyridin-2-yl)ethyl)(tert-butoxycarbonyl)amino)ethyl)-4-fluorophenyl)amino)-5-fluoro-4-(trifluoromethyl)benzoic acid 111d (4.53 g, 93% yield) was prepared as a tan solid. HPLC/MS 1.06 min (B), [M+H]⁺ 611.2. ¹H NMR (DMSO-d₆, 400 MHz): δ 9.4-9.6 (m, 1H), 7.8-7.8 (m, 1H), 7.3-7.4 (m, 1H), 7.16 (br d, 2H, J=9.3 Hz), 7.03 (d, 1H, J=8.3 Hz), 6.8-6.9 (m, 1H), 6.42 (d, 1H, J=8.3 Hz), 3.6-3.7 (m, 3H), 3.3-3.5 (m, 4H), 2.6-2.8 (m, 4H), 1.2-1.3 (m, 9H) (three protons obscured).

### Step E: tert-Butyl 2,8-difluoro-16-methoxy-20-oxo-3-(trifluoromethyl)-10,11,13,14,19,20-hexahydrodibenzo[b,m]pyrido[3,2-f][1,5,10]triazacyclotetradecine-12(5H)-carboxylate 111e

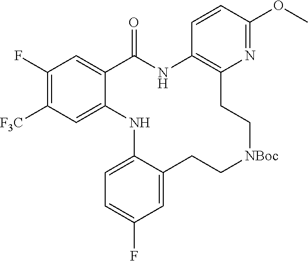 [SMILES: COc1ccc2c(n1)CCN(C(=O)OC(C)(C)C)CCc1cc(F)ccc1Nc1cc(C(F)(F)F)c(F)cc1C(=O)N2]

Following the procedure outlined in Example 27, Step D, stirring the reaction mixture at room temperature for 70 min, tert-butyl 2,8-difluoro-16-methoxy-20-oxo-3-(trifluoromethyl)-10,11,13,14,19,20-hexahydrodibenzo[b,m]pyrido[3,2-f][1,5,10]triazacyclotetradecine-12(5H)-carboxylate 111e (2.24 g, 53% yield) was prepared as a grey solid. HPLC/MS 1.56 min (B), [M+H]⁺ 593.2. ¹H NMR (DMSO-d₆, 400 MHz): δ 10.2-10.3 (m, 1H), 8.55 (s, 1H), 7.9-8.0 (m, 1H), 7.6-7.7 (m, 1H), 7.3-7.4 (m, 1H), 7.0-7.2 (m, 3H), 6.76 (dd, 1H, J=3.4, 8.8 Hz), 3.8-3.9 (m, 3H), 3.4-3.5 (m, 2H), 3.3-3.4 (m, 2H), 2.8-2.9 (m, 2H), 2.5-2.7 (m, 2H), 1.5-1.5 (m, 9H).

### Step F: tert-Butyl 2⁶,3⁴-difluoro-1⁶-methoxy-2⁴-oxo-27-(trifluoromethyl)-2¹,2²,2³ 2⁴-tetrahydro-6-aza-2(3,1)-quinazolina-1(3,2)-pyridina-3(1,2)-benzenacyclooctaphane-6-carboxylate 111f

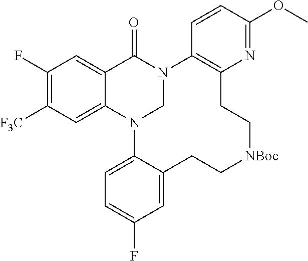 [SMILES: COc1ccc2c(n1)CCN(C(=O)OC(C)(C)C)CCc1cc(F)ccc1N1CN2C(=O)c2cc(F)c(C(F)(F)F)cc21]

Following the procedure outlined in Example 27, Step E, stirring the reaction mixture at 90° C. for 23 h, tert-butyl 2⁶,3⁴-difluoro-1⁶-methoxy-2⁴-oxo-27-(trifluoromethyl)-2¹2²,2³2⁴-tetrahydro-6-aza-2(3,1)-quinazolina-1(3,2)-pyridina-3(1,2)-benzenacyclooctaphane-6-carboxylate 111f (2.12 g, 92% yield) was prepared as a light yellow solid. HPLC/MS 1.47 min (B), [M+H]⁺ 605.3. ¹H NMR (DMSO-d₆, 400 MHz): δ 7.88 (d, 1H, J=10.8 Hz), 7.69 (br s, 1H), 7.3-7.5 (m, 2H), 7.24 (br s, 1H), 6.7-6.8 (m, 1H), 6.39 (br d, 1H, J=5.4 Hz), 5.61 (d, 1H, J=10.3 Hz), 4.55 (br d, 1H, J=18.1 Hz), 3.6-3.8 (m, 2H), 3.38 (br d, 2H, J=9.8 Hz), 3.3-3.3 (m, 3H), 3.0-3.2 (m, 2H), 2.6-2.7 (m, 2H), 1.1-1.2 (m, 9H).

### Step G: 2⁶,3⁴-Difluoro-2⁷-(trifluoromethyl)-1¹,1²,2¹,2²,2³ 2⁴-hexahydro-6-aza-2(3,1)-quinazolina-1(5,6)-pyridina-3(1,2)-benzenacyclooctaphane-1²,2⁴-dione hydrochloride

### Example 111

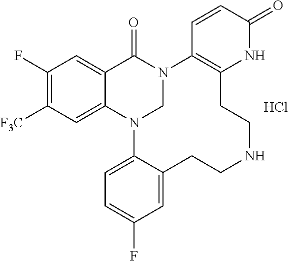 [SMILES: O=C1c2cc(F)c(C(F)(F)F)cc2N2CN1c1ccc(=O)[nH]c1CCNCCc1cc(F)ccc12 ; Cl]

Following the procedure outlined in Example 27, Step F, stirring the reaction mixture at 90° C. for 17 h, followed by additional 4N HCl-ⁱPrOH (14 equiv) and stirring at 90° C. for 94 h, 2⁶,3⁴-difluoro-2⁷-(trifluoromethyl)-1¹,1²,2¹,2²,2³,2⁴-hexahydro-6-aza-2(3,1)-quinazolina-1(5,6)-pyridina-3(1,2)-benzenacyclooctaphane-1²,2⁴-dione hydrochloride (1.72 g, 91% yield) was prepared. HPLC/MS 1.00 min (B), [M+H]⁺ 491.1. ¹H NMR (DMSO-d₆, 400 MHz): δ 9.24 (br s, 1H), 8.81 (br s, 1H), 7.94 (d, 1H, J=10.3 Hz), 7.4-7.5 (m, 2H), 7.3-7.4 (m, 2H), 6.42 (d, 1H, J=5.9 Hz), 6.36 (d, 1H, J=9.3 Hz), 5.67 (d, 1H, J=10.8 Hz), 4.8-4.9 (m, 2H), 3.2-3.4 (m, 2H), 3.0-3.1 (m, 2H), 2.7-2.9 (m, 4H).

### Example 112

### Step A: 2⁶,3⁴-Difluoro-6-methyl-2⁷-(trifluoromethyl)-1¹,1²,2¹,2²,2³,2⁴-hexahydro-6-aza-2(3,1)-quinazolina-1(5,6)-pyridina-3(1,2)-benzenacyclooctaphane-1²,2⁴-dione

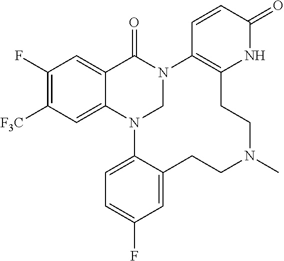 [SMILES: CN1CCc2cc(F)ccc2N2CN(C(=O)c3cc(F)c(C(F)(F)F)cc32)c2ccc(=O)[nH]c2CC1]

A mixture of 2⁶,3⁴-difluoro-2⁷-(trifluoromethyl)-1¹,1²,2¹,2²,2³,2⁴-hexahydro-6-aza-2(3,1)-quinazolina-1(5,6)-pyridina-3(1,2)-benzenacyclooctaphane-1²,2⁴-dione hydrochloride Example 111 (120 mg, 228 μmol), formic acid (2.0 mL, 53.0 mmol) and 37% aq. formaldehyde (84.8 μL, 1.14 mmol) was stirred at 80° C. for 17 h, concentrated and purified by reverse-phase semi-prep HPLC (XSELECT CSH C18 column, 150 mm×30 mm i.d. 5 μm packing diameter), eluting with 20-99% AcCN in 10 mM Ammonium Bicarbonate in H₂O (adjusted to pH 10 with Ammonia) to afford 2⁶,3⁴-difluoro-6-methyl-2⁷-(trifluoromethyl)-1¹,1²,2¹,2²,2³,2⁴-hexahydro-6-aza-2(3,1)-quinazolina-1(5,6)-pyridina-3(1,2)-benzenacyclooctaphane-1²,2⁴-dione (68 mg, 58% yield) as a white solid. HPLC/MS 1.05 min (B), [M+H]⁺ 505.1. ¹H NMR (DMSO-d₆, 400 MHz): δ 11.73 (br s, 1H), 7.86 (d, 1H, J=10.8 Hz), 7.3-7.5 (m, 3H), 7.22 (dt, 1H, J=2.9, 8.3 Hz), 6.36 (d, 1H, J=5.9 Hz), 6.23 (d, 1H, J=9.8 Hz), 5.57 (d, 1H, J=10.3 Hz), 4.78 (d, 1H, J=10.3 Hz), 2.6-2.8 (m, 3H), 2.5-2.6 (m, 3H), 2.4-2.5 (m, 2H), 2.03 (s, 3H).

### Example 113

### 3³-Chloro-2⁶,2⁷,3⁴-trifluoro-5-methyl-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride

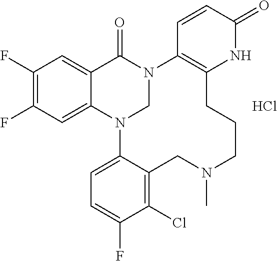 [SMILES: CN1CCCc2[nH]c(=O)ccc2N2CN(c3cc(F)c(F)cc3C2=O)c2ccc(F)c(Cl)c2C1 ; Cl]

### Step A: 3³-Chloro-2⁶,2⁷,3⁴-trifluoro-1⁶-methoxy-2¹,2²,2³ 2⁴-tetrahydro-5-aza-2(3,1)-quinazolina-1(3,2)-pyridina-3(1,2)-benzenacyclooctaphan-2⁴-one dihydrochloride 113a

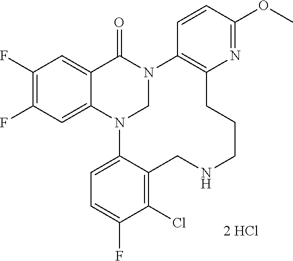 [SMILES: COc1ccc2c(n1)CCCNCc1c(ccc(F)c1Cl)N1CN2C(=O)c2cc(F)c(F)cc21 ; "2 HCl"]

To 3³-chloro-2⁶,2⁷,3⁴-trifluoro-1⁶-methoxy-2⁴-oxo-2¹,2²,2³,2⁴-tetrahydro-5-aza-2(3,1)-quinazolina-1(3,2)-pyridina-3(1,2)-benzenacyclooctaphane-5-carboxylate 109f (0.100 g, 170 μmol) in dioxane (849 μL), was added 4M HCl in dioxane (212 μL, 849 μmol). After 1 h, the reaction was concentrated to afford 3³-chloro-2⁶,2²,3⁴-trifluoro-1⁶-methoxy-2¹,2²,2³2⁴-tetrabydro-5-aza-2(3,1)-quinazolina-1(3,2)-pyridina-3(1,2)-benzenacyclooctaphan-24-one dihydrochloride 113a (0.102 g, 100% yield). HPLC/MS 0.65 min (A), [M+H]⁺ 489. ¹H NMR (DMSO-d₆, 400 MHz): δ 9.9-10.0 (m, 1H), 8.7-8.8 (m, 1H), 7.89 (dd, 1H, J=8.8, 10.8 Hz), 7.7-7.8 (m, 2H), 7.53 (dd, 1H, J=4.9, 8.8 Hz), 6.81 (d, 1H, J=8.3 Hz), 6.43 (dd, 1H, J=6.6, 12.0 Hz), 5.77 (d, 1H, J=10.8 Hz), 4.77 (d, 1H, J=10.3 Hz), 4.3-4.4 (m, 1H), 4.03 (br d, 2H, J=15.2 Hz), 3.5-3.5 (m, 1H), 3.0-3.2 (m, 2H), 2.8-2.9 (m, 1H), 2.7-2.8 (m, 1H), 2.0-2.2 (m, 1H), 1.8-1.9 (m, 1H).

### Step B: 3³-Chloro-2⁶,2⁷,3⁴-trifluoro-1⁶-methoxy-5-methyl-2¹,2²,2³ 2⁴-tetrahydro-5-aza-2(3,1)-quinazolina-1(3,2)-pyridina-3(1,2)-benzenacyclooctaphan-2⁴-one 113b

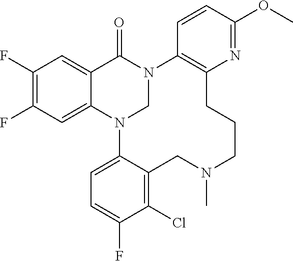 [SMILES: COc1ccc2c(n1)CCCN(C)Cc1c(ccc(F)c1Cl)N1CN2C(=O)c2cc(F)c(F)cc21]

Following the procedure outlined in Example 112, Step A, with 3³chloro-2⁶,2′ 3⁴-trifluoro-16-methoxy-21,2′,2 24-tetrahydro-5-aza-2(3,1)-quinazolina-1(3,2)-pyridina,-3(1,2)-benzenacyclooctaphan-24-one dihydrochloride 113a as the substrate, 3³-chloro-2⁶,2⁷,3⁴-trifluoro-1⁶-methoxy-5-methyl-2¹,2²,2³,2⁴-tetrahydro-5-aza-2(3,1)-quinazolina-1(3,2)-pyridina-3(1,2)-benzenacyclooctaphan-2⁴-one 113b (53.0 mg, 58% yield) was prepared. HPLC/MS 1.51 min (B), [M+H]⁺ 503. ¹H NMR (DMSO-d₆, 400 MHz): δ 7.80 (dd, 1H, J=8.8, 10.8 Hz), 7.5-7.6 (m, 3H), 6.68 (d, 1H, J=8.8 Hz), 6.40 (dd, 1H, J=6.6, 12.5 Hz), 5.41 (d, 1H, J=9.3 Hz), 4.61 (d, 1H, J=9.3 Hz), 3.89 (d, 1H, J=11.7 Hz), 3.86 (s, 3H), 3.43 (d, 1H, J=11.7 Hz), 3.0-3.1 (m, 1H), 2.4-2.5 (m, 1H), 2.3-2.4 (m, 2H), 1.8-2.1 (m, 2H), 1.70 (s, 3H).

### Step C: 33-Chloro-2⁶,2⁷,3⁴-trifluoro-5-methyl-1¹,1²,2¹,2²,2³,2⁴-hexahydro-5-aza-2(3,1)-quinazolina-1(5,6)-pyridina-3(1,2)-benzenacyclooctaphane-1²,2⁴-dione hydrochloride Example

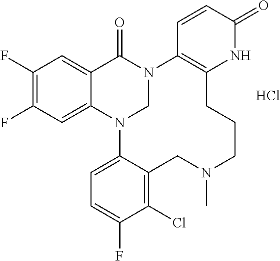 [SMILES: CN1CCCc2[nH]c(=O)ccc2N2CN(c3cc(F)c(F)cc3C2=O)c2ccc(F)c(Cl)c2C1 ; Cl]

3³-Chloro-2⁶,2⁷,3⁴-trifluoro-1⁶-methoxy-5-methyl-2¹,2²,2³,2⁴-tetrahydro-5-aza-2(3,1)-quinazolina-1(3,2)-pyridina-3(1,2)-benzenacyclooctaphan-2⁴-one 113b (53.0 mg, 105 mol) in 5M HCl in 2-propanol (0.422 mL, 2.11 mmol) was heated at 85° C. for 18 h and then concentrated under reduced pressure to afford 3³-chloro-2⁶,2⁷,3⁴-trifluoro-5-methyl-1¹,1²,2¹,2²,2³2⁴-hexahydro-5-aza-2(3,1)-quinazolina-1(5,6)-pyridina-3(1,2)-benzenacyclooctaphane-1²,2⁴-dione hydrochloride (57.0 mg, 99% yield). HPLC/MS 0.56 min (A), [M+H]⁺ 489. ¹H NMR (DMSO-d₆, 501 MHz): δ 11.13-12.52 (m, 1H), 9.98-10.39 (m, 1H), 7.88 (br d, J=10.1 Hz, 1H), 7.71-7.81 (m, 1H), 7.48 (dd, J=9.0, 5.0 Hz, 1H), 7.42 (dd, J=9.6, 2.7 Hz, 1H), 6.42-6.63 (m, 1H), 6.29 (br d, J=9.3 Hz, 1H), 5.46-5.82 (m, 1H), 4.77-5.04 (m, 1H), 4.09-4.32 (m, 2H), 3.08-3.45 (m, 2H), 3.00-3.06 (m, 3H), 1.84-2.04 (m, 2H).

### Example 114

### 8-Fluoro-10-methyl-2-(trifluoromethyl)-10,11,12,13-tetrahydro-19H-5,18-methanodibenzo[c,f]-pyrido[3,2-j][1,5,9]triazacyclotridecine-15,19(14H)-dione hydrochloride

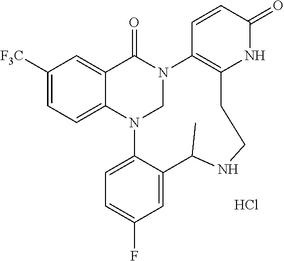 [SMILES: CC1NCCc2[nH]c(=O)ccc2N2CN(c3ccc(C(F)(F)F)cc3C2=O)c2ccc(F)cc21 ; Cl]

### Step A: Methyl 2-((2-acetyl-4-fluorophenyl)amino)-5-(trifluoromethyl)benzoate 114a

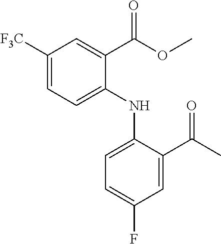 [SMILES: COC(=O)c1cc(C(F)(F)F)ccc1Nc1ccc(F)cc1C(C)=O]

Following the preparation outlined in Example 7, Step A, substituting ethyl 2-bromo-5-(trifluoromethyl)benzoate with methyl 2-bromo-5-(trifluoromethyl)benzoate, Int-3a with 1-(2-amino-5-fluorophenyl)ethan-1-one, BINAP with Xantphos, toluene with 1,4-dioxane, and stirring the reaction at 80° C. for 8 h, methyl 2-((2-acetyl-4-fluorophenyl)amino)-5-(trifluoromethyl)-benzoate 114a (6.05 g, 60% yield) was prepared as a yellow oil. HPLC/MS 1.35 min (B), [M+H]⁺ 356.0. ¹H NMR (CDCl₃, 400 MHz): δ 11.48 (s, 1H), 8.29 (d, J=2.0 Hz, 1H), 7.51-7.58 (m, 3H), 7.46 (d, J=8.8 Hz, 1H), 7.17-7.24 (m, 1H), 4.02 (s, 3H), 2.64 (s, 3H).

### Step B: Methyl 2-((2-(1-((tert-butoxycarbonyl)(2-(6-methoxy-3-nitropyridin-2-yl)ethyl)-amino)ethyl)-4-fluorophenyl)amino)-5-(trifluoromethyl)benzoate 114b

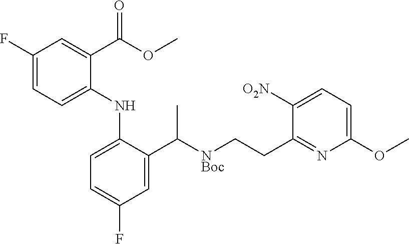 [SMILES: COC(=O)c1cc(F)ccc1Nc1ccc(F)cc1C(C)N(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((2-acetyl-4-fluorophenyl)amino)-5-(trifluoromethyl)benzoate 114a, methyl 2-((2-(1-((tert-butoxy-carbonyl)(2-(6-methoxy-3-nitropyridin-2-yl)ethyl)amino)ethyl)-4-fluorophenyl)amino)-5-(trifluoromethyl)benzoate 114b (2.44 g, 34% yield) was prepared as a clear oil. HPLC/MS 1.69 min (B), [M+H]⁺ 637.2. ¹H NMR (CDCl₃, 400 MHz): δ 9.41 (s, 1H), 8.13 (s, 1H), 8.05 (d, J=9.3 Hz, 1H), 7.34 (d, J=8.9 Hz, 1H), 7.18-7.23 (m, 2H), 6.95 (td, J=8.3, 2.9 Hz, 1H), 6.67 (d, J=8.8 Hz, 1H), 6.54 (d, J=9.3 Hz, 1H), 5.35-5.56 (m, 1H), 3.86 (d, J=4.9 Hz, 6H), 3.19-3.43 (m, 3H), 2.89 (br s, 1H), 1.46 (d, J=6.8 Hz, 3H), 1.29 (s, 9H).

### Step C: 2-((2-(1-((tert-Butoxycarbonyl)(2-(6-methoxy-3-nitropyridin-2-yl)ethyl)amino)-ethyl)-4-fluorophenyl)amino)-5-(trifluoromethyl)benzoic acid 114c

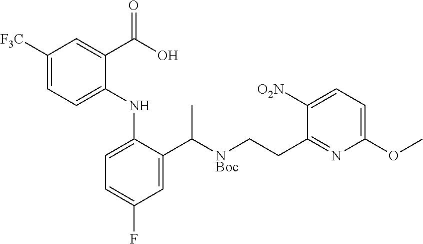 [SMILES: COc1ccc([N+](=O)[O-])c(CCN(C(=O)OC(C)(C)C)C(C)c2cc(F)ccc2Nc2ccc(C(F)(F)F)cc2C(=O)O)n1]

Following the procedure outlined in Example 41, Step C, stirring the reaction mixture at room temperature overnight, 2-((2-(1-((tert-butoxycarbonyl)(2-(6-methoxy-3-nitropyridin-2-yl)ethyl)amino)ethyl)-4-fluorophenyl)amino)-5-(trifluoromethyl)benzoic acid 114c (1.90 g, 77% yield) was prepared as a white solid. HPLC/MS 1.12 min (B), [M+H]⁺ 623.2. ¹H NMR (CDCl₃, 400 MHz): δ 9.44 (s, 1H), 8.28 (d, J=1.5 Hz, 1H), 8.18 (d, J=8.8 Hz, 1H), 7.40-7.54 (m, 1H), 7.29-7.34 (m, 1H), 7.20-7.28 (m, 1H), 7.07 (td, J=8.1, 2.9 Hz, 1H), 6.59-6.79 (m, 2H), 5.55-5.76 (m, 1H), 3.98 (s, 3H), 3.27-3.55 (m, 3H), 3.01 (ddd, J=13.2, 10.3, 5.4 Hz, 1H), 1.56 (d, J=7.3 Hz, 3H), 1.35 (s, 9H).

### Step D: 2-((2-(1-((2-(3-Amino-6-methoxypyridin-2-yl)ethyl)(tert-butoxycarbonyl-amino)ethyl)-4-fluorophenyl)amino)-5-(trifluoromethyl)benzoic acid 114d

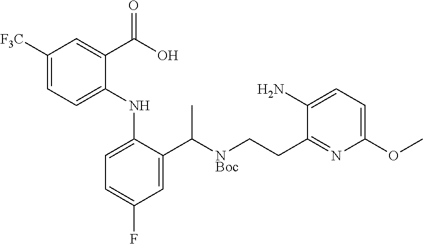 [SMILES: COc1ccc(N)c(CCN(C(=O)OC(C)(C)C)C(C)c2cc(F)ccc2Nc2ccc(C(F)(F)F)cc2C(=O)O)n1]

Following the procedure outlined in Example 44, Step D, 2-((2-(1-((2-(3-amino-6-methoxypyridin-2-yl)ethyl)(tert-butoxycarbonyl)amino)ethyl)-4-fluorophenyl)amino)-5-(trifluoromethyl)benzoic acid 114d (1.76 g, 94% yield) as a gray solid. HPLC/MS 1.05 min (B), [M+H]⁺ 593.2. ¹H NMR (CDCl₃, 400 MHz): δ 9.00 (br d, J=6.4 Hz, 1H), 8.24 (d, J=2.0 Hz, 1H), 7.37 (dd, J=8.8, 2.0 Hz, 1H), 7.17 (br d, J=7.8 Hz, 2H), 6.88-7.05 (m, 2H), 6.24-6.50 (m, 2H), 5.18 (br d, J=2.9 Hz, 1H), 3.67 (br s, 3H), 3.50 (br dd, J=9.0, 2.7 Hz, 1H), 3.11-3.37 (m, 2H), 2.59-2.73 (m, 1H), 1.29-1.43 (m, 12H).

### Step E: tert-Butyl 8-fluoro-15-methoxy-10-methyl-19-oxo-2-(trifluoromethyl)-5,10,12,13,18,19-hexahydro-11H-dibenzo[c,f]pyrido[3,2-j][1,5,9]triazacyclotridecine-11-carboxylate 114e

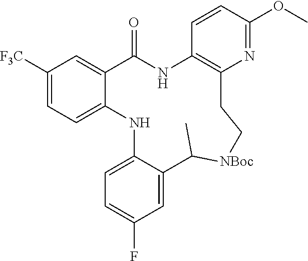 [SMILES: COc1ccc2c(n1)CCN(C(=O)OC(C)(C)C)C(C)c1cc(F)ccc1Nc1ccc(C(F)(F)F)cc1C(=O)N2]

Following the procedure outlined in Example 41, Step E, stirring the reaction mixture at room temperature for 1 h, tert-butyl 8-fluoro-15-methoxy-10-methyl-19-oxo-2-(trifluoromethyl)-5,10,12,13,18,19-hexahydro-11H-dibenzo[c,f]pyrido[3,2-j][1,5,9]triazacyclotridecine-11-carboxylate 114e (1.37 g, 77% yield) was prepared as an off-white solid. HPLC/MS 1.46 min (B), [M+H]⁺ 575.2. ¹H NMR (CDCl₃, 400 MHz): δ 10.77-11.05 (m, 1H), 8.70 (br s, 1H), 8.16 (d, J=8.8 Hz, 1H), 7.83 (dd, J=8.3, 2.0 Hz, 1H), 7.40 (d, J=8.3 Hz, 1H), 7.16 (br dd, J=9.3, 2.4 Hz, 1H), 6.79-6.90 (m, 1H), 6.61 (d, J=8.8 Hz, 1H), 6.34 (br s, 1H), 6.12-6.22 (m, 1H), 5.70-5.88 (m, 1H), 3.92 (dd, J=14.7, 11.7 Hz, 1H), 3.84 (s, 3H), 3.19 (br dd, J=14.4, 4.6 Hz, 1H), 2.28 (br dd, J=13.7, 5.4 Hz, 1H), 1.75-1.90 (m, 1H), 1.57 (d, J=6.8 Hz, 3H), 1.03 (br s, 9H).

### Step F: tert-Butyl 8-fluoro-15-methoxy-10-methyl-19-oxo-2-(trifluoromethyl)-12,13-dihydro-19H-5,18-methanodibenzo[c,f]pyrido[3,2-j][1,5,9]triazacyclotridecine-11(10H)-carboxylate 114f

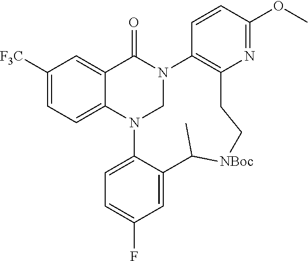 [SMILES: COc1ccc2c(n1)CCN(C(=O)OC(C)(C)C)C(C)c1cc(F)ccc1N1CN2C(=O)c2cc(C(F)(F)F)ccc21]

Following the procedure outlined in Example 41, Step F, stirring the reaction at 90° C. overnight, tert-butyl 8-fluoro-15-methoxy-10-methyl-19-oxo-2-(trifluoromethyl)-12,13-dihydro-19H-5,18-methanodibenzo[c,f]pyrido[3,2-j][1,5,9]triazacyclotridecine-11(10H)-carboxylate 114f (770 mg, 54% yield) was prepared as a light tan solid. HPLC/MS 1.47 min (B), [M+H]⁺ 587.2. ¹H NMR (CDCl₃, 400 MHz): δ 8.38 (d, J=2.4 Hz, 1H), 7.61 (dd, J=10.3, 2.9 Hz, 1H), 7.45-7.55 (m, 2H), 7.14-7.21 (m, 1H), 7.08-7.14 (m, 1H), 6.68 (d, J=8.8 Hz, 1H), 6.47 (d, J=8.3 Hz, 1H), 5.33 (d, J=10.3 Hz, 1H), 4.53 (d, J=6.8 Hz, 1H), 4.39 (d, J=10.8 Hz, 1H), 4.13 (br s, 1H), 3.96 (s, 3H), 3.33-3.44 (m, 2H), 2.65 (s, 1H), 1.62-1.67 (m, 3H), 1.13 (s, 9H).

### Step G: 8-Fluoro-10-methyl-2-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 114

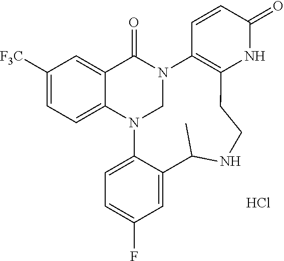 [SMILES: CC1NCCc2[nH]c(=O)ccc2N2CN(c3ccc(C(F)(F)F)cc3C2=O)c2ccc(F)cc21 ; Cl]

Following the procedure outlined in Example 41, Step G, stirring the reaction mixture at 90° C. overnight, 8-fluoro-10-methyl-2-(trifluoromethyl)-10,11,12,13-tetrahydro-19H-5,18-methano-dibenzo[c,]pyrido[3,2-j][1,5,9]triazacyclotridecine-15,19(14H)-dione hydrochloride (598 mg, 89% yield) was prepared as an off-white solid. HPLC/MS 0.98 min (B), [M+H]⁺ 473.2. ¹H NMR (DMSO-d₆, 400 MHz): δ 10.26 (br s, 1H), 9.83 (br s, 1H), 8.13 (d, J=2.0 Hz, 1H), 7.84 (dd, J=9.8, 2.9 Hz, 1H), 7.69 (dd, J=8.6, 2.2 Hz, 1H), 7.59 (dd, J=8.6, 5.6 Hz, 1H), 7.43-7.52 (m, 2H), 6.48 (d, J=8.8 Hz, 1H), 6.36 (d, J=9.3 Hz, 1H), 5.50 (d, J=11.2 Hz, 1H), 4.93 (d, J=11.2 Hz, 1H), 4.64 (br d, J=3.9 Hz, 1H), 3.20-3.35 (m, 1H), 2.90-3.03 (m, 1H), 2.79-2.89 (m, 1H), 2.67-2.77 (m, 1H), 1.54 (d, J=6.8 Hz, 3H).

### Example 115 and Example 116

### Step A: (10R)-8-Fluoro-10-methyl-2-(trifluoromethyl)-10,11,12,13-tetrahydro-19H-5,18-methanodibenzo[c,f]pyrido[3,2-j][1,5,9]triazacyclotridecine-15,19(14H)-dione (10S)-8-Fluoro-10-methyl-2-(trifluoromethyl)-10,11,12,13-tetrahydro-19H-5,18-methanodibenzo[c,f]pyrido[3,2-j][1,5,9]triazacyclotridecine-15,19(14H)-dione

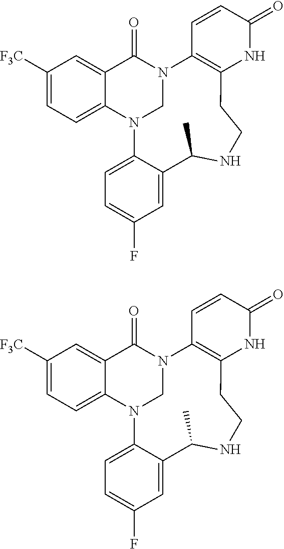 [SMILES: C[C@H]1NCCc2[nH]c(=O)ccc2N2CN(c3ccc(C(F)(F)F)cc3C2=O)c2ccc(F)cc21 ; C[C@@H]1NCCc2[nH]c(=O)ccc2N2CN(c3ccc(C(F)(F)F)cc3C2=O)c2ccc(F)cc21]

Racemic 8-fluoro-10-methyl-2-(trifluoromethyl)-10,11,12,13-tetrahydro-19H-5,18-methanodibenzo[c,]pyrido[3,2-j][1,5,9]triazacyclotridecine-15,19(14H)-dione hydrochloride Example 114 (600 mg, 1.10 mmol) was resolved using chiral chromatography (CC4 column, 150×4.6 mm, 5 μm), eluting with an isocratic gradient of methanol (0.1% isopropylamine) to afford:

First-eluting stereoisomer (10R)-8-fluoro-10-methyl-2-(trifluoromethyl)-10,11,12,13-tetrahydro-19H-5,18-methanodibenzo[c,f]pyrido[3,2-j][1,5,9]triazacyclotridecine-15,19(14H)-dione Example 115 (256 mg, 49% yield). HPLC/MS 0.98 min (B), [M+H]⁺ 473.2. ¹H NMR (CDCl₃, 400 MHz): δ 8.38 (d, J=2.0 Hz, 1H), 7.51 (dd, J=8.8, 2.0 Hz, 1H), 7.39 (d, J=9.3 Hz, 1H), 7.25-7.29 (m, 2H), 7.10 (td, J=8.1, 2.9 Hz, 1H), 6.53 (d, J=8.8 Hz, 1H), 6.47 (d, J=9.8 Hz, 1H), 5.29 (d, J=9.3 Hz, 1H), 5.07 (d, J=9.8 Hz, 1H), 4.39 (br d, J=6.8 Hz, 1H), 3.35-3.46 (m, 1H), 3.09-3.21 (m, 1H), 3.02 (br d, J=11.2 Hz, 1H), 2.53 (dt, J=13.9, 4.0 Hz, 1H), 1.43 (d, J=6.8 Hz, 3H).

Second-eluting stereoisomer (10S)-8-fluoro-10-methyl-2-(trifluoromethyl)-10,11,12,13-tetrahydro-19H-5,18-methanodibenzo[c,f]pyrido[3,2-j][1,5,9]triazacyclotridecine-15,19(14H)-dione Example 115 (251 mg, 48% yield). HPLC/MS 0.98 min (B), [M+H]⁺ 473.2. ¹H NMR (CDCl₃, 400 MHz): δ 8.38 (d, J=2.0 Hz, 1H), 7.51 (dd, J=8.8, 2.0 Hz, 1H), 7.39 (d, J=9.3 Hz, 1H), 7.25-7.29 (m, 2H), 7.10 (td, J=8.1, 2.9 Hz, 1H), 6.53 (d, J=8.8 Hz, 1H), 6.47 (d, J=9.8 Hz, 1H), 5.29 (d, J=9.3 Hz, 1H), 5.07 (d, J=9.8 Hz, 1H), 4.39 (br d, J=6.8 Hz, 1H), 3.35-3.46 (m, 1H), 3.09-3.21 (m, 1H), 3.02 (br d, J=11.2 Hz, 1H), 2.53 (dt, J=13.9, 4.0 Hz, 1H), 1.43 (d, J=6.8 Hz, 3H).

### Example 117

### 2,8-Difluoro-10-methyl-3-(trifluoromethyl)-10,11,12,13-tetrahydro-19H-5,18-methanodibenzo-[c,f]pyrido[3,2-j][1,5,9]triazacyclotridecine-15,19(14H)-dione hydrochloride

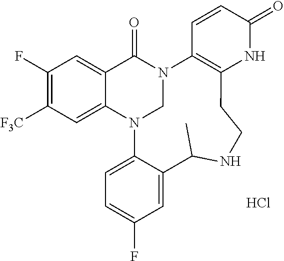 [SMILES: CC1NCCc2[nH]c(=O)ccc2N2CN(c3cc(C(F)(F)F)c(F)cc3C2=O)c2ccc(F)cc21 ; Cl]

### Step A: Methyl 2-((2-acetyl-4-fluorophenyl)amino)-5-fluoro-4-(trifluoromethyl)benzoate 117a

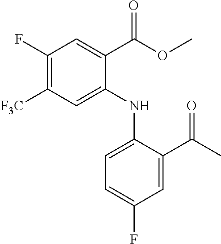 [SMILES: COC(=O)c1cc(F)c(C(F)(F)F)cc1Nc1ccc(F)cc1C(C)=O]

Following the preparation outlined in Example 7, Step A, substituting ethyl 2-bromo-5-(trifluoromethyl)benzoate with 1-(2-bromo-5-fluorophenyl)ethan-1-one, Int-3a with methyl 2-amino-5-fluoro-4-(trifluoromethyl)benzoate, BINAP with Xantphos, toluene with 1,4-dioxane, and stirring the reaction at 110° C. for 7 h, methyl 2-((2-acetyl-4-fluorophenyl)amino)-5-fluoro-4-(trifluoromethyl)benzoate 117a (670 mg, 8% yield) was prepared as a yellow solid. HPLC/MS 1.35 min (B), [M+H]⁺ 374.0. ¹H NMR (CDCl₃, 400 MHz): δ 11.35 (br s, 1H), 7.82 (br d, J=10.8 Hz, 1H), 7.69 (d, J=5.9 Hz, 1H), 7.56 (dd, J=9.0, 2.7 Hz, 1H), 7.44 (dd, J=9.3, 4.4 Hz, 1H), 7.16-7.27 (m, 1H), 4.01 (s, 3H), 2.65 (s, 3H).

### Step B: Methyl 2-((2-(1-((tert-butoxycarbonyl)(2-(6-methoxy-3-nitropyridin-2-yl)ethyl)-amino)ethyl)-4-fluorophenyl)amino)-5-fluoro-4-(trifluoromethyl)benzoate 117b

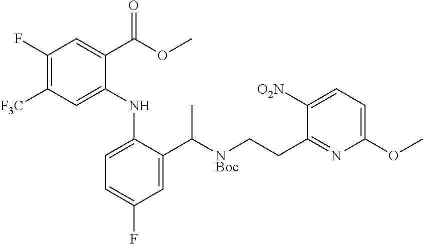 [SMILES: COC(=O)c1cc(F)c(C(F)(F)F)cc1Nc1ccc(F)cc1C(C)N(CCc1nc(OC)ccc1[N+](=O)[O-])C(=O)OC(C)(C)C]

Following the preparation outlined in Example 44, Step B, substituting methyl 5-fluoro-2-((4-fluoro-2-formylphenyl)amino)-4-(trifluoromethyl)benzoate with methyl 2-((2-acetyl-4-fluorophenyl)amino)-5-fluoro-4-(trifluoromethyl)benzoate 117a, methyl 2-((2-(1-((tert-butoxycarbonyl)(2-(6-methoxy-3-nitropyridin-2-yl)ethyl)amino)ethyl)-4-fluorophenyl)amino)-5-fluoro-4-(trifluoromethyl)benzoate 117b (2.80 g, 82% yield) was prepared as a yellow solid. HPLC/MS 1.71 min (B), [M+H]⁺ 655.3. ¹H NMR (CDCl₃, 400 MHz): δ 9.04 (s, 1H), 8.11 (d, J=8.8 Hz, 1H), 7.72 (d, J=10.8 Hz, 1H), 7.19-7.30 (m, 2H), 7.00 (td, J=8.2, 2.7 Hz, 1H), 6.90 (br d, J=4.4 Hz, 1H), 6.59 (d, J=9.3 Hz, 1H), 5.39-5.65 (m, 1H), 3.92 (s, 6H), 3.29-3.48 (m, 3H), 2.92-3.00 (m, 1H), 1.53 (d, J=6.8 Hz, 3H), 1.38 (s, 9H).

### Step C: 2-((2-(1-((tert-Butoxycarbonyl)(2-(6-methoxy-3-nitropyridin-2-yl)ethyl)amino)-ethyl)-4-fluorophenyl)amino)-5-fluoro-4-(trifluoromethyl)benzoic acid 117c

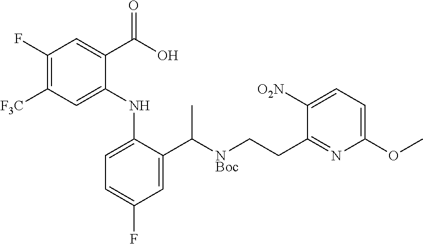 [SMILES: COc1ccc([N+](=O)[O-])c(CCN(C(=O)OC(C)(C)C)C(C)c2cc(F)ccc2Nc2cc(C(F)(F)F)c(F)cc2C(=O)O)n1]

Following the procedure outlined in Example 41, Step C, stirring the reaction mixture at ambient temperature for 2 h, 2-((2-(1-((tert-butoxycarbonyl)(2-(6-methoxy-3-nitropyridin-2-yl)ethyl)amino)ethyl)-4-fluorophenyl)amino)-5-fluoro-4-(trifluoromethyl)benzoic acid 117c (2.68 g, 95% yield) was prepared as a yellow powder. HPLC/MS 1.15 min (B), [M+H]⁺ 641.3. ¹H NMR (CDCl₃, 400 MHz): δ 9.01 (br s, 1H), 8.17 (br d, J=8.8 Hz, 1H), 7.81 (d, J=10.8 Hz, 1H), 7.23-7.31 (m, 2H), 7.07 (td, J=8.3, 2.9 Hz, 1H), 6.66 (br d, J=8.8 Hz, 1H), 6.57-6.93 (m, 1H), 5.57-5.90 (m, 1H), 3.91-4.03 (m, 3H), 3.27-3.53 (m, 3H), 2.94-3.10 (m, 1H), 1.58 (d, J=7.3 Hz, 3H), 1.31-1.42 (m, 9H).

### Step D: 2-((2-(1-((2-(3-Amino-6-methoxypyridin-2-yl)ethyl)(tert-butoxycarbonyl)-amino)ethyl)-4-fluorophenyl)amino)-5-fluoro-4-(trifluoromethyl)benzoic acid 117d

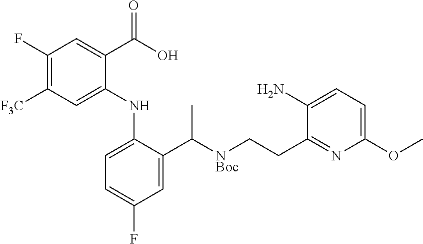 [SMILES: COc1ccc(N)c(CCN(C(=O)OC(C)(C)C)C(C)c2cc(F)ccc2Nc2cc(C(F)(F)F)c(F)cc2C(=O)O)n1]

Following the procedure outlined in Example 41, Step D, 2-((2-(1-((2-(3-amino-6-methoxypyridin-2-yl)ethyl)(tert-butoxycarbonyl)amino)ethyl)-4-fluorophenyl)amino)-5-fluoro-4-(trifluoromethyl)benzoic acid 117d (2.49 g, 98% yield) was prepared as a brown solid. HPLC/MS 1.07 min (A), [M+H]⁺ 611.3. ¹H NMR (CDCl₃, 400 MHz): δ 8.65-8.92 (m, 1H), 7.80 (d, J=11.2 Hz, 1H), 7.16 (br d, J=9.3 Hz, 2H), 7.00 (br d, J=5.4 Hz, 2H), 6.73 (br s, 1H), 6.36 (d, J=8.8 Hz, 1H), 5.10-5.31 (m, 1H), 3.66 (br s, 3H), 3.38-3.49 (m, 1H), 3.04-3.29 (m, 2H), 2.70 (dt, J=6.6, 3.1 Hz, 1H), 1.41-1.46 (m, 3H), 1.39 (s, 9H).

### Step E: tert-Butyl 2,8-difluoro-15-methoxy-10-methyl-19-oxo-3-(trifluoromethyl)-5,10,12,13,18,19-hexahydro-11H-dibenzo[c,f]pyrido[3,2-j][1,5,9]triazacyclotridecine-11-carboxylate 117e

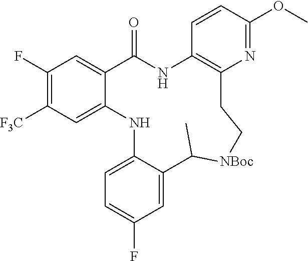 [SMILES: COc1ccc2c(n1)CCN(C(=O)OC(C)(C)C)C(C)c1cc(F)ccc1Nc1cc(C(F)(F)F)c(F)cc1C(=O)N2]

Following the procedure outlined in Example 41, Step E, stirring the reaction mixture at RT for 2 h, tert-butyl 2,8-difluoro-15-methoxy-10-methyl-19-oxo-3-(trifluoromethyl)-5,10,12,13,18,19-hexahydro-11H-dibenzo[c,f]pyrido[3,2-j][1,5,9]triazacyclo-tridecine-11-carboxylate 117e (1.79 g, 73% yield) was prepared as a white solid. HPLC/MS 1.51 min (B), [M+H]⁺ 593.3. ¹H NMR (CDCl₃, 400 MHz): δ 11.35 (br s, 1H), 8.34 (d, J=11.2 Hz, 1H), 8.23 (d, J=8.8 Hz, 1H), 7.58 (d, J=6.4 Hz, 1H), 7.12-7.22 (m, 1H), 6.92 (td, J=8.3, 2.9 Hz, 1H), 6.65 (d, J=8.8 Hz, 1H), 6.24 (br dd, J=8.8, 4.4 Hz, 1H), 6.09 (s, 1H), 5.80 (br d, J=6.4 Hz, 1H), 3.95 (dd, J=14.9, 11.5 Hz, 1H), 3.87 (s, 3H), 3.23 (br dd, J=15.2, 4.9 Hz, 1H), 2.30 (br dd, J=14.2, 5.4 Hz, 1H), 1.81 (br t, J=12.2 Hz, 1H), 1.60 (d, J=6.4 Hz, 3H), 0.94-1.19 (m, 9H).

### Step F: tert-Butyl 2,8-difluoro-15-methoxy-10-methyl-19-oxo-3-(trifluoromethyl)-12,13-dihydro-19H-5,18-methanodibenzo[c,f]pyrido[3,2-j][1,5,9]triazacyclotridecine-11(10H)-carboxylate 117f

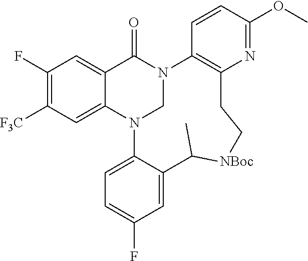 [SMILES: COc1ccc2c(n1)CCN(C(=O)OC(C)(C)C)C(C)c1cc(F)ccc1N1CN2C(=O)c2cc(F)c(C(F)(F)F)cc21]

Following the procedure outlined in Example 41, Step F, stirring the reaction at 90° C. for 23 h, tert-butyl 2,8-difluoro-15-methoxy-10-methyl-19-oxo-3-(trifluoromethyl)-12,13-dihydro-19H-5,18-methanodibenzo[c,f]pyrido[3,2-j][1,5,9]triazacyclotridecine-11(10H)-carboxylate 117f (1.16 g, 69% yield) was prepared as a light tan solid. HPLC/MS 1.50 min (A), [M+H]⁺ 605.3. ¹H NMR (CDCl₃, 400 MHz): δ 7.92 (d, J=9.8 Hz, 1H), 7.61 (dd, J=10.3, 2.9 Hz, 1H), 7.47 (d, J=8.8 Hz, 1H), 7.16-7.27 (m, 1H), 7.09-7.16 (m, 1H), 6.68 (d, J=8.3 Hz, 1H), 6.61 (d, J=5.4 Hz, 1H), 5.28 (d, J=10.8 Hz, 1H), 4.53 (q, J=6.8 Hz, 1H), 4.36 (d, J=10.8 Hz, 1H), 4.09-4.22 (m, 1H), 3.96 (s, 3H), 3.31-3.47 (m, 2H), 2.60-2.71 (m, 1H), 1.65 (d, J=6.8 Hz, 3H), 1.07-1.15 (m, 9H).

### Step G: 2,8-Difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione hydrochloride

### Example 117

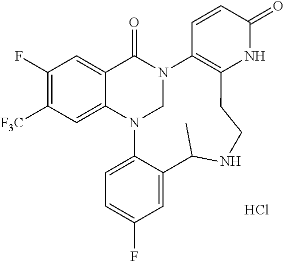 [SMILES: CC1NCCc2[nH]c(=O)ccc2N2CN(c3cc(C(F)(F)F)c(F)cc3C2=O)c2ccc(F)cc21 ; Cl]

Following the procedure outlined in Example 41, Step G, stirring the reaction mixture at 90° C. overnight, 2,8-difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione hydrochloride (1.01 g, 99% yield) was prepared as a white solid. HPLC/MS 1.01 min (B), [M+H]⁺ 491.2. ¹H NMR (DMSO-d₆, 400 MHz): δ 10.29-10.46 (m, 1H), 9.66-9.98 (m, 1H), 7.92 (d, J=10.3 Hz, 1H), 7.85 (dd, J=9.8, 2.9 Hz, 1H), 7.61 (dd, J=8.8, 5.4 Hz, 1H), 7.44-7.54 (m, 2H), 6.48 (d, J=5.4 Hz, 1H), 6.38 (d, J=9.8 Hz, 1H), 5.48 (d, J=11.7 Hz, 1H), 4.93 (d, J=11.7 Hz, 1H), 4.70 (br d, J=5.9 Hz, 1H), 3.28 (br d, J=7.8 Hz, 1H), 3.00 (br t, J=8.3 Hz, 1H), 2.69-2.90 (m, 2H), 1.53 (d, J=6.8 Hz, 3H).

### Example 118 and Example 119

### Step A: (10R)-2,8-Difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione (10S)-2,8-Difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione

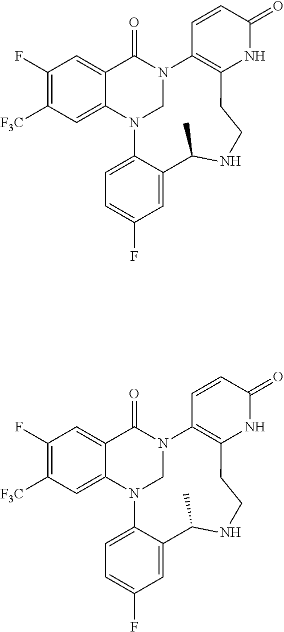 [SMILES: C[C@H]1NCCc2[nH]c(=O)ccc2N2CN(c3cc(C(F)(F)F)c(F)cc3C2=O)c2ccc(F)cc21 ; C[C@@H]1NCCc2[nH]c(=O)ccc2N2CN(c3cc(C(F)(F)F)c(F)cc3C2=O)c2ccc(F)cc21]

Racemic 2,8-difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione hydrochloride Example 117 (1.01 g, 1.92 mmol) was resolved using chiral chromatography (CC4 column, 150×4.6 mm, 5 μm), eluting with an isocratic gradient of methanol (0.1% isopropylamine) to afford:

First-eluting stereoisomer (10R)-2,8-difluoro-10-methyl-3-(trifluoromethyl)-10,11,12,13-tetrahydro-19H-5,18-methanodibenzo[c,f]pyrido[3,2-j][1,5,9]triazacyclotridecine-11(10H)-dione Example 118 (425 mg, 45% yield). HPLC/MS 1.01 min (B), [M+H]⁺ 491.1. ¹H NMR (DMSO-d₆, 400 MHz): δ 11.67-12.03 (m, 1H), 7.86 (d, J=10.3 Hz, 1H), 7.49 (dd, J=8.8, 5.4 Hz, 1H), 7.36-7.44 (m, 2H), 7.26 (td, J=8.4, 3.2 Hz, 1H), 6.56 (d, J=5.4 Hz, 1H), 6.22 (d, J=9.3 Hz, 1H), 5.42 (d, J=10.3 Hz, 1H), 4.89 (d, J=10.8 Hz, 1H), 4.00-4.17 (m, 1H), 2.93-3.04 (m, 1H), 2.66-2.79 (m, 1H), 2.51-2.59 (m, 1H), 2.22-2.30 (m, 1H), 1.23 (d, J=6.4 Hz, 3H).

Second-eluting stereoisomer (10S)-2,8-difluoro-10-methyl-3-(trifluoromethyl)-10,11,12,13-tetrahydro-19H-5,18-methanodibenzo[cl]pyrido[3,2-j][1,5,9]triazacyclotridecine-11(10H)-dione Example 119 (390 mg, 41% yield). HPLC/MS 1.02 min (B), [M+H]⁺ 491.2. ¹H NMR (DMSO-d₆, 400 MHz): δ 11.62-12.04 (m, 1H), 7.86 (d, J=10.3 Hz, 1H), 7.50 (dd, J=8.8, 5.4 Hz, 1H), 7.36-7.44 (m, 2H), 7.26 (td, J=8.4, 3.2 Hz, 1H), 6.56 (d, J=5.4 Hz, 1H), 6.22 (d, J=9.8 Hz, 1H), 5.42 (d, J=10.8 Hz, 1H), 4.89 (d, J=10.3 Hz, 1H), 4.02-4.16 (m, 1H), 2.93-3.05 (m, 1H), 2.67-2.79 (m, 1H), 2.51-2.59 (m, 1H), 2.21-2.31 (m, 1H), 1.23 (d, J=6.4 Hz, 3H).

### Example 120

### 3-Chloro-2,8-difluoro-10-methyl-10,11,12,13-tetrahydro-19H-5,18-methanodibenzo[c,f]pyrido-[3,2-j][1,5,9]triazacyclotridecine-15,19(14H)-dione hydrochloride

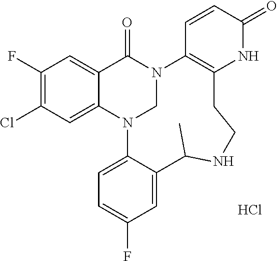 [SMILES: CC1NCCc2[nH]c(=O)ccc2N2CN(c3cc(Cl)c(F)cc3C2=O)c2ccc(F)cc21 ; Cl]

### Step A: Methyl 2-((2-acetyl-4-fluorophenyl)amino)-4-chloro-5-fluorobenzoate 120a

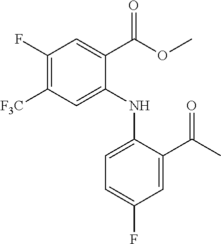 [SMILES: COC(=O)c1cc(F)c(C(F)(F)F)cc1Nc1ccc(F)cc1C(C)=O]

Following the preparation outlined in Example 7, Step A, substituting ethyl 2-bromo-5-(trifluoromethyl)benzoate with methyl 2-bromo-4-chloro-5-fluorobenzoate, Int-3a with 1-(2-amino-5-fluorophenyl)ethan-1-one, BINAP with Xantphos, toluene with 1,4-dioxane, and stirring the reaction at 80° C. for 23 h, methyl 2-((2-acetyl-4-fluorophenyl)amino)-4-chloro-5-fluorobenzoate 120a (39 mg, 35% yield) was prepared as a yellow solid. HPLC/MS 1.32 min (B), [M+H]⁺ 340.1. ¹H NMR (DMSO-d₆, 400 MHz): δ 10.87 (s, 1H), 7.80-7.88 (m, 2H), 7.49-7.56 (m, 2H), 7.38-7.45 (m, 1H), 3.88 (s, 3H), 2.62 (s, 3H).

### Step B: Methyl 4-chloro-5-fluoro-2-((4-fluoro-2-(1-((2-(6-methoxy-3-nitropyridin-2-yl)-ethyl)amino)ethyl)phenyl)amino)benzoate 120b

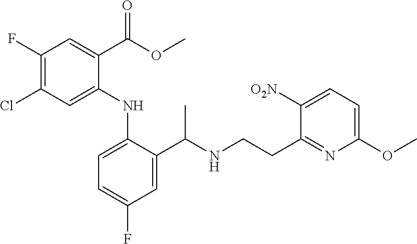 [SMILES: COC(=O)c1cc(F)c(Cl)cc1Nc1ccc(F)cc1C(C)NCCc1nc(OC)ccc1[N+](=O)[O-]]

To a solution of 2-(6-methoxy-3-nitropyridin-2-yl)ethan-1-amine bis-trifluoroacetic acid salt Int-1e (2.38 g, 5.60 mmol) in DCM (5 mL) was added TEA (4.26 mL, 30.6 mmol), the reaction mixture stirred for 5 min and concentrated under reduced pressure. THF (18 mL) was added to the residue followed by methyl 2-((2-acetyl-4-fluorophenyl)amino)-4-chloro-5-fluorobenzoate 120b (1.73 g, 5.09 mmol), and titanium (IV) ethoxide (2.44 g, 10.7 mmol) and the reaction mixture was stirred at 70° C. for 18 h, then at 75° C. for 4 h. The reaction mixture was cooled to ambient temperature, MeOH (18 mL) was added followed by the portion-wise addition of sodium cyanoborohydride (672 mg, 10.7 mmol) and the reaction mixture was stirred at ambient temperature for 18 h, concentrated under reduced pressure, suspended in DCM and H₂O, and stirred for 30 min. The mixture was filtered through celite, and the solid was rinsed with DCM. The biphasic filtrate was separated, the organic layer washed with brine, dried over Na₂SO₄, filtered, concentrated under reduced pressure. The crude residue was purified by silica gel flash column chromatography (120 g) eluting with a 100% heptanes to 40% EtOAc-heptanes gradient. Product fractions were combined and evaporated under reduced pressure to afford methyl 4-chloro-5-fluoro-2-((4-fluoro-2-(1-((2-(6-methoxy-3-nitropyridin-2-yl)ethyl)amino)ethyl)phenyl)-amino)benzoate 120b (1.47 g, 55% yield). HPLC/MS 0.96 min (A), [M+H]⁺ 521.2. ¹H NMR (DMSO-d₆, 400 MHz): δ 9.42 (s, 1H), 8.29 (d, J=8.8 Hz, 1H), 7.75 (d, J=9.8 Hz, 1H), 7.22-7.33 (m, 2H), 7.12 (td, J=8.4, 3.2 Hz, 1H), 6.83 (d, J=8.8 Hz, 1H), 6.72 (d, J=6.4 Hz, 1H), 3.82-3.92 (m, 7H), 3.06-3.24 (m, 2H), 2.72-2.82 (m, 2H), 1.20 (d, J=6.4 Hz, 3H).

### Step C: Methyl 2-((2-(1-((tert-butoxycarbonyl)(2-(6-methoxy-3-nitropyridin-2-yl)ethyl)-amino)ethyl)-4-fluorophenyl)amino)-4-chloro-5-fluorobenzoate 120c

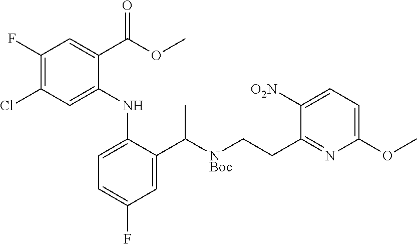 [SMILES: COC(=O)c1cc(F)c(Cl)cc1Nc1ccc(F)cc1C(C)N(CCc1nc(OC)ccc1[N+](=O)[O-])C(=O)OC(C)(C)C]

To methyl 4-chloro-5-fluoro-2-((4-fluoro-2-(1-((2-(6-methoxy-3-nitropyridin-2-yl)ethyl)-amino)ethyl)phenyl)amino)benzoate 120b (1.4 g, 2.71 mmol) in DCM (15 mL) was added BOC-anhydride (5.91 g, 27.1 mmol) in DCM (10 mL). The reaction mixture was stirred at ambient temperature for 66 h, concentrated under reduced pressure and purified by silica gel flash column chromatography (120 g) eluting with a 100% heptanes to 40% EtOAc-heptanes gradient. Product fractions were combined and evaporated under reduced pressure to afford methyl 2-((2-(1-((tert-butoxycarbonyl)(2-(6-methoxy-3-nitropyridin-2-yl)ethyl)amino)ethyl)-4-fluorophenyl)amino)-4-chloro-5-fluorobenzoate 120c (1.61 g, 96% yield). HPLC/MS 1.68 min (A), [M+H]⁺ 621.2. ¹H NMR (DMSO-d₆, 400 MHz): δ 8.87 (s, 1H), 8.27 (d, J=9.3 Hz, 1H), 7.77 (d, J=10.3 Hz, 1H), 7.40 (br d, J=7.8 Hz, 1H), 7.35 (dd, J=8.6, 5.6 Hz, 1H), 7.23 (td, J=8.3, 2.9 Hz, 1H), 6.85 (d, J=8.8 Hz, 1H), 6.60 (d, J=6.4 Hz, 1H), 5.27-5.48 (m, 1H), 3.91 (s, 3H), 3.86 (s, 3H), 3.35-3.47 (m, 2H), 3.10-3.23 (m, 1H), 2.72-2.85 (m, 1H), 1.44 (d, J=7.3 Hz, 3H), 1.26 (s, 9H).

### Step D: 2-((2-(1-((tert-Butoxycarbonyl)(2-(6-methoxy-3-nitropyridin-2-yl)ethyl-amino)ethyl)-4-fluorophenyl)amino)-4-chloro-5-fluorobenzoic acid hydrochloride 120d

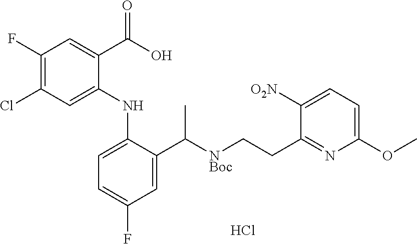 [SMILES: COc1ccc([N+](=O)[O-])c(CCN(C(=O)OC(C)(C)C)C(C)c2cc(F)ccc2Nc2cc(Cl)c(F)cc2C(=O)O)n1 ; Cl]

Following the procedure outlined in Example 41, Step C, stirring the reaction mixture at ambient temperature for 2 h, 2-((2-(1-((tert-butoxycarbonyl)(2-(6-methoxy-3-nitropyridin-2-yl)-ethyl)amino)ethyl)-4-fluorophenyl)amino)-4-chloro-5-fluorobenzoic acid hydrochloride 120d (21.6 g, 97% yield) was prepared as a foamy solid. HPLC/MS 1.58 min (A), [M+H]⁺ 607.2. ¹H NMR (DMSO-d₆, 400 MHz): δ 13.24-13.59 (m, 1H), 9.16 (br d, J=2.9 Hz, 1H), 8.23-8.32 (m, 1H), 7.74 (d, J=10.3 Hz, 1H), 7.38 (s, 1H), 7.32-7.37 (m, 1H), 7.22 (td, J=8.6, 2.9 Hz, 1H), 6.85 (d, J=8.8 Hz, 1H), 6.58 (d, J=6.4 Hz, 1H), 5.19-5.40 (m, 1H), 3.91 (s, 3H), 3.36-3.44 (m, 2H), 3.21 (br dd, J=13.9, 7.6 Hz, 1H), 2.79 (br dd, J=13.9, 7.1 Hz, 1H), 1.44 (d, J=7.3 Hz, 3H), 1.25 (s, 9H).

### Step E: 2-((2-(1-((2-(3-Amino-6-methoxypyridin-2-yl)ethyl)(tert-butoxycarbonyl)-amino)ethyl)-4-fluorophenyl)amino)-4-chloro-5-fluorobenzoic acid 120e

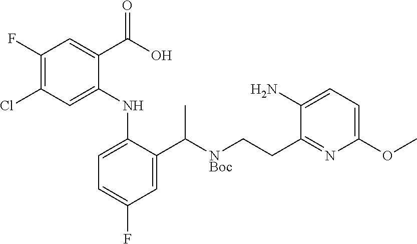 [SMILES: COc1ccc(N)c(CCN(C(=O)OC(C)(C)C)C(C)c2cc(F)ccc2Nc2cc(Cl)c(F)cc2C(=O)O)n1]

Following the procedure outlined in Example 41, Step D, 2-((2-(1-((2-(3-amino-6-methoxypyridin-2-yl)ethyl)(tert-butoxycarbonyl)amino)ethyl)-4-fluorophenyl)amino)-4-chloro-5-fluorobenzoic acid 120e (1.50 g, 100% yield) was prepared. HPLC/MS 1.16 min (A), [M+H]⁺ 577.2. ¹H NMR (DMSO-d₆, 400 MHz): δ 7.75 (d, J=10.3 Hz, 1H), 7.24 (br d, J=5.4 Hz, 1H), 7.22 (br d, J=5.4 Hz, 1H), 7.12 (td, J=8.3, 2.9 Hz, 1H), 6.99 (d, J=8.8 Hz, 1H), 6.45 (d, J=5.9 Hz, 1H), 6.39 (d, J=8.3 Hz, 1H), 5.27 (br d, J=2.9 Hz, 1H), 4.47 (br s, 2H), 3.66 (s, 3H), 3.38 (br s, 2H), 2.64-2.75 (m, 1H), 2.37-2.49 (m, 1H), 1.38 (br d, J=7.3 Hz, 3H), 1.22 (br s, 9H).

### Step F: tert-Butyl 3-chloro-2,8-difluoro-15-methoxy-10-methyl-19-oxo-5,10,12,13,18,19-hexahydro-11H-dibenzo[c,f]pyrido[3,2-j][1,5,9]triazacyclotridecine-11-carboxylate 120f

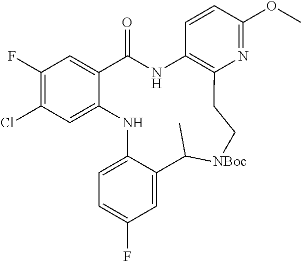 [SMILES: COc1ccc2c(n1)CCN(C(=O)OC(C)(C)C)C(C)c1cc(F)ccc1Nc1cc(Cl)c(F)cc1C(=O)N2]

Following the procedure outlined in Example 44, Step E, stirring the reaction mixture at RT for 4 h, tert-butyl 3-chloro-2,8-difluoro-15-methoxy-10-methyl-19-oxo-5,10,12,13,18,19-hexahydro-11H-dibenzo[c,f]pyrido[3,2-j][1,5,9]triazacyclotridecine-11-carboxylate 120f (715 mg, 53% yield) was prepared. HPLC/MS 1.38 min (A), [M+H]⁺ 559.2. ¹H NMR (DMSO-d₆, 400 MHz): δ 10.87 (br s, 1H), 8.02 (d, J=10.3 Hz, 1H), 7.71 (br d, J=8.8 Hz, 1H), 7.64 (br s, 1H), 7.56 (d, J=6.8 Hz, 1H), 7.46 (dd, J=9.8, 2.9 Hz, 1H), 7.04 (td, J=8.6, 2.9 Hz, 1H), 6.68 (d, J=8.8 Hz, 1H), 6.26-6.38 (m, 1H), 5.78-5.94 (m, 1H), 3.79 (s, 3H), 3.65-3.74 (m, 1H), 3.19-3.28 (m, 1H), 2.06 (br d, J=10.3 Hz, 1H), 1.54-1.67 (m, 1H), 1.46 (d, J=6.8 Hz, 3H), 0.99 (br s, 9H).

### Step G: tert-Butyl 3-chloro-2,8-difluoro-15-methoxy-10-methyl-19-oxo-12,13-dihydro-19H-5,18-methanodibenzo[c,f]pyrido[3,2-j][1,5,9]triazacyclotridecine-11(10H)-carboxylate 1202

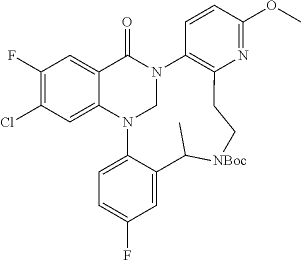 [SMILES: COc1ccc2c(n1)CCN(C(=O)OC(C)(C)C)C(C)c1cc(F)ccc1N1CN2C(=O)c2cc(F)c(Cl)cc21]

Following the procedure outlined in Example 41, Step F, stirring the reaction at 85° C. for 36 h, tert-butyl 3-chloro-2,8-difluoro-15-methoxy-10-methyl-19-oxo-12,13-dihydro-19H-5,18-methanodibenzo[c,f]pyrido[3,2-j][1,5,9]triazacyclotridecine-11(10H)-carboxylate 120g (575 mg, 79% yield) was prepared as a white solid. HPLC/MS 1.36 min (A), [M+H]⁺ 571.1. ¹H NMR (DMSO-d₆, 400 MHz): δ 7.78 (d, J=8.8 Hz, 1H), 7.63 (d, J=8.3 Hz, 1H), 7.51 (dd, J=11.2, 2.9 Hz, 1H), 7.44 (dd, J=8.8, 5.9 Hz, 1H), 7.29 (td, J=8.3, 2.9 Hz, 1H), 6.75 (d, J=8.3 Hz, 1H), 6.39 (d, J=5.9 Hz, 1H), 5.42 (d, J=10.8 Hz, 1H), 4.54 (q, J=6.7 Hz, 1H), 4.11 (d, J=10.3 Hz, 1H), 3.94-4.05 (m, 1H), 3.87 (s, 3H), 3.36-3.43 (m, 1H), 3.15-3.26 (m, 1H), 2.39-2.48 (m, 1H), 1.53 (d, J=6.4 Hz, 3H), 0.97 (s, 9H).

### Step H: 3-Chloro-2,8-difluoro-10-methyl-10,11,12,13-tetrahydro-19H-5,18-methano-dibenzo[c,f]pyrido[3,2-j][1,5,9]triazacyclotridecine-15,19(14H)-dione hydrochloride

### Example 120

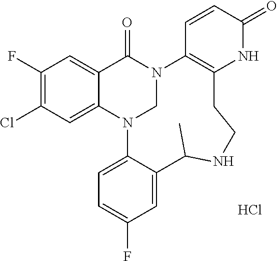 [SMILES: CC1NCCc2[nH]c(=O)ccc2N2CN(c3cc(Cl)c(F)cc3C2=O)c2ccc(F)cc21 ; Cl]

Following the procedure outlined in Example 41, Step G, stirring the reaction mixture at 85° C. for 18 h, 3-chloro-2,8-difluoro-10-methyl-10,11,12,13-tetrahydro-19H-5,18-methano-dibenzo[c,f]pyrido[3,2-j][1,5,9]triazacyclotridecine-15,19(14H)-dione hydrochloride (490 mg, 100% yield) was prepared. HPLC/MS 0.65 min (A), [M+H]⁺ 457.2. ¹H NMR (DMSO-d₆, 400 MHz): δ 9.96-10.16 (m, 1H) 9.45-9.66 (m, 1H) 7.82 (d, J=8.80 Hz, 1H) 7.75 (dd, J=9.78, 2.93 Hz, 1H) 7.57 (dd, J=8.80, 5.87 Hz, 1H) 7.49 (dd, J=7.83, 2.93 Hz, 1H) 7.45 (d, J=9.78 Hz, 1H) 6.46 (d, J=5.87 Hz, 1H) 6.32 (d, J=9.29 Hz, 1H) 5.44 (d, J=11.74 Hz, 1H) 4.84 (d, J=11.74 Hz, 1H) 4.67 (br s, 1H) 3.19-3.29 (m, 1H) 2.94-3.04 (m, 1H) 2.77-2.86 (m, 1H) 2.67-2.75 (m, 1H) 1.54 (d, J=6.85 Hz, 3H).

### Example 121 and Example 122

### Step A: (10R)-3-Chloro-2,8-difluoro-10-methyl-10,11,12,13-tetrahydro-19H-5,18-methanodibenzo[c,f]pyrido[3,2-j][1,5,9]triazacyclotridecine-15,19(14H)-dione (10S)-3-Chloro-2,8-difluoro-10-methyl-10,11,12,13-tetrahydro-19H-5,18-methano-dibenzo[c,f]pyrido[3,2-j][1,5,9]triazacyclotridecine-15,19(14H)-dione

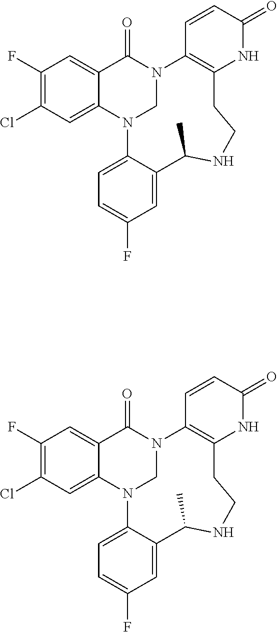 [SMILES: C[C@H]1NCCc2[nH]c(=O)ccc2N2CN(c3cc(Cl)c(F)cc3C2=O)c2ccc(F)cc21 ; C[C@@H]1NCCc2[nH]c(=O)ccc2N2CN(c3cc(Cl)c(F)cc3C2=O)c2ccc(F)cc21]

Racemic 3-chloro-2,8-difluoro-10-methyl-10,11,12,13-tetrahydro-19H-5,18-methano-dibenzo[c,f]pyrido[3,2-j][1,5,9]triazacyclotridecine-15,19(14H)-dione hydrochloride Example 120 was resolved using chiral chromatography (CC4 column, 150×4.6 mm, 5 μm), eluting with an isocratic gradient of methanol (0.1% isopropylamine) to afford:

First-eluting stereoisomer (10R)-3-chloro-2,8-difluoro-10-methyl-10,11,12,13-tetrahydro-19H-5,18-methanodibenzo[c,f]pyrido[3,2-j][1,5,9]triazacyclotridecine-15,19(14H)-dione Example 121. HPLC/MS 0.65 min (A), [M+H]⁺ 457.1. ¹H NMR (400 MHz, DMSO-d₆): δ 11.78 (br s, 1H), 7.77 (d, J=9.29 Hz, 1H), 7.45 (dd, J=8.56, 5.62 Hz, 1H), 7.35-7.41 (m, 2H), 7.25 (td, J=8.44, 3.18 Hz, 1H), 6.45 (d, J=5.87 Hz, 1H), 6.20 (d, J=9.78 Hz, 1H), 5.38 (d, J=10.76 Hz, 1H), 4.82 (d, J=10.76 Hz, 1H), 4.08 (br s, 1H), 2.91-3.04 (m, 1H), 2.65-2.77 (m, 1H), 2.12-2.31 (m, 2H), 1.24 (d, J=6.36 Hz, 3H).

Second-eluting stereoisomer (10S)-3-chloro-2,8-difluoro-10-methyl-10,11,12,13-tetrahydro-19H-5,18-methanodibenzo[c,f]pyrido[3,2-j][1,5,9]triazacyclotridecine-15,19(14H)-dione Example 122. HPLC/MS 0.66 min (A), [M+H]⁺ 457.1. ¹H NMR (400 MHz, DMSO-d₆): δ 11.79 (br s, 1H), 7.77 (d, J=8.80 Hz, 1H), 7.45 (dd, J=8.56, 5.62 Hz, 1H), 7.35-7.41 (m, 2H), 7.25 (td, J=8.56, 2.93 Hz, 1H), 6.45 (d, J=5.87 Hz, 1H), 6.20 (d, J=9.29 Hz, 1H), 5.38 (d, J=10.76 Hz, 1H), 4.82 (d, J=10.76 Hz, 1H), 4.05-4.14 (m, 1H), 2.93-3.04 (m, 1H), 2.66-2.77 (m, 1H), 2.15-2.30 (m, 2H), 1.24 (d, J=6.36 Hz, 3H).

### Example 123

### 2,8-Difluoro-12-methyl-3-(trifluoromethyl)-10,11,12,13-tetrahydro-19H-5,18-methanodibenzo-[c,f]pyrido[3,2-j][1,5,9]triazacyclotridecine-15,19(14H)-dione hydrochloride

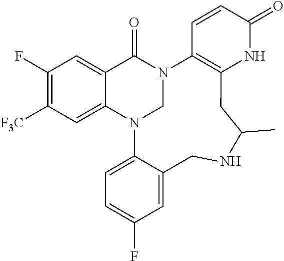 [SMILES: CC1Cc2[nH]c(=O)ccc2N2CN(c3ccc(F)cc3CN1)c1cc(C(F)(F)F)c(F)cc1C2=O]

### Step A: tert-Butyl (2-bromo-5-fluorobenzyl)(1-(6-methoxy-3-nitropyridin-2-yl)propan-2-yl)carbamate 123a

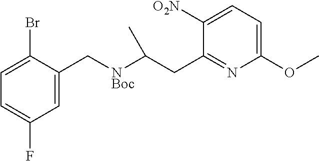 [SMILES: COc1ccc([N+](=O)[O-])c(CC(C)N(Cc2cc(F)ccc2Br)C(=O)OC(C)(C)C)n1]

Following the preparation outlined in Example 44, Step B, using (2-bromo-5-fluorophenyl)methanamine and 1-(6-methoxy-3-nitropyridin-2-yl)propan-2-one Int-1i as substrates, tert-butyl (2-bromo-5-fluorobenzyl)(1-(6-methoxy-3-nitropyridin-2-yl)propan-2-yl)carbamate 123a (6.78 g, 72% yield) was prepared as a light orange solid. HPLC/MS 1.52 min (B), [M+H]⁺ 498.1/500.1. ¹H NMR (DMSO-d₆, 400 MHz): δ 8.3-8.4 (m, 1H), 7.63 (dd, 1H, J=5.4, 8.8 Hz), 7.07 (dt, 1H, J=3.2, 8.4 Hz), 6.90 (br dd, 1H, J=8.6, 16.9 Hz), 6.80 (br s, 1H), 4.7-5.0 (m, 1H), 4.5-4.6 (m, 1H), 4.28 (br d, 1H, J=17.1 Hz), 4.0-4.1 (m, 3H), 3.3-3.4 (m, 1H), 3.0-3.2 (m, 1H), 1.2-1.3 (m, 3H), 1.0-1.2 (m, 9H) (rotamers).

### Step B: Methyl 2-((2-(((tert-butoxycarbonyl)(1-(6-methoxy-3-nitropyridin-2-yl)propan-2-yl)amino)methyl)-4-fluorophenyl)amino)-5-fluoro-4-(trifluoromethyl)benzoate 123b

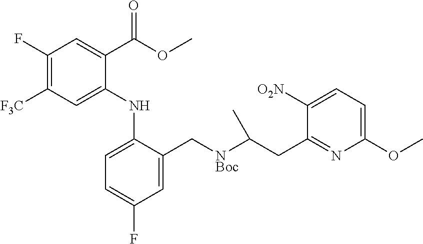 [SMILES: COC(=O)c1cc(F)c(C(F)(F)F)cc1Nc1ccc(F)cc1CN(C(=O)OC(C)(C)C)C(C)Cc1nc(OC)ccc1[N+](=O)[O-]]

Following the preparation outlined in Example 7, Step A, substituting ethyl 2-bromo-5-(trifluoromethyl)benzoate with tert-butyl (2-bromo-5-fluorobenzyl)(1-(6-methoxy-3-nitropyridin-2-yl)propan-2-yl)carbamate 123a, Int-3a with methyl 2-amino-5-fluoro-4-(trifluoromethyl)benzoate, BINAP with Xantphos, toluene with 1,4-dioxane, and stirring the reaction at 90° C. for 20 h, methyl 2-((2-(((tert-butoxycarbonyl)(1-(6-methoxy-3-nitropyridin-2-yl)propan-2-yl)amino)methyl)-4-fluorophenyl)amino)-5-fluoro-4-(trifluoromethyl)benzoate 123b (670 mg, 8% yield) was prepared as a light yellow solid. HPLC/MS 1.67 min (B), [M+H]⁺ 355.1. ¹H NMR (DMSO-d₆, 400 MHz): δ 8.96 (s, 1H), 8.31 (br s, 1H), 7.88 (br d, 1H, J=11.2 Hz), 7.33 (br s, 1H), 7.16 (dt, 1H, J=2.9, 8.3 Hz), 6.8-7.0 (m, 2H), 6.6-6.7 (m, 1H), 4.5-4.9 (m, 1H), 4.29 (br s, 2H), 3.94 (s, 3H), 3.69 (s, 3H), 3.2-3.3 (m, 1H), 2.91 (dd, 1H, J=10.8, 13.2 Hz), 0.9-1.3 (m, 12H).

### Step C: 2-((2-(((tert-Butoxycarbonyl)(1-(6-methoxy-3-nitropyridin-2-yl)propan-2-yl)-amino)methyl)-4-fluorophenyl)amino)-5-fluoro-4-(trifluoromethyl)benzoic acid 123c

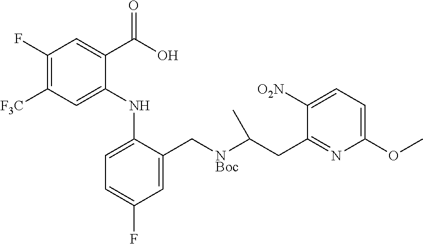 [SMILES: COc1ccc([N+](=O)[O-])c(CC(C)N(Cc2cc(F)ccc2Nc2cc(C(F)(F)F)c(F)cc2C(=O)O)C(=O)OC(C)(C)C)n1]

Following the procedure outlined in Example 41, Step C, stirring the reaction mixture at 50° C. for 20 h, 2-((2-(((tert-butoxycarbonyl)(1-(6-methoxy-3-nitropyridin-2-yl)propan-2-yl)amino)methyl)-4-fluorophenyl)amino)-5-fluoro-4-(trifluoromethyl)benzoic acid 123c (5.71 g, 100% yield) was prepared as a light brown solid. HPLC/MS 1.13 min (B), [M+H]⁺ 641.1. ¹H NMR (DMSO-d₆, 400 MHz): δ 9.26 (br s, 1H), 8.33 (br s, 1H), 7.87 (br d, 1H, J=10.8 Hz), 7.36 (br s, 1H), 7.16 (dt, 1H, J=2.9, 8.3 Hz), 6.87 (br s, 2H), 6.6-6.8 (m, 1H), 4.5-4.9 (m, 1H), 4.31 (br s, 2H), 3.71 (s, 3H), 3.3-3.4 (m, 2H), 2.90 (dd, 1H, J=10.8, 13.2 Hz), 0.9-1.2 (m, 12H).

### Step D: 2-((2-(((1-(3-Amino-6-methoxypyridin-2-yl)propan-2-yl)(tert-butoxycarbonyl)-amino)methyl)-4-fluorophenyl)amino)-5-fluoro-4-(trifluoromethyl)benzoic acid 123d

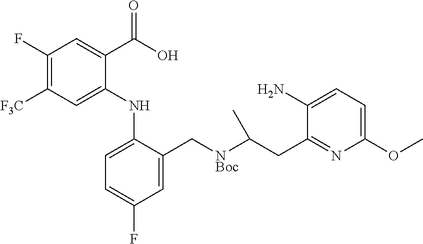 [SMILES: COc1ccc(N)c(CC(C)N(Cc2cc(F)ccc2Nc2cc(C(F)(F)F)c(F)cc2C(=O)O)C(=O)OC(C)(C)C)n1]

Following the procedure outlined in Example 44, Step D, 2-((2-(((1-(3-amino-6-methoxypyridin-2-yl)propan-2-yl)(tert-butoxycarbonyl)amino)methyl)-4-fluorophenyl)amino)-5-fluoro-4-(trifluoromethyl)benzoic acid 123d (5.28 g, 92% yield) was prepared as a brown solid. HPLC/MS 1.03 min (B), [M+H]⁺ 611.2. ¹H NMR (DMSO-d₆, 400 MHz): δ 9.29 (br s, 1H), 7.86 (d, 1H, J=11.2 Hz), 7.3-7.4 (m, 1H), 7.1-7.2 (m, 1H), 6.93 (br d, 2H, J=8.8 Hz), 6.6-6.7 (m, 1H), 6.34 (d, 1H, J=8.8 Hz), 4.57 (br d, 1H, J=8.8 Hz), 4.32 (br s, 1H), 4.0-4.3 (m, 2H), 3.47 (s, 3H), 3.2-3.4 (m, 1H), 2.7-2.7 (m, 1H), 1.0-1.3 (m, 12H) (two protons obscured).

### Step E: tert-Butyl 2,8-difluoro-15-methoxy-12-methyl-19-oxo-3-(trifluoromethyl)-5,10,12,13,18,19-hexahydro-11H-dibenzo[c,f]pyrido[3,2-j][1,5,9]triazacyclotridecine-11-carboxylate 123e

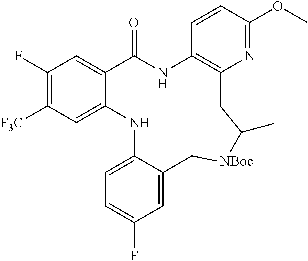 [SMILES: COc1ccc2c(n1)CC(C)N(C(=O)OC(C)(C)C)Cc1cc(F)ccc1Nc1cc(C(F)(F)F)c(F)cc1C(=O)N2]

Following the procedure outlined in Example 42, Step E, substituting HATU with HOAT/EDC and stirring at room temperature for 18 h, tert-butyl 2,8-difluoro-15-methoxy-12-methyl-19-oxo-3-(trifluoromethyl)-5,10,12,13,18,19-hexahydro-11H-dibenzo[c,f]pyrido[3,2-j][1,5,9]triazacyclotridecine-11-carboxylate 123e (714 mg, 16% yield) was prepared as a light yellow solid. HPLC/MS 1.51 min (B), [M+H]⁺ 593.2. ¹H NMR (DMSO-d₆, 400 MHz): δ 9.66 (s, 1H), 7.93 (d, 1H, J=8.8 Hz), 7.6-7.7 (m, 2H), 7.37 (br s, 1H), 6.7-6.9 (m, 2H), 6.67 (dd, 1H, J=4.9, 8.8 Hz), 6.55 (d, 1H, J=8.8 Hz), 4.3-4.5 (m, 3H), 3.81 (s, 3H), 2.9-3.0 (m, 1H), 2.72 (br s, 1H), 1.46 (s, 9H), 1.08 (br d, 3H, J=6.4 Hz).

### Step F: tert-Butyl 2,8-difluoro-15-methoxy-12-methyl-19-oxo-3-(trifluoromethyl)-12,13-dihydro-19H-5,18-methanodibenzo[c,f]pyrido[3,2-j][1,5,9]triazacyclotridecine-11(10H)-carboxylate 123f

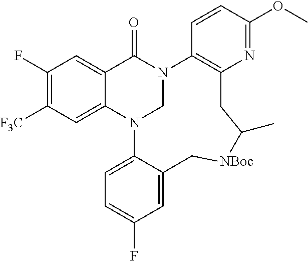 [SMILES: COc1ccc2c(n1)CC(C)N(C(=O)OC(C)(C)C)Cc1cc(F)ccc1N1CN2C(=O)c2cc(F)c(C(F)(F)F)cc21]

Following the procedure outlined in Example 41, Step F, stirring the reaction at 90° C. for 64 h, tert-butyl 2,8-difluoro-15-methoxy-12-methyl-19-oxo-3-(trifluoromethyl)-12,13-dihydro-19H-5,18-methanodibenzo[c,f]pyrido[3,2-j][1,5,9]triazacyclotridecine-11(10H)-carboxylate 123f (506 mg, 70% yield) was prepared as a yellow solid. HPLC/MS 1.45 min (A), [M+H]⁺ 605.2. ¹H NMR (DMSO-d₆, 400 MHz): δ 7.8-7.9 (m, 1H), 7.7-7.8 (m, 1H), 7.5-7.5 (m, 1H), 7.2-7.4 (m, 2H), 6.78 (d, 1H, J=8.3 Hz), 6.4-6.6 (m, 1H), 5.5-5.6 (m, 1H), 4.5-4.6 (m, 1H), 4.38 (br d, 1H, J=13.7 Hz), 4.0-4.0 (m, 1H), 3.8-3.9 (m, 3H), 3.69 (br d, 1H, J=14.7 Hz), 2.9-3.1 (m, 1H), 2.5-2.7 (m, 1H), 1.2-1.5 (m, 9H), 1.0-1.2 (m, 3H).

### Step G: 2,8-Difluoro-12-methyl-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

### Example 123

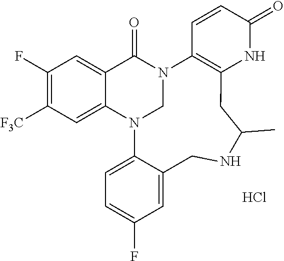 [SMILES: CC1Cc2[nH]c(=O)ccc2N2CN(c3ccc(F)cc3CN1)c1cc(C(F)(F)F)c(F)cc1C2=O ; Cl]

Following the procedure outlined in Example 41, Step G, stirring the reaction mixture at 90° C. for 7 h, 2,8-difluoro-12-methyl-3-(trifluoromethyl)-10,11,12,13-tetrahydro-19H-5,18-methano-dibenzo[c,f]pyrido[3,2-j][1,5,9]triazacyclotridecine-15,19(14H)-dione hydrochloride (310 mg, 70% yield) was prepared as a white solid. HPLC/MS 1.01 min (B), [M+H]⁺ 491.2. ¹H NMR (DMSO-d₆, 400 MHz): δ 11.91 (br s, 1H), 10.2-10.6 (m, 1H), 8.56 (br s, 1H), 7.92 (d, 1H, J=10.3 Hz), 7.79 (dd, 1H, J=2.9, 9.3 Hz), 7.70 (dd, 1H, J=5.4, 8.8 Hz), 7.4-7.6 (m, 2H), 6.44 (br d, 1H, J=5.4 Hz), 6.34 (br s, 1H), 5.45 (br d, 1H, J=11.2 Hz), 4.99 (d, 1H, J=11.7 Hz), 4.23 (br s, 1H), 4.03 (br d, 1H, J=7.3 Hz), 3.56 (br s, 1H), 3.17 (s, 1H), 2.4-2.5 (m, 1H), 1.35 (br d, 3H, J=6.4 Hz).

### Example 124 and Example 125

### Step A: 2,8-Difluoro-12-methyl-3-(trifluoromethyl)-10,11,12,13-tetrahydro-19H-5,18-methanodibenzo[c,f]pyrido[3,2-j][1,5,9]triazacyclotridecine-15,19(14H)-dione, Isomer 1

### 2,8-Difluoro-12-methyl-3-(trifluoromethyl)-10,11,12,13-tetrahydro-19H-5,18-methano-dibenzo[c,f]pyrido[3,2-j][1,5,9]triazacyclotridecine-15,19(14H)-dione, Isomer 2

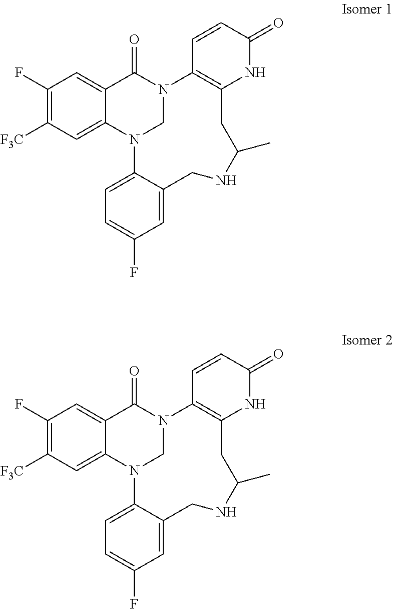 [SMILES: CC1Cc2[nH]c(=O)ccc2N2CN(c3ccc(F)cc3CN1)c1cc(C(F)(F)F)c(F)cc1C2=O ; CC1Cc2[nH]c(=O)ccc2N2CN(c3ccc(F)cc3CN1)c1cc(C(F)(F)F)c(F)cc1C2=O]

Racemic 2,8-difluoro-12-methyl-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 123 was resolved using chiral chromatography (SFC) to afford four peaks: molecules with different chirality, but each having two atropoisomers. Peaks (in elution order) 1 and 4 possess the same chirality as do peaks 2 and 3:

Fourth-eluting isomer: 2,8-difluoro-12-methyl-3-(trifluoromethyl)-10,11,12,13-tetrahydro-19H-5,18-methanodibenzo[c,f]pyrido[3,2-j][1,5,9]triazacyclotridecine-15,19(14H)-dione, Isomer 1 Example 124. HPLC/MS 1.01 min (B), [M+H]⁺ 491.1. ¹H NMR (DMSO-d₆, 400 MHz): δ 11.5-12.0 (m, 1H), 7.8-7.9 (m, 1H), 7.6-7.7 (m, 1H), 7.4-7.5 (m, 2H), 7.3-7.3 (m, 1H), 6.6-6.7 (m, 1H), 6.2-6.3 (m, 1H), 5.4-5.5 (m, 1H), 5.0-5.2 (m, 1H), 3.7-3.9 (m, 1H), 3.4-3.5 (m, 1H), 2.9-3.1 (m, 2H), 2.1-2.3 (m, 1H), 1.7-2.1 (m, 1H), 1.0-1.2 (m, 3H).

Third-eluting isomer: 2,8-difluoro-12-methyl-3-(trifluoromethyl)-10,11,12,13-tetrahydro-19H-5,18-methanodibenzo[c,f]pyrido[3,2-j][1,5,9]triazacyclotridecine-15,19(14H)-dione, Isomer 2 Example 125. HPLC/MS 1.01 min (B), [M+H]⁺ 491.1. ¹H NMR (DMSO-d₆, 400 MHz): δ 11.5-12.0 (m, 1H), 7.8-7.9 (m, 1H), 7.6-7.7 (m, 1H), 7.4-7.5 (m, 2H), 7.3-7.3 (m, 1H), 6.6-6.6 (m, 1H), 6.2-6.3 (m, 1H), 5.4-5.5 (m, 1H), 5.0-5.2 (m, 1H), 3.7-3.9 (m, 1H), 3.4-3.5 (m, 1H), 2.9-3.1 (m, 2H), 2.1-2.3 (m, 1H), 1.7-2.1 (m, 1H), 1.0-1.2 (m, 3H).

### Example 126

### 10-Ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride

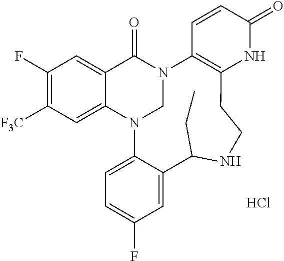 [SMILES: CCC1NCCc2[nH]c(=O)ccc2N2CN(c3cc(C(F)(F)F)c(F)cc3C2=O)c2ccc(F)cc21 ; Cl]

### Step A: Methyl 5-fluoro-2-((4-fluoro-2-propionylphenyl)amino)-4-(trifluoromethyl)-benzoate 126a

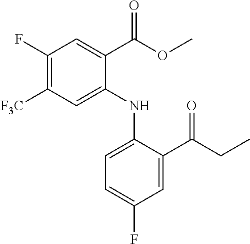 [SMILES: CCC(=O)c1cc(F)ccc1Nc1cc(C(F)(F)F)c(F)cc1C(=O)OC]

Following the preparation outlined in Example 7, Step A, substituting ethyl 2-bromo-5-(trifluoromethyl)benzoate with methyl 2-bromo-5-fluoro-4-(trifluoromethyl)benzoate, Int-3a with 1-(2-amino-5-fluorophenyl)propan-1-one, BINAP with Xantphos, toluene with 1,4-dioxane, and stirring the reaction at 90° C. for 18 h, methyl 5-fluoro-2-((4-fluoro-2-propionylphenyl)amino)-4-(trifluoromethyl)benzoate 126a (3.47 g, 65% yield) was prepared as a yellow solid. HPLC/MS 1.43 min (B), [M+H]⁺ 388.1. ¹H NMR (DMSO-d₆, 400 MHz): δ 10.90 (s, 1H), 7.90 (d, 1H, J=11.2 Hz), 7.86 (dd, 1H, J=2.9, 9.8 Hz), 7.61 (d, 1H, J=6.4 Hz), 7.55 (dd, 1H, J=4.9, 8.8 Hz), 7.41 (ddd, 1H, J=2.9, 7.9, 9.2 Hz), 3.92 (s, 3H), 3.08 (q, 2H, J=7.2 Hz), 1.0-1.1 (m, 3H).

### Step B: Methyl 5-fluoro-2-((4-fluoro-2-(1-((2-(6-methoxy-3-nitropyridin-2-yl)ethyl)-amino)propyl)phenyl)amino)-4-(trifluoromethyl)benzoate 126b

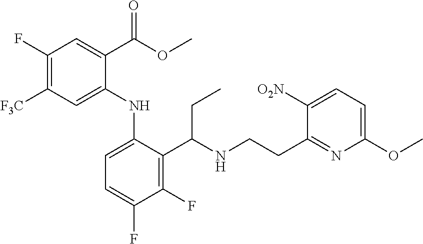 [SMILES: CCC(NCCc1nc(OC)ccc1[N+](=O)[O-])c1c(Nc2cc(C(F)(F)F)c(F)cc2C(=O)OC)ccc(F)c1F]

Following the preparation outlined in Example 120, Step B, using methyl 5-fluoro-2-((4-fluoro-2-propionylphenyl)amino)-4-(trifluoromethyl)benzoate 126a and 2-(6-methoxy-3-nitropyridin-2-yl)ethan-1-amine, TFA salt Int-1e as substrates, methyl 5-fluoro-2-((4-fluoro-2-(1-((2-(6-methoxy-3-nitropyridin-2-yl)ethyl)amino)propyl)phenyl)amino)-4-(trifluoromethyl)-benzoate 126b (2.23 g, 410% yield) was prepared as a yellow oil. HPLC/MS 1.63 min (B), [M+H]⁺ 569.1. ¹H NMR (DMSO-d₆, 400 MHz): δ 9.46 (s, 1H), 8.2-8.3 (m, 1H), 7.8-7.9 (m, 1H), 7.33 (dd, 1H, J=5.4, 8.8 Hz), 7.2-7.3 (m, 1H), 7.1-7.2 (m, 1H), 6.8-6.9 (m, 2H), 3.88 (s, 3H), 3.82 (s, 3H), 3.6-3.7 (m, 1H), 3.0-3.2 (m, 2H), 2.7-2.8 (m, 2H), 2.3-2.5 (m, 1H), 1.4-1.7 (m, 2H), 0.70 (t, 3H, J=7.3 Hz).

### Step C: Methyl 2-((2-(1-((tert-butoxycarbonyl)(2-(6-methoxy-3-nitropyridin-2-yl)ethyl)-amino)propyl)-4-fluorophenyl)amino)-5-fluoro-4-(trifluoromethyl)benzoate 126c

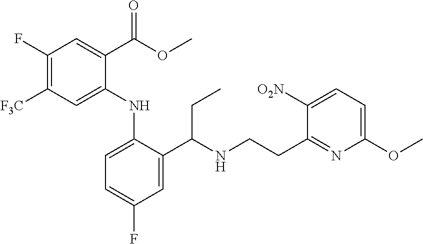 [SMILES: CCC(NCCc1nc(OC)ccc1[N+](=O)[O-])c1cc(F)ccc1Nc1cc(C(F)(F)F)c(F)cc1C(=O)OC]

Following the preparation outlined in Example 120, Step C, stirring at 70° C. for 18 h, methyl 2-((2-(1-((tert-butoxycarbonyl)(2-(6-methoxy-3-nitropyridin-2-yl)ethyl)amino)propyl)-4-fluorophenyl)amino)-5-fluoro-4-(trifluoromethyl)benzoate 126c (2.20 g, 96% yield) was prepared as a yellow solid. HPLC/MS 1.71 min (B), [M+H]⁺ 669.2. ¹H NMR (DMSO-d₆, 400 MHz): δ 8.90 (s, 1H), 8.27 (d, 1H, J=8.8 Hz), 7.83 (br d, 1H, J=10.3 Hz), 7.46 (dd, 1H, J=2.9, 9.8 Hz), 7.37 (dd, 1H, J=5.4, 8.3 Hz), 7.26 (dt, 1H, J=2.7, 8.4 Hz), 6.85 (d, 1H, J=8.8 Hz), 6.56 (d, 1H, J=5.9 Hz), 5.30 (brs, 1H), 3.92 (s, 3H), 3.90 (s, 3H), 3.23.4 (m, 2H), 3.13.2 (m, 1H), 2.72.8 (m, 1H), 1.9 2.0 (m, 2H), 1.21 (s, 9H), 0.8-0.9 (m, 3H).

### Step D: 2-((2-(1-((tert-Butoxycarbonyl)(2-(6-methoxy-3-nitropyridin-2-yl)ethyl)amino)-propyl)-4-fluorophenyl)amino)-5-fluoro-4-(trifluoromethyl)benzoic acid 126d

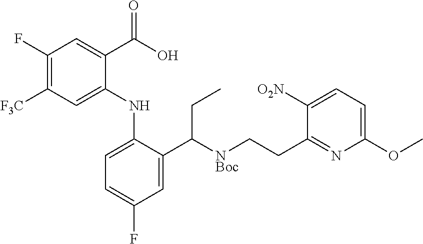 [SMILES: CCC(c1cc(F)ccc1Nc1cc(C(F)(F)F)c(F)cc1C(=O)O)N(CCc1nc(OC)ccc1[N+](=O)[O-])C(=O)OC(C)(C)C]

Following the procedure outlined in Example 41, Step C, stirring the reaction mixture at 50° C. for 20 h, 2-((2-(1-((tert-butoxycarbonyl)(2-(6-methoxy-3-nitropyridin-2-yl)ethyl)amino)-propyl)-4-fluorophenyl)amino)-5-fluoro-4-(trifluoromethyl)benzoic acid 126d (2.10 g, 99% yield) was prepared as a light yellow solid. HPLC/MS 1.19 min (B), [M+H]⁺ 655.2. ¹H NMR (DMSO-d₆, 400 MHz): δ 13.57 (s, 1H), 9.15 (br s, 1H), 8.26 (d, 1H, J=8.8 Hz), 7.79 (br d, 1H, J=9.8 Hz), 7.44 (dd, 1H, J=2.9, 10.3 Hz), 7.37 (dd, 1H, J=5.9, 8.8 Hz), 7.24 (dt, 1H, J=2.7, 8.2 Hz), 6.84 (d, 1H, J=8.8 Hz), 6.54 (br s, 1H), 5.30 (br s, 1H), 3.91 (s, 3H), 3.2-3.3 (m, 2H), 3.1-3.2 (m, 1H), 2.6-2.7 (m, 1H), 1.9-2.0 (m, 2H), 1.20 (s, 9H), 0.82 (t, 3H, J=7.3 Hz).

### Step E: 2-((2-(1-((2-(3-Amino-6-methoxypyridin-2-yl)ethyl)(tert-butoxycarbonyl)-amino)propyl)-4-fluorophenyl)amino)-5-fluoro-4-(trifluoromethyl)benzoic acid 126e

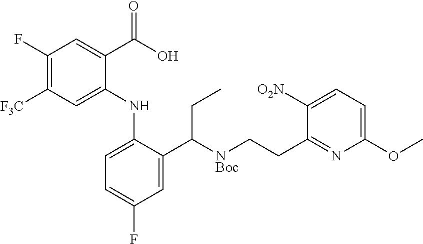 [SMILES: CCC(c1cc(F)ccc1Nc1cc(C(F)(F)F)c(F)cc1C(=O)O)N(CCc1nc(OC)ccc1[N+](=O)[O-])C(=O)OC(C)(C)C]

Following the procedure outlined in Example 41, Step D, 2-((2-(1-((2-(3-amino-6-methoxypyridin-2-yl)ethyl)(tert-butoxycarbonyl)amino)propyl)-4-fluorophenyl)amino)-5-fluoro-4-(trifluoromethyl)benzoic acid 126e (2.10 g, 100% yield) was prepared as a light brown solid. HPLC/MS 1.11 min (B), [M+H]⁺ 625.2. ¹H NMR (DMSO-d₆, 400 MHz): δ 9.0-9.4 (m, 1H), 7.81 (br s, 1H), 7.49 (dd, 1H, J=2.9, 9.8 Hz), 7.39 (dd, 1H, J=5.9, 8.8 Hz), 7.27 (dt, 1H, J=2.9, 8.3 Hz), 6.98 (d, 1H, J=8.3 Hz), 6.55 (br s, 1H), 6.39 (d, 1H, J=8.3 Hz), 5.1-5.4 (m, 1H), 3.9-4.3 (m, 2H), 3.66 (s, 3H), 3.3-3.6 (m, 1H), 3.0-3.2 (m, 2H), 2.6-2.7 (m, 1H), 1.99 (s, 1H), 1.8-1.9 (m, 2H), 1.2-1.3 (m, 9H), 0.8-0.9 (m, 3H).

### Step F: tert-Butyl 10-ethyl-2,8-difluoro-15-methoxy-19-oxo-3-(trifluoromethyl)-5,10,12,13,18,19-hexahydro-11H-dibenzo[c,f]pyrido[3,2-j][1,5,9]triazacyclotridecine-11-carboxylate 126f

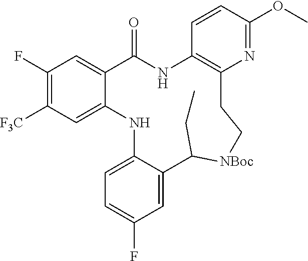 [SMILES: CCC1c2cc(F)ccc2Nc2cc(C(F)(F)F)c(F)cc2C(=O)Nc2ccc(OC)nc2CCN1C(=O)OC(C)(C)C]

Following the procedure outlined in Example 44, Step E, stirring the reaction mixture at room temperature for 4 h, tert-butyl 10-ethyl-2,8-difluoro-15-methoxy-19-oxo-3-(trifluoro-methyl)-5,10,12,13,18,19-hexahydro-11H-dibenzo[c,f]pyrido[3,2-j][1,5,9]triazacyclo-tridecine-11-carboxylate 126f (1.07 g, 55% yield) was prepared as an off-white solid. HPLC/MS 1.55 min (B), [M+H]⁺ 607.2. ¹H NMR (DMSO-d₆, 400 MHz): δ 10.81 (br s, 1H), 8.08 (br d, 1H, J=11.7 Hz), 7.6-7.9 (m, 3H), 7.44 (dd, 1H, J=2.7, 10.0 Hz), 7.03 (br s, 1H), 6.69 (br d, 1H, J=8.3 Hz), 6.35 (br s, 1H), 5.66 (br s, 1H), 3.79 (s, 3H), 3.7-3.8 (m, 1H), 3.2-3.3 (m, 1H), 1.9-2.2 (m, 3H), 1.5-1.7 (m, 1H), 0.9-1.4 (m, 9H), 0.88 (br t, 3H, J=7.1 Hz).

### Step G: tert-Butyl 10-ethyl-2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 126g

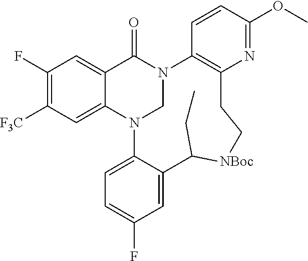 [SMILES: CCC1c2cc(F)ccc2N2CN(C(=O)c3cc(F)c(C(F)(F)F)cc32)c2ccc(OC)nc2CCN1C(=O)OC(C)(C)C]

Following the procedure outlined in Example 41, Step F, stirring the reaction at 90° C. for 68 h, tert-butyl 10-ethyl-2,8-difluoro-15-methoxy-19-oxo-3-(trifluoromethyl)-12,13-dihydro-19H-5,18-methanodibenzo[c,f]pyrido[3,2-j][1,5,9]triazacyclotridecine-11(10H)-carboxylate 126g (1.01 g, 93% yield) was prepared as a light yellow solid. HPLC/MS 1.54 min (B), [M+H]⁺ 619.2. ¹H NMR (DMSO-d₆, 400 MHz): δ 7.92 (d, 1H, J=10.3 Hz), 7.72 (d, 1H, J=8.3 Hz), 7.62 (dd, 1H, J=2.9, 10.3 Hz), 7.53 (dd, 1H, J=5.6, 8.6 Hz), 7.35 (dt, 1H, J=2.9, 8.3 Hz), 6.79 (d, 1H, J=8.8 Hz), 6.49 (d, 1H, J=5.4 Hz), 5.58 (d, 1H, J=11.2 Hz), 4.66 (br d, 1H, J=11.2 Hz), 4.22 (br t, 1H, J=7.6 Hz), 3.86 (s, 3H), 3.4-3.5 (m, 2H), 3.0-3.1 (m, 1H), 2.5-2.6 (m, 1H), 2.1-2.3 (m, 1H), 1.7-1.9 (m, 1H), 1.24 (s, 9H), 0.65 (t, 3H, J=7.1 Hz).

### Step H: 10-Ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 126

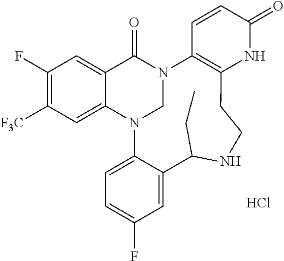 [SMILES: CCC1NCCc2[nH]c(=O)ccc2N2CN(c3cc(C(F)(F)F)c(F)cc3C2=O)c2ccc(F)cc21 ; Cl]

Following the procedure outlined in Example 41, Step G, stirring the reaction mixture at 90° C. for 17 h, 10-ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride (730 mg, 82% yield) was prepared as a white solid. HPLC/MS 1.02 min (B), [M+H]⁺ 505.2. ¹H NMR (DMSO-d₆, 400 MHz): δ 9.9-10.0 (m, 1H), 9.6-9.8 (m, 1H), 7.92 (d, 1H, J=10.3 Hz), 7.74 (dd, 1H, J=2.9, 9.8 Hz), 7.64 (dd, 1H, J=5.4, 8.8 Hz), 7.5-7.5 (m, 2H), 6.39 (d, 1H, J=5.9 Hz), 6.33 (d, 1H, J=9.3 Hz), 5.50 (d, 1H, J=11.7 Hz), 4.86 (d, 1H, J=11.7 Hz), 4.61 (br s, 1H), 3.1-3.3 (m, 2H), 2.80 (br s, 1H), 2.7-2.8 (m, 1H), 2.0-2.1 (m, 2H), 0.47 (t, 3H, J=7.1 Hz).

### Example 127 and Example 128

### Step A: (10R)-10-Ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione

### (10S)-10-Ethyl-2,8-difluoro-3-(trifluoromethyl)-10,11,12,13-tetrahydro-19H-5,18-methanodibenzo[c,f]pyrido[3,2-i][1,5,9]triazacyclotridecine-15,19(14H)-dione

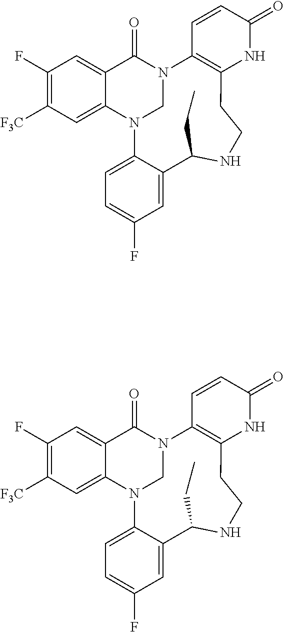 [SMILES: CC[C@H]1NCCc2[nH]c(=O)ccc2N2CN(c3cc(C(F)(F)F)c(F)cc3C2=O)c2ccc(F)cc21 ; CC[C@@H]1NCCc2[nH]c(=O)ccc2N2CN(c3cc(C(F)(F)F)c(F)cc3C2=O)c2ccc(F)cc21]

Racemic 10-ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione hydrochloride Example 126 (728 g, 1.44 mmol) was resolved using chiral chromatography (CC4 column, 150×4.6 mm, 5 μm), eluting with an isocratic gradient of 65:35 CO₂-MeOH to afford:

First-eluting stereoisomer (10R)-10-ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione Example 127 (316 mg, 43% yield). HPLC/MS 1.02 min (B), [M+H]⁺ 505.1. ¹H NMR (DMSO-d₆, 400 MHz): δ 11.90 (br s, 1H), 9.3-10.1 (m, 1H), 7.90 (d, 1H, J=10.3 Hz), 7.3-7.8 (m, 4H), 6.41 (br s, 1H), 6.28 (br s, 1H), 5.51 (d, 1H, J=11.2 Hz), 4.81 (br d, 1H, J=10.8 Hz), 4.4-4.7 (m, 1H), 2.5-3.3 (m, 4H), 1.8-2.4 (m, 2H), 0.46 (t, 3H, J=7.1 Hz).

Second-eluting stereoisomer (10S)-10-ethyl-2,8-difluoro-3-(trifluoromethyl)-10,11,12,13-tetrahydro-19H-5,18-methanodibenzo[c,f]pyrido[3,2-j][1,5,9]triazacyclotridecine-15,19(14H)-dione Example 128 (323 mg, 43% yield). HPLC/MS 1.02 min (B), [M+H]⁺ 505.1. ¹H NMR (DMSO-d₆, 400 MHz): δ 11.93 (br s, 1H), 9.5-10.1 (m, 1H), 7.9-8.0 (m, 1H), 7.3-7.8 (m, 4H), 6.40 (br d, 1H, J=4.9 Hz), 6.30 (br s, 1H), 5.50 (d, 1H, J=11.7 Hz), 4.82 (br d, 1H, J=11.2 Hz), 4.57 (br s, 1H), 3.43 (br s, 1H), 2.6-3.2 (m, 3H), 1.8-2.5 (m, 2H), 0.4-0.5 (m, 3H).

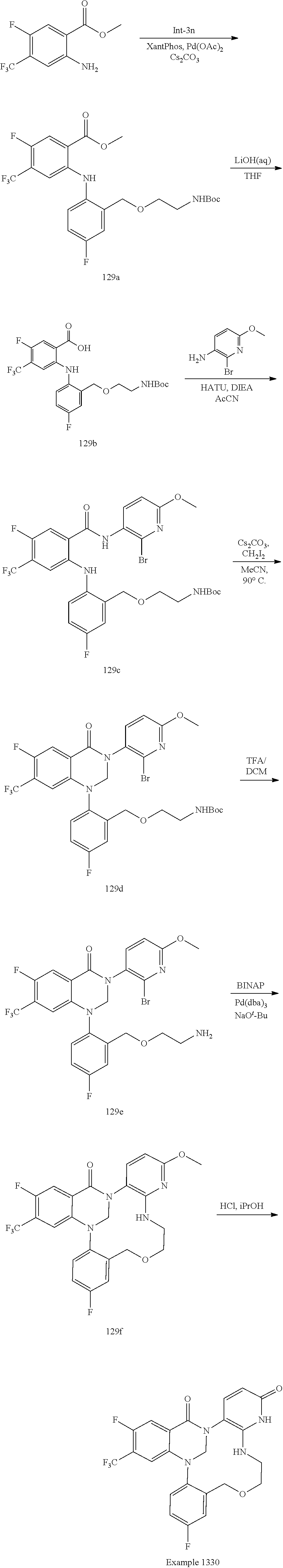

### Example 129

### 2⁶,3⁴-Difluoro-2′-(trifluoromethyl)-1¹,1²,2¹,2² 2³ 2⁴-hexahydro-5-oxa-8-aza-2(3,1)quinazolina-1(5,6)-pyridina-3(1,2′)-benzenacyclooctaphane-1²,2⁴-dione

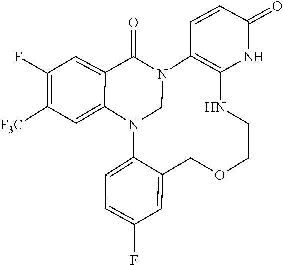 [SMILES: O=C1c2cc(F)c(C(F)(F)F)cc2N2CN1c1ccc(=O)[nH]c1NCCOCc1cc(F)ccc12]

### Step A: Methyl-(2-(2-((tert-butoxycarbonyl)amino)ethoxy)methyl)-4-fluorophenyl)-amino)-5-fluoro-4-(trifluoromethyl)benzoate 129a

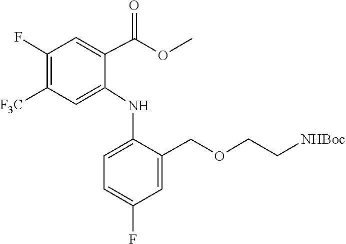 [SMILES: COC(=O)c1cc(F)c(C(F)(F)F)cc1Nc1ccc(F)cc1COCCNC(=O)OC(C)(C)C]

Following the preparation outlined in Example 7, Step A, substituting ethyl 2-bromo-5-(trifluoromethyl)benzoate with tert-butyl (2-((2-bromo-5-fluorobenzyl)oxy)ethyl)carbamate Int-3n, 2-(but-3-en-1-yl)-4-fluoroaniline Int-3a with methyl 2-amino-5-fluoro-4-(trifluoro-methyl)benzoate, BINAP with Xantphos, and stirring the reaction mixture at 120° C. for 16 h, methyl 2-((2-((2-((tert-butoxycarbonyl)amino)ethoxy)methyl)-4-fluorophenyl)amino)-5-fluoro-4-(trifluoromethyl)benzoate 129a (36.2 mg, 24% yield) was prepared as a yellow oil. HPLC/MS 1.46 min (A), [M+H]⁺ 505.2. ¹H NMR (CD₃OD, 400 MHz): δ 9.49 (s, 1H), 7.85 (d, J=11.2 Hz, 1H), 7.39 (dd, J=8.8, 5.4 Hz, 1H), 7.27 (dd, J=8.8, 2.9 Hz, 1H), 7.19 (d, J=5.9 Hz, 1H), 7.13 (td, J=8.6, 2.9 Hz, 1H), 4.49 (s, 2H), 3.99 (s, 3H), 3.51-3.58 (m, 2H), 3.32-3.35 (m, 2H), 1.42 (s, 9H).

### Step B: 2-((2-((2-((tert-Butoxycarbonyl)amino)ethoxy)methyl)-4-fluorophenyl)amino)-5-fluoro-4-(trifluoromethyl)benzoic acid 129b

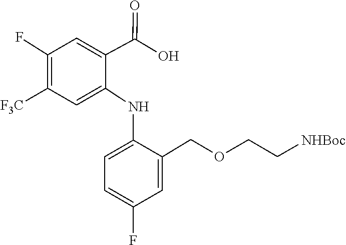 [SMILES: CC(C)(C)OC(=O)NCCOCc1cc(F)ccc1Nc1cc(C(F)(F)F)c(F)cc1C(=O)O]

Following the procedure outlined in Example 27, Step B, stirring the reaction mixture at RT for 3 h, 2-((2-((2-((tert-butoxycarbonyl)amino)ethoxy)methyl)-4-fluorophenyl)amino)-5-fluoro-4-(trifluoromethyl)benzoic acid 129b (1.08 g, 100% yield) was prepared as a yellow oil. HPLC/MS 1.36 min (A), [M+H]⁺ 491.1. ¹H NMR (CDCl₃, 400 MHz): δ 7.74-7.90 (m, 1H), 7.32-7.42 (m, 2H), 7.04-7.16 (m, 2H), 4.44 (s, 2H), 3.58 (br s, 2H), 3.37-3.46 (m, 2H), 1.47 (s, 9H).

### Step C: tert-Butyl (2-((2-((2-((2-bromo-6-methoxypyridin-3-yl)carbamoyl)-4-fluoro-5-(trifluoromethyl)phenyl)amino)-5-fluorobenzyl)oxy)ethyl)carbamate 129c

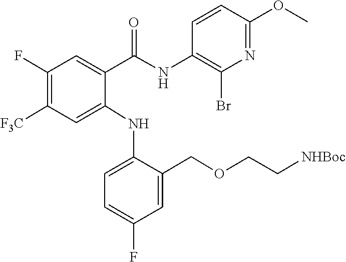 [SMILES: COc1ccc(NC(=O)c2cc(F)c(C(F)(F)F)cc2Nc2ccc(F)cc2COCCNC(=O)OC(C)(C)C)c(Br)n1]

Following the procedure outlined in Example 27, Step D, using 2-((2-((2-((tert-butoxycarbonyl)amino)ethoxy)methyl)-4-fluorophenyl)amino)-5-fluoro-4-(trifluoromethyl) benzoic acid 124b and 2-bromo-6-methoxypyridin-3-amine as substrates, substituting pyoxim with HATU and stirring the reaction mixture at room temperature for 22 h, tert-butyl (2-((2-((2-((2-bromo-6-methoxypyridin-3-yl)carbamoyl)-4-fluoro-5-(trifluoromethyl)phenyl)amino)-5-fluorobenzyl)oxy)ethyl)carbamate 129c (608 mg, 41% yield) was prepared as a yellow solid. HPLC/MS 1.47 min (A), [M+H]⁺ 675.0. ¹H NMR (CDCl₃, 400 MHz): δ 9.22 (br s, 1H), 8.49 (br d, J=8.3 Hz, 1H), 8.14 (br s, 1H), 7.50 (d, J=10.3 Hz, 1H), 7.37 (d, J=5.9 Hz, 1H), 7.27-7.32 (m, 2H), 7.11 (dd, J=8.8, 2.9 Hz, 1H), 7.06 (td, J=8.4, 3.2 Hz, 1H), 6.81 (d, J=8.8 Hz, 1H), 4.51 (s, 2H), 3.97 (s, 3H), 3.60 (t, J=4.9 Hz, 2H), 3.41 (br d, J=4.9 Hz, 2H), 1.40 (s, 9H).

### Step D: tert-Butyl (2-((2-(3-(2-bromo-6-methoxypyridin-3-yl)-6-fluoro-4-oxo-7-(trifluoromethyl)-3,4-dihydroquinazolin-1(2H)-yl)-5-fluorobenzyl)oxy)ethyl)carbamate 129d

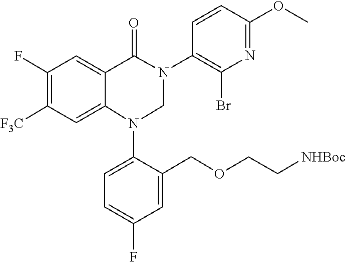 [SMILES: COc1ccc(N2CN(c3ccc(F)cc3COCCNC(=O)OC(C)(C)C)c3cc(C(F)(F)F)c(F)cc3C2=O)c(Br)n1]

Following the procedure outlined in Example 27, Step E, stirring the reaction mixture at 80° C. for 2 days, tert-butyl (2-((2-(3-(2-bromo-6-methoxypyridin-3-yl)-6-fluoro-4-oxo-7-(trifluoromethyl)-3,4-dihydroquinazolin-1(2H)-yl)-5-fluorobenzyl)oxy)ethyl)carbamate 129d (320 mg, 52% yield) was prepared as a light yellow solid. HPLC/MS 1.39 min (A), [M+H]⁺ 687.1. ¹H NMR (CDCl₃, 400 MHz): δ 7.95 (d, J=9.8 Hz, 1H), 7.44-7.63 (m, 1H), 7.38 (br d, J=8.3 Hz, 1H), 7.14 (br d, J=4.9 Hz, 2H), 6.79 (d, J=8.3 Hz, 1H), 6.59 (d, J=5.9 Hz, 1H), 5.14-5.31 (m, 1H), 4.92-5.08 (m, 1H), 4.69-4.83 (m, 1H), 4.54 (br d, J=1.5 Hz, 1H), 3.97 (s, 3H), 3.56 (br t, J=5.1 Hz, 2H), 3.32 (br s, 2H), 1.46 (s, 9H).

### Step E: 1-(2-((2-Aminoethoxy)methyl)-4-fluorophenyl)-3-(2-bromo-6-methoxypyridin-3-yl)-6-fluoro-7-(trifluoromethyl)-2,3-dihydroquinazolin-4(11H)-one 129e

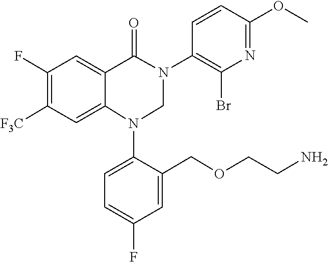 [SMILES: COc1ccc(N2CN(c3ccc(F)cc3COCCN)c3cc(C(F)(F)F)c(F)cc3C2=O)c(Br)n1]

To a solution of tert-butyl (2-((2-(3-(2-bromo-6-methoxypyridin-3-yl)-6-fluoro-4-oxo-7-(trifluoromethyl)-3,4-dihydroquinazolin-1(2H)-yl)-5-fluorobenzyl)oxy)ethyl)carbamate 129d (317 mg, 461 μmol) in DCM (4.5 mL) was added TFA (355 μL, 4.61 mmol) and the reaction mixture was stirred at RT for 18 h. The reaction mixture was quenched by with aq. sat'd NaHCO₃ and extracted with DCM (3×). The combined organics extracts were washed with brine, dried over MgSO₄ and concentrated under reduced pressure to afford 1-(2-((2-aminoethoxy)methyl)-4-fluorophenyl)-3-(2-bromo-6-methoxypyridin-3-yl)-6-fluoro-7-(trifluoro-methyl)-2,3-dihydroquinazolin-4(1H)-one 129e (240 mg, 84% yield) as a yellow oil. HPLC/MS 0.89 min (A), [M+H]⁺ 587.0. ¹H NMR (CD₂Cl₂, 400 MHz): δ 7.93 (d, J=10.3 Hz, 1H), 7.52-7.68 (m, 1H), 7.42 (br d, J=1.5 Hz, 1H), 7.19-7.35 (m, 1H), 7.12-7.18 (m, 1H), 6.83 (d, J=8.3 Hz, 1H), 6.64 (br s, 1H), 5.26 (br t, J=16.6 Hz, 1H), 5.02 (br d, J=2.9 Hz, 1H), 4.64-4.80 (m, 1H), 4.40-4.55 (m, 1H), 3.97 (s, 3H), 3.52 (t, J=5.4 Hz, 2H), 2.83 (t, J=5.4 Hz, 2H).

### Step F: 2⁶,3⁴-Difluoro-1⁶-methoxy-2⁷-(trifluoromethyl)-2¹,2²,2³ 2⁴-tetrahydro-5-oxa-8-aza-2(3,1)-quinazolina-1(3,2)-pyridina-3(1,2)-benzenacyclooctaphan-2⁴-one 129f

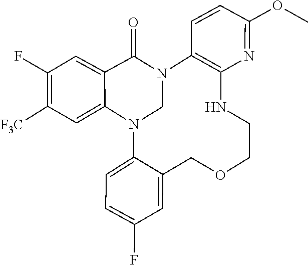 [SMILES: COc1ccc2c(n1)NCCOCc1cc(F)ccc1N1CN2C(=O)c2cc(F)c(C(F)(F)F)cc21]

To 1-(2-((2-aminoethoxy)methyl)-4-fluorophenyl)-3-(2-bromo-6-methoxypyridin-3-yl)-6-fluoro-7-(trifluoromethyl)-2,3-dihydroquinazolin-4(1H)-one 129e (218 mg, 371 μmol) in toluene (8.0 mL) was added BINAP (46.2 mg, 74.2 μmol) followed by tris(dibezylideneacetone)dipalladium (34.0 mg, 37.1 μmol) and sodium tert-butoxide (178 mg, 1.86 mmol) and the reaction mixture was stirred at 100° C. for 4 h. The reaction mixture was cooled to ambient temperature, filtered through celite pad and washed with copious EtOAc. The filtrate was concentrated under reduced pressure and purified by silica gel flash column chromatography (40 g), eluting with a 100% heptanes to 50% EtOAc-heptanes gradient. Product fractions were combined and evaporated under reduced pressure to afford 2⁶,3⁴-difluoro-1⁶-methoxy-2⁷-(trifluoromethyl)-2′,2²,2³,2⁴-tetrahydro-5-oxa-8-aza-2(3,1)-quinazolina-1(3,2)-pyridina-3(1,2)-benzenacyclooctaphan-2⁴-one 129f (72.4 mg, 37% yield) as a yellow solid. HPLC/MS 1.21 min (A), [M+H]⁺ 507.1. ¹H NMR (CD₃OD, 400 MHz): δ 7.89 (d, J=10.3 Hz, 1H), 7.50 (dd, J=8.8, 5.4 Hz, 1H), 7.45 (d, J=8.3 Hz, 1H), 7.41-7.44 (m, 1H), 7.32 (td, J=8.4, 3.2 Hz, 1H), 6.50 (d, J=5.4 Hz, 1H), 6.10 (d, J=8.3 Hz, 1H), 5.37 (d, J=9.8 Hz, 1H), 4.82 (d, J=9.3 Hz, 1H), 4.49 (d, J=9.8 Hz, 1H), 4.38 (d, J=8.8 Hz, 1H), 4.03 (ddd, J=10.3, 5.1, 3.7 Hz, 1H), 3.87 (s, 3H), 3.67-3.72 (m, 1H), 3.65 (dd, J=7.6, 3.2 Hz, 1H), 3.58-3.62 (m, 1H), 3.41-3.49 (m, 1H).

### Step G: 2⁶,3⁴-Difluoro-2⁷-(trifluoromethyl)-1¹,1²,2¹,2²,2³,2⁴-hexahydro-5-oxa-8-aza-2(3,1)-quinazolina-1(5,6)-pyridina-3(1,2)-benzenacyclooctaphane-1²,2⁴-dione Example 129

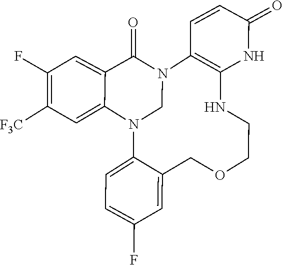 [SMILES: O=C1c2cc(F)c(C(F)(F)F)cc2N2CN1c1ccc(=O)[nH]c1NCCOCc1cc(F)ccc12]

Following the procedure outlined in Example 27, Step F, stirring the reaction mixture at 90° C. for 24 h, 2⁶,3⁴-difluoro-2⁷-(trifluoromethyl)-1¹,1²,2¹,2²,2³,2⁴-hexahydro-5-oxa-8-aza-2(3,1)-quinazolina-1(5,6)-pyridina-3(1,2)-benzenacyclooctaphane-1²,2⁴-dione (10.2 mg, 14% yield) was prepared. HPLC/MS 0.96 min (A), [M+H]⁺ 493.1. ¹H NMR (CD₂Cl₂, 400 MHz): δ 7.86 (d, J=10.3 Hz, 1H), 7.20-7.36 (m, 4H), 6.46 (d, J=5.9 Hz, 1H), 5.81 (d, J=8.8 Hz, 1H), 5.19-5.23 (m, 2H), 4.70 (d, J=9.3 Hz, 1H), 4.44 (d, J=9.3 Hz, 1H), 4.33 (d, J=8.8 Hz, 1H), 4.02-4.07 (m, 1H), 3.58-3.66 (m, 2H), 3.44-3.51 (m, 1H).

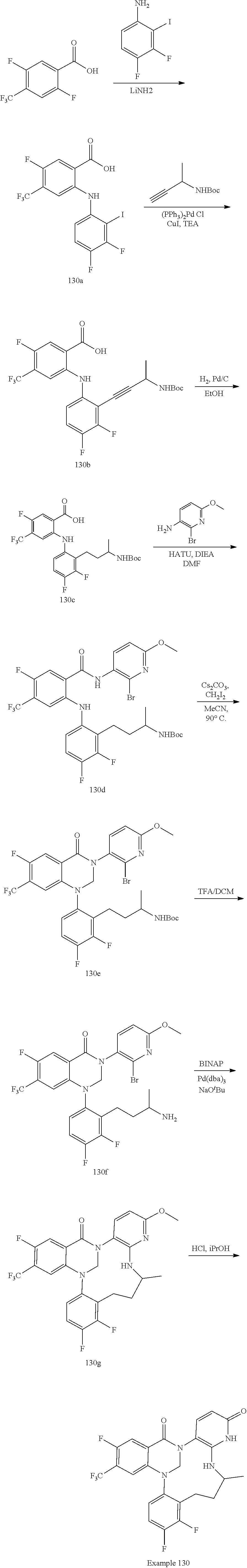

### Example 130

### 2,8,9-Trifluoro-12-methyl-3-(trifluoromethyl)-10,11,12,13-tetrahydro-19H-5,18-methano-dibenzor[f,i]pyridor[2,3-b][1,4,8]triazacyclotridecine-15.19(14H)-dione

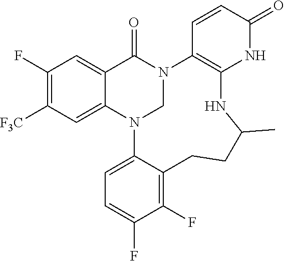 [SMILES: CC1CCc2c(ccc(F)c2F)N2CN(C(=O)c3cc(F)c(C(F)(F)F)cc32)c2ccc(=O)[nH]c2N1]

### Step A: 2-((3,4-Difluoro-2-iodophenyl)amino)-5-fluoro-4-(trifluoromethyl)benzoic acid 130a

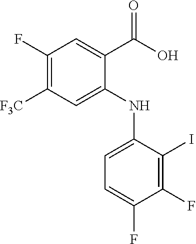 [SMILES: O=C(O)c1cc(F)c(C(F)(F)F)cc1Nc1ccc(F)c(F)c1I]

To 2,5-difluoro-4-(trifluoromethyl)benzoic acid (2.00 g, 8.85 mmol) and 3,4-difluoro-2-iodoaniline (2.48 g, 9.73 mmol) in THF (35.4 mL) was added lithium amide (710.8 mg, 30.9 mmol) and the reaction mixture was heated at 50° C. for 13 h. The reaction mixture was cooled to ambient temperature, diluted with EtOAc, washed with 1N HCl and brine, dried over Na₂SO₄, filtered, concentrated under reduced pressure, loaded onto silica gel and purified by silica gel flash column chromatography eluting with a 100% heptanes to 25% 3:1 EtOAc:EtOH-heptanes gradient. Product fractions were combined, evaporated under reduced pressure and recrystallized from DCM to afford 2-((3,4-difluoro-2-iodophenyl)amino)-5-fluoro-4-(trifluoromethyl)benzoic acid 130a (1.94 g, 43% yield) as a beige solid. HPLC/MS 1.34 min (A), [M−H]⁺ 460. ¹H NMR (DMSO-d₆, 400 MHz): δ 7.09 (d, J=6.36 Hz, 1H), 7.28-7.44 (m, 1H), 7.44-7.63 (m, 1H), 7.91 (d, J=11.25 Hz, 1H), 9.54 (br s, 1H), 14.01-14.26 (m, 1H).

### Step B: 2-((2-(3-((tert-Butoxycarbonyl)amino)but-1-yn-1-yl)-3,4-difluorophenyl)amino)-5-fluoro-4-(trifluoromethyl)benzoic acid 130b

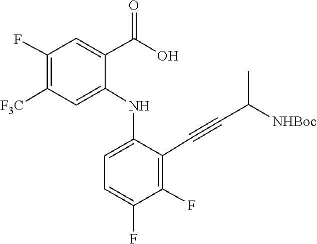 [SMILES: CC(C#Cc1c(Nc2cc(C(F)(F)F)c(F)cc2C(=O)O)ccc(F)c1F)NC(=O)OC(C)(C)C]

Following the preparation outlined in Int-3h, Step A, substituting 3,4-difluoro-2-iodoaniline with 2-((3,4-difluoro-2-iodophenyl)amino)-5-fluoro-4-(trifluoromethyl)benzoic acid 130a, tert-butyl prop-2-yn-1-ylcarbamate with tert-butyl but-3-yn-2-ylcarbamate, and stirring the reaction at 80° C. for 2 h, 2-((2-(3-((tert-butoxycarbonyl)amino)but-1-yn-1-yl)-3,4-difluoro-phenyl)amino)-5-fluoro-4-(trifluoromethyl)benzoic acid 130b (953 mg, 82% yield) was prepared. HPLC/MS 1.43 min (A), [M−H]⁺ 501. ¹H NMR (DMSO-d₆, 400 MHz): δ 1.33 (d, J=6.85 Hz, 3H), 1.37 (s, 9H), 4.52-4.64 (m, 1H), 7.26-7.38 (m, 1H), 7.39-7.52 (m, 2H), 7.80-8.04 (m, 1H), 9.77-9.91 (m, 1H), 14.01-14.29 (m, 1H).

### Step C: 2-((2-(3-((tert-Butoxycarbonyl)amino)butyl)-3,4-difluorophenyl)amino)-5-fluoro-4-(trifluoromethyl)benzoic acid 130c

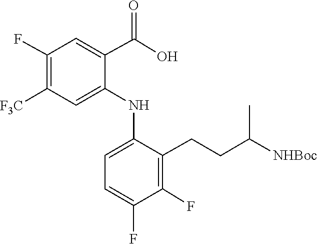 [SMILES: CC(CCc1c(Nc2cc(C(F)(F)F)c(F)cc2C(=O)O)ccc(F)c1F)NC(=O)OC(C)(C)C]

Following the procedure outlined in Example 27, Step C, stirring at RT for 16 h, 2-((2-(3-((tert-butoxycarbonyl)amino)butyl)-3,4-difluorophenyl)amino)-5-fluoro-4-(trifluoromethyl)-benzoic acid 130c (635 g, 63% yield) was prepared. HPLC/MS 1.41 min (A), [M−H]⁺ 505. ¹H NMR (DMSO-d₆, 400 MHz): δ 1.22-1.41 (m, 12H), 1.47-1.61 (m, 2H), 2.54-2.66 (m, 2H), 3.35-3.47 (m, 1H), 6.65 (br d, J=9.29 Hz, 1H), 6.93 (br d, J=5.38 Hz, 1H), 7.22 (br dd, J=8.80, 2.93 Hz, 1H), 7.35 (q, J=9.29 Hz, 1H), 7.87 (d, J=10.76 Hz, 1H), 9.34-9.56 (m, 1H), 13.87-14.13 (m, 1H).

### Step D: tert-Butyl (2-((2-((2-((2-bromo-6-methoxypyridin-3-yl)carbamoyl)-4-fluoro-5-(trifluoromethyl)phenyl)amino)-5-fluorobenzyloxy)ethyl)carbamate 130d

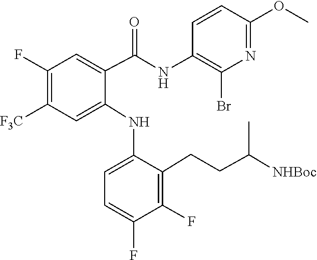 [SMILES: COc1ccc(NC(=O)c2cc(F)c(C(F)(F)F)cc2Nc2ccc(F)c(F)c2CCC(C)NC(=O)OC(C)(C)C)c(Br)n1]

Following the procedure outlined in Example 27, Step D, using 2-((2-(3-((tert-butoxycarbonyl)amino)butyl)-3,4-difluorophenyl)amino)-5-fluoro-4-(trifluoromethyl)benzoic acid 130c and 2-bromo-6-methoxypyridin-3-amine as substrates, substituting Pyoxim with HATU and stirring the reaction mixture at room temperature for 20 h, tert-butyl (2-((2-((2-((2-bromo-6-methoxypyridin-3-yl)carbamoyl)-4-fluoro-5-(trifluoromethyl)phenyl)amino)-5-fluorobenzyl)-oxy)ethyl)carbamate 130d (451 mg, 52% yield) was prepared. HPLC/MS 1.52 min (A), [M+H]⁺ 693. ¹H NMR (CD₃OD, 400 MHz): δ 1.08 (d, J=6.85 Hz, 3H), 1.38 (s, 9H), 1.63 (br s, 2H), 2.66-2.76 (m, 2H), 3.49-3.65 (m, 1H), 3.96 (s, 3H), 6.33-6.43 (m, 1H), 6.84-6.92 (m, 1H), 7.02-7.24 (m, 3H), 7.79-7.93 (m, 2H).

### Step E: tert-Butyl (4-(6-(3-(2-bromo-6-methoxypyridin-3-yl)-6-fluoro-4-oxo-7-(trifluoro-methyl)-3,4-dihydroquinazolin-1(2H)-yl)-2,3-difluorophenyl)butan-2-yl)carbamate 130e

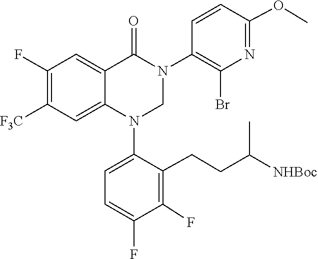 [SMILES: COc1ccc(N2CN(c3ccc(F)c(F)c3CCC(C)NC(=O)OC(C)(C)C)c3cc(C(F)(F)F)c(F)cc3C2=O)c(Br)n1]

Following the procedure outlined in Example 27, Step E, stirring the reaction mixture at 80° C. for 22 h, tert-butyl (4-(6-(3-(2-bromo-6-methoxypyridin-3-yl)-6-fluoro-4-oxo-7-(trifluoromethyl)-3,4-dihydroquinazolin-1(2H)-yl)-2,3-difluorophenyl)butan-2-yl)carbamate (223 mg, 46% yield) was prepared as a light brown solid. HPLC/MS 1.45 min (A), [M+H]⁺ 703, 705. ¹H NMR (DMSO-d₆, 400 MHz): δ 7.91 (br dd, J=9.3, 4.4 Hz, 1H), 7.84 (br d, J=7.8 Hz, 1H), 7.38-7.52 (m, 1H), 7.28 (br d, J=4.4 Hz, 1H), 6.98 (br dd, J=8.6, 3.7 Hz, 1H), 6.60-6.79 (m, 2H), 6.44-6.54 (m, 1H), 5.49-5.59 (m, 1H), 5.20-5.27 (m, 1H), 4.83-4.93 (m, 1H), 3.88 (s, 3H), 2.57-2.72 (m, 2H), 1.51-1.68 (m, 2H), 1.32 (br s, 9H), 0.87-1.01 (m, 3H).

### Step F: 1-(2-(3-Aminobutyl)-3,4-difluorophenyl)-3-(2-bromo-6-methoxypyridin-3-yl)-6-fluoro-7-(trifluoromethyl)-2,3-dihydroquinazolin-4(1H)-one 130f

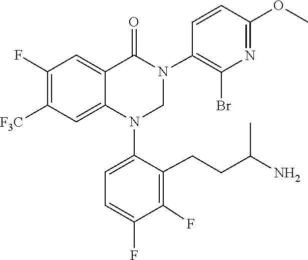 [SMILES: COc1ccc(N2CN(c3ccc(F)c(F)c3CCC(C)N)c3cc(C(F)(F)F)c(F)cc3C2=O)c(Br)n1]

Following the procedure outlined in Example 129, Step E, stirring the reaction at rt for 17 h, 1-(2-(3-aminobutyl)-3,4-difluorophenyl)-3-(2-bromo-6-methoxypyridin-3-yl)-6-fluoro-7-(trifluoromethyl)-2,3-dihydroquinazolin-4(1H)-one 130f (192 mg, 97% yield) was prepared as a brown solid, which was used without further purification. HPLC/MS 0.92 min (A), [M+H]⁺ 605.

### Step G: 2,8,9-Trifluoro-15-methoxy-12-methyl-3-(trifluoromethyl)-10,11,12,13-tetra-hydro-19H-5,18-methanodibenzo[f,i]pyrido[2,3-b][1,4,8]triazacyclotridecin-19-one 1302

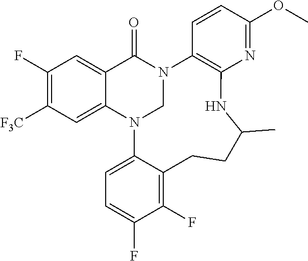 [SMILES: COc1ccc2c(n1)NC(C)CCc1c(ccc(F)c1F)N1CN2C(=O)c2cc(F)c(C(F)(F)F)cc21]

Following the procedure outlined in Example 129, Step F, stirring the reaction at 100° C. for 2 h, 2,8,9-trifluoro-15-methoxy-12-methyl-3-(trifluoromethyl)-10,11,12,13-tetrahydro-19H-5,18-methanodibenzo[f,i]pyrido[2,3-b][1,4,8]triazacyclotridecin-19-one 130g (53.0 mg, 33% yield) was prepared as a yellow solid. HPLC/MS 1.31/1.36 min (atropisomers) (A), [M+H]⁺ 523.0. ¹H NMR (CDCl₃, 400 MHz): δ 1.06 (br d, J=6.36 Hz, 1H), 1.32 (d, J=6.85 Hz, 5H), 1.52-1.70 (m, 4H), 1.75-1.90 (m, 1H), 2.05 (dt, J=14.55, 7.15 Hz, 1H), 2.30-2.46 (m, 2H), 2.68-2.89 (m, 2H), 2.91-3.15 (m, 2H), 3.80-4.00 (m, 5H), 4.27 (d, J=10.76 Hz, 2H), 4.50 (br s, 1H), 4.58 (d, J=10.76 Hz, 1H), 5.26 (d, J=10.76 Hz, 2H), 5.45-5.69 (m, 2H), 6.04-6.23 (m, 2H), 6.52-6.58 (m, 1H), 6.61 (d, J=5.38 Hz, 1H), 6.93-7.15 (m, 2H), 7.17-7.29 (m, 2H), 7.39-7.51 (m, 2H), 7.89-8.11 (m, 1H).

### Step H: 2,8,9-Trifluoro-12-methyl-3-(trifluoromethyl)-10,11,12,13-tetrahydro-19H-5,18-methanodibenzo[f,i]pyrido[2,3-b][1,4,8]triazacyclotridecin-15,19(14H)-dione Example 130

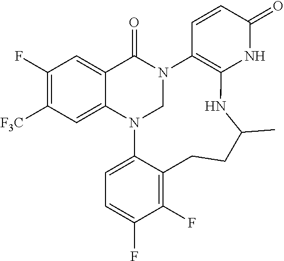 [SMILES: CC1CCc2c(ccc(F)c2F)N2CN(C(=O)c3cc(F)c(C(F)(F)F)cc32)c2ccc(=O)[nH]c2N1]

Following the procedure outlined in Example 27, Step F, stirring the reaction mixture at 90° C. for 44 h, 2,8,9-trifluoro-12-methyl-3-(trifluoromethyl)-10,11,12,13-tetrahydro-19H-5,18-methanodibenzo[f,i]pyrido[2,3-b][1,4,8]triazacyclotridecin-15,19(14H)-dione (23.2 mg, 43% yield) was prepared as an off white solid. HPLC/MS 1.11 min (A), [M+H]⁺ 509.2. ¹H NMR (CDCl₃, 400 MHz): δ 1.18-1.41 (m, 5H), 1.45-1.73 (m, 2H), 1.92 (br d, J=8.80 Hz, 2H), 2.23-2.41 (m, 1H), 2.66 (s, 1H), 2.97 (br s, 2H), 3.41-3.63 (m, 1H), 4.14-4.30 (m, 1H), 4.35 (s, 1H), 4.65 (d, J=10.76 Hz, 1H), 4.96-5.15 (m, 1H), 5.35 (d, J=10.76 Hz, 1H), 5.63 (d, J=11.25 Hz, 1H), 5.89 (br d, J=9.29 Hz, 1H), 6.08 (br d, J=9.29 Hz, 1H), 6.12-6.28 (m, 1H), 6.55 (d, J=5.38 Hz, 1H), 6.67 (d, J=5.38 Hz, 1H), 7.05-7.18 (m, 1H), 7.19-7.30 (m, 2H), 7.40 (d, J=9.29 Hz, 1H), 7.85-8.07 (m, 1H).

### Example 131

### 2,8,9-Trifluoro-10-methyl-3-(trifluoromethyl)-10,11,12,13-tetrahydro-19H-5,18-methano-dibenzo[f,i]pyrido[2,3-b][1,4,8]triazacyclotridecine-15,19(14H)-dione

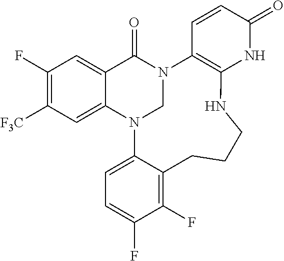 [SMILES: O=C1c2cc(F)c(C(F)(F)F)cc2N2CN1c1ccc(=O)[nH]c1NCCCc1c2ccc(F)c1F]

### Step A: tert-Butyl buta-2,3-dien-1-ylcarbamate acid 131a

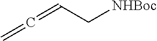

A mixture of tert-butyl prop-2-yn-1-ylcarbamate (8.00 g, 51.5 mmol), paraformaldehyde (3.87 g, 129 mmol), and cuprous iodide (4.91 g, 25.8 mmol) in dioxane (206 mL) was sparged for 5 minutes with nitrogen. Diisopropylamine (10.4 g, 103 mmol) was added, and the reaction mixture was refluxed for 22 h, cooled to ambient temperature, diluted with EtOAc, washed with aq. sat'd NH₄Cl solution (2×), brine, dried over Na₂SO₄, filtered, concentrated under reduced pressure and purified by silica gel flash column chromatography (120 g), eluting with a 20% EtOAc-hexane gradient. Product fractions were combined and evaporated under reduced pressure to afford tert-butyl buta-2,3-dien-1-ylcarbamate acid 131a (7.12 g, 81% yield). HPLC/MS 0.82 min (A), [M-^{t}Bu+H]⁺ 114.0. ¹H NMR (CDCl₃, 400 MHz): δ 1.49 (s, 9H), 3.77 (br s, 2H), 4.66 (dt, J=6.48, 3.36 Hz, 1H), 4.88 (dt, J=6.72, 3.24 Hz, 2H), 5.25 (t, J=6.36 Hz, 1H).

### Step B: tert-Butyl (3-(4,4,5,5-tetramethyl-1,3,2-dioxaborolan-2-yl)but-3-en-1-yl)carbamate 131b

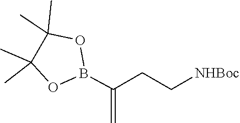 [SMILES: C=C(CCNC(=O)OC(C)(C)C)B1OC(C)(C)C(C)(C)O1]

A flask of 1,3-bis(2,6-diisopropylphenyl)imidazoliumchloride (894 mg, 2.10 mmol), cuprous chloride (208 mg, 2.10 mmol), and sodium 2-methylpropan-2-olate (1.62 g, 16.8 mmol) was purged with nitrogen. THF (120 mL) was added, the reaction vigorously sparged with N₂ for 10 min, then stirred for 30 min. A degassed solution of bis(pinacolato)diborane (11.8 g, 46.3 mmol) in THF (40 mL) was added, the reaction mixture stirred for 30 min, to which was added tert-butyl buta-2,3-dien-1-ylcarbamate 131a (7.12 g, 42.1 mmol) in a degassed solution of THF (45 mL). Degassed MeOH (10.2 mL, 252 mmol) was added, the reaction mixture stirred for 8 h at 22° C., then quenched by passing through a short plug of celite and eluting with EtOAc. The filtrate was concentrated under reduced pressure and purified by silica gel flash column chromatography, eluting with a 30% EtOAc-hexanes gradient. Product fractions were combined and evaporated under reduced pressure to afford tert-butyl (3-(4,4,5,5-tetramethyl-1,3,2-dioxaborolan-2-yl)but-3-en-1-yl)carbamate 131b (7.46 g, 57% yield). HPLC/MS 1.18 min (A), [M-^{t}Bu+H]⁺ 242.1. ¹H NMR (CDCl₃, 400 MHz): δ 1.25-1.35 (m, 13H), 1.41-1.50 (m, 10H), 2.35 (t, J=6.60 Hz, 2H), 3.24 (br d, J=5.87 Hz, 2H), 4.74-4.92 (m, 1H), 5.71 (br s, 1H), 5.81-6.00 (m, 1H), 6.21-6.29 (m, 1H).

### Step C: 2-((2-Bromo-3,4-difluorophenyl)amino)-5-fluoro-4-(trifluoromethyl)benzoic acid 131c

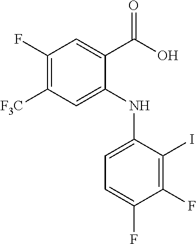 [SMILES: O=C(O)c1cc(F)c(C(F)(F)F)cc1Nc1ccc(F)c(F)c1I]

Following the procedure outlined in Example 130, Step A, substituting 3,4-difluoro-2-iodoaniline with 2-bromo-3,4-difluoroaniline, 2-((3,4-difluoro-2-iodophenyl)amino)-5-fluoro-4-(trifluoromethyl)benzoic acid 131c (1.86 g, 49% yield) was prepared as a beige solid. HPLC/MS 1.35 min (A), [M−H]⁺ 412.0. ¹H NMR (DMSO-d₆, 400 MHz): δ 13.5-14.8 (m, 1H), 9.60 (s, 1H), 7.90 (d, 1H, J=11.2 Hz), 7.5-7.6 (m, 1H), 7.42 (ddd, 1H, J=2.0, 4.5, 9.2 Hz), 7.21 (d, 1H, J=6.4 Hz).

### Step D: 2-((2-(4-((tert-Butoxycarbonyl)amino)but-1-en-2-yl)-3,4-difluorophenyl)amino)-5-fluoro-4-(trifluoromethyl)benzoic acid 131d

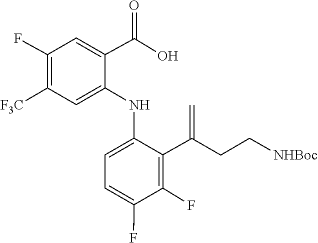 [SMILES: C=C(CCNC(=O)OC(C)(C)C)c1c(Nc2cc(C(F)(F)F)c(F)cc2C(=O)O)ccc(F)c1F]

A mixture of 2-((2-bromo-3,4-difluorophenyl)amino)-5-fluoro-4-(trifluoromethyl)benzoic acid 131c (650 mg, 1.57 mmol), tert-butyl (3-(4,4,5,5-tetramethyl-1,3,2-dioxaborolan-2-yl)but-3-en-1-yl)carbamate 131b (583 mg, 1.96 mmol), and XPhos Pd G3 (66.4 mg, 78.5 μmol) in dioxane (6.28 mL) and H₂O (1.57 mL) was sparged for 10 minutes with N₂. Potassium phosphate, tribasic (666 mg, 3.14 mmol) was added in a single portion, and the reaction mixture was heated at 80° C. for 18 h, the reaction cooled to ambient temperature, diluted with EtOAc, washed with aq. sat'd NH₄Cl, brine, dried over Na₂SO₄, filtered and the solvent evaporated under reduced pressure. The crude residue was purified by silica gel flash column chromagraphy, eluting with 100% heptanes to 30% EtOAc-heptanes gradient. Product fractions were combined, evaporated under reduced pressure and repurified by silica gel flash column chromatography, eluting with a 100% DCM to 15% MeOH-DCM gradient. Product fractions were combined and evaporated under reduced pressure to afford 2-((2-(4-((tert-butoxycarbonyl)amino)but-1-en-2-yl)-3,4-difluoro-phenyl)-amino)-5-fluoro-4-(trifluoromethyl)benzoic acid 131d (462 mg, 54% yield), containing about 14% of the internal olefin. This material was used without further purification. HPLC/MS 1.37 and 1.43 min (A), [M−H]⁺ 503.2.

### Step E: 2-((2-(4-((tert-Butoxycarbonyl)amino)butan-2-yl)-3,4-difluorophenyl)amino)-5-fluoro-4-(trifluoromethyl)benzoic acid 131e

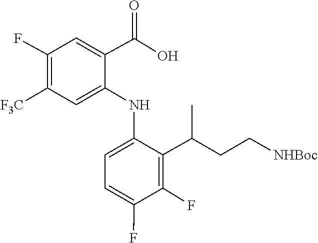 [SMILES: CC(CCNC(=O)OC(C)(C)C)c1c(Nc2cc(C(F)(F)F)c(F)cc2C(=O)O)ccc(F)c1F]

Following the procedure outlined in Example 27, Step C, stirring at room temperature for 18 h, 2-((2-(4-((tert-butoxycarbonyl)amino)butan-2-yl)-3,4-difluorophenyl)amino)-5-fluoro-4-(trifluoromethyl)benzoic acid 131e (265 mg, 53% yield) was prepared. HPLC/MS 1.40 min (A), [M−H]⁺ 505. ¹H NMR (DMSO-d₆, 400 MHz): δ 1.22-1.26 (m, 3H), 1.29 (s, 8H), 1.68-1.85 (m, 2H), 2.65-2.92 (m, 2H), 3.04-3.16 (m, 1H), 6.55-6.70 (m, 1H), 6.72-6.82 (m, 1H), 7.11-7.23 (m, 1H), 7.29-7.44 (m, 1H), 7.91 (s, 1H), 9.26-9.59 (m, 1H), 13.86-14.06 (m, 1H).

### Step F: tert-Butyl (3-(6-((2-((2-bromo-6-methoxypyridin-3-yl)carbamoyl)-4-fluoro-5-(trifluoromethyl)phenyl)amino)-2,3-difluorophenyl)butyl)carbamate 131f

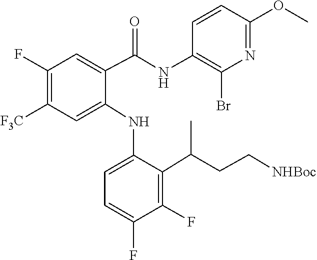 [SMILES: COc1ccc(NC(=O)c2cc(F)c(C(F)(F)F)cc2Nc2ccc(F)c(F)c2C(C)CCNC(=O)OC(C)(C)C)c(Br)n1]

Following the procedure outlined in Example 27, Step D, using 2-((2-(4-((tert-butoxycarbonyl)amino)butan-2-yl)-3,4-difluorophenyl)amino)-5-fluoro-4-(trifluoromethyl)-benzoic acid 131e and 2-bromo-6-methoxypyridin-3-amine as substrates, substituting pyoxim with HATU and stirring the reaction mixture at room temperature for 64 h, tert-butyl (3-(6-((2-((2-bromo-6-methoxypyridin-3-yl)carbamoyl)-4-fluoro-5-(trifluoromethyl)phenyl)amino)-2,3-difluorophenyl)butyl)carbamate 131f (232 mg, 62% yield) was prepared. HPLC/MS 1.51 min (A), [M+H]⁺ 691.0, 693.0. ¹H NMR (DMSO-d₆, 400 MHz): δ 1.23 (br d, J=6.85 Hz, 3H), 1.28 (s, 9H), 1.66-1.90 (m, 2H), 2.64-2.84 (m, 2H), 3.03-3.18 (m, 1H), 3.89 (s, 3H), 6.65 (br t, J=5.62 Hz, 1H), 6.87 (br d, J=5.87 Hz, 1H), 6.97 (d, J=8.80 Hz, 1H), 7.11-7.20 (m, 1H), 7.34 (q, J=9.29 Hz, 1H), 7.78-7.92 (m, 1H), 7.92-8.07 (m, 1H), 9.11 (br s, 1H), 10.45 (s, 1H).

### Step G: tert-Butyl (3-(6-(3-(2-bromo-6-methoxypyridin-3-yl)-6-fluoro-4-oxo-7-(trifluoromethyl)-3,4-dihydroquinazolin-1(2H)-yl)-2,3-difluorophenyl)butyl)carbamate 1312

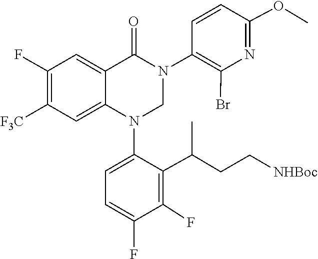 [SMILES: COc1ccc(N2CN(c3ccc(F)c(F)c3C(C)CCNC(=O)OC(C)(C)C)c3cc(C(F)(F)F)c(F)cc3C2=O)c(Br)n1]

Following the procedure outlined in Example 27, Step E, stirring the reaction mixture at 80° C. for 16 h, tert-butyl (3-(6-(3-(2-bromo-6-methoxypyridin-3-yl)-6-fluoro-4-oxo-7-(trifluoro-methyl)-3,4-dihydroquinazolin-1(2H)-yl)-2,3-difluorophenyl)butyl)carbamate 131g (71 mg, 31% yield) was prepared. HPLC/MS 1.45 min (A), [M+H]⁺ 703.2. ¹H NMR (CD₃OD, 400 MHz): δ 7.90 (br d, J=10.3 Hz, 1H), 7.68-7.79 (m, 1H), 7.17-7.39 (m, 2H), 6.85-6.93 (m, 1H), 6.45-6.59 (m, 1H), 5.42-5.56 (m, 1H), 5.03-5.15 (m, 1H), 3.95 (s, 3H), 3.02-3.19 (m, 2H), 2.90-2.98 (m, 1H), 1.87-2.04 (m, 2H), 1.28-1.45 (m, 12H).

### Step H: 1-(2-(3-Aminobutyl)-3,4-difluorophenyl)-3-(2-bromo-6-methoxypyridin-3-yl)-6-fluoro-7-(trifluoromethyl)-2,3-dihydroquinazolin-4(1H)-one 131h

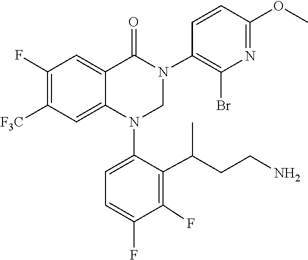 [SMILES: COc1ccc(N2CN(c3ccc(F)c(F)c3C(C)CCN)c3cc(C(F)(F)F)c(F)cc3C2=O)c(Br)n1]

Following the procedure outlined in Example 129, Step E, stirring the reaction at rt for 18 h, 1-(2-(3-aminobutyl)-3,4-difluorophenyl)-3-(2-bromo-6-methoxypyridin-3-yl)-6-fluoro-7-(trifluoromethyl)-2,3-dihydroquinazolin-4(1H)-one 131h (43 mg, 70% yield) was prepared as a yellow oil, which was used without further purification. HPLC/MS 0.93 min (A), [M+H]⁺ 604.9.

### Step I: 2,8,9-Trifluoro-15-methoxy-10-methyl-3-(trifluoromethyl)-10,11,12,13-tetra-hydro-19H-5,18-methanodibenzo[f,i]pyrido[2,3-b][1,4,8]triazacyclotridecine-19-one 131i

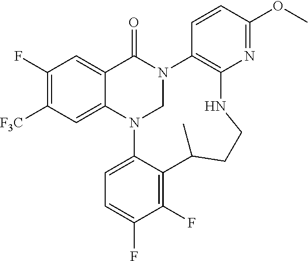 [SMILES: COc1ccc2c(n1)NCCC(C)c1c(ccc(F)c1F)N1CN2C(=O)c2cc(F)c(C(F)(F)F)cc21]

Following the procedure outlined in Example 129, Step F, stirring the reaction at 100° C. for 2 h, 2,8,9-trifluoro-15-methoxy-10-methyl-3-(trifluoromethyl)-10,11,12,13-tetrahydro-19H-5,18-methanodibenzo[f,i]pyrido[2,3-b][1,4,8]triazacyclotridecine-19-one 131i (26.0 mg, 77% yield) was prepared as a yellow solid. HPLC/MS 1.35 min (A), [M+H]⁺ 523.2. ¹H NMR (CDCl₃, 400 MHz): δ 1.43 (dd, J=6.85, 1.47 Hz, 3H), 2.00-2.09 (m, 2H), 3.30 (dt, J=13.33, 6.30 Hz, 2H), 3.61 (dt, J=12.96, 5.26 Hz, 1H), 3.79-4.00 (m, 3H), 4.28 (d, J=11.74 Hz, 1H), 5.29-5.50 (m, 1H), 6.15 (d, J=8.31 Hz, 1H), 6.69 (d, J=5.38 Hz, 1H), 6.89-7.07 (m, 1H), 7.22 (q, J=8.80 Hz, 1H), 7.46 (d, J=8.31 Hz, 1H), 8.01 (d, J=10.27 Hz, 1H).

### Step J: 2,8,9-Trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione Example 131

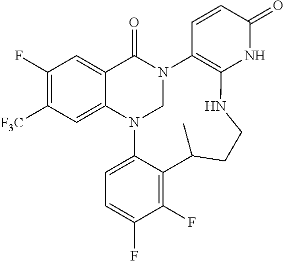 [SMILES: CC1CCNc2[nH]c(=O)ccc2N2CN(c3cc(C(F)(F)F)c(F)cc3C2=O)c2ccc(F)c(F)c21]

Following the procedure outlined in Example 27, Step F, stirring the reaction mixture at 90° C. for 44 h, 2,8,9-trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione (23.2 mg, 43% yield) was prepared as an off-white solid. HPLC/MS 1.11 min (A), [M+H]⁺ 509.2. ¹H NMR (CDCl₃, 400 MHz): δ 0.93 (br t, J=6.85 Hz, 3H), 1.17-1.49 (m, 6H), 1.88-2.21 (m, 2H), 3.12-3.38 (m, 3H), 3.62 (br s, 2H), 4.32 (br d, J=11.25 Hz, 2H), 4.94 (br s, 2H), 5.43 (br d, J=11.25 Hz, 2H), 6.02 (br d, J=8.80 Hz, 2H), 6.70 (br d, J=5.38 Hz, 2H), 6.91-7.15 (m, 2H), 7.18-7.29 (m, 2H), 7.33-7.44 (m, 2H), 7.97 (br d, J=9.78 Hz, 1H).

### Example 132 and Example 133

### Step A: (10S)-2,8,9-Trifluoro-10-methyl-3-(trifluoromethyl)-10,11,12,13-tetrahydro-19H-5,18-methanodibenzo[f,i]pyrido[2,3-b][1,4,8]triazacyclotridecin-15,19(14H)-dione (10R)-2,8,9-Trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione

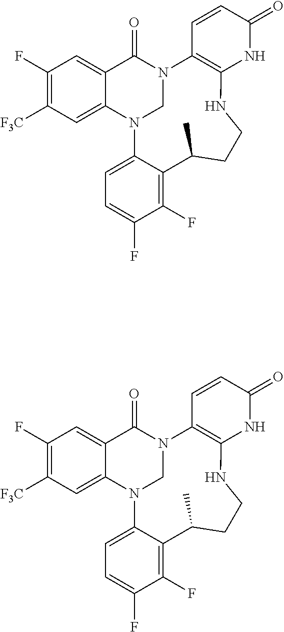 [SMILES: C[C@H]1CCNc2[nH]c(=O)ccc2N2CN(c3cc(C(F)(F)F)c(F)cc3C2=O)c2ccc(F)c(F)c21 ; C[C@@H]1CCNc2[nH]c(=O)ccc2N2CN(c3cc(C(F)(F)F)c(F)cc3C2=O)c2ccc(F)c(F)c21]

Racemic 2,8,9-trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione Example 131 (118 mg, 2.30 mmol) was resolved using chiral chromatography (Chiralpack AI column, 150×4.6 mm, 5 μm), eluting with an isocratic 1:1 heptanes-EtOH (0.1% formic acid) gradient to afford:

First-eluting stereoisomer (10S)-2,8,9-trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione Example 132 (40 mg, 34% yield). HPLC/MS 1.10 min (A), [M+H]⁺ 509.1. ¹H NMR (CD₂Cl₂, 400 MHz): δ 7.94 (d, J=10.3 Hz, 1H), 7.39 (d, J=8.8 Hz, 1H), 7.24 (q, J=8.8 Hz, 1H), 7.06 (ddd, J=8.8, 4.4, 2.0 Hz, 1H), 6.74 (d, J=5.4 Hz, 1H), 5.99 (d, J=9.3 Hz, 1H), 5.41 (d, J=11.7 Hz, 1H), 5.36 (s, 2H), 4.29 (d, J=11.7 Hz, 1H), 3.52-3.61 (m, 1H), 3.20-3.33 (m, 2H), 1.89-2.07 (m, 2H), 1.35 (d, J=6.4 Hz, 3H).

Second-eluting stereoisomer (10R)-2,8,9-trifluoro-10-methyl-3-(trifluoromethyl)-10,11,12,13-tetrahydro-19H-5,18-methanodibenzo[f,i]pyrido[2,3-b][1,4,8]triazacyclotridecine-15,19(14H)-dione Example 133 (44 mg, 37% yield). HPLC/MS 1.10 min (A), [M+H]⁺ 509.2. ¹H NMR (CD₂Cl₂, 400 MHz): δ 7.96 (d, J=9.8 Hz, 1H), 7.45 (br d, J=8.8 Hz, 1H), 7.19-7.31 (m, 1H), 7.05 (ddd, J=8.8, 4.4, 2.0 Hz, 1H), 6.74 (d, J=5.4 Hz, 1H), 6.03 (br d, J=9.3 Hz, 1H), 5.42 (d, J=11.7 Hz, 1H), 5.36 (s, 2H), 4.30 (d, J=11.2 Hz, 1H), 3.55-3.64 (m, 1H), 3.21-3.31 (m, 2H), 1.96-2.07 (m, 2H), 1.36 (dd, J=6.8, 1.5 Hz, 3H).

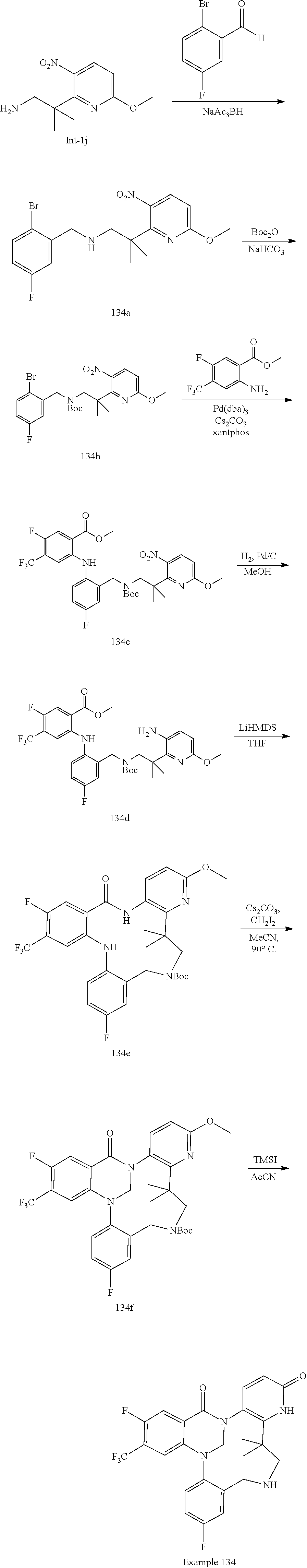

### Example 134

### 2,8-Difluoro-13,13-dimethyl-3-(trifluoromethyl)-10,11,12,13-tetrahydro-19H-5,18-methanodibenzo[c,f]pyrido[3,2-j][1,5,9]triazacyclotridecine-15,19(14H)-dione

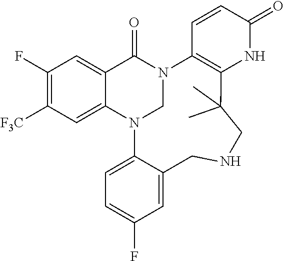 [SMILES: CC1(C)CNCc2cc(F)ccc2N2CN(C(=O)c3cc(F)c(C(F)(F)F)cc32)c2ccc(=O)[nH]c21]

### Step A: N-(2-Bromo-5-fluorobenzyl)-2-(6-methoxy-3-nitropyridin-2-yl)-2-methylpropan-1-amine 134a

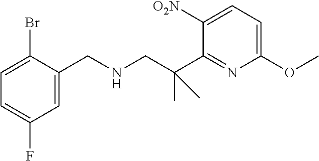 [SMILES: COc1ccc([N+](=O)[O-])c(C(C)(C)CNCc2cc(F)ccc2Br)n1]

To (2-bromo-5-fluorophenyl)methanamine (2.50 g, 12.3 mmol) and 2-(6-methoxy-3-nitropyridin-2-yl)-2-methylpropanal Int-1j (2.75 g, 12.3 mmol) in DCE (30 mL) was added AcOH (0.070 mL, 1.23 mmol), the reaction mixture stirred for 2 h, to which was added sodium triacetoxyborohydride (7.79 g, 36.8 mmol) and the reaction mixture stirred at RT for 16 h. The reaction mixture was quenched with aq. sat'd NaHCO₃ (40 mL), extracted with DCM (2×40 mL), the combined extracts dried over Na₂SO₄, filtered and concentrated under reduced pressure to afford crude N-(2-bromo-5-fluorobenzyl)-2-(6-methoxy-3-nitropyridin-2-yl)-2-methylpropan-1-amine 134a (5.30 g, 94% yield) as brown liquid, which was used without further purification. HPLC/MS 0.719 min (C), [M+H]⁺ 412.0.

### Step B: tert-Butyl (2-bromo-5-fluorobenzyl)(2-(6-methoxy-3-nitropyridin-2-yl)-2-methylpropyl)carbamate 134b

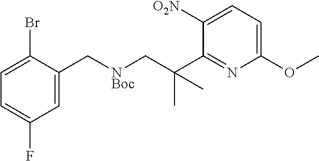 [SMILES: COc1ccc([N+](=O)[O-])c(C(C)(C)CN(Cc2cc(F)ccc2Br)C(=O)OC(C)(C)C)n1]

To N-(2-bromo-5-fluorobenzyl)-2-(6-methoxy-3-nitropyridin-2-yl)-2-methylpropan-1-amine 134a (3.20 g, 7.76 mmol) in DCM (30.0 mL) and H₂O (5.00 mL) was added sodium bicarbonate (0.978 g, 11.6 mmol). After 10 min, boc-anhydride (2.70 mL, 11.6 mmol) was added and the reaction mixture was stirred for 16 h. The reaction mixture was diluted with DCM (30 mL), washed with H₂O (20 mL), brine (20 mL), dried over Na₂SO₄, filtered, and concentrated under reduced pressure. The crude residue was dissolved in DCM (4 mL), adsorbed onto silica gel (1.5 g) and purified by silica gel flash column chromatography, eluting with a 5-10% ethyl acetate-pet ether gradient. Product fractions were combined and evaporated under reduced pressure to afford tert-butyl (2-bromo-5-fluorobenzyl)(2-(6-methoxy-3-nitropyridin-2-yl)-2-methylpropyl)carbamate 134b (1.53 g, 38% yield) as a light brown liquid. HPLC/MS 1.529 min (C), [M-Boc]⁺ 412.0.

### Step C: Methyl 2-((2-(((tert-butoxycarbonyl)(2-(6-methoxy-3-nitropyridin-2-yl)-2-methylpropyl)amino)methyl)-4-fluorophenyl)amino)-5-fluoro-4-(trifluoromethyl)benzoate 134c

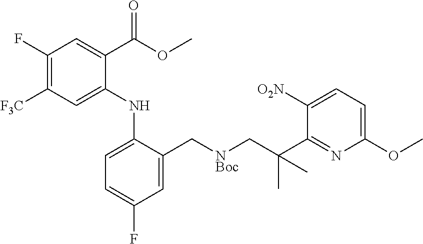 [SMILES: COC(=O)c1cc(F)c(C(F)(F)F)cc1Nc1ccc(F)cc1CN(CC(C)(C)c1nc(OC)ccc1[N+](=O)[O-])C(=O)OC(C)(C)C]

Following the procedure outlined in Example 44, Step A, substituting 2-bromo-5-fluorobenzaldehyde with tert-butyl (2-bromo-5-fluorobenzyl)(2-(6-methoxy-3-nitropyridin-2-yl)-2-methylpropyl)carbamate 134b, Pd(OAc)₂ with Pd₂(dba)₃, and heating to 110° C. for 16 h, methyl 2-((2-(((tert-butoxycarbonyl)(2-(6-methoxy-3-nitropyridin-2-yl)-2-methylpropyl)amino)methyl)-4-fluorophenyl)amino)-5-fluoro-4-(trifluoromethyl)benzoate 134c (1.30 g, 33% yield) was prepared as a colorless gummy liquid. HPLC/MS 1.62 min (C), [M-Boc]⁺ 569.3.

### Step D: Methyl 2-((2-(((2-(3-amino-6-methoxypyridin-2-yl)-2-methylpropyl)(tert-butoxycarbonyl)amino)methyl)-4-fluorophenyl)amino)-5-fluoro-4-(trifluoromethyl)benzoate 134d

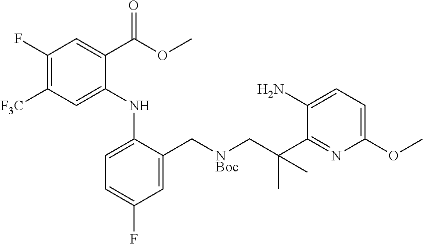 [SMILES: COC(=O)c1cc(F)c(C(F)(F)F)cc1Nc1ccc(F)cc1CN(CC(C)(C)c1nc(OC)ccc1N)C(=O)OC(C)(C)C]

Following the procedure outlined in Example 44, Step D, stirring the reaction mixture for 24h, crude methyl 2-((2-(((2-(3-amino-6-methoxypyridin-2-yl)-2-methylpropyl)(tert-butoxy-carbonyl)amino)methyl)-4-fluorophenyl)amino)-5-fluoro-4-(trifluoromethyl)benzoate 134d (900 mg, 58% yield) was prepared as a gummy yellow solid, which was used without further purification. HPLC/MS 1.53 min (C), [M+H]⁺ 639.5.

### Step E: tert-Butyl 2,8-difluoro-15-methoxy-13,13-dimethyl-19-oxo-3-(trifluoromethyl)-5,10,12,13,18,19-hexahydro-11H-dibenzo[c,f]pyrido[3,2-j][1,5,9]triazacyclotridecine-11-carboxylate 134e

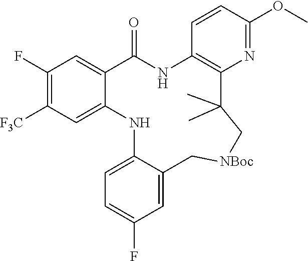 [SMILES: COc1ccc2c(n1)C(C)(C)CN(C(=O)OC(C)(C)C)Cc1cc(F)ccc1Nc1cc(C(F)(F)F)c(F)cc1C(=O)N2]

To methyl 2-((2-(((2-(3-amino-6-methoxypyridin-2-yl)-2-methylpropyl)(tert-butoxy-carbonyl)amino)methyl)-4-fluorophenyl)amino)-5-fluoro-4-(trifluoromethyl)benzoate 134d (900 mg, 1.41 mmol) in THF (10 mL) at −10° C. was added LiHMDS (3.52 mL, 3.52 mmol). The reaction mixture was warmed to ambient temperature, stirred for 16 h, quenched with aq. sat'd NH₄Cl (20 mL) and extracted with ethyl acetate (2×20 mL). The combined organic extracts were washed with brine (20 ml), dried over Na₂SO₄, filtered and concentrated under reduced pressure. The crude residue was dissolved in DCM (4 mL), adsorbed onto silica gel (1.5 g) and purified by silica gel flash column chromatography, eluting with a 20% ethyl acetate-pet ether to afford tert-butyl 2,8-difluoro-15-methoxy-13,13-dimethyl-19-oxo-3-(trifluoromethyl)-5,10,12,13,18,19-hexahydro-11H-dibenzo[c,f]pyrido[3,2-j][1,5,9]triazacyclotridecine-11-carboxylate 134e (430 mg, 46% yield) as a dark yellow solid. HPLC/MS 1.497 min (C), [M+H]⁺ 607.2

### Step F: tert-Butyl 2,8-difluoro-15-methoxy-13,13-dimethyl-19-oxo-3-(trifluoromethyl)-12,13-dihydro-19H-5,18-methanodibenzo[c,f]pyrido[3,2-j][1,5,9]triazacyclotridecine-11(10H)-carboxylate 134f

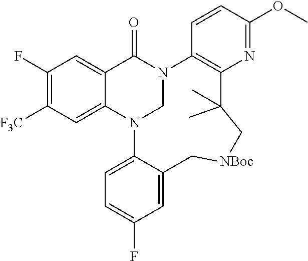 [SMILES: COc1ccc2c(n1)C(C)(C)CN(C(=O)OC(C)(C)C)Cc1cc(F)ccc1N1CN2C(=O)c2cc(F)c(C(F)(F)F)cc21]

Following the procedure outlined in Example 41, Step F, stirring the reaction at 80° C. for 16 h, tert-butyl 2,8-difluoro-15-methoxy-13,13-dimethyl-19-oxo-3-(trifluoromethyl)-12,13-dihydro-19H-5,18-methanodibenzo[c,f]pyrido[3,2-j][1,5,9]triazacyclotridecine-11(10H)-carboxylate 134f (183 mg, 35% yield) was prepared as a yellow solid. HPLC/MS 1.54 min (C), [M+H]⁺ 619.2.

### Step G: 2,8-Difluoro-13,13-dimethyl-3-(trifluoromethyl)-10,11,12,13-tetrahydro-19H-5,18-methanodibenzo[c,f]pyrido[3,2-j][1,5,9]triazacyclotridecine-15,19(14H)-dione

### Example 134

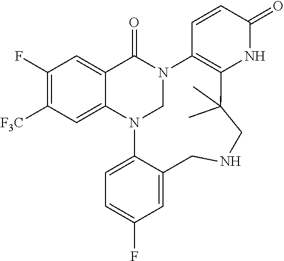 [SMILES: CC1(C)CNCc2cc(F)ccc2N2CN(C(=O)c3cc(F)c(C(F)(F)F)cc32)c2ccc(=O)[nH]c21]

To tert-butyl 2,8-difluoro-15-methoxy-13,13-dimethyl-19-oxo-3-(trifluoromethyl)-12,13-dihydro-19H-5,18-methanodibenzo[c,f]pyrido[3,2-j][1,5,9]triazacyclotridecine-11(10H)-carboxylate 134f (100 mg, 0.162 mmol) in MeCN (3 mL) was added iodotrimethylsilane (0.110 mL, 0.808 mmol) and the reaction mixture was stirred at 80° C. for 16 h. The reaction mixture was cooled to ambient temperature, quenched with sat'd Na₂S₂O₃ (10 mL) and extracted with ethyl acetate (2×10 mL). The combined organic extracts were dried over Na₂SO₄, filtered, and concentrated under reduced pressure. The crude residue was purified by reverse-phase semi-prep HPLC (Sunfire C18, 19×150 mm, 5 μm), eluting with AcCN in (0.1% HCOOH in water). Product fractions were combined and lyophilized to afford 2,8-difluoro-13,13-dimethyl-3-(trifluoromethyl)-10,11,12,13-tetrahydro-19H-5,18-methanodibenzo[c,f]pyrido[3,2-j][1,5,9]-triazacyclotridecine-15,19(14H)-dione (52 mg, 6300 yield) as an off-white solid. HPLC/MS 0.79 min (C), [M+H]⁺ 505.0. ¹H NMR (DMSO-d₆, 400 MHz): δ 0.76 (s, 1H), 7.84 (d, J=10.40 Hz, 1H), 7.56 (t, J=6.00 Hz, 1H), 7.43 (d, J=9.20 Hz, 1H), 7.29 (d, J=7.60 Hz, 2H), 6.48 (d, J=5.60 Hz, 1H), 6.33-6.31 (m, 1H), 5.5 5 (d, J=9.20 Hz, 1H), 4.81 (d, J=9.20 Hz, 1H), 3.66 (d, J=12.00 Hz, 1H), 3.34 (m, 1H), 2.92-2.89 (m, 1H), 2.69-2.67 (m, 1H), 2.34-2.33 (m, 1H), 1.35 (s, 3H), 1.23 (s, 3H).

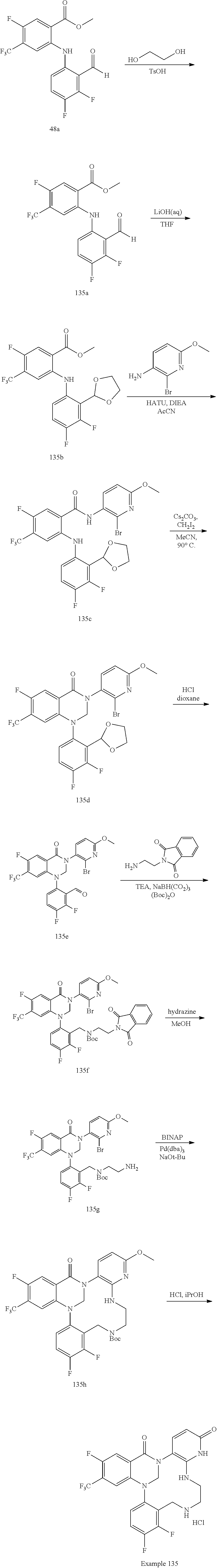

### Example 135

### 2⁶,3³,3⁴-Trifluoro-2⁷-(trifluoromethyl)-1′ 1²,2¹,2²,2³ 2⁴-hexahydro-5,8-diaza-2(3,1)-quinazolina-1(5,6)-pyridina-3(1,2)-benzenacyclooctaphane-1²,2⁴-dione hydrochloride

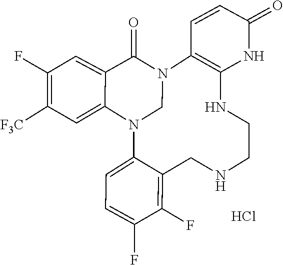 [SMILES: O=C1c2cc(F)c(C(F)(F)F)cc2N2CN1c1ccc(=O)[nH]c1NCCNCc1c2ccc(F)c1F ; Cl]

### Step A: Methyl 2-((2-(1,3-dioxolan-2-yl)-3,4-difluorophenyl)amino)-5-fluoro-4-(trifluoromethyl)benzoate 135a

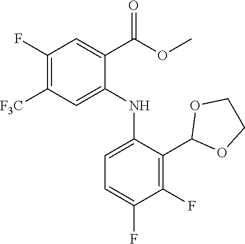 [SMILES: COC(=O)c1cc(F)c(C(F)(F)F)cc1Nc1ccc(F)c(F)c1C1OCCO1]

A mixture of methyl 2-((3,4-difluoro-2-formylphenyl)amino)-5-fluoro-4-(trifluoro-methyl)benzoate 48a (1.00 g, 2.65 mmol), ethylene glycol (443 μL, 7.95 mmol) andp-TSOH (25.2 mg, 133 μmol) in toluene (100 mL) was heated at 130° C. over the weekend, cooled to ambient temperature, and concentrated under reduced pressure. The residue was diluted with ether and H₂O, the layers separated, and the aqueous layer extracted with ether. The combined organic extracts were washed with brine, dried over MgSO₄, filtered, and concentrated under reduced pressure. The crude residue was dissolved in DCM, adsorbed onto silica gel and purified silica gel flash column chromatography (120 g), eluting with a 100% heptanes to 100% EtOAc gradient. Product fractions were combined and evaporated under reduced pressure. Hexanes was added to the residue and the product triturated and filtered to afford methyl 2-((2-(1,3-dioxolan-2-yl)-3,4-difluorophenyl)amino)-5-fluoro-4-(trifluoromethyl)benzoate 135a (859 mg, 70% yield) as a yellow solid. HPLC/MS 1.38 min (B), [M+H]⁺ 422.1. ¹H NMR (CDCl₃, 400 MHz): δ 9.65 (br s, 1H), 7.77 (d, J=10.8 Hz, 1H), 7.37-7.41 (m, 1H), 7.14-7.23 (m, 1H), 7.12 (br dd, J=4.2, 1.7 Hz, 1H), 6.16 (s, 1H), 4.18-4.26 (m, 2H), 4.01-4.08 (m, 2H), 3.96 (s, 3H).

### Step B: 2-((2-(1,3-Dioxolan-2-yl)-3,4-difluorophenyl)amino)-5-fluoro-4-(trifluoro-methyl)benzoic acid 135b

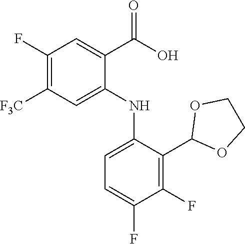 [SMILES: O=C(O)c1cc(F)c(C(F)(F)F)cc1Nc1ccc(F)c(F)c1C1OCCO1]

Following the procedure outlined in Example 27, Step B, 2-((2-(1,3-dioxolan-2-yl)-3,4-difluorophenyl)amino)-5-fluoro-4-(trifluoromethyl)benzoic acid 135b (712 mg, 83% yield) was prepared as a yellow solid. HPLC/MS 0.87 min (B), [M+H]⁺ 408.1. ¹H NMR (DMSO-d₆, 400 MHz): δ 9.76 (s, 1H) 7.85 (d, J=11.2 Hz, 1H) 7.53 (br d, J=9.8 Hz, 1H) 7.35 (br d, J=3.9 Hz, 1H) 7.24 (d, J=5.9 Hz, 1H) 6.02 (s, 1H) 3.99-4.10 (m, 2H) 3.89-3.99 (m, 2H).

### Step C: 2-((2-(1,3-Dioxolan-2-yl)-3,4-difluorophenyl)amino)-N-(2-bromo-6-methoxy-pyridin-3-yl)-5-fluoro-4-(trifluoromethyl)benzamide 135c

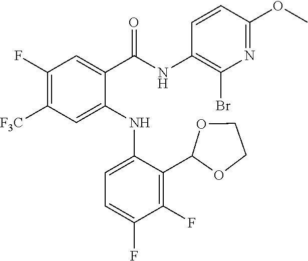 [SMILES: COc1ccc(NC(=O)c2cc(F)c(C(F)(F)F)cc2Nc2ccc(F)c(F)c2C2OCCO2)c(Br)n1]

Following the procedure outlined in Example 27, Step D, using 2-((2-(1,3-dioxolan-2-yl)-3,4-difluorophenyl)amino)-5-fluoro-4-(trifluoromethyl)benzoic acid 135b and 2-bromo-6-methoxypyridin-3-amine as substrates, substituting pyoxim with HATU and stirring the reaction mixture at room temperature overnight, 2-((2-(1,3-dioxolan-2-yl)-3,4-difluorophenyl)amino)-N-(2-bromo-6-methoxypyridin-3-yl)-5-fluoro-4-(trifluoromethyl)benzamide 135c (608 mg, 41% yield) was prepared as an orange solid. HPLC/MS 1.49 min (B), [M+H]⁺ 592.0, 594.0. ¹H NMR (CDCl₃, 400 MHz): δ 9.14 (s, 1H), 8.47 (d, J=8.3 Hz, 1H), 8.28 (s, 1H), 7.45-7.59 (m, 2H), 7.17 (br d, J=8.8 Hz, 1H), 6.97-7.08 (m, 1H), 6.80 (d, J=8.8 Hz, 1H), 6.16 (s, 1H), 4.27-4.38 (m, 2H), 4.02-4.09 (m, 2H), 3.96 (s, 3H).

### Step D: 1-(2-(1,3-Dioxolan-2-yl)-3,4-difluorophenyl)-3-(2-bromo-6-methoxypyridin-3-yl)-6-fluoro-7-(trifluoromethyl)-2,3-dihydroquinazolin-4(11H)-one 135d

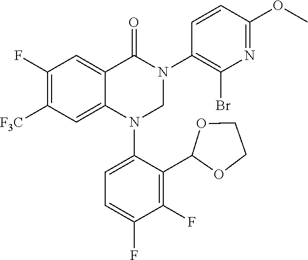 [SMILES: COc1ccc(N2CN(c3ccc(F)c(F)c3C3OCCO3)c3cc(C(F)(F)F)c(F)cc3C2=O)c(Br)n1]

Following the procedure outlined in Example 27, Step E, stirring the reaction mixture at 80° C. for 2 days, 1-(2-(1,3-dioxolan-2-yl)-3,4-difluorophenyl)-3-(2-bromo-6-methoxypyridin-3-yl)-6-fluoro-7-(trifluoromethyl)-2,3-dihydroquinazolin-4(1H)-one 135d (429 mg, 60% yield) was prepared as a light orange solid. HPLC/MS 1.40 min (B), [M+H]⁺ 604.0, 606.0. ¹H NMR (CDCl₃, 400 MHz): δ 7.94 (d, J=10.3 Hz, 1H), 7.30-7.39 (m, 1H), 7.23-7.30 (m, 1H), 7.10 (br s, 1H), 6.85 (br s, 1H), 6.69-6.82 (m, 1H), 6.09-6.32 (m, 1H), 4.97-5.28 (m, 2H), 4.21 (br s, 1H), 3.92-4.10 (m, 6H).

### Step E: 6-(3-(2-Bromo-6-methoxypyridin-3-yl)-6-fluoro-4-oxo-7-(trifluoromethyl)-3,4-dihydroquinazolin-1(2H)-yl)-2,3-difluorobenzaldehyde 135e

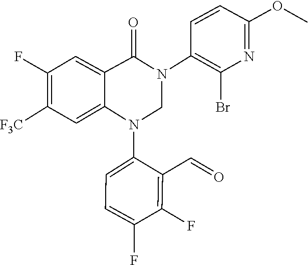 [SMILES: COc1ccc(N2CN(c3ccc(F)c(F)c3C=O)c3cc(C(F)(F)F)c(F)cc3C2=O)c(Br)n1]

To 1-(2-(1,3-dioxolan-2-yl)-3,4-difluorophenyl)-3-(2-bromo-6-methoxypyridin-3-yl)-6-fluoro-7-(trifluoromethyl)-2,3-dihydroquinazolin-4(1H)-one 135d (379 mg, 627 μmol) in dioxane (5 mL) and water (5 mL) was added 4N HCl in dioxane (4.70 mL, 18.8 mmol). The reaction mixture was stirred for 1 h at 50° C. and diluted with EtOAc and H₂O. The layers were separated and the aqueous layer was extracted with EtOAc. The combined organic extracts were washed with brine, dried over MgSO₄, filtered and the solvent evaporated under reduced pressure. The crude residue was adsorbed on a silica gel and purified by silica gel flash column chromatography (40 g), eluting with a 100% heptanes to 100% EtOAc gradient. Pure product fractions were combined and evaporated under reduced pressure to afford 6-(3-(2-bromo-6-methoxypyridin-3-yl)-6-fluoro-4-oxo-7-(trifluoromethyl)-3,4-dihydroquinazolin-1(2H)-yl)-2,3-difluoro-benzaldehyde 135e (240 mg, 84% yield) as a yellow solid. HPLC/MS 1.33 min (B), [M+H]⁺ 560.0, 562.0. ¹H NMR (CD₂Cl₂, 400 MHz): δ 10.44 (s, 1H), 7.99 (d, J=9.8 Hz, 1H), 7.47 (d, J=8.8 Hz, 2H), 7.02 (d, J=5.4 Hz, 2H), 6.71-6.86 (m, 1H), 5.30-5.60 (m, 1H), 4.55-4.87 (m, 1H), 3.94 (s, 3H).

### Step F: tert-Butyl (6-(3-(2-bromo-6-methoxypyridin-3-yl)-6-fluoro-4-oxo-7-(trifluoro-methyl)-3,4-dihydroquinazolin-1(2H)-yl)-2,3-difluorobenzyl)(2-(1,3-dioxoisoindolin-2-yl)ethyl)-carbamate 135f

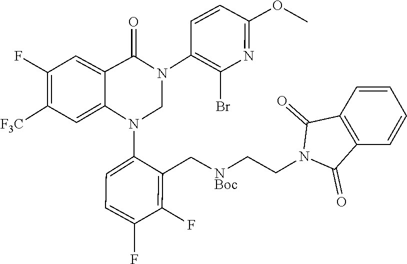 [SMILES: COc1ccc(N2CN(c3ccc(F)c(F)c3CN(CCN3C(=O)c4ccccc4C3=O)C(=O)OC(C)(C)C)c3cc(C(F)(F)F)c(F)cc3C2=O)c(Br)n1]

Following the preparation outlined in Example 44, Step B, using 6-(3-(2-bromo-6-methoxypyridin-3-yl)-6-fluoro-4-oxo-7-(trifluoromethyl)-3,4-dihydroquinazolin-1(2H)-yl)-2,3-difluorobenzaldehyde 135e as substrates, tert-butyl (6-(3-(2-bromo-6-methoxypyridin-3-yl)-6-fluoro-4-oxo-7-(trifluoromethyl)-3,4-dihydroquinazolin-1(2H)-yl)-2,3-difluorobenzyl)(2-(1,3-dioxoisoindolin-2-yl)ethyl)carbamate 135f (391 mg, 71% yield) was prepared as a white solid. HPLC/MS 1.55 min (B), [M+H]⁺ 834.0, 836.0. ¹H NMR (CDCl₃, 400 MHz): δ 7.67-7.92 (m, 6H), 7.14-7.26 (m, 1H), 7.11 (br s, 1H), 6.72 (br d, J=7.8 Hz, 1H), 6.42 (br s, 1H), 5.18-5.39 (m, 1H), 5.11 (br d, J=9.3 Hz, 1H), 4.73 (br d, J=14.2 Hz, 1H), 4.60 (br d, J=14.7 Hz, 1H), 3.77-3.95 (m, 5H), 3.45-3.71 (m, 1H), 3.32 (br s, 1H), 0.99-1.18 (m, 9H).

### Step G: tert-Butyl (2-aminoethyl)(6-(3-(2-bromo-6-methoxypyridin-3-yl)-6-fluoro-4-oxo-7-(trifluoromethyl)-3,4-dihydroquinazolin-1(2H)-yl)-2,3-difluorobenzyl)carbamate 1352

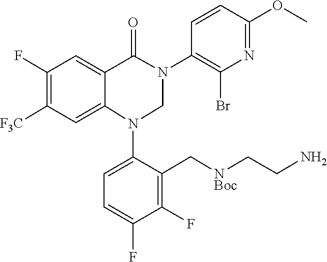 [SMILES: COc1ccc(N2CN(c3ccc(F)c(F)c3CN(CCN)C(=O)OC(C)(C)C)c3cc(C(F)(F)F)c(F)cc3C2=O)c(Br)n1]

Following the preparation outlined in Example 104, Step E, stirring at RT overnight, tert-butyl (2-aminoethyl)(6-(3-(2-bromo-6-methoxypyridin-3-yl)-6-fluoro-4-oxo-7-(trifluoro-methyl)-3,4-dihydroquinazolin-1(2H)-yl)-2,3-difluorobenzyl)carbamate 135g (192 mg, 77% yield) was prepared as a colorless oil. HPLC/MS 1.35 min (B), [M+H]⁺ 704.1, 706.1. ¹H NMR (CDCl₃, 400 MHz): δ 7.79 (br d, J=9.8 Hz, 1H), 7.68 (br s, 1H), 7.05-7.15 (m, 1H), 6.99 (br s, 1H), 6.65 (br s, 1H), 6.28-6.46 (m, 1H), 5.16 (br s, 1H), 4.83-5.06 (m, 1H), 4.65-4.82 (m, 1H), 4.35 (br s, 1H), 3.82 (s, 3H), 3.20 (br s, 2H), 2.62-2.92 (m, 2H), 1.08-1.22 (m, 9H).

### Step H: tert-Butyl 2⁶,3³,3⁴-trifluoro-1⁶-methoxy-2⁴-oxo-2⁷-(trifluoromethyl)-2¹,2²,2³ 2⁴-tetrahydro-5,8-diaza-2(3,1)-quinazolina-1(3,2)-pyridina-3(1,2)-benzenacyclooctaphane-5-carboxylate 135h

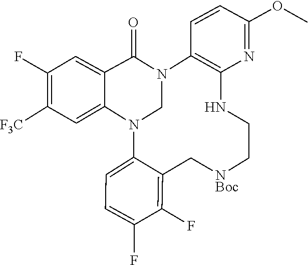 [SMILES: COc1ccc2c(n1)NCCN(C(=O)OC(C)(C)C)Cc1c(ccc(F)c1F)N1CN2C(=O)c2cc(F)c(C(F)(F)F)cc21]

Following the procedure outlined in Example 129, Step F, stirring the reaction at 100° C. for 1.5 h, tert-butyl 2⁶,3³,3⁴-trifluoro-1⁶-methoxy-2⁴-oxo-2⁷-(trifluoromethyl)-2¹,2²,2³,2⁴-tetrahydro-5,8-diaza-2(3,1)-quinazolina-1(3,2)-pyridina-3(1,2)-benzenacyclooctaphane-5-carboxylate 135h (15.0 mg, 8% yield) was prepared. HPLC/MS 1.48 min (B), [M+H]⁺ 624.2. ¹H NMR (CDCl₃, 400 MHz): δ 7.80 (d, J=9.8 Hz, 1H), 7.35 (d, J=8.3 Hz, 1H), 7.16-7.30 (m, 2H), 6.87 (ddd, J=8.8, 4.2, 1.7 Hz, 1H), 6.22 (d, J=5.4 Hz, 1H), 5.98 (d, J=8.3 Hz, 1H), 5.24 (d, J=10.3 Hz, 1H), 5.13 (br d, J=14.7 Hz, 1H), 5.01 (br d, J=8.8 Hz, 1H), 4.43 (d, J=10.3 Hz, 1H), 4.19 (br d, J=14.7 Hz, 1H), 3.88 (br d, J=15.7 Hz, 1H), 3.72 (s, 3H), 3.54-3.67 (m, 1H), 3.26 (br d, J=11.7 Hz, 1H), 2.34-2.48 (m, 1H), 1.31-1.39 (m, 9H).

### Step I: 2⁶,3³,3⁴-Trifluoro-2⁷-(trifluoromethyl)-1¹,1²,2¹,2²,2³,2⁴-hexahydro-5,8-diaza-2(3,1)-quinazolina-1(5,6)-pyridina-3(1,2)-benzenacyclooctaphane-1²,2⁴-dione hydrochloride

### Example 135

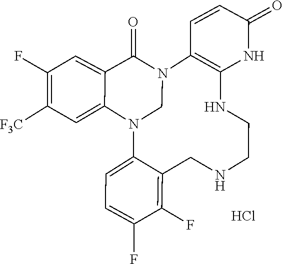 [SMILES: O=C1c2cc(F)c(C(F)(F)F)cc2N2CN1c1ccc(=O)[nH]c1NCCNCc1c2ccc(F)c1F ; Cl]

Following the procedure outlined in Example 27, Step F, stirring the reaction mixture at 90° C. overnight, 2⁶,3³,3⁴-trifluoro-2⁷-(trifluoromethyl)-1¹,1²,2¹,2²,2³,2⁴-hexahydro-5,8-diaza-2(3,1)-quinazolina-1(5,6)-pyridina-3(1,2)-benzenacyclooctaphane-1²,2⁴-dione hydrochloride (5.3 mg, 39% yield) was prepared as a white solid. HPLC/MS 1.01 min (A), [M+H]⁺ 510.2. ¹H NMR (CDCl₃, 400 MHz): δ 7.90 (br dd, J=9.8, 2.4 Hz, 1H), 7.36 (br s, 1H), 7.04 (br dd, J=8.1, 3.2 Hz, 1H), 6.45 (br d, J=5.4 Hz, 1H), 5.83 (br d, J=8.3 Hz, 1H), 5.50 (br s, 1H), 5.21 (br d, J=9.3 Hz, 1H), 4.65-4.78 (m, 1H), 4.26 (br d, J=12.2 Hz, 1H), 3.65 (br s, 1H), 3.38-3.50 (m, 1H), 3.15-3.38 (m, 2H), 2.78 (br s, 1H).

### Example 136 and Example 137

### (4R)-2⁶,3⁴-Difluoro-4-methyl-2⁷-(trifluoromethyl)-1¹,1²,2¹,2²,2³ 2⁴-hexahydro-5-aza-2(3,1)-quinazolina-1(5,6)-pyridina-3(1,2)-benzenacyclooctaphane-1²,2⁴-dione

### (4S)-2⁶,3⁴-Difluoro-4-methyl-2⁷-(trifluoromethyl)-1¹,1²,2¹,2²,2³ 2⁴-hexahydro-5-aza-2(3,1)-quinazolina-1(5,6)-pyridina-3(1,2)-benzenacyclooctaphane-1²,2⁴-dione

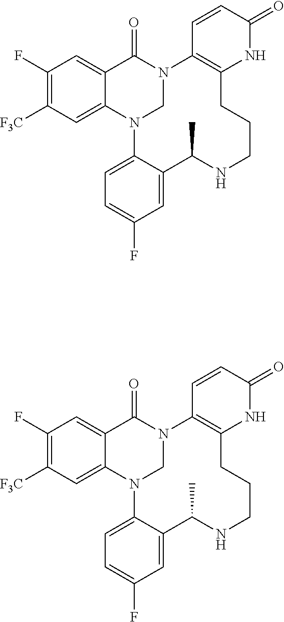 [SMILES: C[C@H]1NCCCc2[nH]c(=O)ccc2N2CN(c3cc(C(F)(F)F)c(F)cc3C2=O)c2ccc(F)cc21 ; C[C@@H]1NCCCc2[nH]c(=O)ccc2N2CN(c3cc(C(F)(F)F)c(F)cc3C2=O)c2ccc(F)cc21]

### Step A: Ethyl 2-((2-(1-((tert-butoxycarbonyl)(3-(6-methoxy-3-nitropyridin-2-yl)propyl)-amino)ethyl)-4-fluorophenyl)amino)-5-fluoro-4-(trifluoromethyl)benzoate 136a/137a

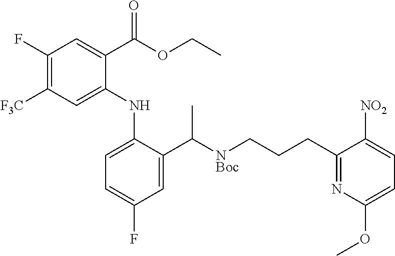 [SMILES: CCOC(=O)c1cc(F)c(C(F)(F)F)cc1Nc1ccc(F)cc1C(C)N(CCCc1nc(OC)ccc1[N+](=O)[O-])C(=O)OC(C)(C)C]

Following the preparation outlined in Example 120, Step B, using methyl 2-((2-acetyl-4-fluorophenyl)amino)-5-fluoro-4-(trifluoromethyl)benzoate 117a and 3-(6-methoxy-3-nitro-pyridin-2-yl)propan-1-amine hydrochloride Int-1h as substrates, and stirring at 70° C. for 18.5 h, ethyl 2-((2-(1-((tert-butoxycarbonyl)(3-(6-methoxy-3-nitropyridin-2-yl)propyl)amino)ethyl)-4-fluorophenyl)amino)-5-fluoro-4-(trifluoromethyl)benzoate 136a/137a (2.18 g, 63% yield) was prepared as a light yellow foam. HPLC/MS 1.72 min (A), [M+H]⁺ 683.3. ¹H NMR (CDCl₃, 400 MHz): δ 9.09 (br s, 1H), 8.23 (d, 1H, J=9.3 Hz), 7.7-7.9 (m, 1H), 7.2-7.3 (m, 1H), 7.15 (dd, 1H, J=2.7, 9.5 Hz), 7.04 (dt, 1H, J=2.9, 8.1 Hz), 6.92 (br d, 1H, J=5.9 Hz), 6.68 (d, 1H, J=9.3 Hz), 5.4-5.8 (m, 1H), 4.3-4.5 (m, 2H), 3.98 (s, 3H), 3.1-3.3 (m, 1H), 2.8-3.1 (m, 3H), 1.8-2.1 (m, 1H), 1.49 (d, 3H, J=7.3 Hz), 1.43 (t, 3H, J=7.1 Hz), 1.39 (s, 9H).

### Step B: 2-((2-(1-((tert-Butoxycarbonyl)(3-(6-methoxy-3-nitropyridin-2-yl)propyl)-amino)ethyl)-4-fluorophenyl)amino)-5-fluoro-4-(trifluoromethyl)benzoic acid 136b/137b

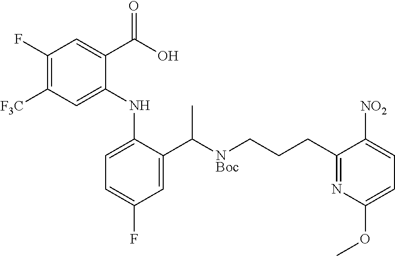 [SMILES: COc1ccc([N+](=O)[O-])c(CCCN(C(=O)OC(C)(C)C)C(C)c2cc(F)ccc2Nc2cc(C(F)(F)F)c(F)cc2C(=O)O)n1]

Following the procedure outlined in Example 27, Step B, stirring the reaction mixture at 50° C. for 28 h, 2-((2-(1-((tert-butoxycarbonyl)(3-(6-methoxy-3-nitropyridin-2-yl)propyl)amino)-ethyl)-4-fluorophenyl)amino)-5-fluoro-4-(trifluoromethyl)benzoic acid 136b/137b (2.04 g, 95% yield) was prepared as a yellow foam. HPLC/MS 1.59 min (A), [M+H]⁺ 655.2. ¹H NMR (DMSO-d₆, 400 MHz): δ 13.3-14.0 (m, 1H), 8.9-9.5 (m, 1H), 8.30 (d, 1H, J=9.3 Hz), 7.7-7.9 (m, 1H), 7.35 (s, 2H), 7.1-7.3 (m, 1H), 6.85 (d, 1H, J=8.8 Hz), 6.5-6.7 (m, 1H), 5.3-5.6 (m, 1H), 3.89 (s, 3H), 2.85 (d, 5H, J=7.3 Hz), 1.6-1.8 (m, 1H), 1.40 (br d, 3H, J=6.8 Hz), 1.1-1.3 (m, 12H).

### Step C: 2-((2-(1-((3-(3-Amino-6-methoxypyridin-2-yl)propyl)(tert-butoxycarbonyl)-amino)ethyl)-4-fluorophenyl)amino)-5-fluoro-4-(trifluoromethyl)benzoic acid 136c/137c

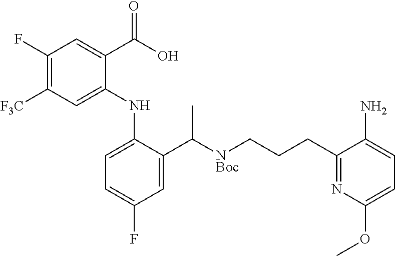 [SMILES: COc1ccc(N)c(CCCN(C(=O)OC(C)(C)C)C(C)c2cc(F)ccc2Nc2cc(C(F)(F)F)c(F)cc2C(=O)O)n1]

Following the procedure outlined in Example 44, Step D, partially pure 2-((2-(1-((3-(3-amino-6-methoxypyridin-2-yl)propyl)(tert-butoxycarbonyl)amino)ethyl)-4-fluorophenyl)amino)-5-fluoro-4-(trifluoromethyl)benzoic acid 136c/137c (1.66 g, 66% yield) was prepared as a light brown foam, which was used without further purification. HPLC/MS 1.12 min (A), [M+H]⁺ 625.2.

### Step D: tert-Butyl 2,8-difluoro-16-methoxy-10-methyl-20-oxo-3-(trifluoromethyl)-5,12,13,14,19,20-hexahydrodibenzo[c,f]pyrido[3,2-j][1,5,9]triazacyclotetradecine-11(10H)-carboxylate 136d/137d

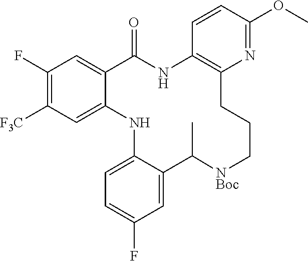 [SMILES: COc1ccc2c(n1)CCCN(C(=O)OC(C)(C)C)C(C)c1cc(F)ccc1Nc1cc(C(F)(F)F)c(F)cc1C(=O)N2]

Following the procedure outlined in Example 27, Step D, replacing pyoxim with HATU and stirring the reaction mixture at room temperature for 72 h, tert-butyl 2,8-difluoro-16-methoxy-10-methyl-20-oxo-3-(trifluoromethyl)-5,12,13,14,19,20-hexahydrodibenzo[c,f]pyrido[3,2-j][1,5,9]triazacyclotetradecine-11(10H)-carboxylate 136d/137d (0.757 g, 79% yield) was prepared as a white foam. HPLC/MS 1.53 min (A), [M+H]⁺ 607.2. ¹H NMR (DMSO-d₆, 400 MHz): δ 10.07 (s, 1H), 7.9-7.9 (m, 1H), 7.8-7.8 (m, 1H), 7.6-7.7 (m, 1H), 7.2-7.3 (m, 1H), 6.9-7.0 (m, 1H), 6.66 (d, 1H, J=8.8 Hz), 6.5-6.6 (m, 1H), 6.4-6.5 (m, 1H), 5.4-5.7 (m, 1H), 3.78 (s, 3H), 3.4-3.5 (m, 1H), 2.8-3.0 (m, 1H), 2.2-2.4 (m, 1H), 2.1-2.2 (m, 1H), 1.3-1.6 (m, 13H), 1.1-1.3 (m, 1H).

### Step E: tert-Butyl (4R)-2⁶,3⁴-difluoro-1⁶-methoxy-4-methyl-2⁴-oxo-2⁷-(trifluoromethyl)-2′,2²,2³,2⁴-tetrahydro-5-aza-2(3,1)-quinazolina-1(3,2)-pyridina-3(1,2)-benzenacyclooctaphane-5-carboxylate 136e tert-Butyl (4S)-2⁶,3⁴-difluoro-1⁶-methoxy-4-methyl-2⁴-oxo-2⁷-(trifluoromethyl)-2′,2²,2³,2⁴-tetrahydro-5-aza-2(3,1)-quinazolina-1(3,2)-pyridina-3(1,2)-benzenacyclooctaphane-5-carboxylate 137e

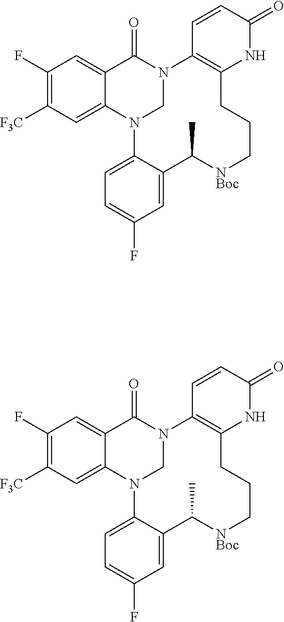 [SMILES: C[C@@H]1c2cc(F)ccc2N2CN(C(=O)c3cc(F)c(C(F)(F)F)cc32)c2ccc(=O)[nH]c2CCCN1C(=O)OC(C)(C)C ; C[C@H]1c2cc(F)ccc2N2CN(C(=O)c3cc(F)c(C(F)(F)F)cc32)c2ccc(=O)[nH]c2CCCN1C(=O)OC(C)(C)C]

Following the procedure outlined in Example 27, Step E, stirring the reaction mixture at 90° C. for 24 h, racemic tert-butyl 2⁶,3⁴-difluoro-1⁶-methoxy-4-methyl-2⁴-oxo-2⁷-(trifluoromethyl)-2¹,2²,2³,2⁴-tetrahydro-5-aza-2(3,1)-quinazolina-1(3,2)-pyridina-3(1,2)-benzenacyclooctaphane-5-carboxylate (629 mg, 80% yield) was prepared as a light yellow solid. HPLC/MS 1.45 min (A), [M+H]⁺ 619.2. ¹H NMR (CDCl₃, 400 MHz): δ 7.95 (d, 1H, J=9.8 Hz), 7.7-7.9 (m, 1H), 7.48 (d, 1H, J=8.3 Hz), 7.1-7.2 (m, 1H), 7.07 (br d, 1H, J=5.4 Hz), 6.67 (d, 1H, J=8.8 Hz), 6.48 (d, 1H, J=5.4 Hz), 5.66 (d, 1H, J=10.3 Hz), 4.7-4.8 (m, 1H), 4.63 (d, 1H, J=10.3 Hz), 3.94 (s, 3H), 3.36 (br d, 1H, J=7.3 Hz), 3.1-3.2 (m, 1H), 2.8-2.9 (m, 1H), 2.7-2.8 (m, 1H), 2.0-2.2 (m, 1H), 1.55 (d, 4H, J=6.8 Hz), 1.41 (s, 9H). This material was resolved using chiral chromatography (Chiralpack IB-N column, 150×4.6 mm, 5 μm), eluting with an isocratic 85:15 CO₂-MeOH gradient to afford:

First-eluting stereoisomer tert-butyl (4R)-2⁶,3⁴-difluoro-1⁶-methoxy-4-methyl-2⁴-oxo-2⁷-(trifluoromethyl)-2¹,2²,2³,2⁴-tetrahydro-5-aza-2(3,1)-quinazolina-1(3,2)-pyridina-3(1,2)-benzenacyclooctaphane-5-carboxylate 136e (299 mg, 38% yield); and

Second-eluting stereoisomer tert-butyl (4S)-2⁶,3⁴-difluoro-1⁶-methoxy-4-methyl-2⁴-oxo-2⁷-(trifluoromethyl)-2¹,2²,2³,2⁴-tetrahydro-5-aza-2(3,1)-quinazolina-1(3,2)-pyridina-3(1,2)-benzenacyclooctaphane-5-carboxylate 137e (299 mg, 38% yield).

### Step G: (4R)-2⁶,3⁴-Difluoro-4-methyl-2⁷-(trifluoromethyl)-1¹,1²,2¹,2²,2³ 2⁴-hexahydro-5-aza-2(3,1)-quinazolina-1(5,6)-pyridina-3(1,2)-benzenacyclooctaphane-1²,2⁴-dione

### (4S)-2⁶,3⁴-Difluoro-4-methyl-2⁷-(trifluoromethyl)-1¹,1²,2¹,2²,2³ 2⁴-hexahydro-5-aza-2(3,1)-quinazolina-1(5,6)-pyridina-3(1,2)-benzenacyclooctaphane-1²,2⁴-dione

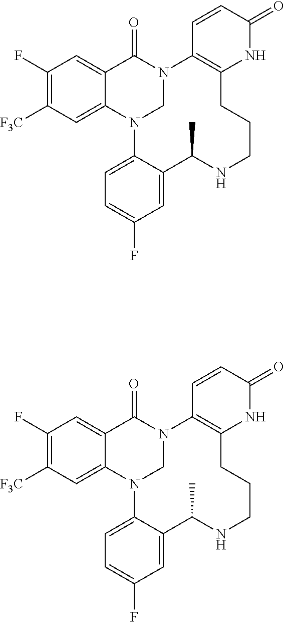 [SMILES: C[C@H]1NCCCc2[nH]c(=O)ccc2N2CN(c3cc(C(F)(F)F)c(F)cc3C2=O)c2ccc(F)cc21 ; C[C@@H]1NCCCc2[nH]c(=O)ccc2N2CN(c3cc(C(F)(F)F)c(F)cc3C2=O)c2ccc(F)cc21]

Following the procedure outlined in Example 27, Step F, stirring the reaction mixtures at 90° C. for 72 h, the following stereoisomers were prepared:

(4R)-2⁶,3⁴-Difluoro-4-methyl-2⁷-(trifluoromethyl)-1¹,1²,2¹,2²,2³,2⁴-hexahydro-5-aza-2(3,1)-quinazolina-1(5,6)-pyridina-3(1,2)-benzenacyclooctaphane-1²,2⁴-dione Example 136 (192 mg, 77% yield). HPLC/MS 0.70 min (A), [M+H]⁺ 505.2. ¹H NMR (CD₃OD, 400 MHz): δ 7.98 (d, 1H, J=10.3 Hz), 7.85 (d, 1H, J=9.8 Hz), 7.5-7.7 (m, 2H), 7.5-7.5 (m, 1H), 6.71 (d, 1H, J=9.3 Hz), 6.53 (d, 1H, J=5.4 Hz), 5.74 (d, 1H, J=11.2 Hz), 4.99 (d, 1H, J=11.2 Hz), 4.7-4.8 (m, 1H), 3.0-3.1 (m, 1H), 2.8-2.9 (m, 1H), 2.6-2.7 (m, 1H), 2.1-2.3 (m, 1H), 1.8-1.9 (m, 1H), 1.48 (d, 3H, J=6.8 Hz).

(4S)-2⁶,3⁴-Difluoro-4-methyl-2⁷-(trifluoromethyl)-1¹,1²,2¹,2²,2³,2⁴-hexahydro-5-aza-2(3,1)-quinazolina-1(5,6)-pyridina-3(1,2)-benzenacyclooctaphane-1²,2⁴-dione Example 137 (239 mg, 95% yield). HPLC/MS 0.70 min (A), [M+H]⁺ 505.2. ¹H NMR (CD₃OD, 400 MHz): δ 7.98 (d, 1H, J=10.3 Hz), 7.85 (d, 1H, J=9.8 Hz), 7.62 (br d, 1H, J=4.9 Hz), 7.58 (dd, 1H, J=2.9, 8.8 Hz), 7.5-7.5 (m, 1H), 6.72 (d, 1H, J=9.3 Hz), 6.5-6.6 (m, 1H), 5.74 (d, 1H, J=10.8 Hz), 4.99 (d, 1H, J=11.2 Hz), 4.7-4.8 (m, 1H), 3.2-3.3 (m, 1H), 3.0-3.1 (m, 1H), 2.8-2.9 (m, 1H), 2.6-2.7 (m, 1H), 2.2-2.3 (m, 1H), 1.8-1.9 (m, 1H), 1.48 (d, 3H, J=6.8 Hz).

The compounds in Table 1 below may be prepared using analogous procedures to those described in the Examples above.

TABLE 1

| Structure | Name |
|---|---|
| 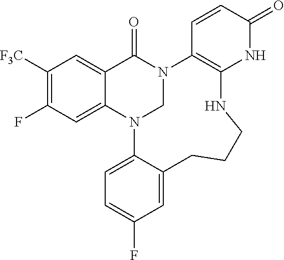 [SMILES: O=C1c2cc(C(F)(F)F)c(F)cc2N2CN1c1ccc(=O)[nH]c1NCCCc1cc(F)ccc12] | 3,8-Difluoro-2-(trifluoromethyl)-10,11,12,13-tetrahydro-19H- 5,18-methanodibenzo[f,i]pyrido[2,3- b][1,4,8]triazocyclotridecine-15,19(14H)-dione |
| 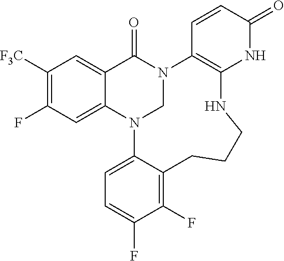 [SMILES: O=C1c2cc(C(F)(F)F)c(F)cc2N2CN1c1ccc(=O)[nH]c1NCCCc1c2ccc(F)c1F] | 3,8,9-Tiifluoro-2-(trifluoromethyl)-10,11,12,13-tetrahydro-19H- 5,18-methanodibenzo[f,i]pyrido[2,3- b][1,4,8]triazocyclotridecine-15,19(14H)-dione |
| 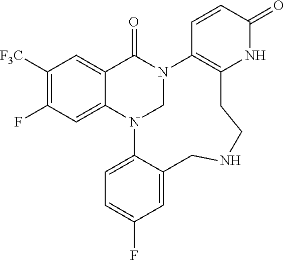 [SMILES: O=C1c2cc(C(F)(F)F)c(F)cc2N2CN1c1ccc(=O)[nH]c1CCNCc1cc(F)ccc12] | 3,8-Difluoro-2-(trifluoromethyl)-10,11,12,13-tetrahydro-19H- 5,18-methanodibenzo[c,f]pyrido[2,3- j][1,5,9]triazocyclotridecine-15,19(14H)-dione |
| 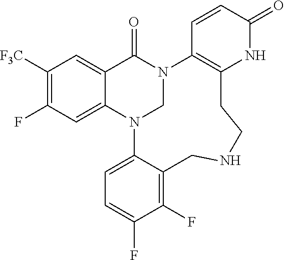 [SMILES: O=C1c2cc(C(F)(F)F)c(F)cc2N2CN1c1ccc(=O)[nH]c1CCNCc1c2ccc(F)c1F] | 3,8,9-Trifluoro-2-(trifluoromethyl)-10,11,12,13-tetrahydro-19H- 5,18-methanodibenzo[c,f]pyrido[2,3- j][1,5,9]triazocyclotridecine-15,19(14H)-dione |
| 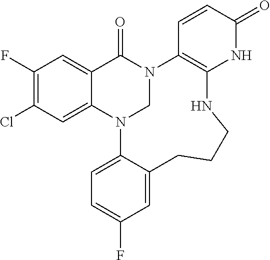 [SMILES: O=C1c2cc(F)c(Cl)cc2N2CN1c1ccc(=O)[nH]c1NCCCc1cc(F)ccc12] | 3-Chloro-2,8-difluoro-10,11,12,13-tetrahydro-19H-5,18- methanodibenzo[f,i]pyrido[2,3-b][1,4,8]triazocyclotridecine- 15,19(14H)-dione |
| 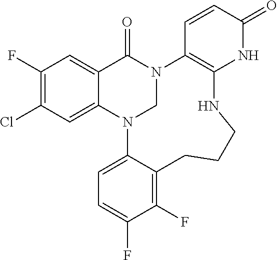 [SMILES: O=C1c2cc(F)c(Cl)cc2N2CN1c1ccc(=O)[nH]c1NCCCc1c2ccc(F)c1F] | 3-Chloro-2,8-diifluoro-10,11,12,13-tetrahydro-19H-5,18- methanodibenzo[f,i]pyrido[2,3-b][1,4,8]triazocyclotridecine- 15,19(14H)-dione |
| 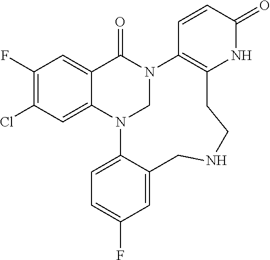 [SMILES: O=C1c2cc(F)c(Cl)cc2N2CN1c1ccc(=O)[nH]c1CCNCc1cc(F)ccc12] | 3-Chloro-2,8-difluoro-10,11,12,13-tetrahydro-19H-5,18- methanodibenzo[c,f]pyrido[2,3-j][1,5,9]triazocyclotridecine- 15,19(14H)-dione |
| 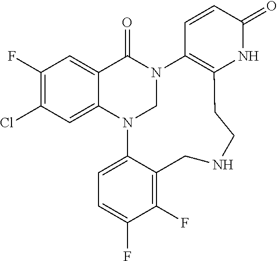 [SMILES: O=C1c2cc(F)c(Cl)cc2N2CN1c1ccc(=O)[nH]c1CCNCc1c2ccc(F)c1F] | 3-Chloro-2,8,9-trifluoro-10,11,12,13-tetrahydro-19H-5,18- methanodibenzo[c,f]pyrido[2,3-j][1,5,9]triazocyclotridecine- 15,19(14H)-dione |
| 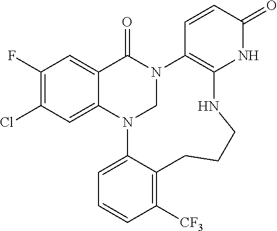 [SMILES: O=C1c2cc(F)c(Cl)cc2N2CN1c1ccc(=O)[nH]c1NCCCc1c2cccc1C(F)(F)F] | 3-Chloro-2-fluoro-9-(trifluoormethyl)-10,11,12,13-tetrahydro- 19H-5,18-methanodibenzo[f,i]pyrido[2,3- b][1,4,8]triazocyclotridecine-15,19(14H)-dione |
| 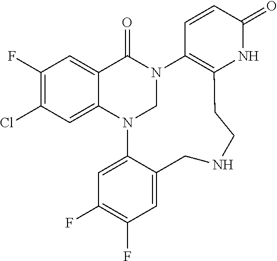 [SMILES: O=C1c2cc(F)c(Cl)cc2N2CN1c1ccc(=O)[nH]c1CCNCc1cc(F)c(F)cc12] | 3-Chloro-2,7,8-trifluoro-10,11,12,13-tetrahydro-19H-5,18- methanodibenzo[c,f]pyrido[2,3-j][1,5,9]triazocyclotridecine- 15,19(14H)-dione |
| 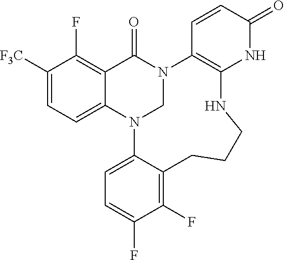 [SMILES: O=C1c2c(ccc(C(F)(F)F)c2F)N2CN1c1ccc(=O)[nH]c1NCCCc1c2ccc(F)c1F] | 1,8,9-Trifluoro-2-(trifluoromethyl)-10,11,12,13-tetrahydro-19H- 5,18-methanodibenzo[f,i]pyrido[2,3- b][1,4,8]triazocyclotridecine-15,19(14H)-dione |
| 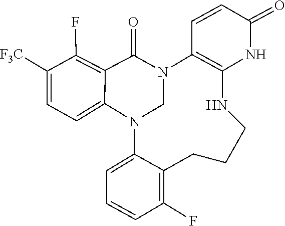 [SMILES: O=C1c2c(ccc(C(F)(F)F)c2F)N2CN1c1ccc(=O)[nH]c1NCCCc1c(F)cccc12] | 1,9-Difluoro-2-(trifluoromethyl)-10,11,12,13-tetrahydro-19H- 5,18-methanodibenzo[f,i]pyrido[2,3- b][1,4,8]triazocyclotridecine-15,19(14H)-dione |
| 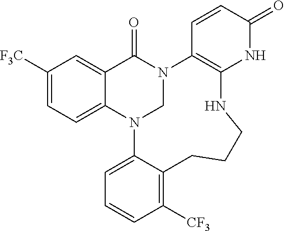 [SMILES: O=C1c2cc(C(F)(F)F)ccc2N2CN1c1ccc(=O)[nH]c1NCCCc1c2cccc1C(F)(F)F] | 2,9-bis(Trifluoromethyl)-10,11,12,13-tetrahydro-19H-5,18- methanodibenzo[f,i]pyrido[2,3-b][1,4,8]triazocyclotridecine- 15,19(14H)-dione |
| 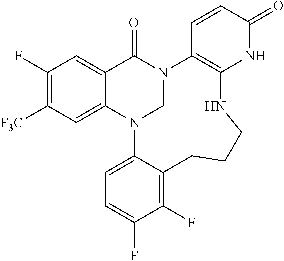 [SMILES: O=C1c2cc(F)c(C(F)(F)F)cc2N2CN1c1ccc(=O)[nH]c1NCCCc1c2ccc(F)c1F] | 2,8,9-Trifluoro-3-(trifluoromethyl)-10,11,12,13-tetrahydro-19H- 5,18-methanodibenzo[f,i]pyrido[2,3- b][1,4,8]triazocyclotridecine-15,19(14H)-dione |
| 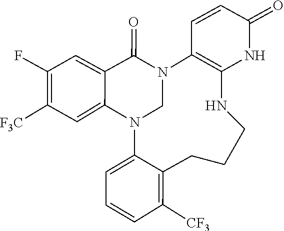 [SMILES: O=C1c2cc(F)c(C(F)(F)F)cc2N2CN1c1ccc(=O)[nH]c1NCCCc1c2cccc1C(F)(F)F] | 2-Fluoro-3,9-bis(trifluoromethyl)-10,11,12,13-tetrahydro-19H- 5,18-methanodibenzo[f,i]pyrido[2,3- b][1,4,8]triazocyclotridecine-15,19(14H)-dione |
| 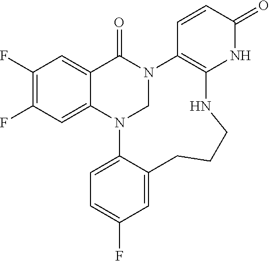 [SMILES: O=C1c2cc(F)c(F)cc2N2CN1c1ccc(=O)[nH]c1NCCCc1cc(F)ccc12] | 2,3,8-Trifluoro-10,11,12,13-tetrahydro-19H-5,18- methanodibenzo[f,i]pyrido[2,3-b][1,4,8]triazocyclotridecine- 15,19(14H)-dione |
| 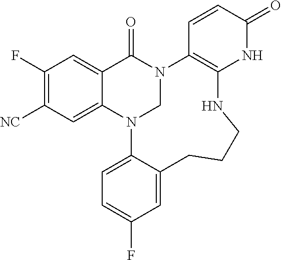 [SMILES: N#Cc1cc2c(cc1F)C(=O)N1CN2c2ccc(F)cc2CCCNc2[nH]c(=O)ccc21] | 2,8-Difluoro-15,19-dioxo-10,11,12,13-hexaahydro-19H-5,18- methanodibenzo[f,i]pyrido[2,3-b][1,4,8]triazocyclotridecine-3- carbonitrile |
| 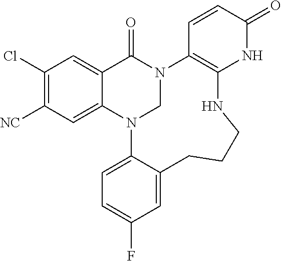 [SMILES: N#Cc1cc2c(cc1Cl)C(=O)N1CN2c2ccc(F)cc2CCCNc2[nH]c(=O)ccc21] | 2-Chloro-8-fluoro-15,19-dioxo-10,11,12,13-hexaahydro-19H- 5,18-methanodibenzo[f,/]pyrido[2,3- b][1,4,8]triazocyclotridecine-3-carbonitrile |
| 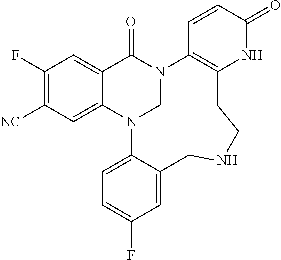 [SMILES: N#Cc1cc2c(cc1F)C(=O)N1CN2c2ccc(F)cc2CNCCc2[nH]c(=O)ccc21] | 2,8-Diifluoro-15,19-dioxo-10,11,12,13-hexahydro-19H-5,18- methanodibenzo[c,f]pyrido[2,3-j][1,5,9]triazocyclotridecine-3- carbonitrile |
| 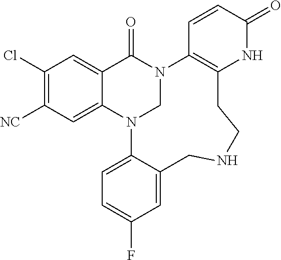 [SMILES: N#Cc1cc2c(cc1Cl)C(=O)N1CN2c2ccc(F)cc2CNCCc2[nH]c(=O)ccc21] | 2-Chloro-8-fluoro-15,19-dioxo-10,11,12,13-hexahydro-19H-5,18- methanodibenzo[c,f]pyrido[2,3-j][1,5,9]triazocyclotridecine-3- carbonitrile |
| 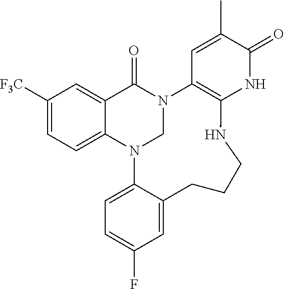 [SMILES: Cc1cc2c([nH]c1=O)NCCCc1cc(F)ccc1N1CN2C(=O)c2cc(C(F)(F)F)ccc21] | 8-Fluoro-16-methyl-2-(trifluoromethyl)-10,11,12,13-tetrahydro- 19H-5,18-methanodibenzo[f,i]pyrido[2,3- b][1,4,8]triazocyclotridecine-15,19(14H)-dione |
| 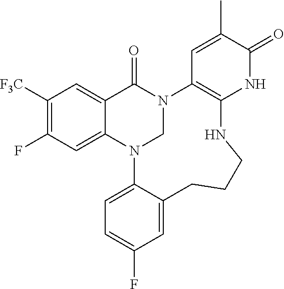 [SMILES: Cc1cc2c([nH]c1=O)NCCCc1cc(F)ccc1N1CN2C(=O)c2cc(C(F)(F)F)c(F)cc21] | 3,8-Difluoro-16-methyl-2-(trifluoromethyl)-10,11,12,13- tetrahydro-19H-5,18-methanodibenzo[f,i]pyrido[2,3- b][1,4,8]triazocyclotridecine-15,19(14H)-dione |
| 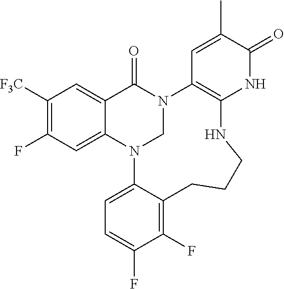 [SMILES: Cc1cc2c([nH]c1=O)NCCCc1c(ccc(F)c1F)N1CN2C(=O)c2cc(C(F)(F)F)c(F)cc21] | 3,8,9-Trifluoro-16-methyl-2-(trifluoromethyl)-10,11,12,13- tetrahydro-19H-5,18-methanodibenzo[f,i]pyrido[2,3- b][1,4,8]triazocyclotridecine-15,19(14H)-dione |
| 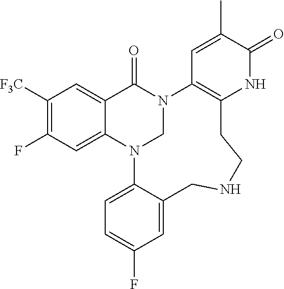 [SMILES: Cc1cc2c([nH]c1=O)CCNCc1cc(F)ccc1N1CN2C(=O)c2cc(C(F)(F)F)c(F)cc21] | 3,8-Difluoro-16-methyl-2-(trifluoromethyl)-10,11,12,13- tetrahydro-19H-5,18-methanodibenzo[c,f]pyrido[2,3- j][1,5,9]triazocyclotridecine-15,19(14H)-dione |
| 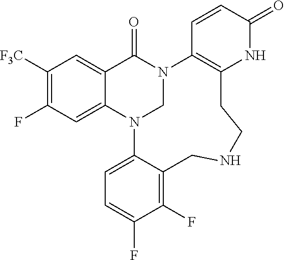 [SMILES: O=C1c2cc(C(F)(F)F)c(F)cc2N2CN1c1ccc(=O)[nH]c1CCNCc1c2ccc(F)c1F] | 3,8,9-Trifluoro-16-methyl-2-(trifluoromethyl)-10,11,12,13- tetrahydro-19H-5,18-methanodibenzo[c,f]pyrido[2,3- j][1,5,9]triazocyclotridecine-15,19(14H)-dione |
| 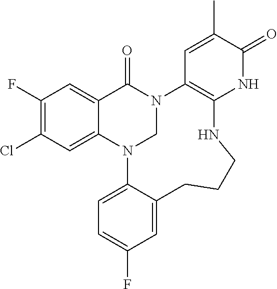 [SMILES: Cc1cc2c([nH]c1=O)NCCCc1cc(F)ccc1N1CN2C(=O)c2cc(F)c(Cl)cc21] | 3-Chloro-2,8-difluoro-16-methyl-10,11,12,13-tetrahydro-19H- 5,18-methanodibenzo[f,i]pyrido[2,3- b][1,4,8]triazocyclotridecine-15,19(14H)-dione |
| 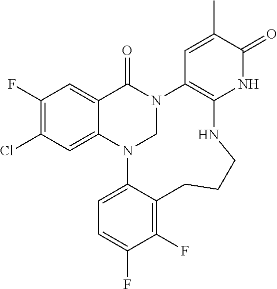 [SMILES: Cc1cc2c([nH]c1=O)NCCCc1c(ccc(F)c1F)N1CN2C(=O)c2cc(F)c(Cl)cc21] | 3-Chloro-2,8,9-trifluoro-16-methyl-10,11,12,13-tetrahydro-19H- 5,18-methanodibenzo[f,i]pyrido[2,3- b][1,4,8]triazocyclotridecine-15,19(14H)-dione |
| 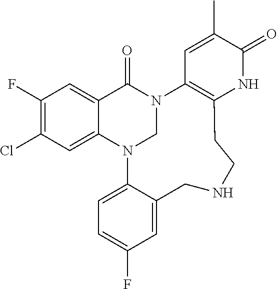 [SMILES: Cc1cc2c([nH]c1=O)CCNCc1cc(F)ccc1N1CN2C(=O)c2cc(F)c(Cl)cc21] | 3-Chloro-2,8-difluoro-16-methyl-10,11,12,13-tetrahydro-19H- 5,18-methanodibenzo[c,f]pyrido[2,3- j][1,5,9]triazocyclotridecine-15,19(14H)-dione |
| 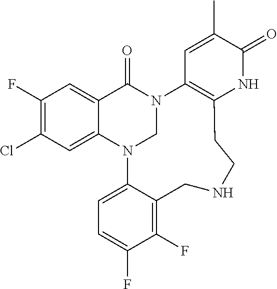 [SMILES: Cc1cc2c([nH]c1=O)CCNCc1c(ccc(F)c1F)N1CN2C(=O)c2cc(F)c(Cl)cc21] | 3-Chloro-2,8,9-trifluoro-16-methyl-10,11,12,13-tetrahydro-19H- 5,18-methanodibenzo[c,f]pyrido[2,3- j][1,5,9]triazocyclotridecine-15,19(14H)-dione |
| 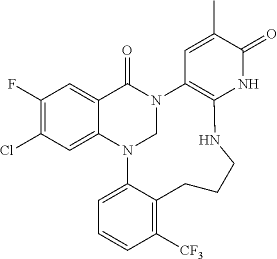 [SMILES: Cc1cc2c([nH]c1=O)NCCCc1c(cccc1C(F)(F)F)N1CN2C(=O)c2cc(F)c(Cl)cc21] | 3-Chloro-2-fluoro-16-methyl-9-(trifluoromethyl)-10,11,12,13- tetrahydro-19H-5,18-methanodibenzo[c,f]pyrido[2,3- j][1,5,9]triazocyclotridecine-15,19(14H)-dione |
| 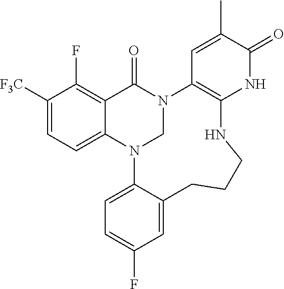 [SMILES: Cc1cc2c([nH]c1=O)NCCCc1cc(F)ccc1N1CN2C(=O)c2c1ccc(C(F)(F)F)c2F] | 2,8-Difluoro-16-methyl-2-(trifluoromethyl)-10,11,12,13- tetrahydro-19H-5,18-methanodibenzo[f,i]pyrido[2,3- b][1,4,8]triazocyclotridecine-15,19(14H)-dione |
| 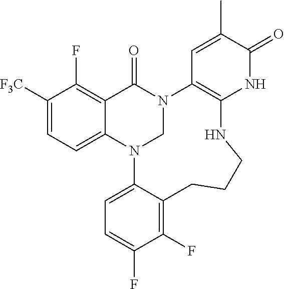 [SMILES: Cc1cc2c([nH]c1=O)NCCCc1c(ccc(F)c1F)N1CN2C(=O)c2c1ccc(C(F)(F)F)c2F] | 1,8,9-Trifluoro-16-methyl-2-(trifluoromethyl)-10,11,12,13- tetrahydro-19H-5,18-methanodibenzo[f,i]pyrido[2,3- b][1,4,8]triazocyclotridecine-15,19(14H)-dione |
| 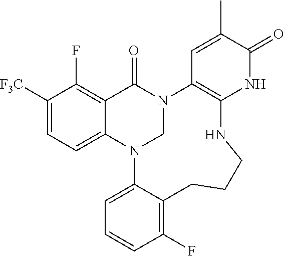 [SMILES: Cc1cc2c([nH]c1=O)NCCCc1c(F)cccc1N1CN2C(=O)c2c1ccc(C(F)(F)F)c2F] | 1,9-Difluoro-16-methyl-2-(trifluoromethyl)-10,11,12,13- tetrahydro-19H-5,18-methanodibenzo[f,i]pyrido[2,3- b][1,4,8]triazocyclotridecine-15,19(14H)-dione |
| 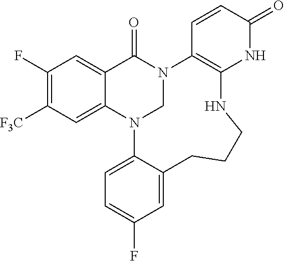 [SMILES: O=C1c2cc(F)c(C(F)(F)F)cc2N2CN1c1ccc(=O)[nH]c1NCCCc1cc(F)ccc12] | 2,8-Difluoro-16-methyl-3-(trifluoromethyl)-10,11,12,13- tetrahydro-19H-5,18-methanodibenzo[f,i]pyrido[2,3- b][1,4,8]triazocyclotridecine-15,19(14H)-dione |
| 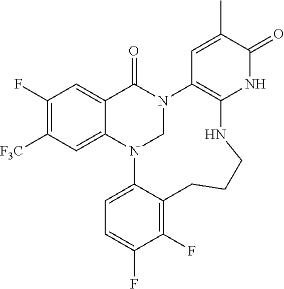 [SMILES: Cc1cc2c([nH]c1=O)NCCCc1c(ccc(F)c1F)N1CN2C(=O)c2cc(F)c(C(F)(F)F)cc21] | 2,8,9-Trifluoro-16-methyl-3-(trifluoromethyl)-10,11,12,13- tetrahydro-19H-5,18-methanodibenzo[f,i]pyrido[2,3- b][1,4,8]triazocyclotridecine-15,19(14H)-dione |
| 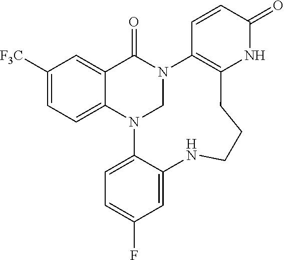 [SMILES: O=C1c2cc(C(F)(F)F)ccc2N2CN1c1ccc(=O)[nH]c1CCCNc1cc(F)ccc12] | 8-Fluoro-2-(trifluoromethyl)-10,11,12,13-tetrahydro-19H-5,18- methanodibenzo[b,e]pyrido[2,3-i][1,4,8]triazocyclotridecine- 15,19(14H)-dione |
| 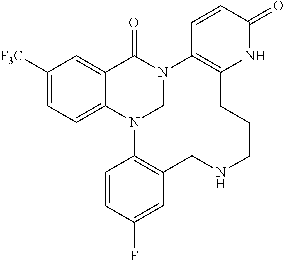 [SMILES: O=C1c2cc(C(F)(F)F)ccc2N2CN1c1ccc(=O)[nH]c1CCCNCc1cc(F)ccc12] | 3⁴-Fluoro-2⁶-(trifluoromethyl)-1¹,1²,2¹,2²,2³,2⁴-hexahydro-5-aza- 2(3,1)-quinazolina-1(5,6)-pyridina-3(1,2)- benzenacyclooctaphane-1²,2⁴-dione |
| 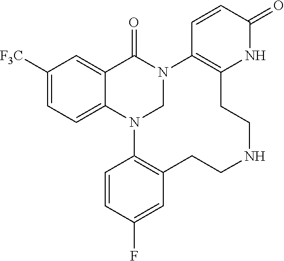 [SMILES: O=C1c2cc(C(F)(F)F)ccc2N2CN1c1ccc(=O)[nH]c1CCNCCc1cc(F)ccc12] | 3⁴-Fluoro-2⁶-(trifluoromethyl)-1¹,1²,2¹,2²,2³,2⁴-hexahydro-6-aza- 2(3,1)-quinazolina-1(5,6)-pyridina-3(1,2)- benzenacyclooctaphane-1²,2⁴-dione |
| 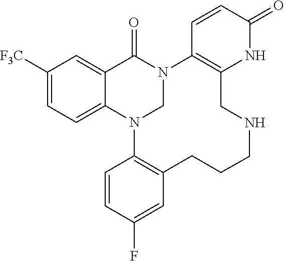 [SMILES: O=C1c2cc(C(F)(F)F)ccc2N2CN1c1ccc(=O)[nH]c1CNCCCc1cc(F)ccc12] | 3⁴-Fluoro-2⁶-(trifluoromethyl)-1¹,1²,2¹,2²,2³,2⁴-hexahydro-7-aza- 2(3,1)-quinazolina-1(5,6)-pyridina-3(1,2)- benzenacyclooctaphane-1²,2⁴-dione |
| 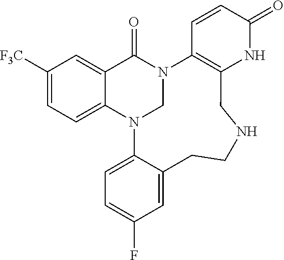 [SMILES: O=C1c2cc(C(F)(F)F)ccc2N2CN1c1ccc(=O)[nH]c1CNCCc1cc(F)ccc12] | 8-Fluoro-2-(trifluoromethyl)-10,11,12,13-tetrahydro-19H-5,18- methanodibenzo[b,l]pyrido[3,2-f][1,5,9]triazacyclotridecine- 15,19(14H)-dione |
| 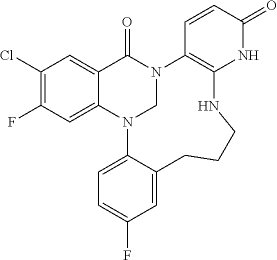 [SMILES: O=C1c2cc(Cl)c(F)cc2N2CN1c1ccc(=O)[nH]c1NCCCc1cc(F)ccc12] | 2-Chloro-3,8-difluoro-10,11,12,13-tetrahydro-19H-5,18- methanodibenzo[f,i]pyrido[2,3-b][1,4,8]triazacyclotridecine- 15,19(14H)-dione |
| 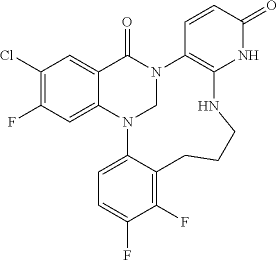 [SMILES: O=C1c2cc(Cl)c(F)cc2N2CN1c1ccc(=O)[nH]c1NCCCc1c2ccc(F)c1F] | 3-Chloro-3,8,9-trifluoro-10,11,12,13-tetrahydro-19H-5,18- methanodibenzo[f,i]pyrido[2,3-b][1,4,8]triazacyclotridecine- 15,19(14H)-dione |
| 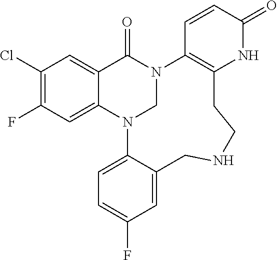 [SMILES: O=C1c2cc(Cl)c(F)cc2N2CN1c1ccc(=O)[nH]c1CCNCc1cc(F)ccc12] | 2-Chloro-3,8-difluoro-10,11,12,13-tetrahydro-19H-5,18- methanodibenzo[c,f]pyrido[3,2-j][1,5,9]triazacyclotridecine- 15,19(14H)-dione |
| 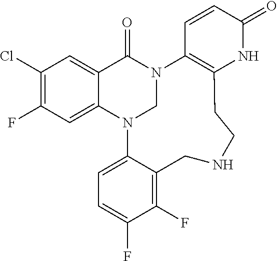 [SMILES: O=C1c2cc(Cl)c(F)cc2N2CN1c1ccc(=O)[nH]c1CCNCc1c2ccc(F)c1F] | 2-Chloro-3,8,9-trifluoro-10,11,12,13-tetrahydro-19H-5,18- methanodibenzo[c,f]pyrido[3,2-j][1,5,9]triazacyclotridecine- 15,19(14H)-dione |
| 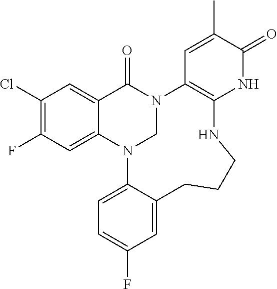 [SMILES: Cc1cc2c([nH]c1=O)NCCCc1cc(F)ccc1N1CN2C(=O)c2cc(Cl)c(F)cc21] | 2-Chloro-3,8-difluoro-16-methyl-10,11,12,13-tetrahydro-19H- 5,18-methanodibenzo[f,i]pyrido[2,3- b][1,4,8]triazacyclotridecine-15,19(14H)-dione |
| 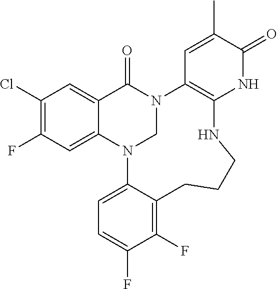 [SMILES: Cc1cc2c([nH]c1=O)NCCCc1c(ccc(F)c1F)N1CN2C(=O)c2cc(Cl)c(F)cc21] | 2-Chloro-3,8,9-trifluoro-16-methy-10,11,12,13-tetrahydro-19H- 5,18-methanodibenzo[f,i]pyrido[2,3- b][1,4,8]triazacyclotridecine-15,19(14H)-dione |
| 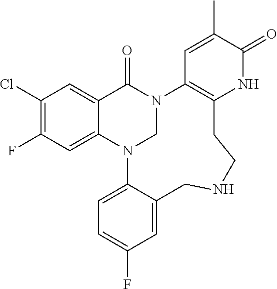 [SMILES: Cc1cc2c([nH]c1=O)CCNCc1cc(F)ccc1N1CN2C(=O)c2cc(Cl)c(F)cc21] | 2-Chloro-3,8-difluoro-16-methyl-10,11,12,13-tetrahydro-19H- 5,18-methanodibenzo[c,f]pyrido[3,2- j][1,5,9]triazacyclotridecine-15,19(14H)-dione |
| 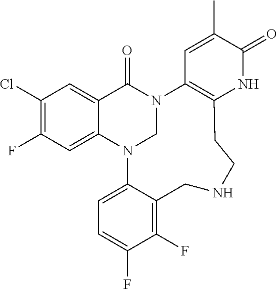 [SMILES: Cc1cc2c([nH]c1=O)CCNCc1c(ccc(F)c1F)N1CN2C(=O)c2cc(Cl)c(F)cc21] | 2-Chloro-3,8,9-trifluoro-16-methyl-10,11,12,13-tetrahydro-19H- 5,18-methanodibenzo[c,f]pyrido[3,2- j][1,5,9]triazacyclotridecine-15,19(14H)-dione |

### Biological Assays

### Biological assay Example 1

Human embryonic kidney 293 cells (HEK293) expressing human Naᵥ1.8, human Naᵥβ1 and human TREK1 (HEK293-Naᵥ1.8) were grown in T150 cell culture flasks at 37° C., 5% CO₂ incubator. HEK293-Naᵥ1.8 were passaged every 2-3 days when confluency reached 80-90% in T150 cell culture flasks.

Pharmacological assessment of compounds of the invention was performed using the QPatch 48 HTX electrophysiological platform. HEK293-Naᵥ1.8 cells were prepared on the day of use by removing culture media, washing in DPBS, adding Accutase (2 mL to cover the surface, aspirate 1 mL then 1-2 minutes at 37° C.) followed by addition of CHO-SFM II to stop the enzyme digestion and in order to obtain a suspension of 3×10⁶ cell/mL.

Compounds of the invention were prepared in an extracellular solution of the following composition: NaCl (145 mM), KCl (4 mM), CaCl₂) (2 mM), MgCl (2 mM), HEPES (1 mM), Glucose (10 mM), pH 7.4 with NaOH Osmolality 300 mOsM/L. An intracellular solution of the following composition was used: CsF (115 mM), CsCl (20 mM), NaCl (5 mM), EGTA (10 mM), HEPES (10 mM), Sucrose (20 mM), pH 7.2 with CsOH Osmolality 310 mOsm/L.

Utilizing the voltage-clamp mode in the QPatch 48 HTX system a half inactivation state voltage protocol (V_{1/2}) was used to determine pharmacological activity of compounds of the invention at Naᵥ1.8 ion channels. A V_{1/2} protocol was utilized with the following voltage steps: a holding voltage of −100 mV was established followed by a 20 ms voltage step to 0 mV (P1), followed by an inactivating voltage step at −46 mV for 8 seconds, followed by a step to −100 mV for 20 ms, before a 20 ms step to 0 mV (P2), then returned to the holding voltage of −100 mV. This voltage protocol was repeated at a frequency of every 15 seconds, the current magnitude was quantified at the P2 step throughout the recording. Inhibition of the measured current amplitude with compounds of the invention was analyzed by fitting a 4-6 points dose-response curve allowing determination of the fifty percent inhibition concentration (IC₅₀). P2 currents were normalized according to measurements made at baseline (Baseline, vehicle only), after compound (Input, at each test concentration), and after positive reference compound (FullResponse, to achieve complete block), fit to the following equation:

$$n . {I}_{CPD} = Normalized Current = \frac{( Input - Baseline )}{( FullResponse - Baseline )}$$

To assess current run-down over the course of the experiment vehicle-only wells were utilized and the normalized current with vehicle-only (n.I_{VEH}) was determined. To correct the compound response for run-down, the currents were corrected according to the following formula:

$$n . {I}_{RD \_ Correct} = \frac{( n . {I}_{CPD} - n . {I}_{VEH} )}{( 1 - n . {I}_{VEH} )}$$

Compound inhibition was fitted to the following Hill Equation to estimate the half maximal inhibition concentration (IC₅₀)

$$Y = \frac{{C}^{h}}{( {IC}_{50}^{h} + {C}^{h} )}$$

where Y is the normalized run-down corrected inhibition relative to the vehicle control (equivalent to n.I_{RD_Correct}), C the test compound concentration, IC₅₀ the concentration of test compound to inhibit the sodium current by 50%, and h the Hill coefficient.

The exemplified compounds of Examples 1-44 herein are active against Naᵥ1.8 sodium channels as measured using the assays described herein and as presented in Table 2 below.

Each of the listed compound Examples of the invention identified in the charts below, individually, was tested in at least one exemplified salt or free base form. Unless otherwise noted, the tested compound examples of the invention exhibited a pharmacological activity Naᵥ1.8 pIC₅₀ (Qpatch)>5.0. In another aspect, tested compound examples of the invention exhibited a pharmacological activity Naᵥ1.8 pIC₅₀ (Qpatch)>6.0.

TABLE 2
Biological Activity of Naᵥ1.8 Inhibitor Compounds

| | | | Nav1.8, |
|---|---|---|---|
| Example | Name | Structure | pIC₅₀ |
| 1 | (E)-8-Fluoro-2-(trifluoromethyl)-13-hydro- 10H, 18H, 5,17- methanodibenzo[b,k]pyrido[3,2- f][1,5]diazacyclododecine-14,18-dione | 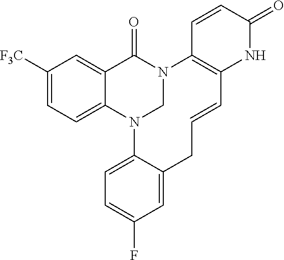 [SMILES: O=C1c2cc(C(F)(F)F)ccc2N2CN1c1ccc(=O)[nH]c1/C=C/Cc1cc(F)ccc12] | 5.1 |
| 2 | 14-Fluoro-8-(trifluoromethyl)-17,18-dihydro- 1H,6H-5,11-methanodibenzo[b,k]pyridino[3,2- f][1,5]diazacyclododecine-2,6(16H)-dione | 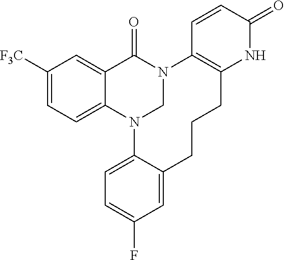 [SMILES: O=C1c2cc(C(F)(F)F)ccc2N2CN1c1ccc(=O)[nH]c1CCCc1cc(F)ccc12] | 6.0 |
| 3 | 8-Fluoro-2-(trifluoromethyl)-10,13-dihydro- 19H-5,18-methanodibenzo[b,l]pyrido[3,2- f][1,5]diazacyclotridecine-15,19(14H)-dione | 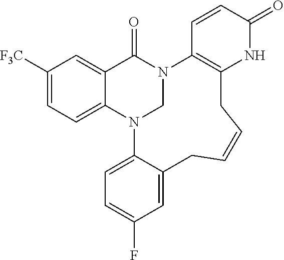 [SMILES: O=C1c2cc(C(F)(F)F)ccc2N2CN1c1ccc(=O)[nH]c1C/C=C\Cc1cc(F)ccc12] | 6.4 |
| | | + | |
| | | 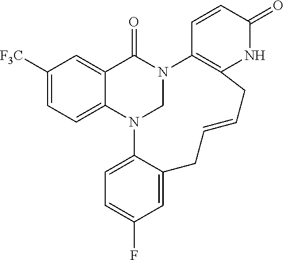 [SMILES: O=C1c2cc(C(F)(F)F)ccc2N2CN1c1ccc(=O)[nH]c1C/C=C/Cc1cc(F)ccc12] | |
| 4 | 8-Fluoro-2-(trifluoromethyl)-10,11,12,13- tetrahydro-19H-5,18- methanodibenzo[b,l]pyridino[3,2- f[1,5]diazacyclotridecine-15,19(14H)-dione | 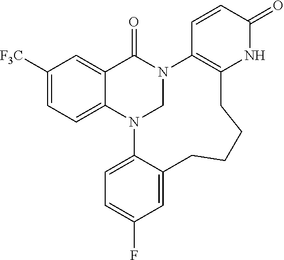 [SMILES: O=C1c2cc(C(F)(F)F)ccc2N2CN1c1ccc(=O)[nH]c1CCCCc1cc(F)ccc12] | 6.8 |
| 5 | 8-Fluoro-3-(trifluoromethyl)-10,13-dihydro- 19H-5,18-methanodibenzo[b,l]pyrido[3,2 f][1,5]diazacyclo tridecin-15,19(14H)-dion | 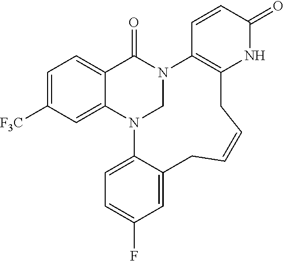 [SMILES: O=C1c2ccc(C(F)(F)F)cc2N2CN1c1ccc(=O)[nH]c1C/C=C\Cc1cc(F)ccc12] | 6.3 |
| | | + | |
| | | 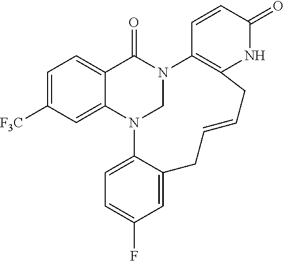 [SMILES: O=C1c2ccc(C(F)(F)F)cc2N2CN1c1ccc(=O)[nH]c1C/C=C/Cc1cc(F)ccc12] | |
| 6 | 8-Fluoro-3-(trifluoromethyl)-10,11,12,13- tetrahydro-19H-5,18-methanodibenzo[b,l] pyridino[3,2-f][1,5]diazacyclotridecine- 15,19(14H)-dione | 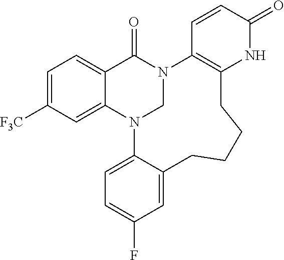 [SMILES: O=C1c2ccc(C(F)(F)F)cc2N2CN1c1ccc(=O)[nH]c1CCCCc1cc(F)ccc12] | 7.1 |
| 7 | 3⁴-Fluoro-2⁶-(trifluoromethyl)- 1¹,1²,1¹,2²,2³,2⁴-hexahydro-2(3,1)-qinazolina- 1(5,6)-pyridina-3(1,2)- benzenacyclooctaphane-1²,2⁴-dione | 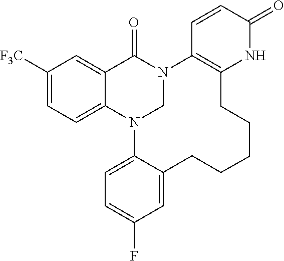 [SMILES: O=C1c2cc(C(F)(F)F)ccc2N2CN1c1ccc(=O)[nH]c1CCCCCc1cc(F)ccc12] | 8.1 |
| 8 | 3⁴-Fluoro-2⁷-(trifluoromethyl)- 1¹,1²,2¹,2²,2³,2⁴-hexahydro-2(3,1)-quinazolina- 1(5,6)-pyridina-3(1,2)- benzenacyclooctaphane-1²,2⁴-dione | 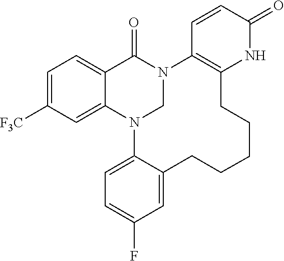 [SMILES: O=C1c2ccc(C(F)(F)F)cc2N2CN1c1ccc(=O)[nH]c1CCCCCc1cc(F)ccc12] | 8.4 |
| 9 | 3⁴-Fluoro-1⁶-methoxy-2⁷-(trifluoromethyl)- 2¹,2²,2³,2⁴-tetrahydro-2(3,1)-pyrido[4,3- d]pyrimidina-1(3,2)-pyridina-3(1,2)- benzenacyclooctaphane-2⁴-one | 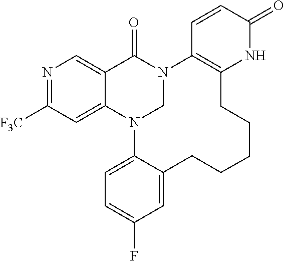 [SMILES: O=C1c2cnc(C(F)(F)F)cc2N2CN1c1ccc(=O)[nH]c1CCCCCc1cc(F)ccc12] | 7.3 |
| 10 | 3⁴-Fluoro-2⁶-(trifluoromethyl)- 1¹,1²,2¹,2²,2³,2⁴-hexahydro- 2(3,1)-quinazolina- 1(5,6)-pyridina-3(1,2)- benzenacyclononaphane-1²,2⁴-dione | 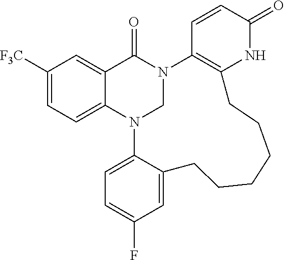 [SMILES: O=C1c2cc(C(F)(F)F)ccc2N2CN1c1ccc(=O)[nH]c1CCCCCCc1cc(F)ccc12] | 6.5 |
| 11 | 3⁴-Fluoro-2⁶-(trifluoromethyl)- 1¹,1²,2¹,2²,2³,2⁴-hexahydro-8-aza-2(3,1)- quinazolina-1(5,6)-pyridina-3(1,2)- benzenacyclooctaphane-1²,2⁴-dione | 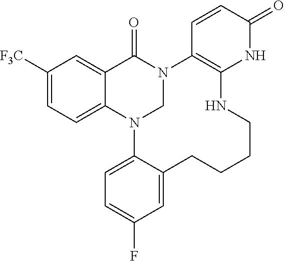 [SMILES: O=C1c2cc(C(F)(F)F)ccc2N2CN1c1ccc(=O)[nH]c1NCCCCc1cc(F)ccc12] | 7.3 |
| 12 | 3⁴-Fluoro-2⁷-(trifluoromethyl)- 1¹,1²,2¹,2²,2³,2⁴-hexahydro-8-aza-2(3,1)- quinazolina-1(5,6)-pyridina-3(1,2)- benzenacyclooctaphane-1²,2⁴-dione | 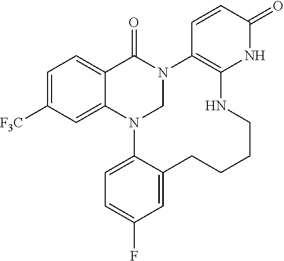 [SMILES: O=C1c2ccc(C(F)(F)F)cc2N2CN1c1ccc(=O)[nH]c1NCCCCc1cc(F)ccc12] | 7.6 |
| 13 | 3⁴-Fluoro-8-methyl-2⁷-(trifluoromethyl)- 1¹,1²,2¹,2²,2³,2⁴-hexahydro-8-aza-2(3,1)- quinazolina-1(5,6)-pyridina-3(1,2)- benzenacyclooctaphan-1²,2⁴-dione | 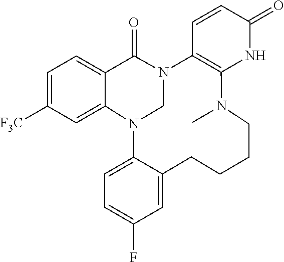 [SMILES: CN1CCCCc2cc(F)ccc2N2CN(C(=O)c3ccc(C(F)(F)F)cc32)c2ccc(=O)[nH]c21] | 7.3 |
| 14 | 3⁴-Fluoro-1³-iodo-8-methyl-2⁷- (trifluoromethyl)-1¹,1²,2¹,2²,2³,2⁴-hexahydro- 8-aza-2(3,1)-quinazolina-1(5,6)-pyridina- 3(1,2)-benzenacyclooctaphan-1²,2⁴-dione | 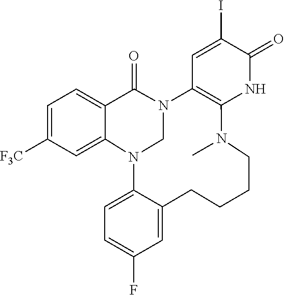 [SMILES: CN1CCCCc2cc(F)ccc2N2CN(C(=O)c3ccc(C(F)(F)F)cc32)c2cc(I)c(=O)[nH]c21] | 6.9 |
| 15 | 8-Fluoro-2-(trifluoromethyl)-1²,1³-dihydro- 11H,19H-5,18-methanodibenzo[i,j]pyrido[2,3- e][1]oxa[4,7,11]triazacyclotridecine- 15,19(14H)-dione | 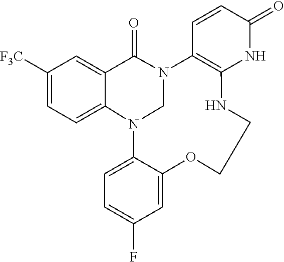 [SMILES: O=C1c2cc(C(F)(F)F)ccc2N2CN1c1ccc(=O)[nH]c1NCCOc1cc(F)ccc12] | 5.7 |
| 16 | 8-Fluoro2-(trifluoromethyl)-12,13-dihydro- 11H,19H-5,18- methanodibenzo[b,e]pyrido[3,2- i][1]oxa[4,8]diazacyclotridecin-15,19(14H)- dione | 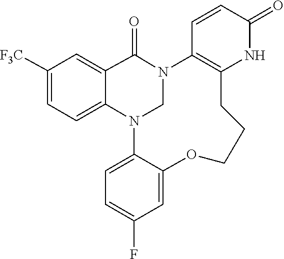 [SMILES: O=C1c2cc(C(F)(F)F)ccc2N2CN1c1ccc(=O)[nH]c1CCCOc1cc(F)ccc12] | 6.0 |
| 17 | 8-(Trifluoromethoxy)-2-(trifluoromethyl)- 10,11,12,13-tetahydro-19H,19H-5,18- methanodibenzo[l] pyrido[2,3-b:3′,2′- f[1,5]diazacyclotridecine-15,19(14H)-dione | 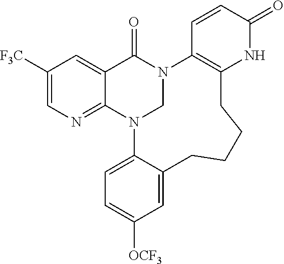 [SMILES: O=C1c2cc(C(F)(F)F)cnc2N2CN1c1ccc(=O)[nH]c1CCCCc1cc(OC(F)(F)F)ccc12] | 6.1 |
| 18 | 3⁴-Fluoro-2⁷-(trifluoromethyl)- 1¹,1²,2¹,2²,2³,2⁴-hexahydro-4-oxa-2(3,1)- quinazolina-1(5,6)-pyridina-3(1,2)- benzenacyclooctaphane-1²,2⁴-dione | 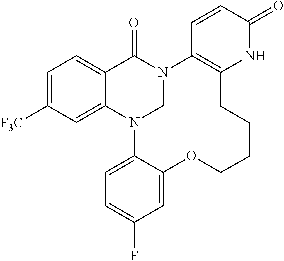 [SMILES: O=C1c2ccc(C(F)(F)F)cc2N2CN1c1ccc(=O)[nH]c1CCCCOc1cc(F)ccc12] | 6.5 |
| 19 | 3⁴-Fluoro-2⁷-(trifluoromethyl)- 1¹, 1²,2¹,2²,2³,2⁴-hexahydro-4-oxa-2(3,1)- quinazolina-1(5,6)-pyridina-3(1,2)- benzenacyclonaphane-1²,2⁴-dione | 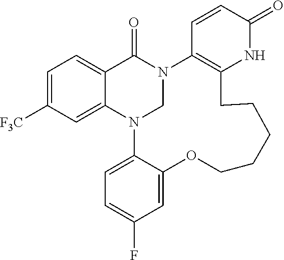 [SMILES: O=C1c2ccc(C(F)(F)F)cc2N2CN1c1ccc(=O)[nH]c1CCCCCOc1cc(F)ccc12] | 6.5 |
| 20 | 2⁶-chloro-3⁴-fluoro-1¹,1²,2¹,2²,2³,2⁴- hexahydro-2(3,1)-pyrido[2,3-d]pyrimidina- 1(5,6)-pyridina-3(1,2)- benzenacyclooctaphane-1²,2⁴-dione | 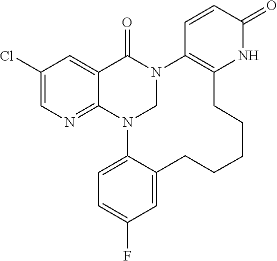 [SMILES: O=C1c2cc(Cl)cnc2N2CN1c1ccc(=O)[nH]c1CCCCCc1cc(F)ccc12] | 7.2 |
| 21 | 3⁴-Fluoro-2⁶-(trifluoromethyl)- 1¹,1²,2¹,2²,2³,2⁴-hexahydro- 2(3,1)-pyrido[3,4- d]pyrimidina-1(5,6)-pyridina-3(1,2)- benzenacyclooctaphane-1²,2⁴-dione | 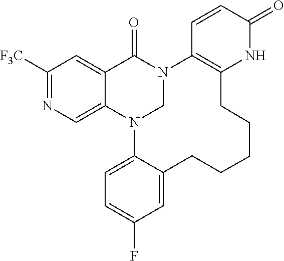 [SMILES: O=C1c2cc(C(F)(F)F)ncc2N2CN1c1ccc(=O)[nH]c1CCCCCc1cc(F)ccc12] | 7.6 |
| 22 | 3⁴-Fluoro-2⁷-(trifluoromethyl)- 1¹,1²,2¹,2²,2³,2⁴-hexahydro-2(3,1)-pyrido[2,3- d]pyrimidina-1(5,6)-pyridina-3(1,2)- benzenacyclooctaphane-1²,2⁴-dione | 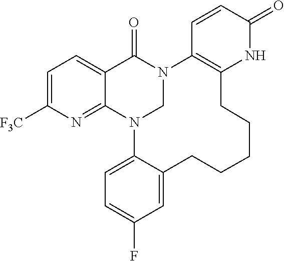 [SMILES: O=C1c2ccc(C(F)(F)F)nc2N2CN1c1ccc(=O)[nH]c1CCCCCc1cc(F)ccc12] | 7.6 |
| 23 | 3⁴-Fluoro-2⁶-(trifluoromethyl)- 1¹,1²,2¹,2²,2³,2⁴-hexahydro-2(3,1)-pyrido[2,3- d]pyrimidina-1(5,6)-pyridina-3(1,2)- benzenacyclooctaphane-1²,2⁴-dione | 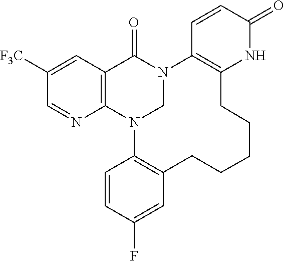 [SMILES: O=C1c2cc(C(F)(F)F)cnc2N2CN1c1ccc(=O)[nH]c1CCCCCc1cc(F)ccc12] | 7.2 |
| 24 | 3⁴-Fluoro-2⁶-(trifluoromethyl)- 1¹,1²,2¹,2²,2³,2⁴-hexahydro-2(3,1)-pyrido[2,3- d]pyrimidina-1(5,6)-pyridina-3(1,2)- benzenacyclooctaphan-5-ene-1²,2⁴-dione | 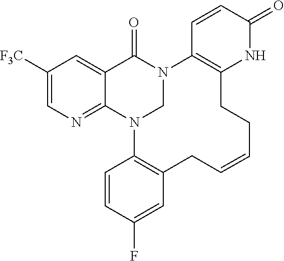 [SMILES: O=C1c2cc(C(F)(F)F)cnc2N2CN1c1ccc(=O)[nH]c1CC/C=C\Cc1cc(F)ccc12] | 7.7 |
| | | 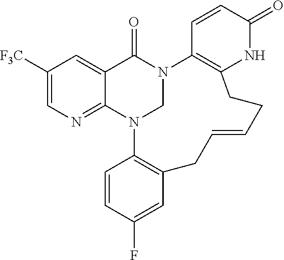 [SMILES: O=C1c2cc(C(F)(F)F)cnc2N2CN1c1ccc(=O)[nH]c1CC/C=C/Cc1cc(F)ccc12] | |
| 25 | 3⁴-Fluoro-2⁶-(trifluoromethyl)- 1¹,1²,2¹,2²,2³,2⁴-hexahydro-4-aza-2(3,1)- quinazolina-1(5,6)-pyridina-3(1,2)- benzenacyclooctaphan-1²,2⁴-dione | 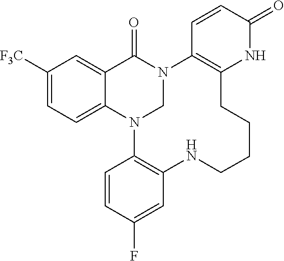 [SMILES: O=C1c2cc(C(F)(F)F)ccc2N2CN1c1ccc(=O)[nH]c1CCCCNc1cc(F)ccc12] | 6.2 |
| 26 | 3⁴-Fluoro-2⁴-oxo-2⁶-(trifluoromethyl)- 2¹,2²,,2³,2⁴-tetrahydro-2(3,1)-quinazolina- 1(4,3)-pyridin-1-iuma-3(1,2)- benzenacyclooctaphan-1¹-oxide | 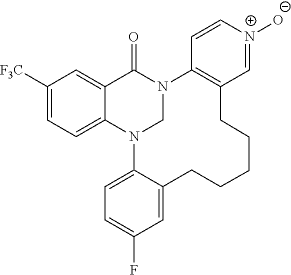 [SMILES: O=C1c2cc(C(F)(F)F)ccc2N2CN1c1cc[n+]([O-])cc1CCCCCc1cc(F)ccc12] | 8.3 |
| 27 | 8-Fluoro-2-(trifluoromethyl)-10,11,12,13- tetrahydro-19H-5,18- methanodibenzo[f,i]pyrido[2,3- b][1,4,8]triazocyclotridecine-15,19(14H)-dione | 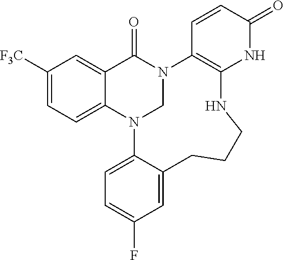 [SMILES: O=C1c2cc(C(F)(F)F)ccc2N2CN1c1ccc(=O)[nH]c1NCCCc1cc(F)ccc12] | 7.8 |
| 28 | 8-Fluoro-3-(trifluoromethyl)-10,11,12,13- tetrahydro-19H-5,18- methanodibenzo[f,i]pyrido[2,3- b][1,4,8]triazocyclotridecine-15,19(14H)-dione | 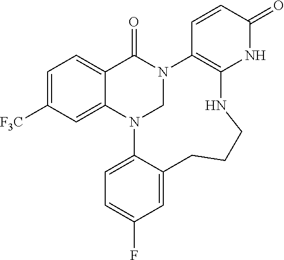 [SMILES: O=C1c2ccc(C(F)(F)F)cc2N2CN1c1ccc(=O)[nH]c1NCCCc1cc(F)ccc12] | 7.5 |
| 29 | 16-Chloro-8-fluoro-2-(trifluoromethyl)- 10,11,12,13-tetrahydro-19H-5,18- methanodibenzo[f,i] pyrido[2,3,- b][1,4,8]triazacyclotridecine-15,19(14H)-dione | 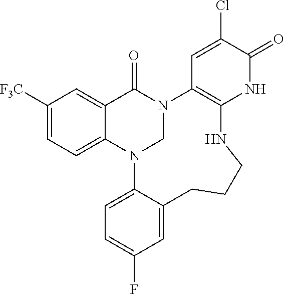 [SMILES: O=C1c2cc(C(F)(F)F)ccc2N2CN1c1cc(Cl)c(=O)[nH]c1NCCCc1cc(F)ccc12] | 7.1 |
| 30 | 8-Fluoro-13-methyl-2-(trifluoromethyl)- 10,11,12,13-tetrahydro-19H-5,18- methanodibenzo[f,i] pyrido[2,3,- b][1,4,8]triazacyclotridecine-15,19(14H)-dione | 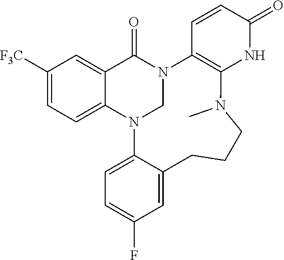 [SMILES: CN1CCCc2cc(F)ccc2N2CN(C(=O)c3cc(C(F)(F)F)ccc32)c2ccc(=O)[nH]c21] | 6.9 |
| 31 | 8-Fluoro-2-(trifluoromethoxy)-10,11,12,13- tetrahydro-19H-5,18- methanodibenzo[f,i]pyrido[2,3,- b][1,4,8]triazacyclotridecine-15,19(14H)-dione | 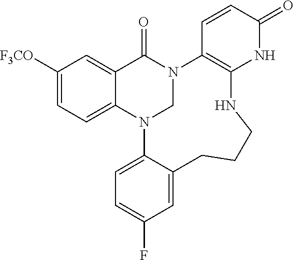 [SMILES: O=C1c2cc(OC(F)(F)F)ccc2N2CN1c1ccc(=O)[nH]c1NCCCc1cc(F)ccc12] | 7.5 |
| 32 | 2,8-Difluoro-3-(trifluoromethoxy)- 10,11,12,13-tetrahydro-19H-5,18- methanodibenzo[f,i]pyrido[2,3,- b][1,4,8]triazacyclotridecine-15,19(14H)-dione | 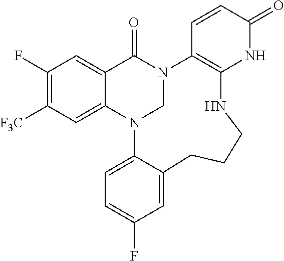 [SMILES: O=C1c2cc(F)c(C(F)(F)F)cc2N2CN1c1ccc(=O)[nH]c1NCCCc1cc(F)ccc12] | 8.4 |
| 33 | 1,8-Difluoro-2-(trifluoromethyl)-10,11,12,13- tetrahydro-19H-5,18- methanodibenzo[f,i]pyrido[2,3,- b][1,4,8]triazacyclotridecine-15,19(14H)-dione | 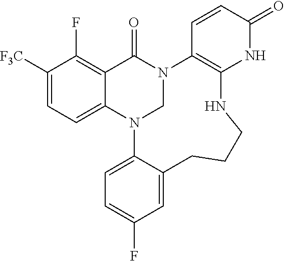 [SMILES: O=C1c2c(ccc(C(F)(F)F)c2F)N2CN1c1ccc(=O)[nH]c1NCCCc1cc(F)ccc12] | 7.5 |
| 34 | 8-Fluoro-2-(trifluoromethyl)-10,11,12,13- tetrahydro-19H-5,18- methanodibenzo[i]pyrido[2,3,-b:3′,2′- f][1,4,8]triazacyclotridecine-15,19(14H)-dione | 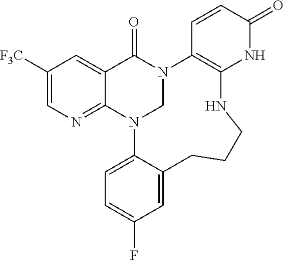 [SMILES: O=C1c2cc(C(F)(F)F)cnc2N2CN1c1ccc(=O)[nH]c1NCCCc1cc(F)ccc12] | 6.4 |
| 35 | 8-Fluoro-2-(trifluoromethyl)-10,11,12,13- tetrahydro-19H-5,18- methanodibenzo[i]dipyrido[2,3,-b:4′,3′- f][1,4,8]triazacyclotridecine-15,19(14H)-dione | 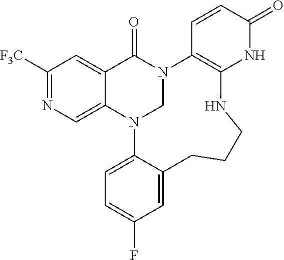 [SMILES: O=C1c2cc(C(F)(F)F)ncc2N2CN1c1ccc(=O)[nH]c1NCCCc1cc(F)ccc12] | 6.9 |
| 36 | 8-Fluoro-3-(trifluoromethyl)-10,11,12,13- tetrahydro-19H-5,18- methanodibenzo[i]dipyrido[2,3,-b:3′,4′- f][1,4,8]triazacyclotridecine-15,19(14H)-dione | 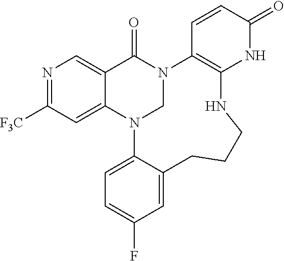 [SMILES: O=C1c2cnc(C(F)(F)F)cc2N2CN1c1ccc(=O)[nH]c1NCCCc1cc(F)ccc12] | 6.6 |
| 37 | 8,9-Difluoro-2-(trifluoromethyl)-10,11,12,13- tetrahydro-19H-5,18- methanodibenzo[f,i]dipyrido[2,3,- b][1,4,8]triazacyclotridecine-15,19(14H)-dione | 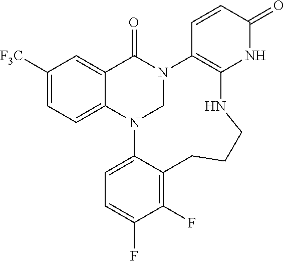 [SMILES: O=C1c2cc(C(F)(F)F)ccc2N2CN1c1ccc(=O)[nH]c1NCCCc1c2ccc(F)c1F] | 7.7 |
| 38 | 8,9-Diluoro-2-(trifluoromethyl)-10,11,12,13- tetrahydro-19H-5,18- methanodibenzo[i]dipyrido[2,3,-b:4′,3′- f][1,4,8]triazacyclotridecine-15,19(14H)-dione | 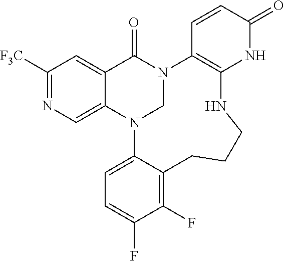 [SMILES: O=C1c2cc(C(F)(F)F)ncc2N2CN1c1ccc(=O)[nH]c1NCCCc1c2ccc(F)c1F] | 6.8 |
| 39 | 7,8-Difluoro-2-(trifluoromethyl)-10,11,12,13- tetrahydro-19H-5,18- methanodibenzo[f,i]dipyrido[2,3,- b][1,4,8]triazacyclotridecine-15,19(14H)-dione | 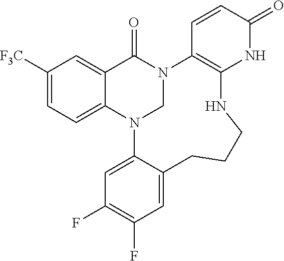 [SMILES: O=C1c2cc(C(F)(F)F)ccc2N2CN1c1ccc(=O)[nH]c1NCCCc1cc(F)c(F)cc12] | 7.0 |
| 40 | 8-Fluoro-2-(trifluoromethyl)-10,11,12,13- tetrahydro-19H-5,18- methanobenzo[/]dipyrido[3,4-b:3′,2′- f][1,5]diazocyclotridecine-15,19(14H)-dione | 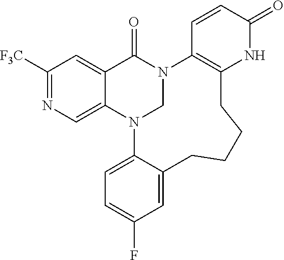 [SMILES: O=C1c2cc(C(F)(F)F)ncc2N2CN1c1ccc(=O)[nH]c1CCCCc1cc(F)ccc12] | 6.0 |
| 41 | 8-Fluoro-2-(trifluoromethyl)-10,11,12,13- tetrahydro-19H-5,18- methanodibenzo[c,f]pyrido[3,2- j][1,5,9]triazacyclotridecine-15,19(14H)-dione | 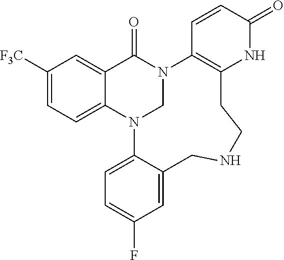 [SMILES: O=C1c2cc(C(F)(F)F)ccc2N2CN1c1ccc(=O)[nH]c1CCNCc1cc(F)ccc12] | 7.3 |
| 42 | 2-Chloro-8-fluoro-10,11,12,13-tetrahydro- 19H-5,18-methanodibenzo[c]dipyrido[2,3- f:3′,2′-j][1,5,9]triazacyclotridecine- 15,19(14H)-dione | 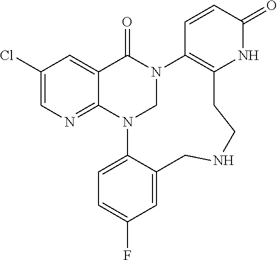 [SMILES: O=C1c2cc(Cl)cnc2N2CN1c1ccc(=O)[nH]c1CCNCc1cc(F)ccc12] | 6.9 |
| 43 | 2-Chloro-3,8-difluoro-10,11,12,13-tetrahydro- 19H-5,18-methanodibenzo[c,f]pyrido[3,2- j][1,5,9]triazacyclotridecine-15,19(14H)-dione | 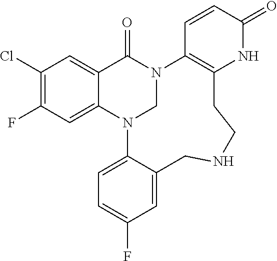 [SMILES: O=C1c2cc(Cl)c(F)cc2N2CN1c1ccc(=O)[nH]c1CCNCc1cc(F)ccc12] | 7.4 |
| 44 | 2,8-Difluoro-3-(trifluoromethyl)-10,11,12,13- tetrahydro-19H-5,18- methanodibenzo[c,f]pyrido[3,2- j][1,5,9]triazacyclotridecine-15,19(14H)-dione | 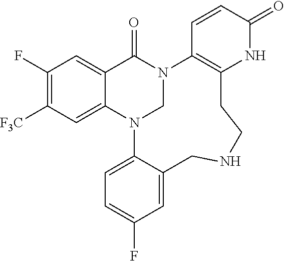 [SMILES: O=C1c2cc(F)c(C(F)(F)F)cc2N2CN1c1ccc(=O)[nH]c1CCNCc1cc(F)ccc12] | 8.6 |
| 45 | 1,8-Difluoro-2-(trifluoromethyl)-10,11,12,13- tetrahydro-19H-5,18- methanodibenzo[c,f]pyrido[3,2- j][1,5,9]triazacyclotridecine-15,19(14H)-dione | 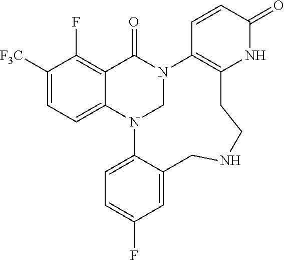 [SMILES: O=C1c2c(ccc(C(F)(F)F)c2F)N2CN1c1ccc(=O)[nH]c1CCNCc1cc(F)ccc12] | 6.9 |
| 46 | 3-Chloro-2,8-difluoro-10,11,12,13-tetrahydro- 19H-5,18-methanodibenzo[c,f]pyrido[3,2-j] [1,5,9]triazacyclotridecine-15,19(14H)-dione, hydrochloride | 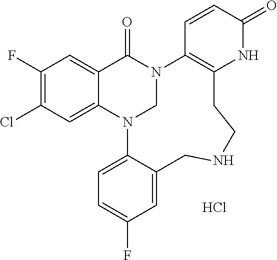 [SMILES: O=C1c2cc(F)c(Cl)cc2N2CN1c1ccc(=O)[nH]c1CCNCc1cc(F)ccc12 ; Cl] | 8 |
| 47 | 7,8-Difluoro-2-(trifluoromethyl)-10,11,12,13- tetrahydro-19H-5,18- methanodibenzo[c,f]pyrido[3,2-j] [1,5,9]triazacyclotridecine-15,19(14H)-dione hydrochloride | 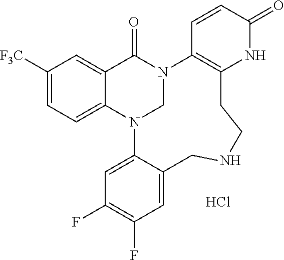 [SMILES: O=C1c2cc(C(F)(F)F)ccc2N2CN1c1ccc(=O)[nH]c1CCNCc1cc(F)c(F)cc12 ; Cl] | 6.7 |
| 48 | 2,8,9-Trifluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 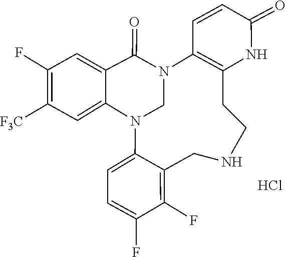 [SMILES: O=C1c2cc(F)c(C(F)(F)F)cc2N2CN1c1ccc(=O)[nH]c1CCNCc1c2ccc(F)c1F ; Cl] | 8.1 |
| 49 | 3-Chloro-2,8,9-trifluoro-10,11,12,13- tetrahydro-19H-5,18- methanodibenzo[c,f]pyrido[3,2-j] [1,5,9]triazacyclotridecine-15,19(14H)-dione hydrochloride | 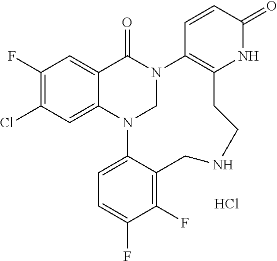 [SMILES: O=C1c2cc(F)c(Cl)cc2N2CN1c1ccc(=O)[nH]c1CCNCc1c2ccc(F)c1F ; Cl] | 8.2 |
| 50 | 3-Chloro-2,8,9-trifluoro-10,11,12,13- tetrahydro-19H-5,18- methanodibenzo[c,f]pyrido[3,2-j] [1,5,9]triazacyclotridecine-15,19(14H)-dione | 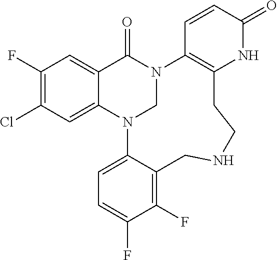 [SMILES: O=C1c2cc(F)c(Cl)cc2N2CN1c1ccc(=O)[nH]c1CCNCc1c2ccc(F)c1F] | 8 |
| 51 | 2-Fluoro-8-(trifluoromethoxy)-3- (trifluoromethyl)-10,11,12,13-tetrahydro-19H- 5,18-methanodibenzo[c,f]pyrido[3,2-j] [1,5,9]triazacyclotridecine-15,19(14H)-dione hydrochloride | 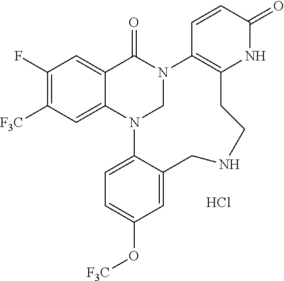 [SMILES: O=C1c2cc(F)c(C(F)(F)F)cc2N2CN1c1ccc(=O)[nH]c1CCNCc1cc(OC(F)(F)F)ccc12 ; Cl] | 8 |
| 52 | 2,9-Difluoro-8-(trifluoromethoxy)-3- (trifluoromethyl)-10,11,12,13-tetrahydro-19H- 5,18-methanodibenzo[c,f]pyrido[3,2-j] [1,5,9]triazacyclotridecine-15,19(14H)-dione hydrochloride | 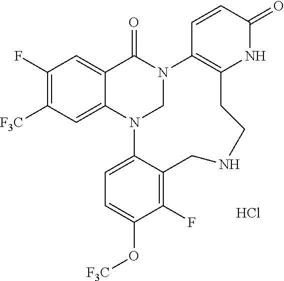 [SMILES: O=C1c2cc(F)c(C(F)(F)F)cc2N2CN1c1ccc(=O)[nH]c1CCNCc1c2ccc(OC(F)(F)F)c1F ; Cl] | 8.5 |
| 53 | 2,3,8-Trifluoro-10,11,12,13-tetrahydro-19H- 5,18-methanodibenzo[c,f]pyrido[3,2-j] [1,5,9]triazacyclotridecine-15,19(14H)-dione hydrochloride | 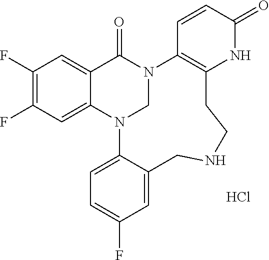 [SMILES: O=C1c2cc(F)c(F)cc2N2CN1c1ccc(=O)[nH]c1CCNCc1cc(F)ccc12 ; Cl] | 6.7 |
| 54 | 2,8-Difluoro-3-(methylamino)-10,11,12,13- tetrahydro-19H-5,18- methanodibenzo[c,f]pyrido[3,2-j] [1,5,9]triazacyclotridecine-15,19(14H)-dione | 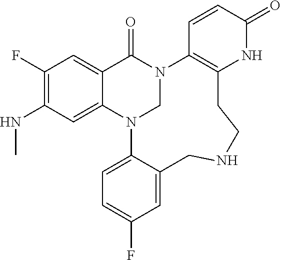 [SMILES: CNc1cc2c(cc1F)C(=O)N1CN2c2ccc(F)cc2CNCCc2[nH]c(=O)ccc21] | 6 |
| 55 | 2-Chloro-8-fluoro-15,19-dioxo- 10,11,12,13,14,15-hexahydro-19H-5,18- methanodibenzo[c,f]pyrido[3,2-j] [1,5,9]triazacyclotridecine-3-carbonitrile | 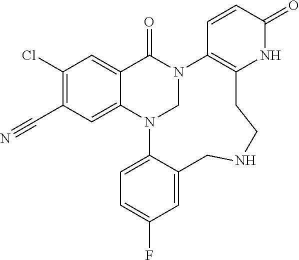 [SMILES: N#Cc1cc2c(cc1Cl)C(=O)N1CN2c2ccc(F)cc2CNCCc2[nH]c(=O)ccc21] | 7.2 |
| 56 | 8-Fluoro-2-(2,2,2-trifluoroethoxy)- 10,11,12,13-tetrahydro-19H-5,18- methanodibenzo[c,f]pyrido[3,2-j] [1,5,9]triazacyclotridecine-15,19(14H)-dione hydrochloride | 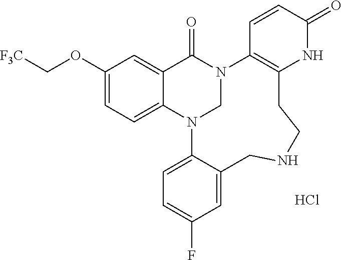 [SMILES: O=C1c2cc(OCC(F)(F)F)ccc2N2CN1c1ccc(=O)[nH]c1CCNCc1cc(F)ccc12 ; Cl] | 5.9 |
| 57 | 2,8,9-Trifluoro-15,19-dioxo- 10,11,12,13,14,15-hexahydro-19H-5,18- methanodibenzo[c,f]pyrido[3,2-j] [1,5,9]triazacyclotridecine-3-carbonitrile | 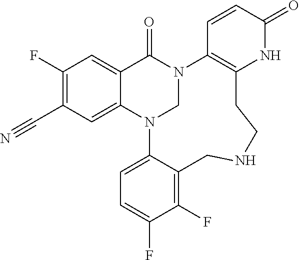 [SMILES: N#Cc1cc2c(cc1F)C(=O)N1CN2c2ccc(F)c(F)c2CNCCc2[nH]c(=O)ccc21] | 7.4 |
| 58 | 9-Chloro-2,8-difluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione | 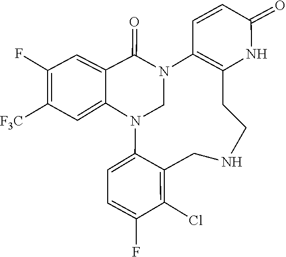 [SMILES: O=C1c2cc(F)c(C(F)(F)F)cc2N2CN1c1ccc(=O)[nH]c1CCNCc1c2ccc(F)c1Cl] | 8.9 |
| 59 | 2-Chloro-8-fluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 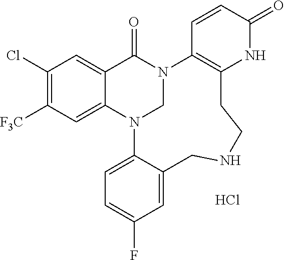 [SMILES: O=C1c2cc(Cl)c(C(F)(F)F)cc2N2CN1c1ccc(=O)[nH]c1CCNCc1cc(F)ccc12 ; Cl] | 7.8 |
| 60 | 2-Chloro-8,9-difluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 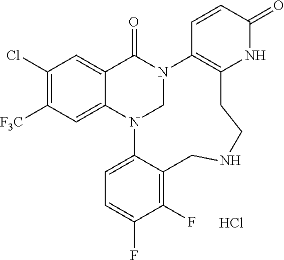 [SMILES: O=C1c2cc(Cl)c(C(F)(F)F)cc2N2CN1c1ccc(=O)[nH]c1CCNCc1c2ccc(F)c1F ; Cl] | 8.8 |
| 61 | 2,8-Difluoro-9-methyl-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 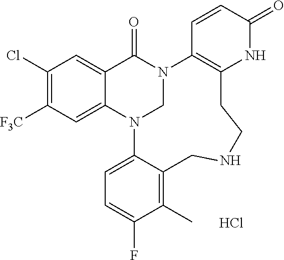 [SMILES: Cc1c(F)ccc2c1CNCCc1[nH]c(=O)ccc1N1CN2c2cc(C(F)(F)F)c(Cl)cc2C1=O ; Cl] | 8.4 |
| 62 | 3-Bromo-2,8,9-trifluoro-10,11,12,13- tetrahydro-19H-5,18- methanodibenzo[c,f]pyrido[3,2-j] [1,5,9]triazacyclotridecine-15,19(14H)-dione hydrochloride | 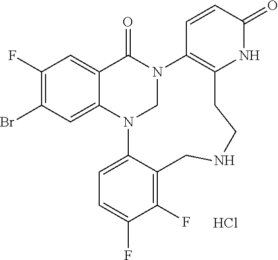 [SMILES: O=C1c2cc(F)c(Br)cc2N2CN1c1ccc(=O)[nH]c1CCNCc1c2ccc(F)c1F ; Cl] | 8.2 |
| 63 | 3-Bromo-2,8-difluoro-10,11,12,13-tetrahydro- 19H-5,18-methanodibenzo[c,f]pyrido[3,2-j] [1,5,9]triazacyclotridecine-15,19(14H)-dione hydrochloride | 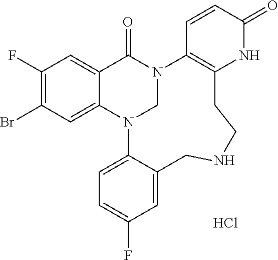 [SMILES: O=C1c2cc(F)c(Br)cc2N2CN1c1ccc(=O)[nH]c1CCNCc1cc(F)ccc12 ; Cl] | 8 |
| 64 | 9-Chloro-2,3,8-trifluoro-10,11,12,13- tetrahydro-19H-5,18- methanodibenzo[c,f]pyrido[3,2-j] [1,5,9]triazacyclotridecine-15,19(14H)-dione hydrochloride | 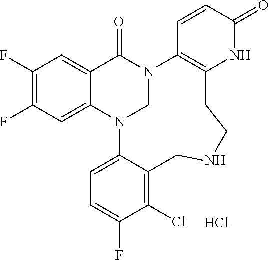 [SMILES: O=C1c2cc(F)c(F)cc2N2CN1c1ccc(=O)[nH]c1CCNCc1c2ccc(F)c1Cl ; Cl] | 8.2 |
| 65 | 9-Chloro-2,3,8-trifluoro-10,11,12,13- tetrahydro-19H-5,18- methanodibenzo[c,f]pyrido[3,2-j] [1,5,9]triazacyclotridecine-15,19(14H)-dione- 20,20-d2 hydrochloride | 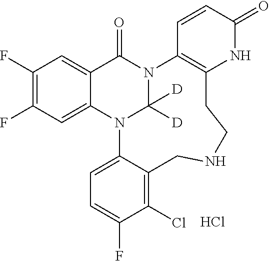 [SMILES: [2H]C1([2H])N2C(=O)c3cc(F)c(F)cc3N1c1ccc(F)c(Cl)c1CNCCc1[nH]c(=O)ccc12 ; Cl] | 8.3 |
| 66 | 8-Chloro-2-fluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 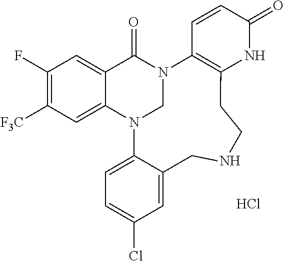 [SMILES: O=C1c2cc(F)c(C(F)(F)F)cc2N2CN1c1ccc(=O)[nH]c1CCNCc1cc(Cl)ccc12 ; Cl] | 8.2 |
| 67 | 9-Chloro-2,8-difluoro-15,19-dioxo- 10,11,12,13,14,15-hexahydro-19H-5,18- methanodibenzo[c,f]pyrido[3,2-j] [1,5,9]triazacyclotridecine-3-carbonitrile | 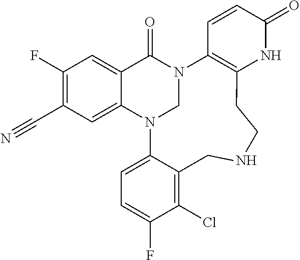 [SMILES: N#Cc1cc2c(cc1F)C(=O)N1CN2c2ccc(F)c(Cl)c2CNCCc2[nH]c(=O)ccc21] | 8.3 |
| 68 | 2,3,8-Trifluoro-9-methyl-10,11,12,13- tetrahydro-19H-5,18- methanodibenzo[c,f]pyrido[3,2-j] [1,5,9]triazacyclotridecine-15,19(14H)-dione hydrochloride | 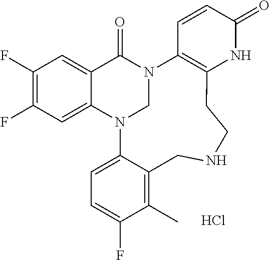 [SMILES: Cc1c(F)ccc2c1CNCCc1[nH]c(=O)ccc1N1CN2c2cc(F)c(F)cc2C1=O ; Cl] | 7.7 |
| 69 | 2-Chloro-3,8,9-trifluoro-10,11,12,13- tetrahydro-19H-5,18- methanodibenzo[c,f]pyrido[3,2-j] [1,5,9]triazacyclotridecine-15,19(14H)-dione hydrochloride | 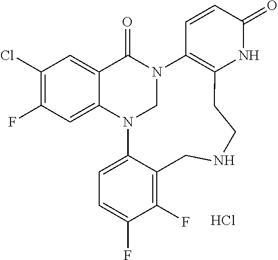 [SMILES: O=C1c2cc(Cl)c(F)cc2N2CN1c1ccc(=O)[nH]c1CCNCc1c2ccc(F)c1F ; Cl] | 7.6 |
| 70 | 2,3,8-Trifluoro-9-methoxy-10,11,12,13- tetrahydro-19H-5,18- methanodibenzo[c,f]pyrido[3,2-j] [1,5,9]triazacyclotridecine-15,19(14H)-dione, Formic acid salt | 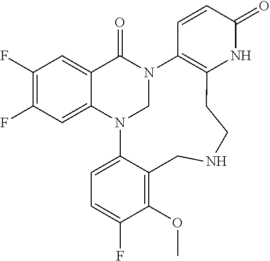 [SMILES: COc1c(F)ccc2c1CNCCc1[nH]c(=O)ccc1N1CN2c2cc(F)c(F)cc2C1=O] | 6.1 |
| | | 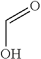 | |
| 71 | 2,8-Difluoro-9-methyl-15,19-dioxo- 10,11,12,13,14,15-hexahydro-19H-5,18- methanodibenzo[c,f]pyrido[3,2-j] [1,5,9]triazacyclotridecine-3-carbonitrile hydrochloride | 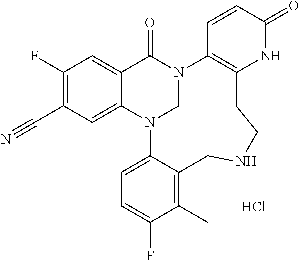 [SMILES: Cc1c(F)ccc2c1CNCCc1[nH]c(=O)ccc1N1CN2c2cc(C#N)c(F)cc2C1=O ; Cl] | 7.6 |
| 72 | 2-Chloro-8,9-difluoro-15,19-dioxo- 10,11,12,13,14,15-hexahydro-19H-5,18- methanodibenzo[c,f]pyrido[3,2- j][1,5,9]triazacyclotridecine-3-carbonitrile hydrochloride | 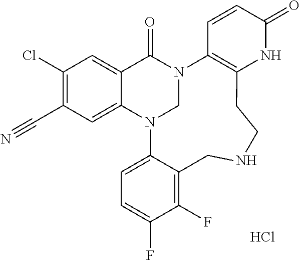 [SMILES: N#Cc1cc2c(cc1Cl)C(=O)N1CN2c2ccc(F)c(F)c2CNCCc2[nH]c(=O)ccc21 ; Cl] | 7.5 |
| 73 | 2,8-Difluoro-9-methoxy-15,19-dioxo- 10,11,12,13,14,15-hexahydro-19H-5,18- methanodibenzo[c,f]pyrido[3,2-j] [1,5,9]triazacyclotridecine-3-carbonitrile hydrochloride | 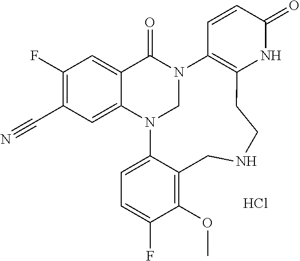 [SMILES: COc1c(F)ccc2c1CNCCc1[nH]c(=O)ccc1N1CN2c2cc(C#N)c(F)cc2C1=O ; Cl] | 6.1 |
| 74 | 8-Chloro-2,3,9-trifluoro-10,11,12,13- tetrahydro-19H-5,18- methanodibenzo[c,f]pyrido[3,2-j] [1,5,9]triazacyclotridecine-15,19(14H)-dione hydrochloride | 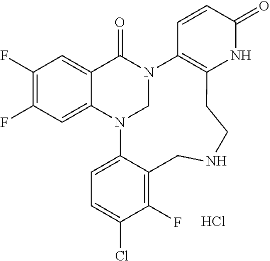 [SMILES: O=C1c2cc(F)c(F)cc2N2CN1c1ccc(=O)[nH]c1CCNCc1c2ccc(Cl)c1F ; Cl] | 7.3 |
| 75 | 9-(Difluoromethoxy)-2,3-difluoro- 10,11,12,13-tetrahydro-19H-5,18- methanodibenzo[c,f]pyrido[3,2-j] [1,5,9]triazacyclotridecine-15,19(14H)-dione hydrochloride | 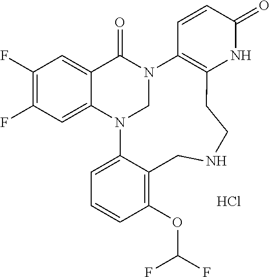 [SMILES: O=C1c2cc(F)c(F)cc2N2CN1c1ccc(=O)[nH]c1CCNCc1c(OC(F)F)cccc12 ; Cl] | 5.4 |
| 76 | 8-Chloro-2,3-difluoro-10,11,12,13-tetrahydro- 19H-5,18-methanodibenzo[c,f]pyrido[3,2-j] [1,5,9]triazacyclotridecine-15,19(14H)-dione hydrochloride | 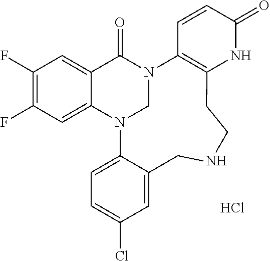 [SMILES: O=C1c2cc(F)c(F)cc2N2CN1c1ccc(=O)[nH]c1CCNCc1cc(Cl)ccc12 ; Cl] | 7.2 |
| 77 | 8,9-Dichloro-2,3-difluoro-10,11,12,13- tetrahydro-19H-5,18- methanodibenzo[c,f]pyrido[3,2-j] [1,5,9]triazacyclotridecine-15,19(14H)-dione hydrochloride | 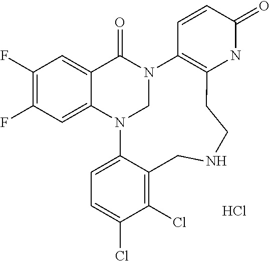 [SMILES: O=C1c2cc(F)c(F)cc2N2CN1c1ccc(=O)[nH]c1CCNCc1c2ccc(Cl)c1Cl ; Cl] | 8.7 |
| 78 | 2-(Trifluoromethyl)-10,11,12,13-tetrahydro- 19H-5,18-methanodibenzo[c,f]pyrido[3,2-j] [1,5,9]triazacyclotridecine-15,19(14H)-dione hydrochloride | 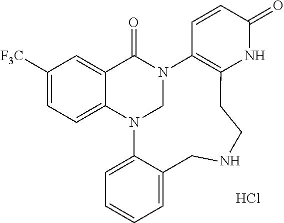 [SMILES: O=C1c2cc(C(F)(F)F)ccc2N2CN1c1ccc(=O)[nH]c1CCNCc1ccccc12 ; Cl] | 6.7 |
| 79 | 9-(Difluoromethyl)-2,3-difluoro-10,11,12,13- tetrahydro-19H-5,18- methanodibenzo[c,f]pyrido[3,2-j] [1,5,9]triazacyclotridecine-15,19(14H)-dione hydrochloride | 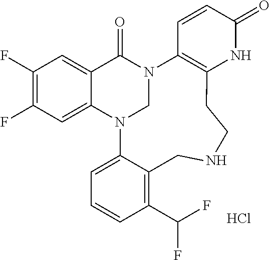 [SMILES: O=C1c2cc(F)c(F)cc2N2CN1c1ccc(=O)[nH]c1CCNCc1c(C(F)F)cccc12 ; Cl] | 7.1 |
| 80 | 2,9-Difluoro-3-(trifluoromethyl)-10,11,12,13- tetrahydro-19H-5,18- methanodibenzo[c,f]pyrido[3,2-j] [1,5,9]triazacyclotridecine-15,19(14H)-dione hydrochloride | 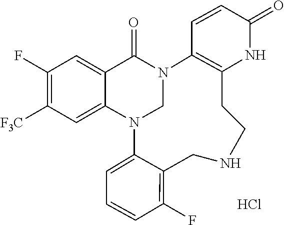 [SMILES: O=C1c2cc(F)c(C(F)(F)F)cc2N2CN1c1ccc(=O)[nH]c1CCNCc1c(F)cccc12 ; Cl] | 7.7 |
| 81 | 9-Chloro-2-fluoro-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 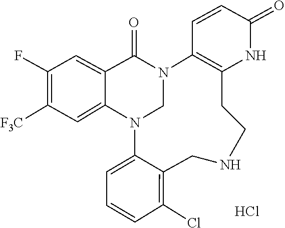 [SMILES: O=C1c2cc(F)c(C(F)(F)F)cc2N2CN1c1ccc(=O)[nH]c1CCNCc1c(Cl)cccc12 ; Cl] | 8.5 |
| 82 | 2,8-Difluoro-15,19-dioxo-3-(trifluoromethyl)- 10,11,12,13,14,15-hexahydro-19H-5,18- methanodibenzo[c,f]pyrido[3,2- j][1,5,9]triazacyclotridecine-9-carbonitrile hydrochloride | 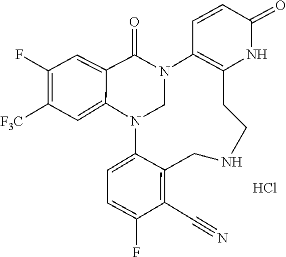 [SMILES: N#Cc1c(F)ccc2c1CNCCc1[nH]c(=O)ccc1N1CN2c2cc(C(F)(F)F)c(F)cc2C1=O ; Cl] | 7.9 |
| 83 | 2,3,8-Trifluoro-15,19-dioxo- 10,11,12,13,14,15-hexahydro-19H-5,18- methanodibenzo[c,f]pyrido[3,2-j] [1,5,9]triazacyclotridecine-9-carbonitrile | 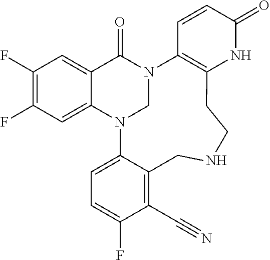 [SMILES: N#Cc1c(F)ccc2c1CNCCc1[nH]c(=O)ccc1N1CN2c2cc(F)c(F)cc2C1=O] | 6.8 |
| 84 | 2,3,8-Trifluoro-9-hydroxy-10,11,12,13- tetrahydro-19H-5,18- methanodibenzo[c,f]pyrido[3,2-j] [1,5,9]triazacyclotridecine-15,19(14H)-dione, formic acid salt | 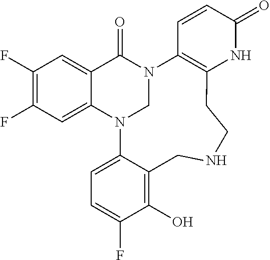 [SMILES: O=C1c2cc(F)c(F)cc2N2CN1c1ccc(=O)[nH]c1CCNCc1c2ccc(F)c1O] | 5.5 |
| | | 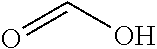 | |
| 85 | 2,8-Difluoro-9-hydroxy-3-(trifluoromethyl)- 10,11,12,13-tetrahydro-19H-5,18- methanodibenzo[c,f]pyrido[3,2-j] [1,5,9]triazacyclotridecine-15,19(14H)-dione hydrochloride | 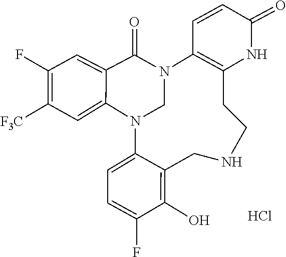 [SMILES: O=C1c2cc(F)c(C(F)(F)F)cc2N2CN1c1ccc(=O)[nH]c1CCNCc1c2ccc(F)c1O ; Cl] | 6.4 |
| 86 | 2,3-Difluoro-8-(trifluoromethoxy)- 10,11,12,13-tetrahydro-19H-5,18- methanodibenzo[c,f]pyrido[3,2-j] [1,5,9]triazacyclotridecine-15,19(14H)-dione hydrochloride | 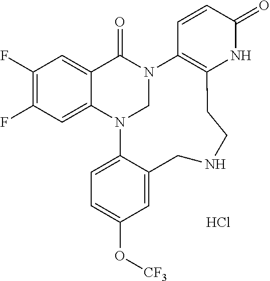 [SMILES: O=C1c2cc(F)c(F)cc2N2CN1c1ccc(=O)[nH]c1CCNCc1cc(OC(F)(F)F)ccc12 ; Cl] | 7.5 |
| 87 | 2,3,9-Trifluoro-8-(trifluoromethoxy)- 10,11,12,13-tetrahydro-19H-5,18- methanodibenzo[c,f]pyrido[3,2-j] [1,5,9]triazacyclotridecine-15,19(14H)-dione hydrochloride | 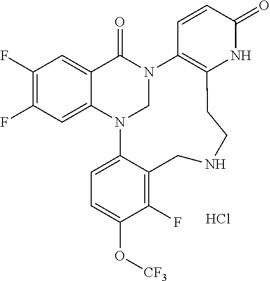 [SMILES: O=C1c2cc(F)c(F)cc2N2CN1c1ccc(=O)[nH]c1CCNCc1c2ccc(OC(F)(F)F)c1F ; Cl] | 7.9 |
| 88 | 2-Fluoro-15,19-dioxo-8-(trifluoromethoxy)- 10,11,12,13,14,15-hexahydro-19H-5,18- methanodibenzo[c,f]pyrido[3,2-j] [1,5,9]triazacyclotridecine-3-carbonitrile hydrochloride | 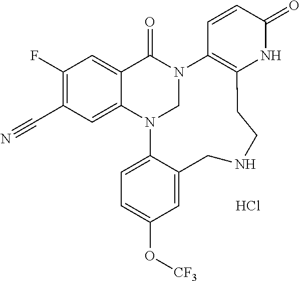 [SMILES: N#Cc1cc2c(cc1F)C(=O)N1CN2c2ccc(OC(F)(F)F)cc2CNCCc2[nH]c(=O)ccc21 ; Cl] | 7.7 |
| 89 | 2,9-Difluoro-15,19-dioxo-8- (trifluoromethoxy)-10,11,12,13,14,15- hexahydro-19H-5,18- methanodibenzo[c,f]pyrido[3,2-j] [1,5,9]triazacyclotridecine-3-carbonitrile hydrochloride | 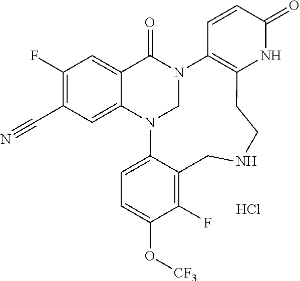 [SMILES: N#Cc1cc2c(cc1F)C(=O)N1CN2c2ccc(OC(F)(F)F)c(F)c2CNCCc2[nH]c(=O)ccc21 ; Cl] | 8.1 |
| 90 | 3,8-Difluoro-2-(trifluoromethyl)-10,11,12,13- tetrahydro-19H-5,18- methanodibenzo[f,i]pyrido[2,3- b][1,4,8]triazacyclotridecine-15,19(14H)-dione | 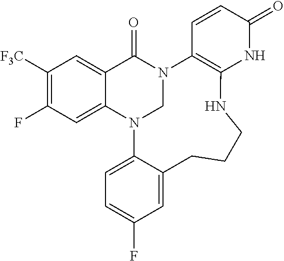 [SMILES: O=C1c2cc(C(F)(F)F)c(F)cc2N2CN1c1ccc(=O)[nH]c1NCCCc1cc(F)ccc12] | 7.7 |
| 91 | 2,3,8-Trifluoro-10,11,12,13-tetrahydro-19H- 5,18- methanodibenzo[f,i]pyrido[2,3b][1,4,8] triazacyclotridecine-15,19(14H)-dione | 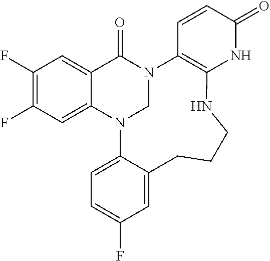 [SMILES: O=C1c2cc(F)c(F)cc2N2CN1c1ccc(=O)[nH]c1NCCCc1cc(F)ccc12] | 7.9 |
| 92 | 2,8,9-Trifluoro-3-(trifluoromethyl)- 10,11,12,13-tetrahydro-19H-5,18- methanodibenzo[f,i]pyrido[2,3- b][1,4,8]triazacyclotridecine-15,19(14H) | 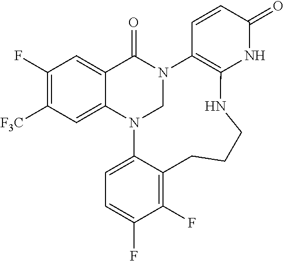 [SMILES: O=C1c2cc(F)c(C(F)(F)F)cc2N2CN1c1ccc(=O)[nH]c1NCCCc1c2ccc(F)c1F] | 8.4 |
| 93 | 3-Chloro-2,8-difluoro-10,11,12,13-tetrahydro- 19H-5,18- methanodibenzo[f,i]pyrido[2,3b][1,4,8]- triazacyclotridecine-15,19(14H) | 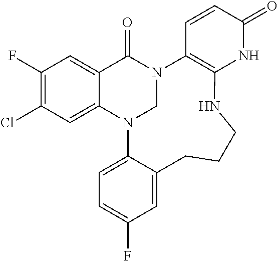 [SMILES: O=C1c2cc(F)c(Cl)cc2N2CN1c1ccc(=O)[nH]c1NCCCc1cc(F)ccc12] | 8.1 |
| 94 | 3-Chloro-2,8,9-trifluoro-10,11,12,13- tetrahydro-19H-5,18- methanodibenzo[f,i]pyrido[2,3- b][1,4,8]triazacyclotridecine-15,19(14H) | 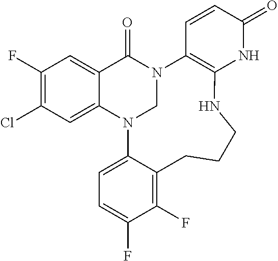 [SMILES: O=C1c2cc(F)c(Cl)cc2N2CN1c1ccc(=O)[nH]c1NCCCc1c2ccc(F)c1F] | 8.1 |
| 95 | 2,8-Difluoro-15,19-dioxo-10,11,12,13,14,15- hexahydro-19H-5,18- methanodibenzo[f,i]pyrido[2,3- b][1,4,8]triazacyclotridecine-3-carbonitrile | 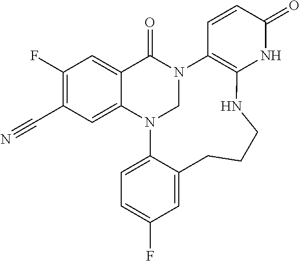 [SMILES: N#Cc1cc2c(cc1F)C(=O)N1CN2c2ccc(F)cc2CCCNc2[nH]c(=O)ccc21] | 7.5 |
| 96 | 2-Chloro-3,8-difluoro-10,11,12,13-tetrahydro- 19H-5,18-methanodibenzo[f,i]pyrido[2,3- b][1,4,8]triazacyclotridecine-15,19(14H)-dione | 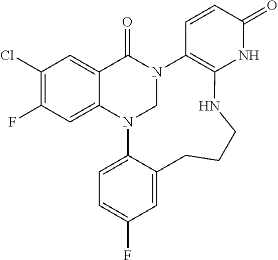 [SMILES: O=C1c2cc(Cl)c(F)cc2N2CN1c1ccc(=O)[nH]c1NCCCc1cc(F)ccc12] | 7.5 |
| 97 | 1,8,9-Trifluoro-2-(trifluoromethyl)- 10,11,12,13-tetrahydro-19H-5,18- methanodibenzo[f,i]pyrido[2,3- b][1,4,8]triazacyclotridecine-15,19(14H) | 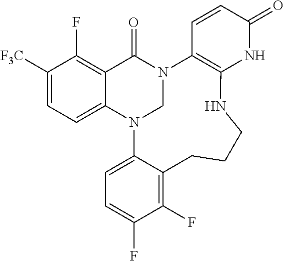 [SMILES: O=C1c2c(ccc(C(F)(F)F)c2F)N2CN1c1ccc(=O)[nH]c1NCCCc1c2ccc(F)c1F] | 7.6 |
| 98 | 2,3,8,9-Tetrafluoro-10,11,12,13-tetrahydro- 19H-5,18-methanodibenzo[f,i]pyrido[2,3- b][1,4,8]triazacyclotridecine-15,19(14H), formic acid salt | 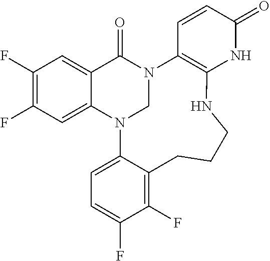 [SMILES: O=C1c2cc(F)c(F)cc2N2CN1c1ccc(=O)[nH]c1NCCCc1c2ccc(F)c1F] | 7.6 |
| | | 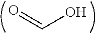 | |
| 99 | 2,8,9-Trifluoro-15,19-dioxo- 10,11,12,13,14,15-hexahydro-19H-5,18- methanodibenzo[f,i]pyrido[2,3- b][1,4,8]triazacyclotridecine-3-carbonitrile | 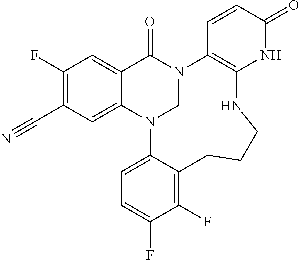 [SMILES: N#Cc1cc2c(cc1F)C(=O)N1CN2c2ccc(F)c(F)c2CCCNc2[nH]c(=O)ccc21] | 7.7 |
| 100 | 2-Chloro-8-fluoro-15,19-dioxo- 10,11,12,13,14,15-hexahydro-19H-5,18- methanodibenzo[f,i]pyrido[2,3- b][1,4,8]triazacyclotridecine-3-carbonitrile, hydrochloride | 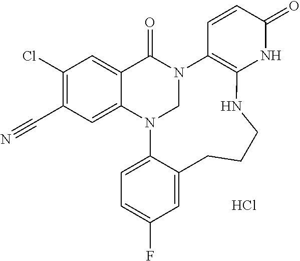 [SMILES: N#Cc1cc2c(cc1Cl)C(=O)N1CN2c2ccc(F)cc2CCCNc2[nH]c(=O)ccc21 ; Cl] | 7.5 |
| 101 | 2-Chloro-3,8,9-trifluoro-10,11,12,13- tetrahydro-19H-5,18- methanodibenzo[f,i]pyrido[2,3- b][1,4,8]triazacyclotridecine-15,19(14H)- dione, hydrochloride | 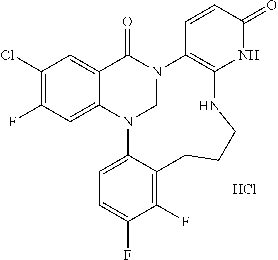 [SMILES: O=C1c2cc(Cl)c(F)cc2N2CN1c1ccc(=O)[nH]c1NCCCc1c2ccc(F)c1F ; Cl] | 7.8 |
| 102 | 2,8-Difluoro-3-(trifluoromethyl)-10,11,12,13- tetrahydro-19H-5,18- methanodibenzo[b,l]pyrido[3,2- f[1,5,9]triazacyclotridecine-15,19(14H)-dione | 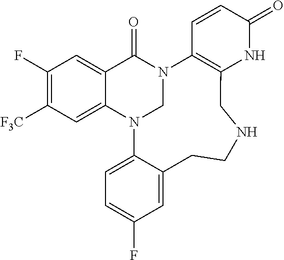 [SMILES: O=C1c2cc(F)c(C(F)(F)F)cc2N2CN1c1ccc(=O)[nH]c1CNCCc1cc(F)ccc12] | 7.1 |
| 103 | 8-Fluoro-2-(trifluoromethyl)-10,11,12,13- tetrahydro-19H-5,18- methanodibenzo[b,l]pyrido[3,2- f[1,5,9]triazacyclotridecine-15,19(14H)-dione | 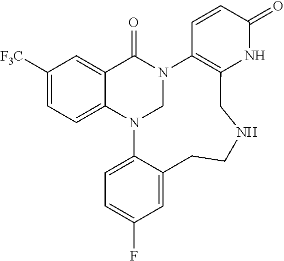 [SMILES: O=C1c2cc(C(F)(F)F)ccc2N2CN1c1ccc(=O)[nH]c1CNCCc1cc(F)ccc12] | 6.4 |
| 104 | 3⁴-Fluoro-2⁶-(trifluoromethyl)- 1¹,1²,2¹,2²,2³,2⁴-hexahydro-7-aza-2(3,1)- quinazolina-1(5,6)-pyridina-3(1,2)- benzenacyclooctaphane-1²,2⁴-dione | 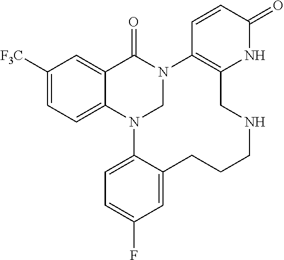 [SMILES: O=C1c2cc(C(F)(F)F)ccc2N2CN1c1ccc(=O)[nH]c1CNCCCc1cc(F)ccc12] | 6.9 |
| 105 | 2⁶,3⁴-Difluoro-2⁷-(trifluoromethyl)- 1¹,1²,2¹,2²,2³,2⁴-hexahydro-7-aza-2(3,1)- quinazolina-1(5,6)-pyridina-3(1,2)- benzenacyclooctaphane-1²,2⁴-dione hydrochloride | 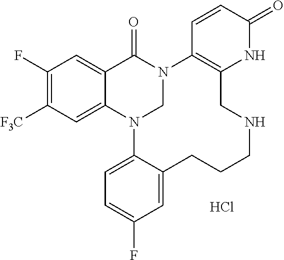 [SMILES: O=C1c2cc(F)c(C(F)(F)F)cc2N2CN1c1ccc(=O)[nH]c1CNCCCc1cc(F)ccc12 ; Cl] | 7.8 |
| 106 | 2⁶,3³,3⁴-Trifluoro-2⁷-(trifluoromethyl)- 1¹,1²,2¹,2²,2³,2⁴-hexahydro-7-aza-2(3,1)- quinazolina-1(5,6)-pyridina-3(1,2)- benzenacyclooctaphane-1²,2⁴-dione hydrochloride | 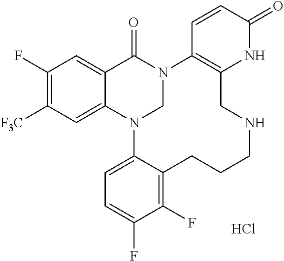 [SMILES: O=C1c2cc(F)c(C(F)(F)F)cc2N2CN1c1ccc(=O)[nH]c1CNCCCc1c2ccc(F)c1F ; Cl] | 8.1 |
| 107 | 2⁶,3⁴-Difluoro-2⁷-(trifluoromethyl)- 1¹,1²,2¹,2²,2³,2⁴-hexahydro-5-aza-2(3,1)- quinazolina-1(5,6)-pyridina-3(1,2)- benzenacyclooctaphane-1²,2⁴-dione hydrochloride | 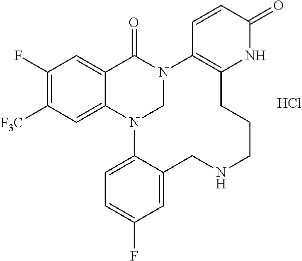 [SMILES: O=C1c2cc(F)c(C(F)(F)F)cc2N2CN1c1ccc(=O)[nH]c1CCCNCc1cc(F)ccc12 ; Cl] | 7.3 |
| 108 | 2⁶,3⁴-Difluoro-2⁷-(trifluoromethyl)- 1¹,1²,2¹,2²,2³,2⁴-hexahydro-5-aza-2(3,1)- quinazolina-1(5,6)-pyridina-3(1,2)- benzenacyclooctaphane-1²,2⁴-dione hydrochloride | 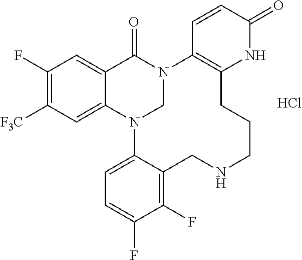 [SMILES: O=C1c2cc(F)c(C(F)(F)F)cc2N2CN1c1ccc(=O)[nH]c1CCCNCc1c2ccc(F)c1F ; Cl] | 7.6 |
| 109 | 3³-Chloro-2⁶,2⁷,3⁴-trifluoro-1¹, 1²,2¹,2²,2³,2⁴- hexahydro-5-aza-2(3,1)-quinazolina-1(5,6)- pyridina-3(1,2)-benzenacyclooctaphane-1²,2⁴- dione hydrochloride | 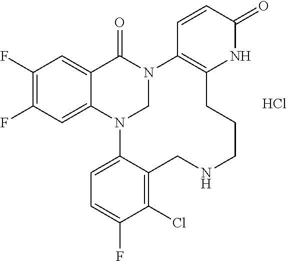 [SMILES: O=C1c2cc(F)c(F)cc2N2CN1c1ccc(=O)[nH]c1CCCNCc1c2ccc(F)c1Cl ; Cl] | 6.8 |
| 110 | 3³-Chloro-2⁶,3⁴-difluoro-1²,2⁴-dioxo- 1¹,1²,2¹,2²,2³,2⁴-hexahydro-5-aza-2(3,1)- quinazolina-1(5,6)-pyridina-3(1,2)- benzenacyclooctaphane-2⁷-carbonitrile hydrochloride | 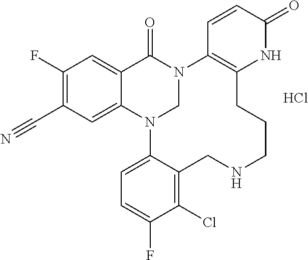 [SMILES: N#Cc1cc2c(cc1F)C(=O)N1CN2c2ccc(F)c(Cl)c2CNCCCc2[nH]c(=O)ccc21 ; Cl] | 6.8 |
| 111 | 2⁶,3⁴-Difluoro-2⁷-(trifluoromethyl)- 1¹,1²,2¹,2²,2³,2⁴-hexahydro-6-aza-2(3,1)- quinazolina-1(5,6)-pyridina-3(1,2)- benzenacyclooctaphane-1²,2⁴-dione hydrochloride | 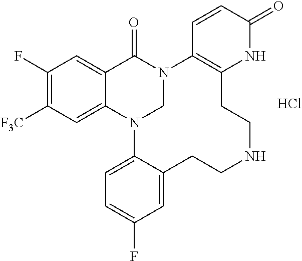 [SMILES: O=C1c2cc(F)c(C(F)(F)F)cc2N2CN1c1ccc(=O)[nH]c1CCNCCc1cc(F)ccc12 ; Cl] | 7.1 |
| 112 | 2⁶,3⁴-Difluoro-6-methyl-2⁷-(trifluoromethyl)- 1¹,1²,2¹,2²,2³,2⁴-hexahydro-6-aza-2(3,1)- quinazolina-1(5,6)-pyridina-3(1,2)- benzenacyclooctaphane-1²,2⁴-dione | 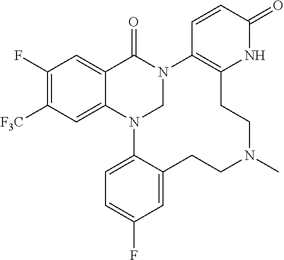 [SMILES: CN1CCc2cc(F)ccc2N2CN(C(=O)c3cc(F)c(C(F)(F)F)cc32)c2ccc(=O)[nH]c2CC1] | 6.2 |
| 113 | 3³-Chloro-2⁶,2⁷,3⁴-trifluoro-5-methyl- 1¹,1²,2¹,2²,2³,2⁴-hexahydro-5-aza-2(3,1)- quinazolina-1(5,6)-pyridina-3(1,2)- benzenacyclooctaphane-1²,2⁴-dione hydrochloride | 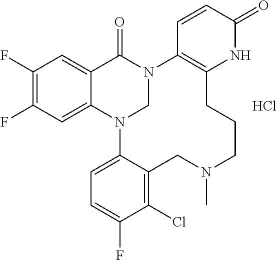 [SMILES: CN1CCCc2[nH]c(=O)ccc2N2CN(c3cc(F)c(F)cc3C2=O)c2ccc(F)c(Cl)c2C1 ; Cl] | 6.9 |
| 114 | 8-Fluoro-10-methyl-2-(trifluoromethyl)- 10,11,12,13-tetrahydro-19H-5,18- methanodibenzo[c,f]pyrido[3,2- j][1,5,9]triazacyclotridecine-15,19(14H)-dione hydrochloride | 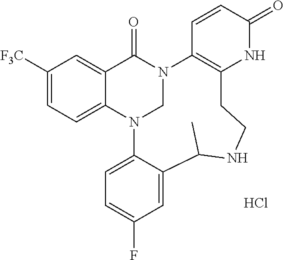 [SMILES: CC1NCCc2[nH]c(=O)ccc2N2CN(c3ccc(C(F)(F)F)cc3C2=O)c2ccc(F)cc21 ; Cl] | 6.8 |
| 115 | (10R)-8-Fluoro-10-methyl-2-(trifluoromethyl)- 10,11,12,13-tetrahydro-19H-5,18- methanodibenzo[c,f]pyrido[3,2- j][1,5,9]triazacyclotridecine-15,19(14H)-dione | 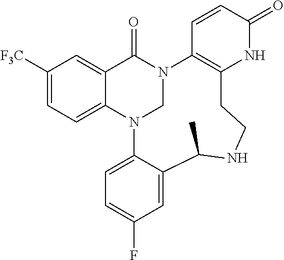 [SMILES: C[C@H]1NCCc2[nH]c(=O)ccc2N2CN(c3ccc(C(F)(F)F)cc3C2=O)c2ccc(F)cc21] | 7 |
| 116 | (10S)-8-Fluoro-10-methyl-2-(trifluoromethyl)- 10,11,12,13-tetrahydro-19H-5,18- methanodibenzo[c,f]pyrido[3,2- j][1,5,9]triazacyclotridecine-15,19(14H)-dione | 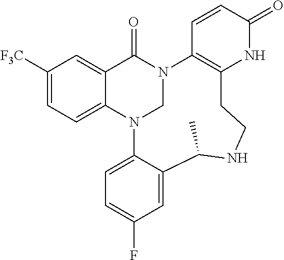 [SMILES: C[C@@H]1NCCc2[nH]c(=O)ccc2N2CN(c3ccc(C(F)(F)F)cc3C2=O)c2ccc(F)cc21] | 6.9 |
| 117 | 2,8-Difluoro-10-methyl-3-(trifluoromethyl)- 10,11,12,13-tetrahydro-19H-5,18- methanodibenzo[c,f]pyrido[3,2- j][1,5,9]triazacyclotridecine-15,19(14H)-dione hydrochloride | 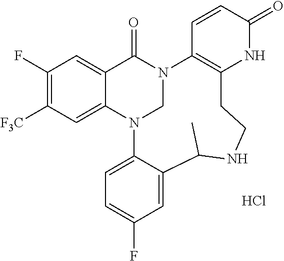 [SMILES: CC1NCCc2[nH]c(=O)ccc2N2CN(c3cc(C(F)(F)F)c(F)cc3C2=O)c2ccc(F)cc21 ; Cl] | 7.3 |
| 118 | (10R)-2,8-Difluoro-10-methyl-3- (trifluoromethyl)-10,11,12,13-tetrahydro-19H- 5,18-methanodibenzo[c,f]pyrido[3,2- j][1,5,9]triazacyclotridecine-15,19(14H)-dione | 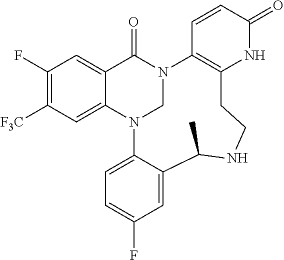 [SMILES: C[C@H]1NCCc2[nH]c(=O)ccc2N2CN(c3cc(C(F)(F)F)c(F)cc3C2=O)c2ccc(F)cc21] | 7.5 |
| 119 | (10S)-2,8-Difluoro-10-methyl-3- (trifluoromethyl)-10,11,12,13-tetrahydro-19H- 5,18-methanodibenzo[c,f]pyrido[3,2- j][1,5,9]triazacyclotridecine-15,19(14H)-dione | 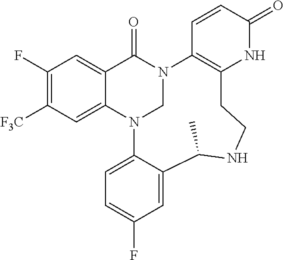 [SMILES: C[C@@H]1NCCc2[nH]c(=O)ccc2N2CN(c3cc(C(F)(F)F)c(F)cc3C2=O)c2ccc(F)cc21] | 6.2 |
| 120 | 3-Chloro-2,8-difluoro-10-methyl-10,11,12,13- tetrahydro-19H-5,18- methanodibenzo[c,f]pyrido[3,2- j][1,5,9]triazacyclotridecine-15,19(14H)-dione hydrochloride | 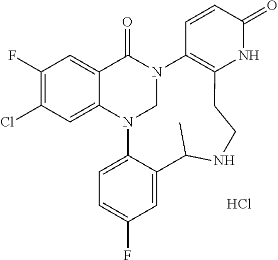 [SMILES: CC1NCCc2[nH]c(=O)ccc2N2CN(c3cc(Cl)c(F)cc3C2=O)c2ccc(F)cc21 ; Cl] | 7 |
| 121 | (10R)-3-Chloro-2,8-difluoro-10-methyl- 10,11,12,13-tetrahydro-19H-5,18- methanodibenzo[c,f]pyrido[3,2- j][1,5,9]triazacyclotridecine-15,19(14H)-dione | 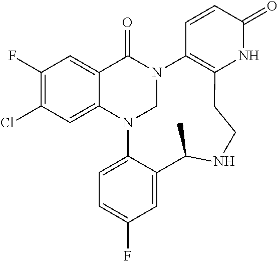 [SMILES: C[C@H]1NCCc2[nH]c(=O)ccc2N2CN(c3cc(Cl)c(F)cc3C2=O)c2ccc(F)cc21] | 7.4 |
| 122 | (10S)-3-Chloro-2,8-difluoro-10-methyl- 10,11,12,13-tetrahydro-19H-5,18- methanodibenzo[c,f]pyrido[3,2- j][1,5,9]triazacyclotridecine-15,19(14H)-dione | 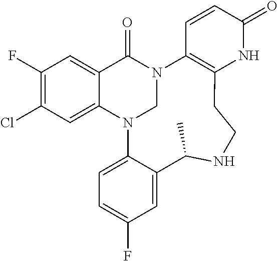 [SMILES: C[C@@H]1NCCc2[nH]c(=O)ccc2N2CN(c3cc(Cl)c(F)cc3C2=O)c2ccc(F)cc21] | 5.9 |
| 123 | 2,8-Difluoro-12-methyl-3-(trifluoromethyl)- 10,11,12,13-tetrahydro-19H-5,18- methanodibenzo[c,f]pyrido[3,2- j][1,5,9]triazacyclotridecine-15,19(14H)-dione hydrochloride | 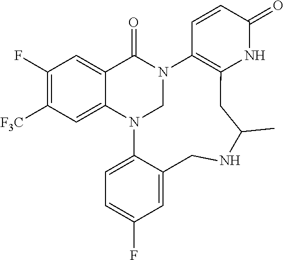 [SMILES: CC1Cc2[nH]c(=O)ccc2N2CN(c3ccc(F)cc3CN1)c1cc(C(F)(F)F)c(F)cc1C2=O] | 6.1 |
| 124 | 2,8-Difluoro-12-methyl-3-(trifluoromethyl)- 10,11,12,13-tetrahydro-19H-5,18- methanodibenzo[c,f]pyrido[3,2- j][1,5,9]triazacyclotridecine-15,19(14H)-dione, Isomer 1 | 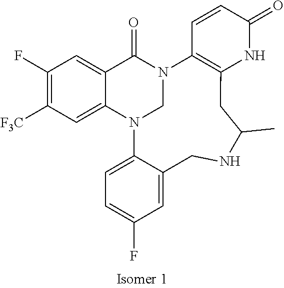 [SMILES: CC1Cc2[nH]c(=O)ccc2N2CN(c3ccc(F)cc3CN1)c1cc(C(F)(F)F)c(F)cc1C2=O] | 5.8 |
| 125 | 2,8-Difluoro-12-methyl-3-(trifluoromethyl)- 10,11,12,13-tetrahydro-19H-5,18- methanodibenzo[c,f]pyrido[3,2- j][1,5,9]triazacyclotridecine-15,19(14H)-dione, Isomer 2 | 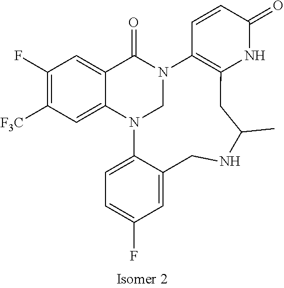 [SMILES: CC1Cc2[nH]c(=O)ccc2N2CN(c3ccc(F)cc3CN1)c1cc(C(F)(F)F)c(F)cc1C2=O] | 6.2 |
| 126 | 10-Ethyl-2,8-difluoro-3-(trifluoromethyl)- 10,11,12,13-tetrahydro-19H-5,18- methanodibenzo[c,f]pyrido[3,2- j][1,5,9]triazacyclotridecine-15,19(14H)-dione hydrochloride | 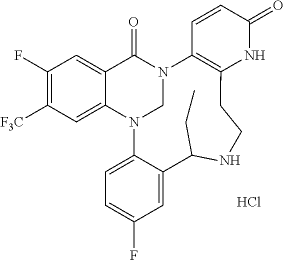 [SMILES: CCC1NCCc2[nH]c(=O)ccc2N2CN(c3cc(C(F)(F)F)c(F)cc3C2=O)c2ccc(F)cc21 ; Cl] | 7 |
| 127 | (10R)-10-Ethyl-2,8-difluoro-3- (trifluoromethyl)-10,11,12,13-tetrahydro-19H- 5,18-methanodibenzo[c,f]pyrido[3,2- j][1,5,9]triazacyclotridecine-15,19(14H)-dione | 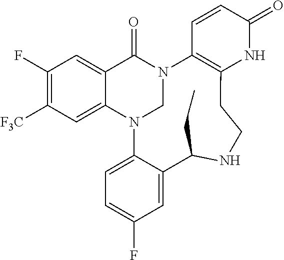 [SMILES: CC[C@H]1NCCc2[nH]c(=O)ccc2N2CN(c3cc(C(F)(F)F)c(F)cc3C2=O)c2ccc(F)cc21] | 7.2 |
| 128 | (10S)-10-Ethyl-2,8-difluoro-3- (trifluoromethyl)-10,11,12,13-tetrahydro-19H- 5,18-methanodibenzo[c,f]pyrido[3,2- j][1,5,9]triazacyclotridecine-15,19(14H)-dione | 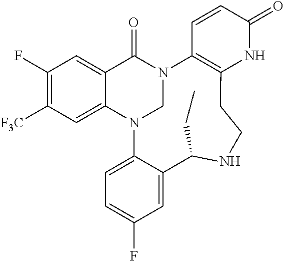 [SMILES: CC[C@@H]1NCCc2[nH]c(=O)ccc2N2CN(c3cc(C(F)(F)F)c(F)cc3C2=O)c2ccc(F)cc21] | 6.2 |
| 129 | 2⁶,3⁴-Difluoro-2⁷-(trifluoromethyl)- 1¹,1²,2¹,2²,2³,2⁴-hexahydro-5-oxa-8-aza- 2(3,1)-quinazolina-1(5,6)-pyridina-3(1,2)- benzenacyclooctaphane-1²,2⁴-dione | 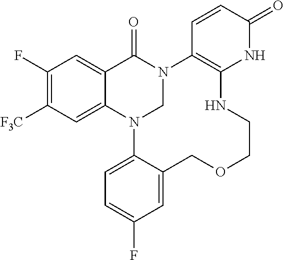 [SMILES: O=C1c2cc(F)c(C(F)(F)F)cc2N2CN1c1ccc(=O)[nH]c1NCCOCc1cc(F)ccc12] | 6.4 |
| 130 | 2,8,9-Trifluoro-12-methyl-3-(trifluoromethyl)- 10,11,12,13-tetrahydro-19H-5,18- methanodibenzo[f,i]pyrido[2,3- b][1,4,8]triazacyclotridecine-15,19(14H)-dione | 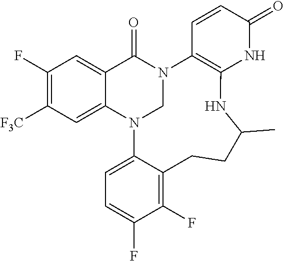 [SMILES: CC1CCc2c(ccc(F)c2F)N2CN(C(=O)c3cc(F)c(C(F)(F)F)cc32)c2ccc(=O)[nH]c2N1] | 6.8 |
| 131 | 2,8,9-Trifluoro-10-methyl-3-(trifluoromethyl)- 10,11,12,13-tetrahydro-19H-5,18- methanodibenzo[f,i]pyrido[2,3- b][1,4,8]triazacyclotridecine-15,19(14H)-dione | 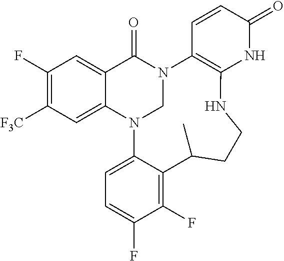 [SMILES: CC1CCNc2[nH]c(=O)ccc2N2CN(c3cc(C(F)(F)F)c(F)cc3C2=O)c2ccc(F)c(F)c21] | 7.8 |
| 132 | (10S)-2,8,9-Trifluoro-10-methyl-3- (trifluoromethyl)-10,11,12,13-tetrahydro-19H- 5,18-methanodibenzo[f,i]pyrido[2,3- b][1,4,8]triazacyclotridecine-15,19(14H)-dione | 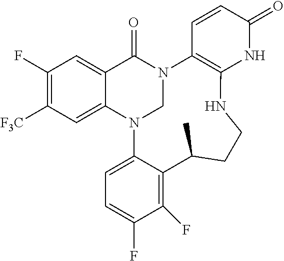 [SMILES: C[C@H]1CCNc2[nH]c(=O)ccc2N2CN(c3cc(C(F)(F)F)c(F)cc3C2=O)c2ccc(F)c(F)c21] | 8.2 |
| 133 | (10R)-2,8,9-Trifluoro-10-methyl-3- (trifluoromethyl)-10,11,12,13-tetrahydro-19H- 5,18-methanodibenzo[f,i]pyrido[2,3- b][1,4,8]triazacyclotridecine-15,19(14H)-dione | 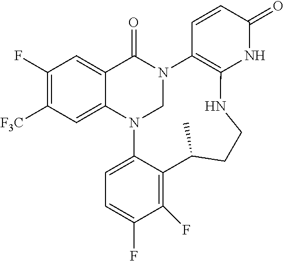 [SMILES: C[C@@H]1CCNc2[nH]c(=O)ccc2N2CN(c3cc(C(F)(F)F)c(F)cc3C2=O)c2ccc(F)c(F)c21] | 7.4 |
| 134 | 2,8-Difluoro-13,13-dimethyl-3- (trifluoromethyl)-10,11,12,13-tetrahydro-19H- 5,18-methanodibenzo[c,f]pyrido[3,2- j][1,5,9]triazacyclotridecine-15,19(14H)-dione | 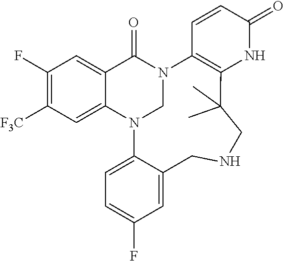 [SMILES: CC1(C)CNCc2cc(F)ccc2N2CN(C(=O)c3cc(F)c(C(F)(F)F)cc32)c2ccc(=O)[nH]c21] | 7.3 |
| 135 | 2⁶,3³,3⁴-Trifluoro-2⁷-(trifluoromethyl)- 1¹,1²,2¹,2²,2³,2⁴-hexahydro-5,8-diaza-2(3,1)- quinazolina-1(5,6)-pyridina-3(1,2)- benzenacyclooctaphane-1²,2⁴-dione hydrochloride | 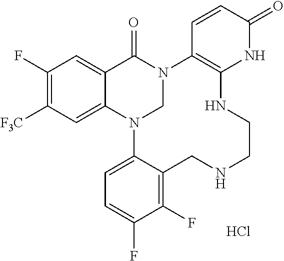 [SMILES: O=C1c2cc(F)c(C(F)(F)F)cc2N2CN1c1ccc(=O)[nH]c1NCCNCc1c2ccc(F)c1F ; Cl] | 7.4 |
| 136 | (4R)-2⁶,3⁴-Difluoro-4-methyl-2⁷- (trifluoromethyl)-1¹,1²,2¹,2²,2³,2⁴-hexahydro- 5-aza-2(3,1)-quinazolina-1(5,6)-pyridina- 3(1,2)-benzenacyclooctaphane-1²,2⁴-dione | 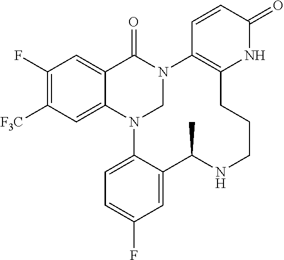 [SMILES: C[C@H]1NCCCc2[nH]c(=O)ccc2N2CN(c3cc(C(F)(F)F)c(F)cc3C2=O)c2ccc(F)cc21] | 6.9 |
| 137 | (4S)-2⁶,3⁴-Difluoro-4-methyl-2⁷- (trifluoromethyl)-1¹,1²,2¹,2²,2³,2⁴-hexahydro- 5-aza-2(3,1)-quinazolina-1(5,6)-pyridina- 3(1,2)-benzenacyclooctaphane-1²,2⁴-dione | 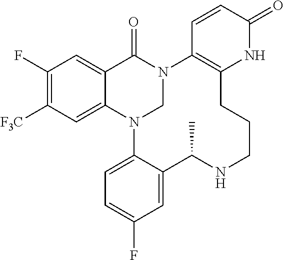 [SMILES: C[C@@H]1NCCCc2[nH]c(=O)ccc2N2CN(c3cc(C(F)(F)F)c(F)cc3C2=O)c2ccc(F)cc21] | 6.6 |

What is claimed is:

1. A compound of formula (I-a): 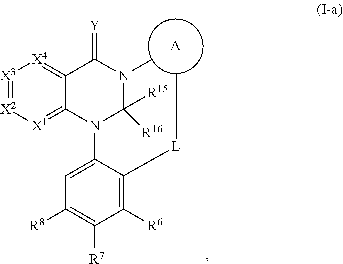
or a tautomer thereof, or a pharmaceutically acceptable salt thereof,
wherein:
Y is O or S;
X¹ is nitrogen or CR¹,
X² is nitrogen or CR²,
X³ is nitrogen or CR³, and
X⁴ is nitrogen or CR⁴,
provided no more than two of X¹, X², X³, and X⁴ are nitrogen;
ring A is: 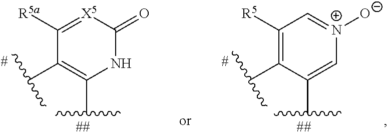
wherein [symbol] represents a covalent bond to the nitrogen atom of the bicyclic ring core of formula (I-a) and [symbol] represents a covalent bond to L of formula (I-a);
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
X⁵ is N or CR⁵;
each of R⁵ and R^{5a} is independently hydrogen, halo, or —(C₁-C₆)alkyl;
each of R⁶, R⁷ and R⁸ is independently hydrogen, halo, cyano, hydroxy, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-;
each of R¹⁵ and R¹⁶ is independently hydrogen or deuterium; and
L is (C₃-C₆)alkenylene, —NHCH₂CH₂NHCH₂—, —NHCH₂CH₂OCH₂—, a divalent linker of formula (L-ia), or a divalent linker of formula (L-iia): 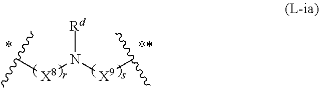
wherein:
each of X⁸ and X⁹ is independently —CR⁹R¹⁰—, wherein each of R⁹ and R¹⁰ is independently hydrogen or —(C₁-C₃)alkyl;
R^{d} is hydrogen or —(C₁-C₃)alkyl;
r is 1, 2, 3, or 4;
s is 1, 2, 3, or 4;
the sum of r and s is 2, 3, 4, or 5; and [symbol]
represents a covalent bond to ring A of formula (I-a) and [symbol] represents a covalent bond to the phenyl ring of formula (I-a); 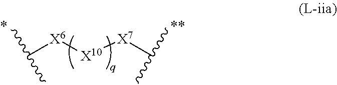
wherein:
X⁶ is —NR^{c}— or —CH₂—;
X⁷ is —CR¹¹R¹²—, —O— or —NH₂—, wherein each of R¹¹ and R¹² is independently hydrogen or —(C₁-C₃)alkyl, provided that when X⁷ is —NH₂—, X⁶ is —CH₂—;
each X¹⁰ is independently —CR¹³R¹⁴—, wherein each of R¹³ and R¹⁴ is independently hydrogen or —(C₁-C₃)alkyl;
R^{c} is hydrogen or —(C₁-C₃)alkyl;
q is 1, 2, 3, or 4;
and [symbol]
represents a covalent bond to ring A of formula (I-a) and [symbol] represents a covalent bond to the phenyl ring of formula (I-a).

2. The compound according to claim 1, which is a compound of formula (II): 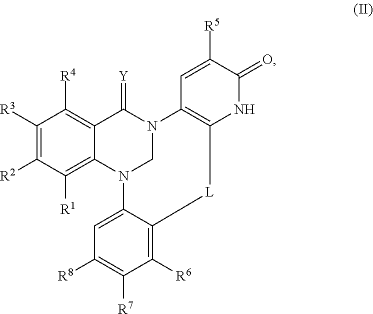
or a tautomer thereof, or a pharmaceutically acceptable salt thereof,
wherein:
Y is O or S;
each of R¹, R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
R⁵ is hydrogen, halo, or —(C₁-C₆)alkyl;
each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-; and
L is (C₃-C₆)alkenylene, a divalent linker of formula (L-i), or a divalent linker of formula (L-ii): 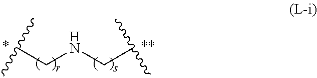
wherein:
r is 1, 2, 3, or 4;
s is 1, 2, 3, or 4;
the sum of r and s is 2, 3, 4, or 5; and [symbol]
represents a covalent bond to the pyridone ring of formula (II) and [symbol] represents a covalent bond to the phenyl ring of formula (II); 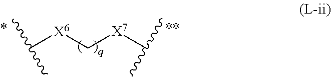
wherein:
X⁶ is —NR^{c}— or —CH₂—;
X⁷ is —CH₂—, —O— or —NH₂—, provided that when X⁷ is —NH₂—, X⁶ is —CH₂—;
R^{c} is hydrogen or —(C₁-C₃)alkyl;
q is 1, 2, 3, or 4; and [symbol]
represents a covalent bond to the pyridone ring of formula (II) and [symbol] represents a covalent bond to the phenyl ring of formula (II).

3. The compound according to claim 1, which is a compound of formula (III): 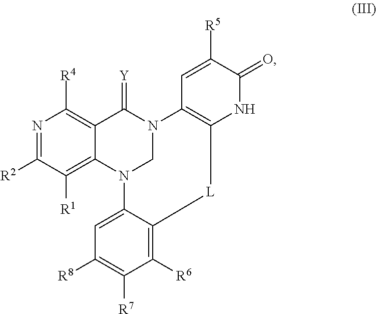
or a tautomer thereof, or a pharmaceutically acceptable salt thereof,
wherein:
Y is O or S;
each of R¹, R² and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
R⁵ is hydrogen, halo, or —(C₁-C₆)alkyl;
each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-; and
L is (C₃-C₆)alkenylene, a divalent linker of formula (L-i), or a divalent linker of formula (L-ii): 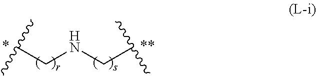
wherein:
r is 1, 2, 3, or 4;
s is 1, 2, 3, or 4;
the sum of r and s is 2, 3, 4, or 5; and [symbol]
represents a covalent bond to the pyridone ring of formula (III) and [symbol] represents a covalent bond to the phenyl ring of formula (III); 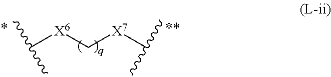
wherein:
X⁶ is —NR^{c}— or —CH₂—;
X⁷ is —CH₂—, —O— or —NH₂—, provided that when X⁷ is —NH₂—, X⁶ is —CH₂—;
R^{c} is hydrogen or —(C₁-C₃)alkyl;
q is 1, 2, 3, or 4; and [symbol]
represents a covalent bond to the pyridone ring of formula (III) and [symbol] represents a covalent bond to the phenyl ring of formula (III).

4. The compound according to claim 1, which is a compound of formula (IV): 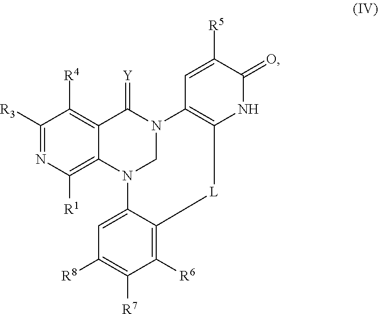
or a tautomer thereof, or a
pharmaceutically acceptable salt thereof,
wherein:
Y is O or S;
each of R¹, R³ and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
R⁵ is hydrogen, halo, or —(C₁-C₆)alkyl;
each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-; and
L is (C₃-C₆)alkenylene, a divalent linker of formula (L-i), or a divalent linker of formula (L-ii): 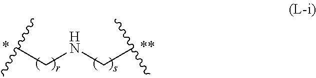
wherein:
r is 1, 2, 3, or 4;
s is 1, 2, 3, or 4;
the sum of r and s is 2, 3, 4, or 5; and [symbol]
represents a covalent bond to the pyridone ring of formula (IV) and [symbol] represents a covalent bond to the phenyl ring of formula (IV), 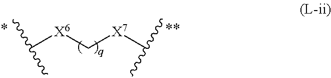
wherein:
X⁶ is —NR^{c}— or —CH₂—;
X⁷ is —CH₂—, —O— or —NH₂—, provided that when X⁷ is —NH₂—, X⁶ is —CH₂—;
R^{c} is hydrogen or —(C₁-C₃)alkyl;
q is 1, 2, 3, or 4; and [symbol]
represents a covalent bond to the pyridone ring of formula (IV) and [symbol] represents a covalent bond to the phenyl ring of formula (IV).

5. The compound according to claim 1, which is a compound of formula (V): 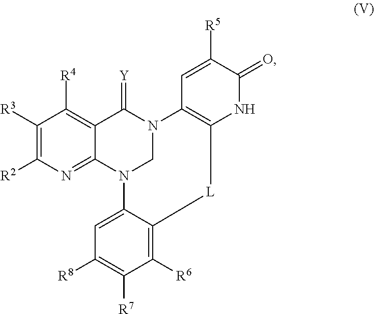
or a tautomer thereof, or a pharmaceutically acceptable salt thereof,
wherein:
Y is O or S;
each of R², R³, and R⁴ is independently hydrogen, halo, cyano, —NR^{a}R^{b}, —(C₁-C₆)alkyl, —(C₁-C₆)alkoxy, halo(C₁-C₆)alkyl-, or halo(C₁-C₆)alkoxy-;
R⁵ is hydrogen, halo, or —(C₁-C₆)alkyl;
each of R⁶, R⁷ and R⁸ is independently hydrogen, halo, —(C₁-C₆)alkyl, —(C₁-C₆)alkoxy, halo(C₁-C₆)alkyl-, or halo(C₁-C₆)alkoxy-;
each of R^{a} and R^{b} is independently hydrogen, —(C₁-C₆)alkyl, or halo(C₁-C₆)alkyl-; and
L is (C₃-C₆)alkenylene, a divalent linker of formula (L-i), or a divalent linker of formula (L-ii): 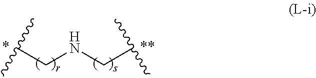
wherein:
r is 1, 2, 3, or 4;
s is 1, 2, 3, or 4;
the sum of r and s is 2, 3, 4, or 5; and [symbol]
represent a covalent bond to the pyridone ring of formula (V) and [symbol] represents a covalent bond to the phenyl ring of formula (V); 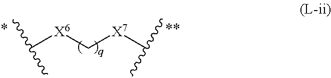
wherein:
X⁶ is —NR^{c}— or —CH₂—;
X⁷ is —CH₂—, —O— or —NH₂—, provided that when X⁷ is —NH₂—, X⁶ is —CH₂—;
R^{c} is hydrogen or —(C₁-C₃)alkyl;
q is 1, 2, 3, or 4; and [symbol]
represents a covalent bond to the pyridone ring of formula (V) and [symbol] represents a covalent bond to the phenyl ring of formula (V).

6. The compound, or tautomer thereof or pharmaceutically acceptable salt thereof according to claim 1, wherein Y is O.

7. The compound, or tautomer thereof or pharmaceutically acceptable salt thereof according to claim 1, wherein L is (C₃-C₆)alkenylene selected from the group consisting of: *—CH═CH—CH₂—**, *—CH₂—CH═CH—CH₂—**, *—CH₂CH₂—CH═CH—CH₂—**, and *—CH₂—CH═CH—CH₂CH₂CH₂—**, wherein “*” represents a covalent bond to the ring A or pyridone ring of formulas (Ia)-(V) and “**” represents a covalent bond to the phenyl ring of formulas (Ia)-(V).

8. The compound, or tautomer thereof or pharmaceutically acceptable salt thereof according to claim 1, wherein L is a divalent linker of formula (L-i): 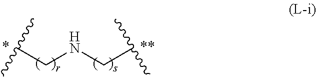
wherein:
r is 1, 2, 3, or 4;
s is 1, 2, 3, or 4;
the sum of r and s is 2, 3, 4, or 5; and [symbol]
represents a covalent bond to the ring A or pyridone ring of formulas (Ia)-(V) and [symbol] represents a covalent bond to the phenyl ring of formulas (Ia)-(V).

9. The compound, or tautomer thereof or pharmaceutically acceptable salt thereof according to claim 8, wherein L is a divalent linker of formula (L-i) selected from the group consisting of: 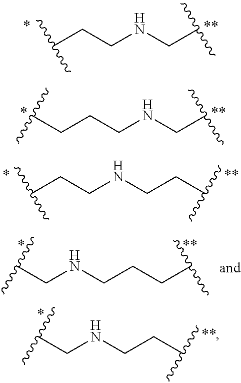
wherein [symbol] represents a covalent bond to the ring A or pyridone group of formulas (Ia)-(V) and [symbol] represents a covalent bond to the phenyl ring of formulas (Ia)-(V).

10. The compound, or tautomer thereof or pharmaceutically acceptable salt thereof according to claim 1, wherein L is a divalent linker of formula (L-ii): 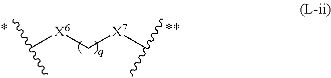
wherein:
X⁶ is —NR^{c}— or —CH₂—;
X⁷ is —CH₂—, —O— or —NH₂—, provided that when X⁷ is —NH₂—, X⁶ is —CH₂—;
R^{c} is hydrogen or —(C₁-C₃)alkyl;
q is 1, 2, 3, or 4; and [symbol]
represents a covalent bond to the ring A or pyridone ring of formulas (Ia)-(V) and [symbol] represents a covalent bond to the phenyl ring of formulas (Ia)-(V); wherein L is a divalent linker of formula (T-ii) selected from the group consisting of

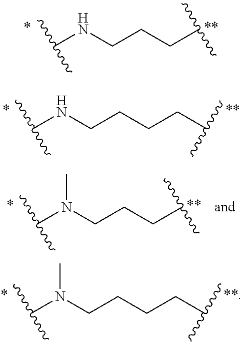

11. The compound, or tautomer thereof or pharmaceutically acceptable salt thereof according to claim 10, wherein X⁶ is —NR^{c}— and X⁷ is CH₂.

12. The compound, or tautomer thereof or pharmaceutically acceptable salt thereof according to claim 10, wherein L is a divalent linker of formula (L-ii) selected from the group consisting of: 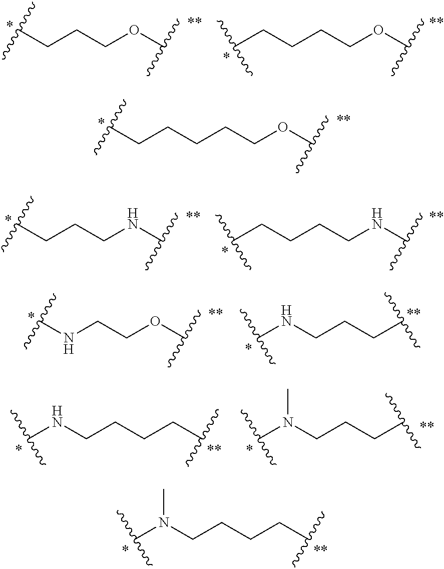
—CH₂CH₂CH₂CH₂—, —CH₂CH₂CH₂CH₂CH₂— and —CH₂CH₂CH₂CH₂CH₂CH₂—, wherein [symbol] represents a covalent bond to the ring A or pyridone ring of formulas (Ia)-(V) and [symbol] represents a covalent bond to the phenyl ring of formulas (Ia)-(V).

13. The compound, or tautomer thereof or pharmaceutically acceptable salt thereof according to claim 1, wherein L is a divalent linker of formula (L-ii) selected from the group consisting of: 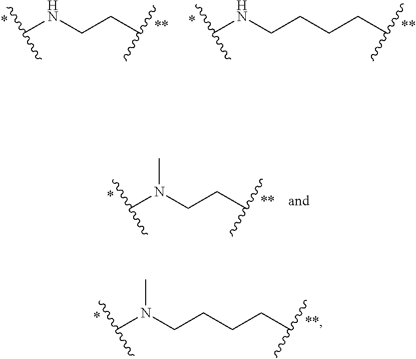
wherein [symbol] represents a covalent bond to the ring A or pyridone ring of formulas (Ia)-(V) and [symbol] represents a covalent bond to the phenyl ring of formulas (Ia)-(V).

14. The compound, or tautomer thereof or pharmaceutically acceptable salt thereof according to claim 1, wherein L is a divalent linker selected from the group consisting of 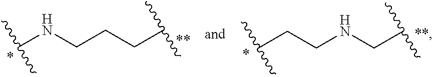
wherein [symbol] represents a covalent bond to the ring A or pyridone ring of formulas (Ia)-(V) and [symbol] represents a covalent bond to the phenyl ring of formulas (Ia)-(V).

15. A compound selected from the group consisting of: 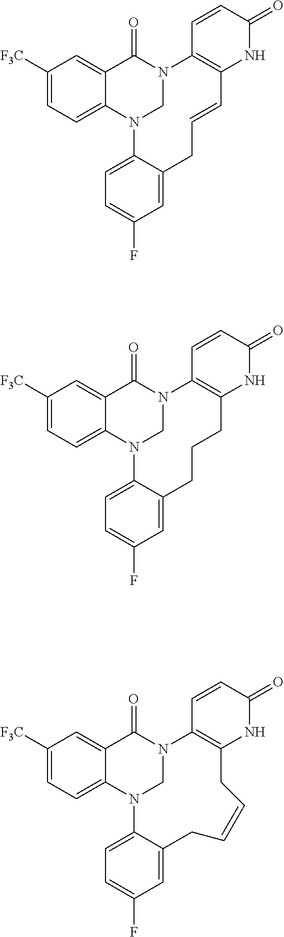 [SMILES: O=C1c2cc(C(F)(F)F)ccc2N2CN1c1ccc(=O)[nH]c1/C=C/Cc1cc(F)ccc12 ; O=C1c2cc(C(F)(F)F)ccc2N2CN1c1ccc(=O)[nH]c1CCCc1cc(F)ccc12 ; O=C1c2cc(C(F)(F)F)ccc2N2CN1c1ccc(=O)[nH]c1C/C=C\Cc1cc(F)ccc12] 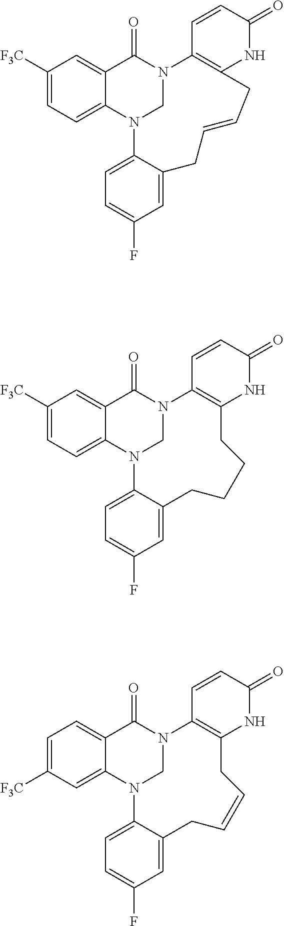 [SMILES: O=C1c2cc(C(F)(F)F)ccc2N2CN1c1ccc(=O)[nH]c1C/C=C/Cc1cc(F)ccc12 ; O=C1c2cc(C(F)(F)F)ccc2N2CN1c1ccc(=O)[nH]c1CCCCc1cc(F)ccc12 ; O=C1c2ccc(C(F)(F)F)cc2N2CN1c1ccc(=O)[nH]c1C/C=C\Cc1cc(F)ccc12] 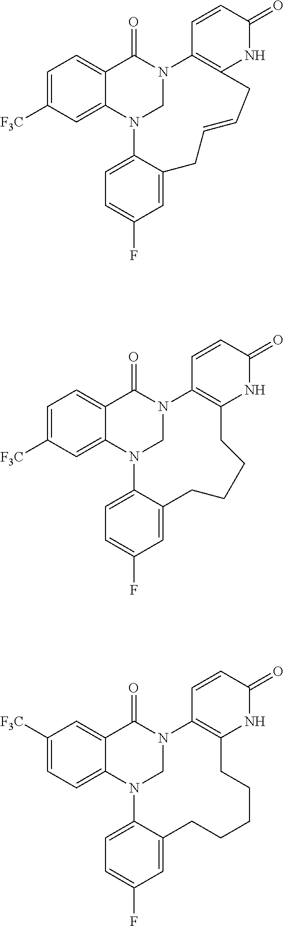 [SMILES: O=C1c2ccc(C(F)(F)F)cc2N2CN1c1ccc(=O)[nH]c1C/C=C/Cc1cc(F)ccc12 ; O=C1c2ccc(C(F)(F)F)cc2N2CN1c1ccc(=O)[nH]c1CCCCc1cc(F)ccc12 ; O=C1c2cc(C(F)(F)F)ccc2N2CN1c1ccc(=O)[nH]c1CCCCCc1cc(F)ccc12] 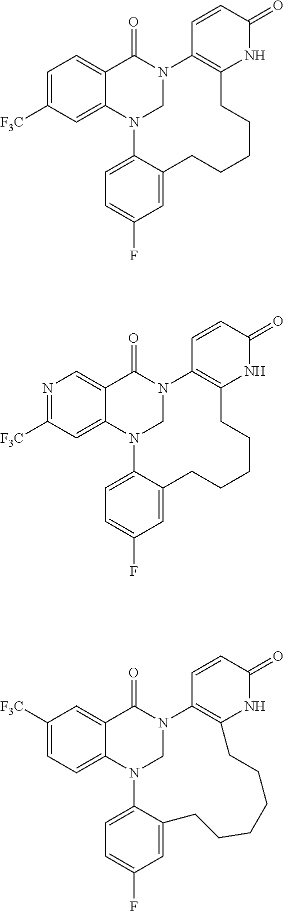 [SMILES: O=C1c2ccc(C(F)(F)F)cc2N2CN1c1ccc(=O)[nH]c1CCCCCc1cc(F)ccc12 ; O=C1c2cnc(C(F)(F)F)cc2N2CN1c1ccc(=O)[nH]c1CCCCCc1cc(F)ccc12 ; O=C1c2cc(C(F)(F)F)ccc2N2CN1c1ccc(=O)[nH]c1CCCCCCc1cc(F)ccc12] 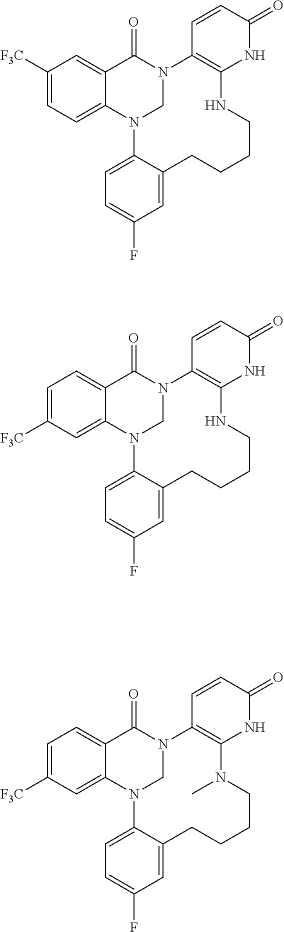 [SMILES: O=C1c2cc(C(F)(F)F)ccc2N2CN1c1ccc(=O)[nH]c1NCCCCc1cc(F)ccc12 ; O=C1c2ccc(C(F)(F)F)cc2N2CN1c1ccc(=O)[nH]c1NCCCCc1cc(F)ccc12 ; CN1CCCCc2cc(F)ccc2N2CN(C(=O)c3ccc(C(F)(F)F)cc32)c2ccc(=O)[nH]c21] 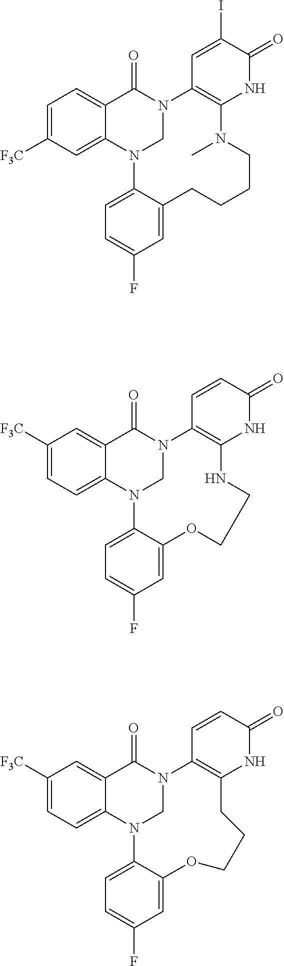 [SMILES: CN1CCCCc2cc(F)ccc2N2CN(C(=O)c3ccc(C(F)(F)F)cc32)c2cc(I)c(=O)[nH]c21 ; O=C1c2cc(C(F)(F)F)ccc2N2CN1c1ccc(=O)[nH]c1NCCOc1cc(F)ccc12 ; O=C1c2cc(C(F)(F)F)ccc2N2CN1c1ccc(=O)[nH]c1CCCOc1cc(F)ccc12] 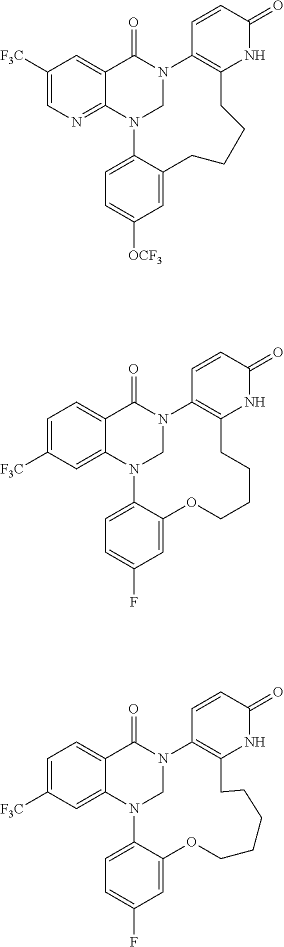 [SMILES: O=C1c2cc(C(F)(F)F)cnc2N2CN1c1ccc(=O)[nH]c1CCCCc1cc(OC(F)(F)F)ccc12 ; O=C1c2ccc(C(F)(F)F)cc2N2CN1c1ccc(=O)[nH]c1CCCCOc1cc(F)ccc12 ; O=C1c2ccc(C(F)(F)F)cc2N2CN1c1ccc(=O)[nH]c1CCCCCOc1cc(F)ccc12] 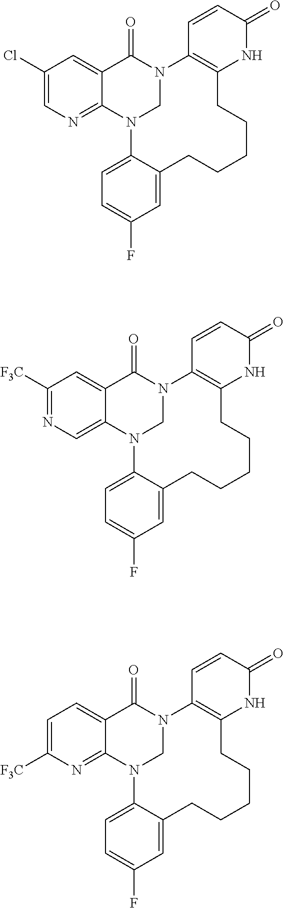 [SMILES: O=C1c2cc(Cl)cnc2N2CN1c1ccc(=O)[nH]c1CCCCCc1cc(F)ccc12 ; O=C1c2cc(C(F)(F)F)ncc2N2CN1c1ccc(=O)[nH]c1CCCCCc1cc(F)ccc12 ; O=C1c2ccc(C(F)(F)F)nc2N2CN1c1ccc(=O)[nH]c1CCCCCc1cc(F)ccc12] 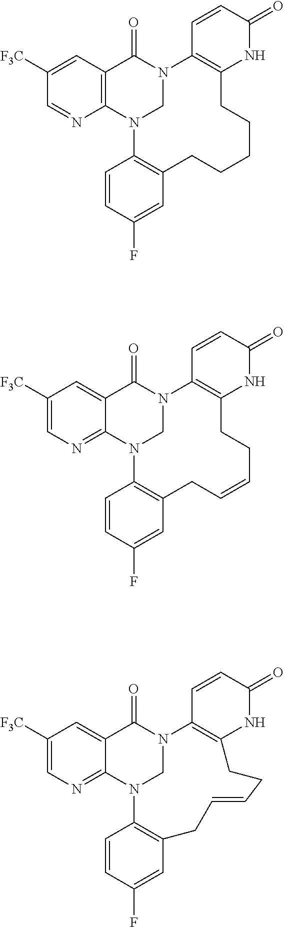 [SMILES: O=C1c2cc(C(F)(F)F)cnc2N2CN1c1ccc(=O)[nH]c1CCCCCc1cc(F)ccc12 ; O=C1c2cc(C(F)(F)F)cnc2N2CN1c1ccc(=O)[nH]c1CC/C=C\Cc1cc(F)ccc12 ; O=C1c2cc(C(F)(F)F)cnc2N2CN1c1ccc(=O)[nH]c1CC/C=C/Cc1cc(F)ccc12] 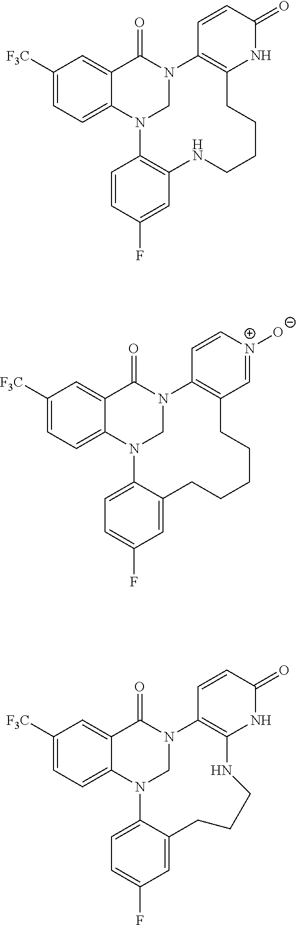 [SMILES: O=C1c2cc(C(F)(F)F)ccc2N2CN1c1ccc(=O)[nH]c1CCCCNc1cc(F)ccc12 ; O=C1c2cc(C(F)(F)F)ccc2N2CN1c1cc[n+]([O-])cc1CCCCCc1cc(F)ccc12 ; O=C1c2cc(C(F)(F)F)ccc2N2CN1c1ccc(=O)[nH]c1NCCCc1cc(F)ccc12] 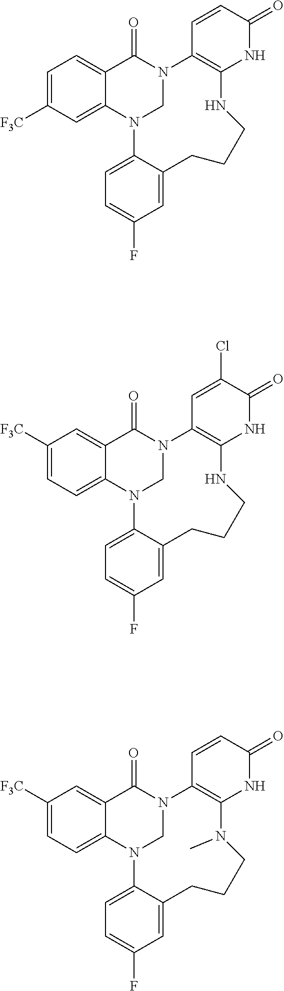 [SMILES: O=C1c2ccc(C(F)(F)F)cc2N2CN1c1ccc(=O)[nH]c1NCCCc1cc(F)ccc12 ; O=C1c2cc(C(F)(F)F)ccc2N2CN1c1cc(Cl)c(=O)[nH]c1NCCCc1cc(F)ccc12 ; CN1CCCc2cc(F)ccc2N2CN(C(=O)c3cc(C(F)(F)F)ccc32)c2ccc(=O)[nH]c21] 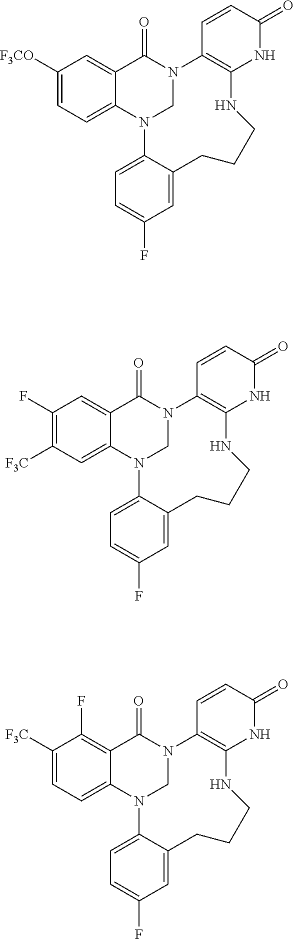 [SMILES: O=C1c2cc(OC(F)(F)F)ccc2N2CN1c1ccc(=O)[nH]c1NCCCc1cc(F)ccc12 ; O=C1c2cc(F)c(C(F)(F)F)cc2N2CN1c1ccc(=O)[nH]c1NCCCc1cc(F)ccc12 ; O=C1c2c(ccc(C(F)(F)F)c2F)N2CN1c1ccc(=O)[nH]c1NCCCc1cc(F)ccc12] 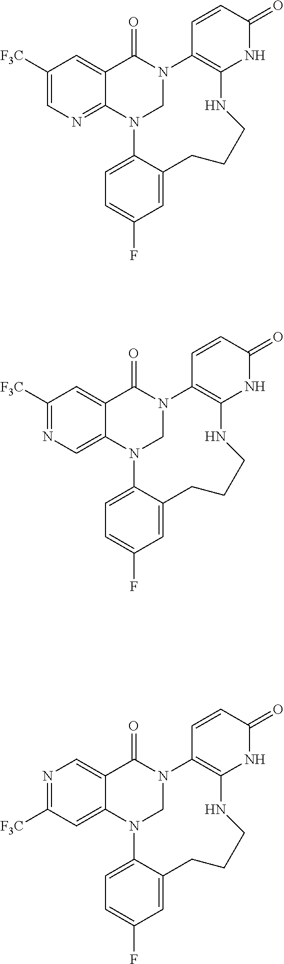 [SMILES: O=C1c2cc(C(F)(F)F)cnc2N2CN1c1ccc(=O)[nH]c1NCCCc1cc(F)ccc12 ; O=C1c2cc(C(F)(F)F)ncc2N2CN1c1ccc(=O)[nH]c1NCCCc1cc(F)ccc12 ; O=C1c2cnc(C(F)(F)F)cc2N2CN1c1ccc(=O)[nH]c1NCCCc1cc(F)ccc12] 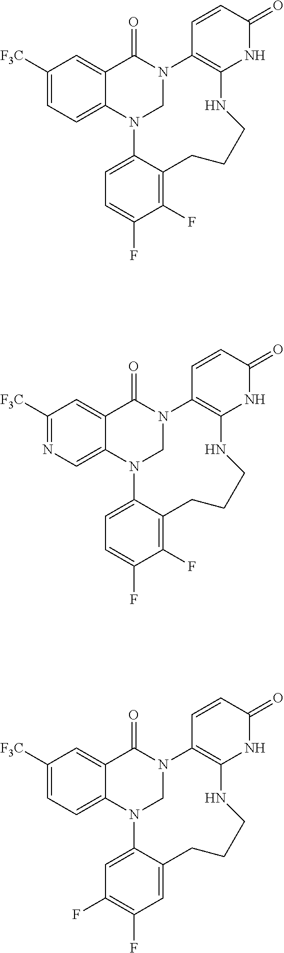 [SMILES: O=C1c2cc(C(F)(F)F)ccc2N2CN1c1ccc(=O)[nH]c1NCCCc1c2ccc(F)c1F ; O=C1c2cc(C(F)(F)F)ncc2N2CN1c1ccc(=O)[nH]c1NCCCc1c2ccc(F)c1F ; O=C1c2cc(C(F)(F)F)ccc2N2CN1c1ccc(=O)[nH]c1NCCCc1cc(F)c(F)cc12] 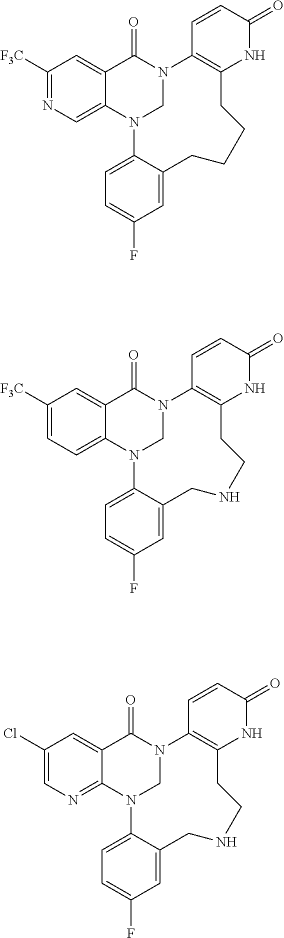 [SMILES: O=C1c2cc(C(F)(F)F)ncc2N2CN1c1ccc(=O)[nH]c1CCCCc1cc(F)ccc12 ; O=C1c2cc(C(F)(F)F)ccc2N2CN1c1ccc(=O)[nH]c1CCNCc1cc(F)ccc12 ; O=C1c2cc(Cl)cnc2N2CN1c1ccc(=O)[nH]c1CCNCc1cc(F)ccc12] 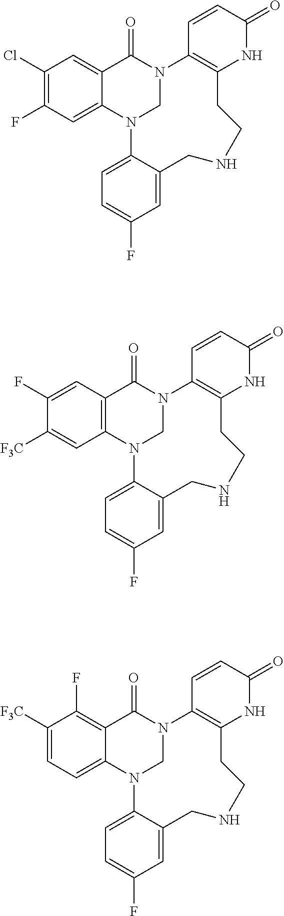 [SMILES: O=C1c2cc(Cl)c(F)cc2N2CN1c1ccc(=O)[nH]c1CCNCc1cc(F)ccc12 ; O=C1c2cc(F)c(C(F)(F)F)cc2N2CN1c1ccc(=O)[nH]c1CCNCc1cc(F)ccc12 ; O=C1c2c(ccc(C(F)(F)F)c2F)N2CN1c1ccc(=O)[nH]c1CCNCc1cc(F)ccc12] 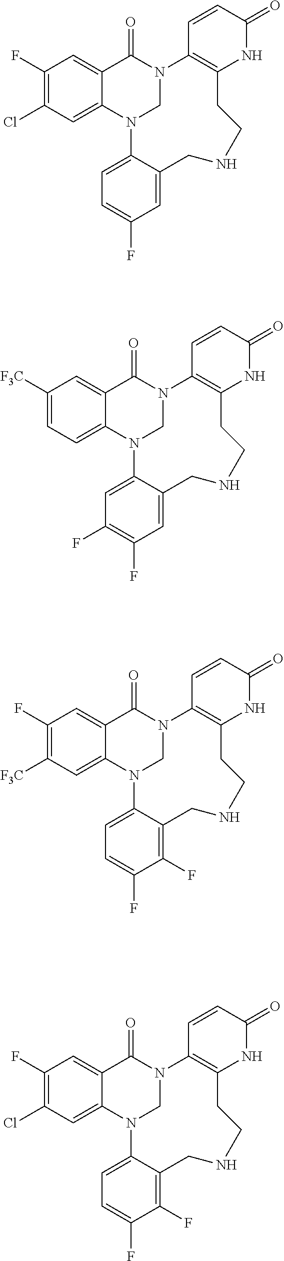 [SMILES: O=C1c2cc(F)c(Cl)cc2N2CN1c1ccc(=O)[nH]c1CCNCc1cc(F)ccc12 ; O=C1c2cc(C(F)(F)F)ccc2N2CN1c1ccc(=O)[nH]c1CCNCc1cc(F)c(F)cc12 ; O=C1c2cc(F)c(C(F)(F)F)cc2N2CN1c1ccc(=O)[nH]c1CCNCc1c2ccc(F)c1F ; O=C1c2cc(F)c(Cl)cc2N2CN1c1ccc(=O)[nH]c1CCNCc1c2ccc(F)c1F] 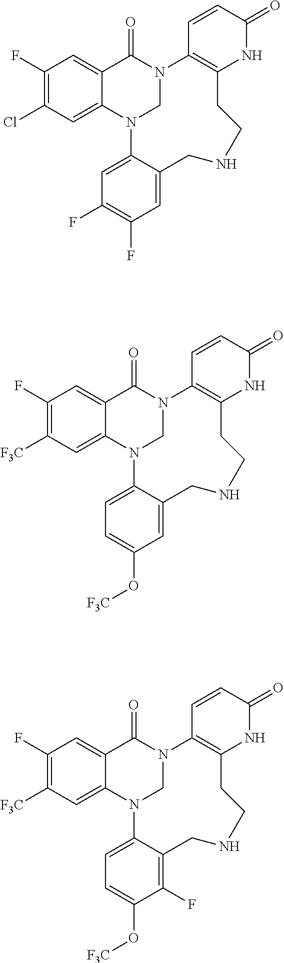 [SMILES: O=C1c2cc(F)c(Cl)cc2N2CN1c1ccc(=O)[nH]c1CCNCc1cc(F)c(F)cc12 ; O=C1c2cc(F)c(C(F)(F)F)cc2N2CN1c1ccc(=O)[nH]c1CCNCc1cc(OC(F)(F)F)ccc12 ; O=C1c2cc(F)c(C(F)(F)F)cc2N2CN1c1ccc(=O)[nH]c1CCNCc1c2ccc(OC(F)(F)F)c1F] 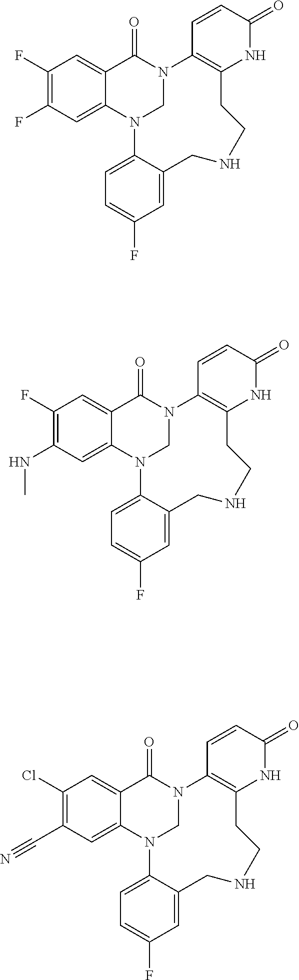 [SMILES: O=C1c2cc(F)c(F)cc2N2CN1c1ccc(=O)[nH]c1CCNCc1cc(F)ccc12 ; CNc1cc2c(cc1F)C(=O)N1CN2c2ccc(F)cc2CNCCc2[nH]c(=O)ccc21 ; N#Cc1cc2c(cc1Cl)C(=O)N1CN2c2ccc(F)cc2CNCCc2[nH]c(=O)ccc21] 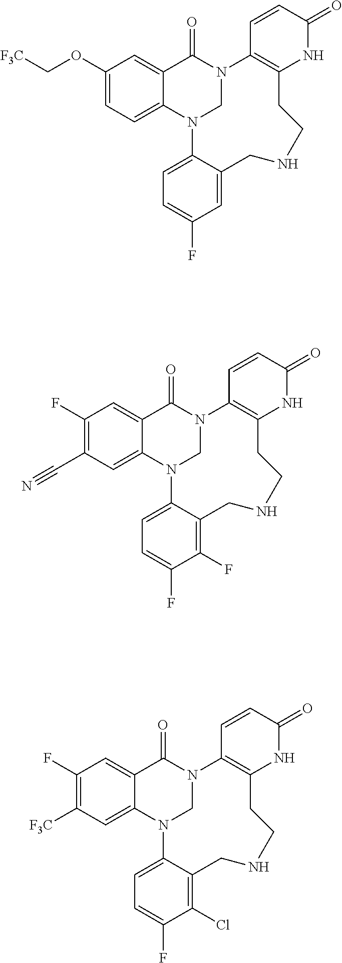 [SMILES: O=C1c2cc(OCC(F)(F)F)ccc2N2CN1c1ccc(=O)[nH]c1CCNCc1cc(F)ccc12 ; N#Cc1cc2c(cc1F)C(=O)N1CN2c2ccc(F)c(F)c2CNCCc2[nH]c(=O)ccc21 ; O=C1c2cc(F)c(C(F)(F)F)cc2N2CN1c1ccc(=O)[nH]c1CCNCc1c2ccc(F)c1Cl] 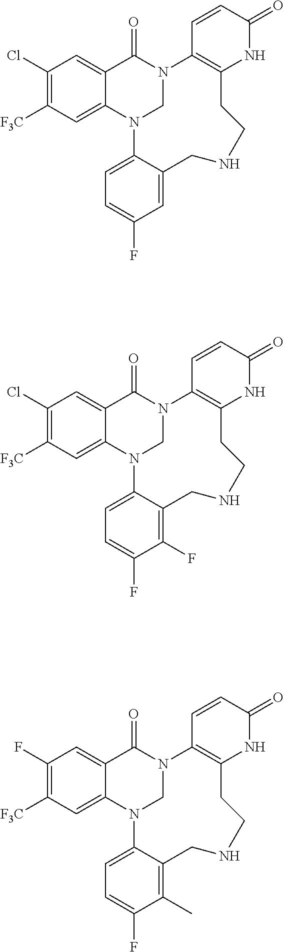 [SMILES: O=C1c2cc(Cl)c(C(F)(F)F)cc2N2CN1c1ccc(=O)[nH]c1CCNCc1cc(F)ccc12 ; O=C1c2cc(Cl)c(C(F)(F)F)cc2N2CN1c1ccc(=O)[nH]c1CCNCc1c2ccc(F)c1F ; Cc1c(F)ccc2c1CNCCc1[nH]c(=O)ccc1N1CN2c2cc(C(F)(F)F)c(F)cc2C1=O] 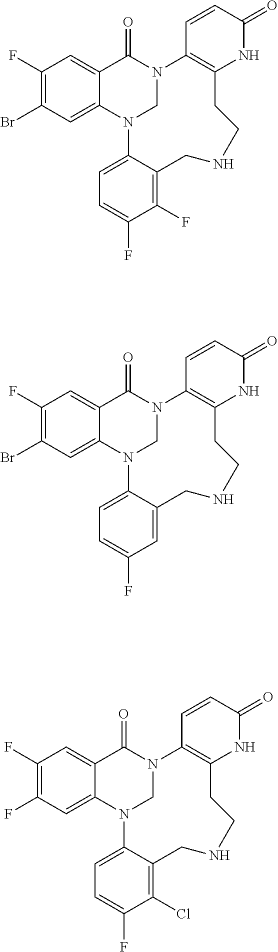 [SMILES: O=C1c2cc(F)c(Br)cc2N2CN1c1ccc(=O)[nH]c1CCNCc1c2ccc(F)c1F ; O=C1c2cc(F)c(Br)cc2N2CN1c1ccc(=O)[nH]c1CCNCc1cc(F)ccc12 ; O=C1c2cc(F)c(F)cc2N2CN1c1ccc(=O)[nH]c1CCNCc1c2ccc(F)c1Cl] 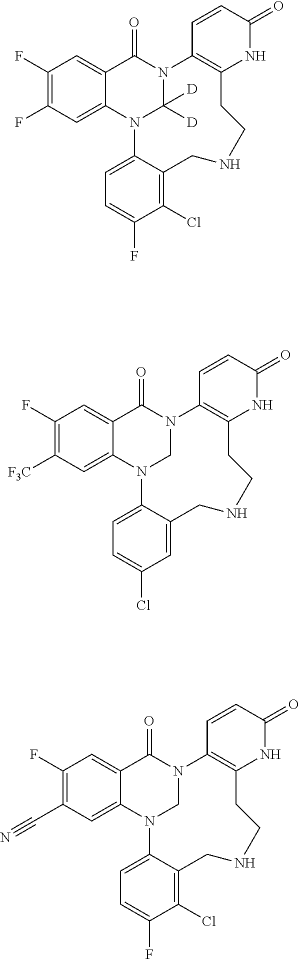 [SMILES: [2H]C1([2H])N2C(=O)c3cc(F)c(F)cc3N1c1ccc(F)c(Cl)c1CNCCc1[nH]c(=O)ccc12 ; O=C1c2cc(F)c(C(F)(F)F)cc2N2CN1c1ccc(=O)[nH]c1CCNCc1cc(Cl)ccc12 ; N#Cc1cc2c(cc1F)C(=O)N1CN2c2ccc(F)c(Cl)c2CNCCc2[nH]c(=O)ccc21] 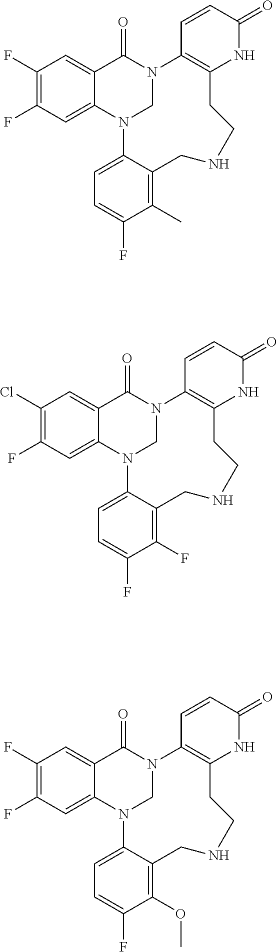 [SMILES: Cc1c(F)ccc2c1CNCCc1[nH]c(=O)ccc1N1CN2c2cc(F)c(F)cc2C1=O ; O=C1c2cc(Cl)c(F)cc2N2CN1c1ccc(=O)[nH]c1CCNCc1c2ccc(F)c1F ; COc1c(F)ccc2c1CNCCc1[nH]c(=O)ccc1N1CN2c2cc(F)c(F)cc2C1=O] 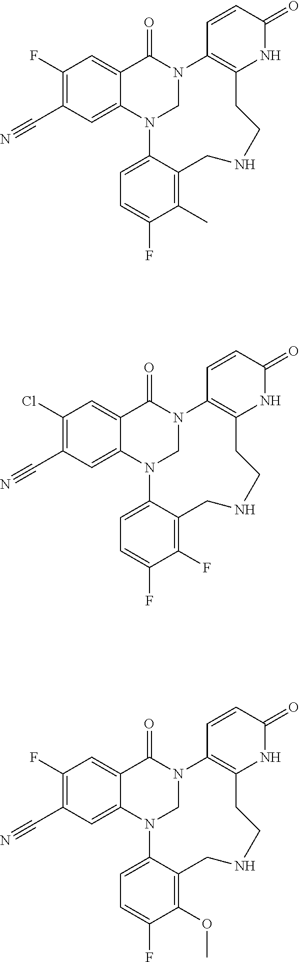 [SMILES: Cc1c(F)ccc2c1CNCCc1[nH]c(=O)ccc1N1CN2c2cc(C#N)c(F)cc2C1=O ; N#Cc1cc2c(cc1Cl)C(=O)N1CN2c2ccc(F)c(F)c2CNCCc2[nH]c(=O)ccc21 ; COc1c(F)ccc2c1CNCCc1[nH]c(=O)ccc1N1CN2c2cc(C#N)c(F)cc2C1=O] 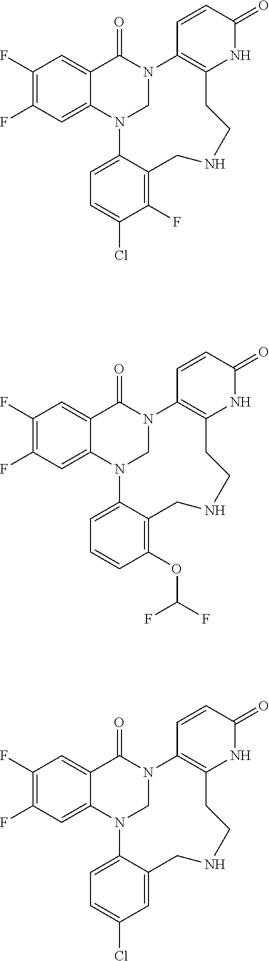 [SMILES: O=C1c2cc(F)c(F)cc2N2CN1c1ccc(=O)[nH]c1CCNCc1c2ccc(Cl)c1F ; O=C1c2cc(F)c(F)cc2N2CN1c1ccc(=O)[nH]c1CCNCc1c(OC(F)F)cccc12 ; O=C1c2cc(F)c(F)cc2N2CN1c1ccc(=O)[nH]c1CCNCc1cc(Cl)ccc12] 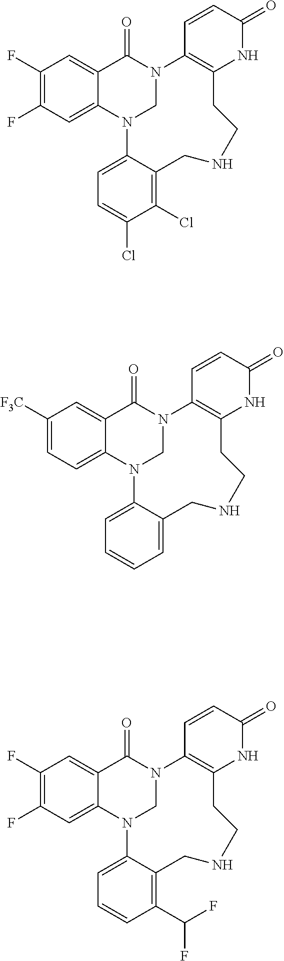 [SMILES: O=C1c2cc(F)c(F)cc2N2CN1c1ccc(=O)[nH]c1CCNCc1c2ccc(Cl)c1Cl ; O=C1c2cc(C(F)(F)F)ccc2N2CN1c1ccc(=O)[nH]c1CCNCc1ccccc12 ; O=C1c2cc(F)c(F)cc2N2CN1c1ccc(=O)[nH]c1CCNCc1c(C(F)F)cccc12] 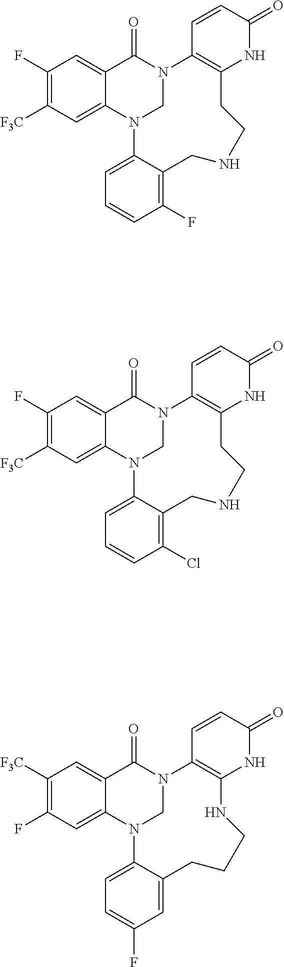 [SMILES: O=C1c2cc(F)c(C(F)(F)F)cc2N2CN1c1ccc(=O)[nH]c1CCNCc1c(F)cccc12 ; O=C1c2cc(F)c(C(F)(F)F)cc2N2CN1c1ccc(=O)[nH]c1CCNCc1c(Cl)cccc12 ; O=C1c2cc(C(F)(F)F)c(F)cc2N2CN1c1ccc(=O)[nH]c1NCCCc1cc(F)ccc12] 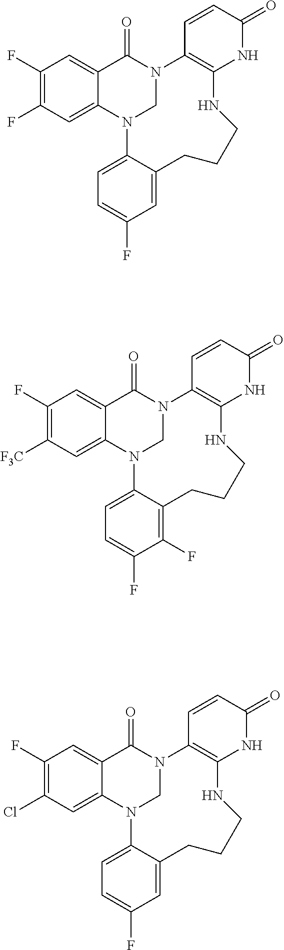 [SMILES: O=C1c2cc(F)c(F)cc2N2CN1c1ccc(=O)[nH]c1NCCCc1cc(F)ccc12 ; O=C1c2cc(F)c(C(F)(F)F)cc2N2CN1c1ccc(=O)[nH]c1NCCCc1c2ccc(F)c1F ; O=C1c2cc(F)c(Cl)cc2N2CN1c1ccc(=O)[nH]c1NCCCc1cc(F)ccc12] 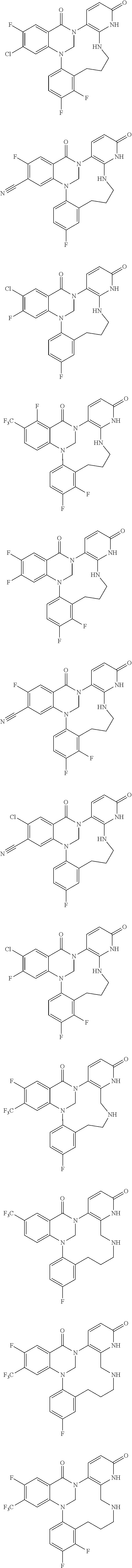 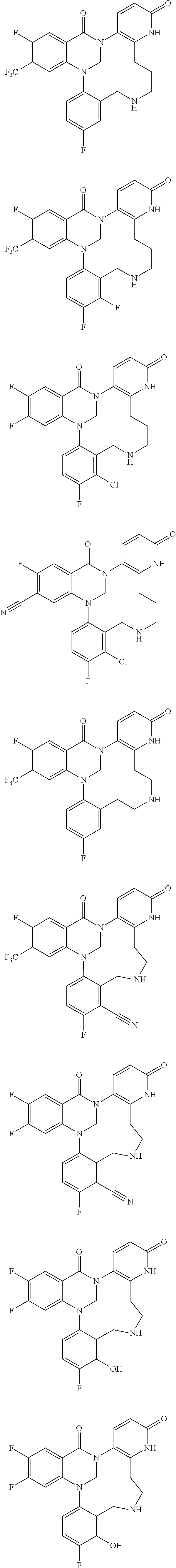 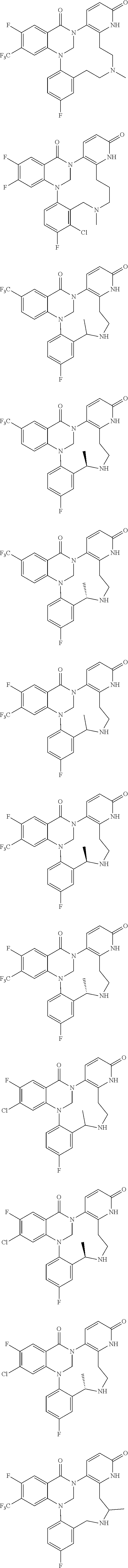 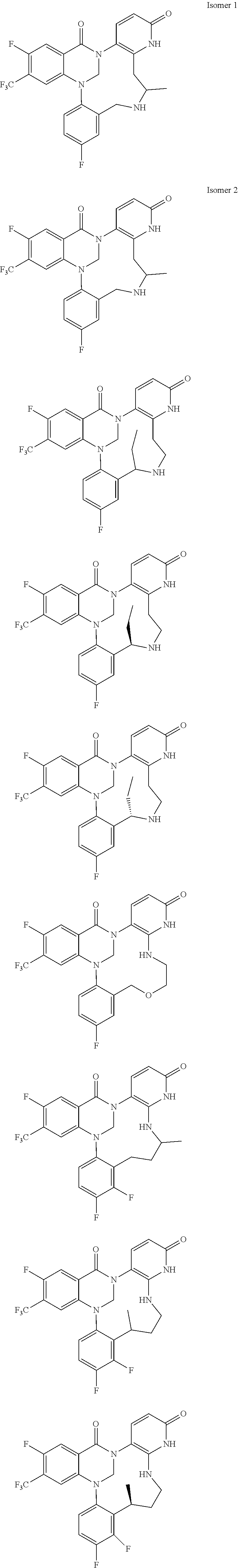 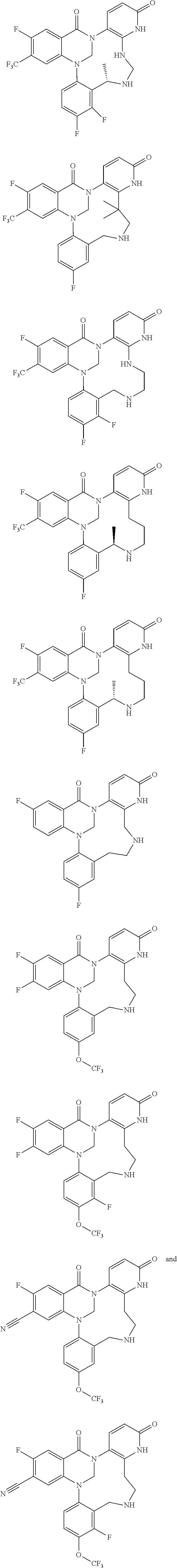
or a tautomer thereof, or a pharmaceutically acceptable salt thereof.

16. A compound selected from the group consisting of: 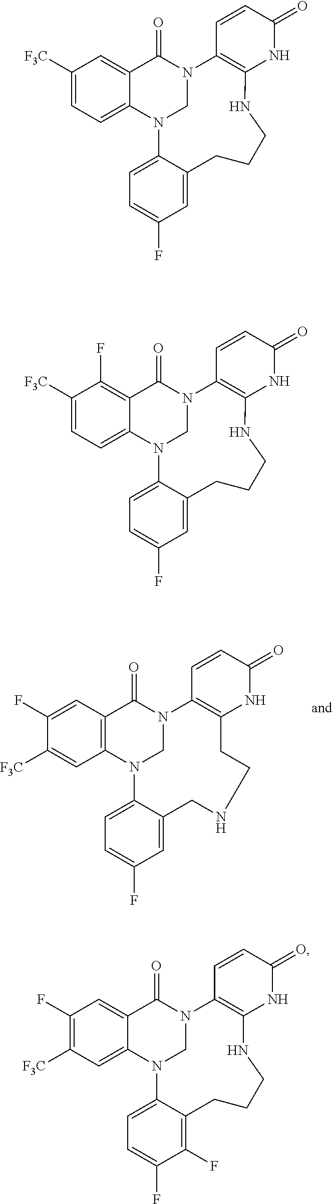 [SMILES: O=C1c2cc(C(F)(F)F)ccc2N2CN1c1ccc(=O)[nH]c1NCCCc1cc(F)ccc12 ; O=C1c2c(ccc(C(F)(F)F)c2F)N2CN1c1ccc(=O)[nH]c1NCCCc1cc(F)ccc12 ; O=C1c2cc(F)c(C(F)(F)F)cc2N2CN1c1ccc(=O)[nH]c1CCNCc1cc(F)ccc12 ; O=C1c2cc(F)c(C(F)(F)F)cc2N2CN1c1ccc(=O)[nH]c1NCCCc1c2ccc(F)c1F]
or a tautomer thereof, or a pharmaceutically acceptable salt thereof.

17. A pharmaceutical composition comprising the compound, or tautomer thereof or pharmaceutically acceptable salt thereof according to claim 1, and a pharmaceutically acceptable excipient.

18. A method of treatment of pain or a pain-associated disease in a human in need thereof, the method comprising administering to the human a compound according to claim 1, or a tautomer thereof, or a pharmaceutically acceptable salt thereof.

19. A method of treatment of atrial fibrillation in a human in need thereof, the method comprising administering to the human a compound according to claim 1, or a tautomer thereof, or a pharmaceutically acceptable salt thereof.

20-25. (canceled)

26. A method of treatment of a Naᵥ1.8-mediated disease, disorder, or condition in a human in need thereof, the method comprising administering a therapeutically effective amount of a compound according to claim 1, or a pharmaceutically acceptable salt thereof.

27. A method of treatment of a Naᵥ1.8-mediated disease, disorder, or condition in a human in need thereof, the method comprising administering to the human the pharmaceutical composition according to claim 17.

28. A method of treatment of pain or a pain-associated disease in a human in need thereof, the method comprising administering to the human the pharmaceutical composition according to claim 17.

29. A method of treatment of atrial fibrillation in a human in need thereof, the method comprising administering to the human-the pharmaceutical composition according to claim 17.
